(19) United States
(12) Patent Application Publication
(10) Pub. No.: US 2026/0125708 A1
(43) Pub. Date: May 7, 2026

(54) Engineered Integration Enzymes and Uses Thereof

(71) Applicant: Basecamp Research Ltd, London (GB)

(72) Inventor: Jesse Christine COCHRANE, Cambridge, MA (US)

(21) Appl. No.: 19/200,601

(22) Filed: May 6, 2025

Related U.S. Application Data
(60) Provisional application No. 63/643,230, filed on May 6, 2024.

(51) Int. Cl. C12N 15/90; C12N 5/074; C12N 5/0789; C12N 9/12; C12N 9/22; C12N 15/11; C12N 15/62; C12N 15/85

(52) U.S. Cl. CPC C12N 15/90; C12N 5/0647; C12N 5/0696; C12N 9/1276; C12N 9/222; C12N 15/111; C12N 15/62; C12N 15/85; C07K 2319/81; C12N 2310/20; C12N 2510/00; C12N 2800/22; C12Y 207/07049

(57) ABSTRACT

The present disclosure provides compositions comprising engineered integration enzymes/eLSR and methods of using the same. In certain embodiments, the engineered integration enzyme comprises mutation(s) that substantially maintain or enhance integration activity at a pair of cognate integration recognition sites, and substantially decrease off-target integration activity at a pair of off-target integration recognition sites, when compared to a corresponding large serine integrase without said one or more substitutions (cLSR). The eLSR may further comprise a stabilization domain that increases the stability of the integration enzyme as compared to integration enzymes not comprising the stabilization domain.

### 1. CROSS-REFERENCE TO RELATED APPLICATIONS

This application claims the benefit of U.S. Provisional Application No. 63/643,230, filed May 6, 2024, which is hereby incorporated in its entirety by reference herein.

### 2. SEQUENCE LISTING

The instant application contains a Sequence Listing which has been submitted by Patent Center and is hereby incorporated by reference in its entirety. Said XML copy, created on Sep. 11, 2025, is named 62809US_CRF_Sequence Listing.xml and is 1,196,506 bytes in size.

### 3. BACKGROUND

Programmable, efficient, and multiplexed genome integration of large, diverse DNA cargo independent of DNA repair remains an unsolved challenge of genome editing. Current gene integration approaches require double strand breaks that evoke DNA damage responses and rely on repair pathways that are inactive in terminally differentiated cells. Furthermore, CRISPR-based approaches that bypass double stranded breaks, such as Prime editing, are limited to modification or insertion of short sequences.

While targeted integration of large donor DNA sequences at specific target locations/genomic sites have been achieved using certain large serine integrases (LSRs), occasional integration of the donor DNA at unintended non-target locations/genomic sites can be problematic, in that two classes of potential genotoxic events may occur due to such integrase-mediated off-target integration—DNA mutagenesis and DNA structural variants formation. Specifically, DNA mutagenesis could potentially arise via DNA free-end formation from LSR cleavage or abortive integration. Meanwhile, DNA structural variants could potentially arise via cryptic recombination between cryptic integrase sites in the human genome leading to off-target cargo insertion, structural variants, or chromosomal rearrangements.

There is a need in the art for techniques which address and overcome these shortcomings and enable the co-delivery of gene editor constructs and associated donor templates for the insertion and/or deletion of large sequences into cells for therapeutic and circuit-based uses for broad purposes, across eukaryotic as well as prokaryotic systems.

### 4. SUMMARY

The present disclosure describes integration enzymes (e.g., engineered large serine integrase or eLSR) engineered such that upon being introduced into a cell, the integration enzyme has increased fidelity/specificity towards the cognate integration recognition sequences/sites at the target integration sequence, over off-target integration recognition sequences/sites at off-target integration sequences. The engineered large serine integrase (eLSR) described herein comprises one or more substitutions (e.g., substitutions in a zinc ribbon domain (ZD)) that substantially maintain or enhance integration activity at a pair of cognate integration recognition sites, and substantially decrease off-target integration activity at a pair of off-target integration recognition sites, when compared to a corresponding large serine integrase without said one or more substitutions (cLSR).

In certain embodiments, the one or more substitutions are in a zinc ribbon domain (ZD) of the cLSR.

In certain embodiments, the cLSR comprises an amino acid sequence that is at least 80% identical to any one of (a) SEQ ID NOs: 378-393; (b) SEQ ID NOs: 85-158 of WO2023/177424 (incorporated herein by reference); and (c) SEQ ID NOs: 1-16 and 163-1162 and 3166-3175 of WO2023/070031 (incorporated herein by reference).

In certain embodiments, the cLSR is a BxB1 polypeptide, SsuINT, SssINT, SscINT, Ssc2INT, SsdINT, SmcINT, UhmINT, SacINT, RsaINT, Rsa2INT, Bxb1, Tp91NT, Bt1INT, BceINT, BcyINT, SluINT, or a functional fragment/variant thereof.

In certain embodiments, the cLSR is a BxB1 polypeptide or a functional fragment/variant thereof.

In certain embodiments, the cLSR comprises an amino acid sequence having at least 80% sequence identity to SEQ ID NO: 388. In certain embodiments, the cLSR has an amino acid sequence of SEQ ID NO: 388.

In certain embodiments, the pair of cognate integration recognition sites are: an attB sequence and an attP sequence; or a modified AttB sequence and a modified AttP sequence.

In certain embodiments, the pair of off-target integration recognition sites are CAS031 attB sequence and CAS031 attP sequence; or CAS421 attB sequence and CAS421 attP sequence. See FIG. 59.

In certain embodiments, at least one of the pair of cognate integration recognition sites is integrated into a mammalian cell genome at a target DNA sequence. In certain embodiments, the at least one of the pair of cognate integration recognition sites is integrated into the mammalian cell genome at the target DNA sequence by: (1) programmable addition through site-specific targeting elements (PASTE) using a n attachment site-containing guide RNA (atgRNA) and a gene editor polypeptide; (2) homology directed repair (HDR), such as short-fragment homologous recombination (SFHR); (3) ligation-assisted homologous recombination (LAHR); (4) ligation-assisted Replacer (e.g., Replacer 1 or Replacer 2) editing; or, (5) non-homologous end joining (NHEJ), such as homology-independent targeted insertion (HITI).

In certain embodiments, the one or more substitutions are at a position corresponding to residue 315, residue 318, residue 319, and/or residue 320 of SEQ ID NO: 388.

In certain embodiments, the one or more substitutions do not include A315F, A315G, A315H, A3151, A315M, A315N, A315S, A315T, A315W, A315Y, G3181, G318K, G318R, and G318W.

In certain embodiments, the one or more substitutions are at a position corresponding to residue 315 of SEQ ID NO: 388, residue 318 of SEQ ID NO: 388, residue 319 of SEQ ID NO: 388, or residue 320 of SEQ ID NO: 388. In certain embodiments, the one or more substitutions are at a position corresponding to residue 315 of SEQ ID NO: 388. In certain embodiments, the one or more substitutions are at a position corresponding to residue 318 of SEQ ID NO: 388. In certain embodiments, the one or more substitutions are at a position corresponding to residue 319 of SEQ ID NO: 388. In certain embodiments, the one or more substitutions are at a position corresponding to residue 320 of SEQ ID NO: 388.

In certain embodiments, the one or more substitutions comprise or consist of a substitution to D (Asp), E (Glu) or R (Arg) at the position corresponding to residue 315 of SEQ ID NO: 388. In certain embodiments, the one or more substitutions comprise or consist of a substitution to R (Arg) at the position corresponding to residue 315 of SEQ ID NO: 388.

In certain embodiments, the one or more substitutions comprise or consist of a substitution to E (Glu) at the position corresponding to residue 318 of SEQ ID NO: 388.

In certain embodiments, the one or more substitutions comprise or consist of a substitution to Q (Gln) at the position corresponding to residue 319 of SEQ ID NO: 388.

In certain embodiments, the one or more substitutions comprise or consist of a substitution to E (Glu), Q (Gln), or R (Arg) at the position corresponding to residue 320 of SEQ ID NO: 388. In certain embodiments, the one or more substitutions comprise or consist of a substitution to E (Glu) at the position corresponding to residue 320 of SEQ ID NO: 388. In certain embodiments, the one or more substitutions comprise or consist of a substitution to R (Arg) at the position corresponding to residue 320 of SEQ ID NO: 388.

In certain embodiments, the one or more substitutions comprise or consist of two of the three positions corresponding to residue 315 of SEQ ID NO: 388, residue 319 of SEQ ID NO: 388, and residue 320 of SEQ ID NO: 388.

In certain embodiments, the one or more substitutions comprise or consist of the position corresponding to residue 315 of SEQ ID NO: 388, and the position corresponding to residue 320 of SEQ ID NO: 388.

In certain embodiments, the one or more substitutions comprise or consist of a substitution to D (Asp), E (Glu) or R (Arg) at the position corresponding to residue 315 of SEQ ID NO: 388; and a substitution to E (Glu), Q (Gln), or R (Arg) at the position corresponding to residue 320 of SEQ ID NO: 388.

In certain embodiments, the one or more substitutions comprise or consist of a substitution to D at the position corresponding to residue 315 of SEQ ID NO: 388; and a substitution to E at the position corresponding to residue 320 of SEQ ID NO: 388.

In certain embodiments, the one or more substitutions comprise or consist of a substitution to E at the position corresponding to residue 315 of SEQ ID NO: 388; and a substitution to E at the position corresponding to residue 320 of SEQ ID NO: 388.

In certain embodiments, the one or more substitutions comprise or consist of a substitution to R at the position corresponding to residue 315 of SEQ ID NO: 388; and a substitution to E at the position corresponding to residue 320 of SEQ ID NO: 388.

In certain embodiments, the one or more substitutions comprise or consist of a substitution to D at the position corresponding to residue 315 of SEQ ID NO: 388; and a substitution to R at the position corresponding to residue 320 of SEQ ID NO: 388.

In certain embodiments, the one or more substitutions comprise or consist of a substitution to E at the position corresponding to residue 315 of SEQ ID NO: 388; and a substitution to R at the position corresponding to residue 320 of SEQ ID NO: 388.

In certain embodiments, the one or more substitutions comprise or consist of a substitution to R at the position corresponding to residue 315 of SEQ ID NO: 388; and a substitution to R at the position corresponding to residue 320 of SEQ ID NO: 388.

In certain embodiments, the integration activity at the pair of cognate integration recognition sites is at least about 200-fold, 300-fold, 400-fold, 500-fold, 600-fold, 700-fold, 800-fold, 900-fold, 1000-fold or more compared to the off-target integration activity at the pair of off-target integration recognition sites.

In certain embodiments, the eLSR further has increased stability (e.g., half-life) compared to a control integration enzyme not engineered to have increased stability. The increase in stability extends the capacity of the integration enzyme to mediate integration.

In one embodiment, the present disclosure provides integration enzymes having enhanced fidelity towards cognate integration recognition sequences over non-cognate integration recognition sequences, and further engineered to have increased stability, for use in site-specific genetic engineering using Programmable Addition via Site-Specific Targeting Elements (PASTE) (see Ionnidi et al.; doi: 10.1101/2021.11.01.466786; the entirety of Ionnidi et al. is incorporated by reference), transposon-mediated gene editing, or other suitable gene editing or gene incorporation technology. Non-limiting examples of PASTE are also described in U.S. Pat. No. 11,572,556 and PCT Publication Nos. WO 2022/087235A and WO 2023/077148A1, each of which are hereby incorporated by reference in their entireties.

In typical embodiments, the engineered integration enzyme includes an integration enzyme or fragment thereof having mutation(s) that substantially maintain or enhance integration activity at a pair of cognate integration recognition sites, and substantially decrease off-target integration activity at a pair of off-target integration recognition sites, optionally an at least first stabilization domain, and further optionally a nuclear localization signal.

In certain embodiments, the eLSR further comprises (a) a first stabilization domain; and, (b) a first nuclear localization signal (NLS).

In some embodiments, the integration enzyme/eLSR or fragment thereof comprises integration enzyme, recombinase, or transposase activity.

In some embodiments, the integration enzyme is a large serine integrase.

In some embodiments, the engineered integration enzyme/eLSR binds to a cognate pair of integration recognition sites.

In some embodiments, the cognate pair of integration recognition sites is selected from: an attB and an attP and a modified AttB and a modified AttP.

In some embodiments, the engineered integration enzyme/eLSR further comprises a second stabilization domain.

In some embodiments, the first stabilization domain and/or second stabilization domain are positioned in the engineered integration enzyme such that the engineered integration enzyme and the first and/or second stabilization domains are an in-frame fusion.

In some embodiments, the first stabilization domain and/or the second stabilization domain are located N-terminal to the eLSR, C-terminal to the eLSR, and/or between two consecutive amino acid residues of the eLSR.

In some embodiments, the first stabilization domain and/or second stabilization domain are located N-terminus to the integration enzyme/eLSR or fragment thereof.

In some embodiments, the first stabilization domain and/or second stabilization domain are located C-terminus to the integration enzyme/eLSR or fragment thereof.

In some embodiments, the first stabilization domain and/or second stabilization domain are located between two consecutive amino acid residues in the amino acid sequence of the integration enzyme/eLSR or fragment thereof.

In some embodiments, the two consecutive amino acid residues in the amino acid sequence of the integration enzyme/eLSR are located in a N-terminal catalytic domain; a recombinase domain, or one or more zinc ribbon domain.

In some embodiments, the two consecutive amino acid residues in the amino acid sequence of the integration enzyme correspond amino acid residues between the N-terminal catalytic domain and the recombinase domain or between the recombinase domain and one of the zinc ribbon domains.

In certain embodiments, the two consecutive amino acid residues of the eLSR are located in a N-terminal catalytic domain (NTD) of the eLSR; in a recombinase domain (RD) of the eLSR; in a zinc ribbon domain (ZD) of the eLSR; between the NTD and the RD of the eLSR; or between the RD and the ZD of the eLSR

In some embodiments, the stabilization domain is selected from: a stabilon motif and a exin21 motif. In certain embodiments, the stabilization domain comprises a stabilon motif and/or an exin2l motif.

In some embodiments, the stabilization domain comprises the stabilon motif, and wherein the stabilon motif has at least 80% sequence identity to a sequence of

(SEQ ID NO: 583
KDKDKKSDGKDSQKK
or
(SEQ ID NO: 584)
KDKKSDGKDSQKK.

In some embodiments, the stabilization domain comprises the exin2l motif, and wherein the exin2l motif has a sequence of QPRFAAA (SEQ ID NO: 585) or a sequence of QPRFAAA (SEQ ID NO: 585) within one, two or three amino acid substitutions.

In some embodiments, the second stabilization domain comprises a stabilon motif and/or an exin21 motif.

In some embodiments, the engineered integration enzyme further comprises one or more additional stabilization domains.

In some embodiments, the one or more additional stabilization domains comprises a stabilon motif and/or a exin2l motif.

In some embodiments, the engineered integration enzyme further comprises at least a first tag.

In some embodiments, the engineered integration enzyme further comprises a second tag.

In some embodiments, the first tag and/or the second tag are position in the engineered integration enzyme/eLSR such that the engineered integration enzyme/eLSR and the first and/or second tag are an in-frame fusion.

In some embodiments, the first tag and/or second tag are selected from: an HA tag, a HiBit tag, strep tag, and SUMO tag.

In some embodiments, the engineered integration enzyme/eLSR further comprises one or more linkers.

In some embodiments, the one or more linkers is a glycine serine linker selected from:

(SEQ ID NO: 677)
GS, GSG, or GGGS.

In some embodiments, the engineered integration enzyme/eLSR comprises an orientation from N-terminus to C-terminus: N-I-S; I-S-N; S-I-N; N-S-I; N-X₁-I-S; I-X₁-S-N; S-X₁-I-N; N-X₁-S-I; N-I-X₁-S; I-S-X₁-N; S-I-X₁-N; N-S-X₁-I; N-X₁-I-X₂-S; I-X₁-S-X₂-N; S-X₁-I-X₂-N; or N-X₁-S-X₂-I; wherein I is the eLSR without the linker, the stabilization domain, and the first NLS; X₁ is a first linker (when present); S is the stabilization domain; X₂ is a second linker (when present); and N is the first NLS (when present).

In certain embodiments, the eLSR further comprises: (a) a first amino acid modification that increases the stability of the eLSR as compared to the eLSR without said first amino acid modification; and; and (b) a second nuclear localization signal (NLS).

In some embodiments, the first amino acid modification is located in a degron motif.

In some embodiments, the first amino acid modification is an amino acid substitution, an insertion, a deletion, or a combination thereof.

In some embodiments, the first amino acid modification is an amino acid substitution of L275V in SEQ ID NO: 388.

In some embodiments, the engineered integration enzyme/eLSR is linked to a gene editor polypeptide.

In some embodiments, the C-terminus of the engineered integration enzyme/eLSR is linked to the gene editor polypeptide.

In some embodiments, the C-terminus of the gene editor polypeptide is linked to the engineered integration enzymes/eLSR.

In some embodiments, the engineered integration enzyme/eLSR is linked to the gene editor polypeptide by in-frame fusion.

In some embodiments, the engineered integration enzyme/eLSR is linked to the gene editor polypeptide by a linker.

In some embodiments, the linker is a peptide fused in-frame between the engineered integration enzyme/eLSR and the gene editor polypeptide.

In some embodiments, the one or more linkers is selected from: Table 3.

In some embodiments, the gene editor polypeptide comprises a DNA binding domain and a reverse transcriptase.

In another aspect, this disclosure features a polynucleotide comprising a nucleic acid sequence encoding any of the engineered integration enzymes/eLSR described herein or any of the engineered integration enzymes/eLSRs linked to the gene editor polypeptides described herein.

In some embodiments, the nucleic acid sequence encoding the eLSR and/or the fusion is codon optimized (e.g., codon-optimized for expression in a mammalian cell, such as a human cell).

In some embodiments, the codon optimization is performed using an algorithm.

In some embodiments, the nucleic acid sequence encoding the eLSR and/or the fusion is optimized based on secondary structure that confers increased stability of the polynucleotide.

In some embodiments, the nucleic acid sequence of the engineered integration enzyme/eLSR is optimized based on a linear design algorithm.

In another aspect, this disclosure features a vector comprising any of the nucleic acid sequences described herein.

In another aspect, this disclosure features a host cell comprising any of the the vectors described herein.

In another aspect, this disclosure features a fusion protein, comprising: (a) a DNA binding domain, optionally comprising a nickase activity; (b) a reverse transcriptase; and (c) any of the engineered integration enzymes/eLSR described herein, wherein at least any two of elements (a), (b), or (c) are linked via at least a first C-terminal linker.

In some embodiments, the C-terminal linker comprises a sequence in Table 3.

In another aspect, this disclosure features a polynucleotide comprising any of the nucleic acid sequences encoding the all-in-one fusion protein.

In another aspect, this disclosure features a vector comprising any of the nucleic acid sequences described herein.

In another aspect, this disclosure features a host cell comprising any of the vectors described herein.

In another aspect, this disclosure features a system for site-specifically integrating a donor polynucleotide template into a mammalian cell genome at a target DNA sequence, comprising: an attachment site containing gRNA (atgRNA) comprising at least a portion of an at least first integration recognition site; a gene editor polypeptide comprising a DNA binding nickase domain linked to a reverse transcriptase domain capable of incorporating the integration recognition site into the target DNA sequence, any of the engineered integration enzymes/eLSR described herein; and a donor polynucleotide template linked to a sequence that is an integration cognate of the integration recognition site present in the atgRNA, whereby the gene editor polypeptide site-specifically integrates the integration recognition site into the target DNA sequence, whereby the engineered integration enzyme/eLSR integrates the donor polynucleotide template into the target DNA sequence at the integration recognition site.

In some embodiments, the first atgRNA comprises: (i) a domain that is capable of guiding the gene editor polypeptide to the target DNA sequence; and (ii) a reverse transcriptase (RT) template that comprises at least a portion of an at least first integration recognition site, whereby the at least portion of the at least first integration recognition site is integrated into the genome of the cell at the target sequence.

In some embodiments, the system further comprises a second atgRNA.

In some embodiments, the first atgRNA and the second atgRNA are an at least first pair of atgRNAs, wherein the at least first pair of atgRNAs have domains that are capable of guiding the gene editor polypeptide to the target DNA sequence; the first atgRNA further includes a first RT template that comprises at least a portion of an at least first integration recognition site; the second atgRNA further includes a second RT template that comprises at least a portion of the first integration recognition site, and the first atgRNA and the second atgRNAs collectively encode the entirety of the first integration recognition site, whereby the least first integration recognition site is integrated into the genome of the cell at the target sequence.

In some embodiments, where, upon introducing the system into the cell, the engineered integration enzyme/eLSR enhances integration fidelity as well as efficiency of the donor polynucleotide template at the site-specifically integrated integration recognition site as compared to the integration efficiency of a system using a non-engineered integration enzyme (a corresponding cLSR) to integrate donor polynucleotide template at the site-specifically integrated integration recognition site.

In another aspect, this disclosure features a method for site-specifically integrating a donor polynucleotide template into a mammalian cell genome at a target DNA sequence, comprising: incorporating an integration recognition site into the genome by delivering into the cell: an attachment site containing guide RNA (atgRNA) comprising at least a portion of an at least first integration recognition site; and a gene editor polypeptide or polynucleotide encoding a gene editor polypeptide, wherein the gene editor polypeptide comprises a DNA binding nickase domain linked to a reverse transcriptase domain and is capable of incorporating the integration recognition site into the target DNA sequence; and optionally, a nicking gRNA; and integrating the donor polynucleotide template into the genome by delivering into the cell: any of the engineered integration enzymes/eLSR described herein; and a donor polynucleotide template, wherein the donor polynucleotide template is linked to a sequence that is an integration cognate of the integration recognition site present in the atgRNA, and wherein the donor polynucleotide template is integrated into the genome at the incorporated genomic integration recognition site by the integration enzyme.

In some embodiments, the atgRNA, the gene editor polypeptide or polynucleotide encoding the gene editor polypeptide, the optional nicking gRNA, the engineered integration enzyme/eLSR, and the donor polynucleotide template are introduced into the cell concurrently.

In some embodiments, the first atgRNA comprises: (i) a domain that is capable of guiding the gene editor polypeptide to the target DNAsequence; and (ii) a reverse transcriptase (RT) template that comprises at least a portion of an at least first integration recognition site, whereby the at least portion of the at least first integration recognition site is integrated into the genome of the cell at the target sequence.

In some embodiments, the method further comprises a second atgRNA.

In some embodiments, the first atgRNA and the second atgRNA are an at least first pair of atgRNAs, wherein the at least first pair of atgRNAs have domains that are capable of guiding the gene editor polypeptide to the target DNA sequence; the first atgRNA further includes a first RT template that comprises at least a portion of an at least first integration recognition site; the second atgRNA further includes a second RT template that comprises at least a portion of the first integration recognition site, and the first atgRNA and the second atgRNAs collectively encode the entirety of the first integration recognition site, whereby the at least first integration recognition site is integrated into the genome of the cell at the target sequence.

In some embodiments, the method enhances integration fidelity and efficiency of the donor polynucleotide template at the site-specifically integrated integration recognition stie at the double-stranded target DNA sequence as compared to the integration efficiency of the donor polynucleotide template at the site-specifically integrated recognition site when using a method that does not comprise the engineered integration enzyme/eLSR.

In some embodiments, the cell is in a human subject. In some embodiments, the human subject has a disease or condition selected from the group consisting of phenylketonuria (PKU), atherosclerotic cardiovascular disease (ASCVD), homocystinuria, and hemochromatosis.

In some aspects, disclosed herein is a use of the fusion protein, the polynucleotide, the vector, and/or the system of any preceding aspect in treatment of a disease or condition in a subject, wherein the disease or condition is selected from the group consisting of phenylketonuria (PKU), atherosclerotic cardiovascular disease (ASCVD), homocystinuria, and hemochromatosis.

In some aspects, disclosed herein is a cell comprising the fusion protein, the polynucleotide, the vector, and/or the system of any preceding aspect. In some embodiments, the cell is a pleiopluripotent cell (such as an induced pluripotent stem cell) or a hematopoietic cell differentiated from a pleiopluripotent cell. In some embodiments, the hematopoietic cell is a natural killer cell (NK) (such as an iNK cell).

In some aspects, disclosed herein is a use of the cell of any preceding aspect (e.g., iPSC or iNK cell) in treatment of a disease or condition in a subject, wherein the disease or condition is Systemic Lupus Erythematosus (SLE), Lupus Nephritis (LN), IgA Nephropathy (IgAN), Membranous nephropathy (MN), Minimal change disease (MCD), ANCA Vasculitis, Myasthenia Gravis (MG), Systemic sclerosis (SS), C3 glomerulopathy (C3GN), Idiopathic thrombocytopenic purpura (ITP), Multiple Sclerosis (MS), Guillain-Barre syndrome (GBS), Chronic Inflammatory Demyelinating polyradiculoneuropathy (CIDP), Pemphigus Vulgaris (PV), Bullous Pemphigoid (BP), Autoimmune Hepatitis (AIH), Autoimmune Hemolytic Anemia (AIHA), Dermatomyositis (DM), Polymyositis (PM), Scleroderma, Neuromyelitis optica spectrum disorders (NMOSD), Myelin oligodendrocyte glycoprotein antibody associated disease (MOGAD), Churg-Strauss syndrome/eosinophilic granulomatosis with polyangiitis (EGPA), CREST syndrome/limited cutaneous systemic sclerosis, Devic's disease/neuromyelitis optica (NMO), Granulomatosis with polyangiitis (GPA)/Wegener's granulomatosis, Henoch-Schonlein purpura/IgA vasculitis, autoimmune thrombocytopenic purpura/autoimmune thrombocytopenia, or Transverse Myelitis™. In some embodiments, the disease or condition is selected from the group consisting of systemic lupus erythematosus (SLE), IgA nephropathy, membranous nephropathy, and minimal change disease.

### 5. BRIEF DESCRIPTION OF THE DRAWINGS

These and other features, aspects, and advantages of the present invention will become better understood with regard to the following description, and accompanying drawings, where:

FIGS. 1-53 provide embodiments concerning the rate of cargo integration by integrase (such as wild-type integrase comprising Large Serine Integrases (LSRs) and their engineered high-fidelity variants thereof) into target DNA/genomic locations, while FIGS. 54-xxx provide embodiments concerning the fidelity of cargo integration by integrase (such as the engineered LSRs, or eLSRs) into the intended target DNA/genomic locations, as opposed to unintended/undesired integration into non-target DNA/genomic locations. It should be understood that any one embodiment, such as embodiments described solely under one aspect of the invention, can be combined with any other one or more additional embodiments, unless such combination is expressly disclaimed or improper.

FIG. 1 shows a non-limiting illustration of a gene editor construct packaged within a lipid nanoparticle (LNP).

FIG. 2 illustrates the donor template (i.e., “cargo” or “payload” or “template polynucleotide”)) packaged within a vector.

FIG. 3 illustrates integrase-mediated self-circularization of the donor template (template polynucleotide) within viral genome. The circularized donor template is capable of being genomically incorporated into an orthogonal integrase target recognition site (i.e., “beacon”).

FIG. 4 shows non-limiting illustrations of a gene editor construct packaged within a lipid nanoparticle and an atgRNA, ngRNA, and donor template (i.e., template polynucleotide encoding a gene of interest) packaged within a vector. GOI=gene of interest. PGI=programmable gene insertion. U6=U6 promoter. atgRNA=attachment site-containing guide RNA (atgRNA).

FIG. 5 shows non-limiting illustrations of a gene editor construct (e.g., mRNA encoding PE2-BxB1) and a nicking guide RNA (ngRNA) packaged within a lipid nanoparticle (LNP) and an atgRNA and donor template (i.e., template polynucleotide encoding a gene of interest) packaged within a vector.

FIGS. 6A-6B show non-limiting illustrations of three self-complementary AAV (scAAV) genomes capable of recombinase/integrase-mediated self-circularization. FIG. 6A shows the structure of the three self-complementary AAV (scAAV) genomes capable of recombinase/integrase-mediated self-circularization. FIG. 6B shows non-limiting examples of sequences that enable self-circularization (e.g., LoxP AttP GT (SEQ ID NO: 568 and SEQ ID NO: 569); FRT AttP GT (SEQ ID NO: 570 and SEQ ID NO: 571); and AttB CC AttP GT (SEQ ID NO: 572 and SEQ ID NO: 573)). GT indicates an AttP site with a GT dinucleotide. AttB CC indicates an AttB site with a CC dinucleotide. LoxP=a LoxP recombinase recognition site. FRT=a FRT recombinase recognition site.

FIG. 7 shows a non-limiting illustration of recombinase/integrase-mediated intramolecular circularization products.

FIGS. 8A-8B show non-limiting illustrations of a ddPCR assay and intramolecular circularization ddPCR detection probes. FIG. 8A shows a non-limiting illustration of the ddPCR strategy. FIG. 8B shows non-limiting examples of the universal probe (SEQ ID NO: 574 and SEQ ID NO: 575) and an AttR probe (SEQ ID NO: 576 and SEQ ID NO: 577) that can be used in the assay shown in FIG. 8A.

FIG. 9 shows a non-limiting illustration of a pDNA genome and AAV transfection and screening protocol.

FIG. 10 shows data for circularization of AAV pDNA and packaged AAV genomic DNA with Bxb1.

FIG. 11 shows data for Cre-, FLPe-, and Bxb1-mediated circularization of AAV pDNA confirmed by ddPCR.

FIG. 12 shows Cre-, FLPe-, and Bxb1-mediated circularization of packaged AAV confirmed by ddPCR

FIG. 13 shows percent circularization between the Bxb1-mediated attR scar ddPCR probe (“attR probe” described in FIG. 8B) and the universal ddPCR probe (“universal probe” described in FIG. 8B).

FIGS. 14A-14E shows analysis of AttP variants. FIG. 14A shows a non-limiting schematic of AttP mutations tested for improving integration efficiency (SEQ ID NOS: 394 and 540-542, respectively, in order of appearance). FIG. 14B shows integration efficiencies of wildtype and mutant AttP sites across a panel of AttB lengths. FIG. 14C shows a non-limiting schematic of multiplexed integration of different cargo sets at specific genomic loci. Three fluorescent cargos (GFP, mCherry, and YFP) are inserted orthogonally at three different loci (ACTB, LMNB1, NOLC1) for in-frame gene tagging. FIG. 14D shows orthogonality of top 4 AttB/AttP dinucleotide pairs evaluated for GFP integration with PASTE at the ACTB locus. FIG. 14E shows efficiency of multiplexed PASTE insertion of combinations of fluorophores at ACTB, LMNB1, and NOLC1 loci. Data are mean (n=3) s.e.m.

FIG. 15 illustrates a schematic of single atgRNA and dual atgRNA approaches for beacon placement (“integration recognition site”).

FIG. 16 shows percent beacon placement in primary mouse hepatocytes (PMH) following delivery of mRNA to deliver a polynucleotide encoding a gene editor polynucleotide construct and an AAV to deliver the first and second atgRNA according to the following conditions: (i) concurrent delivery (“co-dose”), (ii) AAV delivery followed by a “1-day delay” before delivery of the mRNA, or (iii) AAV delivery followed by a “2-day delay” before delivery of the mRNA.

FIG. 17 shows percent beacon placement in primary human hepatocytes (PHH) following delivering of mRNA to deliver a polynucleotide encoding a gene editor polynucleotide construct and an AAV to deliver the first and second atgRNA. The mRNA and AAV were delivered concurrently.

FIG. 18 shows percent in vivo beacon placement in the Nolc1 locus of mice following delivery of a polynucleotide encoding a gene editor polynucleotide construct using a lipid nanoparticle (LNP) and a first atgRNA and second atgRNA using an AAV. % BP=% beacon placement. LNP were administered at doses of 0.5 mg/kg, 1.5 mg/kg, 3 mg/kg, and 5 mg/kg. AAV was administered at 1E11, 3E11, or 1E12 viral genomes (vg) per animal. LNP #F1=LNP formulation #1. LNP #F2=LNP formulation #F2. LNP #F3=LNP formulation #F3.

FIG. 19 show percent in vivo integration of a template polynucleotide in AttP mice following delivering of the Bxb1 using adenovirus (AdV) and the template polynucleotide using an AAV (“AAV Cargo”). Bxb1 Adv was administered to the mice at a dose of either 3E10 or 1E11 vector genomes (vg) per animal. AAV Cargo was administered to the mice at a dose of 1E12.

FIG. 20A shows ddPCR data for percent in vivo beacon placement in the Nolc1 locus of neonatal mice at eight days post-delivery of a single dose of a mixture of two LNPs. First LNP contained mRNA encoding a prime editing system and a first synthetic atgRNA (atgRNA1) at a 1:1 ratio. Second LNP contained mRNA encoding a prime editing system and a second synthetic atgRNA (atgRNA2) at a 1:1 ratio. Each of the first and second atgRNAs targeted the mouse Nolc1 locus, encoded a portion of an integration recognition site (“beacon”), and together included a 6 bp overlap. The first and second LNPs were combined 1:1 as mixture and administered at either 1 mg/kg or 3 mg/kg. LNP #F2=LNP formulation #F2.

FIG. 20B show NGS data for percent in vivo beacon placement in the Nolc1 locus of the same neonatal mice and treatment conditions as described in FIG. 20A. NGS data shows beacon placement eight days after administration of the LNP mixture. LNP #F2=LNP formulation #F2.

FIG. 20C shows NGS data for percentage of in vivo beacons placed in the Nolc1 NGS data is for the same mice with the same treatment conditions as described in FIG. 20A. NGS data shows data for eight days after administration of the LNP mixture. LNP #F2=LNP formulation #F2.

FIG. 21A shows ddPCR data for percent in vivo beacon placement in the Nolc1 locus of neonatal mice at 6 weeks post-delivery of a single dose of a mixture of two LNPs. First LNP contained mRNA encoding a prime editing system and a first synthetic atgRNA (atgRNA1) at a 1:1 ratio. Second LNP contained mRNA encoding a prime editing system and a second synthetic atgRNA (atgRNA2) at a 1:1 ratio. Each of the first and second atgRNAs targeted the mouse Nolc1 locus, encoded a portion of an integration recognition site (“beacon”), and together included a 6 bp overlap. The first and second LNPs were combined 1:1 as mixture and administered at either 1 mg/kg or 3 mg/kg. LNP #F2=LNP formulation #F2.

FIG. 21B shows NGS data for percent in vivo beacon placement in the Nolc1 locus of the same neonatal mice and treatment conditions as described in FIG. 21A. NGS data shows beacon placement 6 weeks after administration of the LNP mixture. LNP #F2=LNP formulation #F2.

FIG. 21C shows NGS data for percentage of in vivo beacons placed in the Nolc1 locus that included the expected integration recognition site. Data is from the same mice with the same treatment conditions as described in FIG. 22A. NGS data shows data at 6 weeks after administration of the LNP mixture. LNP #F2=LNP formulation #F2.

FIG. 22A shows ddPCR data for percent in vivo beacon placement in the Factor IX (“mF9”) locus of 6-8 week old mice at day 8 post-delivery of a single dose of a mixture of two LNPs. First LNP contained mRNA encoding a prime editing system and a first synthetic atgRNA (atgRNA1) at a ratio of 1:0.5, 1:1, or 1:2. Second LNP contained mRNA encoding a prime editing system and a second synthetic atgRNA (atgRNA2) at a ratio of 1:1, 1:0.5, or 1:2. Each of the first and second atgRNAs targeted the mouse Factor IX locus, encoded a portion of an integration recognition site (“beacon”), and together included a 6 bp overlap. The first and second LNPs were combined 1:1 as mixture with the final ratio of mRNA:atgRNA1:atgRNA2 at 1:0.25:0.25; 1:0.5:0.5, or 1:1:1. LNP #F2=LNP formulation #F2.

FIG. 22B shows NGS data for percent in vivo beacon placement in the mF9 locus of the same neonatal mice and treatment conditions as described in FIG. 22A. NGS data shows beacon placement 8 days after administration of the LNP mixture. LNP #F2=LNP formulation #F2.

FIG. 22C shows NGS data for percent of in vivo beacons placed in the mF9 locus that included the expected integration recognition site. Data is from the same mice with the same treatment conditions as described in FIG. 22A. NGS data shows data at 8 days after administration of the LNP mixture. LNP #F2=LNP formulation #F2.

FIG. 23 shows schematics for non-limiting examples of engineered integrases. Abbreviations: 5′ UTR=5′ untranslated region; XBG=Xenopus beta globin; NLS=nuclear localization signal; BxB1=BxB1 integration enzyme; HA=HA Tag; HiBit=tag; 3′ UTR=3′ untranslated region; 80A's=poly A tail comprising 80 adenines (SEQ ID NO: 721). L275V indicates a L to V substitution at position 275 of BxB1 (SEQ ID NO: 388).

FIGS. 24A-24C show PGI data for the engineered integration enzymes described in FIG. 23. FIG. 24A shows ddPCR data for beacon placement. FIG. 24B shows ddPCR for integration %. FIG. 24C shows ddPCR data for beacon occupancy %.

FIG. 25 shows schematics for non-limiting examples of engineered integrases having a codon optimized coding sequence for BxB1 and a split polyA. 5′ UTR=5′ untranslated region; XBG=Xenopus beta globin; NLS=nuclear localization signal; BxB1=BxB1 integration enzyme; HA=HA Tag; HiBit=tag; 3′ UTR=3′ untranslated region; 30-70 pA's=polyA tail comprising 30-70 adenines (SEQ ID NO: 722). 80A's=poly A tail comprising 80 adenines (SEQ ID NO: 721).

FIG. 26 shows % Occupancy (an indicator of PGI) in cells treated with the indicated engineered BxB1 integration enzymes (e.g., as described in FIG. 25) at high dose (1.3 pmol) or low does (0.2 pmol).

FIGS. 27A-27D show % Beacon Occupancy (an indicator of PGI) in cells treated with mRNA encoding the indicated engineered BxB1 integration enzymes: PL760 (FIG. 27A), PL1303 (FIG. 27B), PL1304 (FIG. 27C), and PL1305 (FIG. 27D). mRNA was introduced at 1000 ng, 500 ng, 250 ng, and 0 ng (control).

FIG. 28 shows schematics for non-limiting examples of engineered integrases. 5′ UTR=5′ untranslated region; XBG=Xenopus beta globin; NLS=nuclear localization signal; BxB1=BxB1 integration enzyme; HA=HA Tag; HiBit=tag; 3′ UTR=3′ untranslated region; 80A's=poly A tail comprising 80 adenines (SEQ ID NO: 721); Strep=streptavidin tag; Stabilion=stabilion peptide; and Sumo=sumo peptide.

FIG. 29A shows PGI % in primary human hepatocytes (PHH) transfected with 250 ng of the indicated mRNAs: PL1303; PL1325: PL1326; PL1327; and PL1305 as well as controls Beacon only, nCas9-RT only and untreated cells.

FIG. 29B shows Beacon Occupancy % (an indicator of PGI) in primary human hepatocytes (PHH) transfected with 250 ng of the indicated mRNAs: PL1303; PL1325; PL1326; PL1327; and PL1305 as well as controls Beacon only, nCas9-RT only and untreated cells.

FIG. 29C shows PGI % in primary human hepatocytes (PHH) transfected with the indicated mRNAs: PL1303; PL1305; and PL1325 as well as controls Beacon only, nCas9-RT only and untreated cells.

FIG. 29D shows Beacon Occupancy % (an indicator of PGI) in primary human hepatocytes (PHH) transfected with the indicated mRNAs: PL1303; PL1305; and PL1325 as well as controls Beacon only, nCas9-RT only and untreated cells.

FIG. 30A shows a non-limiting workflow for using the engineered integration enzymes for programmable gene insertion, for example, in a pluripotent stem cell.

FIG. 30B shows schematics for non-limiting examples of engineered integrases. 5′ UTR=5′ untranslated region; XBG=Xenopus beta globin; NLS=nuclear localization signal; BxB1=BxB1 integration enzyme; HA=HA Tag; HiBit=tag; 3′ UTR=3′ untranslated region; 80A's=poly A tail comprising 80 adenines (SEQ ID NO: 721); Stabilion=stabilion peptide.

FIG. 30C shows cell viability data in iPSCs (clones 52 and 17) at day 3 after transduction for each of the conditions indicated on the x-axis.

FIG. 30D shows ddPCR data for percent (%) PGI in iPSC (clones 52 and 17) at day 3 after transduction for each of the conditions indicated on the x-axis.

FIG. 31A shows ddPCR data for percent (%) PGI at day 3 and day 6 for iPSC clone 52 for each of the conditions indicated on the x-axis.

FIG. 31B shows flow cytometry plots of side-scatter versus FITC-A for each of the indicated conditions. FITC-A is an indicator PGI.

FIG. 32A shows a schematic of non-limiting examples of engineered BxB1 integrases assessed for their ability to mediate PGI in hematopoietic stem cells (HSCs).

FIG. 32B shows ddPCR data for percent (%) PGI in HSCs for each of the conditions indicated on the x-axis (see engineered BxB1 integrases described in FIG. 32A).

FIG. 33 shows a schematic of non-limiting examples of engineered BxB1 integrases.

FIG. 34 shows ddPCR data for % Beacon Occupancy (an indicator of PGI) for each of the conditions indicated on the x-axis.

FIG. 35 shows a schematic of engineered BxB1 integrases selected for further study.

FIG. 36A shows a schematic of a BxB1 integrase with identification of the location of two predicted degron motifs as well as the location of lysine that are candidates for lysine to arginine amino acid substitutions.

FIG. 36B shows protein expression data in the form of a western blot comparing a BxB1 having a lysine at position 10 substituted for an arginine (K10R) (left panel) versus a BxB1 having a lysine at position 10 (right panel). Samples were collected at 24 and 48 hours.

FIGS. 37A-37B show first attempts at optimizing BxB1 RNA structure using the LinearDesign algorithm. Non-optimized mRNA encoding BxB1 in shown in FIG. 37A with optimized mRNA encoding BxB1 shown in FIG. 37B.

FIGS. 38A-38B show first generations attempts at optimizing an RNA structure for RNA encoding nCas9-RT using the LinearDesign algorithm. Non-optimized mRNA encoding nCas9-RT in shown in FIG. 38A with optimized mRNA encoding nCsa9-RT shown in FIG. 38B.

FIG. 39 ddPCR data for Beacon placement for the nCas9-RT described in FIG. 38A and FIG. 38B.

FIG. 40 shows non-limiting examples of fusion proteins comprising an nCas9-RT are fused with engineered integration enzymes (e.g., BxBT). 5′ UTR=5′ untranslated region; XBG=Xenopus beta globin; NLS=nuclear localization signal; BxBT=BxBT integration enzyme; HA=HA Tag; HiBit=tag; 3′ UTR=3′ untranslated region; 80A's=poly A tail comprising 80 adenines (SEQ ID NO: 721); and Stabilion=stabilion peptide.

FIG. 41 shows non-limiting examples of fusion proteins. Fusion proteins comprising a nCas9 and RT are fused with engineered integration enzymes (e.g., BxBT). 5′ UTR=5′ untranslated region; XBG=Xenopus beta globin; NLS=nuclear localization signal; BxB1=BxB1 integration enzyme; HA=HA Tag; HiBit=tag; 3′ UTR=3′ untranslated region; 80A's=poly A tail comprising 80 adenines (SEQ ID NO: 721); and Stabilion=stabilion peptide.

FIG. 42 shows schematics for non-limiting examples of engineered integrases. 5′ UTR=5′ untranslated region; XBG=Xenopus beta globin; NLS=nuclear localization signal; BxB1=BxB1 integration enzyme; HA=HA Tag; HiBit=tag; 3′ UTR=3′ untranslated region; 80A's=poly A tail comprising 80 adenines (SEQ ID NO: 721); Stabilion=stabilion peptide

FIG. 43 shows ddPCR data for percent (%) PGI in iPSC (clones 52) at day 3 after transduction for each of the conditions indicated on the x-axis.

FIG. 44A shows ddPCR data and flow cytometry data for percent (%) PGI at day 7 for iPSC clone 52 for each of the conditions indicated on the x-axis.

FIG. 44B shows flow cytometry plots of side-scatter versus FITC-A for each of the indicated conditions. FITC-A is an indicator PGI.

FIG. 45 shows ddPCR data for percent (%) PGI in iPSC (clones 52) at day 3 after transduction for each of the conditions indicated on the x-axis.

FIG. 46 shows schematics for non-limiting examples of engineered integrases fused to gene editor polypeptides. 5′ UTR=5′ untranslated region; XBG=Xenopus beta globin; NLS=nuclear localization signal; BxB1=BxB1 integration enzyme; HA=HA Tag; HiBit=tag; 3′ UTR=3′ untranslated region; 80A's=poly A tail comprising 80 adenines (SEQ ID NO: 721); Stabilion=stabilion peptide.

FIG. 47 shows ddPCR data for percent beacon placement in primary human hepatocyte (PHH line HU8412) at day 4 after transduction for each of the conditions indicated on the x-axis. Two different concentrations for each construct were used: 187 fmol and 374 fmol.

FIG. 48 shows ddPCR data for percent beacon placement in primary human hepatocyte (PHH line HU8412) at day 4 after transduction for each of the conditions indicated on the x-axis. PL883 was used as a control with ddPCR data presented in middle panel. Right panel includes a table converting mass (ng) to fmol for the BxB1 mRNA used in these transfections.

FIG. 49 shows ddPCR data for integration data in primary human hepatocyte (PHH line HU8412) at day 4 after transduction for each of the conditions indicated on the x-axis.

FIG. 50 shows ddPCR data for total edit (AttB+AttL) in primary human hepatocyte (PHH line HU8412) at day 4 after transduction for each of the conditions indicated on the x-axis.

FIG. 51 shows ddPCR data for total edit (AttB+AttL) in primary human hepatocyte (PHH line HU8412) at day 4 after transduction for each of the conditions indicated on the x-axis.

FIG. 52 shows ddPCR data for percent beacon placement in primary human hepatocyte (PHH line HU8412) at day 4 after transduction for each of the conditions indicated on the x-axis.

FIG. 53 shows ddPCR data for integration data, total edit (AttB+AttL), and beacon placement in primary human hepatocyte (PHH line HU8412) at day 4 after transduction for each of the conditions indicated on the x-axis. Two different concentrations for each construct were used: 187 fmol and 210 fmol.

FIG. 54A shows a schematic drawing (not to scale) for a working model of Integrase function. Integrase dimers bind to attP and attB and associate the sites to form the complex shown on the left. Site-specific recombination catalyzed by the N-terminal catalytic domains (NTDs) results in the hybrid sites attL and attR (right). The reverse reaction that converts attL and attR to attP and attB does not occur at a measurable rate in the absence of a phage encoded RDF protein. attPL and attPR refer to the left and right half-sites of attP.

FIG. 54B shows domain structure of the Bxb1 LSR, with the amino acid residues demarcating the boundaries of the different domains/motifs indicated. NTD: N-terminal catalytic domain. αE is an alpha helix that extends from the NTD to the recombinase domain (RD). CC: coiled-coil motif embedded in the zinc ribbon domain (ZD).

FIG. 55 shows that certain mutations in the zinc ribbon domain (ZD) maintain activity while improving specificity. The top panel shows that some of the 22 mutations at residues 315-320 of Bxb1 LSR have substantially the same or even higher on-target (on-beacon) integration activity in HEK293 cells compared to that of wild-type Bxb1 LSR, with the horizontal dashed line shows 100% of the relative on-target activity of Bxb1 LSR. The bottom panel shows that all tested mutations have substantially less, or nearly undetectable off-target (off-beacon) integration activity compared to that of wild-type Bxb1 LSR, with the horizontal dashed line shows 100% of the relative off-target activity of Bxb1 LSR. Mutations having substantially the same on-target integration as wt Bxb1 and substantially less (e.g., undetectable) off-target integration compared to wt-Bxb1 (such as the K320E mutation) have higher specificity/fidelity than wt Bxb1 in terms of on-target integration.

FIG. 56 shows similar results as in FIG. 55, in Primary Human Hepatocytes (PHH).

FIG. 57 shows that the K320E mutation increases LSR affinity for on-beacon sites and reduces LSR affinity for off-beacon sites. The data shows that the K320E mutation increased on-beacon affinity by ˜2-fold, and decreased off-beacon affinity by ˜2-5-fold.

FIG. 58 shows the results of scaled off-target evaluation of 8 engineered LSRs. All eLSRs display reduced frequency and scope of off-target edits at 100 top off-target sites. All such so-called HiFi variants eLSRs have improved off target % vs wild type Bxb1 integrase. Decrease in off-target integration is predominantly cryptic site sequence-independent.

FIG. 59 shows sequence alignments of attB sequences for several off-target integration recognition sequences used by Bxb1. Conserved nucleotides are highlighted. Putative contacting nucleotides with the various LSR domains (ZD and RD) are marked as horizontal bars.

FIGS. 60A-60E. Bxb1 binding and activity assays. FIG. 60A is a schematic diagram illustrating the Bxb1 protein has three distinct domains, a N-terminal Domain (NTD) which is responsible for both dimerization and catalysis, a Recombinase Domain (RD) and a Zinc Ribbon Domain (ZD) which contains an extended coiled-coil motif (CC). In solution Bxb1 forms a dimer which binds DNA on either side of a central dinucleotide of either and attB or attP recognition sequence. The CC domains of the attB and attP bound forms interact in the tetramer conformation where a catalytic serine residue cleaves the phosphate backbone of the DNA and forms a covalent bond. Strand exchange then occurs by rotation of the dimers around a planar interface forming two new stable-bound dimers bound to the attL and attR recognition motifs. Finally DNA ligation and dissociation leave an intact double-stranded DNA. FIG. 60B is a schematic diagram illustrating the Bxb1 DNA binding Gel shift assay with the lower band corresponding to unbound and the upper band to bound with a slow migrating species visible in the two highest concentration lanes (left). Curves showing the quantified fraction of Bxb1 bound to DNA as a function of the concentration of enzyme. n=2 error bars are standard deviation (right). FIG. 60C is a schematic diagram illustrating dissociation constants calculated from gel-shift binding assays with error bars representing the 95% confidence interval. FIG. 60D is a schematic diagram illustrating the Bxb1 activity assay. Activity was measured by using DNA substrates containing either attB or attP with the attachment site placed close to 3′ or 5′ end respectively. Prior to recombination the DNA fragments were 180 bp (attB) or 220 bp (attP) and upon recombination form 286 bp (attL) 115 bp (attR), size differences can be seen on the TapeStation gel image (left) and used to calculate a specific activity (right). Error bars represent the standard deviation of n=2 measurements. FIG. 60E is a schematic diagram illustrating specific activity measurements for Bxb1 against DNA substrates containing truncated attachment sites. For each substrate the total length of the fragment was maintained by changing the attachment sequence with A<->C and G<->T. Error bars represent the standard deviation of n=2 measurements.

FIGS. 61A-61E. In vitro activity of WT Bxb1 and zinc ribbon mutants of Bxb1. FIG. 61A is a schematic diagram illustrating the WT attachment site (attB) for Bxb1 (SEQ ID NO: 410). The central dinucleotide is underlined, the recombinase binding region is indicated by a blue bar and the zinc ribbon binding region is indicated by pink bars. FIG. 61B is a schematic diagram illustrating a close up on a mode of the zinc ribbon loop of Bxb1 that has been subject to mutation based on the structure of the LI integrase, positions A315 and K320 are indicated. FIG. 61C is a schematic diagram illustrating the in vitro experiment: Bxb1 (or Bxb1 mutants) are delivered as mRNA, insertion cargo as DNA into HEK293FT cells with a preplaced attB attachment site. FIG. 61D is a schematic diagram illustrating Onbeacon integration activity of the 22 zinc ribbon mutants as measured by ddPCR. FIG. 61E is a schematic diagram illustrating Offbeacon integration activity of the 22 zinc ribbon mutants as measured by ddPCR at CAS031.

FIGS. 62A-62C—In vitro activity of WT Bxb1 and zinc ribbon mutants of Bxb1. FIG. 62A is a schematic diagram illustrating dissociation constants for engineered Bxb1 variants against its native substrates. FIG. 62B is a schematic diagram illustrating dissociation constants for engineered Bxb1 variants against off-beacon substrates. Error bars represent the 95% confidence interval. FIG. 62C s a schematic diagram illustrating specific activity measurements for engineered Bxb1 variants using attB and attP containing substrates. Error bars represent the standard deviation of n=2 measurements. *Upper bounds undetermined for the A315R K320R KD for CAS031.

FIGS. 63A-63G Binding and activity measurements of engineered Bxb1 mutants. FIG. 63A is a schematic diagram illustrating a table of the frequency of a mutation at each amino acid position within Bxb1. FIG. 63B is a schematic diagram illustrating the location of the specific mutations within the 15 tested naturally occurring variants indicated by arrows and amino acid changes on the WT sequence of Bxb1 (SEQ ID NO: 739). The red arrows highlight the constellation of mutations in the zinc ribbon domain that were used to generate variants 12, 13, 14, and 15. FIG. 63C is a schematic diagram illustrating On-beacon integration activity of the 15 naturally occurring mutants as measured by ddPCR. Error bars represent the standard deviation of n=3 measurements. FIG. 63D is a schematic diagram illustrating Off-beacon integration activity of the 15 naturally occurring mutants as measured by ddPCR at CAS031. Error bars represent the standard deviation of n=3 measurements. FIG. 63E is a schematic diagram illustrating dissociation constants for Bxb1 variant 12 against attB and attP. Error bars represent the 95% confidence interval, n=2. FIG. 63F i s a schematic diagram illustrating dissociation constants for Bxb1 variant 12 against off-beacon substrates. Error bars represent the 95% confidence interval, n=2. FIG. 63G is a schematic diagram illustrating specific activity measurements for engineered Bxb1 variant 12 using attB and attP containing substrates. Error bars represent the standard deviation of n=2 measurements.

FIGS. 64A-64F Activity of variants of Bxb1. FIG. 64A is a schematic diagram illustrating the in vivo experiment: On day 0, AVV containing a DNA cargo is delivered via IV. On day 7, Bxb1 (or stabilized Bxb1) is delivered via LNP. On day 14, livers are harvested and genomic DNA is probed for on-beacon integration. FIG. 64B is a schematic diagram illustrating the amino acid sequences of the stabilized Bxb1 molecules. Different stability tags are indicated by variable shadings. FIG. 64C is a schematic diagram illustrating in vivo activity for original stabilized Bxb1 constructs. FIG. 64D is a schematic diagram illustrating in vivo activity for the final panel of stabilized Bxb1 constructs. FIG. 64E is a schematic diagram illustrating in vivo activity for Bxb1 constructs containing both A315R and stabilization tags. FIG. 64F is a schematic diagram illustrating fold increase of in vivo activity of Bxb1 constructs compared to Bxb1 WT-1.

FIG. 65A shows the size exclusion chromatography trace of Bxb1 2-500 showing its observed retention time co-elutes with a 158 kDa molecular weight standard. FIG. 65B show recombinase activity assay curves for minimal attB sequence determination with the WT attP sequence. Error bars represent standard deviation n=2.

FIG. 66A shows binding assay curves for Bxb1 (wild-type), for attB, attP, attL, attR, and CAS031 and CAS421 off-beacon sequences. Error bars represent standard deviation n=2. FIGS. 66B-66J show binding assay curves for Bxb1 variants, for attB, attP, CAS031, and CAS421 off-beacon sequences. Error bars represent standard deviation n=2.

FIGS. 67A-67J show recombinase activity assay curves for Bxb1 (wild-type) and Bxb1 variants, for attB and attP. Error bars represent standard deviation n=2.

### 6. DETAILED DESCRIPTION

The invention described herein is partly based on the discovery that potential off-target integration sites (sometimes referred to herein as “off-beacon sites”) are more conserved in the recombinase binding region than in the other domains such as the zinc ribbon domain (ZD). See FIG. 59. Thus, mutations in, for example, the zinc ribbon domain (ZD) of the Integrases (also used herein interchangeably with “engineered large serine integrase,” “eLSR,” or “integration enzyme”) could reduce off-beacon integration without significantly affecting on-target/on-beacon integration.

More specifically, invention described herein provides engineered LSRs having mutations (such as mutations in the zinc ribbon domain) that maintain integrase activity while improving specificity/fidelity of cargo/donor DNA integration at the intended target integration sites having “beacons,” while substantially reducing or eliminating off-target integration at unintended/undesirable non-target (off-beacon) genomic locations.

Therefore, described herein are integration enzymes (e.g., lareg serine integrase or LSR) engineered such that upon being introduced into a cell, the integration enzyme has enhanced fidelity/specificity for target sites having matching cognate integration recognition sites. The engineered large serine integrase (eLSR) comprises one or more substitutions that substantially maintain or enhance integration activity at a pair of cognate integration recognition sites, and substantially decrease off-target integration activity at a pair of off-target integration recognition sites, when compared to a corresponding large serine integrase without said one or more substitutions (cLSR). In certain embodiments, the one or more substitutions are in a zinc ribbon domain (ZD) of the cLSR.

In certain embodiments, the eLSR is engineered such that upon being introduced into a cell, it has increased stability (e.g., half-life) compared to a control cLSR not engineered to have increased stability. The increase in stability extends the capacity of the eLSR to mediate integration. For example, this disclosure features an eLSR engineered to include a stabilization domain on the N-terminus, which resulted in increased integration of a donor polynucleotide template into an integration recognition site (i.e., beacon) placed into a genome of a cell as compared to a corresponding or control integration enzyme not engineered to include the stabilization domain. In typical embodiments, the engineered integration enzyme (eLSR) includes a mutation that enhances fidelity of the integration enzyme or fragment thereof, optionally an at least first stabilization domain, and further optionally a nuclear localization signal. At least in some case, location (e.g., N-terminus or C-terminus) of the stabilization domain (when present) in the eLSR impacted the integration enzyme's ability to mediate integration.

### 6.1. Terminology

Unless defined otherwise, all technical and scientific terms used herein have the meaning commonly understood by a person skilled in the art to which this invention belongs. As used herein, the following terms have the meanings ascribed to them below.

“Gene editor” as used herein, is a protein that that can be used to perform gene editing, gene modification, gene insertion, gene deletion, or gene inversion. As used herein, the terms “gene editor polynucleotide” refers to polynucleotide sequence encoding the gene editor protein. Such an enzyme or enzyme fusion may contain DNA or RNA targetable nuclease protein (i.e., Cas protein, ADAR, or ADAT), wherein target specificity is mediated by a complexed nucleic acid (i.e., guide RNA). Such an enzyme or enzyme fusion may be a DNA/RNA targetable protein, wherein target specificity is mediated by internal, conjugated, fused, or linked amino acids, such as within TALENs, ZFNs, or meganucleases. The skilled person in the art would appreciate that the gene editor can demonstrate targeted nuclease activity, targeted binding with no nuclease activity, or targeted nickase activity (or cleavase activity). A gene editor comprising a targetable protein may be fused, linked, complexed, operate in cis or trans to one or more proteins or protein fragment motifs. Gene editors may be fused or linked to one or more integrase, recombinase, polymerase, telomerase, reverse transcriptase, or invertase. A gene editor can be a prime editor fusion protein or a gene writer fusion protein.

“Alpha editor fusion protein” or “Prime editor fusion protein” as used herein interchangeably, describes a protein that is used in prime editing (also used herein for “alpha editing”). “Alpha editor system” or “Prime editor system” as used interchangeably herein describes the components used in alpha editing/prime editing. Alpha editing/prime editing uses CRISPR enzyme that nicks or cuts only single strand of double stranded DNA, i.e., a nickase; the nickase can occur either naturally or by mutation or modification of a nuclease that makes double stranded cuts. The nickase is programmed (directed) with a prime-editing guide RNA (pegRNA). The skilled person in the art would appreciate that the pegRNA both specifies the target site and encodes the desired edit. Described herein are attachment site containing guide RNA (atgRNA) that both specifies the target and encodes for the desired integrase target recognition site. The nickase may be programmed (directed) with an atgRNA. Advantageously the nickase is a catalytically impaired Cas9 endonuclease, a Cas9 nickase, that is fused to the reverse transcriptase. During genetic editing, the Cas9 nickase part of the protein is guided to the DNA target site by the atgRNA (or pegRNA), whereby a nick or single stranded cut occurs. The reverse transcriptase domain then uses the atgRNA (or pegRNA) to template reverse transcription of the desired edit, directly polymerizing DNA onto the nicked target DNA strand. The edited DNA strand replaces the original DNA strand, creating a heteroduplex containing one edited strand and one unedited strand. Afterward, optionally, the prime editor (PE) guides resolution of the heteroduplex to favor copying the edit onto the unedited strand, completing the process (typically achieved with a nickase gRNA). Other enzymes that can be used to nick or cut only a single strand of double stranded DNA includes a cleavase (e.g., cleavase I enzyme).

In some embodiments, an additional agent or agents may be added that improve the efficiency and outcome purity of the prime edit. In some embodiments, the agent may be chemical or biological and disrupt DNA mismatch repair (MMR) processes at or near the edit site (i.e., PE4 and PE5 and PEmax architecture by Chen et al. Cell, 184, 1-18, Oct. 28, 2021; Chen et al. is incorporated herein by reference). In typical embodiments, the agent is a MMR-inhibiting protein. In certain embodiments, the MMR-inhibiting protein is dominant negative MMR protein. In certain embodiments, the dominant negative MMR protein is MLH1dn. In particular embodiments, the MMR-inhibiting agent is incorporated into the co-delivery method described herein. In some embodiments, the MMR-inhibiting agent is linked or fused to the prime editor protein fusion, which may or may not have a linked or fused integrase. In some embodiments, the MMR-inhibiting agent is linked or fused to the Gene Writer™ protein, which may or may not have a linked or fused integrase.

The prime editor or gene editor system can be used to achieve DNA deletion and replacement. In some embodiments, the DNA deletion replacement is induced using a pair of atgRNAs or pegRNA that target opposite DNA strands, programming not only the sites that are nicked but also the outcome of the repair (i.e., PrimeDel by Choi et al. Nat. Biotechnology, Oct. 14, 2021; Choi et al. is incorporated herein by reference and TwinPE by Anzalone et al. BioRxiv, Nov. 2, 2021; Anzalone et al. is incorporated herein by reference). In some embodiments described herein, the DNA deletion is induced using a single atgRNA. In some embodiments, the DNA deletion and replacement is induced using a wild type Cas9 prime editor (PE-Cas9) system (i.e., PEDAR by Jiang et al. Nat. Biotechnology, Oct. 14, 2021; Jiang et al. is incorporated herein by reference in its entirety). In some embodiments, the DNA replacement is an integrase target recognition site or recombinase target recognition site. In certain embodiments, the constructs and methods described herein may be utilized to incorporate the pair of pegRNAs (or atgRNAs) used in PrimeDel, TwinPE (WO2021226558 incorporated by reference herein in its entirety), or PEDAR, the prime editor fusion protein or Gene Writer protein, optionally a nickase guide RNA (ngRNA), an integrase, a nucleic acid cargo, and optionally a recombinase into a LNP delivery system or vector delivery system (e.g., AAV or Adenovirus). The integrase may be directly linked, for example by a peptide linker, to the prime editor fusion or gene writer protein.

In some embodiments, the prime editors can refer to a retrovirus or lentivirus reverse transcriptase such as a Moloney Murine Leukemia Virus (M-MLV) reverse transcriptase (RT) fused to a CRISPR enzyme nickase such as a Cas9 H840A nickase, a Cas9nickase. In some embodiments, the prime editors can refer to a retrovirus or lentivirus reverse transcriptase such as a Moloney Murine Leukemia Virus (M-MLV) reverse transcriptase (RT) fused to a cleavase. In some embodiments the RT can be fused at, near or to the C-terminus of a Cas9nickase, e.g., Cas9 H840A. Fusing the RT to the C-terminus region, e.g., to the C-terminus, of the Cas9 nickase may result in higher editing efficiency. Such a complex is called PEI. In some embodiments, the CRISPR enzyme nickase, e.g., Cas9(H840A), i.e., a Cas9nickase, can be linked to a non-M-MLV reverse transcriptase such as an AMV-RT or XRT (Cas9(H840A)-AMV-RT or XRT). In some embodiments, instead of the CRISPR enzyme nickase being a Cas9 (H840A), i.e., instead of being a Cas9 nickase, the CRISPR enzyme nickase instead can be a CRISPR enzyme that naturally is a nickase or cuts a single strand of double stranded DNA; for instance, the CRISPR enzyme nickase can be Cas12a/b. Alternatively, the CRISPR enzyme nickase can be another mutation of Cas9, such as Cas9(Dl0A). A CRISPR enzyme, such as a CRISPR enzyme nickase, such as Cas9 (wild type), Cas9(H840A), Cas9(Dl0A) or Cas 12a/b nickase can be fused in some embodiments to a pentamutant of M-MLV RT (D200N/L603W/T330P/T306K/W313F), whereby there can be up to about 45-fold higher efficiency, and this is called PE2. In some embodiments, the M-MLV RT comprise one or more of the mutations Y8H, P51L, S56A, S67R, E69K, V129P, L139P, T197A, H204R, V223H, T246E, N249D, E286R, Q2911, E302K, E302R, F309N, M320L, P330E, L435G, L435R, N454K, D524A, D524G, D524N, E562Q, D583N, H594Q, E607K, D653N, and L671P. Specific M-MLV RT mutations are shown in Table 1.

TABLE 1

| | | Forward Sequence |
|---|---|---|
| SEQ ID NO | Description | (5′-3′) |
| SEQ ID NO: 01 | RT mut L139P | ttgagcgggC |
| | | CCccaccgt |
| SEQ ID NO: 02 | RT mut E562Q | cagcgggctC |
| | | AGctgatagca |
| SEQ ID NO: 03 | RT mut D653N | cggatggctA |
| | | ACcaagcggcc |

In some embodiments, the reverse transcriptase can also be a wild-type or modified transcription xenopolymerase (RTX), avian myeloblastosis virus reverse transcriptase (AMV RT), Feline Immunodeficiency Virus reverse transcriptase (FIV-RT), FeLV-RT (Feline leukemia virus reverse transcriptase), HIV-RT (Human Immunodeficiency Virus reverse transcriptase). In some embodiments, the reverse transcriptase can be a fusion of MMuLV to the Sto7d DNA binding domain (see lonnidi et al.; https://doi.org/10.1101/2021.11.01.466786). The fusion of MMuLV to the Sto7d DNA binding domain sequence is given in Table 2.

TABLE 2

| | | SEQ |
|---|---|---|
| | | ID |
| Description | Forward Sequence (5′-3′) | NO: |
| RT(1- | atgactcactatcaggccttgct | 4 |
| 478)_ | tttggacacggaccgggtccag | |
| Sto7d | ttcggaccggtggtagccctga | |
| fusion | acccggctacgctgctcccact | |
| [MMulv | gcctgaggaagggctgcaacac | |
| sequence | aactgccttgatGGGACAGGTG | |
| (in | GCGGTGGTGTCACCGTCAAGTT | |
| bold), | CAAGTACAAGGGTGAGGAACTT | |
| Sto7d | GAAGTTGATATTAGCAAAATCA | |
| sequence] | AGAAGGTTTGGCGCGTTGGTAA | |
| | AATGATATCTTTTACTTATGAC | |
| | GACAACGGCAAGACAGGTAGAG | |
| | GGGCAGTGTCTGAGAAAGACGC | |
| | CCCCAAGGAGCTGTTGCAAATG | |
| | TTGGAAAAGTCTGGGAAAAAGt | |
| | ctggcggctcaaaaagaaccgc | |
| | cgacggcagegaattcgagccc | |
| | aagaagaagaggaaagtc | |

PE3, PE3b, PE4, PE5, and/or PEmax, which a skilled person can incorporate into the co-delivery system described herein, involves nicking the non-edited strand, potentially causing the cell to remake that strand using the edited strand as the template to induce HR. The nicking of the non-edited strand can involve the use of a nicking guide RNA (ngRNA).

The skilled person can readily incorporate into the co-delivery system described herein described herein a prime editing or CRISPR system. Examples of prime editors can be found in the following: WO2020/191153, WO2020/191171, WO2020/191233, WO2020/191234, WO2020/191239, WO2020/191241, WO2020/191242, WO2020/191243, WO2020/191245, WO2020/191246, WO2020/191248, WO2020/191249, each of which is incorporated by reference herein in its entirety. In addition, mention is made, and can be used herein, of CRISPR Patent Applications and Patents of the Zhang laboratory and/or Broad Institute, Inc. and Massachusetts Institute of Technology and/or Broad Institute, Inc., Massachusetts Institute of Technology and President and Fellows of Harvard College and/or Editas Medicine, Inc. Broad Institute, Inc., The University of Iowa Research Foundation and Massachusetts Institute of Technology, including those claiming priority to U.S. Application 61/736,527, filed Dec. 12, 2012, including U.S. Pat. Nos. 11,104,937, 11,091,798, 11,060,115, 11,041,173, 11,021,740, 11,008,588, 11,001,829, 10,968,257, 10,954,514, 10,946,108, 10,930,367, 10,876,100, 10,851,357, 10,781,444, 10,711,285, 10,689,691, 10,648,020, 10,640,788, 10,577,630, 10,550,372, 10,494,621, 10,377,998, 10,266,887, 10,266,886, 10,190,137, 9,840,713, 9,822,372, 9,790,490, 8,999,641, 8,993,233, 8,945,839, 8,932,814, 8,906,616, 8,895,308, 8,889,418, 8,889,356, 8,871,445, 8,865,406, 8,795,965, 8,771,945, and 8,697,359; CRISPR Patent Applications and Patents of the Doudna laboratory and/or of Regents of the University of California, the University of Vienna and Emmanuelle Charpentier, including those claiming priority to U.S. application 61/652,086, filed May 25, 2012, and/or 61/716,256, filed Oct. 19, 2012, and/or 61/757,640, filed Jan. 28, 2013, and/or 61/765,576, filed Feb. 15, 2013 and/or 13/842,859, including U.S. Pat. Nos. 11,028,412, 11,008,590, 11,008,589, 11,001,863, 10,988,782, 10,988,780, 10,982,231, 10,982,230, 10,900,054, 10,793,878, 10,774,344, 10,752,920, 10,676,759, 10,669,560, 10,640,791, 10,626,419, 10,612,045, 10,597,680, 10,577,631, 10,570,419, 10,563,227, 10,550,407, 10,533,190, 10,526,619, 10,519,467, 10,513,712, 10,487,341, 10,443,076, 10,428,352, 10,421,980, 10,415,061, 10,407,697, 10,400,253, 10,385,360, 10,358,659, 10,358,658, 10,351,878, 10,337,029, 10,308,961, 10,301,651, 10,266,850, 10,227,611, 10,113,167, and 10,000,772; CRISPR Patent Applications and Patents of Vilnius University and/or the Siksnys laboratory, including those claiming priority to U.S. application 62/046,384 and/or 61/625,420 and/or 61/613,373 and/or PCT/IB2015/056756, including U.S. Pat. No. 10,385,336; CRISPR Patent Applications and Patents of the President and Fellows of Harvard College, including those of George Church's laboratory and/or claiming priority to U.S. application 61/738,355, filed Dec. 17, 2012, including 11,111,521, 11,085,072, 11,064,684, 10,959,413, 10,925,263, 10,851,369, 10,787,684, 10,767,194, 10,717,990, 10,683,490, 10,640,789, 10,563,225, 10,435,708, 10,435,679, 10,375,938, 10,329,587, 10,273,501, 10,100,291, 9,970,024, 9,914,939, 9,777,262, 9,587,252, 9,267,135, 9,260,723, 9,074,199, 9,023,649; CRISPR Patent Applications and Patents of the President and Fellows of Harvard College, including those of David Liu's laboratory, including 11,111,472, 11,104,967, 11,078,469, 11,071,790, 11,053,481, 11,046,948, 10,954,548, 10,947,530, 10,912,833, 10,858,639, 10,745,677, 10,704,062, 10,682,410, 10,612,011, 10,597,679, 10,508,298, 10,465,176, 10,323,236, 10,227,581, 10,167,457, 10,113,163, 10,077,453, 9,999,671, 9,840,699, 9,737,604, 9,526,784, 9,388,430, 9,359,599, 9,340,800, 9,340,799, 9,322,037, 9,322,006, 9,228,207, 9,163,284, and 9,068,179; and CRISPR Patent Applications and Patents of Toolgen Incorporated and/or the Kim laboratory and/or claiming priority to U.S. application 61/717,324, filed Oct. 23, 2012 and/or 61/803,599, filed Mar. 20, 2013 and/or 61/837,481, filed Jun. 20, 2013 and/or 62/033,852, filed Aug. 6, 2014 and/or PCT/KR2013/009488 and/or PCT/KR2015/008269, including U.S. Pat. Nos. 10,851,380, and 10,519,454; and CRISPR Patent Applications and Patents of Sigma and/or Millipore and/or the Chen laboratory and/or claiming priority to U.S. application 61/734,256, filed Dec. 6, 2012 and/or 61/758,624, filed Jan. 30, 2013 and/or 61/761,046, filed Feb. 5, 2013 and/or 61/794,422, filed Mar. 15, 2013, including U.S. Pat. No. 10,731,181, each of which is hereby incorporated herein by reference, and from the disclosures of the foregoing, the skilled person can readily make and use a prime editing or CRISPR system, and can especially appreciate impaired endonucleases, such as a mutated Cas9 that only nicks a single strand of DNA and is hence a nickase, or a CRISPR enzyme that only makes a single-stranded cut that can be employed in a PASTE system of the invention. Further, from the disclosures of the foregoing, the skilled person can incorporate the selected CRISPR enzyme, as part of the prime editor fusion or gene editor fusion, into the co-delivery method described herein.

Prior to RT-mediated edit incorporation, the prime editor protein (or system) (1) site-specifically targets a genomic locus and (2) performs a catalytic cut or nick. These steps are typically performed by a CRISPR-Cas. However, in some embodiments the Cas protein may be substituted by other nucleic acid programmable DNA binding proteins (napDNAbp) such as zinc finger nucleases (ZFNs), transcription activator-like effector nucleases (TALENs), or meganucleases. In addition, to the extent the “targeting rules” of other napDNAbp are known or are newly determined, it becomes possible to use new napDNAbp, beyond Cas9, to site specifically target and modify genomic sites of interest.

Similar to a prime editor protein, a Gene Writer can introduce novel DNA elements, such as an integration target site, into a DNA locus. A Gene Writer protein comprises: (A) a polypeptide or a nucleic acid encoding a polypeptide, wherein the polypeptide comprises (i) a reverse transcriptase domain, and either (x) an endonuclease domain that contains DNA binding functionality or (y) an endonuclease domain and separate DNA binding domain; and (B) a template RNA comprising (i) a sequence that binds the polypeptide and (ii) a heterologous insert sequence. Examples of such Gene Writer™ proteins and related systems can be found in US20200109398, which is incorporated by reference herein in its entirety.

In some embodiments, the prime editor or Gene Writer protein fusion or prime editor protein linked or fused to an integrase is expressed as a split construct. In typical embodiments, the split construct in reconstituted in a cell. In some embodiments, the split construct can be fused or ligated via intein protein splicing. In some embodiments, the split construct can be reconstituted via protein-protein inter-molecular bonding and/or interactions. In some embodiments, the split construct can be reconstituted via chemical, biological, or environmental induced oligomerization. In certain embodiments, the split construct can be adapted into one or more delivery vectors described herein.

In some embodiments, an integrase or recombinase is directly linked or fused, for example by a peptide linker, which may be cleavable or non-cleavable, to the prime editor fusion protein (i.e., fused Cas9 nickase-reverse transcriptase) or Gene Writer protein. Suitable linkers, for example between the Cas9, RT, and integrase, may be selected from Table 3:

TABLE 3

| | | SEQ ID | | SEQ ID |
|---|---|---|---|---|
| | Sequence (5′-3′) | NO: | Amino acid sequence | NO: |
| A - P2A | GGAAGCGGAGCTACTAACTTCAGCCT | 15 | GSGATNFSLLKQAGDVE | 13 |
| | GCTGAAGCAGGCTGGCGACGTGGAG | | ENPGP | |
| | GAGAACCCTGGACCT | | | |
| B - | GGGGGAGGAGGTTCTGGAGGCGGAG | 6 | GGGGSGGGGSGGGGS | 14 |
| (GGGS)3 | GCTCCGGAGGCGGAGGGTCA | | | |
| (SEQ ID | | | | |
| NO: 678) | | | | |
| C - | GGAGGTGGCGGGAGC | 7 | GGGGS | 15 |
| GGGGS | | | | |
| (SEQ ID | | | | |
| NO: 15) | | | | |
| D - PAPAP | CCCGCACCAGCGCCT | 8 | PAPAP | 16 |
| (SEQ ID | | | | |
| No: 16) | | | | |
| E - | GAGGCAGCTGCCAAGGAAGCCGCTG | | EAAAKEAAAKEAAAK | 17 |
| (EAAAK)3 | CCAAGGAGGCGGCCGCAAAG | | | |
| (SEQ ID | | | | |
| NO: 17) | | | | |
| F - XTEN | AGTGGGAGCGAGACCCCTGGGACTA | 10 | SGSETPGTSESATPES | 18 |
| | GCGAGTCAGCTACACCCGAAAGC | | | |
| G - (GGS)6 | GGGGGGTCAGGTGGATCCGGCGGAA | 11 | GGSGGSGGSGGSGGSG | 19 |
| (SEQ ID | GTGGCGGATCCGGTGGATCTGGCGG | | GS | |
| NO: 19) | CAGT | | | |
| H - EAAAK | GAAGCTGCTGCTAAG | 12 | EAAAK | 20 |
| (SEQ ID | | | | |
| NO: 20) | | | | |
| (GGGGS)4( | GGCGGCGGCGGCAGCGGCGGCGGC | 543 | GGGGSGGGGSGGGGS | 551 |
| SEQ ID NO: | GGCAGCGGCGGCGGCGGCAGCGGC | | GGGGS | |
| 551) | GGCGGCGGCAGC | | | |
| PAS8 | GGCGGCGCGAGCCCGGCGGGCGGC | 544 | GGASPAGG | 552 |
| PAS12 | GGCGGCGCGAGCCCGGCGGCGCCG | 545 | GGASPAAPAPAG | 553 |
| | GCGCCGGCGGGC | | | |
| A(EAAK)4A | GCGGAAGCGGCGAAAGAAGCGGCGA | 546 | AEAAKEAAKEAAKEAAKA | 554 |
| LEA(EAAA | AAGAAGCGGCGAAAGAAGCGGCGAA | | LEAEAAAKEAAAKEAAAK | |
| K)4A (SEQ | AGCGCTGGAAGCGGAAGCGGCGGCG | | EAAAKA | |
| ID NO: 554) | AAAGAAGCGGCGGCGAAAGAAGCGG | | | |
| | CGGCGAAAGAAGCGGCGGCGAAAGC | | | |
| | G | | | |
| Camel | GCGCATCATAGCGAAGATCCGGGCG | 547 | AHHSEDPGGGGSGGGG | 555 |
| | GCGGCGGCAGCGGCGGCGGCGGCA | | SGGGGS | |
| | GCGGCGGCGGCGGCAGC | | | |
| FRF | GGCGGCGGCGGCAGCGAAGCGGCG | 548 | GGGGSEAAAKGGGGS | 556 |
| | GCGAAAGGCGGCGGCGGCAGC | | | |
| RFF | GAAGCGGCGGCGAAAGGCGGCGGC | 549 | EAAAKGGGGSEAAAK | 557 |
| | GGCAGCGAAGCGGCGGCGAAA | | | |
| Modified | AGCGGCGGCAGCAGCGGCGGCAGCA | 550 | SGGSSGGSSGSETPGTS | 558 |
| XTEN | GCGGCAGCGAAACCCCGGGCACCAG | | ESATPESSGGSSGGSST | |
| (mXTEN) | CGAAAGCGCGACCCCGGAAAGCAGC | | | |
| | GGCGGCAGCAGCGGCGGCAGCAGCA | | | |
| | CC | | | |

In some embodiments, the prime editor or Gene Writer protein fusion or prime editor protein linked or fused to an integrase is expressed as a split construct. In typical embodiments, the split construct in reconstituted in a cell. In some embodiments, the split construct can be fused or ligated via intein protein splicing. In some embodiments, the split construct can be reconstituted via protein-protein inter-molecular bonding and/or interactions. In some embodiments, the split construct can be reconstituted via chemical, biological, or environmental induced oligomerization. In certain embodiments, the split construct can be adapted into one or more nucleic acid constructs described herein.

### 6.2. Type II CRISPR Proteins

The skilled person can incorporate a selected CRISPR enzyme, described below, as part of the prime editor fusion, into the co-delivery method described herein. Streptococcus pyogenes Cas9 (SpCas9), the most common enzyme used in genome-editing applications, is a large nuclease of 1368 amino acid residues. Advantages of SpCas9 include its short, 5′-NGG-3′ PAM and very high average editing efficiency. SpCas9 consists of two lobes: a recognition (REC) lobe and a nuclease (NUC) lobe. The REC lobe can be divided into three regions, a long a helix referred to as the bridge helix (residues 60-93), the REC1 (residues 94-179 and 308-713) domain, and the REC2 (residues 180-307) domain. The NUC lobe consists of the RuvC (residues 1-59, 718-769, and 909-1098), HNH (residues 775-908), and PAM-interacting (PI) (residues 1099-1368) domains. The negatively charged sgRNA:target DNA heteroduplex is accommodated in a positively charged groove at the interface between the REC and NUC lobes. In the NUC lobe, the RuvC domain is assembled from the three split RuvC motifs (RuvC I-III) and interfaces with the PI domain to form a positively charged surface that interacts with the 30 tail of the sgRNA. The HNH domain lies between the RuvC II-III motifs and forms only a few contacts with the rest of the protein. Structural aspects of SpCas9 are described by Nishimasu et al., Crystal Structure of Cas9 in Complex with Guide RNA and Target DNA, Cell 156, 935-949, Feb. 27, 2014.

REC lobe: The REC lobe includes the REC1 and REC2 domains. The REC2 domain does not contact the bound guide:target heteroduplex, indicating that truncation of REC lobe may be tolerated by SpCas9. Further, SpCas9 mutant lacking the REC2 domain (D175-307) retained ˜50% of the wild-type Cas9 activity, indicating that the REC2 domain is not critical for DNA cleavage. In striking contrast, the deletion of either the repeat-interacting region (D97-150) or the anti-repeat-interacting region (D312-409) of the REC1 domain abolished the DNA cleavage activity, indicating that the recognition of the repeat:anti-repeat duplex by the REC1 domain is critical for the Cas9 function.

PAM-Interacting domain: The NUC lobe contains the PAM-interacting (PI) domain that is positioned to recognize the PAM sequence on the noncomplementary DNA strand. The PI domain of SpCas9 is required for the recognition of 5′-NGG-3′ PAM, and deletion of the PI domain (A1099-1368) abolished the cleavage activity, indicating that the PI domain is critical for SpCas9 function and a major determinant for the PAM specificity.

RuvC domain: The RuvC nucleases of SpCas9 have an RNase H fold and four catalytic residues, Asp10 (Ala), Glu762, His983, and Asp986, that are critical for the two-metal cleavage of the noncomplementary strand of the target DNA. In addition to the conserved RNase H fold, the Cas9 RuvC domain has other structural elements involved in interactions with the guide:target heteroduplex (an end-capping loop between α42 and α43) and the PI domain/stem loop 3 (B hairpin formed by β3 and β4).

HNH domain: SpCas9 HNH nucleases have three catalytic residues, Asp839, His840, and Asn863 and cleave the complementary strand of the target DNA through a single-metal mechanism.

sgRNA:DNA recognition: The sgRNA guide region is primarily recognized by the REC lobe. The backbone phosphate groups of the guide region (nucleotides 2, 4-6, and 13-20) interact with the REC1 domain (Arg165, Gly166, Arg403, Asn407, Lys510, Tyr515, and Arg661) and the bridge helix (Arg63, Arg66, Arg70, Arg71, Arg74, and Arg78). The 20-hydroxyl groups of G1, C15, U16, and G19 hydrogen bond with Val1009, Tyr450, Arg447/Ile448, and Thr404, respectively.

A mutational analysis demonstrated that the R66A, R70A, and R74A mutations on the bridge helix markedly reduced the DNA cleavage activities, highlighting the functional significance of the recognition of the sgRNA “seed” region by the bridge helix. Although Arg78 and Arg165 also interact with the “seed” region, the R78A and R165A mutants showed only moderately decreased activities. These results are consistent with the fact that Arg66, Arg70, and Arg74 form multiple salt bridges with the sgRNA backbone, whereas Arg78 and Arg165 form a single salt bridge with the sgRNA backbone. Moreover, the alanine mutations of the repeat:anti-repeat duplex-interacting residues (Arg75 and Lys163) and the stemloop-1-interacting residue (Arg69) resulted in decreased DNA cleavage activity, confirming the functional importance of the recognition of the repeat:anti-repeat duplex and stem loop 1 by Cas9.

RNA-guided DNA targeting: SpCas9 recognizes the guide:target heteroduplex in a sequence-independent manner. The backbone phosphate groups of the target DNA (nucleotides 1, 9-11, 13, and 20) interact with the REC1 (Asn497, Trp659, Arg661, and Gln695), RuvC (Gln926), and PI (Glu1108) domains. The C2′ atoms of the target DNA (nucleotides 5, 7, 8, 11, 19, and 20) form van der Waals interactions with the REC1 domain (Leu169, Tyr450, Met495, Met694, and His698) and the RuvC domain (Ala728). The terminal base pair of the guide:target heteroduplex (G1:C20′) is recognized by the RuvC domain via end-capping interactions; the sgRNA G1 and target DNA C20′ nucleobases interact with the Tyr1013 and Val1015 side chains, respectively, whereas the 20-hydroxyl and phosphate groups of sgRNA G1 interact with Val1009 and Gln926, respectively.

Repeat: Anti-Repeat duplex recognition: The nucleobases of U23/A49 and A42/G43 hydrogen bond with the side chain of Arg1122 and the main-chain carbonyl group of Phe351, respectively. The nucleobase of the flipped U44 is sandwiched between Tyr325 and His328, with its N3 atom hydrogen bonded with Tyr325, whereas the nucleobase of the unpaired G43 stacks with Tyr359 and hydrogen bonds with Asp364.

The nucleobases of G21 and U50 in the G21:U50 wobble pair stack with the terminal C20:G10 pair in the guide:target heteroduplex and Tyr72 on the bridge helix, respectively, with the U50 O4 atom hydrogen bonded with Arg75. Notably, A51 adopts the syn conformation and is oriented in the direction opposite to U50. The nucleobase of A51 is sandwiched between Phe1105 and U63, with its N1, N6, and N7 atoms hydrogen bonded with G62, Gly1103, and Phe1105, respectively.

Stem-loop recognition: Stem loop 1 is primarily recognized by the REC lobe, together with the PI domain. The backbone phosphate groups of stem loop 1 (nucleotides 52, 53, and 59-61) interact with the REC1 domain (Leu455, Ser460, Arg467, Thr472, and I1e473), the PI domain (Lys1123 and Lys1124), and the bridge helix (Arg70 and Arg74), with the 20-hydroxyl group of G58 hydrogen bonded with Leu455. A52 interacts with Phe1105 through a face-to-edge p-p stacking interaction, and the flipped U59 nucleobase hydrogen bonds with Asn77.

The single-stranded linker and stem loops 2 and 3 are primarily recognized by the NUC lobe. The backbone phosphate groups of the linker (nucleotides 63-65 and 67) interact with the RuvC domain (Glu57, Lys742, and Lys1097), the PI domain (Thr1102), and the bridge helix (Arg69), with the 20-hydroxyl groups of U64 and A65 hydrogen bonded with Glu57 and His721, respectively. The C67 nucleobase forms two hydrogen bonds with Val1100.

Stem loop 2 is recognized by Cas9 via the interactions between the NUC lobe and the non-Watson-Crick A68:G81 pair, which is formed by direct (between the A68 N6 and G81 O6 atoms) and water-mediated (between the A68 N1 and G81 N1 atoms) hydrogen-bonding interactions. The A68 and G81 nucleobases contact Ser1351 and Tyr1356, respectively, whereas the A68:G81 pair interacts with Thr1358 via a water-mediated hydrogen bond. The 20-hydroxyl group of A68 hydrogen bonds with His1349, whereas the G81 nucleobase hydrogen bonds with Lys33.

Stem loop 3 interacts with the NUC lobe more extensively, as compared to stem loop 2. The backbone phosphate group of G92 interacts with the RuvC domain (Arg40 and Lys44), whereas the G89 and U90 nucleobases hydrogen bond with Gln1272 and Glu1225/Ala1227, respectively. The A88 and C91 nucleobases are recognized by Asn46 via multiple hydrogen-bonding interactions.

Cas9 proteins smaller than SpCas9 allow more efficient packaging of nucleic acids encoding CRISPR systems, e.g., Cas9 and sgRNA into one rAAV (“all-in-one-AAV”) particle. In addition, efficient packaging of CRISPR systems can be achieved in other viral vector systems (i.e., lentiviral, integration deficient lentiviral, hd-AAV, etc.) and non-viral vector systems (i.e., lipid nanoparticle). Small Cas9 proteins can be advantageous for multidomain-Cas-nuclease-based systems for prime editing. Well characterized smaller Cas9 proteins include Staphylococcus aureus (SauCas9, 1053 amino acid residues) and Campylobacter jejuni (CjCas9, 984 amino residues). However, both recognize longer PAMs, 5′-NNGRRT-3′ for SauCas9 (R=A or G) and 5′-NNNNRYAC-3′ for CjCas9 (Y=C or T), which reduces the number of uniquely addressable target sites in the genome, in comparison to the NGG SpCas9 PAM. Among smaller Cas9s, Schmidt et al. identified Staphylococcus lugdunensis (Slu) Cas9 as having genome-editing activity and provided homology mapping to SpCas9 and SauCas9 to facilitate generation of nickases and inactive (“dead”) enzymes (Schmidt et al., 2021, Improved CRISPR genome editing using small highly active and specific engineered RNA-guided nucleases. Nat Commun 12, 4219. doi.org/10.1038/s41467-021-24454-5) and engineered nucleases with higher cleavage activity by fragmenting and shuffling Cas9 DNAs. The small Cas9s and nickases are useful in the instant invention.

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 18).

In some embodiments, the disclosure also may utilize Cas9 fragments that retain their functionality and that are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the prime editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

TABLE 4
Cas9 orthologs

| | | |
|---|---|---|
| Streptococcus | MDKKYSIGLD IGTNSVGWAV ITDEYKVPSK KFKVLGNTDR HSIKKNLIGA | (SEQ |
| pyogenes | LLFDSGETAE ATRLKRTARR RYTRRKNRIC YLQEIFSNEM AKVDDSFFHR | ID NO: |
| AJN60024.1 | LEESFLVEED KKHERHPIFG NIVDEVAYHE KYPTIYHLRK KLVDSTDKAD | 21) |
| GI: 757015980 | LRLIYLALAH MIKFRGHFLI EGDLNPDNSD VDKLFIQLVQ TYNQLFEENP | |
| WP_01092225 | INASGVDAKA ILSARLSKSR RLENLIAQLP GEKKNGLFGN LIALSLGLTP | |
| 1.1 | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNFDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRFNAS LGTYHDLLKI | |
| | IKDKDFLDNE ENEDILEDIV LTLTLFEDRE MIEERLKTYA HLFDDKVMKQ | |
| | LKRRRYTGWG RLSRKLINGI RDKQSGKTIL DFLKSDGFAN RNFMQLIHDD | |
| | SLTFKEDIQK AQVSGQGDSL HEHIANLAGS PAIKKGILQT VKVVDELVKV | |
| | MGRHKPENIV IEMARENQTT QKGQKNSRER MKRIEEGIKE LGSQILKEHP | |
| | VENTQLQNEK LYLYYLQNGR DMYVDQELDI NRLSDYDVDH IVPQSFLKDD | |
| | SIDNKVLTRS DKNRGKSDNV PSEEVVKKMK NYWRQLLNAK LITQRKFDNL | |
| | TKAERGGLSE LDKAGFIKRQ LVETRQITKH VAQILDSRMN TKYDENDKLI | |
| | REVKVITLKS KLVSDFRKDF QFYKVREINN YHHAHDAYLN AVVGTALIKK | |
| | YPKLESEFVY GDYKVYDVRK MIAKSEQEIG KATAKYFFYS NIMNFFKTEI | |
| | TLANGEIRKR PLIETNGETG EIVWDKGRDF ATVRKVLSMP QVNIVKKTEV | |
| | QTGGFSKESI LPKRNSDKLI ARKKDWDPKK YGGFDSPTVA YSVLVVAKVE | |
| | KGKSKKLKSV KELLGITIME RSSFEKNPID FLEAKGYKEV KKDLIIKLPK | |
| | YSLFELENGR KRMLASAGEL QKGNELALPS KYVNFLYLAS HYEKLKGSPE | |
| | DNEQKQLFVE QHKHYLDEII EQISEFSKRV ILADANLDKV LSAYNKHRDK | |
| | PIREQAENII HLFTLTNLGA PAAFKYFDTT IDRKRYTSTK EVLDATLIHQ | |
| | SITGLYETRI DLS | |
| AJN60021.1 | MKRNYILGLD IGITSVGYGI IDYETRDVID AGVRLFKEAN VENNEGRRSK | (SEQ |
| GI: 757015977 | RGARRLKRRR RHRIQRVKKL LFDYNLLTDH SELSGINPYE ARVKGLSQKL | ID NO: |
| J7RUA5.1 | SEEEFSAALL HLAKRRGVHN VNEVEEDTGN ELSTKEQISR NSKALEEKYV | 22) |
| WP_05301979 | AELQLERLKK DGEVRGSINR FKTSDYVKEA KQLLKVQKAY HQLDQSFIDT | |
| 4.1 | YIDLLETRRT YYEGPGEGSP FGWKDIKEWY EMLMGHCTYF PEELRSVKYA | |
| Staphylococcus | YNADLYNALN DLNNLVITRD ENEKLEYYEK FQIIENVFKQ KKKPTLKQIA | |
| aureus | KEILVNEEDI KGYRVTSTGK PEFTNLKVYH DIKDITARKE IIENAELLDQ | |
| | IAKILTIYQS SEDIQEELTN LNSELTQEEI EQISNLKGYT GTHNLSLKAI | |
| | NLILDELWHT NDNQIAIFNR LKLVPKKVDL SQQKEIPTTL VDDFILSPVV | |
| | KRSFIQSIKV INAIIKKYGL PNDIIIELAR EKNSKDAQKM INEMQKRNRQ | |
| | TNERIEEIIR TTGKENAKYL IEKIKLHDMQ EGKCLYSLEA IPLEDLLNNP | |
| | FNYEVDHIIP RSVSFDNSFN NKVLVKQEEN SKKGNRTPFQ YLSSSDSKIS | |
| | YETFKKHILN LAKGKGRISK TKKEYLLEER DINRFSVQKD FINRNLVDTR | |
| | YATRGLMNLL RSYFRVNNLD VKVKSINGGF TSFLRRKWKF KKERNKGYKH | |
| | HAEDALIIAN ADFIFKEWKK LDKAKKVMEN QMFEEKQAES MPEIETEQEY | |
| | KEIFITPHQI KHIKDFKDYK YSHRVDKKPN RELINDTLYS TRKDDKGNTL | |
| | IVNNLNGLYD KDNDKLKKLI NKSPEKLLMY HHDPQTYQKL KLIMEQYGDE | |
| | KNPLYKYYEE TGNYLTKYSK KDNGPVIKKI KYYGNKLNAH LDITDDYPNS | |
| | RNKVVKLSLK PYRFDVYLDN GVYKFVTVKN LDVIKKENYY EVNSKCYEEA | |
| | KKLKKISNQA EFIASFYNND LIKINGELYR VIGVNNDLLN RIEVNMIDIT | |
| | YREYLENMND KRPPRIIKTI ASKTQSIKKY STDILGNLYE VKSKKHPQII | |
| | KKG | |
| AJN60008.1 | MARILAFDIG ISSIGWAFSE NDELKDCGVR IFTKVENPKT GESLALPRRL | (SEQ |
| GI: 757015964 | ARSARKRLAR RKARLNHLKH LIANEFKLNY EDYQSFDESL AKAYKGSLIS | ID NO: |
| WP_00286448 | PYELRFRALN ELLSKQDFAR VILHIAKRRG YDDIKNSDDK EKGAILKAIK | 23) |
| 5.1 | QNEEKLANYQ SVGEYLYKEY FQKFKENSKE FTNVRNKKES YERCIAQSFL | |
| Campylobacter | KDELKLIFKK QREFGFSFSK KFEEEVLSVA FYKRALKDFS HLVGNCSFFT | |
| jejuni subsp. | DEKRAPKNSP LAFMFVALTR IINLLNNLKN TEGILYTKDD LNALLNEVLK | |
| jejuni NCTC | NGTLTYKQTK KLLGLSDDYE FKGEKGTYFI EFKKYKEFIK ALGEHNLSQD | |
| 11168 = ATCC | DLNEIAKDIT LIKDEIKLKK ALAKYDLNQN QIDSLSKLEF KDHLNISFKA | |
| 700819 | LKLVTPLMLE GKKYDEACNE LNLKVAINED KKDFLPAFNE TYYKDEVTNP | |
| | VVLRAIKEYR KVLNALLKKY GKVHKINIEL AREVGKNHSQ RAKIEKEQNE | |
| | NYKAKKDAEL ECEKLGLKIN SKNILKLRLF KEQKEFCAYS GEKIKISDLQ | |
| | DEKMLEIDHI YPYSRSFDDS YMNKVLVFTK QNQEKLNQTP FEAFGNDSAK | |
| | WQKIEVLAKN LPTKKQKRIL DKNYKDKEQK NFKDRNLNDT RYIARLVLNY | |
| | TKDYLDFLPL SDDENTKLND TQKGSKVHVE AKSGMLTSAL RHTWGFSAKD | |
| | RNNHLHHAID AVIIAYANNS IVKAFSDFKK EQESNSAELY AKKISELDYK | |
| | NKRKFFEPFS GFRQKVLDKI DEIFVSKPER KKPSGALHEE TFRKEEEFYQ | |
| | SYGGKEGVLK ALELGKIRKV NGKIVKNGDM FRVDIFKHKK TNKFYAVPIY | |
| | TMDFALKVLP NKAVARSKKG EIKDWILMDE NYEFCFSLYK DSLILIQTKD | |
| | MQEPEFVYYN AFTSSTVSLI VSKHDNKFET LSKNQKILFK NANEKEVIAK | |
| | SIGIQNLKVF EKYIVSALGE VTKAEFRQRE DFKK | |
| Streptococcus | MSDLVLGLDI GIGSVGVGIL NKVTGEIIHK NSRIFPAAQA ENNLVRRTNR | (SEQ |
| thermophilus | QGRRLARRKK HRRVRLNRLF EESGLITDFT KISINLNPYQ LRVKGLTDEL | ID NO: |
| LMD-9 | SNEELFIALK NMVKHRGISY LDDASDDGNS SVGDYAQIVK ENSKQLETKT | 24) |
| AJN60026.1 | PGQIQLERYQ TYGQLRGDFT VEKDGKKHRL INVFPTSAYR SEALRILQTQ | |
| GI: 757015982 | QEFNPQITDE FINRYLEILT GKRKYYHGPG NEKSRTDYGR YRTSGETLDN | |
| WP_01168095 | IFGILIGKCT FYPDEFRAAK ASYTAQEFNL LNDLNNLTVP TETKKLSKEQ | |
| 7.1 | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARETNEDDEK KAIQKIQKAN KDEKDAAMLK AANQYNGKAE | |
| | LPHSVFHGHK QLATKIRLWH QQGERCLYTG KTISIHDLIN NSNQFEVDHI | |
| | LPLSITFDDS LANKVLVYAT ANQEKGQRTP YQALDSMDDA WSFRELKAFV | |
| | RESKTLSNKK KEYLLTEEDI SKFDVRKKFI ERNLVDTRYA SRVVLNALQE | |
| | HFRAHKIDTK VSWVRGQFTS QLRRHWGIEK TRDTYHHHAV DALIIAASSQ | |
| | LNLWKKQKNT LVSYSEDQLL DIETGELISD DEYKESVFKA PYQHFVDTLK | |
| | SKEFEDSILF SYQVDSKFNR KISDATIYAT RQAKVGKDKA DETYVLGKIK | |
| | DIYTQDGYDA FMKIYKKDKS KFLMYRHDPQ TFEKVIEPIL ENYPNKQINE | |
| | KGKEVPCNPF LKYKEEHGYI RKYSKKGNGP EIKSLKYYDS KLGNHIDITP | |
| | KDSNNKVVLQ SVSPWRADVY FNKTTGKYEI LGLKYADLQF EKGTGTYKIS | |
| | QEKYNDIKKK EGVDSDSEFK FTLYKNDLLL VKDTETKEQQ LFRFLSRTMP | |
| | KQKHYVELKP YDKQKFEGGE ALIKVLGNVA NSGQCKKGLG KSNISIYKVR | |
| | TDVLGNQHII KNEGDKPKLD F | |
| Parvibaculum | MERIFGFDIG TTSIGFSVID YSSTQSAGNI QRLGVRIFPE ARDPDGTPLN | (SEQ |
| lavamentivorans | QQRRQKRMMR RQLRRRRIRR KALNETLHEA GFLPAYGSAD WPVVMADEPY | ID NO: |
| DS-1 | ELRRRGLEEG LSAYEFGRAI YHLAQHRHFK GRELEESDTP DPDVDDEKEA | 25) |
| AJN60020.1 | ANERAATLKA LKNEQTTLGA WLARRPPSDR KRGIHAHRNV VAEEFERLWE | |
| GI: 757015976 | VQSKFHPALK SEEMRARISD TIFAQRPVFW RKNTLGECRF MPGEPLCPKG | |
| WP_01199501 | SWLSQQRRML EKLNNLAIAG GNARPLDAEE RDAILSKLQQ QASMSWPGVR | |
| 3.1 | SALKALYKQR GEPGAEKSLK FNLELGGESK LLGNALEAKL ADMFGPDWPA | |
| | HPRKQEIRHA VHERLWAADY GETPDKKRVI ILSEKDRKAH REAAANSFVA | |
| | DFGITGEQAA QLQALKLPTG WEPYSIPALN LFLAELEKGE RFGALVNGPD | |
| | WEGWRRTNFP HRNQPTGEIL DKLPSPASKE ERERISQLRN PTWVRTQNEL | |
| | RKWVNNLIGL YGKPDRIRIE VGRDVGKSKR EREEIQSGIR RNEKQRKKAT | |
| | EDLIKNGIAN PSRDDVEKWI LWKEGQERCP YTGDQIGFNA LFREGRYEVE | |
| | HIWPRSRSFD NSPRNKTLCR KDVNIEKGNR MPFEAFGHDE DRWSAIQIRL | |
| | QGMVSAKGGT GMSPGKVKRF LAKTMPEDFA ARQLNDTRYA AKQILAQLKR | |
| | LWPDMGPEAP VKVEAVTGQV TAQLRKLWTL NNILADDGEK TRADHRHHAI | |
| | DALTVACTHP GMTNKLSRYW QLRDDPRAEK PALTPPWDTI RADAEKAVSE | |
| | IVVSHRVRKK VSGPLHKETT YGDTGTDIKT KSGTYRQFVT RKKIESLSKG | |
| | ELDEIRDPRI KEIVAAHVAG RGGDPKKAFP PYPCVSPGGP EIRKVRLTSK | |
| | QQLNLMAQTG NGYADLGSNH HIAIYRLPDG KADFEIVSLF DASRRLAQRN | |
| | PIVQRTRADG ASFVMSLAAG EAIMIPEGSK KGIWIVQGVW ASGQVVLERD | |
| | TDADHSTTTR PMPNPILKDD AKKVSIDPIG RVRPSND | |
| Corynebacterium | MKYHVGIDVG TFSVGLAAIE VDDAGMPIKT LSLVSHIHDS GLDPDEIKSA | (SEQ |
| diphtheriae | VTRLASSGIA RRTRRLYRRK RRRLQQLDKF IQRQGWPVIE LEDYSDPLYP | ID NO: |
| NCTC 13129 | WKVRAELAAS YIADEKERGE KLSVALRHIA RHRGWRNPYA KVSSLYLPDG | 26) |
| AJN60012.1 | PSDAFKAIRE EIKRASGQPV PETATVGQMV TLCELGTLKL RGEGGVLSAR | |
| GI: 757015968 | LQQSDYAREI QEICRMQEIG QELYRKIIDV VFAAESPKGS ASSRVGKDPL | |
| WP_01093396 | QPGKNRALKA SDAFQRYRIA ALIGNLRVRV DGEKRILSVE EKNLVFDHLV | |
| 8.1 | NLTPKKEPEW VTIAEILGID RGQLIGTATM TDDGERAGAR PPTHDTNRSI | |
| | VNSRIAPLVD WWKTASALEQ HAMVKALSNA EVDDFDSPEG AKVQAFFADL | |
| | DDDVHAKLDS LHLPVGRAAY SEDTLVRLTR RMLSDGVDLY TARLQEFGIE | |
| | PSWTPPTPRI GEPVGNPAVD RVLKTVSRWL ESATKTWGAP ERVIIEHVRE | |
| | GFVTEKRARE MDGDMRRRAA RNAKLFQEMQ EKLNVQGKPS RADLWRYQSV | |
| | QRQNCQCAYC GSPITFSNSE MDHIVPRAGQ GSTNTRENLV AVCHRCNQSK | |
| | GNTPFAIWAK NTSIEGVSVK EAVERTRHWV TDTGMRSTDF KKFTKAVVER | |
| | FQRATMDEEI DARSMESVAW MANELRSRVA QHFASHGTTV RVYRGSLTAE | |
| | ARRASGISGK LKFFDGVGKS RLDRRHHAID AAVIAFTSDY VAETLAVRSN | |
| | LKQSQAHRQE APQWREFTGK DAEHRAAWRV WCQKMEKLSA LLTEDLRDDR | |
| | WVVMSNVRLR LGNGSAHKET IGKLSKVKLS SQLSVSDIDK ASSEALWCAL | |
| | TREPGFDPKE GLPANPERHI RVNGTHVYAG DNIGLFPVSA GSIALRGGYA | |
| | ELGSSFHHAR VYKITSGKKP AFAMLRVYTI DLLPYRNQDL FSVELKPQTM | |
| | SMRQAEKKLR DALATGNAEY LGWLVVDDEL VVDTSKIATD QVKAVEAELG | |
| | TIRRWRVDGF FSPSKLRLRP LQMSKEGIKK ESAPELSKII DRPGWLPAVN | |
| | KLFSDGNVTV VRRDSLGRVR LESTAHLPVT WKVQ | |
| Streptococcus | MTNGKILGLD IGIASVGVGI IEAKTGKVVH ANSRLFSAAN AENNAERRGF | (SEQ |
| pasteurianus | RGSRRLNRRK KHRVKRVRDL FEKYGIVTDF RNLNLNPYEL RVKGLTEQLK | ID NO: |
| WP_01385204 | NEELFAALRT ISKRRGISYL DDAEDDSTGS TDYAKSIDEN RRLLKNKTPG | 27) |
| 8.1 | QIQLERLEKY GQLRGNFTVY DENGEAHRLI NVFSTSDYEK EARKILETQA | |
| | DYNKKITAEF IDDYVEILTQ KRKYYHGPGN EKSRTDYGRF RTDGTTLENI | |
| | FGILIGKCNF YPDEYRASKA SYTAQEYNFL NDLNNLKVST ETGKLSTEQK | |
| | ESLVEFAKNT ATLGPAKLLK EIAKILDCKV DEIKGYREDD KGKPDLHTFE | |
| | PYRKLKFNLE SINIDDLSRE VIDKLADILT LNTEREGIED AIKRNLPNQF | |
| | TEEQISEIIK VRKSQSTAFN KGWHSFSAKL MNELIPELYA TSDEQMTILT | |
| | RLEKFKVNKK SSKNTKTIDE KEVTDEIYNP VVAKSVRQTI KIINAAVKKY | |
| | GDFDKIVIEM PRDKNADDEK KFIDKRNKEN KKEKDDALKR AAYLYNSSDK | |
| | LPDEVFHGNK QLETKIRLWY QQGERCLYSG KPISIQELVH NSNNFEIDHI | |
| | LPLSLSFDDS LANKVLVYAW TNQEKGQKTP YQVIDSMDAA WSFREMKDYV | |
| | LKQKGLGKKK RDYLLTTENI DKIEVKKKFI ERNLVDTRYA SRVVLNSLQS | |
| | ALRELGKDTK VSVVRGQFTS QLRRKWKIDK SRETYHHHAV DALIIAASSQ | |
| | LKLWEKQDNP MFVDYGKNQV VDKQTGEILS VSDDEYKELV FQPPYQGFVN | |
| | TISSKGFEDE ILFSYQVDSK YNRKVSDATI YSTRKAKIGK DKKEETYVLG | |
| | KIKDIYSQNG FDTFIKKYNK DKTQFLMYQK DSLTWENVIE VILRDYPTTK | |
| | KSEDGKNDVK CNPFEEYRRE NGLICKYSKK GKGTPIKSLK YYDKKLGNCI | |
| | DITPEESRNK VILQSINPWR ADVYFNPETL KYELMGLKYS DLSFEKGTGN | |
| | YHISQEKYDA IKEKEGIGKK SEFKFTLYRN DLILIKDIAS GEQEIYRFLS | |
| | RTMPNVNHYV ELKPYDKEKF DNVQELVEAL GEADKVGRCI KGLNKPNISI | |
| | YKVRTDVLGN KYFVKKKGDK PKLDFKNNK K | |
| Neisseria | MAAFKPNPMN YILGLDIGIA SVGWAIVEID EEENPIRLID LGVRVFERAE | (SEQ |
| cinerea ATCC | VPKTGDSLAA ARRLARSVRR LTRRRAHRLL RARRLLKREG VLQAADFDEN | ID NO: |
| 14685 | GLIKSLPNTP WQLRAAALDR KLTPLEWSAV LLHLIKHRGY LSQRKNEGET | 28) |
| AJN60019.1 | ADKELGALLK GVADNTHALQ TGDFRTPAEL ALNKFEKESG HIRNQRGDYS | |
| GI: 757015975 | HTFNRKDLQA ELNLLFEKQK EFGNPHVSDG LKEGIETLLM TQRPALSGDA | |
| WP_00367641 | VQKMLGHCTF EPTEPKAAKN TYTAERFVWL TKLNNLRILE QGSERPLTDT | |
| 0.1 | ERATLMDEPY RKSKLTYAQA RKLLDLDDTA FFKGLRYGKD NAEASTLMEM | |
| | KAYHAISRAL EKEGLKDKKS PLNLSPELQD EIGTAFSLFK TDEDITGRLK | |
| | DRVQPEILEA LLKHISFDKF VQISLKALRR IVPLMEQGNR YDEACTEIYG | |
| | DHYGKKNTEE KIYLPPIPAD EIRNPVVLRA LSQARKVING VVRRYGSPAR | |
| | IHIETAREVG KSFKDRKEIE KRQEENRKDR EKSAAKFREY FPNFVGEPKS | |
| | KDILKLRLYE QQHGKCLYSG KEINLGRLNE KGYVEIDHAL PFSRTWDDSF | |
| | NNKVLALGSE NQNKGNQTPY EYFNGKDNSR EWQEFKARVE TSRFPRSKKQ | |
| | RILLQKFDED GFKERNLNDT RYINRFLCQF VADHMLLTGK GKRRVFASNG | |
| | QITNLLRGFW GLRKVRAEND RHHALDAVVV ACSTIAMQQK ITRFVRYKEM | |
| | NAFDGKTIDK ETGEVLHQKA HFPQPWEFFA QEVMIRVFGK PDGKPEFEEA | |
| | DTPEKLRTLL AEKLSSRPEA VHKYVTPLFI SRAPNRKMSG QGHMETVKSA | |
| | KRLDEGISVL RVPLTQLKLK DLEKMVNRER EPKLYEALKA RLEAHKDDPA | |
| | KAFAEPFYKY DKAGNRTQQV KAVRVEQVQK TGVWVHNHNG IADNATIVRV | |
| | DVFEKGGKYY LVPIYSWQVA KGILPDRAWV QGKDEEDWTV MDDSFEFKFV | |
| | LYANDLIKLT AKKNEFLGYF VSLNRATGAI DIRTHDTDST KGKNGIFQSV | |
| | GVKTALSFQK YQIDELGKEI RPCRLKKRPP VR | |
| AJN60009.1 | MSDLVLGLDI GIGSVGVGIL NKVTGEIIHK NSRIFPAAQA ENNLVRRTNR | (SEQ |
| GI: 757015965 | QGRRLARRKK HRRVRLNRLF EESGLITDFT KISINLNPYQ LRVKGLTDEL | ID NO: |
| St1Cas9 + | SNEELFIALK NMVKHRGISY LDDASDDGNS SVGDYAQIVK ENSKQLETKT | 29) |
| SpCas9 | PGQIQLERYQ TYGQLRGDFT VEKDGKKHRL INVFPTSAYR SEALRILQTQ | |
| | QEFNPQITDE FINRYLEILT GKRKYYHGPG NEKSRTDYGR YRTSGETLDN | |
| | IFGILIGKCT FYPDEFRAAK ASYTAQEFNL LNDLNNLTVP TETKKLSKEQ | |
| | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARENQTTQKG QKNSRERMKR IEEGIKELGS QILKEHPVEN | |
| | TQLQNEKLYL YYLQNGRDMY VDQELDINRL SDYDVDHIVP QSFLKDDSID | |
| | NKVLTRSDKN RGKSDNVPSE EVVKKMKNYW RQLLNAKLIT QRKFDNLTKA | |
| | ERGGLSELDK AGFIKRQLVE TRQITKHVAQ ILDSRMNTKY DENDKLIREV | |
| | KVITLKSKLV SDFRKDFQFY KVREINNYHH AHDAYLNAVV GTALIKKYPK | |
| | LESEFVYGDY KVYDVRKMIA KSEQEIGKAT AKYFFYSNIM NFFKTEITLA | |
| | NGEIRKRPLI ETNGETGEIV WDKGRDFATV RKVLSMPQVN IVKKTEVQTG | |
| | GFSKESILPK RNSDKLIARK KDWDPKKYGG FDSPTVAYSV LWVAKVEKGK | |
| | SKKLKSVKEL LGITIMERSS FEKNPIDFLE AKGYKEVKKD LIIKLPKYSL | |
| | FELENGRKRM LASAGELQKG NELALPSKYV NFLYLASHYE KLKGSPEDNE | |
| | QKQLFVEQHK HYLDEIIEQI SEFSKRVILA DANLDKVLSA YNKHRDKPIR | |
| | EQAENIIHLF TLTNLGAPAA FKYFDTTIDR KRYTSTKEVL DATLIHQSIT | |
| | GLYETRIDLS QLGGD | |
| Campylobacter | MRILGFDIGI NSIGWAFVEN DELKDCGVRI FTKAENPKNK ESLALPRRNA | (SEQ |
| lari Cas9 | RSSRRRLKRR KARLIAIKRI LAKELKLNYK DYVAADGELP KAYEGSLASV | ID NO: |
| BAK69486.1 | YELRYKALTQ NLETKDLARV ILHIAKHRGY MNKNEKKSND AKKGKILSAL | 30) |
| | KNNALKLENY QSVGEYFYKE FFQKYKKNTK NFIKIRNTKD NYNNCVLSSD | |
| | LEKELKLILE KQKEFGYNYS EDFINEILKV AFFQRPLKDF SHLVGACTFF | |
| | EEEKRACKNS YSAWEFVALT KIINEIKSLE KISGEIVPTQ TINEVLNLIL | |
| | DKGSITYKKF RSCINLHESI SFKSLKYDKE NAENAKLIDF RKLVEFKKAL | |
| | GVHSLSRQEL DQISTHITLI KDNVKLKTVL EKYNLSNEQI NNLLEIEFND | |
| | YINLSFKALG MILPLMREGK RYDEACEIAN LKPKTVDEKK DFLPAFCDSI | |
| | FAHELSNPVV NRAISEYRKV LNALLKKYGK VHKIHLELAR DVGLSKKARE | |
| | KIEKEQKENQ AVNAWALKEC ENIGLKASAK NILKLKLWKE QKEICIYSGN | |
| | KISIEHLKDE KALEVDHIYP YSRSFDDSFI NKVLVFTKEN QEKLNKTPFE | |
| | AFGKNIEKWS KIQTLAQNLP YKKKNKILDE NFKDKQQEDF ISRNLNDTRY | |
| | IATLIAKYTK EYLNFLLLSE NENANLKSGE KGSKIHVQTI SGMLTSVLRH | |
| | TWGFDKKDRN NHLHHALDAI IVAYSTNSII KAFSDFRKNQ ELLKARFYAK | |
| | ELTSDNYKHQ VKFFEPFKSF REKILSKIDE IFVSKPPRKR ARRALHKDTF | |
| | HSENKIIDKC SYNSKEGLQI ALSCGRVRKI GTKYVENDTI VRVDIFKKQN | |
| | KFYAIPIYAM DFALGILPNK IVITGKDKNN NPKQWQTIDE SYEFCFSLYK | |
| | NDLILLQKKN MQEPEFAYYN DFSISTSSIC VEKHDNKFEN LTSNQKLLFS | |
| | NAKEGSVKVE SLGIQNLKVF EKYIITPLGD KIKADFQPRE NISLKTSKKY | |
| | GLR | |
| AJN60010.1 | MDKKYSIGLD IGTNSVGWAV ITDEYKVPSK KFKVLGNTDR HSIKKNLIGA | (SEQ |
| GI: 757015966 | LLFDSGETAE ATRLKRTARR RYTRRKNRIC YLQEIFSNEM AKVDDSFFHR | ID NO: |
| SpCas9 + | LEESFLVEED KKHERHPIFG NIVDEVAYHE KYPTIYHLRK KLVDSTDKAD | 31) |
| St1Cas9 | LRLIYLALAH MIKFRGHFLI EGDLNPDNSD VDKLFIQLVQ TYNQLFEENP | |
| | INASGVDAKA ILSARLSKSR RLENLIAQLP GEKKNGLFGN LIALSLGLTP | |
| | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNFDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRFNAS LGTYHDLLKI | |
| | IKDKDFLDNE ENEDILEDIV LTLTLFEDRE MIEERLKTYA HLFDDKVMKQ | |
| | LKRRRYTGWG RLSRKLINGI RDKQSGKTIL DFLKSDGFAN RNFMQLIHDD | |
| | SLTFKEDIQK AQVSGQGDSL HEHIANLAGS PAIKKGILQT VKVVDELVKV | |
| | MGRHKPENIV IEMARETNED DEKKAIQKIQ KANKDEKDAA MLKAANQYNG | |
| | KAELPHSVFH GHKQLATKIR LWHQQGERCL YTGKTISIHD LINNSNQFEV | |
| | DHILPLSITF DDSLANKVLV YATANQEKGQ RTPYQALDSM DDAWSFRELK | |
| | AFVRESKTLS NKKKEYLLTE EDISKFDVRK KFIERNLVDT RYASRVVLNA | |
| | LQEHFRAHKI DTKVSVVRGQ FTSQLRRHWG IEKTRDTYHH HAVDALIIAA | |
| | SSQLNLWKKQ KNTLVSYSED QLLDIETGEL ISDDEYKESV FKAPYQHFVD | |
| | TLKSKEFEDS ILFSYQVDSK FNRKISDATI YATRQAKVGK DKADETYVLG | |
| | KIKDIYTQDG YDAFMKIYKK DKSKFLMYRH DPQTFEKVIE PILENYPNKQ | |
| | INEKGKEVPC NPFLKYKEEH GYIRKYSKKG NGPEIKSLKY YDSKLGNHID | |
| | ITPKDSNNKV VLQSVSPWRA DVYFNKTTGK YEILGLKYAD LQFEKGTGTY | |
| | KISQEKYNDI KKKEGVDSDS EFKFTLYKND LLLVKDTETK EQQLFRFLSR | |
| | TMPKQKHYVE LKPYDKQKFE GGEALIKVLG NVANSGQCKK GLGKSNISIY | |
| | KVRTDVLGNQ HIIKNEGDKP KLDF | |
| SpCas9 | MDKKYSIGLD IGTNSVGWAV ITDEYKVPSK KFKVLGNTDR HSIKKNLIGA | (SEQ |
| inactive | LLFDSGETAE ATRLKRTARR RYTRRKNRIC YLQEIFSNEM AKVDDSFFHR | ID NO: |
| AJN60011.1 | LEESFLVEED KKHERHPIFG NIVDEVAYHE KYPTIYHLRK KLVDSTDKAD | 32) |
| GI: 757015967 | LRLIYLALAH MIKFRGHFLI EGDLNPDNSD VDKLFIQLVQ TYNQLFEENP | |
| | INASGVDAKA ILSARLSKSR RLENLIAQLP GEKKNGLFGN LIALSLGLTP | |
| | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNFDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRFNAS LGTYHDLLKI | |
| | IKDKDFLDNE ENEDILEDIV LTLTLFEDRE MIEERLKTYA HLFDDKVMKQ | |
| | LKRRRYTGWG RLSRKLINGI RDKQSGKTIL DFLKSDGFAN RNFMQLIHDD | |
| | SLTFKEDIQK AQVSGQGDSL HEHIANLAGS PAIKKGILQT VKVVDELVKV | |
| | MGRHKPENIV IAMARENQTT QKGQKNSRER MKRIEEGIKE LGSQILKEHP | |
| | VENTQLQNEK LYLYYLQNGR DMYVDQELDI NRLSDYDVDA IVPQSFLKDD | |
| | SIDAKVLTRS DKARGKSDNV PSEEVVKKMK NYWRQLLNAK LITQRKFDNL | |
| | TKAERGGLSE LDKAGFIKRQ LVETRQITKH VAQILDSRMN TKYDENDKLI | |
| | REVKVITLKS KLVSDFRKDF QFYKVREINN YHHAHAAYLN AVVGTALIKK | |
| | YPKLESEFVY GDYKVYDVRK MIAKSEQEIG KATAKYFFYS NIMNFFKTEI | |
| | TLANGEIRKR PLIETNGETG EIVWDKGRDF ATVRKVLSMP QVNIVKKTEV | |
| | QTGGFSKESI LPKRNSDKLI ARKKDWDPKK YGGFDSPTVA YSVLVVAKVE | |
| | KGKSKKLKSV KELLGITIME RSSFEKNPID FLEAKGYKEV KKDLIIKLPK | |
| | YSLFELENGR KRMLASAGEL QKGNELALPS KYVNFLYLAS HYEKLKGSPE | |
| | DNEQKQLFVE QHKHYLDEII EQISEFSKRV ILADANLDKV LSAYNKHRDK | |
| | PIREQAENII HLFTLTNLGA PAAFKYFDTT IDRKRYTSTK EVLDATLIHQ | |
| | SITGLYETRI DLSQLGGD | |
| AJN60013.1 | MTQSERRFSC SIGIDMGAKY TGVFYALFDR EELPTNLNSK AMTLVMPETG | (SEQ |
| GI: 757015969 | PRYVQAQRTA VRHRLRGQKR YTLARKLAFL VVDDMIKKQE KRLTDEEWKR | ID NO: |
| WP_ | GREALSGLLK RRGYSRPNAD GEDLTPLENV RADVFAAHPA FSTYFSEVRS | 33) |
| 005430658.1 | LAEQWEEFTA NISNVEKFLG DPNIPADKEF IEFAVAEGLI DKTEKKAYQS | |
| Sutterella | ALSTLRANAN VLTGLRQMGH KPRSEYFKAI EADLKKDSRL AKINEAFGGA | |
| wadsworthensis | ERLARLLGNL SNLQLRAERW YFNAPDIMKD RGWEPDRFKK TLVRAFKFFH | |
| 3_1_45B | PAKDQNKQHL ELIKQIENSE DIIETLCTLD PNRTIPPYED QNNRRPPLDQ | |
| | TLLLSPEKLT RQYGEIWKTW SARLTSAEPT LAPAAEILER STDRKSRVAV | |
| | NGHEPLPTLA YQLSYALQRA FDRSKALDPY ALRALAAGSK SNKLTSARTA | |
| | LENCIGGQNV KTFLDCARRY YREADDAKVG LWFDNADGLL ERSDLHPPMK | |
| | KKILPLLVAN ILQTDETTGQ KFLDEIWRKQ IKGRETVASR CARIETVRKS | |
| | FGGGFNIAYN TAQYREVNKL PRNAQDKELL TIRDRVAETA DFIAANLGLS | |
| | DEQKRKFANP FSLAQFYTLI ETEVSGFSAT TLAVHLENAW RMTIKDAVIN | |
| | GETVRAAQCS RLPAETARPF DGLVRRLVDR QAWEIAKRVS TDIQSKVDFS | |
| | NGIVDVSIFV EENKFEFSAS VADLKKNKRV KDKMLSEAEK LETRWLIKNE | |
| | RIKKASRGTC PYTGDRLAEG GEIDHILPRS LIKDARGIVE NAEPNLIYAS | |
| | SRGNQLKKNQ RYSLSDLKAN YRNEIFKTSN IAAITAEIED VVTKLQQTHR | |
| | LKFFDLLNEH EQDCVRHALF LDDGSEARDA VLELLATQRR TRVNGTQIWM | |
| | IKNLANKIRE ELQNWCKTTN NRLHFQAAAT NVSDAKNLRL KLAQNQPDFE | |
| | KPDIQPIASH SIDALCSFAV GSADAERDQN GFDYLDGKTV LGLYPQSCEV | |
| | IHLQAKPQEE KSHFDSVAIF KEGIYAEQFL PIFTLNEKIW IGYETLNAKG | |
| | ERCGAIEVSG KQPKELLEML APFFNKPVGD LSAHATYRIL KKPAYEFLAK | |
| | AALQPLSAEE KRLAALLDAL RYCTSRKSLM SLFMAANGKS LKKREDVLKP | |
| | KLFQLKVELK GEKSFKLNGS LTLPVKQDWL RICDSPELAD AFGKPCSADE | |
| | LTSKLARIWK RPVMRDLAHA PVRREFSLPA IDNPSGGFRI RRTNLFGNEL | |
| | YQVHAINAKK YRGFASAGSN VDWSKGILFN ELQHENLTEC GGRFITSADV | |
| | TPMSEWRKVV AEDNLSIWIA PGTEGRRYVR VETTFIQASH WFEQSVENWA | |
| | ITSPLSLPAS FKVDKPAEFQ KAVGTELSEL LGQPRSEIFI ENVGNAKHIR | |
| | FWYIVVSSNK KMNESYNNVS KS | |
| AJN60014.1 | MESSQILSPI GIDLGGKFTG VCLSHLEAFA ELPNHANTKY SVILIDHNNF | (SEQ |
| GI: 757015970 | QLSQAQRRAT RHRVRNKKRN QFVKRVALQL FQHILSRDLN AKEETALCHY | ID NO: |
| WP_01121279 | LNNRGYTYVD TDLDEYIKDE TTINLLKELL PSESEHNFID WFLQKMQSSE | 34) |
| 2.1 | FRKILVSKVE EKKDDKELKN AVKNIKNFIT GFEKNSVEGH RHRKVYFENI | |
| Legionella | KSDITKDNQL DSIKKKIPSV CLSNLLGHLS NLQWKNLHRY LAKNPKQFDE | |
| pneumophila | QTFGNEFLRM LKNFRHLKGS QESLAVRNLI QQLEQSQDYI SILEKTPPEI | |
| str. Paris | TIPPYEARTN TGMEKDQSLL LNPEKLNNLY PNWRNLIPGI IDAHPFLEKD | |
| | LEHTKLRDRK RIISPSKQDE KRDSYILQRY LDLNKKIDKF KIKKQLSFLG | |
| | QGKQLPANLI ETQKEMETHF NSSLVSVLIQ IASAYNKERE DAAQGIWFDN | |
| | AFSLCELSNI NPPRKQKILP LLVGAILSED FINNKDKWAK FKIFWNTHKI | |
| | GRTSLKSKCK EIEEARKNSG NAFKIDYEEA LNHPEHSNNK ALIKIIQTIP | |
| | DIIQAIQSHL GHNDSQALIY HNPFSLSQLY TILETKRDGF HKNCVAVTCE | |
| | NYWRSQKTEI DPEISYASRL PADSVRPFDG VLARMMQRLA YEIAMAKWEQ | |
| | IKHIPDNSSL LIPIYLEQNR FEFEESFKKI KGSSSDKTLE QAIEKQNIQW | |
| | EEKFQRIINA SMNICPYKGA SIGGQGEIDH IYPRSLSKKH FGVIFNSEVN | |
| | LIYCSSQGNR EKKEEHYLLE HLSPLYLKHQ FGTDNVSDIK NFISQNVANI | |
| | KKYISFHLLT PEQQKAARHA LFLDYDDEAF KTITKFLMSQ QKARVNGTQK | |
| | FLGKQIMEFL STLADSKQLQ LEFSIKQITA EEVHDHRELL SKQEPKLVKS | |
| | RQQSFPSHAI DATLTMSIGL KEFPQFSQEL DNSWFINHLM PDEVHLNPVR | |
| | SKEKYNKPNI SSTPLFKDSL YAERFIPVWV KGETFAIGFS EKDLFEIKPS | |
| | NKEKLFTLLK TYSTKNPGES LQELQAKSKA KWLYFPINKT LALEFLHHYF | |
| | HKEIVTPDDT TVCHFINSLR YYTKKESITV KILKEPMPVL SVKFESSKKN | |
| | VLGSFKHTIA LPATKDWERL FNHPNFLALK ANPAPNPKEF NEFIRKYFLS | |
| | DNNPNSDIPN NGHNIKPQKH KAVRKVFSLP VIPGNAGTMM RIRRKDNKGQ | |
| | PLYQLQTIDD TPSMGIQINE DRLVKQEVLM DAYKTRNLST IDGINNSEGQ | |
| | AYATFDNWLT LPVSTFKPEI IKLEMKPHSK TRRYIRITQS LADFIKTIDE | |
| | ALMIKPSDSI DDPLNMPNEI VCKNKLFGNE LKPRDGKMKI VSTGKIVTYE | |
| | FESDSTPQWI QTLYVTQLKK QP | |
| AJN60015.1 | MKKEIKDYFL GLDVGTGSVG WAVTDTDYKL LKANRKDLWG MRCFETAETA | (SEQ |
| GI: 757015971 | EVRRLHRGAR RRIERRKKRI KLLQELFSQE IAKTDEGFFQ RMKESPFYAE | ID NO: |
| WP_00268128 | DKTILQENTL FNDKDFADKT YHKAYPTINH LIKAWIENKV KPDPRLLYLA | 35) |
| 9.1 | CHNIIKKRGH FLFEGDFDSE NQFDTSIQAL FEYLREDMEV DIDADSQKVK | |
| Treponema | EILKDSSLKN SEKQSRLNKI LGLKPSDKQK KAITNLISGN KINFADLYDN | |
| denticola | PDLKDAEKNS ISFSKDDFDA LSDDLASILG DSFELLLKAK AVYNCSVLSK | |
| ATCC 35405 | VIGDEQYLSF AKVKIYEKHK TDLTKLKNVI KKHFPKDYKK VFGYNKNEKN | |
| | NNNYSGYVGV CKTKSKKLII NNSVNQEDFY KFLKTILSAK SEIKEVNDIL | |
| | TEIETGTFLP KQISKSNAEI PYQLRKMELE KILSNAEKHF SFLKQKDEKG | |
| | LSHSEKIIML LTFKIPYYIG PINDNHKKFF PDRCWVVKKE KSPSGKTTPW | |
| | NFFDHIDKEK TAEAFITSRT NFCTYLVGES VLPKSSLLYS EYTVLNEINN | |
| | LQIIIDGKNI CDIKLKQKIY EDLFKKYKKI TQKQISTFIK HEGICNKTDE | |
| | VIILGIDKEC TSSLKSYIEL KNIFGKQVDE ISTKNMLEEI IRWATIYDEG | |
| | EGKTILKTKI KAEYGKYCSD EQIKKILNLK FSGWGRLSRK FLETVTSEMP | |
| | GFSEPVNIIT AMRETQNNLM ELLSSEFTFT ENIKKINSGF EDAEKQFSYD | |
| | GLVKPLFLSP SVKKMLWQTL KLVKEISHIT QAPPKKIFIE MAKGAELEPA | |
| | RTKTRLKILQ DLYNNCKNDA DAFSSEIKDL SGKIENEDNL RLRSDKLYLY | |
| | YTQLGKCMYC GKPIEIGHVF DTSNYDIDHI YPQSKIKDDS ISNRVLVCSS | |
| | CNKNKEDKYP LKSEIQSKQR GFWNFLQRNN FISLEKLNRL TRATPISDDE | |
| | TAKFIARQLV ETRQATKVAA KVLEKMFPET KIVYSKAETV SMFRNKFDIV | |
| | KCREINDFHH AHDAYLNIVV GNVYNTKFTN NPWNFIKEKR DNPKIADTYN | |
| | YYKVFDYDVK RNNITAWEKG KTIITVKDML KRNTPIYTRQ AACKKGELFN | |
| | QTIMKKGLGQ HPLKKEGPFS NISKYGGYNK VSAAYYTLIE YEEKGNKIRS | |
| | LETIPLYLVK DIQKDQDVLK SYLTDLLGKK EFKILVPKIK INSLLKINGF | |
| | PCHITGKTND SFLLRPAVQF CCSNNEVLYF KKIIRFSEIR SQREKIGKTI | |
| | SPYEDLSFRS YIKENLWKKT KNDEIGEKEF YDLLQKKNLE IYDMLLTKHK | |
| | DTIYKKRPNS ATIDILVKGK EKFKSLIIEN QFEVILEILK LFSATRNVSD | |
| | LQHIGGSKYS GVAKIGNKIS SLDNCILIYQ SITGIFEKRI DLLKV | |
| AJN60016.1 | MTKEYYLGLD VGTNSVGWAV TDSQYNLCKF KKKDMWGIRL FESANTAKDR | (SEQ |
| GI: 757015972 | RLQRGNRRRL ERKKQRIDLL QEIFSPEICK IDPTFFIRLN ESRLHLEDKS | ID NO: |
| EFE28295.1 | NDFKYPLFIE KDYSDIEYYK EFPTIFHLRK HLIESEEKQD IRLIYLALHN | 36) |
| Filifactor alocis | IIKTRGHFLI DGDLQSAKQL RPILDTFLLS LQEEQNLSVS LSENQKDEYE | |
| ATCC 35896 | EILKNRSIAK SEKVKKLKNL FEISDELEKE EKKAQSAVIE NFCKFIVGNK | |
| | GDVCKFLRVS KEELEIDSFS FSEGKYEDDI VKNLEEKVPE KVYLFEQMKA | |
| | MYDWNILVDI LETEEYISFA KVKQYEKHKT NLRLLRDIIL KYCTKDEYNR | |
| | MFNDEKEAGS YTAYVGKLKK NNKKYWIEKK RNPEEFYKSL GKLLDKIEPL | |
| | KEDLEVLTMM IEECKNHTLL PIQKNKDNGV IPHQVHEVEL KKILENAKKY | |
| | YSFLTETDKD GYSVVQKIES IFRFRIPYYV GPLSTRHQEK GSNVWMVRKP | |
| | GREDRIYPWN MEEIIDFEKS NENFITRMTN KCTYLIGEDV LPKHSLLYSK | |
| | YMVLNELNNV KVRGKKLPTS LKQKVFEDLF ENKSKVTGKN LLEYLQIQDK | |
| | DIQIDDLSGF DKDFKTSLKS YLDFKKQIFG EEIEKESIQN MIEDIIKWIT | |
| | IYGNDKEMLK RVIRANYSNQ LTEEQMKKIT GFQYSGWGNF SKMFLKGISG | |
| | SDVSTGETFD IITAMWETDN NLMQILSKKF TFMDNVEDFN SGKVGKIDKI | |
| | TYDSTVKEMF LSPENKRAVW QTIQVAEEIK KVMGCEPKKI FIEMARGGEK | |
| | VKKRTKSRKA QLLELYAACE EDCRELIKEI EDRDERDFNS MKLFLYYTQF | |
| | GKCMYSGDDI DINELIRGNS KWDRDHIYPQ SKIKDDSIDN LVLVNKTYNA | |
| | KKSNELLSED IQKKMHSFWL SLLNKKLITK SKYDRLTRKG DFTDEELSGF | |
| | IARQLVETRQ STKAIADIFK QIYSSEVVYV KSSLVSDFRK KPLNYLKSRR | |
| | VNDYHHAKDA YLNIVVGNVY NKKFTSNPIQ WMKKNRDTNY SLNKVFEHDV | |
| | VINGEVIWEK CTYHEDTNTY DGGTLDRIRK IVERDNILYT EYAYCEKGEL | |
| | FNATIQNKNG NSTVSLKKGL DVKKYGGYFS ANTSYFSLIE FEDKKGDRAR | |
| | HIIGVPIYIA NMLEHSPSAF LEYCEQKGYQ NVRILVEKIK KNSLLIINGY | |
| | PLRIRGENEV DTSFKRAIQL KLDQKNYELV RNIEKFLEKY VEKKGNYPID | |
| | ENRDHITHEK MNQLYEVLLS KMKKFNKKGM ADPSDRIEKS KPKFIKLEDL | |
| | IDKINVINKM LNLLRCDNDT KADLSLIELP KNAGSFVVKK NTIGKSKIIL | |
| | VNQSVTGLYE NRREL | |
| AJN60017.1 | MGRKPYILSL DIGTGSVGYA CMDKGFNVLK YHDKDALGVY LFDGALTAQE | (SEQ |
| GI: 757015973 | RRQFRTSRRR KNRRIKRLGL LQELLAPLVQ NPNFYQFQRQ FAWKNDNMDF | ID NO: |
| WP_01461325 | KNKSLSEVLS FLGYESKKYP TIYHLQEALL LKDEKFDPEL IYMALYHLVK | 37) |
| 9.1 | YRGHFLFDHL KIENLTNNDN MHDFVELIET YENLNNIKLN LDYEKTKVIY | |
| Staphylococcus | EILKDNEMTK NDRAKRVKNM EKKLEQFSIM LLGLKFNEGK LFNHADNAEE | |
| pseudintermedius | LKGANQSHTF ADNYEENLTP FLTVEQSEFI ERANKIYLSL TLQDILKGKK | |
| ED99 | SMAMSKVAAY DKFRNELKQV KDIVYKADST RTQFKKIFVS SKKSLKQYDA | |
| | TPNDQTFSSL CLFDQYLIRP KKQYSLLIKE LKKIIPQDSE LYFEAENDTL | |
| | LKVLNTTDNA SIPMQINLYE AETILRNQQK YHAEITDEMI EKVLSLIQFR | |
| | IPYYVGPLVN DHTASKFGWM ERKSNESIKP WNFDEVVDRS KSATQFIRRM | |
| | TNKCSYLINE DVLPKNSLLY QEMEVLNELN ATQIRLQTDP KNRKYRMMPQ | |
| | IKLFAVEHIF KKYKTVSHSK FLEIMLNSNH RENFMNHGEK LSIFGTQDDK | |
| | KFASKLSSYQ DMTKIFGDIE GKRAQIEEII QWITIFEDKK ILVQKLKECY | |
| | PELTSKQINQ LKKLNYSGWG RLSEKLLTHA YQGHSIIELL RHSDENFMEI | |
| | LTNDVYGFQN FIKEENQVQS NKIQHQDIAN LTTSPALKKG IWSTIKLVRE | |
| | LTSIFGEPEK IIMEFATEDQ QKGKKQKSRK QLWDDNIKKN KLKSVDEYKY | |
| | IIDVANKLNN EQLQQEKLWL YLSQNGKCMY SGQSIDLDAL LSPNATKHYE | |
| | VDHIFPRSFI KDDSIDNKVL VIKKMNQTKG DQVPLQFIQQ PYERIAYWKS | |
| | LNKAGLISDS KLHKLMKPEF TAMDKEGFIQ RQLVETRQIS VHVRDFLKEE | |
| | YPNTKVIPMK AKMVSEFRKK FDIPKIRQMN DAHHAIDAYL NGVVYHGAQL | |
| | AYPNVDLFDF NFKWEKVREK WKALGEFNTK QKSRELFFFK KLEKMEVSQG | |
| | ERLISKIKLD MNHFKINYSR KLANIPQQFY NQTAVSPKTA ELKYESNKSN | |
| | EVVYKGLTPY QTYVVAIKSV NKKGKEKMEY QMIDHYVFDF YKFQNGNEKE | |
| | LALYLAQREN KDEVLDAQIV YSLNKGDLLY INNHPCYFVS RKEVINAKQF | |
| | ELTVEQQLSL YNVMNNKETN VEKLLIEYDF IAEKVINEYH HYLNSKLKEK | |
| | RVRTFFSESN QTHEDFIKAL DELFKVVTAS ATRSDKIGSR KNSMTHRAFL | |
| | GKGKDVKIAY TSISGLKTTK PKSLFKLAES RNEL | |
| AJN60018.1 | MTKIKDDYIV GLDIGTDSCG WVAMNSNNDI LKLQGKTAIG SRLFEGGKSA | (SEQ |
| GI: 757015974 | AERRLFRTTH RRIKRRRWRL KLLEEFFDPY MAEVDPYFFA RLKESGLSPL | ID NO: |
| WP_01456756 | DKRKTVSSIV FPTSAEDKKF YDDYPTIYHL RYKLMTEDEK FDLREVYLAI | 38) |
| 1.1 | HHIIKYRGNF LYNTSVKDFK ASKIDVKSSI EKLNELYENL GLDLNVEFNI | |
| Lactobacillus | SNTAEIEKVL KDKQIFKRDK VKKIAELFAI KTDNKEQSKR IKDISKQVAN | |
| johnsonii DPC | AVLGYKTRFD TIALKEISKD ELSDWNFKLS DIDADSKFEA LMGNLDENEQ | |
| 6026 | AILLTIKELF NEVTLNGIVE DGNTLSESMI NKYNDHRDDL KLLKEVIENH | |
| | IDRKKAKELA LAYDLYVNNR HGQLLQAKKK LGKIKPRSKE DFYKVVNKNL | |
| | DDSRASKEIK KKIELDSFMP KQRTNANGVI PYQLQQLELD KIIENQSKYY | |
| | PFLKEINPVS SHLKEAPYKL DELIRFRVPY YVGPLISPNE STKDIQTKKN | |
| | QNFAWMIRKE EGRITPWNFD QKVDRIESAN KFIKRMTTKD TYLFGEDVLP | |
| | ANSLLYQKFT VLNELNNIRI NGKRISVDLK QEIYENLFKK HTTVTVKKLE | |
| | NYLKENHNLV KVEIKGLADE KKENSGLTTY NRFKNLNIFD NQIDDLKYRN | |
| | DFEKIIEWST IFEDKSIYKE KLRSIDWLNE KQINALSNIR LQGWGRLSKK | |
| | LLAQLHDHNG QTIIEQLWDS QNNFMQIVTQ ADFKDAIAKA NQNLLVATSV | |
| | EDILNNAYTS PANKKAIRQV IKVVDDIVKA ASGKVPKQIA IEFTRDADEN | |
| | PKRSQTRGSK LQKVYKDLST ELASKTIAEE LNEAIKDKKL VQDKYYLYFM | |
| | QLGRDAYTGE PINIDEIQKY DIDHILPQSF IKDDALDNRV LVSRAVNNGK | |
| | SDNVPVKLFG NEMAANLGMT IRKMWEEWKN IGLISKTKYN NLLTDPDHIN | |
| | KYKSAGFIRR QLVETSQIIK LVSTILQSRY PNTEIITVKA KYNHYLREKF | |
| | DLYKSREVND YHHAIDAYLS AICGNLLYQN YPNLRPFFVY GQYKKFSSDP | |
| | DKEKAIFNKT RKFSFISQLL KNKSENSKEI AKKLKRAYQF KYMLVSRETE | |
| | TRDQEMFKMT VYPRFSHDTV KAPRNLIPKK MGMSPDIYGG YTNNSDAYMV | |
| | IVRIDKKKGT EYKILGIPTR ELVNLKKAEK EDHYKSYLKE ILTPRILYNK | |
| | NGKRDKKITS FEIVKSKIPY KQVIQDGDKK FMLGSSTYVY NAKQLTLSTE | |
| | SMKAITNNFD KDSDENDALI KAYDEILDKV DKYLPLFDIN KFREKLHSGR | |
| | EKFIKLSLED KKDTILKVLE GLHDNAVMTK IPTIGLSTPL GFMQFPNGVI | |
| | LSENAKLIYQ SPTGLFKKSV KISDL | |
| Mycoplasma | MNNSIKSKPE VTIGLDLGVG SVGWAIVDNE TNIIHHLGSR LFSQAKTAED | (SEQ |
| gallisepticum | RRSFRGVRRL IRRRKYKLKR FVNLIWKYNS YFGFKNKEDI LNNYQEQQKL | ID NO: |
| str. F | HNTVLNLKSE ALNAKIDPKA LSWILHDYLK NRGHFYEDNR DFNVYPTKEL | 39) |
| AJN60022.1 | AKYFDKYGYY KGIIDSKEDN DNKLEEELTK YKFSNKHWLE EVKKVLSNQT | |
| GI: 757015978 | GLPEKFKEEY ESLFSYVRNY SEGPGSINSV SPYGIYHLDE KEGKVVQKYN | |
| WP_ | NIWDKTIGKC NIFPDEYRAP KNSPIAMIFN EINELSTIRS YSIYLTGWFI | |
| 014574789.1 | NQEFKKAYLN KLLDLLIKTN GEKPIDARQF KKLREETIAE SIGKETLKDV | |
| | ENEEKLEKED HKWKLKGLKL NTNGKIQYND LSSLAKFVHK LKQHLKLDFL | |
| | LEDQYATLDK INFLQSLFVY LGKHLRYSNR VDSANLKEFS DSNKLFERIL | |
| | QKQKDGLFKL FEQTDKDDEK ILAQTHSLST KAMLLAITRM TNLDNDEDNQ | |
| | KNNDKGWNFE AIKNFDQKFI DITKKNNNLS LKQNKRYLDD RFINDAILSP | |
| | GVKRILREAT KVFNAILKQF SEEYDVTKVV IELARELSEE KELENTKNYK | |
| | KLIKKNGDKI SEGLKALGIS EDEIKDILKS PTKSYKFLLW LQQDHIDPYS | |
| | LKEIAFDDIF TKTEKFEIDH IIPYSISFDD SSSNKLLVLA ESNQAKSNQT | |
| | PYEFISSGNA GIKWEDYEAY CRKFKDGDSS LLDSTQRSKK FAKMMKTDTS | |
| | SKYDIGFLAR NLNDTRYATI VFRDALEDYA NNHLVEDKPM FKVVCINGSV | |
| | TSFLRKNFDD SSYAKKDRDK NIHHAVDASI ISIFSNETKT LFNQLTQFAD | |
| | YKLFKNTDGS WKKIDPKTGV VTEVTDENWK QIRVRNQVSE IAKVIEKYIQ | |
| | DSNIERKARY SRKIENKTNI SLFNDTVYSA KKVGYEDQIK RKNLKTLDIH | |
| | ESAKENKNSK VKRQFVYRKL VNVSLLNNDK LADLFAEKED ILMYRANPWV | |
| | INLAEQIFNE YTENKKIKSQ NVFEKYMLDL TKEFPEKFSE FLVKSMLRNK | |
| | TAIIYDDKKN IVHRIKRLKM LSSELKENKL SNVIIRSKNQ SGTKLSYQDT | |
| | INSLALMIMR SIDPTAKKQY IRVPLNTLNL HLGDHDFDLH NMDAYLKKPK | |
| | FVKYLKANEI GDEYKPWRVL TSGTLLIHKK DKKLMYISSF QNLNDVIEIK | |
| | NLIETEYKEN DDSDSKKKKK ANRFLMTLST ILNDYILLDA KDNFDILGLS | |
| | KNRIDEILNS KLGLDKIVK | |
| AJN60023.1 | MRILGFDIGI NSIGWAFVEN DELKDCGVRI FTKAENPKNK ESLALPRRNA | (SEQ |
| GI: 757015979 | RSSRRRLKRR KARLIAIKRI LAKELKLNYK DYVAADGELP KAYEGSLASV | ID NO: |
| | YELRYKALTQ NLETKDLARV ILHIAKHRGY MNKNEKKSND AKKGKILSAL | 430) |
| | KNNALKLENY QSVGEYFYKE FFQKYKKNTK NFIKIRNTKD NYNNCVLSSD | |
| | LEKELKLILE KQKEFGYNYS EDFINEILKV AFFQRPLKDF SHLVGACTFF | |
| | EEEKRACKNS YSAWEFVALT KIINEIKSLE KISGEIVPTQ TINEVLNLIL | |
| | DKGSITYKKF RSCINLHESI SFKSLKYDKE NAENAKLIDF RKLVEFKKAL | |
| | GVHSLSRQEL DQISTHITLI KDNVKLKTVL EKYNLSNEQI NNLLEIEFND | |
| | YINLSFKALG MILPLMREGK RYDEACEIAN LKPKTVDEKK DFLPAFCDSI | |
| | FAHELSNPVV NRAISEYRKV LNALLKKYGK VHKIHLELAR DVGLSKKARE | |
| | KIEKEQKENQ AVNAWALKEC ENIGLKASAK NILKLKLWKE QKEICIYSGN | |
| | KISIEHLKDE KALEVDHIYP YSRSFDDSFI NKVLVFTKEN QEKLNKTPFE | |
| | AFGKNIEKWS KIQTLAQNLP YKKKNKILDE NFKDKQQEDF ISRNLNDTRY | |
| | IATLIAKYTK EYLNFLLLSE NENANLKSGE KGSKIHVQTI SGMLTSVLRH | |
| | TWGFDKKDRN NHLHHALDAI IVAYSTNSII KAFSDFRKNQ ELLKARFYAK | |
| | ELTSDNYKHQ VKFFEPFKSF REKILSKIDE IFVSKPPRKR ARRALHKDTF | |
| | HSENKIIDKC SYNSKEGLQI ALSCGRVRKI GTKYVENDTI VRVDIFKKQN | |
| | KFYAIPIYAM DFALGILPNK IVITGKDKNN NPKQWQTIDE SYEFCFSLYK | |
| | NDLILLQKKN MQEPEFAYYN DFSISTSSIC VEKHDNKFEN LTSNQKLLFS | |
| | NAKEGSVKVE SLGIQNLKVF EKYIITPLGD KIKADFQPRE NISLKTSKKY | |
| | GLR | |
| AJN60025.1 | MSDLVLGLDI GIGSVGVGIL NKVTGEIIHK NSRIFPAAQA ENNLVRRTNR | (SEQ |
| GI: 757015981 | QGRRLARRKK HRRVRLNRLF EESGLITDFT KISINLNPYQ LRVKGLTDEL | ID NO: |
| | SNEELFIALK NMVKHRGISY LDDASDDGNS SVGDYAQIVK ENSKQLETKT | 41) |
| | PGQIQLERYQ TYGQLRGDFT VEKDGKKHRL INVFPTSAYR SEALRILQTQ | |
| | QEFNPQITDE FINRYLEILT GKRKYYHGPG NEKSRTDYGR YRTSGETLDN | |
| | IFGILIGKCT FYPDEFRAAK ASYTAQEFNL LNDLNNLTVP TETKKLSKEQ | |
| | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARETNEDDEK KAIQKIQKAN KDEKDAAMLK AANQYNGKAE | |
| | LPHSVFHGHK QLATKIRLWH QQGERCLYTG KTISIHDLIN NSNQFEVDHI | |
| | LPLSITFDDS LANKVLVYAT ANQEKGQRTP YQALDSMDDA WSFRELKAFV | |
| | RESKTLSNKK KEYLLTEEDI SKFDVRKKFI ERNLVDTRYA SRVVLNALQE | |
| | HFRAHKIDTK VSVVRGQFTS QLRRHWGIEK TRDTYHHHAV DALIIAASSQ | |
| | LNLWKKQKNT LVSYSEDQLL DIETGELISD DEYKESVFKA PYQHFVDTLK | |
| | SKEFEDSILF SYQVDSKFNR KISDATIYAT RQAKVGKDKA DETYVLGKIK | |
| | DIYTQDGYDA FMKIYKKDKS KFLMYRHDPQ TFEKVIEPIL ENYPNKQINE | |
| | KGKEVPCNPF LKYKEEHGYI RKYSKKGNGP EIKSLKYYDS KLGNHIDITP | |
| | KDSNNKVVLQ SVSPWRADVY FNKTTGKYEI LGLKYADLQF EKGTGTYKIS | |
| | QEKYNDIKKK EGVDSDSEFK FTLYKNDLLL VKDTETKEQQ LFRFLSRTMP | |
| | KQKHYVELKP YDKQKFEGGE ALIKVLGNVA NSGQCKKGLG KSNISIYKVR | |
| | TDVLGNQHII KNEGDKPKLM | |
| WP_00266404 | MKHILGLDLG TNSIGWALIE RNIEEKYGKI IGMGSRIVPM GAELSKFEQG | (SEQ |
| 8.1 | QAQTKNADRR TNRGARRLNK RYKQRRNKLI YILQKLDMLP SQIKLKEDFS | ID NO: |
| Bergeyella | DPNKIDKITI LPISKKQEQL TAFDLVSLRV KALTEKVGLE DLGKIIYKYN | 42) |
| zoohelcum | QLRGYAGGSL EPEKEDIFDE EQSKDKKNKS FIAFSKIVFL GEPQEEIFKN | |
| ATCC 43767 | KKLNRRAIIV ETEEGNFEGS TFLENIKVGD SLELLINISA SKSGDTITIK | |
| | LPNKTNWRKK MENIENQLKE KSKEMGREFY ISEFLLELLK ENRWAKIRNN | |
| | TILRARYESE FEAIWNEQVK HYPFLENLDK KTLIEIVSFI FPGEKESQKK | |
| | YRELGLEKGL KYIIKNQVVF YQRELKDQSH LISDCRYEPN EKAIAKSHPV | |
| | FQEYKVWEQI NKLIVNTKIE AGTNRKGEKK YKYIDRPIPT ALKEWIFEEL | |
| | QNKKEITFSA IFKKLKAEFD LREGIDFLNG MSPKDKLKGN ETKLQLQKSL | |
| | GELWDVLGLD SINRQIELWN ILYNEKGNEY DLTSDRTSKV LEFINKYGNN | |
| | IVDDNAEETA IRISKIKFAR AYSSLSLKAV ERILPLVRAG KYFNNDFSQQ | |
| | LQSKILKLLN ENVEDPFAKA AQTYLDNNQS VLSEGGVGNS IATILVYDKH | |
| | TAKEYSHDEL YKSYKEINLL KQGDLRNPLV EQIINEALVL IRDIWKNYGI | |
| | KPNEIRVELA RDLKNSAKER ATIHKRNKDN QTINNKIKET LVKNKKELSL | |
| | ANIEKVKLWE AQRHLSPYTG QPIPLSDLFD KEKYDVDHII PISRYFDDSF | |
| | TNKVISEKSV NQEKANRTAM EYFEVGSLKY SIFTKEQFIA HVNEYFSGVK | |
| | RKNLLATSIP EDPVQRQIKD TQYIAIRVKE ELNKIVGNEN VKTTTGSITD | |
| | YLRNHWGLTD KFKLLLKERY EALLESEKFL EAEYDNYKKD FDSRKKEYEE | |
| | KEVLFEEQEL TREEFIKEYK ENYIRYKKNK LIIKGWSKRI DHRHHAIDAL | |
| | IVACTEPAHI KRLNDLNKVL QDWLVEHKSE FMPNFEGSNS ELLEEILSLP | |
| | ENERTEIFTQ IEKFRAIEMP WKGFPEQVEQ KLKEIIISHK PKDKLLLQYN | |
| | KAGDRQIKLR GQLHEGTLYG ISQGKEAYRI PLTKFGGSKF ATEKNIQKIV | |
| | SPFLSGFIAN HLKEYNNKKE EAFSAEGIMD LNNKLAQYRN EKGELKPHTP | |
| | ISTVKIYYKD PSKNKKKKDE EDLSLQKLDR EKAFNEKLYV KTGDNYLFAV | |
| | LEGEIKTKKT SQIKRLYDII SFFDATNFLK EEFRNAPDKK TFDKDLLFRQ | |
| | YFEERNKAKL LFTLKQGDFV YLPNENEEVI LDKESPLYNQ YWGDLKERGK | |
| | NIYVVQKFSK KQIYFIKHTI ADIIKKDVEF GSQNCYETVE GRSIKENCFK | |
| | LEIDRLGNIV KVIKR | |
| CBK78998.1 | MKQEYFLGLD MGTGSLGWAV TDSTYQVMRK HGKALWGTRL FESASTAEER | (SEQ |
| Coprococcus | RMFRTARRRL DRRNWRIQVL QEIFSEEISK VDPGFFLRMK ESKYYPEDKR | ID NO: |
| catus GD/7 | DAEGNCPELP YALFVDDNYT DKNYHKDYPT IYHLRKMLME TTEIPDIRLV | 43) |
| | YLVLHHMMKH RGHFLLSGDI SQIKEFKSTF EQLIQNIQDE ELEWHISLDD | |
| | AAIQFVEHVL KDRNLTRSTK KSRLIKQLNA KSACEKAILN LLSGGTVKLS | |
| | DIFNNKELDE SERPKVSFAD SGYDDYIGIV EAELAEQYYI IASAKAVYDW | |
| | SVLVEILGNS VSISEAKIKV YQKHQADLKT LKKIVRQYMT KEDYKRVFVD | |
| | TEEKLNNYSA YIGMTKKNGK KVDLKSKQCT QADFYDFLKK NVIKVIDHKE | |
| | ITQEIESEIE KENFLPKQVT KDNGVIPYQV HDYELKKILD NLGTRMPFIK | |
| | ENAEKIQQLF EFRIPYYVGP LNRVDDGKDG KFTWSVRKSD ARIYPWNFTE | |
| | VIDVEASAEK FIRRMTNKCT YLVGEDVLPK DSLVYSKFMV LNELNNLRLN | |
| | GEKISVELKQ RIYEELFCKY RKVTRKKLER YLVIEGIAKK GVEITGIDGD | |
| | FKASLTAYHD FKERLTDVQL SQRAKEAIVL NVVLFGDDKK LLKQRLSKMY | |
| | PNLTTGQLKG ICSLSYQGWG RLSKTFLEEI TVPAPGTGEV WNIMTALWQT | |
| | NDNLMQLLSR NYGFTNEVEE FNTLKKETDL SYKTVDELYV SPAVKRQIWQ | |
| | TLKVVKEIQK VMGNAPKRVF VEMAREKQEG KRSDSRKKQL VELYRACKNE | |
| | ERDWITELNA QSDQQLRSDK LFLYYIQKGR CMYSGETIQL DELWDNTKYD | |
| | IDHIYPQSKT MDDSLNNRVL VKKNYNAIKS DTYPLSLDIQ KKMMSFWKML | |
| | QQQGFITKEK YVRLVRSDEL SADELAGFIE RQIVETRQST KAVATILKEA | |
| | LPDTEIVYVK AGNVSNFRQT YELLKVREMN DLHHAKDAYL NIVVGNAYFV | |
| | KFTKNAAWFI RNNPGRSYNL KRMFEFDIER SGEIAWKAGN KGSIVTVKKV | |
| | MQKNNILVTR KAYEVKGGLF DQQIMKKGKG QVPIKGNDER LADIEKYGGY | |
| | NKAAGTYFML VKSLDKKGKE IRTIEFVPLY LKNQIEINHE SAIQYLAQER | |
| | GLNSPEILLS KIKIDTLFKV DGFKMWLSGR TGNQLIFKGA NQLILSHQEA | |
| | AILKGVVKYV NRKNENKDAK LSERDGMTEE KLLQLYDTFL DKLSNTVYSI | |
| | RLSAQIKTLT EKRAKFIGLS NEDQCIVLNE ILHMFQCQSG SANLKLIGGP | |
| | GSAGILVMNN NITACKQISV INQSPTGIYE KEIDLIKL | |
| WP_00223516 | MAAFKPNPIN YILGLDIGIA SVGWAMVEID EDENPICLID LGVRVFERAE | (SEQ |
| 2.1 | VPKTGDSLAM ARRLARSVRR LTRRRAHRLL RARRLLKREG VLQAADFDEN | ID NO: |
| Neisseria | GLIKSLPNTP WQLRAAALDR KLTPLEWSAV LLHLIKHRGY LSQRKNEGET | 44) |
| meningitidis | ADKELGALLK GVADNAHALQ TGDFRTPAEL ALNKFEKESG HIRNQRGDYS | |
| Z2491 | HTFSRKDLQA ELILLFEKQK EFGNPHVSGG LKEGIETLLM TQRPALSGDA | |
| | VQKMLGHCTF EPAEPKAAKN TYTAERFIWL TKLNNLRILE QGSERPLTDT | |
| | ERATLMDEPY RKSKLTYAQA RKLLGLEDTA FFKGLRYGKD NAEASTLMEM | |
| | KAYHAISRAL EKEGLKDKKS PLNLSPELQD EIGTAFSLFK TDEDITGRLK | |
| | DRIQPEILEA LLKHISFDKF VQISLKALRR IVPLMEQGKR YDEACAEIYG | |
| | DHYGKKNTEE KIYLPPIPAD EIRNPVVLRA LSQARKVING VVRRYGSPAR | |
| | IHIETAREVG KSFKDRKEIE KRQEENRKDR EKAAAKFREY FPNFVGEPKS | |
| | KDILKLRLYE QQHGKCLYSG KEINLGRLNE KGYVEIDHAL PFSRTWDDSF | |
| | NNKVLVLGSE NQNKGNQTPY EYFNGKDNSR EWQEFKARVE TSRFPRSKKQ | |
| | RILLQKFDED GFKERNLNDT RYVNRFLCQF VADRMRLTGK GKKRVFASNG | |
| | QITNLLRGFW GLRKVRAEND RHHALDAVVV ACSTVAMQQK ITRFVRYKEM | |
| | NAFDGKTIDK ETGEVLHQKT HFPQPWEFFA QEVMIRVFGK PDGKPEFEEA | |
| | DTPEKLRTLL AEKLSSRPEA VHEYVTPLFV SRAPNRKMSG QGHMETVKSA | |
| | KRLDEGVSVL RVPLTQLKLK DLEKMVNRER EPKLYEALKA RLEAHKDDPA | |
| | KAFAEPFYKY DKAGNRTQQV KAVRVEQVQK TGVWVRNHNG IADNATMVRV | |
| | DVFEKGDKYY LVPIYSWQVA KGILPDRAVV QGKDEEDWQL IDDSFNFKFS | |
| | LHPNDLVEVI TKKARMFGYF ASCHRGTGNI NIRIHDLDHK IGKNGILEGI | |
| | GVKTALSFQK YQIDELGKEI RPCRLKKRPP VR | |
| WP_ | MQKNINTKQN HIYIKQAQKI KEKLGDKPYR IGLDLGVGSI GFAIVSMEEN | (SEQ |
| 012414420.1 | DGNVLLPKEI IMVGSRIFKA SAGAADRKLS RGQRNNHRHT RERMRYLWKV | ID NO: |
| Elusimicrobium | LAEQKLALPV PADLDRKENS SEGETSAKRF LGDVLQKDIY ELRVKSLDER | 45) |
| minutum | LSLQELGYVL YHIAGHRGSS AIRTFENDSE EAQKENTENK KIAGNIKRLM | |
| Pei191 | AKKNYRTYGE YLYKEFFENK EKHKREKISN AANNHKFSPT RDLVIKEAEA | |
| | ILKKQAGKDG FHKELTEEYI EKLTKAIGYE SEKLIPESGF CPYLKDEKRL | |
| | PASHKLNEER RLWETLNNAR YSDPIVDIVT GEITGYYEKQ FTKEQKQKLF | |
| | DYLLTGSELT PAQTKKLLGL KNTNFEDIIL QGRDKKAQKI KGYKLIKLES | |
| | MPFWARLSEA QQDSFLYDWN SCPDEKLLTE KLSNEYHLTE EEIDNAFNEI | |
| | VLSSSYAPLG KSAMLIILEK IKNDLSYTEA VEEALKEGKL TKEKQAIKDR | |
| | LPYYGAVLQE STQKIIAKGF SPQFKDKGYK TPHTNKYELE YGRIANPVVH | |
| | QTLNELRKLV NEIIDILGKK PCEIGLETAR ELKKSAEDRS KLSREQNDNE | |
| | SNRNRIYEIY IRPQQQVIIT RRENPRNYIL KFELLEEQKS QCPFCGGQIS | |
| | PNDIINNQAD IEHLFPIAES EDNGRNNLVI SHSACNADKA KRSPWAAFAS | |
| | AAKDSKYDYN RILSNVKENI PHKAWRFNQG AFEKFIENKP MAARFKTDNS | |
| | YISKVAHKYL ACLFEKPNII CVKGSLTAQL RMAWGLQGLM IPFAKQLITE | |
| | KESESFNKDV NSNKKIRLDN RHHALDAIVI AYASRGYGNL LNKMAGKDYK | |
| | INYSERNWLS KILLPPNNIV WENIDADLES FESSVKTALK NAFISVKHDH | |
| | SDNGELVKGT MYKIFYSERG YTLTTYKKLS ALKLTDPQKK KTPKDFLETA | |
| | LLKFKGRESE MKNEKIKSAI ENNKRLFDVI QDNLEKAKKL LEEENEKSKA | |
| | EGKKEKNIND ASIYQKAISL SGDKYVQLSK KEPGKFFAIS KPTPTTTGYG | |
| | YDTGDSLCVD LYYDNKGKLC GEIIRKIDAQ QKNPLKYKEQ GFTLFERIYG | |
| | GDILEVDFDI HSDKNSFRNN TGSAPENRVF IKVGTFTEIT NNNIQIWFGN | |
| | IIKSTGGQDD SFTINSMQQY NPRKLILSSC GFIKYRSPIL KNKEG | |
| WP_00910577 | MIMKLEKWRL GLDLGTNSIG WSVFSLDKDN SVQDLIDMGV RIFSDGRDPK | (SEQ |
| 7.1 | TKEPLAVARR TARSQRKLIY RRKLRRKQVF KFLQEQGLFP KTKEECMTLK | ID NO: |
| Treponema sp. | SLNPYELRIK ALDEKLEPYE LGRALFNLAV RRGFKSNRKD GSREEVSEKK | 46) |
| JC4 | SPDEIKTQAD MQTHLEKAIK ENGCRTITEF LYKNQGENGG IRFAPGRMTY | |
| | YPTRKMYEEE FNLIRSKQEK YYPQVDWDDI YKAIFYQRPL KPQQRGYCIY | |
| | ENDKERTFKA MPCSQKLRIL QDIGNLAYYE GGSKKRVELN DNQDKVLYEL | |
| | LNSKDKVTFD QMRKALCLAD SNSFNLEENR DFLIGNPTAV KMRSKNRFGK | |
| | LWDEIPLEEQ DLIIETIITA DEDDAVYEVI KKYDLTQEQR DFIVKNTILQ | |
| | SGTSMLCKEV SEKLVKRLEE IADLKYHEAV ESLGYKFADQ TVEKYDLLPY | |
| | YGKVLPGSTM EIDLSAPETN PEKHYGKISN PTVHVALNQT RVVVNALIKE | |
| | YGKPSQIAIE LSRDLKNNVE KKAEIARKQN QRAKENIAIN DTISALYHTA | |
| | FPGKSFYPNR NDRMKYRLWS ELGLGNKCIY CGKGISGAEL FTKEIEIEHI | |
| | LPFSRTLLDA ESNLTVAHSS CNAFKAERSP FEAFGTNPSG YSWQEIIQRA | |
| | NQLKNTSKKN KFSPNAMDSF EKDSSFIARQ LSDNQYIAKA ALRYLKCLVE | |
| | NPSDVWTTNG SMTKLLRDKW EMDSILCRKF TEKEVALLGL KPEQIGNYKK | |
| | NRFDHRHHAI DAVVIGLTDR SMVQKLATKN SHKGNRIEIP EFPILRSDLI | |
| | EKVKNIVVSF KPDHGAEGKL SKETLLGKIK LHGKETFVCR ENIVSLSEKN | |
| | LDDIVDEKIK SKVKDYVAKH KGQKIEAVLS DFSKENGIKK VRCVNRVQTP | |
| | IEITSGKISR YLSPEDYFAA VIWEIPGEKK TFKAQYIRRN EVEKNSKGLN | |
| | VVKPAVLENG KPHPAAKQVC LLHKDDYLEF SDKGKMYFCR IAGYAATNNK | |
| | LDIRPVYAVS YCADWINSTN ETMLTGYWKP TPTQNWVSVN VLFDKQKARL | |
| | VTVSPIGRVF RK | |
| WP_00246084 | MNQKFILGLD IGITSVGYGL IDYETKNIID AGVRLFPEAN VENNEGRRSK | (SEQ |
| 8.1 | RGSRRLKRRR IHRLERVKKL LEDYNLLDQS QIPQSTNPYA IRVKGLSEAL | ID NO: |
| Staphylococcus | SKDELVIALL HIAKRRGIHK IDVIDSNDDV GNELSTKEQL NKNSKLLKDK | 47) |
| lugdunensis | FVCQIQLERM NEGQVRGEKN RFKTADIIKE IIQLLNVQKN FHQLDENFIN | |
| M23590 | KYIELVEMRR EYFEGPGKGS PYGWEGDPKA WYETLMGHCT YFPDELRSVK | |
| | YAYSADLFNA LNDLNNLVIQ RDGLSKLEYH EKYHIIENVF KQKKKPTLKQ | |
| | IANEINVNPE DIKGYRITKS GKPQFTEFKL YHDLKSVLFD QSILENEDVL | |
| | DQIAEILTIY QDKDSIKSKL TELDILLNEE DKENIAQLTG YTGTHRLSLK | |
| | CIRLVLEEQW YSSRNQMEIF THLNIKPKKI NLTAANKIPK AMIDEFILSP | |
| | VVKRTFGQAI NLINKIIEKY GVPEDIIIEL ARENNSKDKQ KFINEMQKKN | |
| | ENTRKRINEI IGKYGNQNAK RLVEKIRLHD EQEGKCLYSL ESIPLEDLLN | |
| | NPNHYEVDHI IPRSVSFDNS YHNKVLVKQS ENSKKSNLTP YQYFNSGKSK | |
| | LSYNQFKQHI LNLSKSQDRI SKKKKEYLLE ERDINKFEVQ KEFINRNLVD | |
| | TRYATRELTN YLKAYFSANN MNVKVKTING SFTDYLRKVW KFKKERNHGY | |
| | KHHAEDALII ANADFLFKEN KKLKAVNSVL EKPEIESKQL DIQVDSEDNY | |
| | SEMFIIPKQV QDIKDFRNFK YSHRVDKKPN RQLINDTLYS TRKKDNSTYI | |
| | VQTIKDIYAK DNTTLKKQFD KSPEKFLMYQ HDPRTFEKLE VIMKQYANEK | |
| | NPLAKYHEET GEYLTKYSKK NNGPIVKSLK YIGNKLGSHL DVTHQFKSST | |
| | KKLVKLSIKP YRFDVYLTDK GYKFITISYL DVLKKDNYYY IPEQKYDKLK | |
| | LGKAIDKNAK FIASFYKNDL IKLDGEIYKI IGVNSDTRNM IELDLPDIRY | |
| | KEYCELNNIK GEPRIKKTIG KKVNSIEKLT TDVLGNVFTN TQYTKPQLLF | |
| | KRGN | |
| WP_ | MTKPYSIGLD IGTNSVGWAV TTDNYKVPSK KMKVLGNTSK KYIKKNLLGV | (SEQ |
| 011681470.1 | LLFDSGITAE GRRLKRTARR RYTRRRNRIL YLQEIFSTEM ATLDDAFFQR | ID NO: |
| Streptococcus | LDDSFLVPDD KRDSKYPIFG NLVEEKAYHD EFPTIYHLRK YLADSTKKAD | 48) |
| thermophilus | LRLVYLALAH MIKYRGHFLI EGEFNSKNND IQKNFQDFLD TYNAIFESDL | |
| LMD-9 | SLENSKQLEE IVKDKISKLE KKDRILKLFP GEKNSGIFSE FLKLIVGNQA | |
| | DFRKCFNLDE KASLHFSKES YDEDLETLLG YIGDDYSDVF LKAKKLYDAI | |
| | LLSGFLTVTD NETEAPLSSA MIKRYNEHKE DLALLKEYIR NISLKTYNEV | |
| | FKDDTKNGYA GYIDGKTNQE DFYVYLKKLL AEFEGADYFL EKIDREDFLR | |
| | KQRTFDNGSI PYQIHLQEMR AILDKQAKFY PFLAKNKERI EKILTFRIPY | |
| | YVGPLARGNS DFAWSIRKRN EKITPWNFED VIDKESSAEA FINRMTSFDL | |
| | YLPEEKVLPK HSLLYETFNV YNELTKVRFI AESMRDYQFL DSKQKKDIVR | |
| | LYFKDKRKVT DKDIIEYLHA IYGYDGIELK GIEKQFNSSL STYHDLLNII | |
| | NDKEFLDDSS NEAIIEEIIH TLTIFEDREM IKQRLSKFEN IFDKSVLKKL | |
| | SRRHYTGWGK LSAKLINGIR DEKSGNTILD YLIDDGISNR NFMQLIHDDA | |
| | LSFKKKIQKA QIIGDEDKGN IKEVVKSLPG SPAIKKGILQ SIKIVDELVK | |
| | VMGGRKPESI VVEMARENQY TNQGKSNSQQ RLKRLEKSLK ELGSKILKEN | |
| | IPAKLSKIDN NALQNDRLYL YYLQNGKDMY TGDDLDIDRL SNYDIDHIIP | |
| | QAFLKDNSID NKVLVSSASN RGKSDDVPSL EVVKKRKTFW YQLLKSKLIS | |
| | QRKFDNLTKA ERGGLSPEDK AGFIQRQLVE TRQITKHVAR LLDEKFNNKK | |
| | DENNRAVRTV KIITLKSTLV SQFRKDFELY KVREINDFHH AHDAYLNAVV | |
| | ASALLKKYPK LEPEFVYGDY PKYNSFRERK SATEKVYFYS NIMNIFKKSI | |
| | SLADGRVIER PLIEVNEETG ESVWNKESDL ATVRRVLSYP QVNVVKKVEE | |
| | QNHGLDRGKP KGLFNANLSS KPKPNSNENL VGAKEYLDPK KYGGYAGISN | |
| | SFTVLVKGTI EKGAKKKITN VLEFQGISIL DRINYRKDKL NFLLEKGYKD | |
| | IELIIELPKY SLFELSDGSR RMLASILSTN NKRGEIHKGN QIFLSQKFVK | |
| | LLYHAKRISN TINENHRKYV ENHKKEFEEL FYYILEFNEN YVGAKKNGKL | |
| | LNSAFQSWQN HSIDELCSSF IGPTGSERKG LFELTSRGSA ADFEFLGVKI | |
| | PRYRDYTPSS LLKDATLIHQ SVTGLYETRI DLAKLGEG | |
| WP_ | MKRILGLDLG TNSIGWALVN EAENKDERSS IVKLGVRVNP LTVDELTNFE | (SEQ |
| 009293010.1 | KGKSITTNAD RTLKRGMRRN LQRYKLRRET LTEVLKEHKL ITEDTILSEN | ID NO: |
| Bacteroides | GNRTTFETYR LRAKAVTEEI SLEEFARVLL MINKKRGYKS SRKAKGVEEG | 49) |
| fragilis NCTC | TLIDGMDIAR ELYNNNLTPG ELCLQLLDAG KKFLPDFYRS DLQNELDRIW | |
| 9343 Cas9 | EKQKEYYPEI LTDVLKEELR GKKRDAVWAI CAKYFVWKEN YTEWNKEKGK | |
| | TEQQEREHKL EGIYSKRKRD EAKRENLQWR VNGLKEKLSL EQLVIVFQEM | |
| | NTQINNSSGY LGAISDRSKE LYFNKQTVGQ YQMEMLDKNP NASLRNMVFY | |
| | RQDYLDEFNM LWEKQAVYHK ELTEELKKEI RDIIIFYQRR LKSQKGLIGF | |
| | CEFESRQIEV DIDGKKKIKT VGNRVISRSS PLFQEFKIWQ ILNNIEVTVV | |
| | GKKRKRRKLK ENYSALFEEL NDAEQLELNG SRRLCQEEKE LLAQELFIRD | |
| | KMTKSEVLKL LFDNPQELDL NFKTIDGNKT GYALFQAYSK MIEMSGHEPV | |
| | DFKKPVEKVV EYIKAVFDLL NWNTDILGFN SNEELDNQPY YKLWHLLYSF | |
| | EGDNTPTGNG RLIQKMTELY GFEKEYATIL ANVSFQDDYG SLSAKAIHKI | |
| | LPHLKEGNRY DVACVYAGYR HSESSLTREE IANKVLKDRL MLLPKNSLHN | |
| | PVVEKILNQM VNVINVIIDI YGKPDEIRVE LARELKKNAK EREELTKSIA | |
| | QTTKAHEEYK TLLQTEFGLT NVSRTDILRY KLYKELESCG YKTLYSNTYI | |
| | SREKLFSKEF DIEHIIPQAR LFDDSFSNKT LEARSVNIEK GNKTAYDFVK | |
| | EKFGESGADN SLEHYLNNIE DLFKSGKISK TKYNKLKMAE QDIPDGFIER | |
| | DLRNTQYIAK KALSMLNEIS HRVVATSGSV TDKLREDWQL IDVMKELNWE | |
| | KYKALGLVEY FEDRDGRQIG RIKDWTKRND HRHHAMDALT VAFTKDVFIQ | |
| | YFNNKNASLD PNANEHAIKN KYFQNGRAIA PMPLREFRAE AKKHLENTLI | |
| | SIKAKNKVIT GNINKTRKKG GVNKNMQQTP RGQLHLETIY GSGKQYLTKE | |
| | EKVNASFDMR KIGTVSKSAY RDALLKRLYE NDNDPKKAFA GKNSLDKQPI | |
| | WLDKEQMRKV PEKVKIVTLE AIYTIRKEIS PDLKVDKVID VGVRKILIDR | |
| | LNEYGNDAKK AFSNLDKNPI WLNKEKGISI KRVTISGISN AQSLHVKKDK | |
| | DGKPILDENG RNIPVDFVNT GNNHHVAVYY RPVIDKRGQL VVDEAGNPKY | |
| | ELEEVVVSFF EAVTRANLGL PIIDKDYKTT EGWQFLFSMK QNEYFVFPNE | |
| | KTGFNPKEID LLDVENYGLI SPNLFRVQKF SLKNYVFRHH LETTIKDTSS | |
| | ILRGITWIDF RSSKGLDTIV KVRVNHIGQI VSVGEY | |
| AOL40912.1 | METQTSNQLI TSHLKDYPKQ DYFVGLDIGT NSVGWAVTNT SYELLKFHSH | (SEQ |
| Veillonella | KMWGSRLFEE GESAVTRRGF RSMRRRLERR KLRLKLLEEL FADAMAQVDS | ID NO: |
| atypica ACS- | TFFIRLHESK YHYEDKTTGH SSKHILFIDE DYTDQDYFTE YPTIYHLRKD | 50) |
| 134-V-Col7a | LMENGTDDIR KLFLAVHHIL KYRGNFLYEG ATFNSNAFTF EDVLKQALVN | |
| | ITFNCFDTNS AISSISNILM ESGKTKSDKA KAIERLVDTY TVFDEVNTPD | |
| | KPQKEQVKED KKTLKAFANL VLGLSANLID LFGSVEDIDD DLKKLQIVGD | |
| | TYDEKRDELA KVWGDEIHII DDCKSVYDAI ILMSIKEPGL TISQSKVKAF | |
| | DKHKEDLVIL KSLLKLDRNV YNEMFKSDKK GLHNYVHYIK QGRTEETSCS | |
| | REDFYKYTKK IVEGLADSKD KEYILNEIEL QTLLPLQRIK DNGVIPYQLH | |
| | LEELKVILDK CGPKFPFLHT VSDGFSVTEK LIKMLEFRIP YYVGPLNTHH | |
| | NIDNGGFSWA VRKQAGRVTP WNFEEKIDRE KSAAAFIKNL TNKCTYLFGE | |
| | DVLPKSSLLY SEFMLLNELN NVRIDGKALA QGVKQHLIDS IFKQDHKKMT | |
| | KNRIELFLKD NNYITKKHKP EITGLDGEIK NDLTSYRDMV RILGNNFDVS | |
| | MAEDIITDIT IFGESKKMLR QTLRNKFGSQ LNDETIKKLS KLRYRDWGRL | |
| | SKKLLKGIDG CDKAGNGAPK TIIELMRNDS YNLMEILGDK FSFMECIEEE | |
| | NAKLAQGQVV NPHDIIDELA LSPAVKRAVW QALRIVDEVA HIKKALPSRI | |
| | FVEVARTNKS EKKKKDSRQK RLSDLYSAIK KDDVLQSGLQ DKEFGALKSG | |
| | LANYDDAALR SKKLYLYYTQ MGRCAYTGNI IDLNQLNTDN YDIDHIYPRS | |
| | LTKDDSFDNL VLCERTANAK KSDIYPIDNR IQTKQKPFWA FLKHQGLISE | |
| | RKYERLTRIA PLTADDLSGF IARQLVETNQ SVKATTTLLR RLYPDIDVVF | |
| | VKAENVSDFR HNNNFIKVRS LNHHHHAKDA YLNIVVGNVY HEKFTRNFRL | |
| | FFKKNGANRT YNLAKMFNYD VICTNAQDGK AWDVKTSMNT VKKMMASNDV | |
| | RVTRRLLEQS GALADATIYK ASVAAKAKDG AYIGMKTKYS VFADVTKYGG | |
| | MTKIKNAYSI IVQYTGKKGE EIKEIVPLPI YLINRNATDI ELIDYVKSVI | |
| | PKAKDISIKY RKLCINQLVK VNGFYYYLGG KTNDKIYIDN AIELVVPHDI | |
| | ATYIKLLDKY DLLRKENKTL KASSITTSIY NINTSTVVSL SNKVGIDVFD | |
| | YFMSKLRTPL YMKMKGNKVD ELSSTGRSKF IKMTLEEQSI YLLEVLNLLT | |
| | NSKTTFDVKP LGITGSRSTI GVKIHNLDEF KIINESITGL YSNEVTIV | |
| WP_ | MKYSIGLDIG IASVGWSVIN KDKERIEDMG VRIFQKAENP KDGSSLASSR | (SEQ |
| 013389026.1 | REKRGSRRRN RRKKHRLDRI KNILCESGLV KKNEIEKIYK NAYLKSPWEL | ID NO: |
| Ilyobacter | RAKSLEAKIS NKEIAQILLH IAKRRGFKSF RKTDRNADDT GKLLSGIQEN | 51) |
| polytropus | KKIMEEKGYL TIGDMVAKDP KFNTHVRNKA GSYLFSFSRK LLEDEVRKIQ | |
| DSM 2926 | AKQKELGNTH FTDDVLEKYI EVFNSQRNFD EGPSKPSPYY SEIGQIAKMI | |
| | GNCTFESSEK RTAKNTWSGE RFVFLQKLNN FRIVGLSGKR PLTEEERDIV | |
| | EKEVYLKKEV RYEKLRKILY LKEEERFGDL NYSKDEKQDK KTEKTKFISL | |
| | IGNYTIKKLN LSEKLKSEIE EDKSKLDKII EILTFNKSDK TIESNLKKLE | |
| | LSREDIEILL SEEFSGTLNL SLKAIKKILP YLEKGLSYNE ACEKADYDYK | |
| | NNGIKFKRGE LLPVVDKDLI ANPVVLRAIS QTRKVVNAII RKYGTPHTIH | |
| | VEVARDLAKS YDDRQTIIKE NKKRELENEK TKKFISEEFG IKNVKGKLLL | |
| | KYRLYQEQEG RCAYSRKELS LSEVILDESM TDIDHIIPYS RSMDDSYSNK | |
| | VLVLSGENRK KSNLLPKEYF DRQGRDWDTF VLNVKAMKIH PRKKSNLLKE | |
| | KFTREDNKDW KSRALNDTRY ISRFVANYLE NALEYRDDSP KKRVFMIPGQ | |
| | LTAQLRARWR LNKVRENGDL HHALDAAVVA VTDQKAINNI SNISRYKELK | |
| | NCKDVIPSIE YHADEETGEV YFEEVKDTRF PMPWSGFDLE LQKRLESENP | |
| | REEFYNLLSD KRYLGWFNYE EGFIEKLRPV FVSRMPNRGV KGQAHQETIR | |
| | SSKKISNQIA VSKKPLNSIK LKDLEKMQGR DTDRKLYEAL KNRLEEYDDK | |
| | PEKAFAEPFY KPTNSGKRGP LVRGIKVEEK QNVGVYVNGG QASNGSMVRI | |
| | DVFRKNGKFY TVPIYVHQTL LKELPNRAIN GKPYKDWDLI DGSFEFLYSF | |
| | YPNDLIEIEF GKSKSIKNDN KLTKTEIPEV NLSEVLGYYR GMDTSTGAAT | |
| | IDTQDGKIQM RIGIKTVKNI KKYQVDVLGN VYKVKREKRQ TF | |
| WP_00586426 | MKKIVGLDLG TNSIGWALIN AYINKEHLYG IEACGSRIIP MDAAILGNFD | (SEQ |
| 3.1 | KGNSISQTAD RTSYRGIRRL RERHLLRRER LHRILDLLGF LPKHYSDSLN | ID NO: |
| Parabacteroides | RYGKFLNDIE CKLPWVKDET GSYKFIFQES FKEMLANFTE HHPILIANNK | 52) |
| sp. 20_3 | KVPYDWTIYY LRKKALTQKI SKEELAWILL NFNQKRGYYQ LRGEEEETPN | |
| | KLVEYYSLKV EKVEDSGERK GKDTWYNVHL ENGMIYRRTS NIPLDWEGKT | |
| | KEFIVTTDLE ADGSPKKDKE GNIKRSFRAP KDDDWTLIKK KTEADIDKIK | |
| | MTVGAYIYDT LLQKPDQKIR GKLVRTIERK YYKNELYQIL KTQSEFHEEL | |
| | RDKQLYIACL NELYPNNEPR RNSISTRDFC HLFIEDIIFY QRPLKSKKSL | |
| | IDNCPYEENR YIDKESGEIK HASIKCIAKS HPLYQEFRLW QFIVNLRIYR | |
| | KETDVDVTQE LLPTEADYVT LFEWLNEKKE IDQKAFFKYP PFGFKKTTSN | |
| | YRWNYVEDKP YPCNETHAQI IARLGKAHIP KAFLSKEKEE TLWHILYSIE | |
| | DKQEIEKALH SFANKNNLSE EFIEQFKNFP PFKKEYGSYS AKAIKKLLPL | |
| | MRMGKYWSIE NIDNGTRIRI NKIIDGEYDE NIRERVRQKA INLTDITHFR | |
| | ALPLWLACYL VYDRHSEVKD IVKWKTPKDI DLYLKSFKQH SLRNPIVEQV | |
| | ITETLRTVRD IWQQVGHIDE IHIELGREMK NPADKRARMS QQMIKNENTN | |
| | LRIKALLTEF LNPEFGIENV RPYSPSQQDL LRIYEEGVLN SILELPEDIG | |
| | IILGKFNQTD TLKRPTRSEI LRYKLWLEQK YRSPYTGEMI PLSKLFTPAY | |
| | EIEHIIPQSR YFDDSLSNKV ICESEINKLK DRSLGYEFIK NHHGEKVELA | |
| | FDKPVEVLSV EAYEKLVHES YSHNRSKMKK LLMEDIPDQF IERQLNDSRY | |
| | ISKVVKSLLS NIVREENEQE AISKNVIPCT GGITDRLKKD WGINDVWNKI | |
| | VLPRFIRLNE LTESTRFTSI NTNNTMIPSM PLELQKGFNK KRIDHRHHAM | |
| | DAIIIACANR NIVNYLNNVS ASKNTKITRR DLQTLLCHKD KTDNNGNYKW | |
| | VIDKPWETFT QDTLTALQKI TVSFKQNLRV INKTTNHYQH YENGKKIVSN | |
| | QSKGDSWAIR KSMHKETVHG EVNLRMIKTV SFNEALKKPQ AIVEMDLKKK | |
| | ILAMLELGYD TKRIKNYFEE NKDTWQDINP SKIKVYYFTK ETKDRYFAVR | |
| | KPIDTSFDKK KIKESITDTG IQQIMLRHLE TKDNDPTLAF SPDGIDEMNR | |
| | NILILNKGKK HQPIYKVRVY EKAEKFTVGQ KGNKRTKFVE AAKGTNLFFA | |
| | IYETEEIDKD TKKVIRKRSY STIPLNVVIE RQKQGLSSAP EDENGNLPKY | |
| | ILSPNDLVYV PTQEEINKGE VVMPIDRDRI YKMVDSSGIT ANFIPASTAN | |
| | LIFALPKATA EIYCNGENCI QNEYGIGSPQ SKNQKAITGE MVKEICFPIK | |
| | VDRLGNIIQV GSCILTN | |
| GAP01010.1 | MVYDVGLDIG TGSVGWVALD ENGKLARAKG KNLVGVRLFD TAQTAADRRG | (SEQ |
| Fructobacillus | FRTTRRRLSR RKWRLRLLDE LFSAEINEID SSFFQRLKYS YVHPKDEENK | ID NO: |
| fructosus | AHYYGGYLFP TEEETKKFHR SYPTIYHLRQ ELMAQPNKRF DIREIYLAIH | 53) |
| KCTC 3544 | HLVKYRGHFL SSQEKITIGS TYNPEDLANA IEVYADEKGL SWELNNPEQL | |
| | TEIISGEAGY GLNKSMKADE ALKLFEFDNN QDKVAIKTLL AGLTGNQIDF | |
| | AKLFGKDISD KDEAKLWKLK LDDEALEEKS QTILSQLTDE EIELFHAVVQ | |
| | AYDGFVLIGL LNGADSVSAA MVQLYDQHRE DRKLLKSLAQ KAGLKHKRFS | |
| | EIYEQLALAT DEATIKNGIS TARELVEESN LSKEVKEDTL RRLDENEFLP | |
| | KQRTKANSVI PHQLHLAELQ KILQNQGQYY PFLLDTFEKE DGQDNKIEEL | |
| | LRFRIPYYVG PLVTKKDVEH AGGDADNHWV ERNEGFEKSR VTPWNFDKVF | |
| | NRDKAARDFI ERLTGNDTYL IGEKTLPQNS LRYQLFTVLN ELNNVRVNGK | |
| | KFDSKTKADL INDLFKARKT VSLSALKDYL KAQGKGDVTI TGLADESKFN | |
| | SSLSSYNDLK KTFDAEYLEN EDNQETLEKI IEIQTVFEDS KIASRELSKL | |
| | PLDDDQVKKL SQTHYTGWGR LSEKLLDSKI IDERGQKVSI LDKLKSTSQN | |
| | FMSIINNDKY GVQAWITEQN TGSSKLTFDE KVNELTTSPA NKRGIKQSFA | |
| | VLNDIKKAMK EEPRRVYLEF AREDQTSVRS VPRYNQLKEK YQSKSLSEEA | |
| | KVLKKTLDGN KNKMSDDRYF LYFQQQGKDM YTGRPINFER LSQDYDIDHI | |
| | IPQAFTKDDS LDNRVLVSRP ENARKSDSFA YTDEVQKQDG SLWTSLLKSG | |
| | FINRKKYERL TKAGKYLDGQ KTGFIARQLV ETRQIIKNVA SLIEGEYENS | |
| | KAVAIRSEIT ADMRLLVGIK KHREINSFHH AFDALLITAA GQYMQNRYPD | |
| | RDSTNVYNEF DRYTNDYLKN LRQLSSRDEV RRLKSFGFVV GTMRKGNEDW | |
| | SEENTSYLRK VMMFKNILTT KKTEKDRGPL NKETIFSPKS GKKLIPLNSK | |
| | RSDTALYGGY SNVYSAYMTL VRANGKNLLI KIPISIANQI EVGNLKINDY | |
| | IVNNPAIKKF EKILISKLPL GQLVNEDGNL IYLASNEYRH NAKQLWLSTT | |
| | DADKIASISE NSSDEELLEA YDILTSENVK NRFPFFKKDI DKLSQVRDEF | |
| | LDSDKRIAVI QTILRGLQID AAYQAPVKII SKKVSDWHKL QQSGGIKLSD | |
| | NSEMIYQSAT GIFETRVKIS DLL | |
| Bacillus smithii | MNYKMGLDIG IASVGWAVIN LDLKRIEDLG VRIFDKAEHP QNGESLALPR | (SEQ |
| WP_00335419 | RIARSARRRL RRRKHRLERI RRLLVSENVL TKEEMNLLFK QKKQIDVWQL | ID NO: |
| 6.1 | RVDALERKLN NDELARVLLH LAKRRGFKSN RKSERNSKES SEFLKNIEEN | 54) |
| | QSILAQYRSV GEMIVKDSKF AYHKRNKLDS YSNMIARDDL EREIKLIFEK | |
| | QREFNNPVCT ERLEEKYLNI WSSQRPFASK EDIEKKVGFC TFEPKEKRAP | |
| | KATYTFQSFI VWEHINKLRL VSPDETRALT EIERNLLYKQ AFSKNKMTYY | |
| | DIRKLLNLSD DIHFKGLLYD PKSSLKQIEN IRFLELDSYH KIRKCIENVY | |
| | GKDGIRMFNE TDIDTFGYAL TIFKDDEDIV AYLQNEYITK NGKRVSNLAN | |
| | KVYDKSLIDE LLNLSFSKFA HLSMKAIRNI LPYMEQGEIY SKACELAGYN | |
| | FTGPKKKEKA LLLPVIPNIA NPVVMRALTQ SRKVVNAIIK KYGSPVSIHI | |
| | ELARDLSHSF DERKKIQKDQ TENRKKNETA IKQLIEYELT KNPTGLDIVK | |
| | FKLWSEQQGR CMYSLKPIEL ERLLEPGYVE VDHILPYSRS LDDSYANKVL | |
| | VLTKENREKG NHTPVEYLGL GSERWKKFEK FVLANKQFSK KKKQNLLRLR | |
| | YEETEEKEFK ERNLNDTRYI SKFFANFIKE HLKFADGDGG QKVYTINGKI | |
| | TAHLRSRWDF NKNREESDLH HAVDAVIVAC ATQGMIKKIT EFYKAREQNK | |
| | ESAKKKEPIF PQPWPHFADE LKARLSKFPQ ESIEAFALGN YDRKKLESLR | |
| | PVFVSRMPKR SVTGAAHQET LRRCVGIDEQ SGKIQTAVKT KLSDIKLDKD | |
| | GHFPMYQKES DPRTYEAIRQ RLLEHNNDPK KAFQEPLYKP KKNGEPGPVI | |
| | RTVKIIDTKN KVVHLDGSKT VAYNSNIVRT DVFEKDGKYY CVPVYTMDIM | |
| | KGTLPNKAIE ANKPYSEWKE MTEEYTFQFS LFPNDLVRIV LPREKTIKTS | |
| | TNEEIIIKDI FAYYKTIDSA TGGLELISHD RNFSLRGVGS KTLKRFEKYQ | |
| | VDVLGNIHKV KGEKRVGLAA PTNQKKGKTV DSLQSVSD | |
| Mycoplasma | MEKKRKVTLG FDLGIASVGW AIVDSETNQV YKLGSRLFDA PDTNLERRTQ | (SEQ |
| canis PG 14 | RGTRRLLRRR KYRNQKFYNL VKRTEVFGLS SREAIENRFR ELSIKYPNII | ID NO: |
| EIE39736.1 | ELKTKALSQE VCPDEIAWIL HDYLKNRGYF YDEKETKEDF DQQTVESMPS | 55) |
| WP_004794730.1 | YKLNEFYKKY GYFKGALSQP TESEMKDNKD LKEAFFFDFS NKEWLKEINY | |
| | FFNVQKNILS ETFIEEFKKI FSFTRDISKG PGSDNMPSPY GIFGEFGDNG | |
| | QGGRYEHIWD KNIGKCSIFT NEQRAPKYLP SALIFNFLNE LANIRLYSTD | |
| | KKNIQPLWKL SSVDKLNILL NLFNLPISEK KKKLTSTNIN DIVKKESIKS | |
| | IMISVEDIDM IKDEWAGKEP NVYGVGLSGL NIEESAKENK FKFQDLKILN | |
| | VLINLLDNVG IKFEFKDRND IIKNLELLDN LYLFLIYQKE SNNKDSSIDL | |
| | FIAKNESLNI ENLKLKLKEF LLGAGNEFEN HNSKTHSLSK KAIDEILPKL | |
| | LDNNEGWNLE AIKNYDEEIK SQIEDNSSLM AKQDKKYLND NFLKDAILPP | |
| | NVKVTFQQAI LIFNKIIQKF SKDFEIDKVV IELAREMTQD QENDALKGIA | |
| | KAQKSKKSLV EERLEANNID KSVFNDKYEK LIYKIFLWIS QDFKDPYTGA | |
| | QISVNEIVNN KVEIDHIIPY SLCFDDSSAN KVLVHKQSNQ EKSNSLPYEY | |
| | IKQGHSGWNW DEFTKYVKRV FVNNVDSILS KKERLKKSEN LLTASYDGYD | |
| | KLGFLARNLN DTRYATILFR DQLNNYAEHH LIDNKKMFKV IAMNGAVTSF | |
| | IRKNMSYDNK LRLKDRSDFS HHAYDAAIIA LFSNKTKTLY NLIDPSLNGI | |
| | ISKRSEGYWV IEDRYTGEIK ELKKEDWTSI KNNVQARKIA KEIEEYLIDL | |
| | DDEVFFSRKT KRKTNRQLYN ETIYGIATKT DEDGITNYYK KEKFSILDDK | |
| | DIYLRLLRER EKFVINQSNP EVIDQIIEII ESYGKENNIP SRDEAINIKY | |
| | TKNKINYNLY LKQYMRSLTK SLDQFSEEFI NQMIANKTFV LYNPTKNTTR | |
| | KIKFLRLVND VKINDIRKNQ VINKFNGKNN EPKAFYENIN SLGAIVFKNS | |
| | ANNFKTLSIN TQIAIFGDKN WDIEDFKTYN MEKIEKYKEI YGIDKTYNFH | |
| | SFIFPGTILL DKQNKEFYYI SSIQTVRDII EIKFLNKIEF KDENKNQDTS | |
| | KTPKRLMFGI KSIMNNYEQV DISPFGINKK IFE | |
| Odoribacter | METTLGIDLG TNSIGLALVD QEEHQILYSG VRIFPEGINK DTIGLGEKEE | (SEQ |
| laneus YIT | SRNATRRAKR QMRRQYFRKK LRKAKLLELL IAYDMCPLKP EDVRRWKNWD | ID NO: |
| EHP49880.1 | KQQKSTVRQF PDTPAFREWL KQNPYELRKQ AVTEDVTRPE LGRILYQMIQ | 56) |
| | RRGFLSSRKG KEEGKIFTGK DRMVGIDETR KNLQKQTLGA YLYDIAPKNG | |
| | EKYRFRTERV RARYTLRDMY IREFEIIWQR QAGHLGLAHE QATRKKNIFL | |
| | EGSATNVRNS KLITHLQAKY GRGHVLIEDT RITVTFQLPL KEVLGGKIEI | |
| | EEEQLKFKSN ESVLFWQRPL RSQKSLLSKC VFEGRNFYDP VHQKWIIAGP | |
| | TPAPLSHPEF EEFRAYQFIN NIIYGKNEHL TAIQREAVFE LMCTESKDFN | |
| | FEKIPKHLKL FEKFNFDDTT KVPACTTISQ LRKLFPHPVW EEKREEIWHC | |
| | FYFYDDNTLL FEKLQKDYAL QTNDLEKIKK IRLSESYGNV SLKAIRRINP | |
| | YLKKGYAYST AVLLGGIRNS FGKRFEYFKE YEPEIEKAVC RILKEKNAEG | |
| | EVIRKIKDYL VHNRFGFAKN DRAFQKLYHH SQAITTQAQK ERLPETGNLR | |
| | NPIVQQGLNE LRRTVNKLLA TCREKYGPSF KFDHIHVEMG RELRSSKTER | |
| | EKQSRQIREN EKKNEAAKVK LAEYGLKAYR DNIQKYLLYK EIEEKGGTVC | |
| | CPYTGKTLNI SHTLGSDNSV QIEHIIPYSI SLDDSLANKT LCDATFNREK | |
| | GELTPYDFYQ KDPSPEKWGA SSWEEIEDRA FRLLPYAKAQ RFIRRKPQES | |
| | NEFISRQLND TRYISKKAVE YLSAICSDVK AFPGQLTAEL RHLWGLNNIL | |
| | QSAPDITFPL PVSATENHRE YYVITNEQNE VIRLFPKQGE TPRTEKGELL | |
| | LTGEVERKVF RCKGMQEFQT DVSDGKYWRR IKLSSSVTWS PLFAPKPISA | |
| | DGQIVLKGRI EKGVFVCNQL KQKLKTGLPD GSYWISLPVI SQTFKEGESV | |
| | NNSKLTSQQV QLFGRVREGI FRCHNYQCPA SGADGNFWCT LDTDTAQPAF | |
| | TPIKNAPPGV GGGQIILTGD VDDKGIFHAD DDLHYELPAS LPKGKYYGIF | |
| | TVESCDPTLI PIELSAPKTS KGENLIEGNI WVDEHTGEVR FDPKKNREDQ | |
| | RHHAIDAIVI ALSSQSLFQR LSTYNARREN KKRGLDSTEH FPSPWPGFAQ | |
| | DVRQSVVPLL VSYKQNPKTL CKISKTLYKD GKKIHSCGNA VRGQLHKETV | |
| | YGQRTAPGAT EKSYHIRKDI RELKTSKHIG KVVDITIRQM LLKHLQENYH | |
| | IDITQEFNIP SNAFFKEGVY RIFLPNKHGE PVPIKKIRMK EELGNAERLK | |
| | DNINQYVNPR NNHHVMIYQD ADGNLKEEIV SFWSVIERQN QGQPIYQLPR | |
| | EGRNIVSILQ INDTFLIGLK EEEPEVYRND LSTLSKHLYR VQKLSGMYYT | |
| | FRHHLASTLN NEREEFRIQS LEAWKRANPV KVQIDEIGRI TFLNGPLC | |
| Akkermansia | MSRSLTFSFD IGYASIGWAV IASASHDDAD PSVCGCGTVL FPKDDCQAFK | (SEQ |
| muciniphila | RREYRRLRRN IRSRRVRIER IGRLLVQAQI ITPEMKETSG HPAPFYLASE | ID NO: |
| ATCC BAA- | ALKGHRTLAP IELWHVLRWY AHNRGYDNNA SWSNSLSEDG GNGEDTERVK | 57) |
| 835 | HAQDLMDKHG TATMAETICR ELKLEEGKAD APMEVSTPAY KNLNTAFPRL | |
| WP_01242103 | IVEKEVRRIL ELSAPLIPGL TAEIIELIAQ HHPLTTEQRG VLLQHGIKLA | |
| 4.1 | RRYRGSLLFG QLIPRFDNRI ISRCPVTWAQ VYEAELKKGN SEQSARERAE | |
| | KLSKVPTANC PEFYEYRMAR ILCNIRADGE PLSAEIRREL MNQARQEGKL | |
| | TKASLEKAIS SRLGKETETN VSNYFTLHPD SEEALYLNPA VEVLQRSGIG | |
| | QILSPSVYRI AANRLRRGKS VTPNYLLNLL KSRGESGEAL EKKIEKESKK | |
| | KEADYADTPL KPKYATGRAP YARTVLKKWV EEILDGEDPT RPARGEAHPD | |
| | GELKAHDGCL YCLLDTDSSV NQHQKERRLD TMTNNHLVRH RMLILDRLLK | |
| | DLIQDFADGQ KDRISRVCVE VGKELTTFSA MDSKKIQREL TLRQKSHTDA | |
| | VNRLKRKLPG KALSANLIRK CRIAMDMNWT CPFTGATYGD HELENLELEH | |
| | IVPHSFRQSN ALSSLVLTWP GVNRMKGQRT GYDFVEQEQE NPVPDKPNLH | |
| | ICSLNNYREL VEKLDDKKGH EDDRRRKKKR KALLMVRGLS HKHQSQNHEA | |
| | MKEIGMTEGM MTQSSHLMKL ACKSIKTSLP DAHIDMIPGA VTAEVRKAWD | |
| | VFGVFKELCP EAADPDSGKI LKENLRSLTH LHHALDACVL GLIPYIIPAH | |
| | HNGLLRRVLA MRRIPEKLIP QVRPVANQRH YVLNDDGRMM LRDLSASLKE | |
| | NIREQLMEQR VIQHVPADMG GALLKETMQR VLSVDGSGED AMVSLSKKKD | |
| | GKKEKNQVKA SKLVGVFPEG PSKLKALKAA IEIDGNYGVA LDPKPVVIRH | |
| | IKVFKRIMAL KEQNGGKPVR ILKKGMLIHL TSSKDPKHAG VWRIESIQDS | |
| | KGGVKLDLQR AHCAVPKNKT HECNWREVDL ISLLKKYQMK RYPTSYTGTP | |
| | R | |
| Dinoroseobacter | MRLGLDIGTS SIGWWLYETD GAGSDARITG WDGGVRIFS DGRDPKSGAS | (SEQ |
| shibae DFL | LAVDRRAARA MRRRRDRYLR RRATLMKVLA ETGLMPADPA EAKALEALDP | ID NO: |
| 12 = DSM | FALRAAGLDE PLPLPHLGRA LFHLNQRRGF KSNRKTDRGD NESGKIKDAT | 58) |
| 16493 | ARLDMEMMAN GARTYGEFLH KRRQKATDPR HVPSVRTRLS IANRGGPDGK | |
| WP_01217707 | EEAGYDFYPD RRHLEEEFHK LWAAQGAHHP ELTETLRDLL FEKIFFQRPL | |
| 9.1 | KEPEVGLCLF SGHHGVPPKD PRLPKAHPLT QRRVLYETVN QLRVTADGRE | |
| | ARPLTREERD QVIHALDNKK PTKSLSSMVL KLPALAKVLK LRDGERFTLE | |
| | TGVRDAIACD PLRASPAHPD RFGPRWSILD ADAQWEVISR IRRVQSDAEH | |
| | AALVDWLTEA HGLDRAHAEA TAHAPLPDGY GRLGLTATTR ILYQLTADWV | |
| | TYADAVKACG WHHSDGRTGE CFDRLPYYGE VLERHVIPGS YHPDDDDITR | |
| | FGRITNPTVH IGLNQLRRLV NRIIETHGKP HQIVVELARD LKKSEEQKRA | |
| | DIKRIRDTTE AAKKRSEKLE ELEIEDNGRN RMLLRLWEDL NPDDAMRRFC | |
| | PYTGTRISAA MIFDGSCDVD HILPYSRTLD DSFPNRTLCL REANRQKRNQ | |
| | TPWQAWGDTP HWHAIAANLK NLPENKRWRF APDAMTRFEG ENGFLDRALK | |
| | DTQYLARISR SYLDTLFTKG GHVWVVPGRF TEMLRRHWGL NSLLSDAGRG | |
| | AVKAKNRTDH RHHAIDAAVI AATDPGLLNR ISRAAGQGEA AGQSAELIAR | |
| | DTPPPWEGFR DDLRVRLDRI IVSHRADHGR IDHAARKQGR DSTAGQLHQE | |
| | TAYSIVDDIH VASRTDLLSL KPAQLLDEPG RSGQVRDPQL RKALRVATGG | |
| | KTGKDFENAL RYFASKPGPY QAIRRVRIIK PLQAQARVPV PAQDPIKAYQ | |
| | GGSNHLFEIW RLPDGEIEAQ VITSFEAHTL EGEKRPHPAA KRLLRVHKGD | |
| | MVALERDGRR VVGHVQKMDI ANGLFIVPHN EANADTRNND KSDPFKWIQI | |
| | GARPAIASGI RRVSVDEIGR LRDGGTRPI | |
| Wolinella | MIERILGVDL GISSLGWAIV EYDKDDEAAN RIIDCGVRLF TAAETPKKKE | (SEQ |
| succinogenes | SPNKARREAR GIRRVLNRRR VRMNMIKKLF LRAGLIQDVD LDGEGGMFYS | ID NO: |
| DSM 1740 | KANRADVWEL RHDGLYRLLK GDELARVLIH IAKHRGYKFI GDDEADEESG | 59) |
| WP_01113928 | KVKKAGVVLR QNFEAAGCRT VGEWLWRERG ANGKKRNKHG DYEISIHRDL | |
| 9.1 | LVEEVEAIFV AQQEMRSTIA TDALKAAYRE IAFFVRPMQR IEKMVGHCTY | |
| | FPEERRAPKS APTAEKFIAI SKFFSTVIID NEGWEQKIIE RKTLEELLDF | |
| | AVSREKVEFR HLRKFLDLSD NEIFKGLHYK GKPKTAKKRE ATLFDPNEPT | |
| | ELEFDKVEAE KKAWISLRGA AKLREALGNE FYGRFVALGK HADEATKILT | |
| | YYKDEGQKRR ELTKLPLEAE MVERLVKIGF SDFLKLSLKA IRDILPAMES | |
| | GARYDEAVLM LGVPHKEKSA ILPPLNKTDI DILNPTVIRA FAQFRKVANA | |
| | LVRKYGAFDR VHFELAREIN TKGEIEDIKE SQRKNEKERK EAADWIAETS | |
| | FQVPLTRKNI LKKRLYIQQD GRCAYTGDVI ELERLFDEGY CEIDHILPRS | |
| | RSADDSFANK VLCLARANQQ KTDRTPYEWF GHDAARWNAF ETRTSAPSNR | |
| | VRTGKGKIDR LLKKNFDENS EMAFKDRNLN DTRYMARAIK TYCEQYWVFK | |
| | NSHTKAPVQV RSGKLTSVLR YQWGLESKDR ESHTHHAVDA IIIAFSTQGM | |
| | VQKLSEYYRF KETHREKERP KLAVPLANFR DAVEEATRIE NTETVKEGVE | |
| | VKRLLISRPP RARVTGQAHE QTAKPYPRIK QVKNKKKWRL APIDEEKFES | |
| | FKADRVASAN QKNFYETSTI PRVDVYHKKG KFHLVPIYLH EMVLNELPNL | |
| | SLGTNPEAMD ENFFKFSIFK DDLISIQTQG TPKKPAKIIM GYFKNMHGAN | |
| | MVLSSINNSP CEGFTCTPVS MDKKHKDKCK LCPEENRIAG RCLQGFLDYW | |
| | SQEGLRPPRK EFECDQGVKF ALDVKKYQID PLGYYYEVKQ EKRLGTIPQM | |
| | RSAKKLVKK | |
| Parasutterella | MGKTHIIGVG LDLGGTYTGT FITSHPSDEA EHRDHSSAFT VVNSEKLSFS | (SEQ |
| excrementihominis | SKSRTAVRHR VRSYKGFDLR RRLLLLVAEY QLLQKKQTLA PEERENLRIA | ID NO: |
| YIT | LSGYLKRRGY ARTEAETDTS VLESLDPSVF SSAPSFTNFF NDSEPLNIQW | 60) |
| 11859 | EAIANSPETT KALNKELSGQ KEADFKKYIK TSFPEYSAKE ILANYVEGRR | |
| WP_00886484 | AILDASKYIA NLQSLGHKHR SKYLSDILQD MKRDSRITRL SEAFGSTDNL | |
| 3.1 | WRIIGNISNL QERAVRWYFN DAKFEQGQEQ LDAVKLKNVL VRALKYLRSD | |
| | DKEWSASQKQ IIQSLEQSGD VLDVLAGLDP DRTIPPYEDQ NNRRPPEDQT | |
| | LYLNPKALSS EYGEKWKSWA NKFAGAYPLL TEDLTEILKN TDRKSRIKIR | |
| | SDVLPDSDYR LAYILQRAFD RSIALDECSI RRTAEDFENG VVIKNEKLED | |
| | VLSGHQLEEF LEFANRYYQE TAKAKNGLWF PENALLERAD LHPPMKNKIL | |
| | NVIVGQALGV SPAEGTDFIE EIWNSKVKGR STVRSICNAI ENERKTYGPY | |
| | FSEDYKFVKT ALKEGKTEKE LSKKFAAVIK VLKMVSEVVP FIGKELRLSD | |
| | EAQSKFDNLY SLAQLYNLIE TERNGFSKVS LAAHLENAWR MTMTDGSAQC | |
| | CRLPADCVRP FDGFIRKAID RNSWEVAKRI AEEVKKSVDF TNGTVKIPVA | |
| | IEANSFNFTA SLTDLKYIQL KEQKLKKKLE DIQRNEENQE KRWLSKEERI | |
| | RADSHGICAY TGRPLDDVGE IDHIIPRSLT LKKSESIYNS EVNLIFVSAQ | |
| | GNQEKKNNIY LLSNLAKNYL AAVFGTSDLS QITNEIESTV LQLKAAGRLG | |
| | YFDLLSEKER ACARHALFLN SDSEARRAVI DVLGSRRKAS VNGTQAWFVR | |
| | SIFSKVRQAL AAWTQETGNE LIFDAISVPA ADSSEMRKRF AEYRPEFRKP | |
| | KVQPVASHSI DAMCIYLAAC SDPFKTKRMG SQLAIYEPIN FDNLFTGSCQ | |
| | VIQNTPRNFS DKTNIANSPI FKETIYAERF LDIIVSRGEI FIGYPSNMPF | |
| | EEKPNRISIG GKDPFSILSV LGAYLDKAPS SEKEKLTIYR VVKNKAFELF | |
| | SKVAGSKFTA EEDKAAKILE ALHFVTVKQD VAATVSDLIK SKKELSKDSI | |
| | ENLAKQKGCL KKVEYSSKEF KFKGSLIIPA AVEWGKVLWN VFKENTAEEL | |
| | KDENALRKAL EAAWPSSFGT RNLHSKAKRV FSLPVVATQS GAVRIRRKTA | |
| | FGDFVYQSQD TNNLYSSFPV KNGKLDWSSP IIHPALQNRN LTAYGYRFVD | |
| | HDRSISMSEF REVYNKDDLM RIELAQGTSS RRYLRVEMPG EKFLAWFGEN | |
| | SISLGSSFKF SVSEVFDNKI YTENAEFTKF LPKPREDNKH NGTIFFELVG | |
| | PRVIFNYIVG GAASSLKEIF SEAGKERS | |
| Streptococcus | MTKFNKNYSI GLDIGVSSVG YAVVTEDYRV PAFKFKVLGN TEKEKIKKNL | (SEQ |
| sanguinis | IGSTTFVSAQ PAKGTRVFRV NRRRIDRRNH RITYLRDIFQ KEIEKVDKNF | ID NO: |
| SK49 | YRRLDESFRV LGDKSEDLQI KQPFFGDKEL ETAYHKKYPT IYHLRKHLAD | 61) |
| WP_00293358 | ADKNSPVADI REVYMAISHI LKYRGHFLTL DKINPNNINM QNSWIDFIES | |
| 9.1 | CQEVFDLEIS DESKNIADIF KSSENRQEKV KKILPYFQQE LLKKDKSIFK | |
| | QLLQLLFGLK TKFKDCFELE EEPDLNFSKE NYDENLENFL GSLEEDFSDV | |
| | FAKLKVLRDT ILLSGMLTYT GATHARFSAT MVERYEEHRK DLQRFKFFIK | |
| | QNLSEQDYLD IFGRKTQNGF DVDKETKGYV GYITNKMVLT NPQKQKTIQQ | |
| | NFYDYISGKI TGIEGAEYFL NKISDGTFLR KLRTSDNGAI PNQIHAYELE | |
| | KIIERQGKDY PFLLENKDKL LSILTFKIPY YVGPLAKGSN SRFAWIKRAT | |
| | SSDILDDNDE DTRNGKIRPW NYQKLINMDE TRDAFITNLI GNDIILLNEK | |
| | VLPKRSLIYE EVMLQNELTR VKYKDKYGKA HFFDSELRQN IINGLFKNNS | |
| | KRVNAKSLIK YLSDNHKDLN AIEIVSGVEK GKSFNSTLKT YNDLKTIFSE | |
| | ELLDSEIYQK ELEEIIKVIT VFDDKKSIKN YLTKFFGHLE ILDEEKINQL | |
| | SKLRYSGWGR YSAKLLLDIR DEDTGFNLLQ FLRNDEENRN LTKLISDNTL | |
| | SFEPKIKDIQ SKSTIEDDIF DEIKKLAGSP AIKRGILNSI KIVDELVQII | |
| | GYPPHNIVIE MARENMTTEE GQKKAKTRKT KLESALKNIE NSLLENGKVP | |
| | HSDEQLQSEK LYLYYLQNGK DMYTLDKTGS PAPLYLDQLD QYEVDHIIPY | |
| | SFLPIDSIDN KVLTHRENNQ QKLNNIPDKE TVANMKPFWE KLYNAKLISQ | |
| | TKYQRLTTSE RTPDGVLTES MKAGFIERQL VETRQIIKHV ARILDNRFSD | |
| | TKIITLKSQL ITNFRNTFHI AKIRELNDYH HAHDAYLAVV VGQTLLKVYP | |
| | KLAPELIYGH HAHFNRHEEN KATLRKHLYS NIMRFFNNPD SKVSKDIWDC | |
| | NRDLPIIKDV IYNSQINFVK RTMIKKGAFY NQNPVGKFNK QLAANNRYPL | |
| | KTKALCLDTS IYGGYGPMNS ALSIIIIAER FNEKKGKIET VKEFHDIFII | |
| | DYEKFNNNPF QFLNDTSENG FLKKNNINRV LGFYRIPKYS LMQKIDGTRM | |
| | LFESKSNLHK ATQFKLTKTQ NELFFHMKRL LTKSNLMDLK SKSAIKESQN | |
| | FILKHKEEFD NISNQLSAFS QKMLGNTTSL KNLIKGYNER KIKEIDIRDE | |
| | TIKYFYDNFI KMFSFVKSGA PKDINDFFDN KCTVARMRPK PDKKLLNATL | |
| | IHQSITGLYE TRIDLSKLGE D | |
| Actinomyces | MLHCIAVIRV PPSEEPGFFE THADSCALCH HGCMTYAAND KAIRYRVGID | (SEQ |
| sp. oral taxon | VGLRSIGFCA VEVDDEDHPI RILNSVVHVH DAGTGGPGET ESLRKRSGVA | ID NO: |
| 180 str. F0310 | ARARRRGRAE KQRLKKLDVL LEELGWGVSS NELLDSHAPW HIRKRLVSEY | 62) |
| AOL41039.1 | IEDETERRQC LSVAMAHIAR HRGWRNSFSK VDTLLLEQAP SDRMQGLKER | |
| | VEDRTGLQFS EEVTQGELVA TLLEHDGDVT IRGFVRKGGK ATKVHGVLEG | |
| | KYMQSDLVAE LRQICRTQRV SETTFEKLVL SIFHSKEPAP SAARQRERVG | |
| | LDELQLALDP AAKQPRAERA HPAFQKFKVV ATLANMRIRE QSAGERSLTS | |
| | EELNRVARYL LNHTESESPT WDDVARKLEV PRHRLRGSSR ASLETGGGLT | |
| | YPPVDDTTVR VMSAEVDWLA DWWDCANDES RGHMIDAISN GCGSEPDDVE | |
| | DEEVNELISS ATAEDMLKLE LLAKKLPSGR VAYSLKTLRE VTAAILETGD | |
| | DLSQAITRLY GVDPGWVPTP APIEAPVGNP SVDRVLKQVA RWLKFASKRW | |
| | GVPQTVNIEH TREGLKSASL LEEERERWER FEARREIRQK EMYKRLGISG | |
| | PFRRSDQVRY EILDLQDCAC LYCGNEINFQ TFEVDHIIPR VDASSDSRRT | |
| | NLAAVCHSCN SAKGGLAFGQ WVKRGDCPSG VSLENAIKRV RSWSKDRLGL | |
| | TEKAMGKRKS EVISRLKTEM PYEEFDGRSM ESVAWMAIEL KKRIEGYFNS | |
| | DRPEGCAAVQ VNAYSGRLTA CARRAAHVDK RVRLIRLKGD DGHHKNRFDR | |
| | RNHAMDALVI ALMTPAIART IAVREDRREA QQLTRAFESW KNFLGSEERM | |
| | QDRWESWIGD VEYACDRLNE LIDADKIPVT ENLRLRNSGK LHADQPESLK | |
| | KARRGSKRPR PQRYVLGDAL PADVINRVTD PGLWTALVRA PGFDSQLGLP | |
| | ADLNRGLKLR GKRISADFPI DYFPTDSPAL AVQGGYVGLE FHHARLYRII | |
| | GPKEKVKYAL LRVCAIDLCG IDCDDLFEVE LKPSSISMRT ADAKLKEAMG | |
| | NGSAKQIGWL VLGDEIQIDP TKFPKQSIGK FLKECGPVSS WRVSALDTPS | |
| | KITLKPRLLS NEPLLKTSRV GGHESDLVVA ECVEKIMKKT GWVVEINALC | |
| | QSGLIRVIRR NALGEVRTSP KSGLPISLNL R | |
| Rhodovulum | MGIRFAFDLG TNSIGWAVWR TGPGVFGEDT AASLDGSGVL IFKDGRNPKD | (SEQ |
| sp. PH10 | GQSLATMRRV PRQSRKRRDR FVLRRRDLLA ALRKAGLFPV DVEEGRRLAA | ID NO: |
| WP_00838698 | TDPYHLRAKA LDESLTPHEM GRVIFHLNQR RGFRSNRKAD RQDREKGKIA | 63) |
| 3.1 | EGSKRLAETL AATNCRTLGE FLWSRHRGTP RTRSPTRIRM EGEGAKALYA | |
| | FYPTREMVRA EFERLWTAQS RFAPDLLTPE RHEEIAGILF RQRDLAPPKI | |
| | GCCTFEPSER RLPRALPSVE ARGIYERLAH LRITTGPVSD RGLTRPERDV | |
| | LASALLAGKS LTFKAVRKTL KILPHALVNF EEAGEKGLDG ALTAKLLSKP | |
| | DHYGAAWHGL SFAEKDTFVG KLLDEADEER LIRRLVTENR LSEDAARRCA | |
| | SIPLADGYGR LGRTANTEIL AALVEETDET GTVVTYAEAV RRAGERTGRN | |
| | WHHSDERDGV ILDRLPYYGE ILQRHVVPGS GEPEEKNEAA RWGRLANPTV | |
| | HIGLNQLRKV VNRLIAAHGR PDQIVVELAR ELKLNREQKE RLDRENRKNR | |
| | EENERRTAIL AEHGQRDTAE NKIRLRLFEE QARANAGIAL CPYTGRAIGI | |
| | AELFTSEVEI DHILPVSLTL DDSLANRVLC RREANREKRR QTPFQAFGAT | |
| | PAWNDIVARA AKLPPNKRWR FDPAALERFE REGGFLGRQL NETKYLSRLA | |
| | KIYLGKICDP DRVYVTPGTL TGLLRARWGL NSILSDSNFK NRSDHRHHAV | |
| | DAVVIGVLTR GMIQRIAHDA ARAEDQDLDR VFRDVPVPFE DFRDHVRERV | |
| | STITVAVKPE HGKGGALHED TSYGLVPDTD PNAALGNLVV RKPIRSLTAG | |
| | EVDRVRDRAL RARLGALAAP FRDESGRVRD AKGLAQALEA FGAENGIRRV | |
| | RILKPDASVV TIADRRTGVP YRAVAPGENH HVDIVQMRDG SWRGFAASVF | |
| | EVNRPGWRPE WEVKKLGGKL VMRLHKGDMV ELSDKDGQRR VKVVQQIEIS | |
| | ANRVRLSPHN DGGKLQDRHA DADDPFRWDL ATIPLLKDRG CVAVRVDPIG | |
| | WTLRRSNV | |
| Bifidobacterium | MSRKNYVDDY AISLDIGNAS VGWSAFTPNY RLVRAKGHEL IGVRLFDPAD | (SEQ |
| bifidum S17 | TAESRRMART TRRRYSRRRW RLRLLDALFD QALSEIDPSF LARRKYSWVH | ID NO: |
| WP_01336299 | PDDENNADCW YGSVLFDSNE QDKRFYEKYP TIYHLRKALM EDDSQHDIRE | 64) |
| 5.1 | IYLAIHHMVK YRGNFLVEGT LESSNAFKED ELLKLLGRIT RYEMSEGEQN | |
| | SDIEQDDENK LVAPANGQLA DALCATRGSR SMRVDNALEA LSAVNDLSRE | |
| | QRAIVKAIFA GLEGNKLDLA KIFVSKEFSS ENKKILGIYF NKSDYEEKCV | |
| | QIVDSGLLDD EEREFLDRMQ GQYNAIALKQ LLGRSTSVSD SKCASYDAHR | |
| | ANWNLIKLQL RTKENEKDIN ENYGILVGWK IDSGQRKSVR GESAYENMRK | |
| | KANVFFKKMI ETSDLSETDK NRLIHDIEED KLFPIQRDSD NGVIPHQLHQ | |
| | NELKQIIKKQ GKYYPFLLDA FEKDGKQINK IEGLLTFRVP YFVGPLVVPE | |
| | DLQKSDNSEN HWMVRKKKGE ITPWNFDEMV DKDASGRKFI ERLVGTDSYL | |
| | LGEPTLPKNS LLYQEYEVLN ELNNVRLSVR TGNHWNDKRR MRLGREEKTL | |
| | LCQRLFMKGQ TVTKRTAENL LRKEYGRTYE LSGLSDESKF TSSLSTYGKM | |
| | CRIFGEKYVN EHRDLMEKIV ELQTVFEDKE TLLHQLRQLE GISEADCALL | |
| | VNTHYTGWGR LSRKLLTTKA GECKISDDFA PRKHSIIEIM RAEDRNLMEI | |
| | ITDKQLGFSD WIEQENLGAE NGSSLMEVVD DLRVSPKVKR GIIQSIRLID | |
| | DISKAVGKRP SRIFLELADD IQPSGRTISR KSRLQDLYRN ANLGKEFKGI | |
| | ADELNACSDK DLQDDRLFLY YTQLGKDMYT GEELDLDRLS SAYDIDHIIP | |
| | QAVTQNDSID NRVLVARAEN ARKTDSFTYM PQIADRMRNF WQILLDNGLI | |
| | SRVKFERLTR QNEFSEREKE RFVQRSLVET RQIMKNVATL MRQRYGNSAA | |
| | VIGLNAELTK EMHRYLGFSH KNRDINDYHH AQDALCVGIA GQFAANRGFF | |
| | ADGEVSDGAQ NSYNQYLRDY LRGYREKLSA EDRKQGRAFG FIVGSMRSQD | |
| | EQKRVNPRTG EVVWSEEDKD YLRKVMNYRK MLVTQKVGDD FGALYDETRY | |
| | AATDPKGIKG IPFDGAKQDT SLYGGFSSAK PAYAVLIESK GKTRLVNVTM | |
| | QEYSLLGDRP SDDELRKVLA KKKSEYAKAN ILLRHVPKMQ LIRYGGGLMV | |
| | IKSAGELNNA QQLWLPYEEY CYFDDLSQGK GSLEKDDLKK LLDSILGSVQ | |
| | CLYPWHRFTE EELADLHVAF DKLPEDEKKN VITGIVSALH ADAKTANLSI | |
| | VGMTGSWRRM NNKSGYTFSD EDEFIFQSPS GLFEKRVTVG ELKRKAKKEV | |
| | NSKYRTNEKR LPTLSGASQP | |
| Barnesiella | MKNILGLDLG LSSIGWSVIR ENSEEQELVA MGSRVVSLTA AELSSFTQGN | (SEQ |
| intestinihominis | GVSINSQRTQ KRTQRKGYDR YQLRRTLLRN KLDTLGMLPD DSLSYLPKLQ | ID NO: |
| YIT 11860 | LWGLRAKAVT QRIELNELGR VLLHLNQKRG YKSIKSDFSG DKKITDYVKT | 65) |
| WP_ | VKTRYDELKE MRLTIGELFF RRLTENAFFR CKEQVYPRQA YVEEFDCIMN | |
| 008863245.1 | CQRKFYPDIL TDETIRCIRD EIIYYQRPLK SCKYLVSRCE FEKRFYLNAA | |
| | GKKTEAGPKV SPRTSPLFQV CRLWESINNI WKDRRNEIV FISAEQRAAL | |
| | FDFLNTHEKL KGSDLLKLLG LSKTYGYRLG EQFKTGIQGN KTRVEIERAL | |
| | GNYPDKKRLL QFNLQEESSS MVNTETGEII PMISLSFEQE PLYRLWHVLY | |
| | SIDDREQLQS VLRQKFGIDD DEVLERLSAI DLVKAGFGNK SSKAIRRILP | |
| | FLQLGMNYAE ACEAAGYNHS NNYTKAENEA RALLDRLPAI KKNELRQPWV | |
| | EKILNQMVNV VNALMEKYGR FDEIRVELAR ELKQSKEERS NTYKSINKNQ | |
| | RENEQIAKRI VEYGVPTRSR IQKYKMWEES KHCCIYCGQP VDVGDFLRGF | |
| | DVEVEHIIPK SLYFDDSFAN KVCSCRSCNK EKNNRTAYDY MKSKGEKALS | |
| | DYVERVNTMY TNNQISKTKW QNLLTPVDKI SIDFIDRQLR ESQYIARKAK | |
| | EILTSICYNV TATSGSVTSF LRHVWGWDTV LHDLNFDRYK KVGLTEVIEV | |
| | NHRGSVIRRE QIKDWSKRED HRHHAIDALT IACTKQAYIQ RLNNLRAEEG | |
| | PDFNKMSLER YIQSQPHFSV AQVREAVDRI LVSFRAGKRA VTPGKRYIRK | |
| | NRKRISVQSV LIPRGALSEE SVYGVIHVWE KDEQGHVIQK QRAVMKYPIT | |
| | SINREMLDKE KWVDKRIHRI LSGRLAQYND NPKEAFAKPV YIDKECRIPI | |
| | RTVRCFAKPA INTLVPLKKD DKGNPVAWVN PGNNHHVAIY RDEDGKYKER | |
| | TVTFWEAVDR CRVGIPAIVT QPDTIWDNIL QRNDISENVL ESLPDVKWQF | |
| | VLSLQQNEMF ILGMNEEDYR YAMDQQDYAL LNKYLYRVQK LSKSDYSFRY | |
| | HTETSVEDKY DGKPNLKLSM QMGKLKRVSI KSLLGLNPHK VHISVLGEIK | |
| | EIS | |
| Aminomonas | MIGEHVRGGC LFDDHWTPNW GAFRLPNTVR TFTKAENPKD GSSLAEPRRQ | (SEQ |
| paucivorans | ARGLRRRLRR KTQRLEDLRR LLAKEGVLSL SDLETLFRET PAKDPYQLRA | ID NO: |
| DSM 12260 | EGLDRPLSFP EWVRVLYHIT KHRGFQSNRR NPVEDGQERS RQEEEGKLLS | 66) |
| WP_ | GVGENERLLR EGGYRTAGEM LARDPKFQDH RRNRAGDYSH TLSRSLLLEE | |
| 006299850.1 | ARRLFQSQRT LGNPHASSNL EEAFLHLVAF QNPFASGEDI RNKAGHCSLE | |
| | PDQIRAPRRS ASAETFMLLQ KTGNLRLIHR RTGEERPLTD KEREQIHLLA | |
| | WKQEKVTHKT LRRHLEIPEE WLFTGLPYHR SGDKAEEKLF VHLAGIHEIR | |
| | KALDKGPDPA VWDTLRSRRD LLDSIADTLT FYKNEDEILP RLESLGLSPE | |
| | NARALAPLSF SGTAHLSLSA LGKLLPHLEE GKSYTQARAD AGYAAPPPDR | |
| | HPKLPPLEEA DWRNPVVFRA LTQTRKVVNA LVRRYGPPWC IHLETARELS | |
| | QPAKVRRRIE TEQQANEKKK QQAEREFLDI VGTAPGPGDL LKMRLWREQG | |
| | GFCPYCEEYL NPTRLAEPGY AEMDHILPYS RSLDNGWHNR VLVHGKDNRD | |
| | KGNRTPFEAF GGDTARWDRL VAWVQASHLS APKKRNLLRE DFGEEAEREL | |
| | KDRNLTDTRF ITKTAATLLR DRLTFHPEAP KDPVMTLNGR LTAFLRKQWG | |
| | LHKNRKNGDL HHALDAAVLA VASRSFVYRL SSHNAAWGEL PRGREAENGF | |
| | SLPYPAFRSE VLARLCPTRE EILLRLDQGG VGYDEAFRNG LRPVFVSRAP | |
| | SRRLRGKAHM ETLRSPKWKD HPEGPRTASR IPLKDLNLEK LERMVGKDRD | |
| | RKLYEALRER LAAFGGNGKK AFVAPFRKPC RSGEGPLVRS LRIFDSGYSG | |
| | VELRDGGEVY AVADHESMVR VDVYAKKNRF YLVPVYVADV ARGIVKNRAI | |
| | VAHKSEEEWD LVDGSFDFRF SLFPGDLVEI EKKDGAYLGY YKSCHRGDGR | |
| | LLLDRHDRMP RESDCGTFYV STRKDVLSMS KYQVDPLGEI RLVGSEKPPF | |
| | VL | |
| Ralstonia | MAEKQHRWGL DIGTNSIGWA VIALIEGRPA GLVATGSRIF SDGRNPKDGS | (SEQ |
| syzygii R24 | SLAVERRGPR QMRRRRDRYL RRRDRFMQAL INVGLMPGDA AARKALVTEN | ID NO: |
| CCA84553.1 | PYVLRQRGLD QALTLPEFGR ALFHLNQRRG FQSNRKTDRA TAKESGKVKN | 67) |
| | AIAAFRAGMG NARTVGEALA RRLEDGRPVR ARMVGQGKDE HYELYIAREW | |
| | IAQEFDALWA SQQRFHAEVL ADAARDRLRA ILLFQRKLLP VPVGKCFLEP | |
| | NQPRVAAALP SAQRFRLMQE LNHLRVMTLA DKRERPLSFQ ERNDLLAQLV | |
| | ARPKCGFDML RKIVFGANKE AYRFTIESER RKELKGCDTA AKLAKVNALG | |
| | TRWQALSLDE QDRLVCLLLD GENDAVLADA LREHYGLTDA QIDTLLGLSF | |
| | EDGHMRLGRS ALLRVLDALE SGRDEQGLPL SYDKAVVAAG YPAHTADLEN | |
| | GERDALPYYG ELLWRYTQDA PTAKNDAERK FGKIANPTVH IGLNQLRKLV | |
| | NALIQRYGKP AQIVVELARN LKAGLEEKER IKKQQTANLE RNERIRQKLQ | |
| | DAGVPDNREN RLRMRLFEEL GQGNGLGTPC IYSGRQISLQ RLFSNDVQVD | |
| | HILPFSKTLD DSFANKVLAQ HDANRYKGNR GPFEAFGANR DGYAWDDIRA | |
| | RAAVLPRNKR NRFAETAMQD WLHNETDFLA RQLTDTAYLS RVARQYLTAI | |
| | CSKDDVYVSP GRLTAMLRAK WGLNRVLDGV MEEQGRPAVK NRDDHRHHAI | |
| | DAVVIGATDR AMLQQVATLA ARAREQDAER LIGDMPTPWP NFLEDVRAAV | |
| | ARCVVSHKPD HGPEGGLHND TAYGIVAGPF EDGRYRVRHR VSLFDLKPGD | |
| | LSNVRCDAPL QAELEPIFEQ DDARAREVAL TALAERYRQR KVWLEELMSV | |
| | LPIRPRGEDG KTLPDSAPYK AYKGDSNYCY ELFINERGRW DGELISTFRA | |
| | NQAAYRRFRN DPARFRRYTA GGRPLLMRLC INDYIAVGTA AERTIFRVVK | |
| | MSENKITLAE HFEGGTLKQR DADKDDPFKY LTKSPGALRD LGARRIFVDL | |
| | IGRVLDPGIK GD | |
| Catenibacterium | IVDYCIGLDL GTGSVGWAVV DMNHRLMKRN GKHLWGSRLF SNAETAANRR | (SEQ |
| mitsuokai | ASRSIRRRYN KRRERIRLLR AILQDMVLEK DPTFFIRLEH TSFLDEEDKA | ID NO: |
| DSM 15897 | KYLGTDYKDN YNLFIDEDFN DYTYYHKYPT IYHLRKALCE STEKADPRLI | 68) |
| WP_ | YLALHHIVKY RGNFLYEGQK FNMDASNIED KLSDIFTQFT SFNNIPYEDD | |
| 006506696.1 | EKKNLEILEI LKKPLSKKAK VDEVMTLIAP EKDYKSAFKE LVTGIAGNKM | |
| | NVTKMILCEP IKQGDSEIKL KFSDSNYDDQ FSEVEKDLGE YVEFVDALHN | |
| | VYSWVELQTI MGATHTDNAS ISEAMVSRYN KHHDDLKLLK DCIKNNVPNK | |
| | YFDMFRNDSE KSKGYYNYIN RPSKAPVDEF YKYVKKCIEK VDTPEAKQIL | |
| | NDIELENFLL KQNSRINGSV PYQMQLDEMI KIIDNQAEYY PILKEKREQL | |
| | LSILTFRIPY YFGPLNETSE HAWIKRLEGK ENQRILPWNY QDIVDVDATA | |
| | EGFIKRMRSY CTYFPDEEVL PKNSLIVSKY EVYNELNKIR VDDKLLEVDV | |
| | KNDIYNELFM KNKTVTEKKL KNWLVNNQCC SKDAEIKGFQ KENQFSTSLT | |
| | PWIDFTNIFG KIDQSNFDLI ENIIYDLTVF EDKKIMKRRL KKKYALPDDK | |
| | VKQILKLKYK DWSRLSKKLL DGIVADNRFG SSVTVLDVLE MSRLNLMEII | |
| | NDKDLGYAQM IEEATSCPED GKFTYEEVER LAGSPALKRG IWQSLQIVEE | |
| | ITKVMKCRPK YIYIEFERSE EAKERTESKI KKLENVYKDL DEQTKKEYKS | |
| | VLEELKGFDN TKKISSDSLF LYFTQLGKCM YSGKKLDIDS LDKYQIDHIV | |
| | PQSLVKDDSF DNRVLVVPSE NQRKLDDLVV PFDIRDKMYR FWKLLFDHEL | |
| | ISPKKFYSLI KTEYTERDEE RFINRQLVET RQITKNVTQI IEDHYSTTKV | |
| | AAIRANLSHE FRVKNHIYKN RDINDYHHAH DAYIVALIGG FMRDRYPNMH | |
| | DSKAVYSEYM KMFRKNKNDQ KRWKDGFVIN SMNYPYEVDG KLIWNPDLIN | |
| | EIKKCFYYKD CYCTTKLDQK SGQLFNLTVL SNDAHADKGV TKAVVPVNKN | |
| | RSDVHKYGGF SGLQYTIVAI EGQKKKGKKT ELVKKISGVP LHLKAASINE | |
| | KINYIEEKEG LSDVRIIKDN IPVNQMIEMD GGEYLLTSPT EYVNARQLVL | |
| | NEKQCALIAD IYNAIYKQDY DNLDDILMIQ LYIELTNKMK VLYPAYRGIA | |
| | EKFESMNENY WVISKEEKAN IIKQMLIVMH RGPQNGNIVY DDFKISDRIG | |
| | RLKTKNHNLN NIVFISQSPT GIYTKKYKL | |
| Mycoplasma | MLRLYCANNL VLNNVQNLWK YLLLLIFDKK IIFLFKIKVI LIRRYMENNN | (SEQ |
| synoviae 53 | KEKIVIGFDL GVASVGWSIV NAETKEVIDL GVRLFSEPEK ADYRRAKRTT | ID NO: |
| AOL40776.1 | RRLLRRKKFK REKFHKLILK NAEIFGLQSR NEILNVYKDQ SSKYRNILKL | 69) |
| | KINALKEEIK PSELVWILRD YLQNRGYFYK NEKLTDEFVS NSFPSKKLHE | |
| | HYEKYGFFRG SVKLDNKLDN KKDKAKEKDE EEESDAKKES EELIFSNKQW | |
| | INEIVKVFEN QSYLTESFKE EYLKLFNYVR PFNKGPGSKN SRTAYGVFST | |
| | DIDPETNKFK DYSNIWDKTI GKCSLFEEEI RAPKNLPSAL IFNLQNEICT | |
| | IKNEFTEFKN WWLNAEQKSE ILKFVFTELF NWKDKKYSDK KFNKNLQDKI | |
| | KKYLLNFALE NFNLNEEILK NRDLENDTVL GLKGVKYYEK SNATADAALE | |
| | FSSLKPLYVF IKFLKEKKLD LNYLLGLENT EILYFLDSIY LAISYSSDLK | |
| | ERNEWFKKLL KELYPKIKNN NLEIIENVED IFEITDQEKF ESFSKTHSLS | |
| | REAFNHIIPL LLSNNEGKNY ESLKHSNEEL KKRTEKAELK AQQNQKYLKD | |
| | NFLKEALVPL SVKTSVLQAI KIFNQIIKNF GKKYEISQVV IEMARELTKP | |
| | NLEKLLNNAT NSNIKILKEK LDQTEKFDDF TKKKFIDKIE NSVVFRNKLF | |
| | LWFEQDRKDP YTQLDIKINE IEDETEIDHV IPYSKSADDS WFNKLLVKKS | |
| | TNQLKKNKTV WEYYQNESDP EAKWNKFVAW AKRIYLVQKS DKESKDNSEK | |
| | NSIFKNKKPN LKFKNITKKL FDPYKDLGFL ARNLNDTRYA TKVFRDQLNN | |
| | YSKHHSKDDE NKLFKVVCMN GSITSFLRKS MWRKNEEQVY RFNFWKKDRD | |
| | QFFHHAVDAS IIAIFSLLTK TLYNKLRVYE SYDVQRREDG VYLINKETGE | |
| | VKKADKDYWK DQHNFLKIRE NAIEIKNVLN NVDFQNQVRY SRKANTKLNT | |
| | QLFNETLYGV KEFENNFYKL EKVNLFSRKD LRKFILEDLN EESEKNKKNE | |
| | NGSRKRILTE KYIVDEILQI LENEEFKDSK SDINALNKYM DSLPSKFSEF | |
| | FSQDFINKCK KENSLILTFD AIKHNDPKKV IKIKNLKFFR EDATLKNKQA | |
| | VHKDSKNQIK SFYESYKCVG FIWLKNKNDL EESIFVPINS RVIHFGDKDK | |
| | DIFDFDSYNK EKLLNEINLK RPENKKFNSI NEIEFVKFVK PGALLLNFEN | |
| | QQIYYISTLE SSSLRAKIKLLNKMDKGKAVS MKKITNPDEY KIIEHVNPL | |
| | GINLNWTKKL ENNN | |
| Flavobacterium | MAKILGLDLG TNSIGWAVVE RENIDFSLID KGVRIFSEGV KSEKGIESSR | (SEQ |
| branchiophilum | AAERTGYRSA RKIKYRRKLR KYETLKVLSL NRMCPLSIEE VEEWKKSGFK | ID NO: |
| FL-15 | DYPLNPEFLK WLSTDEESNV NPYFFRDRAS KHKVSLFELG RAFYHIAQRR | 70) |
| WP_01408415 | GFLSNRLDQS AEGILEEHCP KIEAIVEDLI SIDEISTNIT DYFFETGILD | |
| 1.1 | SNEKNGYAKD LDEGDKKLVS LYKSLLAILK KNESDFENCK SEIIERLNKK | |
| | DVLGKVKGKI KDISQAMLDG NYKTLGQYFY SLYSKEKIRN QYTSREEHYL | |
| | SEFITICKVQ GIDQINEEEK INEKKFDGLA KDLYKAIFFQ RPLKSQKGLI | |
| | GKCSFEKSKS RCAISHPDFE EYRMWTYLNT IKIGTQSDKK LRFLTQDEKL | |
| | KLVPKFYRKN DFNFDVLAKE LIEKGSSFGF YKSSKKNDFF YWFNYKPTDT | |
| | VAACQVAASL KNAIGEDWKT KSFKYQTINS NKEQVSRTVD YKDLWHLLTV | |
| | ATSDVYLYEF AIDKLGLDEK NAKAFSKTKL KKDFASLSLS AINKILPYLK | |
| | EGLLYSHAVF VANIENIVDE NIWKDEKQRD YIKTQISEII ENYTLEKSRF | |
| | EIINGLLKEY KSENEDGKRV YYSKEAEQSF ENDLKKKLVL FYKSNEIENK | |
| | EQQETIFNEL LPIFIQQLKD YEFIKIQRLD QKVLIFLKGK NETGQIFCTE | |
| | EKGTAEEKEK KIKNRLKKLY HPSDIEKFKK KIIKDEFGNE KIVLGSPLTP | |
| | SIKNPMAMRA LHQLRKVLNA LILEGQIDEK TIIHIEMARE LNDANKRKGI | |
| | QDYQNDNKKF REDAIKEIKK LYFEDCKKEV EPTEDDILRY QLWMEQNRSE | |
| | IYEEGKNISI CDIIGSNPAY DIEHTIPRSR SQDNSQMNKT LCSQRFNREV | |
| | KKQSMPIELN NHLEILPRIA HWKEEADNLT REIEIISRSI KAAATKEIKD | |
| | KKIRRRHYLT LKRDYLQGKY DRFIWEEPKV GFKNSQIPDT GIITKYAQAY | |
| | LKSYFKKVES VKGGMVAEFR KIWGIQESFI DENGMKHYKV KDRSKHTHHT | |
| | IDAITIACMT KEKYDVLAHA WTLEDQQNKK EARSIIEASK PWKTFKEDLL | |
| | KIEEEILVSH YTPDNVKKQA KKIVRVRGKK QFVAEVERDV NGKAVPKKAA | |
| | SGKTIYKLDG EGKKLPRLQQ GDTIRGSLHQ DSIYGAIKNP LNTDEIKYVI | |
| | RKDLESIKGS DVESIVDEVV KEKIKEAIAN KVLLLSSNAQ QKNKLVGTVW | |
| | MNEEKRIAIN KVRIYANSVK NPLHIKEHSL LSKSKHVHKQ KVYGQNDENY | |
| | AMAIYELDGK RDFELINIFN LAKLIKQGQG FYPLHKKKEI KGKIVFVPIE | |
| | KRNKRDVVLK RGQQVVFYDK EVENPKDISE IVDFKGRIYI IEGLSIQRIV | |
| | RPSGKVDEYG VIMLRYFKEA RKADDIKQDN FKPDGVFKLG ENKPTRKMNH | |
| | NQFTAFVEGI DFKVLPSGKF EKI | |
| Eubacterium | MENKQYYIGL DVGTNSVGWA VTDTSYNLLR AKGKDMWGAR LFEKANTAAE | (SEQ |
| yurii subsp. | RRTKRTSRRR SEREKARKAM LKELFADEIN RVDPSFFIRL EESKFFLDDR | ID NO: |
| margaretiae | SENNRQRYTL FNDATFTDKD YYEKYKTIFH LRSALINSDE KFDVRLVFLA | 71) |
| ATCC 43715 | ILNLFSHRGH FLNASLKGDG DIQGMDVFYN DLVESCEYFE IELPRITNID | |
| EFM38267.1 | NFEKILSQKG KSRTKILEEL SEELSISKKD KSKYNLIKLI SGLEASVVEL | |
| | YNIEDIQDEN KKIKIGFRES DYEESSLKVK EIIGDEYFDL VERAKSVHDM | |
| | GLLSNIIGNS KYLCEARVEA YENHHKDLLK IKELLKKYDK KAYNDMFRKM | |
| | TDKNYSAYVG SVNSNIAKER RSVDKRKIED LYKYIEDTAL KNIPDDNKDK | |
| | IEILEKIKLG EFLKKQLTAS NGVIPNQLQS RELRAILKKA ENYLPFLKEK | |
| | GEKNLTVSEM IIQLFEFQIP YYVGPLDKNP KKDNKANSWA KIKQGGRILP | |
| | WNFEDKVDVK GSRKEFIEKM VRKCTYISDE HTLPKQSLLY EKFMVLNEIN | |
| | NIKIDGEKIS VEAKQKIYND LFVKGKKVSQ KDIKKELISL NIMDKDSVLS | |
| | GTDTVCNAYL SSIGKFTGVF KEEINKQSIV DMIEDIIFLK TVYGDEKRFV | |
| | KEEIVEKYGD EIDKDKIKRI LGFKFSNWGN LSKSFLELEG ADVGTGEVRS | |
| | IIQSLWETNF NLMELLSSRF TYMDELEKRV KKLEKPLSEW TIEDLDDMYL | |
| | SSPVKRMIWQ SMKIVDEIQT VIGYAPKRIF VEMTRSEGEK VRTKSRKDRL | |
| | KELYNGIKED SKQWVKELDS KDESYFRSKK MYLYYLQKGR CMYSGEVIEL | |
| | DKLMDDNLYD IDHIYPRSFV KDDSLDNLVL VKKEINNRKQ NDPITPQIQA | |
| | SCQGFWKILH DQGFMSNEKY SRLTRKTQEF SDEEKLSFIN RQIVETGQAT | |
| | KCMAQILQKS MGEDVDVVFS KARLVSEFRH KFELFKSRLI NDFHHANDAY | |
| | LNIVVGNSYF VKFTRNPANF IKDARKNPDN PVYKYHMDRF FERDVKSKSE | |
| | VAWIGQSEGN SGTIVIVKKT MAKNSPLITK KVEEGHGSIT KETIVGVKEI | |
| | KFGRNKVEKA DKTPKKPNLQ AYRPIKTSDE RLCNILRYGG RTSISISGYC | |
| | LVEYVKKRKT IRSLEAIPVY LGRKDSLSEE KLLNYFRYNL NDGGKDSVSD | |
| | IRLCLPFIST NSLVKIDGYL YYLGGKNDDR IQLYNAYQLK MKKEEVEYIR | |
| | KIEKAVSMSK FDEIDREKNP VLTEEKNIEL YNKIQDKFEN TVFSKRMSLV | |
| | KYNKKDLSFG DFLKNKKSKF EEIDLEKQCK VLYNIIFNLS NLKEVDLSDI | |
| | GGSKSTGKCR CKKNITNYKE FKLIQQSITG LYSCEKDLMT I | |
| Acidovorax | MAQHVFGLDI GIASVGWAIL GEQRIIDLGV RCFDKAETAK EGDPLNLTRR | (SEQ |
| ebreus | QARLLRRRLY RRAWRLTQLS RLLKRKGLIA DAKLFAKAPS YGDSAWELRR | ID NO: |
| WP_01265517 | QGLDRLLTPL EWARVIYHQC KHRGFHWTSK AEEAKADSDA EGGRVKQGLA | 72) |
| 6.1 | HTKALMQAKN YRSAAEMVLA EFPDAQRNKR GQYDKALSRV LLGEELALLF | |
| | ATQRRLGNPH ASDFFEKLIL GDGDRKSGLF WQQKPALSGA DLLKMLGKCT | |
| | FEKGEYRAPK ASFSVERHVW LTRLNNLRIV VDGRSRPLNE AERQAALLLP | |
| | YQTETSKYKT LKNAFIKAGL WGDGVRFGGL AYPSQAQIDA EKTKDPEDQF | |
| | LVKLPAWHEL RKAFKAAGHE ALWQQISTPA LDGDPTLLDQ IATVLSVYKD | |
| | GAEVVQQLRQ LALPEPAASI AVLEKISFDK FSSLSLKALR RIVPLMQSGL | |
| | RYDEAVAQIP EYGHHSQRIE PGAAKHLYLP PFYEAQRKYA GKGDHIGSMQ | |
| | FRDDADIPRN PWVLRALNQA RKVVNALIRE YGSPIAVNIE MARDLSRPLD | |
| | ERNKVKRAQE EFRDRNDRAR SEFERDFGYK PKAAAFEKWM LYREQLGQCA | |
| | YSQQPLDIQR VLDDHNYAQV DHALPYSRSY DDSKNNKVLV LTHENQNKGN | |
| | RTAFEYLTSF PDGEDGERWR TFVAWVQGNK AYRMAKRNRL LRKNYGVDES | |
| | KGFIDRNLND TRYICKFFKN YVEEHLQLAA RADGDTARRC VVVNGQLTAF | |
| | LRARWGLTKV RGDSDRHHAL DAAVVAACTH GMVKALADYS RRKEISFLQE | |
| | GFPDPETGEI LNPAAFDRAR QHFPEPWTHF AHELKARLFT DDLAALREDM | |
| | QRLGSYTTED LGRLRTLFVS RAPQRRSGGA VHKETIYAQP ESLKQQGGVI | |
| | EKILLTSLKL QDFDKLLNPE SNDHFVEPHR NERLYAAIRQ RLEQFGGRAD | |
| | KAFGPDNLFH KPDKNNQPTG PVVRSIKLVR GKQTGIPIRG GLAKNDSMLR | |
| | VDIFTKAGKF HLVPVYVHHR VTGLPNRAIV AFKDEDEWTL IDESFAFLFS | |
| | VYPNDYVKVT LKKEQQSGYY SGADRSTGAM NLWAHDRAAS VGKDGLIRGI | |
| | GVKTALSVEK FNVDVLGRIY LAPPETRSGL A | |
| Porphyromonas | MLMSKHVLGL DLGVGSIGWC LIALDAQGDP AEILGMGSRV VPLNNATKAI | (SEQ |
| sp. oral taxon | EAFNAGAAFT ASQERTARRT MRRGFARYQL RRYRLRRELE KVGMLPDAAL | ID NO: |
| 279 str. F0450 | IQLPLLELWE LRERAATAGR RLTLPELGRV LCHINQKRGY RHVKSDAAAI | 73) |
| WP_00943351 | VGDEGEKKKD SNSAYLAGIR ANDEKLQAEH KTVGQYFAEQ LRQNQSESPT | |
| 8.1 | GGISYRIKDQ IFSRQCYIDE YDQIMAVQRV HYPDILTDEF IRMLRDEVIF | |
| | MQRPLKSCKH LVSLCEFEKQ ERVMRVQQDD GKGGWQLVER RVKFGPKVAP | |
| | KSSPLFQLCC IYEAVNNIRL TRPNGSPCDI TPEERAKIVA HLQSSASLSF | |
| | AALKKLLKEK ALIADQLTSK SGLKGNSTRV ALASALQPYP QYHHLLDMEL | |
| | ETRMMTVQLT DEETGEVTER EVAWTDSYV RKPLYRLWHI LYSIEEREAM | |
| | RRALITQLGM KEEDLDGGLL DQLYRLDFVK PGYGNKSAKF ICKLLPQLQQ | |
| | GLGYSEACAA VGYRHSNSPT SEEITERTLL EKIPLLQRNE LRQPLVEKIL | |
| | NQMINLVNAL KAEYGIDEVR VELARELKMS REERERMARN NKDREERNKG | |
| | VAAKIRECGL YPTKPRIQKY MLWKEAGRQC LYCGRSIEEE QCLREGGMEV | |
| | EHIIPKSVLY DDSYGNKTCA CRRCNKEKGN RTALEYIRAK GREAEYMKRI | |
| | NDLLKEKKIS YSKHQRLRWL KEDIPSDFLE RQLRLTQYIS RQAMAILQQG | |
| | IRRVSASEGG VTARLRSLWG YGKILHTLNL DRYDSMGETE RVSREGEATE | |
| | ELHITNWSKR MDHRHHAIDA LVVACTRQSY IQRLNRLSSE FGREDKKKED | |
| | QEAQEQQATE TGRLSNLERW LTQRPHFSVR TVSDKVAEIL ISYRPGQRVV | |
| | TRGRNIYRKK MADGREVSCV QRGVLVPRGE LMEASFYGKI LSQGRVRIVK | |
| | RYPLHDLKGE VVDPHLRELI TTYNQELKSR EKGAPIPPLC LDKDKKQEVR | |
| | SVRCYAKTLS LDKAIPMCFD EKGEPTAFVK SASNHHLALY RTPKGKLVES | |
| | IVTFWDAVDR ARYGIPLVIT HPREVMEQVL QRGDIPEQVL SLLPPSDWVF | |
| | VDSLQQDEMV VIGLSDEELQ RALEAQNYRK ISEHLYRVQK MSSSYYVFRY | |
| | HLETSVADDK NTSGRIPKFH RVQSLKAYEE RNIRKVRVDL LGRISLL | |
| Mycoplasma | MHNKKNITIG FDLGIASIGW AIIDSTTSKI LDWGTRTFEE RKTANERRAF | (SEQ |
| ovipneumoniae | RSTRRNIRRK AYRNQRFINL ILKYKDLFEL KNISDIQRAN KKDTENYEKI | ID NO: |
| SC01 | ISFFTEIYKK CAAKHSNILE VKVKALDSKI EKLDLIWILH DYLENRGFFY | 74) |
| WP_01032092 | DLEEENVADK YEGIEHPSIL LYDFFKKNGF FKSNSSIPKD LGGYSFSNLQ | |
| 2.1 | WVNEIKKLFE VQEINPEFSE KFLNLFTSVR DYAKGPGSEH SASEYGIFQK | |
| | DEKGKVFKKY DNIWDKTIGK CSFFVEENRS PVNYPSYEIF NLLNQLINLS | |
| | TDLKTTNKKI WQLSSNDRNE LLDELLKVKE KAKIISISLK KNEIKKIILK | |
| | DFGFEKSDID DQDTIEGRKI IKEEPTTKLE VTKHLLATIY SHSSDSNWIN | |
| | INNILEFLPY LDAICIILDR EKSRGQDEVL KKLTEKNIFE VLKIDREKQL | |
| | DFVKSIFSNT KFNFKKIGNF SLKAIREFLP KMFEQNKNSE YLKWKDEEIR | |
| | RKWEEQKSKL GKTDKKTKYL NPRIFQDEII SPGTKNTFEQ AVLVLNQIIK | |
| | KYSKENIIDA IIIESPREKN DKKTIEEIKK RNKKGKGKTL EKLFQILNLE | |
| | NKGYKLSDLE TKPAKLLDRL RFYHQQDGID LYTLDKINID QLINGSQKYE | |
| | IEHIIPYSMS YDNSQANKIL TEKAENLKKG KLIASEYIKR NGDEFYNKYY | |
| | EKAKELFINK YKKNKKLDSY VDLDEDSAKN RFRFLTLQDY DEFQVEFLAR | |
| | NLNDTRYSTK LFYHALVEHF ENNEFFTYID ENSSKHKVKI STIKGHVTKY | |
| | FRAKPVQKNN GPNENLNNNK PEKIEKNREN NEHHAVDAAI VAIIGNKNPQ | |
| | IANLLTLADN KTDKKFLLHD ENYKENIETG ELVKIPKFEV DKLAKVEDLK | |
| | KIIQEKYEEA KKHTAIKFSR KTRTILNGGL SDETLYGFKY DEKEDKYFKI | |
| | IKKKLVTSKN EELKKYFENP FGKKADGKSE YTVLMAQSHL SEFNKLKEIF | |
| | EKYNGFSNKT GNAFVEYMND LALKEPTLKA EIESAKSVEK LLYYNFKPSD | |
| | QFTYHDNINN KSFKRFYKNI RIIEYKSIPI KFKILSKHDG GKSFKDTLFS | |
| | LYSLVYKVYE NGKESYKSIP VTSQMRNFGI DEFDFLDENL YNKEKLDIYK | |
| | SDFAKPIPVN CKPVFVLKKG SILKKKSLDI DDFKETKETE EGNYYFISTI | |
| | SKRFNRDTAY GLKPLKLSVV KPVAEPSTNP IFKEYIPIHL DELGNEYPVK | |
| | IKEHTDDEKL MCTIK | |
| Wolinella | MLVSPISVDL GGKNTGFFSF TDSLDNSQSG TVIYDESFVL SQVGRRSKRH | (SEQ |
| succinogenes | SKRNNLRNKL VKRLFLLILQ EHHGLSIDVL PDEIRGLFNK RGYTYAGFEL | ID NO: |
| WP_01113943 | DEKKKDALES DTLKEFLSEK LQSIDRDSDV EDFLNQIASN AESFKDYKKG | 75) |
| 1.1 | FEAVFASATH SPNKKLELKD ELKSEYGENA KELLAGLRVT KEILDEFDKQ | |
| | ENQGNLPRAK YFEELGEYIA TNEKVKSFFD SNSLKLTDMT KLIGNISNYQ | |
| | LKELRRYFND KEMEKGDIWI PNKLHKITER FVRSWHPKND ADRQRRAELM | |
| | KDLKSKEIME LLTTTEPVMT IPPYDDMNNR GAVKCQTLRL NEEYLDKHLP | |
| | NWRDIAKRLN HGKFNDDLAD STVKGYSEDS TLLHRLLDTS KEIDIYELRG | |
| | KKPNELLVKT LGQSDANRLY GFAQNYYELI RQKVRAGIWV PVKNKDDSLN | |
| | LEDNSNMLKR CNHNPPHKKN QIHNLVAGIL GVKLDEAKFA EFEKELWSAK | |
| | VGNKKLSAYC KNIEELRKTH GNTFKIDIEE LRKKDPAELS KEEKAKLRLT | |
| | DDVILNEWSQ KIANFFDIDD KHRQRFNNLF SMAQLHTVID TPRSGFSSTC | |
| | KRCTAENRFR SETAFYNDET GEFHKKATAT CQRLPADTQR PFSGKIERYI | |
| | DKLGYELAKI KAKELEGMEA KEIKVPIILE QNAFEYEESL RKSKTGSNDR | |
| | VINSKKDRDG KKLAKAKENA EDRLKDKDKR IKAFSSGICP YCGDTIGDDG | |
| | EIDHILPRSH TLKIYGTVFN PEGNLIYVHQ KCNQAKADSI YKLSDIKAGV | |
| | SAQWIEEQVA NIKGYKTFSV LSAEQQKAFR YALFLQNDNE AYKKVVDWLR | |
| | TDQSARVNGT QKYLAKKIQE KLTKMLPNKH LSFEFILADA TEVSELRRQY | |
| | ARQNPLLAKA EKQAPSSHAI DAVMAFVARY QKVFKDGTPP NADEVAKLAM | |
| | LDSWNPASNE PLTKGLSTNQ KIEKMIKSGD YGQKNMREVF GKSIFGENAI | |
| | GERYKPIVVQ EGGYYIGYPA TVKKGYELKN CKVVTSKNDI AKLEKIIKNQ | |
| | DLISLKENQY IKIFSINKQT ISELSNRYFN MNYKNLVERD KEIVGLLEFI | |
| | VENCRYYTKK VDVKFAPKYI HETKYPFYDD WRRFDEAWRY LQENQNKTSS | |
| | KDRFVIDKSS LNEYYQPDKN EYKLDVDTQP IWDDFCRWYF LDRYKTANDK | |
| | KSIRIKARKT FSLLAESGVQ GKVFRAKRKI PTGYAYQALP MDNNVIAGDY | |
| | ANILLEANSK TLSLVPKSGI SIEKQLDKKL DVIKKTDVRG LAIDNNSFFN | |
| | ADFDTHGIRL IVENTSVKVG NFPISAIDKS AKRMIFRALF EKEKGKRKKK | |
| | TTISFKESGP VQDYLKVFLK KIVKIQLRTD GSISNIWRK NAADFTLSFR | |
| | SEHIQKLLK | |
| Streptococcus | MKKPYSIGLD IGTNSVGWAV VTDDYKVPAK KMKVLGNTDK SHIEKNLLGA | (SEQ |
| mutans UA159 | LLFDSGNTAE DRRLKRTARR RYTRRRNRIL YLQEIFSEEM GKVDDSFFHR | ID NO: |
| WP_00226354 | LEDSFLVTED KRGERHPIFG NLEEEVKYHE NFPTIYHLRQ YLADNPEKVD | 76) |
| 9.1 | LRLVYLALAH IIKFRGHFLI EGKFDTRNND VQRLFQEFLA VYDNTFENSS | |
| | LQEQNVQVEE ILTDKISKSA KKDRVLKLFP NEKSNGRFAE FLKLIVGNQA | |
| | DFKKHFELEE KAPLQFSKDT YEEELEVLLA QIGDNYAELF LSAKKLYDSI | |
| | LLSGILTVTD VGTKAPLSAS MIQRYNEHQM DLAQLKQFIR QKLSDKYNEV | |
| | FSDVSKDGYA GYIDGKTNQE AFYKYLKGLL NKIEGSGYFL DKIEREDFLR | |
| | KQRTFDNGSI PHQIHLQEMR AIIRRQAEFY PFLADNQDRI EKLLTFRIPY | |
| | YVGPLARGKS DFAWLSRKSA DKITPWNFDE IVDKESSAEA FINRMTNYDL | |
| | YLPNQKVLPK HSLLYEKFTV YNELTKVKYK TEQGKTAFFD ANMKQEIFDG | |
| | VFKVYRKVTK DKLMDFLEKE FDEFRIVDLT GLDKENKVEN ASYGTYHDLC | |
| | KILDKDFLDN SKNEKILEDI VLTLTLFEDR EMIRKRLENY SDLLTKEQVK | |
| | KLERRHYTGW GRLSAELIHG IRNKESRKTI LDYLIDDGNS NRNFMQLIND | |
| | DALSFKEEIA KAQVIGETDN LNQVVSDIAG SPAIKKGILQ SLKIVDELVK | |
| | IMGHQPENIV VEMARENQFT NQGRRNSQQR LKGLTDSIKE FGSQILKEHP | |
| | VENSQLQNDR LFLYYLQNGR DMYTGEELDI DYLSQYDIDH IIPQAFIKDN | |
| | SIDNRVLTSS KENRGKSDDV PSKDVVRKMK SYWSKLLSAK LITQRKFDNL | |
| | TKAERGGLTD DDKAGFIKRQ LVETRQITKH VARILDERFN TETDENNKKI | |
| | RQVKIVTLKS NLVSNFRKEF ELYKVREIND YHHAHDAYLN AVIGKALLGV | |
| | YPQLEPEFVY GDYPHFHGHK ENKATAKKFF YSNIMNFFKK DDVRTDKNGE | |
| | IIWKKDEHIS NIKKVLSYPQ VNIVKKVEEQ TGGFSKESIL PKGNSDKLIP | |
| | RKTKKFYWDT KKYGGFDSPI VAYSILVIAD IEKGKSKKLK TVKALVGVTI | |
| | MEKMTFERDP VAFLERKGYR NVQEENIIKL PKYSLFKLEN GRKRLLASAR | |
| | ELQKGNEIVL PNHLGTLLYH AKNIHKVDEP KHLDYVDKHK DEFKELLDVV | |
| | SNFSKKYTLA EGNLEKIKEL YAQNNGEDLK ELASSFINLL TFTAIGAPAT | |
| | FKFFDKNIDR KRYTSTTEIL NATLIHQSIT GLYETRIDLN KLGGD | |
| Prevotella | MNKRILGLDT GTNSLGWAVV DWDEHAQSYE LIKYGDVIFQ EGVKIEKGIE | (SEQ |
| timonensis | SSKAAERSGY KAIRKQYFRR RLRKIQVLKV LVKYHLCPYL SDDDLRQWHL | ID NO: |
| CRIS 5C-B1 | QKQYPKSDEL MLWQRTSDEE GKNPYYDRHR CLHEKLDLTV EADRYTLGRA | |
| WP_00812271 | LYHLTQRRGF LSNRLDTSAD NKEDGVVKSG ISQLSTEMEE AGCEYLGDYF | |
| 8.1 | YKLYDAQGNK VRIRQRYTDR NKHYQHEFDA ICEKQELSSE LIEDLQRAIF | |
| | FQLPLKSQRH GVGRCTFERG KPRCADSHPD YEEFRMLCFV NNIQVKGPHD | 77) |
| | LELRPLTYEE REKIEPLFFR KSKPNFDFED IAKALAGKKN YAWIHDKEER | |
| | AYKFNYRMTQ GVPGCPTIAQ LKSIFGDDWK TGIAETYTLI QKKNGSKSLQ | |
| | EMVDDVWNVL YSFSSVEKLK EFAHHKLQLD EESAEKFAKI KLSHSFAALS | |
| | LKAIRKFLPF LRKGMYYTHA SFFANIPTIV GKEIWNKEQN RKYIMENVGE | |
| | LVFNYQPKHR EVQGTIEMLI KDFLANNFEL PAGATDKLYH PSMIETYPNA | |
| | QRNEFGILQL GSPRTNAIRN PMAMRSLHIL RRVVNQLLKE SIIDENTEVH | |
| | VEYARELNDA NKRRAIADRQ KEQDKQHKKY GDEIRKLYKE ETGKDIEPTQ | |
| | TDVLKFQLWE EQNHHCLYTG EQIGITDFIG SNPKFDIEHT IPQSVGGDST | |
| | QMNLTLCDNR FNREVKKAKL PTELANHEEI LTRIEPWKNK YEQLVKERDK | |
| | QRTFAGMDKA VKDIRIQKRH KLQMEIDYWR GKYERFTMTE VPEGFSRRQG | |
| | TGIGLISRYA GLYLKSLFHQ ADSRNKSNVY VVKGVATAEF RKMWGLQSEY | |
| | EKKQRDNHSH HCMDAITIAC IGKREYDLMA EYYRMEETFK QGRGSKPKFS | |
| | KPWATFTEDV LNIYKNLLVV HDTPNNMPKH TKKYVQTSIG KVLAQGDTAR | |
| | GSLHLDTYYG AIERDGEIRY VVRRPLSSFT KPEELENIVD ETVKRTIKEA | |
| | IADKNFKQAI AEPIYMNEEK GILIKKVRCF AKSVKQPINI RQHRDLSKKE | |
| | YKQQYHVMNE NNYLLAIYEG LVKNKVVREF EIVSYIEAAK YYKRSQDRNI | |
| | FSSIVPTHST KYGLPLKTKL LMGQLVLMFE ENPDEIQVDN TKDLVKRLYK | |
| | WGIEKDGRI KFKYHQEARK EGLPIFSTPY KNNDDYAPIF RQSINNINIL | |
| | VDGIDFTIDI LGKVTLKE | |
| Clostridium | MKYTLGLDVG IASVGWAVID KDNNKIIDLG VRCFDKAEES KTGESLATAR | (SEQ |
| cellulolyticum | RIARGMRRRI SRRSQRLRLV KKLFVQYEII KDSSEFNRIF DTSRDGWKDP | ID NO: |
| H10 | WELRYNALSR ILKPYELVQV LTHITKRRGF KSNRKEDLST TKEGWVITSI | 78) |
| ACL77411.1 | KNNSEMLRTK NYRTIGEMIF METPENSNKR NKVDEYIHTI AREDLLNEIK | |
| | YIFSIQRKLG SPFVTEKLEH DFLNIWEFQR PFASGDSILS KVGKCTLLKE | |
| | ELRAPTSCYT SEYFGLLQSI NNLVLVEDNN TLTLNNDQRA KIIEYAHFKN | |
| | EIKYSEIRKL LDIEPEILFK AHNLTHKNPS GNNESKKFYE MKSYHKLKST | |
| | LPTDIWGKLH SNKESLDNLF YCLTVYKNDN EIKDYLQANN LDYLIEYIAK | |
| | LPTFNKFKHL SLVAMKRIIP FMEKGYKYSD ACNMAELDFT GSSKLEKCNK | |
| | LTVEPIIENV TNPVVIRALT QARKVINAII QKYGLPYMVN IELAREAGMT | |
| | RQDRDNLKKE HENNRKAREK ISDLIRQNGR VASGLDILKW RLWEDQGGRC | |
| | AYSGKPIPVC DLLNDSLTQI DHIYPYSRSM DDSYMNKVLV LTDENQNKRS | |
| | YTPYEVWGST EKWEDFEARI YSMHLPQSKE KRLLNRNFIT KDLDSFISRN | |
| | LNDTRYISRF LKNYIESYLQ FSNDSPKSCV VCVNGQCTAQ LRSRWGLNKN | |
| | REESDLHHAL DAAVIACADR KIIKEITNYY NERENHNYKV KYPLPWHSFR | |
| | QDLMETLAGV FISRAPRRKI TGPAHDETIR SPKHFNKGLT SVKIPLTTVT | |
| | LEKLETMVKN TKGGISDKAV YNVLKNRLIE HNNKPLKAFA EKIYKPLKNG | |
| | TNGAIIRSIR VETPSYTGVF RNEGKGISDN SLMVRVDVFK KKDKYYLVPI | |
| | YVAHMIKKEL PSKAIVPLKP ESQWELIDST HEFLFSLYQN DYLVIKTKKG | |
| | ITEGYYRSCH RGTGSLSLMP HFANNKNVKI DIGVRTAISI EKYNVDILGN | |
| | KSIVKGEPRR GMEKYNSFKS N | |
| Francisella | MNFKILPIAI DLGVKNTGVF SAFYQKGTSL ERLDNKNGKV YELSKDSYTL | (SEQ |
| tularensis | LMNNRTARRH QRRGIDRKQL VKRLFKLIWT EQLNLEWDKD TQQAISFLFN | ID NO: |
| subsp. | RRGFSFITDG YSPEYLNIVP EQVKAILMDI FDDYNGEDDL DSYLKLATEQ | 79) |
| novicida U112 | ESKISEIYNK LMQKILEFKL MKLCTDIKDD KVSTKTLKEI TSYEFELLAD | |
| WP_00303894 | YLANYSESLK TQKFSYTDKQ GNLKELSYYH HDKYNIQEFL KRHATINDRI | |
| 1.1 | LDTLLTDDLD IWNFNFEKFD FDKNEEKLQN QEDKDHIQAH LHHFVFAVNK | |
| | IKSEMASGGR HRSQYFQEIT NVLDENNHQE GYLKNFCENL HNKKYSNLSV | |
| | KNLVNLIGNL SNLELKPLRK YFNDKIHAKA DHWDEQKFTE TYCHWILGEW | |
| | RVGVKDQDKK DGAKYSYKDL CNELKQKVTK AGLVDFLLEL DPCRTIPPYL | |
| | DNNNRKPPKC QSLILNPKFL DNQYPNWQQY LQELKKLQSI QNYLDSFETD | |
| | LKVLKSSKDQ PYFVEYKSSN QQIASGQRDY KDLDARILQF IFDRVKASDE | |
| | LLLNEIYFQA KKLKQKASSE LEKLESSKKL DEVIANSQLS QILKSQHTNG | |
| | IFEQGTFLHL VCKYYKQRQR ARDSRLYIMP EYRYDKKLHK YNNTGRFDDD | |
| | NQLLTYCNHK PRQKRYQLLN DLAGVLQVSP NFLKDKIGSD DDLFISKWLV | |
| | EHIRGFKKAC EDSLKIQKDN RGLLNHKINI ARNTKGKCEK EIFNLICKIE | |
| | GSEDKKGNYK HGLAYELGVL LFGEPNEASK PEFDRKIKKF NSIYSFAQIQ | |
| | QIAFAERKGN ANTCAVCSAD NAHRMQQIKI TEPVEDNKDK IILSAKAQRL | |
| | PAIPTRIVDG AVKKMATILA KNIVDDNWQN IKQVLSAKHQ LHIPIITESN | |
| | AFEFEPALAD VKGKSLKDRR KKALERISPE NIFKDKNNRI KEFAKGISAY | |
| | SGANLTDGDF DGAKEELDHI IPRSHKKYGT LNDEANLICV TRGDNKNKGN | |
| | RIFCLRDLAD NYKLKQFETT DDLEIEKKIA DTIWDANKKD FKFGNYRSFI | |
| | NLTPQEQKAF RHALFLADEN PIKQAVIRAI NNRNRTFVNG TQRYFAEVLA | |
| | NNIYLRAKKE NLNTDKISFD YFGIPTIGNG RGIAEIRQLY EKVDSDIQAY | |
| | AKGDKPQASY SHLIDAMLAF CIAADEHRND GSIGLEIDKN YSLYPLDKNT | |
| | GEVFTKDIFS QIKITDNEFS DKKLVRKKAI EGFNTHRQMT RDGIYAENYL | |
| | PILIHKELNE VRKGYTWKNS EEIKIFKGKK YDIQQLNNLV YCLKFVDKPI | |
| | SIDIQISTLE ELRNILTTNN IAATAEYYYI NLKTQKLHEY YIENYNTALG | |
| | YKKYSKEMEF LRSLAYRSER VKIKSIDDVK QVLDKDSNFI IGKITLPFKK | |
| | EWQRLYREWQ NTTIKDDYEF LKSFFNVKSI TKLHKKVRKD FSLPISTNEG | |
| | KFLVKRKTWD NNFIYQILND SDSRADGTKP FIPAFDISKN EIVEAIIDSF | |
| | TSKNIFWLPK NIELQKVDNK NIFAIDTSKW FEVETPSDLR DIGIATIQYK | |
| | IDNNSRPKVR VKLDYVIDDD SKINYFMNHS LLKSRYPDKV LEILKQSTII | |
| | EFESSGFNKT IKEMLGMKLA GIYNETSNN | |
| Azospirillum | MARPAFRAPR REHVNGWTPD PHRISKPFFI LVSWHLLSRV VIDSSSGCFP | (SEQ |
| sp. B510 | GTSRDHTDKF AEWECAVQPY RLSFDLGTNS IGWGLLNLDR QGKPREIRAL | ID NO: |
| AOL40891.1 | GSRIFSDGRD PQDKASLAVA RRLARQMRRR RDRYLTRRTR LMGALVRFGL | 80) |
| | MPADPAARKR LEVAVDPYLA RERATRERLE PFEIGRALFH LNQRRGYKPV | |
| | RTATKPDEEA GKVKEAVERL EAAIAAAGAP TLGAWFAWRK TRGETLRARL | |
| | AGKGKEAAYP FYPARRMLEA EFDTLWAEQA RHHPDLLTAE AREILRHRIF | |
| | HQRPLKPPPV GRCTLYPDDG RAPRALPSAQ RLRLFQELAS LRVIHLDLSE | |
| | RPLTPAERDR IVAFVQGRPP KAGRKPGKVQ KSVPFEKLRG LLELPPGTGF | |
| | SLESDKRPEL LGDETGARIA PAFGPGWTAL PLEEQDALVE LLLTEAEPER | |
| | AIAALTARWA LDEATAAKLA GATLPDFHGR YGRRAVAELL PVLERETRGD | |
| | PDGRVRPIRL DEAVKLLRGG KDHSDFSREG ALLDALPYYG AVLERHVAFG | |
| | TGNPADPEEK RVGRVANPTV HIALNQLRHL VNAILARHGR PEEIVIELAR | |
| | DLKRSAEDRR REDKRQADNQ KRNEERKRLI LSLGERPTPR NLLKLRLWEE | |
| | QGPVENRRCP YSGETISMRM LLSEQVDIDH ILPFSVSLDD SAANKVVCLR | |
| | EANRIKRNRS PWEAFGHDSE RWAGILARAE ALPKNKRWRF APDALEKLEG | |
| | EGGLRARHLN DTRHLSRLAV EYLRCVCPKV RVSPGRLTAL LRRRWGIDAI | |
| | LAEADGPPPE VPAETLDPSP AEKNRADHRH HALDAVVIGC IDRSMVQRVQ | |
| | LAAASAEREA AAREDNIRRV LEGFKEEPWD GFRAELERRA RTIVVSHRPE | |
| | HGIGGALHKE TAYGPVDPPE EGFNLVVRKP IDGLSKDEIN SVRDPRLRRA | |
| | LIDRLAIRRR DANDPATALA KAAEDLAAQP ASRGIRRVRV LKKESNPIRV | |
| | EHGGNPSGPR SGGPFHKLLL AGEVHHVDVA LRADGRRWVG HWVTLFEAHG | |
| | GRGADGAAAP PRLGDGERFL MRLHKGDCLK LEHKGRVRVM QVVKLEPSSN | |
| | SVVVVEPHQV KTDRSKHVKI SCDQLRARGA RRVTVDPLGR VRVHAPGARV | |
| | GIGGDAGRTA MEPAEDIS | |
| Peptoniphilus | MKNLKEYYIG LDIGTASVGW AVTDESYNIP KFNGKKMWGV RLFDDAKTAE | (SEQ |
| duerdenii | ERRTQRGSRR RLNRRKERIN LLQDLFATEI SKVDPNFFLR LDNSDLYRED | ID NO: |
| ATCC BAA- | KDEKLKSKYT LFNDKDFKDR DYHKKYPTIH HLIMDLIEDE GKKDIRLLYL | 81) |
| 1640 | ACHYLLKNRG HFIFEGQKFD TKNSFDKSIN DLKIHLRDEY NIDLEFNNED | |
| WP_008901059.1 | LIEIITDTTL NKTNKKKELK NIVGDTKFLK AISAIMIGSS QKLVDLFEDG | |
| | EFEETTVKSV DFSTTAFDDK YSEYEEALGD TISLLNILKS IYDSSILENL | |
| | LKDADKSKDG NKYISKAFVK KFNKHGKDLK TLKRIIKKYL PSEYANIFRN | |
| | KSINDNYVAY TKSNITSNKR TKASKFTKQE DFYKFIKKHL DTIKETKLNS | |
| | SENEDLKLID EMLTDIEFKT FIPKLKSSDN GVIPYQLKLM ELKKILDNQS | |
| | KYYDFLNESD EYGTVKDKVE SIMEFRIPYY VGPLNPDSKY AWIKRENTKI | |
| | TPWNFKDIVD LDSSREEFID RLIGRCTYLK EEKVLPKASL IYNEFMVLNE | |
| | LNNLKLNEFL ITEEMKKAIF EELFKTKKKV TLKAVSNLLK KEFNLTGDIL | |
| | LSGTDGDFKQ GLNSYIDFKN IIGDKVDRDD YRIKIEEIIK LIVLYEDDKT | |
| | YLKKKIKSAY KNDFTDDEIK KIAALNYKDW GRLSKRFLTG IEGVDKTTGE | |
| | KGSIIYFMRE YNLNLMELMS GHYTFTEEVE KLNPVENREL CYEMVDELYL | |
| | SPSVKRMLWQ SLRVVDEIKR IIGKDPKKIF IEMARAKEAK NSRKESRKNK | |
| | LLEFYKFGKK AFINEIGEER YNYLLNEINS EEESKFRWDN LYLYYTQLGR | |
| | CMYSLEPIDL ADLKSNNIYD QDHIYPKSKI YDDSLENRVL VKKNLNHEKG | |
| | NQYPIPEKVL NKNAYGFWKI LFDKGLIGQK KYTRLTRRTP FEERELAEFI | |
| | ERQIVETRQA TKETANLLKN ICQDSEIVYS KAENASRFRQ EFDIIKCRTV | |
| | NDLHHMHDAY LNIVVGNVYN TKFTKNPLNF IKDKDNVRSY NLENMFKYDV | |
| | VRGSYTAWIA DDSEGNVKAA TIKKVKRELE GKNYRFTRMS YIGTGGLYDQ | |
| | NLMRKGKGQI PQKENTNKSN IEKYGGYNKA SSAYFALIES DGKAGRERTL | |
| | ETIPIMVYNQ EKYGNTEAVD KYLKDNLELQ DPKILKDKIK INSLIKLDGF | |
| | LYNIKGKTGD SLSIAGSVQL IVNKEEQKLI KKMDKFLVKK KDNKDIKVTS | |
| | FDNIKEEELI KLYKTLSDKL NNGIYSNKRN NQAKNISEAL DKFKEISIEE | |
| | KIDVLNQIIL LFQSYNNGQN LKSIGLSAKT GWVFIPKKLN YKECKLINQS | |
| | ITGLFENEVD LLNL | |
| Lactobacillus | MGYRIGLDVG ITSTGYAVLK TDKNGLPYKI LTLDSVIYPR AENPQTGASL | (SEQ |
| coryniformis | AEPRRIKRGL RRRTRRTKFR KQRTQQLFIH SGLLSKPEIE QILATPQAKY | ID NO: |
| subsp. | SVYELRVAGL DRRLTNSELF RVLYFFIGHR GFKSNRKAEL NPENEADKKQ | 82) |
| torquens | MGQLLNSIEE IRKAIAEKGY RTVGELYLKD PKYNDHKRNK GYIDGYLSTP | |
| KCTC 3535 | NRQMLVDEIK QILDKQRELG NEKLTDEFYA TYLLGDENRA GIFQAQRDFD | |
| WP_010014406.1 | EGPGAGPYAG DQIKKMVGKD IFEPTEDRAA KATYTFQYFN LLQKMTSLNY | |
| | QNTTGDTWHT LNGLDRQAII DAVFAKAEKP TKTYKPTDFG ELRKLLKLPD | |
| | DARFNLVNYG SLQTQKEIET VEKKTRFVDF KAYHDLVKVL PEEMWQSRQL | |
| | LDHIGTALTL YSSDKRRRRY FAEELNLPAE LIEKLLPLNF SKFGHLSIKS | |
| | MQNIIPYLEM GQVYSEATTN TGYDFRKKQI SKDTIREEIT NPVVRRAVTK | |
| | TIKIVEQIIR RYGKPDGINI ELARELGRNF KERGDIQKRQ DKNRQTNDKI | |
| | AAELTELGIP VNGQNIIRYK LHKEQNGVDP YTGDQIPFER AFSEGYEVDH | |
| | IIPYSISWDD SYTNKVLTSA KCNREKGNRI PMVYLANNEQ RLNALTNIAD | |
| | NIIRNSRKRQ KLLKQKLSDE ELKDWKQRNI NDTRFITRVL YNYFRQAIEF | |
| | NPELEKKQRV LPLNGEVTSK IRSRWGFLKV REDGDLHHAI DATVIAAITP | |
| | KFIQQVTKYS QHQEVKNNQA LWHDAEIKDA EYAAEAQRMD ADLFNKIFNG | |
| | FPLPWPEFLD ELLARISDNP VEMMKSRSWN TYTPIEIAKL KPVFWVRLAN | |
| | HKISGPAHLD TIRSAKLFDE KGIVLSRVSI TKLKINKKGQ VATGDGIYDP | |
| | ENSNNGDKVV YSAIRQALEA HNGSGELAFP DGYLEYVDHG TKKLVRKVRV | |
| | AKKVSLPVRL KNKAAADNGS MVRIDVFNTG KKFVFVPIYI KDTVEQVLPN | |
| | KAIARGKSLW YQITESDQFC FSLYPGDMVH IESKTGIKPK YSNKENNTSV | |
| | VPIKNFYGYF DGADIATASI LVRAHDSSYT ARSIGIAGLL KFEKYQVDYF | |
| | GRYHKVHEKK RQLFVKRDE | |
| Ignavibacterium | MEFKKVLGLD IGTNSIGCAL LSLPKSIQDY GKGGRLEWLT SRVIPLDADY | (SEQ |
| album JCM | MKAFIDGKNG LPQVITPAGK RRQKRGSRRL KHRYKLRRSR LIRVFKTLNW | ID NO: |
| 16511 | LPEDFPLDNP KRIKETISTE GKFSFRISDY VPISDESYRE FYREFGYPEN | 83) |
| WP_014561873.1 | EIEQVIEEIN FRRKTKGKNK NPMIKLLPED WVVYYLRKKA LIKPTTKEEL | |
| | IRIIYLFNQR RGFKSSRKDL TETAILDYDE FAKRLAEKEK YSAENYETKF | |
| | VSITKVKEVV ELKTDGRKGK KRFKVILEDS RIEPYEIERK EKPDWEGKEY | |
| | TFLVTQKLEK GKFKQNKPDL PKEEDWALCT TALDNRMGSK HPGEFFFDEL | |
| | LKAFKEKRGY KIRQYPVNRW RYKKELEFIW TKQCQLNPEL NNLNINKEIL | |
| | RKLATVLYPS QSKFFGPKIK EFENSDVLHI ISEDIIYYQR DLKSQKSLIS | |
| | ECRYEKRKGI DGEIYGLKCI PKSSPLYQEF RIWQDIHNIK VIRKESEVNG | |
| | KKKINIDETQ LYINENIKEK LFELFNSKDS LSEKDILELI SLNIINSGIK | |
| | ISKKEEETTH RINLFANRKE LKGNETKSRY RKVFKKLGFD GEYILNHPSK | |
| | LNRLWHSDYS NDYADKEKTE KSILSSLGWK NRNGKWEKSK NYDVFNLPLE | |
| | VAKAIANLPP LKKEYGSYSA LAIRKMLVVM RDGKYWQHPD QIAKDQENTS | |
| | LMLFDKNLIQ LTNNQRKVLN KYLLTLAEVQ KRSTLIKQKL NEIEHNPYKL | |
| | ELVSDQDLEK QVLKSFLEKK NESDYLKGLK TYQAGYLIYG KHSEKDVPIV | |
| | NSPDELGEYI RKKLPNNSLR NPIVEQVIRE TIFIVRDVWK SFGIIDEIHI | |
| | ELGRELKNNS EERKKTSESQ EKNFQEKERA RKLLKELLNS SNFEHYDENG | |
| | NKIFSSFTVN PNPDSPLDIE KFRIWKNQSG LTDEELNKKL KDEKIPTEIE | |
| | VKKYILWLTQ KCRSPYTGKI IPLSKLFDSN VYEIEHIIPR SKMKNDSTNN | |
| | LVICELGVNK AKGDRLAANF ISESNGKCKF GEVEYTLLKY GDYLQYCKDT | |
| | FKYQKAKYKN LLATEPPEDF IERQINDTRY IGRKLAELLT PVVKDSKNII | |
| | FTIGSITSEL KITWGLNGVW KDILRPRFKR LESIINKKLI FQDEDDPNKY | |
| | HFDLSINPQL DKEGLKRLDH RHHALDATII AATTREHVRY LNSLNAADND | |
| | EEKREYFLSL CNHKIRDFKL PWENFTSEVK SKLLSCVVSY KESKPILSDP | |
| | FNKYLKWEYK NGKWQKVFAI QIKNDRWKAV RRSMFKEPIG TVWIKKIKEV | |
| | SLKEAIKIQA IWEEVKNDPV RKKKEKYIYD DYAQKVIAKI VQELGLSSSM | |
| | RKQDDEKLNK FINEAKVSAG VNKNLNTTNK TIYNLEGRFY EKIKVAEYVL | |
| | YKAKRMPLNK KEYIEKLSLQ KMFNDLPNFI LEKSILDNYP EILKELESDN | |
| | KYIIEPHKKN NPVNRLLLEH ILEYHNNPKE AFSTEGLEKL NKKAINKIGK | |
| | PIKYITRLDG DINEEEIFRG AVFETDKGSN VYFVMYENNQ TKDREFLKPN | |
| | PSISVLKAIE HKNKIDFFAP NRLGFSRIIL SPGDLVYVPT NDQYVLIKDN | |
| | SSNETIINWD DNEFISNRIY QVKKFTGNSC YFLKNDIASL ILSYSASNGV | |
| | GEFGSQNISE YSVDDPPIRI KDVCIKIRVD RLGNVRPL | |
| uncultured | MSSKAIDSLE QLDLFKPQEY TLGLDLGIKS IGWAILSGER IANAGVYLFE | (SEQ |
| delta | TAEELNSTGN KLISKAAERG RKRRIRRMLD RKARRGRHIR YLLEREGLPT | ID NO: |
| proteobacterium | DELEEVVVHQ SNRTLWDVRA EAVERKLTKQ ELAAVLFHLV RHRGYFPNTK | 84) |
| HF0070_07E19 | KLPPDDESDS ADEEQGKINR ATSRLREELK ASDCKTIGQF LAQNRDRQRN | |
| ADI19058.1 | REGDYSNLMA RKLVFEEALQ ILAFQRKQGH ELSKDFEKTY LDVLMGQRSG | |
| | RSPKLGNCSL IPSELRAPSS APSTEWFKFL QNLGNLQISN AYREEWSIDA | |
| | PRRAQIIDAC SQRSTSSYWQ IRRDFQIPDE YRFNLVNYER RDPDVDLQEY | |
| | LQQQERKTLA NFRNWKQLEK IIGTGHPIQT LDEAARLITL IKDDEKLSDQ | |
| | LADLLPEASD KAITQLCELD FTTAAKISLE AMYRILPHMN QGMGFFDACQ | |
| | QESLPEIGVP PAGDRVPPFD EMYNPVVNRV LSQSRKLINA VIDEYGMPAK | |
| | IRVELARDLG KGRELRERIK LDQLDKSKQN DQRAEDFRAE FQQAPRGDQS | |
| | LRYRLWKEQN CTCPYSGRMI PVNSVLSEDT QIDHILPISQ SFDNSLSNKV | |
| | LCFTEENAQK SNRTPFEYLD AADFQRLEAI SGNWPEAKRN KLLHKSFGKV | |
| | AEEWKSRALN DTRYLTSALA DHLRHHLPDS KIQTVNGRIT GYLRKQWGLE | |
| | KDRDKHTHHA VDAIVVACTT PAIVQQVTLY HQDIRRYKKL GEKRPTPWPE | |
| | TFRQDVLDVE EEIFITRQPK KVSGGIQTKD TLRKHRSKPD RQRVALTKVK | |
| | LADLERLVEK DASNRNLYEH LKQCLEESGD QPTKAFKAPF YMPSGPEAKQ | |
| | RPILSKVTLL REKPEPPKQL TELSGGRRYD SMAQGRLDIY RYKPGGKRKD | |
| | EYRVVLQRMI DLMRGEENVH VFQKGVPYDQ GPEIEQNYTF LFSLYFDDLV | |
| | EFQRSADSEV IRGYYRTFNI ANGQLKISTY LEGRQDFDFF GANRLAHFAK | |
| | VQVNLLGKVI K | |
| Ruminococcus | MGNYYLGLDV GIGSIGWAVI NIEKKRIEDF NVRIFKSGEI QEKNRNSRAS | (SEQ |
| albus 8 | QQCRRSRGLR RLYRRKSHRK LRLKNYLSII GLTTSEKIDY YYETADNNVI | ID NO: |
| WP_ | QLRNKGLSEK LTPEEIAACL IHICNNRGYK DFYEVNVEDI EDPDERNEYK | 85) |
| 002846926.1 | EEHDSIVLIS NLMNEGGYCT PAEMICNCRE FDEPNSVYRK FHNSAASKNH | |
| | YLITRHMLVK EVDLILENQS KYYGILDDKT IAKIKDIIFA QRDFEIGPGK | |
| | NERFRRFTGY LDSIGKCQFF KDQERGSRFT VIADIYAFVN VLSQYTYTNN | |
| | RGESVFDTSF ANDLINSALK NGSMDKRELK AIAKSYHIDI SDKNSDTSLT | |
| | KCFKYIKWVK PLFEKYGYDW DKLIENYTDT DNNVLNRIGI VLSQAQTPKR | |
| | RREKLKALNI GLDDGLINEL TKLKLSGTAN VSYKYMQGSI EAFCEGDLYG | |
| | KYQAKFNKEI PDIDENAKPQ KLPPFKNEDD CEFFKNPVVF RSINETRKLI | |
| | NAIIDKYGYP AAVNIETADE LNKTFEDRAI DTKRNNDNQK ENDRIVKEII | |
| | ECIKCDEVHA RHLIEKYKLW EAQEGKCLYS GETITKEDML RDKDKLFEVD | |
| | HIVPYSLILD NTINNKALVY AEENQKKGQR TPLMYMNEAQ AADYRVRVNT | |
| | MFKSKKCSKK KYQYLMLPDL NDQELLGGWR SRNLNDTRYI CKYLVNYLRK | |
| | NLRFDRSYES SDEDDLKIRD HYRVFPVKSR FTSMFRRWWL NEKTWGRYDK | |
| | AELKKLTYLD HAADAIIIAN CRPEYWVLAG EKLKLNKMYH QAGKRITPEY | |
| | EQSKKACIDN LYKLFRMDRR TAEKLLSGHG RLTPIIPNLS EEVDKRLWDK | |
| | NIYEQFWKDD KDKKSCEELY RENVASLYKG DPKFASSLSM PVISLKPDHK | |
| | YRGTITGEEA IRVKEIDGKL IKLKRKSISE ITAESINSIY TDDKILIDSL | |
| | KTIFEQADYK DVGDYLKKTN QHFFTTSSGK RVNKVTVIEK VPSRWLRKEI | |
| | DDNNFSLLND SSYYCIELYK DSKGDNNLQG IAMSDIVHDR KTKKLYLKPD | |
| | FNYPDDYYTH VMYIFPGDYL RIKSTSKKSG EQLKFEGYFI SVKNVNENSF | |
| | RFISDNKPCA KDKRVSITKK DIVIKLAVDL MGKVQGENNG KGISCGEPLS | |
| | LLKEKN | |
| Lactobacillus | MTKKEQPYNI GLDIGTSSVG WAVTNDNYDL LNIKKKNLWG VRLFEEAQTA | (SEQ |
| farciminis | KETRLNRSTR RRYRRRKNRI NWLNEIFSEE LAKTDPSFLI RLQNSWVSKK | ID NO: |
| KCTC 3681 | DPDRKRDKYN LFIDGPYTDK EYYREFPTIF HLRKELILNK DKADIRLIYL | 86) |
| WP_ | ALHNILKYRG NFTYEHQKFN ISNLNNNLSK ELIELNQQLI KYDISFPDDC | |
| 010018949.1 | DWNHISDILI GRGNATQKSS NILKDFTLDK ETKKLLKEVI NLILGNVAHL | |
| | NTIFKTSLTK DEEKLNFSGK DIESKLDDLD SILDDDQFTV LDAANRIYST | |
| | ITLNEILNGE SYFSMAKVNQ YENHAIDLCK LRDMWHTTKN EEAVEQSRQA | |
| | YDDYINKPKY GTKELYTSLK KFLKVALPTN LAKEAEEKIS KGTYLVKPRN | |
| | SENGVVPYQL NKIEMEKIID NQSQYYPFLK ENKEKLLSIL SFRIPYYVGP | |
| | LQSAEKNPFA WMERKSNGHA RPWNFDEIVD REKSSNKFIR RMTVTDSYLV | |
| | GEPVLPKNSL IYQRYEVLNE LNNIRITENL KTNPIGSRLT VETKQRIYNE | |
| | LFKKYKKVTV KKLTKWLIAQ GYYKNPILIG LSQKDEFNST LTTYLDMKKI | |
| | FGSSFMEDNK NYDQIEELIE WLTIFEDKQI LNEKLHSSKY SYTPDQIKKI | |
| | SNMRYKGWGR LSKKILMDIT TETNTPQLLQ LSNYSILDLM WATNNNFISI | |
| | MSNDKYDFKN YIENHNLNKN EDQNISDLVN DIHVSPALKR GITQSIKIVQ | |
| | EIVKFMGHAP KHIFIEVTRE TKKSEITTSR EKRIKRLQSK LLNKANDFKP | |
| | QLREYLVPNK KIQEELKKHK NDLSSERIML YFLQNGKSLY SEESLNINKL | |
| | SDYQVDHILP RTYIPDDSLE NKALVLAKEN QRKADDLLLN SNVIDRNLER | |
| | WTYMLNNNMI GLKKFKNLTR RVITDKDKLG FIHRQLVQTS QMVKGVANIL | |
| | DNMYKNQGTT CIQARANLST AFRKALSGQD DTYHFKHPEL VKNRNVNDFH | |
| | HAQDAYLASF LGTYRLRRFP TNEMLLMNGE YNKFYGQVKE LYSKKKKLPD | |
| | SRKNGFIISP LVNGTTQYDR NTGEIIWNVG FRDKILKIFN YHQCNVTRKT | |
| | EIKTGQFYDQ TIYSPKNPKY KKLIAQKKDM DPNIYGGFSG DNKSSITIVK | |
| | IDNNKIKPVA IPIRLINDLK DKKTLQNWLE ENVKHKKSIQ IIKNNVPIGQ | |
| | IIYSKKVGLL SLNSDREVAN RQQLILPPEH SALLRLLQIP DEDLDQILAF | |
| | YDKNILVEIL QELITKMKKF YPFYKGEREF LIANIENFNQ ATTSEKVNSL | |
| | EELITLLHAN STSAHLIFNN IEKKAFGRKT HGLTLNNTDF IYQSVTGLYE | |
| | TRIHIE | |
| Eubacterium | MMEVFMGRLV LGLDIGITSV GFGIIDLDES EIVDYGVRLF KEGTAAENET | (SEQ |
| dolichum DSM | RRTKRGGRRL KRRRVTRRED MLHLLKQAGI ISTSFHPLNN PYDVRVKGLN | ID NO: |
| 3991 | ERLNGEELAT ALLHLCKHRG SSVETIEDDE AKAKEAGETK KVLSMNDQLL | 87) |
| WP_ | KSGKYVCEIQ KERLRTNGHI RGHENNFKTR AYVDEAFQIL SHQDLSNELK | |
| 004800457.1 | SAIITIISRK RMYYDGPGGP LSPTPYGRYT YFGQKEPIDL IEKMRGKCSL | |
| | FPNEPRAPKL AYSAELFNLL NDLNNLSIEG EKLTSEQKAM ILKIVHEKGK | |
| | ITPKQLAKEV GVSLEQIRGF RIDTKGSPLL SELTGYKMIR EVLEKSNDEH | |
| | LEDHVFYDEI AEILTKTKDI EGRKKQISEL SSDLNEESVH QLAGLTKFTA | |
| | YHSLSFKALR LINEEMLKTE LNQMQSITLF GLKQNNELSV KGMKNIQADD | |
| | TAILSPVAKR AQRETFKVVN RLREIYGEFD SIVVEMAREK NSEEQRKAIR | |
| | ERQKFFEMRN KQVADIIGDD RKINAKLREK LVLYQEQDGK TAYSLEPIDL | |
| | KLLIDDPNAY EVDHIIPISI SLDDSITNKV LVTHRENQEK GNLTPISAFV | |
| | KGRFTKGSLA QYKAYCLKLK EKNIKTNKGY RKKVEQYLLN ENDIYKYDIQ | |
| | KEFINRNLVD TSYASRVVLN TLTTYFKQNE IPTKVFTVKG SLTNAFRRKI | |
| | NLKKDRDEDY GHHAIDALII ASMPKMRLLS TIFSRYKIED IYDESTGEVF | |
| | SSGDDSMYYD DRYFAFIASL KAIKVRKFSH KIDTKPNRSV ADETIYSTRV | |
| | IDGKEKVVKK YKDIYDPKFT ALAEDILNNA YQEKYLMALH DPQTFDQIVK | |
| | VVNYYFEEMS KSEKYFTKDK KGRIKISGMN PLSLYRDEHG MLKKYSKKGD | |
| | GPAITQMKYF DGVLGNHIDI SAHYQVRDKK VVLQQISPYR TDFYYSKENG | |
| | YKFVTIRYKD VRWSEKKKKY VIDQQDYAMK KAEKKIDDTY EFQFSMHRDE | |
| | LIGITKAEGE ALIYPDETWH NFNFFFHAGE TPEILKFTAT NNDKSNKIEV | |
| | KPIHCYCKMR LMPTISKKIV RIDKYATDWV GNLYKVKKNT LKFEFD | |
| Nitratifractor | MKKILGVDLG ITSFGYAILQ ETGKDLYRCL DNSVVMRNNP YDEKSGESSQ | (SEQ |
| salsuginis | SIRSTQKSMR RLIEKRKKRI RCVAQTMERY GILDYSETMK INDPKNNPIK | ID NO: |
| DSM 16511 | NRWQLRAVDA WKRPLSPQEL FAIFAHMAKH RGYKSIATED LIYELELELG | 88) |
| ADV46720.1 | LNDPEKESEK KADERRQVYN ALRHLEELRK KYGGETIAQT IHRAVEAGDL | |
| | RSYRNHDDYE KMIRREDIEE EIEKVLLRQA ELGALGLPEE QVSELIDELK | |
| | ACITDQEMPT IDESLFGKCT FYKDELAAPA YSYLYDLYRL YKKLADLNID | |
| | GYEVTQEDRE KVIEWVEKKI AQGKNLKKIT HKDLRKILGL APEQKIFGVE | |
| | DERIVKGKKE PRTFVPFFFL ADIAKFKELF ASIQKHPDAL QIFRELAEIL | |
| | QRSKTPQEAL DRLRALMAGK GIDTDDRELL ELFKNKRSGT RELSHRYILE | |
| | ALPLFLEGYD EKEVQRILGF DDREDYSRYP KSLRHLHLRE GNLFEKEENP | |
| | INNHAVKSLA SWALGLIADL SWRYGPFDEI ILETTRDALP EKIRKEIDKA | |
| | MREREKALDK IIGKYKKEFP SIDKRLARKI QLWERQKGLD LYSGKVINLS | |
| | QLLDGSADIE HIVPQSLGGL STDYNTIVTL KSVNAAKGNR LPGDWLAGNP | |
| | DYRERIGMLS EKGLIDWKKR KNLLAQSLDE IYTENTHSKG IRATSYLEAL | |
| | VAQVLKRYYP FPDPELRKNG IGVRMIPGKV TSKTRSLLGI KSKSRETNFH | |
| | HAEDALILST LTRGWQNRLH RMLRDNYGKS EAELKELWKK YMPHIEGLTL | |
| | ADYIDEAFRR FMSKGEESLF YRDMFDTIRS ISYWVDKKPL SASSHKETVY | |
| | SSRHEVPTLR KNILEAFDSL NVIKDRHKLT TEEFMKRYDK EIRQKLWLHR | |
| | IGNTNDESYR AVEERATQIA QILTRYQLMD AQNDKEIDEK FQQALKELIT | |
| | SPIEVTGKLL RKMRFVYDKL NAMQIDRGLV ETDKNMLGIH ISKGPNEKLI | |
| | FRRMDVNNAH ELQKERSGIL CYLNEMLFIF NKKGLIHYGC LRSYLEKGQG | |
| | SKYIALFNPR FPANPKAQPS KFTSDSKIKQ VGIGSATGII KAHLDLDGHV | |
| | RSYEVFGTLP EGSIEWFKEE SGYGRVEDDP HH | |
| Rhodospirillum | MRPIEPWILG LDIGTDSLGW AVFSCEEKGP PTAKELLGGG VRLFDSGRDA | (SEQ |
| rubrum ATCC | KDHTSRQAER GAFRRARRQT RTWPWRRDRL IALFQAAGLT PPAAETRQIA | ID NO: |
| 11170 | LALRREAVSR PLAPDALWAA LLHLAHHRGF RSNRIDKRER AAAKALAKAK | 89) |
| WP_ | PAKATAKATA PAKEADDEAG FWEGAEAALR QRMAASGAPT VGALLADDLD | |
| 011388212.1 | RGQPVRMRYN QSDRDGVVAP TRALIAEELA EIVARQSSAY PGLDWPAVTR | |
| | LVLDQRPLRS KGAGPCAFLP GEDRALRALP TVQDFIIRQT LANLRLPSTS | |
| | ADEPRPLTDE EHAKALALLS TARFVEWPAL RRALGLKRGV KFTAETERNG | |
| | AKQAARGTAG NLTEAILAPL IPGWSGWDLD RKDRVFSDLW AARQDRSALL | |
| | ALIGDPRGPT RVTEDETAEA VADAIQIVLP TGRASLSAKA ARAIAQAMAP | |
| | GIGYDEAVTL ALGLHHSHRP RQERLARLPY YAAALPDVGL DGDPVGPPPA | |
| | EDDGAAAEAY YGRIGNISVH IALNETRKIV NALLHRHGPI LRLVMVETTR | |
| | ELKAGADERK RMIAEQAERE RENAEIDVEL RKSDRWMANA RERRQRVRLA | |
| | RRQNNLCPYT STPIGHADLL GDAYDIDHVI PLARGGRDSL DNMVLCQSDA | |
| | NKTKGDKTPW EAFHDKPGWI AQRDDFLARL DPQTAKALAW RFADDAGERV | |
| | ARKSAEDEDQ GFLPRQLTDT GYIARVALRY LSLVTNEPNA VVATNGRLTG | |
| | LLRLAWDITP GPAPRDLLPT PRDALRDDTA ARRFLDGLTP PPLAKAVEGA | |
| | VQARLAALGR SRVADAGLAD ALGLTLASLG GGGKNRADHR HHFIDAAMIA | |
| | VTTRGLINQI NQASGAGRIL DLRKWPRTNF EPPYPTFRAE VMKQWDHIHP | |
| | SIRPAHRDGG SLHAATVFGV RNRPDARVLV QRKPVEKLFL DANAKPLPAD | |
| | KIAEIIDGFA SPRMAKRFKA LLARYQAAHP EVPPALAALA VARDPAFGPR | |
| | GMTANTVIAG RSDGDGEDAG LITPFRANPK AAVRTMGNAV YEVWEIQVKG | |
| | RPRWTHRVLT RFDRTQPAPP PPPENARLVM RLRRGDLVYW PLESGDRLFL | |
| | VKKMAVDGRL ALWPARLATG KATALYAQLS CPNINLNGDQ GYCVQSAEGI | |
| | RKEKIRTTSC TALGRLRLSK KAT | |
| Finegoldia | MKSEKKYYIG LDVGTNSVGW AVTDEFYNIL RAKGKDLWGV RLFEKADTAA | (SEQ |
| magna ATCC | NTRIFRSGRR RNDRKGMRLQ ILREIFEDEI KKVDKDFYDR LDESKFWAED | ID NO: |
| 29328 | KKVSGKYSLF NDKNFSDKQY FEKFPTIFHL RKYLMEEHGK VDIRYYFLAI | 90) |
| WP_012290141.1 | NQMMKRRGHF LIDGQISHVT DDKPLKEQLI LLINDLLKIE LEEELMDSIF | |
| | EILADVNEKR TDKKNNLKEL IKGQDFNKQE GNILNSIFES IVTGKAKIKN | |
| | IISDEDILEK IKEDNKEDFV LTGDSYEENL QYFEEVLQEN ITLFNTLKST | |
| | YDFLILQSIL KGKSTLSDAQ VERYDEHKKD LEILKKVIKK YDEDGKLFKQ | |
| | VFKEDNGNGY VSYIGYYLNK NKKITAKKKI SNIEFTKYVK GILEKQCDCE | |
| | DEDVKYLLGK IEQENFLLKQ ISSINSVIPH QIHLFELDKI LENLAKNYPS | |
| | FNNKKEEFTK IEKIRKTFTF RIPYYVGPLN DYHKNNGGNA WIFRNKGEKI | |
| | RPWNFEKIVD LHKSEEEFIK RMLNQCTYLP EETVLPKSSI LYSEYMVLNE | |
| | LNNLRINGKP LDTDVKLKLI EELFKKKTKV TLKSIRDYMV RNNFADKEDF | |
| | DNSEKNLEIA SNMKSYIDFN NILEDKFDVE MVEDLIEKIT IHTGNKKLLK | |
| | KYIEETYPDL SSSQIQKIIN LKYKDWGRLS RKLLDGIKGT KKETEKTDTV | |
| | INFLRNSSDN LMQIIGSQNY SFNEYIDKLR KKYIPQEISY EVVENLYVSP | |
| | SVKKMIWQVI RVTEEITKVM GYDPDKIFIE MAKSEEEKKT TISRKNKLLD | |
| | LYKAIKKDER DSQYEKLLTG LNKLDDSDLR SRKLYLYYTQ MGRDMYTGEK | |
| | IDLDKLFDST HYDKDHIIPQ SMKKDDSIIN NLVLVNKNAN QTTKGNIYPV | |
| | PSSIRNNPKI YNYWKYLMEK EFISKEKYNR LIRNTPLTNE ELGGFINRQL | |
| | VETRQSTKAI KELFEKFYQK SKIIPVKASL ASDLRKDMNT LKSREVNDLH | |
| | HAHDAFLNIV AGDVWNREFT SNPINYVKEN REGDKVKYSL SKDFTRPRKS | |
| | KGKVIWTPEK GRKLIVDTLN KPSVLISNES HVKKGELFNA TIAGKKDYKK | |
| | GKIYLPLKKD DRLQDVSKYG GYKAINGAFF FLVEHTKSKK RIRSIELFPL | |
| | HLLSKFYEDK NTVLDYAINV LQLQDPKIII DKINYRTEII IDNFSYLIST | |
| | KSNDGSITVK PNEQMYWRVD EISNLKKIEN KYKKDAILTE EDRKIMESYI | |
| | DKIYQQFKAG KYKNRRTTDT IIEKYEIIDL DTLDNKQLYQ LLVAFISLSY | |
| | KTSNNAVDFT VIGLGTECGK PRITNLPDNT YLVYKSITGI YEKRIRIK | |
| Eubacterium | MNYTEKEKLF MKYILALDIG IASVGWAILD KESETVIEAG SNIFPEASAA | (SEQ |
| rectale ATCC | DNQLRRDMRG AKRNNRRLKT RINDFIKLWE NNNLSIPQFK STEIVGLKVR | ID NO: |
| 33656 | AITEEITLDE LYLILYSYLK HRGISYLEDA LDDTVSGSSA YANGLKLNAK | 91) |
| WP_ | ELETHYPCEI QQERLNTIGK YRGQSQIINE NGEVLDLSNV FTIGAYRKEI | |
| 012742555.1 | QRVFEIQKKY HPELTDEFCD GYMLIFNRKR KYYEGPGNEK SRTDYGRFTT | |
| | KLDANGNYIT EDNIFEKLIG KCSVYPDELR AAAASYTAQE YNVLNDLNNL | |
| | TINGRKLEEN EKHEIVERIK SSNTINMRKI ISDCMGENID DFAGARIDKS | |
| | GKEIFHKFEV YNKMRKALLE IGIDISNYSR EELDEIGYIM TINTDKEAMM | |
| | EAFQKSWIDL SDDVKQCLIN MRKTNGALFN KWQSFSLKIM NELIPEMYAQ | |
| | PKEQMTLLTE MGVTKGTQEE FAGLKYIPVD VVSEDIFNPV VRRSVRISFK | |
| | ILNAVLKKYK ALDTIVIEMP RDRNSEEQKK RINDSQKLNE KEMEYIEKKL | |
| | AVTYGIKLSP SDFSSQKQLS LKLKLWNEQD GICLYSGKTI DPNDIINNPQ | |
| | LFEIDHIIPR SISFDDARSN KVLVYRSENQ KKGNQTPYYY LTHSHSEWSF | |
| | EQYKATVMNL SKKKEYAISR KKIQNLLYSE DITKMDVLKG FINRNINDTS | |
| | YASRLVLNTI QNFFMANEAD TKVKVIKGSY THQMRCNLKL DKNRDESYSH | |
| | HAVDAMLIGY SELGYEAYHK LQGEFIDFET GEILRKDMWD ENMSDEVYAD | |
| | YLYGKKWANI RNEVVKAEKN VKYWHYVMRK SNRGLCNQTI RGTREYDGKQ | |
| | YKINKLDIRT KEGIKVFAKL AFSKKDSDRE RLLVYLNDRR TFDDLCKIYE | |
| | DYSDAANPFV QYEKETGDII RKYSKKHNGP RIDKLKYKDG EVGACIDISH | |
| | KYGFEKGSKK VILESLVPYR MDVYYKEENH SYYLVGVKQS DIKFEKGRNV | |
| | IDEEAYARIL VNEKMIQPGQ SRADLENLGF KFKLSFYKND IIEYEKDGKI | |
| | YTERLVSRTM PKQRNYIETK PIDKAKFEKQ NLVGLGKTKF IKKYRYDILG | |
| | NKYSCSEEKF TSFC | |
| Corynebacterium | MKYHVGIDVG TFSVGLAAIE VDDAGMPIKT LSLVSHIHDS GLDPDKIKSA | (SEQ |
| diphtheriae | VTRLASSGIA RRTRRLYRRK RRRLQQLDKF IQRQGWPVIE LEDYSDPLYP | ID NO: |
| C7 (beta) | WKVRAELAAS YIADEKERGE KLSVALRHIA RHRGWRNPYA KVSSLYLPDE | 92) |
| AEX66236.1 | PSDAFKAIRE EIKRASGQPV PETATVGQMV TLCELGTLKL RGEGGVLSAR | |
| WP_ | LQQSDHAREI QEICRMQEIG QELYRKIIDV VFAAESPKGS ASSRVGKDPL | |
| 014318431.1 | QPGKNRALKA SDAFQRYRIA ALIGNLRVRV DGEKRILSVE EKNLVFDHLV | |
| | NLAPKKEPEW VTIAEILGID RGQLIGTATM TDDGERAGAR PPTHDTNRSI | |
| | VNSRIAPLVD WWKTASALEQ HAMVKALSNA EVDDFDSPEG AKVQAFFADL | |
| | DDDVHAKLDS LHLPVGRAAY SEDTLVRLTR RMLADGVDLY TARLQEFGIE | |
| | PSWTPPAPRI GEPVGNPAVD RVLKTVSRWL ESATKTWGAP ERVIIEHVRE | |
| | GFVTEKRARE MDGDMRRRAA RNAKLFQEMQ EKLNVQGKPS RADLWRYQSV | |
| | QRQNCQCAYC GSPITFSNSE MDHIVPRAGQ GSTNTRENLV AVCHRCNQSK | |
| | GNTPFAIWAK NTSIEGVSVK EAVERTRHWV TDTGMRSTDF KKFTKAVVER | |
| | FQRATMDEEI DARSMESVAW MANELRSRVA QHFASHGTTV RVYRGSLTAE | |
| | ARRASGISGK LEFLDGVGKS RLDRRHHAID AAVIAFTSDY VAETLAVRSN | |
| | LKQSQAHRQE APQWREFTGK DAEHRAAWRV WCQKMEKLSA LLTEDLRDDR | |
| | VVVMSNVRLR LGNGSAHEET IGKLSKVKLG SQLSVSDIDK ASSEALWCAL | |
| | TREPDFDPKD GLPANPERHI RVNGTHVYAG DNIGLFPVSA GSIALRGGYA | |
| | ELGSSFHHAR VYKITSGKKP AFAMLRVYTI DLLPYRNQDL FSVELKPQTM | |
| | SMRQAEKKLR DALATGNAEY LGWLVVDDEL VVDTSKIATD QVKAVEAELG | |
| | TIRRWRVDGF FGDTRLRLRP LQMSKEGIKK ESAPELSKII DRPGWLPAVN | |
| | KLFSEGNVTV VRRDSLGRVR LESTAHLPVT WKVQ | |
| Roseburia | MNAEHGKEGL LIMEENFQYR IGLDIGITSV GWAVLQNNSQ DEPVRITDLG | (SEQ |
| inulinivorans | VRIFDVAENP KNGDALAAPR RDARTTRRRL RRRRHRLERI KFLLQENGLI | ID NO: |
| DSM 16841 | EMDSFMERYY KGNLPDVYQL RYEGLDRKLK DEELAQVLIH IAKHRGFRST | 93) |
| WP_ | RKAETKEKEG GAVLKATTEN QKIMQEKGYR TVGEMLYLDE AFHTECLWNE | |
| 007889305.1 | KGYVLTPRNR PDDYKHTILR SMLVEEVHAI FAAQRAHGNQ KATEGLEEAY | |
| | VEIMTSQRSF DMGPGLQPDG KPSPYAMEGF GDRVGKCTFE KDEYRAPKAT | |
| | YTAELFVALQ KINHTKLIDE FGTGRFFSEE ERKTIIGLLL SSKELKYGTI | |
| | RKKLNIDPSL KFNSLNYSAK KEGETEEERV LDTEKAKFAS MFWTYEYSKC | |
| | LKDRTEEMPV GEKADLFDRI GEILTAYKND DSRSSRLKEL GLSGEEIDGL | |
| | LDLSPAKYQR VSLKAMRKMQ PYLEDGLIYD KACEAAGYDF RALNDGNKKH | |
| | LLKGEEINAI VNDITNPVVK RSVSQTIKVI NAIIQKYGSP QAVNIELARE | |
| | MSKNFQDRTN LEKEMKKRQQ ENERAKQQII ELGKQNPTGQ DILKYRLWND | |
| | QGGYCLYSGK KIPLEELFDG GYDIDHILPY SITFDDSYRN KVLVTAQENR | |
| | QKGNRTPYEY FGADEKRWED YEASVRLLVR DYKKQQKLLK KNFTEEERKE | |
| | FKERNLNDTK YITRVVYNMI RQNLELEPFN HPEKKKQVWA VNGAVTSYLR | |
| | KRWGLMQKDR STDRHHAMDA VVIACCTDGM IHKISRYMQG RELAYSRNFK | |
| | FPDEETGEIL NRDNFTREQW DEKFGVKVPL PWNSFRDELD IRLLNEDPKN | |
| | FLLTHADVQR ELDYPGWMYG EEESPIEEGR YINYIRPLFV SRMPNHKVTG | |
| | SAHDATIRSA RDYETRGWVI TKVPLTDLKL NKDNEIEGYY DKDSDRLLYQ | |
| | ALVRQLLLHG NDGKKAFAED FHKPKADGTE GPWVRKVKIE KKQTSGVMVR | |
| | GGTGIAANGE MVRIDVFREN GKYYFVPVYT ADVVRKVLPN RAATHTKPYS | |
| | EWRVMDDANF VFSLYSRDLI HVKSKKDIKT NLVNGGLLLQ KEIFAYYTGA | |
| | DIATASIAGF ANDSNFKFRG LGIQSLEIFE KCQVDILGNI SVVRHENRQE | |
| | FH | |
| Alicycliphilus | MRSLRYRLAL DLGSTSLGWA LFRLDACNRP TAVIKAGVRI FSDGRNPKDG | (SEQ |
| denitrificans | SSLAVTRRAA RAMRRRRDRL LKRKTRMQAK LVEHGFFPAD AGKRKALEQL | ID NO: |
| K601 | NPYALRAKGL QEALLPGEFA RALFHINQRR GFKSNRKTDK KDNDSGVLKK | 94) |
| WP_ | AIGQLRQQMA EQGSRTVGEY LWTRLQQGQG VRARYREKPY TTEEGKKRID | |
| 013517127.1 | KSYDLYIDRA MIEQEFDALW AAQAAFNPTL FHEAARADLK DTLLHQRPLR | |
| | PVKPGRCTLL PEEERAPLAL PSTQRFRIHQ EVNHLRLLDE NLREVALTLA | |
| | QRDAVVTALE TKAKLSFEQI RKLLKLSGSV QFNLEDAKRT ELKGNATSAA | |
| | LARKELFGAA WSGFDEALQD EIVWQLVTEE GEGALIAWLQ THTGVDEARA | |
| | QAIVDVSLPE GYGNLSRKAL ARIVPALRAA VITYDKAVQA AGFDHHSQLG | |
| | FEYDASEVED LVHPETGEIR SVFKQLPYYG KALQRHVAFG SGKPEDPDEK | |
| | RYGKIANPTV HIGLNQVRMV VNALIRRYGR PTEVVIELAR DLKQSREQKV | |
| | EAQRRQADNQ RRNARIRRSI AEVLGIGEER VRGSDIQKWI CWEELSFDAA | |
| | DRRCPYSGVQ ISAAMLLSDE VEVEHILPFS KTLDDSLNNR TVAMRQANRI | |
| | KRNRTPWDAR AEFEAQGWSY EDILQRAERM PLRKRYRFAP DGYERWLGDD | |
| | KDFLARALND TRYLSRVAAE YLRLVCPGTR VIPGQLTALL RGKFGLNDVL | |
| | GLDGEKNRND HRHHAVDACV IGVTDQGLMQ RFATASAQAR GDGLTRLVDG | |
| | MPMPWPTYRD HVERAVRHIW VSHRPDHGFE GAMMEETSYG IRKDGSIKQR | |
| | RKADGSAGRE ISNLIRIHEA TQPLRHGVSA DGQPLAYKGY VGGSNYCIEI | |
| | TVNDKGKWEG EVISTFRAYG VVRAGGMGRL RNPHEGQNGR KLIMRLVIGD | |
| | SVRLEVDGAE RTMRIVKISG SNGQIFMAPI HEANVDARNT DKQDAFTYTS | |
| | KYAGSLQKAK TRRVTISPIGEVRDPGFKG | |
| Sphaerochaeta | MSKKVSRRYE EQAQEICQRL GSRPYSIGLD LGVGSIGVAV AAYDPIKKQP | (SEQ |
| globosa str. | SDLVFVSSRI FIPSTGAAER RQKRGQRNSL RHRANRLKFL WKLLAERNLM | ID NO: |
| Buddy | LSYSEQDVPD PARLRFEDAV VRANPYELRL KGLNEQLTLS ELGYALYHIA | 95) |
| WP_ | NHRGSSSVRT FLDEEKSSDD KKLEEQQAMT EQLAKEKGIS TFIEVLTAFN | |
| 013607849.1 | TNGLIGYRNS ESVKSKGVPV PTRDIISNEI DVLLQTQKQF YQEILSDEYC | |
| | DRIVSAILFE NEKIVPEAGC CPYFPDEKKL PRCHFLNEER RLWEAINNAR | |
| | IKMPMQEGAA KRYQSASFSD EQRHILFHIA RSGTDITPKL VQKEFPALKT | |
| | SIIVLQGKEK AIQKIAGFRF RRLEEKSFWK RLSEEQKDDF FSAWTNTPDD | |
| | KRLSKYLMKH LLLTENEVVD ALKTVSLIGD YGPIGKTATQ LLMKHLEDGL | |
| | TYTEALERGM ETGEFQELSV WEQQSLLPYY GQILTGSTQA LMGKYWHSAF | |
| | KEKRDSEGFF KPNTNSDEEK YGRIANPVVH QTLNELRKLM NELITILGAK | |
| | PQEITVELAR ELKVGAEKRE DIIKQQTKQE KEAVLAYSKY CEPNNLDKRY | |
| | IERFRLLEDQ AFVCPYCLEH ISVADIAAGR ADVDHIFPRD DTADNSYGNK | |
| | VVAHRQCNDI KGKRTPYAAF SNTSAWGPIM HYLDETPGMW RKRRKFETNE | |
| | EEYAKYLQSK GFVSRFESDN SYIAKAAKEY LRCLFNPNNV TAVGSLKGME | |
| | TSILRKAWNL QGIDDLLGSR HWSKDADTSP TMRKNRDDNR HHGLDAIVAL | |
| | YCSRSLVQMI NTMSEQGKRA VEIEAMIPIP GYASEPNLSF EAQRELFRKK | |
| | ILEFMDLHAF VSMKTDNDAN GALLKDTVYS ILGADTQGED LVFVVKKKIK | |
| | DIGVKIGDYE EVASAIRGRI TDKQPKWYPM EMKDKIEQLQ SKNEAALQKY | |
| | KESLVQAAAV LEESNRKLIE SGKKPIQLSE KTISKKALEL VGGYYYLISN | |
| | NKRTKTFWVK EPSNEVKGFA FDTGSNLCLD FYHDAQGKLC GEIIRKIQAM | |
| | NPSYKPAYMK QGYSLYVRLY QGDVCELRAS DLTEAESNLA KTTHVRLPNA | |
| | KPGRTFVIII TFTEMGSGYQ IYFSNLAKSK KGQDTSFTLT TIKNYDVRKV | |
| | QLSSAGLVRY VSPLLVDKIE KDEVALCGE | |
| Fusobacterium | MKKQKFSDYY LGFDIGTNSV GWCVTDLDYN VLRFNKKDMW GSRLFDEAKT | (SEQ |
| nucleatum | AAERRVQRNS RRRLKRRKWR LNLLEEIFSD EIMKIDSNFF RRLKESSLWL | ID NO: |
| subsp. | EDKNSKEKFT LFNDDNYKDY DFYKQYPTIF HLRDELIKNP EKKDIRLIYL | |
| vincentii ATCC | ALHSIFKSRG HFLFEGQNLK EIKNFETLYN NLISFLEDNG INKSIDKDNI | 96) |
| 49256 | EKLEKIICDS GKGLKDKEKE FKGIFNSDKQ LVAIFKLSVG SSVSLNDLFD | |
| WP_ | TDEYKKEEVE KEKISFREQI YEDDKPIYYS ILGEKIELLD IAKSFYDFMV | |
| 005888649.1 | LNNILSDSNY ISEAKVKLYE EHKKDLKNLK YIIRKYNKEN YDKLFKDKNE | |
| | NNYPAYIGLN KEKDKKEVVE KSRLKIDDLI KVIKGYLPKP ERIEEKDKTI | |
| | FNEILNKIEL KTILPKQRIS DNGTLPYQIH EVELEKILEN QSKYYDFLNY | |
| | EENGVSTKDK LLKTFKFRIP YYVGPLNSYH KDKGGNSWIV RKEEGKILPW | |
| | NFEQKVDIEK SAEEFIKRMT NKCTYLNGED VIPKDSFLYS EYIILNELNK | |
| | VQVNDEFLNE ENKRKIIDEL FKENKKVSEK KFKEYLLVNQ IANRTVELKG | |
| | IKDSFNSNYV SYIKFKDIFG EKLNLDIYKE ISEKSILWKC LYGDDKKIFE | |
| | KKIKNEYGDI LNKDEIKKIN SFKFNTWGRL SEKLLTGIEF INLETGECYS | |
| | SVMEALRRTN YNLMELLSSK FTLQESIDNE NKEMNEVSYR DLIEESYVSP | |
| | SLKRAILQTL KIYEEIKKIT GRVPKKVFIE MARGGDESMK NKKIPARQEQ | |
| | LKKLYDSCGN DIANFSIDIK EMKNSLSSYD NNSLRQKKLY LYYLQFGKCM | |
| | YTGREIDLDR LLQNNDTYDI DHIYPRSKVI KDDSFDNLVL VLKNENAEKS | |
| | NEYPVKKEIQ EKMKSFWRFL KEKNFISDEK YKRLTGKDDF ELRGFMARQL | |
| | VNVRQTTKEV GKILQQIEPE IKIVYSKAEI ASSFREMFDF IKVRELNDTH | |
| | HAKDAYLNIV AGNVYNTKFT EKPYRYLQEI KENYDVKKIY NYDIKNAWDK | |
| | ENSLEIVKKN MEKNTVNITR FIKEEKGELF NLNPIKKGET SNEIISIKPK | |
| | LYDGKDNKLN EKYGYYTSLK AAYFIYVEHE KKNKKVKTFE RITRIDSTLI | |
| | KNEKNLIKYL VSQKKLLNPK IIKKIYKEQT LIIDSYPYTF TGVDSNKKVE | |
| | LKNKKQLYLE KKYEQILKNA LKFVEDNQGE TEENYKFIYL KKRNNNEKNE | |
| | TIDAVKERYN IEFNEMYDKF LEKLSSKDYK NYINNKLYTN FLNSKEKFKK | |
| | LKLWEKSLIL REFLKIFNKN TYGKYEIKDS QTKEKLFSFP EDTGRIRLGQ | |
| | SSLGNNKELL EESVTGLFVK KIKL | |
| Pasteurella | MQTTNLSYIL GLDLGIASVG WAVVEINENE DPIGLIDVGV RIFERAEVPK | (SEQ |
| multocida | TGESLALSRR LARSTRRLIR RRAHRLLLAK RFLKREGILS TIDLEKGLPN | ID NO: |
| subsp. | QAWELRVAGL ERRLSAIEWG AVLLHLIKHR GYLSKRKNES QTNNKELGAL | 97) |
| multocida str. | LSGVAQNHQL LQSDDYRTPA ELALKKFAKE EGHIRNQRGA YTHTFNRLDL | |
| Pm70 | LAELNLLFAQ QHQFGNPHCK EHIQQYMTEL LMWQKPALSG EAILKMLGKC | |
| WP_010907033.1 | THEKNEFKAA KHTYSAERFV WLTKLNNLRI LEDGAERALN EEERQLLINH | |
| | PYEKSKLTYA QVRKLLGLSE QAIFKHLRYS KENAESATFM ELKAWHAIRK | |
| | ALENQGLKDT WQDLAKKPDL LDEIGTAFSL YKTDEDIQQY LTNKVPNSVI | |
| | NALLVSLNFD KFIELSLKSL RKILPLMEQG KRYDQACREI YGHHYGEANQ | |
| | KTSQLLPAIP AQEIRNPVVL RTLSQARKVI NAIIRQYGSP ARVHIETGRE | |
| | LGKSFKERRE IQKQQEDNRT KRESAVQKFK ELFSDFSSEP KSKDILKFRL | |
| | YEQQHGKCLY SGKEINIHRL NEKGYVEIDH ALPFSRTWDD SFNNKVLVLA | |
| | SENQNKGNQT PYEWLQGKIN SERWKNFVAL VLGSQCSAAK KQRLLTQVID | |
| | DNKFIDRNLN DTRYIARFLS NYIQENLLLV GKNKKNVFTP NGQITALLRS | |
| | RWGLIKAREN NNRHHALDAI VVACATPSMQ QKITRFIRFK EVHPYKIENR | |
| | YEMVDQESGE IISPHFPEPW AYFRQEVNIR VFDNHPDTVL KEMLPDRPQA | |
| | NHQFVQPLFV SRAPTRKMSG QGHMETIKSA KRLAEGISVL RIPLTQLKPN | |
| | LLENMVNKER EPALYAGLKA RLAEFNQDPA KAFATPFYKQ GGQQVKAIRV | |
| | EQVQKSGVLV RENNGVADNA SIVRTDVFIK NNKFFLVPIY TWQVAKGILP | |
| | NKAIVAHKNE DEWEEMDEGA KFKFSLFPND LVELKTKKEY FFGYYIGLDR | |
| | ATGNISLKEH DGEISKGKDG VYRVGVKLAL SFEKYQVDEL GKNRQICRPQ | |
| | QRQPVR | |
| Alcanivorax | MRYRVGLDLG TASVGAAVFS MDEQGNPMEL IWHYERLFSE PLVPDMGQLK | (SEQ |
| pacificus | PKKAARRLAR QQRRQIDRRA SRLRRIAIVS RRLGIAPGRN DSGVHGNDVP | ID NO: |
| W11-5 | TLRAMAVNER IELGQLRAVL LRMGKKRGYG GTFKAVRKVG EAGEVASGAS | 98) |
| WP_00873826 | RLEEEMVALA SVQNKDSVTV GEYLAARVEH GLPSKLKVAA NNEYYAPEYA | |
| 9.1 | LFRQYLGLPA IKGRPDCLPN MYALRHQIEH EFERIWATQS QFHDVMKDHG | |
| | VKEEIRNAIF FQRPLKSPAD KVGRCSLQTN LPRAPRAQIA AQNFRIEKQM | |
| | ADLRWGMGRR AEMLNDHQKA VIRELLNQQK ELSFRKIYKE LERAGCPGPE | |
| | GKGLNMDRAA LGGRDDLSGN TTLAAWRKLG LEDRWQELDE VTQIQVINFL | |
| | ADLGSPEQLD TDDWSCRFMG KNGRPRNFSD EFVAFMNELR MTDGFDRLSK | |
| | MGFEGGRSSY SIKALKALTE WMIAPHWRET PETHRVDEEA AIRECYPESL | |
| | ATPAQGGRQS KLEPPPLTGN EVVDVALRQV RHTINMMIDD LGSVPAQIVV | |
| | EMAREMKGGV TRRNDIEKQN KRFASERKKA AQSIEENGKT PTPARILRYQ | |
| | LWIEQGHQCP YCESNISLEQ ALSGAYTNFE HILPRTLTQI GRKRSELVLA | |
| | HRECNDEKGN RTPYQAFGHD DRRWRIVEQR ANALPKKSSR KTRLLLLKDF | |
| | EGEALTDESI DEFADRQLHE SSWLAKVTTQ WLSSLGSDVY VSRGSLTAEL | |
| | RRRWGLDTVI PQVRFESGMP VVDEEGAEIT PEEFEKFRLQ WEGHRVTREM | |
| | RTDRRPDKRI DHRHHLVDAI VTALTSRSLY QQYAKAWKVA DEKQRHGRVD | |
| | VKVELPMPIL TIRDIALEAV RSVRISHKPD RYPDGRFFEA TAYGIAQRLD | |
| | ERSGEKVDWL VSRKSLTDLA PEKKSIDVDK VRANISRIVG EAIRLHISNI | |
| | FEKRVSKGMT PQQALREPIE FQGNILRKVR CFYSKADDCV RIEHSSRRGH | |
| | HYKMLLNDGF AYMEVPCKEG ILYGVPNLVR PSEAVGIKRA PESGDFIRFY | |
| | KGDTVKNIKT GRVYTIKQIL GDGGGKLILT PVTETKPADL LSAKWGRLKV | |
| | GGRNIHLLRL CAE | |
| Mycoplasma | MYFYKNKENK LNKKVVLGLD LGIASVGWCL TDISQKEDNK FPIILHGVRL | (SEQ |
| mobile 163K | FETVDDSDDK LLNETRRKKR GQRRRNRRLF TRKRDFIKYL IDNNIIELEF | ID NO: |
| AAT27519.1 | DKNPKILVRN FIEKYINPFS KNLELKYKSV TNLPIGFHNL RKAAINEKYK | 99) |
| | LDKSELIVLL YFYLSLRGAF FDNPEDTKSK EMNKNEIEIF DKNESIKNAE | |
| | FPIDKIIEFY KISGKIRSTI NLKFGHQDYL KEIKQVFEKQ NIDFMNYEKF | |
| | AMEEKSFFSR IRNYSEGPGN EKSFSKYGLY ANENGNPELI INEKGQKIYT | |
| | KIFKTLWESK IGKCSYDKKL YRAPKNSFSA KVFDITNKLT DWKHKNEYIS | |
| | ERLKRKILLS RFLNKDSKSA VEKILKEENI KFENLSEIAY NKDDNKINLP | |
| | IINAYHSLTT IFKKHLINFE NYLISNENDL SKLMSFYKQQ SEKLFVPNEK | |
| | GSYEINQNNN VLHIFDAISN ILNKFSTIQD RIRILEGYFE FSNLKKDVKS | |
| | SEIYSEIAKL REFSGTSSLS FGAYYKFIPN LISEGSKNYS TISYEEKALQ | |
| | NQKNNFSHSN LFEKTWVEDL IASPTVKRSL RQTMNLLKEI FKYSEKNNLE | |
| | IEKIVVEVTR SSNNKHERKK IEGINKYRKE KYEELKKVYD LPNENTTLLK | |
| | KLWLLRQQQG YDAYSLRKIE ANDVINKPWN YDIDHIVPRS ISFDDSFSNL | |
| | VIVNKLDNAK KSNDLSAKQF IEKIYGIEKL KEAKENWGNW YLRNANGKAF | |
| | NDKGKFIKLY TIDNLDEFDN SDFINRNLSD TSYITNALVN HLTFSNSKYK | |
| | YSWVSVNGKQ TSNLRNQIAF VGIKNNKETE REWKRPEGFK SINSNDFLIR | |
| | EEGKNDVKDD VLIKDRSFNG HHAEDAYFIT IISQYFRSFK RIERLNVNYR | |
| | KETRELDDLE KNNIKFKEKA SFDNFLLINA LDELNEKLNQ MRFSRMVITK | |
| | KNTQLFNETL YSGKYDKGKN TIKKVEKLNL LDNRTDKIKK IEEFFDEDKL | |
| | KENELTKLHI FNHDKNLYET LKIIWNEVKI EIKNKNLNEK NYFKYFVNKK | |
| | LQEGKISFNE WVPILDNDFK IIRKIRYIKF SSEEKETDEI IFSQSNFLKI | |
| | DQRQNFSFHN TLYWVQIWVY KNQKDQYCFI SIDARNSKFE KDEIKINYEK | |
| | LKTQKEKLQI INEEPILKIN KGDLFENEEK ELFYIVGRDE KPQKLEIKYI | |
| | LGKKIKDQKQ IQKPVKKYFP NWKKVNLTYM GEIFKK | |
| gamma | MTKNYISPIA IDLGAKFTGV ALYQYLEGAD CTQEVAKGLL VDDRGNVTWS | (SEQ |
| proteobacterium | QEGRRGKRHQ VRGYKRRKMA KRLLWLILDS EYGIKREEVT EPLLKFINGL | ID NO: |
| HTCC5015 | LNRRGYTYIS EEVDEESMNV SPLPFSEMMP DYFNSSAPLL EQLAKLLSDK | 100) |
| WP_ | NKLVRFRAEG KIPSNKNEFK KLLDTALDGK YKDEKKELSE AWGNILIASE | |
| 008284239.1 | NVLKSTVDGH KSRSEYLANI KEDIKSNEEL EKQISSKEID GFYNLVGHLS | |
| | NFQLRLLRKY FNDPNMSGVS YWDEKRLEKY FYQWVQGWHT KGGTDEAEKK | |
| | NIILKTKGAP LLKTLKSLSA DLTIPPYEDQ NNRRPPKCQS VLLSDEKLTM | |
| | HYPKWKEWVG QLVKQNDNAY LNENVTLANA LHRIVERSRS IDPYQLRLLI | |
| | SITDAEKRND LAGYKRLKLS LGSEVDEFLL LVKNIVDETK EAREGLWFET | |
| | ENKLFFKCGK TPPRKEKLKS TLLSAVLGKN LSDDEQSSFI EEFWKSGTPK | |
| | IERRNVRGWC RLASQVQKTY GVYLKEYGLQ QLHKLEAGKK LDDKPLALLY | |
| | KNSGLIASKI GEALNIEPDE VSRFASPHSL AQIFNIIEGD VAGFNKTCRA | |
| | CTYENIWRMQ EEKVESLLTN QLLSEIHGER KVPLKSAMCT RLSADSTRPF | |
| | DGQMASIIEH IARKIAQHKI AQINDVPKEF SIDIPIIIES NQFSFTAELE | |
| | EIKRGRGSAK AKKAKELGEK SKAGWVSKTE RIKTSSEGIC PYTGAPLGGS | |
| | GEIDHIIPRS LTGRTKKTVF NSEANLIYCS SKGNHDKGNR VYVIEQLNDK | |
| | YLKKQFSTSD VNLIKKKIKT TIQRFTEGGE KLRSFSELSR EDQKAFRHAL | |
| | FVPELKSEVT SLLAVKNITR VNGTQAWLAK KIASLLAEHL DKQGRDYTLS | |
| | AHQIDPWSVS KQRKMLASAE PIWAKKDPQP AASHVVDAVC TFLEALEQPH | |
| | TASRLKTISS TSFEKTGWRS ALIPDLIKVD ALDRRPKYRR YNIGSTSLFK | |
| | DGIYAERFLP ILIDENGLMA GYDIDNSLKA KGADVVFESL SPFLLFKGEE | |
| | VGAQSLSDWQ ERIDGRYLYM SIDKVKAFDY LQEKVGEKDI AAELLNSIHF | |
| | TQRKTELRAK FSDDSGKKMK TLDAIRKSLK LTVTVNEIGK RKEKCGFSGT | |
| | IGIPAKSAWE NLLDEPLLET YWGTKMPPQE IWEKVYRKHF PRNIPNQAHR | |
| | KVRKDFSLPV VDSVSGGFRV KRKTPNGYNY QLLAIDGYSA VGFKKEGDNV | |
| | DFKSPALVPQ IAESKSVTPI SSELVHLDKN EIVYFDEWRK IDISDSDLKQ | |
| | FVSSLELAPG SQNRFYIRFT VDEDQFERHF KSALRVNGIQ DLDTVNKTFD | |
| | WNREIPSLLI PPRSNLFLLE TGQKITFEYI ANGANAEVKK AYSLRRA | |
| Planococcus | MKNYTIGLDI GVASVGWVCI DENYKILNYN NRHAFGVHEF ESAESAAGRR | (SEQ |
| antarcticus | LKRGMRRRYN RRKKRLQLLQ SLFDSYITDS GFFSKTDSQH FWKNNNEFEN | ID NO: |
| DSM 14505 | RSLTEVLSSL RISSRKYPTI YHLRSDLIES NKKMDLRLVY LALHNLVKYR | 101) |
| ANU10858.1 | GHFLQEGNWS EAASAEGMDD QLLELVTRYA ELENLSPLDL SESQWKAAET | |
| | LLLNRNLTKT DQSKELTAMF GKEYEPFCKL VAGLGVSLHQ LFPSSEQALA | |
| | YKETKTKVQL SNENVEEVME LLLEEESALL EAVQPFYQQV VLYELLKGET | |
| | YVAKAKVSAF KQYQKDMASL KNLLDKTFGE KVYRSYFISD KNSQREYQKS | |
| | HKVEVLCKLD QFNKEAKFAE TFYKDLKKLL EDKSKTSIGT TEKDEMLRII | |
| | KAIDSNQFLQ KQKGIQNAAI PHQNSLYEAE KILRNQQAHY PFITTEWIEK | |
| | VKQILAFRIP YYIGPLVKDT TQSPFSWVER KGDAPITPWN FDEQIDKAAS | |
| | AEAFISRMRK TCTYLKGQEV LPKSSLTYER FEVLNELNGI QLRTTGAESD | |
| | FRHRLSYEMK CWIIDNVFKQ YKTVSTKRLL QELKKSPYAD ELYDEHTGEI | |
| | KEVFGTQKEN AFATSLSGYI SMKSILGAVV DDNPAMTEEL IYWIAVFEDR | |
| | EILHLKIQEK YPSITDVQRQ KLALVKLPGW GRFSRLLIDG LPLDEQGQSV | |
| | LDHMEQYSSV FMEVLKNKGF GLEKKIQKMN QHQVDGTKKI RYEDIEELAG | |
| | SPALKRGIWR SVKIVEELVS IFGEPANIVL EVAREDGEKK RTKSRKDQWE | |
| | ELTKTTLKND PDLKSFIGEI KSQGDQRFNE QRFWLYVTQQ GKCLYTGKAL | |
| | DIQNLSMYEV DHILPQNFVK DDSLDNLALV MPEANQRKNQ VGQNKMPLEI | |
| | IEANQQYAMR TLWERLHELK LISSGKLGRL KKPSFDEVDK DKFIARQLVE | |
| | TRQIIKHVRD LLDERFSKSD IHLVKAGIVS KFRRFSEIPK IRDYNNKHHA | |
| | MDALFAAALI QSILGKYGKN FLAFDLSKKD RQKQWRSVKG SNKEFFLFKN | |
| | FGNLRLQSPV TGEEVSGVEY MKHVYFELPW QTTKMTQTGD GMFYKESIFS | |
| | PKVKQAKYVS PKTEKFVHDE VKNHSICLVE FTFMKKEKEV QETKFIDLKV | |
| | IEHHQFLKEP ESQLAKFLAE KETNSPIIHA RIIRTIPKYQ KIWIEHFPYY | |
| | FISTRELHNA RQFEISYELM EKVKQLSERS SVEELKIVFG LLIDQMNDNY | |
| | PIYTKSSIQD RVQKFVDTQL YDFKSFEIGF EELKKAVAAN AQRSDTFGSR | |
| | ISKKPKPEEV AIGYESITGL KYRKPRSVVG TKR | |
| Prevotella sp. | MTQKVLGLDL GTNSIGSAVR NLDLSDDLQW QLEFFSSDIF RSSVNKESNG | (SEQ |
| C561 | REYSLAAQRS AHRRSRGLNE VRRRRLWATL NLLIKHGFCP MSSESLMRWC | ID NO: |
| WP_00901330 | TYDKRKGLFR EYPIDDKDFN AWILLDFNGD GRPDYSSPYQ LRRELVTRQF | 102) |
| 3.1 | DFEQPIERYK LGRALYHIAQ HRGFKSSKGE TLSQQETNSK PSSTDEIPDV | |
| | AGAMKASEEK LSKGLSTYMK EHNLLTVGAA FAQLEDEGVR VRNNNDYRAI | |
| | RSQFQHEIET IFKFQQGLSV ESELYERLIS EKKNVGTIFY KRPLRSQRGN | |
| | VGKCTLERSK PRCAIGHPLF EKFRAWTLIN NIKVRMSVDT LDEQLPMKLR | |
| | LDLYNECFLA FVRTEFKFED IRKYLEKRLG IHFSYNDKTI NYKDSTSVAG | |
| | CPITARFRKM LGEEWESFRV EGQKERQAHS KNNISFHRVS YSIEDIWHFC | |
| | YDAEEPEAVL AFAQETLRLE RKKAEELVRI WSAMPQGYAM LSQKAIRNIN | |
| | KILMLGLKYS DAVILAKVPE LVDVSDEELL SIAKDYYLVE AQVNYDKRIN | |
| | SIVNGLIAKY KSVSEEYRFA DHNYEYLLDE SDEKDIIRQI ENSLGARRWS | |
| | LMDANEQTDI LQKVRDRYQD FFRSHERKFV ESPKLGESFE NYLTKKFPMV | |
| | EREQWKKLYH PSQITIYRPV SVGKDRSVLR LGNPDIGAIK NPTVLRVLNT | |
| | LRRRVNQLLD DGVISPDETR VVVETARELN DANRKWALDT YNRIRHDENE | |
| | KIKKILEEFY PKRDGISTDD IDKARYVIDQ REVDYFTGSK TYNKDIKKYK | |
| | FWLEQGGQCM YTGRTINLSN LFDPNAFDIE HTIPESLSFD SSDMNLTLCD | |
| | AHYNRFIKKN HIPTDMPNYD KAITIDGKEY PAITSQLQRW VERVERLNRN | |
| | VEYWKGQARR AQNKDRKDQC MREMHLWKME LEYWKKKLER FTVTEVTDGF | |
| | KNSQLVDTRV ITRHAVLYLK SIFPHVDVQR GDVTAKFRKI LGIQSVDEKK | |
| | DRSLHSHHAI DATTLTIIPV SAKRDRMLEL FAKIEEINKM LSFSGSEDRT | |
| | GLIQELEGLK NKLQMEVKVC RIGHNVSEIG TFINDNIIVN HHIKNQALTP | |
| | VRRRLRKKGY IVGGVDNPRW QTGDALRGEI HKASYYGAIT QFAKDDEGKV | |
| | LMKEGRPQVN PTIKFVIRRE LKYKKSAADS GFASWDDLGK AIVDKELFAL | |
| | MKGQFPAETS FKDACEQGIY MIKKGKNGMP DIKLHHIRHV RCEAPQSGLK | |
| | IKEQTYKSEK EYKRYFYAAV GDLYAMCCYT NGKIREFRIY SLYDVSCHRK | |
| | SDIEDIPEFI TDKKGNRLML DYKLRTGDMI LLYKDNPAEL YDLDNVNLSR | |
| | RLYKINRFES QSNLVLMTHH LSTSKERGRS LGKTVDYQNL PESIRSSVKS | |
| | LNFLIMGENR DFVIKNGKII FNHR | |
| Alicyclobacillus | MAYRLGLDIG ITSVGWAVVA LEKDESGLKP VRIQDLGVRI FDKAEDSKTG | (SEQ |
| hesperidum | ASLALPRREA RSARRRTRRR RHRLWRVKRL LEQHGILSME QIEALYAQRT | ID NO: |
| URH17-3-68 | SSPDVYALRV AGLDRCLIAE EIARVLIHIA HRRGFQSNRK SEIKDSDAGK | 103) |
| WP_006446566.1 | LLKAVQENEN LMQSKGYRTV AEMLVSEATK TDAEGKLVHG KKHGYVSNVR | |
| | NKAGEYRHTV SRQAIVDEVR KIFAAQRALG NDVMSEELED SYLKILCSQR | |
| | NFDDGPGGDS PYGHGSVSPD GVRQSIYERM VGSCTFETGE KRAPRSSYSF | |
| | ERFQLLTKVV NLRIYRQQED GGRYPCELTQ TERARVIDCA YEQTKITYGK | |
| | LRKLLDMKDT ESFAGLTYGL NRSRNKTEDT VFVEMKFYHE VRKALQRAGV | |
| | FIQDLSIETL DQIGWILSVW KSDDNRRKKL STLGLSDNVI EELLPLNGSK | |
| | FGHLSLKAIR KILPFLEDGY SYDVACELAG YQFQGKTEYV KQRLLPPLGE | |
| | GEVTNPVVRR ALSQAIKVVN AVIRKHGSPE SIHIELAREL SKNLDERRKI | |
| | EKAQKENQKN NEQIKDEIRE ILGSAHVTGR DIVKYKLFKQ QQEFCMYSGE | |
| | KLDVTRLFEP GYAEVDHIIP YGISFDDSYD NKVLVKTEQN RQKGNRTPLE | |
| | YLRDKPEQKA KFIALVESIP LSQKKKNHLL MDKRAIDLEQ EGFRERNLSD | |
| | TRYITRALMN HIQAWLLFDE TASTRSKRVV CVNGAVTAYM RARWGLTKDR | |
| | DAGDKHHAAD AVWVACIGDS LIQRVTKYDK FKRNALADRN RYVQQVSKSE | |
| | GITQYVDKET GEVFTWESFD ERKFLPNEPL EPWPFFRDEL LARLSDDPSK | |
| | NIRAIGLLTY SETEQIDPIF VSRMPTRKVT GAAHKETIRS PRIVKVDDNK | |
| | GTEIQVVVSK VALTELKLTK DGEIKDYFRP EDDPRLYNTL RERLVQFGGD | |
| | AKAAFKEPVY KISKDGSVRT PVRKVKIQEK LTLGVPVHGG RGIAENGGMV | |
| | RIDVFAKGGK YYFVPIYVAD VLKRELPNRL ATAHKPYSEW RVVDDSYQFK | |
| | FSLYPNDAVM IKPSREVDIT YKDRKEPVGC RIMYFVSANI ASASISLRTH | |
| | DNSGELEGLG IQGLEVFEKY WVGPLGDTHP VYKERRMPFR VERKMN | |
| Lactobacillus | MTKLNQPYGI GLDIGSNSIG FAVVDANSHL LRLKGETAIG ARLFREGQSA | (SEQ |
| rhamnosus GG | ADRRGSRTTR RRLSRTRWRL SFLRDFFAPH ITKIDPDFFL RQKYSEISPK | ID NO: |
| WP_014569977.1 | DKDRFKYEKR LFNDRTDAEF YEDYPSMYHL RLHLMTHTHK ADPREIFLAI | 104) |
| | HHILKSRGHF LTPGAAKDFN TDKVDLEDIF PALTEAYAQV YPDLELTFDL | |
| | AKADDFKAKL LDEQATPSDT QKALVNLLLS SDGEKEIVKK RKQVLTEFAK | |
| | AITGLKTKFN LALGTEVDEA DASNWQFSMG QLDDKWSNIE TSMTDQGTEI | |
| | FEQIQELYRA RLLNGIVPAG MSLSQAKVAD YGQHKEDLEL FKTYLKKLND | |
| | HELAKTIRGL YDRYINGDDA KPFLREDFVK ALTKEVTAHP NEVSEQLLNR | |
| | MGQANFMLKQ RTKANGAIPI QLQQRELDQI IANQSKYYDW LAAPNPVEAH | |
| | RWKMPYQLDE LLNFHIPYYV GPLITPKQQA ESGENVFAWM VRKDPSGNIT | |
| | PYNFDEKVDR EASANTFIQR MKTTDTYLIG EDVLPKQSLL YQKYEVLNEL | |
| | NNVRINNECL GTDQKQRLIR EVFERHSSVT IKQVADNLVA HGDFARRPEI | |
| | RGLADEKRFL SSLSTYHQLK EILHEAIDDP TKLLDIENII TWSTVFEDHT | |
| | IFETKLAEIE WLDPKKINEL SGIRYRGWGQ FSRKLLDGLK LGNGHTVIQE | |
| | LMLSNHNLMQ ILADETLKET MTELNQDKLK TDDIEDVIND AYTSPSNKKA | |
| | LRQVLRVVED IKHAANGQDP SWLFIETADG TGTAGKRTQS RQKQIQTVYA | |
| | NAAQELIDSA VRGELEDKIA DKASFTDRLV LYFMQGGRDI YTGAPLNIDQ | |
| | LSHYDIDHIL PQSLIKDDSL DNRVLVNATI NREKNNVFAS TLFAGKMKAT | |
| | WRKWHEAGLI SGRKLRNLML RPDEIDKFAK GFVARQLVET RQIIKLTEQI | |
| | AAAQYPNTKI IAVKAGLSHQ LREELDFPKN RDVNHYHHAF DAFLAARIGT | |
| | YLLKRYPKLA PFFTYGEFAK VDVKKFREFN FIGALTHAKK NIIAKDTGEI | |
| | VWDKERDIRE LDRIYNFKRM LITHEVYFET ADLFKQTIYA AKDSKERGGS | |
| | KQLIPKKQGY PTQVYGGYTQ ESGSYNALVR VAEADTTAYQ VIKISAQNAS | |
| | KIASANLKSR EKGKQLLNEI VVKQLAKRRK NWKPSANSFK IVIPRFGMGT | |
| | LFQNAKYGLF MVNSDTYYRN YQELWLSREN QKLLKKLFSI KYEKTQMNHD | |
| | ALQVYKAIID QVEKFFKLYD INQFRAKLSD AIERFEKLPI NTDGNKIGKT | |
| | ETLRQILIGL QANGTRSNVK NLGIKTDLGL LQVGSGIKLD KDTQIVYQSP | |
| | SGLFKRRIPL ADL | |
| Enterococcus | MYSIGLDLGI SSVGWSVIDE RTGNVIDLGV RLFSAKNSEK NLERRTNRGG | (SEQ |
| faecalis | RRLIRRKTNR LKDAKKILAA VGFYEDKSLK NSCPYQLRVK GLTEPLSRGE | ID NO: |
| TX0012 | IYKVTLHILK KRGISYLDEV DTEAAKESQD YKEQVRKNAQ LLTKYTPGQI | 105) |
| WP_ | QLQRLKENNR VKTGINAQGN YQLNVFKVSA YANELATILK TQQAFYPNEL | |
| 002408901.1 | TDDWIALFVQ PGIAEEAGLI YRKRPYYHGP GNEANNSPYG RWSDFQKTGE | |
| EFT93846.1 | PATNIFDKLI GKDFQGELRA SGLSLSAQQY NLLNDLTNLK IDGEVPLSSE | |
| | QKEYILTELM TKEFTRFGVN DVVKLLGVKK ERLSGWRLDK KGKPEIHTLK | |
| | GYRNWRKIFA EAGIDLATLP TETIDCLAKV LTLNTEREGI ENTLAFELPE | |
| | LSESVKLLVL DRYKELSQSI STQSWHRFSL KTLHLLIPEL MNATSEQNTL | |
| | LEQFQLKSDV RKRYSEYKKL PTKDVLAEIY NPTVNKTVSQ AFKVIDALLV | |
| | KYGKEQIRYI TIEMPRDDNE EDEKKRIKEL HAKNSQRKND SQSYFMQKSG | |
| | WSQEKFQTTI QKNRRFLAKL LYYYEQDGIC AYTGLPISPE LLVSDSTEID | |
| | HIIPISISLD DSINNKVLVL SKANQVKGQQ TPYDAWMDGS FKKINGKFSN | |
| | WDDYQKWVES RHFSHKKENN LLETRNIFDS EQVEKFLARN LNDTRYASRL | |
| | VLNTLQSFFT NQETKVRVVN GSFTHTLRKK WGADLDKTRE THHHHAVDAT | |
| | LCAVTSFVKV SRYHYAVKEE TGEKVMREID FETGEIVNEM SYWEFKKSKK | |
| | YERKTYQVKW PNFREQLKPV NLHPRIKFSH QVDRKANRKL SDATIYSVRE | |
| | KTEVKTLKSG KQKITTDEYT IGKIKDIYTL DGWEAFKKKQ DKLLMKDLDE | |
| | KTYERLLSIA ETTPDFQEVE EKNGKVKRVK RSPFAVYCEE NDIPAIQKYA | |
| | KKNNGPLIRS LKYYDGKLNK HINITKDSQG RPVEKTKNGR KVTLQSLKPY | |
| | RYDIYQDLET KAYYTVQLYY SDLRFVEGKY GITEKEYMKK VAEQTKGQWV | |
| | RFCFSLQKND GLEIEWKDSQ RYDVRFYNFQ SANSINFKGL EQEMMPAENQ | |
| | FKQKPYNNGA INLNIAKYGK EGKKLRKFNT DILGKKHYLF YEKEPKNIIK | |
| Candidatus | MRRLGLDLGT NSIGWCLLDL GDDGEPVSIF RTGARIFSDG RDPKSLGSLK | (SEQ |
| Puniceispirillum | ATRREARLTR RRRDRFIQRQ KNLINALVKY GLMPADEIQR QALAYKDPYP | ID NO: |
| marinum | IRKKALDEAI DPYEMGRAIF HINQRRGFKS NRKSADNEAG VVKQSIADLE | 106) |
| IMCC1322 | MKLGEAGART IGEFLADRQA TNDTVRARRL SGTNALYEFY PDRYMLEQEF | |
| WP_ | DTLWAKQAAF NPSLYIEAAR ERLKEIVFFQ RKLKPQEVGR CIFLSDEDRI | |
| 013047413.1 | SKALPSFQRF RIYQELSNLA WIDHDGVAHR ITASLALRDH LFDELEHKKK | |
| | LTFKAMRAIL RKQGVVDYPV GFNLESDNRD HLIGNLTSCI MRDAKKMIGS | |
| | AWDRLDEEEQ DSFILMLQDD QKGDDEVRSI LTQQYGLSDD VAEDCLDVRL | |
| | PDGHGSLSKK AIDRILPVLR DQGLIYYDAV KEAGLGEANL YDPYAALSDK | |
| | LDYYGKALAG HVMGASGKFE DSDEKRYGTI SNPTVHIALN QVRAVVNELI | |
| | RLHGKPDEVV IEIGRDLPMG ADGKRELERF QKEGRAKNER ARDELKKLGH | |
| | IDSRESRQKF QLWEQLAKEP VDRCCPFTGK MMSISDLFSD KVEIEHLLPF | |
| | SLTLDDSMAN KTVCFRQANR DKGNRAPFDA FGNSPAGYDW QEILGRSQNL | |
| | PYAKRWRFLP DAMKRFEADG GFLERQLNDT RYISRYTTEY ISTIIPKNKI | |
| | WVVTGRLTSL LRGFWGLNSI LRGHNTDDGT PAKKSRDDHR HHAIDAIVVG | |
| | MTSRGLLQKV SKAARRSEDL DLTRLFEGRI DPWDGFRDEV KKHIDAIIVS | |
| | HRPRKKSQGA LHNDTAYGIV EHAENGASTV VHRVPITSLG KQSDIEKVRD | |
| | PLIKSALLNE TAGLSGKSFE NAVQKWCADN SIKSLRIVET VSIIPITDKE | |
| | GVAYKGYKGD GNAYMDIYQD PTSSKWKGEI VSRFDANQKG FIPSWQSQFP | |
| | TARLIMRLRI NDLLKLQDGE IEEIYRVQRL SGSKILMAPH TEANVDARDR | |
| | DKNDTFKLTS KSPGKLQSAS ARKVHISPTG LIREG | |
| Oenococcus | MARDYSVGLD IGTSSVGWAA IDNKYHLIRA KSKNLIGVRL FDSAVTAEKR | (SEQ |
| kitaharae DSM | RGYRTTRRRL SRRHWRLRLL NDIFAGPLTD FGDENFLARL KYSWVHPQDQ | ID NO: |
| 17330 | SNQAHFAAGL LFDSKEQDKD FYRKYPTIYH LRLALMNDDQ KHDLREVYLA | 107) |
| EHN59352.1 | IHHLVKYRGH FLIEGDVKAD SAFDVHTFAD AIQRYAESNN SDENLLGKID | |
| | EKKLSAALTD KHGSKSQRAE TAETAFDILD LQSKKQIQAI LKSVVGNQAN | |
| | LMAIFGLDSS AISKDEQKNY KFSFDDADID EKIADSEALL SDTEFEFLCD | |
| | LKAAFDGLTL KMLLGDDKTV SAAMVRRFNE HQKDWEYIKS HIRNAKNAGN | |
| | GLYEKSKKFD GINAAYLALQ SDNEDDRKKA KKIFQDEISS ADIPDDVKAD | |
| | FLKKIDDDQF LPIQRTKNNG TIPHQLHRNE LEQIIEKQGI YYPFLKDTYQ | |
| | ENSHELNKIT ALINFRVPYY VGPLVEEEQK IADDGKNIPD PTNHWMVRKS | |
| | NDTITPWNLS QWVDLDKSGR RFIERLTGTD TYLIGEPTLP KNSLLYQKFD | |
| | VLQELNNIRV SGRRLDIRAK QDAFEHLFKV QKTVSATNLK DFLVQAGYIS | |
| | EDTQIEGLAD VNGKNFNNAL TTYNYLVSVL GREFVENPSN EELLEEITEL | |
| | QTVFEDKKVL RRQLDQLDGL SDHNREKLSR KHYTGWGRIS KKLLTTKIVQ | |
| | NADKIDNQTF DVPRMNQSII DTLYNTKMNL MEIINNAEDD FGVRAWIDKQ | |
| | NTTDGDEQDV YSLIDELAGP KEIKRGIVQS FRILDDITKA VGYAPKRVYL | |
| | EFARKTQESH LTNSRKNQLS TLLKNAGLSE LVTQVSQYDA AALQNDRLYL | |
| | YFLQQGKDMY SGEKLNLDNL SNYDIDHIIP QAYTKDNSLD NRVLVSNITN | |
| | RRKSDSSNYL PALIDKMRPF WSVLSKQGLL SKHKFANLTR TRDFDDMEKE | |
| | RFIARSLVET RQIIKNVASL IDSHFGGETK AVAIRSSLTA DMRRYVDIPK | |
| | NRDINDYHHA FDALLFSTVG QYTENSGLMK KGQLSDSAGN QYNRYIKEWI | |
| | HAARLNAQSQ RVNPFGFVVG SMRNAAPGKL NPETGEITPE ENADWSIADL | |
| | DYLHKVMNFR KITVTRRLKD QKGQLYDESR YPSVLHDAKS KASINFDKHK | |
| | PVDLYGGFSS AKPAYAALIK FKNKFRLVNV LRQWTYSDKN SEDYILEQIR | |
| | GKYPKAEMVL SHIPYGQLVK KDGALVTISS ATELHNFEQL WLPLADYKLI | |
| | NTLLKTKEDN LVDILHNRLD LPEMTIESAF YKAFDSILSF AFNRYALHQN | |
| | ALVKLQAHRD DFNALNYEDK QQTLERILDA LHASPASSDL KKINLSSGFG | |
| | RLFSPSHFTL ADTDEFIFQS VTGLFSTQKT VAQLYQETK | |
| Helicobacter | MIRTLGIDIG IASIGWAVIE GEYTDKGLEN KEIVASGVRV FTKAENPKNK | (SEQ |
| mustelae | ESLALPRTLA RSARRRNARK KGRIQQVKHY LSKALGLDLE CFVQGEKLAT | ID NO: |
| 12198 | LFQTSKDFLS PWELRERALY RVLDKEELAR VILHIAKRRG YDDITYGVED | 108) |
| WP_ | NDSGKIKKAI AENSKRIKEE QCKTIGEMMY KLYFQKSLNV RNKKESYNRC | |
| 013022389.1 | VGRSELREEL KTIFQIQQEL KSPWVNEELI YKLLGNPDAQ SKQEREGLIF | |
| | YQRPLKGFGD KIGKCSHIKK GENSPYRACK HAPSAEEFVA LTKSINFLKN | |
| | LTNRHGLCFS QEDMCVYLGK ILQEAQKNEK GLTYSKLKLL LDLPSDFEFL | |
| | GLDYSGKNPE KAVFLSLPST FKLNKITQDR KTQDKIANIL GANKDWEAIL | |
| | KELESLQLSK EQIQTIKDAK LNFSKHINLS LEALYHLLPL MREGKRYDEG | |
| | VEILQERGIF SKPQPKNRQL LPPLSELAKE ESYFDIPNPV LRRALSEFRK | |
| | WVNALLEKYG GFHYFHIELT RDVCKAKSAR MQLEKINKKN KSENDAASQL | |
| | LEVLGLPNTY NNRLKCKLWK QQEEYCLYSG EKITIDHLKD QRALQIDHAF | |
| | PLSRSLDDSQ SNKVLCLTSS NQEKSNKTPY EWLGSDEKKW DMYVGRVYSS | |
| | NFSPSKKRKL TQKNFKERNE EDFLARNLVD TGYIGRVTKE YIKHSLSFLP | |
| | LPDGKKEHIR IISGSMTSTM RSFWGVQEKN RDHHLHHAQD AIIIACIEPS | |
| | MIQKYTTYLK DKETHRLKSH QKAQILREGD HKLSLRWPMS NFKDKIQESI | |
| | QNIIPSHHVS HKVTGELHQE TVRTKEFYYQ AFGGEEGVKK ALKFGKIREI | |
| | NQGIVDNGAM VRVDIFKSKD KGKFYAVPIY TYDFAIGKLP NKAIVQGKKN | |
| | GIIKDWLEMD ENYEFCFSLF KNDCIKIQTK EMQEAVLAIY KSTNSAKATI | |
| | ELEHLSKYAL KNEDEEKMFT DTDKEKNKTM TRESCGIQGL KVFQKVKLSV | |
| | LGEVLEHKPR NRQNIALKTT PKHV | |
| Bradyrhizobium | MKRTSLRAYR LGVDLGANSL GWFVVWLDDH GQPEGLGPGG VRIFPDGRNP | (SEQ |
| sp. BTAi1 | QSKQSNAAGR RLARSARRRR DRYLQRRGKL MGLLVKHGLM PADEPARKRL | ID NO: |
| WP_ | ECLDPYGLRA KALDEVLPLH HVGRALFHLN QRRGLFANRA IEQGDKDASA | 109) |
| 012044026.1 | IKAAAGRLQT SMQACGARTL GEFLNRRHQL RATVRARSPV GGDVQARYEF | |
| | YPTRAMVDAE FEAIWAAQAP HHPTMTAEAH DTIREAIFSQ RAMKRPSIGK | |
| | CSLDPATSQD DVDGFRCAWS HPLAQRFRIW QDVRNLAVVE TGPTSSRLGK | |
| | EDQDKVARAL LQTDQLSFDE IRGLLGLPSD ARFNLESDRR DHLKGDATGA | |
| | ILSARRHFGP AWHDRSLDRQ IDIVALLESA LDEAAIIASL GTTHSLDEAA | |
| | AQRALSALLP DGYCRLGLRA IKRVLPLMEA GRTYAEAASA AGYDHALLPG | |
| | GKLSPTGYLP YYGQWLQNDV VGSDDERDTN ERRWGRLPNP TVHIGIGQLR | |
| | RVVNELIRWH GPPAEITVEL TRDLKLSPRR LAELEREQAE NQRKNDKRTS | |
| | LLRKLGLPAS THNLLKLRLW DEQGDVASEC PYTGEAIGLE RLVSDDVDID | |
| | HLIPFSISWD DSAANKVVCM RYANREKGNR TPFEAFGHRQ GRPYDWADIA | |
| | ERAARLPRGK RWRFGPGARA QFEELGDFQA RLLNETSWLA RVAKQYLAAV | |
| | THPHRIHVLP GRLTALLRAT WELNDLLPGS DDRAAKSRKD HRHHAIDALV | |
| | AALTDQALLR RMANAHDDTR RKIEVLLPWP TFRIDLETRL KAMLVSHKPD | |
| | HGLQARLHED TAYGTVEHPE TEDGANLVYR KTFVDISEKE IDRIRDRRLR | |
| | DLVRAHVAGE RQQGKTLKAA VLSFAQRRDI AGHPNGIRHV RLTKSIKPDY | |
| | LVPIRDKAGR IYKSYNAGEN AFVDILQAES GRWIARATTV FQANQANESH | |
| | DAPAAQPIMR VFKGDMLRID HAGAEKFVKI VRLSPSNNLL YLVEHHQAGV | |
| | FQTRHDDPED SFRWLFASFD KLREWNAELV RIDTLGQPWR RKRGLETGSE | |
| | DATRIGWTRP KKWP | |
| Acidaminococcus | MGKMYYLGLD IGTNSVGYAV TDPSYHLLKF KGEPMWGAHV FAAGNQSAER | (SEQ |
| sp. D21 | RSFRTSRRRL DRRQQRVKLV QEIFAPVISP IDPRFFIRLH ESALWRDDVA | ID NO: |
| WP_00901621 | ETDKHIFFND PTYTDKEYYS DYPTIHHLIV DLMESSEKHD PRLVYLAVAW | 110) |
| 9.1 | LVAHRGHFLN EVDKDNIGDV LSFDAFYPEF LAFLSDNGVS PWVCESKALQ | |
| | ATLLSRNSVN DKYKALKSLI FGSQKPEDNF DANISEDGLI QLLAGKKVKV | |
| | NKLFPQESND ASFTLNDKED AIEEILGTLT PDECEWIAHI RRLFDWAIMK | |
| | HALKDGRTIS ESKVKLYEQH HHDLTQLKYF VKTYLAKEYD DIFRNVDSET | |
| | TKNYVAYSYH VKEVKGTLPK NKATQEEFCK YVLGKVKNIE CSEADKVDFD | |
| | EMIQRLTDNS FMPKQVSGEN RVIPYQLYYY ELKTILNKAA SYLPFLTQCG | |
| | KDAISNQDKL LSIMTFRIPY FVGPLRKDNS EHAWLERKAG KIYPWNFNDK | |
| | VDLDKSEEAF IRRMTNTCTY YPGEDVLPLD SLIYEKFMIL NEINNIRIDG | |
| | YPISVDVKQQ VFGLFEKKRR VTVKDIQNLL LSLGALDKHG KLTGIDTTIH | |
| | SNYNTYHHFK SLMERGVLTR DDVERIVERM TYSDDTKRVR LWLNNNYGTL | |
| | TADDVKHISR LRKHDFGRLS KMFLTGLKGV HKETGERASI LDFMWNTNDN | |
| | LMQLLSECYT FSDEITKLQE AYYAKAQLSL NDFLDSMYIS NAVKRPIYRT | |
| | LAVVNDIRKA CGTAPKRIFI EMARDGESKK KRSVTRREQI KNLYRSIRKD | |
| | FQQEVDFLEK ILENKSDGQL QSDALYLYFA QLGRDMYTGD PIKLEHIKDQ | |
| | SFYNIDHIYP QSMVKDDSLD NKVLVQSEIN GEKSSRYPLD AAIRNKMKPL | |
| | WDAYYNHGLI SLKKYQRLTR STPFTDDEKW DFINRQLVET RQSTKALAIL | |
| | LKRKFPDTEI VYSKAGLSSD FRHEFGLVKS RNINDLHHAK DAFLAIVTGN | |
| | VYHERFNRRW FMVNQPYSVK TKTLFTHSIK NGNFVAWNGE EDLGRIVKML | |
| | KQNKNTIHFT RFSFDRKEGL FDIQPLKAST GLVPRKAGLD VVKYGGYDKS | |
| | TAAYYLLVRF TLEDKKTQHK LMMIPVEGLY KARIDHDKEF LTDYAQTTIS | |
| | EILQKDKQKV INIMFPMGTR HIKLNSMISI DGFYLSIGGK SSKGKSVLCH | |
| | AMVPLIVPHK IECYIKAMES FARKFKENNK LRIVEKFDKI TVEDNLNLYE | |
| | LFLQKLQHNP YNKFFSTQFD VLTNGRSTFT KLSPEEQVQT LLNILSIFKT | |
| | CRSSGCDLKS INGSAQAARI MISADLTGLS KKYSDIRLVE QSASGLFVSK | |
| | SQNLLEYL | |
| Methylosinus | MRVLGLDAGI ASLGWALIEI EESNRGELSQ GTIIGAGTWM FDAPEEKTQA | (SEQ |
| trichosporium | GAKLKSEQRR TFRGQRRVVR RRRQRMNEVR RILHSHGLLP SSDRDALKQP | ID NO: |
| OB3b | GLDPWRIRAE ALDRLLGPVE LAVALGHIAR HRGFKSNSKG AKTNDPADDT | |
| WP_00361103 | SKMKRAVNET REKLARFGSA AKMLVEDESF VLRQTPTKNG ASEIVRRFRN | |
| 4.1 | REGDYSRSLL RDDLAAEMRA LFTAQARFQS AIATADLQTA FTKAAFFQRP | 111) |
| | LQDSEKLVGP CPFEVDEKRA PKRGYSFELF RFLSRLNHVT LRDGKQERTL | |
| | TRDELALAAA DFGAAAKVSF TALRKKLKLP ETTVFVGVKA DEESKLDVVA | |
| | RSGKAAEGTA RLRSVIVDAL GELAWGALLC SPEKLDKIAE VISFRSDIGR | |
| | ISEGLAQAGC NAPLVDALTA AASDGRFDPF TGAGHISSKA ARNILSGLRQ | |
| | GMTYDKACCA ADYDHTASRE RGAFDVGGHG REALKRILQE ERISRELVGS | |
| | PTARKALIES IKQVKAIVER YGVPDRIHVE LARDVGKSIE EREEITRGIE | |
| | KRNRQKDKLR GLFEKEVGRP PQDGARGKEE LLRFELWSEQ MGRCLYTDDY | |
| | ISPSQLVATD DAVQVDHILP WSRFADDSYA NKTLCMAKAN QDKKGRTPYE | |
| | WFKAEKTDTE WDAFIVRVEA LADMKGFKKR NYKLRNAEEA AAKFRNRNLN | |
| | DTRWACRLLA EALKQLYPKG EKDKDGKERR RVFSRPGALT DRLRRAWGLQ | |
| | WMKKSTKGDR IPDDRHHALD AIVIAATTES LLQRATREVQ EIEDKGLHYD | |
| | LVKNVTPPWP GFREQAVEAV EKVFVARAER RRARGKAHDA TIRHIAVREG | |
| | EQRVYERRKV AELKLADLDR VKDAERNARL IEKLRNWIEA GSPKDDPPLS | |
| | PKGDPIFKVR LVTKSKVNIA LDTGNPKRPG TVDRGEMARV DVFRKASKKG | |
| | KYEYYLVPIY PHDIATMKTP PIRAVQAYKP EDEWPEMDSS YEFCWSLVPM | |
| | TYLQVISSKG EIFEGYYRGM NRSVGAIQLS AHSNSSDVVQ GIGARTLTEF | |
| | KKFNVDRFGR KHEVERELRT WRGETWRGKA YI | |
| Actinomyces | MDNKNYRIGI DVGLNSIGFC AVEVDQHDTP LGFLNLSVYR HDAGIDPNGK | (SEQ |
| coleocanis | KTNTTRLAMS GVARRTRRLF RKRKRRLAAL DRFIEAQGWT LPDHADYKDP | ID NO: |
| DSM 15436 | YTPWLVRAEL AQTPIRDEND LHEKLAIAVR HIARHRGWRS PWVPVRSLHV | 112) |
| WP_ | EQPPSDQYLA LKERVEAKTL LQMPEGATPA EMVVALDLSV DVNLRPKNRE | |
| 006546479.1 | KTDTRPENKK PGFLGGKLMQ SDNANELRKI AKIQGLDDAL LRELIELVFA | |
| | ADSPKGASGE LVGYDVLPGQ HGKRRAEKAH PAFQRYRIAS IVSNLRIRHL | |
| | GSGADERLDV ETQKRVFEYL LNAKPTADIT WSDVAEEIGV ERNLLMGTAT | |
| | QTADGERASA KPPVDVTNVA FATCKIKPLK EWWLNADYEA RCVMVSALSH | |
| | AEKLTEGTAA EVEVAEFLQN LSDEDNEKLD SFSLPIGRAA YSVDSLERLT | |
| | KRMIENGEDL FEARVNEFGV SEDWRPPAEP IGARVGNPAV DRVLKAVNRY | |
| | LMAAEAEWGA PLSVNIEHVR EGFISKRQAV EIDRENQKRY QRNQAVRSQI | |
| | ADHINATSGV RGSDVTRYLA IQRQNGECLY CGTAITFVNS EMDHIVPRAG | |
| | LGSTNTRDNL VATCERCNKS KSNKPFAVWA AECGIPGVSV AEALKRVDFW | |
| | IADGFASSKE HRELQKGVKD RLKRKVSDPE IDNRSMESVA WMARELAHRV | |
| | QYYFDEKHTG TKVRVFRGSL TSAARKASGF ESRVNFIGGN GKTRLDRRHH | |
| | AMDAATVAML RNSVAKTLVL RGNIRASERA IGAAETWKSF RGENVADRQI | |
| | FESWSENMRV LVEKFNLALY NDEVSIFSSL RLQLGNGKAH DDTITKLQMH | |
| | KVGDAWSLTE IDRASTPALW CALTRQPDFT WKDGLPANED RTIIVNGTHY | |
| | GPLDKVGIFG KAAASLLVRG GSVDIGSAIH HARIYRIAGK KPTYGMVRVF | |
| | APDLLRYRNE DLFNVELPPQ SVSMRYAEPK VREAIREGKA EYLGWLVVGD | |
| | ELLLDLSSET SGQIAELQQD FPGTTHWTVA GFFSPSRLRL RPVYLAQEGL | |
| | GEDVSEGSKS IIAGQGWRPA VNKVFGSAMP EVIRRDGLGR KRRFSYSGLP | |
| | VSWQG | |
| Caenispirillum | MPVLSPLSPN AAQGRRRWSL ALDIGEGSIG WAVAEVDAEG RVLQLTGTGV | (SEQ |
| salinarum AK4 | TLFPSAWSNE NGTYVAHGAA DRAVRGQQQR HDSRRRRLAG LARLCAPVLE | ID NO: |
| WP_009541330.1 | RSPEDLKDLT RTPPKADPRA IFFLRADAAR RPLDGPELFR VLHHMAAHRG | 113) |
| | IRLAELQEVD PPPESDADDA APAATEDEDG TRRAAADERA FRRLMAEHMH | |
| | RHGTQPTCGE IMAGRLRETP AGAQPVTRAR DGLRVGGGVA VPTRALIEQE | |
| | FDAIRAIQAP RHPDLPWDSL RRLVLDQAPI AVPPATPCLF LEELRRRGET | |
| | FQGRTITREA IDRGLTVDPL IQALRIRETV GNLRLHERIT EPDGRQRYVP | |
| | RAMPELGLSH GELTAPERDT LVRALMHDPD GLAAKDGRIP YTRLRKLIGY | |
| | DNSPVCFAQE RDTSGGGITV NPTDPLMARW IDGWVDLPLK ARSLYVRDVV | |
| | ARGADSAALA RLLAEGAHGV PPVAAAAVPA ATAAILESDI MQPGRYSVCP | |
| | WAAEAILDAW ANAPTEGFYD VTRGLFGFAP GEIVLEDLRR ARGALLAHLP | |
| | RTMAAARTPN RAAQQRGPLP AYESVIPSQL ITSLRRAHKG RAADWSAADP | |
| | EERNPFLRTW TGNAATDHIL NQVRKTANEV ITKYGNRRGW DPLPSRITVE | |
| | LAREAKHGVI RRNEIAKENR ENEGRRKKES AALDTFCQDN TVSWQAGGLP | |
| | KERAALRLRL AQRQEFFCPY CAERPKLRAT DLFSPAETEI DHVIERRMGG | |
| | DGPDNLVLAH KDCNNAKGKK TPHEHAGDLL DSPALAALWQ GWRKENADRL | |
| | KGKGHKARTP REDKDFMDRV GWRFEEDARA KAEENQERRG RRMLHDTARA | |
| | TRLARLYLAA AVMPEDPAEI GAPPVETPPS PEDPTGYTAI YRTISRVQPV | |
| | NGSVTHMLRQ RLLQRDKNRD YQTHHAEDAC LLLLAGPAVV QAFNTEAAQH | |
| | GADAPDDRPV DLMPTSDAYH QQRRARALGR VPLATVDAAL ADIVMPESDR | |
| | QDPETGRVHW RLTRAGRGLK RRIDDLTRNC VILSRPRRPS ETGTPGALHN | |
| | ATHYGRREIT VDGRTDTVVT QRMNARDLVA LLDNAKIVPA ARLDAAAPGD | |
| | TILKEICTEI ADRHDRVVDP EGTHARRWIS ARLAALVPAH AEAVARDIAE | |
| | LADLDALADA DRTPEQEARR SALRQSPYLG RAISAKKADG RARAREQEIL | |
| | TRALLDPHWG PRGLRHLIMR EARAPSLVRI RANKTDAFGR PVPDAAVWVK | |
| | TDGNAVSQLW RLTSVVTDDG RRIPLPKPIE KRIEISNLEY ARLNGLDEGA | |
| | GVTGNNAPPR PLRQDIDRLT PLWRDHGTAP GGYLGTAVGE LEDKARSALR | |
| | GKAMRQTLTD AGITAEAGWR LDSEGAVCDL EVAKGDTVKK DGKTYKVGVI | |
| | TQGIFGMPVD AAGSAPRTPE DCEKFEEQYG IKPWKAKGIP LA | |
| Coriobacterium | MKLRGIEDDY SIGLDMGTSS VGWAVTDERG TLAHFKRKPT WGSRLFREAQ | (SEQ |
| glomerans | TAAVARMPRG QRRRYVRRRW RLDLLQKLFE QQMEQADPDF FIRLRQSRLL | ID NO: |
| PW2 | RDDRAEEHAD YRWPLFNDCK FTERDYYQRF PTIYHVRSWL METDEQADIR | 114) |
| WP_ | LIYLALHNIV KHRGNFLREG QSLSAKSARP DEALNHLRET LRVWSSERGF | |
| 013709575.1 | ECSIADNGSI LAMLTHPDLS PSDRRKKIAP LFDVKSDDAA ADKKLGIALA | |
| | GAVIGLKTEF KNIFGDFPCE DSSIYLSNDE AVDAVRSACP DDCAELFDRL | |
| | CEVYSAYVLQ GLLSYAPGQT ISANMVEKYR RYGEDLALLK KLVKIYAPDQ | |
| | YRMFFSGATY PGTGIYDAAQ ARGYTKYNLG PKKSEYKPSE SMQYDDFRKA | |
| | VEKLFAKTDA RADERYRMMM DRFDKQQFLR RLKTSDNGSI YHQLHLEELK | |
| | AIVENQGRFY PFLKRDADKL VSLVSFRIPY YVGPLSTRNA RTDQHGENRF | |
| | AWSERKPGMQ DEPIFPWNWE SIIDRSKSAE KFILRMTGMC TYLQQEPVLP | |
| | KSSLLYEEFC VLNELNGAHW SIDGDDEHRF DAADREGIIE ELFRRKRTVS | |
| | YGDVAGWMER ERNQIGAHVC GGQGEKGFES KLGSYIFFCK DVFKVERLEQ | |
| | SDYPMIERII LWNTLFEDRK ILSQRLKEEY GSRLSAEQIK TICKKRFTGW | |
| | GRLSEKFLTG ITVQVDEDSV SIMDVLREGC PVSGKRGRAM VMMEILRDEE | |
| | LGFQKKVDDF NRAFFAENAQ ALGVNELPGS PAVRRSLNQS IRIVDEIASI | |
| | AGKAPANIFI EVTRDEDPKK KGRRTKRRYN DLKDALEAFK KEDPELWREL | |
| | CETAPNDMDE RLSLYFMQRG KCLYSGRAID IHQLSNAGIY EVDHIIPRTY | |
| | VKDDSLENKA LVYREENQRK TDMLLIDPEI RRRMSGYWRM LHEAKLIGDK | |
| | KFRNLLRSRI DDKALKGFIA RQLVETGQMV KLVRSLLEAR YPETNIISVK | |
| | ASISHDLRTA AELVKCREAN DFHHAHDAFL ACRVGLFIQK RHPCVYENPI | |
| | GLSQWVRNYV RQQADIFKRC RTIPGSSGFI VNSFMTSGFD KETGEIFKDD | |
| | WDAEAEVEGI RRSLNFRQCF ISRMPFEDHG VFWDATIYSP RAKKTAALPL | |
| | KQGLNPSRYG SFSREQFAYF FIYKARNPRK EQTLFEFAQV PVRLSAQIRQ | |
| | DENALERYAR ELAKDQGLEF IRIERSKILK NQLIEIDGDR LCITGKEEVR | |
| | NACELAFAQD EMRVIRMLVS EKPVSRECVI SLFNRILLHG DQASRRLSKQ | |
| | LKLALLSEAF SEASDNVQRN VVLGLIAIFN GSTNMVNLSD IGGSKFAGNV | |
| | RIKYKKELAS PKVNVHLIDQ SVTGMFERRT KIGL | |

In some embodiments, prime editors utilized herein comprise CRISPR-Cas system enzymes other than type II enzymes. In certain embodiments, prime editors comprise type V or type VI CRISPR-Cas system enzymes. It will be appreciated that certain CRISPR enzymes exhibit promiscuous ssDNA cleavage activity and appropriate precautions should be considered. In certain embodiments, prime editors comprise a nickase or a dead CRISPR with nuclease function comprised in a different component.

In various embodiments, the nucleic acid programmable DNA binding proteins utilized herein include, without limitation, Cas9 (e.g., dCas9 and nCas9), Cas12a (Cpf1), Cas12b1 (C2c1), Cas12b2, Cas12c (C2c3), Cas12d (CasY), Cas12e (CasX), C2c4, C2c5, C2c8, C2c9, C2c10, Cas13a (C2c2), Cas13b (C2c6), Cas13c (C2c7), Cas13d, and Argonaute. Cas-equivalents further include those described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018, the contents of which are incorporated herein by reference. One example of a nucleic acid programmable DNA-binding protein that has different PAM specificity than Cas9 is Clustered Regularly Interspaced Short Palindromic Repeats from Prevotella and Francisella 1 (i.e, Cas12a (Cpf1)). Similar to Cas9, Cas12a (Cpf1) is also a Class 2 CRISPR effector, but it is a member of type V subgroup of enzymes, rather than the type II subgroup. It has been shown that Cas12a (Cpf1) mediates robust DNA interference with features distinct from Cas9. Cas12a (Cpf1) is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

### 6.3. Type V CRISPR Proteins

In some embodiments, prime editors used herein comprise the type V CRISPR family includes Francisella novicida U112 Cpf1 (FnCpf1) also known as FnCas12a. FnCpf1 adopts a bilobed architecture with the two lobes connected by the wedge (WED) domain. The N-terminal REC lobe consists of two a-helical domains (REC1 and REC2) that have been shown to coordinate the crRNA-target DNA heteroduplex. The C-terminal NUC lobe consists of the C-terminal RuvC and Nuc domains involved in target cleavage, the arginine-rich bridge helix (BH), and the PAM-interacting (PI) domain. The repeat-derived segment of the crRNA forms a pseudoknot stabilized by intra-molecular base-pairing and hydrogen-bonding interactions. The pseudoknot is coordinated by residues from the WED, RuvC, and REC2 domains, as well as by two hydrated magnesium cations. Notably, nucleotides 1-5 of the crRNA are ordered in the central cavity of FnCas12a and adopt an A-form-like helical conformation. Conformational ordering of the seed sequence is facilitated by multiple interactions between the ribose and phosphate moieties of the crRNA backbone and FnCpf1 residues in the WED and REC1 domains. These include residues Thr16, Lys595, His804, and His881 from the WED domain and residues Tyr47, Lys51, Phe182, and Arg186 from the REC1 domain. The structure of the FnCas12a-crRNA complex further reveals that the bases of the seed sequence are solvent exposed and poised for hybridization with target DNA. Structural aspects of FnCpf1 are described by Swarts et al., Structural Basis for Guide RNA Processing and Seed-Dependent DNA Targeting by CRISPR-Cas12a, Molecular Cell 66, 221-233, Apr. 20, 2017.

Pre-crRNA processing: Essential residues for crRNA processing include His843, Lys852, and Lys869. Structural observations are consistent with an acid-base catalytic mechanism in which Lys869 acts as the general base catalyst to deprotonate the attacking 2′-hydroxyl group of U(-19), while His843 acts as a general acid to protonate the 5′-oxygen leaving group of A(-18). In turn, the side chain of Lys852 is involved in charge stabilization of the transition state. Collectively, these interactions facilitate the intra-molecular attack of the 20-hydroxyl group of U(-19) on the scissile phosphate and promote the formation of the 2′,3′-cyclic phosphate product.

R-loop formation: The crRNA-target DNA strand heteroduplex is enclosed in the central cavity formed by the REC and NUC lobes and interacts extensively with the REC1 and REC2 domains. The PAM-containing DNA duplex comprises target strand nucleotides dT0-dT8 and non-target strand nucleotides dA(8)*-dA0* and is contacted by the PI, WED, and REC1 domains. The 5′-TTN-3′ PAM is recognized in FnCas12a by a mechanism combining the shape-specific recognition of a narrowed minor groove, with base-specific recognition of the PAM bases by two invariant residues, Lys671 and Lys613. Directly downstream of the PAM, the duplex of the target DNA is disrupted by the side chain of residue Lys667, which is inserted between the DNA strands and forms a cation-π stacking interaction with the dA0-dT0* base pair. The phosphate group linking target strand residues dT(-1) and dT0 is coordinated by hydrogen-bonding interactions with the side chain of Lys823 and the backbone amide of Gly826. Target strand residue dT(-1) bends away from residue TO, allowing the target strand to interact with the seed sequence of the crRNA. The non-target strand nucleotides dT1*-dT5* interact with the Arg692-Ser702 loop in FnCas12a through hydrogen-bonding and ionic interactions between backbone phosphate groups and side chains of Arg692, Asn700, Ser702, and Gln704, as well as main-chain amide groups of Lys699, Asn700, and Ser702. Alanine substitution of Q704 or replacement of residues Thr698-Ser702 in FnCas12a with the sequence Ala-Gly3 (SEQ ID NO: 115) substantially reduced DNA cleavage activity, suggesting that these residues contribute to R-loop formation by stabilizing the displaced conformation of the nontarget DNA strand.

In the FnCas12a R-loop complex, the crRNA-target strand heteroduplex is terminated by a stacking interaction with a conserved aromatic residue (Tyr410). This prevents base pairing between the crRNA and the target strand beyond nucleotides U20 and dA(-20), respectively. Beyond this point, the target DNA strand nucleotides re-engage the non-target DNA strand, forming a PAM-distal DNA duplex comprising nucleotides dC(-21)-dA(-27) and dG21*-dT27*, respectively. The duplex is confined between the REC2 and Nuc domains at the end of the central channel formed by the REC and NUC lobes.

Target DNA cleavage: FnCpf1 can independently accommodate both the target and non-target DNA strands in the catalytic pocket of the RuvC domain. The RuvC active site contains three catalytic residues (D917, E1006, and D1255). Structural observations suggest that both the target and non-target DNA strands are cleaved by the same catalytic mechanism in a single active site in Cpf1/Cas12a enzymes.

Another type V CRISPR is AsCpf1 from Acidaminococcus sp BV3L6 (Yamano et al., Crystal structure of Cpf1 in complex with guide RNA and target DNA, Cell 165, 949-962, May 5, 2016)

In certain embodiments, the nuclease comprises a Cas12f effector. Small CRISPR-associated effector proteins belonging to the type V-F subtype have been identified through the mining of sequence databases and members classified into Cas12f1 (Cas14a and type V-U3), Cas12f2 (Cas14b) and Cas12f3 (Cas14c, type V-U2 and U4). (See, e.g., Karvelis et al., PAM recognition by miniature CRISPR-Cas12f nucleases triggers programmable double-stranded DNA target cleavage. Nucleic Acids Research, 21 May 2020, 48(9), 5016-23 doi.org/10.1093/nar/gkaa208). Xu et al. described development of a 529 amino acid Cas12f-based system for mammalian genome engineering through multiple rounds of iterative protein engineering and screening. (Xu, X. et al., Engineered Miniature CRISPR-Cas System for Mammalian Genome Regulation and Editing. Molecular Cell, Oct. 21, 2021, 81(20): 4333-45, doi.org/10.1016/j.molcel.2021.08.008).

Exemplary CRISPR-Cas proteins and enzymes used in the prime editors herein include the following without limitation.

TABLE 5
Cas12a orthologs

| | | |
|---|---|---|
| KKP36646_ | MSNFFKNFTN LYELSKTLRF ELKPVGDTLT NMKDHLEYDE KLQTFLKDQN | (SEQ |
| (modified) | IDDAYQALKP QFDEIHEEFI TDSLESKKAK EIDFSEYLDL FQEKKELNDS | ID NO: |
| hypothetical | EKKLRNKIGE TFNKAGEKWK KEKYPQYEWK KGSKIANGAD ILSCQDMLQF | 116) |
| protein | IKYKNPEDEK IKNYIDDTLK GFFTYFGGFN QNRANYYETK KEASTAVATR | |
| UR27_C0015G0 | IVHENLPKFC DNVIQFKHII KRKKDGTVEK TERKTEYLNA YQYLKNNNKI | |
| 004 [Candidatus | TQIKDAETEK MIESTPIAEK IFDVYYFSSC LSQKQIEEYN RIIGHYNLLI | |
| Peregrinibacteria | NLYNQAKRSE GKHLSANEKK YKDLPKFKTL YKQIGCGKKK DLFYTIKCDT | |
| bacterium | EEEANKSRNE GKESHSVEEI INKAQEAINK YFKSNNDCEN INTVPDFINY | |
| GW2011_GWA2 | ILTKENYEGV YWSKAAMNTI SDKYFANYHD LQDRLKEAKV FQKADKKSED | |
| _33_10] | DIKIPEAIEL SGLFGVLDSL ADWQTTLFKS SILSNEDKLK IITDSQTPSE | |
| | ALLKMIFNDI EKNMESFLKE TNDIITLKKY KGNKEGTEKI KQWFDYTLAI | |
| | NRMLKYFLVK ENKIKGNSLD TNISEALKTL IYSDDAEWFK WYDALRNYLT | |
| | QKPQDEAKEN KLKLNFDNPS LAGGWDVNKE CSNFCVILKD KNEKKYLAIM | |
| | KKGENTLFQK EWTEGRGKNL TKKSNPLFEI NNCEILSKME YDFWADVSKM | |
| | IPKCSTQLKA VVNHFKQSDN EFIFPIGYKV TSGEKFREEC KISKQDFELN | |
| | NKVFNKNELS VTAMRYDLSS TQEKQYIKAF QKEYWELLFK QEKRDTKLTN | |
| | NEIFNEWINF CNKKYSELLS WERKYKDALT NWINFCKYFL SKYPKTTLFN | |
| | YSFKESENYN SLDEFYRDVD ICSYKLNINT TINKSILDRL VEEGKLYLFE | |
| | IKNQDSNDGK SIGHKNNLHT IYWNAIFENF DNRPKLNGEA EIFYRKAISK | |
| | DKLGIVKGKK TKNGTEIIKN YRFSKEKFIL HVPITLNFCS NNEYVNDIVN | |
| | TKFYNFSNLH FLGIDRGEKH LAYYSLVNKN GEIVDQGTLN LPFTDKDGNQ | |
| | RSIKKEKYFY NKQEDKWEAK EVDCWNYNDL LDAMASNRDM ARKNWQRIGT | |
| | IKEAKNGYVS LVIRKIADLA VNNERPAFIV LEDLNTGFKR SRQKIDKSVY | |
| | QKFELALAKK LNFLVDKNAK RDEIGSPTKA LQLTPPVNNY GDIENKKQAG | |
| | IMLYTRANYT SQTDPATGWR KTIYLKAGPE ETTYKKDGKI KNKSVKDQII | |
| | ETFTDIGFDG KDYYFEYDKG EFVDEKTGEI KPKKWRLYSG ENGKSLDRFR | |
| | GEREKDKYEW KIDKIDIVKI LDDLFVNFDK NISLLKQLKE GVELTRNNEH | |
| | GTGESLRFAI NLIQQIRNTG NNERDNDFIL SPVRDENGKH FDSREYWDKE | |
| | TKGEKISMPS SGDANGAFNI ARKGIIMNAH ILANSDSKDL SLFVSDEEWD | |
| | LHLNNKTEWK KQLNIFSSRK AMAKRKK | |
| KKR91555_ | MLFFMSTDIT NKPREKGVFD NFTNLYEFSK TLTFGLIPLK WDDNKKMIVE | (SEQ |
| (modified) | DEDFSVLRKY GVIEEDKRIA ESIKIAKFYL NILHRELIGK VLGSLKFEKK | ID NO: |
| hypothetical | NLENYDRLLG EIEKNNKNEN ISEDKKKEIR KNFKKELSIA QDILLKKVGE | 117) |
| protein | VFESNGSGIL SSKNCLDELT KRFTRQEVDK LRRENKDIGV EYPDVAYREK | |
| UU43_ | DGKEETKSFF AMDVGYLDDF HKNRKQLYSV KGKKNSLGRR ILDNFEIFCK | |
| C0004G0003 | NKKLYEKYKN LDIDFSEIER NFNLTLEKVF DFDNYNERLT QEGLDEYAKI | |
| [Parcubacteria | LGGESNKQER TANIHGLNQI INLYIQKKQS EQKAEQKETG KKKIKFNKKD | |
| (Falkowbacteria) | YPTFTCLQKQ ILSQVFRKEI IIESDRDLIR ELKFFVEESK EKVDKARGII | |
| bacterium | EFLLNHEEND IDLAMVYLPK SKINSFVYKV FKEPQDFLSV FQDGASNLDF | |
| GW2011_GWA2 | VSFDKIKTHL ENNKLTYKIF FKTLIKENHD FESFLILLQQ EIDLLIDGGE | |
| _41_14] | TVTLGGKKES ITSLDEKKNR LKEKLGWFEG KVRENEKMKD EEEGEFCSTV | |
| | LAYSQAVLNI TKRAEIFWLN EKQDAKVGED NKDMIFYKKF DEFADDGFAP | |
| | FFYFDKFGNY LKRRSRNTTK EIKLHFGNDD LLEGWDMNKE PEYWSFILRD | |
| | RNQYYLGIGK KDGEIFHKKL GNSVEAVKEA YELENEADFY EKIDYKQLNI | |
| | DRFEGIAFPK KTKTEEAFRQ VCKKRADEFL GGDTYEFKIL LAIKKEYDDF | |
| | KARRQKEKDW DSKFSKEKMS KLIEYYITCL GKRDDWKRFN LNFRQPKEYE | |
| | DRSDFVRHIQ RQAYWIDPRK VSKDYVDKKV AEGEMFLFKV HNKDFYDFER | |
| | KSEDKKNHTA NLFTQYLLEL FSCENIKNIK SKDLIESIFE LDGKAEIRFR | |
| | PKTDDVKLKI YQKKGKDVTY ADKRDGNKEK EVIQHRRFAK DALTLHLKIR | |
| | LNFGKHVNLF DFNKLVNTEL FAKVPVKILG MDRGENNLIY YCFLDEHGEI | |
| | ENGKCGSLNR VGEQIITLED DKKVKEPVDY FQLLVDREGQ RDWEQKNWQK | |
| | MTRIKDLKKA YLGNVVSWIS KEMLSGIKEG VVTIGVLEDL NSNFKRTRFF | |
| | RERQVYQGFE KALVNKLGYL VDKKYDNYRN VYQFAPIVDS VEEMEKNKQI | |
| | GTLVYVPASY TSKICPHPKC GWRERLYMKN SASKEKIVGL LKSDGIKISY | |
| | DQKNDRFYFE YQWEQEHKSD GKKKKYSGVD KVFSNVSRMR WDVEQKKSID | |
| | FVDGTDGSIT NKLKSLLKGK GIELDNINQQ IVNQQKELGV EFFQSIIFYF | |
| | NLIMQIRNYD KEKSGSEADY IQCPSCLFDS RKPEMNGKLS AITNGDANGA | |
| | YNIARKGFMQ LCRIRENPQE PMKLITNREW DEAVREWDIY SAAQKIPVLS | |
| | EEN | |
| KDN25524_ | MLFQDFTHLY PLSKTVRFEL KPIDRTLEHI HAKNFLSQDE TMADMHQKVK | (SEQ |
| (modified) | VILDDYHRDF IADMMGEVKL TKLAEFYDVY LKFRKNPKDD ELQKQLKDLQ | ID NO: |
| hypothetical | AVLRKEIVKP IGNGGKYKAG YDRLFGAKLF KDGKELGDLA KFVIAQEGES | 118) |
| protein | SPKLAHLAHF EKFSTYFTGF HDNRKNMYSD EDKHTAIAYR LIHENLPRFI | |
| MBO_03467 | DNLQILTTIK QKHSALYDQI INELTASGLD VSLASHLDGY HKLLTQEGIT | |
| [Moraxella | AYNTLLGGIS GEAGSPKIQG INELINSHHN QHCHKSERIA KLRPLHKQIL | |
| bovoculi 237] | SDGMSVSFLP SKFADDSEMC QAVNEFYRHY ADVFAKVQSL FDGFDDHQKD | |
| >WP_052585281.1 | GIYVEHKNLN ELSKQAFGDF ALLGRVLDGY YVDVVNPEFN ERFAKAKTDN | |
| type V CRISPR- | AKAKLTKEKD KFIKGVHSLA SLEQAIEHYT ARHDDESVQA GKLGQYFKHG | |
| associated | LAGVDNPIQK IHNNHSTIKG FLERERPAGE RALPKIKSGK NPEMTQLRQL | |
| protein Cpf1 | KELLDNALNV AHFAKLLTTK TTLDNQDGNF YGEFGVLYDE LAKIPTLYNK | |
| [Moraxella | VRDYLSQKPF STEKYKLNFG NPTLLNGWDL NKEKDNFGVI LQKDGCYYLA | |
| bovoculi] | LLDKAHKKVF DNAPNTGKSI YQKMIYKYLE VRKQFPKVFF SKEAIAINYH | |
| | PSKELVEIKD KGRQRSDDER LKLYRFILEC LKIHPKYDKK FEGAIGDIQL | |
| | FKKDKKGREV PISEKDLFDK INGIFSSKPK LEMEDFFIGE FKRYNPSQDL | |
| | VDQYNIYKKI DSNDNRKKEN FYNNHPKFKK DLVRYYYESM CKHEEWEESF | |
| | EFSKKLQDIG CYVDVNELFT EIETRRLNYK ISFCNINADY IDELVEQGQL | |
| | YLFQIYNKDF SPKAHGKPNL HTLYFKALFS EDNLADPIYK LNGEAQIFYR | |
| | KASLDMNETT IHRAGEVLEN KNPDNPKKRQ FVYDIIKDKR YTQDKFMLHV | |
| | PITMNFGVQG MTIKEFNKKV NQSIQQYDEV NVIGIDRGER HLLYLTVINS | |
| | KGEILEQCSL NDITTASANG TQMTTPYHKI LDKREIERLN ARVGWGEIET | |
| | IKELKSGYLS HVVHQISQLM LKYNAIVVLE DLNFGFKRGR FKVEKQIYQN | |
| | FENALIKKLN HLVLKDKADD EIGSYKNALQ LTNNFTDLKS IGKQTGFLFY | |
| | VPAWNTSKID PETGFVDLLK PRYENIAQSQ AFFGKFDKIC YNADKDYFEF | |
| | HIDYAKFTDK AKNSRQIWTI CSHGDKRYVY DKTANQNKGA AKGINVNDEL | |
| | KSLFARHHIN EKQPNLVMDI CQNNDKEFHK SLMYLLKTLL ALRYSNASSD | |
| | EDFILSPVAN DEGVFFNSAL ADDTQPQNAD ANGAYHIALK GLWLLNELKN | |
| | SDDLNKVKLA IDNQTWLNFA QNR | |
| KKT48220_ | MENIFDQFIG KYSLSKTLRF ELKPVGKTED FLKINKVFEK DQTIDDSYNQ | (SEQ |
| (modified) | AKFYFDSLHQ KFIDAALASD KTSELSFQNF ADVLEKQNKI ILDKKREMGA | ID NO: |
| hypothetical | LRKRDKNAVG IDRLQKEIND AEDIIQKEKE KIYKDVRTLF DNEAESWKTY | 119) |
| protein | YQEREVDGKK ITFSKADLKQ KGADFLTAAG ILKVLKYEFP EEKEKEFQAK | |
| UW39_C0001G0 | NQPSLFVEEK ENPGQKRYIF DSFDKFAGYL TKFQQTKKNL YAADGTSTAV | |
| 044 | ATRIADNFII FHQNTKVFRD KYKNNHTDLG FDEENIFEIE RYKNCLLQRE | |
| [Parcubacteria | IEHIKNENSY NKIIGRINKK IKEYRDQKAK DTKLTKSDFP FFKNLDKQIL | |
| bacterium | GEVEKEKQLI EKTREKTEED VLIERFKEFI ENNEERFTAA KKLMNAFCNG | |
| GW2011_GWC2 | EFESEYEGIY LKNKAINTIS RRWFVSDRDF ELKLPQQKSK NKSEKNEPKV | |
| _44_17] | KKFISIAEIK NAVEELDGDI FKAVFYDKKI IAQGGSKLEQ FLVIWKYEFE | |
| | YLFRDIEREN GEKLLGYDSC LKIAKQLGIF PQEKEAREKA TAVIKNYADA | |
| | GLGIFQMMKY FSLDDKDRKN TPGQLSTNFY AEYDGYYKDF EFIKYYNEFR | |
| | NFITKKPFDE DKIKLNFENG ALLKGWDENK EYDFMGVILK KEGRLYLGIM | |
| | HKNHRKLFQS MGNAKGDNAN RYQKMIYKQI ADASKDVPRL LLTSKKAMEK | |
| | FKPSQEILRI KKEKTFKRES KNFSLRDLHA LIEYYRNCIP QYSNWSFYDF | |
| | QFQDTGKYQN IKEFTDDVQK YGYKISFRDI DDEYINQALN EGKMYLFEWV | |
| | NKDIYNTKNG SKNLHTLYFE HILSAENLND PVFKLSGMAE IFQRQPSVNE | |
| | REKITTQKNQ CILDKGDRAY KYRRYTEKKI MFHMSLVLNT GKGEIKQVQF | |
| | NKIINQRISS SDNEMRVNVI GIDRGEKNLL YYSVVKQNGE IIEQASLNEI | |
| | NGVNYRDKLI EREKERLKNR QSWKPVVKIK DLKKGYISHV IHKICQLIEK | |
| | YSAIVVLEDL NMRFKQIRGG IERSVYQQFE KALIDKLGYL VFKDNRDLRA | |
| | PGGVLNGYQL SAPFVSFEKM RKQTGILFYT QAEYTSKTDP ITGFRKNVYI | |
| | SNSASLDKIK EAVKKFDAIG WDGKEQSYFF KYNPYNLADE KYKNSTVSKE | |
| | WAIFASAPRI RRQKGEDGYW KYDRVKVNEE FEKLLKVWNF VNPKATDIKQ | |
| | EIIKKEKAGD LQGEKELDGR LRNFWHSFIY LENLVLELRN SFSLQIKIKA | |
| | GEVIAVDEGV DFIASPVKPF FTTPNPYIPS NLCWLAVENA DANGAYNIAR | |
| | KGVMILKKIR EHAKKDPEFK KLPNLFISNA EWDEAARDWG KYAGTTALNL | |
| | DH | |
| WP_031492824 | MSSLTKFTNK YSKQLTIKNE LIPVGKTLEN IKENGLIDGD EQLNENYQKA | (SEQ |
| (modified) | KIIVDDFLRD FINKALNNTQ IGNWRELADA LNKEDEDNIE KLQDKIRGII | ID NO: |
| hypothetical | VSKFETFDLF SSYSIKKDEK IIDDDNDVEE EELDLGKKTS SFKYIFKKNL | 120) |
| protein | FKLVLPSYLK TTNQDKLKII SSFDNFSTYF RGFFENRKNI FTKKPISTSI | |
| [Succinivibrio | AYRIVHDNFP KFLDNIRCFN VWQTECPQLI VKADNYLKSK NVIAKDKSLA | |
| dextrinosolvens] | NYFTVGAYDY FLSQNGIDFY NNIIGGLPAF AGHEKIQGLN EFINQECQKD | |
| | SELKSKLKNR HAFKMAVLFK QILSDREKSF VIDEFESDAQ VIDAVKNFYA | |
| | EQCKDNNVIF NLLNLIKNIA FLSDDELDGI FIEGKYLSSV SQKLYSDWSK | |
| | LRNDIEDSAN SKQGNKELAK KIKTNKGDVE KAISKYEFSL SELNSIVHDN | |
| | TKFSDLLSCT LHKVASEKLV KVNEGDWPKH LKNNEEKQKI KEPLDALLEI | |
| | YNTLLIFNCK SFNKNGNFYV DYDRCINELS SWVYLYNKTR NYCTKKPYNT | |
| | DKFKLNFNSP QLGEGFSKSK ENDCLTLLFK KDDNYYVGII RKGAKINFDD | |
| | TQAIADNTDN CIFKMNYFLL KDAKKFIPKC SIQLKEVKAH FKKSEDDYIL | |
| | SDKEKFASPL VIKKSTFLLA TAHVKGKKGN IKKFQKEYSK ENPTEYRNSL | |
| | NEWIAFCKEF LKTYKAATIF DITTLKKAEE YADIVEFYKD VDNLCYKLEF | |
| | CPIKTSFIEN LIDNGDLYLF RINNKDFSSK STGTKNLHTL YLQAIFDERN | |
| | LNNPTIMLNG GAELFYRKES IEQKNRITHK AGSILVNKVC KDGTSLDDKI | |
| | RNEIYQYENK FIDTLSDEAK KVLPNVIKKE ATHDITKDKR FTSDKFFFHC | |
| | PLTINYKEGD TKQFNNEVLS FLRGNPDINI IGIDRGERNL IYVTVINQKG | |
| | EILDSVSFNT VTNKSSKIEQ TVDYEEKLAV REKERIEAKR SWDSISKIAT | |
| | LKEGYLSAIV HEICLLMIKH NAIVVLENLN AGFKRIRGGL SEKSVYQKFE | |
| | KMLINKLNYF VSKKESDWNK PSGLLNGLQL SDQFESFEKL GIQSGFIFYV | |
| | PAAYTSKIDP TTGFANVLNL SKVRNVDAIK SFFSNFNEIS YSKKEALFKF | |
| | SFDLDSLSKK GFSSFVKFSK SKWNVYTFGE RIIKPKNKQG YREDKRINLT | |
| | FEMKKLLNEY KVSFDLENNL IPNLTSANLK DTFWKELFFI FKTTLQLRNS | |
| | VTNGKEDVLI SPVKNAKGEF FVSGTHNKTL PQDCDANGAY HIALKGLMIL | |
| | ERNNLVREEK DTKKIMAISN VDWFEYVQKR RGVL | |
| KKT50231_ | MKPVGKTEDF LKINKVFEKD QTIDDSYNQA KFYFDSLHQK FIDAALASDK | (SEQ |
| (modified) | TSELSFQNFA DVLEKQNKII LDKKREMGAL RKRDKNAVGI DRLQKEINDA | ID NO: |
| hypothetical | EDIIQKEKEK IYKDVRTLFD NEAESWKTYY QEREVDGKKI TFSKADLKQK | 121) |
| protein | GADFLTAAGI LKVLKYEFPE EKEKEFQAKN QPSLFVEEKE NPGQKRYIFD | |
| UW40_ | SFDKFAGYLT KFQQTKKNLY AADGTSTAVA TRIADNFIIF HQNTKVFRDK | |
| C0007G0006 | YKNNHTDLGF DEENIFEIER YKNCLLQREI EHIKNENSYN KIIGRINKKI | |
| [Parcubacteria | KEYRDQKAKD TKLTKSDFPF FKNLDKQILG EVEKEKQLIE KTREKTEEDV | |
| bacterium | LIERFKEFIE NNEERFTAAK KLMNAFCNGE FESEYEGIYL KNKAINTISR | |
| GW2011_GWF2 | RWFVSDRDFE LKLPQQKSKN KSEKNEPKVK KFISIAEIKN AVEELDGDIF | |
| _44_17] | KAVFYDKKII AQGGSKLEQF LVIWKYEFEY LFRDIERENG EKLLGYDSCL | |
| | KIAKQLGIFP QEKEAREKAT AVIKNYADAG LGIFQMMKYF SLDDKDRKNT | |
| | PGQLSTNFYA EYDGYYKDFE FIKYYNEFRN FITKKPFDED KIKLNFENGA | |
| | LLKGWDENKE YDFMGVILKK EGRLYLGIMH KNHRKLFQSM GNAKGDNANR | |
| | YQKMIYKQIA DASKDVPRLL LTSKKAMEKF KPSQEILRIK KEKTFKRESK | |
| | NFSLRDLHAL IEYYRNCIPQ YSNWSFYDFQ FQDTGKYQNI KEFTDDVQKY | |
| | GYKISFRDID DEYINQALNE GKMYLFEWVN KDIYNTKNGS KNLHTLYFEH | |
| | ILSAENLNDP VFKLSGMAEI FQRQPSVNER EKITTQKNQC ILDKGDRAYK | |
| | YRRYTEKKIM FHMSLVLNTG KGEIKQVQFN KIINQRISSS DNEMRVNVIG | |
| | IDRGEKNLLY YSVVKQNGEI IEQASLNEIN GVNYRDKLIE REKERLKNRQ | |
| | SWKPVVKIKD LKKGYISHVI HKICQLIEKY SAIVVLEDLN MRFKQIRGGI | |
| | ERSVYQQFEK ALIDKLGYLV FKDNRDLRAP GGVLNGYQLS APFVSFEKMR | |
| | KQTGILFYTQ AEYTSKTDPI TGFRKNVYIS NSASLDKIKE AVKKFDAIGW | |
| | DGKEQSYFFK YNPYNLADEK YKNSTVSKEW AIFASAPRIR RQKGEDGYWK | |
| | YDRVKVNEEF EKLLKVWNFV NPKATDIKQE IIKKEKAGDL QGEKELDGRL | |
| | RNFWHSFIYL FNLVLELRNS FSLQIKIKAG EVIAVDEGVD FIASPVKPFF | |
| | TTPNPYIPSN LCWLAVENAD ANGAYNIARK GVMILKKIRE HAKKDPEFKK | |
| | LPNLFISNAE WDEAARDWGK YAGTTALNLD H | |
| WP_004356401 | MKVMENYQEF TNLFQLNKTL RFELKPIGKT CELLEEGKIF ASGSFLEKDK | (SEQ |
| (modified) | VRADNVSYVK KEIDKKHKIF IEETLSSFSI SNDLLKQYFD CYNELKAFKK | ID NO: |
| hypothetical | DCKSDEEEVK KTALRNKCTS IQRAMREAIS QAFLKSPQKK LLAIKNLIEN | 122) |
| protein | VFKADENVQH FSEFTSYFSG FETNRENFYS DEEKSTSIAY RLVHDNLPIF | |
| [Prevotella | IKNIYIFEKL KEQFDAKTLS EIFENYKLYV AGSSLDEVFS LEYFNNTLTQ | |
| disiens] | KGIDNYNAVI GKIVKEDKQE IQGLNEHINL YNQKHKDRRL PFFISLKKQI | |
| | LSDREALSWL PDMFKNDSEV IKALKGFYIE DGFENNVLTP LATLLSSLDK | |
| | YNLNGIFIRN NEALSSLSQN VYRNFSIDEA IDANAELQTF NNYELIANAL | |
| | RAKIKKETKQ GRKSFEKYEE YIDKKVKAID SLSIQEINEL VENYVSEFNS | |
| | NSGNMPRKVE DYFSLMRKGD FGSNDLIENI KTKLSAAEKL LGTKYQETAK | |
| | DIFKKDENSK LIKELLDATK QFQHFIKPLL GTGEEADRDL VFYGDFLPLY | |
| | EKFEELTLLY NKVRNRLTQK PYSKDKIRLC FNKPKLMTGW VDSKTEKSDN | |
| | GTQYGGYLFR KKNEIGEYDY FLGISSKAQL FRKNEAVIGD YERLDYYQPK | |
| | ANTIYGSAYE GENSYKEDKK RLNKVIIAYI EQIKQTNIKK SIIESISKYP | |
| | NISDDDKVTP SSLLEKIKKV SIDSYNGILS FKSFQSVNKE VIDNLLKTIS | |
| | PLKNKAEFLD LINKDYQIFT EVQAVIDEIC KQKTFIYFPI SNVELEKEMG | |
| | DKDKPLCLFQ ISNKDLSFAK TFSANLRKKR GAENLHTMLF KALMEGNQDN | |
| | LDLGSGAIFY RAKSLDGNKP THPANEAIKC RNVANKDKVS LFTYDIYKNR | |
| | RYMENKFLFH LSIVQNYKAA NDSAQLNSSA TEYIRKADDL HIIGIDRGER | |
| | NLLYYSVIDM KGNIVEQDSL NIIRNNDLET DYHDLLDKRE KERKANRQNW | |
| | EAVEGIKDLK KGYLSQAVHQ IAQLMLKYNA IIALEDLGQM FVTRGQKIEK | |
| | AVYQQFEKSL VDKLSYLVDK KRPYNELGGI LKAYQLASSI TKNNSDKQNG | |
| | FLFYVPAWNT SKIDPVTGFT DLLRPKAMTI KEAQDFFGAF DNISYNDKGY | |
| | FEFETNYDKF KIRMKSAQTR WTICTFGNRI KRKKDKNYWN YEEVELTEEF | |
| | KKLFKDSNID YENCNLKEEI QNKDNRKFFD DLIKLLQLTL QMRNSDDKGN | |
| | DYIISPVANA EGQFFDSRNG DKKLPLDADA NGAYNIARKG LWNIRQIKQT | |
| | KNDKKLNLSI SSTEWLDFVR EKPYLK | |
| CCB70584_(modified) | MTNKFTNQYS LSKTLRFELI PQGKTLEFIQ EKGLLSQDKQ RAESYQEMKK | (SEQ |
| Protein of | TIDKFHKYFI DLALSNAKLT HLETYLELYN KSAETKKEQK FKDDLKKVQD | ID NO: |
| unknown | NLRKEIVKSF SDGDAKSIFA ILDKKELITV ELEKWFENNE QKDIYFDEKF | 123) |
| function | KTFTTYFTGF HQNRKNMYSV EPNSTAIAYR LIHENLPKFL ENAKAFEKIK | |
| [Flavobacterium | QVESLQVNFR ELMGEFGDEG LIFVNELEEM FQINYYNDVL SQNGITIYNS | |
| branchiophilum | IISGFTKNDI KYKGLNEYIN NYNQTKDKKD RLPKLKQLYK QILSDRISLS | |
| FL-15] | FLPDAFTDGK QVLKAIFDFY KINLLSYTIE GQEESQNLLL LIRQTIENLS | |
| | SFDTQKIYLK NDTHLTTISQ QVFGDFSVFS TALNYWYETK VNPKFETEYS | |
| | KANEKKREIL DKAKAVFTKQ DYFSIAFLQE VLSEYILTLD HTSDIVKKHS | |
| | SNCIADYFKN HFVAKKENET DKTFDFIANI TAKYQCIQGI LENADQYEDE | |
| | LKQDQKLIDN LKFFLDAILE LLHFIKPLHL KSESITEKDT AFYDVFENYY | |
| | EALSLLTPLY NMVRNYVTQK PYSTEKIKLN FENAQLLNGW DANKEGDYLT | |
| | TILKKDGNYF LAIMDKKHNK AFQKFPEGKE NYEKMVYKLL PGVNKMLPKV | |
| | FFSNKNIAYF NPSKELLENY KKETHKKGDT FNLEHCHTLI DFFKDSLNKH | |
| | EDWKYFDFQF SETKSYQDLS GFYREVEHQG YKINFKNIDS EYIDGLVNEG | |
| | KLFLFQIYSK DFSPFSKGKP NMHTLYWKAL FEEQNLQNVI YKLNGQAEIF | |
| | FRKASIKPKN IILHKKKIKI AKKHFIDKKT KTSEIVPVQT IKNLNMYYQG | |
| | KISEKELTQD DLRYIDNFSI FNEKNKTIDI IKDKRFTVDK FQFHVPITMN | |
| | FKATGGSYIN QTVLEYLQNN PEVKIIGLDR GERHLVYLTL IDQQGNILKQ | |
| | ESLNTITDSK ISTPYHKLLD NKENERDLAR KNWGTVENIK ELKEGYISQV | |
| | VHKIATLMLE ENAIVVMEDL NFGFKRGRFK VEKQIYQKLE KMLIDKLNYL | |
| | VLKDKQPQEL GGLYNALQLT NKFESFQKMG KQSGFLFYVP AWNTSKIDPT | |
| | TGFVNYFYTK YENVDKAKAF FEKFEAIRFN AEKKYFEFEV KKYSDFNPKA | |
| | EGTQQAWTIC TYGERIETKR QKDQNNKFVS TPINLTEKIE DFLGKNQIVY | |
| | GDGNCIKSQI ASKDDKAFFE TLLYWFKMTL QMRNSETRTD IDYLISPVMN | |
| | DNGTFYNSRD YEKLENPTLP KDADANGAYH IAKKGLMLLN KIDQADLTKK | |
| | VDLSISNRDW LQFVQKNK | |
| WP_005398606 | MFEKLSNIVS ISKTIRFKLI PVGKTLENIE KLGKLEKDFE RSDFYPILKN | (SEQ |
| (modified) | ISDDYYRQYI KEKLSDLNLD WQKLYDAHEL LDSSKKESQK NLEMIQAQYR | ID NO: |
| hypothetical | KVLFNILSGE LDKSGEKNSK DLIKNNKALY GKLFKKQFIL EVLPDFVNNN | 124) |
| protein | DSYSEEDLEG LNLYSKFTTR LKNFWETRKN VFTDKDIVTA IPFRAVNENF | |
| [Helcococcus | GFYYDNIKIF NKNIEYLENK IPNLENELKE ADILDDNRSV KDYFTPNGFN | |
| kunzii] | YVITQDGIDV YQAIRGGFTK ENGEKVQGIN EILNLTQQQL RRKPETKNVK | |
| | LGVLTKLRKQ ILEYSESTSF LIDQIEDDND LVDRINKFNV SFFESTEVSP | |
| | SLFEQIERLY NALKSIKKEE VYIDARNTQK FSQMLFGQWD VIRRGYTVKI | |
| | TEGSKEEKKK YKEYLELDET SKAKRYLNIR EIEELVNLVE GFEEVDVFSV | |
| | LLEKFKMNNI ERSEFEAPIY GSPIKLEAIK EYLEKHLEEY HKWKLLLIGN | |
| | DDLDTDETFY PLLNEVISDY YIIPLYNLTR NYLTRKHSDK DKIKVNFDFP | |
| | TLADGWSESK ISDNRSIILR KGGYYYLGIL IDNKLLINKK NKSKKIYEIL | |
| | IYNQIPEFSK SIPNYPFTKK VKEHFKNNVS DFQLIDGYVS PLIITKEIYD | |
| | IKKEKKYKKD FYKDNNTNKN YLYTIYKWIE FCKQFLYKYK GPNKESYKEM | |
| | YDFSTLKDTS LYVNLNDFYA DVNSCAYRVL FNKIDENTID NAVEDGKLLL | |
| | FQIYNKDFSP ESKGKKNLHT LYWLSMFSEE NLRTRKLKLN GQAEIFYRKK | |
| | LEKKPIIHKE GSILLNKIDK EGNTIPENIY HECYRYLNKK IGREDLSDEA | |
| | IALFNKDVLK YKEARFDIIK DRRYSESQFF FHVPITFNWD IKTNKNVNQI | |
| | VQGMIKDGEI KHIIGIDRGE RHLLYYSVID LEGNIVEQGS LNTLEQNRFD | |
| | NSTVKVDYQN KLRTREEDRD RARKNWTNIN KIKELKDGYL SHVVHKLSRL | |
| | IIKYEAIVIM ENLNQGFKRG RFKVERQVYQ KFELALMNKL SALSFKEKYD | |
| | ERKNLEPSGI LNPIQACYPV DAYQELQGQN GIVFYLPAAY TSVIDPVTGF | |
| | TNLFRLKSIN SSKYEEFIKK FKNIYFDNEE EDFKFIFNYK DFAKANLVIL | |
| | NNIKSKDWKI STRGERISYN SKKKEYFYVQ PTEFLINKLK ELNIDYENID | |
| | IIPLIDNLEE KAKRKILKAL FDTFKYSVQL RNYDFENDYI ISPTADDNGN | |
| | YYNSNEIDID KTNLPNNGDA NGAFNIARKG LLLKDRIVNS NESKVDLKIK | |
| | NEDWINFIIS | |
| WP_021736722 | MTQFEGFTNL YQVSKTLRFE LIPQGKTLKH IQEQGFIEED KARNDHYKEL | (SEQ |
| (modified) | KPIIDRIYKT YADQCLQLVQ LDWENLSAAI DSYRKEKTEE TRNALIEEQA | ID NO: |
| CRISPR- | TYRNAIHDYF IGRTDNLTDA INKRHAEIYK GLFKAELFNG KVLKQLGTVT | 125 |
| associated | TTEHENALLR SFDKFTTYFS GFYENRKNVF SAEDISTAIP HRIVQDNFPK | |
| protein Cpf1, | FKENCHIFTR LITAVPSLRE HFENVKKAIG IFVSTSIEEV FSFPFYNQLL | |
| subtype | TQTQIDLYNQ LLGGISREAG TEKIKGLNEV LNLAIQKNDE TAHIIASLPH | |
| PREFRAN | RFIPLFKQIL SDRNTLSFIL EEFKSDEEVI QSFCKYKTLL RNENVLETAE | |
| [Acidaminococcus | ALFNELNSID LTHIFISHKK LETISSALCD HWDTLRNALY ERRISELTGK | |
| sp. BV3L6] | ITKSAKEKVQ RSLKHEDINL QEIISAAGKE LSEAFKQKTS EILSHAHAAL | |
| | DQPLPTTLKK QEEKEILKSQ LDSLLGLYHL LDWFAVDESN EVDPEFSARL | |
| | TGIKLEMEPS LSFYNKARNY ATKKPYSVEK FKLNFQMPTL ASGWDVNKEK | |
| | NNGAILFVKN GLYYLGIMPK QKGRYKALSF EPTEKTSEGF DKMYYDYFPD | |
| | AAKMIPKCST QLKAVTAHFQ THTTPILLSN NFIEPLEITK EIYDLNNPEK | |
| | EPKKFQTAYA KKTGDQKGYR EALCKWIDFT RDFLSKYTKT TSIDLSSLRP | |
| | SSQYKDLGEY YAELNPLLYH ISFQRIAEKE IMDAVETGKL YLFQIYNKDF | |
| | AKGHHGKPNL HTLYWTGLFS PENLAKTSIK LNGQAELFYR PKSRMKRMAH | |
| | RLGEKMLNKK LKDQKTPIPD TLYQELYDYV NHRLSHDLSD EARALLPNVI | |
| | TKEVSHEIIK DRRFTSDKFF FHVPITLNYQ AANSPSKFNQ RVNAYLKEHP | |
| | ETPIIGIDRG ERNLIYITVI DSTGKILEQR SLNTIQQFDY QKKLDNREKE | |
| | RVAARQAWSV VGTIKDLKQG YLSQVIHEIV DLMIHYQAVV VLENLNFGFK | |
| | SKRTGIAEKA VYQQFEKMLI DKLNCLVLKD YPAEKVGGVL NPYQLTDQFT | |
| | SFAKMGTQSG FLFYVPAPYT SKIDPLTGFV DPFVWKTIKN HESRKHFLEG | |
| | FDFLHYDVKT GDFILHFKMN RNLSFQRGLP GFMPAWDIVF EKNETQFDAK | |
| | GTPFIAGKRI VPVIENHRFT GRYRDLYPAN ELIALLEEKG IVFRDGSNIL | |
| | PKLLENDDSH AIDTMVALIR SVLQMRNSNA ATGEDYINSP VRDLNGVCFD | |
| | SRFQNPEWPM DADANGAYHI ALKGQLLLNH LKESKDLKLQ NGISNQDWLA | |
| | YIQELRN | |
| WP_004339290 | MSIYQEFVNK YSLSKTLRFE LIPQGKTLEN IKARGLILDD EKRAKDYKKA | (SEQ |
| (modified) | KQIIDKYHQF FIEEILSSVC ISEDLLQNYS DVYFKLKKSD DDNLQKDFKS | ID NO: |
| hypothetical | AKDTIKKQIS KYINDSEKFK NLFNQNLIDA KKGQESDLIL WLKQSKDNGI | 126) |
| protein | ELFKANSDIT DIDEALEIIK SFKGWTTYFK GFHENRKNVY SSNDIPTSII | |
| [Francisella | YRIVDDNLPK FLENKAKYES LKDKAPEAIN YEQIKKDLAE ELTFDIDYKT | |
| tularensis] | SEVNQRVFSL DEVFEIANFN NYLNQSGITK FNTIIGGKFV NGENTKRKGI | |
| | NEYINLYSQQ INDKTLKKYK MSVLFKQILS DTESKSFVID KLEDDSDWVT | |
| | TMQSFYEQIA AFKTVEEKSI KETLSLLFDD LKAQKLDLSK IYFKNDKSLT | |
| | DLSQQVFDDY SVIGTAVLEY ITQQVAPKNL DNPSKKEQDL IAKKTEKAKY | |
| | LSLETIKLAL EEFNKHRDID KQCRFEEILS NFAAIPMIFD EIAQNKDNLA | |
| | QISIKYQNQG KKDLLQASAE EDVKAIKDLL DQTNNLLHRL KIFHISQSED | |
| | KANILDKDEH FYLVFEECYF ELANIVPLYN KIRNYITQKP YSDEKFKLNF | |
| | ENSTLASGWD KNKESANTAI LFIKDDKYYL GIMDKKHNKI FSDKAIEENK | |
| | GEGYKKIVYK QIADASKDIQ NLMIIDGKTV CKKGRKDRNG VNRQLLSLKR | |
| | KHLPENIYRI KETKSYLKNE ARFSRKDLYD FIDYYKDRLD YYDFEFELKP | |
| | SNEYSDFNDF TNHIGSQGYK LTFENISQDY INSLVNEGKL YLFQIYSKDF | |
| | SAYSKGRPNL HTLYWKALFD ERNLQDVVYK LNGEAELFYR KQSIPKKITH | |
| | PAKETIANKN KDNPKKESVF EYDLIKDKRF TEDKFFFHCP ITINFKSSGA | |
| | NKFNDEINLL LKEKANDVHI LSIDRGERHL AYYTLVDGKG NIIKQDNFNI | |
| | IGNDRMKTNY HDKLAAIEKD RDSARKDWKK INNIKEMKEG YLSQVVHEIA | |
| | KLVIEYNAIV VFEDLNFGFK RGRFKVEKQV YQKLEKMLIE KLNYLVFKDN | |
| | EFDKTGGVLR AYQLTAPFET FKKMGKQTGI IYYVPAGFTS KICPVTGFVN | |
| | QLYPKYESVS KSQEFFSKFD KICYNLDKGY FEFSFDYKNF GDKAAKGKWT | |
| | IASFGSRLIN FRNSDKNHNW DTREVYPTKE LEKLLKDYSI EYGHGECIKA | |
| | AICGESDKKF FAKLTSVLNT ILQMRNSKTG TELDYLISPV ADVNGNFFDS | |
| | RQAPKNMPQD ADANGAYHIG LKGLMLLDRI KNNQEGKKLN LVIKNEEYFE | |
| | FVQNRNN | |
| WP_022501477 | MNKAADNYTG GNYDEFIALS KVQKTLRNEL KPTPFTAEHI KQRGIISEDE | (SEQ |
| type V CRISPR- | YRAQQSLELK KIADEYYRNY ITHKLNDINN LDFYNLFDAI EEKYKKNDKD | ID NO: |
| associated | NRDKLDLVEK SKRGEIAKML SADDNFKSMF EAKLITKLLP DYVERNYTGE | 127 |
| protein Cpf1 | DKEKALETLA LFKGFTTYFK GYFKTRKNMF SGEGGASSIC HRIVNVNASI | |
| [Eubacterium sp. | FYDNLKTFMR IQEKAGDEIA LIEEELTEKL DGWRLEHIFS RDYYNEVLAQ | |
| CAG: 76] | KGIDYYNQIC GDINKHMNLY CQQNKFKANI FKMMKIQKQI MGISEKAFEI | |
| | PPMYQNDEEV YASFNEFISR LEEVKLTDRL INILQNINIY NTAKIYINAR | |
| | YYTNVSSYVY GGWGVIDSAI ERYLYNTIAG KGQSKVKKIE NAKKDNKFMS | |
| | VKELDSIVAE YEPDYFNAPY IDDDDNAVKA FGGQGVLGYF NKMSELLADV | |
| | SLYTIDYNSD DSLIENKESA LRIKKQLDDI MSLYHWLQTF IIDEVVEKDN | |
| | AFYAELEDIC CELENVVTLY DRIRNYVTKK PYSTQKFKLN FASPTLAAGW | |
| | SRSKEFDNNA IILLRNNKYY IAIFNVNNKP DKQIIKGSEE QRLSTDYKKM | |
| | VYNLLPGPNK MLPKVFIKSD TGKRDYNPSS YILEGYEKNR HIKSSGNFDI | |
| | NYCHDLIDYY KACINKHPEW KNYGFKFKET NQYNDIGQFY KDVEKQGYSI | |
| | SWAYISEEDI NKLDEEGKIY LFEIYNKDLS AHSTGRDNLH TMYLKNIFSE | |
| | DNLKNICIEL NGEAELFYRK SSMKSNITHK KDTILVNKTY INETGVRVSL | |
| | SDEDYMKVYN YYNNNYVIDT ENDKNLIDII EKIGHRKSKI DIVKDKRYTE | |
| | DKYFLYLPIT INYGIEDENV NSKIIEYIAK QDNMNVIGID RGERNLIYIS | |
| | VIDNKGNIIE QKSFNLVNNY DYKNKLKNME KTRDNARKNW QEIGKIKDVK | |
| | SGYLSGVISK IARMVIDYNA IIVMEDLNKG FKRGRFKVER QVYQKFENML | |
| | ISKLNYLVFK ERKADENGGI LRGYQLTYIP KSIKNVGKQC GCIFYVPAAY | |
| | TSKIDPATGF INIFDFKKYS GSGINAKVKD KKEFLMSMNS IRYINECSEE | |
| | YEKIGHRELF AFSFDYNNFK TYNVSSPVNE WTAYTYGERI KKLYKDGRWL | |
| | RSEVLNLTEN LIKLMEQYNI EYKDGHDIRE DISHMDETRN ADFICSLFEE | |
| | LKYTVQLRNS KSEAEDENYD RLVSPILNSS NGFYDSSDYM ENENNTTHTM | |
| | PKDADANGAY CIALKGLYEI NKIKQNWSDD KKFKENELYI NVTEWLDYIQ | |
| | NRRFE | |
| WP_014550095 | MSIYQEFVNK YSLSKTLRFE LIPQGKTLEN IKARGLILDD EKRAKDYKKA | (SEQ |
| type V CRISPR- | KQIIDKYHQF FIEEILSSVC ISEDLLQNYS DVYFKLKKSD DDNLQKDFKS | ID NO: |
| associated | AKDTIKKQIS EYIKDSEKFK NLFNQNLIDA KKGQESDLIL WLKQSKDNGI | 128) |
| protein Cpf1 | ELFKANSDIT DIDEALEIIK SFKGWTTYFK GFHENRKNVY SSNDIPTSII | |
| [Francisella | YRIVDDNLPK FLENKAKYES LKDKAPEAIN YEQIKKDLAE ELTFDIDYKT | |
| tularensis] | SEVNQRVFSL DEVFEIANFN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLFDD LKAQKLDLSK IYFKNDKSLT | |
| | DLSQQVFDDY SVIGTAVLEY ITQQVAPKNL DNPSKKEQDL IAKKTEKAKY | |
| | LSLETIKLAL EEFNKHRDID KQCRFEEILA NFAAIPMIFD EIAQNKDNLA | |
| | QISIKYQNQG KKDLLQASAE DDVKAIKDLL DQTNNLLHRL KIFHISQSED | |
| | KANILDKDEH FYLVFEECYF ELANIVPLYN KIRNYITQKP YSDEKFKLNF | |
| | ENSTLANGWD KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GNPQKGYEKF EFNIEDCRKF IDFYKESISK HPEWKDFGFR FSDTQRYNSI | |
| | DEFYREVENQ GYKLTFENIS ESYIDSVVNQ GKLYLFQIYN KDFSAYSKGR | |
| | PNLHTLYWKA LFDERNLQDV VYKLNGEAEL FYRKKSIPKK ITHPAKEAIA | |
| | NKNKDNPKKE SFFEYDLIKD KRFTEDKFFF HCPITINFKS SGANKFNDEI | |
| | NLLLKEKAND VHILSIDRGE RHLAYYTLVD GKGNIIKQDT FNIIGNDRMK | |
| | TNYHDKLAAI EKDRDSARKD WKKINNIKEM KEGYLSQVVH EIAKLVIEHN | |
| | AIVVFEDLNF GFKRGRFKVE KQVYQKLEKM LIEKLNYLVF KDNEFDKTGG | |
| | VLRAYQLTAP FETFKKMGKQ TGIIYYVPAG FTSKICPVTG FVNQLYPKYE | |
| | SVSKSQEFFS KFDKICYNLD KGYFEFSFDY KNFGDKAAKG KWTIASFGSR | |
| | LINFRNSDKN HNWDTREVYP TKELEKLLKD YSIEYGHGEC IKAAICGESD | |
| | KKFFAKLTSI LNTILQMRNS KTGTELDYLI SPVADVNGNF FDSRQAPKNM | |
| | PQDADANGAY HIGLKGLMLL DRIKNNQEGK KLNLVIKNEE YFEFVQNRNN | |
| WP_003034647 | MSIYQEFVNK YSLSKTLRFE LIPQGKTLEN IKARGLILDD EKRAKDYKKA | (SEQ |
| type V CRISPR- | KQIIDKYHQF FIEEILSSVC ISEDLLQNYS DVYFKLKKSD DDNLQKDFKS | ID NO: |
| associated | AKDTIKKQIS EYIKDSEKFK NLFNQNLIDA KKGQESDLIL WLKQSKDNGI | 129) |
| protein Cpf1 | ELFKANSDIT DIDEALEIIK SFKGWTTYFK GFHENRKNVY SSDDIPTSII | |
| [Francisella | YRIVDDNLPK FLENKAKYES LKDKAPEAIN YEQIKKDLAE ELTFDIDYKT | |
| tularensis] | SEVNQRVFSL DEVFEIANFN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLFDD LKAQKLDLSK IYFKNDKSLT | |
| | DLSQQVFDDY SVIGTAVLEY ITQQVAPKNL DNPSKKEQDL IAKKTEKAKY | |
| | LSLETIKLAL EEFNKHRDID KQCRFEEILA NFAAIPMIFD EIAQNKDNLA | |
| | QISLKYQNQG KKDLLQASAE EDVKAIKDLL DQTNNLLHRL KIFHISQSED | |
| | KANILDKDEH FYLVFEECYF ELANIVPLYN KIRNYITQKP YSDEKFKLNF | |
| | ENSTLANGWD KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GNPQKGYEKF EFNIEDCRKF IDFYKESISK HPEWKDFGFR FSDTQRYNSI | |
| | DEFYREVENQ GYKLTFENIS ESYIDSVVNQ GKLYLFQIYN KDFSAYSKGR | |
| | PNLHTLYWKA LFDERNLQDV VYKLNGEAEL FYRKQSIPKK ITHPAKEAIA | |
| | NKNKDNPKKE SVFEYDLIKD KRFTEDKFFF HCPITINFKS SGANKFNDEI | |
| | NLLLKEKAND VHILSIDRGE RHLAYYTLVD GKGNIIKQDT FNIIGNDRMK | |
| | TNYHDKLAAI EKDRDSARKD WKKINNIKEM KEGYLSQVVH EIAKLVIEHN | |
| | AIVVFEDLNF GFKRGRFKVE KQVYQKLEKM LIEKLNYLVF KDNEFDKTGG | |
| | VLRAYQLTAP FETFKKMGKQ TGIIYYVPAG FTSKICPVTG FVNQLYPKYE | |
| | SVSKSQEFFS KFDKICYNLD KGYFEFSFDY KNFGDKAAKG KWTIASFGSR | |
| | LINFRNSDKN HNWDTREVYP TKELEKLLKD YSIEYGHGEC IKAAICGESD | |
| | KKFFAKLTSV LNTILQMRNS KTGTELDYLI SPVADVNGNF FDSRQAPKNM | |
| | PQDADANGAY HIGLKGLMLL DRIKNNQEGK KLNLVIKNEE YFEFVQNRNN | |
| WP_003040289.1 | MSIYQEFVNK YSLSKTLRFE LIPQGKTLEN IKARGLILDD EKRAKDYKKA | (SEQ |
| type V CRISPR- | KQIIDKYHQF FIEEILSSVC ISEDLLQNYS DVYFKLKKSD DDNLQKDFKS | ID NO: |
| associated | AKDTIKKQIS EYIKDSEKFK NLFNQNLIDA KKGQESDLIL WLKQSKDNGI | 130) |
| protein Cpf1 | ELFKANSDIT DIDEALEIIK SFKGWTTYFK GFHENRKNVY SSNDIPTSII | |
| [Francisella | YRIVDDNLPK FLENKAKYES LKDKAPEAIN YEQIKKDLAE ELTFDIDYKT | |
| tularensis subsp. | SEVNQRVFSL DEVFEIANFN NYLNQSGITK FNTIIGGKFV NGENTKRKGI | |
| novicida U112] | NEYINLYSQQ INDKTLKKYK MSVLFKQILS DTESKSFVID KLEDDSDVVT | |
| | TMQSFYEQIA AFKTVEEKSI KETLSLLFDD LKAQKLDLSK IYFKNDKSLT | |
| | DLSQQVFDDY SVIGTAVLEY ITQQIAPKNL DNPSKKEQEL IAKKTEKAKY | |
| | LSLETIKLAL EEFNKHRDID KQCRFEEILA NFAAIPMIFD EIAQNKDNLA | |
| | QISIKYQNQG KKDLLQASAE DDVKAIKDLL DQTNNLLHKL KIFHISQSED | |
| | KANILDKDEH FYLVFEECYF ELANIVPLYN KIRNYITQKP YSDEKFKLNF | |
| | ENSTLANGWD KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GSPQKGYEKF EFNIEDCRKF IDFYKQSISK HPEWKDFGFR FSDTQRYNSI | |
| | DEFYREVENQ GYKLTFENIS ESYIDSVVNQ GKLYLFQIYN KDFSAYSKGR | |
| | PNLHTLYWKA LFDERNLQDV VYKLNGEAEL FYRKQSIPKK ITHPAKEAIA | |
| | NKNKDNPKKE SVFEYDLIKD KRFTEDKFFF HCPITINFKS SGANKFNDEI | |
| | NLLLKEKAND VHILSIDRGE RHLAYYTLVD GKGNIIKQDT FNIIGNDRMK | |
| | TNYHDKLAAI EKDRDSARKD WKKINNIKEM KEGYLSQVVH EIAKLVIEYN | |
| | AIVVFEDLNF GFKRGRFKVE KQVYQKLEKM LIEKLNYLVF KDNEFDKTGG | |
| | VLRAYQLTAP FETFKKMGKQ TGIIYYVPAG FTSKICPVTG FVNQLYPKYE | |
| | SVSKSQEFFS KFDKICYNLD KGYFEFSFDY KNFGDKAAKG KWTIASFGSR | |
| | LINFRNSDKN HNWDTREVYP TKELEKLLKD YSIEYGHGEC IKAAICGESD | |
| | KKFFAKLTSV LNTILQMRNS KTGTELDYLI SPVADVNGNF FDSRQAPKNM | |
| | PQDADANGAY HIGLKGLMLL GRIKNNQEGK KLNLVIKNEE YFEFVQNRNN | |
| KKQ38174 | MKSFDSFTNL YSLSKTLKFE MRPVGNTQKM LDNAGVFEKD KLIQKKYGKT | (SEQ |
| hypothetical | KPYFDRLHRE FIEEALTGVE LIGLDENFRT LVDWQKDKKN NVAMKAYENS | ID NO: |
| protein | LQRLRTEIGK IFNLKAEDWV KNKYPILGLK NKNTDILFEE AVFGILKARY | 131) |
| US54_C0016G0 | GEEKDTFIEV EEIDKTGKSK INQISIFDSW KGFTGYFKKF FETRKNFYKN | |
| 015 [Candidatus | DGTSTAIATR IIDQNLKRFI DNLSIVESVR QKVDLAETEK SFSISLSQFF | |
| Roizmanbacteria | SIDFYNKCLL QDGIDYYNKI IGGETLKNGE KLIGLNELIN QYRQNNKDQK | |
| bacterium | IPFFKLLDKQ ILSEKILFLD EIKNDTELIE ALSQFAKTAE EKTKIVKKLF | |
| GW2011_GWA2 | ADFVENNSKY DLAQIYISQE AFNTISNKWT SETETFAKYL FEAMKSGKLA | |
| 37_7] | KYEKKDNSYK FPDFIALSQM KSALLSISLE GHFWKEKYYK ISKFQEKTNW | |
| | EQFLAIFLYE FNSLFSDKIN TKDGETKQVG YYLFAKDLHN LILSEQIDIP | |
| | KDSKVTIKDF ADSVLTIYQM AKYFAVEKKR AWLAEYELDS FYTQPDTGYL | |
| | QFYDNAYEDI VQVYNKLRNY LTKKPYSEEK WKLNFENSTL ANGWDKNKES | |
| | DNSAVILQKG GKYYLGLITK GHNKIFDDRF QEKFIVGIEG GKYEKIVYKF | |
| | FPDQAKMFPK VCFSAKGLEF FRPSEEILRI YNNAEFKKGE TYSIDSMQKL | |
| | IDFYKDCLTK YEGWACYTFR HLKPTEEYQN NIGEFFRDVA EDGYRIDFQG | |
| | ISDQYIHEKN EKGELHLFEI HNKDWNLDKA RDGKSKTTQK NLHTLYFESL | |
| | FSNDNVVQNF PIKLNGQAEI FYRPKTEKDK LESKKDKKGN KVIDHKRYSE | |
| | NKIFFHVPLT LNRTKNDSYR FNAQINNFLA NNKDINIIGV DRGEKHLVYY | |
| | SVITQASDIL ESGSLNELNG VNYAEKLGKK AENREQARRD WQDVQGIKDL | |
| | KKGYISQVVR KLADLAIKHN AIIILEDLNM RFKQVRGGIE KSIYQQLEKA | |
| | LIDKLSFLVD KGEKNPEQAG HLLKAYQLSA PFETFQKMGK QTGIIFYTQA | |
| | SYTSKSDPVT GWRPHLYLKY FSAKKAKDDI AKFTKIEFVN DRFELTYDIK | |
| | DFQQAKEYPN KTVWKVCSNV ERFRWDKNLN QNKGGYTHYT NITENIQELF | |
| | TKYGIDITKD LLTQISTIDE KQNTSFFRDF IFYFNLICQI RNTDDSEIAK | |
| | KNGKDDFILS PVEPFFDSRK DNGNKLPENG DDNGAYNIAR KGIVILNKIS | |
| | QYSEKNENCE KMKWGDLYVS NIDWDNFVTQ ANARH | |
| WP_022097749 | MNGNRSIVYR EFVGVTPVAK TLRNELRPVG HTQEHIIQNG LIQEDELRQE | (SEQ |
| type V CRISPR- | KSTELKNIMD DYYREYIDKS LSGLTDLDFT LLFELMNSVQ SSLSKDNKKA | ID NO: |
| associated | LEKEHNKMRE QICTHLQSDS DYKNMFNAKL FKEILPDFIK NYNQYDVKDK | 132) |
| protein Cpf1 | AGKLETLALF NGFSTYFTDF FEKRKNVFTK EAVSTSIAYR IVHENSLIFL | |
| [Eubacterium | ANMTSYKKIS EKALDEIEVI EKNNQDKMGD WELNQIFNPD FYNMVLIQSG | |
| eligens CAG:72] | IDFYNEICGV VNAHMNLYCQ QTKNNYNLFK MRKLHKQILA YTSTSFEVPK | |
| | MFEDDMSVYN AVNAFIDETE KGNIIGKLKD IVNKYDELDE KRIYISKDFY | |
| | ETLSCFMSGN WNLITGCVEN FYDENIHAKG KSKEEKVKKA VKEDKYKSIN | |
| | DVNDLVEKYI DEKERNEFKN SNAKQYIREI SNIITDTETA HLEYDEHISL | |
| | IESEEKADEI KKRLDMYMNM YHWVKAFIVD EVLDRDEMFY SDIDDIYNIL | |
| | ENIVPLYNRV RNYVTQKPYT SKKIKLNFQS PTLANGWSQS KEFDNNAIIL | |
| | IRDNKYYLAI FNAKNKPDKK IIQGNSDKKN DNDYKKMVYN LLPGANKMLP | |
| | KVFLSKKGIE TFKPSDYIIS GYNAHKHIKT SENFDISFCR DLIDYFKNSI | |
| | EKHAEWRKYE FKFSATDSYN DISEFYREVE MQGYRIDWTY ISEADINKLD | |
| | EEGKIYLFQI YNKDFAENST GKENLHTMYF KNIFSEENLK NIVIKINGQA | |
| | ELFYRKASVK NPVKHKKDSV LVNKTYKNQL DNGDVVRIPI PDDIYNEIYK | |
| | MYNGYIKESD LSEAAKEYLD KVEVRTAQKD IVKDYRYTVD KYFIHTPITI | |
| | NYKVTARNNV NDMAVKYIAQ NDDIHVIGID RGERNLIYIS VIDSHGNIVK | |
| | QKSYNILNNY DYKKKLVEKE KTREYARKNW KSIGNIKELK EGYISGVVHE | |
| | IAMLMVEYNA IIAMEDLNYG FKRGRFKVER QVYQKFESML INKLNYFASK | |
| | GKSVDEPGGL LKGYQLTYVP DNIKNLGKQC GVIFYVPAAF TSKIDPSTGF | |
| | ISAFNFKSIS TNASRKQFFM QFDEIRYCAE KDMFSFGFDY NNFDTYNITM | |
| | GKTQWTVYTN GERLQSEFNN ARRTGKTKSI NLTETIKLLL EDNEINYADG | |
| | HDVRIDMEKM YEDKNSEFFA QLLSLYKLTV QMRNSYTEAE EQEKGISYDK | |
| | IISPVINDEG EFFDSDNYKE SDDKECKMPK DADANGAYCI ALKGLYEVLK | |
| | IKSEWTEDGF DRNCLKLPHA EWLDFIQNKR YE | |
| WP_021739647 | MIKKTIDTVL NVRPIFVGIQ HLYFYEGPCR FGEGDELMPE YDAMMNQEMN | (SEQ |
| hypothetical | AAYVNEVVQH ETEGVHIMDP IYVERDDWFR SPEAMYEKMA EDIDKVDFYL | ID NO: |
| protein | FHFGIGRGDI YLEFAERYKK PVGAAPGLCC DGIGNTAAVK NRGLEAYAFM | 133) |
| [Eubacterium | SWDEFDTWMR VLRVRKCLKN TRVLLAVRWD SNRSYSSYDN FINQSDVTNK | |
| ramulus] | WGIQFRHVNV HELLDQTHPV DPTTNPSTPG RKALNINDED MKEIEKITDE | |
| | LIANAEACTM EPDMVKKTIQ AYYTVQKLLD AYDCNAFTAP CPDLCSTRRF | |
| | SEEKFTLCMT HSLNDENGIS SACEYDINSV IGKVIMTNLS GKAPYMGNTN | |
| | AIVFDKEGHM IPFHKFNDNT IEDIADKTNL YMTFHSTPNR NLKGLKAEKE | |
| | RYRLAPFAYS GFGATIRYDF AQDIGQVITM IRISPDATKI FIAKGTISGG | |
| | AGYEMKNCDQ GVFFNVADKV DFYHKQQYFG NHTVLAYGDY VEELKMLAEA | |
| | LGIEAVIA | |
| gi\|800943167 | MKNFSNLYQV SKTVRFELKP IGNTLENIKN KSLLKNDSIR AESYQKMKKT | (SEQ |
| WP_045971446.1 | IDEFHKYFID LALNNKKLSY LNEYIALYTQ SAEAKKEDKF KADFKKVQDN | ID NO: |
| type V CRISPR- | LRKEIVSSFT EGEAKAIFSV LDKKELITIE LEKWKNENNL AVYLDESFKS | 134) |
| associated | FTTYFTGFHQ NRKNMYSAEA NSTAIAYRLI HENLPKFIEN SKAFEKSSQI | |
| protein Cpf1 | AELQPKIEKL YKEFEAYLNV NSISELFEID YFNEVLTQKG ITVYNNIIGG | |
| [Flavobacterium | RTATEGKQKI QGLNEIINLY NQTKPKNERL PKLKQLYKQI LSDRISLSFL | |
| sp. 316] | PDAFTEGKQV LKAVFEFYKI NLLSYKQDGV EESQNLLELI QQVVKNLGNQ | |
| | DVNKIYLKND TSLTTIAQQL FGDFSVFSAA LQYRYETVVN PKYTAEYQKA | |
| | NEAKQEKLDK EKIKFVKQDY FSIAFLQEVV ADYVKTLDEN LDWKQKYTPS | |
| | CIADYFTTHF IAKKENEADK TFNFIANIKA KYQCIQGILE QADDYEDELK | |
| | QDQKLIDNIK FFLDAILEVV HFIKPLHLKS ESITEKDNAF YDVFENYYEA | |
| | LNVVTPLYNM VRNYVTQKPY STEKIKLNFE NAQLLNGWDA NKEKDYLTTI | |
| | LKRDGNYFLA IMDKKHNKTF QQFTEDDENY EKIVYKLLPG VNKMLPKVFF | |
| | SNKNIAFFNP SKEILDNYKN NTHKKGATFN LKDCHALIDF FKDSLNKHED | |
| | WKYFDFQFSE TKTYQDLSGF YKEVEHQGYK INFKKVSVSQ IDTLIEEGKM | |
| | YLFQIYNKDF SPYAKGKPNM HTLYWKALFE TQNLENVIYK LNGQAEIFFR | |
| | KASIKKKNII THKAHQPIAA KNPLTPTAKN TFAYDLIKDK RYTVDKFQFH | |
| | VPITMNFKAT GNSYINQDVL AYLKDNPEVN IIGLDRGERH LVYLTLIDQK | |
| | GTILLQESLN VIQDEKTHTP YHTLLDNKEI ARDKARKNWG SIESIKELKE | |
| | GYISQVVHKI TKMMIEHNAI VVMEDLNFGF KRGRFKVEKQ IYQKLEKMLI | |
| | DKLNYLVLKD KQPHELGGLY NALQLTNKFE SFQKMGKQSG FLFYVPAWNT | |
| | SKIDPTTGFV NYFYTKYENV EKAKTFFSKF DSILYNKTKG YFEFVVKNYS | |
| | DFNPKAADTR QEWTICTHGE RIETKRQKEQ NNNFVSTTIQ LTEQFVNFFE | |
| | KVGLDLSKEL KTQLIAQNEK SFFEELFHLL KLTLQMRNSE SHTEIDYLIS | |
| | PVANEKGIFY DSRKATASLP IDADANGAYH IAKKGLWIME QINKTNSEDD | |
| | LKKVKLAISN REWLQYVQQV QKK | |
| WP_044110123. | MKQFTNLYQL SKTLRFELKP IGKTLEHINA NGFIDNDAHR AESYKKVKKL | (SEQ |
| 1 | IDDYHKDYIE NVLNNFKLNG EYLQAYFDLY SQDTKDKQFK DIQDKLRKSI | ID NO: |
| type V CRISPR- | ASALKGDDRY KTIDKKELIR QDMKTFLKKD TDKALLDEFY EFTTYFTGYH | 135) |
| associated | ENRKNMYSDE AKSTAIAYRL IHDNLPKFID NIAVFKKIAN TSVADNFSTI | |
| protein Cpf1 | YKNFEEYLNV NSIDEIFSLD YYNIVLTQTQ IEVYNSIIGG RTLEDDTKIQ | |
| [Prevotella | GINEFVNLYN QQLANKKDRL PKLKPLFKQI LSDRVQLSWL QEEFNTGADV | |
| brevis] | LNAVKEYCTS YFDNVEESVK VLLTGISDYD LSKIYITNDL ALTDVSQRMF | |
| | GEWSIIPNAI EQRLRSDNPK KTNEKEEKYS DRISKLKKLP KSYSLGYINE | |
| | CISELNGIDI ADYYATLGAI NTESKQEPSI PTSIQVHYNA LKPILDTDYP | |
| | REKNLSQDKL TVMQLKDLLD DFKALQHFIK PLLGNGDEAE KDEKFYGELM | |
| | QLWEVIDSIT PLYNKVRNYC TRKPFSTEKI KVNFENAQLL DGWDENKEST | |
| | NASIILRKNG MYYLGIMKKE YRNILTKPMP SDGDCYDKVV YKFFKDITTM | |
| | VPKCTTQMKS VKEHFSNSND DYTLFEKDKF IAPVVITKEI FDLNNVLYNG | |
| | VKKFQIGYLN NTGDSFGYNH AVEIWKSFCL KFLKAYKSTS IYDFSSIEKN | |
| | IGCYNDLNSF YGAVNLLLYN LTYRKVSVDY IHQLVDEDKM YLFMIYNKDF | |
| | STYSKGTPNM HTLYWKMLFD ESNLNDVVYK LNGQAEVFYR KKSITYQHPT | |
| | HPANKPIDNK NVNNPKKQSN FEYDLIKDKR YTVDKFMFHV PITLNFKGMG | |
| | NGDINMQVRE YIKTTDDLHF IGIDRGERHL LYICVINGKG EIVEQYSLNE | |
| | IVNNYKGTEY KTDYHTLLSE RDKKRKEERS SWQTIEGIKE LKSGYLSQVI | |
| | HKITQLMIKY NAIVLLEDLN MGFKRGRQKV ESSVYQQFEK ALIDKLNYLV | |
| | DKNKDANEIG GLLHAYQLTN DPKLPNKNSK QSGFLFYVPA WNTSKIDPVT | |
| | GFVNLLDTRY ENVAKAQAFF KKFDSIRYNK EYDRFEFKFD YSNFTAKAED | |
| | TRTQWTLCTY GTRIETFRNA EKNSNWDSRE IDLTTEWKTL FTQHNIPLNA | |
| | NLKEAILLQA NKNFYTDILH LMKLTLQMRN SVTGTDIDYM VSPVANECGE | |
| | FFDSRKVKEG LPVNADANGA YNIARKGLWL AQQIKNANDL SDVKLAITNK | |
| | EWLQFAQKKQ YLKD | |
| WP_036388671. | MLFQDFTHLY PLSKTMRFEL KPIGKTLEHI HAKNFLSQDE TMADMYQKVK | (SEQ |
| 1 | AILDDYHRDF IADMMGEVKL TKLAEFYDVY LKFRKNPKDD GLQKQLKDLQ | ID NO: |
| type V CRISPR- | AVLRKEIVKP IGNGGKYKAG YDRLFGAKLF KDGKELGDLA KFVIAQEGES | 136) |
| associated | SPKLAHLAHF EKFSTYFTGF HDNRKNMYSD EDKHTAITYR LIHENLPRFI | |
| protein Cpf1 | DNLQILATIK QKHSALYDQI INELTASGLD VSLASHLDGY HKLLTQEGIT | |
| [Moraxella | AYNTLLGGIS GEAGSRKIQG INELINSHHN QHCHKSERIA KLRPLHKQIL | |
| caprae] | SDGMGVSFLP SKFADDSEMC QAVNEFYRHY ADVFAKVQSL FDGFDDHQKD | |
| | GIYVEHKNLN ELSKQAFGDF ALLGRVLDGY YVDVVNPEFN ERFAKAKTDN | |
| | AKAKLTKEKD KFIKGVHSLA SLEQAIEHYT ARHDDESVQA GKLGQYFKHG | |
| | LAGVDNPIQK IHNNHSTIKG FLERERPAGE RALPKIKSGK NPEMTQLRQL | |
| | KELLDNALNV AHFAKLLTTK TTLDNQDGNF YGEFGALYDE LAKIPTLYNK | |
| | VRDYLSQKPF STEKYKLNFG NPTLLNGWDL NKEKDNFGII LQKDGCYYLA | |
| | LLDKAHKKVF DNAPNTGKNV YQKMIYKLLP GPNKMLPKVF FAKSNLDYYN | |
| | PSAELLDKYA QGTHKKGNNF NLKDCHALID FFKAGINKHP EWQHFGFKFS | |
| | PTSSYQDLSD FYREVEPQGY QVKFVDINAD YINELVEQGQ LYLFQIYNKD | |
| | FSPKAHGKPN LHTLYFKALF SKDNLANPIY KLNGEAQIFY RKASLDMNET | |
| | TIHRAGEVLE NKNPDNPKKR QFVYDIIKDK RYTQDKFMLH VPITMNFGVQ | |
| | GMTIKEFNKK VNQSIQQYDE VNVIGIDRGE RHLLYLTVIN SKGEILEQRS | |
| | LNDITTASAN GTQMTTPYHK ILDKREIERL NARVGWGEIE TIKELKSGYL | |
| | SHVVHQISQL MLKYNAIVVL EDLNFGFKRG RFKVEKQIYQ NFENALIKKL | |
| | NHLVLKDEAD DEIGSYKNAL QLTNNFTDLK SIGKQTGFLF YVPAWNTSKI | |
| | DPETGFVDLL KPRYENIAQS QAFFGKFDKI CYNADKDYFE FHIDYAKFTD | |
| | KAKNSRQIWK ICSHGDKRYV YDKTANQNKG ATKGINVNDE LKSLFARHHI | |
| | NDKQPNLVMD ICQNNDKEFH KSLIYLLKTL LALRYSNASS DEDFILSPVA | |
| | NDEGMFFNSA LADDTQPQNA DANGAYHIAL KGLWVLEQIK NSDDLNKVKL | |
| | AIDNQTWLNF AQNR | |
| WP_020988726. | MEDYSGFVNI YSIQKTLRFE LKPVGKTLEH IEKKGFLKKD KIRAEDYKAV | (SEQ |
| 1 | KKIIDKYHRA YIEEVFDSVL HQKKKKDKTR FSTQFIKEIK EFSELYYKTE | ID NO: |
| type V CRISPR- | KNIPDKERLE ALSEKLRKML VGAFKGEFSE EVAEKYKNLF SKELIRNEIE | 137) |
| associated | KFCETDEERK QVSNFKSFTT YFTGFHSNRQ NIYSDEKKST AIGYRIIHQN | |
| protein Cpf1 | LPKFLDNLKI IESIQRRFKD FPWSDLKKNL KKIDKNIKLT EYFSIDGFVN | |
| [Leptospira | VLNQKGIDAY NTILGGKSEE SGEKIQGLNE YINLYRQKNN IDRKNLPNVK | |
| inadai] | ILFKQILGDR ETKSFIPEAF PDDQSVLNSI TEFAKYLKLD KKKKSIIAEL | |
| | KKFLSSFNRY ELDGIYLAND NSLASISTFL FDDWSFIKKS VSFKYDESVG | |
| | DPKKKIKSPL KYEKEKEKWL KQKYYTISFL NDAIESYSKS QDEKRVKIRL | |
| | EAYFAEFKSK DDAKKQFDLL ERIEEAYAIV EPLLGAEYPR DRNLKADKKE | |
| | VGKIKDFLDS IKSLQFFLKP LLSAEIFDEK DLGFYNQLEG YYEEIDSIGH | |
| | LYNKVRNYLT GKIYSKEKFK LNFENSTLLK GWDENREVAN LCVIFREDQK | |
| | YYLGVMDKEN NTILSDIPKV KPNELFYEKM VYKLIPTPHM QLPRIIFSSD | |
| | NLSIYNPSKS ILKIREAKSF KEGKNFKLKD CHKFIDFYKE SISKNEDWSR | |
| | FDFKFSKTSS YENISEFYRE VERQGYNLDF KKVSKFYIDS LVEDGKLYLF | |
| | QIYNKDFSIF SKGKPNLHTI YFRSLFSKEN LKDVCLKLNG EAEMFFRKKS | |
| | INYDEKKKRE GHHPELFEKL KYPILKDKRY SEDKFQFHLP ISLNFKSKER | |
| | LNFNLKVNEF LKRNKDINII GIDRGERNLL YLVMINQKGE ILKQTLLDSM | |
| | QSGKGRPEIN YKEKLQEKEI ERDKARKSWG TVENIKELKE GYLSIVIHQI | |
| | SKLMVENNAI VVLEDLNIGF KRGRQKVERQ VYQKFEKMLI DKLNFLVFKE | |
| | NKPTEPGGVL KAYQLTDEFQ SFEKLSKQTG FLFYVPSWNT SKIDPRTGFI | |
| | DFLHPAYENI EKAKQWINKF DSIRFNSKMD WFEFTADTRK FSENLMLGKN | |
| | RVWVICTTNV ERYFTSKTAN SSIQYNSIQI TEKLKELFVD IPFSNGQDLK | |
| | PEILRKNDAV FFKSLLFYIK TTLSLRQNNG KKGEEEKDFI LSPVVDSKGR | |
| | FFNSLEASDD EPKDADANGA YHIALKGLMN LLVLNETKEE NLSRPKWKIK | |
| | NKDWLEFVWE RNR | |
| WP_023936172. | MPWIDLKDFT NLYPVSKTLR FELKPVGKTL ENIEKAGILK EDEHRAESYR | (SEQ |
| 1 | RVKKIIDTYH KVFIDSSLEN MAKMGIENEI KAMLQSFCEL YKKDHRTEGE | ID NO: |
| type V CRISPR- | DKALDKIRAV LRGLIVGAFT GVCGRRENTV QNEKYESLFK EKLIKEILPD | 138) |
| associated | FVLSTEAESL PFSVEEATRS LKEFDSFTSY FAGFYENRKN IYSTKPQSTA | |
| protein Cpf1 | IAYRLIHENL PKFIDNILVF QKIKEPIAKE LEHIRADFSA GGYIKKDERL | |
| [Porphyromonas | EDIFSLNYYI HVLSQAGIEK YNALIGKIVT EGDGEMKGLN EHINLYNQQR | |
| crevioricanis] | GREDRLPLFR PLYKQILSDR EQLSYLPESF EKDEELLRAL KEFYDHIAED | |
| | ILGRTQQLMT SISEYDLSRI YVRNDSQLTD ISKKMLGDWN AIYMARERAY | |
| | DHEQAPKRIT AKYERDRIKA LKGEESISLA NLNSCIAFLD NVRDCRVDTY | |
| | LSTLGQKEGP HGLSNLVENV FASYHEAEQL LSFPYPEENN LIQDKDNVVL | |
| | IKNLLDNISD LQRFLKPLWG MGDEPDKDER FYGEYNYIRG ALDQVIPLYN | |
| | KVRNYLTRKP YSTRKVKLNF GNSQLLSGWD RNKEKDNSCV ILRKGQNFYL | |
| | AIMNNRHKRS FENKVLPEYK EGEPYFEKMD YKFLPDPNKM LPKVFLSKKG | |
| | IEIYEPSPKL LEQYGHGTHK KGDTFSMDDL HELIDFFKHS IEAHEDWKQF | |
| | GFKFSDTATY ENVSSFYREV EDQGYKLSFR KVSESYVYSL IDQGKLYLFQ | |
| | IYNKDFSPCS KGTPNLHTLY WRMLFDERNL ADVIYKLDGK AEIFFREKSL | |
| | KNDHPTHPAG KPIKKKSRQK KGEESLFEYD LVKDRRYTMD KFQFHVPITM | |
| | NFKCSAGSKV NDMVNAHIRE AKDMHVIGID RGERNLLYIC VIDSRGTILD | |
| | QISLNTINDI DYHDLLESRD KDRQQERRNW QTIEGIKELK QGYLSQAVHR | |
| | IAELMVAYKA VVALEDLNMG FKRGRQKVES SVYQQFEKQL IDKLNYLVDK | |
| | KKRPEDIGGL LRAYQFTAPF KSFKEMGKQN GFLFYIPAWN TSNIDPTTGF | |
| | VNLFHAQYEN VDKAKSFFQK FDSISYNPKK DWFEFAFDYK NFTKKAEGSR | |
| | SMWILCTHGS RIKNFRNSQK NGQWDSEEFA LTEAFKSLFV RYEIDYTADL | |
| | KTAIVDEKQK DFFVDLLKLF KLTVQMRNSW KEKDLDYLIS PVAGADGRFF | |
| | DTREGNKSLP KDADANGAYN IALKGLWALR QIRQTSEGGK LKLAISNKEW | |
| | LQFVQERSYE KD | |
| WP_009217842. | MRKFNEFVGL YPISKTLRFE LKPIGKTLEH IQRNKLLEHD AVRADDYVKV | (SEQ |
| 1 | KKIIDKYHKC LIDEALSGFT FDTEADGRSN NSLSEYYLYY NLKKRNEQEQ | ID NO: |
| type V CRISPR- | KTFKTIQNNL RKQIVNKLTQ SEKYKRIDKK ELITTDLPDF LTNESEKELV | 139) |
| associated | EKFKNFTTYF TEFHKNRKNM YSKEEKSTAI AFRLINENLP KFVDNIAAFE | |
| protein Cpf1 | KVVSSPLAEK INALYEDFKE YLNVEEISRV FRLDYYDELL TQKQIDLYNA | |
| [Bacteroidetes | IVGGRTEEDN KIQIKGLNQY INEYNQQQTD RSNRLPKLKP LYKQILSDRE | |
| oral taxon 274] | SVSWLPPKFD SDKNLLIKIK ECYDALSEKE KVFDKLESIL KSLSTYDLSK | |
| | IYISNDSQLS YISQKMFGRW DIISKAIRED CAKRNPQKSR ESLEKFAERI | |
| | DKKLKTIDSI SIGDVDECLA QLGETYVKRV EDYFVAMGES EIDDEQTDTT | |
| | SFKKNIEGAY ESVKELLNNA DNITDNNLMQ DKGNVEKIKT LLDAIKDLQR | |
| | FIKPLLGKGD EADKDGVFYG EFTSLWTKLD QVTPLYNMVR NYLTSKPYST | |
| | KKIKLNFENS TLMDGWDLNK EPDNTTVIFC KDGLYYLGIM GKKYNRVFVD | |
| | REDLPHDGEC YDKMEYKLLP GANKMLPKVF FSETGIQRFL PSEELLGKYE | |
| | RGTHKKGAGF DLGDCRALID FFKKSIERHD DWKKFDFKFS DTSTYQDISE | |
| | FYREVEQQGY KMSFRKVSVD YIKSLVEEGK LYLFQIYNKD FSAHSKGTPN | |
| | MHTLYWKMLF DEENLKDVVY KLNGEAEVFF RKSSITVQSP THPANSPIKN | |
| | KNKDNQKKES KFEYDLIKDR RYTVDKFLFH VPITMNFKSV GGSNINQLVK | |
| | RHIRSATDLH IIGIDRGERH LLYLTVIDSR GNIKEQFSLN EIVNEYNGNT | |
| | YRTDYHELLD TREGERTEAR RNWQTIQNIR ELKEGYLSQV IHKISELAIK | |
| | YNAVIVLEDL NFGFMRSRQK VEKQVYQKFE KMLIDKLNYL VDKKKPVAET | |
| | GGLLRAYQLT GEFESFKTLG KQSGILFYVP AWNTSKIDPV TGFVNLFDTH | |
| | YENIEKAKVF FDKFKSIRYN SDKDWFEFVV DDYTRESPKA EGTRRDWTIC | |
| | TQGKRIQICR NHQRNNEWEG QEIDLTKAFK EHFEAYGVDI SKDLREQINT | |
| | QNKKEFFEEL LRLLRLTLQM RNSMPSSDID YLISPVANDT GCFFDSRKQA | |
| | ELKENAVLPM NADANGAYNI ARKGLLAIRK MKQEENDSAK ISLAISNKEW | |
| | LKFAQTKPYL ED | |
| WP_036890108. | MDSLKDFTNL YPVSKTLRFE LKPVGKTLEN IEKAGILKED EHRAESYRRV | (SEQ |
| 1 | KKIIDTYHKV FIDSSLENMA KMGIENEIKA MLQSFCELYK KDHRTEGEDK | ID NO: |
| type V CRISPR- | ALDKIRAVLR GLIVGAFTGV CGRRENTVQN EKYESLFKEK LIKEILPDFV | 140) |
| associated | LSTEAESLPF SVEEATRSLK EFDSFTSYFA GFYENRKNIY STKPQSTAIA | |
| protein Cpf1 | YRLIHENLPK FIDNILVFQK IKEPIAKELE HIRADFSAGG YIKKDERLED | |
| [Porphyromonas | IFSLNYYIHV LSQAGIEKYN ALIGKIVTEG DGEMKGLNEH INLYNQQRGR | |
| crevioricanis] | EDRLPLFRPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKMLPEYKEG EPYFEKMDYK FLPDPNKMLP KVFLSKKGIE | |
| | IYKPSPKLLE QYGHGTHKKG DTFSMDDLHE LIDFFKHSIE AHEDWKQFGF | |
| | KFSDTATYEN VSSFYREVED QGYKLSFRKV SESYVYSLID QGKLYLFQIY | |
| | NKDFSPCSKG TPNLHTLYWR MLFDERNLAD VIYKLDGKAE IFFREKSLKN | |
| | DHPTHPAGKP IKKKSRQKKG EESLFEYDLV KDRRYTMDKF QFHVPITMNF | |
| | KCSAGSKVND MVNAHIREAK DMHVIGIDRG ERNLLYICVI DSRGTILDQI | |
| | SLNTINDIDY HDLLESRDKD RQQEHRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGF LFYIPAWNTS NIDPTTGFVN | |
| | LFHVQYENVD KAKSFFQKFD SISYNPKKDW FEFAFDYKNF TKKAEGSRSM | |
| | WILCTHGSRI KNFRNSQKNG QWDSEEFALT EAFKSLFVRY EIDYTADLKT | |
| | AIVDEKQKDF FVDLLKLFKL TVQMRNSWKE KDLDYLISPV AGADGRFFDT | |
| | REGNKSLPKD ADANGAYNIA LKGLWALRQI RQTSEGGKLK LAISNKEWLQ | |
| | FVQERSYEKD | |
| WP_036887416. | MDSLKDFTNL YPVSKTLRFE LKPVGKTLEN IEKAGILKED EHRAESYRRV | (SEQ |
| 1 | KKIIDTYHKV FIDSSLENMA KMGIENEIKA MLQSFCELYK KDHRTEGEDK | ID NO: |
| type V CRISPR- | ALDKIRAVLR GLIVGAFTGV CGRRENTVQN EKYESLFKEK LIKEILPDFV | 141) |
| associated | LSTEAESLPF SVEEATRSLK EFDSFTSYFA GFYENRKNIY STKPQSTAIA | |
| protein Cpf1 | YRLIHENLPK FIDNILVFQK IKEPIAKELE HIRADFSAGG YIKKDERLED | |
| [Porphyromonas | IFSLNYYIHV LSQAGIEKYN ALIGKIVTEG DGEMKGLNEH INLYNQQRGR | |
| crevioricanis] | EDRLPLFRPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKVLPEYKEG EPYFEKMDYK FLPDPNKMLP KVFLSKKGIE | |
| | IYKPSPKLLE QYGHGTHKKG DTFSMDDLHE LIDFFKHSIE AHEDWKQFGF | |
| | KFSDTATYEN VSSFYREVED QGYKLSFRKV SESYVYSLID QGKLYLFQIY | |
| | NKDFSPCSKG TPNLHTLYWR MLFDERNLAD VIYKLDGKAE IFFREKSLKN | |
| | DHPTHPAGKP IKKKSRQKKG EESLFEYDLV KDRHYTMDKF QFHVPITMNF | |
| | KCSAGSKVND MVNAHIREAK DMHVIGIDRG ERNLLYICVI DSRGTILDQI | |
| | SLNTINDIDY HDLLESRDKD RQQERRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGF LFYIPAWNTS NIDPTTGFVN | |
| | LFHAQYENVD KAKSFFQKFD SISYNPKKDW FEFAFDYKNF TKKAEGSRSM | |
| | WILCTHGSRI KNFRNSQKNG QWDSEEFALT EAFKSLFVRY EIDYTADLKT | |
| | AIVDEKQKDF FVDLLKLFKL TVQMRNSWKE KDLDYLISPV AGADGRFFDT | |
| | REGNKSLPKD ADANGAYNIA LKGLWALRQI RQTSEGGKLK LAISNKEWLQ | |
| | FVQERSYEKD | |
| WP_023941260. | MDSLKDFTNL YPVSKTLRFE LKPVGKTLEN IEKAGILKED EHRAESYRRV | (SEQ |
| 1 | KKIIDTYHKV FIDSSLENMA KMGIENEIKA MLQSFCELYK KDHRTEGEDK | ID NO: |
| type V CRISPR- | ALDKIRAVLR GLIVGAFTGV CGRRENTVQN EKYESLFKEK LIKEILPDFV | 142) |
| associated | LSTEAESLPF SVEEATRSLK EFDSFTSYFA GFYENRKNIY STKPQSTAIA | |
| protein Cpf1 | YRLIHENLPK FIDNILVFQK IKEPIAKELE HIRADFSAGG YIKKDERLED | |
| [Porphyromonas | IFSLNYYIHV LSQAGIEKYN ALIGKIVTEG DGEMKGLNEH INLYNQQRGR | |
| crevioricanis] | EDRLPLFRPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKVLPEYKEG EPYFEKMDYK FLPDPNKMLP KVFLSKKGIE | |
| | IYKPSPKLLE QYGHGTHKKG DTFSMDDLHE LIDFFKHSIE AHEDWKQFGF | |
| | KFSDTATYEN VSSFYREVED QGYKLSFRKV SESYVYSLID QGKLYLFQIY | |
| | NKDFSPCSKG TPNLHTLYWR MLFDERNLAD VIYKLDGKAE IFFREKSLKN | |
| | DHPTHPAGKP IKKKSRQKKG EESLFEYDLV KDRRYTMDKF QFHVPITMNF | |
| | KCSAGSKVND MVNAHIREAK DMHVIGIDRG ERNLLYICVI DSRGTILDQI | |
| | SLNTINDIDY HDLLESRDKD RQQERRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGF LFYIPAWNTS NIDPTTGFVN | |
| | LFHAQYENVD KAKSFFQKFD SISYNPKKDW FEFAFDYKNF TKKAEGSRSM | |
| | WILCTHGSRI KNFRNSQKNG QWDSEEFALT EAFKSLFVRY EIDYTADLKT | |
| | AIVDEKQKDF FVDLLKLFKL TVQMRNSWKE KDLDYLISPV AGADGRFFDT | |
| | REGNKSLPKD ADANGAYNIA LKGLWALRQI RQTSEGGKLK LAISNKEWLQ | |
| | FVQERSYEKD | |
| WP_037975888. | MANSLKDFTN IYQLSKTLRF ELKPIGKTEE HINRKLIIMH DEKRGEDYKS | (SEQ |
| 1 | VTKLIDDYHR KFIHETLDPA HFDWNPLAEA LIQSGSKNNK ALPAEQKEMR | ID NO: |
| type V CRISPR- | EKIISMFTSQ AVYKKLFKKE LFSELLPEMI KSELVSDLEK QAQLDAVKSF | 143) |
| associated | DKFSTYFTGF HENRKNIYSK KDTSTSIAFR IVHQNFPKFL ANVRAYTLIK | |
| protein Cpf1 | ERAPEVIDKA QKELSGILGG KTLDDIFSIE SFNNVLTQDK IDYYNQIIGG | |
| [Synergistes | VSGKAGDKKL RGVNEFSNLY RQQHPEVASL RIKMVPLYKQ ILSDRTTLSF | |
| jonesii] | VPEALKDDEQ AINAVDGLRS ELERNDIFNR IKRLFGKNNL YSLDKIWIKN | |
| | SSISAFSNEL FKNWSFIEDA LKEFKENEFN GARSAGKKAE KWLKSKYFSF | |
| | ADIDAAVKSY SEQVSADISS APSASYFAKF TNLIETAAEN GRKFSYFAAE | |
| | SKAFRGDDGK TEIIKAYLDS LNDILHCLKP FETEDISDID TEFYSAFAEI | |
| | YDSVKDVIPV YNAVRNYTTQ KPFSTEKFKL NFENPALAKG WDKNKEQNNT | |
| | AIILMKDGKY YLGVIDKNNK LRADDLADDG SAYGYMKMNY KFIPTPHMEL | |
| | PKVFLPKRAP KRYNPSREIL LIKENKTFIK DKNFNRTDCH KLIDFFKDSI | |
| | NKHKDWRTFG FDFSDTDSYE DISDFYMEVQ DQGYKLTFTR LSAEKIDKWV | |
| | EEGRLFLFQI YNKDFADGAQ GSPNLHTLYW KAIFSEENLK DVVLKLNGEA | |
| | ELFFRRKSID KPAVHAKGSM KVNRRDIDGN PIDEGTYVEI CGYANGKRDM | |
| | ASLNAGARGL IESGLVRITE VKHELVKDKR YTIDKYFFHV PFTINFKAQG | |
| | QGNINSDVNL FLRNNKDVNI IGIDRGERNL VYVSLIDRDG HIKLQKDFNI | |
| | IGGMDYHAKL NQKEKERDTA RKSWKTIGTI KELKEGYLSQ VVHEIVRLAV | |
| | DNNAVIVMED LNIGFKRGRF KVEKQVYQKF EKMLIDKLNY LVFKDAGYDA | |
| | PCGILKGLQL TEKFESFTKL GKQCGIIFYI PAGYTSKIDP TTGFVNLFNI | |
| | NDVSSKEKQK DFIGKLDSIR FDAKRDMFTF EFDYDKFRTY QTSYRKKWAV | |
| | WTNGKRIVRE KDKDGKFRMN DRLLTEDMKN ILNKYALAYK AGEDILPDVI | |
| | SRDKSLASEI FYVFKNTLQM RNSKRDTGED FIISPVLNAK GRFFDSRKTD | |
| | AALPIDADAN GAYHIALKGS LVLDAIDEKL KEDGRIDYKD MAVSNPKWFE | |
| | FMQTRKFDF | |
| WP_081839471. | MENMANSLKD FTNIYQLSKT LRFELKPIGK TEEHINRKLI IMHDEKRGED | (SEQ |
| 1 | YKSVTKLIDD YHRKFIHETL DPAHFDWNPL AEALIQSGSK NNKALPAEQK | ID NO: |
| type V CRISPR- | EMREKIISMF TSQAVYKKLF KKELFSELLP EMIKSELVSD LEKQAQLDAV | 144) |
| associated | KSFDKFSTYF TGFHENRKNI YSKKDTSTSI AFRIVHQNFP KFLANVRAYT | |
| protein Cpf1 | LIKERAPEVI DKAQKELSGI LGGKTLDDIF SIESFNNVLT QDKIDYYNQI | |
| [Synergistes | IGGVSGKAGD KKLRGVNEFS NLYRQQHPEV ASLRIKMVPL YKQILSDRTT | |
| jonesii] | LSFVPEALKD DEQAINAVDG LRSELERNDI FNRIKRLFGK NNLYSLDKIW | |
| | IKNSSISAFS NELFKNWSFI EDALKEFKEN EFNGARSAGK KAEKWLKSKY | |
| | FSFADIDAAV KSYSEQVSAD ISSAPSASYF AKFTNLIETA AENGRKFSYF | |
| | AAESKAFRGD DGKTEIIKAY LDSLNDILHC LKPFETEDIS DIDTEFYSAF | |
| | AEIYDSVKDV IPVYNAVRNY TTQKPFSTEK FKLNFENPAL AKGWDKNKEQ | |
| | NNTAIILMKD GKYYLGVIDK NNKLRADDLA DDGSAYGYMK MNYKFIPTPH | |
| | MELPKVFLPK RAPKRYNPSR EILLIKENKT FIKDKNFNRT DCHKLIDFFK | |
| | DSINKHKDWR TFGFDFSDTD SYEDISDFYM EVQDQGYKLT FTRLSAEKID | |
| | KWVEEGRLFL FQIYNKDFAD GAQGSPNLHT LYWKAIFSEE NLKDVVLKLN | |
| | GEAELFFRRK SIDKPAVHAK GSMKVNRRDI DGNPIDEGTY VEICGYANGK | |
| | RDMASLNAGA RGLIESGLVR ITEVKHELVK DKRYTIDKYF FHVPFTINFK | |
| | AQGQGNINSD VNLFLRNNKD VNIIGIDRGE RNLVYVSLID RDGHIKLQKD | |
| | FNIIGGMDYH AKLNQKEKER DTARKSWKTI GTIKELKEGY LSQWVHEIVR | |
| | LAVDNNAVIV MEDLNIGFKR GRFKVEKQVY QKFEKMLIDK LNYLVFKDAG | |
| | YDAPCGILKG LQLTEKFESF TKLGKQCGII FYIPAGYTSK IDPTTGFVNL | |
| | FNINDVSSKE KQKDFIGKLD SIRFDAKRDM FTFEFDYDKF RTYQTSYRKK | |
| | WAVWTNGKRI VREKDKDGKF RMNDRLLTED MKNILNKYAL AYKAGEDILP | |
| | DVISRDKSLA SEIFYVFKNT LQMRNSKRDT GEDFIISPVL NAKGRFFDSR | |
| | KTDAALPIDA DANGAYHIAL KGSLVLDAID EKLKEDGRID YKDMAVSNPK | |
| | WFEFMQTRKF DF | |
| WP_006283774. | MQINNLKIIY MKFTDFTGLY SLSKTLRFEL KPIGKTLENI KKAGLLEQDQ | (SEQ |
| 1 | HRADSYKKVK KIIDEYHKAF IEKSLSNFEL KYQSEDKLDS LEEYLMYYSM | ID NO: |
| type V CRISPR- | KRIEKTEKDK FAKIQDNLRK QIADHLKGDE SYKTIFSKDL IRKNLPDFVK | 145) |
| associated | SDEERTLIKE FKDFTTYFKG FYENRENMYS AEDKSTAISH RIIHENLPKF | |
| protein Cpf1 | VDNINAFSKI ILIPELREKL NQIYQDFEEY LNVESIDEIF HLDYFSMVMT | |
| [Prevotella | QKQIEVYNAI IGGKSTNDKK IQGLNEYINL YNQKHKDCKL PKLKLLFKQI | |
| bryantii B14] | LSDRIAISWL PDNFKDDQEA LDSIDTCYKN LLNDGNVLGE GNLKLLLENI | |
| | DTYNLKGIFI RNDLQLTDIS QKMYASWNVI QDAVILDLKK QVSRKKKESA | |
| | EDYNDRLKKL YTSQESFSIQ YLNDCLRAYG KTENIQDYFA KLGAVNNEHE | |
| | QTINLFAQVR NAYTSVQAIL TTPYPENANL AQDKETVALI KNLLDSLKRL | |
| | QRFIKPLLGK GDESDKDERF YGDFTPLWET LNQITPLYNM VRNYMTRKPY | |
| | SQEKIKLNFE NSTLLGGWDL NKEHDNTAII LRKNGLYYLA IMKKSANKIF | |
| | DKDKLDNSGD CYEKMVYKLL PGANKMLPKV FFSKSRIDEF KPSENIIENY | |
| | KKGTHKKGAN FNLADCHNLI DFFKSSISKH EDWSKFNFHF SDTSSYEDLS | |
| | DFYREVEQQG YSISFCDVSV EYINKMVEKG DLYLFQIYNK DFSEFSKGTP | |
| | NMHTLYWNSL FSKENLNNII YKLNGQAEIF FRKKSLNYKR PTHPAHQAIK | |
| | NKNKCNEKKE SIFDYDLVKD KRYTVDKFQF HVPITMNFKS TGNTNINQQV | |
| | IDYLRTEDDT HIIGIDRGER HLLYLVVIDS HGKIVEQFTL NEIVNEYGGN | |
| | IYRTNYHDLL DTREQNREKA RESWQTIENI KELKEGYISQ VIHKITDLMQ | |
| | KYHAVVVLED LNMGFMRGRQ KVEKQVYQKF EEMLINKLNY LVNKKADQNS | |
| | AGGLLHAYQL TSKFESFQKL GKQSGFLFYI PAWNTSKIDP VTGFVNLFDT | |
| | RYESIDKAKA FFGKFDSIRY NADKDWFEFA FDYNNFTTKA EGTRTNWTIC | |
| | TYGSRIRTFR NQAKNSQWDN EEIDLTKAYK AFFAKHGINI YDNIKEAIAM | |
| | ETEKSFFEDL LHLLKLTLQM RNSITGTTTD YLISPVHDSK GNFYDSRICD | |
| | NSLPANADAN GAYNIARKGL MLIQQIKDST SSNRFKFSPI TNKDWLIFAQ | |
| | EKPYLND | |
| WP_024988992 | MNIKNFTGLY PLSKTLRFEL KPIGKTKENI EKNGILTKDE QRAKDYLIVK | (SEQ |
| type V CRISPR- | GFIDEYHKQF IKDRLWDFKL PLESEGEKNS LEEYQELYEL TKRNDAQEAD | ID NO: |
| associated | FTEIKDNLRS SITEQLTKSG SAYDRIFKKE FIREDLVNFL EDEKDKNIVK | 146) |
| protein Cpf1 | QFEDFTTYFT GFYENRKNMY SSEEKSTAIA YRLIHQNLPK FMDNMRSFAK | |
| [Prevotella | IANSSVSEHF SDIYESWKEY LNVNSIEEIF QLDYFSETLT QPHIEVYNYI | |
| albensis] | IGKKVLEDGT EIKGINEYVN LYNQQQKDKS KRLPFLVPLY KQILSDREKL | |
| | SWIAEEFDSD KKMLSAITES YNHLHNVLMG NENESLRNLL LNIKDYNLEK | |
| | INITNDLSLT EISQNLFGRY DVFTNGIKNK LRVLTPRKKK ETDENFEDRI | |
| | NKIFKTQKSF SIAFLNKLPQ PEMEDGKPRN IEDYFITQGA INTKSIQKED | |
| | IFAQIENAYE DAQVFLQIKD TDNKLSQNKT AVEKIKTLLD ALKELQHFIK | |
| | PLLGSGEENE KDELFYGSFL AIWDELDTIT PLYNKVRNWL TRKPYSTEKI | |
| | KLNFDNAQLL GGWDVNKEHD CAGILLRKND SYYLGIINKK TNHIFDTDIT | |
| | PSDGECYDKI DYKLLPGANK MLPKVFFSKS RIKEFEPSEA IINCYKKGTH | |
| | KKGKNFNLTD CHRLINFFKT SIEKHEDWSK FGFKFSDTET YEDISGFYRE | |
| | VEQQGYRLTS HPVSASYIHS LVKEGKLYLF QIWNKDFSQF SKGTPNLHTL | |
| | YWKMLFDKRN LSDVVYKLNG QAEVFYRKSS IEHQNRIIHP AQHPITNKNE | |
| | LNKKHTSTFK YDIIKDRRYT VDKFQFHVPI TINFKATGQN NINPIVQEVI | |
| | RQNGITHIIG IDRGERHLLY LSLIDLKGNI IKQMTLNEII NEYKGVTYKT | |
| | NYHNLLEKRE KERTEARHSW SSIESIKELK DGYMSQVIHK ITDMMVKYNA | |
| | IVVLEDLNGG FMRGRQKVEK QVYQKFEKKL IDKLNYLVDK KLDANEVGGV | |
| | LNAYQLTNKF ESFKKIGKQS GFLFYIPAWN TSKIDPITGF VNLFNTRYES | |
| | IKETKVFWSK FDIIRYNKEK NWFEFVFDYN TFTTKAEGTR TKWTLCTHGT | |
| | RIQTFRNPEK NAQWDNKEIN LTESFKALFE KYKIDITSNL KESIMQETEK | |
| | KFFQELHNLL HLTLQMRNSV TGTDIDYLIS PVADEDGNFY DSRINGKNFP | |
| | ENADANGAYN IARKGLMLIR QIKQADPQKK FKFETITNKD WLKFAQDKPY | |
| | LKD | |
| WP_039658684. | MQTLFENFTN QYPVSKTLRF ELIPQGKTKD FIEQKGLLKK DEDRAEKYKK | (SEQ |
| 1 | VKNIIDEYHK DFIEKSLNGL KLDGLEKYKT LYLKQEKDDK DKKAFDKEKE | ID NO: |
| type V CRISPR- | NLRKQIANAF RNNEKFKTLF AKELIKNDLM SFACEEDKKN VKEFEAFTTY | 147) |
| associated | FTGFHQNRAN MYVADEKRTA IASRLIHENL PKFIDNIKIF EKMKKEAPEL | |
| protein Cpf1 | LSPFNQTLKD MKDVIKGTTL EEIFSLDYFN KTLTQSGIDI YNSVIGGRTP | |
| [Smithella sp. | EEGKTKIKGL NEYINTDFNQ KQTDKKKRQP KFKQLYKQIL SDRQSLSFIA | |
| SC_K08D17] | EAFKNDTEIL EAIEKFYVNE LLHFSNEGKS TNVLDAIKNA VSNLESFNLT | |
| | KMYFRSGASL TDVSRKVFGE WSIINRALDN YYATTYPIKP REKSEKYEER | |
| | KEKWLKQDFN VSLIQTAIDE YDNETVKGKN SGKVIADYFA KFCDDKETDL | |
| | IQKVNEGYIA VKDLLNTPCP ENEKLGSNKD QVKQIKAFMD SIMDIMHFVR | |
| | PLSLKDTDKE KDETFYSLFT PLYDHLTQTI ALYNKVRNYL TQKPYSTEKI | |
| | KLNFENSTLL GGWDLNKETD NTAIILRKDN LYYLGIMDKR HNRIFRNVPK | |
| | ADKKDFCYEK MVYKLLPGAN KMLPKVFFSQ SRIQEFTPSA KLLENYANET | |
| | HKKGDNFNLN HCHKLIDFFK DSINKHEDWK NFDFRFSATS TYADLSGFYH | |
| | EVEHQGYKIS FQSVADSFID DLVNEGKLYL FQIYNKDFSP FSKGKPNLHT | |
| | LYWKMLFDEN NLKDVVYKLN GEAEVFYRKK SIAEKNTTIH KANESIINKN | |
| | PDNPKATSTF NYDIVKDKRY TIDKFQFHIP ITMNFKAEGI FNMNQRVNQF | |
| | LKANPDINII GIDRGERHLL YYALINQKGK ILKQDTLNVI ANEKQKVDYH | |
| | NLLDKKEGDR ATARQEWGVI ETIKELKEGY LSQVIHKLTD LMIENNAIIV | |
| | MEDLNFGFKR GRQKVEKQVY QKFEKMLIDK LNYLVDKNKK ANELGGLLNA | |
| | FQLANKFESF QKMGKQNGFI FYVPAWNTSK TDPATGFIDF LKPRYENLNQ | |
| | AKDFFEKFDS IRLNSKADYF EFAFDFKNFT EKADGGRTKW TVCTTNEDRY | |
| | AWNRALNNNR GSQEKYDITA ELKSLFDGKV DYKSGKDLKQ QIASQESADF | |
| | FKALMKNLSI TLSLRHNNGE KGDNEQDYIL SPVADSKGRF FDSRKADDDM | |
| | PKNADANGAY HIALKGLWCL EQISKTDDLK KVKLAISNKE WLEFVQTLKG | |
| WP_037385181 | MQTLFENFTN QYPVSKTLRF ELIPQGKTKD FIEQKGLLKK DEDRAEKYKK | (SEQ |
| type V CRISPR- | VKNIIDEYHK DFIEKSLNGL KLDGLEEYKT LYLKQEKDDK DKKAFDKEKE | ID NO: |
| associated | NLRKQIANAF RNNEKFKTLF AKELIKNDLM SFACEEDKKN VKEFEAFTTY | 148) |
| protein Cpf1 | FTGFHQNRAN MYVADEKRTA IASRLIHENL PKFIDNIKIF EKMKKEAPEL | |
| [Smithella sp. | LSPFNQTLKD MKDVIKGTTL EEIFSLDYFN KTLTQSGIDI YNSVIGGRTP | |
| SCADC] | EEGKTKIKGL NEYINTDFNQ KQTDKKKRQP KFKQLYKQIL SDRQSLSFIA | |
| | EAFKNDTEIL EAIEKFYVNE LLHFSNEGKS TNVLDAIKNA VSNLESFNLT | |
| | KIYFRSGTSL TDVSRKVFGE WSIINRALDN YYATTYPIKP REKSEKYEER | |
| | KEKWLKQDFN VSLIQTAIDE YDNETVKGKN SGKVIVDYFA KFCDDKETDL | |
| | IQKVNEGYIA VKDLLNTPYP ENEKLGSNKD QVKQIKAFMD SIMDIMHFVR | |
| | PLSLKDTDKE KDETFYSLFT PLYDHLTQTI ALYNKVRNYL TQKPYSTEKI | |
| | KLNFENSTLL GGWDLNKETD NTAIILRKEN LYYLGIMDKR HNRIFRNVPK | |
| | ADKKDSCYEK MVYKLLPGAN KMLPKVFFSQ SRIQEFTPSA KLLENYENET | |
| | HKKGDNFNLN HCHQLIDFFK DSINKHEDWK NFDFRFSATS TYADLSGFYH | |
| | EVEHQGYKIS FQSIADSFID DLVNEGKLYL FQIYNKDFSP FSKGKPNLHT | |
| | LYWKMLFDEN NLKDVVYKLN GEAEVFYRKK SIAEKNTTIH KANESIINKN | |
| | PDNPKATSTF NYDIVKDKRY TIDKFQFHVP ITMNFKAEGI FNMNQRVNQF | |
| | LKANPDINII GIDRGERHLL YYTLINQKGK ILKQDTLNVI ANEKQKVDYH | |
| | NLLDKKEGDR ATARQEWGVI ETIKELKEGY LSQVIHKLTD LMIENNAIIV | |
| | MEDLNFGFKR GRQKVEKQVY QKFEKMLIDK LNYLVDKNKK ANELGGLLNA | |
| | FQLANKFESF QKMGKQNGFI FYVPAWNTSK TDPATGFIDF LKPRYENLKQ | |
| | AKDFFEKFDS IRLNSKADYF EFAFDFKNFT GKADGGRTKW TVCTTNEDRY | |
| | AWNRALNNNR GSQEKYDITA ELKSLFDGKV DYKSGKDLKQ QIASQELADF | |
| | FRTLMKYLSV TLSLRHNNGE KGETEQDYIL SPVADSMGKF FDSRKAGDDM | |
| | PKNADANGAY HIALKGLWCL EQISKTDDLK KVKLAISNKE WLEFMQTLKG | |
| WP_039871282.1 | MKFTDFTGLY SLSKTLRFEL KPIGKTLENI KKAGLLEQDQ HRADSYKKVK | (SEQ |
| type V CRISPR- | KIIDEYHKAF IEKSLSNFEL KYQSEDKLDS LEEYLMYYSM KRIEKTEKDK | ID NO: |
| associated | FAKIQDNLRK QIADHLKGDE SYKTIFSKDL IRKNLPDFVK SDEERTLIKE | 149) |
| protein Cpf1 | FKDFTTYFKG FYENRENMYS AEDKSTAISH RIIHENLPKF VDNINAFSKI | |
| [Prevotella | ILIPELREKL NQIYQDFEEY LNVESIDEIF HLDYFSMVMT QKQIEVYNAI | |
| bryantii B14] | IGGKSTNDKK IQGLNEYINL YNQKHKDCKL PKLKLLFKQI LSDRIAISWL | |
| | PDNFKDDQEA LDSIDTCYKN LLNDGNVLGE GNLKLLLENI DTYNLKGIFI | |
| | RNDLQLTDIS QKMYASWNVI QDAVILDLKK QVSRKKKESA EDYNDRLKKL | |
| | YTSQESFSIQ YLNDCLRAYG KTENIQDYFA KLGAVNNEHE QTINLFAQVR | |
| | NAYTSVQAIL TTPYPENANL AQDKETVALI KNLLDSLKRL QRFIKPLLGK | |
| | GDESDKDERF YGDFTPLWET LNQITPLYNM VRNYMTRKPY SQEKIKLNFE | |
| | NSTLLGGWDL NKEHDNTAII LRKNGLYYLA IMKKSANKIF DKDKLDNSGD | |
| | CYEKMVYKLL PGANKMLPKV FFSKSRIDEF KPSENIIENY KKGTHKKGAN | |
| | FNLADCHNLI DFFKSSISKH EDWSKFNFHF SDTSSYEDLS DFYREVEQQG | |
| | YSISFCDVSV EYINKMVEKG DLYLFQIYNK DFSEFSKGTP NMHTLYWNSL | |
| | FSKENLNNII YKLNGQAEIF FRKKSLNYKR PTHPAHQAIK NKNKCNEKKE | |
| | SIFDYDLVKD KRYTVDKFQF HVPITMNFKS TGNTNINQQV IDYLRTEDDT | |
| | HIIGIDRGER HLLYLVVIDS HGKIVEQFTL NEIVNEYGGN IYRTNYHDLL | |
| | DTREQNREKA RESWQTIENI KELKEGYISQ VIHKITDLMQ KYHAVVVLED | |
| | LNMGFMRGRQ KVEKQVYQKF EEMLINKLNY LVNKKADQNS AGGLLHAYQL | |
| | TSKFESFQKL GKQSGFLFYI PAWNTSKIDP VTGFVNLFDT RYESIDKAKA | |
| | FFGKFDSIRY NADKDWFEFA FDYNNFTTKA EGTRTNWTIC TYGSRIRTFR | |
| | NQAKNSQWDN EEIDLTKAYK AFFAKHGINI YDNIKEAIAM ETEKSFFEDL | |
| | LHLLKLTLQM RNSITGTTTD YLISPVHDSK GNFYDSRICD NSLPANADAN | |
| | GAYNIARKGL MLIQQIKDST SSNRFKFSPI TNKDWLIFAQ EKPYLND | |
| EKE28449.1 | MFKGDAFTGL YEVQKTLRFE LVPIGLTQSY LENDWVIQKD KEVEENYGKI | (SEQ |
| hypothetical | KAYFDLIHKE FVRQSLENAW LCQLDDFYEK YIELHNSLET RKDKNLAKQF | ID NO: |
| protein | EKVMKSLKKE FVSFFDAKWN EWKQKFSFLK KWWIDVLNEK EVLDLMAEFY | 150) |
| ACD_3C00058G | PDEKELFDKF DKFFTYFSNF KESRKNFYAD DGRAWAIATR AIDENLITFI | |
| 0015 [uncultured | KNIEDFKKLN SSFREFVNDN FSEEDKQIFE IDFYNNCLLQ PWIDKYNKIV | |
| bacterium (gcode | WWYSLENWEK VQWLNEKINN FKQNQNKSNS KDLKFPRMKL LYKQILGDKE | |
| 4)] | KKVYIDEIRD DKNLIDLIDN SKRRNQIKID NANDIINDFI NNNAKFELDK | |
| | IYLTRQSINT ISSKYFSSWD YIRWYFWTGE LQEFVSFYDL KETFWKIEYE | |
| | TLENIFKDCY VKGINTESQN NIVFETQGIY ENFLNIFKFE FNQNISQISL | |
| | LEWELDKIQN EDIKKNEKQV EVIKNYFDSV MSVYKMTKYF SLEKWKKRVE | |
| | LDTDNNFYND FNEYLEGFEI WKDYNLVRNY ITKKQVNTDK IKLNFDNSQF | |
| | LTWWDKDKEN ERLGIILRRE WKYYLWILKK WNTLNFGDYL QKEWEIFYEK | |
| | MNYKQLNNVY RQLPRLLFPL TKKLNELKWD ELKKYLSKYI QNFWYNEEIA | |
| | QIKIEFDIFQ ESKEKWEKFD IDKLRKLIEY YKKWVLALYS DLYDLEFIKY | |
| | KNYDDLSIFY SDVEKKMYNL NFTKIDKSLI DGKVKSWELY LFQIYNKDFS | |
| | ESKKEWSTEN IHTKYFKLLF NEKNLQNLVV KLSWWADIFF RDKTENLKFK | |
| | KDKNGQEILD HRRFSQDKIM FHISITLNAN CWDKYWFNQY VNEYMNKERD | |
| | IKIIWIDRWE KHLAYYCVID KSWKIFNNEI WTLNELNWVN YLEKLEKIES | |
| | SRKDSRISWW EIENIKELKN GYISQVINKL TELIVKYNAI IVFEDLNIWF | |
| | KRWRQKIEKQ IYQKLELALA KKLNYLTQKD KKDDEILWNL KALQLVPKVN | |
| | DYQDIWNYKQ SWIMFYVRAN YTSVTCPNCW LRKNLYISNS ATKENQKKSL | |
| | NSIAIKYNDW KFSFSYEIDD KSWKQKQSLN KKKFIVYSDI ERFVYSPLEK | |
| | LTKVIDVNKK LLELFRDFNL SLDINKQIQE KDLDSVFFKS LTHLFNLILQ | |
| | LRNSDSKDNK DYISCPSCYY HSNNWLQWFE FNWDANWAYN IARKGIILLD | |
| | RIRKNQEKPD LYVSDIDWDN FVQSNQFPNT IIPIQNIEKQ VPLNIKI | |
| WP_018359861. | MKTQHFFEDF TSLYSLSKTI RFELKPIGKT LENIKKNGLI RRDEQRLDDY | (SEQ |
| 1 | EKLKKVIDEY HEDFIANILS SFSFSEEILQ SYIQNLSESE ARAKIEKTMR | ID NO: |
| type V CRISPR- | DTLAKAFSED ERYKSIFKKE LVKKDIPVWC PAYKSLCKKF DNFTTSLVPF | 151) |
| associated | HENRKNLYTS NEITASIPYR IVHVNLPKFI QNIEALCELQ KKMGADLYLE | |
| protein Cpf1 | MMENLRNVWP SFVKTPDDLC NLKTYNHLMV QSSISEYNRF VGGYSTEDGT | |
| [Porphyromonas | KHQGINEWIN IYRQRNKEMR LPGLVFLHKQ ILAKVDSSSF ISDTLENDDQ | |
| macacae] | VFCVLRQFRK LFWNTVSSKE DDAASLKDLF CGLSGYDPEA IYVSDAHLAT | |
| | ISKNIFDRWN YISDAIRRKT EVLMPRKKES VERYAEKISK QIKKRQSYSL | |
| | AELDDLLAHY SEESLPAGFS LLSYFTSLGG QKYLVSDGEV ILYEEGSNIW | |
| | DEVLIAFRDL QVILDKDFTE KKLGKDEEAV SVIKKALDSA LRLRKFFDLL | |
| | SGTGAEIRRD SSFYALYTDR MDKLKGLLKM YDKVRNYLTK KPYSIEKFKL | |
| | HFDNPSLLSG WDKNKELNNL SVIFRQNGYY YLGIMTPKGK NLFKTLPKLG | |
| | AEEMFYEKME YKQIAEPMLM LPKVFFPKKT KPAFAPDQSV VDIYNKKTFK | |
| | TGQKGFNKKD LYRLIDFYKE ALTVHEWKLF NFSFSPTEQY RNIGEFFDEV | |
| | REQAYKVSMV NVPASYIDEA VENGKLYLFQ IYNKDFSPYS KGIPNLHTLY | |
| | WKALFSEQNQ SRVYKLCGGG ELFYRKASLH MQDTTVHPKG ISIHKKNLNK | |
| | KGETSLFNYD LVKDKRFTED KFFFHVPISI NYKNKKITNV NQMVRDYIAQ | |
| | NDDLQIIGID RGERNLLYIS RIDTRGNLLE QFSLNVIESD KGDLRTDYQK | |
| | ILGDREQERL RRRQEWKSIE SIKDLKDGYM SQVVHKICNM VVEHKAIVVL | |
| | ENLNLSFMKG RKKVEKSVYE KFERMLVDKL NYLVVDKKNL SNEPGGLYAA | |
| | YQLTNPLFSF EELHRYPQSG ILFFVDPWNT SLTDPSTGFV NLLGRINYTN | |
| | VGDARKFFDR FNAIRYDGKG NILFDLDLSR FDVRVETQRK LWTLTTFGSR | |
| | IAKSKKSGKW MVERIENLSL CFLELFEQFN IGYRVEKDLK KAILSQDRKE | |
| | FYVRLIYLFN LMMQIRNSDG EEDYILSPAL NEKNLQFDSR LIEAKDLPVD | |
| | ADANGAYNVA RKGLMVVQRI KRGDHESIHR IGRAQWLRYV QEGIVE | |
| WP_013282991 | MLLYENYTKR NQITKSLRLE LRPQGKTLRN IKELNLLEQD KAIYALLERL | (SEQ |
| type V CRISPR- | KPVIDEGIKD IARDTLKNCE LSFEKLYEHF LSGDKKAYAK ESERLKKEIV | ID NO: |
| associated | KTLIKNLPEG IGKISEINSA KYLNGVLYDF IDKTHKDSEE KQNILSDILE | 152) |
| protein Cpf1 | TKGYLALFSK FLTSRITTLE QSMPKRVIEN FEIYAANIPK MQDALERGAV | |
| [Butyrivibrio | SFAIEYESIC SVDYYNQILS QEDIDSYNRL ISGIMDEDGA KEKGINQTIS | |
| proteoclasticus] | EKNIKIKSEH LEEKPFRILK QLHKQILEER EKAFTIDHID SDEEVVQVTK | |
| | EAFEQTKEQW ENIKKINGFY AKDPGDITLF IVVGPNQTHV LSQLIYGEHD | |
| | RIRLLLEEYE KNTLEVLPRR TKSEKARYDK FVNAVPKKVA KESHTFDGLQ | |
| | KMTGDDRLFI LYRDELARNY MRIKEAYGTF ERDILKSRRG IKGNRDVQES | |
| | LVSFYDELTK FRSALRIINS GNDEKADPIF YNTFDGIFEK ANRTYKAENL | |
| | CRNYVTKSPA DDARIMASCL GTPARLRTHW WNGEENFAIN DVAMIRRGDE | |
| | YYYFVLTPDV KPVDLKTKDE TDAQIFVQRK GAKSFLGLPK ALFKCILEPY | |
| | FESPEHKNDK NCVIEEYVSK PLTIDRRAYD IFKNGTFKKT NIGIDGLTEE | |
| | KFKDDCRYLI DVYKEFIAVY TRYSCFNMSG LKRADEYNDI GEFFSDVDTR | |
| | LCTMEWIPVS FERINDMVDK KEGLLFLVRS MFLYNRPRKP YERTFIQLFS | |
| | DSNMEHTSML LNSRAMIQYR AASLPRRVTH KKGSILVALR DSNGEHIPMH | |
| | IREAIYKMKN NFDISSEDFI MAKAYLAEHD VAIKKANEDI IRNRRYTEDK | |
| | FFLSLSYTKN ADISARTLDY INDKVEEDTQ DSRMAVIVTR NLKDLTYVAV | |
| | VDEKNNVLEE KSLNEIDGVN YRELLKERTK IKYHDKTRLW QYDVSSKGLK | |
| | EAYVELAVTQ ISKLATKYNA VVVVESMSST FKDKFSFLDE QIFKAFEARL | |
| | CARMSDLSFN TIKEGEAGSI SNPIQVSNNN GNSYQDGVIY FLNNAYTRTL | |
| | CPDTGFVDVF DKTRLITMQS KRQFFAKMKD IRIDDGEMLF TFNLEEYPTK | |
| | RLLDRKEWTV KIAGDGSYFD KDKGEYVYVN DIVREQIIPA LLEDKAVFDG | |
| | NMAEKFLDKT AISGKSVELI YKWFANALYG IITKKDGEKI YRSPITGTEI | |
| | DVSKNTTYNF GKKFMFKQEY RGDGDFLDAF LNYMQAQDIA V | |
| WP_048112740. | MNNYDEFTKL YPIQKTIRFE LKPQGRTMEH LETFNFFEED RDRAEKYKIL | (SEQ |
| 1 | KEAIDEYHKK FIDEHLTNMS LDWNSLKQIS EKYYKSREEK DKKVFLSEQK | ID NO: |
| type V CRISPR- | RMRQEIVSEF KKDDRFKDLF SKKLFSELLK EEIYKKGNHQ EIDALKSFDK | 153) |
| associated | FSGYFIGLHE NRKNMYSDGD EITAISNRIV NENFPKFLDN LQKYQEARKK | |
| protein Cpf1 | YPEWIIKAES ALVAHNIKMD EVFSLEYFNK VLNQEGIQRY NLALGGYVTK | |
| [Candidatus | SGEKMMGLND ALNLAHQSEK SSKGRIHMTP LFKQILSEKE SFSYIPDVFT | |
| Methanoplasma | EDSQLLPSIG GFFAQIENDK DGNIFDRALE LISSYAEYDT ERIYIRQADI | |
| termitum] | NRVSNVIFGE WGTLGGLMRE YKADSINDIN LERTCKKVDK WLDSKEFALS | |
| | DVLEAIKRTG NNDAFNEYIS KMRTAREKID AARKEMKFIS EKISGDEESI | |
| | HIIKTLLDSV QQFLHFFNLF KARQDIPLDG AFYAEFDEVH SKLFAIVPLY | |
| | NKVRNYLTKN NLNTKKIKLN FKNPTLANGW DQNKVYDYAS LIFLRDGNYY | |
| | LGIINPKRKK NIKFEQGSGN GPFYRKMVYK QIPGPNKNLP RVFLTSTKGK | |
| | KEYKPSKEII EGYEADKHIR GDKFDLDFCH KLIDFFKESI EKHKDWSKFN | |
| | FYFSPTESYG DISEFYLDVE KQGYRMHFEN ISAETIDEYV EKGDLFLFQI | |
| | YNKDFVKAAT GKKDMHTIYW NAAFSPENLQ DVVVKLNGEA ELFYRDKSDI | |
| | KEIVHREGEI LVNRTYNGRT PVPDKIHKKL TDYHNGRTKD LGEAKEYLDK | |
| | VRYFKAHYDI TKDRRYLNDK IYFHVPLTLN FKANGKKNLN KMVIEKFLSD | |
| | EKAHIIGIDR GERNLLYYSI IDRSGKIIDQ QSLNVIDGFD YREKLNQREI | |
| | EMKDARQSWN AIGKIKDLKE GYLSKAVHEI TKMAIQYNAI VVMEELNYGF | |
| | KRGRFKVEKQ IYQKFENMLI DKMNYLVFKD APDESPGGVL NAYQLTNPLE | |
| | SFAKLGKQTG ILFYVPAAYT SKIDPTTGFV NLFNTSSKTN AQERKEFLQK | |
| | FESISYSAKD GGIFAFAFDY RKFGTSKTDH KNVWTAYTNG ERMRYIKEKK | |
| | RNELFDPSKE IKEALTSSGI KYDGGQNILP DILRSNNNGL IYTMYSSFIA | |
| | AIQMRVYDGK EDYIISPIKN SKGEFFRTDP KRRELPIDAD ANGAYNIALR | |
| | GELTMRAIAE KFDPDSEKMA KLELKHKDWF EFMQTRGD | |
| WP_027407524. | MVAFIDEFVG QYPVSKTLRF EARPVPETKK WLESDQCSVL FNDQKRNEYY | (SEQ |
| 1 | GVLKELLDDY YRAYIEDALT SFTLDKALLE NAYDLYCNRD TNAFSSCCEK | ID NO: |
| type V CRISPR- | LRKDLVKAFG NLKDYLLGSD QLKDLVKLKA KVDAPAGKGK KKIEVDSRLI | 154) |
| associated | NWLNNNAKYS AEDREKYIKA IESFEGFVTY LTNYKQAREN MESSEDKSTA | |
| protein Cpf1 | IAFRVIDQNM VTYFGNIRIY EKIKAKYPEL YSALKGFEKF FSPTAYSEIL | |
| [Anaerovibrio sp. | SQSKIDEYNY QCIGRPIDDA DFKGVNSLIN EYRQKNGIKA RELPVMSMLY | |
| RM50] | KQILSDRDNS FMSEVINRNE EAIECAKNGY KVSYALFNEL LQLYKKIFTE | |
| | DNYGNIYVKT QPLTELSQAL FGDWSILRNA LDNGKYDKDI INLAELEKYF | |
| | SEYCKVLDAD DAAKIQDKFN LKDYFIQKNA LDATLPDLDK ITQYKPHLDA | |
| | MLQAIRKYKL FSMYNGRKKM DVPENGIDFS NEFNAIYDKL SEFSILYDRI | |
| | RNFATKKPYS DEKMKLSFNM PTMLAGWDYN NETANGCFLF IKDGKYFLGV | |
| | ADSKSKNIFD FKKNPHLLDK YSSKDIYYKV KYKQVSGSAK MLPKVVFAGS | |
| | NEKIFGHLIS KRILEIREKK LYTAAAGDRK AVAEWIDFMK SAIAIHPEWN | |
| | EYFKFKFKNT AEYDNANKFY EDIDKQTYSL EKVEIPTEYI DEMVSQHKLY | |
| | LFQLYTKDFS DKKKKKGTDN LHTMYWHGVF SDENLKAVTE GTQPIIKLNG | |
| | EAEMFMRNPS IEFQVTHEHN KPIANKNPLN TKKESVFNYD LIKDKRYTER | |
| | KFYFHCPITL NFRADKPIKY NEKINRFVEN NPDVCIIGID RGERHLLYYT | |
| | VINQTGDILE QGSLNKISGS YTNDKGEKVN KETDYHDLLD RKEKGKHVAQ | |
| | QAWETIENIK ELKAGYLSQV VYKLTQLMLQ YNAVIVLENL NVGFKRGRTK | |
| | VEKQVYQKFE KAMIDKLNYL VFKDRGYEMN GSYAKGLQLT DKFESFDKIG | |
| | KQTGCIYYVI PSYTSHIDPK TGFVNLLNAK LRYENITKAQ DTIRKFDSIS | |
| | YNAKADYFEF AFDYRSFGVD MARNEWVVCT CGDLRWEYSA KTRETKAYSV | |
| | TDRLKELFKA HGIDYVGGEN LVSHITEVAD KHELSTLLFY LRLVLKMRYT | |
| | VSGTENENDF ILSPVEYAPG KFFDSREATS TEPMNADANG AYHIALKGLM | |
| | TIRGIEDGKL HNYGKGGENA AWFKFMQNQE YKNNG | |
| WP_044910712. | MDYGNGQFER RAPLTKTITL RLKPIGETRE TIREQKLLEQ DAAFRKLVET | (SEQ |
| 1 | VTPIVDDCIR KIADNALCHF GTEYDFSCLG NAISKNDSKA IKKETEKVEK | ID NO: |
| type V CRISPR- | LLAKVLTENL PDGLRKVNDI NSAAFIQDTL TSFVQDDADK RVLIQELKGK | 155) |
| associated | TVLMQRFLTT RITALTVWLP DRVFENFNIF IENAEKMRIL LDSPLNEKIM | |
| protein Cpf1 | KFDPDAEQYA SLEFYGQCLS QKDIDSYNLI ISGIYADDEV KNPGINEIVK | |
| [Lachnospiraceae | EYNQQIRGDK DESPLPKLKK LHKQILMPVE KAFFVRVLSN DSDARSILEK | |
| bacterium | ILKDTEMLPS KIIEAMKEAD AGDIAVYGSR LHELSHVIYG DHGKLSQIIY | |
| MC2017] | DKESKRISEL METLSPKERK ESKKRLEGLE EHIRKSTYTF DELNRYAEKN | |
| | VMAAYIAAVE ESCAEIMRKE KDLRTLLSKE DVKIRGNRHN TLIVKNYFNA | |
| | WTVFRNLIRI LRRKSEAEID SDFYDVLDDS VEVLSLTYKG ENLCRSYITK | |
| | KIGSDLKPEI ATYGSALRPN SRWWSPGEKF NVKFHTIVRR DGRLYYFILP | |
| | KGAKPVELED MDGDIECLQM RKIPNPTIFL PKLVFKDPEA FFRDNPEADE | |
| | FVFLSGMKAP VTITRETYEA YRYKLYTVGK LRDGEVSEEE YKRALLQVLT | |
| | AYKEFLENRM IYADLNFGFK DLEEYKDSSE FIKQVETHNT FMCWAKVSSS | |
| | QLDDLVKSGN GLLFEIWSER LESYYKYGNE KVLRGYEGVL LSILKDENLV | |
| | SMRTLLNSRP MLVYRPKESS KPMVVHRDGS RVVDRFDKDG KYIPPEVHDE | |
| | LYRFFNNLLI KEKLGEKARK ILDNKKVKVK VLESERVKWS KFYDEQFAVT | |
| | FSVKKNADCL DTTKDLNAEV MEQYSESNRL ILIRNTTDIL YYLVLDKNGK | |
| | VLKQRSLNII NDGARDVDWK ERFRQVTKDR NEGYNEWDYS RTSNDLKEVY | |
| | LNYALKEIAE AVIEYNAILI IEKMSNAFKD KYSFLDDVTF KGFETKLLAK | |
| | LSDLHFRGIK DGEPCSFTNP LQLCQNDSNK ILQDGVIFMV PNSMTRSLDP | |
| | DTGFIFAIND HNIRTKKAKL NFLSKFDQLK VSSEGCLIMK YSGDSLPTHN | |
| | TDNRVWNCCC NHPITNYDRE TKKVEFIEEP VEELSRVLEE NGIETDTELN | |
| | KLNERENVPG KVVDAIYSLV LNYLRGTVSG VAGQRAVYYS PVTGKKYDIS | |
| | FIQAMNLNRK CDYYRIGSKE RGEWTDFVAQ LIN | |
| WP_081834226 | MTMDYGNGQF ERRAPLTKTI TLRLKPIGET RETIREQKLL EQDAAFRKLV | (SEQ |
| type V CRISPR- | ETVTPIVDDC IRKIADNALC HFGTEYDFSC LGNAISKNDS KAIKKETEKV | ID NO: |
| associated | EKLLAKVLTE NLPDGLRKVN DINSAAFIQD TLTSFVQDDA DKRVLIQELK | 156) |
| protein Cpf1 | GKTVLMQRFL TTRITALTVW LPDRVFENFN IFIENAEKMR ILLDSPLNEK | |
| [Lachnospiracea | IMKFDPDAEQ YASLEFYGQC LSQKDIDSYN LIISGIYADD EVKNPGINEI | |
| e bacterium | VKEYNQQIRG DKDESPLPKL KKLHKQILMP VEKAFFVRVL SNDSDARSIL | |
| MC2017]. | EKILKDTEML PSKIIEAMKE ADAGDIAVYG SRLHELSHVI YGDHGKLSQI | |
| | IYDKESKRIS ELMETLSPKE RKESKKRLEG LEEHIRKSTY TFDELNRYAE | |
| | KNVMAAYIAA VEESCAEIMR KEKDLRTLLS KEDVKIRGNR HNTLIVKNYF | |
| | NAWTVFRNLI RILRRKSEAE IDSDFYDVLD DSVEVLSLTY KGENLCRSYI | |
| | TKKIGSDLKP EIATYGSALR PNSRWWSPGE KFNVKFHTIV RRDGRLYYFI | |
| | LPKGAKPVEL EDMDGDIECL QMRKIPNPTI FLPKLVFKDP EAFFRDNPEA | |
| | DEFVFLSGMK APVTITRETY EAYRYKLYTV GKLRDGEVSE EEYKRALLQV | |
| | LTAYKEFLEN RMIYADLNFG FKDLEEYKDS SEFIKQVETH NTFMCWAKVS | |
| | SSQLDDLVKS GNGLLFEIWS ERLESYYKYG NEKVLRGYEG VLLSILKDEN | |
| | LVSMRTLLNS RPMLVYRPKE SSKPMVVHRD GSRVVDRFDK DGKYIPPEVH | |
| | DELYRFFNNL LIKEKLGEKA RKILDNKKVK VKVLESERVK WSKFYDEQFA | |
| | VTFSVKKNAD CLDTTKDLNA EVMEQYSESN RLILIRNTTD ILYYLVLDKN | |
| | GKVLKQRSLN IINDGARDVD WKERFRQVTK DRNEGYNEWD YSRTSNDLKE | |
| | VYLNYALKEI AEAVIEYNAI LIIEKMSNAF KDKYSFLDDV TFKGFETKLL | |
| | AKLSDLHFRG IKDGEPCSFT NPLQLCQNDS NKILQDGVIF MVPNSMTRSL | |
| | DPDTGFIFAI NDHNIRTKKA KLNFLSKFDQ LKVSSEGCLI MKYSGDSLPT | |
| | HNTDNRVWNC CCNHPITNYD RETKKVEFIE EPVEELSRVL EENGIETDTE | |
| | LNKLNERENV PGKVVDAIYS LVLNYLRGTV SGVAGQRAVY YSPVTGKKYD | |
| | ISFIQAMNLN RKCDYYRIGS KERGEWTDFV AQLIN | |
| WP_027216152.1 | MYYESLTKLY PIKKTIRNEL VPIGKTLENI KKNNILEADE DRKIAYIRVK | (SEQ |
| type V CRISPR- | AIMDDYHKRL INEALSGFAL IDLDKAANLY LSRSKSADDI ESFSRFQDKL | ID NO: |
| associated | RKAIAKRLRE HENFGKIGNK DIIPLLQKLS ENEDDYNALE SFKNFYTYFE | 157) |
| protein Cpf1 | SYNDVRLNLY SDKEKSSTVA YRLINENLPR FLDNIRAYDA VQKAGITSEE | |
| [Butyrivibrio | LSSEAQDGLF LVNTFNNVLI QDGINTYNED IGKLNVAINL YNQKNASVQG | |
| fibrisolvens] | FRKVPKMKVL YKQILSDREE SFIDEFESDT ELLDSLESHY ANLAKYFGSN | |
| | KVQLLFTALR ESKGVNVYVK NDIAKTSFSN VVFGSWSRID ELINGEYDDN | |
| | NNRKKDEKYY DKRQKELKKN KSYTIEKIIT LSTEDVDVIG KYIEKLESDI | |
| | DDIRFKGKNF YEAVLCGHDR SKKLSKNKGA VEAIKGYLDS VKDFERDLKL | |
| | INGSGQELEK NLVVYGEQEA VLSELSGIDS LYNMTRNYLT KKPFSTEKIK | |
| | LNFNKPTFLD GWDYGNEEAY LGFFMIKEGN YFLAVMDANW NKEFRNIPSV | |
| | DKSDCYKKVI YKQISSPEKS IQNLMVIDGK TVKKNGRKEK EGIHSGENLI | |
| | LEELKNTYLP KKINDIRKRR SYLNGDTFSK KDLTEFIGYY KQRVIEYYNG | |
| | YSFYFKSDDD YASFKEFQED VGRQAYQISY VDVPVSFVDD LINSGKLYLF | |
| | RVYNKDFSEY SKGRLNLHTL YFKMLFDERN LKNVVYKLNG QAEVFYRPSS | |
| | IKKEELIVHR AGEEIKNKNP KRAAQKPTRR LDYDIVKDRR YSQDKFMLHT | |
| | SIIMNFGAEE NVSFNDIVNG VLRNEDKVNV IGIDRGERNL LYVWVIDPEG | |
| | KILEQRSLNC ITDSNLDIET DYHRLLDEKE SDRKIARRDW TTIENIKELK | |
| | AGYLSQWVHI VAELVLKYNA IICLEDLNFG FKRGRQKVEK QVYQKFEKML | |
| | IDKLNYLVMD KSREQLSPEK ISGALNALQL TPDFKSFKVL GKQTGIIYYV | |
| | PAYLTSKIDP MTGFANLFYV KYENVDKAKE FFSKFDSIKY NKDGKNWNTK | |
| | GYFEFAFDYK KFTDRAYGRV SEWTVCTVGE RIIKFKNKEK NNSYDDKVID | |
| | LTNSLKELFD SYKVTYESEV DLKDAILAID DPAFYRDLTR RLQQTLQMRN | |
| | SSCDGSRDYI ISPVKNSKGE FFCSDNNDDT TPNDADANGA FNIARKGLWV | |
| | LNEIRNSEEG SKINLAMSNA QWLEYAQDNT I | |
| WP_016301126. | MHENNGKIAD NFIGIYPVSK TLRFELKPVG KTQEYIEKHG ILDEDLKRAG | (SEQ |
| 1 | DYKSVKKIID AYHKYFIDEA LNGIQLDGLK NYYELYEKKR DNNEEKEFQK | ID NO: |
| type V CRISPR- | IQMSLRKQIV KRFSEHPQYK YLFKKELIKN VLPEFTKDNA EEQTLVKSFQ | 158) |
| associated | EFTTYFEGFH QNRKNMYSDE EKSTAIAYRV VHQNLPKYID NMRIFSMILN | |
| protein Cpf1 | TDIRSDLTEL FNNLKTKMDI TIVEEYFAID GFNKVVNQKG IDVYNTILGA | |
| [Lachnospiraceae | FSTDDNTKIK GLNEYINLYN QKNKAKLPKL KPLFKQILSD RDKISFIPEQ | |
| bacterium | FDSDTEVLEA VDMFYNRLLQ FVIENEGQIT ISKLLTNFSA YDLNKIYVKN | |
| COE1] | DTTISAISND LFDDWSYISK AVRENYDSEN VDKNKRAAAY EEKKEKALSK | |
| | IKMYSIEELN FFVKKYSCNE CHIEGYFERR ILEILDKMRY AYESCKILHD | |
| | KGLINNISLC QDRQAISELK DFLDSIKEVQ WLLKPLMIGQ EQADKEEAFY | |
| | TELLRIWEEL EPITLLYNKV RNYVTKKPYT LEKVKLNFYK STLLDGWDKN | |
| | KEKDNLGIIL LKDGQYYLGI MNRRNNKIAD DAPLAKTDNV YRKMEYKLLT | |
| | KVSANLPRIF LKDKYNPSEE MLEKYEKGTH LKGENFCIDD CRELIDFFKK | |
| | GIKQYEDWGQ FDFKFSDTES YDDISAFYKE VEHQGYKITF RDIDETYIDS | |
| | LVNEGKLYLF QIYNKDFSPY SKGTKNLHTL YWEMLFSQQN LQNIVYKLNG | |
| | NAEIFYRKAS INQKDVVVHK ADLPIKNKDP QNSKKESMFD YDIIKDKRFT | |
| | CDKYQFHVPI TMNFKALGEN HFNRKVNRLI HDAENMHIIG IDRGERNLIY | |
| | LCMIDMKGNI VKQISLNEII SYDKNKLEHK RNYHQLLKTR EDENKSARQS | |
| | WQTIHTIKEL KEGYLSQVIH VITDLMVEYN AIVVLEDLNF GFKQGRQKFE | |
| | RQVYQKFEKM LIDKLNYLVD KSKGMDEDGG LLHAYQLTDE FKSFKQLGKQ | |
| | SGFLYYIPAW NTSKLDPTTG FVNLFYTKYE SVEKSKEFIN NFTSILYNQE | |
| | REYFEFLFDY SAFTSKAEGS RLKWTVCSKG ERVETYRNPK KNNEWDTQKI | |
| | DLTFELKKLF NDYSISLLDG DLREQMGKID KADFYKKFMK LFALIVQMRN | |
| | SDEREDKLIS PVLNKYGAFF ETGKNERMPL DADANGAYNI ARKGLWIIEK | |
| | IKNTDVEQLD KVKLTISNKE WLQYAQEHIL | |
| WP_035635841. | MSKLEKFTNC YSLSKTLRFK AIPVGKTQEN IDNKRLLVED EKRAEDYKGV | (SEQ |
| 1 | KKLLDRYYLS FINDVLHSIK LKNLNNYISL FRKKTRTEKE NKELENLEIN | ID NO: |
| type V CRISPR- | LRKEIAKAFK GNEGYKSLFK KDIIETILPE FLDDKDEIAL VNSFNGFTTA | 159) |
| associated | FTGFFDNREN MFSEEAKSTS IAFRCINENL TRYISNMDIF EKVDAIFDKH | |
| protein Cpf1 | EVQEIKEKIL NSDYDVEDFF EGEFFNFVLT QEGIDVYNAI IGGFVTESGE | |
| [Lachnospiraceae | KIKGLNEYIN LYNQKTKQKL PKFKPLYKQV LSDRESLSFY GEGYTSDEEV | |
| bacterium | LEVFRNTLNK NSEIFSSIKK LEKLFKNFDE YSSAGIFVKN GPAISTISKD | |
| ND2006] | IFGEWNVIRD KWNAEYDDIH LKKKAVVTEK YEDDRRKSFK KIGSFSLEQL | |
| | QEYADADLSV VEKLKEIIIQ KVDEIYKVYG SSEKLFDADF VLEKSLKKND | |
| | AVVAIMKDLL DSVKSFENYI KAFFGEGKET NRDESFYGDF VLAYDILLKV | |
| | DHIYDAIRNY VTQKPYSKDK FKLYFQNPQF MGGWDKDKET DYRATILRYG | |
| | SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS | |
| | NAYDFNFSET EKYKDIAGFY REVEEQGYKV SFESASKKEV DKLVEEGKLY | |
| | MFQIYNKDFS DKSHGTPNLH TMYFKLLFDE NNHGQIRLSG GAELFMRRAS | |
| | LKKEELVVHP ANSPIANKNP DNPKKTTTLS YDVYKDKRFS EDQYELHIPI | |
| | AINKCPKNIF KINTEVRVLL KHDDNPYVIG IDRGERNLLY IVVVDGKGNI | |
| | VEQYSLNEII NNFNGIRIKT DYHSLLDKKE KERFEARQNW TSIENIKELK | |
| | AGYISQWVHK ICELVEKYDA VIALEDLNSG FKNSRVKVEK QVYQKFEKML | |
| | IDKLNYMVDK KSNPCATGGA LKGYQITNKF ESFKSMSTQN GFIFYIPAWL | |
| | TSKIDPSTGF VNLLKTKYTS IADSKKFISS FDRIMYVPEE DLFEFALDYK | |
| | NFSRTDADYI KKWKLYSYGN RIRIFRNPKK NNVFDWEEVC LTSAYKELFN | |
| | KYGINYQQGD IRALLCEQSD KAFYSSFMAL MSLMLQMRNS ITGRTDVDFL | |
| | ISPVKNSDGI FYDSRNYEAQ ENAILPKNAD ANGAYNIARK VLWAIGQFKK | |
| | AEDEKLDKVK IAISNKEWLE YAQTSVKH | |
| WP_051666128. | MLKNVGIDRL DVEKGRKNMS KLEKFTNCYS LSKTLRFKAI PVGKTQENID | (SEQ |
| 1 | NKRLLVEDEK RAEDYKGVKK LLDRYYLSFI NDVLHSIKLK NLNNYISLFR | ID NO: |
| type V CRISPR- | KKTRTEKENK ELENLEINLR KEIAKAFKGN EGYKSLFKKD IIETILPEFL | 160) |
| associated | DDKDEIALVN SFNGFTTAFT GFFDNRENMF SEEAKSTSIA FRCINENLTR | |
| protein Cpf1 | YISNMDIFEK VDAIFDKHEV QEIKEKILNS DYDVEDFFEG EFFNFVLTQE | |
| [Lachnospiraceae | GIDVYNAIIG GFVTESGEKI KGLNEYINLY NQKTKQKLPK FKPLYKQVLS | |
| bacterium | DRESLSFYGE GYTSDEEVLE VFRNTLNKNS EIFSSIKKLE KLFKNFDEYS | |
| ND2006] | SAGIFVKNGP AISTISKDIF GEWNVIRDKW NAEYDDIHLK KKAVVTEKYE | |
| | DDRRKSFKKI GSFSLEQLQE YADADLSVVE KLKEIIIQKV DEIYKVYGSS | |
| | EKLFDADFVL EKSLKKNDAV VAIMKDLLDS VKSFENYIKA FFGEGKETNR | |
| | DESFYGDFVL AYDILLKVDH IYDAIRNYVT QKPYSKDKFK LYFQNPQFMG | |
| | GWDKDKETDY RATILRYGSK YYLAIMDKKY AKCLQKIDKD DVNGNYEKIN | |
| | YKLLPGPNKM LPKVFFSKKW MAYYNPSEDI QKIYKNGTFK KGDMFNLNDC | |
| | HKLIDFFKDS ISRYPKWSNA YDFNFSETEK YKDIAGFYRE VEEQGYKVSF | |
| | ESASKKEVDK LVEEGKLYMF QIYNKDFSDK SHGTPNLHTM YFKLLFDENN | |
| | HGQIRLSGGA ELFMRRASLK KEELVVHPAN SPIANKNPDN PKKTTTLSYD | |
| | VYKDKRFSED QYELHIPIAI NKCPKNIFKI NTEVRVLLKH DDNPYVIGID | |
| | RGERNLLYIV VVDGKGNIVE QYSLNEIINN FNGIRIKTDY HSLLDKKEKE | |
| | RFEARQNWTS IENIKELKAG YISQVVHKIC ELVEKYDAVI ALEDLNSGFK | |
| | NSRVKVEKQV YQKFEKMLID KLNYMVDKKS NPCATGGALK GYQITNKFES | |
| | FKSMSTQNGF IFYIPAWLTS KIDPSTGFVN LLKTKYTSIA DSKKFISSFD | |
| | RIMYVPEEDL FEFALDYKNF SRTDADYIKK WKLYSYGNRI RIFRNPKKNN | |
| | VFDWEEVCLT SAYKELFNKY GINYQQGDIR ALLCEQSDKA FYSSFMALMS | |
| | LMLQMRNSIT GRTDVDFLIS PVKNSDGIFY DSRNYEAQEN AILPKNADAN | |
| | GAYNIARKVL WAIGQFKKAE DEKLDKVKIA ISNKEWLEYA QTSVKH | |
| WP_015504779. | MDAKEFTGQY PLSKTLRFEL RPIGRTWDNL EASGYLAEDR HRAECYPRAK | (SEQ |
| 1 | ELLDDNHRAF LNRVLPQIDM DWHPIAEAFC KVHKNPGNKE LAQDYNLQLS | ID NO: |
| type V CRISPR- | KRRKEISAYL QDADGYKGLF AKPALDEAMK IAKENGNESD IEVLEAFNGF | 161) |
| associated | SVYFTGYHES RENIYSDEDM VSVAYRITED NFPRFVSNAL IFDKLNESHP | |
| protein Cpf1 | DIISEVSGNL GVDDIGKYFD VSNYNNFLSQ AGIDDYNHII GGHTTEDGLI | |
| [Candidatus | QAFNVVLNLR HQKDPGFEKI QFKQLYKQIL SVRTSKSYIP KQFDNSKEMV | |
| Methanomethylophilus | DCICDYVSKI EKSETVERAL KLVRNISSFD LRGIFVNKKN LRILSNKLIG | |
| alvus] | DWDAIETALM HSSSSENDKK SVYDSAEAFT LDDIFSSVKK FSDASAEDIG | |
| | NRAEDICRVI SETAPFINDL RAVDLDSLND DGYEAAVSKI RESLEPYMDL | |
| | FHELEIFSVG DEFPKCAAFY SELEEVSEQL IEIIPLFNKA RSFCTRKRYS | |
| | TDKIKVNLKF PTLADGWDLN KERDNKAAIL RKDGKYYLAI LDMKKDLSSI | |
| | RTSDEDESSF EKMEYKLLPS PVKMLPKIFV KSKAAKEKYG LTDRMLECYD | |
| | KGMHKSGSAF DLGFCHELID YYKRCIAEYP GWDVFDFKFR ETSDYGSMKE | |
| | FNEDVAGAGY YMSLRKIPCS EVYRLLDEKS IYLFQIYNKD YSENAHGNKN | |
| | MHTMYWEGLF SPQNLESPVF KLSGGAELFF RKSSIPNDAK TVHPKGSVLV | |
| | PRNDVNGRRI PDSIYRELTR YFNRGDCRIS DEAKSYLDKV KTKKADHDIV | |
| | KDRRFTVDKM MFHVPIAMNF KAISKPNLNK KVIDGIIDDQ DLKIIGIDRG | |
| | ERNLIYVTMV DRKGNILYQD SLNILNGYDY RKALDVREYD NKEARRNWTK | |
| | VEGIRKMKEG YLSLAVSKLA DMIIENNAII VMEDLNHGFK AGRSKIEKQV | |
| | YQKFESMLIN KLGYMVLKDK SIDQSGGALH GYQLANHVTT LASVGKQCGV | |
| | IFYIPAAFTS KIDPTTGFAD LFALSNVKNV ASMREFFSKM KSVIYDKAEG | |
| | KFAFTFDYLD YNVKSECGRT LWTVYTVGER FTYSRVNREY VRKVPTDIIY | |
| | DALQKAGISV EGDLRDRIAE SDGDTLKSIF YAFKYALDMR VENREEDYIQ | |
| | SPVKNASGEF FCSKNAGKSL PQDSDANGAY NIALKGILQL RMLSEQYDPN | |
| | AESIRLPLIT NKAWLTFMQS GMKTWKN | |
| WP_044910713. | MGLYDGFVNR YSVSKTLRFE LIPQGRTREY IETNGILSDD EERAKDYKTI | (SEQ |
| 1 | KRLIDEYHKD YISRCLKNVN ISCLEEYYHL YNSSNRDKRH EELDALSDQM | ID NO: |
| type V CRISPR- | RGEIASFLTG NDEYKEQKSR DIIINERIIN FASTDEELAA VKRFRKFTSY | 162) |
| associated | FTGFFTNREN MYSAEKKSTA IAHRIIDVNL PKYVDNIKAF NTAIEAGVFD | (SEQ |
| protein Cpf1 | IAEFESNFKA ITDEHEVSDL LDITKYSRFI RNEDIIIYNT LLGGISMKDE | |
| [Lachnospiraceae | KIQGLNELIN LHNQKHPGKK VPLLKVLYKQ ILGDSQTHSF VDDQFEDDQQ | |
| bacterium | VINAVKAVTD TFSETLLGSL KIIINNIGHY DLDRIYIKAG QDITTLSKRA | |
| MC2017] | LNDWHIITEC LESEYDDKFP KNKKSDTYEE MRNRYVKSFK SFSIGRLNSL | |
| | VTTYTEQACF LENYLGSFGG DTDKNCLTDF TNSLMEVEHL LNSEYPVTNR | |
| | LITDYESVRI LKRLLDSEME VIHFLKPLLG NGNESDKDLV FYGEFEAEYE | |
| | KLLPVIKVYN RVRNYLTRKP FSTEKIKLNF NSPTLLCGWS QSKEKEYMGV | |
| | ILRKDGQYYL GIMTPSNKKI FSEAPKPDED CYEKMVLRYI PHPYQMLPKV | |
| | FFSKSNIAFF NPSDEILRIK KQESFKKGKS FNRDDCHKFI DFYKDSINRH | |
| | EEWRKFNFKF SDTDSYEDIS RFYKEVENQA FSMSFTKIPT VYIDSLVDEG | |
| | KLYLFKLHNK DFSEHSKGKP NLHTVYWNAL FSEYNLQNTV YQLNGSAEIF | |
| | FRKASIPENE RVIHKKNVPI TRKVAELNGK KEVSVFPYDI IKNRRYTVDK | |
| | FQFHVPLKMN FKADEKKRIN DDVIEAIRSN KGIHVIGIDR GERNLLYLSL | |
| | INEEGRIIEQ RSLNIIDSGE GHTQNYRDLL DSREKDREKA RENWQEIQEI | |
| | KDLKTGYLSQ AIHTITKWMK EYNAIIVLED LNDRFTNGRK KVEKQVYQKF | |
| | EKMLIDKLNY YVDKDEEFDR MGGTHRALQL TEKFESFQKL GRQTGFIFYV | |
| | PAWNTSKLDP TTGFVDLLYP KYKSVDATKD FIKKFDFIRF NSEKNYFEFG | |
| | LHYSNFTERA IGCRDEWILC SYGNRIVNFR NAAKNNSWDY KEIDITKQLL | |
| | DLFEKNGIDV KQENLIDSIC EMKDKPFFKS LIANIKLILQ IRNSASGTDI | |
| | DYMISPAMND RGEFFDTRKG LQQLPLDADA NGAYNIAKKG LWIVDQIRNT | |
| | TGNNVKMAMS NREWMHFAQE SRLA | |
| KKQ36153.1 | MKNVFGGFTN LYSLTKTLRF ELKPTSKTQK LMKRNNVIQT DEEIDKLYHD | ID NO: |
| hypothetical | EMKPILDEIH RRFINDALAQ KIFISASLDN FLKVVKNYKV ESAKKNIKQN | 163) |
| protein | QVKLLQKEIT IKTLGLRREV VSGFITVSKK WKDKYVGLGI KLKGDGYKVL | |
| US52_C0007G0 | TEQAVLDILK IEFPNKAKYI DKFRGFWTYF SGFNENRKNY YSEEDKATSI | |
| 008 [candidate | ANRIVNENLS RYIDNIIAFE EILQKIPNLK KFKQDLDITS YNYYLNQAGI | |
| division WS6 | DKYNKIIGGY IVDKDKKIQG INEKVNLYTQ QTKKKLPKLK FLFKQIGSER | |
| bacterium | KGFGIFEIKE GKEWEQLGDL FKLQRTKINS NGREKGLFDS LRTMYREFFD | |
| GW2011_GWA2 | EIKRDSNSQA RYSLDKIYFN KASVNTISNS WFTNWNKFAE LLNIKEDKKN | |
| 37_6] | GEKKIPEQIS IEDIKDSLSI IPKENLEELF KLTNREKHDR TRFFGSNAWV | |
| | TFLNIWQNEI EESFNKLEEK EKDFKKNAAI KFQKNNLVQK NYIKEVCDRM | |
| | LAIERMAKYH LPKDSNLSRE EDFYWIIDNL SEQREIYKYY NAFRNYISKK | |
| | PYNKSKMKLN FENGNLLGGW SDGQERNKAG VILRNGNKYY LGVLINRGIF | |
| | RTDKINNEIY RTGSSKWERL ILSNLKFQTL AGKGFLGKHG VSYGNMNPEK | |
| | SVPSLQKFIR ENYLKKYPQL TEVSNTKFLS KKDFDAAIKE ALKECFTMNF | |
| | INIAENKLLE AEDKGDLYLF EITNKDFSGK KSGKDNIHTI YWKYLFSESN | |
| | CKSPIIGLNG GAEIFFREGQ KDKLHTKLDK KGKKVFDAKR YSEDKLFFHV | |
| | SITINYGKPK NIKFRDIINQ LITSMNVNII GIDRGEKHLL YYSVIDSNGI | |
| | ILKQGSLNKI RVGDKEVDFN KKLTERANEM KKARQSWEQI GNIKNFKEGY | |
| | LSQAIHEIYQ LMIKYNAIIV LEDLNTEFKA KRLSKVEKSV YKKFELKLAR | |
| | KLNHLILKDR NTNEIGGVLK AYQLTPTIGG GDVSKFEKAK QWGMMFYVRA | |
| | NYTSTTDPVT GWRKHLYISN FSNNSVIKSF FDPTNRDTGI EIFYSGKYRS | |
| | WGFRYVQKET GKKWELFATK ELERFKYNQT TKLCEKINLY DKFEELFKGI | |
| | DKSADIYSQL CNVLDFRWKS LVYLWNLLNQ IRNVDKNAEG NKNDFIQSPV | |
| | YPFFDSRKTD GKTEPINGDA NGALNIARKG LMLVERIKNN PEKYEQLIRD | |
| | TEWDAWIQNF NKVN | |
| WP_044919442. | MYYESLTKQY PVSKTIRNEL IPIGKTLDNI RQNNILESDV KRKQNYEHVK | (SEQ |
| 1 | GILDEYHKQL INEALDNCTL PSLKIAAEIY LKNQKEVSDR EDFNKTQDLL | ID NO: |
| type V CRISPR- | RKEWVEKLKA HENFTKIGKK DILDLLEKLP SISEDDYNAL ESFRNFYTYF | 164) |
| associated | TSYNKVRENL YSDKEKSSTV AYRLINENFP KFLDNVKSYR FVKTAGILAD | |
| protein Cpf1 | GLGEEEQDSL FIVETFNKTL TQDGIDTYNS QVGKINSSIN LYNQKNQKAN | |
| [Lachnospiraceae | GFRKIPKMKM LYKQILSDRE ESFIDEFQSD EVLIDNVESY GSVLIESLKS | |
| bacterium | SKVSAFFDAL RESKGKNVYV KNDLAKTAMS NIVFENWRTF DDLLNQEYDL | |
| MA2020] | ANENKKKDDK YFEKRQKELK KNKSYSLEHL CNLSEDSCNL IENYIHQISD | |
| | DIENIIINNE TFLRIVINEH DRSRKLAKNR KAVKAIKDFL DSIKVLEREL | |
| | KLINSSGQEL EKDLIVYSAH EELLVELKQV DSLYNMTRNY LTKKPFSTEK | |
| | VKLNFNRSTL LNGWDRNKET DNLGVLLLKD GKYYLGIMNT SANKAFVNPP | |
| | VAKTEKVFKK VDYKLLPVPN QMLPKVFFAK SNIDFYNPSS EIYSNYKKGT | |
| | HKKGNMFSLE DCHNLIDFFK ESISKHEDWS KFGFKFSDTA SYNDISEFYR | |
| | EVEKQGYKLT YTDIDETYIN DLIERNELYL FQIYNKDFSM YSKGKLNLHT | |
| | LYFMMLFDQR NIDDVVYKLN GEAEVFYRPA SISEDELIIH KAGEEIKNKN | |
| | PNRARTKETS TFSYDIVKDK RYSKDKFTLH IPITMNFGVD EVKRFNDAVN | |
| | SAIRIDENVN VIGIDRGERN LLYVVVIDSK GNILEQISLN SIINKEYDIE | |
| | TDYHALLDER EGGRDKARKD WNTVENIRDL KAGYLSQVVN VVAKLVLKYN | |
| | AIICLEDLNF GFKRGRQKVE KQVYQKFEKM LIDKLNYLVI DKSREQTSPK | |
| | ELGGALNALQ LTSKFKSFKE LGKQSGVIYY VPAYLTSKID PTTGFANLFY | |
| | MKCENVEKSK RFFDGFDFIR FNALENVFEF GFDYRSFTQR ACGINSKWTV | |
| | CTNGERIIKY RNPDKNNMFD EKVVVVTDEM KNLFEQYKIP YEDGRNVKDM | |
| | IISNEEAEFY RRLYRLLQQT LQMRNSTSDG TRDYIISPVK NKREAYFNSE | |
| | LSDGSVPKDA DANGAYNIAR KGLWVLEQIR QKSEGEKINL AMTNAEWLEY | |
| | AQTHLL | |
| WP_035798880. | MYYQNLTKKY PVSKTIRNEL IPIGKTLENI RKNNILESDV KRKQDYEHVK | (SEQ |
| 1 | GIMDEYHKQL INEALDNYML PSLNQAAEIY LKKHVDVEDR EEFKKTQDLL | ID NO: |
| type V CRISPR- | RREVTGRLKE HENYTKIGKK DILDLLEKLP SISEEDYNAL ESFRNFYTYF | 165) |
| associated | TSYNKVRENL YSDEEKSSTV AYRLINENLP KFLDNIKSYA FVKAAGVLAD | |
| protein Cpf1 | CIEEEEQDAL FMVETFNMTL TQEGIDMYNY QIGKVNSAIN LYNQKNHKVE | |
| [Butyrivibrio sp. | EFKKIPKMKV LYKQILSDRE EVFIGEFKDD ETLLSSIGAY GNVLMTYLKS | |
| NC3005] | EKINIFFDAL RESEGKNVYV KNDLSKTTMS NIVFGSWSAF DELLNQEYDL | |
| | ANENKKKDDK YFEKRQKELK KNKSYTLEQM SNLSKEDISP IENYIERISE | |
| | DIEKICIYNG EFEKIVVNEH DSSRKLSKNI KAVKVIKDYL DSIKELEHDI | |
| | KLINGSGQEL EKNLVVYVGQ EEALEQLRPV DSLYNLTRNY LTKKPFSTEK | |
| | VKLNFNKSTL LNGWDKNKET DNLGILFFKD GKYYLGIMNT TANKAFVNPP | |
| | AAKTENVFKK VDYKLLPGSN KMLPKVFFAK SNIGYYNPST ELYSNYKKGT | |
| | HKKGPSFSID DCHNLIDFFK ESIKKHEDWS KFGFEFSDTA DYRDISEFYR | |
| | EVEKQGYKLT FTDIDESYIN DLIEKNELYL FQIYNKDFSE YSKGKLNLHT | |
| | LYFMMLFDQR NLDNVVYKLN GEAEVFYRPA SIAENELVIH KAGEGIKNKN | |
| | PNRAKVKETS TFSYDIVKDK RYSKYKFTLH IPITMNFGVD EVRRENDVIN | |
| | NALRTDDNVN VIGIDRGERN LLYVVVINSE GKILEQISLN SIINKEYDIE | |
| | TNYHALLDER EDDRNKARKD WNTIENIKEL KTGYLSQVVN VVAKLVLKYN | |
| | AIICLEDLNF GFKRGRQKVE KQVYQKFEKM LIEKLNYLVI DKSREQVSPE | |
| | KMGGALNALQ LTSKFKSFAE LGKQSGIIYY VPAYLTSKID PTTGFVNLFY | |
| | IKYENIEKAK QFFDGFDFIR FNKKDDMFEF SFDYKSFTQK ACGIRSKWIV | |
| | YTNGERIIKY PNPEKNNLFD EKVINVTDEI KGLFKQYRIP YENGEDIKEI | |
| | IISKAEADFY KRLFRLLHQT LQMRNSTSDG TRDYIISPVK NDRGEFFCSE | |
| | FSEGTMPKDA DANGAYNIAR KGLWVLEQIR QKDEGEKVNL SMTNAEWLKY | |
| | AQLHLL | |
| WP_027109509. | MENYYDSLTR QYPVTKTIRQ ELKPVGKTLE NIKNAEIIEA DKQKKEAYVK | (SEQ |
| 1 | VKELMDEFHK SIIEKSLVGI KLDGLSEFEK LYKIKTKTDE DKNRISELFY | ID NO: |
| type V CRISPR- | YMRKQIADAL KNSRDYGYVD NKDLIEKILP ERVKDENSLN ALSCFKGFTT | 166) |
| associated | YFTDYYKNRK NIYSDEEKHS TVGYRCINEN LLIFMSNIEV YQIYKKANIK | |
| protein Cpf1 | NDNYDEETLD KTFMIESFNE CLTQSGVEAY NSVVASIKTA TNLYIQKNNK | |
| [Lachnospiraceae | EENFVRVPKM KVLFKQILSD RTSLFDGLII ESDDELLDKL CSFSAEVDKF | |
| bacterium | LPINIDRYIK TLMDSNNGTG IYVKNDSSLT TLSNYLTDSW SSIRNAFNEN | |
| NC2008] | YDAKYTGKVN DKYEEKREKA YKSNDSFELN YIQNLLGINV IDKYIERINF | |
| | DIKEICEAYK EMTKNCFEDH DKTKKLQKNI KAVASIKSYL DSLKNIERDI | |
| | KLINGTGLES RNEFFYGEQS TVLEEITKVD ELYNITRNYL TKKPFSTEKM | |
| | KLNFNNPQLL GGWDVNKERD CYGVILIKDN NYYLGIMDKS ANKSFLNIKE | |
| | SKNENAYKKV NCKLLPGPNK MFPKVFFAKS NIDYYDPTHE IKKLYDKGTF | |
| | KKGNSFNLED CHKLIDFYKE SIKKNDDWKN FNFNFSDTKD YEDISGFFRE | |
| | VEAQNYKITY TNVSCDFIES LVDEGKLYLF QIYNKDFSEY ATGNLNLHTL | |
| | YLKMLFDERN LKDLCIKMNG EAEVFYRPAS ILDEDKVVHK ANQKITNKNT | |
| | NSKKKESIFS YDIVKDKRYT VDKFFIHLPI TLNYKEQNVS RFNDYIREIL | |
| | KKSKNIRVIG IDRGERNLLY VVVCDSDGSI LYQRSINEIV SGSHKTDYHK | |
| | LLDNKEKERL SSRRDWKTIE NIKDLKAGYM SQVVNEIYNL ILKYNAIVVL | |
| | EDLNIGFKNG RKKVEKQVYQ NFEKALIDKL NYLCIDKTRE QLSPSSPGGV | |
| | LNAYQLTAKF ESFEKIGKQT GCIFYVPAYL TSQIDPTTGF VNLFYQKDTS | |
| | KQGLQLFFRK FKKINFDKVA SNFEFVFDYN DFTNKAEGTK TNWTISTQGT | |
| | RIAKYRSDDA NGKWISRTVH PTDIIKEALN REKINYNDGH DLIDEIVSIE | |
| | KSAVLKEIYY GFKLTLQLRN STLANEEEQE DYIISPVKNS SGNYFDSRIT | |
| | SKELPCDADA NGAYNIARKG LWALEQIRNS ENVSKVKLAI SNKEWFEYTQ | |
| | NNIPSL | |
| WP_049895985. | METEILKYDF FEREGKYMYY DGLTKQYALS KTIRNELVPI GKTLDNIKKN | (SEQ |
| 1 | RILEADIKRK SDYEHVKKLM DMYHKKIINE ALDNFKLSVL EDAADIYFNK | ID NO: |
| type V CRISPR- | QNDERDIDAF LKIQDKLRKE IVEQLKGHTD YSKVGNKDFL GLLKAASTEE | 167) |
| associated | DRILIESFDN FYTYFTSYNK VRSNLYSAED KSSTVAYRLI NENLPKFFDN | |
| protein Cpf1 | IKAYRTVRNA GVISGDMSIV EQDELFEVDT FNHTLTQYGI DTYNHMIGQL | |
| [Oribacterium sp. | NSAINLYNQK MHGAGSFKKL PKMKELYKQL LTEREEEFIE EYTDDEVLIT | |
| NK2B42] | SVHNYVSYLI DYLNSDKVES FFDTLRKSDG KEVFIKNDVS KTTMSNILFD | |
| WP_029202018 | NWSTIDDLIN HEYDSAPENV KKTKDDKYFE KRQKDLKKNK SYSLSKIAAL | |
| | CRDTTILEKY IRRLVDDIEK IYTSNNVFSD IVLSKHDRSK KLSKNTNAVQ | |
| | AIKNMLDSIK DFEHDVMLIN GSGQEIKKNL NVYSEQEALA GILRQVDHIY | |
| | NLTRNYLTKK PFSTEKIKLN FNRPTFLDGW DKNKEEANLG ILLIKDNRYY | |
| | LGIMNTSSNK AFVNPPKAIS NDIYKKVDYK LLPGPNKMLP KVFFATKNIA | |
| | YYAPSEELLS KYRKGTHKKG DSFSIDDCRN LIDFFKSSIN KNTDWSTFGF | |
| | NFSDTNSYND ISDFYREVEK QGYKLSFTDI DACYIKDLVD NNELYLFQIY | |
| | NKDFSPYSKG KLNLHTLYFK MLFDQRNLDN VVYKLNGEAE VFYRPASIES | |
| | DEQIIHKSGQ NIKNKNQKRS NCKKTSTEDY DIVKDRRYCK DKFMLHLPIT | |
| | VNFGTNESGK FNELVNNAIR ADKDVNVIGI DRGERNLLYV VVVDPCGKII | |
| | EQISLNTIVD KEYDIETDYH QLLDEKEGSR DKARKDWNTI ENIKELKEGY | |
| | LSQWVNIIAK LVLKYDAIIC LEDLNFGFKR GRQKVEKQVY QKFEKMLIDK | |
| | MNYLVLDKSR KQESPQKPGG ALNALQLTSA FKSFKELGKQ TGIIYYVPAY | |
| | LTSKIDPTTG FANLFYIKYE SVDKARDFFS KFDFIRYNQM DNYFEFGFDY | |
| | KSFTERASGC KSKWIACTNG ERIVKYRNSD KNNSFDDKTV ILTDEYRSLF | |
| | DKYLQNYIDE DDLKDQILQI DSADFYKNLI KLFQLTLQMR NSSSDGKRDY | |
| | IISPVKNYRE EFFCSEFSDD TFPRDADANG AYNIARKGLW VIKQIRETKS | |
| | GTKINLAMSN SEWLEYAQCN LL | |
| WP_028248456. | MYYQNLTKMY PISKTLRNEL IPVGKTLENI RKNGILEADI QRKADYEHVK | (SEQ |
| 1 | KLMDNYHKQL INEALQGVHL SDLSDAYDLY FNLSKEKNSV DAFSKCQDKL | ID NO: |
| type V CRISPR- | RKEIVSLLKN HENFPKIGNK EIIKLLQSLY DNDTDYKALD SFSNFYTYFS | 168) |
| associated | SYNEVRKNLY SDEEKSSTVA YRLINENLPK FLDNIKAYAI AKKAGVRAEG | |
| protein Cpf1 | LSEEDQDCLF IIETFERTLT QDGIDNYNAA IGKLNTAINL FNQQNKKQEG | |
| [Pseudobutyrivibrio | FRKVPQMKCL YKQILSDREE AFIDEFSDDE DLITNIESFA ENMNVFLNSE | |
| ruminis] | IITDFKIALV ESDGSLVYIK NDVSKTSFSN IVFGSWNAID EKLSDEYDLA | |
| | NSKKKKDEKY YEKRQKELKK NKSYDLETII GLFDDNSDVI GKYIEKLESD | |
| | ITAIAEAKND FDEIVLRKHD KNKSLRKNTN AVEAIKSYLD TVKDFERDIK | |
| | LINGSGQEVE KNLVVYAEQE NILAEIKNVD SLYNMSRNYL TQKPFSTEKF | |
| | KLNFNRATLL NGWDKNKETD NLGILFEKDG MYYLGIMNTK ANKIFVNIPK | |
| | ATSNDVYHKV NYKLLPGPNK MLPKVFFAQS NLDYYKPSEE LLAKYKAGTH | |
| | KKGDNFSLED CHALIDFFKA SIEKHPDWSS FGFEFSETCT YEDLSGFYRE | |
| | VEKQGYKITY TDVDADYITS LVERDELYLF QIYNKDFSPY SKGNLNLHTI | |
| | YLQMLFDQRN LNNVVYKLNG EAEVFYRPAS INDEEVIIHK AGEEIKNKNS | |
| | KRAVDKPTSK FGYDIIKDRR YSKDKFMLHI PVTMNFGVDE TRRFNDVVND | |
| | ALRNDEKVRV IGIDRGERNL LYVWVVDTDG TILEQISLNS IINNEYSIET | |
| | DYHKLLDEKE GDRDRARKNW TTIENIKELK EGYLSQVVNV IAKLVLKYNA | |
| | IICLEDLNFG FKRGRQKVEK QVYQKFEKML IDKLNYLVID KSRKQDKPEE | |
| | FGGALNALQL TSKFTSFKDM GKQTGIIYYV PAYLTSKIDP TTGFANLFYV | |
| | KYENVEKAKE FFSRFDSISY NNESGYFEFA FDYKKFTDRA CGARSQWTVC | |
| | TYGERIIKFR NTEKNNSFDD KTIVLSEEFK ELFSIYGISY EDGAELKNKI | |
| | MSVDEADFFR SLTRLFQQTM QMRNSSNDVT RDYIISPIMN DRGEFFNSEA | |
| | CDASKPKDAD ANGAFNIARK GLWVLEQIRN TPSGDKLNLA MSNAEWLEYA | |
| | QRNQI | |
| WP_028830240 | MENFKNLYPI NKTLRFELRP YGKTLENFKK SGLLEKDAFK ANSRRSMQAI | (SEQ |
| type V CRISPR- | IDEKFKETIE ERLKYTEFSE CDLGNMTSKD KKITDKAATN LKKQVILSFD | ID NO: |
| associated | DEIFNNYLKP DKNIDALFKN DPSNPVISTF KGFTTYFVNF FEIRKHIFKG | 169) |
| protein Cpf1 | ESSGSMAYRI IDENLTTYLN NIEKIKKLPE ELKSQLEGID QIDKLNNYNE | |
| [Proteocatella | FITQSGITHY NEIIGGISKS ENVKIQGINE GINLYCQKNK VKLPRLTPLY | |
| sphenisci] | KMILSDRVSN SFVLDTIEND TELIEMISDL INKTEISQDV IMSDIQNIFI | |
| | KYKQLGNLPG ISYSSIVNAI CSDYDNNFGD GKRKKSYEND RKKHLETNVY | |
| | SINYISELLT DTDVSSNIKM RYKELEQNYQ VCKENFNATN WMNIKNIKQS | |
| | EKTNLIKDLL DILKSIQRFY DLFDIVDEDK NPSAEFYTWL SKNAEKLDFE | |
| | FNSVYNKSRN YLTRKQYSDK KIKLNFDSPT LAKGWDANKE IDNSTIIMRK | |
| | FNNDRGDYDY FLGIWNKSTP ANEKIIPLED NGLFEKMQYK LYPDPSKMLP | |
| | KQFLSKIWKA KHPTTPEFDK KYKEGRHKKG PDFEKEFLHE LIDCFKHGLV | |
| | NHDEKYQDVF GFNLRNTEDY NSYTEFLEDV ERCNYNLSFN KIADTSNLIN | |
| | DGKLYVFQIW SKDFSIDSKG TKNLNTIYFE SLFSEENMIE KMFKLSGEAE | |
| | IFYRPASLNY CEDIIKKGHH HAELKDKFDY PIIKDKRYSQ DKFFFHVPMV | |
| | INYKSEKLNS KSLNNRTNEN LGQFTHIIGI DRGERHLIYL TVVDVSTGEI | |
| | VEQKHLDEII NTDTKGVEHK THYLNKLEEK SKTRDNERKS WEAIETIKEL | |
| | KEGYISHVIN EIQKLQEKYN ALIVMENLNY GFKNSRIKVE KQVYQKFETA | |
| | LIKKENYIID KKDPETYIHG YQLTNPITTL DKIGNQSGIV LYIPAWNTSK | |
| | IDPVTGFVNL LYADDLKYKN QEQAKSFIQK IDNIYFENGE FKFDIDFSKW | |
| | NNRYSISKTK WTLTSYGTRI QTFRNPQKNN KWDSAEYDLT EEFKLILNID | |
| | GTLKSQDVET YKKFMSLFKL MLQLRNSVTG TDIDYMISPV TDKTGTHFDS | |
| | RENIKNLPAD ADANGAYNIA RKGIMAIENI MNGISDPLKI SNEDYLKYIQ | |
| | NQQE | |
| WP_084502895. | MIILYISTSN MNMEGVFMEN FKNLYPINKT LRFELRPYGK TLENFKKSGL | (SEQ |
| 1 | LEKDAFKANS RRSMQAIIDE KFKETIEERL KYTEFSECDL GNMTSKDKKI | ID NO: |
| type V CRISPR- | TDKAATNLKK QVILSFDDEI FNNYLKPDKN IDALFKNDPS NPVISTFKGF | 170) |
| associated | TTYFVNFFEI RKHIFKGESS GSMAYRIIDE NLTTYLNNIE KIKKLPEELK | |
| protein Cpf1 | SQLEGIDQID KLNNYNEFIT QSGITHYNEI IGGISKSENV KIQGINEGIN | |
| [Proteocatella | LYCQKNKVKL PRLTPLYKMI LSDRVSNSFV LDTIENDTEL IEMISDLINK | |
| sphenisci] | TEISQDVIMS DIQNIFIKYK QLGNLPGISY SSIVNAICSD YDNNFGDGKR | |
| | KKSYENDRKK HLETNVYSIN YISELLTDTD VSSNIKMRYK ELEQNYQVCK | |
| | ENFNATNWMN IKNIKQSEKT NLIKDLLDIL KSIQRFYDLF DIVDEDKNPS | |
| | AEFYTWLSKN AEKLDFEFNS VYNKSRNYLT RKQYSDKKIK LNFDSPTLAK | |
| | GWDANKEIDN STIIMRKFNN DRGDYDYFLG IWNKSTPANE KIIPLEDNGL | |
| | FEKMQYKLYP DPSKMLPKQF LSKIWKAKHP TTPEFDKKYK EGRHKKGPDF | |
| | EKEFLHELID CFKHGLVNHD EKYQDVFGFN LRNTEDYNSY TEFLEDVERC | |
| | NYNLSFNKIA DTSNLINDGK LYVFQIWSKD FSIDSKGTKN LNTIYFESLF | |
| | SEENMIEKMF KLSGEAEIFY RPASLNYCED IIKKGHHHAE LKDKFDYPII | |
| | KDKRYSQDKF FFHVPMVINY KSEKLNSKSL NNRTNENLGQ FTHIIGIDRG | |
| | ERHLIYLTVV DVSTGEIVEQ KHLDEIINTD TKGVEHKTHY LNKLEEKSKT | |
| | RDNERKSWEA IETIKELKEG YISHVINEIQ KLQEKYNALI VMENLNYGFK | |
| | NSRIKVEKQV YQKFETALIK KENYIIDKKD PETYIHGYQL TNPITTLDKI | |
| | GNQSGIVLYI PAWNTSKIDP VTGFVNLLYA DDLKYKNQEQ AKSFIQKIDN | |
| | IYFENGEFKF DIDFSKWNNR YSISKTKWTL TSYGTRIQTF RNPQKNNKWD | |
| | SAEYDLTEEF KLILNIDGTL KSQDVETYKK FMSLFKLMLQ LRNSVTGTDI | |
| | DYMISPVTDK TGTHFDSREN IKNLPADADA NGAYNIARKG IMAIENIMNG | |
| | ISDPLKISNE DYLKYIQNQQ E | |
| WP_055225123. | MNNGTNNFQN FIGISSLQKT LRNALIPTET TQQFIVKNGI IKEDELRGEN | (SEQ |
| 1 | RQILKDIMDD YYRGFISETL SSIDDIDWTS LFEKMEIQLK NGDNKDTLIK | ID NO: |
| Eubacterium | EQTEYRKAIH KKFANDDRFK NMFSAKLISD ILPEFVIHNN NYSASEKEEK | 171) |
| rectale | TQVIKLFSRF ATSFKDYFKN RANCFSADDI SSSSCHRIVN DNAEIFFSNA | |
| | LVYRRIVKSL SNDDINKISG DMKDSLKEMS LEEIYSYEKY GEFITQEGIS | |
| | FYNDICGKVN SFMNLYCQKN KENKNLYKLQ KLHKQILCIA DTSYEVPYKF | |
| | ESDEEVYQSV NGFLDNISSK HIVERLRKIG DNYNGYNLDK IYIVSKFYES | |
| | VSQKTYRDWE TINTALEIHY NNILPGNGKS KADKVKKAVK NDLQKSITEI | |
| | NELVSNYKLC SDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HIKSSKDFDI TFCHDLIDYF KNCIAIHPEW | |
| | KNFGFDFSDT STYEDISGFY REVELQGYKI DWTYISEKDI DLLQEKGQLY | |
| | LFQIYNKDFS KKSTGNDNLH TMYLKNLFSE ENLKDIVLKL NGEAEIFFRK | |
| | SSIKNPIIHK KGSILVNRTY EAEEKDQFGN IQIVRKNIPE NIYQELYKYF | |
| | NDKSDKELSD EAAKLKNVVG HHEAATNIVK DYRYTYDKYF LHMPITINFK | |
| | ANKTGFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPDKL KNVGHQCGCI FYVPAAYTSK IDPTTGFVNI | |
| | FKFKDLTVDA KREFIKKFDS IRYDSEKNLF CFTFDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFRLTV QMRNSLSELE DRDYDRLISP VLNENNIFYD | |
| | SAKAGDALPK DADANGAYCI ALKGLYEIKQ ITENWKEDGK FSRDKLKISN | |
| | KDWFDFIQNK RYL | |
| WP_055237260. | MNNGTNNFQN FIGISSLQKT LRNALIPTET TQQFIVKNGI IKEDELRGEN | (SEQ |
| 1 | RQILKDIMDD YYRGFISETL SSIDDIDWTS LFEKMEIQLK NGDNKDTLIK | ID NO: |
| Eubacterium | EQAEKRKAIY KKFADDDRFK NMFSAKLISD ILPEFVIHNN NYSASEKEEK | 172) |
| rectale | TQVIKLFSRF ATSFKDYFKN RANCFSADDI SSSSCHRIVN DNAEIFFSNA | |
| | LVYRRIVKNL SNDDINKISG DMKDSLKEMS LDEIYSYEKY GEFITQEGIS | |
| | FYNDICGKVN SFMNLYCQKN KENKNLYKLR KLHKQILCIA DTSYEVPYKF | |
| | ESDEEVYQSV NGFLDNISSK HIVERLRKIG DNYNGYNLDK IYIVSRFYES | |
| | VSQKTYRDWE TINTALEIHY NNILPGNGKS KADKVKKAVK NDLQKSITEI | |
| | NELVSNYKLC PDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HLKSSKDFDI TFCRDLIDYF KNCIAIHPEW | |
| | KNFGFDFSDT STYEDISGFY REVELQGYKI DWTYISEKDI DLLQEKGQLY | |
| | LFQIYNKDFS KKSTGNDNLH TMYLKNLFSE ENLKDIVLKL NGEAEIFFRK | |
| | SSIKNPIIHK KGSILVNRTY EAEEKDQFGN IQIVRKTIPE NIYQELYKYF | |
| | NDKSDKELSD EAAKLKNWVG HHEAATNIVK DYRYTYDKYF LHMPITINFK | |
| | ANKTSFINDR ILQYIAKEND LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPEKL KNVGHQCGCI FYVPAAYTSK IDPTTGFANI | |
| | FKFKDLTVDA KREFIKKFDS IRYDSEKNLF CFTFDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFKLTV QMRNSLSELE DRDYDRLISP VLNENNIFYD | |
| | SAKAGDALPK DADANGAYCI ALKGLYEIKQ ITENWKEDGK FSRDKLKISN | |
| | KDWFDFIQNK RYL | |
| WP_055272206. | MNNGTNNFQN FIGISSLQKT LRNALTPTET TQQFIVKNGI IKEDELRGEN | (SEQ |
| 1 | RQILKDIMDD YYRGFISETL SSIDDIDWTS LFEKMEIQLK NGDNKDTLIK | ID NO: |
| Eubacterium | EQAEKRKAIY KKFADDDRFK NMFSAKLISD ILPEFVIHNN NYSASEKEEK | 173) |
| rectale | TQVIKLFSRF ATSFKDYFKN RANCFSADDI SSSSCHRIVN DNAEIFFSNA | |
| | LVYRRIVKNL SNDDINKISG DMKDSLKKMS LEKIYSYEKY GEFITQEGIS | |
| | FYNDICGKVN SFMNLYCQKN KENKNLYKLR KLHKQILCIA DTSYEVPYKF | |
| | ESDEEVYQSV NGFLDNISSK HIVERLRKIG DNYNGYNLDK IYIVSKFYES | |
| | VSQKTYRDWE TINTALEIHY NNILPGNGKS KADKVKKAVK NDLQKSITEI | |
| | NELVSNYKLC PDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PEKKIIEGNT SENKGDYKKM IYNLLPGPNK MIPKVFLSSK | |
| | TGVETYKPSA YILEGYKQNK HLKSSKDFDI TFCRDLIDYF KNCIAIHPEW | |
| | KNFGFDFSDT STYEDISGFY REVELQGYKI DWTYISEKDI DLLQEKGQLY | |
| | LFQIYNKDFS KKSTGNDNLH TMYLKNLFSE ENLKDVVLKL NGEAEIFFRK | |
| | SSIKNPIIHK KGSILVNRTY EAEEKDQFGN IQIVRKTIPE NIYQELYKYF | |
| | NDKSDKELSD EAAKLKNAVG HHEAATNIVK DYRYTYDKYF LHMPITINFK | |
| | ANKTSFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPEKL KNVGHQCGCI FYVPAAYTSK IDPTTGFVNI | |
| | FKFKDLTVDA KREFIKKFDS IRYDSDKNLF CFTFDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFKLTV QMRNSLSELE DRNYDRLISP VLNENNIFYD | |
| | SAKAGDALPK DADANGAYCI ALKGLYEIKQ ITENWKEDGK FSRDKLKISN | |
| | KDWFDFIQNK RYL | |
| OLA16049.1 | MNNGTNNFQN FIGISSLQKT LRNALIPTET TQQFIVKNGI IKEDELRGKN | (SEQ |
| Eubacterium sp. | RQILKDIMDD YYRGFISETL SSIDDIDWTS LFEKMEIQLK NGDNKDTLIK | ID NO: |
| 41_20 | EQAEKRKAIY KKFADDDRFK NMFSAKLISD ILPEFVIHNN NYSASEKKEK | 174) |
| | TQVIKLFSRF ATSFKDYFKN RANCFSADDI SSSSCHRIVN DNAEIFFSNA | |
| | LVYRRIVKNL SNDDINKISG DMKDSLKEMS LEEIYSYEKY GEFITQEGIS | |
| | FYNDICGKVN SFMNLYCQKN KENKNLYKLR KLHKQILCIA DTSYEVPYKF | |
| | ESDEEVYQSV NGFLDNISSK HIVERLRKIG DNYNDYNLDK IYIVSKFYES | |
| | VSQKTYRDWE TINTALEIHY NNILPGNGKS KADKVKKAVK NDLQKSITEI | |
| | NELVSNYKLC SDDNIKAETY IHEISHILNN FEAHELKYNP EIHLVESELK | |
| | ASELKNVLDI IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HLKSSKDFDI TFCHDLIDYF KNCIAIHPEW | |
| | KNFGFDFSDT SAYEDISGFY REVELQGYKI DWTYISEKDI DLLQEKGQLY | |
| | LFQIYNKDFS KKSTGNDNLH TMYLKNLFSE ENLKDIVLKL NGEAEIFFRK | |
| | SSIKNPIIHK KGSILVNRTY EAEEKDQFGN IQIVRKTIPE NIYQELYKYF | |
| | NDKSDKELSD EAAKLKNVVG HHEAATNIVK DYRYTYDKYF LHMPITINFK | |
| | ANKTSFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPDKL KNVGHQCGCI FYVPAAYTSK IDPTTGFVNI | |
| | FKFKDLTVDA KREFIKKFDS IRYDSEKNLF CFTFDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFKLTV QMRNSLSELE DRDYDRLISP VLNENNIFYD | |
| | SAKAGYALPK DADANGAYCI ALKGLYEIKQ ITENWKEDGK FSRDKLKISN | |
| | KDWFDFIQNK RYL | |

TABLE 6
Cas12b (C2c1) orthologs

| | | |
|---|---|---|
| Alicyclobacillus | MVAVKSIKVK LMLGHLPEIR EGLWHLHEAV NLGVRYYTEW LALLRQGNLY | (SEQ |
| macrosporangiidus | RRGKDGAQEC YMTAEQCRQE LLVRLRDRQK RNGHTGDPGT DEELLGVARR | ID NO: |
| strain DSM | LYELLVPQSV GKKGQAQMLA SGFLSPLADP KSEGGKGTSK SGRKPAWMGM | 175) |
| 17980 | KEAGDSRWVE AKARYEANKA KDPTKQVIAS LEMYGLRPLF DVFTETYKTI | |
| WP_074948407.1 | RWMPLGKHQG VRAWDRDMFQ QSLERLMSWE SWNERVGAEF | |
| | ARLVDRRDRF | |
| | REKHFTGQEH LVALAQRLEQ EMKEASPGFE SKSSQAHRIT KRALRGADGI | |
| | IDDWLKLSEG EPVDRFDEIL RKRQAQNPRR FGSHDLFLKL AEPVFQPLWR | |
| | EDPSFLSRWA SYNEVLNKLE DAKQFATFTL PSPCSNPVWA RFENAEGTNI | |
| | FKYDFLFDHF GKGRHGVRFQ RMIVMRDGVP TEVEGIVVPI APSRQLDALA | |
| | PNDAASPIDV FVGDPAAPGA FRGQFGGAKI QYRRSALVRK GRREEKAYLC | |
| | GFRLPSQRRT GTPADDAGEV FLNLSLRVES QSEQAGRRNP PYAAVFHISD | |
| | QTRRVIVRYG EIERYLAEHP DTGIPGSRGL TSGLRVMSVD LGLRTSAAIS | |
| | VFRVAHRDEL TPDAHGRQPF FFPIHGMDHL VALHERSHLI RLPGETESKK | |
| | VRSIREQRLD RLNRLRSQMA SLRLLVRTGV LDEQKRDRNW ERLQSSMERG | |
| | GERMPSDWWD LFQAQVRYLA QHRDASGEAW GRMVQAAVRT | |
| | LWRQLAKQVR | |
| | DWRKEVRRNA DKVKIRGIAR DVPGGHSLAQ LDYLERQYRF LRSWSAFSVQ | |
| | AGQVVRAERD SRFAVALREH IDNGKKDRLK KLADRILMEA LGYVYVTDGR | |
| | RAGQWQAVYP PCQLVLLEEL SEYRFSNDRP PSENSQLMVW SHRGVLEELI | |
| | HQAQVHDVLV GTIPAAFSSR FDARTGAPGI RCRRVPSIPL KDAPSIPIWL | |
| | SHYLKQTERD AAALRPGELI PTGDGEFLVT PAGRGASGVR WVHADINAAH | |
| | NLQRRLWENF DLSDIRVRCD RREGKDGTW LIPRLTNQRV KERYSGVIFT | |
| | SEDGVSFTVG DAKTRRRSSA SQGEGDDLSD EEQELLAEAD DARERSVVLF | |
| | RDPSGFVNGG RWTAQRAFWG MVHNRIETLL AERFSVSGAA EKVRG | |
| Bacillus hisashii | MATRSFILKI EPNEEVKKGL WKTHEVLNHG IAYYMNILKL IRQEAIYEHH | (SEQ |
| strain C4 | EQDPKNPKKV SKAEIQAELW DFVLKMQKCN SFTHEVDKDE VFNILRELYE | ID NO: |
| WP_095142515.1 | ELVPSSVEKK GEANQLSNKF LYPLVDPNSQ SGKGTASSGR KPRWYNLKIA | 176) |
| | GDPSWEEEKK KWEEDKKKDP LAKILGKLAE YGLIPLFIPY TDSNEPIVKE | |
| | IKWMEKSRNQ SVRRLDKDMF IQALERFLSW ESWNLKVKEE YEKVEKEYKT | |
| | LEERIKEDIQ ALKALEQYEK ERQEQLLRDT LNTNEYRLSK RGLRGWREII | |
| | QKWLKMDENE PSEKYLEVFK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDFPKVVNF KPKELTEWIK | |
| | DSKGKKLKSG IESLEIGLRV MSIDLGQRQA AAASIFEVVD QKPDIEGKLF | |
| | FPIKGTELYA VHRASFNIKL PGETLVKSRE VLRKAREDNL KLMNQKLNFL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYEERS RFENSKLMKW | |
| | SRREIPRQVA LQGEIYGLQV GEVGAQFSSR FHAKTGSPGI RCSVVTKEKL | |
| | QDNRFFKNLQ REGRLTLDKI AVLKEGDLYP DKGGEKFISL SKDRKCVTTH | |
| | ADINAAQNLQ KRFWTRTHGF YKVYCKAYQV DGQTVYIPES KDQKQKIIEE | |
| | FGEGYFILKD GVYEWVNAGK LKIKKGSSKQ SSSELVDSDI LKDSFDLASE | |
| | LKGEKLMLYR DPSGNVFPSD KWMAAGVFFG KLERILISKL TNQYSISTIE | |
| | DDSSKQSM | |
| Candidatus | MPRDDLDLLT NLNSTAKGIR ERGKTKEGTD KKKSGRKSSW PMDKAAWETA | (SEQ |
| Lindowbacteria | KTSDSSAHFL EKLKQHPDLK DAFGNLSSGG SKKLEYYKKL AGSAPWKESQ | ID NO: |
| bacterium | SVILEKAARW KEAKQEREEK EQDSSEHGSK AAYRRLFDAG CLPMPEFAKY | 177) |
| RIFCSPLOWO2 | IDENQIEFGD LKLSDCGAEW KRGMWNQAGQ RVRSHMGWQR | |
| OGH55994.1 | RREKENAVYS | |
| | LRKELFEKGG AIRRKKSEEL TPEDILPGKA APDQNDWQER PAYGNQMWFI | |
| | GLRSYEENEM AKYAEEAGMG SRSAPRIRRG TIKGWSKLRE RWLQILKRNP | |
| | QATRDDLIGE LNALRSQDPR AYGDARLFDW LSKTDQRFLW DGFDADGKIL | |
| | CGRDDRDCVS AFVAYNEEFA DEPSSITLTE TDERLHPVWP FFGESSAVPY | |
| | EIEYDLETAC PTAIRLPLLV GKENGGYAER QGTRLPLAEY ADLASSFQLP | |
| | TPVRLDVLVE IREVTRAGRK VTCPFSYFKQ NGVWYVREGE IPSGESIQIK | |
| | QTDRKIENGK IFISSKLRMA YRDDLMVSPA TGDFGSIKIL WERIELASHV | |
| | DQKKLPETAP ARSRVFVSFS CNVVERAPRK QLTRKPDAVV VTIPSGVDQG | |
| | LVVVSTDVRT GKSKSSSAPP LPPGSRLWPA DAVHGDPPLR ILSVDLGHRH | |
| | SAYAVWELGL QQKSWRAGVL KGSTQTPVYA DCTGTGLLCL PGDGEDTPAE | |
| | EESLRLRSRQ IRRRLNLQNS ILRVSRLLSL DKFEKTIFEQ SDVRDRPNKK | |
| | GLRIRRRCRT EKTPLSEAEV RKNCDKAAEI LIRWADTDAM AKSLAATGNA | |
| | DISFWKYMAV KNPPLSAVVD VAPSTIVPDD GPDRETLKKK RQEEEEKFAS | |
| | SIYENRVKLA GALCSGYDAD HRRPATGGLW HDLDRTLIRE ISYGDRGQKG | |
| | NPRKLNNEGI LRLLRRPPRA RPDWREFHRT LNDANRIPKG RTLRGGLSMG | |
| | RLNFLKEVGD FVKKWSCRPR WPGDRRHIPP GQLFDRQDAE HLEHLRDDRI | |
| | KRLAHLIVAQ ALGFEPDIRR GLWKYVDGST GEILWQHPET RRFFAEGAAG | |
| | ELREVSRPAE IDDDAAARPH TVSAPAHIVV FENLIRYRFQ SDRPKTENAG | |
| | LMQWAHRQIV HFTKQVASLY GLKVAMVYAA FSSKFCSRCG SPGARVSRFD | |
| | PAWRNQEWFK RRTSNPRSKV DHSLKRASED PTADETRPWV LIEGGKEFVC | |
| | ANAKCSAHDE PLNADENAAA NIGLRFLRGV EDFRTKVNPA GALKGKLRFE | |
| | TGIHSFRPPV SGSPFWSPMA EPAQKKKIGA AAPGADVDEA GDADESGVVV | |
| | LFRDPSGAFR NKQYWYEGKI FWSNVMMAVE AKIAGASVGA KPVAASWGQA | |
| | QPQSGPGLAK PGGD | |
| Elusimicrobia | MNRIYQGRVT KVEVPDGKDE KGNIKWKKLE NWSDILWQHH MLFQDAVNYY | (SEQ |
| bacterium | TLALAAISGS AVGSDEKSII LREWAVQVQN IWEKAKKKAT VFEGPQKRLT | ID NO: |
| RIFOXYA12 | SILGLEQNAS FDIAAKHILR TSEAKPEQRA SALIRLLEEI DKKNHNVVCG | 178) |
| OGS02326.1 | ERLPFFCPRN IQSKRSPTSK AVSSVQEQKR QEEVRRFHNM QPEEVVKNAV | |
| | TLDISLFKSS PKIVFLEDPK KARAELLKQF DNACKKHKEL VGIKKAFTES | |
| | IDKHGSSLKV PAPGSKPSGL YPSAIVFKYF PVDITKTVFL KATEKLAMGK | |
| | DREVTNDPIA DARVNDKPHF DYFTNIALIR EKEKNRAAWF EFDLAAFIEA | |
| | IMSPHRFYQD TQKRKEAARK LEEKIKAIEG KGGQFKESDS EDDDVDSLPG | |
| | FEGDTRIDLL RKLVTDTLGW LGESETPDNN EGKKTEYSIS ERTLRIFPDI | |
| | QKQWSELAEK GETTEGKLLE VLKHEQTEHQ SDFGSATLYQ HLAKPEFHPI | |
| | WLKSGTEEWH AENPLKAWLN YKELQYELTD KKRPIHFTPA HPVYSPRYFD | |
| | FPKKSETEEK EVSKNTHSLT TSLASEHIKN SLQFTAGLIR KTNVGKKAIK | |
| | ARFSYSAPRL RRDCLRSENN ENLYKAPWLQ PMMRALGIDE EKADRQNFAN | |
| | TRITLMAKGL DDIQLGFPVE ANSQELQKEV SNGISWKGQF NWGGIASLSA | |
| | LRWPHEKKPK NPPEQPWWGI DSFSCLAVDL GQRYAGAFAR LDVSTIEKKG | |
| | KSRFIGEACD KKWYAKVSRM GLLRLPGEDV KVWRDASKID KENGFAFRKE | |
| | LFGEKGRSAT PLEAEETAEL IKLFGANEKD VMPDNWSKEL SFPEQNDKLL | |
| | IVARRAQAAV SRLHRWAWFF DEAKRSDDAI REILESDDTD LKQKVNKNEI | |
| | EKVKETIISL LKVKQELLPT LLTRLANRVL PLRGRSWEWK KHHQKNDGFI | |
| | LDQTGKAMPN VLIRGQRGLS MDRIEQITEL RKRFQALNQS LRRQIGKKAP | |
| | AKRDDSIPDC CPDLLEKLDH MKEQRVNQTA HMILAEALGL KLAEPPKDKK | |
| | ELNETCDMHG AYAKVDNPVS FIVIEDLSRY RSSQGRSPRE NSRLMKWCHR | |
| | AVRDKLKEMC EVFFPLCERR KAGSAWVSLP PLLETPAAYS SRFCSRSGVA | |
| | GFRAVEVIPG FELKYPWSWL KDKKDKAGNL AKEALNIRTV SEQLKAFNQD | |
| | KPEKPRTLLV PIAGGPIFVP ISEVGLSSFG LKPQVVQADI NAAINLGLRA | |
| | ISDPRIWEIH PRLRTEKRDG RLFAREKRKY GEEKVEVQPS KNEKAKKVKD | |
| | DRKPNYFADF SGKVDWGFGN IKNESGLTLV SGKALWWTIN QLQWERCFDI | |
| | NKRHIEDWSN KQKQ | |
| Omnitrophica | MNRIYQGRVT KVEKLKNGKS PDDREELKDW QTALWRHHEL FQDAVSYYTL | (SEQ |
| WOR_2 | ALAAMAEGLP DKHPINVLRK RMEEAWEEFP RKTVTPAKNL RDSVRPWLGL | ID NO: |
| bacterium | SESASFGDAL KKILPPAPEN KEVRALAVAL LAEKARTLKP QKTSASYWGR | 179) |
| RIFCSPHIGHO2 | FCDDLKKKPN WDYSEEELAR KTGSGDWVAG LWSEDALNKI DELAKSLKLS | |
| OGX36711.1 | SLVKCVPDGQ INPEGARNLV KEALDHLEGV SNGTKKEKND PGPAKKTNNW | |
| | LRQHASDVRN FIHKNKNQFS SLPNGRLITE RARGGGININ KTYAGVLFKA | |
| | FPCPFTFDYV RAAVPEPKVK KVDQEKKSEQ SATWTELEKR ILRIGDDPIE | |
| | LARKNNKPIF KAFTALEKWS DQNSKSCWSD FDKCAFEEAL KTLNQFNQKT | |
| | EEREKRRSEA EAELKYMMDE NPEWKPKKET EGDDVREVPI LKGDPRYEKL | |
| | VKLFGDLDEE GSEHATGKIY GPSRASLRGF GKLRNEWVDL FTKANDNPRE | |
| | QDLQKAVTGF QREHKLDMGY TAFFLKLCER DYWDIWRDDT EVEVKKIREK | |
| | RWVKSVVYAA ADTRELAEEL ERLQEPVRYT PAEPQFSRRL FMFSDIKGKQ | |
| | GAKHIREGLV EVSLAVKDQS GKYGTCRVRL HYSAPRLIRD HLSDGSSSMW | |
| | LQPMMAALGL SSDARGCFTR DSKGNVKEPA VALMSDFVGR KRELRMLLNF | |
| | PVDLDISKLE ENIGKKARWE KQMNTAYEKN KLKQRFHLIW PGMELKETQE | |
| | PGQFWWDNPT IQKEGMYCLA IDLSQRRAAD YALLHAGVNR DSKTFVELGQ | |
| | AGGQSWFTKL CAAGSLRLPG EDTEVIREGK RQIELSGKKG RNATQSEYDQ | |
| | AIALAKQLLH NENSAELESA ARDWLGDNAK RFSFPEQNDK LIDLYYGALS | |
| | RYKTWLRWSW RLTEQHKELW DKTLDEIRKV PYFASWGELA GNGTNEATVQ | |
| | QLQKLIADAA VDLRNFLEKA LLHIAYRALP LRENTWRWIE NGKDGKGKPL | |
| | HLLVSDGQSP AEIPWLRGQR GLSIARIEQL ENFRRAVLSL NRLLRHEIGT | |
| | KPEFGSSTCG ESLPDPCPDL TDKIVRLKEE RVNQTAHLII AQSLGVRLKG | |
| | HSLFTEEREK ADMHGEHEVI PGRSPVDFVV LEDLSRYTTD KSRSRSENSR | |
| | LMKWCHRKIN EKVKLLAEPF GIPVIEVFAS YSSKFDARTG APGFRAVEVT | |
| | SEDRPFWRKT IEKQSVAREV FDCLDNLVGK GLNGIHLVLP QNGGPLFIAA | |
| | VKEDQPLPAI RQADINAAVN IGLRAIAGPS CYHAHPKVRL IKGESGTDKG | |
| | KWLPRKGKEA NKRENAQFGN VDLDLEVKFN RLDIDSDVLK GDNTNLFHDP | |
| | LNIACYGFAT IQNLQHPFLA HASAVFSRQK GAVARLQWEV CRAINSRRLE | |
| | AWQKKAEKAA VKR | |
| Phycisphaerae | MATKSYRARI LTDSRLAAAL DRTHVVFVES LKQMINTYLR MQNGKFGPDH | (SEQ |
| bacterium ST- | KKLAQIMLSR SNTFAHGVMD QITRDQPTST LDEEWTDLAR RIHKTTGPLF | ID NO: |
| NAGAB-D1 | LQAERFATVK NRAIHTKSRG KVIPSPETLA VPAKFWHQVC DSASAYIRSN | 180) |
| (transposase) | RELMQQWRKD RAAWLKDKNE WQQKHPEFMQ FYNGPYQNFL KLCDDDRITS | |
| AQT69685.1 | QLAAEQQPTA SKNNRPRKTG KRFARWHLWY KWLSENPEII EWRNKASASD | |
| | FKTVTDDVRK QIITKYPQQN KYITRLLDWL EDNNPELKTL ENLRRTYVKK | |
| | FDSFKRPPTL TLPSPYRHPY WFTMELDQFY KKADFENGTI QLLLIDEDDD | |
| | GNWFFNWMPA SLKPDPRLVP SWRAETFETE GRFPPYLGGK IGKKLSRPAP | |
| | TDAERKAGIA GAKLMIKNNR SELLFTVFEQ DCPPRVKWAK TKNRKCPADN | |
| | AFSSDGKTRK PLRILSIDLG IRHIGAFALT QGTRNDSAWQ TESLKKGIIN | |
| | SPSIPPLRQV RRHDYDLKRK RRRHGKPVKG QRSNANLQAH RTNMAQDRFK | |
| | KGASAIVSLA REHSADLILF ENLHSLKFSA FDERWMNRQL RDMNRRHIVE | |
| | LVSEQAPEFG ITVKDDINPW MTSRICSNQN LPGFRFSMKK KNPYREKLPR | |
| | EKCTDFGYPV WEPGGHLFRC PHCDHRVNAD INAAANLANK FFGLGYWNNG | |
| | LKYDAETKTF TVHTDKKTPP LIFKPRPQFD LWADSVKTRK QLGPDPF | |
| Planctomycetes | MSVRSFQARV ECDKQTMEHL WRTHKVFNER LPEIIKILFK MKRGECGQND | (SEQ |
| bacterium | KQKSLYKSIS QSILEANAQN ADYLLNSVSI KGWKPGTAKK YRNASFTWAD | ID NO: |
| RBG_13_46_10 | DAAKLSSQGI HVYDKKQVLG DLPGMMSQMV CRQSVEAISG HIELTKKWEK | 181) |
| OHB62175.1 | EHNEWLKEKE KWESEDEHKK YLDLREKFEQ FEQSIGGKIT KRRGRWHLYL | |
| | KWLSDNPDFA AWRGNKAVIN PLSEKAQIRI NKAKPNKKNS VERDEFFKAN | |
| | PEMKALDNLH GYYERNFVRR RKTKKNPDGF DHKPTFTLPH PTIHPRWFVF | |
| | NKPKTNPEGY RKLILPKKAG DLGSLEMRLL TGEKNKGNYP DDWISVKFKA | |
| | DPRLSLIRPV KGRRVVRKGK EQGQTKETDS YEFFDKHLKK WRPAKLSGVK | |
| | LIFPDKTPKA AYLYFTCDIP DEPLTETAKK IQWLETGDVT KKGKKRKKKV | |
| | LPHGLVSCAV DLSMRRGTTG FATLCRYENG KIHILRSRNL WVGYKEGKGC | |
| | HPYRWTEGPD LGHIAKHKRE IRILRSKRGK PVKGEESHID LQKHIDYMGE | |
| | DRFKKAARTI VNFALNTENA ASKNGFYPRA DVLLLENLEG LIPDAEKERG | |
| | INRALAGWNR RHLVERVIEM AKDAGFKRRV FEIPPYGTSQ VCSKCGALGR | |
| | RYSIIRENNR REIRFGYVEK LFACPNCGYC ANADHNASVN LNRRFLIEDS | |
| | FKSYYDWKRL SEKKQKEEIE TIESKLMDKL CAMHKISRGS ISK | |
| Spirochaetes | MSFTISYPFK LIIKNKDEAK ALLDTHQYMN EGVKYYLEKL LMFRQEKIFI | (SEQ |
| bacterium | GEDETGKRIY IEETEYKKQI EEFYLIKKTE LGRNLTLTLD EFKTLMRELY | ID NO: |
| GWB1_27_13 | ICLVSSSMEN KKGFPNAQQA SLNIFSPLFD AESKGYILKE ENNNISLIHK | 182) |
| OHD16008.1 | DYGKILLKRL RDNNLIPIFT KFTDIKKITA KLSPTALDRM IFAQAIEKLL | |
| | SYESWCKLMI KERFDKEVKI KELENKCENK QERDKIFEIL EKYEEERQKT | |
| | FEQDSGFAKK GKFYITGRML KGFDEIKEKW LKEKDRSEQN LINILNKYQT | |
| | DNSKLVGDRN LFEFIIKLEN QCLWNGDIDY LKIKRDINKN QIWLDRPEMP | |
| | RFTMPDFKKH PLWYRYEDPS NSNFRNYKIE VVKDENYITI PLITERNNEY | |
| | FEENYTFNLA KLKKLSENIT FIPKSKNKEF EFIDSNDEEE DKKDQKKSKQ | |
| | YIKYCDTAKN TSYGKSGGIR LYFNRNELEN YKDGKKMDSY TVFTLSIRDY | |
| | KSLFAKEKLQ PQIFNTVDNK ITSLKIQKKF GNEEQTNFLS YFTQNQITKK | |
| | DWMDEKTFQN VKELNEGIRV LSVDLGQRFF AAVSCFEIMS EIDNNKLFFN | |
| | LNDQNHKIIR INDKNYYAKH IYSKTIKLSG EDDDLYKERK INKNYKLSYQ | |
| | ERKNKIGIFT RQINKLNQLL KIIRNDEIDK EKFKELIETT KRYVKNTYND | |
| | GIIDWNNVDN KILSYENKED VINLHKELDK KLEIDFKEFI RECRKPIFRS | |
| | GGLSMQRIDF LEKLNKLKRK WVARTQKSAE SIVLTPKFGY KLKEHINELK | |
| | DNRVKQGVNY ILMTALGYIK DNEIKNDSKK KQKEDWVKKN RACQIILMEK | |
| | LTEYTFAEDR PREENSKLRM WSHRQIFNFL QQKASLWGIL VGDVFAPYTS | |
| | KCLSDNNAPG IRCHQVTKKD LIDNSWFLKI VVKDDAFCDL IEINKENVKN | |
| | KSIKINDILP LRGGELFASI KDGKLHIVQA DINASRNIAK RFLSQINPFR | |
| | VVLKKDKDET FHLKNEPNYL KNYYSILNFV PTNEELTFFK VEENKDIKPT | |
| | KRIKMDKHEK ESTDEGDDYS KNQIALFRDD SGIFFDKSLW VDGKIFWSVV | |
| | KNKMTKLLRE RNNKKNGSK | |
| Verrucomicrobiaceae | MPLSRIYQGR TNSLIILTPT PQEPWDHKAL ARFDSPLWRH HALFQDAVNY | (SEQ |
| bacterium | YQLCLVALAS SDGTRPLSKL HEQMKASWDE AKTDTEDSWR VRLARRLGIP | ID NO: |
| UBA2429 | AASLFEAALA KVLEGNEAPE RARELAGELL LDKIEGDIQQ AGRGYWPRFC | 183) |
| GCA_002343505.1 | DPKANPTYDY SATARASASG LTKLAAVIHA ENVTEEALKQ VAAEMDLSWT | |
| | VKLQPDKNFV GAEARARLLE AAHHFIKVAE SPPTKLAEVL ARFPDGLALW | |
| | QALPEKIAAL PEETQVPRNR KASPDLTFAT LLFQHFPSLF TAAVLGLSVG | |
| | KPKSVKAPKV VEKVSARRKA NAVTQAVVIE EPEIDFAELG DDPIKLARGE | |
| | RGFVFPAFTS LSFWAVPGPH VPVWKEFDIA AFKEALKTVN QFKLKTSERN | |
| | ALLAEAQRRL DYMDEKTHDW KTGDSDEPGH IPPRLKSDPN FTLIQALTQD | |
| | EGVSNKATGD QHIPKGVYTG GLRGFYAIKK DWCELWERKA DKSQGTPTEE | |
| | ELISIVTDYQ RDHVYDVGDV GLFRALCEPR FWPLWQPLTD EQEAERIKAG | |
| | RAKDMISAYR VWLELQEDVV RLAQPIRFTP AHAENSRRLF MFSDISGSHG | |
| | AEFGSDGKSL EVSIAYDVDG KLQPVRAKLE FSAPRAARDE LEGLSGGSES | |
| | MRWFQPMMKA LDCPEVEMPA LEKCAVSLMP DVVKKGGGKW VRLLLNFPAT | |
| | LEPEGLIRHI GKQAMWYKQF NGTYKPRTQQ LDTGLHLYWP GLEKAPEAED | |
| | AAAWWNREEI RAKGFSVLSV DLGQRDAGAW ALLESRSDKA FSRNRQPFIE | |
| | LGEAGGKLWS TALLGLGMLR LPGEDARTGA LDDQGKRAVE FHGKAGRNAL | |
| | EAEWQEAREM ALLFGGEEAK SRLGPGFDHL SHSKQNEELL RILSRAQSRL | |
| | ARFHRWSCRI HEKPEATGDD VIDYGQVDEL LTKTAEAMLE NLKALYTNAG | |
| | GILDSKSKQP LTLVGLRKKL EAQKVEPEKI AAVLKPHAEI IFQRLGTLIP | |
| | ELKQHLRVSL ERLANRELPL RHREWVWNEA FEKLEQGNFK KEENPKWIRG | |
| | QRGLSMARIE QIENLRKRFM SLRRQMSLIP GEQVKQGVED KGQRQPEPCE | |
| | DILNKLDRMK QQRVNQTAHL ILAQALGLRL RPHLANDAER EEKDIHGEYE | |
| | LIPGRKPVDF IVMEDLSRYL SSQGRAPSEN GRLMKWCHRA VLAKLKQMCE | |
| | PFGIPVLEVP AAYSSRFCAL TGVPGFRAVE VHDGNAEDFR WKRLIKKAEK | |
| | DKSSKDAEAA AMLFDQLHDL NIEAREARKQ DKKLPLRTLF APVAGGPLFI | |
| | PMVGGGPRQA DMNAAINLGL RAIASPTCLR ARPKIRAELK DGKHQAMLGN | |
| | KLEKAAALTL EPPKEPTKEL AAQKRTNFFL DEKFVGKFDT AHVTTSGKKL | |
| | RLSGGMSLWK AIKDGAWQRV KKINDARIAK WKNNPPPEPD PDDEIQF | |
| Alicyclobacillus | MAVKSIKVKL RLSECPDILA GMWQLHRATN AGVRYYTEWV SLMRQEILYS | (SEQ |
| kakegawensis | RGPDGGQQCY MTAEDCQREL LRRLRNRQLH NGRQDQPGTD ADLLAISRRL | ID NO: |
| WP_067936067.1 | YEILVLQSIG KRGDAQQIAS SFLSPLVDPN SKGGRGEAKS GRKPAWQKMR | 184) |
| | DQGDPRWVAA REKYEQRKAV DPSKEILNSL DALGLRPLFA VFTETYRSGV | |
| | DWKPLGKSQG VRTWDRDMFQ QALERLMSWE SWNRRVGEEY | |
| | ARLFQQKMKF | |
| | EQEHFAEQSH LVKLARALEA DMRAASQGFE AKRGTAHQIT RRALRGADRV | |
| | FEIWKSIPEE ALFSQYDEVI RQVQAEKRRD FGSHDLFAKL AEPKYQPLWR | |
| | ADETFLTRYA LYNGVLRDLE KARQFATFTL PDACVNPIWT RFESSQGSNL | |
| | HKYEFLFDHL GPGRHAVRFQ RLLVVESEGA KERDSVVVPV APSGQLDKLV | |
| | LREEEKSSVA LHLHDTARPD GFMAEWAGAK LQYERSTLAR KARRDKQGMR | |
| | SWRRQPSMLM SAAQMLEDAK QAGDVYLNIS VRVKSPSEVR GQRRPPYAAL | |
| | FRIDDKQRRV TVNYNKLSAY LEEHPDKQIP GAPGLLSGLR VMSVDLGLRT | |
| | SASISVFRVA KKEEVEALGD GRPPHYYPIH GTDDLVAVHE RSHLIQMPGE | |
| | TETKQLRKLR EERQAVLRPL FAQLALLRLL VRCGAADERI RTRSWQRLTK | |
| | QGREFTKRLT PSWREALELE LTRLEAYCGR VPDDEWSRIV DRTVIALWRR | |
| | MGKQVRDWRK QVKSGAKVKV KGYQLDVVGG NSLAQIDYLE QQYKFLRRWS | |
| | FFARASGLVV RADRESHFAV ALRQHIENAK RDRLKKLADR ILMEALGYVY | |
| | EASGPREGQW TAQHPPCQLI ILEELSAYRF SDDRPPSENS KLMAWGHRGI | |
| | LEELVNQAQV HDVLVGTVYA AFSSRFDART GAPGVRCRRV PARFVGATVD | |
| | DSLPLWLTEF LDKHRLDKNL LRPDDVIPTG EGEFLVSPCG EEAARVRQVH | |
| | ADINAAQNLQ RRLWQNFDIT ELRLRCDVKM GGEGTVLVPR VNNARAKQLF | |
| | GKKVLVSQDG VTFFERSQTG GKPHSEKQTD LTDKELELIA EADEARAKSV | |
| | VLFRDPSGHI GKGHWIRQRE FWSLVKQRIE SHTAERIRVR GVGSSLD | |
| Bacillus sp._ V3- | MAIRSIKLKM KTNSGTDSIY LRKALWRTHQ LINEGIAYYM NLLTLYRQEA | (SEQ |
| 13 | IGDKTKEAYQ AELINIIRNQ QRNNGSSEEH GSDQEILALL RQLYELIIPS | ID NO: |
| WP_101661451.1 | SIGESGDANQ LGNKFLYPLV DPNSQSGKGT SNAGRKPRWK RLKEEGNPDW | 185) |
| | ELEKKKDEER KAKDPTVKIF DNLNKYGLLP LFPLFTNIQK DIEWLPLGKR | |
| | QSVRKWDKDM FIQAIERLLS WESWNRRVAD EYKQLKEKTE SYYKEHLTGG | |
| | EEWIEKIRKF EKERNMELEK NAFAPNDGYF ITSRQIRGWD RVYEKWSKLP | |
| | ESASPEELWK VVAEQQNKMS EGFGDPKVFS FLANRENRDI WRGHSERIYH | |
| | IAAYNGLQKK LSRTKEQATF TLPDAIEHPL WIRYESPGGT NLNLFKLEEK | |
| | QKKNYYVTLS KIIWPSEEKW IEKENIEIPL APSIQFNRQI KLKQHVKGKQ | |
| | EISFSDYSSR ISLDGVLGGS RIQFNRKYIK NHKELLGEGD IGPVFFNLVV | |
| | DVAPLQETRN GRLQSPIGKA LKVISSDFSK VIDYKPKELM DWMNTGSASN | |
| | SFGVASLLEG MRVMSIDMGQ RTSASVSIFE VVKELPKDQE QKLFYSINDT | |
| | ELFAIHKRSF LLNLPGEVVT KNNKQQRQER RKKRQFVRSQ IRMLANVLRL | |
| | ETKKTPDERK KAIHKLMEIV QSYDSWTASQ KEVWEKELNL LTNMAAFNDE | |
| | IWKESLVELH HRIEPYVGQI VSKWRKGLSE GRKNLAGISM WNIDELEDTR | |
| | RLLISWSKRS RTPGEANRIE TDEPFGSSLL QHIQNVKDDR LKQMANLIIM | |
| | TALGFKYDKE EKDRYKRWKE TYPACQIILF ENLNRYLFNL DRSRRENSRL | |
| | MKWAHRSIPR TVSMQGEMFG LQVGDVRSEY SSRFHAKTGA PGIRCHALTE | |
| | EDLKAGSNTL KRLIEDGFIN ESELAYLKKG DIIPSQGGEL FVTLSKRYKK | |
| | DSDNNELTVI HADINAAQNL QKRFWQQNSE VYRVPCQLAR MGEDKLYIPK | |
| | SQTETIKKYF GKGSFVKNNT EQEVYKWEKS EKMKIKTDTT FDLQDLDGFE | |
| | DISKTIELAQ EQQKKYLTMF RDPSGYFFNN ETWRPQKEYW SIVNNIIKSC | |
| | LKKKILSNKV EL | |
| Desulfatirhabdium | MPLSNNPPVT QRAYTLRLRG ADPSDLSWRE ALWHTHEAVN KGAKVFGDWL | (SEQ |
| butyrativorans | LTLRGGLDHT LADTKVKGGK GKPDRDPTPE ERKARRILLA LSWLSVESKL | ID NO: |
| WP_028326052.1 | GAPSSYIVAS GDEPAKDRND NVVSALEEIL QSRKVAKSEI DDWKRDCSAS | 186) |
| | LSAAIRDDAV WVNRSKVFDE AVKSVGSSLT REEAWDMLER FFGSRDAYLT | |
| | PMKDPEDKSS ETEQEDKAKD LVQKAGQWLS SRYGTSEGAD FQRMSDIYGK | |
| | IAAWADNASQ GGSSTVDDLV SELRQHFDTK ESKATNGLDW IIGLSSYTGH | |
| | TPNPVHELLR QNTSLNKSHL DDLKKKANTR AESCKSKIGS KGQRPYSDAI | |
| | LNDVESVCGF TYRVDKDGQP VSVADYSKYD VDYKWGTARH YIFAVMLDHA | |
| | ARRISLAHKW IKRAEAERHK FEEDAKRIAN VPARAREWLD SFCKERSVTS | |
| | GAVEPYRIRR RAVDGWKEVV AAWSKSDCKS TEDRIAAARA LQDDSEIDKF | |
| | GDIQLFEALA EDDALCVWHK DGEATNEPDF QPLIDYSLAI EAEFKKRQFK | |
| | VPAYRHPDEL LHPVFCDFGK SRWKINYDVH KNVQAPFYRG LCLTLWTGSE | |
| | IKPVPLCWQS KRLTRDLALG NNHRNDAASA VTRADRLGRA ASNVTKSDMV | |
| | NITGLFEQAD WNGRLQAPRQ QLEAIAVVRD NPRLSEQERN LRMCGMIEHI | |
| | RWLVTFSVKL QPQGPWCAYA EQHGLNTNPQ YWPHADTNRD RKVHARLILP | |
| | RLPGLRVLSV DLGHRYAAAC AVWEAVNTET VKEACQNVGR DMPKEHDLYL | |
| | HIKVKKQGIG KQTEVDKTTI YRRIGADTLP DGRPHPAPWA RLDRQFLIKL | |
| | QGEEKDAREA SNEEIWALHQ MECKLDRTKP LIDRLIASGW GLLKRQMARL | |
| | DALKELGWIP APDSSENLSR EDGEAKDYRE SLAVDDLMFS AVRTLRLALQ | |
| | RHGNRARIAY YLISEVKIRP GGIQEKLDEN GRIDLLQDAL ALWHELFSSP | |
| | GWRDEAAKQL WDSRIATLAG YKAPEENGDN VSDVAYRKKQ QVYREQLRNV | |
| | AKTLSGDVIT CKELSDAWKE RWEDEDQRWK KLLRWFKDWV LPSGTQANNA | |
| | TIRNVGGLSL SRLATITEFR RKVQVGFFTR LRPDGTRHEI GEQFGQKTLD | |
| | ALELLREQRV KQLASRIAEA ALGIGSEGGK GWDGGKRPRQ RINDSRFAPC | |
| | HAVVIENLAN YRPDETRTRL ENRRLMTWSA SKVHKYLSEA CQLNGLYLCT | |
| | VSAWYTSRQD SRTGAPGIRC QDVSVREFMQ SPFWRKQVKQ AEAKHDENKG | |
| | DARERFLCEL NKTWKAKTPA EWKKAGFVRI PLRGGEIFVS ADSKSPSAKG | |
| | IHADLNAAAN IGLRALTDPD WPGKWWYVPC DPVSFESKMD YVKGCAAVKV | |
| | GQPLRQPAQT NADGAASKIR KGKKNRTAGT SKEKVYLWRD ISAFPLESNE | |
| | IGEWKETSAY QNDVQYRVIR MLKEHIKSLD NRTGDNVEG | |
| Desulfonatronum | MVLGRKDDTA ELRRALWTTH EHVNLAVAEV ERVLLRCRGR SYWTLDRRGD | (SEQ |
| thiodismutans | PVHVPESQVA EDALAMAREA QRRNGWPVVG EDEEILLALR YLYEQIVPSC | ID NO: |
| WP_031386437.1 | LLDDLGKPLK GDAQKIGTNY AGPLFDSDTC RRDEGKDVAC CGPFHEVAGK | 187) |
| | YLGALPEWAT PISKQEFDGK DASHLRFKAT GGDDAFFRVS IEKANAWYED | |
| | PANQDALKNK AYNKDDWKKE KDKGISSWAV KYIQKQLQLG QDPRTEVRRK | |
| | LWLELGLLPL FIPVFDKTMV GNLWNRLAVR LALAHLLSWE SWNHRAVQDQ | |
| | ALARAKRDEL AALFLGMEDG FAGLREYELR RNESIKQHAF EPVDRPYVVS | |
| | GRALRSWTRV REEWLRHGDT QESRKNICNR LQDRLRGKFG DPDVFHWLAE | |
| | DGQEALWKER DCVTSFSLLN DADGLLEKRK GYALMTFADA RLHPRWAMYE | |
| | APGGSNLRTY QIRKTENGLW ADVVLLSPRN ESAAVEEKTF NVRLAPSGQL | |
| | SNVSFDQIQK GSKMVGRCRY QSANQQFEGL LGGAEILFDR KRIANEQHGA | |
| | TDLASKPGHV WFKLTLDVRP QAPQGWLDGK GRPALPPEAK HFKTALSNKS | |
| | KFADQVRPGL RVLSVDLGVR SFAACSVFEL VRGGPDQGTY FPAADGRTVD | |
| | DPEKLWAKHE RSFKITLPGE NPSRKEEIAR RAAMEELRSL NGDIRRLKAI | |
| | LRLSVLQEDD PRTEHLRLFM EAIVDDPAKS ALNAELFKGF GDDRFRSTPD | |
| | LWKQHCHFFH DKAEKWVAER FSRWRTETRP KSSSWQDWRE | |
| | RRGYAGGKSY | |
| | WAVTYLEAVR GLILRWNMRG RTYGEVNRQD KKQFGTVASA LLHHINQLKE | |
| | DRIKTGADMI IQAARGFVPR KNGAGWVQVH EPCRLILFED LARYRFRTDR | |
| | SRRENSRLMR WSHREIVNEV GMQGELYGLH VDTTEAGFSS RYLASSGAPG | |
| | VRCRHLVEED FHDGLPGMHL VGELDWLLPK DKDRTANEAR RLLGGMVRPG | |
| | MLVPWDGGEL FATLNAASQL HVIHADINAA QNLQRRFWGR CGEAIRIVCN | |
| | QLSVDGSTRY EMAKAPKARL LGALQQLKNG DAPFHLTSIP NSQKPENSYV | |
| | MTPTNAGKKY RAGPGEKSSG EEDELALDIV EQAEELAQGR KTFFRDPSGV | |
| | FFAPDRWLPS EIYWSRIRRR IWQVTLERNS SGRQERAEMD EMPY | |
| Lentisphaeria | MAVELNRIYQ GRVNHVYIFD ENQNQVSVDN GDDLLFVHHE LYQDAINYYL | (SEQ |
| bacterium | VALAAMALDS KDSLFGKFKM QIRAVWNDFY RNGQLRPGLK HSLIRSLGHA | ID NO: |
| DCFZ01000012.1 | AELNTSNGAD IAMNLILEDG GIPSEILNAA LEHLAEKCTG DVSQLGKTFF | 188) |
| | PRFCDTAYHG NWDVDAKSFS EKKGRQRLVD ALYSLHPVQA VQELAPEIEI | |
| | GWGGVKTQTG KFFTGDEAKA SLKKAISYFL QDTGKNSPEL QEYFSVAGKQ | |
| | PLEQYLGKID TFPEISFGRI SSHQNINISN AMWILKFFPD QYSVDLIKNL | |
| | IPNKKYEIGI APQWGDDPVK LSRGKRGYTF RAFTDLAMWE KNWKVFDRAA | |
| | FSDALKTINQ FRNKTQERND QLKRYCAALN WMDGESSDKK PPVEPADADA | |
| | VDEAATSVLP ILAGDKRWNA LLQLQKELGI CNDFTENELM DYGLSLRTIR | |
| | GYQKLRSMML EKEEKMRAKT ADDEEISQAL QEIIIKFQSS HRDTIGSVSL | |
| | FLKLAEPKYF CVWHDADKNQ NFASVDMVAD AVRYYSYQEE KARLEEPIQI | |
| | TPADARYSRR VSDLYALVYK NAKECKTGYG LRPDGNFVFE IAQKNAKGYA | |
| | PAKWVLAFSA PRLKRDGLID KEFSAYYPPV LQAFLREEEA PKQSFKTTAV | |
| | ILMPDWDKNG KRRILLNFPI KLDVSAIHQK TDHRFENQFY FANNTNTCLL | |
| | WPSYQYKKPV TWYQGKKPFD VVAVDLGQRS AGAVSRITVS TEKREHSVAI | |
| | GEAGGTQWYA YRKESGLLRL PGEDATVIRD GQRTEELSGN AGRLSTEEET | |
| | VQACVLCKML IGDATLLGGS DEKTIRSFPK QNDKLLIAFR RATGRMKQLQ | |
| | RWLWMLNENG LCDKAKTEIS NSDWLVNKNI DNVLKEEKQH REMLPAILLQ | |
| | IADRVLPLRG RKWDWVLNPQ SNSFVLQQTA HGSGDPHKKI CGQRGLSFAR | |
| | IEQLESLRMR CQALNRILMR KTGEKPATLA EMRNNPIPDC CPDILMRLDA | |
| | MKEQRINQTA NLILAQALGL RHCLHSESAT KRKENGMHGE YEKIPGVEPA | |
| | AFVVLEDLSR YRFSQDRSSY ENSRLMKWSH RKILEKLALL CEVFNVPILQ | |
| | VGAAYSSKFS ANAIPGFRAE ECSIDQLSFY PWRELKDSRE KALVEQIRKI | |
| | GHRLLTFDAK ATIIMPRNGG PVFIPFVPSD SKDTLIQADI NASFNIGLRG | |
| | VADATNLLCN NRVSCDRKKD CWQVKRSSNF SKMVYPEKLS LSFDPIKKQE | |
| | GAGGNFFVLG CSERILTGTS EKSPVFTSSE MAKKYPNLMF GSALWRNEIL | |
| | KLERCCKINQ SRLDKFIAKK EVQNEL | |
| Laceyella | MSIRSFKLKI KTKSGVNAEE LRRGLWRTHQ LINDGIAYYM NWLVLLRQED | (SEQ |
| sediminis | LFIRNEETNE IEKRSKEEIQ GELLERVHKQ QQRNQWSGEV DDQTLLQTLR | ID NO: |
| WP_106341859.1 | HLYEEIVPSV IGKSGNASLK ARFFLGPLVD PNNKTTKDVS KSGPTPKWKK | 189) |
| | MKDAGDPNWV QEYEKYMAER QTLVRLEEMG LIPLFPMYTD EVGDIHWLPQ | |
| | ASGYTRTWDR DMFQQAIERL LSWESWNRRV RERRAQFEKK THDFASRFSE | |
| | SDVQWMNKLR EYEAQQEKSL EENAFAPNEP YALTKKALRG WERVYHSWMR | |
| | LDSAASEEAY WQEVATCQTA MRGEFGDPAI YQFLAQKENH DIWRGYPERV | |
| | IDFAELNHLQ RELRRAKEDA TFTLPDSVDH PLWVRYEAPG GTNIHGYDLV | |
| | QDTKRNLTLI LDKFILPDEN GSWHEVKKVP FSLAKSKQFH RQVWLQEEQK | |
| | QKKREVVFYD YSTNLPHLGT LAGAKLQWDR NFLNKRTQQQ IEETGEIGKV | |
| | FFNISVDVRP AVEVKNGRLQ NGLGKALTVL THPDGTKIVT GWKAEQLEKW | |
| | VGESGRVSSL GLDSLSEGLR VMSIDLGQRT SATVSVFEIT KEAPDNPYKF | |
| | FYQLEGTELF AVHQRSFLLA LPGENPPQKI KQMREIRWKE RNRIKQQVDQ | |
| | LSAILRLHKK VNEDERIQAI DKLLQKVASW QLNEEIATAW NQALSQLYSK | |
| | AKENDLQWNQ AIKNAHHQLE PVVGKQISLW RKDLSTGRQG IAGLSLWSIE | |
| | ELEATKKLLT RWSKRSREPG VVKRIERFET FAKQIQHHIN QVKENRLKQL | |
| | ANLIVMTALG YKYDQEQKKW IEVYPACQWV LFENLRSYRF SYERSRRENK | |
| | KLMEWSHRSI PKLVQMQGEL FGLQVADVYA AYSSRYHGRT GAPGIRCHAL | |
| | TEADLRNETN IIHELIEAGF IKEEHRPYLQ QGDLVPWSGG ELFATLQKPY | |
| | DNPRILTLHA DINAAQNIQK RFWHPSMWFR VNCESVMEGE IVTYVPKNKT | |
| | VHKKQGKTFR FVKVEGSDVY EWAKWSKNRN KNTFSSITER KPPSSMILFR | |
| | DPSGTFFKEQ EWVEQKTFWG KVQSMIQAYM KKTIVQRMEE | |
| Methylobacterium | MYEAIVLADD ANAQLANAFL GPLTDPNSAG FLEAFNKVDR PAPSWLDQVP | (SEQ |
| nodulans (long | ASDPIDPAVL AEANAWLDTD AGRAWLVDTG APPRWRSLAA KQDPIWPREF | ID NO: |
| form) | ARKLGELRKE AASGTSAIIK ALKRDFGVLP LFQPSLAPRI LGSRSSLTPW | 190) |
| | DRLAFRLAVG HLLSWESWCT RARDEHTARV QRLEQFSSAH LKGDLATKVS | |
| | TLREYERARK EQIAQLGLPM GERDFLITVR MTRGWDDLRE KWRRSGDKGQ | |
| | EALHAIIATE QTRKRGRFGD PDLFRWLARP ENHHVWADGH ADAVGVLARV | |
| | NAMERLVERS RDTALMTLPD PVAHPRSAQW EAEGGSNLRN YQLEAVGGEL | |
| | QITLPLLKAA DDGRCIDTPL SFSLAPSDQL QGVVLTKQDK QQKITYCTNM | |
| | NEVFEAKLGS ADLLLNWDHL RGRIRDRVDA GDIGSAFLKL ALDVAHVLPD | |
| | GVDDQLARAA FHFQSAKGAK SKHADSVQAG LRVLSIDLGV RSFATCSVFE | |
| | LKDTAPTTGV AFPLAEFRLW AVHERSFTLE LPGENVGAAG QQWRAQADAE | |
| | LRQLRGGLNR HRQLLRAATV QKGERDAYLT DLREAWSAKE LWPFEASLLS | |
| | ELERCSTVAD PLWQDTCKRA ARLYRTEFGA VVSEWRSRTR SREDRKYAGK | |
| | SMWSVQHLTD VRRFLQSWSL AGRASGDIRR LDRERGGVFA KDLLDHIDAL | |
| | KDDRLKTGAD LIVQAARGFQ RNEFGYWVQK HAPCHVILFE DLSRYRMRTD | |
| | RPRRENSQLM QWAHRGVPDM VGMQGEIYGI QDRRDPDSAR KHARQPLAAF | |
| | CLDTPAAFSS RYHASTMTPG IRCHPLRKRE FEDQGFLELL KRENEGLDLN | |
| | GYKPGDLVPL PGGEVFVCLN ANGLSRIHAD INAAQNLQRR FWTQHGDAFR | |
| | LPCGKSAVQG QIRWAPLSMG KRQAGALGGF GYLEPTGHDS GSCQWRKTTE | |
| | AEWRRLSGAQ KDRDEAAAAE DEELQGLEEE LLERSGERVV FFRDPSGVVL | |
| | PTDLWFPSAA FWSIVRAKTV GRLRSHLDAQ AEASYAVAAG L | |
| Opitutaceae | MSLNRIYQGR VAAVETGTAL AKGNVEWMPA AGGDEVLWQH HELFQAAINY | (SEQ |
| bacterium | YLVALLALAD KNNPVLGPLI SQMDNPQSPY HVWGSFRRQG RQRTGLSQAV | ID NO: |
| WP_009513281.1 | APYITPGNNA PTLDEVFRSI LAGNPTDRAT LDAALMQLLK ACDGAGAIQQ | 191) |
| | EGRSYWPKFC DPDSTANFAG DPAMLRREQH RLLLPQVLHD PAITHDSPAL | |
| | GSFDTYSIAT PDTRTPQLTG PKARARLEQA ITLWRVRLPE SAADFDRLAS | |
| | SLKKIPDDDS RLNLQGYVGS SAKGEVQARL FALLLFRHLE RSSFTLGLLR | |
| | SATPPPKNAE TPPPAGVPLP AASAADPVRI ARGKRSFVFR AFTSLPCWHG | |
| | GDNIHPTWKS FDIAAFKYAL TVINQIEEKT KERQKECAEL ETDFDYMHGR | |
| | LAKIPVKYTT GEAEPPPILA NDLRIPLLRE LLQNIKVDTA LTDGEAVSYG | |
| | LQRRTIRGFR ELRRIWRGHA PAGTVFSSEL KEKLAGELRQ FQTDNSTTIG | |
| | SVQLFNELIQ NPKYWPIWQA PDVETARQWA DAGFADDPLA ALVQEAELQE | |
| | DIDALKAPVK LTPADPEYSR RQYDFNAVSK FGAGSRSANR HEPGQTERGH | |
| | NTFTTEIAAR NAADGNRWRA THVRIHYSAP RLLRDGLRRP DTDGNEALEA | |
| | VPWLQPMMEA LAPLPTLPQD LTGMPVFLMP DVTLSGERRI LLNLPVTLEP | |
| | AALVEQLGNA GRWQNQFFGS REDPFALRWP ADGAVKTAKG KTHIPWHQDR | |
| | DHFTVLGVDL GTRDAGALAL LNVTAQKPAK PVHRIIGEAD GRTWYASLAD | |
| | ARMIRLPGED ARLFVRGKLV QEPYGERGRN ASLLEWEDAR NIILRLGQNP | |
| | DELLGADPRR HSYPEINDKL LVALRRAQAR LARLQNRSWR LRDLAESDKA | |
| | LDEIHAERAG EKPSPLPPLA RDDAIKSTDE ALLSQRDIIR RSFVQIANLI | |
| | LPLRGRRWEW RPHVEVPDCH ILAQSDPGTD DTKRLVAGQR GISHERIEQI | |
| | EELRRRCQSL NRALRHKPGE RPVLGRPAKG EEIADPCPAL LEKINRLRDQ | |
| | RVDQTAHAIL AAALGVRLRA PSKDRAERRH RDIHGEYERF RAPADFVVIE | |
| | NLSRYLSSQD RARSENTRLM QWCHRQIVQK LRQLCETYGI PVLAVPAAYS | |
| | SRFSSRDGSA GFRAVHLTPD HRHRMPWSRI LARLKAHEED GKRLEKTVLD | |
| | EARAVRGLFD RLDRFNAGHV PGKPWRTLLA PLPGGPVFVP LGDATPMQAD | |
| | LNAAINIALR GIAAPDRHDI HHRLRAENKK RILSLRLGTQ REKARWPGGA | |
| | PAVTLSTPNN GASPEDSDAL PERVSNLFVD IAGVANFERV TIEGVSQKFA | |
| | TGRGLWASVK QRAWNRVARL NETVTDNNRN EEEDDIPM | |
| Thermomonas | MSEKTTQRAY TLRLNRASGE CAVCQNNSCD CWHDALWATH KAVNRGAKAF | (SEQ |
| hydrothermalis | GDWLLTLRGG LCHTLVEMEV PAKGNNPPQR PTDQERRDRR VLLALSWLSV | ID NO: |
| WP_072754838.1 | EDEHGAPKEF IVATGRDSAD DRAKKVEEKL REILEKRDFQ EHEIDAWLQD | 192) |
| | CGPSLKAHIR EDAVWVNRRA LFDAAVERIK TLTWEEAWDF LEPFFGTQYF | |
| | AGIGDGKDKD DAEGPARQGE KAKDLVQKAG QWLSARFGIG TGADFMSMAE | |
| | AYEKIAKWAS QAQNGDNGKA TIEKLACALR PSEPPTLDTV LKCISGPGHK | |
| | SATREYLKTL DKKSTVTQED LNQLRKLADE DARNCRKKVG KKGKKPWADE | |
| | VLKDVENSCE LTYLQDNSPA RHREFSVMLD HAARRVSMAH SWIKKAEQRR | |
| | RQFESDAQKL KNLQERAPSA VEWLDRFCES RSMTTGANTG SGYRIRKRAI | |
| | EGWSYVVQAW AEASCDTEDK RIAAARKVQA DPEIEKFGDI QLFEALAADE | |
| | AICVWRDQEG TQNPSILIDY VTGKTAEHNQ KRFKVPAYRH PDELRHPVFC | |
| | DFGNSRWSIQ FAIHKEIRDR DKGAKQDTRQ LQNRHGLKMR LWNGRSMTDV | |
| | NLHWSSKRLT ADLALDQNPN PNPTEVTRAD RLGRAASSAF DHVKIKNVFN | |
| | EKEWNGRLQA PRAELDRIAK LEEQGKTEQA EKLRKRLRWY VSFSPCLSPS | |
| | GPFIVYAGQH NIQPKRSGQY APHAQANKGR ARLAQLILSR LPDLRILSVD | |
| | LGHRFAAACA VWETLSSDAF RREIQGLNVL AGGSGEGDLF LHVEMTGDDG | |
| | KRRTVVYRRI GPDQLLDNTP HPAPWARLDR QFLIKLQGED EGVREASNEE | |
| | LWTVHKLEVE VGRTVPLIDR MVRSGFGKTE KQKERLKKLR ELGWISAMPN | |
| | EPSAETDEKE GEIRSISRSV DELMSSALGT LRLALKRHGN RARIAFAMTA | |
| | DYKPMPGGQK YYFHEAKEAS KNDDETKRRD NQIEFLQDAL SLWHDLFSSP | |
| | DWEDNEAKKL WQNHIATLPN YQTPEEISAE LKRVERNKKR KENRDKLRTA | |
| | AKALAENDQL RQHLHDTWKE RWESDDQQWK ERLRSLKDWI FPRGKAEDNP | |
| | SIRHVGGLSI TRINTISGLY QILKAFKMRP EPDDLRKNIP QKGDDELENF | |
| | NRRLLEARDR LREQRVKQLA SRIIEAALGV GRIKIPKNGK LPKRPRTTVD | |
| | TPCHAWVIES LKTYRPDDLR TRRENRQLMQ WSSAKVRKYL KEGCELYGLH | |
| | FLEVPANYTS RQCSRTGLPG IRCDDVPTGD FLKAPWWRRA INTAREKNGG | |
| | DAKDRFLVDL YDHLNNLQSK GEALPATVRV PRQGGNLFIA GAQLDDTNKE | |
| | RRAIQADLNA AANIGLRALL DPDWRGRWWY VPCKDGTSEP ALDRIEGSTA | |
| | FNDVRSLPTG DNSSRRAPRE IENLWRDPSG DSLESGTWSP TRAYWDTVQS | |
| | RVIELLRRHA GLPTS | |
| Methylobacterium | MYEAIVLADD ANAQLANAFL GPLTDPNSAG FLEAFNKVDR PAPSWLDQVP | (SEQ |
| nodulans | ASDPIDPAVL AEANAWLDTD AGRAWLVDTG APPRWRSLAA KQDPIWPREF | ID NO: |
| WP_043747912.1 | ARKLGELRKE AASGTSAIIK ALKRDFGVLP LFQPSLAPRI LGSRSSLTPW | 193) |
| | DRLAFRLAVG HLLSWESWCT RARDEHTARV QRLEQFSSAH LKGDLATKVS | |
| | TLREYERARK EQIAQLGLPM GERDFLITVR MTRGWDDLRE KWRRSGDKGQ | |
| | EALHAIIATE QTRKRGRFGD PDLFRWLARP ENHHVWADGH ADAVGVLARV | |
| | NAMERLVERS RDTALMTLPD PVAHPRSAQW EAEGGSNLRN YQLEAVGGEL | |
| | QITLPLLKAA DDGRCIDTPL | |
| Chloracidobacterium | MPQQAKPPVT QRAYTLRLRG ADSNDPSWRD ALWQTHEAVN | (SEQ |
| thermophilum | RGAQAFGDWL | ID NO: |
| WP_058868187.1 | LTLRGGLDHT LADTPVKGGK GKPDPDPTDE ERKARRILLA LSWLSVESKL | 194) |
| | GAPAGLIIAF GTEAAEERNR KWVAALEEIL KSRGVDQNEI NAWKKDCSAS | |
| | LSAAIRDDAV WVNRSKAFDE AVESIGSSGS SGSSLTREEP WDMLERFFGS | |
| | RDAYLAPAKG SEDESSEAKQ EDQAKDLVQK AGQWLSSRFG TGKGADFRRM | |
| | ATVYEAIAKW DGKASLEMAG DKAIADLATA LSEFNPASND LQGVLGLISG | |
| | PGYKSATRNF LNQLAAQTTV TQQDFVSLKD KANNDAQECK QNTGSKGQRP | |
| | YSNSILEKVE SVCGFTYLQD GGPARHSEFA VILDHAARRV SLAHTWIKLA | |
| | EAERRKFEED AKKIDQVPEA AKDWLDRFCL ERSGVSGALE PYRIRRRAVD | |
| | GWKEVVAEWS KSDCKTVEDR IAAARALQDD PEIDKFGDIQ LFEALAEDDA | |
| | VCVWHKDGDA AKAPDPQPLI DYALAAEAEF KKRHFKVPAY RHPDALLHPI | |
| | FCDFGKSRWD ICFDVHKNMQ TPFPRALCLT LWTGSEMKRI PLCWQSKRLA | |
| | RDLALGNNTG DAGASEVTRA DRLGRAASRA ASNVTKSDVV NIAGLFEQAD | |
| | WNGRLQAPRQ QLEAIARYVE KHDWDQKAEK MRNAIQWLVT FSARLQPQGP | |
| | WCAYAKIHGL KEDPQYWPHA DTNKNRKGHA RLILSRLPGL RVLAVDLGHR | |
| | YAAACAVWEA LSTEAFQREI KGRTILRGRT DGNALYCHTR HKANGKERVT | |
| | IYRRIGADTL PDGKPHPAPW ARLDRQFLIK LQGEEEGVRE ASNEEIWAVH | |
| | QLEAALGRPV SLIDRLVASG WGGSDKQKAR LEGLKQLGWD PADKPSLSVD | |
| | ELMSSAVRTM RLALKRHGDR ARIAHYLITD EKTTPGGIKE TLDEKGRIDL | |
| | LQDALVLWHD LFSSRGWRDD TAKQLWNAHV AKLHGYKAPE EPGEDSSGAE | |
| | RKKKQRENRE KLYDVAKALA QDVTLREALH DAWKKRWEND DERWKKQLRW | |
| | FKDWVFPRGN HASDPTIRKR QLINPSGGNG RRGNHASDPT IRKRQLINPS | |
| | GGNGRRGNHA SDPTIRKVGG LSLPRLATLT EFRRKVQVGF FTRLKPDGTR | |
| | AETKEQFGQS ALDALEHLRE QRVKQLASRI AEAALGVGRV RRPVEGKDPK | |
| | RPDVRVDEPC HAIVIEDLTH YRPEETRTRR ENRQLMTWSS SKVKKYLAEA | |
| | CQLHGLHLRE VSASYTSRQD SRTGAPGVRC QDVPVKEFMR SPFWRKQVKQ | |
| | AEAKQAANKG DARERLLCDL NARWKDRTAA DWEKAGAVRI PLQGGEIFVS | |
| | ADANSPAAKG IQADLNAAAN IGLRALTDPD WAGKWWYVPC DPASFRPVRD | |
| | KVDGSAVVNP DQPLRQSAQA QSGDAAKDKN GNKGAGKSKE VVNLWRDISS | |
| | SPLECIEFGE WKEYAAYQNE VQCRVIRILK EQIKGRDKQP HEGSKEDDIP | |
| | L | |
| Desulfovibrio | MPTRTINLKL VLGKNPENAT LRRALFSTHR LVNQATKRIE EFLLLCRGEA | (SEQ |
| inopinatus | YRTVDNEGKE AEIPRHAVQE EALAFAKAAQ RHNGCISTYE DQEILDVLRQ | ID NO: |
| WP_027186183.1 | LYERLVPSVN ENNEAGDAQA ANAWVSPLMS AESEGGLSVY DKVLDPPPVW | 195) |
| | MKLKEEKAPG WEAASQIWIQ SDEGQSLLNK PGSPPRWIRK LRSGQPWQDD | |
| | FVSDQKKKQD ELTKGNAPLI KQLKEMGLLP LVNPFFRHLL DPEGKGVSPW | |
| | DRLAVRAAVA HFISWESWNH RTRAEYNSLK LRRDEFEAAS DEFKDDFTLL | |
| | RQYEAKRHST LKSIALADDS NPYRIGVRSL RAWNRVREEW IDKGATEEQR | |
| | VTILSKLQTQ LRGKFGDPDL FNWLAQDRHV HLWSPRDSVT PLVRINAVDK | |
| | VLRRRKPYAL MTFAHPRFHP RWILYEAPGG SNLRQYALDC TENALHITLP | |
| | LLVDDAHGTW IEKKIRVPLA PSGQIQDLTL EKLEKKKNRL YYRSGFQQFA | |
| | GLAGGAEVLF HRPYMEHDER SEESLLERPG AVWFKLTLDV ATQAPPNWLD | |
| | GKGRVRTPPE VHHFKTALSN KSKHTRTLQP GLRVLSVDLG MRTFASCSVF | |
| | ELIEGKPETG RAFPVADERS MDSPNKLWAK HERSFKLTLP GETPSRKEEE | |
| | ERSIARAEIY ALKRDIQRLK SLLRLGEEDN DNRRDALLEQ FFKGWGEEDV | |
| | VPGQAFPRSL FQGLGAAPFR STPELWRQHC QTYYDKAEAC LAKHISDWRK | |
| | RTRPRPTSRE MWYKTRSYHG GKSIWMLEYL DAVRKLLLSW SLRGRTYGAI | |
| | NRQDTARFGS LASRLLHHIN SLKEDRIKTG ADSIVQAARG YIPLPHGKGW | |
| | EQRYEPCQLI LFEDLARYRF RVDRPRRENS QLMQWNHRAI VAETTMQAEL | |
| | YGQIVENTAA GFSSRFHAAT GAPGVRCRFL LERDFDNDLP KPYLLRELSW | |
| | MLGNTKVESE EEKLRLLSEK IRPGSLVPWD GGEQFATLHP KRQTLCVIHA | |
| | DMNAAQNLQR RFFGRCGEAF RLVCQPHGDD VLRLASTPGA RLLGALQQLE | |
| | NGQGAFELVR DMGSTSQMNR FVMKSLGKKK IKPLQDNNGD DELEDVLSVL | |
| | PEEDDTGRIT VFRDSSGIFF PCNVWIPAKQ FWPAVRAMIW KVMASHSLG | |
| Desulfonatronum | MVLGRKDDTA ELRRALWTTH EHVNLAVAEV ERVLLRCRGR SYWTLDRRGD | (SEQ |
| thiodismutans | PVHVPESQVA EDALAMAREA QRRNGWPVVG EDEEILLALR YLYEQIVPSC | ID NO: |
| WP_031386437.1 | LLDDLGKPLK GDAQKIGTNY AGPLFDSDTC RRDEGKDVAC CGPFHEVAGK | 187) |
| | YLGALPEWAT PISKQEFDGK DASHLRFKAT GGDDAFFRVS IEKANAWYED | |
| | PANQDALKNK AYNKDDWKKE KDKGISSWAV KYIQKQLQLG QDPRTEVRRK | |
| | LWLELGLLPL FIPVFDKTMV GNLWNRLAVR LALAHLLSWE SWNHRAVQDQ | |
| | ALARAKRDEL AALFLGMEDG FAGLREYELR RNESIKQHAF EPVDRPYVVS | |
| | GRALRSWTRV REEWLRHGDT QESRKNICNR LQDRLRGKFG DPDVFHWLAE | |
| | DGQEALWKER DCVTSFSLLN DADGLLEKRK GYALMTFADA RLHPRWAMYE | |
| | APGGSNLRTY QIRKTENGLW ADVVLLSPRN ESAAVEEKTF NVRLAPSGQL | |
| | SNVSFDQIQK GSKMVGRCRY QSANQQFEGL LGGAEILFDR KRIANEQHGA | |
| | TDLASKPGHV WFKLTLDVRP QAPQGWLDGK GRPALPPEAK HFKTALSNKS | |
| | KFADQVRPGL RVLSVDLGVR SFAACSVFEL VRGGPDQGTY FPAADGRTVD | |
| | DPEKLWAKHE RSFKITLPGE NPSRKEEIAR RAAMEELRSL NGDIRRLKAI | |
| | LRLSVLQEDD PRTEHLRLFM EAIVDDPAKS ALNAELFKGF GDDRFRSTPD | |
| | LWKQHCHFFH DKAEKVVAER FSRWRTETRP KSSSWQDWRE | |
| | RRGYAGGKSY | |
| | WAVTYLEAVR GLILRWNMRG RTYGEVNRQD KKQFGTVASA LLHHINQLKE | |
| | DRIKTGADMI IQAARGFVPR KNGAGWVQVH EPCRLILFED LARYRFRTDR | |
| | SRRENSRLMR WSHREIVNEV GMQGELYGLH VDTTEAGFSS RYLASSGAPG | |
| | VRCRHLVEED FHDGLPGMHL VGELDWLLPK DKDRTANEAR RLLGGMVRPG | |
| | MLVPWDGGEL FATLNAASQL HVIHADINAA QNLQRRFWGR CGEAIRIVCN | |
| | QLSVDGSTRY EMAKAPKARL LGALQQLKNG DAPFHLTSIP NSQKPENSYV | |
| | MTPTNAGKKY RAGPGEKSSG EEDELALDIV EQAEELAQGR KTFFRDPSGV | |
| | FFAPDRWLPS EIYWSRIRRR IWQVTLERNS SGRQERAEMD EMPY | |
| Tuberibacillus | MATKSFILKM KTKNNPQLRL SLWKTHELFN FGVAYYMDLL SLFRQKDLYM | (SEQ |
| calidus | HNDEDPDHPV VLKKEEIQER LWMKVRETQQ KNGFHGEVSK DEVLETLRAL | ID NO: |
| WP_027726362.1 | YEELVPSAVG KSGEANQISN KYLYPLTDPA SQSGKGTANS GRKPRWKKLK | 196) |
| | EAGDPSWKDA YEKWEKERQE DPKLKILAAL QSFGLIPLFR PFTENDHKAV | |
| | ISVKWMPKSK NQSVRKFDKD MFNQAIERFL SWESWNEKVA EDYEKTVSIY | |
| | ESLQKELKGI STKAFEIMER VEKAYEAHLR EITFSNSTYR IGNRAIRGWT | |
| | EIVKKWMKLD PSAPQGNYLD VVKDYQRRHP RESGDFKLFE LLSRPENQAA | |
| | WREYPEFLPL YVKYRHAEQR MKTAKKQATF TLCDPIRHPL WVRYEERSGT | |
| | NLNKYRLIMN EKEKVVQFDR LICLNADGHY EEQEDVTVPL APSQQFDDQI | |
| | KFSSEDTGKG KHNFSYYHKG INYELKGTLG GARIQFDREH LLRRQGVKAG | |
| | NVGRIFLNVT LNIEPMQPFS RSGNLQTSVG KALKVYVDGY PKWVNFKPKE | |
| | LTEHIKESEK NTLTLGVESL PTGLRVMSVD LGQRQAAAIS IFEVVSEKPD | |
| | DNKLFYPVKD TDLFAVHRTS FNIKLPGEKR TERRMLEQQK RDQAIRDLSR | |
| | KLKFLKNVLN MQKLEKTDER EKRVNRWIKD REREEENPVY VQEFEMISKV | |
| | LYSPHSVWVD QLKSIHRKLE EQLGKEISKW RQSISQGRQG VYGISLKNIE | |
| | DIEKTRRLLF RWSMRPENPG EVKQLQPGER FAIDQQNHLN HLKDDRIKKL | |
| | ANQIVMTALG YRYDGKRKKW IAKHPACQLV LFEDLSRYAF YDERSRLENR | |
| | NLMRWSRREI PKQVAQIGGL YGLLVGEVGA QYSSRFHAKS GAPGIRCRVV | |
| | KEHELYITEG GQKVRNQKFL DSLVENNIIE PDDARRLEPG DLIRDQGGDK | |
| | FATLDERGEL VITHADINAA QNLQKRFWTR THGLYRIRCE SREIKDAWVL | |
| | VPSDKDQKEK MENLFGIGYL QPFKQENDVY KWVKGEKIKG KKTSSQSDDK | |
| | ELVSEILQEA SVMADELKGN RKTLFRDPSG YVFPKDRWYT GGRYFGTLEH | |
| | LLKRKLAERR LFDGGSSRRG LFNGTDSNTN VE | |
| Bacillus | MATRSFILKI EPNEEVKKGL WKTHEVLNHG IAYYMNILKL IRQEAIYEHH | (SEQ |
| thermoamylovorans | EQDPKNPKKV SKAEIQAELW DFVLKMQKCN SFTHEVDKDV VFNILRELYE | ID NO: |
| WP_041902512.1 | ELVPSSVEKK GEANQLSNKF LYPLVDPNSQ SGKGTASSGR KPRWYNLKIA | 197) |
| | GDPSWEEEKK KWEEDKKKDP LAKILGKLAE YGLIPLFIPF TDSNEPIVKE | |
| | IKWMEKSRNQ SVRRLDKDMF IQALERFLSW ESWNLKVKEE YEKVEKEHKT | |
| | LEERIKEDIQ AFKSLEQYEK ERQEQLLRDT LNTNEYRLSK RGLRGWREII | |
| | QKWLKMDENE PSEKYLEVFK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDFPKFVNF KPKELTEWIK | |
| | DSKGKKLKSG IESLEIGLRV MSIDLGQRQA AAASIFEVVD QKPDIEGKLF | |
| | FPIKGTELYA VHRASFNIKL PGETLVKSRE VLRKAREDNL KLMNQKLNFL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYEERS RFENSKLMKW | |
| | SRREIPRQVA LQGEIYGLQV GEVGAQFSSR FHAKTGSPGI RCSVVTKEKL | |
| | QDNRFFKNLQ REGRLTLDKI AVLKEGDLYP DKGGEKFISL SKDRKLVTTH | |
| | ADINAAQNLQ KRFWTRTHGF YKVYCKAYQV DGQTVYIPES KDQKQKIIEE | |
| | FGEGYFILKD GVYEWGNAGK LKIKKGSSKQ SSSELVDSDI LKDSFDLASE | |
| | LKGEKLMLYR DPSGNVFPSD KWMAAGVFFG KLERILISKL TNQYSISTIE | |
| | DDSSKQSM | |
| Bacillus sp. | MAIRSIKLKL KTHTGPEAQN LRKGIWRTHR LLNEGVAYYM KMLLLFRQES | (SEQ |
| NSP2.1 | TGERPKEELQ EELICHIREQ QQRNQADKNT QALPLDKALE ALRQLYELLV | ID NO: |
| WP_026557978.1 | PSSVGQSGDA QIISRKFLSP LVDPNSEGGK GTSKAGAKPT WQKKKEANDP | 198) |
| | TWEQDYEKWK KRREEDPTAS VITTLEEYGI RPIFPLYTNT VTDIAWLPLQ | |
| | SNQFVRTWDR DMLQQAIERL LSWESWNKRV QEEYAKLKEK MAQLNEQLEG | |
| | GQEWISLLEQ YEENRERELR ENMTAANDKY RITKRQMKGW NELYELWSTF | |
| | PASASHEQYK EALKRVQQRL RGRFGDAHFF QYLMEEKNRL IWKGNPQRIH | |
| | YFVARNELTK RLEEAKQSAT MTLPNARKHP LWVRFDARGG NLQDYYLTAE | |
| | ADKPRSRRFV TFSQLIWPSE SGWMEKKDVE VELALSRQFY QQVKLLKNDK | |
| | GKQKIEFKDK GSGSTFNGHL GGAKLQLERG DLEKEEKNFE DGEIGSVYLN | |
| | VVIDFEPLQE VKNGRVQAPY GQVLQLIRRP NEFPKVTTYK SEQLVEWIKA | |
| | SPQHSAGVES LASGFRVMSI DLGLRAAAAT SIFSVEESSD KNAADFSYWI | |
| | EGTPLVAVHQ RSYMLRLPGE QVEKQVMEKR DERFQLHQRV KFQIRVLAQI | |
| | MRMANKQYGD RWDELDSLKQ AVEQKKSPLD QTDRTFWEGI VCDLTKVLPR | |
| | NEADWEQAVV QIHRKAEEYV GKAVQAWRKR FAADERKGIA GLSMWNIEEL | |
| | EGLRKLLISW SRRTRNPQEV NRFERGHTSH QRLLTHIQNV KEDRLKQLSH | |
| | AIVMTALGYV YDERKQEWCA EYPACQVILF ENLSQYRSNL DRSTKENSTL | |
| | MKWAHRSIPK YVHMQAEPYG IQIGDVRAEY SSRFYAKTGT PGIRCKKVRG | |
| | QDLQGRRFEN LQKRLVNEQF LTEEQVKQLR PGDIVPDDSG ELFMTLTDGS | |
| | GSKEVVFLQA DINAAHNLQK RFWQRYNELF KVSCRVIVRD EEEYLVPKTK | |
| | SVQAKLGKGL FVKKSDTAWK DVYVWDSQAK LKGKTTFTEE SESPEQLEDF | |
| | QEIIEEAEEA KGTYRTLFRD PSGVFFPESV WYPQKDFWGE VKRKLYGKLR | |
| | ERFLTKAR | |
| Alicyclobacillus | MAVKSIKVKL RLDDMPEIRA GLWKLHKEVN AGVRYYTEWL SLLRQENLYR | (SEQ |
| acidoterrestris | RSPNGDGEQE CDKTAEECKA ELLERLRARQ VENGHRGPAG SDDELLQLAR | ID NO: |
| WP_021296342.1 | QLYELLVPQA IGAKGDAQQI ARKFLSPLAD KDAVGGLGIA KAGNKPRWVR | 199) |
| | MREAGEPGWE EEKEKAETRK SADRTADVLR ALADFGLKPL MRVYTDSEMS | |
| | SVEWKPLRKG QAVRTWDRDM FQQAIERMMS WESWNQRVGQ | |
| | EYAKLVEQKN | |
| | RFEQKNFVGQ EHLVHLVNQL QQDMKEASPG LESKEQTAHY VTGRALRGSD | |
| | KVFEKWGKLA PDAPFDLYDA EIKNVQRRNT RRFGSHDLFA KLAEPEYQAL | |
| | WREDASFLTR YAVYNSILRK LNHAKMFATF TLPDATAHPI WTRFDKLGGN | |
| | LHQYTFLFNE FGERRHAIRF HKLLKVENGV AREVDDVTVP ISMSEQLDNL | |
| | LPRDPNEPIA LYFRDYGAEQ HFTGEFGGAK IQCRRDQLAH MHRRRGARDV | |
| | YLNVSVRVQS QSEARGERRP PYAAVFRLVG DNHRAFVHFD KLSDYLAEHP | |
| | DDGKLGSEGL LSGLRVMSVD LGLRTSASIS VFRVARKDEL KPNSKGRVPF | |
| | FFPIKGNDNL VAVHERSQLL KLPGETESKD LRAIREERQR TLRQLRTQLA | |
| | YLRLLVRCGS EDVGRRERSW AKLIEQPVDA ANHMTPDWRE AFENELQKLK | |
| | SLHGICSDKE WMDAVYESVR RVWRHMGKQV RDWRKDVRSG ERPKIRGYAK | |
| | DVVGGNSIEQ IEYLERQYKF LKSWSFFGKV SGQVIRAEKG SRFAITLREH | |
| | IDHAKEDRLK KLADRIIMEA LGYVYALDER GKGKWVAKYP PCQLILLEEL | |
| | SEYQFNNDRP PSENNQLMQW SHRGVFQELI NQAQVHDLLV GTMYAAFSSR | |
| | FDARTGAPGI RCRRVPARCT QEHNPEPFPW WLNKFVVEHT LDACPLRADD | |
| | LIPTGEGEIF VSPFSAEEGD FHQIHADLNA AQNLQQRLWS DFDISQIRLR | |
| | CDWGEVDGEL VLIPRLTGKR TADSYSNKVF YTNTGVTYYE RERGKKRRKV | |
| | FAQEKLSEEE AELLVEADEA REKSVVLMRD PSGIINRGNW TRQKEFWSMV | |
| | NQRIEGYLVK QIRSRVPLQD SACENTGDI | |
| Alicyclobacillus | MTVRSIRVKL AVGSPQYRDV RRGLWKTHEI MNQGVRYYCE WLVLMRQEPI | (SEQ |
| hesperidum | YDEDEHGLTV VQRTREDIQA ELLSRLRTLQ SAHQHSGDMG TDEELLSLMR | ID NO: |
| WP_074693942.1 | QLYEQLVPSS VDKNKSGDAR MIARNFFNPL TNPNSQGGLG ISNAGRKPKW | 200) |
| | LLKKLSGDPT WEEDYKKAME QKQESSVSFL LLELRRFGLH PIFLPYTDTV | |
| | LEVSWAPKKA RQWVRKWDYD LFQQSIERML SWESWTRRVK ERFEKLVESE | |
| | KKFYDENFAT DPEFIKLAET LEGELQASSQ GFVAVDEHAF QIRPRSMRGF | |
| | DRVADEWCKL ADDAPIEEYE AAIKRVQARL GRNFGSYVLF AHLAKPEYWS | |
| | LWRSDPTKIL RFARLRALQR AVARAKRHAR LTLPDAIHHP IWIRYDAKGK | |
| | NIYSYRLLIP EKRSKRYYVE FSSLIMPDGE NRWAEHRNIR VPLAFSRQWE | |
| | RLHFSIMEDG SLCVQYRDPG VDEPLRAELG GAKIQFDRRY LIRRSSTLSA | |
| | GECGPVYLNV SVDVNPAHRP DVQVLQSAKL VSVSRDTNRI YLRPENLSAY | |
| | WKSQGDGTLP LRVMSVDLGV RSSAAVVICR LEHRDSVVSS GRRTATIYRI | |
| | AGTDEFVAVQ ERAFLLRLPG EGKGTNEDAP LRDVYAQLGT IRQGIQILRS | |
| | LLRLCDTKTP DERQEALHGL AQSLEPSGAW KDELHPHLVM LQGVVHDSVD | |
| | NWKQKVISVH RQMERILGHA VREWKVARKN AGKPPIRRGA GGLSLRRIRQ | |
| | LEQERRTLVA WSNHAREPGQ VVRIKRGTQV AQWLVERVNH LKEDRLKKLA | |
| | DLLIMTALGY VYDETKPSGH KWDKRYPPCQ IILMEDLSRY RFQSDRPPSE | |
| | NSQLMAWSHR RLLEILKLQA DLHKLIVGTV FPAFSSRFDA QSGAPGVRCR | |
| | SVKKQDIENA AQGKGWLARE LQRLNWTLEW LQPNDLIPTG DGELFVTPAC | |
| | CDRQKGIKIV HADLNAAQNL QRRFWGGHAE SLCRVTCDVV ERDGRRYAVP | |
| | RISNAFADSF YKVFGQGVFV STDEEDVYRW MVGEKISSRG RSRGRTSDEE | |
| | AEAETWIDEA REQQGKVIAL FRDASGQIHG GDWLVAKVFW GWVERLVTAR | |
| | LLSRMSEREA AAHKE | |
| Alicyclobacillus | MAVKSMKVKL RLDNMPEIRA GLWKLHTEVN AGVRYYTEWL SLLRQENLYR | (SEQ |
| acidiphilus | RSPNGDGEQE CYKTAEECKA ELLERLRARQ VENGHCGPAG SDDELLQLAR | ID NO: |
| WP_067623834.1 | QLYELLVPQA IGAKGDAQQI ARKFLSPLAD KDAVGGLGIA KAGNKPRWVR | 201) |
| | MREAGEPGWE EEKAKAEARK STDRTADVLR ALADFGLKPL MRVYTDSDMS | |
| | SVQWKPLRKG QAVRTWDRDM FQQAIERMMS WESWNQRVGE | |
| | AYAKLVEQKS | |
| | RFEQKNFVGQ EHLVQLVNQL QQDMKEASHG LESKEQTAHY LTGRALRGSD | |
| | KVFEKWEKLD PDAPFDLYDT EIKNVQRRNT RRFGSHDLFA KLAEPKYQAL | |
| | WREDASFLTR YAVYNSIVRK LNHAKMFATF TLPDATAHPI WTRFDKLGGN | |
| | LHQYTFLFNE FGEGRHAIRF QKLLTVEDGV AKEVDDVTVP ISMSAQLDDL | |
| | LPRDPHELVA LYFQDYGAEQ HLAGEFGGAK IQYRRDQLNH LHARRGARDV | |
| | YLNLSVRVQS QSEARGERRP PYAAVFRLVG DNHRAFVHFD KLSDYLAEHP | |
| | DDGKLGSEGL LSGLRVMSVD LGLRTSASIS VFRVARKDEL KPNSEGRVPF | |
| | CFPIEGNENL VAVHERSQLL KLPGETESKD LRAIREERQR TLRQLRTQLA | |
| | YLRLLVRCGS EDVGRRERSW AKLIEQPMDA NQMTPDWREA FEDELQKLKS | |
| | LYGICGDREW TEAVYESVRR VWRHMGKQVR DWRKDVRSGE RPKIRGYQKD | |
| | WVGGNSIEQI EYLERQYKFL KSWSFFGKVS GQVIRAEKGS RFAITLREHI | |
| | DHAKEDRLKK LADRIIMEAL GYVYALDDER GKGKWVAKYP PCQLILLEEL | |
| | SEYQFNNDRP PSENNQLMQW SHRGVFQELL NQAQVHDLLV GTMYAAFSSR | |
| | FDARTGAPGI RCRRVPARCA REQNPEPFPW WLNKFVAEHK LDGCPLRADD | |
| | LIPTGEGEFF VSPFSAEEGD FHQIHADLNA AQNLQRRLWS DFDISQIRLR | |
| | CDWGEVDGEP VLIPRTTGKR TADSYGNKVF YTKTGVTYYE RERGKKRRKV | |
| | FAQEELSEEE AELLVEADEA REKSVVLMRD PSGIINRGDW TRQKEFWSMV | |
| | NQRIEGYLVK QIRSRVRLQE SACENTGDI | |
| Alicyclobacillus | MAVKSIKVKL MLGHLPEIRE GLWHLHEAVN LGVRYYTEWL ALLRQGNLYR | (SEQ |
| macrosporangiidus | RGKDGAQECY MTAEQCRQEL LVRLRDRQKR NGHTGDPGTD EELLGVARRL | ID NO: |
| SFU30094.1 | YELLVPQSVG KKGQAQMLAS GFLSPLADPK SEGGKGTSKS GRKPAWMGMK | 202) |
| | EAGDSRWVEA KARYEANKAK DPTKQVIASL EMYGLRPLFD VFTETYKTIR | |
| | WMPLGKHQGV RAWDRDMFQQ SLERLMSWES WNERVGAEFA | |
| | RLVDRRDRFR | |
| | EKHFTGQEHL VALAQRLEQE MKEASPGFES KSSQAHRITK RALRGADGII | |
| | DDWLKLSEGE PVDRFDEILR KRQAQNPRRF GSHDLFLKLA EPVFQPLWRE | |
| | DPSFLSRWAS YNEVLNKLED AKQFATFTLP SPCSNPVWAR FENAEGTNIF | |
| | KYDFLFDHFG KGRHGVRFQR MIVMRDGVPT EVEGIVVPIA PSRQLDALAP | |
| | NDAASPIDVF VGDPAAPGAF RGQFGGAKIQ YRRSALVRKG RREEKAYLCG | |
| | FRLPSQRRTG TPADDAGEVF LNLSLRVESQ SEQAGRRNPP YAAVFHISDQ | |
| | TRRVIVRYGE IERYLAEHPD TGIPGSRGLT SGLRVMSVDL GLRTSAAISV | |
| | FRVAHRDELT PDAHGRQPFF FPIHGMDHLV ALHERSHLIR LPGETESKKV | |
| | RSIREQRLDR LNRLRSQMAS LRLLVRTGVL DEQKRDRNWE RLQSSMERGG | |
| | ERMPSDWWDL FQAQVRYLAQ HRDASGEAWG RMVQAAVRTL | |
| | WRQLAKQVRD | |
| | WRKEVRRNAD KVKIRGIARD VPGGHSLAQL DYLERQYRFL RSWSAFSVQA | |
| | GQWVRAERDS RFAVALREHI DNGKKDRLKK LADRILMEAL GYVYVTDGRR | |
| | AGQWQAVYPP CQLVLLEELS EYRFSNDRPP SENSQLMVWS HRGVLEELIH | |
| | QAQVHDVLVG TIPAAFSSRF DARTGAPGIR CRRVPSIPLK DAPSIPIWLS | |
| | HYLKQTERDA AALRPGELIP TGDGEFLVTP AGRGASGVRV VHADINAAHN | |
| | LQRRLWENFD LSDIRVRCDR REGKDGTVVL IPRLTNQRVK ERYSGVIFTS | |
| | EDGVSFTVGD AKTRRRSSAS QGEGDDLSDE EQELLAEADD ARERSVVLFR | |
| | DPSGFVNGGR WTAQRAFWGM VHNRIETLLA ERFSVSGAAE KVRG | |
| Sulfobacillus | RQSREDASPQ IIISASDLKA DLLYHARQQQ KEHVPRITGS DAEVLGALRQ | (SEQ |
| thermo- | VYELIVPSSV GKSGDSKTIA RKFLSPLTDP DSAGGRDQSA SGRKPTWTKM | ID NO: |
| sulfidooxidans | KAEGNPLWEE KFRQWKDRKD NDPTPFVLNQ LADYGLLPLI RLFTDVGENI | 203) |
| PSR34340.1 | FDPKKPGQFV RPWDRSMFQQ AIERLMSWES WNQRVRQEWE | |
| | ALTQKHSAFY | |
| | REQFTAEPDA ALYRVAQSLE EEMRKEHQGF ATDAPEAFRI RRVALKGFDR | |
| | LLERWQKTLG KNGQSATLLD DIRRVQSDLG DKFGSAPLYQ KLVDERWQRL | |
| | WTVDPTFLQR YAAFNDLTQR LQRAKRVANL TLPDAVAHPI WSRYEGPNAS | |
| | SGNRYHIHLP TTGQPSSVTF DRILWPDGDG GWYERKRVTV FLRPSHQVDR | |
| | IREAPTDSWV DNFPLVVEDQ SARTILRASW GGAKLEYDRN RLPRQLKKGV | |
| | PDSIYLSLTL NLDTTKPSGL FHMQQNGRVW IRKDVVMQYY NEIPGDNVQF | |
| | KPLYVMSVDL GIRSAAAVSI FSVQLKTGIE EHRLTYPVAD CPGLVAVHER | |
| | SVLLTMPGER REQRDRRYEQ QRQGLRELRT DMRGMNDLLR GAYVDGDRRE | |
| | EFLARLSKLE ETSPELWEPV YRSLNDSKMA PAAEWERLVV YCHRQVEQSL | |
| | SSRIQNLRSG RSAYRMSGGL SLDHVQDLER IRGIIASWTN HPRIPGSVVR | |
| | WQQGRSHTVA LGRHILELKR DRVKKVANYL IMTALGYAYD SKRARGEKWV | |
| | RRYPSCHLMV FEDLTRYRFR TDRPRSENRQ LMRWTHQELI AVTGIQAEPH | |
| | GILVGTMYAG FSSRFDAVTK APGVRGATVR QILRTRGMVR LKEIAADVGV | |
| | DINTLRPHDV LPTGDGEYLL SVVRHRDSYR LKQVHADINA AHNLQRRLWT | |
| | QDEVFRVSCR LALNSERVVA TPPPSYNKRY GKGFFEKGDN GVYIWKTGGK | |
| | IKISDMLEED MDIPEDTAEL LRGNSVTLFR DPSGTIAGGN WLEAKEFWGR | |
| | VNSLVNKGVR DKILGGIPVD NSSAHAE | |
| Spirochaeta sp. | MGLLLPSLSR TVNVTIHLIL HPRKKGSRHR EYAVMLDHAV RKIFLAHNWI | (SEQ |
| LUC14_002_19_ | KRAEAERQKF EADLYKIDRV PQEARDWLDE FCRERTESTG SIDGYHIRRK | ID NO: |
| P3 | AVLGWEALVE AWDQKDCLSV EDRIAAARDL QDNPGMDKFG DIWLYEALAS | 204) |
| OQX29950.1 | APCVWQKDGE PNAQILLDYV DAGEAEYKRS HYKVPAYRHP DPLLHPIFCD | |
| | FGQSRWSISF DIHEFKKNGE KNPVNIHALT MGLVSKKRIV KTELKWSSKR | |
| | LNSNLALSLE SPEDAIEVSR ATRLGRAAVG ASQDRAVNIA GLFESAGWNG | |
| | RLQAPRKQLE ALAKLEEDKS AEALAKALRN RIKWFITFSP KLQPHGPWME | |
| | YAERFSGEAP SRAAVIKGKY TVIHQDKTRR RPLAKLHLCR MPGLRVLSVD | |
| | LGHRHAAACA VWETLSSESM EKKCREAGCL PPAPEDLYLH LKKKNKTAVY | |
| | RRIGGNFLPD GNEHPAPWAK LDRQFIIDLQ GEEGCTRMAL AGEIWQVHCM | |
| | EKVFGRSIPL VDRLVRAGWG EKNKQPEILQ ELKQKGWVPL EVSKTNTGYH | |
| | YSLCVDSLMT LAVNTVRFAL RRHACRARIA YYMEGGAIPE GGLPENSGNK | |
| | DFIVEALMLW YELATDSRWN GSWEANFWDE NFDKKLAEIQ DAVNEREGDK | |
| | AKIIKQKERK ELLKKEFIPL AEGLLENSRR ISIASQWRMV WNEEDAIWQS | |
| | ELRSLRDWIL PKGTRGKKRT IRHVGGLSLS RLAVIKSLYR VQKSFYTRMK | |
| | PEGEPMDGTM AVGEGFGQKI LDDLETMKEQ RVKQLASRVV EAALGTGRIK | |
| | KPENNKTPKR PFTAVDEPCH AVVIENLTHY RPENKRTRRE NRQLMTWSSS | |
| | KVKKYLFESC QLHGLYLFEV QASYTSRQDS RTGAPGVRCS ELSVKKFLES | |
| | PFRQREIAHA EENMAQENPC NRYLIALHNK WKNREYDKTA PPLRIPHWGG | |
| | EIFVSALTGN TLQADLNAAA NIGLQALLDP DWPGRWWYVP AVKGCDGRRI | |
| | PHSKCSGAAC LDNWRVGLKN NLYTGVRTPL PGKNKGSTSG EDVHKSNAVE | |
| | KSTINLWRDI SVLPLTEGQW | |
| Bacillus hisashii | MATRSFILKI EPNEEVKKGL WKTHEVLNHG IAYYMNILKL IRQEAIYEHH | (SEQ |
| strain C4 v4 | EQDPKNPKKV SKAEIQAELW DFVLKMQKCN SFTHEVDKDE VFNILRELYE | ID NO: |
| mutant of | ELVPSSVEKK GEANQLSNKF LYPLVDPNSQ SGKGTASSGR KPRWYNLKIA | 205) |
| WP_095142515.1 | GDPSWEEEKK KWEEDKKKDP LAKILGKLAE YGLIPLFIPY TDSNEPIVKE | |
| K846R | IKWMEKSRNQ SVRRLDKDMF IQALERFLSW ESWNLKVKEE YEKVEKEYKT | |
| S893R | LEERIKEDIQ ALKALEQYEK ERQEQLLRDT LNTNEYRLSK RGLRGWREII | |
| E837G | QKWLKMDENE PSEKYLEVFK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDFPKVVNF KPKELTEWIK | |
| | DSKGKKLKSG IESLEIGLRV MSIDLGQRQA AAASIFEVVD QKPDIEGKLF | |
| | FPIKGTELYA VHRASFNIKL PGETLVKSRE VLRKAREDNL KLMNQKLNFL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYGERS RFENSRLMKW | |
| | SRREIPRQVA LQGEIYGLQV GEVGAQFSSR FHAKTGSPGI RCRVVTKEKL | |
| | QDNRFFKNLQ REGRLTLDKI AVLKEGDLYP DKGGEKFISL SKDRKCVTTH | |
| | ADINAAQNLQ KRFWTRTHGF YKVYCKAYQV DGQTVYIPES KDQKQKIIEE | |
| | FGEGYFILKD GVYEWVNAGK LKIKKGSSKQ SSSELVDSDI LKDSFDLASE | |
| | LKGEKLMLYR DPSGNVFPSD KWMAAGVFFG KLERILISKL TNQYSISTIE | |
| | DDSSKQSM | |

TABLE 7
Cas12c (C2c3) orthologs

| | | |
|---|---|---|
| OspCas12c | MTKLRHRQKK LTHDWAGSKK REVLGSNGKL QNPLLMPVKK GQVTEFRKAF | (SEQ ID |
| AWU30132.1 | SAYARATKGE MTDGRKNMFT HSFEPFKTKP SLHQCELADK AYQSLHSYLP | NO: 206) |
| KZX85786.1 | GSLAHFLLSA HALGFRIFSK SGEATAFQAS SKIEAYESKL ASELACVDLS | |
| | IQNLTISTLF NALTTSVRGK GEETSADPLI ARFYTLLTGK PLSRDTQGPE | |
| | RDLAEVISRK IASSFGTWKE MTANPLQSLQ FFEEELHALD ANVSLSPAFD | |
| | VLIKMNDLQG DLKNRTIVFD PDAPVFEYNA EDPADIIIKL TARYAKEAVI | |
| | KNQNVGNYVK NAITTTNANG LGWLLNKGLS LLPVSTDDEL LEFIGVERSH | |
| | PSCHALIELI AQLEAPELFE KNVFSDTRSE VQGMIDSAVS NHIARLSSSR | |
| | NSLSMDSEEL ERLIKSFQIH TPHCSLFIGA QSLSQQLESL PEALQSGVNS | |
| | ADILLGSTQY MLTNSLVEES IATYQRTLNR INYLSGVAGQ INGAIKRKAI | |
| | DGEKIHLPAA WSELISLPFI GQPVIDVESD LAHLKNQYQT LSNEFDTLIS | |
| | ALQKNFDLNF NKALLNRTQH FEAMCRSTKK NALSKPEIVS YRDLLARLTS | |
| | CLYRGSLVLR RAGIEVLKKH KIFESNSELR EHVHERKHFV FVSPLDRKAK | |
| | KLLRLTDSRP DLLHVIDEIL QHDNLENKDR ESLWLVRSGY LLAGLPDQLS | |
| | SSFINLPIIT QKGDRRLIDL IQYDQINRDA FVMLVTSAFK SNLSGLQYRA | |
| | NKQSFVVTRT LSPYLGSKLV YVPKDKDWLV PSQMFEGRFA DILQSDYMVW | |
| | KDAGRLCVID TAKHLSNIKK SVFSSEEVLA FLRELPHRTF IQTEVRGLGV | |
| | NVDGIAFNNG DIPSLKTFSN CVQVKVSRTN TSLVQTLNRW FEGGKVSPPS | |
| | IQFERAYYKK DDQIHEDAAK RKIRFQMPAT ELVHASDDAG WTPSYLLGID | |
| | PGEYGMGLSL VSINNGEVLD SGFIHINSLI NFASKKSNHQ TKVVPRQQYK | |
| | SPYANYLEQS KDSAAGDIAH ILDRLIYKLN ALPVFEALSG NSQSAADQVW | |
| | TKVLSFYTWG DNDAQNSIRK QHWFGASHWD IKGMLRQPPT EKKPKPYIAF | |
| | PGSQVSSYGN SQRCSCCGRN PIEQLREMAK DTSIKELKIR NSEIQLFDGT | |
| | IKLFNPDPST VIERRRHNLG PSRIPVADRT FKNISPSSLE FKELITIVSR | |
| | SIRHSPEFIA KKRGIGSEYF CAYSDCNSSL NSEANAAANV AQKFQKQLFF | |
| | EL | |
| QFN42172.1 | MRSNYHGGRN ARQWRKQISG LARRTKETVF TYKFPLETDA AEIDFDKAVQ | (SEQ ID |
| | TYGIAEGVGH GSLIGLVCAF HLSGFRLFSK AGEAMAFRNR SRYPTDAFAE | NO: 207) |
| | KLSAIMGIQL PTLSPEGLDL IFQSPPRSRD GIAPVWSENE VRNRLYTNWT | |
| | GRGPANKPDE HLLEIAGEIA KQVFPKFGGW DDLASDPDKA LAAADKYFQS | |
| | QGDFPSIASL PAAIMLSPAN STVDFEGDYI AIDPAAETLL HQAVSRCAAR | |
| | LGRERPDLDQ NKGPFVSSLQ DALVSSQNNG LSWLFGVGFQ HWKEKSPKEL | |
| | IDEYKVPADQ HGAVTQVKSF VDAIPLNPLF DTTHYGEFRA SVAGKVRSWV | |
| | ANYWKRLLDL KSLLATTEFT LPESISDPKA VSLFSGLLVD PQGLKKVADS | |
| | LPARLVSAEE AIDRLMGVGI PTAADIAQVE RVADEIGAFI GQVQQFNNQV | |
| | KQKLENLQDA DDEEFLKGLK IELPSGDKEP PAINRISGGA PDAAAEISEL | |
| | EEKLQRLLDA RSEHFQTISE WAEENAVTLD PIAAMVELER LRLAERGATG | |
| | DPEEYALRLL LQRIGRLANR VSPVSAGSIR ELLKPVFMEE REFNLFFHNR | |
| | LGSLYRSPYS TSRHQPFSID VGKAKAIDWI AGLDQISSDI EKALSGAGEA | |
| | LGDQLRDWIN LAGFAISQRL RGLPDTVPNA LAQVRCPDDV RIPPLLAMLL | |
| | EEDDIARDVC LKAFNLYVSA INGCLFGALR EGFIVRTRFQ RIGTDQIHYV | |
| | PKDKAWEYPD RLNTAKGPIN AAVSSDWIEK DGAVIKPVET VRNLSSTGFA | |
| | GAGVSEYLVQ APHDWYTPLD LRDVAHLVTG LPVEKNITKL KRLTNRTAFR | |
| | MVGASSFKTH LDSVLLSDKI KLGDFTIIID QHYRQSVTYG GKVKISYEPE | |
| | RLQVEAAVPV VDTRDRTVPE PDTLFDHIVA IDLGERSVGF AVFDIKSCLR | |
| | TGEVKPIHDN NGNPVVGTVA VPSIRRLMKA VRSHRRRRQP NQKVNQTYST | |
| | ALQNYRENVI GDVCNRIDTL MERYNAFPVL EFQIKNFQAG AKQLEIVYGS | |
| QFN42158.1 | MKKFELKQNF RNNYSGKTLR NFRQTLAQIA NKKSSDSILT IKFKLDCSKT | (SEQ ID |
| | GKLPKYENLI SLYDTIEDIK KGTLSYYLFT LIVSGFKFFG SASQAKAFST | NO: 208) |
| | KDIFKDNDFY NQFKIQSHLD LPDFVPSKIY QRLKKNVRST NGKDNAFKAS | |
| | VIVAEYRKEI GKLKNKDESS EHQCEELFKK IGTALETRFS SWQDLINNCS | |
| | TGCEIIDEIL NDSFGTLPSI KKMVLASTTQ SSDGEQDGIA IAYDPDSTFI | |
| | KSDELLNPYF AVATILKSMP PEIQQDKKSA YVKANLTTPT HNALSWIFGK | |
| | GLTLFQTEST EKLCAMFNVS DKRVIEQVQD AAKAVKLPAE LDLNHCTLKF | |
| | QDFRSSLGGH LDSWTTNYLK RLDELNDLLL NLPKNLSLPD IFMIDGKDFI | |
| | EYSGCNRDEI QQMIDFVVNE QNRIKLQESL NALLGKGNNQ ICSDDISTVK | |
| | DFSEIVNSLH SFVQQIDNSL EQSSNEANSI FSELKKKIEK NEKWDIWKNN | |
| | LKKIPKLNKL SGGVPDAWKE IREIEQKFHE ISENQKKHFT EVMEWIDAGN | |
| | GTIDIFESRF KYDELLKKSK KNNLQSADEL AFRSVLNKLG RFARQGNDLV | |
| | CEKIKNWFKE QNIFDSSKDF NRYFINQKGF IFKHPSSKKD NSPYNLSANL | |
| | LEKRYEVTNT VGALLEQCES DPAIVNDPFS MRSLVEFRAL WFSINISGIS | |
| | KEQHIPTKIA QPKLDDSTYQ ESVSPTLKYR LEKEQITSSE LNSIFTVYKS | |
| | LLSGLSIRLS RNSFYLRTKF SWIGNNSLIY CPKETTWKIP AAYFKSDLWN | |
| | EYKDKQILIV NEEYDVDVVK TFESVYKIVK SKDNNEKNRI LPLLKQLPHD | |
| | WMFKLPFGAS NAEKCKVLKL EKNNKKFKPL SVSKDSLARL SGPSTYFNQI | |
| | DEIMMNDESE LSEMTLLADE PVRQQMSNGK IEIIPDDYVM SLAIPITRSL | |
| | KKGNTESFPF KNIVSIDQGE AGFAYAVFKL SDCGNERAEP IATGLIPIPS | |
| | IRRLIHSVKK YRGKKQRIQN FNQKFDSTMF TLRENVTGDI CGLIVALMKK | |
| | YNAFPILEKQ VGNLESGSKQ LMLVYKAVNS KFLAAKVDMQ NDQRRSWWYQ | |
| | GNSWNTPILR ISNPNQSNNK NIVKNINGKK YEELKIYPGY SVSAYMTSCI | |
| | CHVCGRNALE LLKNDDSTGK VKKYQINQDG EVTIGGEVIK LYRKPDRLTP | |
| | VKNLAKKGNR ERTYASINER APMSKDTTQS RYFCVFKNCP CHNKEQHADV | |
| | NAAINIGRRF LKDCILDDNK EKD | |
| QFN42173.1 | MNARDWRKHV GVLAQQHKET TRTYTFPLDT TGSAIDFDAA LQAYNAVEGV | (SEQ ID |
| | GYGSLLGLAC AVHLSGFRLF STGKEAATFR NRARYPNAAF QAALRKELGT | NO: 209) |
| | TITTLTPETL DRLFSSRPKR RNGVPLPWNQ DSIRDRLYTN WVKPRPGDTP | |
| | DAVLFQIATG IAQEITEDVS SWTDLAKNSD RGLKAAHRYF ARVGGFPAFD | |
| | NLTPPATVQP TDTTIDYDPN APFHLVSHAD QTLIHQSISL CAHRIRQEDP | |
| | ALDPNKSGFI KQLQNNFLSQ TFYGLSWLFG AGYVHFRECT ANDLAIQYGI | |
| | PNNCRDGIHQ IKSFADAILP NTFFEKKHYR KDSRSVGKKA KSWISNYWQR | |
| | LLQLQTWVDD HTWVTLPQEL TEAQFKPLFR GLLVDAVELM AIAERLPQRL | |
| | ADCRDSLDCL MGKGPQAATK NDVEIVEKVR EEIESFVGQI EQLGNQLRHQ | |
| | LENENNDQVH RDNLHQLKNR LPLDLRRPQA LNKISGGVPD VAKSIRGLET | |
| | QLDQVLKERR SHFGRLTKWA KECGITLDPL QPLIESEKQR VAERGSAHDA | |
| | KELAIRLLLQ RIGRLGHRLS PTNATAIQEL LRPVFAVKRE FNLFFHNHMG | |
| | ALYRSPYSTS RHQPFQINVD VAHGTDWIGT IETLIQNLFT QIQDDALLRD | |
| | LVQLEGFVFS HKLRALPGVI PSELARPNNL QQMGLPALLL VLLQADQVHR | |
| | ETVLRVFNLY GSAINGYLFQ ALRPGFIVRA GFQRLETKKL RYVPKAQSWQ | |
| | YPDRLHHAKS AIKNSLSAGW IKKNHQGAIL PQKTLTALVK QKSLKDTGVP | |
| | EYLVQAPHDW YVPIDLRGPA IPIEGLTVGT EGPELTQLGP MKDDCAFRAI | |
| | GPSSFKSKID AGLLPQDVKY GDMTLIFDQH YQQSISFANG TFSIQYQPTS | |
| | LQVKAAIPWV DKRPRDTRNN SHLYDRIVAI DLGERKIGYA IFDLKQVLKS | |
| | EQLEPMREDG KPLIGSISIR SIRGLMKAVQ THRNRRQPNY RIDQTYSKAL | |
| | MHYRESVIGD VCNAIDTLCA RYGGFPVLES SVRNFEVGSA QLKTVYGSVS | |
| | RRYTWSAVDA HKNQRQQYWL GGTKDKIPIW THPYLMTREW DEKNSKWSNR | |
| | SKPLKMHPGV EVHPAGTSQI CHQCKRNPIG ALWNVADTVV LDDQGQLDLD | |
| | DGTIRLNSGY IDTTEIKRAR RKKIRLPENK PLTGSHKTSH VRAVARRNLR | |
| | QPPKSTRAKD TTQSRYTCLY VDCGHECHAD ENAAINIGRK YLQERIHIEA | |
| | SRQALSTR | |
| QFN42174.1 | MVAGLKKIKR DGVTMKSNYH GGVKARAWRK RIGGLARRQK ETVFTYKFPL | (SEQ ID |
| | ETEEAGIDFD KAVQTYGIAE GISQGSLIGL VCAFHLSGFR LFSKADETKA | NO: 210) |
| | FCNQGRYPNQ AFAEKLRNEL SVTLPKLSPQ SLDVLFQSSP KSKNGVAPEW | |
| | SKNAIRNRLY TNWTGKGAGT NPDEHLLEIA EDIAAEIDSD LDGWKDLEEH | |
| | PEKGLSAADR YFQAQGDFPS LTGLPPSVPL TPQNSTVAFE GDPVCLNPSD | |
| | NTLLHQAVAR CAGRILQEQP NLSPDKNRFI NQLQDELVSS QNNGLSWLFG | |
| | VGFKYWKEMS VDQLADDYKV KSTDLDALKQ VKSFIDAIPL NPLFDTPHYG | |
| | EFRASVAGKM RSWVKNYWKR LLDLKSQLGT ANINLPEGLD EQRAENLFSG | |
| | LLIDSKGLRQ VTDKLPSRLK KAEDTIDRLM GDGNPTSDDI EQVETVAAEI | |
| | SAFIGQVEQF NNQLEQRLEN PLEGDDETFL KQLKIDLPAE FKKPPAINRI | |
| | SGGSPDPTAE IAELEEKLDR LMSARKEHYE TIAEWASANK VTLDPMEAMT | |
| | TLEAQRLTER GAEGDQEEFA LRLLLQRIGR LANRLSPQGA TAIRDLLRPV | |
| | FTEKREFNLF FHNRMGSLYR SPYSTSRHQP FTIDVAVAKN TDWMDALDGI | |
| | AETIMKGLSQ AGDELSLRQL EEDEVSREVC LKAFNLYVSA INGCLFRALR | |
| | EGFIVRTKFQ RLERDVLSYV PKTKLWNYPQ RLDTARGPIH SALAAAWINK | |
| | EGSVIDPVET VTALSDTGFS DDGIPEYLVQ APHDWYLRDW INISGFSLSQ | |
| | RLRGLPDTVP GELALVRSAD DVRIPPMLAL TPIDLRDISK PVSGLPVKKN | |
| | ITGLKRQKKQ TAFRMVGPSS FKSHLDSTLL SEEVKLGDFT LIFDQYYKQR | |
| | VSYNGRVKIT FEPDRLHVEA AVPVIDKRVR PSTEEDALFD HLLAIDLGEK | |
| | RVGYAVYDIK ACLRTGDIKP LEDGDGKPIV GSVAVPSIRR LMKAVRSHRQ | |
| | QRQPNQKVNQ TYSTALMNYR ENVIGDVCNR IDTLMEKYNA FPVLESSVMN | |
| | FEAGSRQLEM VYGSVLHRYT YSKIDAHTAK RKEYWYTGEY WDHPYLMAHK | |
| | WNERTRSYSG SLSALTLYPG VMVHPAGTSQ RCHQCKRNPM VEIKQLTGQV | |
| | EINADGSLEL DDGTICLYEG YDYSPEEYKK AKREKRRLDP NVPLSGRHQA | |
| | KHVSAVAKRN LRRPTVSMMS GDTTQARYVC LYTDCDFTGH ADENAAINIG | |
| | WKYLTERIAL SESKDKAGV | |

TABLE 8
Cas12e (CasY) orthologs

| | | |
|---|---|---|
| APG80656.1 | MSKRHPRISG VKGYRLHAQR LEYTGKSGAM | (SEQ ID |
| GI: | RTIKYPLYSS PSGGRTVPRE IVSAINDDYV | NO: 211) |
| 1110962136 | GLYGLSNFDD LYNAEKRNEE KVYSVLDFWY | |
| QFN42175.1 | DCVQYGAVFS YTAPGLLKNV AEVRGGSYEL | |
| | TKTLKGSHLY DELQIDKVIK FLNKKEISRA | |
| | NGSLDKLKKD IIDCFKAEYR ERHKDQCNKL | |
| | ADDIKNAKKD AGASLGERQK KLFRDFFGIS | |
| | EQSENDKPSF TNPLNLTCCL LPFDTVNNNR | |
| | NRGEVLENKL KEYAQKLDKN EGSLEMWEYI | |
| | GIGNSGTAFS NFLGEGFLGR LRENKITELK | |
| | KAMMDITDAW RGQEQEEELE KRLRILAALT | |
| | IKLREPKFDN HWGGYRSDIN GKLSSWLQNY | |
| | INQTVKIKED LKGHKKDLKK AKEMINRFGE | |
| | SDTKEEAVVS SLLESIEKIV PDDSADDEKP | |
| | DIPAIAIYRR FLSDGRLTLN RFVQREDVQE | |
| | ALIKERLEAE KKKKPKKRKK KSDAEDEKET | |
| | IDFKELFPHL AKPLKLVPNF YGDSKRELYK | |
| | KYKNAAIYTD ALWKAVEKIY KSAFSSSLKN | |
| | SFFDTDFDKD FFIKRLQKIF SVYRRFNTDK | |
| | WKPIVKNSFA PYCDIVSLAE NEVLYKPKQS | |
| | RSRKSAAIDK NRVRLPSTEN IAKAGIALAR | |
| | ELSVAGFDWK DLLKKEEHEE YIDLIELHKT | |
| | ALALLLAVTE TQLDISALDF VENGTVKDFM | |
| | KTRDGNLVLE GRFLEMFSQS IVFSELRGLA | |
| | GLMSRKEFIT RSAIQTMNGK QAELLYIPHE | |
| | FQSAKITTPK EMSRAFLDLA PAEFATSLEP | |
| | ESLSEKSLLK LKQMRYYPHY FGYELTRTGQ | |
| | GIDGGVAENA LRLEKSPVKK REIKCKQYKT | |
| | LGRGQNKIVL YVRSSYYQTQ FLEWFLHRPK | |
| | NVQTDVAVSG SFLIDEKKVK TRWNYDALTV | |
| | ALEPVSGSER VFVSQPFTIF PEKSAEEEGQ | |
| | RYLGIDIGEY GIAYTALEIT GDSAKILDQN | |
| | FISDPQLKTL REEVKGLKLD QRRGTFAMPS | |
| | TKIARIRESL VHSLRNRIHH LALKHKAKIV | |
| | YELEVSRFEE GKQKIKKVYA TLKKADVYSE | |
| | IDADKNLQTT VWGKLAVASE ISASYTSQFC | |
| | GACKKLWRAE MQVDETITTQ ELIGTVRVIK | |
| | GGTLIDAIKD FMRPPIFDEN DTPFPKYRDF | |
| | CDKHHISKKM RGNSCLFICP FCRANADADI | |
| | QASQTIALLR YVKEEKKVED YFERFRKLKN | |
| | IKVLGQMKKI | |

### 6.4. Protospacer Adjacent Motif

As used herein, the term “protospacer adjacent sequence” or “protospacer adjacent motif” or “PAM” refers to an approximately 2-6 base pair DNA sequence (or a 2-, 3—, 4-, 5-, 6-, 7-, 8-, 9-, 10-, 11-, 12-long nucleotide sequence) that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease may be modified to alter the PAM specificity of the nuclease such that the nuclease recognizes alternative PAM sequence.

For example, with reference to the canonical SpCas9 amino acid sequence, the PAM specificity can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities and some embodiments are therefore chosen based on the desired PAM recognition. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These examples are not meant to be limiting. It will be further appreciated that non-SpCas9s bind a variety of PAM sequences, which makes them useful to expand the range of sequences that can be targeted according to the invention. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference). Gasiunas used cell-free biochemical screens to identify protospacer adjacent motif (PAM) and guide RNA requirements of 79 Cas9 proteins. (Gasiunas et al., A catalogue of biochemically diverse CRISPR-Cas9 orthologs, Nature Communications 11:5512 doi.org/10.1038/s41467-020-19344-1) The authors described 7 classes of gRNA and 50 different PAM requirement.

Oh, Y. et al. describe linking reverse transcriptase to a Francisella novicida Cas9 [FnCas9(H969A)] nickase module. (Oh, Y. et al., Expansion of the prime editing modality with Cas9 from Francisella novicida, bioRxiv 2021.05.25.445577; doi.org/10.1101/2021.05.25.445577). By increasing the distance to the PAM, the FnCas9(H969A) nickase module expands the region of a reverse transcription template (RTT) following the primer binding site.

### 6.5. Prime Editors

“Prime editor fusion protein” describes a protein that is used in prime editing. Prime editing uses CRISPR enzyme that nicks or cuts only single strand of double stranded DNA, i.e., a nickase; and a nickase can occur either naturally or by mutation or modification of a nuclease that makes double stranded cuts. Such an enzyme can be a catalytically-impaired Cas9 endonuclease (a nickase). Such an enzyme can be a Cas12a/b, MAD7, or variant thereof. The nickase is fused to an engineered reverse transcriptase (RT). The nickase is programmed (directed) with a prime-editing guide RNA (pegRNA). The skilled person in the art would appreciate that the pegRNA both specifies the target site and encodes the desired edit. Advantageously the nickase is a catalytically-impaired Cas9 endonuclease, a Cas9 nickase, that is fused to the reverse transcriptase. During genetic editing, the Cas9 nickase part of the protein is guided to the DNA target site by the pegRNA, whereby a nick or single stranded cut occurs. The reverse transcriptase domain then uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Afterward, optionally, the prime editor (PE) guides resolution of the heteroduplex to favor copying the edit onto the unedited strand, completing the process (typically achieved with a nickase gRNA).

As used herein, “PE1” refers to a PE complex comprising a fusion protein comprising Cas9(H840A) and a wild type MMLV RT having the following N-terminus to C-terminus structure: [NLS]-[Cas9(H840A)]-[linker]-[MMLV_RT(wt)]+a desired atgRNA (or PEgRNA). In various embodiments, the prime editors disclosed herein is comprised of PE1.

As used herein, “PE2” refers to a PE complex comprising a fusion protein comprising Cas9(H840A) and a variant MMLV RT having the following N-terminus to C-terminus structure:

[NLS]-[Cas9(H840A)]-[linker]-[MMLV_RT(D200N)(T330P)(L603W)(T306K)(W313F)]+a desired atgRNA (or PEgRNA). In various embodiments, the prime editors disclosed herein are comprised of PE2. In various embodiments, the prime editors disclosed herein is comprised of PE2 and co-expression of MMR protein MLH1dn, that is PE4.

As used herein, “PE3” refers to PE2 plus a second-strand nicking guide RNA that complexes with the PE2 and introduces a nick in the non-edited DNA strand. The induction of the second nick increases the chances of the unedited strand, rather than the edited strand, to be repaired. In various embodiments, the prime editors disclosed herein are comprised of PE3. In various embodiments, the prime editors disclosed herein are comprised of PE3 and co-expression of MMR protein MLH1dn, that is PE5.

As used herein, “PE3b” refers to PE3 but wherein the second-strand nicking guide RNA is designed for temporal control such that the second strand nick is not introduced until after the installation of the desired edit. This is achieved by designing a gRNA with a spacer sequence with mismatches to the unedited original allele that matches only the edited strand. Using this strategy, mismatches between the protospacer and the unedited allele should disfavor nicking by the sgRNA until after the editing event on the PAM strand takes place.

### 6.6. Guides for Prime Editing

Anzalone et al., 2019 (Nature 576:149) describes prime editing and a prime editing complex using a type II CRISPR and can be used herein. A prime editing complex consists of a type II CRISPR PE protein containing an RNA-guided DNA-nicking domain fused to a reverse transcriptase (RT) domain and complexed with a pegRNA. The pegRNA comprises (5′ to 3′) a spacer that is complementary to the target sequence of a genomic DNA, a nickase (e.g. Cas9) binding site, a reverse transcriptase template including editing positions, and primer binding site (PBS). The PE-pegRNA complex binds the target DNA and the CRISPR protein nicks the PAM-containing strand. The resulting 3′ end of the nicked target hybridizes to the primer-binding site (PBS) of the pegRNA, then primes reverse transcription of new DNA containing the desired edit using the RT template of the pegRNA. The overall structure of the pegRNA is like that of a typical type II sgRNA with a reverse transcriptase template/primer binding site appended to the 3′ end. The structure leaves the PBS at the 3′ end of the pegRNA free to bind to the nicked strand complementary to the target which forms the primer for reverse transcription.

Guide RNAs of CRISPRs differ in overall structure. For example, while the spacer of a type II gRNA is located at the 5′ end, the spacer of a type V gRNA is located towards the 3′ end, with the CRISPR protein (e.g. Cas12a) binding region located toward the 5′ end. Accordingly, the regions of a type V pegRNA are rearranged compared to a type II pegRNA. The overall structure of the pegRNA is like that of a typical type II sgRNA with a reverse transcriptase template/primer binding site appended to the 3′ end. The pegRNA comprises (5′ to 3′) a CRISPR protein-binding region, a spacer which is complementary to the target sequence of a genomic DNA, a reverse transcriptase template including editing positions, and primer binding site (PBS).

In typical embodiments, an atgRNA comprises a reverse transcriptase template that encodes, partially or in its entirety, an integration recognition site (also referred to as an integration target recognition site) or a recombinase recognition site (also referred to as a recombinase target recognition site). The integration target recognition site, which is to be placed at a desired location in the genome or intracellular nucleic acid, is referred to as a “beacon” site or an “attachment site” or a “landing pad” or “landing site.” An integration target recognition site or recombinase target recognition site incorporated into the pegRNA is referred to as an attachment site containing guide RNA (atgRNA).

### 6.7. Attachment Site-Containing Guide RNA (atgRNA)

As used herein, the term “attachment site-containing guide RNA” (atgRNA) and the like refer to an extended single guide RNA (sgRNA) comprising a primer binding site (PBS), a reverse transcriptase (RT) template sequence, and wherein the RT template encodes for an integration recognition site or a recombinase recognition site that can be recognized by a recombinase, integrase, or transposase. In some embodiments, the RT template comprises a clamp sequence and an integration recognition site. As referred to herein an atgRNA may be referred to as a guide RNA. An integration recognition site or recombinase target recognition site incorporated into the pegRNA is referred to as an attachment site containing guide RNA (atgRNA).

As used herein, the term “cognate integration recognition site” or “integration cognate” or “cognate pair” refers to a first integration recognition site (e.g., any of the integration recognition sites described herein) and a second integration recognition site (e.g., any of the integration recognition sites described herein) that can be recombined. Recombination between a first integration recognition site (e.g., any of the integration recognition sites described herein) and a second recognition site (e.g., any of the integration recognition sites described herein) is mediated by functional symmetry between the two integration recognition sites and the central dinucleotide of each of the two integration recognition sites. In some cases, a first integration recognition site (e.g., any of the integration recognition sites described herein) that can be recombined with a second integration recognition site (e.g., any of the integration recognition sites described herein) are referred to as a “cognate pair.” A non-limiting example of a cognate pair include an attB site and an attP site, whereby a serine integrase mediates recombination between the attB site and the attP site.

In typical embodiments, an atgRNA comprises a reverse transcriptase template that encodes, partially or in its entirety, an integration recognition site (also referred to as an integration target recognition site) or a recombinase recognition site (also referred to as a recombinase target recognition site). The integration target recognition site, which is to be placed at a desired location in the genome or intracellular nucleic acid, is referred to as a “beacon,” a “beacon” site or an “attachment site” or a “landing pad” or “landing site.” An integration target recognition site or recombinase target recognition site incorporated into the pegRNA is referred to as an attachment site containing guide RNA (atgRNA).

During genome editing, the primer binding site allows the 3′ end of the nicked DNA strand to hybridize to the atgRNA, while the RT template serves as a template for the synthesis of edited genetic information. The atgRNA is capable for instance, without limitation, of (i) identifying the target nucleotide sequence to be edited and (ii) encoding new genetic information that replaces (or in some cases adds) the targeted sequence. In some embodiments, the atgRNA is capable of (i) identifying the target nucleotide sequence to be edited and (ii) encoding an integration site that replaces (or inserts/deletes within) the targeted sequences.

In some embodiments, the co-delivery system described herein includes a polynucleotide sequence encoding an attachment site-containing guide RNA (atgRNA) packaged in an LNP. In some embodiments, the co-delivery system described herein includes a vector comprising a polynucleotide sequence encoding an atgRNA. In some embodiments, the atgRNA comprises a domain that is capable of guiding the prime editor fusion protein to a target sequence, thereby identifying the target nucleotide sequence to be edited; and a reverse transcriptase (RT) template that comprises a first integration recognition site. In some embodiments, the atgRNA comprises a domain that is capable of guiding the prime editor fusion protein (or prime editor system) to a target sequence, thereby identifying the target nucleotide sequence to be edited; and a reverse transcriptase (RT) template that comprises at least a portion first integration recognition site.

In some embodiments, the co-delivery system described herein includes a polynucleotide sequence encoding a first attachment site-containing guide RNA (atgRNA) and a polynucleotide nucleotide sequence encoding a second attachment site-containing guide RNA (atgRNA) packaged into the same LNP. In some embodiments, the co-delivery system described herein includes a polynucleotide sequence encoding a first attachment site-containing guide RNA (atgRNA) packaged into a first LNP and a polynucleotide nucleotide sequence encoding a second attachment site-containing guide RNA (atgRNA) packaged into a second LNP.

In some embodiments, the co-delivery system described herein includes a vector comprising a polynucleotide sequence encoding a first attachment site-containing guide RNA (atgRNA), a polynucleotide sequence encoding a second atgRNA, or both.

In some embodiments, the co-delivery system described herein includes a polynucleotide sequence encoding a first attachment site-containing guide RNA (atgRNA) packaged into a first LNP and a vector comprising a polynucleotide sequence encoding a second atgRNA.

In some embodiments, where the co-delivery system contains a first atgRNA and a second atgRNA, the first atgRNA and the second atgRNA are an at least first pair of atgRNAs, where the at least first pair of atgRNAs have domains that are capable of guiding the gene editor protein or prime editor fusion protein to a target sequence, the first atgRNA further includes a first RT template that comprises at least a portion of the first integration recognition site; and the second atgRNA further includes a second RT template that comprises at least a portion of the first integration recognition site, and the first atgRNA and the second atgRNAs collectively encode the entirety of the first integration recognition site.

In some embodiments, the first atgRNA's reverse transcriptase template encodes for a first single-stranded DNA sequence (i.e., a first DNA flap) that contains a complementary region to a second single-stranded DNA sequence (i.e., a second DNA flap) encoded by a second atgRNA comprising a second reverse transcriptase template. In certain embodiments, the complementary region between the first and second single-stranded DNA sequences is comprised of more than 5 consecutive bases of an integrase target recognition site. In certain embodiments, the complementary region between the first and second single-stranded DNA sequences is comprised of more than 10 consecutive bases of an integrase target recognition site. In certain embodiments, the complementary region between the first and second single-stranded DNA sequences is comprised of more than 20 consecutive bases of an integrase target recognition site. In certain embodiments, the complementary region between the first and second single-stranded DNA sequences is comprised of more than 30 consecutive bases of an integrase target recognition site. Use of two guide RNAs that are (or encode DNA that is) partially complementarity to each other and comprised of consecutive bases of an integrase target recognition site are referred to as dual, paired, annealing, complementary, or twin attachment site-containing guide RNAs (atgRNAs). In certain embodiments, use of two guide RNAs that are (or encode DNA that is) full complementarity to each other and comprised of consecutive bases of an integrase target recognition site are referred to as dual, paired, annealing, complementary, or twin attachment site-containing guide RNAs (atgRNAs).

In some embodiments, upon introducing the nucleic acid construct into a cell, the first atgRNA incorporates the first integration recognition site into the cell's genome at the target sequence.

Table 9 includes atgRNAs, sgRNAs and nicking guides that can be used herein. Spacers are labeled in capital font (SPACER), RT regions in bold capital (RT REGION), AttB sites in bold lower case (attB site), and PBS in capital italics (PBS). Unless otherwise denoted, the AttB is for Bxb1.

TABLE 9

| | | SEQ ID |
|---|---|---|
| Description | Sequence (5′-3′) | NO |
| ACTB N-term | GCTATTCTCGCAGCTCACCAgttttagagctagaaatagcaagttaaaataaggctagtccgtt | 212 |
| PBS 13 RT | atcaacttgaaaaagtggcaccgagtcggtgcGACGAGCGCGGCGATATCATCATCCA | |
| 29 AttB 46 | TGGccggatgatcctgacgacggagaccgccgtcgtcgacaagccggcc | |
| atgRNA | TGAGCTGCGAGAA | |
| ACTB N-term | GCTATTCTCGCAGCTCACCAgtttgagagctatgctggaaacagcatagcaagttcaaataa | 213 |
| PBS 13 RT | ggctagtccgttatcaacttgaaaaagtggcaccgagtcggtgcGACGAGCGCGGCGATATC | |
| 29 AttB 46 | ATCATCCATGGccggatgatcctgacgacggagaccgccgtcgtcgacaagccggcc | |
| atgRNA with v2 | TGAGCTGCGA GAA | |
| scaffold | | |
| ACTB N-term | GCTATTCTCGCAGCTCACCAgttttagagctagaaatagcaagttaaaataaggctagtccgtt | 214 |
| PBS_13_RT_29 | atcaacttgaaaaagtggcaccgagtcggtgcGAGTCGGTGCGACGAGCGCGGCG | |
| with TP901-1 | ATATCATCATCCATGGcacaattaacatctcaatcaaggtaaa | |
| minimal AttB f | TGCTTGAGCTGCGAGAA | |
| atgRNA | | |
| ACTB N-term | GCTATTCTCGCAGCTCACCAgttttagagctagaaatagcaagttaaaataaggctagtccgtt | 215 |
| PBS_13_RT_29 | atcaacttgaaaaagtggcaccgagtcggtgcGAGTCGGTGCGACGAGCGCGGCG | |
| with TP901-1 | ATATCATCATCCATGGagcatttaccttgattgagatgttaattgtg | |
| minimal AttB rc | TGAGCTGCGAGAA | |
| atgRNA | | |
| ACTB N-term | GCTATTCTCGCAGCTCACCAgttttagagctagaaatagcaagttaaaataaggctagtccgtt | 216 |
| PBS_13_RT_29 | atcaacttgaaaaagtggcaccgagtcggtgcGAGTCGGTGCGACGAGCGCGGCG | |
| with PhiBT1 | ATATCATCATCCATGGcaggtttttgacgaaagtgatccagatgatccag | |
| minimal AttB f | TGAGCTGCGAGAA | |
| atgRNA | | |
| ACTB N-term | GCTATTCTCGCAGCTCACCAgttttagagctagaaatagcaagttaaaataaggctagtccgtt | 217 |
| PBS_13_RT_29 | atcaacttgaaaaagtggcaccgagtcggtgcGAGTCGGTGCGACGAGCGCGGCG | |
| with PhiBT1 | ATATCATCATCCATGGctggatcatctggatcactttcgtcaaaaacctg | |
| minimal AttB rc | TGAGCTGCGAGAA | |
| atgRNA | | |
| ACTB N-term | GAAGCCGGCCTTGCACATGCgttttagagctagaaatagcaagttaaaataaggctagtccg | 218 |
| Nicking guide 1 | ttat caacttgaaaaagtggcaccgagtcggtgc | |
| +48 guide | | |
| ACTB N-term | GAAGCCGGCCTTGCACATGCgttttagagctagaaatagcaagttaaaataaggctagtccg | 219 |
| PBS_18_RT_16 | ttatcaacttgaaaaagtggcaccgagtcggtgcATATCATCATCCATGGtaccgttcgtatag | |
| with_Lo x71_Cre | catacattatacgaagttat | |
| atgRNA | TGAGCTGCGAGAATAGCC | |
| ACTB N-term | GAAGCCGGCCTTGCACATGCgttttagagctagaaatagcaagttaaaataaggctagtccg | 220 |
| PBS_13_RT_29 | ttatcaacttgaaaaagtggcaccgagtcggtgcGACGAGCGCGGCGATATCATCATCC | |
| with_Lo x71_Cre | ATGGtaccgttcgtatagcatacattatacgaagttat | |
| atgRNA | TGAGCTGCGAGAA | |
| ACTB N-term | GCTATTCTCGCAGCTCACCAgttttagagctagaaatagcaagttaaaataaggctagtccgtt | 221 |
| PBS 13 RT | atcaacttgaaaaagtggcaccgagtcggtgcTCGACGACGAGCGCGGCGATATCAT | |
| 34 atgRNA | CATCCATGGccggatgatcctgacgacggagaccgccgtcgtcgacaagccggcc | |
| | TGAGCTGCGAGAA | |
| ACTB N-term | GCTATTCTCGCAGCTCACCAgttttagagctagaaatagcaagttaaaataaggctagtccgtt | 222 |
| PBS 13 RT | atcaacttgaaaaagtggcaccgagtcggtgcGAGCGCGGCGATATCATCATCCATGG | |
| 26 atgRNA | ccggatgatcctgacgacggagaccgccgtcgtcgacaagccggcc | |
| | TGAGCTGCGAGAA | |
| ACTB N-term | GCTATTCTCGCAGCTCACCAgttttagagctagaaatagcaagttaaaataaggctagtccgtt | 223 |
| PBS 13 RT | atcaacttgaaaaagtggcaccgagtcggtgcCGCGGCGATATCATCATCCATGGccgg | |
| 23 atgRNA | atgatcctgacgacggagaccgccgtcgtcgacaagccggccTGAGCTGCGAGAA | |
| ACTB N-term | GCTATTCTCGCAGCTCACCAgttttagagctagaaatagcaagttaaaataaggctagtccgtt | 224 |
| PBS 13 RT | atcaacttgaaaaagtggcaccgagtcggtgcGGCGATATCATCATCCATGGccggatga | |
| 20 atgRNA | tcctgacgacggagaccgccgtcgtcgacaagccggccTGAGCTGCGAGAA | |
| ACTB N-term | GCTATTCTCGCAGCTCACCAgttttagagctagaaatagcaagttaaaataaggctagtccgtt | 225 |
| PBS 13 RT | atcaacttgaaaaagtggcaccgagtcggtgcATATCATCATCCATGGccggatgatcctga | |
| 16 atgRNA | cgacggagaccgccgtcgtcgacaagccggccTGAGCTGCGAGAA | |
| ACTB N-term | GCTATTCTCGCAGCTCACCAgttttagagctagaaatagcaagttaaaataaggctagtccgtt | 226 |
| PBS 18 RT | atcaacttgaaaaagtggcaccgagtcggtgcTCGACGACGAGCGCGGCGATATCAT | |
| 34 atgRNA | CATCCATGGccggatgatcctgacgacggagaccgccgtcgtcgacaagccggcc | |
| | TGAGCTGCGAGAATAGCC | |
| ACTB N-term | GCTATTCTCGCAGCTCACCAgttttagagctagaaatagcaagttaaaataaggctagtccgtt | 227 |
| PBS 18 RT | atcaacttgaaaaagtggcaccgagtcggtgcGACGAGCGCGGCGATATCATCATCCA | |
| 29 atgRNA | TGGccggatgatcctgacgacggagaccgccgtcgtcgacaagccggcc | |
| | TGAGCTGCGAGAATAGCC | |
| ACTB N-term | GCTATTCTCGCAGCTCACCAgttttagagctagaaatagcaagttaaaataaggctagtccgtt | 228 |
| PBS 18 RT | atcaacttgaaaaagtggcaccgagtcggtgcATATCATCATCCATGGccggatgatcctga | |
| 16 atgRNA | cgacggagaccgccgtcgtcgacaagccggccTGAGCTGCGAGAATAGCC | |
| LMNB1 N-term | GCTGTCTCCGCCGCCCGCCAgttttagagctagaaatagcaagttaaaataaggctagtccg | 229 |
| PBS 13 RT 39 | ttatcaacttgaaaaagtggcaccgagtcggtgcCTGCCCATCCGCGGCGGCACGGGG | |
| atgRNA | GTCGCAGTCGCCATGccggatgatcctgacgacggagaccgccgtcgtcgacaagccg | |
| | gccCGGGCGGCGGAGA | |
| LMNB1 N-term | GCTGTCTCCGCCGCCCGCCAgttttagagctagaaatagcaagttaaaataaggctagtccg | 230 |
| PBS 13 RT 34 | ttatcaacttgaaaaagtggcaccgagtcggtgcCATCCGCGGCGGCACGGGGGTCGC | |
| atgRNA | AGTCGCCATGccggatgatcctgacgacggagaccgccgtcgtcgacaagccggccCG | |
| | GGCGGCGGAGA | |
| LMNB1 N-term | GCTGTCTCCGCCGCCCGCCAgttttagagctagaaatagcaagttaaaataaggctagtccg | 231 |
| PBS 13 RT 29 | ttatcaacttgaaaaagtggcaccgagtcggtgcGCGGCGGCACGGGGGTCGCAGTCG | |
| atgRNA | CCATGccggatgatcctgacgacggagaccgccgtcgtcgacaagccggccCGGGCGG | |
| | CGGAGA | |
| LMNB1 N-term | GCTGTCTCCGCCGCCCGCCAgttttagagctagaaatagcaagttaaaataaggctagtccg | 232 |
| PBS 13 RT 24 | ttatcaacttgaaaaagtggcaccgagtcggtgcGGCACGGGGGTCGCAGTCGCCATGc | |
| atgRNA | cggatgatcctgacgacggagaccgccgtcgtcgacaagccggccCGGGCGGCGGAGA | |
| LMNB1 N-term | GCTGTCTCCGCCGCCCGCCAgttttagagctagaaatagcaagttaaaataaggctagtccg | 233 |
| PBS 13 RT 19 | ttatcaacttgaaaaagtggcaccgagtcggtgcGGGGGTCGCAGTCGCCATGccggatg | |
| atgRNA | atcctgacgacggagaccgccgtcgtcgacaagccggccCGGGCGGCGGAGA | |
| LMNB1 N-term | GCTGTCTCCGCCGCCCGCCAgttttagagctagaaatagcaagttaaaataaggctagtccg | 234 |
| PBS 18 RT 39 | ttatcaacttgaaaaagtggcaccgagtcggtgcCTGCCCATCCGCGGCGGCACGGGG | |
| atgRNA | GTCGCAGTCGCCATGccggatgatcctgacgacggagaccgccgtcgtcgacaagccg | |
| | gccCGGGCGGCGGAGACAGCG | |
| LMNB1 N-term | GCTGTCTCCGCCGCCCGCCAgttttagagctagaaatagcaagttaaaataaggctagtccg | 235 |
| PBS 18 RT 34 | ttatcaacttgaaaaagtggcaccgagtcggtgcCATCCGCGGCGGCACGGGGGTCGC | |
| atgRNA | AGTCGCCATGccggatgatcctgacgacggagaccgccgtcgtcgacaagccggccCG | |
| | GGCGGCGGAGACAGCG | |
| LMNB1 N-term | GCTGTCTCCGCCGCCCGCCAgttttagagctagaaatagcaagttaaaataaggctagtccg | 236 |
| PBS 18 RT 29 | ttatcaacttgaaaaagtggcaccgagtcggtgcGCGGCGGCACGGGGGTCGCAGTCG | |
| atgRNA | CCATGccggatgatcctgacgacggagaccgccgtcgtcgacaagccggccCGGGCGG | |
| | CGGAGACAGCG | |
| LMNB1 N-term | GCTGTCTCCGCCGCCCGCCAgttttagagctagaaatagcaagttaaaataaggctagtccg | 237 |
| PBS 18 RT 24 | ttatcaacttgaaaaagtggcaccgagtcggtgcGGCACGGGGGTCGCAGTCGCCATGc | |
| atgRNA | cggatgatcctgacgacggagaccgccgtcgtcgacaagccggccCGGGCGGCGGAGA | |
| | CAGCG | |
| LMNB1 N-term | GCTGTCTCCGCCGCCCGCCAgttttagagctagaaatagcaagttaaaataaggctagtccg | 238 |
| PBS 18 RT 19 | ttatcaacttgaaaaagtggcaccgagtcggtgcGGGGGTCGCAGTCGCCATGccggatg | |
| atgRNA | atcctgacgacggagaccgccgtcgtcgacaagccggccCGGGCGGCGGAGACAGC | |
| | G | |
| LMNB1 N-term | GCGTGGTGGGGCCGCCAGCGgttttagagctagaaatagcaagttaaaataaggctagtcc | 239 |
| Nicking guide 1 | gttatcaacttgaaaaagtggcaccgagtcggtgc | |
| +46 | | |
| ACTB N-term | GCTATTCTCGCAGCTCACCAgttttagagctagaaatagcaagttaaaataaggctagtccgtt | 240 |
| PBS 13 RT | atcaacttgaaaaagtggcaccgagtcggtgcGACGAGCGCGGCGATATCATCATCCA | |
| 29 AttB 42 | TGGggatgatcctgacgacggagaccgccgtcgtcgacaagccgg | |
| atgRNA | TGAGCTGCGAGAA | |
| ACTB N-term | GCTATTCTCGCAGCTCACCAgttttagagctagaaatagcaagttaaaataaggctagtccgtt | 241 |
| PBS 13 RT | atcaacttgaaaaagtggcaccgagtcggtgcGACGAGCGCGGCGATATCATCATCCA | |
| 29 AttB 40 | TGGgatgatcctgacgacggagaccgccgtcgtcgacaagccgTGAGCTGCGAGAA | |
| atgRNA | | |
| ACTB N-term | GCTATTCTCGCAGCTCACCAgttttagagctagaaatagcaagttaaaataaggctagtccgtt | 242 |
| PBS 13 RT | atcaacttgaaaaagtggcaccgagtcggtgcGACGAGCGCGGCGATATCATCATCCA | |
| 29 AttB 38 | TGGatgatcctgacgacggagaccgccgtcgtcgacaagccTGAGCTGCGAGAA | |
| atgRNA | | |
| ACTB N-term | GCTATTCTCGCAGCTCACCAgttttagagctagaaatagcaagttaaaataaggctagtccgtt | 243 |
| PBS 13 RT | atcaacttgaaaaagtggcaccgagtcggtgcGACGAGCGCGGCGATATCATCATCCA | |
| 29 AttB 36 | TGGtgatcctgacgacggagaccgccgtcgtcgacaagc | |
| atgRNA | TGAGCTGCGAGAA | |
| LMNB1 N-term | GCTGTCTCCGCCGCCCGCCAgttttagagctagaaatagcaagttaaaataaggctagtccg | 244 |
| PBS 13 | ttatcaacttgaaaaagtggcaccgagtcggtgcGCGGCGGCACGGGGGTCGCAGTCG | |
| RT 29 AttB 44 | CCATGcggatgatcctgacgacggagaccgccgtcgtcgacaagccggcCGGGCGGC | |
| atgRNA v2 | GGAGA | |
| LMNB1 N-term | GCTGTCTCCGCCGCCCGCCAgttttagagctagaaatagcaagttaaaataaggctagtccg | 245 |
| PBS 13 | ttatcaacttgaaaaagtggcaccgagtcggtgcGCGGCGGCACGGGGGTCGCAGTCG | |
| RT 29 AttB 42 | CCATGggatgatcctgacgacggagaccgccgtcgtcgacaagccggCGGGCGGCGG | |
| atgRNA v2 | AGA | |
| LMNB1 N-term | GCTGTCTCCGCCGCCCGCCAgttttagagctagaaatagcaagttaaaataaggctagtccg | 246 |
| PBS 13 | ttatcaacttgaaaaagtggcaccgagtcggtgcGCGGCGGCACGGGGGTCGCAGTCG | |
| RT 29 AttB 40 | CCATGgatgatcctgacgacggagaccgccgtcgtcgacaagccgCGGGCGGCGGAG | |
| atgRNA v2 | A | |
| LMNB1 N-term | GCTGTCTCCGCCGCCCGCCAgttttagagctagaaatagcaagttaaaataaggctagtccg | 247 |
| PBS 13 | ttatcaacttgaaaaagtggcaccgagtcggtgcGCGGCGGCACGGGGGTCGCAGTCG | |
| RT 29 AttB 38 | CCATGatgatcctgacgacggagaccgccgtcgtcgacaagccCGGGCGGCGGAGA | |
| atgRNA v2 | | |
| NOLC1 N-term | GCGTATTGCCTGGAGGATGGgttttagagctagaaatagcaagttaaaataaggctagtccg | 248 |
| PBS 18 | ttatcaacttgaaaaagtggcaccgagtcggtgcGAACCACGCGGCGAATGCCGGCGT | |
| RT 29 AttB 46 | CCGCCccggatgatcctgacgacggagaccgccgtcgtcgacaagccggccTCCTCCA | |
| atgRNA | GGCAATACGCG | |
| NOLC1 N-term | GCGTATTGCCTGGAGGATGGgttttagagctagaaatagcaagttaaaataaggctagtccg | 249 |
| PBS 13 | ttatcaacttgaaaaagtggcaccgagtcggtgcGAACCACGCGGCGAATGCCGGCGT | |
| RT 29 AttB 46 | CCGCCccggatgatcctgacgacggagaccgccgtcgtcgacaagccggccTCCTCCA | |
| atgRNA | GGCAAT | |
| NOLC1 N-term | GCGTATTGCCTGGAGGATGGgttttagagctagaaatagcaagttaaaataaggctagtccg | 250 |
| PBS 13 | ttatcaacttgaaaaagtggcaccgagtcggtgcGAACCACGCGGCGAATGCCGGCGT | |
| RT 29 AttB 44 | CCGCCcggatgatcctgacgacggagaccgccgtcgtcgacaagccggcTCCTCCAGG | |
| atgRNA | CAAT | |
| NOLC1 N-term | GCGTATTGCCTGGAGGATGGgttttagagctagaaatagcaagttaaaataaggctagtccg | 251 |
| PBS 13 | ttatcaacttgaaaaagtggcaccgagtcggtgcGAACCACGCGGCGAATGCCGGCGT | |
| RT 29 AttB 42 | CCGCCggatgatcctgacgacggagaccgccgtcgtcgacaagccggTCCTCCAGGCA | |
| atgRNA | AT | |
| NOLC1 N-term | GCGTATTGCCTGGAGGATGGgttttagagctagaaatagcaagttaaaataaggctagtccg | 252 |
| PBS 13 | ttatcaacttgaaaaagtggcaccgagtcggtgcGAACCACGCGGCGAATGCCGGCGT | |
| RT 29 AttB 40 | CCGCCgatgatcctgacgacggagaccgccgtcgtcgacaagccgTCCTCCAGGCAA | |
| atgRNA | T | |
| NOLC1 N-term | GCGTATTGCCTGGAGGATGGgttttagagctagaaatagcaagttaaaataaggctagtccg | 253 |
| PBS 13 | ttatcaacttgaaaaagtggcaccgagtcggtgcGAACCACGCGGCGAATGCCGGCGT | |
| RT 29 AttB 38 | CCGCCatgatcctgacgacggagaccgccgtcgtcgacaagccTCCTCCAGGCAAT | |
| atgRNA | | |
| NOLC1 nicking | GAGCCGAGCACGAGGGGATACgttttagagctagaaatagcaagttaaaataaggctagtc | 254 |
| guide -43 | cgttatcaacttgaaaaagtggcaccgagtcggtgc | |
| ACTB N-term | GCTATTCTCGCAGCTCACCAgttttagagctagaaatagcaagttaaaataaggctagtccgtt | 255 |
| PBS 13 RT | atcaacttgaaaaagtggcaccgagtcggtgcGGCGATATCATCATCCATGGatgatcctg | |
| 20 AttB 38 | acgacggagaccgccgtcgtcgacaagccTGAGCTGCGAGAA | |
| atgRNA | | |
| ACTB N-term | GCTATTCTCGCAGCTCACCAgttttagagctagaaatagcaagttaaaataaggctagtccgtt | 256 |
| PBS 13 RT | atcaacttgaaaaagtggcaccgagtcggtgcTATCATCATCCATGGatgatcctgacgacg | |
| 15 AttB 38 | gagaccgccgtcgtcgacaagcc | |
| atgRNA | TGAGCTGCGAGAA | |
| ACTB N-term | GCTATTCTCGCAGCTCACCAgttttagagctagaaatagcaagttaaaataaggctagtccgtt | 257 |
| PBS 13 RT | atcaacttgaaaaagtggcaccgagtcggtgcTCATCCATGGatgatcctgacgacggagacc | |
| 10 AttB 38 | gccgtcgtcgacaagcc | |
| atgRNA | TGAGCTGCGAGAA | |
| ACTB N-term | GCTATTCTCGCAGCTCACCAgttttagagctagaaatagcaagttaaaataaggctagtccgtt | 258 |
| PBS 9 RT | atcaacttgaaaaagtggcaccgagtcggtgcGGCGATATCATCATCCATGGatgatcctg | |
| 20 AttB 38 | acgacggagaccgccgtcgtcgacaagcc | |
| atgRNA | TGAGCTGCG | |
| ACTB N-term | GCTATTCTCGCAGCTCACCAgttttagagctagaaatagcaagttaaaataaggctagtccgtt | 259 |
| PBS 9 RT | atcaacttgaaaaagtggcaccgagtcggtgcTATCATCATCCATGGatgatcctgacgacg | |
| 15 AttB 38 | gagaccgccgtcgtcgacaagcc | |
| atgRNA | TGAGCTGCG | |
| ACTB N-term | GCTATTCTCGCAGCTCACCAgttttagagctagaaatagcaagttaaaataaggctagtccgtt | 260 |
| PBS 9 RT | atcaacttgaaaaagtggcaccgagtcggtgcTCATCCATGGatgatcctgacgacggagacc | |
| 10 AttB 38 | gccgtcgtcgacaagcc | |
| atgRNA | TGAGCTGCG | |
| LMNB1 N-term | GCTGTCTCCGCCGCCCGCCAgttttagagctagaaatagcaagttaaaataaggctagtccg | 261 |
| PBS 13 | ttatcaacttgaaaaagtggcaccgagtcggtgcCGGGGGTCGCAGTCGCCATGatgatcc | |
| RT 20 AttB 38 | tgacgacggagaccgccgtcgtcgacaagccCGGGCGGCGGAGA | |
| atgRNA | | |
| LMNB1 N-term | GCTGTCTCCGCCGCCCGCCAgttttagagctagaaatagcaagttaaaataaggctagtccg | 262 |
| PBS 13 | ttatcaacttgaaaaagtggcaccgagtcggtgcGTCGCAGTCGCCATGatgatcctgacgac | |
| RT 15 AttB 38 | ggagaccgccgtcgtcgacaagccCGGGCGGCGGAGA | |
| atgRNA | | |
| LMNB1 N-term | GCTGTCTCCGCCGCCCGCCAgttttagagctagaaatagcaagttaaaataaggctagtccg | 263 |
| PBS 13 | ttatcaacttgaaaaagtggcaccgagtcggtgcAGTCGCCATGatgatcctgacgacggagac | |
| RT 10 AttB 38 | cgccgtcgtcgacaagccCGGGCGGCGGAGA | |
| atgRNA | | |
| LMNB1 N-term | GCTGTCTCCGCCGCCCGCCAgttttagagctagaaatagcaagttaaaataaggctagtccg | 264 |
| PBS 9 | ttatcaacttgaaaaagtggcaccgagtcggtgcCGGGGGTCGCAGTCGCCATGatgatcc | |
| RT 20 AttB 38 | tgacgacggagaccgccgtcgtcgacaagccCGGGCGGCG | |
| atgRNA | | |
| LMNB1 N-term | GCTGTCTCCGCCGCCCGCCAgttttagagctagaaatagcaagttaaaataaggctagtccg | 265 |
| PBS 9 | ttatcaacttgaaaaagtggcaccgagtcggtgcGTCGCAGTCGCCATGatgatcctgacgac | |
| RT 15 AttB 38 | ggagaccgccgtcgtcgacaagccCGGGCGGCG | |
| atgRNA | | |
| LMNB1 N-term | GCTGTCTCCGCCGCCCGCCAgttttagagctagaaatagcaagttaaaataaggctagtccg | 266 |
| PBS 9 | ttatcaacttgaaaaagtggcaccgagtcggtgcAGTCGCCATGatgatcctgacgacggagac | |
| RT 10 AttB 38 | cgccgtcgtcgacaagccCGGGCGGCG | |
| atgRNA | | |
| SUPT16H N-term | GAGAAGCGGCGTCCGGGGCTAgttttagagctagaaatagcaagttaaaataaggctagtc | 267 |
| PBS 13 | cgttatcaacttgaaaaagtggcaccgagtcggtgcTCTTTGTCCAGAGTCACAGCCATA | |
| RT 24 Bxb1- | ccggatgatcctgacgacggagaccgccgtcgtcgacaagccggccCCCCGGACGCCG | |
| GT_Initial length | C | |
| SRRM2 N-term | GGGCACGGGGCCATGTACAAgttttagagctagaaatagcaagttaaaataaggctagtccg | 268 |
| PBS 13 | ttatcaacttgaaaaagtggcaccgagtcggtgcGGCGTCGGCAGCCCGATCCCGTTGc | |
| RT 24 Bxb1 Initial | cggatgatcctgacgacggagaccgccgtcgtcgacaagccggccTACATGGCCCCGT | |
| length | | |
| DEPDC4 N-term | GTGTCAGGTGGGGCGGGGCTAgttttagagctagaaatagcaagttaaaataaggctagtc | 269 |
| PBS 18 | cgttatcaacttgaaaaagtggcaccgagtcggtgcGCTGGCTCCTCCCCTGGCACCATA | |
| RT 24 Bxb1 Initial | ccggatgatcctgacgacggagaccgccgtcgtcgacaagccggccCCCCGCCCCACC | |
| length | TGACAC | |
| NES N-term PBS | GAGTGGGTCAGACGAGCAGGAgttttagagctagaaatagcaagttaaaataaggctagtc | 270 |
| 13 RT | cgttatcaacttgaaaaagtggcaccgagtcggtgcCGACTCCTCCCCCATGCAGCCCTC | |
| 29 Bxb1 Initial | CATCccggatgatcctgacgacggagaccgccgtcgtcgacaagccggcc | |
| length | TGCTCGTCTGACC | |
| SUPT 16H nicking | GCAGCCACCCGCTCTCGGCCCgttttagagctagaaatagcaagttaaaataaggctagtc | 271 |
| guide −53 | cgttatcaacttgaaaaagtggcaccgagtcggtgc | |
| SRRM2 N-term | GTGTAGTCAGGCCGCTCACCCgttttagagctagaaatagcaagttaaaataaggctagtcc | 272 |
| nicking guide 1 | gttatcaacttgaaaaagtggcaccgagtcggtgc | |
| +87 | | |
| DEPDC4 N-term | GCTGACAAGTCTACGGAACCTgttttagagctagaaatagcaagttaaaataaggctagtcc | 273 |
| Nicking guide 1 | gttatcaacttgaaaaagtggcaccgagtcggtgc | |
| +59 | | |
| NES N-term | GCTCCTCCAGCGCCTTGACCgttttagagctagaaatagcaagttaaaataaggctagtccgt | 274 |
| Nicking guide 2 | tatcaacttgaaaaagtggcaccgagtcggtgc | |
| +79 | | |
| HITI_ACTB_ | GCTATTCTCGCAGCTCACCAgttttagagctagaaatagcaagttaaaataaggctagtccgtt | 275 |
| guide | atcaacttgaaaaagtggcaccgagtcggtgc | |
| HITI_SUPTH16_ | AGAAGCGGCGTCCGGGGCTAgttttagagctagaaatagcaagttaaaataaggctagtcc | 276 |
| guide | gttatcaacttgaaaaagtggcaccgagtcggtgc | |
| HITI_SRRM2_ | GGGCACGGGGCCATGTACAAgttttagagctagaaatagcaagttaaaataaggctagtccg | 277 |
| guide | ttatcaacttgaaaaagtggcaccgagtcggtgc | |
| HITI_NOLC1_ | GCGTATTGCCTGGAGGATGGgttttagagctagaaatagcaagttaaaataaggctagtccg | 278 |
| guide | ttatcaacttgaaaaagtggcaccgagtcggtgc | |
| HITI_DEPDC4_ | TGTCAGGTGGGGCGGGGCTAgttttagagctagaaatagcaagttaaaataaggctagtcc | 279 |
| guide | gttatcaacttgaaaaagtggcaccgagtcggtgc | |
| HITI_NES_ | AGTGGGTCAGACGAGCAGGAgttttagagctagaaatagcaagttaaaataaggctagtccg | 280 |
| guide | ttatcaacttgaaaaagtggcaccgagtcggtgc | |
| HITI_LMNB1_ | GCTGTCTCCGCCGCCCGCCAgttttagagctagaaatagcaagttaaaataaggctagtccg | 281 |
| guide | ttatcaacttgaaaaagtggcaccgagtcggtgc | |
| HDR Cas9 ACTB | GCTATTCTCGCAGCTCACCAgttttagagctagaaatagcaagttaaaataaggctagtccgtt | 275 |
| guide | atcaacttgaaaaagtggcaccgagtcggtgc | |
| HDR Cas9 | GGGGTCGCAGTCGCCATGGCgttttagagctagaaatagcaagttaaaataaggctagtcc | 282 |
| LMNB1 guide | gttatcaacttgaaaaagtggcaccgagtcggtgc | |
| ACTB N-term | GCTATTCTCGCAGCTCACCAgttttagagctagaaatagcaagttaaaataaggctagtccgtt | 283 |
| PBS 13 RT | atcaacttgaaaaagtggcaccgagtcggtgcGACGAGCGCGGCGATATCATCATCCA | |
| 29 AttB original | TGGccggatgatcctgacgacggagXXcgccgtcgtcgacaagccggccTGAGCTGCG | |
| length atgRNAs | AGAA | |
| for dinucleotides | XX: CG, GC, AT, TA, GG, TT, GA, AG, CC, TC, CT, AA, TG, GT, CA, AC | |
| ACTB N-term | GCTATTCTCGCAGCTCACCAgttttagagctagaaatagcaagttaaaataaggctagtccgtt | 284 |
| PBS 13 RT | atcaacttgaaaaagtggcaccgagtcggtgcGACGAGCGCGGCGATATCATCATCCA | |
| 29 atgRNA with | TGccggatgatcctgacgacggagACcgccgtcgtcgacaagccggccTGAGCTGCGA | |
| AttB 46 GT for | GAA | |
| fusion | | |
| ACTB N-term | GCTATTCTCGCAGCTCACCAgttttagagctagaaatagcaagttaaaataaggctagtccgtt | 285 |
| PBS 13 RT | atcaacttgaaaaagtggcaccgagtcggtgcGACGAGCGCGGCGATATCATCATCCA | |
| 29 atgRNA with | TGccggatgatcctgacgacggagAGcgccgtcgtcgacaagccggccTGAGCTGCGA | |
| AttB 46 CT for | GAA | |
| multiplexing | | |
| NOLC1 N-term | GCGTATTGCCTGGAGGATGGgttttagagctagaaatagcaagttaaaataaggctagtccg | 286 |
| PBS 18 | ttatcaacttgaaaaagtggcaccgagtcggtgcGAACCACGCGGCGAATGCCGGCGT | |
| RT 29 atgRNA | CCGCCccggatgatcctgacgacggagTCcgccgtcgtcgacaagccggccTCCTCCA | |
| with AttB 46 GA | GGCAATACGCG | |
| for multiplexing | | |
| LMNB1 N-term | GCTGTCTCCGCCGCCCGCCAgttttagagctagaaatagcaagttaaaataaggctagtccg | 287 |
| PBS 18 | ttatcaacttgaaaaagtggcaccgagtcggtgcGCGGCGGCACGGGGGTCGCAGTCG | |
| RT 29 atgRNA | CCATGccggatgatcctgacgacggagCTcgccgtcgtcgacaagccggccCGGGCGG | |
| with AttB 46 AG | CGGAGACAGCG | |
| for multiplexing | | |
| EMX1 Cas9 | GTCACCTCCAATGACTAGGGgttttagagctagaaatagcaagttaaaataaggctagtccgt | 288 |
| guide 1 | tatcaacttgaaaaagtggcaccgagtcggtgc | |
| EMX1 Cas9 | GGGCAACCACAAACCCACGAgttttagagctagaaatagcaagttaaaataaggctagtccg | 289 |
| guide 2 | ttatcaacttgaaaaagtggcaccgagtcggtgc | |
| ACTB N-term | GCTATTCTCGCAGCTCACCAgttttagagctagaaatagcaagttaaaataaggctagtccgtt | 290 |
| PBS 13 RT | atcaacttgaaaaagtggcaccgagtcggtgcGACGAGCGCGGCGATATCATCATCCA | |
| 29 AttB 56 GA | TGGctatgccggatgatcctgacgacggagtccgccgtcgtcgacaagccggccctagcTGA | |
| atgRNA | GCTGCGAGAA | |
| ACTB N-term | GCTATTCTCGCAGCTCACCAgttttagagctagaaatagcaagttaaaataaggctagtccgtt | 291 |
| PBS 13 RT | atcaacttgaaaaagtggcaccgagtcggtgcGACGAGCGCGGCGATATCATCATCCA | |
| 29 AttB 51 GA | TGGtgccggatgatcctgacgacggagtccgccgtcgtcgacaagccggccctaTGAGCT | |
| atgRNA | GCGAGAA | |
| ACTB N-term | GCTATTCTCGCAGCTCACCAgttttagagctagaaatagcaagttaaaataaggctagtccgtt | 292 |
| PBS 13 RT | atcaacttgaaaaagtggcaccgagtcggtgcGACGAGCGCGGCGATATCATCATCCA | |
| 29 AttB 46 GA | TGGccggatgatcctgacgacggagtccgccgtcgtcgacaagccggccTGAGCTGCGA | |
| atgRNA | GAA | |
| ACTB N-term | GCTATTCTCGCAGCTCACCAgttttagagctagaaatagcaagttaaaataaggctagtccgtt | 293 |
| PBS 13 RT | atcaacttgaaaaagtggcaccgagtcggtgcGACGAGCGCGGCGATATCATCATCCA | |
| 29 AttB 41 GA | TGGggatgatcctgacgacggagtccgccgtcgtcgacaagccgTGAGCTGCGAGAA | |
| atgRNA | | |
| ACTB N-term | GCTATTCTCGCAGCTCACCAgttttagagctagaaatagcaagttaaaataaggctagtccgtt | 294 |
| PBS 13 RT | atcaacttgaaaaagtggcaccgagtcggtgcGACGAGCGCGGCGATATCATCATCCA | |
| 29 AttB 36 GA | TGGtgatcctgacgacggagtccgccgtcgtcgacaagcTGAGCTGCGAGAA | |
| atgRNA | | |
| ACTB N-term | GCTATTCTCGCAGCTCACCAgttttagagctagaaatagcaagttaaaataaggctagtccgtt | 295 |
| PBS 13 RT | atcaacttgaaaaagtggcaccgagtcggtgcGACGAGCGCGGCGATATCATCATCCA | |
| 29 AttB 31 GA | TGGatcctgacgacggagtccgccgtcgtcgacaTGAGCTGCGAGAA | |
| atgRNA | | |
| ACTB N-term | GCTATTCTCGCAGCTCACCAgttttagagctagaaatagcaagttaaaataaggctagtccgtt | 296 |
| PBS 13 RT | atcaacttgaaaaagtggcaccgagtcggtgcGACGAGCGCGGCGATATCATCATCCA | |
| 29 AttB 26 GA | TGGcctgacgacggagtccgccgtcgtcgTGAGCTGCGAGAA | |
| atgRNA | | |
| ACTB N-term | GCTATTCTCGCAGCTCACCAgttttagagctagaaatagcaagttaaaataaggctagtccgtt | 297 |
| PBS 13 RT | atcaacttgaaaaagtggcaccgagtcggtgcGACGAGCGCGGCGATATCATCATCCA | |
| 29 AttB 21 GA | TGGtgacgacggagtccgccgtcgTGAGCTGCGAGAA | |
| atgRNA | | |
| ACTB N-term | GCTATTCTCGCAGCTCACCAgttttagagctagaaatagcaagttaaaataaggctagtccgtt | 298 |
| PBS 13 RT | atcaacttgaaaaagtggcaccgagtcggtgcGACGAGCGCGGCGATATCATCATCCA | |
| 29 AttB 16 GA | TGGacgacggagtccgccgTGAGCTGCGAGAA | |
| atgRNA | | |
| ACTB N-term | GCTATTCTCGCAGCTCACCAgttttagagctagaaatagcaagttaaaataaggctagtccgtt | 299 |
| PBS 13 RT | atcaacttgaaaaagtggcaccgagtcggtgcGACGAGCGCGGCGATATCATCATCCA | |
| 29 AttB 11 GA | TGGgacggagtccgTGAGCTGCGAGAA | |
| atgRNA | | |
| ACTB N-term | GCTATTCTCGCAGCTCACCAgttttagagctagaaatagcaagttaaaataaggctagtccgtt | 300 |
| PBS 13 RT | atcaacttgaaaaagtggcaccgagtcggtgcGACGAGCGCGGCGATATCATCATCCA | |
| 29 AttB 6 GA | TGGcggagtTGAGCTGCGAGAA | |
| atgRNA | | |
| ACTB N-term | GAAGCCGGCCTTGCACATGCgttttagagctagaaatagcaagttaaaataaggctagtccg | 301 |
| PBS_18_RT_34 | ttatcaacttgaaaaagtggcaccgagtcggtgcTCGACGACGAGCGCGGCGATATCAT | |
| with_Lo x71_Cre | CATCCATGGtaccgttcgtatagcatacattatacgaagttatTGAGCTGCGAGAATAG | |
| atgRNA | CC | |
| ACTB N-term | GAAGCCGGCCTTGCACATGCgttttagagctagaaatagcaagttaaaataaggctagtccg | 302 |
| PBS_18_RT_29 | ttatcaacttgaaaaagtggcaccgagtcggtgcGACGAGCGCGGCGATATCATCATCC | |
| with_Lo x71_Cre | ATGGtaccgttcgtatagcatacattatacgaagttatTGAGCTGCGAGAATAGCC | |
| atgRNA | | |
| ACTB N-term | GAAGCCGGCCTTGCACATGCgttttagagctagaaatagcaagttaaaataaggctagtccg | 303 |
| PBS_13_RT_34_ | ttatcaacttgaaaaagtggcaccgagtcggtgcTCGACGACGAGCGCGGCGATATCAT | |
| with_Lo x71_Cre | CATCCATGGtaccgttcgtatagcatacattatacgaagttatTGAGCTGCGAGAA | |
| atgRNA | | |
| ACTB N-term | GAAGCCGGCCTTGCACATGCgttttagagctagaaatagcaagttaaaataaggctagtccg | 304 |
| PBS_13_RT_16 | ttatcaacttgaaaaagtggcaccgagtcggtgcATATCATCATCCATGGtaccgttcgtatag | |
| with_Lo x71_Cre | catacattatacgaagttatTGAGCTGCGAGAA | |
| atgRNA | | |
| ACTB N-term | CCCCACGATGGAGGGGAAGAgttttagagctagaaatagcaagttaaaataaggctagtccg | 305 |
| Nicking guide 2 | ttatcaacttgaaaaagtggcaccgagtcggtgc | |
| +93 guide | | |
| LMNB1 N-term | CCTTCTCCTGGAGCCGCGACgttttagagctagaaatagcaagttaaaataaggctagtccgt | 306 |
| Nicking guide 2 | tatcaacttgaaaaagtggcaccgagtcggtgc | |
| +87 guide | | |
| ACTB N-term | GCTATTCTCGCAGCTCACCAgttttagagctagaaatagcaagttaaaataaggctagtccgtt | 307 |
| PBS 13 RT 29 | atcaacttgaaaaagtggcaccgagtcggtgcGACGAGCGCGGCGATATCATCATCCA | |
| AttB 46 | TGGcattatatgttcttacagtatggcggcccggattgtaaaaacatataatgTGAGCTGCGA | |
| N191352_143_72 | GAA | |
| integrase | | |
| ACTB N-term | GCTATTCTCGCAGCTCACCAgttttagagctagaaatagcaagttaaaataaggctagtccgtt | 308 |
| PBS 13 RT | atcaacttgaaaaagtggcaccgagtcggtgcGACGAGCGCGGCGATATCATCATCCA | |
| 29 AttB 46 | TGGcgttatagggtattacagtatggcggtcggtactgcaataccctataacgTGAGCTGCG | |
| N684346_90_69 | AGAA | |
| integrase | | |
| ACTB N-term | GCTATTCTCGCAGCTCACCAgttttagagctagaaatagcaagttaaaataaggctagtccgtt | 309 |
| PBS 13 RT 29 | atcaacttgaaaaagtggcaccgagtcggtgcGACGAGCGCGGCGATATCATCATCCA | |
| AttB 46 | TGGtgtatcattttcatatagttagcacctgcacactatatgaaaatgataca | |
| N675015_95_5 | TGAGCTGCGAGAA | |
| integrase | | |
| ACTB N-term | GCTATTCTCGCAGCTCACCAgttttagagctagaaatagcaagttaaaataaggctagtccgtt | 310 |
| PBS 13 RT 29 | atcaacttgaaaaagtggcaccgagtcggtgcGACGAGCGCGGCGATATCATCATCCA | |
| AttB 46 | TGGtgtctactatctgtatatgcgacacatgtggcataaagacatagtagacaTGAGCTGCG | |
| N189929_49_54 | AGAA | |
| integrase | | |
| ACTB N-term | GCTATTCTCGCAGCTCACCAgttttagagctagaaatagcaagttaaaataaggctagtccgtt | 311 |
| PBS 13 RT 29 | atcaacttgaaaaagtggcaccgagtcggtgcGACGAGCGCGGCGATATCATCATCCA | |
| AttB 46 | TGGcatcgaccctgacgcatgcggaggcggcgctccatgcgtctgacctcattTGAGCTGC | |
| N203911_45186 | GAGAA | |
| 6 | | |
| integrase | | |
| ACTB N-term | GCTATTCTCGCAGCTCACCAgttttagagctagaaatagcaagttaaaataaggctagtccgtt | 312 |
| PBS 13 RT 29 | atcaacttgaaaaagtggcaccgagtcggtgcGACGAGCGCGGCGATATCATCATCCA | |
| AttB 46 | TGGgttagtacccaaatgacaaaaggtcatccttttatcatttgggtactaacTGAGCTGCGA | |
| N687663_53_29 | GAA | |
| integrase | | |
| ACTB N-term | GCTATTCTCGCAGCTCACCAgttttagagctagaaatagcaagttaaaataaggctagtccgtt | 313 |
| PBS 13 RT 29 | atcaacttgaaaaagtggcaccgagtcggtgcGACGAGCGCGGCGATATCATCATCCA | |
| AttB 46 | TGGcttattaaaacccgttccgcttctgtcaaagcggcatcggttttataaacTGAGCTGCGA | |
| N687611_90_68 | GAA | |
| integrase | | |
| ACTB N-term | GCTATTCTCGCAGCTCACCAgttttagagctagaaatagcaagttaaaataaggctagtccgtt | 314 |
| PBS 13 RT 29 | atcaacttgaaaaagtggcaccgagtcggtgcGACGAGCGCGGCGATATCATCATCCA | |
| AttB 46 | TGGggcgtgatggtcgtgaacctcaacatgacgacgaacacgacctcgcggccTGAGCTG | |
| N190156_234_12 | CGAGAA | |
| integrase | | |
| ACTB N-term | GCTATTCTCGCAGCTCACCAgttttagagctagaaatagcaagttaaaataaggctagtccgtt | 315 |
| PBS 13 RT 29 | atcaacttgaaaaagtggcaccgagtcggtgcGACGAGCGCGGCGATATCATCATCCA | |
| AttB 46 | TGGtctacatcttgaatatatcaagttataactttgaattatatcagtttataTGAGCTGCGAGA | |
| N191533_224_76 | A | |
| integrase | | |
| ACTB N-term | GCTATTCTCGCAGCTCACCAgttttagagctagaaatagcaagttaaaataaggctagtccgtt | 316 |
| PBS 13 RT 29 | atcaacttgaaaaagtggcaccgagtcggtgcGACGAGCGCGGCGATATCATCATCCA | |
| AttB 46 | TGGaattatatctaaaagcactaagctccgccatactgcttttagatataataTGAGCTGCGA | |
| N208621_9_15 | GAA | |
| integrase | | |
| ACTB N-term | GCTATTCTCGCAGCTCACCAgttttagagctagaaatagcaagttaaaataaggctagtccgtt | 317 |
| PBS 13 RT 29 | atcaacttgaaaaagtggcaccgagtcggtgcGACGAGCGCGGCGATATCATCATCCA | |
| AttB 46 | TGGgatatggggaagtgaatcagtacaaccgccacagtaccTGAGCTGCGAGAA | |
| Bacillus_cereus | | |
| AH187_38 bp_Att | | |
| ACTB N-term | GCTATTCTCGCAGCTCACCAgttttagagctagaaatagcaagttaaaataaggctagtccgtt | 318 |
| PBS 13 RT 29 | atcaacttgaaaaagtggcaccgagtcggtgcGACGAGCGCGGCGATATCATCATCCA | |
| AttB 46 | TGGggtactgtggcggttgtactgattcacttccccatatcTGAGCTGCGAGAA | |
| Bacillus_cereus | | |
| AH187_38 | | |
| bp_Att_rc | | |
| ACTB N-term | GCTATTCTCGCAGCTCACCAgttttagagctagaaatagcaagttaaaataaggctagtccgtt | 319 |
| PBS 13 RT 29 | atcaacttgaaaaagtggcaccgagtcggtgcGACGAGCGCGGCGATATCATCATCCA | |
| AttB 46 | TGGtgggtggtacaggtgccacattagttgtaccatttatgTGAGCTGCGAGAA | |
| Staphylococcus_ | | |
| Iugdunensis | | |
| _N920143_38 bp_ | | |
| Att | | |
| ACTB N-term | GCTATTCTCGCAGCTCACCAgttttagagctagaaatagcaagttaaaataaggctagtccgtt | 320 |
| PBS 13 RT 29 | atcaacttgaaaaagtggcaccgagtcggtgcGACGAGCGCGGCGATATCATCATCCA | |
| AttB 46 | TGGcataaatggtacaactaatgtggcacctgtaccacccaTGAGCTGCGAGAA | |
| Staphylococcus_ | | |
| Iugdunensis | | |
| N920143_38 bp | | |
| Att_rc | | |
| ACTB N-term | GCTATTCTCGCAGCTCACCAgttttagagctagaaatagcaagttaaaataaggctagtccgtt | 321 |
| PBS 13 RT 29 | atcaacttgaaaaagtggcaccgagtcggtgcGACGAGCGCGGCGATATCATCATCCA | |
| AttB 46 | TGGgttgtttttccagatccagttggtcctgtaaatataagTGAGCTGCGAGAA | |
| Bacillus_cytotoxicus | | |
| _NVH_391- | | |
| 98_38 bp_Att | | |
| ACTB N-term | GCTATTCTCGCAGCTCACCAgttttagagctagaaatagcaagttaaaataaggctagtccgtt | 322 |
| PBS 13 RT 29 | atcaacttgaaaaagtggcaccgagtcggtgcGACGAGCGCGGCGATATCATCATCCA | |
| AttB 46 | TGGcttatatttacaggaccaactggatctggaaaaacaacTGAGCTGCGAGAA | |
| Bacillus_cytotoxicus | | |
| _NVH_391- | | |
| 98_38 bp_Att_rc | | |
| ACTB N-term | GCTATTCTCGCAGCTCACCAgttttagagctagaaatagcaagttaaaataaggctagtccgtt | 323 |
| PBS 13 RT 29 | atcaacttgaaaaagtggcaccgagtcggtgcGACGAGCGCGGCGATATCATCATCCA | |
| AttB 46 | TGGgtactgtggcggttgtactgattcacttccccatatTGAGCTGCGAGAA | |
| Bacillus_cereus | | |
| AH187_Att 36 bp | | |
| ACTB N-term | GCTATTCTCGCAGCTCACCAgttttagagctagaaatagcaagttaaaataaggctagtccgtt | 324 |
| PBS 13 RT 29 | atcaacttgaaaaagtggcaccgagtcggtgcGACGAGCGCGGCGATATCATCATCCA | |
| AttB 46 | TGGtactgtggcggttgtactgattcacttccccataTGAGCTGCGAGAA | |
| Bacillus_cereus | | |
| AH187_Att 34 bp | | |
| ACTB N-term | GCTATTCTCGCAGCTCACCAgttttagagctagaaatagcaagttaaaataaggctagtccgtt | 325 |
| PBS 13 RT 29 | atcaacttgaaaaagtggcaccgagtcggtgcGACGAGCGCGGCGATATCATCATCCA | |
| AttB 46 | TGGactgtggcggttgtactgattcacttccccatTGAGCTGCGAGAA | |
| Bacillus_cereus | | |
| AH187_Att 32 bp | | |
| ACTB N-term | GCTATTCTCGCAGCTCACCAgttttagagctagaaatagcaagttaaaataaggctagtccgtt | 326 |
| PBS 13 RT 29 | atcaacttgaaaaagtggcaccgagtcggtgcGACGAGCGCGGCGATATCATCATCCA | |
| AttB 46 | TGGatatggggaagtgaatcagtacaaccgccacagtacTGAGCTGCGAGAA | |
| Bacillus_cereus | | |
| AH187_Att | | |
| rc 36 bp | | |
| ACTB N-term | GCTATTCTCGCAGCTCACCAgttttagagctagaaatagcaagttaaaataaggctagtccgtt | 327 |
| PBS 13 RT 29 | atcaacttgaaaaagtggcaccgagtcggtgcGACGAGCGCGGCGATATCATCATCCA | |
| AttB 46 | TGGtatggggaagtgaatcagtacaaccgccacagtaTGAGCTGCGAGAA | |
| Bacillus_cereus | | |
| AH187_Att | | |
| _rc 34 bp | | |
| ACTB N-term | GCTATTCTCGCAGCTCACCAgttttagagctagaaatagcaagttaaaataaggctagtccgtt | 328 |
| PBS 13 RT 29 | atcaacttgaaaaagtggcaccgagtcggtgcGACGAGCGCGGCGATATCATCATCCA | |
| AttB 46 | TGGatggggaagtgaatcagtacaaccgccacagtTGAGCTGCGAGAA | |
| Bacillus_cereus | | |
| AH187_Att | | |
| _rc 32 bp | | |
| ACTB N-term | GCTATTCTCGCAGCTCACCAgttttagagctagaaatagcaagttaaaataaggctagtccgtt | 329 |
| PBS 13 RT 29 | atcaacttgaaaaagtggcaccgagtcggtgcGACGAGCGCGGCGATATCATCATCCA | |
| AttB 46 | TGGataaatggtacaactaatgtggcacctgtaccacccTGAGCTGCGAGAA | |
| Staphylococcus_ | | |
| Iugdunensis | | |
| N920143_Att | | |
| 36 bp | | |
| ACTB N-term | GCTATTCTCGCAGCTCACCAgttttagagctagaaatagcaagttaaaataaggctagtccgtt | 330 |
| PBS 13 RT 29 | atcaacttgaaaaagtggcaccgagtcggtgcGACGAGCGCGGCGATATCATCATCCA | |
| AttB 46 | TGGtaaatggtacaactaatgtggcacctgtaccaccTGAGCTGCGAGAA | |
| Staphylococcus_ | | |
| Iugdunensis | | |
| N920143_Att | | |
| 34 bp | | |
| ACTB N-term | GCTATTCTCGCAGCTCACCAgttttagagctagaaatagcaagttaaaataaggctagtccgtt | 331 |
| PBS 13 RT 29 | atcaacttgaaaaagtggcaccgagtcggtgcGACGAGCGCGGCGATATCATCATCCA | |
| AttB 46 | TGGaaatggtacaactaatgtggcacctgtaccacTGAGCTGCGAGAA | |
| Staphylococcus_ | | |
| Iugdunensis | | |
| N920143_Att | | |
| 32 bp | | |
| ACTB N-term | GCTATTCTCGCAGCTCACCAgttttagagctagaaatagcaagttaaaataaggctagtccgtt | 332 |
| PBS 13 RT 29 | atcaacttgaaaaagtggcaccgagtcggtgcGACGAGCGCGGCGATATCATCATCCA | |
| AttB 46 | TGGgggggtacaggtgccacattagttgtaccatttatTGAGCTGCGAGAA | |
| Staphylococcus_ | | |
| Iugdunensis | | |
| N920143_Att_rc | | |
| 36 bp | | |
| ACTB N-term | GCTATTCTCGCAGCTCACCAgttttagagctagaaatagcaagttaaaataaggctagtccgtt | 333 |
| PBS 13 RT 29 | atcaacttgaaaaagtggcaccgagtcggtgcGACGAGCGCGGCGATATCATCATCCA | |
| AttB 46 | TGGggtggtacaggtgccacattagttgtaccatttaTGAGCTGCGAGAA | |
| Staphylococcus_ | | |
| Iugdunensis | | |
| N920143_Att_rc | | |
| 34 bp | | |
| ACTB N-term | GCTATTCTCGCAGCTCACCAgttttagagctagaaatagcaagttaaaataaggctagtccgtt | 334 |
| PBS 13 RT 29 | atcaacttgaaaaagtggcaccgagtcggtgcGACGAGCGCGGCGATATCATCATCCA | |
| AttB 46 | TGGgtggtacaggtgccacattagttgtaccatttTGAGCTGCGAGAA | |
| Staphylococcus_ | | |
| Iugdunensis | | |
| N920143_Att_rc | | |
| 32 bp | | |
| ACTB N-term | GCTATTCTCGCAGCTCACCAgttttagagctagaaatagcaagttaaaataaggctagtccgtt | 335 |
| PBS 13 RT 29 | atcaacttgaaaaagtggcaccgagtcggtgcGACGAGCGCGGCGATATCATCATCCA | |
| AttB 46 Bacillus | TGGttatatttacaggaccaactggatctggaaaaacaaTGAGCTGCGAGAA | |
| _cytotoxicus | | |
| _NVH_ 391- | | |
| 98_Att 36 bp | | |
| ACTB N-term | GCTATTCTCGCAGCTCACCAgttttagagctagaaatagcaagttaaaataaggctagtccgtt | 336 |
| PBS 13 RT 29 | atcaacttgaaaaagtggcaccgagtcggtgcGACGAGCGCGGCGATATCATCATCCA | |
| AttB 46 | TGGtatatttacaggaccaactggatctggaaaaacaTGAGCTGCGAGAA | |
| Bacillus_ | | |
| cytotoxicus_ | | |
| NVH_391- | | |
| 98_Att 34 bp | | |
| ACTB N-term | GCTATTCTCGCAGCTCACCAgttttagagctagaaatagcaagttaaaataaggctagtccgtt | 337 |
| PBS 13 RT 29 | atcaacttgaaaaagtggcaccgagtcggtgcGACGAGCGCGGCGATATCATCATCCA | |
| AttB 46 Bacillus | TGGatatttacaggaccaactggatctggaaaaacTGAGCTGCGAGAA | |
| _cytotoxicus | | |
| _NVH_ 391- | | |
| 98_Att 32 bp | | |
| ACTB N-term | GCTATTCTCGCAGCTCACCAgttttagagctagaaatagcaagttaaaataaggctagtccgtt | 338 |
| PBS 13 RT 29 | atcaacttgaaaaagtggcaccgagtcggtgcGACGAGCGCGGCGATATCATCATCCA | |
| AttB 46 | TGGttgtttttccagatccagttggtcctgtaaatataaTGAGCTGCGAGAA | |
| Bacillus_ | | |
| cytotoxicus_ | | |
| NVH_ 391- | | |
| 98_Att_rc 36 bp | | |
| ACTB N-term | GCTATTCTCGCAGCTCACCAgttttagagctagaaatagcaagttaaaataaggctagtccgtt | 339 |
| PBS 13 RT 29 | atcaacttgaaaaagtggcaccgagtcggtgcGACGAGCGCGGCGATATCATCATCCA | |
| AttB 46 Bacillus | TGGtgtttttccagatccagttggtcctgtaaatataTGAGCTGCGAGAA | |
| _cytotoxicus | | |
| _NVH_391- | | |
| 98_Att_rc 34 bp | | |
| ACTB N-term | GCTATTCTCGCAGCTCACCAgttttagagctagaaatagcaagttaaaataaggctagtccgtt | 340 |
| PBS 13 RT 29 | atcaacttgaaaaagtggcaccgagtcggtgcGACGAGCGCGGCGATATCATCATCCA | |
| AttB 46 Bacillus | TGGgtttttccagatccagttggtcctgtaaatatTGAGCTGCGAGAA | |
| _cytotoxicus | | |
| _NVH_391- | | |
| 98_Att_rc 32 bp | | |
| Bacillus_cereus | GCTGTCTCCGCCGCCCGCCAgttttagagctagaaatagcaagttaaaataaggctagtccg | 341 |
| AH187_Att | ttatcaacttgaaaaagtggcaccgagtcggtgcAGTCGCCATGatatggggaagtgaatcagta | |
| _rc_36 LMNB1 | caaccgccacagtacCGGGCGGCG | |
| PBS 9 RT | | |
| 10 AttB 36 | | |
| atgRNA | | |
| Bacillus_cereus | GCGTATTGCCTGGAGGATGGgttttagagctagaaatagcaagttaaaataaggctagtccg | 342 |
| AH187_Att | ttatcaacttgaaaaagtggcaccgagtcggtgcGAACCACGCGGCGAATGCCGGCGT | |
| rc_36 NOLC1 | CCGCCatatggggaagtgaatcagtacaaccgccacagtacTCCTCCAGGCAATACG | |
| PBS 18 RT | CG | |
| 29 AttB 36 | | |
| atgRNA | | |
| Bacillus_cereus | GAGAAGCGGCGTCCGGGGCTAgttttagagctagaaatagcaagttaaaataaggctagtc | 343 |
| AH187_Att | cgttatcaacttgaaaaagtggcaccgagtcggtgcTCTTTGTCCAGAGTCACAGCCATA | |
| rc_36 SUPT 16H | atatggggaagtgaatcagtacaaccgccacagtacCCCCGGACGCCGC | |
| PBS 13 | | |
| RT 24 AttB 36 | | |
| atgRNA | | |
| Bacillus_cereus | GGGCACGGGGCCATGTACAAgttttagagctagaaatagcaagttaaaataaggctagtccg | 344 |
| AH187_Att | ttatcaacttgaaaaagtggcaccgagtcggtgcGGCGTCGGCAGCCCGATCCCGTTGa | |
| _rc_36 SRRM2 | tatggggaagtgaatcagtacaaccgccacagtacTACATGGCCCCGT | |
| PBS 13 RT | | |
| 24 AttB 36 | | |
| atgRNA | | |
| Bacillus_cereus_ | GTGTCAGGTGGGGCGGGGCTAgttttagagctagaaatagcaagttaaaataaggctagtc | 345 |
| AH187_Att | cgttatcaacttgaaaaagtggcaccgagtcggtgcGCTGGCTCCTCCCCTGGCACCATA | |
| _rc_36 DEPDC4 | atatggggaagtgaatcagtacaaccgccacagtacCCCCGCCCCACCTGACAC | |
| PBS 18 | | |
| RT 24 AttB 36 | | |
| atgRNA | | |
| Bacillus_cereus | GAGTGGGTCAGACGAGCAGGAgttttagagctagaaatagcaagttaaaataaggctagtc | 346 |
| AH187_Att | cgttatcaacttgaaaaagtggcaccgagtcggtgcCGACTCCTCCCCCATGCAGCCCTC | |
| _rc_36 NES PBS | CATCatatggggaagtgaatcagtacaaccgccacagtacTGCTCGTCTGACC | |
| 13 RT 28 | | |
| AttB 36 atgRNA | | |
| B. cereus | GCTGTCTCCGCCGCCCGCCAgttttagagctagaaatagcaagttaaaataaggctagtccg | 347 |
| LMNB1_PBS 9 | ttatcaacttgaaaaagtggcaccgagtcggtgcCGGGGGTCGCAGTCGCCATGatatgggg | |
| RT 20 AttB 36 | aagtgaatcagtacaaccgccacagtacCGGGCGGCG | |
| atgRNA | | |
| B. cereus | GGGCACGGGGCCATGTACAAgttttagagctagaaatagcaagttaaaataaggctagtccg | 348 |
| LMNB1_PBS 13 | ttatcaacttgaaaaagtggcaccgagtcggtgcCGGGGGTCGCAGTCGCCATGatatggg | |
| RT 20 AttB 36 | gaagtgaatcagtacaaccgccacagtacCGGGCGGCGGAGA | |
| atgRNA | | |
| B. cereus | GGGCACGGGGCCATGTACAAgttttagagctagaaatagcaagttaaaataaggctagtccg | 349 |
| LMNB1_PBS 13 | ttatcaacttgaaaaagtggcaccgagtcggtgcGCGGCGGCACGGGGGTCGCAGTCG | |
| RT 29 AttB 36 | CCATGatatggggaagtgaatcagtacaaccgccacagtacCGGGCGGCGGAGA | |
| atgRNA | | |
| B. cereus | GCGTATTGCCTGGAGGATGGgttttagagctagaaatagcaagttaaaataaggctagtccg | 350 |
| NOLC1_PBS 13 | ttatcaacttgaaaaagtggcaccgagtcggtgcGAACCACGCGGCGAATGCCGGCGTC | |
| RT 29 AttB 36 | CGCCatatggggaagtgaatcagtacaaccgccacagtacTCCTCCAGGCAAT | |
| atgRNA | | |
| B. cereus | GGGCACGGGGCCATGTACAAgttttagagctagaaatagcaagttaaaataaggctagtccg | 351 |
| NOLC1_PBS 13 | ttatcaacttgaaaaagtggcaccgagtcggtgcGGCGAATGCCGGCGTCCGCCatatgg | |
| RT 20 AttB 36 | ggaagtgaatcagtacaaccgccacagtacTCCTCCAGGCAAT | |
| atgRNA | | |
| B. cereus | GGGCACGGGGCCATGTACAAgttttagagctagaaatagcaagttaaaataaggctagtccg | 352 |
| NOLC1_PBS 18 | ttatcaacttgaaaaagtggcaccgagtcggtgcGGCGAATGCCGGCGTCCGCCatatgg | |
| RT 20 AttB 36 | ggaagtgaatcagtacaaccgccacagtacTCCTCCAGGCAATACGCG | |
| atgRNA | | |
| B. cereus | GGGCACGGGGCCATGTACAAgttttagagctagaaatagcaagttaaaataaggctagtccg | 353 |
| SRRM2_PBS 9 | ttatcaacttgaaaaagtggcaccgagtcggtgcGGCGTCGGCAGCCCGATCCCGTTGa | |
| RT 24 AttB 36 | tatggggaagtgaatcagtacaaccgccacagtacTACATGGCC | |
| atgRNA | | |
| B. cereus | GGGCACGGGGCCATGTACAAgttttagagctagaaatagcaagttaaaataaggctagtccg | 354 |
| SRRM2_PBS 9 | ttatcaacttgaaaaagtggcaccgagtcggtgcGATCCCGTTGatatggggaagtgaatca | |
| RT 10 AttB 36 | gtacaaccgccacagtacTACATGGCC | |
| atgRNA | | |
| B. cereus | GGGCACGGGGCCATGTACAAgttttagagctagaaatagcaagttaaaataaggctagtccg | 355 |
| SRRM2_PBS 13 | ttatcaacttgaaaaagtggcaccgagtcggtgcGATCCCGTTGatatggggaagtgaatca | |
| RT 10 AttB 36 | gtacaaccgccacagtacTACATGGCCCCGT | |
| atgRNA | | |
| Screen validation | GCTATTCTCGCAGCTCACCAgttttagagctagaaatagcaagttaaaataaggctagtccgtt | 356 |
| guides | atcaacttgaaaaagtggcaccgagtcggtgcgcgcggcgatatcatcatccatggatgatcc | |
| ACTB_1_11_24 | tgacgacggagaccgccgtcgtcgacaagcctgagctgcgag | |
| 38 | | |
| Screen validation | GCTATTCTCGCAGCTCACCAgttttagagctagaaatagcaagttaaaataaggctagtccgtt | 357 |
| guides | atcaacttgaaaaagtggcaccgagtcggtgccgatatcatcatccatggoggatgatcctgac | |
| ACTB_1_16_18 | gacggagaccgccgtcgtcgacaagccggctgagctgcgagaatag | |
| 43 | | |
| Screen validation | GCTGTCTCCGCCGCCCGCCAgttttagagctagaaatagcaagttaaaataaggctagtccg | 358 |
| guides | ttatcaacttgaaaaagtggcaccgagtcggtgcgcggcacgggggtcgcagtcgccatgat | |
| LMNB1_1_8_26 | gatcctgacgacggagaccgccgtcgtcgacaagcccgggcggc | |
| 38 | | |
| Screen validation | GCGTATTGCCTGGAGGATGGgttttagagctagaaatagcaagttaaaataaggctagtccg | 359 |
| guides | ttatcaacttgaaaaagtggcaccgagtcggtgcaatgccggcgtccgcccggatgatcctg | |
| NOLC1_1_15_16 | acgacggagaccgccgtcgtcgacaagccggctcctccaggcaatac | |
| 43 | | |
| Screen validation | GCGTATTGCCTGGAGGATGGgttttagagctagaaatagcaagttaaaataaggctagtccg | 360 |
| guides | ttatcaacttgaaaaagtggcaccgagtcggtgcggcgtccgccatgatcctgacgacggag | |
| NOLC1_1_14_10 | accgccgtcgtcgacaagcctcctccaggcaata | |
| _38 | | |
| Screen validation | GGGAAATGCATCTTGCACAAgttttagagctagaaatagcaagttaaaataaggctagtccgtt | 361 |
| guides | atcaacttgaaaaagtggcaccgagtcggtgcagcccctccatgctctctagctgttgccatt | |
| SERPIN_13_32 | gggcttgtcgacgacggcggtctccgtcgtcaggatcattgcaagatgcatt | |
| 38 | | |
| Screen validation | GTGTCAGGTGGGGCGGGGCTAgttttagagctagaaatagcaagttaaaataaggctagtc | 362 |
| guides | cgttatcaacttgaaaaagtggcaccgagtcggtgctggcaccataatgatcctgacgacg | |
| DEPDC4_8_10_3 | gagaccgccgtcgtcgacaagccccccgccc | |
| 8 | | |
| SERPIN Nicking | GTGGGGACAGCCCCGTCTCTgttttagagctagaaatagcaagttaaaataaggctagtccg | 363 |
| guide −107 guide | ttatcaacttgaaaaagtggcaccgagtcggtgc | |
| SERPIN Nicking | GCTCTTGGGAAAAAAACCCTAgttttagagctagaaatagcaagttaaaataaggctagtcc | 364 |
| guide −91 guide | gttatcaacttgaaaaagtggcaccgagtcggtgc | |
| SERPIN Nicking | GTCTTGGGAAAAAAACCCTAAgttttagagctagaaatagcaagttaaaataaggctagtccg | 365 |
| guide −90 guide | ttatcaacttgaaaaagtggcaccgagtcggtgc | |
| SERPIN Nicking | GAAAAAAACCCTAAGGGCTGgttttagagctagaaatagcaagttaaaataaggctagtccgt | 366 |
| guide −84 guide | tatcaacttgaaaaagtggcaccgagtcggtgc | |
| SERPIN Nicking | GCTGAGGATCCTTGTGAGTGTgttttagagctagaaatagcaagttaaaataaggctagtcc | 367 |
| guide −67 guide | gttatcaacttgaaaaagtggcaccgagtcggtgc | |
| SERPIN Nicking | GTGAGGATCCTTGTGAGTGTTgttttagagctagaaatagcaagttaaaataaggctagtcc | 368 |
| guide −66 guide | gttatcaacttgaaaaagtggcaccgagtcggtgc | |
| SERPIN Nicking | GGATCCTTGTGAGTGTTGGGgttttagagctagaaatagcaagttaaaataaggctagtccgt | 369 |
| guide −63 guide | tatcaacttgaaaaagtggcaccgagtcggtgc | |
| SERPIN Nicking | GATCCTTGTGAGTGTTGGGTgttttagagctagaaatagcaagttaaaataaggctagtccgtt | 370 |
| guide −62 guide | atcaacttgaaaaagtggcaccgagtcggtgc | |
| SERPIN Nicking | GTTGGGTGGGAACAGCTCCCgttttagagctagaaatagcaagttaaaataaggctagtccg | 371 |
| guide −49 guide | ttatcaacttgaaaaagtggcaccgagtcggtgc | |
| SERPIN Nicking | GGGTGGGAACAGCTCCCAGGgttttagagctagaaatagcaagttaaaataaggctagtcc | 372 |
| guide −46 guide | gttatcaacttgaaaaagtggcaccgagtcggtgc | |
| SERPIN Nicking | GCTTCTGTGCAGCAGTTTCCCgttttagagctagaaatagcaagttaaaataaggctagtcc | 373 |
| guide +34 guide | gttatcaacttgaaaaagtggcaccgagtcggtgc | |
| SERPIN Nicking | GTTTCCCTGGCCACTAAATAGgttttagagctagaaatagcaagttaaaataaggctagtccg | 374 |
| guide +48 guide | ttatcaacttgaaaaagtggcaccgagtcggtgc | |
| SERPIN Nicking | GTTCCCTGGCCACTAAATAGTgttttagagctagaaatagcaagttaaaataaggctagtccg | 375 |
| guide +49 guide | ttatcaacttgaaaaagtggcaccgagtcggtgc | |
| SERPIN Nicking | GATTAGATAGAAGCCCTCCAgttttagagctagaaatagcaagttaaaataaggctagtccgtt | 376 |
| guide +71 guide | atcaacttgaaaaagtggcaccgagtcggtgc | |
| SERPIN Nickingv | GATTAGATAGAAGCCCTCCAAgttttagagctagaaatagcaagttaaaataaggctagtcc | 377 |
| guide +72 guide | gttatcaacttgaaaaagtggcaccgagtcggtgc | |

Additional attachment sites suitable for use with the Serine integrases described in International Patent Publication No. WO 2023/070031A2, such as SEQ ID NOs: 1-16 and 163-1162 and 3166-3175 of WO2023/070031, are also disclosed in WO2023/070031 (which PCT publication is hereby incorporated by reference in its entirety).

Further suitable attachment sites for integrase (such as the eLSR described herein) suitable for use with the invention described herein are described in WO2023/177424 (which PCT publication is hereby incorporated by reference in its entirety).

### 6.8. Integrases/Recombinases and Integration/Recombination Sites

In typical embodiments, the co-delivery system described herein contains an integrase (such as the eLSR described herein) and/or a recombinase. In some embodiments, the co-delivery system includes an integrase (such as the eLSR described herein) and/or a recombinase packaged in a LNP. In one embodiment, the co-delivery system includes a polynucleotide encoding an integrase (such as the eLSR described herein) and/or a recombinase. In some embodiments, the co-delivery system includes an integrase (such as the eLSR described herein) or a recombinase packaged in a vector (e.g., a viral vector). In some embodiments, the co-delivery system includes at least a first integrase (e.g., a first integrase and a second integrase) and/or at least a first recombinase (e.g., a first recombinase and a second recombinase).

In some embodiments, the integration enzyme (e.g., the integrase or recombinase) is selected from the group consisting of Dre, Vika, Bxb1, φpC31, RDF, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, cpRV, retrotransposases encoded by a Tcl/mariner family member including but not limited to retrotransposases encoded by LI, Tol2, Tel, Tc3, Himar 1 (isolated from the hom fly, Haematobia irritans), Mos1 (Mosaic element of Drosophila mauritiana), and Minos, and any mutants thereof. As can be used herein, Xu et al describes methods for evaluating integrase activity in E. coli and mammalian cells and confirmed at least R4, φC31, φBT1, Bxb1, SPBc, TP901-1 and WP integrases to be active on substrates integrated into the genome of HT1080 cells (Xu et al., 2013, Accuracy and efficiency define Bxb1 integrase as the best of fifteen candidate serine recombinases for the integration of DNA into the human genome. BMC Biotechnol. 2013 Oct. 20; 13:87. doi: 10.1186/1472-6750-13-87). Durrant describes new large serine recombinases (LSRs) divided into three classes distinguished from one another by efficiency and specificity, including landing pad LSRs which outperform wild-type Bxb1 in episomal and chromosomal integration efficiency, LSRs that achieve both efficient and site-specific integration without a landing pad, and multi-targeting LSRs with minimal site-specificity. Additionally, embodiments can include any serine recombinase such as BceINT, SSCINT, SACINT, and INT10 (see Ionnidi et al., 2021; Drag-and-drop genome insertion without DNA cleavage with CRISPR directed integrases. bioRxiv 2021.11.01.466786, doi.org/10.1101/2021.11.01.466786).

In some embodiments, the integration site can be selected from an attB site, an attP site, an attL site, an attR site, a lox71 site a Vox site, or a FRT site. In instances in this disclosure that refer to a Cre-lox system, the Cre-lox system is referred to either as a control for programmable gene insertion or as a tool for a recombinase-mediated event separate and distinct from insertion of the donor polynucleotide template (or exogenous nucleic acid) into the integrated recognition site.

It will be appreciated that desired activity of integrases, transposases and the like can depend on nuclear localization. In certain embodiments, prokaryotic enzymes are adapted to modulate nuclear localization. In certain embodiments, eukaryotic or vertebrate enzymes are adapted to modulate nuclear localization. In certain embodiments, the invention provides fusion or hybrid proteins. Such modulation can comprise addition or removal of one or more nuclear localization signal (NLS) and/or addition or removal of one or more nuclear export signal (NES). Xu et al compared derivatives of fourteen serine integrases that either possess or lack a nuclear localization signal (NLS) to conclude that certain integrases benefit from addition of an NLS whereas others are transported efficiently without addition, and a major determinant of activity in yeast and vertebrate cells is avoidance of toxicity. (Xu et al., 2016, Comparison and optimization of ten phage encoded serine integrases for genome engineering in Saccharomyces cerevisiae. BMC Biotechnol. 2016 Feb. 9; 16:13. doi: 10.1186/s12896-016-0241-5). Ramakrishnan et al. systematically studied the effect of different NES mutants developed from mariner-like elements (MLEs) on transposase localization and activity and concluded that nuclear export provides a means of controlling transposition activity and maintaining genome integrity. (Ramakrishnan et al. Nuclear export signal (NES) of transposases affects the transposition activity of mariner-like elements Ppmar1 and Ppmar2 of moso bamboo. Mob DNA. 2019 Aug. 19; 10:35. doi:10.1186/s13100-019-0179-y). The methods and constructs are used to modulate nuclear localization of system components of the invention.

In typical embodiments, the integrase used herein is selected from below (Table 10).

TABLE 10
Integrases

| | | | | protein | | | | | | | | | |
|---|---|---|---|---|---|---|---|---|---|---|---|---|---|
| | | | nucleo- | acc- | inter- | | | | | | | | |
| | SRA | bio | tide | ession | nal | | Alt- | | | | SEQ | | |
| Data- | acc- | project_ | acc- | or | protein | Proposed | ernative | organism/ | des- | | ID | | |
| base | ession | acc | ession | ORF ID | ID | names | names | source | cription | Sequence | NO: | Length | Group |
| ENA | SR | PRJ | NA | NA | N189929_ | SsuI | NA | human | stool | MEKNRAVLYLRLSKEDVD | 378 | 527 | INTc |
| | S12 | EB2 | | | 49_54 | NT | | gutmeta | sample | KVNKGDDSSSIKSQRLLLT | | | |
| | 052 | 627 | | | | | | genome | from | DFALERGFKIVGVYSDDDE | | | |
| | 98 | 7 | | | | | | | male in | SGLYDDRPDFERMMTDAK | | | |
| | | | | | | | | | USA | LDEFDIIIAKTQSRFSRNME | | | |
| | | | | | | | | | | HIEKYLHHDLPNLGIRFIGA | | | |
| | | | | | | | | | | VDGVDTESDENKKSRQIN | | | |
| | | | | | | | | | | GLVNEWYCEDLSKNIRSA | | | |
| | | | | | | | | | | FKAKMKDGQFLGSSCPYG | | | |
| | | | | | | | | | | YKKDPQNHNHLVVDDYAA | | | |
| | | | | | | | | | | KVVQKIFNLYLEGYGKAKI | | | |
| | | | | | | | | | | GSILSSEGILIPTLYKKDILK | | | |
| | | | | | | | | | | QNYHNSKALDTTQNWSY | | | |
| | | | | | | | | | | QTIHTILNNEVYLGHLIQNK | | | |
| | | | | | | | | | | VNTMSYKDKNKRILPKEK | | | |
| | | | | | | | | | | WIIVRNTHEPIITEEMFQDV | | | |
| | | | | | | | | | | QKLQKNRTRSVENIEPNGL | | | |
| | | | | | | | | | | FSGLIFCADCKHAMSRKYA | | | |
| | | | | | | | | | | RRGEKGFVGYVCKTYKTQ | | | |
| | | | | | | | | | | GKNFCESHSIDYDELEEAV | | | |
| | | | | | | | | | | LFSIKNEARSILQQEEIDEL | | | |
| | | | | | | | | | | RKVQAYDETKSYYEMQLE | | | |
| | | | | | | | | | | NIKSRMEKIEKYKKKTYDN | | | |
| | | | | | | | | | | YMDDLISRDDYKKYVTEYD | | | |
| | | | | | | | | | | KEIGGLKQQQELINSKTDL | | | |
| | | | | | | | | | | EKEISTQYDEWVEAFINYV | | | |
| | | | | | | | | | | DIDKLTREIVIELIEKIEVNK | | | |
| | | | | | | | | | | DGSINIYYKFKNPYIS | | | |
| ENA | ERS | PRJ | NA | NA | N190156_ | SssI | NA | human | stool | MNTVIYARYSAGPRQTDQ | 379 | 510 | INTd |
| | 396 | EB2 | | | 234_12 | NT | | gut | sample | SIDGQLRVCTEFCKQRGLT | | | |
| | 461 | 628 | | | | | | metage | from | VVDTYCDRHISGRTDERP | | | |
| | | 0 | | | | | | nome | Spain | EFQRLIADAKAHKFEAVVV | | | |
| | | | | | | | | | | YKTDRFARNKYDSAIYKRE | | | |
| | | | | | | | | | | LRRNGIQIFYAAEAIPEGPE | | | |
| | | | | | | | | | | GIILESLMEGLAEYYSAELA | | | |
| | | | | | | | | | | QKIKRGLNESALKCQSLGS | | | |
| | | | | | | | | | | GRPLGYTVDEQKHFQIDP | | | |
| | | | | | | | | | | ESSQAVKTIFEMYIKGESN | | | |
| | | | | | | | | | | AAICDYLNARGLRTSQGNL | | | |
| | | | | | | | | | | FNKNSINRIIKNRKYIGEYR | | | |
| | | | | | | | | | | YNDIVVEGGMPAIISKETFC | | | |
| | | | | | | | | | | MAQAEMERRRTHRAPVS | | | |
| | | | | | | | | | | PKAEYLLAGKLFCGHCKG | | | |
| | | | | | | | | | | PMQGVSGTGKSGNKWYY | | | |
| | | | | | | | | | | YYCANTRGKERTCDKKQV | | | |
| | | | | | | | | | | SRDRLEKAVVDFTVRYILQ | | | |
| | | | | | | | | | | ENVLEELSKKVYAAQERQ | | | |
| | | | | | | | | | | NNTASEIAFYEKKLAENKK | | | |
| | | | | | | | | | | AIANILRAIESGAMTQALPA | | | |
| | | | | | | | | | | RLQELENEQTVIQGELSYL | | | |
| | | | | | | | | | | KGARLAFTEDQILFALLQH | | | |
| | | | | | | | | | | LDPRPGESERDYHRRIITD | | | |
| | | | | | | | | | | FVSEVYLYDDRMLIYFNISS | | | |
| | | | | | | | | | | ADGKLKHADLSAIESGVFD | | | |
| | | | | | | | | | | AGLISSSSRASSFSTRCALI | | | |
| ENA | ER | PRJ | NA | NA | N191352_ | SscI | NA | human | stool | MNEKNLEIGAAYIRVSTDD | 380 | 482 | INTd |
| | S10 | EB2 | | | 143_72 | NT | | gutmeta | sample | QTELSPDAQLRVILEAAKK | | | |
| | 158 | 683 | | | | | | genome | from | DGIIIPQEFVFMEDRGRSG | | | |
| | 37 | 2 | | | | | | | China | RRADNRPEFQRMISTARQ | | | |
| | | | | | | | | | | NPSPFRYLYLWKFSRFAR | | | |
| | | | | | | | | | | NQEESAFYKGILRKKCGVT | | | |
| | | | | | | | | | | IKSVSEPIMEGMFGRLVEM | | | |
| | | | | | | | | | | IIEWSDEFYSVNLSGEVLR | | | |
| | | | | | | | | | | GMTQKALEHGYQLTPCLG | | | |
| | | | | | | | | | | YDAVGHGRPYVINEEQYQI | | | |
| | | | | | | | | | | VEFIHRSFFDGKDMTWIAR | | | |
| | | | | | | | | | | EANRRGYHTRRGNPFDTR | | | |
| | | | | | | | | | | AVRIILTNSFYVGLVKWND | | | |
| | | | | | | | | | | VTFQGTHECRESVTSVFS | | | |
| | | | | | | | | | | ANQERLNRIHRPRGRRQA | | | |
| | | | | | | | | | | SSCKHWLSGLLKCSICGA | | | |
| | | | | | | | | | | SLGYNQTKDLTKRGHAFQ | | | |
| | | | | | | | | | | CWKYTKGIHPGSCSVSSL | | | |
| | | | | | | | | | | KAEAAVLESLQMILETGEV | | | |
| | | | | | | | | | | EYTYEQREKHLDDNKLTLI | | | |
| | | | | | | | | | | QKSLERLDTKELRIREAYE | | | |
| | | | | | | | | | | SGIDTLDEFKTNKARLQRE | | | |
| | | | | | | | | | | RDQLMEELEELHSQEEPE | | | |
| | | | | | | | | | | DVPGKEILIERIQNVYDLLQ | | | |
| | | | | | | | | | | SPDVDNDDKGNAVRSIIKK | | | |
| | | | | | | | | | | IVYIKESKTFCFYYYV | | | |
| ENA | ER | PRJ | NA | NA | N191533_ | Ssc2 | NA | human | stool | MERTIKVIQPGTVKIPTKKR | 381 | 406 | INTc |
| | S12 | EB2 | | | 224_76 | INT | | gutmeta | sample | VAAYARVSSGKDAMLHSL | | | |
| | 896 | 692 | | | | | | genome | from | SAQVSYYSNMIQQKNEWS | | | |
| | 77 | 4 | | | | | | | China | YVGIYADEAITGTKDRRVE | | | |
| | | | | | | | | | | FNRLIQDCTDGKIDMIITKSI | | | |
| | | | | | | | | | | SRFARNTLTMLEVVRKLKN | | | |
| | | | | | | | | | | INVDVYFEKENIHSISGDGE | | | |
| | | | | | | | | | | LMLTILASFAQEESRSVSE | | | |
| | | | | | | | | | | NCKWRIRKGFEQGELINLR | | | |
| | | | | | | | | | | FLYGYRINKGKIEIYEKEAEI | | | |
| | | | | | | | | | | VRMIFDDYLNGEGCTRIGN | | | |
| | | | | | | | | | | KLRKMKVNKLRGGMWNS | | | |
| | | | | | | | | | | ERVVDIIKNEKYTGNALLQ | | | |
| | | | | | | | | | | KKYVKDHLSKKLVRNKGIL | | | |
| | | | | | | | | | | TQYYAEGTHPAIIDIKTFEIA | | | |
| | | | | | | | | | | QKIMEANRTKFQGKCGSN | | | |
| | | | | | | | | | | RYLFTSKIECGICGKNYRH | | | |
| | | | | | | | | | | KDREGKSTWVCANHLKYG | | | |
| | | | | | | | | | | NSRCIAKPLNEEKLKKLINE | | | |
| | | | | | | | | | | ALELKYFDEEIFIRNIKRIKV | | | |
| | | | | | | | | | | TGNQTIEFILKDGKVIEEGM | | | |
| | | | | | | | | | | I | | | |
| ENA | ERS | PRJ | NA | NA | N203911_ | SsdI | NA | human | stool | MKKIKIDRAIQERPATRKQ | 382 | 401 | INTc |
| | 265 | EB2 | | | 45186_6 | NT | | gut | sample | TRNEKIRQSLTEHVDVQVI | | | |
| | 582 | 824 | | | | | | metage | from | PAITDREGYEKPKLRVCAY | | | |
| | 7 | 5 | | | | | | nome | Denmark | CRVSTDMDTQALSYELQV | | | |
| | | | | | | | | | | QNYTDYIRGNDEWRFAGI | | | |
| | | | | | | | | | | YADRGISGTSLKHRDEFN | | | |
| | | | | | | | | | | RMIEDCKAGKIDLIITKAVT | | | |
| | | | | | | | | | | RFARNVLDCISTIRMLKQL | | | |
| | | | | | | | | | | EHPVAVYFETERINTLDTT | | | |
| | | | | | | | | | | SETYLGLISLFAQGESESK | | | |
| | | | | | | | | | | SESLKWSYIRRWKRGTGI | | | |
| | | | | | | | | | | YPAWSLLGYEMGEDGKW | | | |
| | | | | | | | | | | QIVEAEAELVRIIYDMYLNG | | | |
| | | | | | | | | | | YSSPQIAEILTRSGVPTATN | | | |
| | | | | | | | | | | QTVWSSGGVLGILRNEKY | | | |
| | | | | | | | | | | CGNVLCQKTMTVDVFSHK | | | |
| | | | | | | | | | | AIKNTGQKTQYFIEGHHDP | | | |
| | | | | | | | | | | IILRSDWDRVQQMIDEKYY | | | |
| | | | | | | | | | | RKRRGRRTKPRIVLKGCLA | | | |
| | | | | | | | | | | GFTQIDLDWDEDDIARIFY | | | |
| | | | | | | | | | | STTPAAEVATPAMADHIEII | | | |
| | | | | | | | | | | KVKGEN | | | |
| ENA | SRS | PRJ | NA | NA | N208621_ | SmcI | NA | human | sample | MKTAAAYIRVSTDDQVEYS | 383 | 476 | INTd |
| | 294 | EB3 | | | 9_15 | NT | | gut | from | PDSQIKLIRDYAKRNDYILP | | | |
| | 942 | 004 | | | | | | metage | 72- | DEFIFRDDGISGKSAKHRP | | | |
| | | | | | | | | nome | year-old | EFTKMIALAKSPEHPFDAIL | | | |
| | | 16 | | | | | | | male | VWKFSRFARNQEESIVFK | | | |
| | | | | | | | | | from | NILRKIGVEVRSVSEPISED | | | |
| | | | | | | | | | China | PFGSLVERIIEWTDEYYIIN | | | |
| | | | | | | | | | | LSGEVKRGMLEKISRGQP | | | |
| | | | | | | | | | | WPPPVGYKMENGQYIPD | | | |
| | | | | | | | | | | ENAHFIKEIFEAYAAGEGA | | | |
| | | | | | | | | | | RHIAQRLAAQGCLTKRGN | | | |
| | | | | | | | | | | PIDNRFVDYVLHNPVYIGK | | | |
| | | | | | | | | | | LRWSVNSHAASSRHYDSA | | | |
| | | | | | | | | | | DIIVFDGTHEPLISSELWES | | | |
| | | | | | | | | | | VQKRLHEVKTLYPKYQRR | | | |
| | | | | | | | | | | EQPVSFMLKGLVRCSSCG | | | |
| | | | | | | | | | | STLCYCRTSEPSLQCHSY | | | |
| | | | | | | | | | | ARGSCRQSHSINIATANEA | | | |
| | | | | | | | | | | VIKGLQLAVDKLDFAIAPAK | | | |
| | | | | | | | | | | PHYSADAPGTNKLLAAEY | | | |
| | | | | | | | | | | KKMERIKAAYANGTDTLEE | | | |
| | | | | | | | | | | YAANKKKISAEIARLEAELQ | | | |
| | | | | | | | | | | QESNVKPINKKAFAKRVSE | | | |
| | | | | | | | | | | IIKYISDPHNSEAAKNQALR | | | |
| | | | | | | | | | | TVISYIIFDRAATTFNIIFHF | | | |
| Meta | NA | NA | NA | NA | N675015_ | UhmI | NA | urban | NA | MKIAIYARKSKYSPTGESV | 384 | 550 | INTd |
| SUB | | | | | 95_5 | NT | | human | | ENQIQLCKEYLQAKYKSET | | | |
| | | | | | | | | micro- | | LEIDEYKDEGYSGGNTNR | | | |
| | | | | | | | | biome | | PDFKKLIAQIEDYDMLICYR | | | |
| | | | | | | | | | | LDRISRNVADFSSTLTLLQ | | | |
| | | | | | | | | | | NNKCDFVSIKEQFDTTSPM | | | |
| | | | | | | | | | | GRAMIYISSVFAQLERETIA | | | |
| | | | | | | | | | | ERIRDNMMELAKMGRWL | | | |
| | | | | | | | | | | GGTIPMGFDSEPITFIDEN | | | |
| | | | | | | | | | | MKERSMTKLIPNVEELKVI | | | |
| | | | | | | | | | | ELIYEKYLQLGSMGKVVTY | | | |
| | | | | | | | | | | LLQNNIKTKKGKDFTLGSIK | | | |
| | | | | | | | | | | VILTNPIYVKANQEVVNHLK | | | |
| | | | | | | | | | | TQGITICGDVDGKKALLTY | | | |
| | | | | | | | | | | NKTTGISNDVGTKTIVKDK | | | |
| | | | | | | | | | | SEWIAAVANHKGIIPADKW | | | |
| | | | | | | | | | | LQAQNIKDKNKDSFPALGR | | | |
| | | | | | | | | | | SNTTIASRVLRCDKCESTM | | | |
| | | | | | | | | | | GVTHGHINPVTGKKHYYY | | | |
| | | | | | | | | | | NCTLKKRSKGVRCDNKPA | | | |
| | | | | | | | | | | KAAEVDEAILITLENMFKAK | | | |
| | | | | | | | | | | SSIIDNLKAKNKARRIEMIS | | | |
| | | | | | | | | | | SNRVDVINKIIEDKTKQIDN | | | |
| | | | | | | | | | | LVNKLSLDDDLTDILFKKIK | | | |
| | | | | | | | | | | GLKAEIKELEDELLTLTSDN | | | |
| | | | | | | | | | | IKLNEDEVVLDFTEKLLEKC | | | |
| | | | | | | | | | | SIIRTLDILEQQQIVDALIPL | | | |
| | | | | | | | | | | VTWNGDTEVLNIYPLGSPE | | | |
| | | | | | | | | | | LELKEAESKKK | | | |
| Sega | NA | PRJ | NA | NA | N684346_ | SacI | NA | human | stool | MKEKVSERKTGAIYIRVST | 385 | 493 | INTd |
| ta- | | NA4 | | | 90_69 | NT | | gut | sample | DKQEELSPDAQLRLLLDYA | | | |
| Pas | | 224 | | | | | | metage | from | KKDSIDVPKEYIFQDNGIS | | | |
| olli | | 34 | | | | | | nome | adult in | GRKANKRPAFQNMIALAK | | | |
| | | | | | | | | | China | SKEHPIDTIIVWKFSRFARN | | | |
| | | | | | | | | | | QEESIVYKSLLKKNNVDVV | | | |
| | | | | | | | | | | SVSEPLIDGPFGSLIERIIE | | | |
| | | | | | | | | | | WMDEYYSIRLSGEVMRG | | | |
| | | | | | | | | | | MTQNAMRGHYQSDAPIGY | | | |
| | | | | | | | | | | TSPGDKKPPVINPDTVQIP | | | |
| | | | | | | | | | | LMIKDMFLSGSTQLQIARK | | | |
| | | | | | | | | | | LNDSGYRTKRGNLWDAR | | | |
| | | | | | | | | | | GVRYVLENPFYIGKSRWN | | | |
| | | | | | | | | | | YTERGRRLKPADEVIYADG | | | |
| | | | | | | | | | | NWEALWDEDTFKEIQKRL | | | |
| | | | | | | | | | | ALNMRKSKSRDISAAKHW | | | |
| | | | | | | | | | | LSGLLICSSCGGTLAFGGA | | | |
| | | | | | | | | | | HNMRGFQCWKYSKGFCS | | | |
| | | | | | | | | | | ESHYISTGPIEKMVLEYLEA | | | |
| | | | | | | | | | | VMHSPALSYTVISSSSVDA | | | |
| | | | | | | | | | | SSKLSDLERQLQKIDAKEK | | | |
| | | | | | | | | | | RIKAAYLNEIDTLEEYKANK | | | |
| | | | | | | | | | | TALEEERRTVEKEIEELTLS | | | |
| | | | | | | | | | | DVKYSKEDLDKKMKQNIS | | | |
| | | | | | | | | | | DLLRVLRDESADYIQKGN | | | |
| | | | | | | | | | | MMRNVVDHIVFNRKNTSL | | | |
| | | | | | | | | | | DVFLKLVV | | | |
| Sega | ER | PRJ | NA | NA | N687611_ | RsaI | NA | human | rectal | MKITKKQPLRPRGRSEDK | 386 | 404 | INTc |
| ta- | R | EB1 | | | 90_68 | NT | | gut | swab | RQSTKNVIRDAYINGPQKE | | | |
| Pas | 113 | 153 | | | | | | metage | from | VQIIPAKRDMEAETEKKKL | | | |
| oLi | 686 | 2 | | | | | | nome | adult in | RVCAYCRVSTDEDTQASS | | | |
| | 4 | | | | | | | | Isreal | YELQVQNYTRMIRENPEW | | | |
| | | | | | | | | | | EFAGIFADEGISGTSVLHR | | | |
| | | | | | | | | | | EHFLEMIEKCKAGEIDLIITK | | | |
| | | | | | | | | | | QVSRFARNVLDSLNYIFML | | | |
| | | | | | | | | | | RKLDPPVGVYFETEKLNTL | | | |
| | | | | | | | | | | DKSSDMVITVLSLVAQSES | | | |
| | | | | | | | | | | EQKSNSLKWSFKRRRAQ | | | |
| | | | | | | | | | | GLGIYPSWALLGYRLDDEK | | | |
| | | | | | | | | | | NWEIVEDEADIVRTIYSLYL | | | |
| | | | | | | | | | | DGYSSTQIAELLTKSGIPTV | | | |
| | | | | | | | | | | KGLSVWSSGSVLGILKNEK | | | |
| | | | | | | | | | | FCGDALCQKTVTIDFFTHK | | | |
| | | | | | | | | | | SVKNNGIEPQYFVEGHHIP | | | |
| | | | | | | | | | | IIEKNDWLLAQQIRKERRY | | | |
| | | | | | | | | | | RKRRSTHRKPRIVVKGALS | | | |
| | | | | | | | | | | GFMIVDTSWDEEYVDSLLI | | | |
| | | | | | | | | | | SATQKPEPAPVIAEEDENF | | | |
| | | | | | | | | | | IVIEKE | | | |
| Seg | ER | PRJ | NA | NA | N687663_ | Rsa2 | NA | human | rectal | MADIQPVKNGALYIRVSTH | 387 | 498 | INTd |
| ata- | R11 | EB1 | | | 53_29 | INT | | gut | swab | LQEELSPDAQKRLLMEYA | | | |
| Pas | 367 | 153 | | | | | | metage | from | EAHNIIVLKEHIYIDSGISGR | | | |
| o lli | 37 | 2 | | | | | | nome | adult in | SARQRPQFNNMIAEAKSK | | | |
| | | | | | | | | | Israel | EHPFDVILVWKYSRFARN | | | |
| | | | | | | | | | | QEESIVYKSMLKRENVDVI | | | |
| | | | | | | | | | | SVSEPISDDPFGSLIERIIE | | | |
| | | | | | | | | | | WMDEYYSIRLSGEVSRGM | | | |
| | | | | | | | | | | AENAMRGNYQARPPLGY | | | |
| | | | | | | | | | | RIPGYRQTPVIVPEEAELIQ | | | |
| | | | | | | | | | | LIFDLYTEKKMGIFEIVRYL | | | |
| | | | | | | | | | | NEHGYQTGHKKPFQRRSV | | | |
| | | | | | | | | | | TYILKNPTYIGKTIWNQHD | | | |
| | | | | | | | | | | QDHKLRDKSEWIIADGKHE | | | |
| | | | | | | | | | | PIISKEQFDKAQKRIESTYK | | | |
| | | | | | | | | | | PAYRKPTSVCHHWLSSLL | | | |
| | | | | | | | | | | KCSSCGRTLVVKRTASKK | | | |
| | | | | | | | | | | KDRMYVNFQCYGYQKGIC | | | |
| | | | | | | | | | | NTNQSISAIKLEPVIMHALE | | | |
| | | | | | | | | | | DAMTSGKIHFDVLNPTTLD | | | |
| | | | | | | | | | | SSQKQQFLTRLNEIEKKEE | | | |
| | | | | | | | | | | RIKRAYRDGIDTLEEYKEN | | | |
| | | | | | | | | | | KSIIQTEKEMLLKKIEHIEEP | | | |
| | | | | | | | | | | ALSPEEAKPIMMDRIKNVY | | | |
| | | | | | | | | | | EIITNPDIGMEEKNKAARSII | | | |
| | | | | | | | | | | EKIVFDRATGSVNIFFYLAH | | | |
| | | | | | | | | | | CP | | | |
| NCBI | NA | NA | NC_ | NP_ | NA | Bxb | Bxb1 | Myco- | NA | MRALVVIRLSRVTDATTSP | 388 | 501 | INTa |
| | | | 0026 | 07530 | | NT | inte- | bacterium | | ERQLESCQQLCAQRGWD | | | |
| | | | 56.1 | 2.1 | | | grase | phage | | WGVAEDLDVSGAVDPFD | | | |
| | | | | | | | | Bxb1 | | RKRRPNLARWLAFEEQPF | | | |
| | | | | | | | | | | DVIVAYRVDRLTRSIRHLQ | | | |
| | | | | | | | | | | QLVHWAEDHKKLVVSATE | | | |
| | | | | | | | | | | AHFDTTTPFAAVVIALMGT | | | |
| | | | | | | | | | | VAQMELEAIKERNRSAAHF | | | |
| | | | | | | | | | | NIRAGKYRGSLPPWGYLP | | | |
| | | | | | | | | | | TRVDGEWRLVPDPVQRE | | | |
| | | | | | | | | | | RILEVYHRVVDNHEPLHLV | | | |
| | | | | | | | | | | AHDLNRRGVLSPKDYFAQ | | | |
| | | | | | | | | | | LQGREPQGREWSATALKR | | | |
| | | | | | | | | | | SMISEAMLGYATLNGKTVR | | | |
| | | | | | | | | | | DDDGAPLVRAEPILTREQL | | | |
| | | | | | | | | | | EALRAELVKTSRAKPAVST | | | |
| | | | | | | | | | | PSLLLRVLFCAVCGEPAYK | | | |
| | | | | | | | | | | FAGGGRKHPRYRCRSMG | | | |
| | | | | | | | | | | FPKHCGNGTVAMAEWDA | | | |
| | | | | | | | | | | FCEEQVLDLLGDAERLEKV | | | |
| | | | | | | | | | | WVAGSDSAVELAEVNAEL | | | |
| | | | | | | | | | | VDLTSLIGSPAYRAGSPQR | | | |
| | | | | | | | | | | EALDARIAALAARQEELEG | | | |
| | | | | | | | | | | LEARPSGWEWRETGQRF | | | |
| | | | | | | | | | | GDWWREQDTAAKNTWLR | | | |
| | | | | | | | | | | SMNVRLTFDVRGGLTRTID | | | |
| | | | | | | | | | | FGDLQEYEQHLRLGSVVE | | | |
| | | | | | | | | | | RL HTGMS* | | | |
| NCBI | NA | NA | NC_ | NP_ | NA | Tp9I | TP901- | Lacto- | NA | MTKKVAIYTRVSTTNQAEE | 389 | 486 | INTd |
| | | | 0027 | 11266 | | NT | 1inte- | coccus | | GFSIDEQIDRLTKYAEAMG | | | |
| | | | | | | | grase | phage | | WQVSDTYTDAGFSGAKLE | | | |
| | | | 47.1 | 4.1 | | | | TP901- | | RPAMQRLINDIENKAFDTV | | | |
| | | | | | | | | 1 | | LVYKLDRLSRSVRDTLYLV | | | |
| | | | | | | | | | | KDVFTKNKIDFISLNESIDT | | | |
| | | | | | | | | | | SSAMGSLFLTILSAINEFER | | | |
| | | | | | | | | | | ENIKERMTMGKLGRAKSG | | | |
| | | | | | | | | | | KSMMWTKTAFGYYHNRK | | | |
| | | | | | | | | | | TGILEIVPLQATIVEQIFTDY | | | |
| | | | | | | | | | | LSGISLTKLRDKLNESGHIG | | | |
| | | | | | | | | | | KDIPWSYRTLRQTLDNPVY | | | |
| | | | | | | | | | | CGYIKFKDSLFEGMHKPIIP | | | |
| | | | | | | | | | | YETYLKVQKELEERQQQT | | | |
| | | | | | | | | | | YERNNNPRPFQAKYMLSG | | | |
| | | | | | | | | | | MARCGYCGAPLKIVLGHK | | | |
| | | | | | | | | | | RKDGSRTMKYHCANRFPR | | | |
| | | | | | | | | | | KTKGITVYNDNKKCDSGTY | | | |
| | | | | | | | | | | DLSNLENTVIDNLIGFQEN | | | |
| | | | | | | | | | | NDSLLKIINGNNQPILDTSS | | | |
| | | | | | | | | | | FKKQISQIDKKIQKNSDLYL | | | |
| | | | | | | | | | | NDFITMDELKDRTDSLQAE | | | |
| | | | | | | | | | | KKLLKAKISENKENDSTDV | | | |
| | | | | | | | | | | FELVKTQLGSIPINELSYDN | | | |
| | | | | | | | | | | KKKIVNNLVSKVDVTADNV | | | |
| | | | | | | | | | | DIIFKFQLA* | | | |
| NCBI | NA | NA | NC_ | NP- | NA | Bt1I | PhiBT | Strepto- | NA | MSPFIAPDVPEHLLDTVRV | 390 | 595 | INTa |
| | | | 004664. | 81374 | | NT | inte- | myces | | FLYARQSKGRSDGSDVST | | | |
| | | | 2 | 4.2 | | | grase | virus | | EAQLAAGRALVASRNAQG | | | |
| | | | | | | | | phiBT1 | | GARWVVAGEFVDVGRSG | | | |
| | | | | | | | | | | WDPNVTRADFERMMGEV | | | |
| | | | | | | | | | | RAGEGDVVVVNELSRLTR | | | |
| | | | | | | | | | | KGAHDALEIDNELKKHGVR | | | |
| | | | | | | | | | | FMSVLEPFLDTSTPIGVAIF | | | |
| | | | | | | | | | | ALIAALAKQDSDLKAERLK | | | |
| | | | | | | | | | | GAKDEIAALGGVHSSSAPF | | | |
| | | | | | | | | | | GMRAVRKKVDNLVISVLEP | | | |
| | | | | | | | | | | DEDNPDHVELVERMAKMS | | | |
| | | | | | | | | | | FEGVSDNAIATTFEKEKIPS | | | |
| | | | | | | | | | | PGMAERRATEKRLASIKAR | | | |
| | | | | | | | | | | RLNGAEKPIMWRAQTVRW | | | |
| | | | | | | | | | | ILNHPAIGGFAFERVKHGK | | | |
| | | | | | | | | | | AHINVIRRDPGGKPLTPHT | | | |
| | | | | | | | | | | GILSGSKWLELQEKRSGK | | | |
| | | | | | | | | | | NLSDRKPGAEVEPTLLSG | | | |
| | | | | | | | | | | WRFLGCRICGGSMGQSQ | | | |
| | | | | | | | | | | GGRKRNGDLAEGNYMCA | | | |
| | | | | | | | | | | NPKGHGGLSVKRSELDEF | | | |
| | | | | | | | | | | VASKVWARLRTADMEDEH | | | |
| | | | | | | | | | | DQAWIAAAAERFALQHDL | | | |
| | | | | | | | | | | AGVADERREQQAHLDNVR | | | |
| | | | | | | | | | | RSIKDLQADRKAGLYVGR | | | |
| | | | | | | | | | | EELETWRSTVLQYRSYEA | | | |
| | | | | | | | | | | ECTTRLAELDEKMNGSTR | | | |
| | | | | | | | | | | VPSEWFSGEDPTAEGGIW | | | |
| | | | | | | | | | | ASWDVYERREFLSFFLDS | | | |
| | | | | | | | | | | VMVDRGRHPETKKYIPLK | | | |
| | | | | | | | | | | DRVTLKWAELLKEEDEAS | | | |
| | | | | | | | | | | EATERELAAL* | | | |
| NCBI | NA | NA | NC_ | WP_ | NA | BceI | NA | Bacillus | NA | MYPYDVPDYAGSYRPESL | 391 | 529 | INTc |
| | | | 0116 | 0002 | | NT | | cereus | | DVCIYLRKSRKDVEEERRA | | | |
| | | | | | | | | AH187 | | IEEGSSYNALERHRKRLFA | | | |
| | | | 58.1 | 8620 | | | | | | IAKAENHNIIDIFEEVASGE | | | |
| | | | | 6.1 | | | | | | SIQERPQMQQLLRKLEGN | | | |
| | | | | | | | | | | EIDGVLVIDLDRLGRGDML | | | |
| | | | | | | | | | | DAGMIDRAFRYSSTKIITPT | | | |
| | | | | | | | | | | DVYDPDDESWELVFGIKSL | | | |
| | | | | | | | | | | ISRQELKSITKRLQNGRIDS | | | |
| | | | | | | | | | | VKEGKHIGKKPPYGYLKDE | | | |
| | | | | | | | | | | NLRLYPDPEKAWIVKKIFEL | | | |
| | | | | | | | | | | MCDGKGRQMIAAELDRLG | | | |
| | | | | | | | | | | IDPPVTKRGAWDSSTITSII | | | |
| | | | | | | | | | | KNEVYTGVIVWGKFKHKK | | | |
| | | | | | | | | | | RNGKYTRHKNPQEKWIMY | | | |
| | | | | | | | | | | ENAHEPIISKELFDAANEA | | | |
| | | | | | | | | | | HSSRHKPAVITSKKLTNPL | | | |
| | | | | | | | | | | AGILKCKLCGYTMLIQTRK | | | |
| | | | | | | | | | | DRPHNYLRCNNPACKGKQ | | | |
| | | | | | | | | | | KQSVFNLVEEKLLYSLQQI | | | |
| | | | | | | | | | | VDEYQAQKVEEVEIDDSKL | | | |
| | | | | | | | | | | ISFKEKAIISKEKELKELQA | | | |
| | | | | | | | | | | QKGNLHDLLEQGIYTVEIFL | | | |
| | | | | | | | | | | ERQKNLVERITSIENDIEVL | | | |
| | | | | | | | | | | QKEIETEQIKEHNKTEFIPA | | | |
| | | | | | | | | | | LKTVIESYHKTTNIELKNQL | | | |
| | | | | | | | | | | LKTILSTVTYYRHPDWKTN | | | |
| | | | | | | | | | | EFEIQVYFKIS* | | | |
| NCBI | NA | NA | NC_ | WP_ | NA | BcyI | NA | Bacillus | NA | MYPYDVPDYAGSAVGIYIR | 392 | 487 | INTd |
| | | | 00 | 0120 | | NT | | cyto- | | VSTQEQASEGHSIESQKK | | | |
| | | | 9674. | 9542 | | | | toxicus | | KLASYCEIQGWDDYRFYIE | | | |
| | | | 1 | 9.1 | | | | NVH391- | | EGISGKNTNRPKLKLLMEH | | | |
| | | | | | | | | 98 | | IEKGKINILLVYRLDRLTRS | | | |
| | | | | | | | | | | VIDLHKLLNFLQEHGCAFK | | | |
| | | | | | | | | | | SATETYDTTTANGRMSMG | | | |
| | | | | | | | | | | IVSLLAQWETENMSERIKL | | | |
| | | | | | | | | | | NLEHKVLVEGERVGAIPYG | | | |
| | | | | | | | | | | FDLSDDEKLVKNEKSAILL | | | |
| | | | | | | | | | | DMVERVENGWSVNRIVNY | | | |
| | | | | | | | | | | LNLTNNDRNWSPNGVLRL | | | |
| | | | | | | | | | | LRNPALYGATRWNDKIAE | | | |
| | | | | | | | | | | NTHEGIISKERFNRLQQILA | | | |
| | | | | | | | | | | DRSIHHRRDVKGTYIFQGV | | | |
| | | | | | | | | | | LRCPVCDQTLSVNRFIKKR | | | |
| | | | | | | | | | | KDGTEYCGVLYRCQPCIK | | | |
| | | | | | | | | | | QNKYNLAIGEARFLKALNE | | | |
| | | | | | | | | | | YMSTVEFQTVEDEVIPKKS | | | |
| | | | | | | | | | | EREMLESQLQQIARKREK | | | |
| | | | | | | | | | | YQKAWASDLMSDDEFEKL | | | |
| | | | | | | | | | | MVETRETYDECKQKLESC | | | |
| | | | | | | | | | | EDPIKIDETYLKEIVYMFHQ | | | |
| | | | | | | | | | | TFNDLESEKQKEFISKFIRT | | | |
| | | | | | | | | | | IRYTVKEQQPIRPDKSKTG | | | |
| | | | | | | | | | | KGKQKVIITEVE FYQS* | | | |
| NCBI | NA | NA | NC_ | WP_0145 | NA | SluI | NA | Staphy- | NA | MYPYDVPDYAGSKVAIYT | 393 | 473 | INTd |
| | | | 0173 | 3323 | | NT | | lococcus | | RVSSAEQANEGYSIHEQK | | | |
| | | | 53.1 | 8.1 | | | | lug- | | KKLISYCEIHDWNEYKVFT | | | |
| | | | | | | | | dunensis | | DAGISGGSMKRPALQKLM | | | |
| | | | | | | | | N920143 | | KHLSSFDLVLVYKLDRLTR | | | |
| | | | | | | | | | | NVRDLLDMLEEFEQYNVS | | | |
| | | | | | | | | | | FKSATEVFDTTSAIGKLFIT | | | |
| | | | | | | | | | | MVGAMAEWERETIRERSL | | | |
| | | | | | | | | | | FGSRAAVREGNYIREAPF | | | |
| | | | | | | | | | | CYDNIEGKLHPNEYAKVID | | | |
| | | | | | | | | | | LIVSMFKKGISANEIARRLN | | | |
| | | | | | | | | | | SSKVHVPNKKSWNRNSLI | | | |
| | | | | | | | | | | RLMRSPVLRGHTKYGDML | | | |
| | | | | | | | | | | IENTHEPVLSEHDYNAINN | | | |
| | | | | | | | | | | AISSKTHKSKVKHHAIFRG | | | |
| | | | | | | | | | | ALVCPQCNRRLHLYAGTV | | | |
| | | | | | | | | | | KDRKGYKYDVRRYKCETC | | | |
| | | | | | | | | | | SKNKDVKNVSFNESEVEN | | | |
| | | | | | | | | | | KFVNLLKSYELNKFHIRKV | | | |
| | | | | | | | | | | EPVKKIEYDIDKINKQKINY | | | |
| | | | | | | | | | | TRSWSLGYIEDDEYFELM | | | |
| | | | | | | | | | | EEINATKKMIEEQTTENKQ | | | |
| | | | | | | | | | | SVSKEQIQSINNFILKGWE | | | |
| | | | | | | | | | | ELTIKDKEELILSTVDKIEFN | | | |
| | | | | | | | | | | FIPKDKKHK | | | |
| | | | | | | | | | | TNTLDINNIHFKFS* | | | |

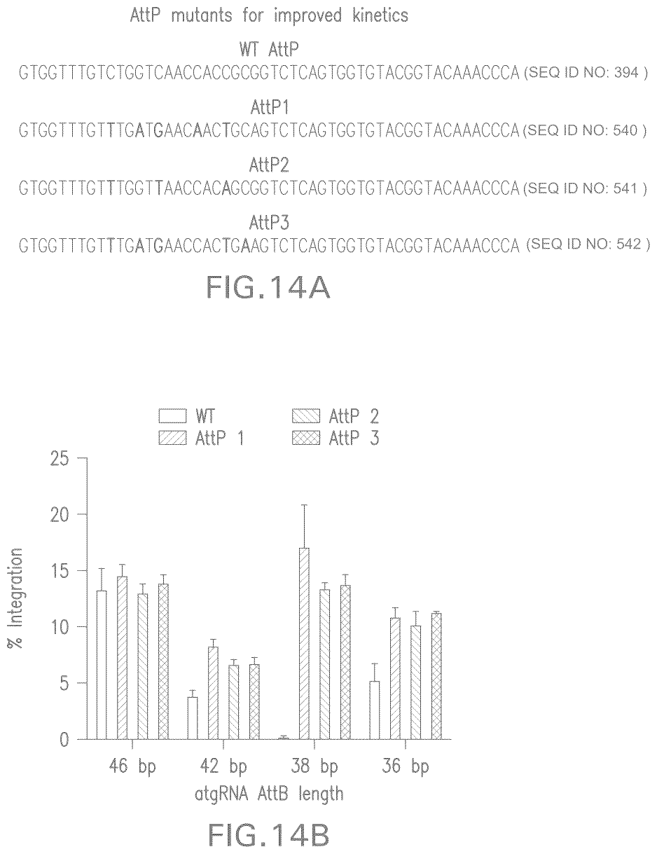

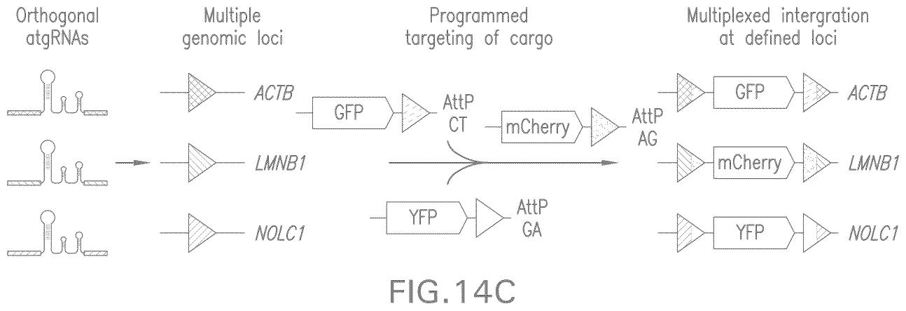

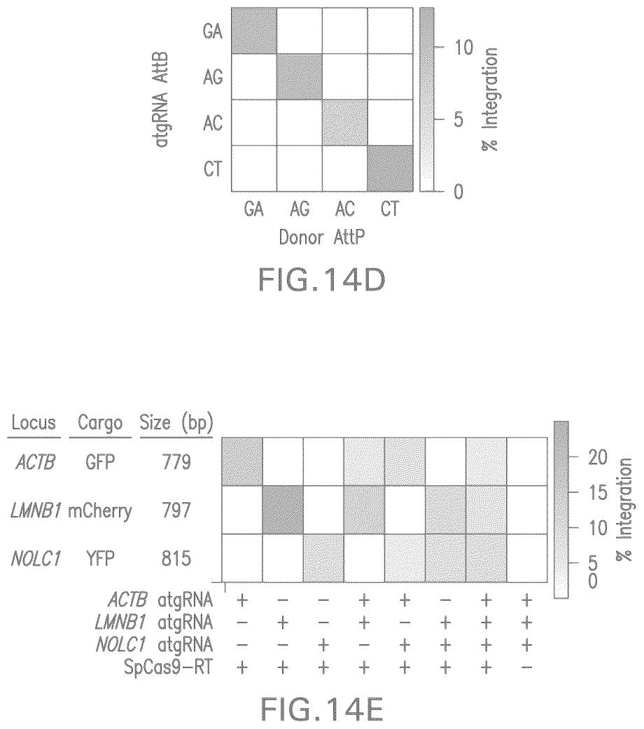

Sequences of insertion sites (i.e., recognition target sites) suitable for use in embodiments of the disclosure are presented below (Table 11). FIGS. 14A-14E shows analysis of effect of variant AttP sites on integration efficiency.

TABLE 11

| | | SEQ | Reverse | SEQ |
|---|---|---|---|---|
| | Forward | ID | Sequence | ID |
| Description | Sequence (5′-3′) | NO: | (5′-3′) | NO: |
| Bxb1_AttP. | GTGGTTTGTCTGGTC | SEQ | TGGGTTTGTACCGTA | SEQ |
| GT_original | AACCACCGCGGTCTC | ID | CACCACTGAGACCGC | ID |
| site | AGTGGTGTACGGTAC | NO: | GGTGGTTGACCAGAC | NO: |
| | AAACCCA | 394 | AAACCAC | 473 |
| Bxb1_AttP | GTGGTTTGTCTGGTC | 395 | TGGGTTTGTACCGTA | 474 |
| CG_site | AACCACCGCGcgCTC | | CACCACTGAGCGCGC | |
| | AGTGGTGTACGGTAC | | GGTGGTTGACCAGAC | |
| | AAACCCA | | AAACCAC | |
| Bxb1_AttP. | GTGGTTTGTCTGGTC | 396 | TGGGTTTGTACCGTA | 475 |
| GC_site | AACCACCGCGgcCTC | | CACCACTGAGGCCGC | |
| | AGTGGTGTACGGTAC | | GGTGGTTGACCAGAC | |
| | AAACCCA | | AAACCAC | |
| Bxb1_AttP_A | GTGGTTTGTCTGGTC | 397 | TGGGTTTGTACCGTA | 476 |
| T_site | AACCACCGCGatCTC | | CACCACTGAGATCGC | |
| | AGTGGTGTACGGTAC | | GGTGGTTGACCAGAC | |
| | AAACCCA | | AAACCAC | |
| Bxb1_AttP_T | GTGGTTTGTCTGGTC | 398 | TGGGTTTGTACCGTA | 477 |
| A_site | AACCACCGCGtaCTC | | CACCACTGAGTACGC | |
| | AGTGGTGTACGGTAC | | GGTGGTTGACCAGAC | |
| | AAACCCA | | AAACCAC | |
| Bxb1_AttP. | GTGGTTTGTCTGGTC | 399 | TGGGTTTGTACCGTA | 478 |
| GG_site | AACCACCGCGggCTC | | CACCACTGAGCCCGC | |
| | AGTGGTGTACGGTAC | | GGTGGTTGACCAGAC | |
| | AAACCCA | | AAACCAC | |
| Bxb1_AttP_T | GTGGTTTGTCTGGTC | 400 | TGGGTTTGTACCGTA | 479 |
| T_site | AACCACCGCGttCTC | | CACCACTGAGAACGC | |
| | AGTGGTGTACGGTAC | | GGTGGTTGACCAGAC | |
| | AAACCCA | | AAACCAC | |
| Bxb1_AttP | GTGGTTTGTCTGGTC | 401 | TGGGTTTGTACCGTA | 480 |
| GA_site | AACCACCGCGgaCTC | | CACCACTGAGTCCGC | |
| | AGTGGTGTACGGTAC | | GGTGGTTGACCAGAC | |
| | AAACCCA | | AAACCAC | |
| Bxb1_AttP_A | GTGGTTTGTCTGGTC | 402 | TGGGTTTGTACCGTA | 481 |
| G_site | AACCACCGCGagCTC | | CACCACTGAGCTCGC | |
| | AGTGGTGTACGGTAC | | GGTGGTTGACCAGAC | |
| | AAACCCA | | AAACCAC | |
| Bxb1_AttP | GTGGTTTGTCTGGTC | 403 | TGGGTTTGTACCGTA | 482 |
| CC_site | AACCACCGCGccCTC | | CACCACTGAGGGCGC | |
| | AGTGGTGTACGGTAC | | GGTGGTTGACCAGAC | |
| | AAACCCA | | AAACCAC | |
| Bxb1_AttP_T | GTGGTTTGTCTGGTC | 404 | TGGGTTTGTACCGTA | 483 |
| C_site | AACCACCGCGtcCTC | | CACCACTGAGGACGC | |
| | AGTGGTGTACGGTAC | | GGTGGTTGACCAGAC | |
| | AAACCCA | | AAACCAC | |
| Bxb1_AttP. | GTGGTTTGTCTGGTC | 405 | TGGGTTTGTACCGTA | 484 |
| CT_site | AACCACCGCGctCTC | | CACCACTGAGAGCGC | |
| | AGTGGTGTACGGTAC | | GGTGGTTGACCAGAC | |
| | AAACCCA | | AAACCAC | |
| Bxb1_AttP_A | GTGGTTTGTCTGGTC | 406 | TGGGTTTGTACCGTA | 485 |
| A_site | AACCACCGCGaaCTC | | CACCACTGAGTTCGC | |
| | AGTGGTGTACGGTAC | | GGTGGTTGACCAGAC | |
| | AAACCCA | | AAACCAC | |
| Bxb1_AttP. | GTGGTTTGTCTGGTC | 407 | TGGGTTTGTACCGTA | 486 |
| CA_site | AACCACCGCGcaCTC | | CACCACTGAGTGCGC | |
| | AGTGGTGTACGGTAC | | GGTGGTTGACCAGAC | |
| | AAACCCA | | AAACCAC | |
| Bxb1_AttP_A | GTGGTTTGTCTGGTC | 408 | TGGGTTTGTACCGTA | 487 |
| C site | AACCACCGCGacCTC | | CACCACTGAGGTCGC | |
| | AGTGGTGTACGGTAC | | GGTGGTTGACCAGAC | |
| | AAACCCA | | AAACCAC | |
| Bxb1_AttP_T | GTGGTTTGTCTGGTC | 409 | TGGGTTTGTACCGTA | 488 |
| G site | AACCACCGCGtgCTC | | CACCACTGAGCACGC | |
| | AGTGGTGTACGGTAC | | GGTGGTTGACCAGAC | |
| | AAACCCA | | AAACCAC | |
| Bxb1_AttB_4 | GGCCGGCTTGTCGAC | 410 | CCGGATGATCCTGAC | 489 |
| 6_GT_ | GACGGCGGTCTCCGT | | GACGGAGACCGCCGT | |
| original_site | CGTCAGGATCATCCG | | CGTCGACAAGCCGGC | |
| | G | | C | |
| Bxb1_AttB_4 | GGCCGGCTTGTCGAC | 411 | CCGGATGATCCTGAC | 490 |
| 6_AA_site | GACGGCGaaCTCCGT | | GACGGAGTTCGCCGT | |
| | CGTCAGGATCATCCG | | CGTCGACAAGCCGGC | |
| | G | | C | |
| Bxb1_AttB_4 | GGCCGGCTTGTCGAC | 412 | CCGGATGATCCTGAC | 491 |
| 6_GA_site | GACGGCGgaCTCCGT | | GACGGAGTCCGCCGT | |
| | CGTCAGGATCATCCG | | CGTCGACAAGCCGGC | |
| | G | | C | |
| Bxb1_AttB_4 | GGCCGGCTTGTCGAC | 413 | CCGGATGATCCTGAC | 492 |
| 6_CA_site | GACGGCGcaCTCCGT | | GACGGAGTGCGCCGT | |
| | CGTCAGGATCATCCG | | CGTCGACAAGCCGGC | |
| | G | | C | |
| Bxb1_AttB_4 | GGCCGGCTTGTCGAC | 414 | CCGGATGATCCTGAC | 493 |
| 6_TA_site | GACGGCGtaCTCCGT | | GACGGAGTACGCCGT | |
| | CGTCAGGATCATCCG | | CGTCGACAAGCCGGC | |
| | G | | C | |
| Bxb1_AttB_4 | GGCCGGCTTGTCGAC | 415 | CCGGATGATCCTGAC | 494 |
| 6_AG_site | GACGGCGagCTCCGT | | GACGGAGCTCGCCGT | |
| | CGTCAGGATCATCCG | | CGTCGACAAGCCGGC | |
| | G | | C | |
| Bxb1_AttB_4 | GGCCGGCTTGTCGAC | 416 | CCGGATGATCCTGAC | 495 |
| 6_GG_site | GACGGCGggCTCCGT | | GACGGAGCCCGCCGT | |
| | CGTCAGGATCATCCG | | CGTCGACAAGCCGGC | |
| | G | | C | |
| Bxb1_AttB_4 | GGCCGGCTTGTCGAC | 417 | CCGGATGATCCTGAC | 496 |
| 6_CG_site | GACGGCGcgCTCCGT | | GACGGAGCGCGCCGT | |
| | CGTCAGGATCATCCG | | CGTCGACAAGCCGGC | |
| | G | | C | |
| Bxb1_AttB_4 | GGCCGGCTTGTCGAC | 418 | CCGGATGATCCTGAC | 497 |
| 6_TG_site | GACGGCGtgCTCCGT | | GACGGAGCACGCCGT | |
| | CGTCAGGATCATCCG | | CGTCGACAAGCCGGC | |
| | G | | C | |
| Bxb1_AttB_4 | GGCCGGCTTGTCGAC | 419 | CCGGATGATCCTGAC | 498 |
| 6_AC_site | GACGGCGacCTCCGT | | GACGGAGGTCGCCGT | |
| | CGTCAGGATCATCCG | | CGTCGACAAGCCGGC | |
| | G | | C | |
| Bxb1_AttB_4 | GGCCGGCTTGTCGAC | 420 | CCGGATGATCCTGAC | 499 |
| 6_GC_site | GACGGCGgcCTCCGT | | GACGGAGGCCGCCGT | |
| | CGTCAGGATCATCCG | | CGTCGACAAGCCGGC | |
| | G | | C | |
| Bxb1_AttB_4 | GGCCGGCTTGTCGAC | 421 | CCGGATGATCCTGAC | 500 |
| 6_CC_site | GACGGCGccCTCCGT | | GACGGAGGGCGCCGT | |
| | CGTCAGGATCATCCG | | CGTCGACAAGCCGGC | |
| | G | | C | |
| Bxb1_AttB_4 | GGCCGGCTTGTCGAC | 422 | CCGGATGATCCTGAC | 501 |
| 6_TC_site | GACGGCGtcCTCCGT | | GACGGAGGACGCCGT | |
| | CGTCAGGATCATCCG | | CGTCGACAAGCCGGC | |
| | G | | C | |
| Bxb1_AttB_4 | GGCCGGCTTGTCGAC | 423 | CCGGATGATCCTGAC | 502 |
| 6_AT_site | GACGGCGatCTCCGT | | GACGGAGATCGCCGT | |
| | CGTCAGGATCATCCG | | CGTCGACAAGCCGGC | |
| | G | | C | |
| Bxb1_AttB_4 | GGCCGGCTTGTCGAC | 424 | CCGGATGATCCTGAC | 503 |
| 6_CT_site | GACGGCGctCTCCGT | | GACGGAGAGCGCCGT | |
| | CGTCAGGATCATCCG | | CGTCGACAAGCCGGC | |
| | G | | C | |
| Bxb1_AttB_4 | GGCCGGCTTGTCGAC | 425 | CCGGATGATCCTGAC | 504 |
| 6_TT_site | GACGGCGttCTCCGT | | GACGGAGAACGCCGT | |
| | CGTCAGGATCATCCG | | CGTCGACAAGCCGGC | |
| | G | | C | |
| Bxb1_AttB_3 | GGCTTGTCGACGACG | 426 | ATGATCCTGACGACG | 505 |
| 8_GT_site | GCGGTCTCCGTCGTC | | GAGACCGCCGTCGTC | |
| | AGGATCAT | | GACAAGCC | |
| Bxb1_AttB_3 | GGCTTGTCGACGACG | 427 | ATGATCCTGACGACG | 506 |
| 8_AA_site | GCGaaCTCCGTCGTC | | GAGTTCGCCGTCGTC | |
| | AGGATCAT | | GACAAGCC | |
| Bxb1_AttB_3 | GGCTTGTCGACGACG | 428 | ATGATCCTGACGACG | 507 |
| 8_GA_site | GCGgaCTCCGTCGTC | | GAGTCCGCCGTCGTC | |
| | AGGATCAT | | GACAAGCC | |
| Bxb1_AttB_3 | GGCTTGTCGACGACG | 429 | ATGATCCTGACGACG | 508 |
| 8_CA_site | GCGcaCTCCGTCGTC | | GAGTGCGCCGTCGTC | |
| | AGGATCAT | | GACAAGCC | |
| Bxb1_AttB_3 | GGCTTGTCGACGACG | 430 | ATGATCCTGACGACG | 509 |
| 8_TA_site | GCGtaCTCCGTCGTC | | GAGTACGCCGTCGTC | |
| | AGGATCAT | | GACAAGCC | |
| Bxb1_AttB_3 | GGCTTGTCGACGACG | 431 | ATGATCCTGACGACG | 510 |
| 8_AG_site | GCGagCTCCGTCGTC | | GAGCTCGCCGTCGTC | |
| | AGGATCAT | | GACAAGCC | |
| Bxb1_AttB_3 | GGCTTGTCGACGACG | 432 | ATGATCCTGACGACG | 511 |
| 8_GG_site | GCGggCTCCGTCGTC | | GAGCCCGCCGTCGTC | |
| | AGGATCAT | | GACAAGCC | |
| Bxb1_AttB_3 | GGCTTGTCGACGACG | 433 | ATGATCCTGACGACG | 512 |
| 8_CG_site | GCGcgCTCCGTCGTC | | GAGCGCGCCGTCGTC | |
| | AGGATCAT | | GACAAGCC | |
| Bxb1_AttB_3 | GGCTTGTCGACGACG | 434 | ATGATCCTGACGACG | 513 |
| 8_TG_site | GCGtgCTCCGTCGTC | | GAGCACGCCGTCGTC | |
| | AGGATCAT | | GACAAGCC | |
| Bxb1_AttB_3 | GGCTTGTCGACGACG | 435 | ATGATCCTGACGACG | 514 |
| 8_AC_site | GCGacCTCCGTCGTC | | GAGGTCGCCGTCGTC | |
| | AGGATCAT | | GACAAGCC | |
| Bxb1_AttB_3 | GGCTTGTCGACGACG | 436 | ATGATCCTGACGACG | 515 |
| 8_GC_site | GCGgcCTCCGTCGTC | | GAGGCCGCCGTCGTC | |
| | AGGATCAT | | GACAAGCC | |
| Bxb1_AttB_3 | GGCTTGTCGACGACG | 437 | ATGATCCTGACGACG | 516 |
| 8_CC_site | GCGccCTCCGTCGTC | | GAGGGCGCCGTCGTC | |
| | AGGATCAT | | GACAAGCC | |
| Bxb1_AttB_3 | GGCTTGTCGACGACG | 438 | ATGATCCTGACGACG | 517 |
| 8_TC_site | GCGtcCTCCGTCGTC | | GAGGACGCCGTCGTC | |
| | AGGATCAT | | GACAAGCC | |
| Bxb1_AttB_3 | GGCTTGTCGACGACG | 439 | ATGATCCTGACGACG | 518 |
| 8_AT_site | GCGatCTCCGTCGTC | | GAGATCGCCGTCGTC | |
| | AGGATCAT | | GACAAGCC | |
| Bxb1_AttB_3 | GGCTTGTCGACGACG | 440 | ATGATCCTGACGACG | 519 |
| 8_CT_site | GCGctCTCCGTCGTC | | GAGAGCGCCGTCGTC | |
| | AGGATCAT | | GACAAGCC | |
| Bxb1_AttB_3 | GGCTTGTCGACGACG | 441 | ATGATCCTGACGACG | 520 |
| 8_TT_site | GCGttCTCCGTCGTC | | GAGAACGCCGTCGTC | |
| | AGGATCAT | | GACAAGCC | |
| Cre Lox 66 | TACCGTTCGTATAAT | 442 | ATAACTTCGTATAGC | 521 |
| site | GTATGCTATACGAAG | | ATACATTATACGAAC | |
| | TTAT | | GGTA | |
| Cre Lox 71 | ATAACTTCGTATAAT | 443 | TACCGTTCGTATAGC | 522 |
| site | GTATGCTATACGAAC | | ATACATTATACGAAG | |
| | GGTA | | TTAT | |
| TP901-1 | TTTACCTTGATTGAG | 444 | CACAATTAACATCTC | 523 |
| minimal AttB | ATGTTAATTGTG | | AATCAAGGTAAA | |
| site | | | | |
| TP901-1 | GCGAGTTTTTATTTC | 445 | AAAGGAGTTTTTTAG | 524 |
| minimal AttP | GTTTATTTCAATTAA | | TTACCTTAATTGAAA | |
| site | GGTAACTAAAAAACT | | TAAACGAAATAAAAA | |
| | CCTTT | | CTCGC | |
| PhiBT1 | CTGGATCATCTGGAT | 446 | CAGGTTTTTGACGAA | 525 |
| minimal AttB | CACTTTCGTCAAAAA | | AGTGATCCAGATGAT | |
| site | CCTG | | CCAG | |
| PhiBT1 | TTCGGGTGCTGGGTT | 447 | TGGTGCTGAGTAGTT | 526 |
| minimal AttP | GTTGTCTCTGGACAG | | TCCCATGGATCACTG | |
| site | TGATCCATGGGAAAC | | TCCAGAGACAACAAC | |
| | TACTCAGCACCA | | CCAGCACCCGAA | |
| Bacillus_ | gatatggggaagtga | 448 | ggtactgtggcggtt | 527 |
| cereus_AH1 | atcagtacaaccgcc | | gtactgattcacttc | |
| 87_Int30_38 | acagtacc | | cccatatc | |
| bp_Att | | | | |
| Staphylococcus_ | tgggtggtacaggtg | 449 | cataaatggtacaac | 528 |
| lugdunensis_ | ccacattagttgtac | | taatgtggcacctgt | |
| N920143 | catttatg | | accaccca | |
| Int1 | | | | |
| 2_38bp_Att | | | | |
| Bacillus_ | gttgtttttccagat | 450 | cttatatttacagga | 529 |
| cytotoxicus_ | ccagttggtcctgta | | ccaactggatctgga | |
| NVH_391- | aatataag | | aaaacaac | |
| 98_Int13_38 | | | | |
| bp_Att | | | | |
| Bacillus_ | tggggaagtgaatca | 451 | ctgtggcggttgtac | 454 |
| cereus_ | gtacaaccgccacag | | tgattcacttcccca | |
| AH187_ | | | | |
| Int30_ | | | | |
| Att30 | | | | |
| Bacillus_ | ggggaagtgaatcag | 452 | tgtggcggttgtact | 455 |
| cereus_ | tacaaccgccaca | | gattcacttcccc | |
| AH187_ | | | | |
| Int30_ | | | | |
| Att28 | | | | |
| Bacillus_ | gggaagtgaatcagt | 453 | gtggcggttgtactg | 456 |
| cereus_ | acaaccgccac | | attcacttccc | |
| AH187_ | | | | |
| Int30_ | | | | |
| Att26 | | | | |
| Bacillus_ | ctgtggcggttgtac | 454 | tggggaagtgaatca | 451 |
| cereus_ | tgattcacttcccca | | gtacaaccgccacag | |
| AH187_ | | | | |
| Int30_ | | | | |
| Att_ | | | | |
| rc_ | | | | |
| 30 | | | | |
| Bacillus_ | tgtggcggttgtact | 455 | ggggaagtgaatcag | 452 |
| cereus_ | gattcacttcccc | | tacaaccgccaca | |
| AH187_ | | | | |
| Int30_ | | | | |
| Att_ | | | | |
| rc_ | | | | |
| 28 | | | | |
| Bacillus_ | gtggcggttgtactg | 456 | gggaagtgaatcagt | 453 |
| cereus_ | attcacttccc | | acaaccgccac | |
| AH187_ | | | | |
| Int30_ | | | | |
| Att_ | | | | |
| rc_ | | | | |
| 26 | | | | |
| Bacillus_ | tttttccagatccag | 457 | tatttacaggaccaa | 460 |
| cytotoxicus_ | ttggtcctgtaaata | | ctggatctggaaaaa | |
| NVH_ | | | | |
| 391-98_ | | | | |
| Int13_ | | | | |
| Att_ | | | | |
| 30 | | | | |
| Bacillus_ | ttttccagatccagt | 458 | atttacaggaccaac | 461 |
| cytotoxicus_ | tggtcctgtaaat | | tggatctggaaaa | |
| NVH_ | | | | |
| 391-98_ | | | | |
| Int13_ | | | | |
| Att28 | | | | |
| Bacillus_ | tttccagatccagtt | 459 | tttacaggaccaact | 462 |
| cytotoxicus_ | ggtcctgtaaa | | ggatctggaaa | |
| NVH_ | | | | |
| 391-98_ | | | | |
| Int13_ | | | | |
| Att26 | | | | |
| Bacillus_ | tatttacaggaccaa | 460 | tttttccagatccag | 457 |
| cytotoxicus_ | ctggatctggaaaaa | | ttggtcctgtaaata | |
| NVH_ | | | | |
| 391-98_ | | | | |
| Int13_ | | | | |
| Att_ | | | | |
| rc_ | | | | |
| 30 | | | | |
| Bacillus_ | atttacaggaccaac | 461 | ttttccagatccagt | 458 |
| cytotoxicus_ | tggatctggaaaa | | tggtcctgtaaat | |
| NVH_ | | | | |
| 391-98_ | | | | |
| Int13_ | | | | |
| Attrc_ | | | | |
| 28 | | | | |
| Bacillus_ | tttacaggaccaact | 462 | tttccagatccagtt | 459 |
| cytotoxicus_ | ggatctggaaa | | ggtcctgtaaa | |
| NVH_ | | | | |
| 391-98_ | | | | |
| Int13_ | | | | |
| Att_ | | | | |
| rc_ | | | | |
| 26 | | | | |
| N680429_56 | CATTATATGTTTTTA | 1463 | cattatatgttctta | 530 |
| 0_31_50bp | CAATCCGGGCCGCCA | | cagtatggcggcccg | |
| | TACTGTAAGAACATA | | gattgtaaaaacata | |
| | TAATG | | taatg | |
| N191607_8 | CGTTATAGGGTATTG | 1464 | cgttatagggtatta | 531 |
| 101_50bp | CAGTACCGACCGCCA | | cagtatggcggtcgg | |
| | TACTGTAATACCCTA | | tactgcaatacccta | |
| | TAACG | | taacg | |
| N674992_1_ | TGTATCATTTTCATA | 1465 | tgtatcattttcata | 532 |
| 1308_50bp | TAGTGTGCAGGTGCT | | tagttagcacctgca | |
| | AACTATATGAAAATG | | cactatatgaaaatg | |
| | ATACA | | ataca | |
| N684613_54 | TGTCTACTATGTCTT | 466 | tgtctactatctgta | 533 |
| _96_50bp | TATGCCACATGTGTC | | tatgcgacacatgtg | |
| | GCATATACAGATAGT | | gcataaagacatagt | |
| | AGACA | | agaca | |
| N252616_12 | AATGAGGTCAGACGC | 467 | catcgaccctgacgc | 534 |
| 1_74_50bp | ATGGAGCGCCGCCTC | | atgcggaggcggcgc | |
| | CGCATGCGTCAGGGT | | tccatgcgtctgacc | |
| | CGATG | | tcatt | |
| N683040_22 | GTTAGTACCCAAATG | 468 | gttagtacccaaatg | 535 |
| 2_19_50bp | ATAAAAGGATGACCT | | acaaaaggtcatcct | |
| | TTTGTCATTTGGGTA | | tttatcatttgggta | |
| | CTAAC | | ctaac | |
| N687537_17 | GTTTATAAAACCGAT | 469 | cttattaaaacccgt | 536 |
| 3_59_50bp | GCCGCTTTGACAGAA | | tccgcttctgtcaaa | |
| | GCGGAACGGGTTTTA | | gcggcatcggtttta | |
| | ATAAG | | taaac | |
| N183629_47_ | GGCCGCGAGGTCGTG | 470 | ggcgtgatggtcgtg | 537 |
| 40_50b p | TTCGTCGTCATGTTG | | aacctcaacatgacg | |
| | AGGTTCACGACCATC | | acgaacacgacctcg | |
| | ACGCC | | cggcc | |
| N191533_22 | TATAAACTGATATAA | 1471 | tctacatcttgaata | 538 |
| 4_76_50bp | TTCAAAGTTATAACT | | tatcaagttataact | |
| | TGATATATTCAAGAT | | ttgaattatatcagt | |
| | GTAGA | | ttata | |
| N682356_18 | TATTATATCTAAAAG | 472 | aattatatctaaaag | 539 |
| 8_20_50 bp | CAGTATGGCGGAGCT | | cactaagctccgcca | |
| | TAGTGCTTTTAGATA | | tactgcttttagata | |
| | TAATT | | taata | |

In some embodiments, the integration enzyme is selected from one of the about 27,000 Serine integrases described in International Patent Publication No. WO 2023/070031A2, such as SEQ ID NOs: 1-16 and 163-1162 and 3166-3175 of WO2023/070031 (which PCT publication is hereby incorporated by reference in its entirety).

Other suitable integrase (such as the eLSR described herein) suitable for use with the invention described herein includes SEQ ID NOs: 85-158 of WO2023/177424 (which PCT publication is hereby incorporated by reference in its entirety).

### 6.9. Engineered Integration Enzymes

This disclosure features integration enzymes (also referred to integrases or eLSR) engineered such that upon being introduced into a cell, the integration enzyme has increased fidelity and stability (e.g., half-life) compared to a control integration enzyme not engineered to have increased stability (cLSR). An increased fidelity entails substantially maintaining or enhancing integration activity at a pair of cognate integration recognition sites, and substantially decreasing off-target integration activity at a pair of off-target integration recognition sites, when compared to a corresponding large serine integrase without said one or more substitutions (cLSR). An increase in stability includes an increase in half-life and a reduction in protein degradation rates, thereby extending the capacity of the integration enzyme to mediate integration.

Without wishing to be bound by theory, it is believed that off-target integration of a donor DNA by integrase is partially influenced by the sequence similarity between the cognate integration recognition sequences of the integrase (such as the attB and attP sites) and the non-cognate integration recognition sequences, such as the off-target integration recognition sites CAS031 attB sequence and CAS031 attP sequence; and the CAS421 attB sequence and CAS421 attP sequence (see FIG. 59). It is further believed that such sequence similarity tends to be concentrated in the integrase recombinase domain (RD) binding/interacting region, and to a less extent in the ZD domain. Hence mutating certain residues in the ZD could reduce off-target integration.

Also, without wishing to be bound by theory, there are two main pathways for protein degradation: proteolysis by ubiquitin proteosome system (UPS) and autophagy. Under the UPS system, proteins can be ubiquitinated on lysine residues, thereby leading to degradation. Proteins can also have specific domains (degrons) that can be ubiquitinated, which also leads to protein degradation. For example, in the UPS, Degrons (also referred to as degradation signals) are recognized and polyubiquitinated, targeting the protein for degradation. Degrons can be present at both the N-terminus and/or C-terminus of a protein. Polyubiquitinated protein is recognized by receptor subunits of the 26S proteosome (Rpn10 (yeast)/p54 (drosophila), Rpn13 and Rpn1).

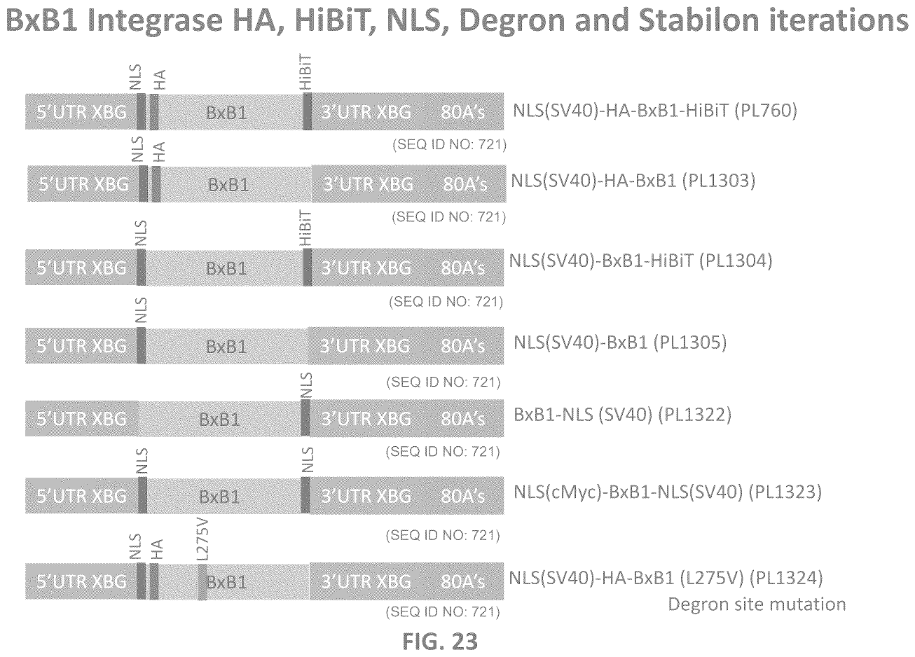

In some embodiments, the engineered integration enzymes is selected from the engineered integration enzymes described in FIGS. 23, 25, 28, 30B, 32A, 33, 35, and 40-42. In some embodiments, the engineered integration enzymes is selected from the engineered integration enzymes described in Table 26. In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence listed in Table 26. In some embodiments, the engineered integration enzyme is encoded by a nucleic acid sequence that is at least 80% (e.g., 80%, 85%, 90%, 91%, 92%, 93%, 94%, 95%, 96%, 97%, 99%, or 100%) identical to a nucleic acid sequence listed in Table 26.

### 6.9.1. Engineered Integration Enzyme with a High Fidelity Mutation

This disclosure features integration enzymes engineered to include a so-called high fidelity mutation (or HiFi mutation) that maintain or enhance integration activity at a pair of cognate integration recognition sites, and substantially decrease off-target integration activity at a pair of off-target integration recognition sites, when compared to a corresponding large serine integrase without said one or more substitutions (cLSR). In other words, the integration enzymes (e.g., engineered large serine integrase or eLSR) are engineered such that upon being introduced into a cell, the integration enzyme/eLSR has increased fidelity/specificity towards the cognate integration recognition sequences/sites at the target integration sequence, over off-target integration recognition sequences/sites at off-target integration sequences. The engineered large serine integrase (eLSR) described herein comprises one or more substitutions (e.g., substitutions in a zinc ribbon domain (ZD)) that substantially maintain or enhance integration activity at a pair of cognate integration recognition sites, and substantially decrease off-target integration activity at a pair of off-target integration recognition sites, when compared to a corresponding large serine integrase without said one or more substitutions (cLSR).

In certain embodiments, the one or more substitutions are in a zinc ribbon domain (ZD) of the cLSR.

In certain embodiments, the cLSR comprises an amino acid sequence that is at least 80% identical to any one of (a) SEQ ID NOs: 378-393; (b) SEQ ID NOs: 85-158 of WO2023/177424 (incorporated herein by reference); and (c) SEQ ID NOs: 1-16 and 163-1162 and 3166-3175 of WO2023/070031 (incorporated herein by reference).

Although many of the examples herein are based on one representative eLSR—the Bxb1 LSR, the disclosure herein is not so limited, since other LSRs with similar sequence or structure can be readily engineered similarly as Bxb1, based on identifying corresponding amino acids at the positions in Bxb1, e.g., residues 315, 318, 319, and 329 of Bxb1 (such as SEQ ID NO: 388), to make similar mutations to enhance on-target integration at the cognate integration recognition sequences, and/or to reduce or even eliminate off-target integration at the non-cognate integration recognition sequences.

In certain embodiments, the corresponding residues in the ZD domain can be identified through sequence alignment between Bxb1 (such as SEQ ID NO: 388) and any other LSRs.

In certain embodiments, the corresponding residues in the ZD domain can be identified through structural similarity between Bxb1 (such as SEQ ID NO: 388) and any other LSRs. This is particularly helpful when sequence identity between the LSRs is low, yet the overall three-dimensional foldings are similar or substantially the same.

In certain embodiments, the cLSR is a BxB1 polypeptide, SsuINT, SssINT, SscINT, Ssc2INT, SsdINT, SmcINT, UhmINT, SacINT, RsaINT, Rsa2INT, Bxb1, Tp91NT, Bt1INT, BceINT, BcyINT, SluINT, or a functional fragment/variant thereof.

In certain embodiments, the cLSR is a BxB1 polypeptide or a functional fragment/variant thereof.

In certain embodiments, the cLSR comprises an amino acid sequence having at least 80% sequence identity to SEQ ID NO: 388. In certain embodiments, the cLSR has an amino acid sequence of SEQ ID NO: 388.

In certain embodiments, the pair of cognate integration recognition sites are: an attB sequence and an attP sequence; or a modified AttB sequence and a modified AttP sequence.

In certain embodiments, the pair of off-target integration recognition sites are CAS031 attB sequence and CAS031 attP sequence; or CAS421 attB sequence and CAS421 attP sequence. See FIG. 59.

In certain embodiments, at least one of the pair of cognate integration recognition sites is integrated into a mammalian cell genome at a target DNA sequence. In certain embodiments, the at least one of the pair of cognate integration recognition sites is integrated into the mammalian cell genome at the target DNA sequence by: (1) programmable addition through site-specific targeting elements (PASTE) using a n attachment site-containing guide RNA (atgRNA) and a gene editor polypeptide; (2) homology directed repair (HDR), such as short-fragment homologous recombination (SFHR); (3) ligation-assisted homologous recombination (LAHR); (4) ligation-assisted Replacer (e.g., Replacer 1 or Replacer 2) editing; or, (5) non-homologous end joining (NHEJ), such as homology-independent targeted insertion (HITI).

Programmable Addition via Site-specific Targeting Elements (PASTE) herein refers to a gene editing tool that uses a CRISPR-Cas9 nickase fused to both a reverse transcriptase and serine integrase for targeted genomic recruitment and integration of desired payloads (e.g., “beacon” or integration recognition sequences/sites for the LSR of the invention). This insertion can be done without introducing double-stranded breaks by adding one DNA strand first via a fused reverse transcriptase, then its complementary strand. See Ionnidi et al.; doi: 10.1101/2021.11.01.466786 (the entirety of Ionnidi et al. is incorporated by reference). Non-limiting examples of PASTE are also described in U.S. Pat. No. 11,572,556 and PCT Publication Nos. WO 2022/087235A and WO 2023/077148A1, each of which are hereby incorporated by reference in their entireties.

Homology-directed repair (HDR) is a process where a DNA double-strand break (DSB) is repaired by homologous recombination using a DNA template (e.g., a DNA template comprising the beacon or integration recognition sequences/sites flanked by two homologous arm sequences for integrating the beacon into a target site at the DSB region.

Small fragment homologous replacement (SFHR) is a type of HDR. It is characterized by the use of small exogenous DNA fragments (SDFs, up to 1 kb, such as the beacon or integration recognition sequences/sites for the LSR of the invention). Small DNA fragments (SDFs) are used in SFHR to modify endogenous genomic DNA (e.g., insertion of the beacon at the endogenous genomic DNA sequence as the target integration locus). SFHR involves an interaction between the SDF sequence (e.g., wild-type SDF sequence) and the target endogenous sequence (e.g., mutated endogenous sequence).

Non-homologous end joining (NHEJ) is a pathway that repairs double-strand breaks in DNA. It is called “non-homologous” because the break ends are directly ligated without the need for a homologous template, in contrast to homology directed repair (HDR), which requires a homologous sequence to guide repair. In this case, the beacon or the integration recognition sequences/sites for the LSR of the invention can be provided as donor DNA that can be integrated into the target genomic location around the DSB via NHEJ.

Ligation-Assisted Homologous Recombination” (LAHR) was discovered in association with Cas12a. Unlike the blunt-ended DSB generated by Cas9, Cas12a generates sticky-ended DSB that can aid precise genome editing. A short double-stranded DNA (dsDNA) repair template (such as one comprising the beacon or the integration recognition sequences/sites for the LSR of the invention) containing a sticky end that matched one of the Cas12a-generated DSB ends and a homologous arm sharing homology with the genomic region adjacent to the other end of the DSB enables precise repair of the DSB and introduced a desired nucleotide substitution. This strategy is termed “Ligation-Assisted Homologous Recombination” (LAHR).

Ligation-assisted Replacer editing herein refers to a nucleic acid editing process. It includes ligation of an integrating nucleic acid (such as the beacon or the integration recognition sequences/sites for the LSR of the invention) to a target nucleic acid and replacement of a portion of the target nucleic acid with the integrating nucleic acid. In some embodiments, the method ligation-assisted Replacer editing refers to what is described in International Patent Publication No. WO2023086834A1, which is herein incorporated by reference in its entirety.

In certain embodiments, the one or more substitutions are at a position corresponding to residue 315, residue 318, residue 319, and/or residue 320 of SEQ ID NO: 388.

In certain embodiments, the one or more substitutions are at a position corresponding to residue 315 of SEQ ID NO: 388, residue 318 of SEQ ID NO: 388, residue 319 of SEQ ID NO: 388, or residue 320 of SEQ ID NO: 388. In certain embodiments, the one or more substitutions are at a position corresponding to residue 315 of SEQ ID NO: 388. In certain embodiments, the one or more substitutions are at a position corresponding to residue 318 of SEQ ID NO: 388. In certain embodiments, the one or more substitutions are at a position corresponding to residue 319 of SEQ ID NO: 388. In certain embodiments, the one or more substitutions are at a position corresponding to residue 320 of SEQ ID NO: 388.

In certain embodiments, the one or more substitutions comprise a single residue substitution. In certain embodiments, the single residue substitution is at a position corresponding to residue 315 of SEQ ID NO: 388. In certain embodiments, the single residue substitution is at a position corresponding to residue 318 of SEQ ID NO: 388. In certain embodiments, the single residue substitution is at a position corresponding to residue 319 of SEQ ID NO: 388. In certain embodiments, the single residue substitution is at a position corresponding to residue 320 of SEQ ID NO: 388.

In certain embodiments, the single residue substitution is to any of the other 19 proteinogenic amino acids different from the original amino acid at such positions. For example, if the single residue substitution is at a position corresponding to residue K320 of SEQ ID NO: 388, the substituted residue can be any one of A, C, D, E, F, G, H, I, L, M, N, P, Q, R, S, T, V, W, and Y, such as E (i.e., K320E).

In certain embodiments, the single residue substitution is to an amino acid in a different category as the original amino acid. In this regard, the 20 proteinogenic amino acids can be grouped into different categories, including basic amino acids comprising K, R, and H; acidic amino acids comprising D and E; polar amino acids comprising S, T, Q, and N; non-polar or aliphatic/hydrophobic amino acids comprising: A, V, I, L, and M; aromatic amino acids comprising: F, Y, and W; and special amino acids comprising P, G, and C.

For example, residue K320 can be substituted by any one amino acid from the acidic amino acids (such as E or D), by any one amino acid from the polar amino acids; by any one amino acid from the non-polar or aliphatic/hydrophobic amino acids; by any one amino acid from the aromatic amino acids; or by any one amino acid from the special amino acids.

In certain embodiments, the single residue substitution is at a position corresponding to residue A315 of SEQ ID NO: 388, to a basic amino acid, to an acidic amino acid, to a polar amino acid; to a non-polar or aliphatic/hydrophobic amino acid; to an aromatic amino acid; or to a special amino acid. In certain embodiments, the single residue substitution is at a position corresponding to residue A315 of SEQ ID NO: 388, to an acidic amino acid, such as D or E. In certain embodiments, the single residue substitution is at a position corresponding to residue A315 of SEQ ID NO: 388, to a basic amino acid, such as R or K.

In certain embodiments, the one or more substitutions comprise or consist of a substitution to D (Asp), E (Glu) or R (Arg) at the position corresponding to residue 315 of SEQ ID NO: 388. In certain embodiments, the one or more substitutions comprise or consist of a substitution to R (Arg) at the position corresponding to residue 315 of SEQ ID NO: 388.

In certain embodiments, the single residue substitution is at a position corresponding to residue A318 of SEQ ID NO: 388, to a basic amino acid, to an acidic amino acid, to a polar amino acid; to a non-polar or aliphatic/hydrophobic amino acid; to an aromatic amino acid; or to a special amino acid. In certain embodiments, the single residue substitution is at a position corresponding to residue A318 of SEQ ID NO: 388, to an acidic amino acid, such as D or E.

In certain embodiments, the one or more substitutions comprise or consist of a substitution to E (Glu) at the position corresponding to residue 318 of SEQ ID NO: 388.

In certain embodiments, the single residue substitution is at a position corresponding to residue A319 of SEQ ID NO: 388, to a basic amino acid, to an acidic amino acid, to a polar amino acid; to a non-polar or aliphatic/hydrophobic amino acid; to an aromatic amino acid; or to a special amino acid. In certain embodiments, the single residue substitution is at a position corresponding to residue A319 of SEQ ID NO: 388, to a polar amino acid, such as Q or N.

In certain embodiments, the one or more substitutions comprise or consist of a substitution to Q (Gln) at the position corresponding to residue 319 of SEQ ID NO: 388.

In certain embodiments, the single residue substitution is at a position corresponding to residue A320 of SEQ ID NO: 388, to a basic amino acid, to an acidic amino acid, to a polar amino acid; to a non-polar or aliphatic/hydrophobic amino acid; to an aromatic amino acid; or to a special amino acid. In certain embodiments, the single residue substitution is at a position corresponding to residue A320 of SEQ ID NO: 388, to an acidic amino acid, such as D or E. In certain embodiments, the single residue substitution is at a position corresponding to residue A320 of SEQ ID NO: 388, to a basic amino acid, such as R or K. In certain embodiments, the single residue substitution is at a position corresponding to residue A320 of SEQ ID NO: 388, to a polar amino acid, such as Q or N. In certain embodiments, the single residue substitution is at a position corresponding to residue A320 of SEQ ID NO: 388, to an aromatic amino acid, such as F, W, or Y.

In certain embodiments, the one or more substitutions comprise or consist of a substitution to E (Glu), Q (Gln), or R (Arg) at the position corresponding to residue 320 of SEQ ID NO: 388. In certain embodiments, the one or more substitutions comprise or consist of a substitution to E (Glu) at the position corresponding to residue 320 of SEQ ID NO: 388. In certain embodiments, the one or more substitutions comprise or consist of a substitution to R (Arg) at the position corresponding to residue 320 of SEQ ID NO: 388.

In certain embodiments, the one or more substitutions do not include A315F, A315G, A315H, A3151, A315M, A315N, A315S, A315T, A315W, A315Y, G3181, G318K, G318R, and G318W.

In certain embodiments, the one or more substitutions comprise or consist of two of the four positions corresponding to residue 315 of SEQ ID NO: 388, residue 318 of SEQ ID NO: 388, residue 319 of SEQ ID NO: 388, and residue 320 of SEQ ID NO: 388. For example, the two positions can be 315 and 318, 315 and 319, 315 and 320, 318 and 319, 318 and 320, or 319 and 320. In each instant or combination, the substitutions at each one of the two positions can be independently as described above for single substitutions at each position.

In certain embodiments, the one or more substitutions comprise or consist of the position corresponding to residue 315 of SEQ ID NO: 388, and the position corresponding to residue 320 of SEQ ID NO: 388.

In certain embodiments, the one or more substitutions comprise or consist of a substitution to D (Asp), E (Glu) or R (Arg) at the position corresponding to residue 315 of SEQ ID NO: 388; and a substitution to E (Glu), Q (Gln), or R (Arg) at the position corresponding to residue 320 of SEQ ID NO: 388.

In certain embodiments, the one or more substitutions comprise or consist of a substitution to D at the position corresponding to residue 315 of SEQ ID NO: 388; and a substitution to E at the position corresponding to residue 320 of SEQ ID NO: 388.

In certain embodiments, the one or more substitutions comprise or consist of a substitution to E at the position corresponding to residue 315 of SEQ ID NO: 388; and a substitution to E at the position corresponding to residue 320 of SEQ ID NO: 388.

In certain embodiments, the one or more substitutions comprise or consist of a substitution to R at the position corresponding to residue 315 of SEQ ID NO: 388; and a substitution to E at the position corresponding to residue 320 of SEQ ID NO: 388.

In certain embodiments, the one or more substitutions comprise or consist of a substitution to D at the position corresponding to residue 315 of SEQ ID NO: 388; and a substitution to R at the position corresponding to residue 320 of SEQ ID NO: 388.

In certain embodiments, the one or more substitutions comprise or consist of a substitution to E at the position corresponding to residue 315 of SEQ ID NO: 388; and a substitution to R at the position corresponding to residue 320 of SEQ ID NO: 388.

In certain embodiments, the one or more substitutions comprise or consist of a substitution to R at the position corresponding to residue 315 of SEQ ID NO: 388; and a substitution to R at the position corresponding to residue 320 of SEQ ID NO: 388.

In certain embodiments, the one or more substitutions comprise or consist of three of the four positions corresponding to residue 315 of SEQ ID NO: 388, residue 318 of SEQ ID NO: 388, residue 319 of SEQ ID NO: 388, and residue 320 of SEQ ID NO: 388. For example, the three positions can be 315, 318, and 319; 315, 318, and 320; 318, 319, and 320; and 315, 319, and 320. In each instant or combination, the substitutions at each one of the two positions can be independently as described above for single substitutions at each position.

In certain embodiments, the one or more substitutions comprise or consist of all four positions corresponding to residue 315 of SEQ ID NO: 388, residue 318 of SEQ ID NO: 388, residue 319 of SEQ ID NO: 388, and residue 320 of SEQ ID NO: 388. In each instant or combination, the substitutions at each one of the two positions can be independently as described above for single substitutions at each position.

In certain embodiments, the integration activity at the pair of cognate integration recognition sites is at least about 200-fold, 300-fold, 400-fold, 500-fold, 600-fold, 700-fold, 800-fold, 900-fold, 1000-fold or more compared to the off-target integration activity at the pair of off-target integration recognition sites. For example, in certain embodiments, an engineered Bxb1 LSR may have an integration activity at the pair of cognate integration recognition sites (attB/attP) at least about 200-fold, 300-fold, 400-fold, 500-fold, 600-fold, 700-fold, 800-fold, 900-fold, 1000-fold or more compared to the off-target integration activity at the pair of off-target integration recognition sites (such as CAS031 or CAS421, See FIG. 59).

The integration activity of any eLSR can be assessed using any art-recognized method, and/or using any method as disclosed herein in, for example, the Examples (such as Examples 11-19).

### 6.9.2. Engineered Integration Enzyme with a Stabilization Domain

This disclosure further features integration enzymes (such as the eLSRs having a high fidelity mutation as described herein) are further engineered to include a domain (e.g., a stabilization domain) that increases stability of the integration enzyme when compared to an integration enzyme not engineered to include the domain (e.g., the stabilization domain).

In some embodiments, the integration enzyme or fragment thereof and the stabilization domain are fused, thereby by creating a fusion protein.

In one embodiment, an engineered integration enzyme comprises an integration enzyme or fragment thereof; an at least first stabilization domain; and a nuclear localization signal (NLS). In some embodiments, the integration enzyme or fragment thereof, the stabilization domain, and the NLS are fused, thereby by creating a fusion protein.

In some embodiments, the engineered integration enzyme is an integration enzyme described in Table 10 and engineered to include a stabilization domain that increases the stability of the BxB1 as compared to an Bxb1 integration enzyme not engineered to include the stabilization domain.

In some embodiments, the engineered integration enzyme is a BxB1 integration enzyme engineered to include a stabilization domain that increases the stability of the BxB1 as compared to an Bxb1 integration enzyme not engineered to include the stabilization domain.

In some embodiments, the engineered integration enzyme comprises an integration enzyme as described in International Patent Publication No. WO 2023/070031A2, which is hereby incorporated by reference in its entirety, where the integration enzyme is engineered to include a stabilization domain that increases the stability of the integration enzyme as compared to an integration enzyme not engineered to include the stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence selected from SEQ ID NOs: 378-393 and a stabilization domain, where the stabilization domain increases the stability of the engineered integration enzyme as compared to an integration enzyme not engineered to include the stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 378 and a stabilization domain that increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 379 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 380 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 381 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 382 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 383 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 384 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 385 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 386 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 387 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 388 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 389 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 390 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 391 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 392 and a stabilization domain, where the stabilization domain increase the stability of the integration enzyme as compared to an integration enzyme not engineered to include a stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 393 and a stabilization domain, where the stabilization domain increase the stability of the integration enzyme as compared to an integration enzyme not engineered to include a stabilization domain.

### 6.9.2.1 Stabilization Domains

In one embodiment, an engineered integration enzyme (such as the eLSR described herein) comprises an integration enzyme or fragment thereof; and an at least first stabilization domain. In some embodiments, the engineered integration enzyme comprises an integration enzyme or a fragment thereof, a first stabilization domain, and a second stabilization domain. In some embodiments, the engineered integration enzyme comprises an integration enzyme or a fragment thereof, a first stabilization domain, a second stabilization domain, and a third stabilization domain.

In some embodiments, the stabilization domain is selected from a stabilon motif, an exin2l motif, or a combination thereof.

In some embodiments, the stabilization domain comprises a stabilon domain. The stabilon sequence originated from C-terminus of p54 protein (drosophila). The stabilon amino acid sequence is conserved across species. The stabilon domain can be 13 amino acids (e.g., KDKKSDGKDSQKK (SEQ ID NO: 584) (Drosophila melanogaster)) or 15 amino acids (e.g., KDKDKKSDGKDSQKK (SEQ ID NO: 583)). In some embodiments, the stabilization domain comprises an amino acid sequence that is at least 80% (e.g., 80%, 85%, 90%, 91%, 92%, 93%, 94%, 95%, 96%, 97%, 99%, or 100%) identical to an amino acid sequence of KDKKSDGKDSQKK (SEQ ID NO: 584). In some embodiments, the stabilization domain comprises an amino acid sequence that is at least 80% (e.g., 80%, 85%, 90%, 91%, 92%, 93%, 94%, 95%, 96%, 97%, 99%, or 100%) identical to an amino acid sequence of KDKDKKSDGKDSQKK.

In some embodiments, the stabilization domain comprises a Exin21 domain. In some embodiments, the stabilization domain comprises an amino acid sequence of QPRFAAA (SEQ ID NO: 585). In some embodiments, the stabilization domain comprises an amino acid sequence having one, two, three, four or five or more amino acid substitutions in SEQ ID NO: 585. In some embodiments, the stabilization domain is encoded by a polynucleotide sequence having a nucleic acid sequence that is at least 80% (e.g., 80%, 85%, 90%, 91%, 92%, 93%, 94%, 95%, 96%, 97%, 99%, or 100%) identical to CAACCGCGGTTCGCGGCCGCT (SEQ ID NO: 586).

In some embodiments, the engineered integration enzymes comprises an integration enzyme comprising an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 388 and a stabilization domain that comprises an amino acid sequence that is at least 80% (e.g., 80%, 85%, 90%, 91%, 92%, 93%, 94%, 95%, 96%, 97%, 99%, or 100%) identical to an amino acid sequence of KDKDKKSDGKDSQKK (SEQ ID NO: 583) or an amino acid sequence having one, two, three, four or five or more amino acid substitutions in SEQ ID NO: 584.

In some embodiments, the engineered integration enzymes comprises an integration enzyme comprising an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 388 and a stabilization domain comprising an amino acid sequence of QPRFAAA (SEQ ID NO: 585) or an amino acid sequence having one, two, three, four or five or more amino acid substitutions in SEQ ID NO: 585.

In some embodiments, the stabilization domain is positioned relative to the integration enzyme (e.g., N-terminus, C-terminus, or in an internal position) such that when expressed/present in a cell, the stabilization domain increase stability of the integration enzyme (e.g., increase stability when compared to an integration enzyme not engineered to include the domain). In embodiments where the stabilization domain is placed in an internal position in the integration enzyme, the position in the integration enzyme is selected strategically placed such that it confers increased stability without compromising the integration enzyme's native function. For example, by positioning the stabilization domain internally, interference with the protein's natural structure and function can be avoided while still gaining the benefits of the stabilization domain.

In some embodiments, an engineered integration enzyme comprises an integration enzyme or a fragment thereof and a first stabilization domain, and optionally a second stabilization domain.

In some embodiments, the first stabilization domain and/or second stabilization domain are positioned in the engineered integration enzyme such that the engineered integration enzyme and the first and/or second stabilization domains are an in-frame fusion.

In some embodiments, the first stabilization domain and/or second stabilization domain are located N-terminus to the integration enzyme or fragment thereof, C-terminus to the integration enzyme of fragment thereof, or between two consecutive amino acid residues in the amino acid sequence of the integration enzyme or fragment thereof (also referred to as “internal”).

In some embodiments, the first stabilization domain and/or second stabilization domain are located N-terminus to the integration enzyme or fragment thereof.

In some embodiments, the first stabilization domain and/or second stabilization domain are located C-terminus to the integration enzyme or fragment thereof.

In some embodiments, the first stabilization domain and/or second stabilization domain are located between two consecutive amino acid residues in the amino acid sequence of the integration enzyme or fragment thereof.

In some embodiments, the two consecutive amino acid residues in the amino acid sequence of the integration enzyme are located in a N-terminal catalytic domain; a recombinase domain, or one or more zinc ribbon domain.

In some embodiments, the two consecutive amino acid residues in the amino acid sequence of the integration enzyme correspond amino acid residues between the N-terminal catalytic domain and the recombinase domain or between the recombinase domain and one of the zinc ribbon domains.

In some embodiments, the engineered integration enzyme comprises an orientation from N-terminus to C-terminus: N-I-S; I-S-N; S-I-N; N-S-I; N-X₁-I-S; I-X₁-S-N; S-X₁-I-N; N-X₁-S-I; N-I-X₁-S; I-S-X₁-N; S-I-X₁-N; N-S-X₁-I; N-X₁-I-X₂-S; I-X₁-S-X₂-N; S-X₁-I-X₂-N; and N-X₁-S-X₂-I; wherein I is the integration enzyme or fragment thereof, X₁ is a first linker, S is the stabilization domain, X₂ is a second linker, and N is the NLS.

### 6.9.3. Engineered Integration Enzymes with a Modification that Increase Stabilization

This disclosure features an integration enzyme (such as the eLSR described herein) where the amino acid sequence of the integration is modified such that the integration is more stable than an integration enzyme not comprising a modification in the amino acid sequence.

In one embodiment, an engineered integration enzyme comprises: an integration enzyme or fragment thereof comprising at least a first amino acid modification that increases the stability of the integration enzyme as compared to the integration enzyme not comprising the at least first amino acid modification. In some embodiments, the engineered integration enzyme comprises a nuclear localization signal (NLS), where the NLS is positioned in the engineered integration enzyme such that it directs the engineered integration enzyme to the nucleus.

In some embodiments, the engineered integration enzyme comprises an integration enzyme or fragment thereof comprising at least two (e.g., at least three, at least four, at least five, at least six, at least seven, at least eight, at least nine, or at least ten) amino acid modification such that the combination of the at least two modifications increase the stability of the integration enzyme as compared to the integration enzyme not comprising the at least first amino acid modification.

In some embodiments, the amino acid modification can be in a degron motif in the integration enzyme. In some embodiments, the amino acid modification can be a substitution, deletion, or insertion that increases the stability of the integration enzyme as compared to an integration enzyme not comprising the at least first amino acid modification.

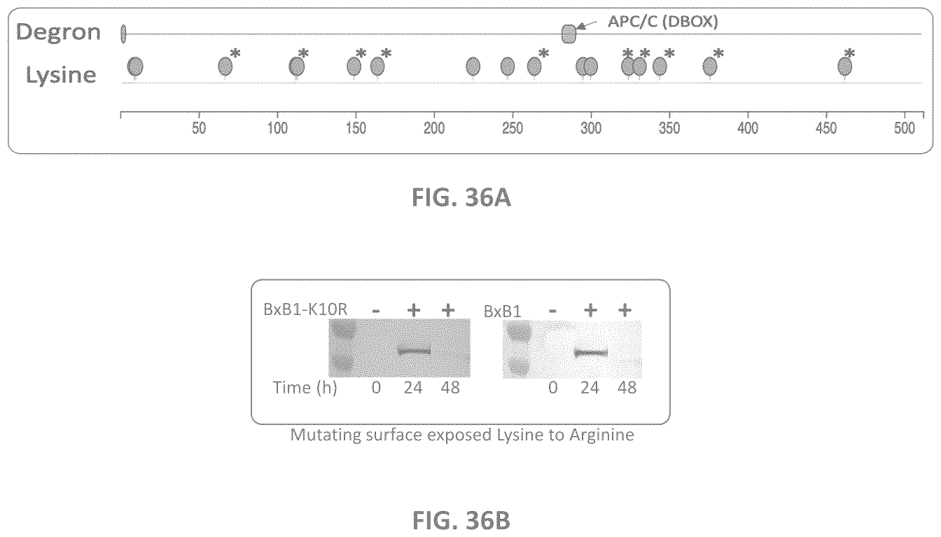

In some embodiments, the amino acid modification can be a substitution, deletion, or insertion in a degron motif in the integration enzyme. Computational prediction for BxB1 showed the existence of two degron domains and several lysine residues (FIG. 36A).

In some embodiments, the engineered integration enzyme is an integration enzyme described in Table 10 and comprises an amino acid modification that increases the stability of the integration enzyme as compared to an integration enzyme selected from Table 10 and not engineered to include the amino acid modification.

In some embodiments, the amino acid modification can be a substitution, deletion, or insertion in a BxB1 integration enzyme that increases the stability of the BxB1 as compared to an Bxb1 integration enzyme that does not comprise the at least first amino acid modification.

In some embodiments, the amino acid modification can be a substitution, deletion, or insertion in an integration enzyme having an amino acid sequence of SEQ ID NO: 388 that increases the stability of the integration enzyme as compared to an integration enzyme having a sequence of SEQ ID NO: 388 that does not comprise the at least first amino acid modification.

In one embodiment, the first amino acid modification is an amino acid substitution of L275V in SEQ ID NO: 388.

In some embodiments, the engineered integration enzyme comprises an integration enzyme as described in International Patent Publication No. WO 2023/070031A2, which is hereby incorporated by reference in its entirety, where the integration enzyme includes an amino acid modification that increases the stability of the integration enzyme as compared to an integration enzyme not engineered to have the amino acid modification.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence selected from SEQ ID NOs: 378-393 and an amino acid modification in the amino acid sequence where the modification increases the stability of the engineered integration enzyme as compared to an integration enzyme not engineered to have the at least first amino acid modification.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 378 and an amino acid modification in the sequence of SEQ ID NO: 378, where the amino acid modification increases the stability of the integration enzyme as compared to an integration enzyme having an amino acid sequence selected of SEQ ID NO: 378.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 379 and an amino acid modification in the sequence of SEQ ID NO: 379, where the amino acid modification increases the stability of the integration enzyme as compared to an integration enzyme having an amino acid sequence selected of SEQ ID NO: 379.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 380 and an amino acid modification in the sequence of SEQ ID NO: 380, where the amino acid modification increases the stability of the integration enzyme as compared to an integration enzyme having an amino acid sequence selected of SEQ ID NO: 381.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 381 and an amino acid modification in the sequence of SEQ ID NO: 381, where the amino acid modification increases the stability of the integration enzyme as compared to an integration enzyme having an amino acid sequence selected of SEQ ID NO: 381.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 382 and an amino acid modification in the sequence of SEQ ID NO: 382, where the amino acid modification increases the stability of the integration enzyme as compared to an integration enzyme having an amino acid sequence selected of SEQ ID NO: 382.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 383 and an amino acid modification in the sequence of SEQ ID NO: 383, where the amino acid modification increases the stability of the integration enzyme as compared to an integration enzyme having an amino acid sequence selected of SEQ ID NO: 383.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 384 and an amino acid modification in the sequence of SEQ ID NO: 384, where the amino acid modification increases the stability of the integration enzyme as compared to an integration enzyme having an amino acid sequence selected of SEQ ID NO: 384.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 385 and an amino acid modification in the sequence of SEQ ID NO: 385, where the amino acid modification increases the stability of the integration enzyme as compared to an integration enzyme having an amino acid sequence selected of SEQ ID NO: 385.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 386 and an amino acid modification in the sequence of SEQ ID NO: 386, where the amino acid modification increases the stability of the integration enzyme as compared to an integration enzyme having an amino acid sequence selected of SEQ ID NO: 386.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 387 and an amino acid modification in the sequence of SEQ ID NO: 387, where the amino acid modification increases the stability of the integration enzyme as compared to an integration enzyme having an amino acid sequence selected of SEQ ID NO: 387.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 388 and an amino acid modification in the sequence of SEQ ID NO: 388, where the amino acid modification increases the stability of the integration enzyme as compared to an integration enzyme having an amino acid sequence selected of SEQ ID NO: 388.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 389 and an amino acid modification in the sequence of SEQ ID NO: 389, where the amino acid modification increases the stability of the integration enzyme as compared to an integration enzyme having an amino acid sequence selected of SEQ ID NO: 389.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 390 and an amino acid modification in the sequence of SEQ ID NO: 390, where the amino acid modification increases the stability of the integration enzyme as compared to an integration enzyme having an amino acid sequence selected of SEQ ID NO: 390.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 391 and an amino acid modification in the sequence of SEQ ID NO: 391, where the amino acid modification increases the stability of the integration enzyme as compared to an integration enzyme having an amino acid sequence selected of SEQ ID NO: 391.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 392 and an amino acid modification in the sequence of SEQ ID NO: 392, where the amino acid modification increases the stability of the integration enzyme as compared to an integration enzyme having an amino acid sequence selected of SEQ ID NO: 392.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 393 and an amino acid modification in the sequence of SEQ ID NO: 393, where the amino acid modification increases the stability of the integration enzyme as compared to an integration enzyme having an amino acid sequence selected of SEQ ID NO: 393.

### 6.9.4. Engineered Integration Enzymes with a Combination of a Stabilization Domain and a Modification that Increases Stabilization

This disclosure also features engineered integration enzymes (such as the eLSR described herein) comprising a stabilization domain and an amino acid modification that increases the engineered integration enzymes stability.

In some embodiments, the engineered integration enzyme comprises an integration enzyme as described in International Patent Publication No. WO 2023/070031A2, which is hereby incorporated by reference in its entirety, where the integration enzyme includes a stabilization domain and an amino acid modification, where the stabilization domain, the amino acid modification, or both, increase the stability of the integration enzyme as compared to an integration enzyme not engineered to have the amino acid modification and/or the stabilization domain.

In some embodiments, the engineered integration enzyme is an integration enzyme described in Table 10 and comprises a stabilization domain and an amino acid modification in the same integration enzyme selected from Table 10, wherein the stabilization domain, the amino acid modification, or both, increase the stability of the integration enzyme as compared to an integration enzyme not engineered to have the amino acid modification and/or stabilization domain.

In some embodiments, the engineered integration enzyme is a BxB1 integration enzyme comprising a stabilization domain and an amino acid modification in the BxBT integration enzyme, wherein the stabilization domain, the amino acid modification, or both, increase the stability integration enzyme as compared to an integration enzyme not engineered to have the amino acid modification and/or stabilization domain.

In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence selected from SEQ ID NOs: 378-393, a stabilization domain, and an amino acid modification in the amino acid sequence, where the stabilization domain, the amino acid modification, or both, increase the stability of the engineered integration enzyme as compared to an integration enzyme not engineered to have the at amino acid modification and/or the stabilization domain. In some embodiments, the engineered integration enzyme comprises an amino acid sequence that is at least 80% (e.g., 80%, 85%, 90%, 91%, 92%, 93%, 94%, 95%, 96%, 97%, 99%, or 100%) identical to an amino acid sequence of SEQ ID NO: 388, a stabilization domain and an amino acid modification in SEQ ID NO: 388, wherein the stabilization domain, the amino acid modification, or both, increase the stability of integration enzyme as compared to an integration enzyme not engineered to have the amino acid modification and/or stabilization domain.

### 6.9.5. Engineered mRNA Encoding an Integration Enzyme

This disclosure features a polynucleotide comprising a nucleic acid sequence encoding any of the engineered integration enzymes described herein (see Section 4.9.1, 4.9.2, 4.9.3, and 4.9.4) or any of the engineered integration enzyme linked to the gene editor polypeptide or fusion proteins described herein (see Section 4.9.6).

In some embodiments, the nucleic acid sequence encoding the engineered integration enzyme is codon optimized. In some embodiments, the codon optimization is performed using an algorithm.

In some embodiments, the nucleic acid sequence of the engineered integration enzyme is optimized based on second structure such that the structure confers increased stability of the polynucleotide. In some embodiments, the optimization based on second structure relies on an algorithm to determine the optimal mRNA sequence.

A non-limiting example of an algorithm capable of being used to optimize the mRNA encoding any of the any of the engineered integration enzymes described herein (see Section 4.9.1, 4.9.2, and 4.9.3) is as described in Zhang et al. (Algorithm for Optimized mRNA Design Improves Stability and Immunogenicity, Nature 2023, doi.org/10.1038/s41586-023-06127-z), which is hereby incorporated by reference in its entirety. Zhang et al. refer to an algorithm called LinearDesign that optimizes both structural stability (via secondary structure) and codon usage.

In some embodiments, the nucleic acid sequence of the engineered integration enzyme is optimized based on the LinearDesign algorithm.

This disclosure also features vector comprising the nucleic acid sequence encoding any of the engineered integration enzymes described herein (see Section 4.9.1, 4.9.2, and 4.9.3) or any of the engineered integration enzyme linked to the gene editor polypeptide or fusion proteins described herein (see Section 4.9.5).

Other non-limiting examples of increasing the stability of an mRNA include: using novel UTR's or codons that increase the longevity of the mRNA or circular mRNA.

### 6.9.6. Engineered Integration Enzymes Fused to a Gene Editor Polypeptide

This disclosure also features engineered integration enzymes (e.g., any of the engineered integration enzymes described herein (see, e.g., Section 4.9.1, 4.9.2, 4.9.3, and 4.9.4) linked to a gene editor polypeptide (e.g., any of the gene editor polypeptides described herein). In such cases, the linked engineered integration enzyme-gene editor polypeptide can be used to incorporate an integration recognition site into the genome of a cell and for integrating a donor polynucleotide template into the genome.

In some embodiments, the C-terminus of the engineered integration enzyme is linked to the gene editor polypeptide.

In some embodiments, the C-terminus of the gene editor polypeptide is linked to the engineered integration enzymes.

In some embodiments, the engineered integration enzyme is linked to the gene editor polypeptide by in-frame fusion.

In some embodiments, the engineered integration enzyme is linked to the gene editor polypeptide by a linker.

In some embodiments, the linker is a peptide fused in-frame between the engineered integration enzyme and the gene editor polypeptide.

In some embodiments, the one or more linkers is selected from: Table 3.

In some embodiments, the gene editor polypeptide comprises a DNA binding domain and a reverse transcriptase.

This disclosure also features a fusion protein comprising (a) a DNA binding domain, optionally comprising a nickase activity; (b) a reverse transcriptase; and (c) any of the engineered integration enzymes described herein (see, e.g., Sections 4.9.1, 4.9.2, 4.9.3, and 4.9.4), wherein at least any two of elements (a), (b), or (c) are linked via at least a first C-terminal linker comprising. In some embodiments, the fusion protein comprises from N-terminus to C-terminus: (a), (b), and (c); (b), (a), and (c); (c), (a), and (b); (c), (b), and (a); (b), (c), and (a); and (b), (a), and (c).

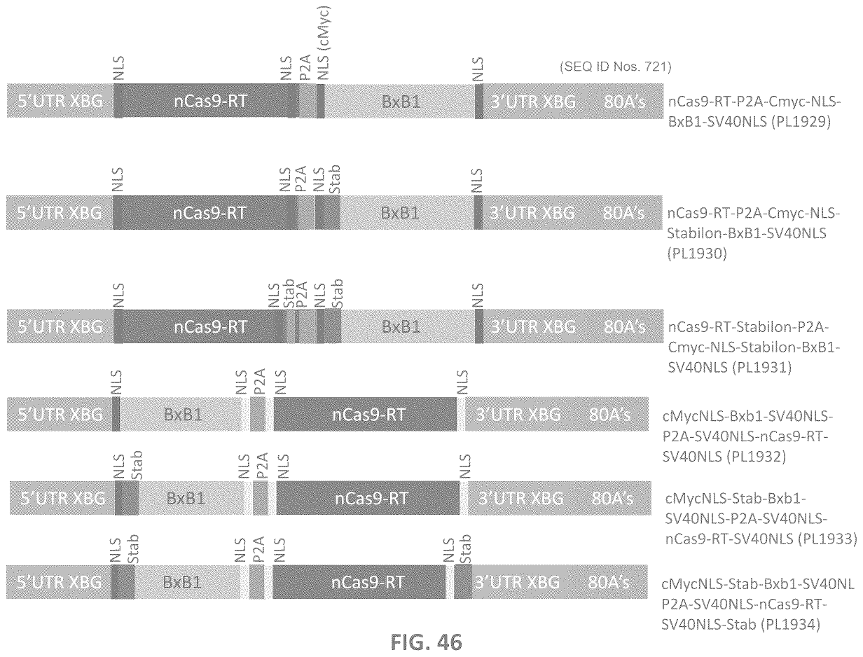

In some embodiments, the engineered integration enzyme is linked to a gene editor is described in FIG. 46.

### 6.9.7. Measuring Stability

Non-limiting examples of metrics that can be used as indication of protein stability include the following.

Protein Half-Life: This is the time required for half of the amount of protein in a cell to be degraded. This can be used to measure protein turnover. One non-limiting example of measuring protein half-life includes using pulse-chase experiments where the protein is labeled with a radioactive or other traceable isotope, then followed over time to see how rapidly it disappears.

Melting Temperature (Tm): This is a measure of a protein's thermal stability. It is the temperature at which half of the protein population is unfolded or denatured. It can be determined through a variety of methods, for example, without limitation, differential scanning calorimetry (DSC), circular dichroism (CD), or fluorescence-based thermal shift assays.

Thermodynamic Stability (AG): This is the change in Gibbs free energy between the folded and unfolded state of the protein. It measures how much energy is required to unfold the protein, with a larger value indicating a more stable protein. Non-limiting examples of measuring thermodynamic stability include equilibrium denaturation experiments, where the protein is exposed to varying concentrations of a denaturant and the degree of unfolding is monitored.

Protein Degradation Rate: This method measures how quickly a protein is degraded in a cell. It can be quantified using a variety of methods, for example, without limitation: western blot analysis (with or without the use of protease inhibitors) to assess protein levels over time.

Resistance to Chemical Denaturation: The concentration of denaturing agent (e.g., urea or guanidinium chloride) needed to unfold a protein can provide a measure of the proteins stability.

### 6.9.8. Systems Comprising an Engineered Integration Enzyme

This disclosure features a system for site-specifically integrating a donor polynucleotide template into a mammalian cell genome at a target DNA sequence, comprising: an attachment site containing gRNA (atgRNA) comprising at least a portion of an at least first integration recognition site; a gene editor polypeptide comprising a DNA binding nickase domain linked to a reverse transcriptase domain capable of incorporating the integration recognition site into the target DNA sequence, any of the engineered integration enzymes described herein (e.g., an engineered integration enzyme comprising an integration enzyme with a Hifi mutation (see Section 4.9.1) and optionally a stabilization domain (see Section 4.9.2) or an engineered integration enzyme comprising an integration enzyme comprising an amino acid modification that increases stability (see Section 4.9.3)); a donor polynucleotide template linked to a sequence that is an integration cognate of the integration recognition site present in the atgRNA, whereby the gene editor polypeptide site-specifically integrates the integration recognition site into the target DNA sequence, whereby the engineered integration enzyme integrates the donor polynucleotide template into the target DNA sequence.

In some embodiments of the system comprising an engineered integration enzyme (such as an eLSR described herein), the first atgRNA comprises: (i) a domain that is capable of guiding the gene editor polypeptide to the target DNA sequence; and (ii) a reverse transcriptase (RT) template that comprises at least a portion of an at least first integration recognition site, whereby the at least portion of the at least first integration recognition site is integrated into the genome of the cell at the target sequence.

In some embodiments of the system comprising an engineered integration enzyme, the system also includes a second atgRNA.

In some embodiments of the system comprising an engineered integration enzyme, the first atgRNA and the second atgRNA are an at least first pair of atgRNAs, wherein the at least first pair of atgRNAs have domains that are capable of guiding the gene editor polypeptide to the target DNA sequence; the first atgRNA further includes a first RT template that comprises at least a portion of an at least first integration recognition site; the second atgRNA further includes a second RT template that comprises at least a portion of the first integration recognition site, and the first atgRNA and the second atgRNAs collectively encode the entirety of the first integration recognition site, whereby the least first integration recognition site is integrated into the genome of the cell at the target sequence.

In some embodiments of the system comprising an engineered integration enzyme, upon introducing the system into the cell, the engineered integration enzyme enhances integration efficiency of the donor polynucleotide template at the site-specifically integrated integration recognition site as compared to the integration efficiency of a system using a non-engineered integration enzyme to integrate donor polynucleotide template at the site-specifically integrated integration recognition site.

### 6.9.9. Method of Using an Engineered Integration Enzyme

This disclosure also features a method for site-specifically integrating a donor polynucleotide template into a mammalian cell genome at a target DNA sequence, comprising: incorporating an integration recognition site into the genome by delivering into the cell: an attachment site containing guide RNA (atgRNA) comprising at least a portion of an at least first integration recognition site; and a gene editor polypeptide or polynucleotide encoding a gene editor polypeptide, wherein the gene editor polypeptide comprises a DNA binding nickase domain linked to a reverse transcriptase domain and is capable of incorporating the integration recognition site into the target DNA sequence; and optionally, a nicking gRNA; and integrating the donor polynucleotide template into the genome by delivering into the cell: any of the engineered integration enzymes described herein (e.g., an engineered integration enzyme comprising an integration enzyme and a Hifi mutation (see section 4.9.1) and optionally a stabilization domain (see Section 4.9.2) or an engineered integration enzyme comprising an integration enzyme comprising an amino acid modification that increases stability (see Section 4.9.3)); and a donor polynucleotide template, wherein the donor polynucleotide template is linked to a sequence that is an integration cognate of the integration recognition site present in the atgRNA, and wherein the donor polynucleotide template is integrated into the genome at the incorporated genomic integration recognition site by the integration enzyme.

In some embodiments of the method using the engineered integration enzyme, the first atgRNA comprises: (i) a domain that is capable of guiding the gene editor polypeptide to the target DNAsequence; and (ii) a reverse transcriptase (RT) template that comprises at least a portion of an at least first integration recognition site, whereby the at least portion of the at least first integration recognition site is integrated into the genome of the cell at the target sequence.

In some embodiments of the method using the engineered integration enzyme, the method also includes a second atgRNA.

In some embodiments of the method using the engineered integration enzyme, the first atgRNA and the second atgRNA are an at least first pair of atgRNAs, wherein the at least first pair of atgRNAs have domains that are capable of guiding the gene editor polypeptide to the target DNA sequence; the first atgRNA further includes a first RT template that comprises at least a portion of an at least first integration recognition site; the second atgRNA further includes a second RT template that comprises at least a portion of the first integration recognition site, and the first atgRNA and the second atgRNAs collectively encode the entirety of the first integration recognition site, whereby the least first integration recognition site is integrated into the genome of the cell at the target sequence.

In some embodiments of the method using the engineered integration enzyme, the method enhances integration efficiency of the donor polynucleotide template at the site-specifically integrated integration recognition stie at the double-stranded target DNA sequence as compared to the integration efficiency of the donor polynucleotide template at the site-specifically integrated recognition site when using a method that does not comprise the engineered integration enzyme.

### 6.10. Co-Delivery of Gene Editor and Donor DNA Template

This disclosure features methods of delivering (e.g., co-delivery or dual delivery) a system capable of site-specifically integrating a template polynucleotide into the genome of a cell, where the methods include delivering to a (i) gene editor construct and a (ii) template polynucleotide, and (iii) at least a first attachment site-containing guide (atgRNA).

This disclosure also features a method for delivering a system capable of site-specifically integrating a template polynucleotide into the genome of a cell, where the method includes: delivering a lipid nanoparticle (LNP) comprising a gene editor polynucleotide (e.g., a gene editor polynucleotide construct) and a vector comprising a template polynucleotide and at least a first attachment site-containing guide RNA (atgRNA). In some embodiments, the first atgRNA comprises (i) a domain that is capable of guiding the prime editor system to a target sequence; and (ii) a reverse transcriptase (RT) template that comprises at least a portion of a first integration recognition site. In some embodiments, where the vector comprises a polynucleotide encoding a first atgRNA, the RT template comprises the entirety of the first integration recognition site. In some embodiments, where the vector comprises a polynucleotide encoding a first atgRNA, the vector also includes a sequence encoding a nicking guide RNA (ngRNA).

This disclosure also features a method for delivering a system capable of site-specifically integrating a template polynucleotide into the genome of a cell, where the method includes: delivering a lipid nanoparticle (LNP) comprising a gene editor polynucleotide (e.g., a gene editor polynucleotide construct) and a vector comprising a template polynucleotide and a first attachment site-containing guide RNA (atgRNA) and a second attachment site-containing guide RNA (atgRNA). In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the at least first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap (e.g., 6 bp of complementarity).

This disclosure also features a method for delivering a system capable of site-specifically integrating a template polynucleotide into the genome of a cell, where the method includes: delivering into a cell a lipid nanoparticle (LNP) comprising: (i) a gene editor polynucleotide (e.g., a gene editor polynucleotide construct), and (ii) a first attachment site-containing guide RNA (atgRNA); and a vector comprising: (i) a template polynucleotide, and (ii) a second atgRNA. In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap (e.g., 6 bp of complementarity).

This disclosure also features a method for delivering a system capable of site-specifically integrating a template polynucleotide into the genome of a cell, where the method includes delivering: a lipid nanoparticle (LNP) comprising: (i) a gene editor polynucleotide (e.g., a gene editor polynucleotide construct), (ii) a first attachment site-containing guide RNA (atgRNA), and (iii) a second atgRNA; and a vector comprising (i) a template polynucleotide. In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the at least first integration recognition site; and the second atgRNA further includes a second RT template that comprises at least a portion of the at least first integration recognition site, and the first atgRNA and the second atgRNAs collectively encode the entirety of the first integration recognition site. In such embodiments, the first atgRNA and second atgRNA include at least a 6 bp overlap (e.g., 6 bp of complementarity).

This disclosure also features a method for delivering a system capable of site-specifically integrating a template polynucleotide into the genome of a cell, where the method includes delivering: a lipid nanoparticle (LNP) comprising: (i) a gene editor polynucleotide (e.g., a gene editor polynucleotide construct) and (ii) a first attachment site-containing guide RNA (atgRNA); and a vector comprising: (i) a template polynucleotide, and (ii) a nicking atgRNA. In some embodiments, the first atgRNA comprises (i) a domain that is capable of guiding the prime editor system to a target sequence; and (ii) a reverse transcriptase (RT) template that comprises at least a portion of a first integration recognition site. In some embodiments, where the vector comprises a polynucleotide encoding a first atgRNA, the RT template comprises the entirety of the first integration recognition site.

In some embodiments, where the method includes delivering an LNP and a first vector, the LNP and the first vector are delivered at least 1 day, at least 2 days, at least 3 days, at least 4 days, at least 5 days, at least 6 days, at least 7 days, at least 2 weeks, at least 3 weeks, at least 4 weeks, at least 5 weeks, at least 6 weeks, at least 7 weeks, or at least 8 weeks apart. In some embodiments, where the method includes delivering an LNP and a second vector, the LNP and the second vector are delivered a different times on the same day, at least 1 day, at least 2 days, at least 3 days, at least 4 days, at least 5 days, at least 6 days, at least 7 days, at least 2 weeks, at least 3 weeks, at least 4 weeks, at least 5 weeks, at least 6 weeks, at least 7 weeks, or 8 weeks apart. In some embodiments, the LNP and the first vector are delivered about 6 weeks apart.

This disclosure also features a method for delivering a system capable of site-specifically integrating a template polynucleotide into the genome of a cell, where the method includes delivering the system in vivo. In a non-limiting example, the system is delivered to a fetus or a neonate to site-specifically integrate in vivo a template polynucleotide into the genome of a cell. Delivering the system to a fetus or a neonate provides advantages over delivering the system later in life (e.g., after the neonate phase ends), including: (i) fewer number of cells that need to be treated (e.g., in the adult, there are trillions of cells, but in a fetus, there are significantly fewer cells); (ii) developmental benefits: the early stage of development of a fetus or a neonate means that if a genetic disease is treated successfully, the individual could potentially develop normally, with significant reduction or even complete removal of any of the disease manifestations; (iii) preventing disease progression: in certain genetic conditions the physiological damage is irreversible damage and in some instances is exacerbated as the disease progresses, therefore, intervening at the fetal (or neonate) stage, it is possible to prevent or reduce the progression of the disease and potentially prevent any irreversible damage from occurring; (iv) higher cell turnover and cell division rate: in a developing fetus, cells are dividing rapidly as the fetus (or neonate) grows, which means that if programmable gene insertion is achieved in the fetus (or neonate) is introduced, it could be propagated more rapidly throughout the body than in an older child or adult; and (v) immune tolerance: for example, there is evidence to suggest that performing gene therapy (e.g., programmable gene insertion) early in development might result in immune tolerance to the vector, thereby reducing the risk of an immune response against the system.

In some embodiments, the method includes delivering an LNP and a first vector, the LNP and the first vector are delivered to a cell in vivo. In some embodiments, the in vivo cells are present in a fetus or a neonate. In some embodiments, the LNP is delivered between age 0 (day of birth) and age 7 days and the vector is delivered between age 5 weeks and age 7 weeks. In some embodiments, the LNP is delivered at about at 2 days and the vector is delivered at about age 6 weeks.

In some embodiments, where the method includes delivering an LNP and a first vector, the LNP and the first vector are delivered to a cell in vivo, the LNP can be delivered to a fetus at a first time point and the vector is delivered to the fetus after the fetus is born (referred to after birth as a neonate). In some embodiments, the LNP is delivered to a fetus and the vector is delivered to the fetus after birth (i.e., at the neonate stage) at any point between birth and up to age 8 weeks. In some embodiments, the LNP is delivered to the fetus and the vector is delivered at about age 1 week, 2 weeks, 3 weeks, 4 weeks, 5 weeks, 6 weeks, 7 weeks or 8 weeks.

In some embodiments, where the method includes delivering an LNP and a first vector, the LNP and the first vector are delivered to a cell in vivo, the LNP can be delivered to a fetus at a first time point and the vector is delivered to the fetus (child) after the fetus (child) is born, for example, when the child is age 90 days or older (e.g., age 6 months, age 9 months, age 1 year, age 2 years, age 3 years, age 4 years, age 5 years, age 6 years, or older).

As used herein, the term “fetus” refers to an unborn offspring. As used herein, the term “neonate” refers to a newborn infant, which includes the first 90 days of life. When referring to mice, neonate can refer to animals up to 10 days of age.

This disclosure also features a system capable of site-specifically integrating a template polynucleotide into the genome of a cell, the system comprising: a lipid nanoparticle (LNP) comprising a gene editor polynucleotide (e.g., a gene editor polynucleotide construct) and a vector comprising a template polynucleotide and at least a first attachment site-containing guide RNA (atgRNA). In some embodiments, the first atgRNA comprises (i) a domain that is capable of guiding the prime editor system to a target sequence; and (ii) a reverse transcriptase (RT) template that comprises at least a portion of a first integration recognition site. In some embodiments, where the vector comprises a polynucleotide encoding a first atgRNA, the RT template comprises the entirety of the first integration recognition site. In some embodiments, where the vector comprises a polynucleotide encoding a first atgRNA, the vector also includes a sequence encoding a nicking guide RNA (ngRNA).

This disclosure also features a system capable of site-specifically integrating a template polynucleotide into the genome of a cell, the system comprising: a lipid nanoparticle (LNP) comprising a gene editor polynucleotide (e.g., a gene editor polynucleotide construct) and a vector comprising a template polynucleotide and a first attachment site-containing guide RNA (atgRNA) and a second attachment site-containing guide RNA (atgRNA). In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap.

This disclosure also features a system capable of site-specifically integrating a template polynucleotide into the genome of a cell, the system comprising: a lipid nanoparticle (LNP) comprising: (i) a gene editor polynucleotide (e.g., a gene editor polynucleotide construct), and (ii) a first attachment site-containing guide RNA (atgRNA); and a vector comprising: (i) a template polynucleotide, and (ii) a second atgRNA. In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap.

This disclosure also features a system capable of site-specifically integrating a template polynucleotide into the genome of a cell, the system comprising: co-delivering: a lipid nanoparticle (LNP) comprising: (i) a gene editor polynucleotide (e.g., a gene editor polynucleotide construct), (ii) a first attachment site-containing guide RNA (atgRNA), and (iii) a second atgRNA; and a vector comprising (i) a template polynucleotide. In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; and the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap.

This disclosure also features a system capable of site-specifically integrating a template polynucleotide into the genome of a cell, the system comprising: a lipid nanoparticle (LNP) comprising: (i) a gene editor polynucleotide (e.g., a gene editor polynucleotide construct) and (ii) a first attachment site-containing guide RNA (atgRNA); and a vector comprising: (i) a template polynucleotide, and (ii) a nicking atgRNA. In some embodiments, the first atgRNA comprises (i) a domain that is capable of guiding the prime editor system to a target sequence; and (ii) a reverse transcriptase (RT) template that comprises at least a portion of a first integration recognition site. In some embodiments, where the vector comprises a polynucleotide encoding a first atgRNA, the RT template comprises the entirety of the first integration recognition site.

In typical embodiments, the LNP comprising a gene editor polynucleotide construct is capable delivering to a cell cytoplasm the gene editor polynucleotide construct. In some embodiments, the LNP comprising a gene editor polynucleotide construct is capable delivering to a cell nucleus the gene editor polynucleotide construct. In some embodiments, the LNP comprises a gene editor protein and associated guide nucleic acids. In some embodiments, the LNP comprises a gene editor protein and associated guide nucleic acids that are capable of localizing to cell nucleus.

In some embodiments, a gene editor polynucleotide construct is delivered to a cell by a fusosome. In some embodiments, a gene editor polynucleotide construct is delivered to a cell cytoplasm by a fusosome. In some embodiments, the fusosome comprises a gene editor protein and associated guide nucleic acids.

In some embodiments, a gene editor polynucleotide construct is delivered to a cell by an exosome. In some embodiments, a gene editor polynucleotide construct is delivered to a cell cytoplasm by an exosome. In some embodiments, the exosome comprises a gene editor protein and associated guide nucleic acids.

In some embodiments, the prime editor or Gene Writer protein fusion, either of which may have a fused/linked integrase, is incorporated (i.e., packaged) into LNP as protein. Further, associated atgRNA and optional ngRNAs may be co-packaged with gene editor proteins in LNP.

In some embodiments, the gene editor polynucleotide construct comprises (a) a polynucleotide sequence encoding a prime editor fusion protein or a Gene Writer™ protein, (b) a polynucleotide sequence encoding an attachment site-containing guide RNA (atgRNA), (c) optionally, a polynucleotide sequence encoding a nickase guide RNA (ngRNA), (d) a polynucleotide sequence encoding an integrase, (e) and optionally, a polynucleotide sequence encoding a recombinase.

In some embodiments, the prime editor or Gene Writer protein fusion, either of which may have a fused/linked integrase, is expressed as a split construct. In typical embodiments, the split construct in reconstituted in a cell. In some embodiments, the split construct can be fused or ligated via intein protein splicing. In some embodiments, the split construct can be reconstituted via protein-protein inter-molecular bonding and/or interactions. In some embodiments, the split construct can be reconstituted via chemical, biological, or environmental induced oligomerization. In certain embodiments, the split construct can be adapted into one or more nucleic acid constructs described herein.

### 6.10.1. Gene Editor Polynucleotide

In some embodiments, the systems described include a gene editor polynucleotide that is delivered to a cell using the methods described herein. In some embodiments, the gene editor polynucleotide is delivered as a polynucleotide (e.g., an mRNA). In some embodiments, the gene editor polynucleotide is delivered as a protein. In some embodiments, the gene editor polynucleotide or protein is packaged, and thereby vectorized, within a lipid nanoparticle (LNP). In some embodiments, the gene editor polynucleotide or protein is packaged in a LNP and is co-delivered with a template polynucleotide (i.e., nucleic acid “cargo” or nucleic acid “payload”) packaged into a separate vector (e.g., a viral vector (e.g., an AAV or adenovirus)) or a second lipid nanoparticle (LNP).

In some embodiments, the gene editor polynucleotide is delivered to the cells as a polynucleotide. For example, the gene editor polynucleotide is delivered to the cells as an mRNA encoding the gene editor polynucleotide (e.g., the gene editor protein or the prime editor system). In some embodiments, the mRNA comprises one or more modified uridines. In some embodiments, the mRNA comprises a sequence where each of the uridines is a modified uridine. In some embodiments, the mRNA is uridine depleted. In some embodiments, the mRNA encoding the nickase comprises one or more modified uridines. In some embodiments, the mRNA encoding the reverse transcriptase comprises one or more modified uridines. In some embodiments, the mRNA encoding the nickase comprises one or more modified uridines, and the mRNA encoding the reverse transcriptase comprises one or more modified uridines. In some embodiments, where the integrase is encoded in an mRNA, the mRNA comprises modified uridines. In some embodiments, a modified uridine is a N1-Methylpseudouridine-5′-Triphosphate. In some embodiments, a modified uridine is a pseudouridine. In some embodiments, the mRNA comprises a 5′ cap. In some embodiments, the 5′ cap comprises a molecular formula of C32H43N15024P4 (free acid).

In some embodiments, the gene editor polynucleotide (e.g., a gene editor polynucleotide construct) comprises a polynucleotide sequence encoding a primer editor system (e.g., any of the prime editor systems described herein). In some embodiments, the prime editor system comprises a nucleotide sequence encoding a nickase (e.g., any of the Cas proteins or variants thereof (e.g., nickases) and nickases described herein, see Tables 4-8) and a nucleotide sequence encoding a reverse transcriptase (e.g., any of the reverse transcriptases described herein). In some embodiments, the nucleotide sequence encoding the nickase and the nucleotide sequence encoding the reverse transcriptase are positioned in the construct such that when expressed the nickase is linked to the reverse transcriptase. In some embodiments, the nickase is linked to the reverse transcriptase by in-frame fusion. In some embodiments, the nickase is linked to the reverse transcriptase by a linker. In some embodiments, the linker is a peptide fused in-frame between the nickase and reverse transcriptase.

In some embodiments, the gene editor polynucleotide (e.g., a gene editor polynucleotide construct) further comprises a polynucleotide sequence encoding at least a first integrase (e.g., any of the integrases described herein, e.g., as described in Table 10 and also in Yamall et al., Nat. Biotechnol., 2022, doi.org/10.1038/s41587-022-01527-4 and Durrant et al., Nat. Biotechnol., 2022, doi.org/10.1038/s41587-022-01494-w, each of which are herein incorporated by reference in their entireties), wherein the first integrase comprises a Hifi mutation described herein (see section 4.9.1) and optionally a stabilon (see section 4.9.2). In some embodiments, the linked nickase-reverse transcriptase are further linked to the first integrase.

In some embodiments, the gene editor polynucleotide construct further comprises a polynucleotide sequence encoding at least a first recombinase (e.g., any of the recombinases described herein).

### 6.10.2. Split Nickase and Reverse Transcriptase

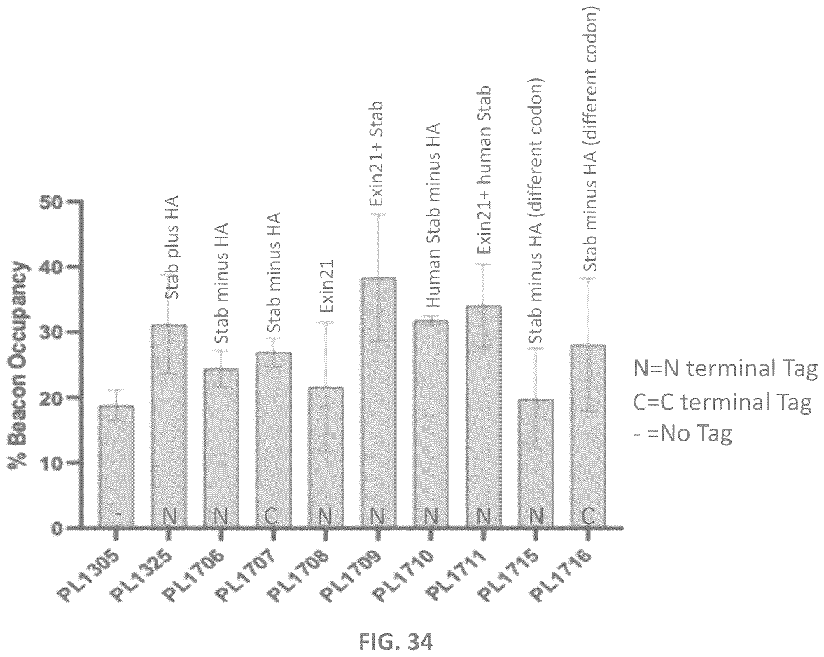

Also provided herein are prime editor systems where the nucleotide sequence encoding the nickase and the nucleotide sequence encoding the reverse transcriptase are positioned in a construct such that when expressed the nickase is not linked to the reverse transcriptase. In a non-limiting example, a sequence encoding a self-cleaving peptide (e.g., a P2A) is positioned between the nucleotide sequence encoding the nickase and the nucleotide sequence encoding the reverse transcriptase such that when expressed the nickase is not linked to the reverse transcriptase (see, e.g., FIG. 34).

Also provided herein are prime editor systems where the nucleotide sequence encoding the nickase and the nucleotide sequence encoding the reverse transcriptase are on separate polynucleotides. In some embodiments where the prime editor system includes separate polynucleotides encoding the nickase and the reverse transcriptase, the nickase and/or the reverse transcriptase can be engineered such that when expressed they are linked to a binding domain, where the binding domain is part of a binding pair that enables recruitment of the reverse transcriptase to the genomic location of the nickase (e.g., nCas9). A binding pair can be any pair of molecules wherein upon forming the binding pair the binding domains enable recruitment of the reverse transcriptase to the genomic location of the nickase. Once in sufficient proximity with each other the binding domains can dimerize. This dimerization (or other type of interaction) brings the nickase and RT into proximity with each other, thereby enhancing the reverse transcription of the RT template in the atgRNA.

Non-limiting examples of binding pairs that can aid recruitment of the RT to the genomic location of the nickase include without limitation: dimerizing leucine zippers, an antigen and a corresponding antigen binding domains (e.g., antibody), RNA aptamers and corresponding RNA aptamer binding proteins, affinity tags and corresponding affinity tag binding proteins.

In some embodiments, dimerizing leucine zippers that can serve as a binding pair includes two binding domains: a Leucine Zip LZ1 domain having a sequence with at least 80%, 85%, 90%, 95%, or 99% sequence identity to a sequence of SEQ ID NO: 568 and a Leucine Zip LZ2 domain having a sequence with at least 80%, 85%, 90%, 95%, or 99% sequence identity to a sequence of SEQ ID NO: 569. In one embodiment, the Leucine Zip LZ1 domain is positioned either N-terminally or C-terminally to the nickase and the Leucine Zip LZ2 is positioned either N-terminally or C-terminally to the RT. In one embodiment, the Leucine Zip LZ1 domain is positioned either N-terminally or C-terminally to the RT and the Leucine Zip LZ2 domain is positioned either N-terminally or C-terminally to the Cas9.

| SEQ ID | | |
|---|---|---|
| NO: | ame | Sequence |
| 724 | eucine | RLEIRAAFLRQRNTALRTEVAE |
| | Zip LZ1 | LEQEVQRLENEVSQYETRYGPL |
| | domain | GGGK |
| 725 | eucine | LEIEAAFLERENTALETRVAEL |
| | Zip LZ2 | RQRVQRLRNRVSQYRTRYGPLG |
| | domain | GGKSGGSSGGSSGSETPGTSES |
| | | ATPESSGGSSGGSS |

In one embodiment, the system and methods described herein include a construct that encodes a polypeptide having a sequence with at least 80%, 85%, 90%, 95%, or 99% sequence identity to a sequence of SEQ ID NO: 576 (a Leucine Zip LZ1 domain positioned C-terminally to the nickase), and a construct that encodes a polypeptide having a sequence with at least 80%, 85%, 90%, 95%, or 99% sequence identity to a sequence of SEQ ID NO: 577 (a Leucine Zip LZ2 domain positioned N-terminally to the RT).

In one embodiment, the system and methods described herein include a construct that encodes a polypeptide having a sequence with at least 80%, 85%, 90%, 95%, or 99% sequence identity to a sequence of SEQ ID NO: 578 (a Leucine Zip LZ1 domain positioned N-terminally to the nickase), and a construct that encodes a polypeptide having a sequence with at least 80%, 85%, 90%, 95%, or 99% sequence identity to a sequence of SEQ ID NO: 577 (a Leucine Zip LZ2 domain positioned N-terminally to the RT).

In some embodiments, dimerizing leucine zippers that can serve as a binding pair includes two binding domains: a EE₁₂₃₄L peptide having a sequence with at least 80%, 85%, 90%, 95%, or 99% sequence identity to a sequence of SEQ ID NO: 570 and a RR₁₂₃₄L peptide having a sequence with at least 80%, 85%, 90%, 95%, or 99% sequence identity to a sequence of SEQ ID NO: 571. In one embodiment, the EE₁₂₃₄L peptide is positioned either N-terminally or C-terminally to the nickase and the RR₁₂₃₄L peptide is positioned either N-terminally or C-terminally to the RT. In one embodiment, the EE₁₂₃₄L peptide is positioned either N-terminally or C-terminally to the RT and the RR₁₂₃₄L peptide is positioned either N-terminally or C-terminally to the Cas9.

| EQ ID | | |
|---|---|---|
| NO: | ame | Sequence |
| 726 | E₁₂₃₄L | LEIEAAFLEQENTALETEVAEL |
| | peptide | LEQEVQRLENIVSQYETRYGPG |
| | | GGK |
| 727 | R₁₂₃₄L | KGGGLEIRAAFLRRRNTALRTR |
| | peptide | VAELRQRVQRLRNIVSQYETRY |
| | | GPL |

In one embodiment, the system and methods described herein include a construct that encodes a polypeptide having a sequence with at least 80%, 85%, 90%, 95%, or 99% sequence identity to a sequence of SEQ ID NO: 579 (a EE₁₂₃₄L peptide positioned N-terminally to the nickase), and a construct that encodes a polypeptide having a sequence with at least 80%, 85%, 90%, 95%, or 99% sequence identity to a sequence of SEQ ID NO: 580 (a RR1234L peptide positioned N-terminally to the RT).

In some embodiments, an affinity tag is a SpyTag and the corresponding affinity tag binding protein is SpyCatcher, where upon binding of the SpyTag with the SpyCatcher the fusion protein is a covalently stabilized multi-protein complex. In one embodiment, the affinity tag (e.g., SpyTag) is positioned either N-terminally or C-terminally to the nickase and the affinity tag binding protein (e.g, SpyCatcher) is positioned either N-terminally or C-terminally to the RT. In one embodiment, the affinity tag (e.g., SpyTag) is positioned either N-terminally or C-terminally to the RT and the affinity tag binding protein (e.g, SpyCatcher) is positioned either N-terminally or C-terminally to the nickase.

In some embodiments, an antigen and corresponding antigen binding domain includes an antigen such as a peptide present within a tag or a tag itself and an antigen binding domain that binds specifically to the antigen. In one embodiments, an antigen is a GCN4 peptide within a SunTag and the antigen binding domain is an anti-GCN4 scFv. In one embodiment, the SunTag (GCN4 peptide) is positioned either N-terminally or C-terminally to the nickase and the anti-GCN4 antigen binding domain is positioned either N-terminally or C-terminally to the RT. In one embodiment, the SunTag (GCN4 peptide) is positioned either N-terminally or C-terminally to the RT and the anti-GCN4 antigen binding domain is positioned either N-terminally or C-terminally to the nickase.

In one embodiment, the system and methods described herein include a construct that encodes a polypeptide having a sequence with at least 80%, 85%, 90%, 95%, or 99% sequence identity to a sequence of SEQ ID NO: 574 (SunTag (GCN4 peptide) positioned C-terminally to the nickase), and a construct that encodes a polypeptide having a sequence with at least 80%, 85%, 90%, 95%, or 99% sequence identity to a sequence of SEQ ID NO: 575 (anti-GCN4 antigen binding domain positioned N-terminally to the RT).

In some embodiments, provided herein are prime editor systems where a construct includes a nucleotide sequence encoding a self-cleaving peptide (e.g., a P2A) positioned between the nucleotide sequence encoding the nickase and the nucleotide sequence encoding the RT. In such cases, the position of the nucleotide sequence encoding the nickase, the nucleotide sequence encoding the self-cleaving peptide (e.g., a P2A), and the nucleotide sequence encoding the RT is such that when expressed the nickase is not linked to the reverse transcriptase. In one embodiment, the construct includes the nucleotide sequences positioned 5′-3′: nucleotide sequence encoding the nickase, nucleotide sequence encoding the self-cleaving peptide (e.g., a P2A), and nucleotide sequence encoding the RT. In one embodiment, the construct includes the nucleotide sequences positioned 5′-3′: nucleotide sequence encoding the RT, nucleotide sequence encoding the self-cleaving peptide (e.g., a P2A), and nucleotide sequence encoding the nickase.

In one embodiment, the construct that includes a nucleotide sequence encoding a self-cleaving peptide (e.g., a P2A) positioned between the nucleotide sequence encoding the nickase and the nucleotide sequence encoding the RT has an amino acid sequence with at least 80%, 85%, 90%, 95%, or 99% sequence identity to a sequence of SEQ ID NO: 581 or 582 (see FIG. 34).

| SEQ ID | | |
|---|---|---|
| NO: | Name | Sequence |
| 747 | PL1427 | MKRTADGSEFESPKKKRKVDKKYSIGLDIGTNSVGWAVITDEYKVPSKKFKVLGN |
| | | TDRHSIKKNLIGALLFDSGETAEATRLKRTARRRYTRRKNRICYLQEIFSNEMAK |
| | | VDDSFFHRLEESFLVEEDKKHERHPIFGNIVDEVAYHEKYPTIYHLRKKLVDSTD |
| | | KADLRLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFKSNFD |
| | | LAEDAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYIDGGA |
| | | SQEEFYKFIKPILEKMDGTEELLVKLNREDLLRKQRTFDNGSIPHQIHLGELHAI |
| | | LRRQEDFYPFLKDNREKIEKILTFRIPYYVGPLARGNSRFAWMTRKSEETITPWN |
| | | FEEVVDKGASAQSFIERMTNFDKNLPNEKVLPKHSLLYEYFTVYNELTKVKYVTE |
| | | GMRKPAFLSGEQKKAIVDLLFKTNRKVTVKQLKEDYFKKIECFDSVEISGVEDRF |
| | | NASLGTYHDLLKIIKDKDFLDNEENEDILEDIVLTLTLFEDREMIEERLKTYAHL |
| | | FDDKVMKQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDELVKVMG |
| | | RHKPENIVIEMARENQTTQKGQKNSRERMKRIEEGIKELGSQILKEHPVENTQLQ |
| | | NEKLYLYYLQNGRDMYVDQELDINRLSDYDVDAIVPQSFLKDDSIDNKVLTRSDK |
| | | NRGKSDNVPSEEVVKKMKNYWRQLLNAKLITQRKFDNLTKAERGGLSELDKAGFI |
| | | KRQLVETRQITKHVAQILDSRMNTKYDENDKLIREVKVITLKSKLVSDFRKDFQF |
| | | YKVREINNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKKYGGFDSP |
| | | TVAYSVLWVAKVEKGKSKKLKSVKELLGITIMERSSFEKNPIDFLEAKGYKEVKK |
| | | DLIIKLPKYSLFELENGRKRMLASAGELQKGNELALPSKYVNFLYLASHYEKLKG |
| | | SPEDNEQKQLFVEQHKHYLDEIIEQISEFSKRVILADANLDKVLSAYNKHRDKPI |
| | | REQAENIIHLFTLTNLGAPAAFKYFDTTIDRKRYTSTKEVLDATLIHQSITGLYE |
| | | TRIDLSQLGGDGSGSGEELLSKNYHLENEVARLKKGSGSGEELLSKNYHLENEVA |
| | | RLKKSGGSKRTADGSEFESPKKKRKVGGSVSGWRLFKKIS** |
| 748 | PL1428 | MKRTADGSEFESPKKKRKVGPDIVMTQSPSSLSASVGDRVTITCRSSTGAVTTSN |
| | | YASWVQEKPGKLFKGLIGGTNNRAPGVPSRFSGSLIGDKATLTISSLQPEDFATY |
| | | FCALWYSNHWVFGQGTKVELKRGGGGSGGGGSGGGGSSGGGSEVKLLESGGGLVQ |
| | | PGGSLKLSCAVSGFSLTDYGVNWVRQAPGRGLEWIGVIWGDGITDYNSALKDRFI |
| | | ISKDNGKNTVYLQMSKVRSDDTALYYCVTGLFDYWGQGTLVTVSSGSGLNIEDEY |
| | | RLHETSKEPDVSLGSTWLSDFPQAWAETGGMGLAVRQAPLIIPLKATSTPVSIKQ |
| | | YPMSQEARLGIKPHIQRLLDQGILVPCQSPWNTPLLPVKKPGTNDYRPVQDLREV |
| | | NKRVEDIHPTVPNPYNLLSGLPPSHQWYTVLDLKDAFFCLRLHPTSQPLFAFEWR |
| | | DPEMGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQRWLTEA |
| | | RKETVMGQPTPKTPRQLREFLGKAGFCRLFIPGFAEMAAPLYPLTKPGTLFNWGP |
| | | DQQKAYQEIKQALLTAPALGLPDLTKPFELFVDEKQGYAKGVLTQKLGPWRRPVA |
| | | YLSKKLDPVAAGWPPCLRMVAAIAVLTKDAGKLTMGQPLVILAPHAVEALVKQPP |
| | | DRWLSNARMTHYQALLLDTDRVQFGPVVALNPATLLPLPEEGLQHNCLDILAEAH |
| | | GTRPDLTDQPLPDADHTWYTDGSSLLQEGQRKAGAAVTTETEVIWAKALPAGTSA |
| | | QRAELIALTQALKMAEGKKLNVYTDSRYAFATAHIHGEIYRRRGWLTSEGKEIKN |
| | | KDEILALLKALFLPKRLSIIHCPGHQKGHSAEARGNRMADQAARKAAITETPDTS |
| | | TLLIENSSPSGGSKRTADGSEFEPKKKRKVGGSVSGWRLFKKIS** |
| 749 | PL1597 | MKRTADGSEFESPKKKRKVDKKYSIGLDIGTNSVGWAVITDEYKVPSKKFKVLGN |
| | | TDRHSIKKNLIGALLFDSGETAEATRLKRTARRRYTRRKNRICYLQEIFSNEMAK |
| | | VDDSFFHRLEESFLVEEDKKHERHPIFGNIVDEVAYHEKYPTIYHLRKKLVDSTD |
| | | KADLRLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFKSNFD |
| | | LAEDAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYIDGGA |
| | | SQEEFYKFIKPILEKMDGTEELLVKLNREDLLRKQRTFDNGSIPHQIHLGELHAI |
| | | LRRQEDFYPFLKDNREKIEKILTFRIPYYVGPLARGNSRFAWMTRKSEETITPWN |
| | | FEEVVDKGASAQSFIERMTNFDKNLPNEKVLPKHSLLYEYFTVYNELTKVKYVTE |
| | | GMRKPAFLSGEQKKAIVDLLFKTNRKVTVKQLKEDYFKKIECFDSVEISGVEDRF |
| | | NASLGTYHDLLKIIKDKDFLDNEENEDILEDIVLTLTLFEDREMIEERLKTYAHL |
| | | FDDKVMKQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDELVKVMG |
| | | RHKPENIVIEMARENQTTQKGQKNSRERMKRIEEGIKELGSQILKEHPVENTQLQ |
| | | NEKLYLYYLQNGRDMYVDQELDINRLSDYDVDAIVPQSFLKDDSIDNKVLTRSDK |
| | | NRGKSDNVPSEEVVKKMKNYWRQLLNAKLITQRKFDNLTKAERGGLSELDKAGFI |
| | | KRQLVETRQITKHVAQILDSRMNTKYDENDKLIREVKVITLKSKLVSDFRKDFQF |
| | | YKVREINNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKKYGGFDSP |
| | | TVAYSVLWVAKVEKGKSKKLKSVKELLGITIMERSSFEKNPIDFLEAKGYKEVKK |
| | | DLIIKLPKYSLFELENGRKRMLASAGELQKGNELALPSKYVNFLYLASHYEKLKG |
| | | SPEDNEQKQLFVEQHKHYLDEIIEQISEFSKRVILADANLDKVLSAYNKHRDKPI |
| | | REQAENIIHLFTLTNLGAPAAFKYFDTTIDRKRYTSTKEVLDATLIHQSITGLYE |
| | | TRIDLSQLGGDGGGSPKKKRKVGGGGSGGRLEIRAAFLRQRNTALRTEVAELEQE |
| | | VQRLENEVSQYETRYGPLGGGK** |
| 750 | PL1598 | MLEIEAAFLERENTALETRVAELRQRVQRLRNRVSQYRTRYGPLGGGKSGGSSGG |
| | | SSGSETPGTSESATPESSGGSSGGSSKRTADGSEFESPKKKRKVLNIEDEYRLHE |
| | | TSKEPDVSLGSTWLSDFPQAWAETGGMGLAVRQAPLIIPLKATSTPVSIKQYPMS |
| | | QEARLGIKPHIQRLLDQGILVPCQSPWNTPLLPVKKPGTNDYRPVQDLREVNKRV |
| | | EDIHPTVPNPYNLLSGLPPSHQWYTVLDLKDAFFCLRLHPTSQPLFAFEWRDPEM |
| | | GISGQLTWTRLPQGFKNSPTLFNEALHRDLADFRIQHPDLILLQYVDDLLLAATS |
| | | ELDCQQGTRALLQTLGNLGYRASAKKAQICQKQVKYLGYLLKEGQRWLTEARKET |
| | | VMGQPTPKTPRQLREFLGKAGFCRLFIPGFAEMAAPLYPLTKPGTLFNWGPDQQK |
| | | AYQEIKQALLTAPALGLPDLTKPFELFVDEKQGYAKGVLTQKLGPWRRPVAYLSK |
| | | KLDPVAAGWPPCLRMVAAIAVLTKDAGKLTMGQPLVILAPHAVEALVKQPPDRWL |
| | | SNARMTHYQALLLDTDRVQFGPVVALNPATLLPLPEEGLQHNCLDILAEAHGTRP |
| | | DLTDQPLPDADHTWYTDGSSLLQEGQRKAGAAVTTETEVIWAKALPAGTSAQRAE |
| | | LIALTQALKMAEGKKLNVYTDSRYAFATAHIHGEIYRRRGWLTSEGKEIKNKDEI |
| | | LALLKALFLPKRLSIIHCPGHQKGHSAEARGNRMADQAARKAAITETPDTSTLLI |
| | | ENSSPSGGSKRTADGSEFEPKKKRKVGGSVSGWRLFKKIS** |
| 578 | PL1599 | MRLEIRAAFLRQRNTALRTEVAELEQEVQRLENEVSQYETRYGPLGGGKGGGGSG |
| | | GKRTADGSEFESPKKKRKVDKKYSIGLDIGTNSVGWAVITDEYKVPSKKFKVLGN |
| | | TDRHSIKKNLIGALLFDSGETAEATRLKRTARRRYTRRKNRICYLQEIFSNEMAK |
| | | VDDSFFHRLEESFLVEEDKKHERHPIFGNIVDEVAYHEKYPTIYHLRKKLVDSTD |
| | | KADLRLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFKSNFD |
| | | LAEDAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYIDGGA |
| | | SQEEFYKFIKPILEKMDGTEELLVKLNREDLLRKQRTFDNGSIPHQIHLGELHAI |
| | | LRRQEDFYPFLKDNREKIEKILTFRIPYYVGPLARGNSRFAWMTRKSEETITPWN |
| | | FEEVVDKGASAQSFIERMTNFDKNLPNEKVLPKHSLLYEYFTVYNELTKVKYVTE |
| | | GMRKPAFLSGEQKKAIVDLLFKTNRKVTVKQLKEDYFKKIECFDSVEISGVEDRF |
| | | NASLGTYHDLLKIIKDKDFLDNEENEDILEDIVLTLTLFEDREMIEERLKTYAHL |
| | | FDDKVMKQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDELVKVMG |
| | | RHKPENIVIEMARENQTTQKGQKNSRERMKRIEEGIKELGSQILKEHPVENTQLQ |
| | | NEKLYLYYLQNGRDMYVDQELDINRLSDYDVDAIVPQSFLKDDSIDNKVLTRSDK |
| | | NRGKSDNVPSEEVVKKMKNYWRQLLNAKLITQRKFDNLTKAERGGLSELDKAGFI |
| | | KRQLVETRQITKHVAQILDSRMNTKYDENDKLIREVKVITLKSKLVSDFRKDFQF |
| | | YKVREINNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKKYGGFDSP |
| | | TVAYSVLWVAKVEKGKSKKLKSVKELLGITIMERSSFEKNPIDFLEAKGYKEVKK |
| | | DLIIKLPKYSLFELENGRKRMLASAGELQKGNELALPSKYVNFLYLASHYEKLKG |
| | | SPEDNEQKQLFVEQHKHYLDEIIEQISEFSKRVILADANLDKVLSAYNKHRDKPI |
| | | REQAENIIHLFTLTNLGAPAAFKYFDTTIDRKRYTSTKEVLDATLIHQSITGLYE |
| | | TRIDLSQLGGDGGGSPKKKRKV** |
| 579 | PL1600 | MKRTADGSEFESPKKKRKVDKKYSIGLDIGTNSVGWAVITDEYKVPSKKFKVLGN |
| | | TDRHSIKKNLIGALLFDSGETAEATRLKRTARRRYTRRKNRICYLQEIFSNEMAK |
| | | VDDSFFHRLEESFLVEEDKKHERHPIFGNIVDEVAYHEKYPTIYHLRKKLVDSTD |
| | | KADLRLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFKSNFD |
| | | LAEDAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYIDGGA |
| | | SQEEFYKFIKPILEKMDGTEELLVKLNREDLLRKQRTFDNGSIPHQIHLGELHAI |
| | | LRRQEDFYPFLKDNREKIEKILTFRIPYYVGPLARGNSRFAWMTRKSEETITPWN |
| | | FEEVVDKGASAQSFIERMTNFDKNLPNEKVLPKHSLLYEYFTVYNELTKVKYVTE |
| | | GMRKPAFLSGEQKKAIVDLLFKTNRKVTVKQLKEDYFKKIECFDSVEISGVEDRF |
| | | NASLGTYHDLLKIIKDKDFLDNEENEDILEDIVLTLTLFEDREMIEERLKTYAHL |
| | | FDDKVMKQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDELVKVMG |
| | | RHKPENIVIEMARENQTTQKGQKNSRERMKRIEEGIKELGSQILKEHPVENTQLQ |
| | | NEKLYLYYLQNGRDMYVDQELDINRLSDYDVDAIVPQSFLKDDSIDNKVLTRSDK |
| | | NRGKSDNVPSEEVVKKMKNYWRQLLNAKLITQRKFDNLTKAERGGLSELDKAGFI |
| | | KRQLVETRQITKHVAQILDSRMNTKYDENDKLIREVKVITLKSKLVSDFRKDFQF |
| | | YKVREINNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKKYGGFDSP |
| | | TVAYSVLWVAKVEKGKSKKLKSVKELLGITIMERSSFEKNPIDFLEAKGYKEVKK |
| | | DLIIKLPKYSLFELENGRKRMLASAGELQKGNELALPSKYVNFLYLASHYEKLKG |
| | | SPEDNEQKQLFVEQHKHYLDEIIEQISEFSKRVILADANLDKVLSAYNKHRDKPI |
| | | REQAENIIHLFTLTNLGAPAAFKYFDTTIDRKRYTSTKEVLDATLIHQSITGLYE |
| | | TRIDLSQLGGDGGGSPKKKRKVGGGGSGGLEIEAAFLEQENTALETEVAELEQEV |
| | | QRLENIVSQYETRYGPLGGGK** |
| 580 | PL1601 | MKGGGLEIRAAFLRRRNTALRTRVAELRQRVQRLRNIVSQYETRYGPLGGGGSGG |
| | | KRTADGSEFESPKKKRKVLNIEDEYRLHETSKEPDVSLGSTWLSDFPQAWAETGG |
| | | MGLAVRQAPLIIPLKATSTPVSIKQYPMSQEARLGIKPHIQRLLDQGILVPCQSP |
| | | WNTPLLPVKKPGTNDYRPVQDLREVNKRVEDIHPTVPNPYNLLSGLPPSHQWYTV |
| | | LDLKDAFFCLRLHPTSQPLFAFEWRDPEMGISGQLTWTRLPQGFKNSPTLFNEAL |
| | | HRDLADFRIQHPDLILLQYVDDLLLAATSELDCQQGTRALLQTLGNLGYRASAKK |
| | | AQICQKQVKYLGYLLKEGQRWLTEARKETVMGQPTPKTPRQLREFLGKAGFCRLF |
| | | IPGFAEMAAPLYPLTKPGTLFNWGPDQQKAYQEIKQALLTAPALGLPDLTKPFEL |
| | | FVDEKQGYAKGVLTQKLGPWRRPVAYLSKKLDPVAAGWPPCLRMVAAIAVLTKDA |
| | | GKLTMGQPLVILAPHAVEALVKQPPDRWLSNARMTHYQALLLDTDRVQFGPVVAL |
| | | NPATLLPLPEEGLQHNCLDILAEAHGTRPDLTDQPLPDADHTWYTDGSSLLQEGQ |
| | | RKAGAAVTTETEVIWAKALPAGTSAQRAELIALTQALKMAEGKKLNVYTDSRYAF |
| | | ATAHIHGEIYRRRGWLTSEGKEIKNKDEILALLKALFLPKRLSIIHCPGHQKGHS |
| | | AEARGNRMADQAARKAAITETPDTSTLLIENSSPSGGSKRTADGSEFEPKKKRKV |
| | | GGSVSGWRLFKKIS** |
| 581 | PL1697 | MKRTADGSEFESPKKKRKVDKKYSIGLDIGTNSVGWAVITDEYKVPSKKFKVLGN |
| | | TDRHSIKKNLIGALLFDSGETAEATRLKRTARRRYTRRKNRICYLQEIFSNEMAK |
| | | VDDSFFHRLEESFLVEEDKKHERHPIFGNIVDEVAYHEKYPTIYHLRKKLVDSTD |
| | | KADLRLIYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNFKSNFD |
| | | LAEDAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYIDGGA |
| | | SQEEFYKFIKPILEKMDGTEELLVKLNREDLLRKQRTFDNGSIPHQIHLGELHAI |
| | | LRRQEDFYPFLKDNREKIEKILTFRIPYYVGPLARGNSRFAWMTRKSEETITPWN |
| | | FEEVVDKGASAQSFIERMTNFDKNLPNEKVLPKHSLLYEYFTVYNELTKVKYVTE |
| | | GMRKPAFLSGEQKKAIVDLLFKTNRKVTVKQLKEDYFKKIECFDSVEISGVEDRF |
| | | NASLGTYHDLLKIIKDKDFLDNEENEDILEDIVLTLTLFEDREMIEERLKTYAHL |
| | | FDDKVMKQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDELVKVMG |
| | | RHKPENIVIEMARENQTTQKGQKNSRERMKRIEEGIKELGSQILKEHPVENTQLQ |
| | | NEKLYLYYLQNGRDMYVDQELDINRLSDYDVDAIVPQSFLKDDSIDNKVLTRSDK |
| | | NRGKSDNVPSEEVVKKMKNYWRQLLNAKLITQRKFDNLTKAERGGLSELDKAGFI |
| | | KRQLVETRQITKHVAQILDSRMNTKYDENDKLIREVKVITLKSKLVSDFRKDFQF |
| | | YKVREINNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKKYGGFDSP |
| | | TVAYSVLWVAKVEKGKSKKLKSVKELLGITIMERSSFEKNPIDFLEAKGYKEVKK |
| | | DLIIKLPKYSLFELENGRKRMLASAGELQKGNELALPSKYVNFLYLASHYEKLKG |
| | | SPEDNEQKQLFVEQHKHYLDEIIEQISEFSKRVILADANLDKVLSAYNKHRDKPI |
| | | REQAENIIHLFTLTNLGAPAAFKYFDTTIDRKRYTSTKEVLDATLIHQSITGLYE |
| | | TRIDLSQLGGDGGGSPKKKRKVGSGATNFSLLKQAGDVEENPGPKRTADGSEFES |
| | | PKKKRKVLNIEDEYRLHETSKEPDVSLGSTWLSDFPQAWAETGGMGLAVRQAPLI |
| | | IPLKATSTPVSIKQYPMSQEARLGIKPHIQRLLDQGILVPCQSPWNTPLLPVKKP |
| | | GTNDYRPVQDLREVNKRVEDIHPTVPNPYNLLSGLPPSHQWYTVLDLKDAFFCLR |
| | | LHPTSQPLFAFEWRDPEMGISGQLTWTRLPQGFKNSPTLFNEALHRDLADFRIQH |
| | | PDLILLQYVDDLLLAATSELDCQQGTRALLQTLGNLGYRASAKKAQICQKQVKYL |
| | | GYLLKEGQRWLTEARKETVMGQPTPKTPRQLREFLGKAGFCRLFIPGFAEMAAPL |
| | | YPLTKPGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTMGQPLVI |
| | | LAPHAVEALVKQPPDRWLSNARMTHYQALLLDTDRVQFGPVVALNPATLLPLPEE |
| | | GLQHNCLDILAEAHGTRPDLTDQPLPDADHTWYTDGSSLLQEGQRKAGAAVTTET |
| | | EVIWAKALPAGTSAQRAELIALTQALKMAEGKKLNVYTDSRYAFATAHIHGEIYR |
| | | RRGWLTSEGKEIKNKDEILALLKALFLPKRLSIIHCPGHQKGHSAEARGNRMADQ |
| | | AARKAAITETPDTSTLLIENSSPSGGSKRTADGSEFEPKKKRKV** |
| 582 | PL1698 | MKRTADGSEFESPKKKRKVLNIEDEYRLHETSKEPDVSLGSTWLSDFPQAWAETG |
| | | GMGLAVRQAPLIIPLKATSTPVSIKQYPMSQEARLGIKPHIQRLLDQGILVPCQS |
| | | PWNTPLLPVKKPGTNDYRPVQDLREVNKRVEDIHPTVPNPYNLLSGLPPSHQWYT |
| | | VLDLKDAFFCLRLHPTSQPLFAFEWRDPEMGISGQLTWTRLPQGFKNSPTLFNEA |
| | | LHRDLADFRIQHPDLILLQYVDDLLLAATSELDCQQGTRALLQTLGNLGYRASAK |
| | | KAQICQKQVKYLGYLLKEGQRWLTEARKETVMGQPTPKTPRQLREFLGKAGFCRL |
| | | FIPGFAEMAAPLYPLTKPGTLFNWGPDQQKAYQEIKQALLTAPALGLPDLTKPFE |
| | | LFVDEKQGYAKGVLTQKLGPWRRPVAYLSKKLDPVAAGWPPCLRMVAAIAVLTKD |
| | | AGKLTMGQPLVILAPHAVEALVKQPPDRWLSNARMTHYQALLLDTDRVQFGPWVA |
| | | LNPATLLPLPEEGLQHNCLDILAEAHGTRPDLTDQPLPDADHTWYTDGSSLLQEG |
| | | QRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGH |
| | | SAEARGNRMADQAARKAAITETPDTSTLLIENSSPSGGSKRTADGSEFEPKKKRK |
| | | VGSGATNFSLLKQAGDVEENPGPKRTADGSEFESPKKKRKVDKKYSIGLDIGTNS |
| | | VGWAVITDEYKVPSKKFKVLGNTDRHSIKKNLIGALLFDSGETAEATRLKRTARR |
| | | RYTRRKNRICYLQEIFSNEMAKVDDSFFHRLEESFLVEEDKKHERHPIFGNIVDE |
| | | VAYHEKYPTIYHLRKKLVDSTDKADLRLIYLALAHMIKFRGHFLIEGDLNPDNSD |
| | | VDKLFIQLVQTYNQLFEENPINASGVDAKAILSARLSKSRRLENLIAQLPGEKKN |
| | | GLFGNLIALSLGLTPNFKSNFDLAEDAKLQLSKDTYDDDLDNLLAQIGDQYADLF |
| | | LAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLR |
| | | KQRTFDNGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKNLPNEKVLPK |
| | | HSLLYEYFTVYNELTKVKYVTEGMRKPAFLSGEQKKAIVDLLFKTNRKVTVKQLK |
| | | EDYFKKIECFDSVEISGVEDRFNASLGTYHDLLKIIKDKDFLDNEENEDILEDIV |
| | | LTLTLFEDREMIEERLKTYAHLFDDKVMKQLKRRRYTGWGRLSRKLINGIRDKQS |
| | | GKTILDFLKSDGFANRNFMQLIHDDSLTFKEDIQKAQVSGQGDSLHEHIANLAGS |
| | | PAIKKGILQTVKVVDELVKVMGRHKPENIVIEMARENQTTQKGQKNSRERMKRIE |
| | | EGIKELGSQILKEHPVENTQLQNEKLYLYYLQNGRDMYVDQELDINRLSDYDVDA |
| | | IVPQSFLKDDSIDNKVLTRSDKNRGKSDNVPSEEVVKKMKNYWRQLLNAKLITQR |
| | | KFDNLTKAERGGLSELDKAGFIKRQLVETRQITKHVAQILDSRMNTKYDENDKLI |
| | | REVKVITLKSKLVSDFRKDFQFYKVREINNYHHAHDAYLNAVVGTALIKKYPKLE |
| | | SEFVYGDYKVYDVRKMIAKSEQEIGKATAKYFFYSNIMNFFKTEITLANGEIRKR |
| | | PLIETNGETGEIVWDKGRDFATVRKVLSMPQVNIVKKTEVQTGGFSKESILPKRN |
| | | SDKLIARKKDWDPKKYGGFDSPTVAYSVLWVAKVEKGKSKKLKSVKELLGITIME |
| | | RSSFEKNPIDFLEAKGYKEVKKDLIIKLPKYSLFELENGRKRMLASAGELQKGNE |
| | | LALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKR |
| | | YTSTKEVLDATLIHQSITGLYETRIDLSQLGGDGGGSPKKKRKV** |

### 6.10.3. Supplementing with Reverse Transcriptase

This disclosure also features systems and methods that include supplementing with reverse transcriptase. In some embodiments, the system and methods include supplementation with a polynucleotide encoding a supplemental reverse transcriptase (i.e., a separate polynucleotide from the gene editor polynucleotide, which includes a polynucleotide encoding a reverse transcriptase). Systems and methods that include supplementation with reverse transcriptase increase integration efficiency of the at least first integration recognition site into the genome of the cell when compared to systems and methods that do not include supplementation with reverse transcriptase. These systems and methods refer to the supplemental or supplemental reverse transcriptase molecule as “supplementing” or “supplementation” with reverse transcriptase.

In some embodiments, the supplemental reverse transcriptase is the same as the reverse transcriptase that is included in the gene editor polynucleotide. In some embodiments, the supplemental reverse transcriptase is different from the reverse transcriptase that is included in the gene editor polynucleotide.

In some embodiments, the polynucleotide encoding the supplemental reverse transcriptase is an mRNA.

In some embodiments, the supplemental reverse transcriptase is delivered concurrently with the one or more LNP, one or more atgRNA, and the template polynucleotide. In some embodiments, the supplemental reverse transcriptase is delivered to the cell prior to delivering the one or more LNP, one or more atgRNA, and the template polynucleotide to the cell. In some embodiments, the supplemental reverse transcriptase is delivered to the cell after delivering the one or more LNP, one or more atgRNA, and the template polynucleotide to the cell.

Also provided herein are methods of increasing integration efficiency of the at least first integration recognition site into the genome of the cell, where the method comprises delivering to the cell one or more LNPs, one or more vectors, one or more template polynucleotides and supplementing with a polynucleotide encoding a supplemental reverse transcriptase, where the increase in integration efficiency is in comparison to methods that do not include supplementation with a polynucleotide encoding RT. In such embodiments, the method includes a ratio of 1:1, 1:2, 1:3, 1:4, 1:5, 1:6, 1:7, 1:8, 1:9, or 1:10 of gene editor polynucleotide to polynucleotide encoding the supplemental reverse transcriptase.

Also provided herein are methods of increasing integration efficiency of the at least first integration recognition site into the genome of the cell, where the method comprises delivering to the cell one or more LNPs, one or more vectors, one or more template polynucleotides, where the gene editor polynucleotide comprises a ratio of a ratio of 1:1, 1:2, 1:3, 1:4, 1:5, 1:6, 1:7, 1:8, 1:9, 1:10 of the nucleotide sequence encoding the nickase to the nucleotide sequence encoding the reverse transcriptase, and where the increase in integration efficiency is in comparison to methods that do not include supplementation with a polynucleotide encoding RT.

### 6.10.3.1 Supplementation of RT when Using a Fused Design

In some embodiments, the system and methods described herein include supplementation with a polynucleotide encoding a reverse transcriptase, where the polynucleotide is in addition to the gene editor polynucleotide that also includes a polynucleotide encoding a reverse transcriptase.

In one embodiment, a method for delivering a system capable of site-specifically integrating a template polynucleotide into the genome of a cell, includes delivering one or more LNPs and one or more vectors, and also includes delivering a separate polynucleotide encoding a supplemental reverse transcriptase (RT). In a non-limiting example, a method for delivering a system capable of site-specifically integrating a template polynucleotide into the genome of a cell comprises delivering the gene editor polynucleotide (e.g., a polynucleotide encoding a nickase and a reverse transcriptase either linked or not linked), one or more atgRNA, a template polynucleotide, and a polynucleotide encoding a supplemental reverse transcriptase.

In some embodiments, the method includes delivering into the cell a ratio of gene editor polynucleotide to polynucleotide encoding the supplemental RT of 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:32, 1:34, 1:36, 1:38, or 1:40 or more. In some embodiments, the method includes delivering into the cell a gene editor polynucleotide and a polynucleotide encoding the supplemental RT, where the polynucleotide encoding the supplemental RT is delivered at 1.5 times (1.5×), 2×, 3×, 4×, 5×, 6×, 7×, 8×, 9×, 10×, 20×, 30×, 40×, 50×, 60×, 70×, 80×, 90×, or 100× or more the amount of the gene editor polynucleotide.

In some embodiments, the method includes delivering into the cell a ratio of polynucleotide encoding the nickase to polynucleotide encoding the RT of 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:32, 1:34, 1:36, 1:38, or 1:40 or more. In some embodiments, the method includes delivering into the cell a polynucleotide encoding the nickase to polynucleotide encoding the RT, where the polynucleotide encoding the RT is delivered at 1.5 times (1.5×), 2×, 3×, 4×, 5×, 6×, 7×, 8×, 9×, 10×, 20×, 30×, 40×, 50×, 60×, 70×, 80×, 90×, or 100× or more the amount of the nickase polynucleotide.

In some embodiments, the systems and methods that include delivering of the gene editor polynucleotide supplemented with a polynucleotide encoding a supplemental RT increases integration efficiency of the at least first integration recognition site into the genome of the cell when compared to systems and methods that include delivering to the cell a gene editor polynucleotide not supplemented with a polynucleotide encoding supplemental reverse transcriptase.

### 6.10.3.2 Split Design Plus Further Supplementation

In some embodiments, the system and methods described herein include supplementation with a polynucleotide encoding a reverse transcriptase, where the system includes a gene editor polynucleotide split over two polynucleotides: a first polynucleotide encoding the nickase and a second polynucleotide encoding the reverse transcriptase. In such cases, the first and second polynucleotides can be delivered to the cell at a ratio of 1:1 and supplementation is achieved by adding a polynucleotide encoding a supplemental reverse transcriptase. In such cases, the supplementation comprises adding the polynucleotide encoding the supplemental reverse transcriptase at a ratio of 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:32, 1:34, 1:36, 1:38, or 1:40 (gene editor polynucleotide (e.g., the first and second polynucleotides delivered to the cell at a ratio of 1:1) to the polynucleotide encoding the supplemental reverse transcriptase).

In some embodiments, a method of delivering to the cell the one or more atgRNA and the gene editor polynucleotide (where the nickase and the RT are encoded on different polynucleotides and delivered at a ratio 1:1 (nickase to RT)) and supplementing with a polynucleotide encoding a supplemental RT increases integration efficiency of the at least first integration recognition site into the genome of the cell when compared to methods that do not include supplementing with a polynucleotide encoding a supplemental RT.

### 6.10.3.3 Supplementation Via Increase in Ratios when Using Split Design

In some embodiments, the system and methods described herein include supplementation with a polynucleotide encoding a reverse transcriptase, where the system includes a gene editor polynucleotide split over two polynucleotides: a first polynucleotide encoding the nickase and a second polynucleotide encoding the reverse transcriptase, and supplementation is achieved by increasing the amount of the second polynucleotide encoding the reverse transcriptase.

In some embodiments, where the gene editor polynucleotide is such that the nucleotide sequence encoding the nickase and the nucleotide sequence encoding the reverse transcriptase are not linked, supplementation of a supplemental reverse transcriptase refers to delivering a greater than 1:1 ratio of nickase to reverse transcriptase (e.g., a 1:2 or greater ratio). In a non-limiting example, the gene editor polynucleotide delivered to the cell in a ratio 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:32, 1:34, 1:36, 1:38, or 1:40 of polynucleotide sequence encoding the nickase to the polynucleotide sequence encoding the reverse transcriptase.

In some embodiments, a method of delivering to the cell the one or more atgRNA and the gene editor polynucleotide where the nickase and the RT are encoded on different polynucleotides and delivered at a ratio greater than 1:1 (nickase to RT) increases integration efficiency of the at least first integration recognition site into the genome of the cell when compared to methods comprising delivering to the cell a gene editor polynucleotide at a ratio of 1:1 (nickase to RT).

### 6.10.3.4 Supplementation of RT Via Vector Design

In some embodiments, the system and methods described herein include supplementing with reverse transcriptase, where in supplementing comprises using controllable expression (e.g., inducible promoters, strong promoters) of a polynucleotide encoding a reverse transcriptase.

In one embodiment, the polynucleotide encoding the nickase and the polynucleotide encoding the RT are on a dual promoter vector, where each polynucleotide is controlled by a different promoter. In a non-limiting example, the polynucleotide encoding the RT is operably linked to a stronger promoter than the polynucleotide encoding the nickase, whereby the RT is expressed at higher levels than the nickase. In another non-limiting example, the polynucleotide encoding the RT is operably linked to an inducible promoter, whereby inducing expression of the RT results in higher levels of the RT compared to the nickase. In another non-limiting example, the polynucleotide encoding the nCas9 is operably linked to an inducible promoter, whereby inducing expressing of the nCas9 results in lower levels of the nCas9 than the RT.

### 6.10.4. Vector

In some embodiments, the systems and methods described herein include a vector that is capable of co-delivering a template polynucleotide, one or more attachment site-containing gRNA, one or more integrases, one or more recombinases, a gene editor polynucleotide, one or more integration recognition sites, one or more recombinase recognition sites, or a combination thereof.

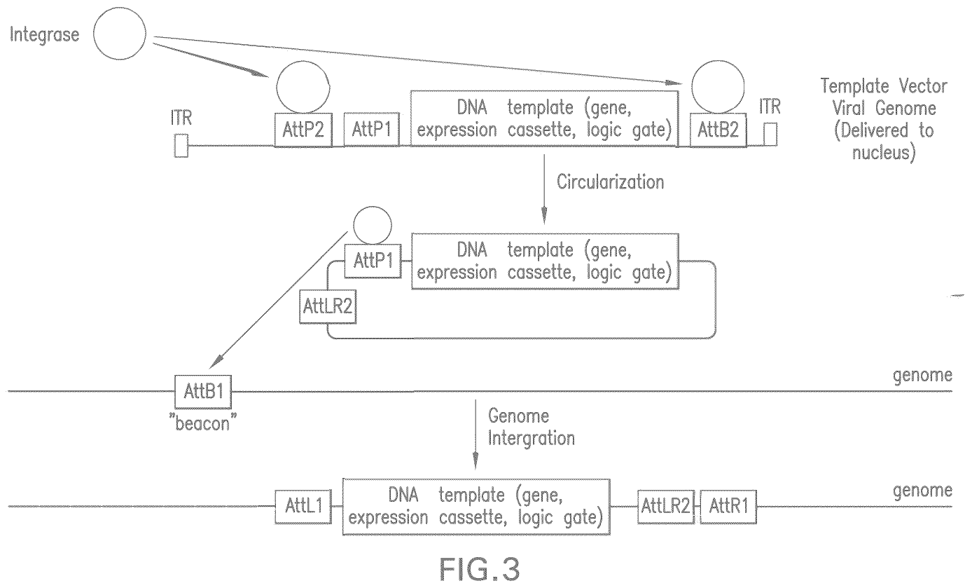

Non-limiting examples of vectors that can be used in the methods or systems described herein include the vectors described in FIGS. 3-6.

### 6.10.4.1 AtgRNA and/or ngRNA

In some embodiments, the vector includes a polynucleotide sequence encoding an attachment site-containing guide RNA (atgRNA). In such embodiments, the polynucleotide sequence encoding the attachment site-containing guide RNA (atgRNA) is operably linked to a regulatory element (e.g., a U6 promoter) that is capable of driving expression of the atgRNA. In such embodiments, the atgRNA comprises (i) a domain that is capable of guiding the prime editor system to a target sequence; and (ii) a reverse transcriptase (RT) template that comprises at least a portion of a first integration recognition site. In some embodiments, where the system, and thereby the vector, include a polynucleotide encoding only a first atgRNA, the RT template comprises the entirety of the first integration recognition site. In such embodiments, the vector or the LNP includes a polynucleotide sequence encoding a nicking gRNA.

In some embodiments, the vector includes a polynucleotide sequence encoding a first attachment site-containing guide RNA (atgRNA) and a polynucleotide sequence encoding a second attachment site-containing guide RNA (atgRNA). In such embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the a first integration recognition site; the second atgRNA further includes a second RT template that comprises at least a portion of the first integration recognition site, and the first atgRNA and the second atgRNAs collectively encode the entirety of the first integration recognition site. In such embodiments, the first atgRNA and second atgRNA include at least a 6 bp overlap.

### 6.10.4.2 Template Polynucleotide

In typical embodiments, the vector includes a template polynucleotide and a sequence that is an integration cognate of an integration recognition site site-specifically incorporated into the genome of a cell. For example, the vector includes a template polynucleotide and a second integration recognition site that is a cognate pair with the first integration recognition site site-specifically incorporated into the genome of the cell. In such embodiments, the sequence that is an integration cognate (e.g., a second integration recognition site) enables integration of the template polynucleotide or portion thereof when contacted with an integrase and the site-specifically incorporated first integration recognition site.

In typical embodiments, the vector comprising a template polynucleotide is a recombinant adenovirus, a helper dependent adenovirus, an AAV, a lentivirus, an HSV, an annelovirus, a retrovirus, a Doggybone™ DNA (dbDNA), a minicircle, a plasmid, a miniDNA, an exosome, a fusosome, or an nanoplasmid. In preferred embodiments, the vector is capable of localizing to the nucleus.

In certain embodiments, the template polynucleotide is delivered to the cytoplasm and localizes to the nucleus. In certain embodiments, the template polynucleotide is delivered to the cytoplasm by LNP. In certain embodiments, the donor template polynucleotide construct comprises a recognition sequence that is recognized by a DNA binding protein (DNA binding domain) or a transcription factor binding domain. In certain embodiments, the donor template polynucleotide construct is delivered to the nucleus by an integrase or recombinase.

In certain embodiments, the template polynucleotide is delivered to the mitochondria. In certain embodiments, the donor template polynucleotide construct comprises a mitochondria targeting sequence.

In certain embodiments, the vector comprising a template polynucleotide is AAV. In some embodiments, the AAV contains a 5′ inverted terminal repeat (ITR). In some embodiments, the AAV contains a 3′ inverted terminal repeat (ITR). In some embodiments, the AAV contains a 5′ and a 3′ ITR. In some embodiments, the 5′ and 3′ ITR are not derived from the same serotype of virus. In some embodiments, the ITRs are derived from adenovirus, AAV2, and/or AAV5.

In certain embodiments, the vector comprising a template polynucleotide is single stranded AAV (ssAAV). In certain embodiments, the vector comprising a donor template polynucleotide construct is self-complementary AAV (scAAV).

In some embodiments, a vector comprises an attachment site-containing guideRNA (atgRNA), a nicking-guideRNA (ngRNA), and template polynucleotide. In typical embodiments, the vector comprising an attachment site-containing guideRNA (atgRNA), a nicking-guideRNA (ngRNA), and template polynucleotide is recombinant adenovirus, helper dependent adenovirus, AAV, lentivirus, HSV, annelovirus, retrovirus, Doggybone™ DNA (dbDNA), minicircle, plasmid, miniDNA, exosome, fusosome, or nanoplasmid. In preferred embodiments, the vector is capable of localizing to the nucleus. In typical embodiments, the attachment site-containing guideRNA (atgRNA) sequence and the nicking-guideRNA (ngRNA) sequence contain a terminal poly dT.

In some embodiments, a vector comprises an attachment site-containing guideRNA (atgRNA), and donor template. In typical embodiments, the vector comprising an attachment site-containing guideRNA (atgRNA) and donor template is recombinant adenovirus, helper dependent adenovirus, AAV, lentivirus, HSV, annelovirus, retrovirus, Doggybone™ DNA (dbDNA), minicircle, plasmid, miniDNA, exosome, fusosome, or nanoplasmid. In preferred embodiments, the vector is capable of localizing to the nucleus. In typical embodiments, the attachment site-containing guideRNA (atgRNA) sequence contain a terminal poly dT.

In typical embodiments, the template polynucleotide is capable of being integrated into a genomic locus that contains an integrase target recognition site or a recombinase target recognition site.

In certain embodiments, the template polynucleotide comprises at least one of the following: a gene, a gene fragment, an expression cassette, a logic gate system, or any combination thereof. In some embodiments, the template polynucleotide comprises at least one intron or exon.

In typical embodiments, the template polynucleotide further comprises at least one integrase target recognition site or a recombinase target integrase site. In certain embodiments, at least one integrase target recognition site or a recombinase target integrase site is placed within the donor template vector inverted terminal repeat.

### 6.10.4.3 Integrase- or Recombinase-Mediated Self-Circularization of a Subsequence of a Vector Delivered as Part of the Co-Delivery System

In some embodiments, the delivery system (e.g., co-delivery system) includes a vector having a sub-sequence that is capable of self-circularizing to form a self-circular nucleic acid. In some embodiments, the vector comprises a physical portion or region of the vector that is capable of self-circularizing to form a circular construct. As used herein, the term “sub-sequence” refers to a portion of the vector that is capable of self-circularizing, where the sub-sequence is flanked by integration recognition sites or recombinase recognition sites positioned to enable self-circularization. As used herein, the term “self-circular nucleic acid” refers to a double-stranded, circular nucleic acid construct produced as a result of recombination of a cognate pair of integrase or recombinase recognition sites present on the vector. Recombination occurs when the vector is contacted with an integrase or a recombinase under conditions that allow for recombination of the cognate pair of integrase or recombinase recognition sites.

In some embodiments, the sub-sequence of the vector includes a first recombinase recognition site and a second recombinase recognition site, wherein the first and second recombinase recognition sites are capable of being recombined by a recombinase. In some embodiments, the sub-sequence of the vector includes a first recombinase recognition site, a second recombinase recognition site, and a second integration recognition site (e.g., the second integration recognition site is a cognate pair of the first integration recognition site), where the first and second recombinase recognition sites flank the integration recognition site. In such cases, the first recombinase recognition site, the second recombinase recognition, and a recombinase enable the self-circularizing and formation of the circular construct.

In some embodiments, the sub-sequence of the vector includes a third integration recognition site and a fourth integration recognition site, wherein the third and fourth integration recognition sites are a cognate pair. In some embodiments, the subsequence of the vector includes the second integration recognition site, the third integration recognition site, the fourth integration recognition site, where the third and fourth integration recognition sites flank the second integration recognition site (where the second integration recognition site is a cognate pair of the first integration recognition site). In such cases, the third integration recognition site, the fourth integration recognition site, and an integrase enable self-circularization and formation of the circular construct. In such cases, the third integration recognition site and/or the fourth integration recognition sites cannot recombine with the first integration recognition site and/or the second integration recognition site due, in part, to having different central dinucleotides than the first and second integration recognition sites.

In some embodiments where the subsequence includes three or more integration recognition sites, each integration recognition site or each pair of integration recognition is capable of being recognized by a different integrase. In some embodiments where the subsequence includes three or more integration recognition sites, each integration recognition site or each pair of integration recognition comprises a different central dinucleotide.

In some embodiments, self-circularizing is mediated at the integration recognition sites or recombinase recognition sites. In some embodiments, the self-circularizing is mediated by an integrase or a recombinase.

In some embodiments, upon introducing the vector into a cell and after self-circularizing to form the self-circular nucleic acid, the self-circular nucleic acid comprising the second integration recognition site is capable of being integrated into the cell's genome at the target sequence that contains the first integration recognition site.

In some embodiments, following self-circularization, the self-circular nucleic acid comprises one or more additional integration recognition sites that enable integration of an additional nucleic acid cargo. In such cases, the additional nucleic acid cargo includes a sequence that is a cognate pair with one or more of the additional integration recognition sites in the self-circular nucleic acid. For example, integration of the self-circular nucleic acid into the genome of a cell results in integration of the one or more additional integration recognition sites into the genome along with the nucleic acid cargo. The integrated one or more additional integration recognition sites serve as an integration recognition site (beacon) for placing the additional nucleic acid cargo. Upon contacting the cell harboring the integrated nucleic acid cargo and the one or more additional integration recognition sites with an integrase and the second additional nucleic acid cargo that includes a sequence that is an integration cognate to the one or more additional integration recognition sites the additional nucleic acid cargo is integrated into the cell's genome.

In typical embodiments, the self-circularized nucleic acid comprises a DNA cargo. embodiments, the DNA cargo is a gene or gene fragment. In some embodiments the DNA cargo is an expression cassette. In some embodiments, the DNA cargo is a logic gate or logic gate system. The logic gate or logic gate system may be DNA based, RNA based, protein based, or a mix of DNA, RNA, and protein. In some embodiments, the nucleic acid cargo is a genetic, protein, or peptide tag and/or barcode.

### 6.10.4.4 A Second Vector

In some embodiments, the system or methods described herein include a second vector. In some embodiments, where the gene editor polynucleotide encodes a prime editor system comprising a nickase (e.g., any of the Cas proteins or variants thereof (e.g., nickases) and nickases described herein, see Tables 4-8) and a reverse transcriptase (e.g., any of the reverse transcriptase described herein), the second vector comprises a polynucleotide sequence encoding an integrase (e.g., any of the integrases described herein, e.g., as described in Table 10 and also in Yamall et al., Nat. Biotechnol., 2022, doi.org/10.1038/s41587-022-01527-4 and Durrant et al., Nat. Biotechnol., 2022, doi.org/10.1038/s41587-022-01494-w, each of which are herein incorporated by reference in their entireties).

In some embodiments, where the gene editor polynucleotide encodes a prime editor system comprising a nickase and a reverse transcriptase, the second vector comprises a polynucleotide sequence encoding at least a first recombinase. In some embodiments, where the gene editor polynucleotide encodes a prime editor system comprising a nickase, a reverse transcriptase, and an integrase, the second vector comprises a polynucleotide sequence encoding at least a first recombinase. In some embodiments, where the gene editor polynucleotide encodes a prime editor system comprising a nickase, a reverse transcriptase, and an integrase, the second vector comprises a polynucleotide sequence encoding at least a second integrase.

In some embodiments, the second vector includes a template polynucleotide and a sequence that is an integration cognate of an integration recognition site site-specifically incorporated into the genome of a cell. For example, the second vector includes a template polynucleotide and a second integration recognition site that is a cognate pair with the first integration recognition site site-specifically incorporated into the genome of the cell. In such embodiments, the sequence that is an integration cognate (e.g., a second integration recognition site) enables integration of the template polynucleotide or portion thereof when contacted with an integrase and the site-specifically incorporated first integration recognition site.

In some embodiments, the second vector is a vector selected from: adenovirus, AAV, lentivirus, HSV, annelovirus, retrovirus, Doggybone™ DNA (dbDNA), minicircle, plasmid, miniDNA, exosome, fusosome, or nanoplasmid.

In some embodiments, the polynucleotide sequence encoding the prime editor system is encoded on at least two different vectors. In one embodiment, a first vector comprises a polynucleotide sequence encoding a nickase and a second vector comprises a polynucleotide sequence encoding a reverse transcriptase. In such cases, the first vector and second are delivered concurrently.

In some embodiments, the polynucleotide sequence(s) encoding the prime editor system is encoded on at least two (non-contiguous) polynucleotide sequences. In one embodiment, a first polynucleotide sequence encodes a nickase and a second polynucleotide sequence encodes a reverse transcriptase. In such cases, the first vector and second are delivered concurrently (e.g., in a first LNP).

### 6.10.5. Split Lipid Nanoparticles (LNPs)

Also provided herein are methods of co-delivering a system capable of site-specifically integrating at least a first integration recognition site into the genome of a cell, where the method includes delivering to a cell a mixture of a first LNP and a second LNP (“split LNPs”). In one embodiment, the method includes co-delivering to a cell a first gene editor polynucleotide construct and a first attachment site-containing guide RNA (atgRNA) are packaged, and thereby vectorized, within the first LNP, and a second gene editor polynucleotide construct and a second attachment site containing guide RNR (atgRNA) are packaged, and thereby vectorized, within the second LNP, where the first atgRNA and the second atgRNA are an at least first pair of atgRNA. The at least first pair of atgRNAs comprise domains that are capable of guiding the prime editor system to a target sequence. The first atgRNA further includes a first RT template that comprises at least a portion of a first integration recognition site. The second atgRNA further includes a second RT template that comprises at least a portion of the first integration recognition site. The first atgRNA and the second atgRNAs collectively encode the entirety of the first integration recognition site. In such embodiments, the first atgRNA and second atgRNA include at least a 6 bp overlap.

In some embodiments, where the method includes delivering a first LNP (e.g., a first LNP comprising a first gene editor polynucleotide construct and a first atgRNA) and a second LNP (e.g., a second LNP comprising a second gene editor polynucleotide construct and a second atgRNA), the first LNP and the second LNP are mixed prior to delivering to a cell. In some embodiments, the first LNP and the second LNP are mixed at a ratio of first LNP to second LNP of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the first LNP and the second LNP are mixed at a ratio of 1:1.

In some embodiments, a first LNP comprising a first gene editor polynucleotide construct and a first attachment site-containing guide RNA (atgRNA1) comprises a ratio of ratio of gene editor polynucleotide construct (e.g., mRNA) to atgRNA1 of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the first LNP comprises a ratio of mRNA to atgRNA1 of 2:1.

In some embodiments, a second LNP comprising a second gene editor polynucleotide construct and a second attachment site-containing guide RNA (atgRNA2) comprises a ratio of gene editor polynucleotide construct (e.g., mRNA) to atgRNA2 of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the second LNP comprises a ratio of mRNA to atgRNA2 of 2:1.

In some embodiments, where the method includes delivering a first LNP (e.g., a first LNP comprising a first gene editor polynucleotide construct and a first atgRNA) and a second LNP (e.g., a second LNP comprising a second gene editor polynucleotide construct and a second atgRNA), the first LNP and the second LNP are mixed such that the ratio of gene editor polynucleotide construct (e.g., mRNA) to first atgRNA (atgRNA1) to second atgRNA (atgRNA2) is 1:0.25:0.25, 1:0.5:0.5, 1:0.75:0.75, or 1:1:1.

In some embodiments, the method of co-delivering to a cell a mixture of LNPs includes co-delivering three or more LNPs, four or more LNPs, five or more LNPs, six or more LNPs, seven or more LNPs, eight or more LNPs, nine or more LNPs, or ten or more LNPs.

Also provided herein is a system capable of site-specifically integrating at least a first integration recognition site into the genome of a cell, the system comprising: a first gene editor polynucleotide construct and a first attachment site-containing guide RNA (atgRNA) are packaged, and thereby vectorized, within the first LNP, and a second gene editor polynucleotide construct and a second attachment site containing guide RNR (atgRNA) are packaged, and thereby vectorized, within the second LNP, where the first atgRNA and the second atgRNA are an at least first pair of atgRNA. The at least first pair of atgRNAs comprise domains that are capable of guiding the prime editor system to a target sequence. The first atgRNA further includes a first RT template that comprises at least a portion of a first integration recognition site. The second atgRNA further includes a second RT template that comprises at least a portion of the first integration recognition site. The first atgRNA and the second atgRNAs collectively encode the entirety of the first integration recognition site. In such embodiments, the first atgRNA and second atgRNA include at least a 6 bp overlap.

In some embodiments, the system comprises a first LNP (e.g., any of the first LNPs described herein) and a second LNP (e.g., any of the second LNPs described herein) at a ratio of first LNP to second LNP of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the system comprise the first LNP and the second LNP at a ratio of 1:1.

In some embodiments, the system comprises a first LNP having a ratio of a first gene editor polynucleotide construct to a first attachment site-containing guide RNA (atgRNA1) of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the system includes a first LNP having a ratio of mRNA (i.e., mRNA encoding the gene editor protein) to atgRNA1 of 2:1.

In some embodiments, the system comprise a second LNP having a ratio of a second gene editor polynucleotide construct to a second attachment site-containing guide RNA (atgRNA2) of 1:10, 1:9, 1:8, 1:7, 1:6, 1:5, 1:4, 1:3, 1:2, 1:1, 1:0.75, 0.75:1, 2:1, 3:1, 4:1, 5:1, 6:1, 7:1, 8:1, 9:1, or 10:1. In some embodiments, the system includes a second LNP having a ratio of mRNA (i.e., mRNA encoding the gene editor protein) to atgRNA2 of 2:1.

In some embodiments, the system comprises a ratio of gene editor polynucleotide construct (e.g., mRNA encoding the gene editor protein) to first atgRNA (atgRNA1) to second atgRNA (atgRNA2) of 1:0.25:0.25, 1:0.5:0.5, 1:0.75:0.75, or 1:1:1.

In some embodiments, the system comprises a mixture of LNPs comprising three or more LNPs, four or more LNPs, five or more LNPs, six or more LNPs, seven or more LNPs, eight or more LNPs, nine or more LNPs, or ten or more LNPs.

In some embodiments, where a split LNP (e.g., a mixture of two LNPs packaged with different cargo) is being used to site-specifically integrate the at least first integration recognition site into the genome, a vector comprising a template polynucleotide and a sequence that is an integration cognate (i.e., cognate to an integration recognition site site-specifically incorporated into the genome of a cell) can be delivered to the cell concurrently with the split LNPs or after delivery of the split LNPs. For example, after delivering the split LNPs to the cell, a vector that includes a template polynucleotide and a second integration recognition site that is a cognate pair with the first integration recognition site is delivered to the cell. In such embodiments, the sequence that is an integration cognate (e.g., a second integration recognition site) enables integration of the template polynucleotide or portion thereof when contacted with an integrase and the site-specifically incorporated first integration recognition site.

### 6.10.6. Vector Delivery of a Template Polynucleotide

In certain aspects the invention involves vectors, e.g. for delivering or introducing in a cell, but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004, as US 2004-0171156 A1, the contents of which are herein incorporated by reference in their entirety.

Vector delivery, e.g., plasmid, viral delivery: The CRISPR enzyme, for instance a Type V protein such as C2c1 or C2c3, and/or any of the present RNAs, for instance a guide RNA, can be delivered using any suitable vector, e.g., plasmid or viral vectors, such as adeno associated virus (AAV), lentivirus, adenovirus or other viral vector types, or combinations thereof. Effector proteins and one or more guide RNAs can be packaged into one or more vectors, e.g., plasmid or viral vectors. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹¹ particles), more preferably at least about 1×10⁷ particles, more preferably at least about 1×10⁸ particles (e.g., about 1×10⁸-1×10¹¹ particles or about 1×10⁹-1×10¹² particles), and most preferably at least about 1×10¹⁰ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ particles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV (sometimes referred to herein as “vector genomes” or “vg”), from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

The promoter used to drive nucleic acid-targeting effector protein coding nucleic acid molecule expression can include: AAV ITR can serve as a promoter: this is advantageous for eliminating the need for an additional promoter element (which can take up space in the vector). The additional space freed up can be used to drive the expression of additional elements (gRNA, etc.). Also, ITR activity is relatively weaker, so can be used to reduce potential toxicity due to over expression of nucleic acid-targeting effector protein. For ubiquitous expression, can use promoters: CMV, CAG, CBh, PGK, SV40, Ferritin heavy or light chains, etc. For brain or other CNS expression, can use promoters: SynapsinI for all neurons, CaMKIIalpha for excitatory neurons, GAD67 or GAD65 or VGAT for GABAergic neurons, etc. For liver expression, can use Albumin promoter. For lung expression, can use SP-B. For endothelial cells, can use ICAM. For hematopoietic cells can use IFNbeta or CD45. For Osteoblasts can use OG-2.

The promoter used to drive guide RNA can include: Pol III promoters such as U6 or H1 Use of Pol II promoter and intronic cassettes to express guide RNA Adeno Associated Virus (AAV).

Nucleic acid-targeting effector protein and one or more guide RNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific genome modification, the expression of nucleic acid-targeting effector can be driven by a cell-type specific promoter. For example, liver-specific expression might use the Albumin promoter and neuron-specific expression (e.g., for targeting CNS disorders) might use the Synapsin I promoter.

In terms of in vivo delivery, AAV is advantageous over other viral vectors for a couple of reasons: Low toxicity (this may be due to the purification method not requiring ultra centrifugation of cell particles that can activate the immune response) and Low probability of causing insertional mutagenesis because it doesn't integrate into the host genome.

AAV has a packaging limit of 4.5 or 4.75 Kb. This means that nucleic acid-targeting effector protein (such as a Type V protein such as C2c1 or C2c3) as well as a promoter and transcription terminator have to be all fit into the same viral vector. Therefore embodiments of the invention include utilizing homologs of nucleic acid-targeting effector protein (such as a Type V protein such as C2c1 or C2c3) that are shorter.

As to AAV, the AAV can be AAV1, AAV2, AAV5 or any combination thereof. One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The herein promoters and vectors are preferred individually.

Packaging cells are typically used to form virus particles that are capable of infecting a host cell. Such cells include 293 cells, which package adenovirus, and psi2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

Millington-Ward et al. (Molecular Therapy, vol. 19 no. 4, 642-649 April 2011) describes adeno-associated virus (AAV) vectors to deliver an RNA interference (RNAi)-based rhodopsin suppressor and a codon-modified rhodopsin replacement gene resistant to suppression due to nucleotide alterations at degenerate positions over the RNAi target site. An injection of either 6.0×10⁸ vp or 1.8×10¹⁰ vp AAV were subretinally injected into the eyes by Millington-Ward et al. The AAV vectors of Millington-Ward et al. may be applied to the system of the present invention, contemplating a dose of about 2×10¹¹ to about 6×10¹¹ vp administered to a human.

Dalkara et al. (Sci Transl Med 5, 189ra76 (2013)) also relates to in vivo directed evolution to fashion an AAV vector that delivers wild-type versions of defective genes throughout the retina after noninjurious injection into the eyes' vitreous humor. Dalkara describes a 7 mer peptide display library and an AAV library constructed by DNA shuffling of cap genes from AAV1, 2, 4, 5, 6, 8, and 9. The rcAAV libraries and rAAV vectors expressing GFP under a CAG or Rho promoter were packaged and deoxyribonuclease-resistant genomic titers were obtained through quantitative PCR. The libraries were pooled, and two rounds of evolution were performed, each consisting of initial library diversification followed by three in vivo selection steps. In each such step, P30 rho-GFP mice were intravitreally injected with 2 ml of iodixanol-purified, phosphate-buffered saline (PBS)-dialyzed library with a genomic titer of about 1.times.10.sup.12 vg/ml. The AAV vectors of Dalkara et al. may be applied to the nucleic acid-targeting system of the present invention, contemplating a dose of about 1×10¹⁵ to about 1×10¹⁶ vg/ml administered to a human.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SW), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and yr2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. Cells taken from a subject include, but are not limited to, hepatocytes or cells isolated from muscle, the CNS, eye or lung. Immunological cells are also contemplated, such as but not limited to T cells, HSCs, B-cells and NK cells.

Another useful method to deliver proteins, enzymes, and guides comprises transfection of messenger RNA (mRNA). Examples of mRNA delivery methods and compositions that may be utilized in the present disclosure including, for example, PCT/US2014/028330, U.S. Pat. No. 8,822,663B2, NZ700688A, ES2740248T3, EP2755693A4, EP2755986A4, WO2014152940A1, EP3450553B1, BR112016030852A2, and EP3362461A1. Expression of CRISPR systems in particular is described by WO2020014577. Each of these publications are incorporated herein by reference in their entireties. Additional disclosure hereby incorporated by reference can be found in Kowalski et al., “Delivering the Messenger: Advances in Technologies for Therapeutic mRNA Delivery,” Mol Therap., 2019; 27(4): 710-728.

In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences.

In some embodiments, one or more vectors described herein are used to produce a non-human transgenic animal or transgenic plant. In some embodiments, the transgenic animal is a mammal, such as a mouse, rat, or rabbit. In certain embodiments, the organism or subject is a plant. In certain embodiments, the organism or subject or plant is algae. Methods for producing transgenic plants and animals are known in the art, and generally begin with a method of cell transfection, such as described herein.

In one aspect, the invention provides for methods of modifying a target polynucleotide in a prokaryotic or eukaryotic cell, which may be in vivo, ex vivo or in vitro. In some embodiments, the method comprises sampling a cell or population of cells from a human or non-human animal or plant (including micro-algae) and modifying the cell or cells. Culturing may occur at any stage ex vivo. The cell or cells may even be re-introduced into the non-human animal or plant (including micro-algae).

In plants, pathogens are often host-specific. For example, Fusariumn oxysporum f sp. lycopersici causes tomato wilt but attacks only tomato, and F. oxysporum f. dianthii Puccinia graminis f. sp. tritici attacks only wheat. Plants have existing and induced defenses to resist most pathogens. Mutations and recombination events across plant generations lead to genetic variability that gives rise to susceptibility, especially as pathogens reproduce with more frequency than plants. In plants there can be non-host resistance, e.g., the host and pathogen are incompatible. There can also be Horizontal Resistance, e.g., partial resistance against all races of a pathogen, typically controlled by many genes and Vertical Resistance, e.g., complete resistance to some races of a pathogen but not to other races, typically controlled by a few genes. In a Gene-for-Gene level, plants and pathogens evolve together, and the genetic changes in one balance changes in other. Accordingly, using Natural Variability, breeders combine most useful genes for Yield. Quality, Uniformity, Hardiness, Resistance. The sources of resistance genes include native or foreign Varieties, Heirloom Varieties, Wild Plant Relatives, and Induced Mutations, e.g., treating plant material with mutagenic agents. Using the present invention, plant breeders are provided with a new tool to induce mutations. Accordingly, one skilled in the art can analyze the genome of sources of resistance genes, and in Varieties having desired characteristics or traits employ the present invention to induce the rise of resistance genes, with more precision than previous mutagenic agents and hence accelerate and improve plant breeding programs.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

### 6.10.7. Lipid Nanoparticle Delivery

In some embodiments, the delivery system is packaged in one or more LNPs and administered intravenously. In some embodiments, the co-delivery system is packaged in one or more LNPs and administered intrathecally. In some embodiments, the co-delivery system is packaged in one or more LNPs and administered by intracerebral ventricular injection. In some embodiments, the co-delivery system is packaged in one or more LNPs and administered by intracisternal magna administration. In some embodiments, the co-delivery system is packaged in one or more LNPs and administered by intravitreal injection.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). In some embodiments, the LNP formulations are selected from LP01 (Cas No. 1799316-64-5), ALC-0315 (Cas No. 2036272-55-4), and cKK-E12 (Cas No. 1432494-65-9). In some embodiments, the LNP formulation is LP01. In some embodiments, the LNP formulation is ALC-0315. In some embodiment, the LNP formulation is cKK-E12.

In some embodiments, LNP doses range from about 0.1 mg/kg to about 100 mg/kg (or any of the values or subranges therein). In some embodiments, LNP doses is about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, 1.5 mg/kg, about 2 mg/kg, about 2.5 mg/kg, about 3 mg/kg, about 3.5 mg/kg, about 4 mg/kg, about 4.5 mg/kg, about 5 mg/kg, about 6 mg/kg, about 7 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg or more.

In another embodiment, LNP doses of about 0.01 to about 1 mg per kg of body weight administered intravenously are contemplated. Medications to reduce the risk of infusion-related reactions are contemplated, such as dexamethasone, acetaminophen, diphenhydramine or cetirizine, and ranitidine are contemplated. Multiple doses of about 0.3 mg per kilogram every 4 weeks for five doses are also contemplated.

The charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-2200, December 2011). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-2200, December 2011). A dosage of 1 μg/ml of LNP in or associated with the LNP may be contemplated, especially for a formulation containing DLinKC2-DMA.

In some embodiments, the LNP composition comprises one or more one or more ionizable lipids. As used herein, the term “ionizable lipid” has its ordinary meaning in the art and may refer to a lipid comprising one or more charged moieties. In some embodiments, an ionizable lipid may be positively charged or negatively charged. In principle, there are no specific limitations concerning the ionizable lipids of the LNP compositions disclosed herein. In some embodiments, the one or more ionizable lipids are selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanami-ne (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3)-cholest-5-en-3-yloxy]octyl}oxy)-N,N-dimethyl-3-[(9Z,12Z)-octad-eca-9,12-dien-1-yloxy]propan-1-amine (Octyl-CLinDMA), (2R)-2-({8-[(3)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3)-cholest-5-en-3-yloxy]octyl}oxy)-N,N-dimethyl-3-[(9Z,12Z)-octadeca-9,12-dien-1-y loxy]propan-1-amine (Octyl-CLinDMA (2S)). In one embodiment, the ionizable lipid may be selected from, but not limited to, an ionizable lipid described in International Publication Nos. WO2013086354 and WO2013116126.

In some embodiments, the lipid nanoparticle may include one or more (e.g., 1, 2, 3, 4, 5, 6, 7, or 8) cationic and/or ionizable lipids. Such cationic and/or ionizable lipids include, but are not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanami-ne (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 2-({8-[(3.beta.)-cholest-5-en-3-yloxy]octyl}oxy)-N,N-dimethyl-3-[(9Z,12Z)- -octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yl oxy]propan-1-amine (Octyl-CLinDMA (2R)), (2S)-2-({8-[(3pcholest-5-en-3-yloxy]octyl}oxy)-N,N-dimethyl-3-[(9Z-,12Z)-octadeca-9,12-dien-1-yl oxy]propan-1-amine (Octyl-CLinDMA (2S)). N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3-.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl- -ammonium trifluoracetate (“DOSPA”), dioctadecylamidoglycyl carboxyspermine (“DOGS”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic and/or ionizable lipids can be used, such as, e.g., LIPOFECTIN® (including DOTMA and DOPE, available from GIBCO/BRL), and LIPOFECTAMINE® (including DOSPA and DOPE, available from GIBCO/BRL). KL10, KL22, and KL25 are described, for example, in U.S. Pat. No. 8,691,750.

In some embodiments, the LNP composition comprises one or more amino lipids. The terms “amino lipid” and “cationic lipid” are used interchangeably herein to include those lipids and salts thereof having one, two, three, or more fatty acid or fatty alkyl chains and a pH-titratable amino head group (e.g., an alkylamino or dialkylamino head group). In principle, there are no specific limitations concerning the amino lipids of the LNP compositions disclosed herein. The cationic lipid is typically protonated (i.e., positively charged) at a pH below the pKa of the cationic lipid and is substantially neutral at a pH above the pKa. The cationic lipids can also be termed titratable cationic lipids. In some embodiments, the one or more cationic lipids include: a protonatable tertiary amine (e.g., pH-titratable) head group; alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DOTMA, DLinDMA, DLenDMA, .gamma.-DLenDMA, DLin-K-DMA, DLin-K-C2-DMA (also known as DLin-C2K-DMA, XTC2, and C2K), DLin-K-C3-DMA, DLin-K-C4-DMA, DLen-C2K-DMA, y-DLen-C2-DMA, C12-200, cKK-E12, cKK-A12, cKK-O12, DLin-MC2-DMA (also known as MC2), and DLin-MC3-DMA (also known as MC3).

Anionic lipids suitable for use in lipid nanoparticles include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Neutral lipids (including both uncharged and zwitterionic lipids) suitable for use in lipid nanoparticles include, but are not limited to, diacylphosphatidylcholine, diacylphosphatidylethanolamine, ceramide, sphingomyelin, dihydrosphingomyelin, cephalin, sterols (e.g., cholesterol) and cerebrosides. In some embodiments, the lipid nanoparticle comprises cholesterol. Lipids having a variety of acyl chain groups of varying chain length and degree of saturation are available or may be isolated or synthesized by well-known techniques. Additionally, lipids having mixtures of saturated and unsaturated fatty acid chains and cyclic regions can be used. In some embodiments, the neutral lipids used in the disclosure are DOPE, DSPC, DPPC, POPC, or any related phosphatidylcholine. In some embodiments, the neutral lipid may be composed of sphingomyelin, dihydrosphingomyeline, or phospholipids with other head groups, such as serine and inositol.

In some embodiments, amphipathic lipids are included in nanoparticles. Exemplary amphipathic lipids suitable for use in nanoparticles include, but are not limited to, sphingolipids, phospholipids, fatty acids, and amino lipids.

The lipid composition of the pharmaceutical composition may comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular amphipathic lipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural amphipathic lipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the LNP composition comprises one or more phospholipids. In some embodiments, the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16:0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolaminel,2-didocosahexaenoyl-sn-glycero-3-phosphoethanolamine, 1,2-dioleoyl-sn-glycero-3-phospho-rac-(1-glycerol) sodium salt (DOPG), sphingomyelin, and any mixtures thereof.

Other phosphorus-lacking compounds, such as sphingolipids, glycosphingolipid families, diacylglycerols, and .beta.-acyloxyacids, may also be used. Additionally, such amphipathic lipids can be readily mixed with other lipids, such as triglycerides and sterols.

In some embodiments, the LNP composition comprises one or more helper lipids. The term “helper lipid” as used herein refers to lipids that enhance transfection (e.g., transfection of an LNP comprising an mRNA that encodes a site-directed endonuclease, such as a SpCas9 polypeptide). In principle, there are no specific limitations concerning the helper lipids of the LNP compositions disclosed herein. Without being bound to any particular theory, it is believed that the mechanism by which the helper lipid enhances transfection includes enhancing particle stability. In some embodiments, the helper lipid enhances membrane fusogenicity. Generally, the helper lipid of the LNP compositions disclosure herein can be any helper lipid known in the art. Non-limiting examples of helper lipids suitable for the compositions and methods include steroids, sterols, and alkyl resorcinols. Particularly helper lipids suitable for use in the present disclosure include, but are not limited to, saturated phosphatidylcholine (PC) such as distearoyl-PC (DSPC) and dipalymitoyl-PC (DPPC), dioleoylphosphatidylethanolamine (DOPE), 1,2-dilinoleoyl-sn-glycero-3-phosphocholine (DLPC), cholesterol, 5-heptadecylresorcinol, and cholesterol hemisuccinate. In some embodiments, the helper lipid of the LNP composition includes cholesterol.

In some embodiments, the LNP composition comprises one or more structural lipids. As used herein, the term “structural lipid” refers to sterols and also to lipids containing sterol moieties. Without being bound to any particular theory, it is believed that the incorporation of structural lipids into the LNPs mitigates aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In some embodiments, the structural lipid is cholesterol. In certain embodiments, the structural lipid is an analog of cholesterol.

The lipid component of a lipid nanoparticle composition may include one or more molecules comprising polyethylene glycol, such as PEG or PEG-modified lipids. In some embodiments, the LNP composition disclosed herein comprise one or more polyethylene glycol (PEG) lipid. The term “PEG-lipid” refers to polyethylene glycol (PEG)-modified lipids. Such lipids are also referred to as PEGylated lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines For example, a PEG lipid can be PEG-c-DOMG, PEG-DMG, PEG-DLPE, PEG-DMPE, PEG-DPPC, or a PEG-DSPE lipid. In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the lipid moiety of the PEG-lipids includes those having lengths of from about C.sub.14 to about C.sub.22, preferably from about C.sub.14 to about C.sub.16. In some embodiments, a PEG moiety, for example a mPEG-NH.sub.2, has a size of about 1000, 2000, 5000, 10,000, 15,000 or 20,000 daltons. In some embodiment, the PEG-lipid is PEG2k-DMG. In some embodiments, the one or more PEG lipids of the LNP composition comprises PEG-DMPE. In some embodiments, the one or more PEG lipids of the LNP composition comprises PEG-DMG.

In some embodiments, the ratio between the lipid components and the nucleic acid molecules of the LNP composition, e.g., the weight ratio, is sufficient for (i) formation of LNPs with desired characteristics, e.g., size, charge, and (ii) delivery of a sufficient dose of nucleic acid at a dose of the lipid component(s) that is tolerable for in vivo administration as readily ascertained by one of skill in the art.

In certain embodiments, it is desirable to target a nanoparticle, e.g., a lipid nanoparticle, using a targeting moiety that is specific to a cell type and/or tissue type. In some embodiments, a nanoparticle may be targeted to a particular cell, tissue, and/or organ using a targeting moiety. In particular embodiments, a nanoparticle comprises a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42(5):439-62, 2003 and Abra et al., J. Liposome Res. 12:1-3, 2002.

In some embodiments, a lipid nanoparticle (e.g., a liposome) may include a surface coating of hydrophilic polymer chains, such as polyethylene glycol (PEG) chains (see, e.g., Allen et al., Biochimica et Biophysica Acta 1237: 99-108, 1995; DeFrees et al., Journal of the American Chemistry Society 118: 6101-6104, 1996; Blume et al., Biochimica et Biophysica Acta 1149: 180-184,1993; Klibanov et al., Journal of Liposome Research 2: 321-334, 1992; U.S. Pat. No. 5,013,556; Zalipsky, Bioconjugate Chemistry 4: 296-299, 1993; Zalipsky, FEBS Letters 353: 71-74, 1994; Zalipsky, in Stealth Liposomes Chapter 9 (Lasic and Martin, Eds) CRC Press, Boca Raton Fla., 1995). In one approach, a targeting moiety for targeting the lipid nanoparticle is linked to the polar head group of lipids forming the nanoparticle. In another approach, the targeting moiety is attached to the distal ends of the PEG chains forming the hydrophilic polymer coating (see, e.g., Klibanov et al., Journal of Liposome Research 2: 321-334, 1992; Kirpotin et al., FEBS Letters 388: 115-118, 1996).

Standard methods for coupling the targeting moiety or moieties may be used. For example, phosphatidylethanolamine, which can be activated for attachment of targeting moieties, or derivatized lipophilic compounds, such as lipid-derivatized bleomycin, can be used. Antibody-targeted liposomes can be constructed using, for instance, liposomes that incorporate protein A (see, e.g., Renneisen et al., J. Bio. Chem., 265:16337-16342, 1990 and Leonetti et al., Proc. Natl. Acad. Sci. (USA), 87:2448-2451, 1990). Other examples of antibody conjugation are disclosed in U.S. Pat. No. 6,027,726. Examples of targeting moieties can also include other polypeptides that are specific to cellular components, including antigens associated with neoplasms or tumors. Polypeptides used as targeting moieties can be attached to the liposomes via covalent bonds (see, for example Heath, Covalent Attachment of Proteins to Liposomes, 149 Methods in Enzymology 111-119 (Academic Press, Inc. 1987)). Other targeting methods include the biotin-avidin system.

In some embodiments, a lipid nanoparticle includes a targeting moiety that targets the lipid nanoparticle to a cell including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the targeting moiety targets the lipid nanoparticle to a hepatocyte.

The lipid nanoparticles described herein may be lipidoid-based. The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat. Biotechnol. 2008 26:561-569; Love et al., Proc Natl Acad Sci USA. 2010 107:1864-1869; Siegwart et al., Proc Natl Acad Sci USA. 2011 108:12996-3001).

The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see e.g., Akinc et al., Mol Ther. 2009 17:872-879), use of lipidoid oligonucleotides to deliver the formulation to other cells types including, but not limited to, endothelial cells, myeloid cells, and muscle cells may not be similarly size-limited.

In one aspect, effective delivery to myeloid cells, such as monocytes, lipidoid formulations may have a similar component molar ratio. Different ratios of lipidoids and other components including, but not limited to, a neutral lipid (e.g., diacylphosphatidylcholine), cholesterol, a PEGylated lipid (e.g., PEG-DMPE), and a fatty acid (e.g., an omega-3 fatty acid) may be used to optimize the formulation of the mRNA or system for delivery to different cell types including, but not limited to, hepatocytes, myeloid cells, muscle cells, etc. Exemplary lipidoids include, but are not limited to, DLin-DMA, DLin-K-DMA, DLin-KC2-DMA, 98N12-5, C12-200 (including variants and derivatives), DLin-MC3-DMA and analogs thereof. The use of lipidoid formulations for the localized delivery of nucleic acids to cells (such as, but not limited to, adipose cells and muscle cells) via either subcutaneous or intramuscular delivery, may also not require all of the formulation components which may be required for systemic delivery, and as such may comprise the lipidoid and the mRNA or system.

According to the present disclosure, a system described herein may be formulated by mixing the mRNA or system, or individual components of the system, with the lipidoid at a set ratio prior to addition to cells. In vivo formulations may require the addition of extra ingredients to facilitate circulation throughout the body. After formation of the particle, a system or individual components of a system is added and allowed to integrate with the complex. The encapsulation efficiency is determined using a standard dye exclusion assays.

In vivo delivery of systems may be affected by many parameters, including, but not limited to, the formulation composition, nature of particle PEGylation, degree of loading, oligonucleotide to lipid ratio, and biophysical parameters such as particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010)), C12-200 (including derivatives and variants), MD1, DLin-DMA, DLin-K-DMA, DLin-KC2-DMA and DLin-MC3-DMA can be tested for in vivo activity. The lipidoid referred to herein as “98N12-5” is disclosed by Akinc et al., Mol Ther. 2009 17:872-879). The lipidoid referred to herein as “C12-200” is disclosed by Love et al., Proc Natl Acad Sci USA. 2010 107:1864-1869 and Liu and Huang, Molecular Therapy. 2010 669-670.

The LNPs of the present disclosure, in which a nucleic acid is entrapped within the lipid portion of the particle and is protected from degradation, can be formed by any method known in the art including, but not limited to, a continuous mixing method, a direct dilution process, and an in-line dilution process. Additional techniques and methods suitable for the preparation of the LNPs described herein include coacervation, microemulsions, supercritical fluid technologies, phase-inversion temperature (PIT) techniques.

In some embodiments, the LNPs used herein are produced via a continuous mixing method, e.g., a process that includes providing an aqueous solution a nucleic acid described herein in a first reservoir, providing an organic lipid solution in a second reservoir (wherein the lipids present in the organic lipid solution are solubilized in an organic solvent, e.g., a lower alkanol such as ethanol), and mixing the aqueous solution with the organic lipid solution such that the organic lipid solution mixes with the aqueous solution so as to substantially instantaneously produce a lipid vesicle (e.g., liposome) encapsulating the nucleic acid molecule within the lipid vesicle. This process and the apparatus for carrying out this process are known in the art. More information in this regard can be found in, for example, U.S. Patent Publication No. 20040142025. The action of continuously introducing lipid and buffer solutions into a mixing environment, such as in a mixing chamber, causes a continuous dilution of the lipid solution with the buffer solution, thereby producing a lipid vesicle substantially instantaneously upon mixing. By mixing the aqueous solution comprising a nucleic acid molecule with the organic lipid solution, the organic lipid solution undergoes a continuous stepwise dilution in the presence of the buffer solution (e.g., aqueous solution) to produce a nucleic acid-lipid particle.

In some embodiments, the LNPs used herein are produced via a direct dilution process that includes forming a lipid vesicle (e.g., liposome) solution and immediately and directly introducing the lipid vesicle solution into a collection vessel containing a controlled amount of dilution buffer. In some embodiments, the collection vessel includes one or more elements configured to stir the contents of the collection vessel to facilitate dilution. In some embodiments, the amount of dilution buffer present in the collection vessel is substantially equal to the volume of lipid vesicle solution introduced thereto.

In some embodiments, the LNPs are produced via an in-line dilution process in which a third reservoir containing dilution buffer is fluidly coupled to a second mixing region. In these embodiments, the lipid vesicle (e.g., liposome) solution formed in a first mixing region is immediately and directly mixed with dilution buffer in the second mixing region. These processes and the apparatuses for carrying out direct dilution and in-line dilution processes are known in the art. More information in this regard can be found in, for example, U.S. Patent Publication No. 20070042031.

### 6.11. Additional Delivery Modalities

This disclosure is not limited to systems and methods described herein. Any delivery method that is capable of delivering the systems described herein can be used as long as it is capable of site-specifically integrating a template polynucleotide into the genome of a cell.

### 6.12. Genes and Targets

This disclosure provides compositions, systems and methods for correcting or replacing genes or gene fragments (including introns or exons) or inserting genes in new locations. In certain embodiments, such a method comprises recombination or integration into a safe harbor site (SHS). A frequently used human SHS is the AAVS1 site on chromosome 19q, initially identified as a site for recurrent adeno-associated virus insertion. Another locus comprises the human homolog of the murine Rosa26 locus. Yet another SHS comprises the human H11 locus on chromosome 22. In some cases, a complete gene may be prohibitively large and replacement of an entire gene impractical. In certain embodiments, a method of the disclosure comprises recombining corrective gene fragments into a defective locus.

The methods and compositions can be used to target, without limitation, stem cells for example induced pluripotent stem cells (iPSCs), HSCs, HSPCs, mesenchymal stem cells, or neuronal stem cells and cells at various stages of differentiation. In certain embodiments, methods and compositions of the disclosure are adapted to target organoids, including patient derived organoids.

In certain embodiments, methods and compositions of the disclosure are adapted to treat muscle cells, not limited to cardiomyocytes for Duchene Muscular Dystrophy (DMD). The dystrophin gene is the largest gene in the human genome, spanning ˜2.3 Mb of DNA. DMD is composed of 79 exons resulting in a 14-kb full-length mRNA. Common mutations include mutations that disrupt the reading frame of generate a premature stop codon. An aspect of DMD that lends it to gene editing as a therapeutic approach is the modular structure of the dystrophin protein. Redundancy in the central rod domain permits the deletion of internal segments of the gene that may harbor loss-of-function mutations, thereby restoring the open reading frame (ORFs). In some embodiments, the methods and systems described herein are used to treat DMD by site-specifically integrating in the genome a polynucleotide template that repairs or replaces all or a portion of the defective DMD gene.

The following are non-limiting diseases that may be treated utilizing the methods and compositions of the present disclosure:

### Inherited Retinal Diseases:

1. Stargardt Disease (ABCA4)
2. Leber congenital amaurosis 10 (CEP290)
3. X linked Retinitis Pigmentosa (RPGR)
4. Autosomal Dominant Retinitis Pigmentosa (RHO)

### Liver Diseases:

5. Wilson's disease (ATP7B)
6. Alpha-1 antitrypsin (SERPINA1)

### Intellectual Disabilities:

7. Rett Syndrome (MECP2)
8. SYNGAP1-ID (SYNGAP1)
9. CDKL5 deficiency disorder (CDKL5)

### Peripheral Neuropathies:

10. Charcot-Marie-Tooth 2A (MFN2)

### Lung Diseases:

11. Cystic Fibrosis (CFTR)
12. Alpha-1 Antitrypsin (SERPINA1)
   Autoimmune diseases:
13. IgA Nephropathy (Berger's disease)
14. Anti-Neutrophil Cytoplasmic Antibody (ANCA) Vasculitis
15. Systemic Lupus Erythematosus (SLE)/Lupus Nephritis (LN)
16. Membranous Nephropathy (MN)
17. C3 glomerulonephritis (C3GN)
18. Minimal Change Disease

### Blood Disorders:

19. Sickle Cell
20. Hemophilia
21. Factor VIII or
22. Factor IX
23. Ornithine transcarbamylase deficiency (OTCD)
24. Homocystinuria (HCU)
25. Phenylketonuria (PKU)

### Cancer

26. Prostate cancer
27. Renal cell cancer
28. Thyroid cancer

### Cardiovascular Disorders:

29. Atherosclerotic cardiovascular disease (ASCVD)

### Other Disorders:

30. Hemochromatosis

### Other Immunological Disorders:

ANCA Vasculitis, Myasthenia Gravis (MG), Systemic sclerosis (SS), Idiopathic thrombocytopenic purpura (ITP), Multiple Sclerosis (MS), Guillain-Barre syndrome (GBS), Chronic Inflammatory Demyelinating polyradiculoneuropathy (CIDP), Pemphigus Vulgaris (PV), Bullous Pemphigoid (BP), Autoimmune Hepatitis (AIH), Autoimmune Hemolytic Anemia (AIHA), Dermatomyositis (DM), Polymyositis (PM), Scleroderma, Neuromyelitis optica spectrum disorders (NMOSD), Myelin oligodendrocyte glycoprotein antibody associated disease (MOGAD), Churg-Strauss syndrome/eosinophilic granulomatosis with polyangiitis (EGPA), CREST syndrome/limited cutaneous systemic sclerosis, Devic's disease/neuromyelitis optica (NMO), Granulomatosis with polyangiitis (GPA)/Wegener's granulomatosis, Henoch-Schonlein purpura/IgA vasculitis, autoimmune thrombocytopenic purpura/autoimmune thrombocytopenia, or Transverse Myelitis™.

CFTR (cystic fibrosis transmembrane conductance regulator). The most common cystic fibrosis (CF) mutation F508del removes a single amino acid. In some embodiments, recombining human CFTR into an SHS of a cell that expresses CFTR F508del is a corrective treatment path. In some embodiments, the methods and systems described herein are used to CF by site-specifically integrating in the genome a polynucleotide template that corrects the mutation causing CF. Proposed validation is detection of persistent CFTR mRNA and protein expression in transduced cells.

Sickle cell disease (SCD) is caused by mutation of a specific amino acid—valine to glutamic acid at amino acid position 6. In some embodiments, SCD is corrected by recombination of the HBB gene into a safe harbor site (SHS) and by demonstrating correction in a proportion of target cells that is high enough to produce a substantial benefit. In some embodiments, the methods and systems described herein are used to sickle cell disease by site-specifically integrating in the genome a polynucleotide template that corrects the mutation causing the disease. In some embodiments, validation is detection of persistent HBB mRNA and protein expression in transduced cells.

DMD—Duchenne Muscular Dystrophy. The dystrophin gene is the largest gene in the human genome, spanning ˜2.3 Mb of DNA. DMD is composed of 79 exons resulting in a 14-kb full-length mRNA. Common mutations include mutations that disrupt the reading frame of generate a premature stop codon. An aspect of DMD that lends it to gene editing as a therapeutic approach is the modular structure of the dystrophin protein. Redundancy in the central rod domain permits the deletion of internal segments of the gene that may harbor loss-of-function mutations, thereby restoring the open reading frame (ORFs).

In some embodiments, recombination will be into safe harbor sites (SHS). A frequently used human SHS is the AAVS1 site on chromosome 19q, initially identified as a site for recurrent adeno-associated virus insertion. In some embodiments, the site is the human homolog of the e murine Rosa26 locus (pubmed.ncbi.nlm.nih.gov/18037879). In some embodiments, the site is the human H11 locus on chromosome 22. Proposed target cells for recombination include stem cells for example induced pluripotent stem cells (iPSCs) and cells at various stages of differentiation. In some cases, a complete gene may be prohibitively large and replacement of an entire gene impractical. In such instances, rescuing mutants by recombining in corrected gene fragments with the methods and systems described herein is a corrective option.

In some embodiments, correcting mutations in exon 44 (or 51) by recombining in a corrective coding sequence downstream of exon 43 (or 50), using the methods and systems described herein is a corrective option. Proposed validation is detection of persistent DMD mRNA and protein expression in transduced cells.

F8 (Factor VIII). A large proportion of severe hemophilia A patients harbor one of two types of chromosomal inversions in the FVIII gene. The recombinase technology and methods described herein are well suited to correcting such inversions (and other mutations) by recombining of the FVIII gene into a SHS.

In some embodiments, correcting factor VIII deficiency by recombining the FVIII gene into an SHS is a corrective path. In some embodiments, the methods and systems described herein are used to correct factor VIII deficiency by site-specifically integrating in the genome a polynucleotide template that corrects the mutation causing the FIX deficiency. Proposed validation is detection of persistent FVIII mRNA and protein expression in transduced cells.

Factor 9 (Factor IX) Hemophilia B, also called factor IX (FIX) deficiency is a genetic disorder caused by missing or defective factor IX, a clotting protein.

In some embodiments, the methods and systems described herein are used to correct factor IX deficiency by site-specifically integrating in the genome a polynucleotide template that corrects the mutation causing the FIX deficiency. Proposed validation is detection of persistent FiX mRNA and protein expression in transduced cells.

Ornithine transcarbamylase deficiency (OTCD). Omithine transcarbamylase deficiency is a rare genetic condition that causes ammonia to build up in the blood. The condition—more commonly called OTC deficiency—is more common in boys than girls and tends to be more severe when symptoms emerge shortly after birth.

In some embodiments, the methods and systems described herein are used to correct OTC deficiency by site-specifically integrating in the genome a polynucleotide template that corrects the mutation causing the OTC deficiency or integrates a polynucleotide encoding a functional ornithine transcarbamylase enzyme. Proposed validation is detection of persistent OTC mRNA and protein expression in transduced cells.

Phenylketonuria, also called PKU, is a rare inherited disorder that causes an amino acid called phenylalanine to build up in the body. PKU is caused by a change in the phenylalanine hydroxylase (PAH) gene. This gene helps create the enzyme needed to break down phenylalanine.

In some embodiments, the methods and systems described herein are used to correct PKU by site-specifically integrating in the genome a polynucleotide template that corrects the mutation causing the PKU deficiency or integrates a polynucleotide encoding a functional phenylalanine hydroxylase (PAH) gene. Proposed validation is detection of persistent PAH mRNA and protein expression in transduced cells.

Homocystinuria (HCU). Homocystinuria is elevation of the amino acid, homocysteine (protein building block coming from our diet) in the urine or blood. Common causes of HCU include: problems with the enzyme cystathionine beta synthase (CBS), which converts homocysteine to the amino acid cystathionine (which then becomes cysteine) and needs the vitamin B6 (pyridoxine); and problems with converting homocysteine to the amino acid methionine.

In some embodiments, the methods and systems described herein are used to correct HCU by site-specifically integrating in the genome a polynucleotide template that corrects the mutation causing the HCU or integrates a polynucleotide encoding a functional copy of a gene (e.g., CBS) able to reduce or prevent buildup of homocysteine in the urine. Proposed validation is detection of persistent CBS mRNA and protein expression in transduced cells.

Hemochromatosis. Hemochromatosis a disorder in which the body can build up too much iron in the skin, heart, liver, pancreas, pituitary gland, and joints. Too much iron can lead to life-threatening conditions, such as liver disease, heart problems and diabetes. Hereditary hemochromatosis is most commonly caused by certain variants in the HFE gene. The two common disease-related mutations are C282Y and H63D.

In some embodiments, the methods and systems described herein are used to correct hemochromatosis by site-specific integrating in the genome a polynucleotide template that corrects the mutation causing hemochromatosis or integrates a polynucleotide encoding a functional HFE protein. Proposed validation is detection of persistent HFE mRNA and protein expression in transduced cells.

Atherosclerotic cardiovascular disease (ASCVD). Atherosclerotic cardiovascular disease (ASCVD) is caused by plaque buildup in arterial walls and refers to conditions that include, for example, coronary heart disease (CHD) (such as myocardial infarction, angina, and coronary artery stenosis), cerebrovascular disease (such as a transient ischemic attack, ischemic stroke, and carotid artery stenosis), peripheral artery disease (such as claudication), and aortic atherosclerotic disease (such as abdominal aortic aneurysm and descending thoracic aneurysm). In some examples, ASCVD is caused by a change in ApoA-1 gene.

In some embodiments, the methods and systems described herein are used to correct ASCVD by site-specifically integrating in the genome a polynucleotide template that corrects the mutation causing the ASCVD or integrates a polynucleotide encoding a functional ApoA-1. Proposed validation is detection of persistent ApoA-1 mRNA and protein expression in transduced cells.

IgA Nephropathy (Berger's disease). IgA nephropathy, also known as Berger's disease, is a kidney/autoimmune disease that occurs when an antibody called immunoglobulin A (IgA) builds up in the kidneys.

In some embodiments, the methods and systems described herein are used to treat Berger's disease by administering to a patient an iPSC-derived Natural Killer cell that includes a polynucleotide site-specifically integrated in the genome of the cell using the methods described herein. Upon administering the iPSC-NK cell to the patient, the iPSC-NK cell is capable of removing native cells (e.g., B cells) that are responsible, at least in part, for the symptoms of Berger's disease.

Anti-Neutrophil Cytoplasmic Antibody (ANCA) Vasculitis. ANCA vasculitis is an autoimmune disease affecting small blood vessels in the body. It is caused by autoantibodies called ANCAs, or Anti-Neutrophilic Cytoplasmic Autoantibodies. ANCAs target and attack a certain kind of white blood cells called neutrophils.

In some embodiments, the methods and systems described herein are used to treat ANCA vasculitis by administering to a patient an iPSC-derived Natural Killer cell that includes a polynucleotide site-specifically integrated in the genome of the cell using the methods described herein. Upon administering the iPSC-NK cell to the patient, the iPSC-NK cell is capable of removing native cells (e.g., B cells) that are responsible, at least in part, for the symptoms of ANCA vasculitis.

Systemic Lupus Erythematosus (SLE)/Lupus Nephritis (LN). Lupus is an autoimmune—a disorder in which the body's immune system attacks the body's own cells and organs.

In some embodiments, the methods and systems described herein are used to treat SLE/LN by administering to a patient an iPSC-derived Natural Killer cell that includes a polynucleotide site-specifically integrated in the genome of the cell using the methods described herein. Upon administering the iPSC-NK cell to the patient, the iPSC-NK cell is capable of removing native cells (e.g., B cells) that are responsible, at least in part, for the symptoms of SLE/LN.

Membranous Nephropathy (MN). MN is a kidney disease that affects the filters (glomeruli) of the kidney and can cause protein in the urine, as well as decreased kidney function and swelling. It can sometimes be called membranous glomerulopathy as well (these terms can be used interchangeably and mean the same thing).

In some embodiments, the methods and systems described herein are used to treat MN by administering to a patient an iPSC-derived Natural Killer cell that includes a polynucleotide site-specifically integrated in the genome of the cell using the methods described herein. Upon administering the iPSC-NK cell to the patient, the iPSC-NK cell is capable of removing native cells (e.g., B cells) that are responsible, at least in part, for the symptoms of MN.

C3 glomerulonephritis (C3GN). C3 glomerulopathy is a group of related conditions that cause the kidneys to malfunction. The major features of C3 glomerulopathy include high levels of protein in the urine (proteinuria), blood in the urine (hematuria), reduced amounts of urine, low levels of protein in the blood, and swelling in many areas of the body. Affected individuals may have particularly low levels of a protein called complement component 3 (or C3) in the blood.

In some embodiments, the methods and systems described herein are used to treat C3 glomerulopathy by administering to a patient an iPSC-derived Natural Killer cell that includes a polynucleotide site-specifically integrated in the genome of the cell using the methods described herein. Upon administering the iPSC-NK cell to the patient, the iPSC-NK cell is capable of removing native cells (e.g., B cells) that are responsible, at least in part, for the symptoms of C3 glomerulopathy.

Minimal Change Disease (MCD). Minimal change disease (MCD) is a condition that damages the blood vessels in kidneys, which can affect how well kidneys work. Minimal change disease (MCD) is one of the most common causes of idiopathic nephrotic syndrome in children. It accounts for 70% to 90% of children that present with nephrotic syndrome who are older than one year old as opposed to 10-15% of adults who present with nephrotic syndrome. The clinical signs of minimal change disease are proteinuria, edema, weight gain, and hypoalbuminemia.

In some embodiments, the methods and systems described herein are used to treat MCD by administering to a patient an iPSC-derived Natural Killer cell that includes a polynucleotide site-specifically integrated in the genome of the cell using the methods described herein. Upon administering the iPSC-NK cell to the patient, the iPSC-NK cell is capable of removing native cells (e.g., B cells) that are responsible, at least in part, for the symptoms of MCD.

### 6.13. Methods of Treatment

In another aspect, methods of treatment are presented. The method comprises administering an effective amount of the pharmaceutical composition comprising the nucleic acid construct or vectorized nucleic acid construct described above to a patient in need thereof. In some embodiments, the system (e.g., any of the systems described herein) are delivered to a cell ex vivo and the cell is then administered to the subject. In some embodiments, the systems (e.g., any of the systems described herein) are delivered to a patient, thereby delivering to a cell in vivo.

DNA or RNA viral vectors can be administered directly to patients (in vivo) or they can be used to treat cells in vitro, and the modified cells may optionally be administered to patients (ex vivo). Conventional viral based systems to be used herein could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In some embodiments, the co-delivery system described herein (e.g., a gene editor construct packaged in a LNP and a donor template packaged in a vector) is administered intravenously. In some embodiments, the co-delivery system described herein (e.g., a gene editor construct packaged in a LNP and a donor template packaged in a vector) is administered intrathecally. In some embodiments, the co-delivery system described herein (e.g., a gene editor construct packaged in a LNP and a donor template packaged in a vector) is administered by intracerebral ventricular injection. In some embodiments, the co-delivery system described herein (e.g., a gene editor construct packaged in a LNP and a donor template packaged in a vector) is administered by intracisternal magna administration. In some embodiments, the co-delivery system described herein (e.g., a gene editor construct packaged in a LNP and a donor template packaged in a vector) is administered by intravitreal injection.

Methods of non-viral delivery of the donor DNA template described herein include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

### 6.13.1. mRNA Delivery

Another useful method to deliver proteins, enzymes, and guides comprises transfection of messenger RNA (mRNA). Examples of mRNA delivery methods and compositions that may be utilized in the present disclosure including, for example, PCT/US2014/028330, U.S. Pat. No. 8,822,663B2, NZ700688A, ES2740248T3, EP2755693A4, EP2755986A4, WO2014152940A1, EP3450553B1, BR112016030852A2, and EP3362461A1. Expression of CRISPR systems in particular is described by WO2020014577. Each of these publications are incorporated herein by reference in their entireties. Additional disclosure hereby incorporated by reference can be found in Kowalski et al., “Delivering the Messenger: Advances in Technologies for Therapeutic mRNA Delivery,” Mol Therap., 2019; 27(4): 710-728.

### 7. EXAMPLES

### 7.1. Example 1: Delivery of Gene Editor Polynucleotide Sequence Packaged in LNP and Donor Template Packaged in AAV

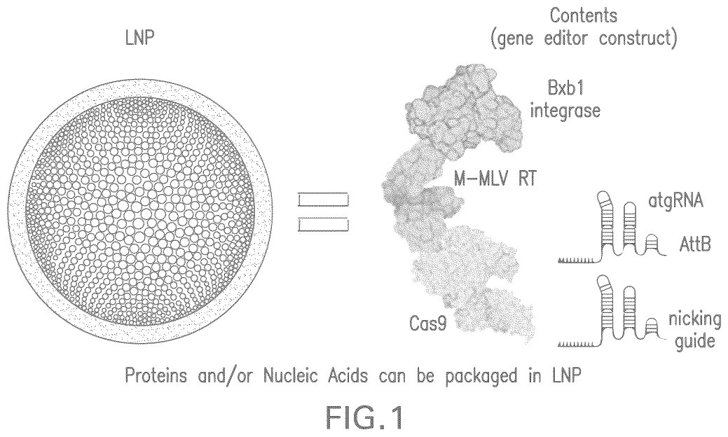

A gene editor polynucleotide construct is packaged into a LNP (FIG. 1), wherein the gene editor polynucleotide sequence comprises a polynucleotide sequence encoding a prime editor protein linked to an integrase via peptide linker a polynucleotide sequence encoding an attachment site-containing guide RNA (atgRNA); a polynucleotide sequence encoding a nickase guide RNA (ngRNA).

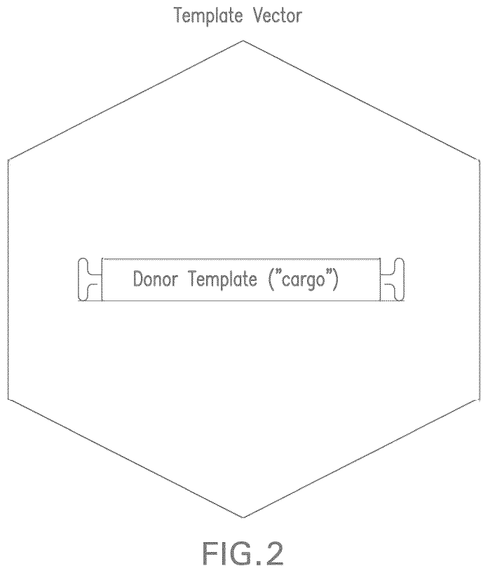

A donor template polynucleotide construct is packaged in an AAV vector (FIG. 2).

Co-administration of the gene editor construct packaged LNP and the donor template packaged AAV co-delivers the gene editor construct to a cell cytoplasm and the donor template to a cell nucleus. By use of programmable genome editing to place integrase landing site at a desired location in the genome, the direct activity of the associated integrase to the specific genomic site is guided. Gene editor construct expression, with template co-delivery, results in integration of template “cargo” at a precisely defined target location.

### 7.2. Example 2: Delivery of Gene Editor Polynucleotide Sequence Packaged in LNP and Donor Template Capable of Self-Circularization Packaged in AAV

A gene editor polynucleotide construct is packaged into a LNP (FIG. 1), wherein the gene editor polynucleotide sequence comprises a polynucleotide sequence encoding a prime editor protein linked to an integrase via peptide linker a polynucleotide sequence encoding an attachment site-containing guide RNA (atgRNA); a polynucleotide sequence encoding a nickase guide RNA (ngRNA).

A donor template polynucleotide construct is packaged in an AAV vector (FIG. 2).

Co-administration of the gene editor construct packaged LNP and the donor template packaged AAV co-delivers the gene editor construct to a cell cytoplasm and the donor template to a cell nucleus. Integrase-mediated self-circularization of donor template occurs at integration target recognition sites within the AAV genome (FIG. 3). By use of programmable genome editing to place an orthogonal integrase landing site (i.e., distinct att site from att sites used for self-circularization) at a desired location in the genome, the direct activity of the associated integrase to the specific genomic site is guided. Gene editor construct expression, with template co-delivery and integrase-mediated circularization of template, results in integration of template “cargo” at a precisely defined target location.

### 7.3. Example 3: Delivery of Gene Editor Polynucleotide Sequence Packaged in LNP and atgRNA, ngRNA, and Donor Template Co-Packaged in AAV

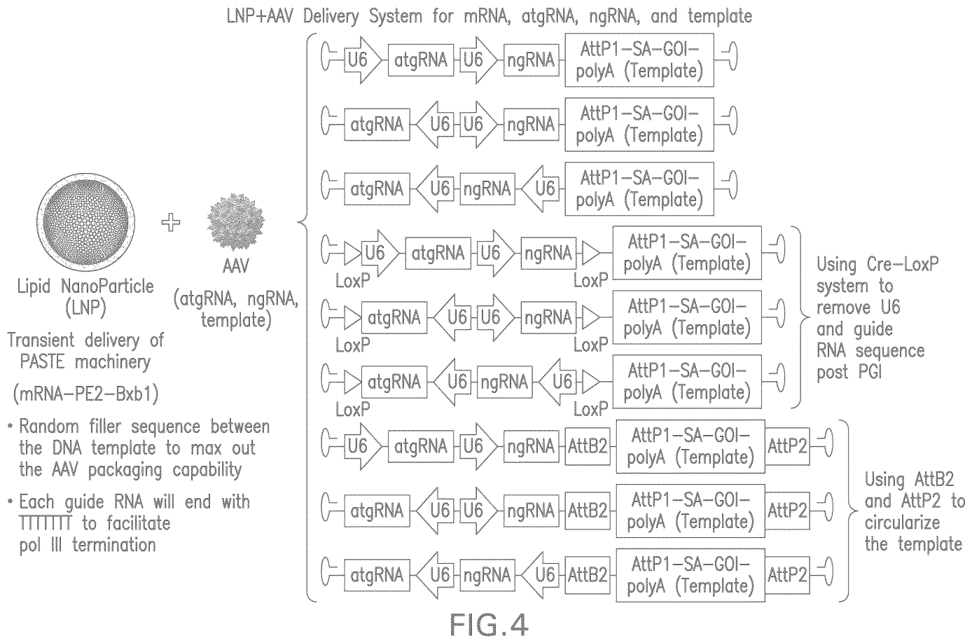

A gene editor polynucleotide construct is packaged into a LNP (FIG. 4), wherein the gene editor polynucleotide sequence comprises a polynucleotide sequence encoding a prime editor protein linked to an integrase via peptide linker.

A polynucleotide sequence encoding an attachment site-containing guide RNA (atgRNA), a polynucleotide sequence encoding a nicking guide RNA (ngRNA), and donor template are packaged in an AAV vector (FIG. 4).

Co-administration of the gene editor construct packaged LNP and the atgRNA, ngRNA, donor template packaged AAV co-delivers the gene editor construct to a cell. Integrase-mediated self-circularization of donor template occurs at integration target recognition sites within the AAV genome (FIG. 3). By use of programmable genome editing to place an orthogonal integrase landing site (i.e., distinct att site from att sites used for self-circularization) at a desired location in the genome, the direct activity of the associated integrase to the specific genomic site is guided. Gene editor construct expression, with atgRNA, ngRNA, and template co-delivery and integrase-mediated circularization of template, results in integration of template “cargo” at a precisely defined target location.

### 7.4. Example 4: Delivery of Gene Editor Polynucleotide Sequence and ngRNA Packaged in LNP and atgRNA and Donor Template Co-Packaged in AAV

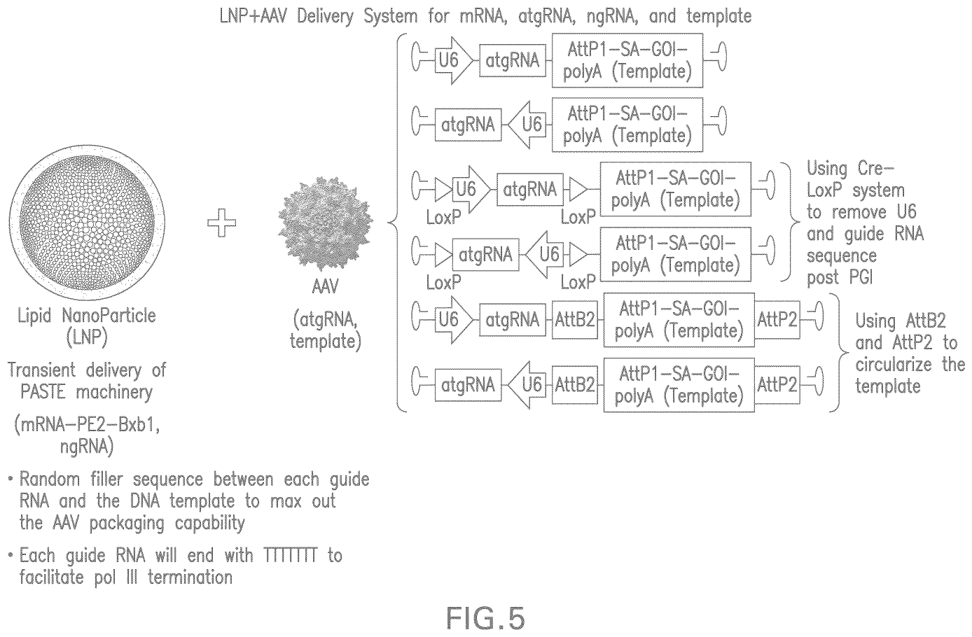

A gene editor polynucleotide construct and a nicking guide RNA (ngRNA) are packaged into a LNP (FIG. 5), wherein the gene editor polynucleotide sequence comprises a polynucleotide sequence encoding a prime editor protein linked to an integrase via peptide linker.

A polynucleotide sequence encoding an attachment site-containing guide RNA (atgRNA) and donor template are packaged in an AAV vector (FIG. 5).

Co-administration of the gene editor construct and ngRNA packaged LNP and the atgRNA, donor template packaged AAV co-delivers the gene editor construct to a cell. Integrase-mediated self-circularization of donor template occurs at integration target recognition sites within the AAV genome (FIG. 3). By use of programmable genome editing to place an orthogonal integrase landing site (i.e., distinct att site from att sites used for self-circularization) at a desired location in the genome, the direct activity of the associated integrase to the specific genomic site is guided. Gene editor construct expression, with atgRNA, ngRNA, and template co-delivery and integrase-mediated circularization of template, results in integration of template “cargo” at a precisely defined target location.

### 7.5. Example 5: Intramolecular Circularization of Plasmid and Packaged AAV Genomes

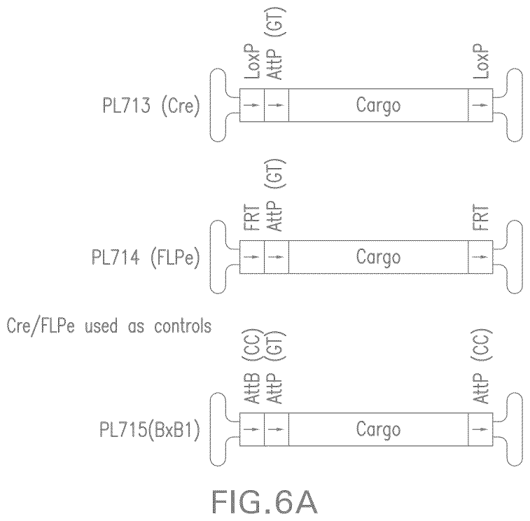

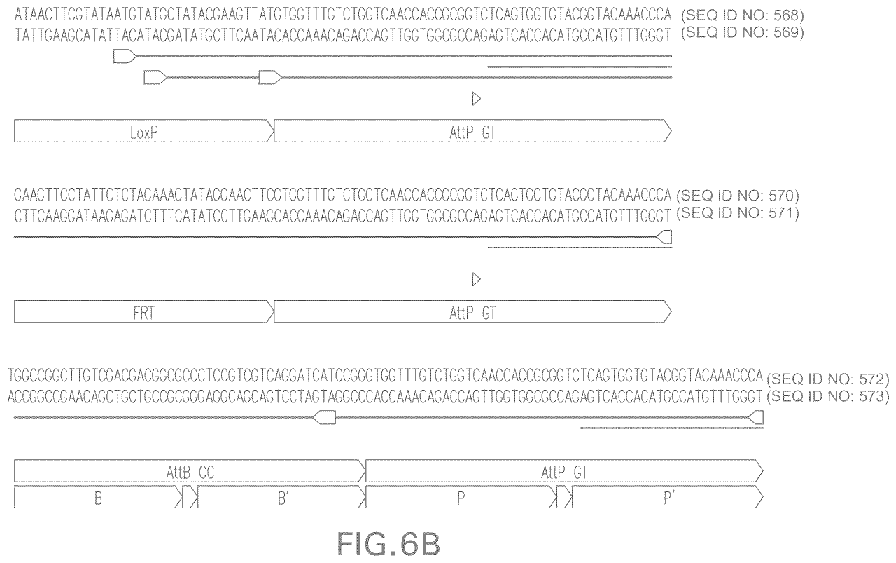

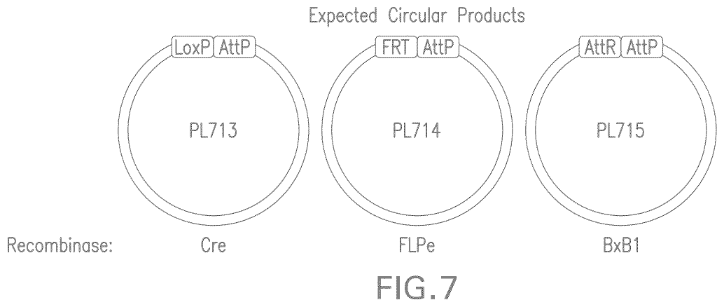

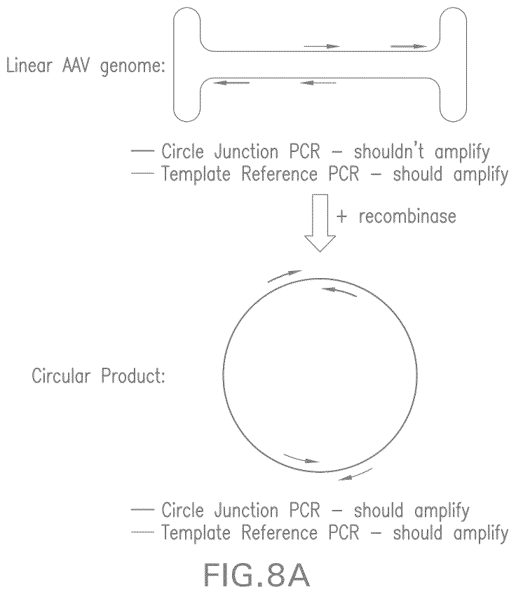

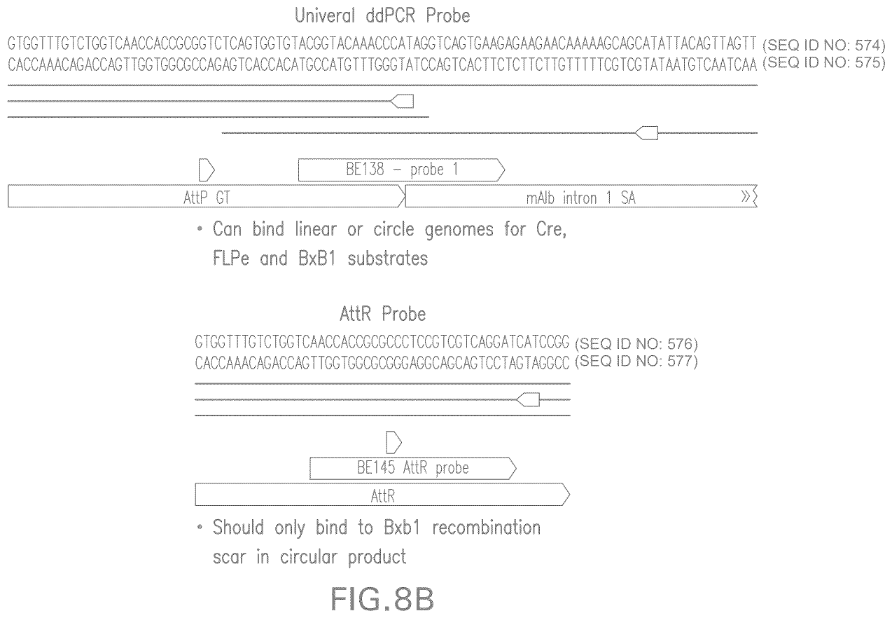

Three self-complementary AAV (scAAV) genomes were designed and generated to verify recombinase/integrase-mediated intramolecular circularization of a DNA cargo from within a linear AAV genome (FIGS. 6A-6B). Circularization of a scAAV genome is mediated by one of Cre, FLPe (thermostable mutant), or Bxb1. Further, the scAAV genomes are comprised of a DNA cargo of interest (“payload”) and an attP site (GT central dinucleotide for circularization orthogonality) for gene insertion into a genome placed attB beacon site. Expected recombinase/integrase-mediated intramolecular circularization products are illustrated in FIG. 7. A universal ddPCR probe capable of binding any linear or circularized AAV genome was designed, wherein the universal ddPCR probe is designed to only give signal upon cognate recombinase/integrase mediated circularization (FIGS. 8A-8B). Circularization products are amplified by use of a circle junction PCR primer set that is designed to amplify only circular products due to primer direction constraints. To confirm Bxb1 mediated circularization specifically, an attR scar quencher-fluorophore probe was designed. In addition, a template reference primer set was designed and generated to quantify total template DNA (linear or circular confirmation) (FIGS. 8A-8B).

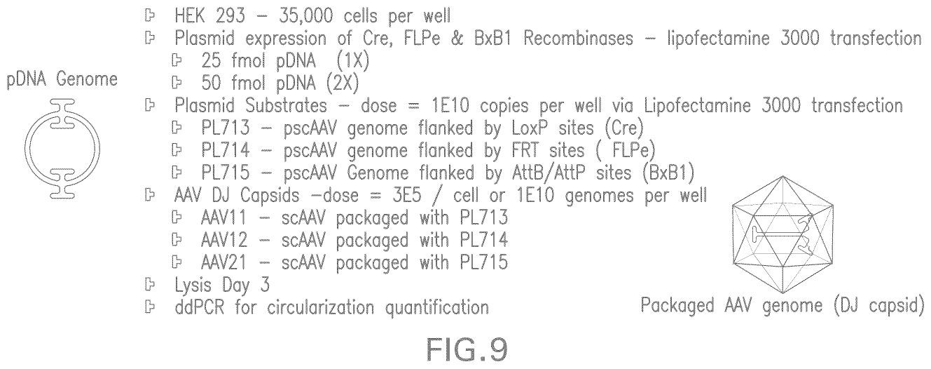

Intracellular circularization of either plasmid or packaged AAV genomes were screened in HEK293 cells (35K cells per well) (FIG. 9). Plasmids (25 fmol pDNA=1× or 50 fmol pDNA=2×) encoding one of Cre, FLPe, or Bxb1 were transfected by Lipofectamine 3000. Plasmid genome substrates were transfected at a dose of 1E10 copies per well using Lipofectamine 3000 (FIG. 9). Additionally, AAV genomes were packaged in AAV-DJ capsids and delivered at a dose of 3E5 genomes per cell or 1E10 genomes per well. Circularization ddPCR analysis was conducted three days post transfection.

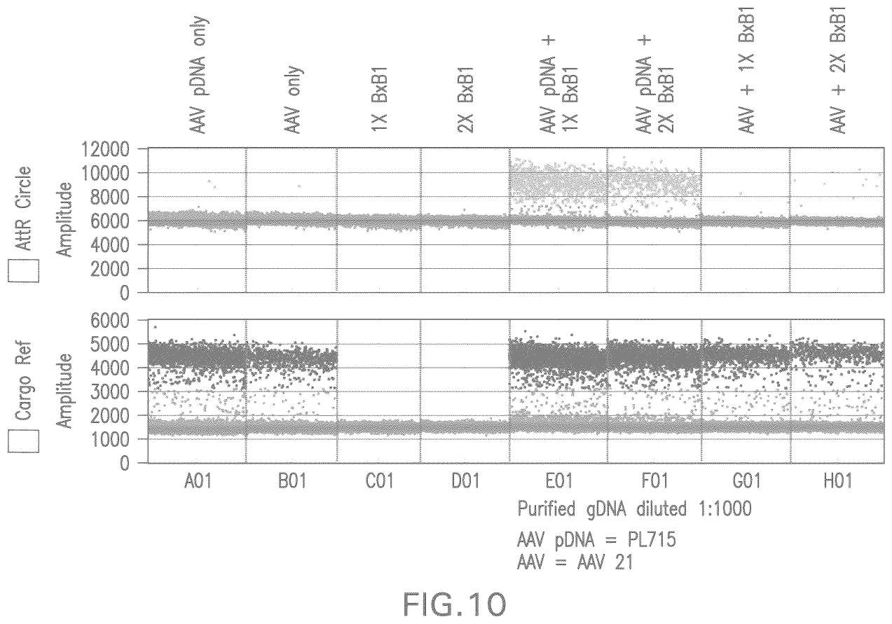

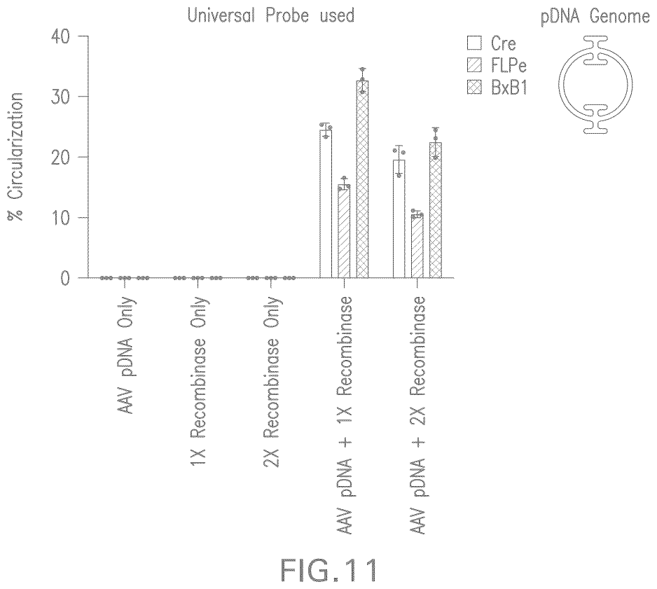

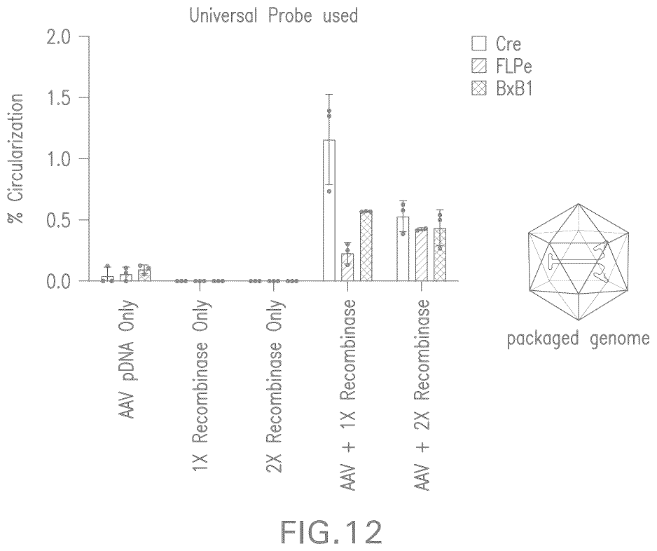

FIG. 10 demonstrates circularization of AAV pDNA and packaged AAV genomic DNA for both 1×Bxb1 and 2×Bxb1 conditions (confirmed by use of attR ddPCR primer set). Further, replicates that lacked either Bxb1 or AAV pDNA substrate demonstrated insignificant circularization. All three of the Cre-, FLPe-, and Bxb1-targeted AAV pDNA substrates demonstrated circularization upon cognate recombinase/integrase introduction, as confirmed by using the universal ddPCR probe (FIG. 11). Moreover, Cre-, FLPe-, and Bxb1-mediated circularization of packaged AAV DJ genomes substrates were demonstrated and confirmed using the universal ddPCR probe (FIG. 12).

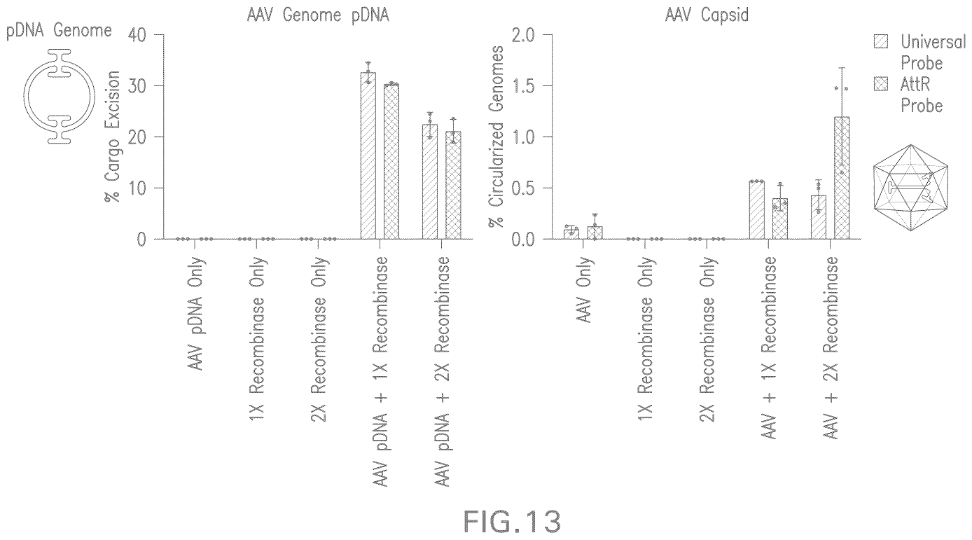

As shown in FIG. 13, the Bxb1-mediated attR scar probe provided similar percent circularization quantification compared to the universal probe.

### 7.6. Example 6: In Vitro Beacon Placement in Primary Mouse Hepatocytes and Primary Human Hepatocytes Using mRNA and AAV for Co-Delivery

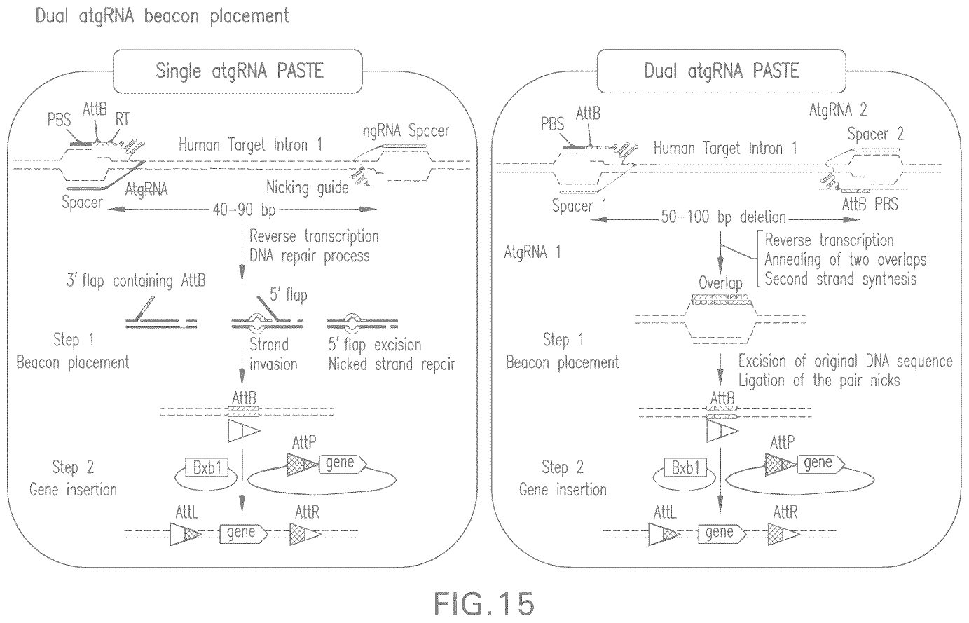

This example assessed the efficiency of in vitro beacon placement in primary human hepatocytes using mRNA delivering of a polynucleotide encoding a gene editor polynucleotide construct and AAV to deliver the first and second atgRNA. See FIG. 15 for a non-limiting example of a dual atgRNA-mediated insertion of an integration recognition site.

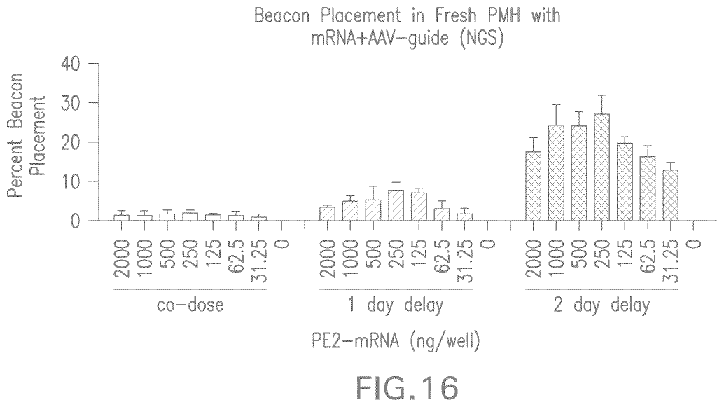

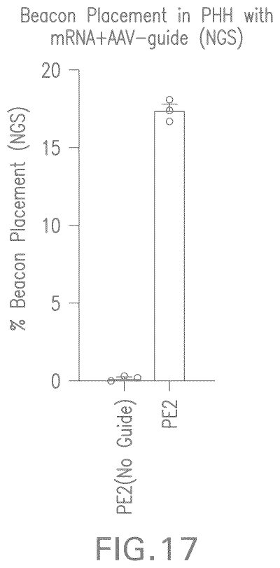

In the mouse experiments, the mRNA and AAV were delivered into the primary mouse hepatocytes (PMH) using (i) concurrent delivery (“co-dose”), (ii) AAV delivery followed by a “1-day delay” before delivery of the mRNA, or (iii) AAV delivery followed by a “2-day delay” before delivery of the mRNA. Beacon placement was then assessed using next-generation sequencing of DNA isolated from cells subjected to the delivery conditions mentioned above. The mRNA encoding the gene editor polynucleotide construct was delivered in various amounts per well: 2000 ng, 1000 ng, 500 ng, 250 ng, 125 ng, 62.5 ng, and 31.25 ng. AAV encoding the first and second atgRNA (see Table 12). The primary mouse hepatocyte data is shown in FIG. 16 and the human primary hepatocyte data is shown in FIG. 17.

TABLE 12
atgRNAs

| SEQ | | | |
|---|---|---|---|
| ID | | | |
| NO: | Target | Name | Sequence |
| 559 | Mouse | AAV-mNolc1- | GACGCGTTTTACCCGGAGCAGTTTAAGAGCTATGCTGGA |
| | Nolc1 | F | GAACACATAGCAAGTTTAAATAAGGCTAGTCCGTTATCA |
| | | (AAVG023) | ACTTGAAAAAGTGGCACCGAGTCGGTGCACGACGGAGAC |
| | | | CGCCGTCGTCGACAAGCCTCCGGGTAAAACG |
| 560 | Mouse | AAV-mNolc1- | ACAAGGGGATAAAGGTCGCTGTTTAAGAGCTATGCTGGA |
| | Nolc1 | R | AACAGCATAGCAAGTTTAAATAAGGCTAGTCCGTTATCA |
| | | | ACTTGAAAAAGTGGCACCGAGTCGGTGCACGACGGCGGT |
| | | | CTCCGTCGTCAGGATCATGACCTTTATCCCC |
| 561 | Human | AAV-hF9-F | CTTGTATGCCCCGAGAAGTGGTTTTAGAGCTAGAAATAG |
| | Factor IX | (AAVG048) | CAAGTTAAAATAAGGCTAGTCCGTTATCAACTTGAAAAA |
| | | | GTGGCACCGAGTCGGTGCACGACGGAGACCGCCGTCGTC |
| | | | GACAAGCCTTCTCGGGGCATA |
| 562 | Human | AAV-hF9-R | TATATATACTTGCTAGGGCTGTTTTAGAGCTAGAAATAG |
| | Factor IX | (AAVG048) | CAAGTTAAAATAAGGCTAGTCCGTTATCAACTTGAAAAA |
| | | | GTGGCACCGAGTCGGTGCACGACGGCGGTCTCCGTCGTC |
| | | | AGGATCATCCTAGCAAGTATA |

As shown in FIG. 16, in primary mouse hepatocytes (PMH) delivering the first atgRNAs (SEQ ID NO: 543) and the second atgRNA (SEQ ID NO: 544) using AAV at day 0 and then delivering the mRNA encoding the gene editing polynucleotide construct at day 2 (“2 day delay”) resulted in greater than 10% beacon placement for each amount of mRNA tested. Surprisingly, a 2 day delay resulted in greater beacon placement than either no delay (“co-dose) or a 1 day delay.

As shown in FIG. 17, in primary human hepatocytes (PHH), using AAV to deliver the first atgRNA (SEQ ID NO: 545) and the second atgRNA (SEQ ID NO: 546) and mRNA to deliver the gene editing polynucleotide construct resulted in about 17% beacon placement.

Taken together, this data showed robust ex vivo beacon placement in primary mouse and primary human hepatocytes.

### 7.7. Example 7: In Vivo Beacon Placement with mRNA+AAV Guide

In vivo beacon placement in mice was assessed using AAV to deliver the first and second atgRNAs and mRNA to delivery the gene editing polynucleotide construct.

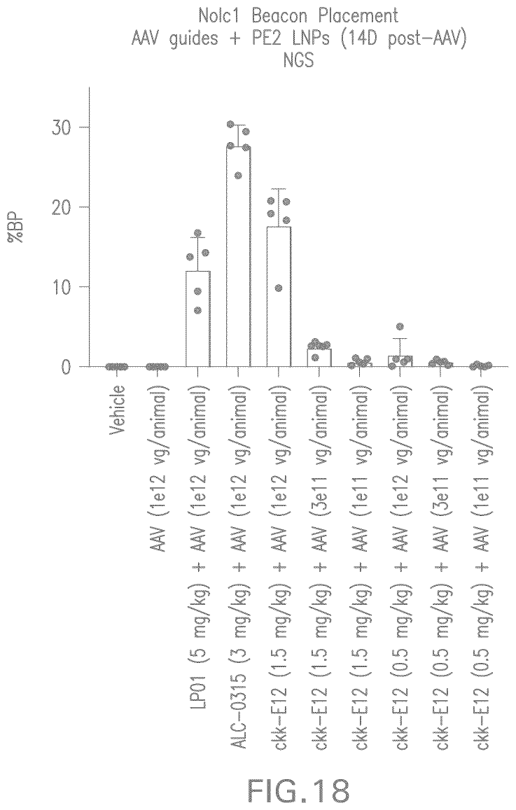

In these experiments, mice were administered AAV containing the first atgRNA (SEQ ID NO: 543; Table 12) and the second atgRNA (SEQ ID NO: 544) targeting the Nolc1 locus at 3E11 to 1E12 vector genomes (vg) per animal two 2 weeks prior to administration of the mRNA containing the gene editing polynucleotide construct (see FIG. 18). mRNA was delivered using various LNP formulations (e.g., LNP #F1, LNP #F2, and LNP #F3) at concentrations ranging from 5 mg/kg to 0.5 mg/kg via intravenous injection (see FIG. 18). After delivery of the mRNA, liver tissue was harvested, genomic DNA was isolated, and beacon efficiency was assessed by NGS. As shown in FIG. 18, three conditions resulted in vivo beacon placement efficiency greater than 10%.

Taken together, this data provided proof-of-concept for successful in vivo beacon placement using AAV to deliver the first and second atgRNA and LNPs to deliver the mRNA encoding the gene editor polynucleotide construct.

### 7.8. Example 8: In Vivo Integration in Mice Using AAV to Deliver the Template Polynucleotide and Adenovirus to Deliver BxB1

In vivo integration efficiency in AttP mice was assessed using adenovirus to deliver an integrase (e.g., Bxb1) and an AAV to deliver the template polynucleotide.

For these experiments, the adenovirus (i.e., adenovirus containing polynucleotide encoding the integrase) and the AAV (i.e., AAV containing the template polynucleotide and an attB site) were administered to mice containing dual AttP sites integrated in to the Rosa26 locus (B6.RosaBxb-GT/GA; female, Strain #036152). The Rosa26 locus included a first AttP site comprising a GT dinucleotide and a second AttP site comprising a GA dinucleotide. The AAV was a scAAV8 containing a vector having a template polynucleotide and a 38 bp GT AttB site. The Adenovirus was an adenovirus-type 5 (Ad5) containing a polynucleotide encoding Bxb1 (“Bxb1 AdV”) (SEQ ID NO: 563; Table 14). Mice were administered the adenovirus and AAV according to the experimental details in Table 13.

TABLE 13
Experimental Details for assessment of in vivo integration efficiency

| | | Bxb1 AdV dose | Cargo AAV | | Volume | Conc. | Time |
|---|---|---|---|---|---|---|---|
| Group | n | (vg/animal) | Dose (vg/animal) | Route | (ul) | (vg/ml) | points |
| 1 | 1F, 2M | vehicle | | IV | 100 | | Liver |
| 2 | 5 | 3E10 | 1E12 | IV | 100 | 3E11+1E13 | punches |
| 3 | 5 | 1E11 | 1E12 | IV | 100 | 1E12+1E13 | at 10 |
| | | | | | | | days post- |
| | | | | | | | dose |

TABLE 14
Adenovirus Vector

| Vectors | Sequence |
|---|---|
| Bxb1 AdV | TCGCGCGTTTCGGTGATGACGGTGAAAACCTC |
| (SEQ ID | TGACACATGCAGCTCCCGGAGACGGTCACAGC |
| NO: 563) | TTGTCTGTAAGCGGATGCCGGGAGCAGACAAG |
| | CCCGTCAGGGCGCGTCAGCGGGTGTTGGCGGG |
| | TGTCGGGGCTGGCTTAACTATGCGGCATCAGA |
| | GCAGATTGTACTGAGAGTGCACCATATGCGGT |
| | GTGAAATACCGCACAGATGCGTAAGGAGAAAA |
| | TACCGCATCAGGCGCCATTCGCCATTCAGGCT |
| | GCGCAACTGTTGGGAAGGGCGATCGGTGCGGG |
| | CCTCTTCGCTATTACGCCAGCTGGCGAAAGGG |
| | GGATGTGCTGCAAGGCGATTAAGTTGGGTAAC |
| | GCCAGGGTTTTCCCAGTCACGACGTTGTAAAA |
| | CGACGGCCAGTGAATTCGAGCTCTCGCTATTA |
| | CTTGGCCACTCCCTCTCTGCGCGCTCGCTCGC |
| | TCACTGAGGCCGGGCGACCAAAGGTCGCCCGA |
| | CGCCCGGGCTTTGCCCGGGCGGCCTCAGTGAG |
| | CGAGCGAGCGCGCAGAGAGGGAGTGGCCAACT |
| | CCATCACTAGGGGTTCCTCACTGCCCGCAGAT |
| | CTACTAGTGGCTTGTCGACGACGGCGGTCTCC |
| | GTCGTCAGGATCATTAGGTCAGTGAAGAGAAG |
| | AACAAAAAGCAGCATATTACAGTTAGTTGTCT |
| | TCATCAATCTTTAAATATGTTGTGTGGTTTTT |
| | CTCTCCCTGTTTCCACAGTTATGGGCAACAGC |
| | TTCAGCACCAGCGCCTTCGGCCCTGTGGCCTT |
| | TTCTCTGGGCCTCCTGCTCGTGCTGCCTGCCG |
| | CTTTTCCAGCTCCTGTGTTCACCCTGGAAGAT |
| | TTCGTGGGAGATTGGCGGCAGACCGCCGGCTA |
| | CAACCTGGACCAAGTGCTGGAACAGGGCGGAG |
| | TGTCCAGCCTGTTTCAGAACCTGGGCGTCTCC |
| | GTGACCCCTATCCAGCGGATCGTGCTGAGCGG |
| | CGAGAACGGCCTGAAAATCGACATCCATGTGA |
| | TTATCCCCTACGAGGGCCTGAGCGGAGATCAG |
| | ATGGGCCAGATCGAGAAAATCTTCAAGGTGGT |
| | GTACCCCGTCGACGACCACCACTTCAAGGTGA |
| | TCCTGCACTACGGCACCCTGGTGATCGACGGC |
| | GTTACCCCTAACATGATCGACTACTTCGGCAG |
| | ACCCTATGAGGGAATTGCCGTGTTCGACGGCA |
| | AGAAAATCACCGTGACCGGCACACTGTGGAAC |
| | GGCAACAAGATCATCGATGAGCGCCTGATCAA |
| | CCCAGACGGCAGCCTGCTGTTCAGAGTGACAA |
| | TCAATGGCGTGACAGGCTGGAGACTTTGTGAA |
| | AGAATCCTGGCCGGTTCTGGCGAGGGCAGAGG |
| | ATCTCTGCTGACATGCGGCGATGTGGAAGAGA |
| | ATCCTGGACCTGCTATGAAAATCGAGTGCAGA |
| | ATTACAGGCACACTGAACGGAGTTGAATTCGA |
| | GCTGGTCGGCGGAGGCGAGGGCACACCTGAGC |
| | AGGGCAGAATGACCAACAAGATGAAAAGCACC |
| | AAGGGCGCCCTGACCTTTTCTCCTTACCTGCT |
| | GAGCCACGTGATGGGCTATGGCTTCTACCACT |
| | TCGGCACCTACCCCAGCGGCTATGAAAACCCC |
| | TTCCTGCATGCTATCAACAACGGAGGCTACAC |
| | CAATACCAGAATCGAGAAGTACGAGGACGGCG |
| | GCGTGCTGCACGTGTCCTTCAGCTACAGATAC |
| | GAGGCCGGCAGAGTGATCGGCGACTTCAAGGT |
| | GGTGGGCACAGGATTTCCAGAAGATAGCGTGA |
| | TCTTCACCGACAAGATCATCCGGAGCAACGCC |
| | ACCGTGGAACACCTGCACCCCATGGGCGATAA |
| | TGTGCTGGTGGGCTCCTTTGCTAGAACATTCT |
| | CCCTGCGGGACGGCGGATACTACAGCTTCGTG |
| | GTCGACAGCCACATGCACTTCAAGTCTGCCAT |
| | CCACCCTTCTATCCTGCAGAACGGCGGACCTA |
| | TGTTCGCCTTCCGGCGGGTGGAGGAACTCCAC |
| | AGCAACACCGAGCTGGGCATCGTGGAATACCA |
| | GCACGCCTTTAAGACCCCTATCGCCTTCGCCA |
| | GAAGCAGAGCCAGGTGAGAGTTTAAACCCGCT |
| | GATCAGCCTCGACTGTGCCTTCTAGTTGCCAG |
| | CCATCTGTTGTTTGCCCCTCCCCCGTGCCTTC |
| | CTTGACCCTGGAAGGTGCCACTCCCACTGTCC |
| | TTTCCTAATAAAATGAGAAAATTGCATCGCAT |
| | TGTCTGAGTAGGTGTCATTCTATTCTGGGGGG |
| | TGGGGTGGGGCAGGACAGCAAGGGGGAGGATT |
| | GGGAAGACAATAGCAGGCATGCTGGGGATGCG |
| | GTGGGCTCTATGGACTAGTAGATCTCACTGCC |
| | CGCCCACTCCCTCTCTGCGCGCTCGCTCGCTC |
| | ACTGAGGCCGGGCGACCAAAGGTCGCCCGACG |
| | CCCGGGCTTTGCCCGGGCGGCCTCAGTGAGCG |
| | AGCGAGCGCGCAGAGAGGGATGCATTAATGGG |
| | ATCCTCTAGAGTCGACCTGCAGGCATGCAAGC |
| | TTGGCGTAATCATGGTCATAGCTGTTTCCTGT |
| | GTGAAATTGTTATCCGCTCACAATTCCACACA |
| | ACATACGAGCCGGAAGCATAAAGTGTAAAGCC |
| | TGGGGTGCCTAATGAGTGAGCTAACTCACATT |
| | AATTGCGTTGCGCTCACTGCCCGCTTTCCAGT |
| | CGGGAAACCTGTCGTGCCAGCTGCATTAATGA |
| | ATCGGCCAACGCGCGGGGAGAGGCGGTTTGCG |
| | TATTGGGCGCTCTTCCGCTTCCTCGCTCACTG |
| | ACTCGCTGCGCTCGGTCGTTCGGCTGCGGCGA |
| | GCGGTATCAGCTCACTCAAAGGCGGTAATACG |
| | GTTATCCACAGAATCAGGGGATAACGCAGGAA |
| | AGAACATGTGAGCAAAAGGCCAGCAAAAGGCC |
| | AGGAACCGTAAAAAGGCCGCGTTGCTGGCGTT |
| | TTTCCATAGGCTCCGCCCCCCTGACGAGCATC |
| | ACAAAAATCGACGCTCAAGTCAGAGGTGGCGA |
| | AACCCGACAGGACTATAAAGATACCAGGCGTT |
| | TCCCCCTGGAAGCTCCCTCGTGCGCTCTCCTG |
| | TTCCGACCCTGCCGCTTACCGGATACCTGTCC |
| | GCCTTTCTCCCTTCGGGAAGCGTGGCGCTTTC |
| | TCATAGCTCACGCTGTAGGTATCTCAGTTCGG |
| | TGTAGGTCGTTCGCTCCAAGCTGGGCTGTGTG |
| | CACGAACCCCCCGTTCAGCCCGACCGCTGCGC |
| | CTTATCCGGTAACTATCGTCTTGAGTCCAACC |
| | CGGTAAGACACGACTTATCGCCACTGGCAGCA |
| | GCCACTGGTAACAGGATTAGCAGAGCGAGGTA |
| | TGTAGGCGGTGCTACAGAGTTCTTGAAGTGGT |
| | GGCCTAACTACGGCTACACTAGAAGGACAGTA |
| | TTTGGTATCTGCGCTCTGCTGAAGCCAGTTAC |
| | CTTCGGAAAAAGAGTTGGTAGCTCTTGATCCG |
| | GCAAACAAACCACCGCTGGTAGCGGTGGTTTT |
| | TTTGTTTGCAAGCAGCAGATTACGCGCAGAAA |
| | AAAAGGATCTCAAGAAGATCCTTTGATCTTTT |
| | CTACGGGGTCTGACGCTCAGTGGAACGAAAAC |
| | TCACGTTAAGGGATTTTGGTCATGAGATTATC |
| | AAAAAGGATCTTCACCTAGATCCTTTTAAATT |
| | AAAAATGAAGTTTTAAATCAATCTAAAGTATA |
| | TATGAGTAAACTTGGTCTGACAGTTACCAATG |
| | CTTAATCAGTGAGGCACCTATCTCAGCGATCT |
| | GTCTATTTCGTTCATCCATAGTTGCCTGACTC |
| | CCCGTCGTGTAGATAACTACGATACGGGAGGG |
| | CTTACCATCTGGCCCCAGTGCTGCAATGATAC |
| | CGCGAGACCCACGCTCACCGGCTCCAGATTTA |
| | TCAGCAATAAACCAGCCAGCCGGAAGGGCCGA |
| | GCGCAGAAGTGGTCCTGCAACTTTATCCGCCT |
| | CCATCCAGTCTATTAATTGTTGCCGGGAAGCT |
| | AGAGTAAGTAGTTCGCCAGTTAATAGTTTGCG |
| | CAACGTTGTTGCCATTGCTACAGGCATCGTGG |
| | TGTCACGCTCGTCGTTTGGTATGGCTTCATTC |
| | AGCTCCGGTTCCCAACGATCAAGGCGAGTTAC |
| | ATGATCCCCCATGTTGTGCAAAAAAGCGGTTA |
| | GCTCCTTCGGTCCTCCGATCGTTGTCAGAAGT |
| | AAGTTGGCCGCAGTGTTATCACTCATGGTTAT |
| | GGCAGCACTGCATAATTCTCTTACTGTCATGC |
| | CATCCGTAAGATGCTTTTCTGTGACTGGTGAG |
| | TACTCAACCAAGTCATTCTGAGAATAGTGTAT |
| | GCGGCGACCGAGTTGCTCTTGCCCGGCGTCAA |
| | TACGGGATAATACCGCGCCACATAGCAGAACT |
| | TTAAAAGTGCTCATCATTGGAAAACGTTCTTC |
| | GGGGCGAAAACTCTCAAGGATCTTACCGCTGT |
| | TGAGATCCAGTTCGATGTAACCCACTCGTGCA |
| | CCCAACTGATCTTCAGCATCTTTTACTTTCAC |
| | CAGCGTTTCTGGGTGAGCAAAAACAGGAAGGC |
| | AAAATGCCGCAAAAAAGGGAATAAGGGCGACA |
| | CGGAAATGTTGAATACTCATACTCTTCCTTTT |
| | TCAATATTATTGAAGCATTTATCAGGGTTATT |
| | GTCTCATGAGCGGATACATATTTGAATGTATT |
| | TAGAAAAATAAACAAATAGGGGTTCCGCGCAC |
| | ATTTCCCCGAAAAGTGCCACCTGACGTCTAAG |
| | AAACCATTATTATCATGACATTAACCTATAAA |
| | AATAGGCGTATCACGAGGCCCTTTCGTC |

Ten days after administration of the AdV and AAV viruses, liver punches were collected and genomic DNA was isolated. ddPCR of the genomic DNA was used to assess integration efficiency.

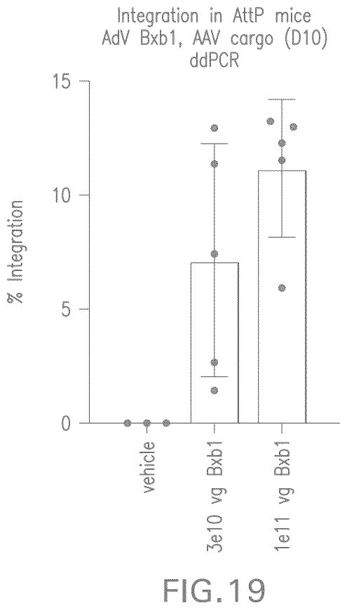

As shown in FIG. 19, administering the AAV and AdV resulted in in vivo integration of the donor polynucleotide template into the AttP mice. In particular, 3E10 vg/animal BxB1 AdV resulted in about 7% in vivo integration efficiency (see FIG. 19). Administering increased amounts of BxB1 AdV, 1E11 vg/animal, resulted in higher integration efficiency, about 11%, in AttP mice than with lower amount of 3E10 vg/animal (see FIG. 19).

Overall, this data establishes proof-of-concept for in vivo integration using an adenovirus to deliver and drive expression of Bxb1 and an AAV to deliver the template polynucleotide to be integrated into a mammalian genome, in this case, the mouse genome.

### 7.9. Example 9: In Vivo Beacon Placement in Neonatal Mice Using Split LNP

In vivo beacon placement was assessed in neonatal mice following administration of a single dose of a mixture of two LNPs. The first LNP contained mRNA encoding a prime editing system and a first synthetic atgRNA (atgRNA1). The mRNA and atgRNA1 were included at 1:1 ratio in the first LNP. The second LNP contained mRNA encoding a prime editing system and a second synthetic atgRNA (atgRNA2). The mRNA and atgRNA2 were included at a 1:1 ratio in the second LNP. Each of the first and second atgRNAs targeted the mouse Nolc1 locus and each encoded a portion of an integration recognition site (a “beacon”). AtgRNA1 and atgRNA2 together included a 6 bp overlap. The first and second LNPs were combined 1:1 as mixture prior to administration. The first atgRNA and second atgRNA are provide in Table 15, where the atgRNA include one or more 2′O-methyl modifications and one or more phosphorothioate linkages.

TABLE 15
atgRNAs

| SEQ | | | |
|---|---|---|---|
| ID | | | |
| NO: | Target | Name | Sequence |
| 564 | Mouse | mNolc1-F | mG*mA*mC*rGrCrGrUrUrUrUrArCrCrCrGrGrA |
| | Nolc1 | (synthetic | rGrCrArGrUrUrUrUrArGrAmGmCmUmAmGmAmAm |
| | | guide, 6 bp | AmUmAmGmCrArArGrUrUrArArArArUrArArGrG |
| | | overlap) | rCrUrArGrUrCrCrGrUrUrArUrCrAmAmCmUmUm |
| | | | GmAmAmAmAmAmGmUmGmGmCmAmCmCmGmAmGmUmC |
| | | | mGmGmUmGmCrArGrArCrCrGrCrCrGrUrCrGrUr |
| | | | CrGrArCrArArGrCrCrUrCrCrGrGrGrUrArArA |
| | | | *mA*mC*mG |
| 565 | Mouse | mNolc1-R | mA*mC*mA*rArGrGrGrGrArUrArArArGrGrUrC |
| | Nolc1 | (synthetic | rGrCrUrGrUrUrUrUrArGrAmGmCmUmAmGmAmAm |
| | | guide, 6 bp | AmUmAmGmCrArArGrUrUrArArArArUrArArGrG |
| | | overlap) | rCrUrArGrUrCrCrGrUrUrArUrCrAmAmCmUmUm |
| | | | GmAmAmAmAmAmGmUmGmGmCmAmCmCmGmAmGmUmC |
| | | | mGmGmUmGmCrCrGrGrUrCrUrCrCrGrUrCrGrUr |
| | | | CrArGrGrArUrCrArUrGrArCrCrUrUrUrArUrC |
| | | | *mC*mC*mC |

The LNP mixture was administered to the neonatal mice (2-5 day old CD-1 mice) according to the experimental details in Table 16.

TABLE 16
Experimental details for in vivo beacon
placement in neonatal mice.

| | | | Dose | | Volume | Conc. | |
|---|---|---|---|---|---|---|---|
| | | | (mg/ | | (ml/ | (mg/ | |
| Group | n | Treatment | kg) | Route | kg) | ml) | Time points |
| 1 | 5 | vehicle | | IV | 5 | | Whole liver |
| 2 | 3 | LNP | 1 | IV | 5 | 0.2 | on day 8 |
| 3 | 4 | LNP | 3 | IV | 5 | 0.6 | post-dose |
| | | | | | | | (168 hours) |
| 4 | 5 | vehicle | | IV | 5 | | Liver |
| 5 | 5 | LNP | 1 | IV | 5 | 0.2 | punches |
| 6 | 5 | LNP | 3 | IV | 5 | 0.6 | (one 8 mm |
| | | | | | | | punch from |
| | | | | | | | each lobe) |
| | | | | | | | at 6 weeks |
| | | | | | | | post-dose |

Fight days after administration of the LNP mixture in vivo beacon placement was assessed. In particular, at day 8 post administration, liver samples (either whole liver for groups 1-3 or liver punches from each lobe for groups 4-6 (see Table 13)) were collected and genomic DNA was isolated. Beacon placement was detected using ddPCR and NGS.

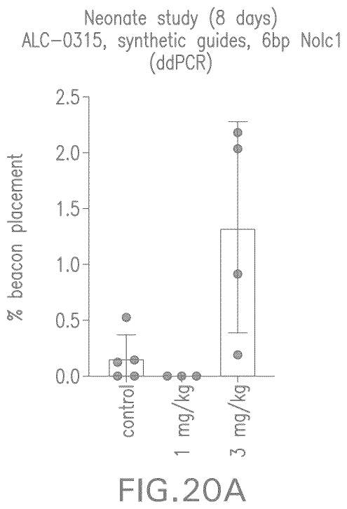

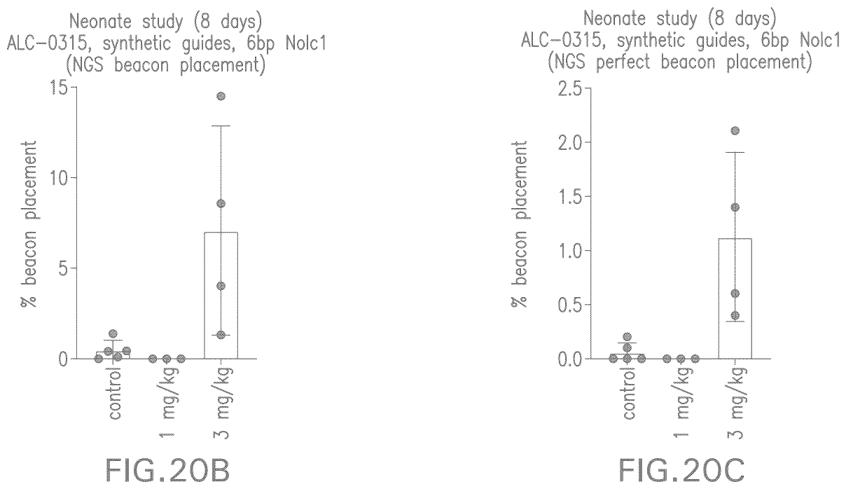

As shown in FIG. 20A, ddPCR revealed about 100 beacon placement (in Nolc1 alleles) following administration of a 3 mg/kg dose of the LNP mixture. Confirmation of beacon placement using NGS showed about 700 beacon placement (in Nolc1 alleles) following administration of a 3 mg/kg dose of the LNP mixture (see FIG. 20B). In order to determine what percentage of the integrated beacons included the expected integration recognition site (“perfect beacon”), an NGS-based assay was used to make this assessment. As shown in FIG. 20C, about 1% of the integrated beacons contained the expected integration recognition site.

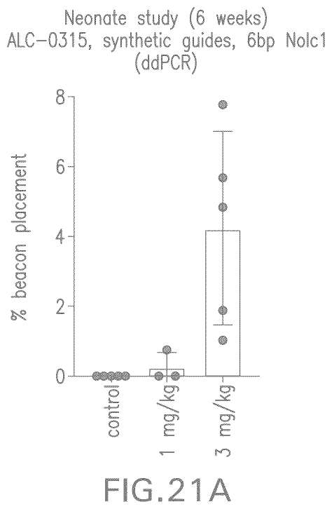

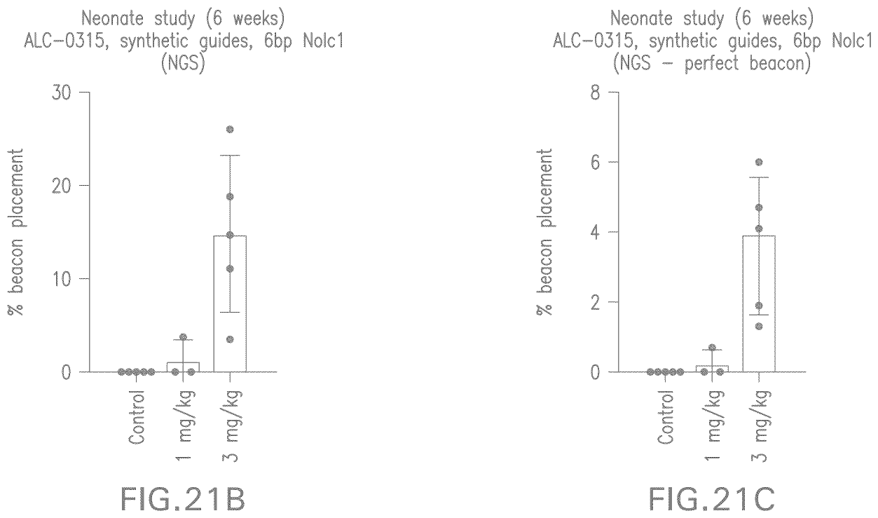

Neonates were also assessed at six weeks after administration of the LNP mixture. Beacon placement was detected using ddPCR and NGS. As shown in FIG. 21A., at six weeks post administration, ddPCR revealed about 4% beacon placement (in Nolc1 alleles) for a 3 mg/kg dose of the LNP mixture. Confirmation of beacon placement using NGS showed about 15% beacon placement (in Nolc1 alleles) for a 3 mg/kg dose of the LNP mixture (see FIG. 21B). Assessment of the percent of integrated beacons that included the expected integration recognition site (“perfect beacon”) revealed that about 3.5% of beacons were comprised of perfect beacons (see FIG. 21C).

Overall, this data demonstrated successful in vivo site-specific integration of an integration recognition site. In particular, this data showed that a split LNP approach can be used for site-specifically integrating an integration recognition site in vivo in a mammalian genome, in this case neonatal mice.

### 7.10. Example 10: In Vivo Beacon Placement in Mice Using Split LNP

In vivo beacon placement was assessed in adult mice using a single dose mixture of two LNPs. The first LNP contained mRNA encoding a prime editing system and a first synthetic atgRNA (atgRNA1). The mRNA and atgRNA1 were included at different ratios (e.g., 1:0.5, 1:1, and 1:2) ratio in the first LNP. The second LNP contained mRNA encoding a prime editing system and a second synthetic atgRNA (atgRNA2). The mRNA and atgRNA2 were included at different ratios (e.g., 1:0.5, 1:1, and 1:2) ratio in the second LNP. Here, the first and second atgRNAs targeted mouse Factor IX (“mF9”) locus and each encoded a portion of an integration recognition site (“beacon”). Similar to Example 9, atgRNA1 and atgRNA2 together included a 6 bp overlap and were combined 1:1 as mixture prior to administration. The first atgRNA and second atgRNA are provide in Table 17, where the atgRNA include one or more 2′O-methyl modifications and one or more phosphorothioate linkages.

TABLE 17
atgRNAs

| SEQ | | | |
|---|---|---|---|
| ID | | | |
| NO: | Target | Name | Sequence |
| 566 | Mouse | mF9-F | mA*mG*mU*rGrArCrArGrUrGrCrCrArGrGrA |
| | Factor IX | (synthetic guide, | rUrCrArGrGrUrUrUrUrArGrAmGmCmUmAmGm |
| | | 6 bp overlap) | AmAmAmUmAmGmCrArArGrUrUrArArArArUrA |
| | | | rArGrGrCrUrArGrUrCrCrGrUrUrArUrCrAm |
| | | | AmCmUmUmGmAmAmAmAmAmGmUmGmGmCmAmCmC |
| | | | mGmAmGmUmCmGmGmUmGmCrArGrArCrCrGrCr |
| | | | CrGrUrCrGrUrCrGrArCrArArGrCrCrArUrC |
| | | | rCrUrGrGrCrArCmU*mG*mU |
| 567 | Mouse | mF9-R | mG*mU*mU*rGrArCrArUrCrArUrGrUrCrUrG |
| | Factor IX | (synthetic guide, | rGrArGrUrGrUrUrUrUrArGrAmGmCmUmAmGm |
| | | 6 bp overlap) | AmAmAmUmAmGmCrArArGrUrUrArArArArUrA |
| | | | rArGrGrCrUrArGrUrCrCrGrUrUrArUrCrAm |
| | | | AmCmUmUmGmAmAmAmAmAmGmUmGmGmCmAmCmC |
| | | | mGmAmGmUmCmGmGmUmGmCrCrGrGrUrCrUrCr |
| | | | CrGrUrCrGrUrCrArGrGrArUrCrArUrCrCrA |
| | | | rGrArCrArUrGrAmU*mG*mU |

In particular, the LNP mixture was administered to female CD-1 mice 6-8 weeks old according to the experimental details in Table 18.

TABLE 18
Experimental details for in vivo beacon placement in adult mice

| | | Treatment | | | | | |
|---|---|---|---|---|---|---|---|
| | | (ratio | Dose | | Volume | Conc. | Time |
| Group | n | mRNA:atgRNA1:atgRNA2) | (mg/kg) | Route | (ml/kg) | (mg/ml) | points |
| 1 | 5 | vehicle | | IV | 5 | | Terminal: |
| 2 | 5 | 1:0.25:0.25* | 3 | IV | 5 | 0.6 | liver |
| 3 | 5 | 1:0.5:0.5** | 3 | IV | 5 | 0.6 | punches on |
| 4 | 5 | 1:1:1*** | 3 | IV | 5 | 0.6 | day 8 |

*1:0.25:0.25 = mRNA:atgRNA1 1:0.5; mRNA:atgRNA2 1:0.5; LNPs mixed 1:1
**1:0.5:0.5 = mRNA:atgRNA1 1:1; mRNA:atgRNA2 1:1; LNPs mixed 1:1
***1:1:1 = mRNA:atgRNA1 1:2; mRNA:atgRNA2 1:2; LNPs mixed 1:1

Eight days after administration of the LNP mixture in vivo beacon placement was assessed. In particular, at day 8 post administration, liver samples (i.e., liver punches of each lobe (see Table 14)) were collected and genomic DNA was isolated. Beacon placement was detected using ddPCR and NGS.

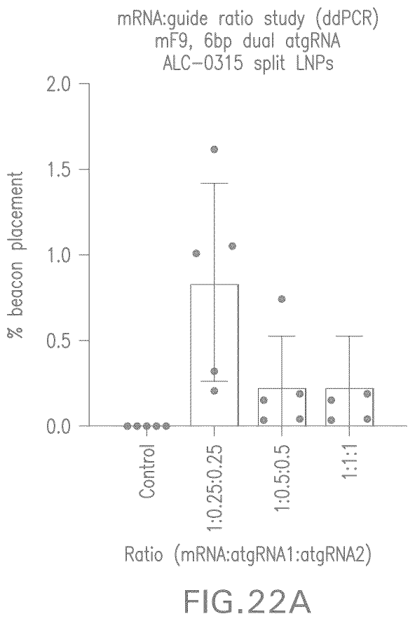

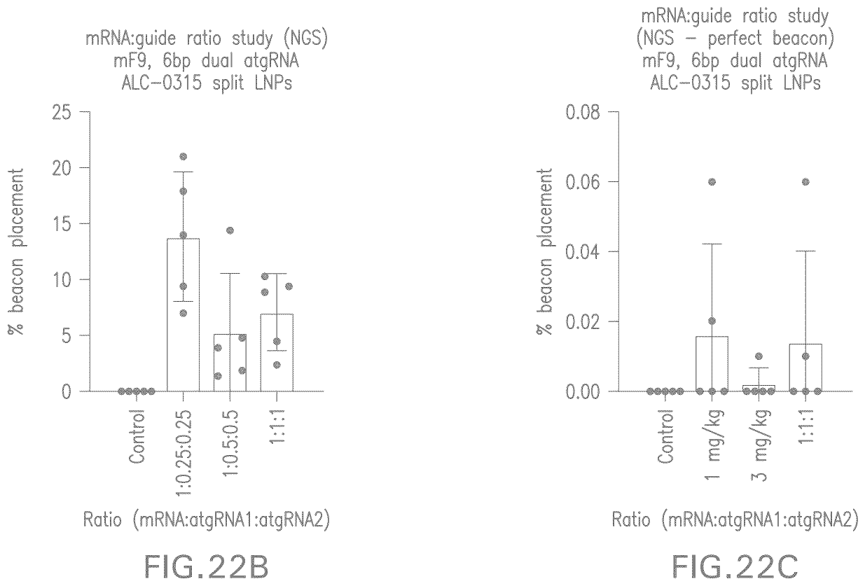

As shown in FIG. 22A, ddPCR revealed about 0.8% beacon placement (in mF9 alleles) following administration of a 1:0.25:0.25 ratio of mRNA:atgRNA1:atgRNA2. Confirmation of beacon placement using NGS showed about 14% beacon placement (in mF9 alleles) following administration of the 1:0.25:0.25 ratio of mRNA:atgRNA1:atgRNA2 (see FIG. 22B). Similar to Example 9, an NGS-based assay was used to determined what percentages of the integrated beacons included the expected integration recognition site (“perfect beacon”). As shown in FIG. 22C, about 0.02% of the beacons placed in the mF9 locus were “perfect” beacons.

Overall, this data showed successful in vivo site-specific integration of an integration recognition site in adult mice. In particular, this data showed that the ratio of mRNA to atgRNA is an important consideration in determining efficacy of in vivo site-specific integration of an integration recognition site.

### 7.11. Example 11: Assessment of Engineered Integration Enzymes

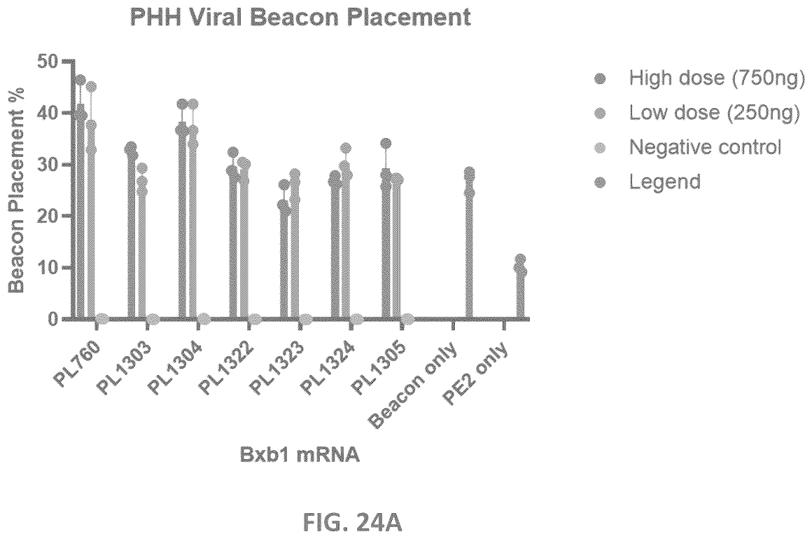

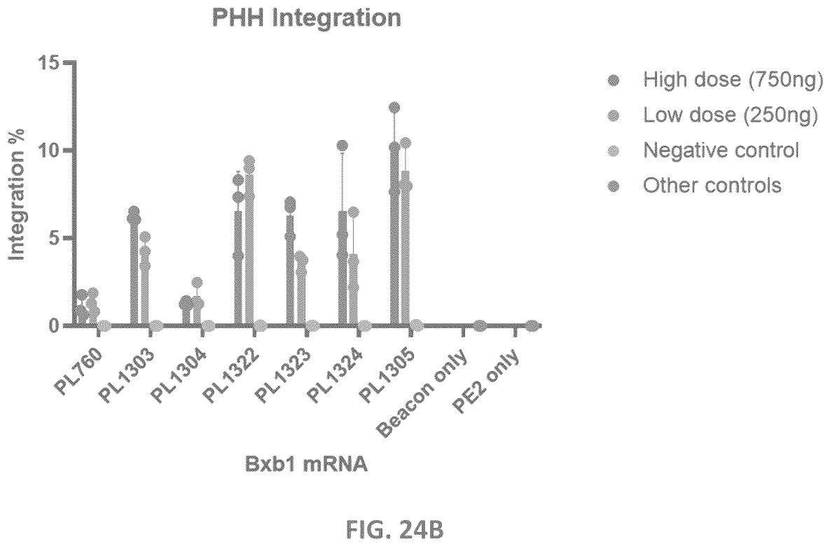

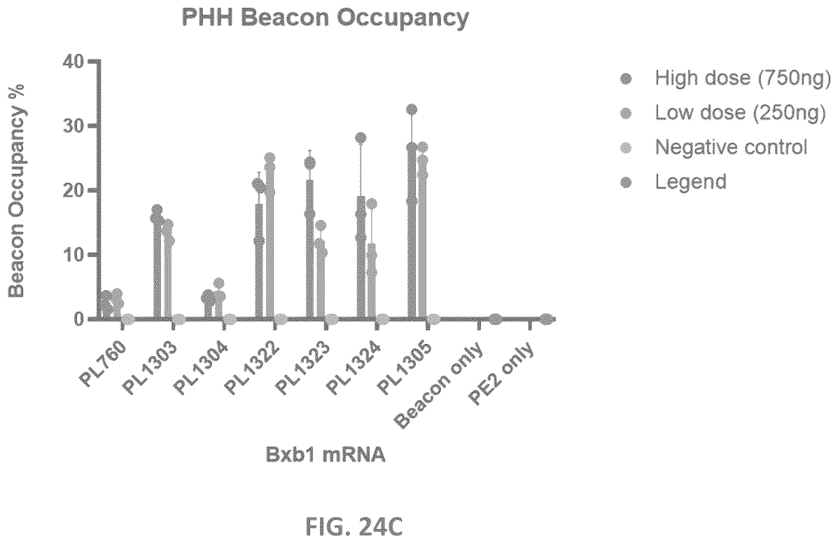

Engineered integrases described in FIG. 23 (see also Table 26) were evaluated for ability to mediate programmable gene insertion (as indicated by Integration % (see FIG. 24B), which can also be as percent “Beacon Occupancy” (see FIG. 24C). Beacon placement was used as one of the controls (see FIG. 24A).

For these experiments, the engineered integrases were introduced into a primary human hepatocyte (PHH) at a high dose (750 ng) and a low dose (250 ng) along with a gene editor polypeptide (i.e., mRNA encoding a gene editor polypeptide (i.e., a Cas9 nickase fused to a reverse transcriptase (SEQ ID NO: 589)); an AAV expressing an atgRNA (the atgRNA comprising a spacer sequence with sequence complementarity to a sequence in the human factor IX locus, a scaffold, an RT template comprising an integration recognition site, and a primer binding site (See Table 19 below); and a donor polynucleotide template comprising a cognate integration recognition site. In these experiments, the AAV used to deliver the atgRNA also included the donor polynucleotide template sequence. The AAV sequence is AAVG048 (SEQ ID NO: 592; See Table 20).

TABLE 19

| SEQ | | | |
|---|---|---|---|
| ID | | | |
| NO: | Target | Name | Sequence |
| 587 | Human | Human | TATATATACTTGCTAGGGCTgtttta |
| | Factor | Factor | gagctagaaatagcaagttaaaataa |
| | IX | IX atg | ggctagtccgttatcaacttgaaaaa |
| | | spacer | gtggcaccgagtcggtgcacgacggc |
| | | 17F | ggtctccgtcgtcaggatcatCCTAG |
| | | | CAAGTATAttttttt |
| 588 | Human | Human | CTTGTATGCCCCGAGAAGTGgtttta |
| | Factor | Factor | gagctagaaatagcaagttaaaataa |
| | IX | IX atgRNA | ggctagtccgttatcaacttgaaaaa |
| | | spacer | gtggcaccgagtcggtgcacgacgga |
| | | 17R | gaccgccgtcgtcgacaagccTTCTC |
| | | | GGGGCATAttttttt |

TABLE 20

| SEQ | | |
|---|---|---|
| ID | | |
| NO: | Name | Sequence |
| 589 | mRNA- | AGGAAGCTCAGAATAAACGCTCAACTTTGGCCGGATCTGCCACCATGAAGAGAACC |
| | PL758 | GCCGACGGCAGCGAGTTCGAGAGCCCCAAGAAGAAGAGAAAGGTGGACAAGAAG |
| | (nCas9- | TACAGCATCGGCCTGGACATCGGCACCAACAGCGTGGGCTGGGCCGTGATCACC |
| | RT | GACGAGTACAAGGTGCCCAGCAAGAAGTTCAAGGTGCTGGGCAACACCGACAGAC |
| | | ACAGCATCAAGAAGAACCTGATCGGCGCCCTGCTGTTCGACAGCGGCGAGACCGC |
| | | CGAGGCCACCAGACTGAAGAGAACCGCCAGAAGAAGATACACCAGAAGAAAGAAC |
| | | AGAATCTGCTACCTGCAGGAGATCTTCAGCAACGAGATGGCCAAGGTGGACGACA |
| | | GCTTCTTCCACAGACTGGAGGAGAGCTTCCTGGTGGAGGAGGACAAGAAGCACGA |
| | | GAGACACCCCATCTTCGGCAACATCGTGGACGAGGTGGCCTACCACGAGAAGTAC |
| | | CCCACCATCTACCACCTGAGAAAGAAGCTGGTGGACAGCACCGACAAGGCCGACC |
| | | TGAGACTGATCTACCTGGCCCTGGCCCACATGATCAAGTTCAGAGGCCACTTCCTG |
| | | ATCGAGGGCGACCTGAACCCCGACAACAGCGACGTGGACAAGCTGTTCATCCAGC |
| | | TGGTGCAGACCTACAACCAGCTGTTCGAGGAGAACCCCATCAACGCCAGCGGCGT |
| | | GGACGCCAAGGCCATCCTGAGCGCCAGACTGAGCAAGAGCAGAAGACTGGAGAA |
| | | CCTGATCGCCCAGCTGCCCGGCGAGAAGAAGAACGGCCTGTTCGGCAACCTGATC |
| | | GCCCTGAGCCTGGGCCTGACCCCCAACTTCAAGAGCAACTTCGACCTGGCCGAGG |
| | | ACGCCAAGCTGCAGCTGAGCAAGGACACCTACGACGACGACCTGGACAACCTGCT |
| | | GGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCC |
| | | CCCCTGAGCGCCAGCATGATCAAGAGATACGACGAGCACCACCAGGACCTGACCC |
| | | TGCTGAAGGCCCTGGTGAGACAGCAGCTGCCCGAGAAGTACAAGGAGATCTTCTT |
| | | CGACCAGAGCAAGAACGGCTACGCCGGCTACATCGACGGCGGCGCCAGCCAGGA |
| | | GGAGTTCTACAAGTTCATCAAGCCCATCCTGGAGAAGATGGACGGCACCGAGGAG |
| | | CTGCTGGTGAAGCTGAACAGAGAGGACCTGCTGAGAAAGCAGAGAACCTTCGACA |
| | | ACGGCAGCATCCCCCACCAGATCCACCTGGGCGAGCTGCACGCCATCCTGAGAAG |
| | | ACAGGAGGACTTCTACCCCTTCCTGAAGGACAACAGAGAGAAGATCGAGAAGATC |
| | | CTGACCTTCAGAATCCCCTACTACGTGGGCCCCCTGGCCAGAGGCAACAGCAGAT |
| | | TCGCCTGGATGACCAGAAAGAGCGAGGAGACCATCACCCCCTGGAACTTCGAGGA |
| | | GGTGGTGGACAAGGGCGCCAGCGCCCAGAGCTTCATCGAGAGAATGACCAACTTC |
| | | GACAAGAACCTGCCCAACGAGAAGGTGCTGCCCAAGCACAGCCTGCTGTACGAGT |
| | | ACTTCACCGTGTACAACGAGCTGACCAAGGTGAAGTACGTGACCGAGGGCATGAG |
| | | AAAGCCCGCCTTCCTGAGCGGCGAGCAGAAGAAGGCCATCGTGGACCTGCTGTTC |
| | | AAGACCAACAGAAAGGTGACCGTGAAGCAGCTGAAGGAGGACTACTTCAAGAAGA |
| | | TCGAGTGCTTCGACAGCGTGGAGATCAGCGGCGTGGAGGACAGATTCAACGCCAG |
| | | CCTGGGCACCTACCACGACCTGCTGAAGATCATCAAGGACAAGGACTTCCTGGAC |
| | | AACGAGGAGAACGAGGACATCCTGGAGGACATCGTGCTGACCCTGACCCTGTTCG |
| | | AGGACAGAGAGATGATCGAGGAGAGACTGAAGACCTACGCCCACCTGTTCGACGA |
| | | CAAGGTGATGAAGCAGCTGAAGAGAAGAAGATACACCGGCTGGGGCAGACTGAGC |
| | | AGAAAGCTGATCAACGGCATCAGAGACAAGCAGAGCGGCAAGACCATCCTGGACT |
| | | TCCTGAAGAGTGACGGCTTCGCCAACAGAAACTTCATGCAGCTGATCCACGACGAC |
| | | AGCCTGACCTTCAAGGAGGACATCCAGAAGGCCCAGGTGAGCGGCCAGGGCGAC |
| | | AGCCTGCACGAGCACATCGCCAACCTGGCCGGCAGCCCCGCCATCAAGAAGGGC |
| | | ATCCTGCAGACCGTGAAGGTGGTGGACGAGCTGGTGAAGGTGATGGGCAGACACA |
| | | AGCCCGAGAACATCGTGATCGAGATGGCCAGAGAGAACCAGACCACCCAGAAGGG |
| | | CCAGAAGAACAGCAGAGAGAGAATGAAGAGAATCGAGGAGGGCATCAAGGAGCTG |
| | | GGCAGCCAGATCCTGAAGGAGCACCCCGTGGAGAACACCCAGCTGCAGAACGAG |
| | | AAGCTGTACCTGTACTACCTGCAGAACGGCAGAGACATGTACGTGGACCAGGAGC |
| | | TGGACATCAACAGACTGAGCGACTACGACGTGGACGCCATCGTGCCCCAGAGCTT |
| | | CCTGAAGGACGACAGCATCGACAACAAGGTGCTGACCAGAAGCGACAAGAACAGA |
| | | GGCAAGAGCGACAACGTGCCCAGCGAGGAGGTGGTGAAGAAGATGAAGAACTACT |
| | | GGAGACAGCTGCTGAACGCCAAGCTGATCACCCAGAGAAAGTTCGACAACCTGAC |
| | | CAAGGCCGAGAGAGGCGGCCTGAGCGAGCTGGACAAGGCCGGCTTCATCAAGAG |
| | | ACAGCTGGTGGAGACCAGACAGATCACCAAGCACGTGGCCCAGATCCTGGACAGC |
| | | AGAATGAACACCAAGTACGACGAGAACGACAAGCTGATCAGAGAGGTGAAGGTGA |
| | | TCACCCTGAAGAGTAAGCTGGTGAGCGACTTCAGAAAGGACTTCCAGTTCTACAAG |
| | | GTGAGAGAGATCAACAACTACCACCACGCCCACGACGCCTACCTGAACGCCGTGG |
| | | TGGGCACCGCCCTGATCAAGAAGTACCCCAAGCTGGAGAGCGAGTTCGTGTACGG |
| | | CGACTACAAGGTGTACGACGTGAGAAAGATGATCGCCAAGAGCGAGCAGGAGATC |
| | | GGCAAGGCCACCGCCAAGTACTTCTTCTACAGCAACATCATGAACTTCTTCAAGAC |
| | | CGAGATCACCCTGGCCAACGGCGAGATCAGAAAGAGACCCCTGATCGAGACCAAC |
| | | GGCGAGACCGGCGAGATCGTGTGGGACAAGGGCAGAGACTTCGCCACCGTGAGA |
| | | AAGGTGCTGAGCATGCCCCAGGTGAACATCGTGAAGAAGACCGAGGTGCAGACCG |
| | | GCGGCTTCAGCAAGGAGAGCATCCTGCCCAAGAGAAACAGCGACAAGCTGATCGC |
| | | CAGAAAGAAGGACTGGGACCCCAAGAAGTACGGCGGCTTCGACAGCCCCACCGT |
| | | GGCCTACAGCGTGCTGGTGGTGGCCAAGGTGGAGAAGGGCAAGAGCAAGAAGCT |
| | | GAAGAGTGTGAAGGAGCTGCTGGGCATCACCATCATGGAGAGAAGCAGCTTCGAG |
| | | AAGAACCCCATCGACTTCCTGGAGGCCAAGGGCTACAAGGAGGTGAAGAAGGACC |
| | | TGATCATCAAGCTGCCCAAGTACAGCCTGTTCGAGCTGGAGAACGGCAGAAAGAG |
| | | AATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGC |
| | | CCCGAGGACAACGAGCAGAAGCAGCTGTTCGTGGAGCAGCACAAGCACTACCTGG |
| | | ACGAGATCATCGAGCAGATCAGCGAGTTCAGCAAGAGAGTGATCCTGGCCGACGC |
| | | CAACCTGGACAAGGTGCTGAGCGCCTACAACAAGCACAGAGACAAGCCCATCAGA |
| | | GAGCAGGCCGAGAACATCATCCACCTGTTCACCCTGACCAACCTGGGCGCCCCCG |
| | | CCGCCTTCAAGTACTTCGACACCACCATCGACAGAAAGAGATACACCAGCACCAAG |
| | | GAGGTGCTGGACGCCACCCTGATCCACCAGAGCATCACCGGCCTGTACGAGACCA |
| | | GAATCGACCTGAGCCAGCTGGGGGGCGACAGCGGCGGCAGCAGCGGCGGCAGC |
| | | AGCGGCAGCGAGACCCCCGGCACCAGCGAGAGCGCCACCCCCGAGAGCAGCGG |
| | | CGGCAGCAGCGGCGGCAGCAGCACCCTGAACATCGAGGACGAGTACAGACTGCA |
| | | CGAGACCAGCAAGGAGCCCGACGTGAGCCTGGGCAGCACCTGGCTGAGCGACTT |
| | | CCCCCAGGCCTGGGCCGAGACCGGCGGCATGGGCCTGGCCGTGAGACAGGCCC |
| | | CCCTGATCATCCCCCTGAAGGCCACCAGCACCCCCGTGAGCATCAAGCAGTACCC |
| | | CATGAGCCAGGAGGCCAGACTGGGCATCAAGCCCCACATCCAGAGACTGCTGGAC |
| | | CAGGGCATCCTGGTGCCCTGCCAGAGCCCCTGGAACACCCCCCTGCTGCCCGTG |
| | | AAGAAGCCCGGCACCAACGACTACAGACCCGTGCAGGACCTGAGAGAGGTGAACA |
| | | AGAGAGTGGAGGACATCCACCCCACCGTGCCCAACCCCTACAACCTGCTGAGCGG |
| | | CCTGCCCCCCAGCCACCAGTGGTACACCGTGCTGGACCTGAAGGACGCCTTCTTC |
| | | TGCCTGAGACTGCACCCCACCAGCCAGCCCCTGTTCGCCTTCGAGTGGAGAGACC |
| | | CCGAGATGGGCATCAGCGGCCAGCTGACCTGGACCAGACTGCCCCAGGGCTTCA |
| | | AGAACAGCCCCACCCTGTTCAACGAGGCCCTGCACAGAGACCTGGCCGACTTCAG |
| | | AATCCAGCACCCCGACCTGATCCTGCTGCAGTACGTGGACGACCTGCTGCTGGCC |
| | | GCCACCAGCGAGCTGGACTGCCAGCAGGGCACCAGAGCCCTGCTGCAGACCCTG |
| | | GGCAACCTGGGCTACAGAGCCAGCGCCAAGAAGGCCCAGATCTGCCAGAAGCAG |
| | | GTGAAGTACCTGGGCTACCTGCTGAAGGAGGGCCAGAGATGGCTGACCGAGGCC |
| | | AGAAAGGAGACCGTGATGGGCCAGCCCACCCCCAAGACCCCCAGACAGCTGAGA |
| | | GAGTTCCTGGGCAAGGCCGGCTTCTGCAGACTGTTCATCCCCGGCTTCGCCGAGA |
| | | TGGCCGCCCCCCTGTACCCCCTGACCAAGCCCGGCACCCTGTTCAACTGGGGCCC |
| | | CGACCAGCAGAAGGCCTACCAGGAGATCAAGCAGGCCCTGCTGACCGCCCCCGC |
| | | CCTGGGCCTGCCCGACCTGACCAAGCCCTTCGAGCTGTTCGTGGACGAGAAGCAG |
| | | GGCTACGCCAAGGGCGTGCTGACCCAGAAGCTGGGCCCCTGGAGAAGACCCGTG |
| | | GCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATG |
| | | GGCCAGCCCCTGGTGATCCTGGCCCCCCACGCCGTGGAGGCCCTGGTGAAGCAG |
| | | CCCCCCGACAGATGGCTGAGCAACGCCAGAATGACCCACTACCAGGCCCTGCTGC |
| | | TGGACACCGACAGAGTGCAGTTCGGCCCCGTGGTGGCCCTGAACCCCGCCACCC |
| | | TGCTGCCCCTGCCCGAGGAGGGCCTGCAGCACAACTGCCTGGACATCCTGGCCG |
| | | AGGCCCACGGCACCAGACCCGACCTGACCGACCAGCCCCTGCCCGACGCCGACC |
| | | ACACCTGGTACACCGACGGCAGCAGCCTGCTGCAGGAGGGCCAGAGAAAGGCCG |
| | | GCGCCGCCGTGACCACCGAGACCGAGGTGATCTGGGCCAAGGCCCTGCCCGCCG |
| | | GCACCAGCGCCCAGAGAGCCGAGCTGATCGCCCTGACCCAGGCCCTGAAGATGG |
| | | CCGAGGGCAAGAAGCTGAACGTGTACACCGACAGCAGATACGCCTTCGCCACCGC |
| | | CCACATCCACGGCGAGATCTACAGAAGAAGAGGCTGGCTGACCAGCGAGGGCAA |
| | | GGAGATCAAGAACAAGGACGAGATCCTGGCCCTGCTGAAGGCCCTGTTCCTGCCC |
| | | AAGAGACTGAGCATCATCCACTGCCCCGGCCACCAGAAGGGCCACAGCGCCGAG |
| | | GCCAGAGGCAACAGAATGGCCGACCAGGCCGCCAGAAAGGCCGCCATCACCGAG |
| | | ACCCCCGACACCAGCACCCTGCTGATCGAGAACAGCAGCCCCAGCGGCGGCAGC |
| | | AAGAGAACCGCCGACGGCAGCGAGTTCGAGCCCAAGAAGAAGAGAAAGGTGGGC |
| | | GGCAGCGTGAGCGGCTGGAGACTGTTCAAGAAGATCAGCTGATGAACCAGCCTCA |
| | | AGAACACCCGAATGGAGTCTCTAAGCTACATAATACCAACTTACACTTTACAAAATG |
| | | TTGTCCCCCAAAAATGTAGCCATTCGTATCTGCTCCTAATAAAAAGAAAGTTTCTTCA |
| | | CATTCTAAAAAAAAAAAAAAAAAAAAAAAAAAAAAAAAAAAAAAAAAAAAAAAAAAA |
| | | AAAAAAAAAAAAAAAAAAAAAAAAAAAA |
| 590 | mRNA- | AGGAAGCTCAGAATAAACGCTCAACTTTGGCCGGATCTGCCACCATGCCCAAGAAG |
| | PL1305 | AAGAGAAAGGTGGGCAGCGGCAGAGCCCTGGTGGTGATCAGACTGAGCAGAGTG |
| | (BxB1) | ACCGACGCCACCACCAGCCCCGAGAGACAGCTGGAGAGCTGCCAGCAGCTGTGC |
| | | GCCCAGAGAGGCTGGGACGTGGTGGGCGTGGCCGAGGACCTGGACGTGAGCGG |
| | | CGCCGTGGACCCCTTCGACAGAAAGAGAAGACCCAACCTGGCCAGATGGCTGGCC |
| | | TTCGAGGAGCAGCCCTTCGACGTGATCGTGGCCTACAGAGTGGACAGACTGACCA |
| | | GAAGCATCAGACACCTGCAGCAGCTGGTGCACTGGGCCGAGGACCACAAGAAGCT |
| | | GGTGGTGAGCGCCACCGAGGCCCACTTCGACACCACCACCCCCTTCGCCGCCGT |
| | | GGTGATCGCCCTGATGGGCACCGTGGCCCAGATGGAGCTGGAGGCCATCAAGGA |
| | | GAGAAACAGAAGCGCCGCCCACTTCAACATCAGAGCCGGCAAGTACAGAGGCAGC |
| | | CTGCCCCCCTGGGGCTACCTGCCCACCAGAGTGGACGGCGAGTGGAGACTGGTG |
| | | CCCGACCCCGTGCAGAGAGAGAGAATCCTGGAGGTGTACCACAGAGTGGTGGACA |
| | | ACCACGAGCCCCTGCACCTGGTGGCCCACGACCTGAACAGAAGAGGCGTGCTGA |
| | | GCCCCAAGGACTACTTCGCCCAGCTGCAGGGCAGAGAGCCCCAGGGCAGAGAGT |
| | | GGAGCGCCACCGCCCTGAAGAGAAGCATGATCAGCGAGGCCATGCTGGGCTACG |
| | | CCACCCTGAACGGCAAGACCGTGAGAGACGACGACGGCGCCCCCCTGGTGAGAG |
| | | CCGAGCCCATCCTGACCAGAGAGCAGCTGGAGGCCCTGAGAGCCGAGCTGGTGA |
| | | AGACCAGCAGAGCCAAGCCCGCCGTGAGCACCCCCAGCCTGCTGCTGAGAGTGC |
| | | TGTTCTGCGCCGTGTGCGGCGAGCCCGCCTACAAGTTCGCCGGCGGCGGCAGAA |
| | | AGCACCCCAGATACAGATGCAGAAGCATGGGCTTCCCCAAGCACTGCGGCAACGG |
| | | CACCGTGGCCATGGCCGAGTGGGACGCCTTCTGCGAGGAGCAGGTGCTGGACCT |
| | | GCTGGGCGACGCCGAGAGACTGGAGAAGGTGTGGGTGGCCGGCAGCGACAGCG |
| | | CCGTGGAGCTGGCCGAGGTGAACGCCGAGCTGGTGGACCTGACCAGCCTGATCG |
| | | GCAGCCCCGCCTACAGAGCCGGCAGCCCCCAGAGAGAGGCCCTGGACGCCAGAA |
| | | TCGCCGCCCTGGCCGCCAGACAGGAGGAGCTGGAGGGCCTGGAGGCCAGACCCA |
| | | GCGGCTGGGAGTGGAGAGAGACCGGCCAGAGATTCGGCGACTGGTGGAGAGAGC |
| | | AGGACACCGCCGCCAAGAACACCTGGCTGAGAAGCATGAACGTGAGACTGACCTT |
| | | CGACGTGAGAGGCGGCCTGACCAGAACCATCGACTTCGGCGACCTGCAGGAGTA |
| | | CGAGCAGCACCTGAGACTGGGCAGCGTGGTGGAGAGACTGCACACCGGCATGAG |
| | | CTGATGAACCAGCCTCAAGAACACCCGAATGGAGTCTCTAAGCTACATAATACCAA |
| | | CTTACACTTTACAAAATGTTGTCCCCCAAAATGTAGCCATTCGTATCTGCTCCTAATA |
| | | AAAAGAAAGTTTCTTCACATTCTAAAAAAAAAAAAAAAAAAAAAAAAAAAAAAAAAAA |
| | | AAAAAAAAAAAAAAAAAAAAAAAAAAAAAAAAAAAAAAAAAAAAA |
| 591 | mRNA- | AGGAAGCTCAGAATAAACGCTCAACTTTGGCCGGATCTGCCACCATGCCCAAGAAG |
| | PL1325 | AAGAGAAAGGTGTACCCCTACGACGTGCCCGACTACGCCAAGGACAAGAAATCCG |
| | (Bxb1 | ATGGCAAGGACAGCCAGAAAAAAGGCAGCAGAGCCCTGGTGGTGATCAGACTGAG |
| | with | CAGAGTGACCGACGCCACCACCAGCCCCGAGAGACAGCTGGAGAGCTGCCAGCA |
| | stabili- | GCTGTGCGCCCAGAGAGGCTGGGACGTGGTGGGCGTGGCCGAGGACCTGGACGT |
| | zation | GAGCGGCGCCGTGGACCCCTTCGACAGAAAGAGAAGACCCAACCTGGCCAGATG |
| | domain) | GCTGGCCTTCGAGGAGCAGCCCTTCGACGTGATCGTGGCCTACAGAGTGGACAGA |
| | | CTGACCAGAAGCATCAGACACCTGCAGCAGCTGGTGCACTGGGCCGAGGACCACA |
| | | AGAAGCTGGTGGTGAGCGCCACCGAGGCCCACTTCGACACCACCACCCCCTTCGC |
| | | CGCCGTGGTGATCGCCCTGATGGGCACCGTGGCCCAGATGGAGCTGGAGGCCAT |
| | | CAAGGAGAGAAACAGAAGCGCCGCCCACTTCAACATCAGAGCCGGCAAGTACAGA |
| | | GGCAGCCTGCCCCCCTGGGGCTACCTGCCCACCAGAGTGGACGGCGAGTGGAGA |
| | | CTGGTGCCCGACCCCGTGCAGAGAGAGAGAATCCTGGAGGTGTACCACAGAGTG |
| | | GTGGACAACCACGAGCCCCTGCACCTGGTGGCCCACGACCTGAACAGAAGAGGC |
| | | GTGCTGAGCCCCAAGGACTACTTCGCCCAGCTGCAGGGCAGAGAGCCCCAGGGC |
| | | AGAGAGTGGAGCGCCACCGCCCTGAAGAGAAGCATGATCAGCGAGGCCATGCTG |
| | | GGCTACGCCACCCTGAACGGCAAGACCGTGAGAGACGACGACGGCGCCCCCCTG |
| | | GTGAGAGCCGAGCCCATCCTGACCAGAGAGCAGCTGGAGGCCCTGAGAGCCGAG |
| | | CTGGTGAAGACCAGCAGAGCCAAGCCCGCCGTGAGCACCCCCAGCCTGCTGCTG |
| | | AGAGTGCTGTTCTGCGCCGTGTGCGGCGAGCCCGCCTACAAGTTCGCCGGCGGC |
| | | GGCAGAAAGCACCCCAGATACAGATGCAGAAGCATGGGCTTCCCCAAGCACTGCG |
| | | GCAACGGCACCGTGGCCATGGCCGAGTGGGACGCCTTCTGCGAGGAGCAGGTGC |
| | | TGGACCTGCTGGGCGACGCCGAGAGACTGGAGAAGGTGTGGGTGGCCGGCAGCG |
| | | ACAGCGCCGTGGAGCTGGCCGAGGTGAACGCCGAGCTGGTGGACCTGACCAGCC |
| | | TGATCGGCAGCCCCGCCTACAGAGCCGGCAGCCCCCAGAGAGAGGCCCTGGACG |
| | | CCAGAATCGCCGCCCTGGCCGCCAGACAGGAGGAGCTGGAGGGCCTGGAGGCCA |
| | | GACCCAGCGGCTGGGAGTGGAGAGAGACCGGCCAGAGATTCGGCGACTGGTGGA |
| | | GAGAGCAGGACACCGCCGCCAAGAACACCTGGCTGAGAAGCATGAACGTGAGACT |
| | | GACCTTCGACGTGAGAGGCGGCCTGACCAGAACCATCGACTTCGGCGACCTGCAG |
| | | GAGTACGAGCAGCACCTGAGACTGGGCAGCGTGGTGGAGAGACTGCACACCGGC |
| | | ATGAGCTGATGAACCAGCCTCAAGAACACCCGAATGGAGTCTCTAAGCTACATAAT |
| | | ACCAACTTACACTTTACAAAATGTTGTCCCCCAAAATGTAGCCATTCGTATCTGCTC |
| | | CTAATAAAAAGAAAGTTTCTTCACATTCTAAAAAAAAAAAAAAAAAAAAAAAAAAAAA |
| | | AAAAAAAAAAAAAAAAAAAAAAAAAAAAAAAAAAAAAAAAAAAAAAAAAAAt |
| 592 | AAVG0 | cgtatcacgaggccctttgtcacagcttgtctgtaagcggatgccgggagcagacaagcccgtcagggcgcgtcagcgg |
| | 48 | gtgttggcgggtgtcggggctggcttaactatgcggcatcagagcagattgtactgagagtgcaccatatgcggtgtgaaat |
| | | accgcacagatgcgtaaggagaaaataccgcatcaggcgccattcgccattcaggctgcgcaactgttgggaagggcg |
| | | atcggtgcgggcctcttcgctattacgccagctggcgaaagggggatgtgctgcaaggcgattaagttgggtaacgccag |
| | | ggttttcccagtcacgacgttgtaaaacgacggccagtgaattcGAGCTCtcgctattacTTGGCCACTCCCTC |
| | | TCTGCGCGCTCGCTCGCTCACTGAGGCCGGGCGACCAAAGGTCGCCCGACGCCC |
| | | GGGCTTTGCCCGGGCGGCCTCAGTGAGCGAGCGAGCGCGCAGAGAGGGAGTGG |
| | | CCAACTCCATCACTAGGGGTTCCTCACTGCCCGCagatctACTAGTCGTGTGGGGAC |
| | | CAGGTTTTGCCTTTAGTTTTGCACACACTGTAGTTCATCaacaaaaaaaTATACTTGCTA |
| | | GGatgatcctgacgacggagaccgccgtcgtgcaccgactcggtgccactttttcaagttgataacggactagccttatttt |
| | | aacttgctatttctagctctaaaacAGCCCTAGCAAGTATATATACggtgtttcgtcctttccacaagatatataa |
| | | agccaagaaatcgaaatactttcaagttacggtaagcatatgatagtccattttaaaacataattttaaaactgcaaactacccaa |
| | | gaaattattactttctacgtcacgtattttgtactaatatctttgtgtttacagtcaaattaattctaattatctctctaacagcctt |
| | | gtatcgtatatgcaaatatgaaggaatcatgggaaataggccctcCCTGTCTCAAAAAAAAGAAATTGGC |
| | | AGAATTAAGTAAGTTGATGTTTGCCaaaaaaaTATGCCCCGAGAAggcttgtcgacgacggcg |
| | | gtctccgtcgtgcaccgactcggtgccactttttcaagttgataacggactagccttattttaacttgctatttctagctctaaaac |
| | | CACTTCTCGGGGCATACAAGCggtgtttcgtcctttccacaagatatataaagccaagaaatcgaaatactttc |
| | | aagttacggtaagcatatgatagtccattttaaaacataattttaaaactgcaaactacccaagaaattattactttctacgtca |
| | | cgtattttgtactaatatctttgtgtttacagtcaaattaattctaattatctctctaacagccttgtatcgtatatgcaaatatgaa |
| | | ggaatcatgggaaataggccctcTTTATGGAGATGCTCATGGCCTCATTGAAGCCCCACTACAG |
| | | CTCTGGTAGCGGTAACCATGCGTATTTGACACACGAAGGAACTAGGGAAAAGGCAT |
| | | TAGGTGGTTTGTCTGGTCAACCACCGCGGTCTCAGTGGTGTACGGTACAAACCCAC |
| | | CACCATGGGCAACAGCTTCAGCACCAGCGCCTTCGGCCCTGTGGCCTTTTCTCTG |
| | | GGCCTCCTGCTCGTGCTGCCTGCCGCTTTTCCAGCTCCTGTGTTCACCCTGGAAGA |
| | | TTTCGTGGGAGATTGGCGGCAGACCGCCGGCTACAACCTGGACCAAGTGCTGGAA |
| | | CAGGGCGGAGTGTCCAGCCTGTTTCAGAACCTGGGCGTETCCGTGACCCCTATCCA |
| | | GCGGATCGTGCTGAGCGGCGAGAACGGCCTGAAAATCGACATCCATGTGATTATC |
| | | CCCTACGAGGGCCTGAGCGGAGATCAGATGGGCCAGATCGAGAAAATCTTCAAGG |
| | | TGGTGTACCCCGTCGACGACCACCACTTCAAGGTGATCCTGCACTACGGCACCCT |
| | | GGTGATCGACGGCGTTACCCCTAACATGATCGACTACTTCGGCAGACCCTATGAG |
| | | GGAATTGCCGTGTTCGACGGCAAGAAAATCACCGTGACCGGCACACTGTGGAACG |
| | | GCAACAAGATCATCGATGAGCGCCTGATCAACCCAGACGGCAGCCTGCTGTTCAG |
| | | AGTGACAATCAATGGCGTGACAGGCTGGAGACTTTGTGAAAGAATCCTGGCCtgaGT |
| | | TTGGACAAACCACAACTAGAATctgtgccttctagttgccagccatctgttgtttgcccctcccccgtgccttcctt |
| | | gaccctggaaggtgccactcccactgtcctttcctaataaaatgagaaaattgcatcgcattgtctgagtaggtgtcattctatt |
| | | ctggggggtggggtggggcaggacagcaagggggaggattgggaagacaatagcaggcatgctggggatgcggtgg |
| | | gctctatggGTctacaACTAGTagatctCACTGCCCGCccactccctctctgcgcgctcgctcgctcactgaggc |
| | | cgggcgaccaaaggtcgcccgacgcccgggctttgcccgggggcctcagtgagcgagcgagcgcgcagagaggga |
| | | tgcattaatgGGATCCtctagagtcgacctgcaggcatgcaagcttggcgtaatcatggtcatagctgtttcctgtgtgaa |
| | | attgttatccgctcacaattccacacaacatacgagccggaagcataaagtgtaaagcctggggtgcctaatgagtgagct |
| | | aactcacattaattgcgttgcgctcactgcccgctttccagtcgggaaacctgtcgtgccagctgcattaatgaatcggccaa |
| | | cgcgcggggagaggcggtttgcgtattgggcgctcttacgcttcctcgctcactgactcgctgcgctcggtcgttcggctgcg |
| | | gcgagcggtatcagctcactcaaaggcggtaatacggttatccacagaatcaggggataacgcaggaaagaacatgtg |
| | | agcaaaaggccagcaaaaggccaggaaccgtaaaaaggccgcgttgctggcgtttttccataggctccgcccccctgac |
| | | gagcatcacaaaaatcgacgctcaagtcagaggtggcgaaacccgacaggactataaagataccaggcgtttccccct |
| | | ggaagctccctcgtgcgctctcctgttccgaccctgccgcttaccggatacctgtccgcctttctcccttcgggaagcgtggcg |
| | | ctttctcatagctcacgctgtaggtatctcagttcggtgtaggtcgttcgctccaagctgggctgtgtgcacgaaccccccgttc |
| | | agcccgaccgctgcgccttatccggtaactatcgtcttgagtccaacccggtaagacacgacttatcgccactggcagcag |
| | | ccactggtaacaggattagcagagcgaggtatgtaggcggtgctacagagttcttgaagtggtggcctaactacggctaca |
| | | ctagaaggacagtatttggtatctgcgctctgctgaagccagttaccttcggaaaaagagttggtagctcttgatccggcaaa |
| | | caaaccaccgctggtagcggtggtttttttgtttgcaagcagcagattacgcgcagaaaaaaaggatctcaagaagatcctt |
| | | tgatcttttctacggggtctgacgctcagtggaacgaaaactcacgttaagggattttggtcatgagattatcaaaaaggatct |
| | | tcacctagatccttttaaattaaaaatgaagttttaaatcaatctaaagtatatatgagtaaacttggtctgacagttaccaatgc |
| | | ttaatcagtgaggcacctatctcagcgatctgtctatttcgttcatccatagttgcctgactccccgtcgtgtagataactacgat |
| | | acgggagggcttaccatctggccccagtgctgcaatgataccgcgagacccacgctcaccggctccagatttatcagcaa |
| | | taaaccagccagccggaagggccgagcgcagaagtggtcctgcaactttatccgcctccatccagtctattaattgttgccg |
| | | ggaagctagagtaagtagttcgccagttaatagtttgcgcaacgttgttgccattgctacaggcatcgtggtgtcacgctcgtc |
| | | gtttggtatggcttcattcagctccggttcccaacgatcaaggcgagttacatgatcccccatgttgtgcaaaaaagcggttag |
| | | ctccttcggtcctccgatcgttgtcagaagtaagttggccgcagtgttatcactcatggttatggcagcactgcataattctctta |
| | | ctgtcatgccatccgtaagatgcttttctgtgactggtgagtactcaaccaagtcattctgagaatagtgtatgcggcgaccga |
| | | gttgctcttgcccggcgtcaatacgggataataccgcgccacatagcagaactttaaaagtgctcatcattggaaaacgttct |
| | | tcggggcgaaaactctcaaggatcttaccgctgttgagatccagttcgatgtaacccactcgtgcacccaactgatcttcagc |
| | | atcttttactttcaccagcgtttctgggtgagcaaaaacaggaaggcaaaatgccgcaaaaaagggaataagggcgaca |
| | | cggaaatgttgaatactcatactcttcctttttcaatattattgaagcatttatcagggttattgtctcatgagcggatacatatttg |
| | | aatgtatttagaaaaataaacaaataggggttccgcgcacatttccccgaaaagtgccacctgacgtctaagaaaccattatt |
| | | atcatgacattaacctataaaaataggcgtatcacgaggccctttcgtc |

Genomic DNA was harvested 3 days after transduction. Data is shown in FIG. 24A-24C.

### 7.12. Example 12: Assessment of Integration Enzymes Encoded by Engineered mRNA

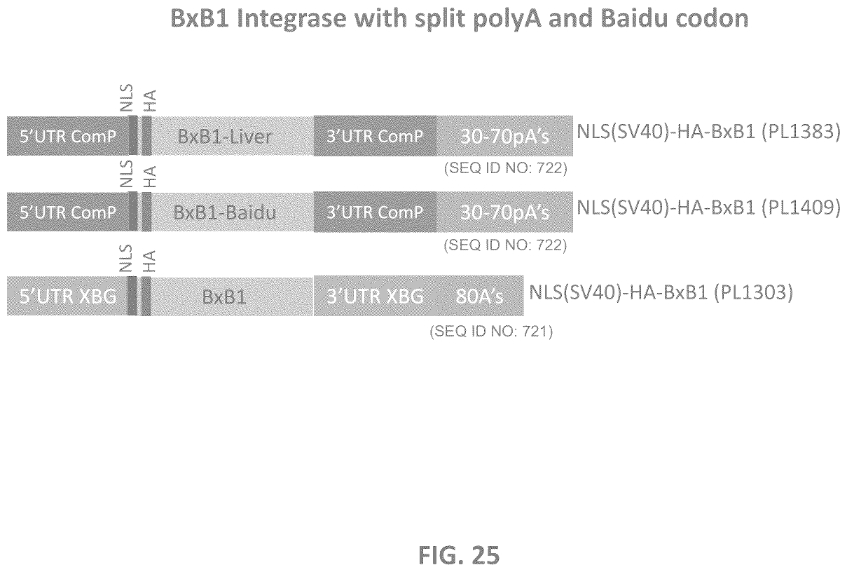

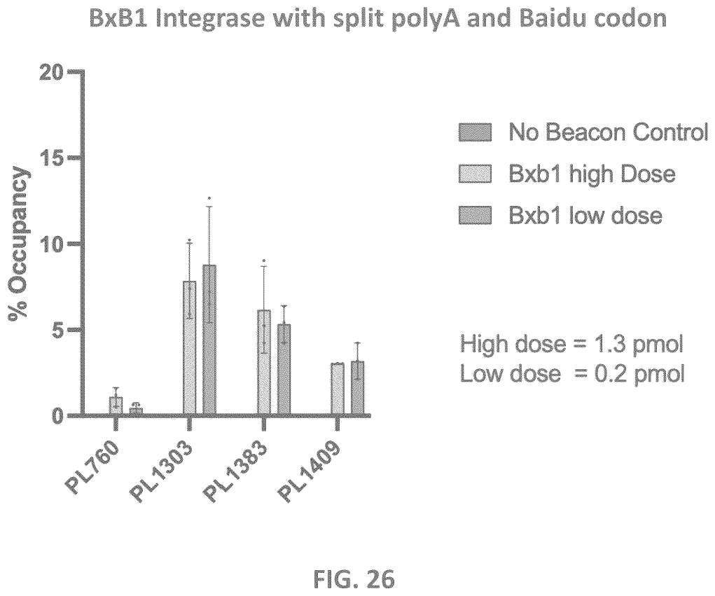

Engineered integrases described in FIG. 25 (see also Table 26) were evaluated for their ability to mediate programmable gene insertion (as indicated by “Occupancy %” (FIG. 26)).

For these experiments, the engineered integrases were introduced into a primary human hepatocyte (PHH) at a high dose (1.3 pmol) and a low dose (0.2 pmol) along with a gene editor polypeptide (i.e., mRNA encoding a gene editor polypeptide (i.e., a Cas9 nickase fused to a reverse transcriptase)); an AAV expressing an atgRNA (the atgRNA comprising a spacer sequence with sequence complementarity to a sequence in the human factor IX locus, a scaffold, an RT template comprising an integration recognition site, and a primer binding site (see Table 19 above for atgRNA sequences); and a donor polynucleotide template comprising a cognate integration recognition site. Similar to Example 11, the AAV used to deliver the atgRNA also included the donor polynucleotide template sequence. The AAV sequence is AAVG048 (SEQ ID NO: 592; see Table 20 above). Genomic DNA was harvested 3 days after transduction. Data is shown in FIG. 26.

### 7.13. Example 13: Assessment of Tag/Domain Placement in Engineered Integration Enzymes on PGI

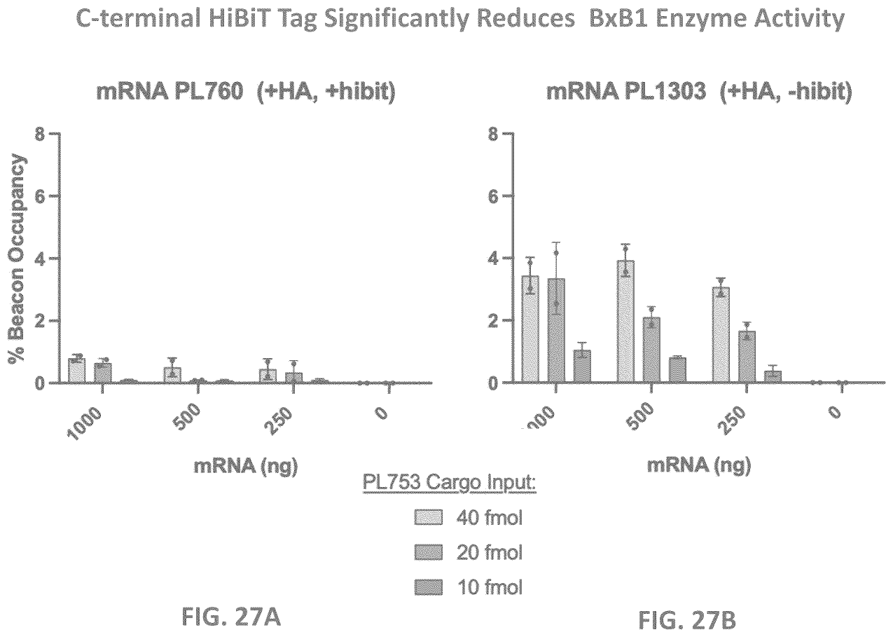

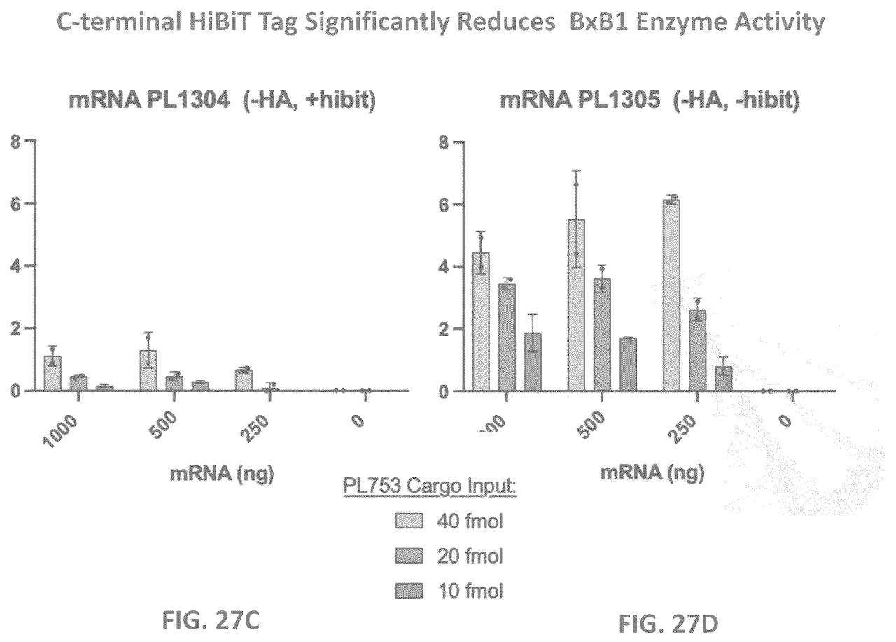

Engineered integrases described in FIG. 23 (see also Table 26) comprising were evaluated for their ability to mediate programmable gene insertion (as indicated by “Occupancy %” ˜(FIGS. 27A-27D)). In particular, additional experiments were performed to assess engineered BxB1 integrases having a c-terminal tag. In particular, these engineered integrases were assessed for their ability to mediate programmable gene insertion (e.g., as indicated by 00 Beacon Occupancy).

For these experiments, the engineered integrases were introduced into a primary human hepatocyte (PHH) at amounts of 1000 ng, 500 ng, and 250 ng, along with a gene editor polypeptide (i.e., mRNA encoding a gene editor polypeptide (i.e., a Cas9 nickase fused to a reverse transcriptase)); an atgRNA comprising a spacer sequence with sequence complementarity to a sequence in the human factor IX locus, a scaffold, an RT template comprising an integration recognition site, and a primer binding site (see Table 19 above for atgRNA sequences); and a donor polynucleotide template comprising a cognate integration recognition site at 40 fmol, 20 fmol, and 10 fmol. The donor polynucleotide template was introduced into the cells as an AAV vector (PL 753 (SEQ ID NO: 593)). Genomic DNA was harvested 3 days after transduction.

TABLE 21

| SEQ | | |
|---|---|---|
| ID | | |
| NO: | Name | Sequence |
| 593 | PL753 | ccccaactggggtaaccTTTGGGCTCCCCGGGCGCGACTATAAGCTGCGAGCAACTTCAC |
| | | TTGGGTATGCCGGCGGTAGCGCTTACCGTTCGTATAATGTATGCTATACGAAGTTATCCG |
| | | AAGCCGCTAGCGGTGGTTTGTCTGGTCAACCACCGCGGTCTCAGTGGTGTACGGTAC |
| | | AAACCCAGCTACCGGTCGTAGGTCAGTGAAGAGAAGAACAAAAAGCAGCATATTACAG |
| | | TTAGTTGTCTTCATCAATCTTGAAATATGTTGTGTCCTTTTTCTCTCCCTGTTTCCACAG |
| | | TTATGGGCAACAGCTTCAGCACCAGCGCCTTCGGCCCTGTGGCCTTTTCTCTGGGCCT |
| | | CCTGCTCGTGCTGCCTGCCGCTTTTCCAGCTCCTGTGTTCACCCTGGAAGATTTCGTG |
| | | GGAGATTGGCGGCAGACCGCCGGCTACAACCTGGACCAAGTGCTGGAACAGGGCGG |
| | | AGTGTCCAGCCTGTTTCAGAACCTGGGCGTCTCCGTGACCCCTATCCAGCGGATCGT |
| | | GCTGAGCGGCGAGAACGGCCTGAAAATCGACATCCATGTGATTATCCCCTACGAGGG |
| | | CCTGAGCGGAGATCAGATGGGCCAGATCGAGAAAATCTTCAAGGTGGTGTACCCCGT |
| | | CGACGACCACCACTTCAAGGTGATCCTGCACTACGGCACCCTGGTGATCGACGGCGT |
| | | TACCCCTAACATGATCGACTACTTCGGCAGACCCTATGAGGGAATTGCCGTGTTCGAC |
| | | GGCAAGAAAATCACCGTGACCGGCACACTGTGGAACGGCAACAAGATCATCGATGAG |
| | | CGCCTGATCAACCCAGACGGCAGCCTGCTGTTCAGAGTGACAATCAATGGCGTGACA |
| | | GGCTGGAGACTTTGTGAAAGAATCCTGGCCGGTTCTGGCGGCGCCACCAACTTCAGC |
| | | CTGCTGAAGCAGGCCGGCGACGTGGAAGAGAATCCCGGACCTGAGGGCAGAGGATC |
| | | TCTGCTGACATGCGGCGATGTGGAAGAGAATCCTGGACCTGCTATGAAAATCGAGTGC |
| | | AGAATTACAGGCACACTGAACGGAGTTGAATTCGAGCTGGTCGGCGGAGGCGAGGGC |
| | | ACACCTGAGCAGGGCAGAATGACCAACAAGATGAAAAGCACCAAGGGCGCCCTGACC |
| | | TTTTCTCCTTACCTGCTGAGCCACGTGATGGGCTATGGCTTCTACCACTTCGGCACCTA |
| | | CCCCAGCGGCTATGAAAACCCCTTCCTGCATGCTATCAACAACGGAGGCTACACCAAT |
| | | ACCAGAATCGAGAAGTACGAGGACGGCGGCGTGCTGCACGTGTCCTTCAGCTACAGA |
| | | TACGAGGCCGGCAGAGTGATCGGCGACTTCAAGGTGGTGGGCACAGGATTTCCAGAA |
| | | GATAGCGTGATCTTCACCGACAAGATCATCCGGAGCAACGCCACCGTGGAACACCTG |
| | | CACCCCATGGGCGATAATGTGCTGGTGGGCTCCTTTGCTAGAACATTCTCCCTGCGGG |
| | | ACGGCGGATACTACAGCTTCGTGGTCGACAGCCACATGCACTTCAAGTCTGCCATCCA |
| | | CCCTTCTATCCTGCAGAACGGCGGACCTATGTTCGCCTTCCGGCGGGTGGAGGAACT |
| | | CCACAGCAACACCGAGCTGGGCATCGTGGAATACCAGCACGCCTTTAAGACCCCTATC |
| | | GCCTTCGCCAGAAGCAGAGCCAGGTGAGAGCTCGATGAGTTTGGACAAACCACAACT |
| | | AGAATGCAGTGAAAAAAATGCTTTATTTGTGAAATTTGTGATGCTATTGCTTTATTTGTg |
| | | ggcccg |

Data is shown in FIGS. 27A-27D. Overall, this data showed that a c-terminal tag reduced integration efficiency of BxB1.

### 7.14. Example 14: Assessment of Integration Enzymes Engineered to Include Stabilization Domains

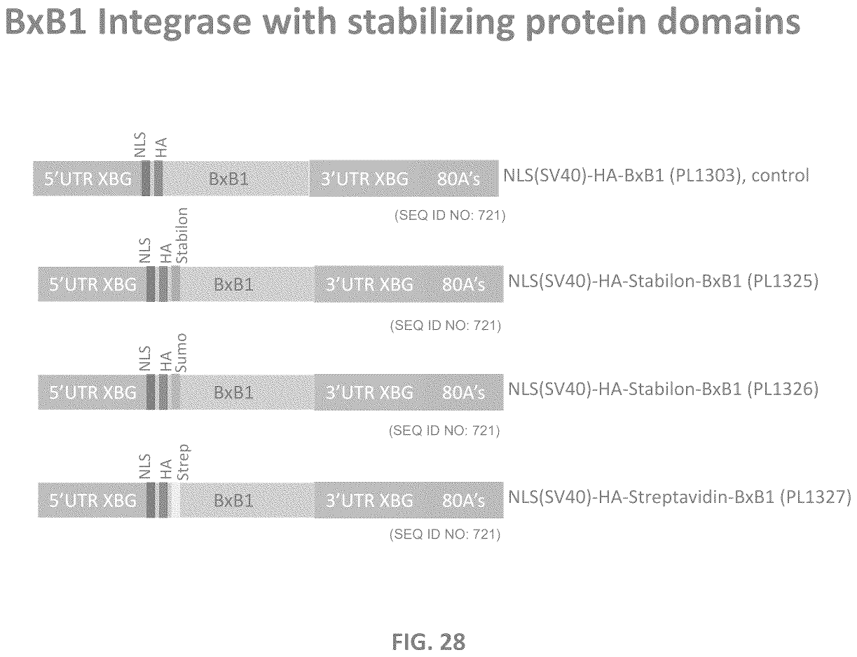

The integration enzymes engineered to include stabilization domains were also assessed for their dose dependent impact on PGI. Additional engineered integration enzymes are described in FIG. 28 (see also Table 26).

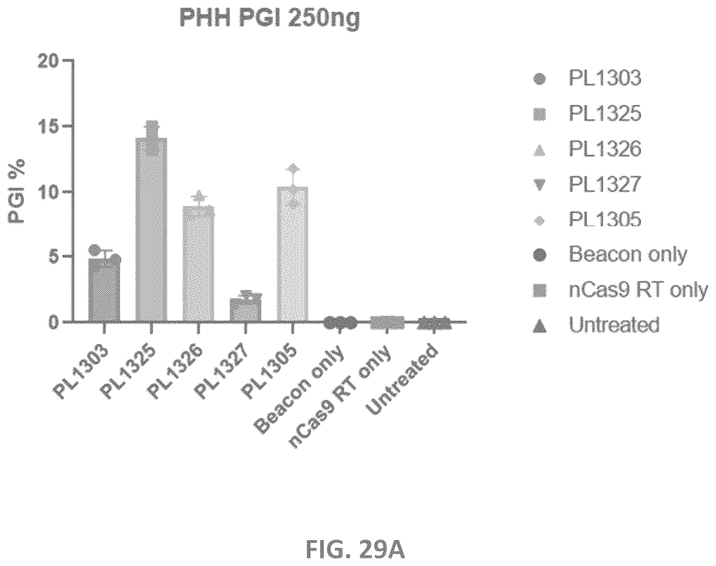

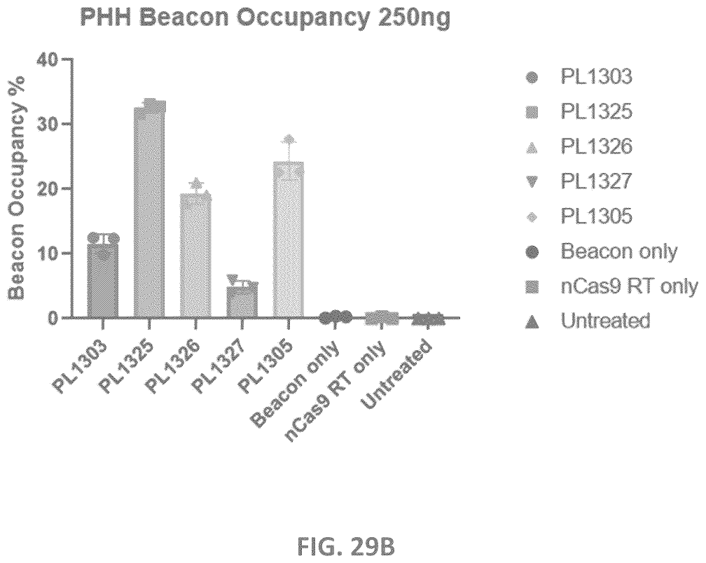

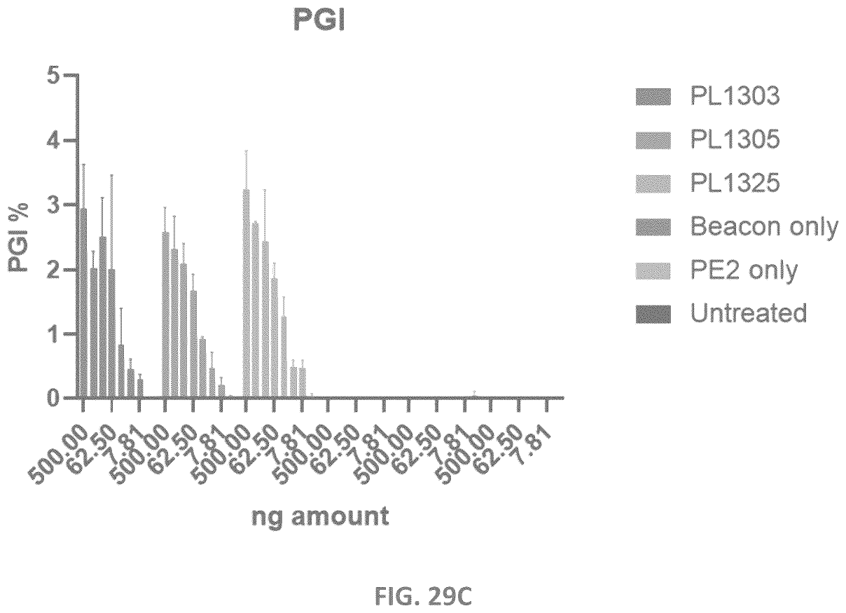

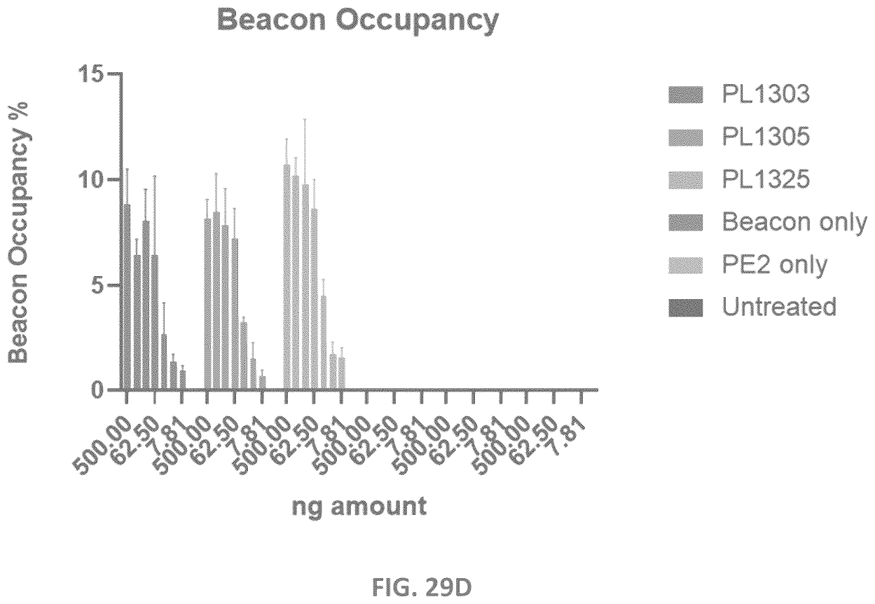

For these experiments, the engineered integrases were introduced into a primary human hepatocyte (PHH) at a 250 ng along with a gene editor polypeptide (i.e., mRNA encoding a gene editor polypeptide (i.e., a Cas9 nickase fused to a reverse transcriptase)); an atgRNA comprising a spacer sequence with sequence complementarity to a sequence in the human factor IX locus, a scaffold, an RT template comprising an integration recognition site, and a primer binding site (see Table 19 above in Example 11 for atgRNA sequences); and a donor polynucleotide template comprising a cognate integration recognition site. Genomic DNA was harvested 3 days after transduction. Data for programmable gene insertion (PGI) is shown in FIGS. 29A-29B. This data showed that an engineered BxB1 comprising a stabilon domain resulted in increased programmable gene insertion (PGI) compared to the other BxB1 polypeptides, including other engineered BxB1 polypeptides.

Additional experiments were performed to assess the engineered integrases introduced into a primary human hepatocyte (PHH) at 500 ng, 62.5 ng, and 7.81 ng along with a gene editor polypeptide (i.e., mRNA encoding a gene editor polypeptide (i.e., a Cas9 nickase fused to a reverse transcriptase)); an atgRNA comprising a spacer sequence with sequence complementarity to a sequence in the human factor IX locus, a scaffold, an RT template comprising an integration recognition site, and a primer binding site (see Table 19 above for atgRNA sequences); and a donor polynucleotide template comprising a cognate integration recognition site. Genomic DNA was harvested 3 days after transduction. Data for programmable gene insertion (PGI) is shown in FIGS. 29C-29D.

Overall, similar to the data in FIG. 29A-29B, the data in FIG. 29C-29D showed that an engineered BxB1 comprising a stabilon domain has increased programmable gene insertion (PGI) compared to the other BxB1 polypeptides, including other engineered BxB1 polypeptides.

### 7.15. Example 15: Assessment of Engineered Integration Enzymes in Pluripotent Stem Cells

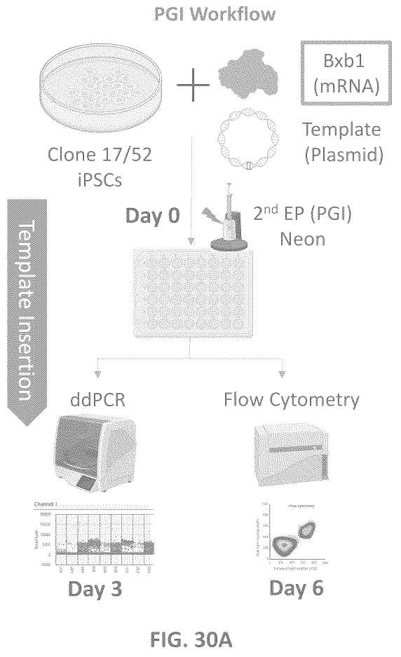

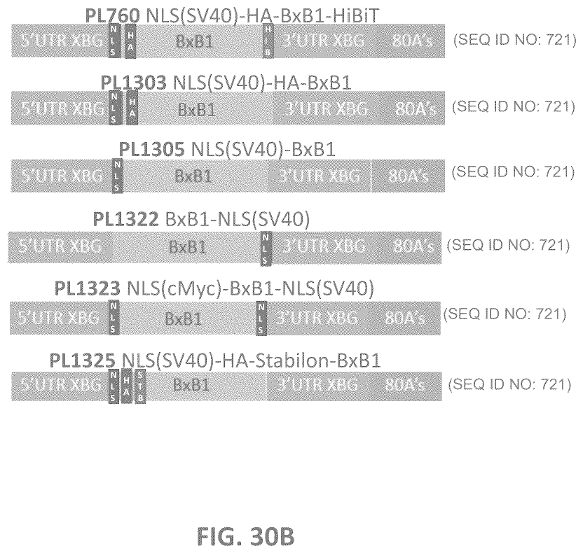

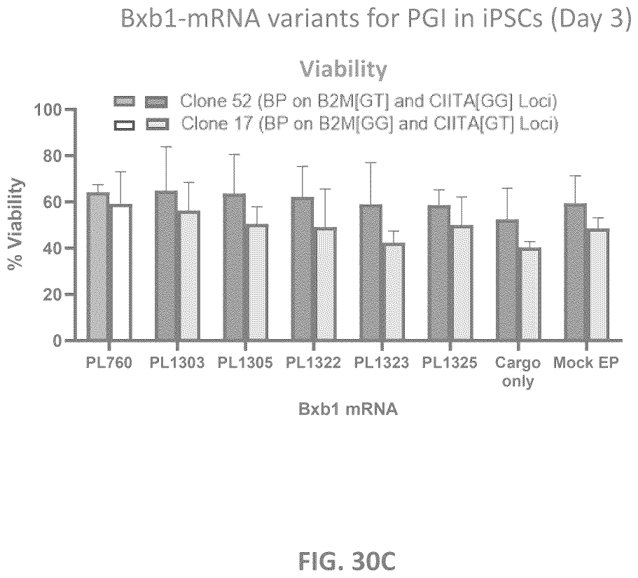

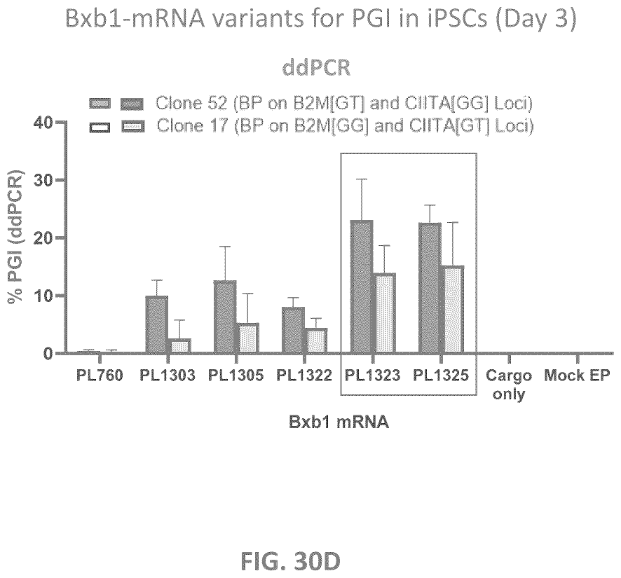

Engineered integration enzymes were assessed for their ability to mediate PGI in pluripotent stem cells. A non-limiting exemplary workflow is as described in FIG. 30A. Engineered integration enzymes that were used in these experiments are as described in FIG. 30B (see also Table 26).

For these experiments, the engineered integrases were introduced into a pluripotent stem cell clone 52 or clone 17 along with a BxB1 mRNA; and a donor polynucleotide template comprising a cognate integration recognition site (PL1113 (SEQ ID NO: 594; see Table 22 below)).

### Electroporation Conditions—

| | |
|---|---|
| Media | E8 media |
| Coating | Vitronectin |
| Cells/rxn | 1e5 cells |
| EP Program | 1200 V, 30 ms, 1 pulse |
| Recovery | E8 + CloneR2 24 h post-EP |
| Bxb1 mRNA | 2 ug/rxn |
| PL1113 [GT] | 1 ug/rxn |

TABLE 22

| SEQ | | |
|---|---|---|
| ID | | |
| NO: | Name | Sequence |
| 594 | PL1113 | tggtttttttgtttgcaagcagcagattacgcgcagaaaaaaaggatctcaagaagatcctttgatcttttctacggggtctgacg |
| | (pUC57- | ctcagtggaacgaaaactcacgttaagggattttggtcatgagattatcaaaaaggatcttcacctagatccttttaaattaaaa |
| | KAN- | atgaagttttaaatcaagcccaatctgaataatgttacaaccaattaaccaattctgattagaaaaactcatcgagcatcaaat |
| | EF1a- | gaaactgcaatttattcatatcaggattatcaataccatatttttgaaaaagccgtttctgtaatgaaggagaaaactcaccgag |
| | GFP- | tcaaaaataaggttatcaagtgagaaatcaccatgagtgacgactgaatccggtgagaatggcaaaagtttatgcatttctttc |
| | P2A- | ccagcgcatcaacaatattttcacctgaatcaggatattcttctaatacctggaatgctgtttttccggggatcgcagtggtgagt |
| | Puro) | cagacttgttcaacaggccagccattacgctcgtcatcaaaatcactcgcatcaaccaaaccgttattcattcgtgattgcgcct |
| | | gagcgagacgaaatacgcgatcgctgttaaaaggacaattacaaacaggaatcgaatgcaaccggcgcaggaacactg |
| | | gcagttccataggatggcaagatcctggtatcggtctgcgattccgactcgtccaacatcaatacaacctattaatttcccctcg |
| | | aaccatgcatcatcaggagtacggataaaatgcttgatggtcggaagaggcataaattccgtcagccagtttagtctgaccat |
| | | ctcatctgtaacatcattggcaacgctacctttgccatgtttcagaaacaactctggcgcatcgggcttcccatacaagcgata |
| | | gattgtcgcacctgattgcccgacattatcgcgagcccatttatacccatataaatcagcatccatgttggaatttaatcgcggc |
| | | ctcgacgtttcccgttgaatatggctcataacaccccttgtattactgtttatgtaagcagacagttttattgttcatgatgatatattttt |
| | | atcttgtgcaatgtaacatcagagattttgagacacgggccagagctgcatcgcgcgtttcggtgatgacggtgaaaacctct |
| | | gacacatgcagctcccggagacggtcacagcttgtctgtaagcggatgccgggagcagacaagcccgtcagggcgcgtc |
| | | agcgggtgttggcgggtgtcggggctggcttaactatgcggcatcagagcagattgtactgagagtgcaccatatgcggtgt |
| | | gaaataccgcacagatgcgtaaggagaaaataccgcatcaggcgccattcgccattcaggctgcgcaactgttgggaag |
| | | ggcgatcggtgcgggcctcttcgctattacgccagctggcgaaagggggatgtgctgcaaggcgattaagttgggtaacgc |
| | | cagggttttcccagtcacacgttgtaaaacgacggccagaGTGGTTTGTCTGGTCAACCACCGCGGTCT |
| | | CAGTGGTGTACGGTACAAACCCAgaattcgagctcggctccggtgcccgtcagtgggcagagcgcacatc |
| | | gcccacagtccccgagaagttggggggaggggtcggcaattgaaccggtgcctagagaaggtggcgcggggtaaactg |
| | | ggaaagtgatgtcgtgtactggctccgcctttttcccgagggtgggggagaaccgtatataagtgcagtagtcgccgtgaacg |
| | | ttctttttcgcaacgggtttgccgccagaacacaggtaagtgccgtgtgtggttcccgcgggcctggcctctttacgggttatggc |
| | | ccttgcgtgccttgaattacttccacctggctgcagtacgtgattcttgatcccgagcttcgggttggaagtggggggagagttc |
| | | gaggccttgcgcttaaggagccccttcgcctcgtgcttgagttgaggcctggcctgggcgctggggccgccgcgtgcgaatct |
| | | ggtggcaccttcgcgcctgtctcgctgctttcgataagtctctagccatttaaaatttttgatgacctgctgcgacgctttttttctggc |
| | | aagatagtcttgtaaatgcgggccaagatctgcacactggtatttcggtttttggggccgcgggcggcgacggggcccgtgc |
| | | gtcccagcgcacatgttcggcgaggcggggcctgcgagcgcggccaccgagaatcggacgggggtagtctcaagctgg |
| | | ccggcctgctctggtgcctggcctcgcgccgccgtgtatcgccccgccctgggcggcaaggctggcccggtcggcaccagt |
| | | tgcgtgagcggaaagatggccgcttcccggccctgctgcagggagctcaaaatggaggacgcggcgctcgggagagcg |
| | | ggcgggtgagtcacccacacaaaggaaaagggcctttccgtcctcagccgtcgcttcatgtgactccacggagtaccgggc |
| | | gccgtccaggcacctcgattagttctcgagcttttggagtacgtcgtctttaggttggggggaggggttttatgcgatggagtttcc |
| | | ccacactgagtgggtggagactgaagttaggccagcttggcacttgatgtaattctccttggaatttgccctttttgagtttggatct |
| | | tggttcattctcaagcctcagacagtggttcaaagtttttttcttccatttcaggtgtcgtgaGGATCCgccaccatggtcagca |
| | | agggcgaggaactgttcaccggggtggtgcccatcctggtcgagctggacggcgacgtaaacggccacaagttcagcgtg |
| | | tccggcgagggcgagggcgatgccacctacggcaagctgaccctgaagttcatctgtaccaccggcaagctgcccgtgcc |
| | | ctggcccaccctcgtgaccaccctgacctacggcgtgcaatgcttcagccgctaccccgaccacatgaagcagcacgactt |
| | | cttcaagtccgccatgcccgaaggctacgtccaggagcgcaccatcttcttcaaggacgacggcaactacaagacccgcg |
| | | ccgaggtgaagttcgagggcgacaccctggtgaaccgcatcgagctgaagggcatcgacttcaaggaggacggcaaca |
| | | tcctggggcacaagctggagtacaactacaacagccacaacgtctatatcatggccgacaagcagaagaacggcatcaa |
| | | ggtgaacttcaagatccgccacaacatcgaggacggcagcgtgcaactcgccgaccactaccagcagaacacccccat |
| | | cggcgacggccccgtgctgctgcccgacaaccactacctgagcacccagtccgccctgagcaaagaccccaacgagaa |
| | | gcgcgatcacatggtcctgctggagttcgtgaccgccgccgggatcactctcggcatggacgagctgtacaagGGAAG |
| | | CGGAGCTACTAACTTCAGCCTGCTGAAGCAGGCTGGCGACGTGGAGGAGAACCCT |
| | | GGACCTATGGTTACAGAGTATAAACCTACGGTCCGACTCGCAACGAGAGATGATGT |
| | | GCCCCGCGCGGTTCGCACACTTGCGGCAGCTTTTGCTGACTATCCCGCGACTCGGC |
| | | ATACGGTCGATCCGGACCGACATATCGAGCGCGTAACAGAACTTCAAGAACTCTTTC |
| | | TCACAAGAGTTGGACTTGACATTGGGAAGGTATGGGTAGCCGATGACGGTGCGGCT |
| | | GTTGCGGTATGGACAACACCCGAAAGTGTAGAGGCAGGCGCGGTCTTCGCTGAGAT |
| | | CGGTCCGCGGATGGCCGAACTGTCCGGTTCACGATTGGCGGCGCAACAACAAATG |
| | | GAGGGTCTCCTCGCTCCACATAGACCTAAAGAGCCAGCGTGGTTTCTTGCCACAGT |
| | | GGGCGTTAGCCCCGACCACCAGGGTAAGGGACTTGGGAGCGCAGTTGTATTGCCA |
| | | GGAGTTGAAGCGGCCGAGAGGGCGGGCGTACCAGCGTTCCTTGAGACATCTGCGC |
| | | CCCGCAACCTGCCCTTTTACGAACGCCTCGGATTCACCGTCACCGCCGATGTAGAG |
| | | GTCCCAGAAGGTCCCAGAACCTGGTGCATGACCCGGAAACCCGGGGCGTGAgtttaaa |
| | | cccgcAAGCTTgcctcgactgtgccttctagttgccagccatctgttgtttgcccctcccccgtgccttccttgaccctggaag |
| | | gtgccactcccactgtcctttcctaataaaatgagaaaattgcatcgcattgtctgagtaggtgtcattctattctggggggtggg |
| | | gtggggcaggacagcaagggggaggattgggaagacaatagcaggcatgctggggatgcggtgggctctatggtcttcc |
| | | gcttcctcgctcactgactcgctgcgctcggtcgttcggctgcggcgagcggtatcagctcactcaaaggcggtaatacggtta |
| | | tccacagaatcaggggataacgcaggaaagaacatgtgagcaaaaggccagcaaaaggccaggaaccgtaaaaag |
| | | gccgcgttgctggcgtttttccataggctccgcccccctgacgagcatcacaaaaatcgacgctcaagtcagaggtggcgaa |
| | | acccgacaggactataaagataccaggcgtttccccctggaagctccctcgtgcgctctcctgttccgaccctgccgcttacc |
| | | ggatacctgtccgcctttctcccttcgggaagcgtggcgctttctcatagctcacgctgtaggtatctcagttcggtgtaggtcgtt |
| | | cgctccaagctgggctgtgtgcacgaaccccccgttcagcccgaccgctgcgccttatccggtaactatcgtcttgagtccaa |
| | | cccggtaagacacgacttatcgccactggcagcagccactggtaacaggattagcagagcgaggtatgtaggcggtgcta |
| | | cagagttcttgaagtggtggcctaactacggctacactagaagaacagtatttggtatctgcgctctgctgaagccagttacctt |
| | | cggaaaaagagttggtagctcttgatccggcaaacaaaccaccgctggtagcgg |

Genomic DNA was harvested 3 days after transduction. Data for cell viability is shown in FIG. 30C. Data for programmable gene insertion (PGI) is shown in FIG. 30D. As shown in FIG. 30D shows that PL1323 and PL1325 yield up to 30% PGI in Clone 52 and up to 20% in Clone 17.

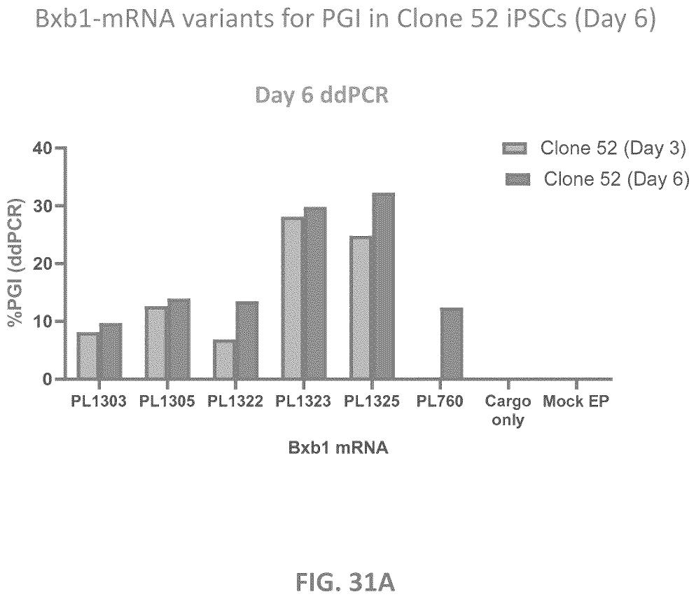

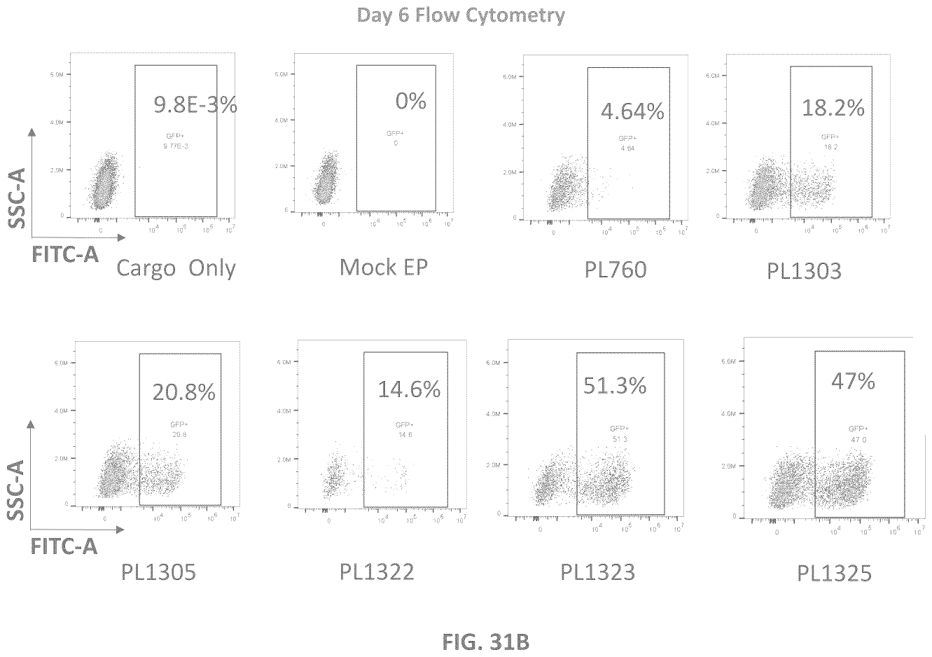

The PGI in pluripotent stem cells was also evaluated at day 6 after transduction. FIG. 31A shows ddPCR data for PGI for clone 52 at day 3 and day 6. Flow cytometry was used to assess GFP expression which indicates programmable gene insertion (PGI) (see FIG. 31B). This data shows that PL1323 and PL1325 yield % GFP+ cells by flow ˜50%.

### 7.16. Example 16: Assessment of Engineered Integration Enzyme in Hematopoietic Stem Cells

Engineered integrases were also assessed for their ability to mediate programmable gene insertion in hematopoietic stem cells.

For these experiments, the engineered integrases were introduced into a hematopoietic stem cell along with a gene editor polypeptide (i.e., mRNA encoding a gene editor polypeptide (i.e., a Cas9 nickase fused to a reverse transcriptase)); an atgRNA comprising a spacer sequence a scaffold, an RT template comprising an integration recognition site, and a primer binding site; and a donor polynucleotide template comprising a cognate integration recognition site. AtgRNA sequences used in this Example are provided in Table 23 below.

TABLE 23

| SEQ ID | atgRNA | |
|---|---|---|
| NO: | pair name | atgRNA sequence with modifications (5′-3′) |
| 595 | hB2M-AA- | mU*mC*mC*rUrGrArArGrCrUrGrArCrArGrCrArUrUrCrGrUrUrUrUrArGrAmGmCmUm |
| | 1-GT | AmGmAmAmAmUmAmGmCrArArGrUrUrArArArArUrArArGrGrCrUrArGrUrCrCrGrUrU |
| | bmp002 | rArUrCrAmAmCmUmUmGmAmAmAmAmAmGmUmGmGmCmAmCmCmGmAmGmU |
| | | mCmGmGmUmGmCrArUrGrArUrCrCrUrGrArCrGrArCrGrGrArGrArCrCrGrCrCrGrUrC |
| | | rGrUrCrGrArCrArArGrCrCrCrUrGrCrUrGrUrCrArGrCrUrUrCmU*mU*mU*mU |
| 596 | hB2M-AA- | mG*mA*mG*rGrArArGrGrArCrCrArGrArGrCrGrGrGrArGrUrUrUrUrArGrAmGmCmUm |
| | 1-GT atg- | AmGmAmAmAmUmAmGmCrArArGrUrUrArArArArUrArArGrGrCrUrArGrUrCrCrGrUrU |
| | R bmp002 | rArUrCrAmAmCmUmUmGmAmAmAmAmAmGmUmGmGmCmAmCmCmGmAmGmU |
| | | mCmGmGmUmGmCrGrGrCrUrUrGrUrCrGrArCrGrArCrGrGrCrGrGrUrCrUrCrCrGrUr |
| | | CrGrUrCrArGrGrArUrCrArUrCrGrCrUrCrUrGrGrUrCrCrUrUmU*mU*mU*mU |
| 597 | hB2M-1- | mU*mC*mC*rUrGrArArGrCrUrGrArCrArGrCrArUrUrCrGrUrUrUrUrArGrArGrCrUrArGr |
| | 38-20-atg- | ArArArUrArGrCrArArGrUrUrArArArArUrArArGrGrCrUrArGrUrCrCrGrUrUrArUrCrArArC |
| | F bmp001 | rUrUrGrArArArArArGrUrGrGrCrArCrCrGrArGrUrCrGrGrUrGrCrArCrGrArCrGrGrArGrA |
| | | rCrCrGrCrCrGrUrCrGrUrCrGrArCrArArGrCrCrUrGrCrUrGrUrCrArGrC*mU*mU*mC |
| 598 | hB2M-1- | mG*mA*mG*rGrArArGrGrArCrCrArGrArGrCrGrGrGrArGrUrUrUrUrArGrArGrCrUrArGr |
| | 38-20-atg- | ArArArUrArGrCrArArGrUrUrArArArArUrArArGrGrCrUrArGrUrCrCrGrUrUrArUrCrArArC |
| | R bmp001 | rUrUrGrArArArArArGrUrGrGrCrArCrCrGrArGrUrCrGrGrUrGrCrArCrGrArCrGrGrCrGr |
| | | GrUrCrUrCrCrGrUrCrGrUrCrArGrGrArUrCrArUrCrGrCrUrCrUrGrGrUrC*mC*mU*mU |
| 599 | hCIITA- | mU*mC*mA*rArCrUrGrCrGrArCrCrArGrUrUrCrArGrCrGrUrUrUrUrArGrAmGmCmUm |
| | AA-3-GG | AmGmAmAmAmUmAmGmCrArArGrUrUrArArArArUrArArGrGrCrUrArGrUrCrCrGrUrU |
| | atg-F | rArUrCrAmAmCmUmUmGmAmAmAmAmAmGmUmGmGmCmAmCmCmGmAmGmU |
| | bmp002 | mCmGmGmUmGmCrArUrGrArUrCrCrUrGrArCrGrArCrGrGrArGrCrCrCrGrCrCrGrUrC |
| | | rGrUrCrGrArCrArArGrCrCrCrArUrUrArGrArArCrUrGrGrUrCrGrCrArGmU*mU*mU*mU |
| 600 | hCIITA- | mG*mA*mU*rArUrUrGrGrCrArUrArArGrCrCrUrCrCrCrGrUrUrUrUrArGrAmGmCmUm |
| | AA-3-GG | AmGmAmAmAmUmAmGmCrArArGrUrUrArArArArUrArArGrGrCrUrArGrUrCrCrGrUrU |
| | atg-R | rArUrCrAmAmCmUmUmGmAmAmAmAmAmGmUmGmGmCmAmCmCmGmAmGmU |
| | bmp002 | mCmGmGmUmGmCrGrGrCrUrUrGrUrCrGrArCrGrArCrGrGrCrGrGrGrCrUrCrCrGrUr |
| | | CrGrUrCrArGrGrArUrCrArUrArGrGrCrUrUrArUrGrCrCrArAmU*mU*mU*mU |
| 601 | hAAVS1- | mG*mG*mG*rGrCrCrArCrUrArGrGrGrArCrArGrGrArUrGrUrUrUrUrArGrAmGmCmUm |
| | AA-2 atg- | AmGmAmAmAmUmAmGmCrArArGrUrUrArArArArUrArArGrGrCrUrArGrUrCrCrGrUrU |
| | F bmp002 | rArUrCrAmAmCmUmUmGmAmAmAmAmAmGmUmGmGmCmAmCmCmGmAmGmU |
| | | mCmGmGmUmGmCrArUrGrArUrCrCrUrGrArCrGrArCrGrGrArGrGrUrCrGrCrCrGrUrC |
| | | rGrUrCrGrArCrArArGrCrCrCrCrUrGrUrCrCrCrUrArGrUrGrGmU*mU*mU*mU |
| 602 | hAAVS1- | mA*mG*mA*rCrCrCrArArUrArUrCrArGrGrArGrArCrUrGrUrUrUrUrArGrAmGmCmUmA |
| | AA-2 atg- | mGmAmAmAmUmAmGmCrArArGrUrUrArArArArUrArArGrGrCrUrArGrUrCrCrGrUrUr |
| | R bmp002 | ArUrCrAmAmCmUmUmGmAmAmAmAmAmGmUmGmGmCmAmCmCmGmAmGmU |
| | | mCmGmGmUmGmCrGrGrCrUrUrGrUrCrGrArCrGrArCrGrGrCrGrArCrCrUrCrCrGrUr |
| | | CrGrUrCrArGrGrArUrCrArUrCrUrCrCrUrGrArUrArUrUrGrGmU*mU*mU*mU |
| 603 | CXCR4 | mC*mA*mU*rCrUrUrUrGrCrCrArArCrGrUrCrArGrUrGrGrUrUrUrUrArGrArGrCrUrArGr |
| | 38-38 atg- | ArArArUrArGrCrArArGrUrUrArArArArUrArArGrGrCrUrArGrUrCrCrGrUrUrArUrCrArArC |
| | F end mod | rUrUrGrArArArArArGrUrGrGrCrArCrCrGrArGrUrCrGrGrUrGrCrArUrGrArUrCrCrUrGrA |
| | | rCrGrArCrGrGrArGrArCrCrGrCrCrGrUrCrGrUrCrGrArCrArArGrCrCrUrGrArCrGrUrUr |
| | | GrGrC*mA*mA*mA |
| 604 | CXCR4 | mC*mA*mA*rCrCrArCrCrCrArCrArArGrUrCrArUrUrGrGrUrUrUrUrArGrArGrCrUrArGr |
| | 38-38 atg- | ArArArUrArGrCrArArGrUrUrArArArArUrArArGrGrCrUrArGrUrCrCrGrUrUrArUrCrArArC |
| | Rend | rUrUrGrArArArArArGrUrGrGrCrArCrCrGrArGrUrCrGrGrUrGrCrGrGrCrUrUrGrUrCrGr |
| | mod | ArCrGrArCrGrGrCrGrGrUrCrUrCrCrGrUrCrGrUrCrArGrGrArUrCrArUrUrGrArCrUrUrG |
| | | rUrGrG*mG*mU*mG |
| 605 | CXCR4 | mC*mA*mU*rCrUrUrUrGrCrCrArArCrGrUrCrArGrUrGrGrUrUrUrUrArGrArGrCrUrArGr |
| | dual | ArArArUrArGrCrArArGrUrUrArArArArUrArArGrGrCrUrArGrUrCrCrGrUrUrArUrCrArArC |
| | atgRNA | rUrUrGrArArArArArGrUrGrGrCrArCrCrGrArGrUrCrGrGrUrGrCrArCrGrArCrGrGrArGrA |
| | Fw-AttB38 | rCrCrGrCrCrGrUrCrGrUrCrGrArCrArArGrCrCrUrGrArCrGrUrUrGrGrC*mA*mA*mA |
| | 20 end | |
| | mod | |
| 606 | CXCR4 | mC*mA*mA*rCrCrArCrCrCrArCrArArGrUrCrArUrUrGrGrUrUrUrUrArGrArGrCrUrArGr |
| | dual | ArArArUrArGrCrArArGrUrUrArArArArUrArArGrGrCrUrArGrUrCrCrGrUrUrArUrCrArArC |
| | atgRNA | rUrUrGrArArArArArGrUrGrGrCrArCrCrGrArGrUrCrGrGrUrGrCrArCrGrArCrGrGrCrGr |
| | Rv-AttB38 | GrUrCrUrCrCrGrUrCrGrUrCrArGrGrArUrCrArUrUrGrArCrUrUrGrUrGrG*mG*mU*mG |
| | 20 end | |
| | mod | |
| 607 | TRAC-AM | mU*mG*mG*rCrArUrCrUrGrGrArCrUrCrCrArGrCrCrUrGrUrUrUrUrArGrAmGmCmUm |
| | atg-F | AmGmAmAmAmUmAmGmCrArArGrUrUrArArArArUrArArGrGrCrUrArGrUrCrCrGrUrU |
| | bmp002 | rArUrCrAmAmCmUmUmGmAmAmAmAmAmGmUmGmGmCmAmCmCmGmAmGmU |
| | | mCmGmGmUmGmCrArUrGrArUrCrCrUrGrArCrGrArCrGrGrArGrArGrCrGrCrCrGrUrC |
| | | rGrUrCrGrArCrArArGrCrCrCrCrUrGrGrArGrUrCrCrArGrArUmU*mU*mU*mU |
| 608 | TRAC-AM | mA*mG*mA*rGrUrCrUrCrUrCrArGrCrUrGrGrUrArCrArGrUrUrUrUrArGrAmGmCmUm |
| | atg-R | AmGmAmAmAmUmAmGmCrArArGrUrUrArArArArUrArArGrGrCrUrArGrUrCrCrGrUrU |
| | bmp002 | rArUrCrAmAmCmUmUmGmAmAmAmAmAmGmUmGmGmCmAmCmCmGmAmGmU |
| | | mCmGmGmUmGmCrGrGrCrUrUrGrUrCrGrArCrGrArCrGrGrCrGrCrUrCrUrCrCrGrUr |
| | | CrGrUrCrArGrGrArUrCrArUrArCrCrArGrCrUrGrArGrArGrAmU*mU*mU*mU |
| 609 | TRAC | mU*mG*mG*rCrArUrCrUrGrGrArCrUrCrCrArGrCrCrUrGrUrUrUrUrArGrArGrCrUrArGr |
| | dual | ArArArUrArGrCrArArGrUrUrArArArArUrArArGrGrCrUrArGrUrCrCrGrUrUrArUrCrArArC |
| | atgRNA | rUrUrGrArArArArArGrUrGrGrCrArCrCrGrArGrUrCrGrGrUrGrCrArCrGrArCrGrGrArGrA |
| | Fw-AttB38 | rCrCrGrCrCrGrUrCrGrUrCrGrArCrArArGrCrCrCrUrGrGrArGrUrCrCrA*mG*mA*mU |
| | 20 end | |
| | mod | |
| 610 | TRAC | mA*mG*mA*rGrUrCrUrCrUrCrArGrCrUrGrGrUrArCrArGrUrUrUrUrArGrArGrCrUrArGr |
| | dual | ArArArUrArGrCrArArGrUrUrArArArArUrArArGrGrCrUrArGrUrCrCrGrUrUrArUrCrArArC |
| | atgRNA | rUrUrGrArArArArArGrUrGrGrCrArCrCrGrArGrUrCrGrGrUrGrCrArCrGrArCrGrGrCrGr |
| | Rv-AM- | GrUrCrUrCrCrGrUrCrGrUrCrArGrGrArUrCrArUrArCrCrArGrCrUrGrArG*mA*mG*mA |
| | AttB38 20 | |
| | end mod | |

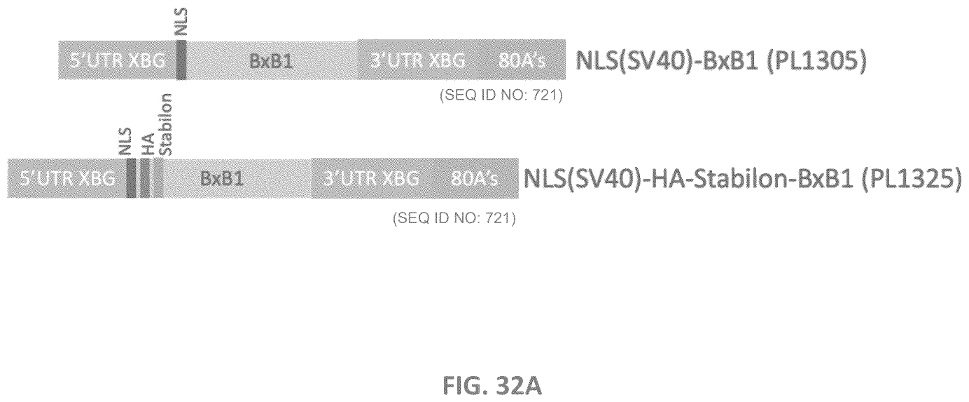

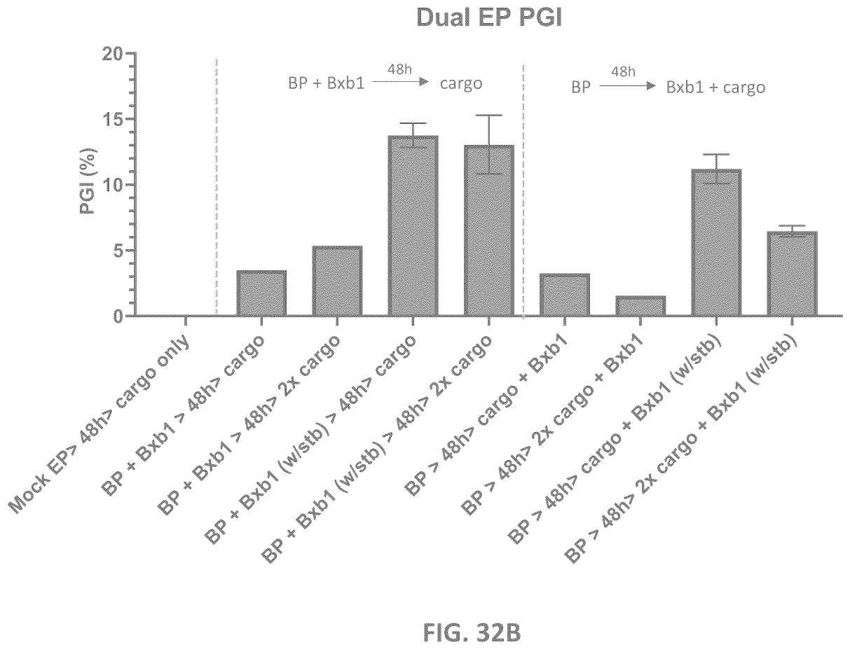

In these experiments, a dual electroporation approach was used. In one experimental condition, the engineered integrase, the gene editor polypeptide (i.e., mRNA encoding a gene editor polypeptide), and the atgRNA were introduced into a cell using a first electroporation and the donor polynucleotide template was introduced into the cell using a second electroporation. In a second experimental condition, the gene editor polypeptide (i.e., mRNA encoding a gene editor polypeptide), and the atgRNA were introduced into a cell using a first electroporation and the engineered integrase and the donor polynucleotide template (mini circle) were introduced into the cell using a second electroporation. Engineered integrases used in these experiments are as shown in FIG. 32A.

Genomic DNA was harvested 3 days after transduction. Data for programmable gene insertion (PGI) is shown in FIG. 32B. This data shows that a BxB1 integrase engineered to include a stabilon motif improves PGI by 3-5 fold.

### 7.17. Example 17: Assessment of Additional Engineered Integration Enzymes

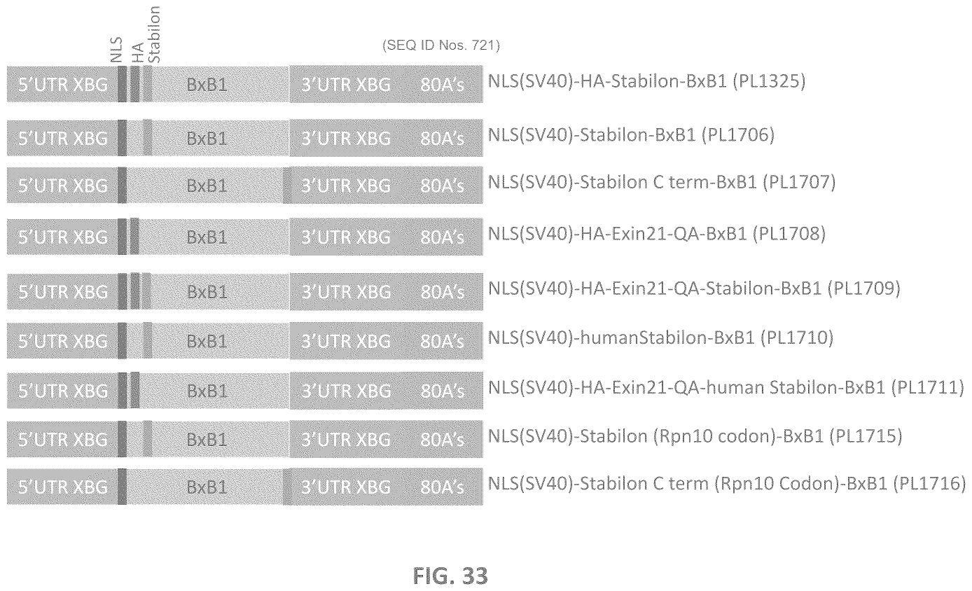

Additional iterations of engineered integration enzymes were tested for PGI (measured as beacon occupancy). In particular, BxB1 was engineered to include a stabilon domain or an Exin21 domain (see, e.g., engineered integration enzymes shown in FIG. 33 (see also Table 26)).

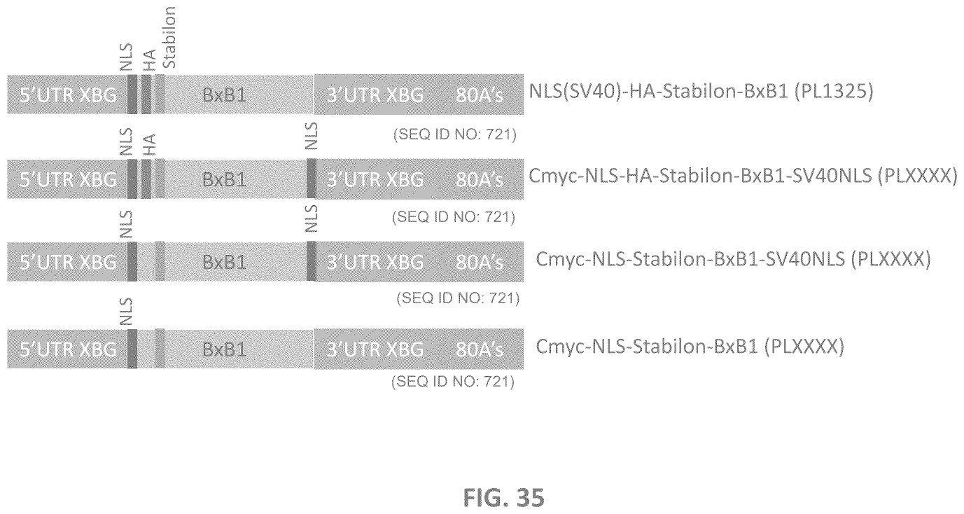

For these experiments, the engineered integration enzymes described in FIG. 33 were introduced into a HEK293 cell along with a gene editor polypeptide (i.e., mRNA encoding a gene editor polypeptide (i.e., a Cas9 nickase fused to a reverse transcriptase)); an atgRNA comprising a spacer sequence with sequence complementarity to a sequence in the human factor IX locus, a scaffold, an RT template comprising an integration recognition site, and a primer binding site (see Table 19 above for atgRNA sequences); and a donor polynucleotide template comprising a cognate integration recognition site. The data is shown in FIG. 34. The sub-set of engineered BxB1 integrases shown in FIG. 35 (see also Table 26) were selected for further study.

### 7.18. Example 18: Assessment of Engineered Integration Enzymes Comprising an Amino Acid Modification in a Degron Motif

BxBT integrases were engineered to include an amino acid modification with the aim that the modification would improve stability (e.g., half-life). FIG. 36A shows degron motifs and lysine residues that are candidate for amino acid modifications that may increase the stability of a BxBT integrase.

In these experiments, a BxBT was engineered to substitute a lysine for an arginine at position 10. Half-life of the integrase was assessed 24 and 48 hours after transduction into the cell using western blot (see FIG. 36B). This data showed that a specific lysine to arginine substitution (K10R) did not alter the stability of BxBT. Further assessment is ongoing to determine whether substituting another lysine residue (see, e.g., FIG. 36A) would be sufficient to increase the stability of the BxBT integrase.

### 7.19. Example 19: Assessment of mRNA Optimization to Increase Stability of mRNA Encoding BxB1

In another set of experiments, mRNAs encoding BxB1 were optimized with the aim of improving stability (i.e., half-life of the mRNA) upon being introduced into a cell.

In these experiments, the mRNA encoding BxB1 was optimized using the LinearDesign algorithm described in Zhang et al. (Algorithm for Optimized mRNA Design Improves Stability and Immunogenicity, Nature 2023, doi.org/10.1038/s41586-023-06127-z), which is hereby incorporated by reference in its entirety. LinearDesign optimizes both structural stability via second structure and codon usage.

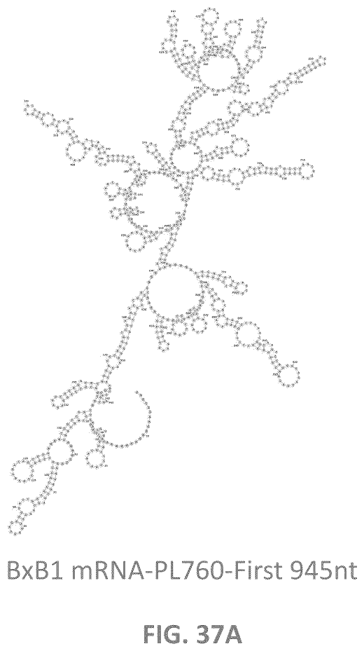

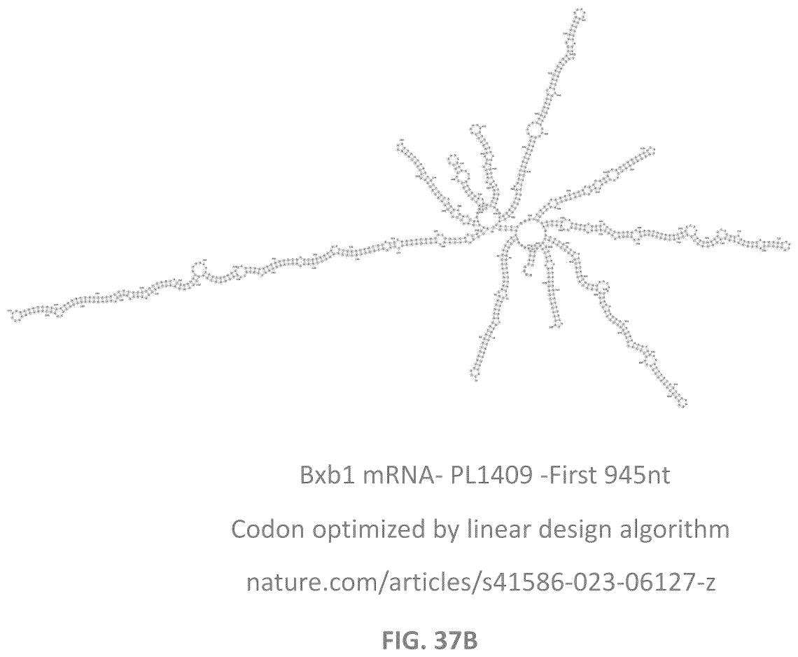

FIGS. 37A-37B show first generations attempts at optimizing BxBT RNA structure using the linear design algorithm comparing non-optimized mRNA encoding BxB1 in FIG. 37A with optimized mRNA encoding BxBT in FIG. 37B.

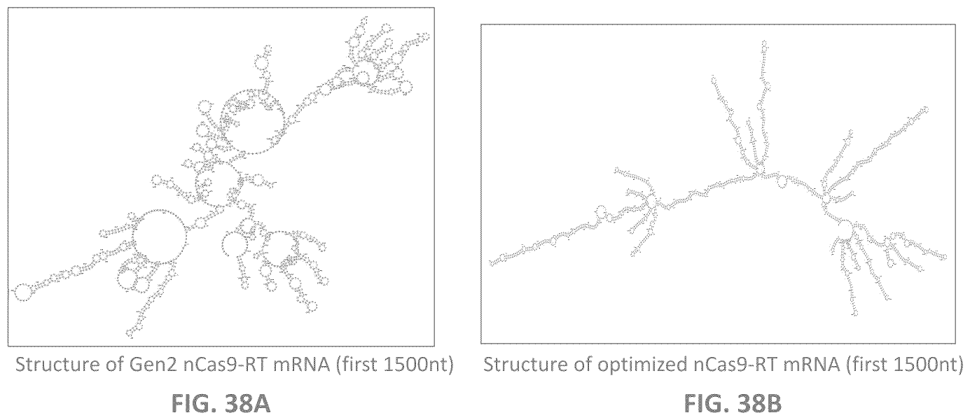

FIGS. 38A-38B show first generations attempts at optimizing an RNA structure for RNA encoding nCas9-RT using the LinearDesign algorithm comparing non-optimized mRNA encoding nCas9-RT in FIG. 38A with optimized mRNA encoding nCsa9-RT in FIG. 38B.

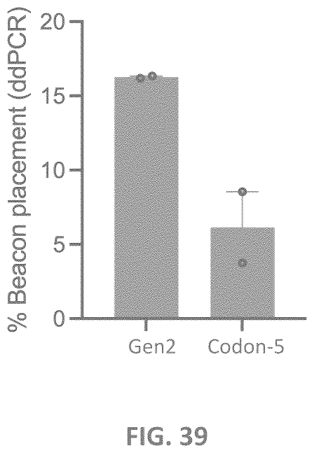

The nCas9-RTs in FIGS. 38A-38B, were tested for their ability to insert integration recognition sites into a genome at the human factor IX locus. In particular, the nCas9-RTs in FIGS. 38A-38B were introduced into a cell along with an atgRNA comprising a spacer sequence with sequence complementarity to a sequence in the human factor IX locus, a scaffold, an RT template comprising an integration recognition site, and a primer binding site (see Table 19 above in Example 11 for atgRNA sequences). Beacon placement data is shown in FIG. 39. The expectation was that the LinearDesign codon optimized mRNA would have a longer half-life due in at least in part to the second structure—predominately folded, which should lead to higher beacon placement. However, the linear design approach and the algorithm predicted codons didn't perform as anticipated.

### 7.20. Example 20: Assessment of Fusion Proteins Comprising Engineered Integration Enzymes and a Gene Editor Polypeptide

Fusion proteins comprising an nCas9-RT are fused with engineered integration enzymes (e.g, BxB1) and tested for their ability to mediate PGI.

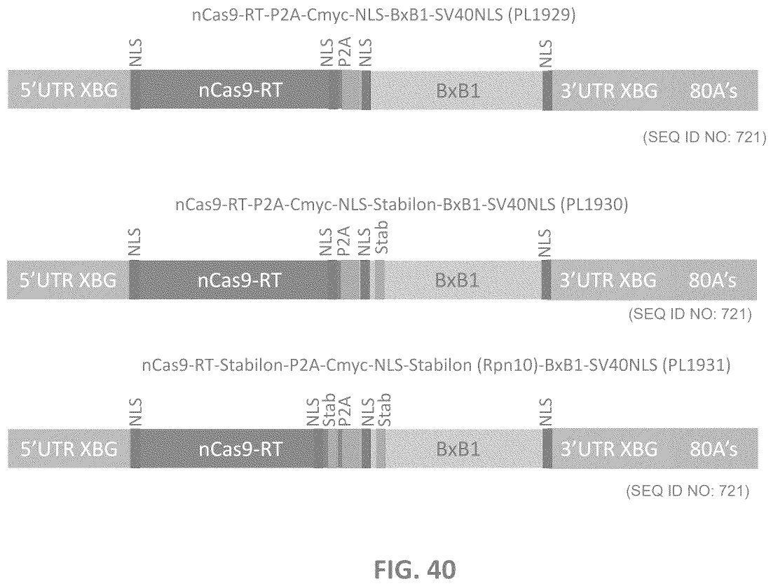

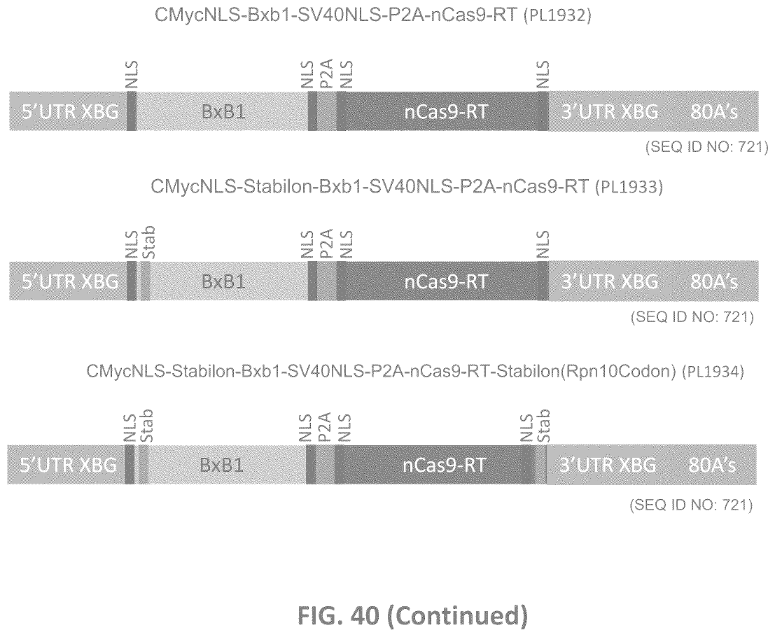

For these experiments, fusion proteins included those as shown in FIG. 40 (see also Table 26). These fusions proteins are introduced into cells along with an atgRNA and a donor polynucleotide template. Genomic DNA is harvested three days after transduction.

### 7.21. Example 21: Assessment of Split Fusion Proteins

Fusion proteins comprising a nCas9 and RT are fused with engineered integration enzymes (e.g., BxBT) in various combinations and tested for their ability to mediate PGI.

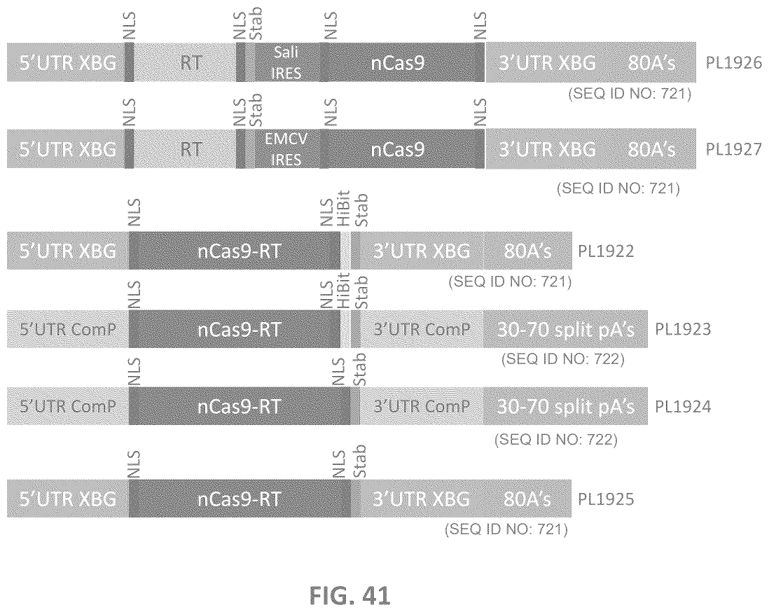

For these experiments, fusion proteins included those as shown in FIG. 41 (see also Table 26). These fusions proteins are introduced into cells along with an atgRNA and a donor polynucleotide template. Genomic DNA is harvested three days after transduction.

### 7.22. Example 22: Additional Assessment of Engineered Integration Enzyme in Induced Pluripotent Stem Cells

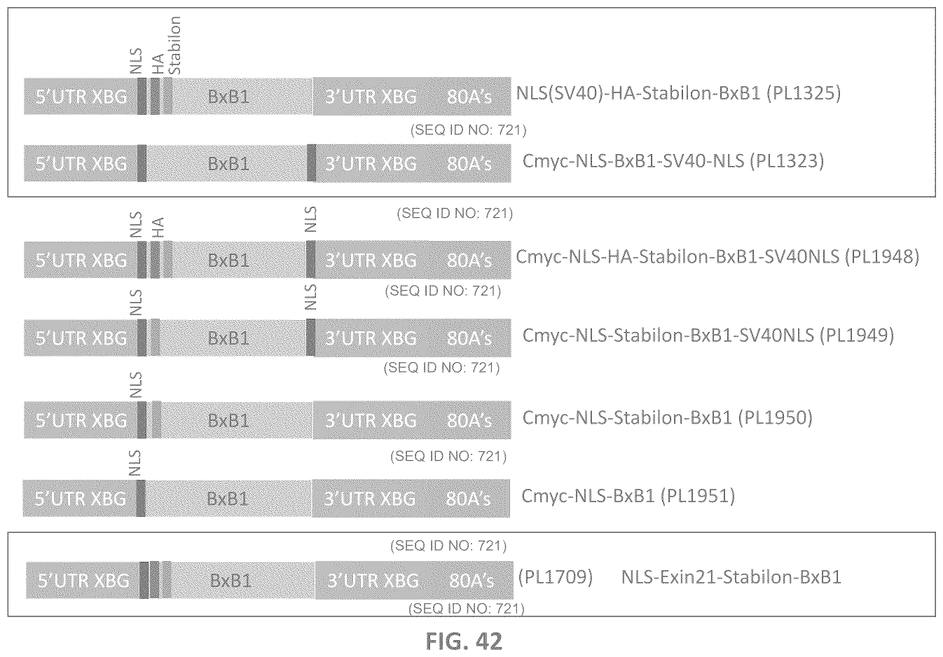

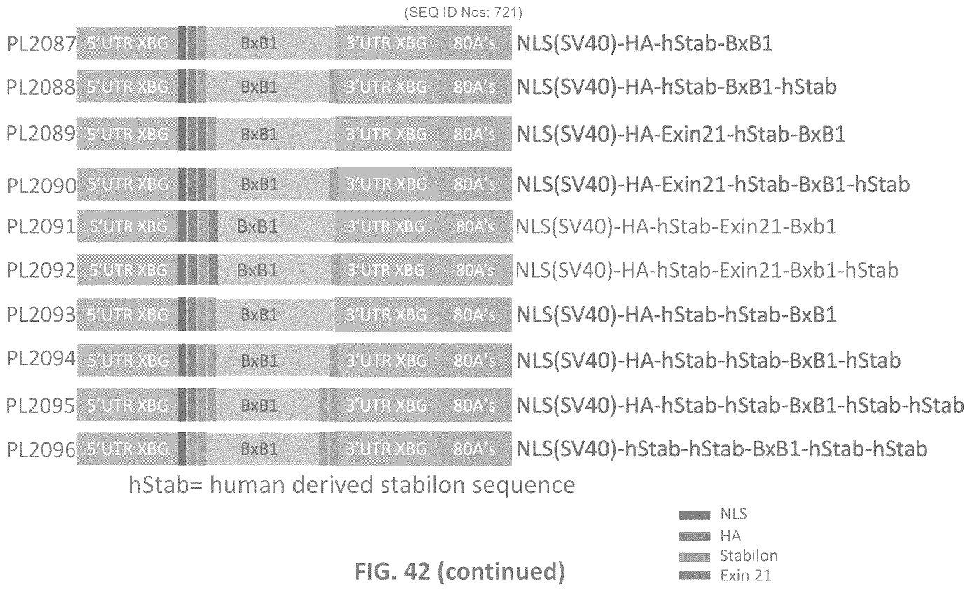

Engineered integration enzymes described and designed herein, including those in FIG. 42 (see also Table 26), were assessed for their ability to mediate PGI in pluripotent stem cells. A non-limiting exemplary workflow is as described in FIG. 30A.

For these experiments, the engineered integrases were introduced into a pluripotent stem cell clone 52 or clone 17 along and a donor polynucleotide template comprising a cognate integration recognition site (PL1113 (SEQ ID NO: 591; see Table 22) or PL2134. iPSC clones 52 and 17 include beacons site-specifically integrated into their genomes.

### Electroporation Conditions—

| | |
|---|---|
| Media | E8 media |
| Coating | Vitronectin |
| Cells/rxn | 1e5 cells |
| EP Program | 1200 V, 30 ms, 1 pulse |
| Recovery | E8 + CloneR2 24 h post-EP |
| Bxb1 mRNA | 2 ug/rxn |
| PL1113 [GT] Or PL2134 | 1 ug/rxn |

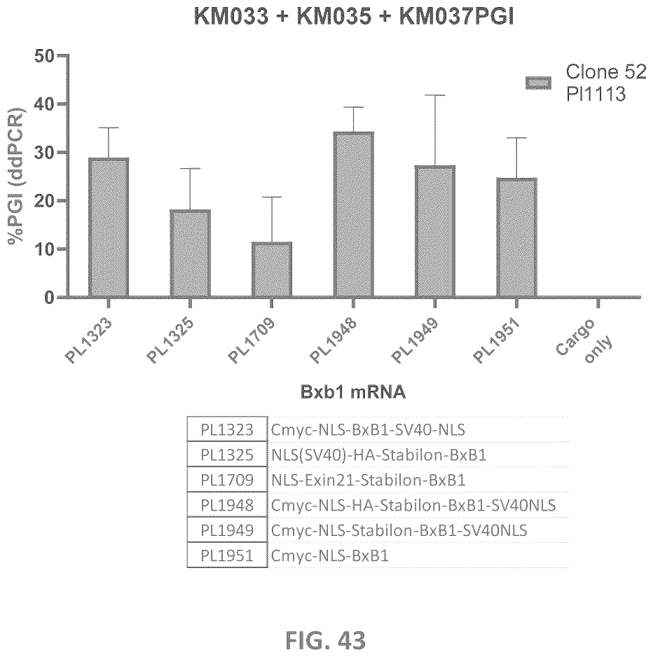

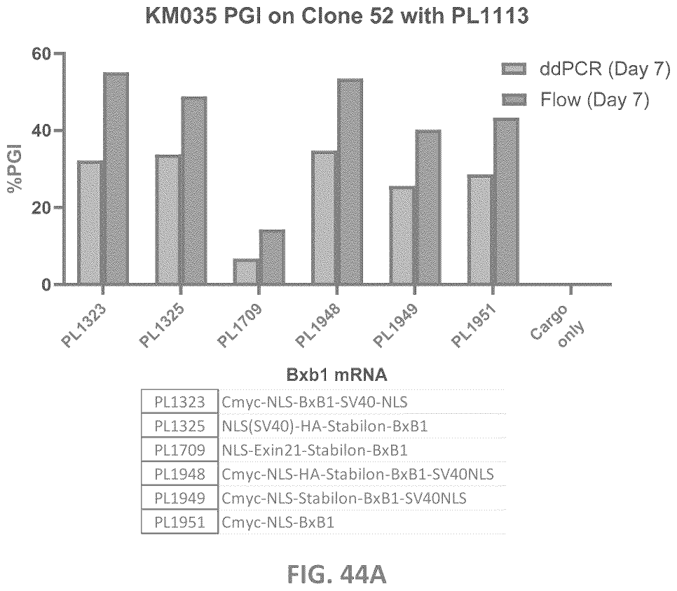

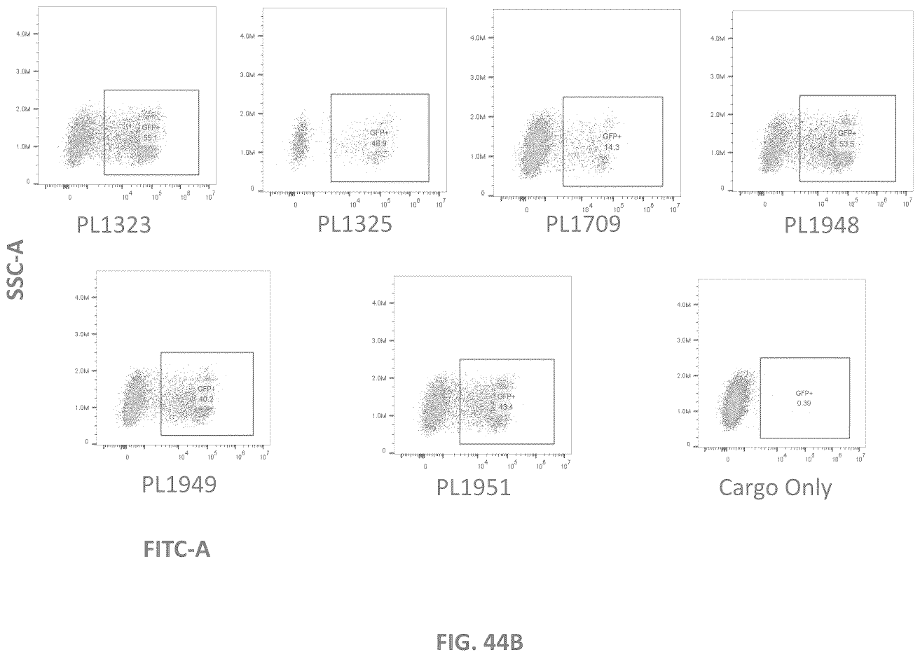

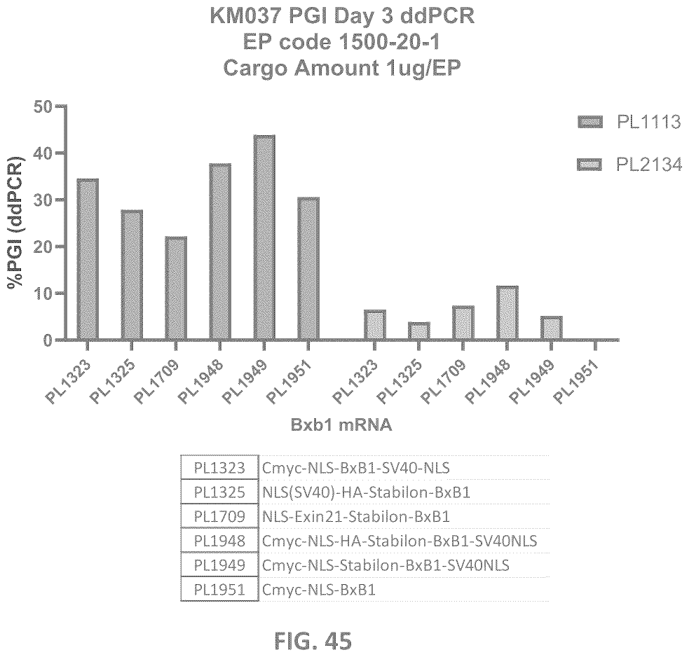

Genomic DNA was harvested 3 days after transduction. Digital droplet PCR (ddPCR) data for programmable gene insertion (PGI) with PL1113 donor at day 3 is shown in FIG. 43 for the indicated engineered BxBT polypeptides. Flow cytometry data for PGI with PL1113 donor at day 7 is shown in FIGS. 44A and 44B. FIG. 44A provides a comparison between percent PGI between ddPCR data and flow cytometry data. Flow cytometry was used to assess GFP expression, which indicates programmable gene insertion (PGI). As shown in FIG. 45, PGI with PL2134 donor was also assessed and compared to PGI with PL11113. This data shows that PGI was higher with PL11113 than PL2134.

### 7.23. Example 23: Assessment of Engineered Integration Enzymes Fused to a Gene Editor Polypeptide

Engineered integration enzymes fused to a gene editor polypeptide as described and designed herein, including those in FIG. 46 (see also Table 26), were assessed for their ability to mediate PGI. The engineered integration enzymes fused to a gene editor polypeptide are referred to herein as the all-in-one constructs.

In a first set of experiments, mRNA encoding the engineered integrases from FIG. 46 were introduced into a primary human hepatocytes (line HU8403) along with a pair of atgRNA (AA089: atgRNA1 is SEQ ID NO: 611 and atgRNA2 is SEQ ID NO: 612 (see Table 24)). Electroporation was performed as described elsewhere herein (see Example 22). mRNA was electroporated at either 187 fmol or 374 fmol. Controls included mRNA encoding nCas9-RT with and without a stabilization domain and a mRNA encoding RT-IRES-nCas9.

TABLE 24

| SEQ | | |
|---|---|---|
| ID | | |
| NO: | Name | Sequence |
| 611 | AA089 | CUUGUAUGCCCCGAGAAGUGGUUUUAGAGCUAGAAAUAGCAAGUUAAAAUAA |
| | atgRNA- | GGCUAGUCCGUUAUCAACUUGAAAAAGUGGCACCGAGUCGGUGCACGACGG |
| | modRNA478 | AGACCGCCGUCGUCGACAAGCCUUCUCGGGGCAUAUUUU |
| 728 | atg1 | m(C)[sp].m(U)[sp].m(U)[sp].r(G)p.r(U)p.r(A)p.r(U)p.r(G)p.r(C)p.r(C)p. |
| (mod) | (modRNA478) | r(C)p.r(C)p.r(G)p.r(A)p.r(G)p.r(A)p.r(A)p.r(G)p.r(U)p.r(G)p.r(G)p.r(U)p. |
| | RNA Helm | r(U)p.r(U)p.r(U)p.r(A)p.r(G)p.r(A)p.m(G)p.m(C)p.m(U)p.m(A)p.m(G)p.m(A)p. |
| | annotation | m(A)p.m(A)p.m(U)p.m(A)p.m(G)p.m(C)p.r(A)p.r(A)p.r(G)p.r(U)p.r(U)p.r(A)p. |
| | | r(A)p.r(A)p.r(A)p.r(U)p.r(A)p.r(A)p.r(G)p.r(G)p.r(C)p.r(U)p.r(A)p.r(G)p. |
| | | r(U)p.r(C)p.r(C)p.r(G)p.r(U)p.r(U)p.r(A)p.r(U)p.r(C)p.r(A)p.m(A)p.m(C)p. |
| | | m(U)p.m(U)p.m(G)p.m(A)p.m(A)p.m(A)p.m(A)p.m(A)p.m(G)p.m(U)p.m(G)p.m(G)p. |
| | | m(C)p.m(A)p.m(C)p.m(C)p.m(G)p.m(A)p.m(G)p.m(U)p.m(C)p.m(G)p.m(G)p.m(U)p. |
| | | m(G)p.m(C)p.r(A)p.r(C)p.r(G)p.r(A)p.r(C)p.r(G)p.r(G)p.r(A)p.r(G)p.r(A)p. |
| | | r(C)p.r(C)p.r(G)p.r(C)p.r(C)p.r(G)p.r(U)p.r(C)p.r(G)p.r(U)p.r(C)p.r(G)p. |
| | | r(A)p.r(C)p.r(A)p.r(A)p.r(G)p.r(C)p.r(C)p.r(U)p.r(U)p.r(C)p.r(U)p.r(C)p. |
| | | r(G)p.r(G)p.r(G)p.r(G)p.r(C)p.r(A)p.r(U)p.r(A)p.m(U)[sp].m(U)[sp]. |
| | | m(U)[sp].m(U)p |
| 612 | modRNA479 | UAUAUAUACUUGCUAGGGCUGUUUUAGAGCUAGAAAUAGCAAGUUAAAAUAA |
| | RNA | GGCUAGUCCGUUAUCAACUUGAAAAAGUGGCACCGAGUCGGUGCACGACGG |
| | | CGGUCUCCGUCGUCAGGAUCAUCCUAGCAAGUAUAUUUU |
| 729 | modRNA479 | m(U)[sp].m(A)[sp].m(U)[sp].r(A)p.r(U)p.r(A)p.r(U)p.r(A)p.r(C)p.r(U)p. |
| (mod) | Helm | r(U)p.r(G)p.r(C)p.r(U)p.r(A)p.r(G)p.r(G)p.r(G)p.r(C)p.r(U)p.r(G)p.r(U)p. |
| | | r(U)p.r(U)p.r(U)p.r(A)p.r(G)p.r(A)p.m(G)p.m(C)p.m(U)p.m(A)p.m(G)p.m(A)p. |
| | | m(A)p.m(A)p.m(U)p.m(A)p.m(G)p.m(C)p.r(A)p.r(A)p.r(G)p.r(U)p.r(U)p.r(A)p. |
| | | r(A)p.r(A)p.r(A)p.r(U)p.r(A)p.r(A)p.r(G)p.r(G)pr(C)p.r(U)p.r(A)p.r(G)p. |
| | | r(U)p.r(C)p.r(C)p.r(G)p.r(U)p.r(U)p.r(A)p.r(U)p.r(C)p.r(A)p.m(A)p.m(C)p. |
| | | m(U)p.m(U)p.m(G)p.m(A)p.m(A)p.m(A)p.m(A)p.m(A)p.m(G)p.m(U)p.m(G)p.m(G)p. |
| | | m(C)p.m(A)p.m(C)p.m(C)p.m(G)p.m(A)p.m(G)p.m(U)p.m(C)p.m(G)p.m(G)p.m(U)p. |
| | | m(G)p.m(C)p.r(A)p.r(C)p.r(G)p.r(A)p.r(C)p.r(G)p.r(G)p.r(C)p.r(G)p.r(G)p. |
| | | r(U)p.r(C)p.r(U)p.r(C)p.r(C)p.r(G)p.r(U)p.r(C)p.r(G)p.r(U)p.r(C)p.r(A)p. |
| | | r(G)p.r(G)p.r(A)p.r(U)p.r(C)p.r(A)p.r(U)p.r(C)p.r(C)p.r(U)p.r(A)p.r(G)p. |
| | | r(C)p.r(A)p.r(A)p.r(G)p.r(U)p.r(A)p.r(U)p.r(A)p.m(U)[sp].m(U)[sp]. |
| | | m(U)[sp].m(U)p |

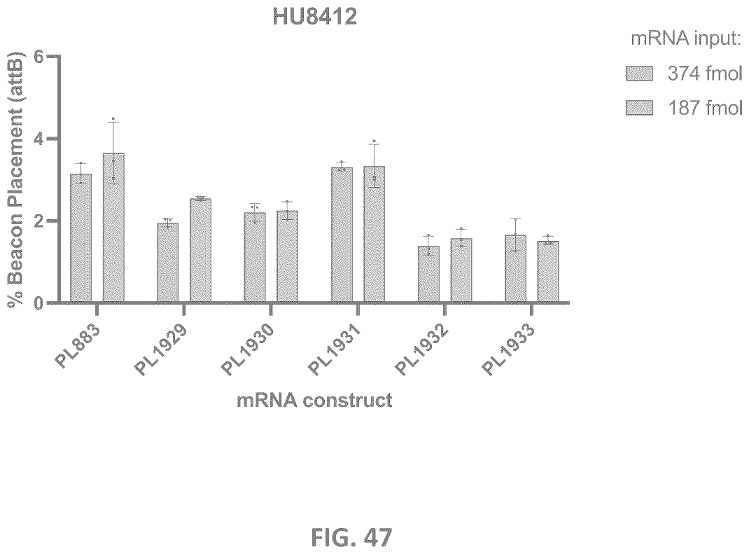

Genomic DNA was harvested 4 days after electroporation. Digital droplet PCR (ddPCR) was used to assess beacon placement for each of the constructs tested (see FIG. 46). As shown in FIG. 47, ddPCR data for beacon placement revealed for the all-in-one mRNAs were slightly lower than the control (PL883) but were comparable. Notably, the nCas9-RT-Bxb1 orientation produced more efficient beacon placement compared to the Bxb1-nCas9-RT orientation (see FIG. 47).

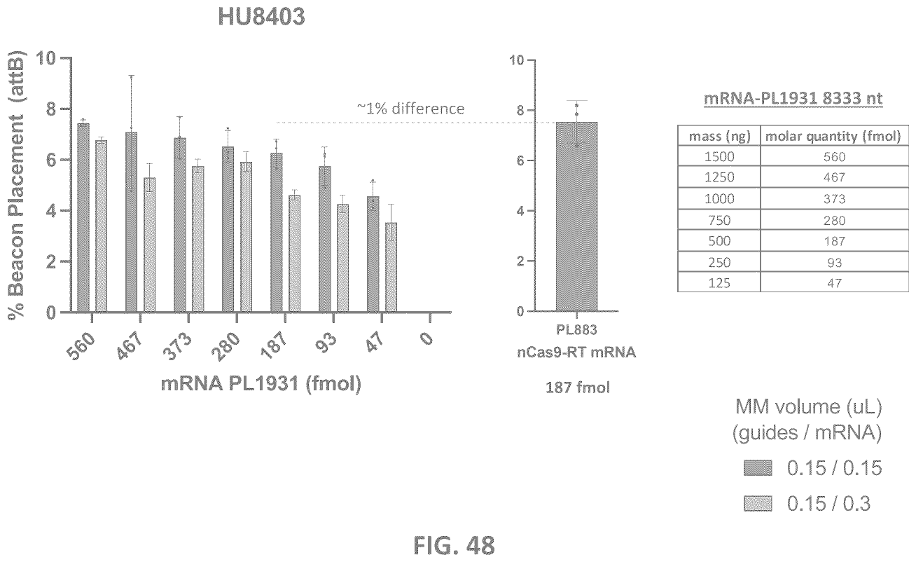

In a second set of experiments, mRNA encoding PL1931 ((SEQ ID NO: 631 (amino acid) and SEQ ID NO: 632 (nucleic acid); see FIG. 46) was introduced into a primary human hepatocyte (line HU8403) along with a pair of atgRNA (AA115 (atgRNA1 (SEQ ID NO: 613) and atgRNA2 (SEQ ID NO: 614); see Table below) using MessengerMax lipofection. At day 0, 33,000 HU8403 cells were plated per well and MessengerMax, 0.15 μl atgRNA pair, and either 0.15 μl or 0.3 d of various concentrations of Bxb1 mRNA (see FIG. 48). PL883 (SEQ ID NO: 617) was used as a control. Genomic DNA was harvested 4 days after lipofection. Digital droplet PCR (ddPCR) was used to assess beacon placement. As shown in FIG. 48, beacon placement was similar between mRNA PL1931 and mRNA PL883 at equimolar input.

TABLE 25

| SEQ | | |
|---|---|---|
| ID | | |
| NO: | Name | Sequence |
| 613 | atgRNA1 | UUGGCUUCUAAAAGGAGUUUGUUUUAGAGCUAGAAAUAGCAAGUUA |
| | modRNA519 | AAAUAAGGCUAGUCCGUUAUCAACUUGAAAAAGUGGCACCGAGUCGG |
| | RNA | UGCACGACGGAGACCGCCGUCGUCGACAAGCCCUCCUUUUAGAAG |
| 730 | atgRNA1 | m(U)[sp].m(U)[sp].m(G)[sp].r(G)p.r(C)p.r(U)p.r(U)p.r(C)p.r(U)p.r(A)p.r(A)p. |
| (mod) | modRNA519 | r(A)p.r(A)p.r(G)p.r(G)p.r(A)p.r(G)p.r(U)p.r(U)p.r(U)p.r(G)p.r(U)p.r(U)p. |
| | Helm | r(U)p.r(U)p.r(A)p.r(G)p.r(A)p.r(G)p.r(C)p.r(U)p.r(A)p.r(G)p.r(A)p.r(A)p. |
| | | r(A)p.r(U)p.r(A)p.r(G)p.r(C)p.r(A)p.r(A)p.r(G)p.r(U)p.r(U)p.r(A)p.r(A)p. |
| | | r(A)p.r(A)p.r(U)p.r(A)p.r(A)p.r(G)p.r(G)p.r(C)p.r(U)p.r(A)p.r(G)p.r(U)p. |
| | | r(C)p.r(C)p.r(G)p.r(U)p.r(U)p.r(A)p.r(U)p.r(C)p.r(A)p.r(A)p.r(C)p.r(U)p. |
| | | r(U)p.r(G)p.r(A)p.r(A)p.r(A)p.r(A)p.r(A)p.r(G)p.r(U)p.r(G)p.r(G)p.r(C)p. |
| | | r(A)p.r(C)p.r(C)p.r(G)p.r(A)p.r(G)p.r(U)p.r(C)p.r(G)p.r(G)p.r(U)p.r(G)p. |
| | | r(C)p.r(A)p.r(C)p.r(G)p.r(A)p.r(C)p.r(G)p.r(G)p.r(A)p.r(G)p.r(A)p.r(C)p. |
| | | r(C)p.r(G)p.r(C)p.r(C)p.r(G)p.r(U)p.r(C)p.r(G)p.r(U)p.r(C)p.r(G)p.r(A)p. |
| | | r(C)p.r(A)p.r(A)p.r(G)p.r(C)p.r(C)p.r(C)p.r(U)p.r(C)p.r(C)p.r(U)p.r(U)p. |
| | | r(U)p.r(U)p.r(A)p.r(G)[sp].m(A)[sp].m(A)[sp].m(G) |
| 614 | atgRNA2 | ACUGUUGCAAUAAUACCUAAGUUUUAGAGCUAGAAAUAGCAAGUUAAAAUAA |
| | modRNA566 | GGCUAGUCCGUUAUCAACUUGAAAAAGUGGCACCGAGUCGGUGCACGACGG |
| | RNA | CGGUCUCCGUCGUCAGGAUCAUGGUAUUAUUGCAA |
| s731 | atgRNA2 | m(A)[sp].m(C)[sp].m(U)[sp].r(G)p.r(U)p.r(U)p.r(G)p.r(C)p.r(A)p.r(A)p.r(U)p. |
| (mod) | modRNA566 | r(A)p.r(A)p.r(U)p.r(A)p.r(C)p.r(C)p.r(U)p.r(A)p.r(A)p.r(G)p.r(U)p.r(U)p. |
| | Helm | r(U)p.r(U)p.r(A)p.r(G)p.r(A)p.r(G)p.r(C)p.r(U)p.r(A)p.r(G)p.r(A)p.r(A)p. |
| | | r(A)p.r(U)p.r(A)p.r(G)p.r(C)p.r(A)p.r(A)p.r(G)p.r(U)p.r(U)p.r(A)p.r(A)p. |
| | | r(A)p.r(A)p.r(U)p.r(A)p.r(A)p.r(G)p.r(G)p.r(C)p.r(U)p.r(A)p.r(G)p.r(U)p. |
| | | r(C)p.r(C)p.r(G)p.r(U)p.r(U)p.r(A)p.r(U)p.r(C)p.r(A)p.r(A)p.r(C)p.r(U)p. |
| | | r(U)p.r(G)p.r(A)p.r(A)p.r(A)p.r(A)p.r(A)p.r(G)p.r(U)p.r(G)p.r(G)p.r(C)p. |
| | | r(A)p.r(C)p.r(C)p.r(G)p.r(A)p.r(G)p.r(U)p.r(C)p.r(G)p.r(G)p.r(U)p.r(G)p. |
| | | r(C)p.r(A)p.r(C)p.r(G)p.r(A)p.r(C)p.r(G)p.r(G)p.r(C)p.r(G)p.r(G)p.r(U)p. |
| | | r(C)p.r(U)p.r(C)p.r(C)p.r(G)p.r(U)p.r(C)p.r(G)p.r(U)p.r(C)p.r(A)p.r(G)p. |
| | | r(G)p.r(A)p.r(U)p.r(C)p.r(A)p.r(U)p.r(G)p.r(G)p.r(U)p.r(A)p.r(U)p.r(U)p. |
| | | r(A)p.r(U)p.r(U)p.r(G)[sp].m(C)[sp].m(A)[sp].m(A) |

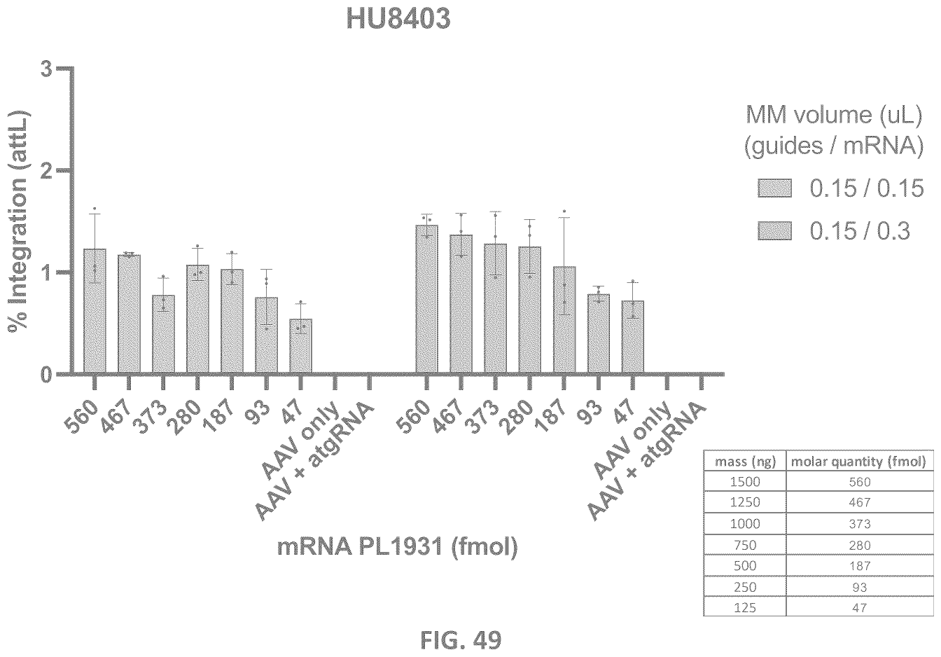

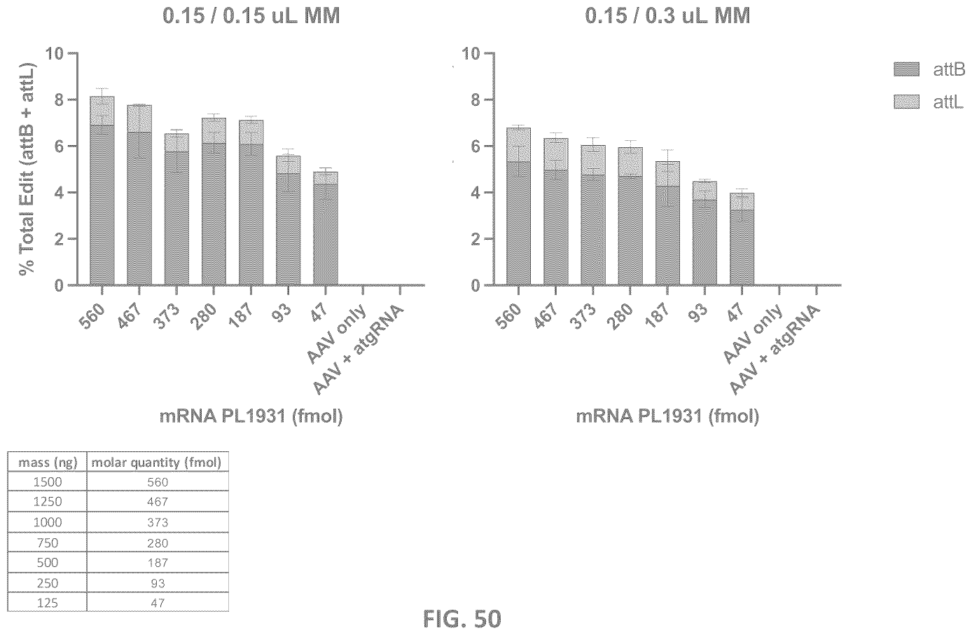

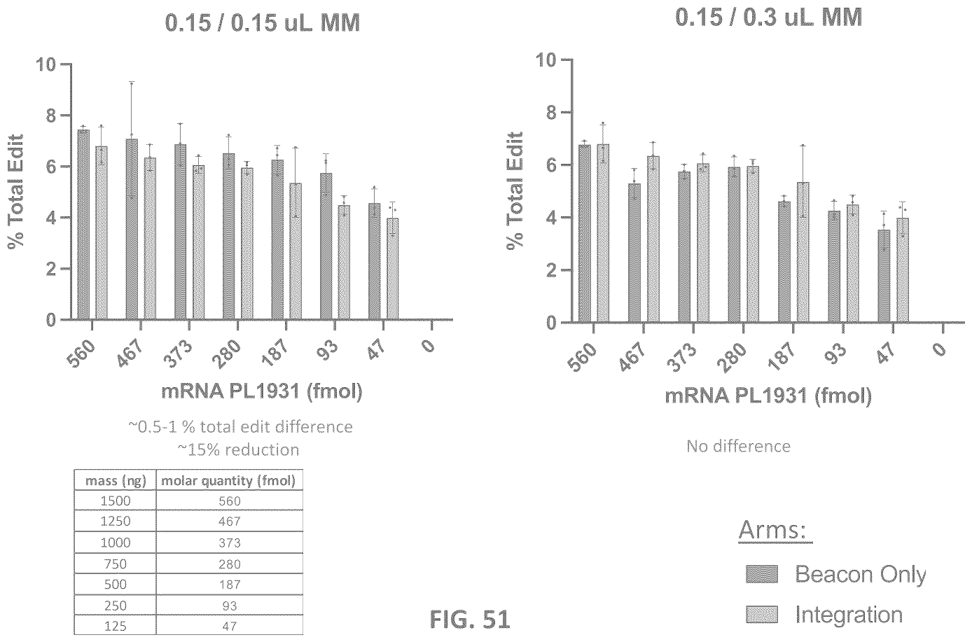

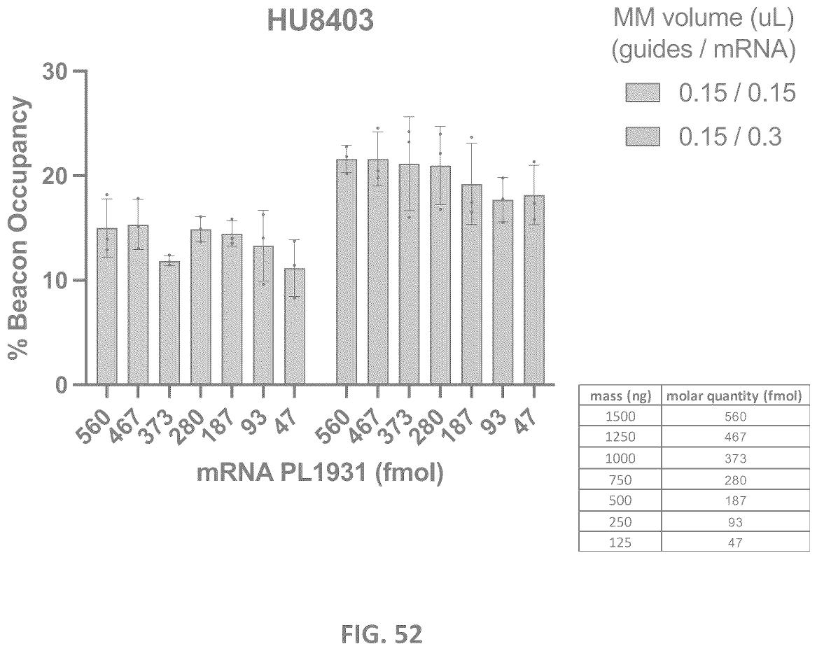

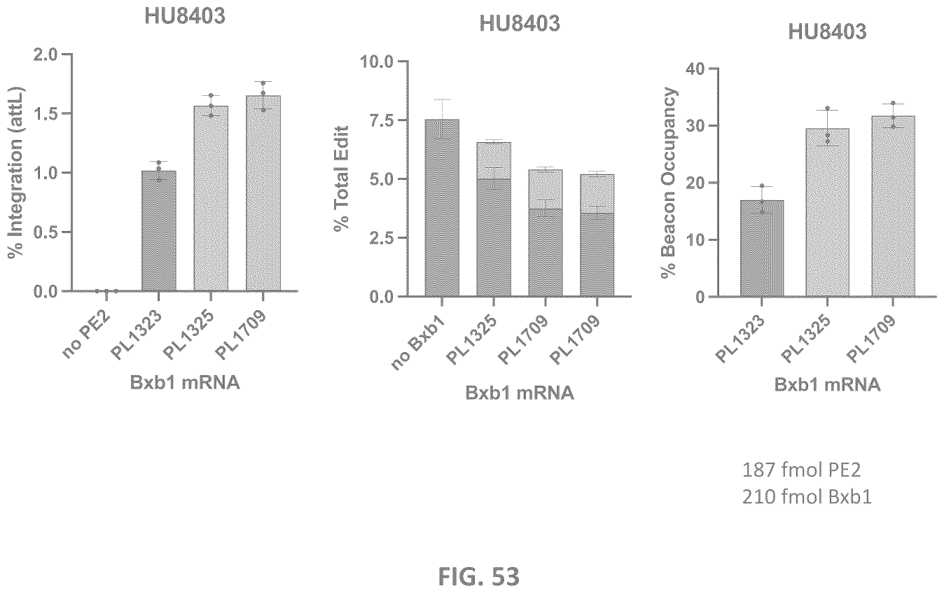

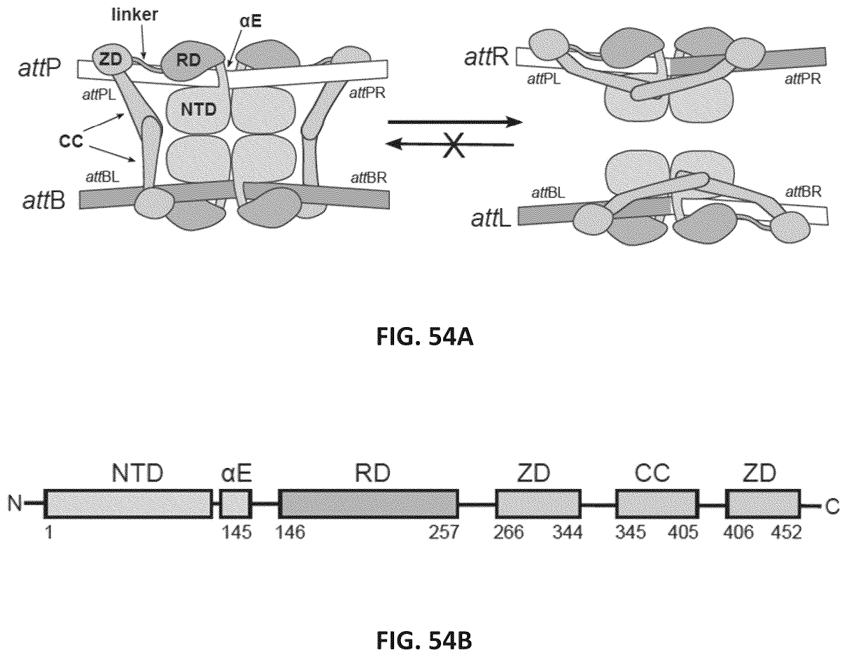

In a third set of experiments, PGI in PHH was compared following lipofection with an mRNA encoding PL1931 (“all-in-one”) (SEQ ID NO: 631 (amino acid) and SEQ ID NO: 632 (nucleic acid)); see FIG. 46) or two mRNAs one encoding nCas9-RT and a second encoding BxB1. For these experiments, mRNA was introduced into PHH line HU8403 along with a pair of atgRNA (AA115) using MessengerMax lipofection. In particular, for the PL1931 all-in-one, 0.15 μl of atgRNA pair, and either 0.15 μl or 0.3 μl of various concentrations of Bxb1 mRNA were transduced into the PHH (see FIG. 49 for concentrations of BxB1). For the two mRNA condition (i.e., separate mRNA encoding nCas9-RT and BxB1), BxB1 was introduced at 210 fmol (or ˜125 ng) (note specific constructs included: PL1323 (cmyc-NLS-no stabilon), PL1325 (SV40-NLS-Stabilon), and PL1709 (SV40-exin2l-stabilon). For some conditions, a 3 lipoplex approach was used: guides 0.15 μl per well (20 mM) with 0.15 μl MessengerMax (or 30 μl MessengerMax); BxB1 mRNA at 250 ng with 0.15p MessengerMax (or 30 μl MessengerMax) per well; and nCas9-RT mRNA PL883 (SEQ ID NO: 617) fixed at 400 ng or 187 fmol with 0.15 μl MessemgerMax (or 30 μl MessengerMax) per well. Genomic DNA was harvested 4 days after lipofection. Digital droplet PCR (ddPCR) was used to assess PGI. As shown in FIG. 49, PGI was around 1% for each of the conditions tested. Looking closer at each PGI event, FIG. 50 and FIG. 51 show data for total edits (AttB+AttL). Analysis of whether MessengerMax volume impacted beacon placement revealed that increased MessengerMax volume decreased beacon placement but increased integration (FIG. 52). Analysis of the 3 lioplex approach in FIG. 53, revealed a 20-30% hit to beacon placement ratio observed with integration samples (3 lipo) compared to beacon only samples (2 lipo). This data shows that mRNAs encoding a BxB1-stabilon result in increased beacon occupancy compared to mRNA encoding BxB1 without a stabilon (see PL1323 (SEQ ID NO: 618 (amino acid) and SEQ ID NO: 619 (nucleic acid)).

Taken together, these experiments show that PL1931 all-in-one achieves full PGI in PHH; modulating MessengerMax volume decreases overall beacon placement but does increase integration and subsequent occupancy with the 2 lipoplex approach; and integration efficiency is similar from one shot transfections of all-in-one mRNA PL1931 (2 lipoplex) versus the two mRNA (separate nCas9-RT and BxB1).

### 7.24. Example 24: Mutations in the Zinc Ribbon Domain (ZD) Maintain Integration Activity while Improving Specificity (e.g., Reduced Off-Target Integration)

This experiment demonstrates that certain mutations in the zinc ribbon domain of LSR can improve specificity of such integrases to reduce or eliminate off-target integration.

Briefly, partly based on structural information, about 22 single residue mutations of the Bxb1 integrase were generated, at ZD domain residues 315, 316, 317, 318, 319, and 320. At each position, wild-type residue was mutated to a different category of amino acids. For example, A315 was mutated to acidic residues D and E, as well as basic residue R. G316, G317 and G318 were each independently mutated to polar residue Q, acidic residue E, basic residue R, and aromatic residue W. R319 was mutated to acidic residue E, polar residue Q, and aromatic residue W. K320 was mutated to polar residue Q, acidic residue E, basic residue R, and aromatic residue W. These mutants were introduced into a test mammalian cell line HEK293, in which integrase activity at the cognate integration recognition sequences attB/attP, or at non-cognate off-site integration recognition sequences (such as CAS031 or CAS 421, see FIG. 59) can be assessed based on, for example, the ability or efficiency of the integrase/eLSR to put a promoter sequence upstream of an otherwise promoterless reporter gene (such as a luciferase coding sequence), depending on the efficiency of using the cognate integration recognition sequences attB/attP, or the non-cognate off-site integration recognition sequences (such as CAS031 or CAS 421, see FIG. 59).

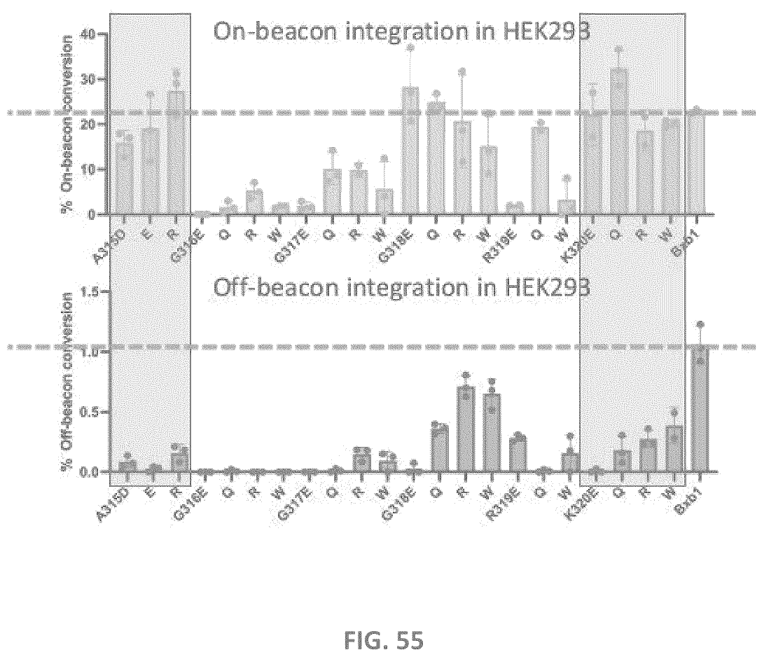

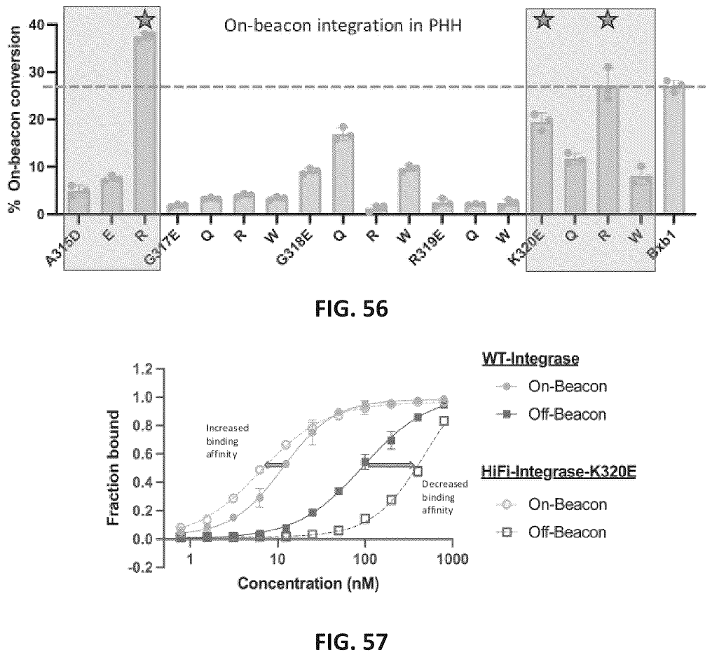

The results of such assays conducted in HEK293 cells showed that mutating residues A315 or K320 to a number of different amino acids can maintain on-beacon activity while decrease off-beacon activity as compared to wild type Bxb1 (FIGS. 55A and 55B). Similar results were observed in Primary Human Hepatocytes (PHH) (FIG. 56), in which mutations at A315 to R and K320 to E or R did not substantially impact on-beacon integration activity.

Among the tested mutations, K320E was one of the lead candidates with on-beacon activity comparable to wild type integrase and undetectable off-beacon activity (FIG. 55B). This mutation of K320E also led to a ˜2-fold increase in affinity for on-beacon sites and a 2-˜5-fold decrease in affinity for off-beacon sites (FIG. 57).

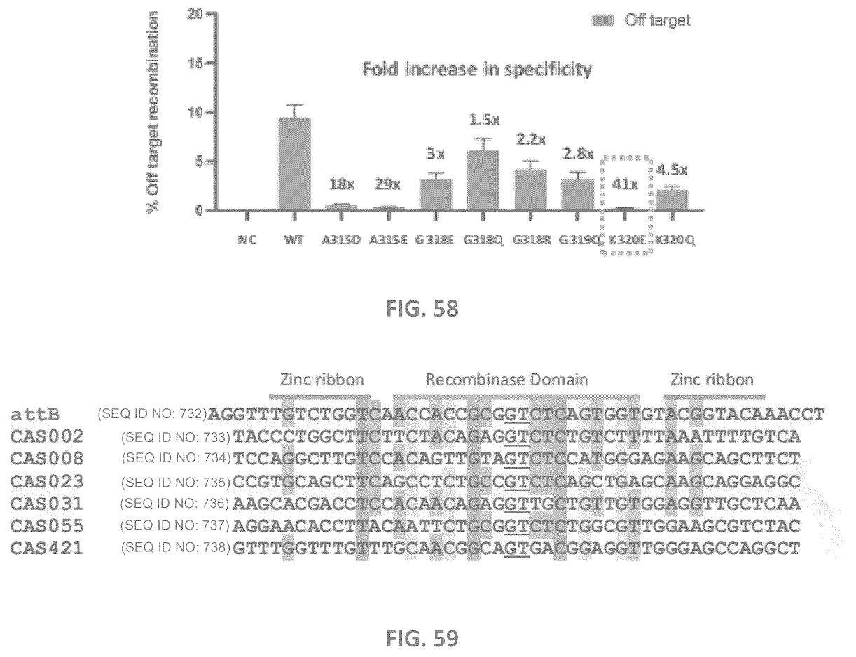

Consistent with the results discussed above, 8 of the selected eLSRs displayed reduced frequency and scope of off-target edits at 100 top off-target sites. All such so-called HiFi variant eLSRs exhibited improved off target integration percentage (%) vs wild type Bxb1 integrase, from which the eLSRs derived. Remarkably, the K320E single mutant decreased off-target recombination % by 41-fold as compared to that of wild type Bxb1 integrase (FIG. 58).

### 7.25. Example 25: Engineered Bxb1 Variants Improve Integrase Activity and Fidelity

### 7.25.1. Abstract

Many current genome editing technologies rely on the action of large serine integrases (LSIs) to insert gene-sized DNA sequences into the genome. Bxb1 is the most commonly used LSI for therapeutic efforts, including PASTE, PASSIGE and I-PGI. While Bxb1 demonstrated good activity in vitro in cycling cells, the activity in non-dividing hepatocytes was significantly less efficient. Further, wild-type Bxb1 is known to have detectable off target activity at cryptic genomic sites, which presents a potential safety risk for therapeutic development. To address these issues, we developed Bxb1 variants that demonstrate increased specificity and potency in vitro and have engineered stabilized Bxb1 variants that increase in vivo activity over 25-fold enabling targeted integration at therapeutically relevant levels.

### 7.25.2. Introduction

Large serine integrases (LSIs) can insert any size and sequence of DNA into a specific location, given the presence of two complimentary binding sites for the LSI (1). This has inspired both gene therapy applications as well as cell line creation technologies (2-9). All LSIs contain four major structural domains, two of which, the recombinase and zinc ribbon, are responsible for recognizing the LSI's DNA binding sites (10). Following binding of the DNA substrates (attB and attP), LSIs undergo a structural rearrangement and create a new DNA topology resulting in the insertion of DNA (11).

For many applications, the LSIs that seem to be the most studied are Bxb1 and PhiC31 (12). Bxb1 appears to be more active overall and has emerged as the enzyme of choice for large gene insertion therapies (6,8,9,13). Bxb1 can insert large DNA sequences without relying on any endogenous cellular DNA repair machinery (14). The cargo is delivered as a fully synthesized gene without need for DNA repair as the insertion is done seamlessly between two short attachment site sequences (10,11).

However, Bxb1 has two previously characterized limitations: specificity (off-targets) and low activity in certain cell types (6,7,13,15). Bxb1 can use imperfect attachment sites (cryptic sites) as locations for integration, several of which are present in the human genome (7,9,15). Wild-type Bxb1 also has relatively modest activity in cells (8,16). This makes it impractical to use for gene insertion as most cures rely on a minimum amount of active protein expression to be viable.

Here we describe a set of engineered and naturally occurring Bxb1 variants that overcome these limitations. These variants introduce mutations that lead to highly specific integrases and stabilization tags that increase in vivo activity. We anticipate that these enzymes will be of significant use in further efforts for large gene insertion for therapeutic benefit.

### 7.25.3. Results

### 7.25.3.1 Bxb1 is a Highly Active, Site-Specific Recombinase

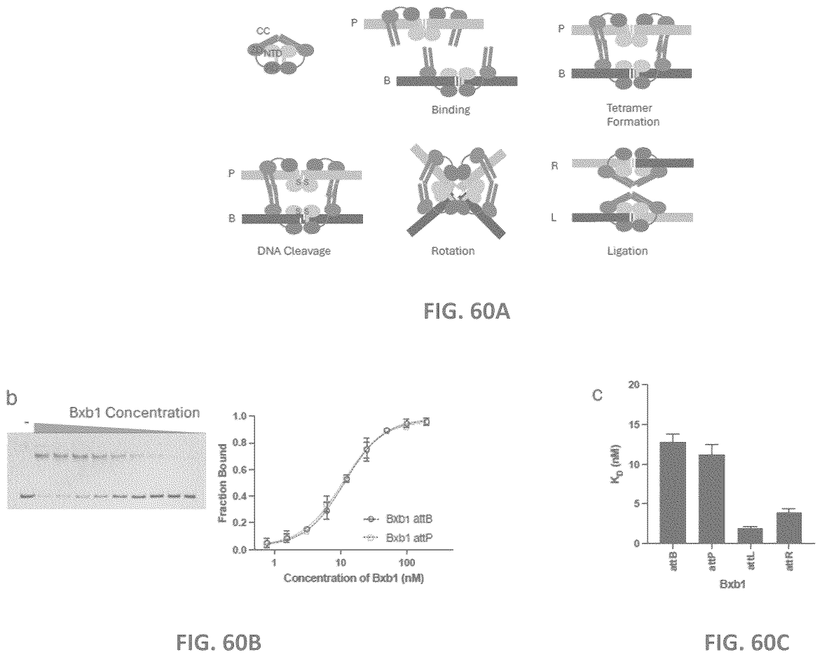

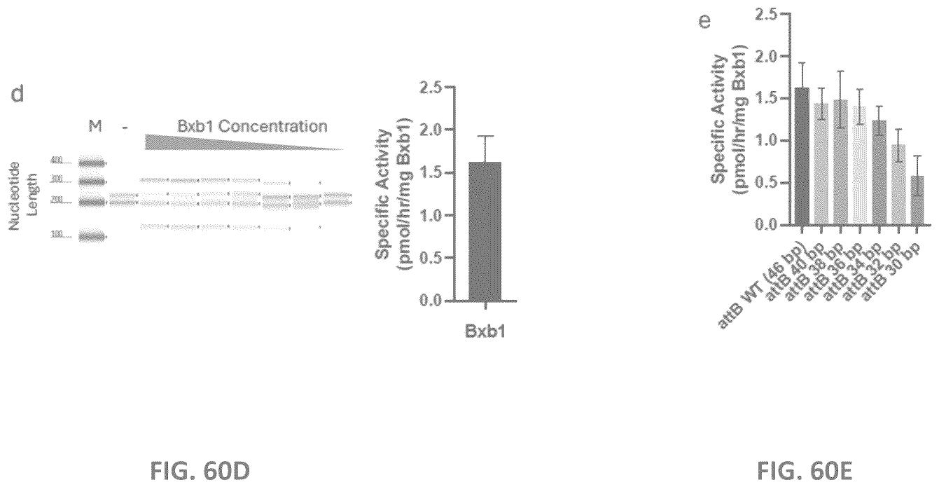

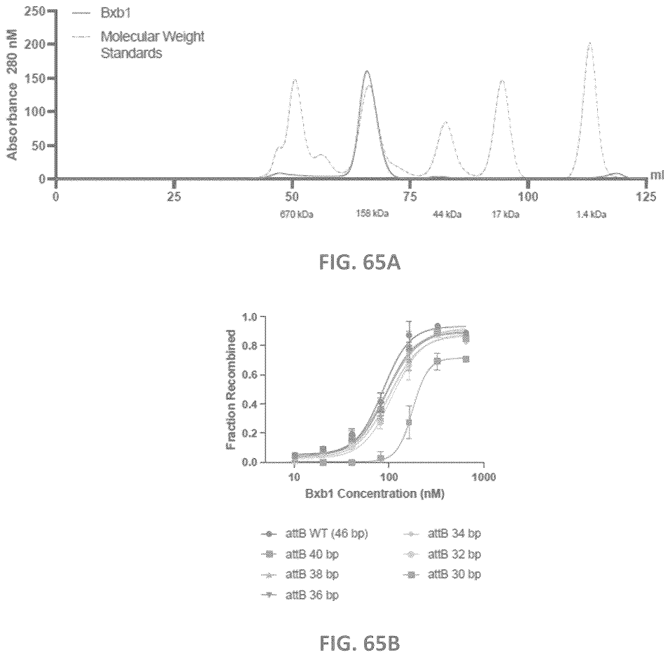

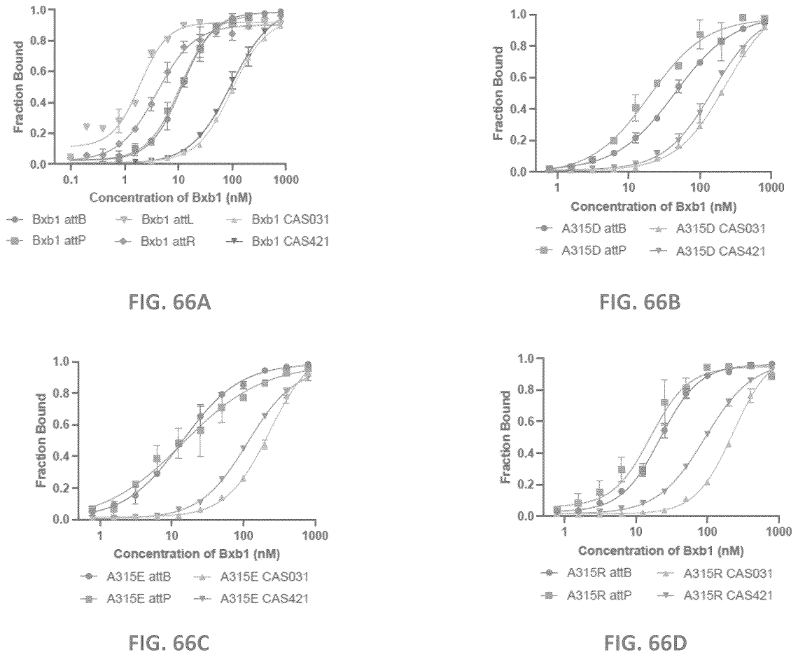

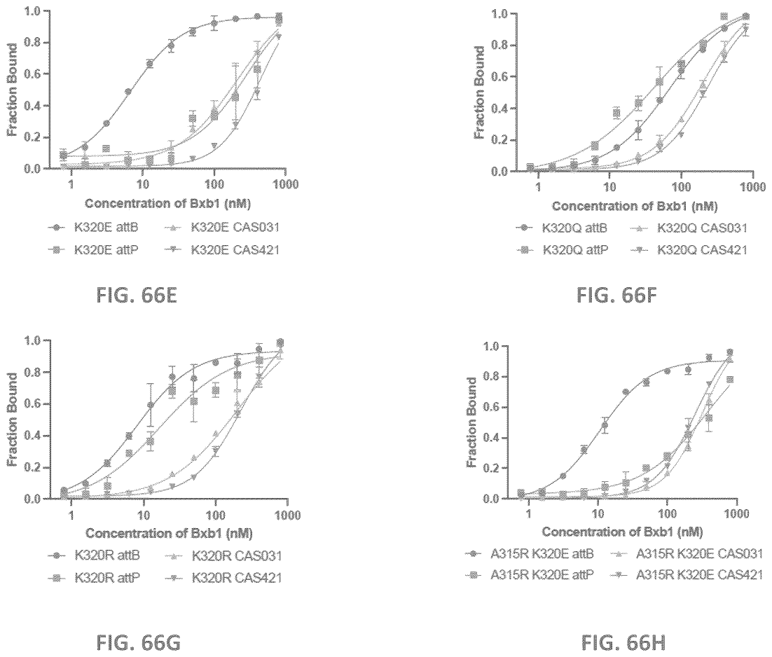

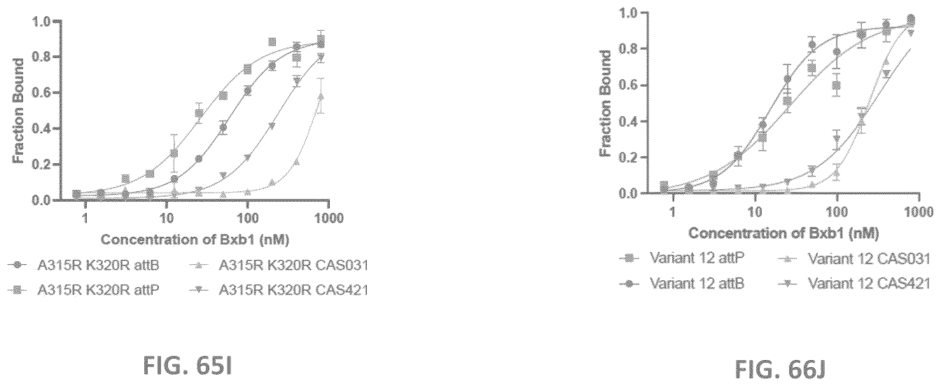

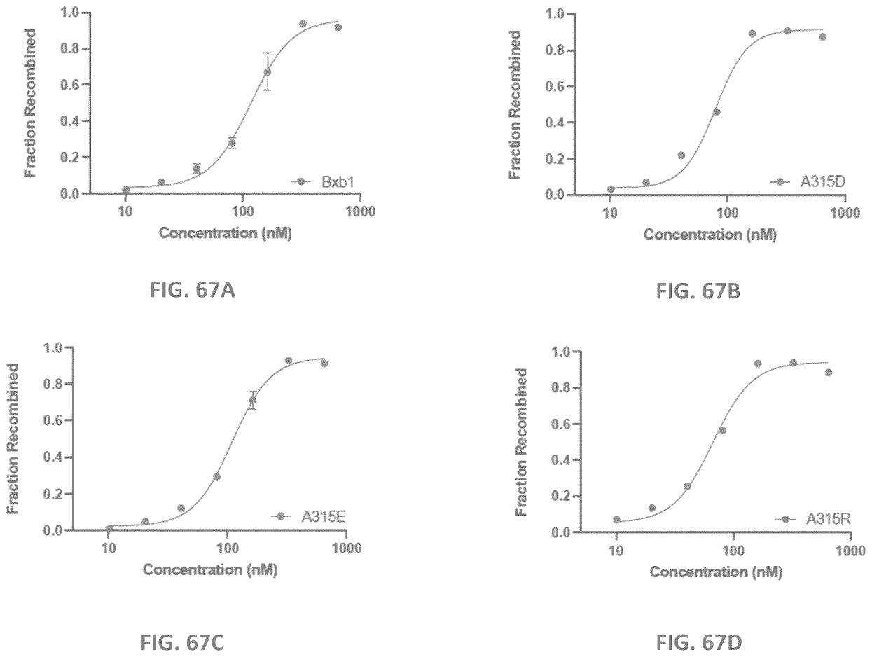

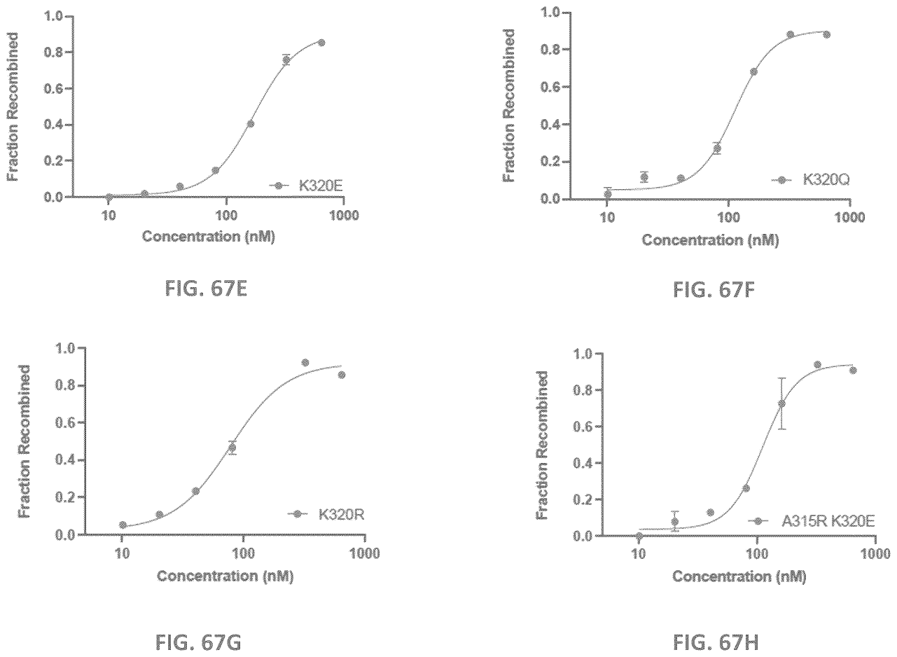

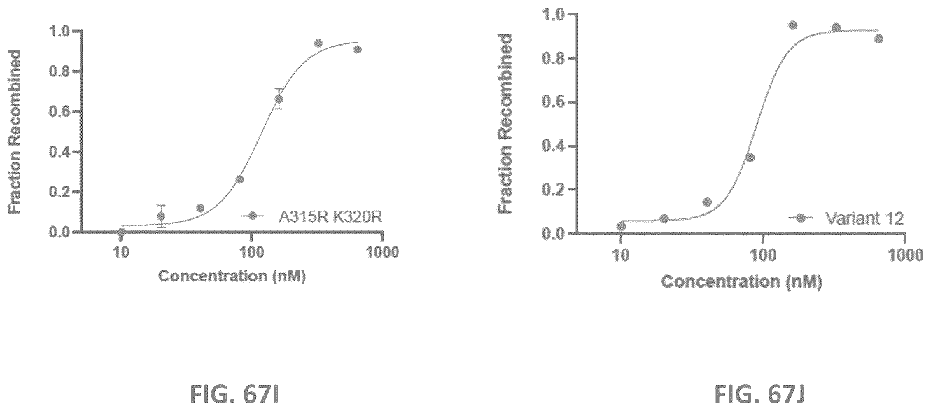

Four molecules of a large serine integrase (LSI) bind two DNA substrates, attB and attP, and catalyze a topological rearrangement leading to two DNA products, attL and attR (FIG. 60A) (11). LSIs have a conserved structural organization: the N-terminal domain contains a dimerization domain and the catalytic serine, two domains for DNA binding, a recombinase domain, and a zinc ribbon domain and a coiled-coil domain that mediate tetramer formation between two LSI dimers (1). For Bxb1, the DNA sequences of the substrates and products are well-defined and contain known binding sites for the recombinase and zinc ribbon domains (17). In the absence of DNA, Bxb1 is a homodimer (FIG. 65A). Using gel shift assays with fluorescently labeled substrates, we can accurately measure the affinity of Bxb1 for DNA substrates (FIG. 60B). Bxb1 dimers bind with relatively high affinity to both the substrate and product with Bxb1 dimers binding to the product sequences, attL and attR, with affinities of around 3 nM, and to the substrate sequences, attB and attP, with slightly weaker affinity of around 12 nM (FIG. 60C). At high concentrations, slow migrating species forms, suggestive of non-productive multimerization, but there is no evidence for DNA rearrangement without both substrates being present.

In its native environment, Mycobacterium smegmatis, the Mycobacteriophage protein Bxb1 catalyzes the integration of its own phage genome into the genome of the mycobacterium (17). At a biochemical level, a Bxb1 dimer sits on either of the DNA substrates (either attP or attB) and then forms a tetramer bringing the two substrates into proximity (18). A serine in the active site of Bxb1 attacks the phosphodiester backbone creating a covalent phosphoserine linkage between all strands of the DNA and rotation of the two half-sites leads to the ligation of the DNA backbones and formation of attL and attR (18,19). Using a gel-based assay and substrates of varying length we can measure the specific activity of LSI's. Under saturating substrate conditions, Bxb1 can catalyze this reaction with a specific activity of 1.5 pmol/hr/mg Bxb1 (FIG. 60D). Further, in the absence of an accessory protein, Bxb1, like other LSIs, only catalyzes the forward reaction (20).

The DNA sequence of the substrates for Bxb1 has been well defined but the minimal attB sequence required for full recombination activity was unknown (17). We created a series of attB substrates that were all the same length (180 bp) but had the sequence of the attB mutated from either end. By measuring the activity of each of the substrates we were able to demonstrate that a 34-base-pair attB leads to a similar specific activity as the full-length, 46-base-pair attB (FIG. 60E).

### 7.25.3.2 Mutations in the Zinc Ribbon Domain Affect Bxb1 Activity in HEK293FT Cells

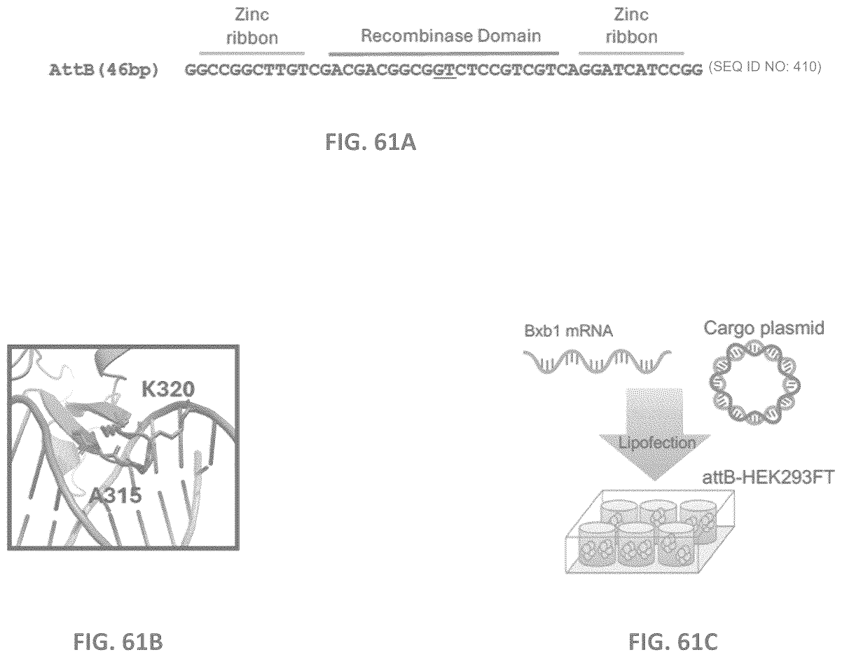

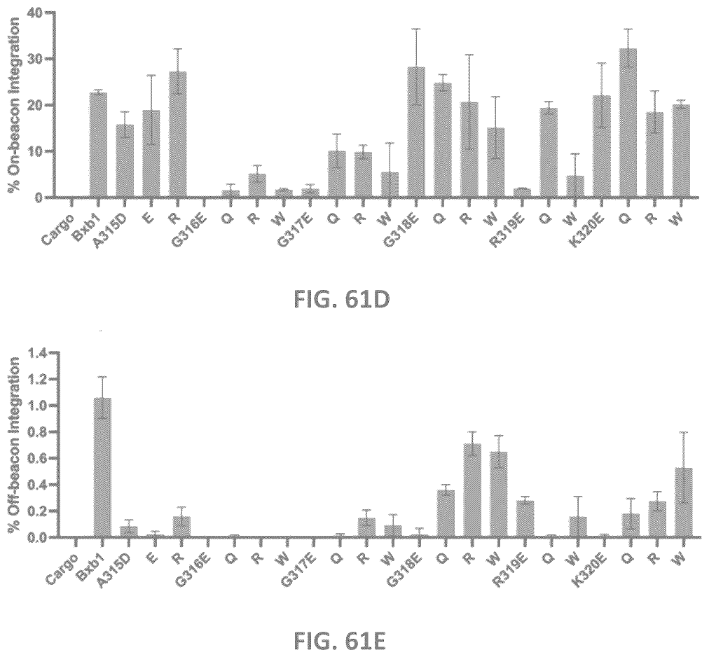

The zinc ribbon domain of Bxb1 is a compact DNA binding structure that is formed from two disparate regions in the linear sequence of Bxb1, with the coiled-coil domain inserted in the middle (FIG. 61A) (10,11,21). This organization is highly conserved among LSIs. Using the published structure of the Listeria innocua integrase, we designed a series of point mutants within the zinc ribbon domain to probe the interaction between Bxb1 and its substrates (10,21). These mutations were focused on a series of alanine and glycine residues in a loop within the zinc ribbon domain (FIG. 61B).

Mutation in the zinc ribbon domain had variable effects on integration of a DNA cargo by Bxb1. Following transfection of cargo DNA and Bxb1 mRNA into HEK293FT cells with a preplaced attB (here called ‘beacon’), we probed for on-beacon integration using ddPCR (FIG. 61C). Sub-saturating levels of both the DNA cargo and Bxb1 mRNA were used to highlight any differences amongst these Bxb1 variants. Under these conditions, we observed about 20% integration of a DNA cargo with a wild-type Bxb1 construct (FIG. 61D). We assessed the impact of mutations in the zinc ribbon domain to on-beacon integration by engineered Bxbis using the same assay. At certain positions, like A315 and R319, we saw reduced activity for some mutants but retained (or improved) activity for other mutations (FIG. 61D). Some positions, including G316 and G317, did not seem to be amenable to mutation or at least not for the amino acids we tested. Finally, at positions G318 and K320, we found that a range of mutations was tolerated.

Comprehensive cataloguing of Bxb1 substrate preferences has identified a number of cryptic sites in the human genome that Bxb1 can use as a site for integration (15). Many of the sites results in very low levels of integration but could still be problematic when using Bxb1 in a therapeutic context (7,9,15). In HEK293, the most prominent site of misintegration is a site on chromosome 6, CAS031 (cryptic attachment site 31) (15). We probed the extent to which zinc ribbon Bxb1 mutants can integrate at this site to identify an enzyme that has high on-beacon and reduced off-beacon integration (FIG. 61E).

Again, mutation in the zinc ribbon domain of Bxb1 had a variable effect on integration into this off-beacon location. Probing for integration at CAS031 using wild-type Bxb1 leads to about 1% integration under these experimental conditions (FIG. 61E). All the engineered Bxb1 constructs were less active on CAS031 than wild-type Bxb1, though some still retained significant activity. However, mutations at A315 appeared to significantly reduce off-beacon activity at CAS031 while retaining on-beacon integration. This is also true for G318 and K320 to glutamic acid and R319 to glutamine.

### 7.25.3.3 Biochemical Characterization of Zinc Ribbon Domain Mutants

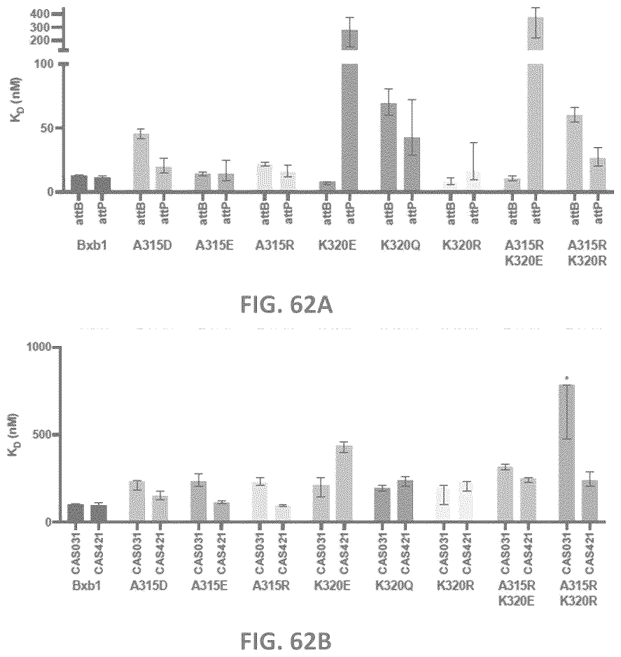

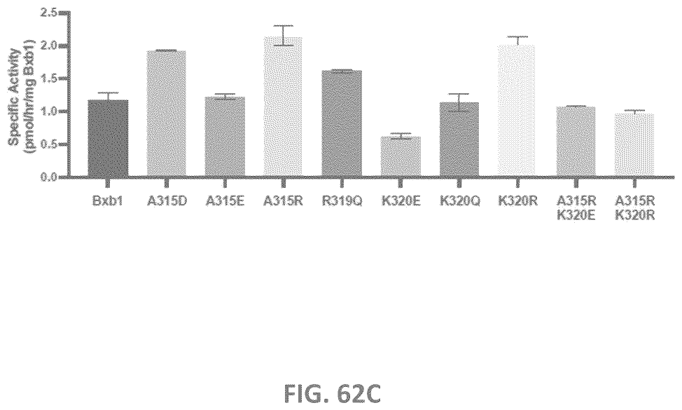

We characterized a series of these mutants biochemically, measuring both binding affinities and specific activities for mutations at A315 and K320. Many mutations did not significantly impact the interaction between Bxb1 and its substrates. However, in many cases any changes we engineered led to modest decreases in the affinity between Bxb1 and attB or attP (FIG. 62A). Interestingly, the mutation of K320 to glutamic acid greatly impacted the interaction. We observed that the affinity of Bxb1 for attB increased by ˜2-fold while the affinity for attP decreased by over 20-fold. Additionally, we observed an additive effect of reduced affinity for attB and attP in the A315R and K320R double mutant.

We also investigated the impact of mutations in the zinc ribbon domain on Bxb1's offbeacon activity. Here we looked at two different identified sites, CAS031 and CAS421, which is the most frequent off-beacon insertion site in human iPSCs (15). Using two sites allowed us to probe the effect of different mutations more thoroughly.

All tested mutations in the zinc ribbon domain led to a decrease in binding affinity (increase in KD) relative to wild-type Bxb1 binding to off-beacon sequences. (FIG. 62B). However, some mutations had a more significant effect on one or the other off-beacon sequences. For instance, K320E led to a 2.1-fold weaker KD for CAS031 compared to wildtype Bxb1 and a 4.6-fold weaker KD for CAS421. Conversely, A315R had minimal effect on binding to CAS421 compared to wild-type Bxb1 but led to a 2.3-fold weaker KD for CAS031. The intermediate effect on binding affinity observed in the A315R/K320E double mutant suggests that DNA recognition by Bxb1 is more complex than can be predicted by simple mutations.

We measured the specific activity for the same series of mutants but did not see any significant differences for many of the constructs (FIG. 62C). Even for K320E, a modest reduction in the specific activity was observed. However, it is likely that under conditions of limiting substrates a more pronounced effect on activity may be seen for K320E. While A315R or K320R mutations led to an increase in activity, no additive effect was observed in the double mutant but rather a decrease in specific activity was detected. Overall, the A315R single mutation led to the greatest increase in activity.

### 7.25.3.4 Naturally Occurring Variants of Bxb1 are as Active as Wild-Type Bxb1 on Bxb1 Substrates

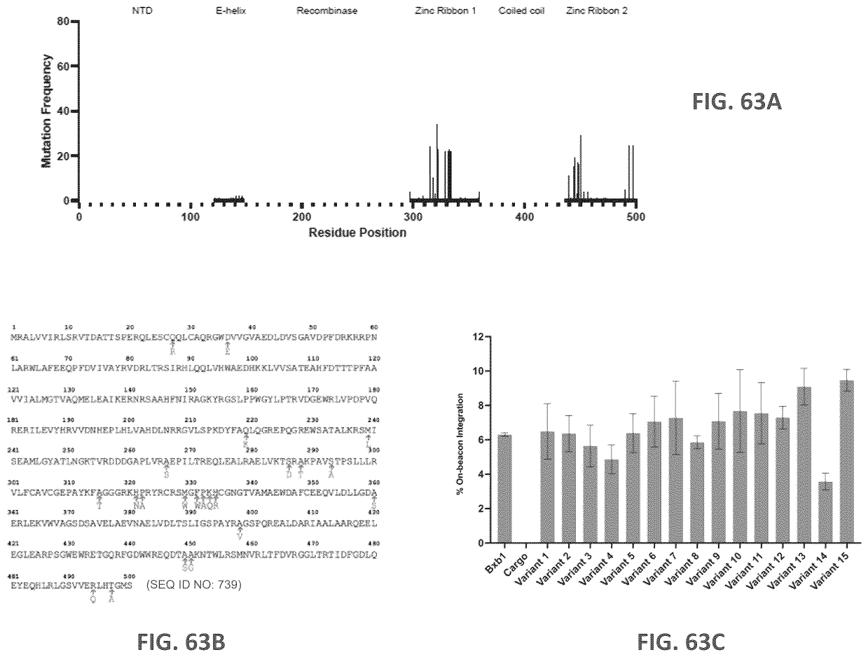

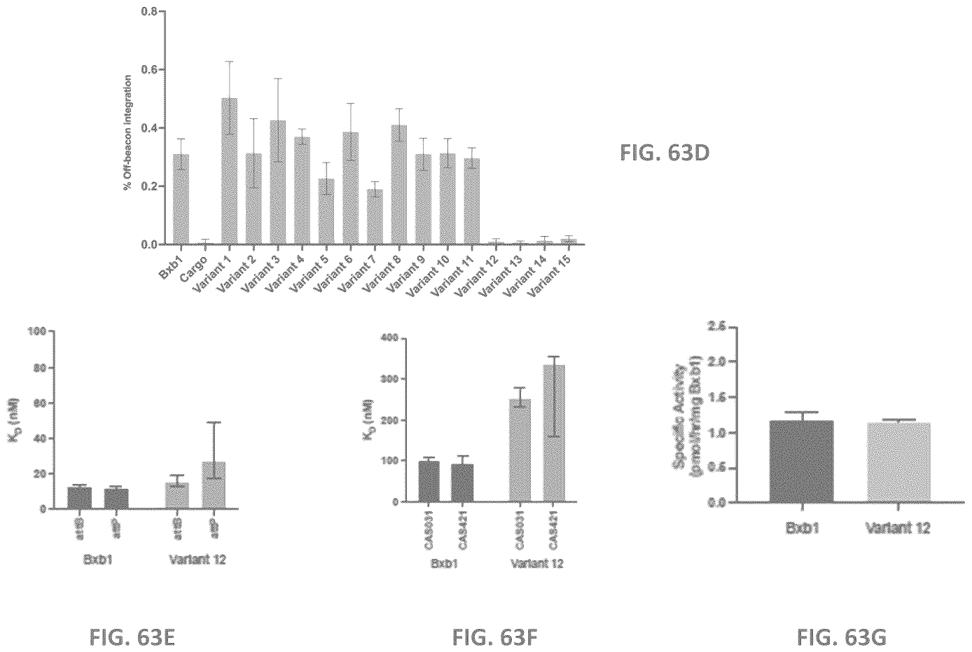

There are almost 200 sequences deposited in the NCBI database that share 90% sequence similarity to Bxb1. These sequences deviate from Bxb1 at positions through the protein sequence (FIG. 63A). However, they are enriched within the DNA binding domains of Bxb1. In fact, very few positions in the NTD or coiled-coil seem to differ from the canonical Bxb1 sequence. While we have not mapped the native attB or attP sequences for these new LSIs, we decided to assess whether any of these had activity on the original Bxb1 substrates.

Using a HEK293FT cell line with a pre-installed beacon, we assessed the activity of fifteen naturally occurring Bxb1 variants. These variant proteins had between one and nine differences from wild-type Bxb1, distributed throughout the protein sequence (FIG. 63B). Interestingly, most of the naturally occurring variants, unlike our designed variants, were at least as active, if not more active, than wild-type Bxb1 (FIG. 63C). We observed the biggest differences with mutations clustered in the zinc ribbon domain.

We also probed these naturally occurring variants for their off-beacon integration activity at CAS031. Unlike the engineered Bxb1 enzymes, most of these variants retained significant off-beacon activity (FIG. 63D). However, variants 12, 13 and 15 showed on beacon activity comparable to wild-type Bxb1 and exhibited low or no activity off-beacon at CAS031. All of these variants contain the same constellation of mutations in the zinc ribbon domain (A315T, H321N, P322A, M329W, F331W, P332A, K333Q, H334R). Variant 13 also contains S293A and variant 15 contains A360S.

We measured the binding affinity and specific activity for variant 12. We saw very subtle differences in binding affinity for wild-type attB or attP (FIG. 63E). We also measured binding of variant 12 to both CAS031 and CAS421, where we saw a significantly weaker binding affinity to both sequences compared to wild-type Bxb1 (FIG. 63F). We did not see any difference in the specific activity between variant 12 and wild-type Bxb1 (FIG. 63G). However, many of the altered positions between the two proteins would likely mediate interactions between the DNA and the LSI. Additionally, the natural substrates for these variant Bxb1 proteins may be different from those of canonical Bxb1.

### 7.25.3.5 Engineered Bxb1 Variants Improve Bxb1 Activity in Vivo

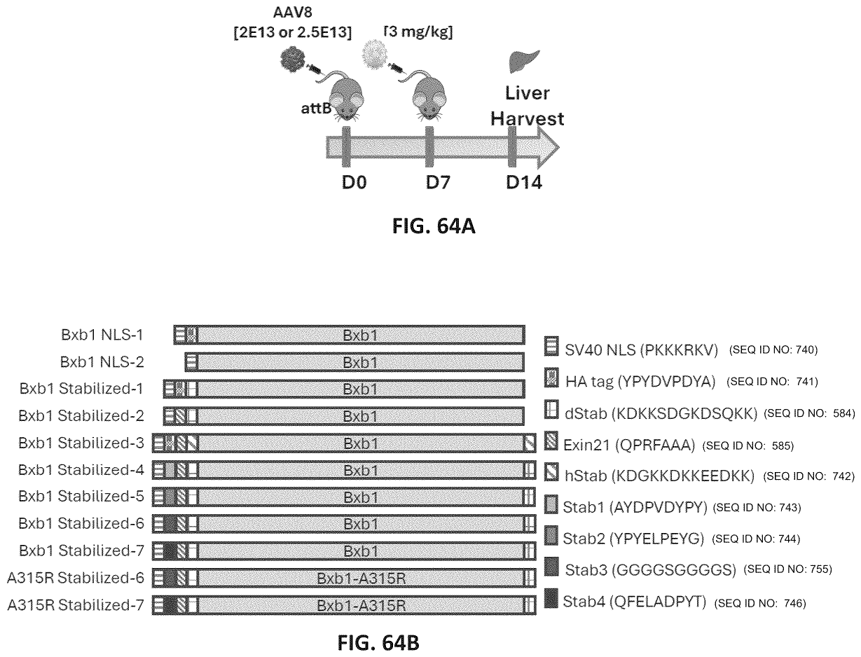

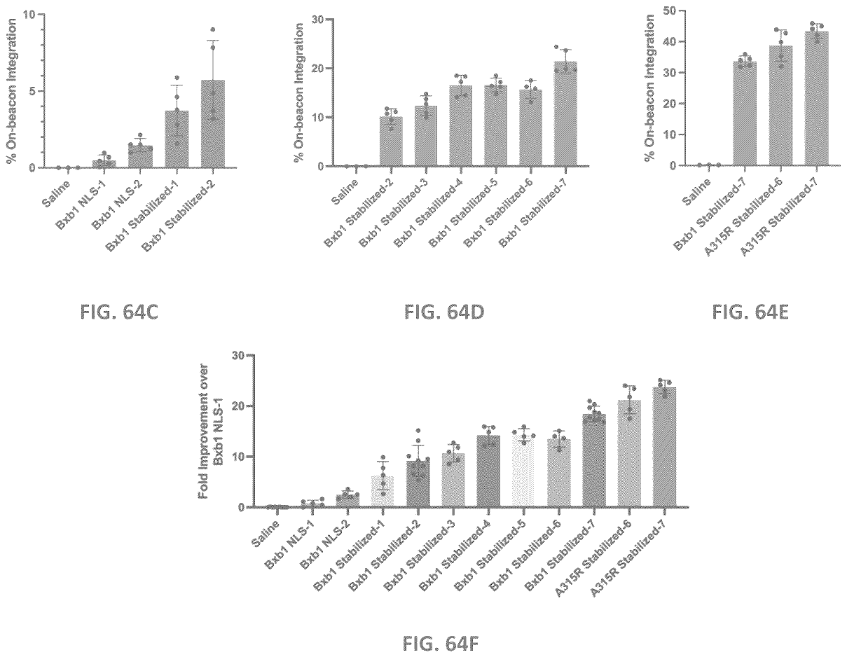

For genome engineering efforts, LSIs must be active in vivo. Using transgenic mice that had been generated to carry an attB in their genome, we assessed the activity of engineered Bxb1 variants on hepatocytes in vivo (FIG. 64A). Delivery was facilitated by an IV injection of an LNP carrying the mRNA encoding the LSI and template DNA was delivered by a selfcomplementary AAV8 contain the coding sequence for the human F9 gene. Our early studies found that Bxb1 showed only modest activity in vivo, with wild-type Bxb1 (containing a nuclear localization signal (NLS) and an HA-tag or just an NLS) resulting in only 1-2% integration (FIG. 64B, Bxb1NLS-1, Bxb1 NLS-2). We decided to apply traditional protein engineering efforts to improve this, namely by potentially increasing the stability of the protein through addition of stability tags. An N-terminal fusion with a STABILON tag (derived from the p54/Rpn10 ubiquitin receptor subunit of the Drosophila proteosome) (22) (FIG. 64B, Bxb1 Stabilized-1) increased in vivo potency almost 3-fold. Combining the STABILON tag with a second published sequence, Exin21 (FIG. 64B, Bxb1 Stabilized-2) further increased in vivo potency (23). Combining these two tags on the N-terminus of Bxb1 NLS-1 lead to over 5% gene insertion. Following some other optimizations of components, we were able to achieve 10% insertion with Bxb1 Stabilized-2 (FIG. 64C). Addition of a second STABILON sequence to the C-terminus (Bxb1 Stabilized-3) had a modest effect on the activity of Bxb1 in vivo (FIG. 64D). However, we had also modified the identity of the STABILON tag to the sequence from the human proteasome subunit, which may have led to lower activity overall (22). Returning to the Drosophila STABILON sequence, we altered the sequence N-terminal of the Exin21 tag to remove the HA-tag. Bxb1 Stabilized-4-7 variants all have a series amino acids containing charged or polar residues to act as an alternative to the HA-tag. For most of these we detected an improvement over the original stabilized sequence (Stabilized Bxb1-2) although these proteins also contain an additional STABILON at the C-terminus. For Bxb1 Stabilized-7, the rate of cargo integration is almost double compared to Bxb1 Stabilized-2.

Finally, we combined our best stabilized versions of Bxb1 with our engineered Bxb1 variants. The most active engineered Bxb1 was A315R and we combined this with two of the stabilized variants of Bxb1. We were able to achieve over 40% cargo integration in bulk liver tissue using Stabilized-A315R-7 (FIG. 64E). From Bxb1 NLS-1 to our final construct, we were able to improve in vivo potency of the integrase by over 25-fold (FIG. 64F).

### 7.25.4. Discussion

The ability to specifically place a large piece of DNA into a specific location has the potential to revolutionize genetic medicines. It can enable advanced cell engineering, as well as endogenous gene replacement for patients suffering from genetic diseases. Programmable Genomic Integration (PGI) based on LSIs has the potential to efficiently overcome the limitations of previous gene insertion technologies, however LSIs found in nature have challenges associated with potency and specificity. Here we described the development of a suite of novel integrases that could begin to address some of the fundamental issues with the current editing technologies.

One of the concerns with integrases is a lack of specificity (15). If gene insertion were to occur within a deleterious genomic site (e.g. tumor suppressor), it could lead to an unfavorable safety profile. The tumor suppressor gene would likely now be inactive and could result in development of malignancies. Our group and others have validated at least 50 sites of off-beacon integration by wild-type Bxb1 in mammalian cells, with many more potential sites showing detectable activity in cell-free biochemical assays (7,9,15).

To address this specificity issue, we have explored two different approaches. First, we used structure-based design to make specific point mutations in the zinc ribbon of Bxb1. Because Bxb1 seems to have fewer sequence-specific contacts in this part of the protein we hypothesized that we would be able to introduce mutations that would increase the stringency of binding to the substrates. We found several positions (notably A315 and K320) where mutation had a small or no effect on Bxb1 on-target activity in HEK293FT cells—leading to similar integration at on-beacon sites and decreased integration at off-beacon sites compared to wild-type Bxb1. These mutants positively impacted DNA binding by Bxb1, leading to proteins with higher affinity for the wild type sequence and lower affinity for the off-target sequences.

We also analyzed deposited Bxb1-like sequences to find common mutations that may be beneficial to enzyme activity due to their enrichment in these orthologues. We found one constellation of mutations that appeared to be quite beneficial for Bxb1 activity—increasing the level of on-beacon integration in HEK293FT cells while decreasing the level of off-beacon integration to virtually undetectable levels.

Large serine integrases also suffered from low levels of activity in vivo. Other groups have increased Bxb1's activity using protein evolution schemes to introduce mutations throughout the sequence to achieve efficient integration (7,13). We have used a different method, notably protein tagging with stabilizing sequences. This has led to significant improvements in Bxb1 activity—from less than 2% integration for unstabilized Bxb1 to over 40% in vivo integration for the fully stabilized constructs. This level of integration is now sufficient to be considered for use in therapeutic genome engineering using LSIs.

Bxb1 has historically been the integrase of choice for genomic engineering efforts (12). Here we have described improvements to Bxb1 to increase specificity and activity that will enable it to continue to lead the field for large serine integrases.

### 7.25.5. Method Details

### 7.25.5.1 Protein Purification

Bxb1 residues 2-500 and all mutants were cloned into a pET28 expression vector containing a Thioredoxin tag, CL7 affinity tag, 8× His (SEQ ID NO: 751) affinity tag, and SUMO tag (Trialtus Biosciences 20-1022). Protein was expressed in BL21(DE3) E. coli cells (New England Biolabs) grown in Terrific Broth (Teknova T7660) and induced with 1 mM IPTG for 16 hours at 18° C. The resulting cell pellet was resuspended in Ni NTA Buffer A with Halt Protease Inhibitor (Thermo Scientific 78439) and lysed by sonication. Crude lysate was centrifuged at 30,000×g for 30 min and the resulting cleared lysate applied to a 5 mL HisTrap HP column (Cytiva 17-5255-01). The column was washed with Ni NTA Buffer A: 50 mM Tris pH 8.0, 500 mM Sodium Chloride, 25 mM Imidazole, 1 mM DTT and eluted by a gradient with Ni NTA Buffer B: 500 mM Sodium Chloride, 250 mM Imidazole, 1 mM DTT pH 8.0. Following elution Arginine was added to reach a concentration of 200 mM and the protein was cleaved with 50 units of SUMO Protease (Trialtus Biosciences 30-1130) for 16 hours at 4° C. and exchanged into Heparin Buffer A with a HiPrep 26/10 Desalting column (Cytiva 17-5087-01). Cleaved enzyme was bound to a 5 mL HiTrap Heparin HP column (Cytiva 17-0407-03) and washed with Heparin Buffer A: 50 mM Tris pH 8.0, 100 mM Sodium Chloride, 200 mM Arginine, 1 mM TCEP and eluted by gradient elution with Heparin Buffer B: 50 mM Tris pH 8.0, 1 M Sodium Chloride, 200 mM Arginine, 1 mM TCEP. Final polishing was performed using a HiLoad Superdex 200 16/600 column (Cytiva 28-9893-36) and the buffer 50 mM Tris, 500 mM Sodium Chloride, 200 mM Arginine, 1 mM TCEP.

### 7.25.5.2 Biochemical Measurement of Bxb1

Substrates for biochemical assays were generated using single stranded gBlocks (IDT) designed for each specific target. Gel shift binding assays used a Cy3 or Cy5 fluorophore added to the 3′ end using a fluorescent tagged primer, biochemical activity assays used the same substrates with no fluorophore added.

Gel shift binding experiments were carried out with 1 nM substrate for KD values above 10 nM and 0.5 nM substrate for KD values below 10 nM. Gel shift binding buffer consisted of 20 mM Tris pH 7.4, 150 mM KCl, 2 mM DTT, 25 mM BSA, 25 mM Salmon Sperm DNA, 5% Glycerol. Enzyme and substrate mixtures were incubated for 30 minutes at 30° C. and separated using 6% Tris-Glycine PAGE gels (Thermo Scientific XP00062BOX). Gels were imaged using a iBright FL1500 gel imager (Thermo Scientific AF44115) and densitometry was performed using iBright Analysis Software (Thermo Scientific).

Activity assays were carried out with 20 nM of each substrate in a buffer consisting of 25 mM Tris pH 8.0, 100 mM KCl, 50 mM NaCl, 1 mM Spermidine, 5 mM MgCl2, 2.5 mM DTT, 5% Glycerol. Enzyme and substrate mixtures were incubated for 1 hour at 30° C. and reactions were stopped by addition of Proteinase K and incubation at 37° C. for 30 minutes. Samples were visualized using a 4200 Tapestation (Agilent G2991BA) using D1000 DNA Screen Tapes (Agilent, 5067-5582). Area under the curve measurements for the resulting bands were used to calculate the fraction of each substrate/product. Specific activity calculations were performed using the concentration of enzyme where the reaction had proceeded to 20% completeness.

### 7.25.5.3 In Vitro Transcription of mRNA

All studied genes were cloned into the vector backbone for in vitro transcription (IVT) that contains a single copy of the 5′ UTR and the 3′ UTR from the Xenopus laevis beta globin gene, in addition to a 110 nt polyA tail (SEQ ID NO: 723). Plasmid DNA containing coding sequences were linearized using a BspQI restriction site located immediately downstream of the polyA tail. Linearized plasmids were then purified via AMPure XP magnetic bead (Beckman Coulter A63882).

All mRNAs were generated via IVT reactions using the T7 RNA polymerase. mRNA was capped cotranscriptionally with CleanCap Reagent AG (TriLink BioTech N-7113-100). Each reaction contained final concentration of ˜50 μg/mL for linearized DNA template, 1× reaction buffer (Hongene Biotech ON-062), 15 mM of MgCl2, 10 units/μL of T7 RNA polymerase (Hongene Biotech ON-004), 0.002 units/μL of yeast inorganic pyrophosphatase (Hongene Biotech ON-025), 1 unit/μL of murine ribonuclease inhibitor (Hongene Biotech ON-039), 5 mM of CleanCap Reagent AG (TriLink BioTech N-7113-100), and 5 mM of each NTP (Jena Bioscience NU-1010, Hongene Biotech R3-056, Hongene Biotech R2-057). In each IVT reaction, UTP was swapped for N1-methylpseudouridine-5′-triphosphate (BOC Sciences 1428903-59-6). IVT reactions were incubated at 37° C. for 2 hours, followed by DNase I digestion of the template DNA (Hongene Biotech ON-109). mRNA products were purified using an RNA Maxi Prep kit (Qiagen), quantified using a NanoDrop (Thermo Fisher Scientific), and checked for integrity using a Fragment Analyzer (Agilent).

### 7.25.5.4 Generation of attB+HEK293FT Cell Line

In-house produced lentivirus containing a transfer plasmid with an EFlα-PuroR-WPRE backbone carrying a 46 bp Bxb1 attB insert was transduced into HEK293FT cells (Thermo Fisher Scientific R70007). Cells with Low MOI were plated in sterile 96-well tissue culture plates under puromycin selection via serial dilutions for clone selection. After clone selection, single lentiviral insertion on chromosome 5 was confirmed using ligation mediated PCR with primers targeting the 5′ and 3′ LTRs along with Cergentis TLA mapping (data not shown).

### 7.25.5.5 General Cell Culture Conditions

The attB+HEK293FT cells were cultured in DMEM (Corning 10-013-CV) with 10% FBS (Gibco A3160501) and 1 μg/mL puromycin under standard tissue culture conditions (at 37° C. and 5% CO2).

Cells were detached for splitting and plating using TrypLE Express (Thermo Fisher Scientific 12604039) according to the manufacturer's instructions.

### 7.25.5.6 Transfection Conditions

To transfect cells with plasmids and mRNAs, Lipofectamine MessengerMax (Thermofisher LMRNA001) was used as transfection reagent. 1 d before transfection, cells were seeded onto 96-well tissue culture plates at a density of 21,000 cells per well. For the on-beacon integration assay, 30 ng of Bxb1 variant mRNA and 3.5 ng of cargo plasmid were mixed with 0.3 uL of LipofectamineMessengerMax and co-transfected into cells plated in a single well. For the off-beacon integration assay, 100 ng of Bxb1 variant mRNA and 60 ng of cargo plasmid were mixed with 0.3 uL of Lipofectamine MessengerMax and co-transfected into cells plated in a single well.

### 7.25.5.7 Genomic DNA Extraction

Genomic DNA (gDNA) was extracted 3 d after transfection by removing medium, resuspending cells in 50 uL QuickExtract (LGC Biosearch Technologies QE0905T), and incubating at 75° C. for 10 min followed by 95° C. for 5 min. Then gDNA was purified from cell lysates by AMPure XP magnetic bead (Beckman Coulter A63882).

### 7.25.5.8 Droplet Digital Polymerase Chain Reaction (ddPCR)

Custom primers and probes were designed to measure editing in the studied loci. Probes were dual labelled with 3′-3IABkFQ and either 5′-carboxyflurescein (FAM) for edit targets or 5′-hexachlorofluorescein phosphoramidite (HEX) for reference genes. From each sample, ddPCR signals from the edit target assay and reference assay were collected together. Percent integration was calculated by dividing the number of FAM-positive droplets (indicating successful cargo integration) by the total number of HEX-positive droplets (reference gene) and then converting the values to a percentage. Assays were validated using gBlocks representing edit outcomes to test for both specificity and linearity. All primers, probes, and gBlocks were synthesized by Integrated DNA Technologies (IDT).

Each reaction contained 11 μL of 2× ddPCR Supermix for probes (No dUTP) (Bio-Rad 1863025), final concentration of 0.5 μM for each primer and 0.25 μM for each probe, 0.11 μL each of HindIII and Eco91I (Thermo Fisher Scientific FD0504 and FD0394, respectively) ˜100 ng of gDNA and water to a final volume of 22 μL. Droplets were generated on the AutoDG Instrument for automated droplet generation (Bio-Rad 1864101). PCR amplification was performed with the following cycling parameters: initial enzyme activation at 94° C. for 10 min, followed by 40 cycles of denaturation at 94° C. for 30 s and combined annealing/extension step at 58° C. for 1 min, and a final step at 98° C. for 5 min. Data acquisition and analysis were performed on the QX200 Droplet Reader (Bio-rad 1864003).

### 7.25.5.9 In Vivo Mouse Studies

All animal study procedures were approved by Explora BioLabs under IACUC protocol EB17-004-302. Transgenic C57BL/6J mice with a knock-in of the attB site in intron 1 of F9 or Rosa26 were generated by Biocytogen (Beijing, China) using CRISPR/Cas9. Mice were transferred to Biomere (Worcester, MA, USA) for breeding. AAV8 (AAVG107) cargo was intravenously injected into adult mice (-20-46 weeks) at a dose of 2E13 or 2.5E13 at day zero. At day seven, LNPs formulated with corresponding Bxb1 variant mRNAs were intravenously injected into the mice at a dose of 3 mg/kg. On day fourteen post LNP injection, animals were euthanized, and liver tissue was collected from the median lobe from each animal and homogenized on Precellys Evolution (cat K002198-PEVOO-A.0 Combo, Bertin technologies, WA, USA).

### 7.25.5.10 Liver gDNA Isolation and Analysis

Liver gDNA was extracted with quick-DNA/RNA MagBead kit (cat R2131, Zymo research, CA, USA) and analyzed by ddPCR using BioRad Automated Droplet Generator (cat 1864101) and BioRad QX200 Droplet Reader (cat 1864003). Approximately 30 ng of input gDNA were loaded into all ddPCR reactions. Restriction enzymes Eco911 and HindIII were added at [1:200] dilution to ddPCR master mixes. I-PGI attL and attR analyses were performed using the following ddPCR primer-probes: mF9 attB (attL: Fwd primer (5′-GGAGTCCAACCTTAAGTCTCTTG-3′(SEQ ID NO: 706)), Rev primer (5′-GGTTCAGGATCTTGTTGGCG-3′ (SEQ ID NO: 707)), probe (5′-FAM CTTGTCGACGACGGCGGTCTCAGTG-3′(SEQ ID NO: 697)); attR: Fwd primer (5′-GCAACTTGGGCTACCAGAT-3′(SEQ ID NO: 708)), Rev primer (5′-GCTGTTTTGTTTCCACTGTGTAC-3′(SEQ ID NO: 709)), probe (5′-FAM CGGTCTCCGTCGTCAGGATCAT-3′(SEQ ID NO: 710)), Rosa26 attB: (attL: Fwd primer (5′-AGTATACCAGCTAGCAGGCC-3′(SEQ ID NO: 711)), Rev primer (5′-GGTTCAGGATCTTGTTGGCG-3′(SEQ ID NO: 707)), probe(5′-FAM CTTGTCGACGACGGCGGTCTCAGTG-3′(SEQ ID: 697)); attR: Fwd primer (5′-GCAACTTGGGCTACCAGAT-3′(SEQ ID NO: 708)), Rev primer (5′-GCAGAGAACTCCCAGAAAGGT-3′(SEQ ID NO: 712)), probe (5′-FAM CGGTCTCCGTCGTCAGGATCAT-3′ (SEQ ID NO: 710)). mTfrc probe (Applied Biosciences cat 4458370) was used as reference.

### 7.25.6. Materials

Table 27 below, related to FIGS. 60B-60E, 62A-62C, 63E-63G DNA substrates for biochemical assays.

TABLE 27

| | | SEQ |
|---|---|---|
| | | ID |
| Name | Sequence | Nos |
| Bxb1 attB | CGAGTGGTGCGACGTCTGGCTTTCGGTGCCCTAGTGATGAGAAATATG | 679 |
| 180 bp | TATTTCGTTCTTGCAGCTTGTCAGTACTTTCAGAATCATGGTCTGCATG | |
| | GTAGAATGGCCGGCTTGTCGACGACGGCGGTCTCCGTCGTCAGGATCA | |
| | TCCGGTCGCTATCTAGCGAGTTCGCGCGCTCTGTG | |
| Bxb1 attP | GTGTAGCGTCGCTACGCGCCATTGTCAGCACAAGAtgGGTTTGTCTGGT | 680 |
| 220 bp | CAACCACCGCGGTCTCAGTGGTGTACGGTACAAACCacCATGAAGGCTA | |
| | GAATTACTTACCGGCCTTTACCATGCCTGCACTATACGCACCTACTTTC | |
| | CCGTTTATCTGTCCAAGCAGATACAATGCGATCCTCCGTTAAGATATTC | |
| | TTACGTGCGACGTCGCCGTGGATG | |
| Bxb1 attL | CGAGTGGTGCGACGTCTGGCTTTCGGTGCCCTAGTGATGAGAAATATG | 681 |
| 286 bp | TATTTCGTTCTTGCAGCTTGTCAGTACTTTCAGAATCATGGTCTGCATG | |
| | GTAGAATGGCCGGCTTGTCGACGACGGCGGTCTCAGTGGTGTACGGTA | |
| | CAAACCCACATGAAGGCTAGAATTACTTACCGGCCTTTACCATGCCTG | |
| | CACTATACGCACCTACTTTCCCGTTTATCTGTCCAAGCAGATACAATGC | |
| | GATCCTCCGTTAAGATATTCTTACGTGCGACGTCGCCGTGGATG | |
| Bxb1 attR | GTGTAGCGTCGCTACGCGCCATTGTCAGCACAAGAGTGGTTTGTCTGG | 682 |
| 115 bp | TCAACCACCGCGGTCTCCGTCGTCAGGATCATCCGGTCGCTATCTAGC | |
| | GAGTTCGCGCGCTCTGTG | |
| Bxb1 | CGAGTGGTGCGACGTCTGGCTTTCGGTGCCCTAGTGATGAGAAATATG | 683 |
| CAS031 | TATTTCGTTCTTGCAGCTTGTCAGTACTTTCAGAATCATGGTCTGCATG | |
| | GTAGAATAAGCACGACCTCCACAACAGAGGTTGCTGTTGTGGAGGTTG | |
| | CTCAATCGCTATCTAGTTGAGTAAGAGCTGGCGAGTTCGCGCGCTCTGT | |
| | G | |
| Bxb1 | CGAGTGGTGCGACGTCTGGCTTTCGGTGCCCTAGTGATGAGAAATATG | 684 |
| CAS421 | TATTTCGTTCTTGCAGCTTGTCAGTACTTTCAGAATCATGGTCTGCATG | |
| | GTAGAATGTTTGGTTTGTTTGCAACGGCAGTGACGGAGGTTGGGAGCC | |
| | AGGCTTCGCTATCTAGTTGAGTAAGAGCTGGCGAGTTCGCGCGCTCTG | |
| | TG | |
| Bxb1 | CGAGTGGTGCGACGTCTGGCTTTCGGTGCCCTAGTGATGAGAAATATG | 685 |
| attB_40 bp | TATTTCGTTCTTGCAGCTTGTCAGTACTTTCAGAATCATGGTCTGCATG | |
| 180 bp | GTAGAATttaCGGCTTGTCGACGACGGCGGTCTCCGTCGTCAGGATCATC | |
| | attTCGCTATCTAGCGAGTTCGCGCGCTCTGTG | |
| Bxb1 | CGAGTGGTGCGACGTCTGGCTTTCGGTGCCCTAGTGATGAGAAATATG | 686 |
| attB_38 bp | TATTTCGTTCTTGCAGCTTGTCAGTACTTTCAGAATCATGGTCTGCATG | |
| 180 bp | GTAGAATttaaGGCTTGTCGACGACGGCGGTCTCCGTCGTCAGGATCATaa | |
| | ttTCGCTATCTAGCGAGTTCGCGCGCTCTGTG | |
| Bxb1 | CGAGTGGTGCGACGTCTGGCTTTCGGTGCCCTAGTGATGAGAAATATG | 687 |
| attB_36 bp | TATTTCGTTCTTGCAGCTTGTCAGTACTTTCAGAATCATGGTCTGCATG | |
| 180 bp | GTAGAATttaatGCTTGTCGACGACGGCGGTCTCCGTCGTCAGGATCAgaatt | |
| | TCGCTATCTAGCGAGTTCGCGCGCTCTGTG | |
| Bxb1 | CGAGTGGTGCGACGTCTGGCTTTCGGTGCCCTAGTGATGAGAAATATG | 688 |
| attB_34 bp | TATTTCGTTCTTGCAGCTTGTCAGTACTTTCAGAATCATGGTCTGCATG | |
| 180 bp | GTAGAATttaattCTTGTCGACGACGGCGGTCTCCGTCGTCAGGATCcgaattT | |
| | CGCTATCTAGCGAGTTCGCGCGCTCTGTG | |
| Bxb1 | CGAGTGGTGCGACGTCTGGCTTTCGGTGCCCTAGTGATGAGAAATAGT | 689 |
| attB_32 bp | ATTTCGTTCTTGCAGCTTGTCAGTACTTTCAGAATCATGGTCTGCATGG | |
| 180 bp | TAGAATttaattaTTGTCGACGACGGCGGTCTCCGTCGTCAGGATacgaattTCG | |
| | CTATCTAGCGAGTTCGCGCGCTCTGTG | |
| Bxb1 | CGAGTGGTGCGACGTCTGGCTTTCGGTGCCCTAGTGATGAGAAATATG | 690 |
| attB_30 bp | TATTTCGTTCTTGCAGCTTGTCAGTACTTTCAGAATCATGGTCTGCATG | |
| 180 bp | GTAGAATttaattagTGTCGACGACGGCGGTCTCCGTCGTCAGGAgacgaattTC | |
| | GCTATCTAGCGAGTTCGCGCGCTCTGTG | |
| attB 5′ F | CGAGTGGTGCGACGTCTGGCattB 3′ R | 691 |
| attB 3′ R | GCGAGTTCGCGCGCTCTGTG | 692 |
| attP 5′ F | GTGTAGCGTCGCTACGCGCC | 693 |
| attP 3′ R | CATCCACGGCGACGTCGCAC | 694 |

Table 28 below, related to FIGS. 61C-61D, 63C-63D. DNA primers and probes for in vitro ddPCR assay.

TABLE 28

| | | SEQ |
|---|---|---|
| | Sequence (5′→3′) | ID Nos. |
| On-beacon, | TACCATCCACTCGACA | 695 |
| target forward | CACC | |
| On- & Off-beacon, | GAACTCCACGCCGTTC | 696 |
| target reverse | A | |
| On-beacon, | CTTGTCGACGACGGCG | 697 |
| target probe | GTCTCAGTG | |
| On-beacon, | GCTTTGTTCCTTGGGT | 698 |
| reference forward | TCTTG | |
| On-beacon, | TTCTGCTGCTGCACTA | 699 |
| reference reverse | TACC | |
| On-beacon, | AATTGTCTGGCCTGTA | 700 |
| reference probe | CCGTCAGC | |
| Off-beacon, | GTAAGAGCCTGGCTTA | 701 |
| target forward | ATGAGC | |
| Off-beacon, | CCTCCACAACAGAGGT | 702 |
| target probe | CTCAGTGGTGTACG | |
| Off-beacon, | GTGATGTTGTAAGTTT | 703 |
| reference forward | TCCACAGC | |
| Off-beacon, | GGAGCTTAGCAATCTC | 704 |
| reference reverse | TCTGATTT | |
| Off-beacon, | AGACTTCCCTTTCTTC | 705 |
| reference probe | TGCCACTGCCAC | |

Table 29 below, related to FIG. 63C. Protein mutations of studied Bxb1 variants

TABLE 29

| | |
|---|---|
| Bxb1 (WT) | Bxb1 Stabilized-1 |
| Bxb1 Variant 1 | Q27R |
| Bxb1 Variant 2 | D36E |
| Bxb1 Variant 3 | Q219K, M239L |
| Bxb1 Variant 4 | Q219K, M239L, S286D |
| Bxb1 Variant 5 | Q219K, M239L, S286D, A288T |
| Bxb1 Variant 6 | Q219K, M239L, A266S, S286D, A288T |
| Bxb1 Variant 7 | S293A |
| Bxb1 Variant 8 | A360S |
| Bxb1 Variant 9 | A398V |
| Bxb1 Variant 10 | A449S, A450G |
| Bxb1 Variant 11 | A449S, R494Q, T497A |
| Bxb1 Variant 12 | A315T, H321N, P322A, M329W, F331W, |
| | P332A, K333Q, H334R |
| Bxb1 Variant 13 | A315T, H321N, P322A, M329W, F331W, |
| | P332A, K333Q, H334R, S293A |
| Bxb1 Variant 14 | A315T, H321N, P322A, M329W, F331W, |
| | P332A, K333Q, H334R, K320E |
| Bxb1 Variant 15 | A315T, H321N, P322A, M329W, F331W, |
| | P332A, K333Q, H334R, A360S |

Table 30 below, related to FIG. 64B. Protein sequences for stabilized variants

TABLE 30

| | | |
|---|---|---|
| Bxb1 | MPKKKRKVYPYDVPDYAGSRALVVIRLSRVTDATTSPERQLESCQQLCAQR | SEQ ID |
| NLS-1 | GWDVVGVAEDLDVSGAVDPFDRKRRPNLARWLAFEEQPFDVIVAYRVDRL | NO: 713 |
| | TRSIRHLQQLVHWAEDHKKLVVSATEAHFDTTTPFAAVVIALMGTVAQMEL | |
| | EAIKERNRSAAHFNIRAGKYRGSLPPWGYLPTRVDGEWRLVPDPVQRERILE | |
| | VYHRVVDNHEPLHLVAHDLNRRGVLSPKDYFAQLQGREPQGREWSATALK | |
| | RSMISEAMLGYATLNGKTVRDDDGAPLVRAEPILTREQLEALRAELVKTSRA | |
| | KPAVSTPSLLLRVLFCAVCGEPAYKFAGGGRKHPRYRCRSMGFPKHCGNGT | |
| | VAMAEWDAFCEEQVLDLLGDAERLEKVWVAGSDSAVELAEVNAELVDLTS | |
| | LIGSPAYRAGSPQREALDARIAALAARQEELEGLEARPSGWEWRETGQRFGD | |
| | WWREQDTAAKNTWLRSMNVRLTFDVRGGLTRTIDFGDLQEYEQHLRLGSV | |
| | VERLHTGMS* | |
| Bxb1 | MPKKKRKVGSGRALVVIRLSRVTDATTSPERQLESCQQLCAQRGWDVVGVA | SEQ ID |
| NLS-2 | EDLDVSGAVDPFDRKRRPNLARWLAFEEQPFDVIVAYRVDRLTRSIRHLQQL | NO: 714 |
| | VHWAEDHKKLVVSATEAHFDTTTPFAAVVIALMGTVAQMELEAIKERNRSA | |
| | AHFNIRAGKYRGSLPPWGYLPTRVDGEWRLVPDPVQRERILEVYHRVVDNH | |
| | EPLHLVAHDLNRRGVLSPKDYFAQLQGREPQGREWSATALKRSMISEAMLG | |
| | YATLNGKTVRDDDGAPLVRAEPILTREQLEALRAELVKTSRAKPAVSTPSLLL | |
| | RVLFCAVCGEPAYKFAGGGRKHPRYRCRSMGFPKHCGNGTVAMAEWDAFC | |
| | EEQVLDLLGDAERLEKVWVAGSDSAVELAEVNAELVDLTSLIGSPAYRAGSP | |
| | QREALDARIAALAARQEELEGLEARPSGWEWRETGQRFGDWWREQDTAAK | |
| | NTWLRSMNVRLTFDVRGGLTRTIDFGDLQEYEQHLRLGSVVERLHTGMS* | |
| Bxb1 | MPKKKRKVYPYDVPDYAKDKKSDGKDSQKKGSRALVVIRLSRVTDATTSPE | SEQ ID |
| Stabi- | RQLESCQQLCAQRGWDVVGVAEDLDVSGAVDPFDRKRRPNLARWLAFEEQ | NO: 621 |
| lized-1 | PFDVIVAYRVDRLTRSIRHLQQLVHWAEDHKKLVVSATEAHFDTTTPFAAVV | |
| | IALMGTVAQMELEAIKERNRSAAHFNIRAGKYRGSLPPWGYLPTRVDGEWR | |
| | LVPDPVQRERILEVYHRVVDNHEPLHLVAHDLNRRGVLSPKDYFAQLQGREP | |
| | QGREWSATALKRSMISEAMLGYATLNGKTVRDDDGAPLVRAEPILTREQLE | |
| | ALRAELVKTSRAKPAVSTPSLLLRVLFCAVCGEPAYKFAGGGRKHPRYRCRS | |
| | MGFPKHCGNGTVAMAEWDAFCEEQVLDLLGDAERLEKVWVAGSDSAVEL | |
| | AEVNAELVDLTSLIGSPAYRAGSPQREALDARIAALAARQEELEGLEARPSG | |
| | WEWRETGQRFGDWWREQDTAAKNTWLRSMNVRLTFDVRGGLTRTIDFGD | |
| | LQEYEQHLRLGSVVERLHTGMS* | |
| Bxb1 | MPKKKRKVQPRFAAAGSGKDKKSDGKDSQKKGSGRALVVIRLSRVTDATTS | SEQ ID |
| Stabi- | PERQLESCQQLCAQRGWDVVGVAEDLDVSGAVDPFDRKRRPNLARWLAFE | NO: 625 |
| lized-2 | EQPFDVIVAYRVDRLTRSIRHLQQLVHWAEDHKKLVVSATEAHFDTTTPFAA | |
| | VVIALMGTVAQMELEAIKERNRSAAHFNIRAGKYRGSLPPWGYLPTRVDGE | |
| | WRLVPDPVQRERILEVYHRVVDNHEPLHLVAHDLNRRGVLSPKDYFAQLQG | |
| | REPQGREWSATALKRSMISEAMLGYATLNGKTVRDDDGAPLVRAEPILTREQ | |
| | LEALRAELVKTSRAKPAVSTPSLLLRVLFCAVCGEPAYKFAGGGRKHPRYRC | |
| | RSMGFPKHCGNGTVAMAEWDAFCEEQVLDLLGDAERLEKVWVAGSDSAVE | |
| | LAEVNAELVDLTSLIGSPAYRAGSPQREALDARIAALAARQEELEGLEARPSG | |
| | WEWRETGQRFGDWWREQDTAAKNTWLRSMNVRLTFDVRGGLTRTIDFGD | |
| | LQEYEQHLRLGSVVERLHTGMS* | |
| Bxb1 | MPKKKRKVYPYDVPDYAQPRFAAAGSGKDGKKDKKEEDKKGSRALVVIRL | SEQ ID |
| Stabi- | SRVTDATTSPERQLESCQQLCAQRGWDVVGVAEDLDVSGAVDPFDRKRRPN | NO: 663 |
| lized-3 | LARWLAFEEQPFDVIVAYRVDRLTRSIRHLQQLVHWAEDHKKLVVSATEAH | |
| | FDTTTPFAAVVIALMGTVAQMELEAIKERNRSAAHFNIRAGKYRGSLPPWGY | |
| | LPTRVDGEWRLVPDPVQRERILEVYHRVVDNHEPLHLVAHDLNRRGVLSPK | |
| | DYFAQLQGREPQGREWSATALKRSMISEAMLGYATLNGKTVRDDDGAPLV | |
| | RAEPILTREQLEALRAELVKTSRAKPAVSTPSLLLRVLFCAVCGEPAYKFAGG | |
| | GRKHPRYRCRSMGFPKHCGNGTVAMAEWDAFCEEQVLDLLGDAERLEKV | |
| | WVAGSDSAVELAEVNAELVDLTSLIGSPAYRAGSPQREALDARIAALAARQE | |
| | ELEGLEARPSGWEWRETGQRFGDWWREQDTAAKNTWLRSMNVRLTFDVR | |
| | GGLTRTIDFGDLQEYEQHLRLGSVVERLHTGMSGSGKDGKKDKKEEDKK* | |
| Bxb1 | MPKKKRKVAYDPVDYPYQPRFAAAGSGKDKKSDGKDSQKKGSRALVVIRL | SEQ ID |
| Stabi- | SRVTDATTSPERQLESCQQLCAQRGWDVVGVAEDLDVSGAVDPFDRKRRPN | NO: 715 |
| lized-4 | LARWLAFEEQPFDVIVAYRVDRLTRSIRHLQQLVHWAEDHKKLVVSATEAH | |
| | FDTTTPFAAVVIALMGTVAQMELEAIKERNRSAAHFNIRAGKYRGSLPPWGY | |
| | LPTRVDGEWRLVPDPVQRERILEVYHRVVDNHEPLHLVAHDLNRRGVLSPK | |
| | DYFAQLQGREPQGREWSATALKRSMISEAMLGYATLNGKTVRDDDGAPLV | |
| | RAEPILTREQLEALRAELVKTSRAKPAVSTPSLLLRVLFCAVCGEPAYKFAGG | |
| | GRKHPRYRCRSMGFPKHCGNGTVAMAEWDAFCEEQVLDLLGDAERLEKV | |
| | WVAGSDSAVELAEVNAELVDLTSLIGSPAYRAGSPQREALDARIAALAARQE | |
| | ELEGLEARPSGWEWRETGQRFGDWWREQDTAAKNTWLRSMNVRLTFDVR | |
| | GGLTRTIDFGDLQEYEQHLRLGSVVERLHTGMSGSGKDKKSDGKDSQKK* | |
| Bxb1 | MPKKKRKVYPYELPEYGQPRFAAAGSGKDKKSDGKDSQKKGSRALVVIRLS | SEQ ID |
| Stabi- | RVTDATTSPERQLESCQQLCAQRGWDVVGVAEDLDVSGAVDPFDRKRRPNL | NO: 716 |
| lized-5 | ARWLAFEEQPFDVIVAYRVDRLTRSIRHLQQLVHWAEDHKKLVVSATEAHF | |
| | DTTTPFAAVVIALMGTVAQMELEAIKERNRSAAHFNIRAGKYRGSLPPWGYL | |
| | PTRVDGEWRLVPDPVQRERILEVYHRVVDNHEPLHLVAHDLNRRGVLSPKD | |
| | YFAQLQGREPQGREWSATALKRSMISEAMLGYATLNGKTVRDDDGAPLVR | |
| | AEPILTREQLEALRAELVKTSRAKPAVSTPSLLLRVLFCAVCGEPAYKFAGGG | |
| | RKHPRYRCRSMGFPKHCGNGTVAMAEWDAFCEEQVLDLLGDAERLEKVW | |
| | VAGSDSAVELAEVNAELVDLTSLIGSPAYRAGSPQREALDARIAALAARQEE | |
| | LEGLEARPSGWEWRETGQRFGDWWREQDTAAKNTWLRSMNVRLTFDVRG | |
| | GLTRTIDFGDLQEYEQHLRLGSVVERLHTGMSGSGKDKKSDGKDSQKK* | |
| Bxb1 | MPKKKRKVGGGGSGGGGSQPRFAAAGSGKDKKSDGKDSQKKGSRALVVIR | SEQ ID |
| Stabi- | LSRVTDATTSPERQLESCQQLCAQRGWDVVGVAEDLDVSGAVDPFDRKRRP | NO: 717 |
| lized-6 | NLARWLAFEEQPFDVIVAYRVDRLTRSIRHLQQLVHWAEDHKKLVVSATEA | |
| | HFDTTTPFAAVVIALMGTVAQMELEAIKERNRSAAHFNIRAGKYRGSLPPWG | |
| | YLPTRVDGEWRLVPDPVQRERILEVYHRVVDNHEPLHLVAHDLNRRGVLSP | |
| | KDYFAQLQGREPQGREWSATALKRSMISEAMLGYATLNGKTVRDDDGAPL | |
| | VRAEPILTREQLEALRAELVKTSRAKPAVSTPSLLLRVLFCAVCGEPAYKFAG | |
| | GGRKHPRYRCRSMGFPKHCGNGTVAMAEWDAFCEEQVLDLLGDAERLEKV | |
| | WVAGSDSAVELAEVNAELVDLTSLIGSPAYRAGSPQREALDARIAALAARQE | |
| | ELEGLEARPSGWEWRETGQRFGDWWREQDTAAKNTWLRSMNVRLTFDVR | |
| | GGLTRTIDFGDLQEYEQHLRLGSVVERLHTGMSGSGKDKKSDGKDSQKK* | |
| Bxb1 | MPKKKRKVQFELADPYTQPRFAAAGSGKDKKSDGKDSQKKGSRALVVIRLS | SEQ ID |
| Stabi- | RVTDATTSPERQLESCQQLCAQRGWDVVGVAEDLDVSGAVDPFDRKRRPNL | NO: 718 |
| lized-7 | ARWLAFEEQPFDVIVAYRVDRLTRSIRHLQQLVHWAEDHKKLVVSATEAHF | |
| | DTTTPFAAVVIALMGTVAQMELEAIKERNRSAAHFNIRAGKYRGSLPPWGYL | |
| | PTRVDGEWRLVPDPVQRERILEVYHRVVDNHEPLHLVAHDLNRRGVLSPKD | |
| | YFAQLQGREPQGREWSATALKRSMISEAMLGYATLNGKTVRDDDGAPLVR | |
| | AEPILTREQLEALRAELVKTSRAKPAVSTPSLLLRVLFCAVCGEPAYKFAGGG | |
| | RKHPRYRCRSMGFPKHCGNGTVAMAEWDAFCEEQVLDLLGDAERLEKVW | |
| | VAGSDSAVELAEVNAELVDLTSLIGSPAYRAGSPQREALDARIAALAARQEE | |
| | LEGLEARPSGWEWRETGQRFGDWWREQDTAAKNTWLRSMNVRLTFDVRG | |
| | GLTRTIDFGDLQEYEQHLRLGSVVERLHTGMSGSGKDKKSDGKDSQKK* | |
| A315R | MPKKKRKVGGGGSGGGGSQPRFAAAGSGKDKKSDGKDSQKKGSRALVVIR | SEQ ID |
| Stabi- | LSRVTDATTSPERQLESCQQLCAQRGWDVVGVAEDLDVSGAVDPFDRKRRP | NO: 719 |
| lized-6 | NLARWLAFEEQPFDVIVAYRVDRLTRSIRHLQQLVHWAEDHKKLVVSATEA | |
| | HFDTTTPFAAVVIALMGTVAQMELEAIKERNRSAAHFNIRAGKYRGSLPPWG | |
| | YLPTRVDGEWRLVPDPVQRERILEVYHRVVDNHEPLHLVAHDLNRRGVLSP | |
| | KDYFAQLQGREPQGREWSATALKRSMISEAMLGYATLNGKTVRDDDGAPL | |
| | VRAEPILTREQLEALRAELVKTSRAKPAVSTPSLLLRVLFCAVCGEPAYKFRG | |
| | GGRKHPRYRCRSMGFPKHCGNGTVAMAEWDAFCEEQVLDLLGDAERLEKV | |
| | WVAGSDSAVELAEVNAELVDLTSLIGSPAYRAGSPQREALDARIAALAARQE | |
| | ELEGLEARPSGWEWRETGQRFGDWWREQDTAAKNTWLRSMNVRLTFDVR | |
| | GGLTRTIDFGDLQEYEQHLRLGSVVERLHTGMSGSGKDKKSDGKDSQKK* | |
| A315R | MPKKKRKVQFELADPYTQPRFAAAGSGKDKKSDGKDSQKKGSRALVVIRLS | SEQ ID |
| Stabi- | RVTDATTSPERQLESCQQLCAQRGWDVVGVAEDLDVSGAVDPFDRKRRPNL | NO: 720 |
| lized-7 | ARWLAFEEQPFDVIVAYRVDRLTRSIRHLQQLVHWAEDHKKLVVSATEAHF | |
| | DTTTPFAAVVIALMGTVAQMELEAIKERNRSAAHFNIRAGKYRGSLPPWGYL | |
| | PTRVDGEWRLVPDPVQRERILEVYHRVVDNHEPLHLVAHDLNRRGVLSPKD | |
| | YFAQLQGREPQGREWSATALKRSMISEAMLGYATLNGKTVRDDDGAPLVR | |
| | AEPILTREQLEALRAELVKTSRAKPAVSTPSLLLRVLFCAVCGEPAYKFRGGG | |
| | RKHPRYRCRSMGFPKHCGNGTVAMAEWDAFCEEQVLDLLGDAERLEKVW | |
| | VAGSDSAVELAEVNAELVDLTSLIGSPAYRAGSPQREALDARIAALAARQEE | |
| | LEGLEARPSGWEWRETGQRFGDWWREQDTAAKNTWLRSMNVRLTFDVRG | |
| | GLTRTIDFGDLQEYEQHLRLGSVVERLHTGMSGSGKDKKSDGKDSQKK* | |

Table 31 below, related to FIGS. 64C-64E. DNA primers and probes for in vivo ddPCR assay.

TABLE 31

| | | SEQ |
|---|---|---|
| Name | Sequence | ID Nos. |
| mF9 attB attL | GGAGTCCAACCTTAAGTCTC | 706 |
| FWD primer | TTG | |
| mF9 attB attL | GGTTCAGGATCTTGTTGGCG | 707 |
| REV primer | | |
| mF9 attB attL | FAM-CTTGTCGACGACGGCG | 697 |
| probe | GTCTCAGTG | |
| mF9 attB attR | GCAACTTGGGCTACCAGAT | 708 |
| FWD primer | | |
| mF9 attB attR | GCTGTTTTGTTTCCACTGTG | 709 |
| REV primer | TAC | |
| mF9 attB attR | FAM-CGGTCTCCGTCGTCAG | 710 |
| probe | GATCAT | |
| Roas26 attB attL | AGTATACCAGCTAGCAGGCC | 711 |
| FWD primer | | |
| Rosa26 attB attL | GGTTCAGGATCTTGTTGGCG | 707 |
| REV primer | | |
| Rosa26 attB attL | FAM-CTTGTCGACGACGGC | 697 |
| probe | GGTCTCAGTG | |
| Rosa26 attB attR | GCAACTTGGGCTACCAGAT | 708 |
| FWD primer | | |
| Rosa26 attB attR | GCAGAGAACTCCCAGAAAG | 712 |
| REV primer | GT | |
| Rosa26 attB attR | FAM-CGGTCTCCGTCGTCA | 710 |
| probe | GGATCAT | |
| mTfrc probe | Applied Biosciences | |
| | cat 4458370 | |

### 7.25.7. References

1. Smith, M. C. M. (2015) Phage-encoded Serine Integrases and Other Large SerineRecombinases. Microbiol Spectr, 3.
2. Matreyek, K. A., Stephany, J. J. and Fowler, D. M. (2017) A platform for functional assessment of large variant libraries in mammalian cells. Nucleic Acids Res, 45, e102.
3. Blanch-Asensio, A., Grandela, C., Brandao, K. O., de Korte, T., Mei, H., Ariyurek, Y., Yiangou, L., Mol, M. P. H., van Meer, B. J., Kloet, S. L. et al. (2022) STRAIGHT-IN enables high-throughput targeting of large DNA payloads in human pluripotent stem cells. Cell Rep Methods, 2, 100300.
4. Low, B. E., Hosur, V., Lesbirel, S. and Wiles, M. V. (2022) Efficient targeted transgenesis of large donor DNA into multiple mouse genetic backgrounds using bacteriophage Bxb1 integrase. Sci Rep, 12, 5424.
5. Zhu, F., Gamboa, M., Farruggio, A. P., Hippenmeyer, S., Tasic, B., Schule, B., Chen-Tsai, Y. and Calos, M. P. (2014) DICE, an efficient system for iterative genomic editing in human pluripotent stem cells. Nucleic Acids Res, 42, e34.
6. Yarnall, M. T. N., Ioannidi, E. I., Schmitt-Ulms, C., Krajeski, R. N., Lim, J., Villiger, L., Zhou, W., Jiang, K., Garushyants, S. K., Roberts, N. et al. (2023) Drag-and-drop genome insertion of large sequences without double-strand DNA cleavage using CRISPR-directed integrases. Nat Biotechnol, 41, 500-512.
7. Pandey, S., Gao, X. D., Krasnow, N. A., McElroy, A., Tao, Y. A., Duby, J. E., Steinbeck, B. J., McCreary, J., Pierce, S. E., Tolar, J. et al. (2024) Efficient site-specific integration of large genes in mammalian cells via continuously evolved recombinases and prime editing. Nat Biomed Eng.
8. Anzalone, A. V., Gao, X. D., Podracky, C. J., Nelson, A. T., Koblan, L. W., Raguram, A., Levy, J. M., Mercer, J. A. M. and Liu, D. R. (2022) Programmable deletion, replacement, integration and inversion of large DNA sequences with twin prime editing. Nat Biotechnol, 40, 731-740.
9. Durrant, M. G., Fanton, A., Tycko, J., Hinks, M., Chandrasekaran, S. S., Perry, N. T., Schaepe, J., Du, P. P., Lotfy, P., Bassik, M. C. et al. (2023) Systematic discovery of recombinases for efficient integration of large DNA sequences into the human genome. Nat Biotechnol, 41, 488-499.
10. Rutherford, K., Yuan, P., Perry, K., Sharp, R. and Van Duyne, G. D. (2013) Attachment site recognition and regulation of directionality by the serine integrases. Nucleic Acids Res, 41, 8341-8356.
11. Rutherford, K. and Van Duyne, G. D. (2014) The ins and outs of serine integrase site specific recombination. Curr Opin Struct Biol, 24, 125-131.
12. Xu, Z., Thomas, L., Davies, B., Chalmers, R., Smith, M. and Brown, W. (2013) Accuracy and efficiency define Bxb1 integrase as the best of fifteen candidate serine recombinases for the integration of DNA into the human genome. BMC Biotechnol, 13, 87.
13. Hew, B. E., Gupta, S., Sato, R., Waller, D. F., Stoytchev, I., Short, J. E., Sharek, L., Tran, C. T., Badran, A. H. and Owens, J. B. (2024) Directed evolution of hyperactive integrases for site specific insertion of transgenes. Nucleic Acids Res, 52, e64.
14. Merrick, C. A., Zhao, J. and Rosser, S. J. (2018) Serine Integrases: Advancing Synthetic Biology. ACS Synth Biol, 7, 299-310.
15. Hazelbaker, D. Z., Mehta, J. B., McGinnis, C., Santesmasses, D., Bara, A. M., Liang, X., Biondi, T., Fennell, T., Homer, N., Estes, B. et al. (2024) Large Serine Integrase Off-target Discovery and Validation for Therapeutic Genome Editing. bioRxiv, 2024.2008.2023.609471.
16. Jusiak, B., Jagtap, K., Gaidukov, L., Duportet, X., Bandara, K., Chu, J., Zhang, L., Weiss, R. and Lu, T. K. (2019) Comparison of Integrases Identifies Bxb1-GA Mutant as the Most Efficient Site-Specific Integrase System in Mammalian Cells. ACS Synth Biol, 8, 16-24.
17. Kim, A. I., Ghosh, P., Aaron, M. A., Bibb, L. A., Jain, S. and Hatfull, G. F. (2003) Mycobacteriophage Bxb1 integrates into the Mycobacterium smegmatis groEL1 gene. Mol Microbiol, 50, 463-473.
18. Ghosh, P., Pannunzio, N. R. and Hatfull, G. F. (2005) Synapsis in phage Bxb1 integration: selection mechanism for the correct pair of recombination sites. J Mol Biol, 349, 331-348.
19. Bai, H., Sun, M., Ghosh, P., Hatfull, G. F., Grindley, N. D. and Marko, J. F. (2011) Single-molecule analysis reveals the molecular bearing mechanism of DNA strand exchange by a serine recombinase. Proc Natl Acad Sci USA, 108, 7419-7424.
20. Ghosh, P., Wasil, L. R. and Hatfull, G. F. (2006) Control of phage Bxb1 excision by a novel recombination directionality factor. PLoS Biol, 4, e186.
21. Li, H., Sharp, R., Rutherford, K., Gupta, K. and Van Duyne, G. D. (2018) Serine Integrase attP Binding and Specificity. J Mol Biol, 430, 4401-4418.
22. Rethi-Nagy, Z., Abraham, E., Udvardy, K., Klement, E., Darula, Z., Pal, M., Katona, R. L., Tubak, V., Pali, T., Kota, Z. et al. (2022) STABILON, a Novel Sequence Motif That Enhances the Expression and Accumulation of Intracellular and Secreted Proteins. Int J Mol Sci, 23.
23. Zhu, Y., Saribas, A. S., Liu, J., Lin, Y., Bodnar, B., Zhao, R., Guo, Q., Ting, J., Wei, Z., Ellis, A. et al. (2023) Protein expression/secretion boost by a novel unique 21-mer cis-regulatory motif (Exin21) via mRNA stabilization. Mol Ther, 31, 1136-1158.

### 8. SEQUENCES

TABLE 26
Additional Sequences

| SEQ | | |
|---|---|---|
| ID | | |
| NO: | Name | Sequence |
| 615 | PL758 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLWVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKVGGSVSGW |
| | | RLFKKIS* |
| 616 | PL758_ | ATGAAGAGAACCGCCGACGGCAGCGAGTTCGAGAGCCCCAAGAAGAAGAGAAAGGTGGA |
| | DNA | CAAGAAGTACAGCATCGGCCTGGACATCGGCACCAACAGCGTGGGCTGGGCCGTGATCA |
| | | CCGACGAGTACAAGGTGCCCAGCAAGAAGTTCAAGGTGCTGGGCAACACCGACAGACAC |
| | | AGCATCAAGAAGAACCTGATCGGCGCCCTGCTGTTCGACAGCGGCGAGACCGCCGAGGC |
| | | CACCAGACTGAAGAGAACCGCCAGAAGAAGATACACCAGAAGAAAGAACAGAATCTGCT |
| | | ACCTGCAGGAGATCTTCAGCAACGAGATGGCCAAGGTGGACGACAGCTTCTTCCACAGA |
| | | CTGGAGGAGAGCTTCCTGGTGGAGGAGGACAAGAAGCACGAGAGACACCCCATCTTCGG |
| | | CAACATCGTGGACGAGGTGGCCTACCACGAGAAGTACCCCACCATCTACCACCTGAGAA |
| | | AGAAGCTGGTGGACAGCACCGACAAGGCCGACCTGAGACTGATCTACCTGGCCCTGGCC |
| | | CACATGATCAAGTTCAGAGGCCACTTCCTGATCGAGGGCGACCTGAACCCCGACAACAG |
| | | CGACGTGGACAAGCTGTTCATCCAGCTGGTGCAGACCTACAACCAGCTGTTCGAGGAGA |
| | | ACCCCATCAACGCCAGCGGCGTGGACGCCAAGGCCATCCTGAGCGCCAGACTGAGCAAG |
| | | AGCAGAAGACTGGAGAACCTGATCGCCCAGCTGCCCGGCGAGAAGAAGAACGGCCTGTT |
| | | CGGCAACCTGATCGCCCTGAGCCTGGGCCTGACCCCCAACTTCAAGAGCAACTTCGACC |
| | | TGGCCGAGGACGCCAAGCTGCAGCTGAGCAAGGACACCTACGACGACGACCTGGACAAC |
| | | CTGCTGGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCCCCCC |
| | | TGAGCGCCAGCATGATCAAGAGATACGACGAGCACCACCAGGACCTGACCCTGCTGAAG |
| | | GCCCTGGTGAGACAGCAGCTGCCCGAGAAGTACAAGGAGATCTTCTTCGACCAGAGCAA |
| | | GAACGGCTACGCCGGCTACATCGACGGCGGCGCCAGCCAGGAGGAGTTCTACAAGTTCA |
| | | TCAAGCCCATCCTGGAGAAGATGGACGGCACCGAGGAGCTGCTGGTGAAGCTGAACAGA |
| | | GAGGACCTGCTGAGAAAGCAGAGAACCTTCGACAACGGCAGCATCCCCCACCAGATCCA |
| | | CCTGGGCGAGCTGCACGCCATCCTGAGAAGACAGGAGGACTTCTACCCCTTCCTGAAGG |
| | | ACAACAGAGAGAAGATCGAGAAGATCCTGACCTTCAGAATCCCCTACTACGTGGGCCCC |
| | | CTGGCCAGAGGCAACAGCAGATTCGCCTGGATGACCAGAAAGAGCGAGGAGACCATCAC |
| | | CCCCTGGAACTTCGAGGAGGTGGTGGACAAGGGCGCCAGCGCCCAGAGCTTCATCGAGA |
| | | GAATGACCAACTTCGACAAGAACCTGCCCAACGAGAAGGTGCTGCCCAAGCACAGCCTG |
| | | CTGTACGAGTACTTCACCGTGTACAACGAGCTGACCAAGGTGAAGTACGTGACCGAGGG |
| | | CATGAGAAAGCCCGCCTTCCTGAGCGGCGAGCAGAAGAAGGCCATCGTGGACCTGCTGT |
| | | TCAAGACCAACAGAAAGGTGACCGTGAAGCAGCTGAAGGAGGACTACTTCAAGAAGATC |
| | | GAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGA |
| | | ACGAGGACATCCTGGAGGACATCGTGCTGACCCTGACCCTGTTCGAGGACAGAGAGATG |
| | | ATCGAGGAGAGACTGAAGACCTACGCCCACCTGTTCGACGACAAGGTGATGAAGCAGCT |
| | | GAAGAGAAGAAGATACACCGGCTGGGGCAGACTGAGCAGAAAGCTGATCAACGGCATCA |
| | | GAGACAAGCAGAGCGGCAAGACCATCCTGGACTTCCTGAAGAGTGACGGCTTCGCCAAC |
| | | AGAAACTTCATGCAGCTGATCCACGACGACAGCCTGACCTTCAAGGAGGACATCCAGAA |
| | | GGCCCAGGTGAGCGGCCAGGGCGACAGCCTGCACGAGCACATCGCCAACCTGGCCGGCA |
| | | GCCCCGCCATCAAGAAGGGCATCCTGCAGACCGTGAAGGTGGTGGACGAGCTGGTGAAG |
| | | GTGATGGGCAGACACAAGCCCGAGAACATCGTGATCGAGATGGCCAGAGAGAACCAGAC |
| | | CACCCAGAAGGGCCAGAAGAACAGCAGAGAGAGAATGAAGAGAATCGAGGAGGGCATCA |
| | | AGGAGCTGGGCAGCCAGATCCTGAAGGAGCACCCCGTGGAGAACACCCAGCTGCAGAAC |
| | | GAGAAGCTGTACCTGTACTACCTGCAGAACGGCAGAGACATGTACGTGGACCAGGAGCT |
| | | GGACATCAACAGACTGAGCGACTACGACGTGGACGCCATCGTGCCCCAGAGCTTCCTGA |
| | | AGGACGACAGCATCGACAACAAGGTGCTGACCAGAAGCGACAAGAACAGAGGCAAGAGC |
| | | GACAACGTGCCCAGCGAGGAGGTGGTGAAGAAGATGAAGAACTACTGGAGACAGCTGCT |
| | | GAACGCCAAGCTGATCACCCAGAGAAAGTTCGACAACCTGACCAAGGCCGAGAGAGGCG |
| | | GCCTGAGCGAGCTGGACAAGGCCGGCTTCATCAAGAGACAGCTGGTGGAGACCAGACAG |
| | | ATCACCAAGCACGTGGCCCAGATCCTGGACAGCAGAATGAACACCAAGTACGACGAGAA |
| | | CGACAAGCTGATCAGAGAGGTGAAGGTGATCACCCTGAAGAGTAAGCTGGTGAGCGACT |
| | | TCAGAAAGGACTTCCAGTTCTACAAGGTGAGAGAGATCAACAACTACCACCACGCCCAC |
| | | GACGCCTACCTGAACGCCGTGGTGGGCACCGCCCTGATCAAGAAGTACCCCAAGCTGGA |
| | | GAGCGAGTTCGTGTACGGCGACTACAAGGTGTACGACGTGAGAAAGATGATCGCCAAGA |
| | | GCGAGCAGGAGATCGGCAAGGCCACCGCCAAGTACTTCTTCTACAGCAACATCATGAAC |
| | | TTCTTCAAGACCGAGATCACCCTGGCCAACGGCGAGATCAGAAAGAGACCCCTGATCGA |
| | | GACCAACGGCGAGACCGGCGAGATCGTGTGGGACAAGGGCAGAGACTTCGCCACCGTGA |
| | | GAAAGGTGCTGAGCATGCCCCAGGTGAACATCGTGAAGAAGACCGAGGTGCAGACCGGC |
| | | GGCTTCAGCAAGGAGAGCATCCTGCCCAAGAGAAACAGCGACAAGCTGATCGCCAGAAA |
| | | GAAGGACTGGGACCCCAAGAAGTACGGCGGCTTCGACAGCCCCACCGTGGCCTACAGCG |
| | | TGCTGGTGGTGGCCAAGGTGGAGAAGGGCAAGAGCAAGAAGCTGAAGAGTGTGAAGGAG |
| | | CTGCTGGGCATCACCATCATGGAGAGAAGCAGCTTCGAGAAGAACCCCATCGACTTCCT |
| | | GGAGGCCAAGGGCTACAAGGAGGTGAAGAAGGACCTGATCATCAAGCTGCCCAAGTACA |
| | | GCCTGTTCGAGCTGGAGAACGGCAGAAAGAGAATGCTGGCCAGCGCCGGCGAGCTGCAG |
| | | AAGGGCAACGAGCTGGCCCTGCCCAGCAAGTACGTGAACTTCCTGTACCTGGCCAGCCA |
| | | CTACGAGAAGCTGAAGGGCAGCCCCGAGGACAACGAGCAGAAGCAGCTGTTCGTGGAGC |
| | | AGCACAAGCACTACCTGGACGAGATCATCGAGCAGATCAGCGAGTTCAGCAAGAGAGTG |
| | | ATCCTGGCCGACGCCAACCTGGACAAGGTGCTGAGCGCCTACAACAAGCACAGAGACAA |
| | | GCCCATCAGAGAGCAGGCCGAGAACATCATCCACCTGTTCACCCTGACCAACCTGGGCG |
| | | CCCCCGCCGCCTTCAAGTACTTCGACACCACCATCGACAGAAAGAGATACACCAGCACC |
| | | AAGGAGGTGCTGGACGCCACCCTGATCCACCAGAGCATCACCGGCCTGTACGAGACCAG |
| | | AATCGACCTGAGCCAGCTGGGGGGCGACAGCGGCGGCAGCAGCGGCGGCAGCAGCGGCA |
| | | GCGAGACCCCCGGCACCAGCGAGAGCGCCACCCCCGAGAGCAGCGGCGGCAGCAGCGGC |
| | | GGCAGCAGCACCCTGAACATCGAGGACGAGTACAGACTGCACGAGACCAGCAAGGAGCC |
| | | CGACGTGAGCCTGGGCAGCACCTGGCTGAGCGACTTCCCCCAGGCCTGGGCCGAGACCG |
| | | GCGGCATGGGCCTGGCCGTGAGACAGGCCCCCCTGATCATCCCCCTGAAGGCCACCAGC |
| | | ACCCCCGTGAGCATCAAGCAGTACCCCATGAGCCAGGAGGCCAGACTGGGCATCAAGCC |
| | | CCACATCCAGAGACTGCTGGACCAGGGCATCCTGGTGCCCTGCCAGAGCCCCTGGAACA |
| | | CCCCCCTGCTGCCCGTGAAGAAGCCCGGCACCAACGACTACAGACCCGTGCAGGACCTG |
| | | AGAGAGGTGAACAAGAGAGTGGAGGACATCCACCCCACCGTGCCCAACCCCTACAACCT |
| | | GCTGAGCGGCCTGCCCCCCAGCCACCAGTGGTACACCGTGCTGGACCTGAAGGACGCCT |
| | | TCTTCTGCCTGAGACTGCACCCCACCAGCCAGCCCCTGTTCGCCTTCGAGTGGAGAGAC |
| | | CCCGAGATGGGCATCAGCGGCCAGCTGACCTGGACCAGACTGCCCCAGGGCTTCAAGAA |
| | | CAGCCCCACCCTGTTCAACGAGGCCCTGCACAGAGACCTGGCCGACTTCAGAATCCAGC |
| | | ACCCCGACCTGATCCTGCTGCAGTACGTGGACGACCTGCTGCTGGCCGCCACCAGCGAG |
| | | CTGGACTGCCAGCAGGGCACCAGAGCCCTGCTGCAGACCCTGGGCAACCTGGGCTACAG |
| | | AGCCAGCGCCAAGAAGGCCCAGATCTGCCAGAAGCAGGTGAAGTACCTGGGCTACCTGC |
| | | TGAAGGAGGGCCAGAGATGGCTGACCGAGGCCAGAAAGGAGACCGTGATGGGCCAGCCC |
| | | ACCCCCAAGACCCCCAGACAGCTGAGAGAGTTCCTGGGCAAGGCCGGCTTCTGCAGACT |
| | | GTTCATCCCCGGCTTCGCCGAGATGGCCGCCCCCCTGTACCCCCTGACCAAGCCCGGCA |
| | | CCCTGTTCAACTGGGGCCCCGACCAGCAGAAGGCCTACCAGGAGATCAAGCAGGCCCTG |
| | | CTGACCGCCCCCGCCCTGGGCCTGCCCGACCTGACCAAGCCCTTCGAGCTGTTCGTGGA |
| | | CGAGAAGCAGGGCTACGCCAAGGGCGTGCTGACCCAGAAGCTGGGCCCCTGGAGAAGAC |
| | | CCGTGGCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATGGGCCA |
| | | GCCCCTGGTGATCCTGGCCCCCCACGCCGTGGAGGCCCTGGTGAAGCAGCCCCCCGACA |
| | | GATGGCTGAGCAACGCCAGAATGACCCACTACCAGGCCCTGCTGCTGGACACCGACAGA |
| | | GTGCAGTTCGGCCCCGTGGTGGCCCTGAACCCCGCCACCCTGCTGCCCCTGCCCGAGGA |
| | | GGGCCTGCAGCACAACTGCCTGGACATCCTGGCCGAGGCCCACGGCACCAGACCCGACC |
| | | TGACCGACCAGCCCCTGCCCGACGCCGACCACACCTGGTACACCGACGGCAGCAGCCTG |
| | | CTGCAGGAGGGCCAGAGAAAGGCCGGCGCCGCCGTGACCACCGAGACCGAGGTGATCTG |
| | | GGCCAAGGCCCTGCCCGCCGGCACCAGCGCCCAGAGAGCCGAGCTGATCGCCCTGACCC |
| | | AGGCCCTGAAGATGGCCGAGGGCAAGAAGCTGAACGTGTACACCGACAGCAGATACGCC |
| | | TTCGCCACCGCCCACATCCACGGCGAGATCTACAGAAGAAGAGGCTGGCTGACCAGCGA |
| | | GGGCAAGGAGATCAAGAACAAGGACGAGATCCTGGCCCTGCTGAAGGCCCTGTTCCTGC |
| | | CCAAGAGACTGAGCATCATCCACTGCCCCGGCCACCAGAAGGGCCACAGCGCCGAGGCC |
| | | AGAGGCAACAGAATGGCCGACCAGGCCGCCAGAAAGGCCGCCATCACCGAGACCCCCGA |
| | | CACCAGCACCCTGCTGATCGAGAACAGCAGCCCCAGCGGCGGCAGCAAGAGAACCGCCG |
| | | ACGGCAGCGAGTTCGAGCCCAAGAAGAAGAGAAAGGTGGGCGGCAGCGTGAGCGGCTGG |
| | | AGACTGTTCAAGAAGATCAGCTGA |
| 617 | PL883 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLWVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKV* |
| 618 | PL1323 | MPAAKRVKLDGGSRALWIRLSRVTDATTSPERQLESCQQLCAQRGWDVVGVAEDLDVSG |
| | _AA | AVDPFDRKRRPNLARWLAFEEQPFDVIVAYRVDRLTRSIRHLQQLVHWAEDHKKLVVSA |
| | | TEAHFDTTTPFAAVVIALMGTVAQMELEAIKERNRSAAHFNIRAGKYRGSLPPWGYLPT |
| | | RVDGEWRLVPDPVQRERILEVYHRVVDNHEPLHLVAHDLNRRGVLSPKDYFAQLQGREP |
| | | QGREWSATALKRSMISEAMLGYATLNGKTVRDDDGAPLVRAEPILTREQLEALRAELVK |
| | | TSRAKPAVSTPSLLLRVLFCAVCGEPAYKFAGGGRKHPRYRCRSMGFPKHCGNGTVAMA |
| | | EWDAFCEEQVLDLLGDAERLEKVWVAGSDSAVELAEVNAELVDLTSLIGSPAYRAGSPQ |
| | | REALDARIAALAARQEELEGLEARPSGWEWRETGQRFGDWWREQDTAAKNTWLRSMNVR |
| | | LTFDVRGGLTRTIDFGDLQEYEQHLRLGSVVERLHTGMSGGSPKKKRKV* |
| 619 | PL1323_ | ATGcctgctgctaagagagtgaaactggatggcggcagcAGAGCCCTGGTGGTGATCAG |
| | DNA | ACTGAGCAGAGTGACCGACGCCACCACCAGCCCCGAGAGACAGCTGGAGAGCTGCCAGC |
| | | AGCTGTGCGCCCAGAGAGGCTGGGACGTGGTGGGCGTGGCCGAGGACCTGGACGTGAGC |
| | | GGCGCCGTGGACCCCTTCGACAGAAAGAGAAGACCCAACCTGGCCAGATGGCTGGCCTT |
| | | CGAGGAGCAGCCCTTCGACGTGATCGTGGCCTACAGAGTGGACAGACTGACCAGAAGCA |
| | | TCAGACACCTGCAGCAGCTGGTGCACTGGGCCGAGGACCACAAGAAGCTGGTGGTGAGC |
| | | GCCACCGAGGCCCACTTCGACACCACCACCCCCTTCGCCGCCGTGGTGATCGCCCTGAT |
| | | GGGCACCGTGGCCCAGATGGAGCTGGAGGCCATCAAGGAGAGAAACAGAAGCGCCGCCC |
| | | ACTTCAACATCAGAGCCGGCAAGTACAGAGGCAGCCTGCCCCCCTGGGGCTACCTGCCC |
| | | ACCAGAGTGGACGGCGAGTGGAGACTGGTGCCCGACCCCGTGCAGAGAGAGAGAATCCT |
| | | GGAGGTGTACCACAGAGTGGTGGACAACCACGAGCCCCTGCACCTGGTGGCCCACGACC |
| | | TGAACAGAAGAGGCGTGCTGAGCCCCAAGGACTACTTCGCCCAGCTGCAGGGCAGAGAG |
| | | CCCCAGGGCAGAGAGTGGAGCGCCACCGCCCTGAAGAGAAGCATGATCAGCGAGGCCAT |
| | | GCTGGGCTACGCCACCCTGAACGGCAAGACCGTGAGAGACGACGACGGCGCCCCCCTGG |
| | | TGAGAGCCGAGCCCATCCTGACCAGAGAGCAGCTGGAGGCCCTGAGAGCCGAGCTGGTG |
| | | AAGACCAGCAGAGCCAAGCCCGCCGTGAGCACCCCCAGCCTGCTGCTGAGAGTGCTGTT |
| | | CTGCGCCGTGTGCGGCGAGCCCGCCTACAAGTTCGCCGGCGGCGGCAGAAAGCACCCCA |
| | | GATACAGATGCAGAAGCATGGGCTTCCCCAAGCACTGCGGCAACGGCACCGTGGCCATG |
| | | GCCGAGTGGGACGCCTTCTGCGAGGAGCAGGTGCTGGACCTGCTGGGCGACGCCGAGAG |
| | | ACTGGAGAAGGTGTGGGTGGCCGGCAGCGACAGCGCCGTGGAGCTGGCCGAGGTGAACG |
| | | CCGAGCTGGTGGACCTGACCAGCCTGATCGGCAGCCCCGCCTACAGAGCCGGCAGCCCC |
| | | CAGAGAGAGGCCCTGGACGCCAGAATCGCCGCCCTGGCCGCCAGACAGGAGGAGCTGGA |
| | | GGGCCTGGAGGCCAGACCCAGCGGCTGGGAGTGGAGAGAGACCGGCCAGAGATTCGGCG |
| | | ACTGGTGGAGAGAGCAGGACACCGCCGCCAAGAACACCTGGCTGAGAAGCATGAACGTG |
| | | AGACTGACCTTCGACGTGAGAGGCGGCCTGACCAGAACCATCGACTTCGGCGACCTGCA |
| | | GGAGTACGAGCAGCACCTGAGACTGGGCAGCGTGGTGGAGAGACTGCACACCGGCATGA |
| | | GCGGCGGCAGCCCCAAGAAGAAGAGAAAGGTGTGA |
| 620 | PL1324_ | tcgcgcgtttcggtgatgacggtgaaaacctctgacacatgcagctcccggagacggtc |
| | DNA | acagcttgtctgtaagcggatgccgggagcagacaagcccgtcagggcgcgtcagcggg |
| | | tgttggcgggtgtcggggctggcttaactatgcggcatcagagcagattgtactgagag |
| | | tgcaccatatgcggtgtgaaataccgcacagatgcgtaaggagaaaataccgcatcagg |
| | | cgccattcgccattcaggctgcgcaactgttgggaagggcgatcggtgcgggcctcttc |
| | | gctattacgccagctggcgaaagggggatgtgctgcaaggcgattaagttgggtaacgc |
| | | cagggttttcccagtcacgacgttgtaaaacgacggccagagaattcgagctcggtacc |
| | | TAATACGACTCACTATAaGGAAGCTCAGAATAAACGCTCAACTTTGGCCGGATCTGCCA |
| | | CCATGCCCAAGAAGAAGAGAAAGGTGTACCCCTACGACGTGCCCGACTACGCCGGCAGC |
| | | AGAGCCCTGGTGGTGATCAGACTGAGCAGAGTGACCGACGCCACCACCAGCCCCGAGAG |
| | | ACAGCTGGAGAGCTGCCAGCAGCTGTGCGCCCAGAGAGGCTGGGACGTGGTGGGCGTGG |
| | | CCGAGGACCTGGACGTGAGCGGCGCCGTGGACCCCTTCGACAGAAAGAGAAGACCCAAC |
| | | CTGGCCAGATGGCTGGCCTTCGAGGAGCAGCCCTTCGACGTGATCGTGGCCTACAGAGT |
| | | GGACAGACTGACCAGAAGCATCAGACACCTGCAGCAGCTGGTGCACTGGGCCGAGGACC |
| | | ACAAGAAGCTGGTGGTGAGCGCCACCGAGGCCCACTTCGACACCACCACCCCCTTCGCC |
| | | GCCGTGGTGATCGCCCTGATGGGCACCGTGGCCCAGATGGAGCTGGAGGCCATCAAGGA |
| | | GAGAAACAGAAGCGCCGCCCACTTCAACATCAGAGCCGGCAAGTACAGAGGCAGCCTGC |
| | | CCCCCTGGGGCTACCTGCCCACCAGAGTGGACGGCGAGTGGAGACTGGTGCCCGACCCC |
| | | GTGCAGAGAGAGAGAATCCTGGAGGTGTACCACAGAGTGGTGGACAACCACGAGCCCCT |
| | | GCACCTGGTGGCCCACGACCTGAACAGAAGAGGCGTGCTGAGCCCCAAGGACTACTTCG |
| | | CCCAGCTGCAGGGCAGAGAGCCCCAGGGCAGAGAGTGGAGCGCCACCGCCCTGAAGAGA |
| | | AGCATGATCAGCGAGGCCATGCTGGGCTACGCCACCCTGAACGGCAAGACCGTGAGAGA |
| | | CGACGACGGCGCCCCCCTGGTGAGAGCCGAGCCCATCCTGACCAGAGAGCAGGTGGAGG |
| | | CCCTGAGAGCCGAGCTGGTGAAGACCAGCAGAGCCAAGCCCGCCGTGAGCACCCCCAGC |
| | | CTGCTGCTGAGAGTGCTGTTCTGCGCCGTGTGCGGCGAGCCCGCCTACAAGTTCGCCGG |
| | | CGGCGGCAGAAAGCACCCCAGATACAGATGCAGAAGCATGGGCTTCCCCAAGCACTGCG |
| | | GCAACGGCACCGTGGCCATGGCCGAGTGGGACGCCTTCTGCGAGGAGCAGGTGCTGGAC |
| | | CTGCTGGGCGACGCCGAGAGACTGGAGAAGGTGTGGGTGGCCGGCAGCGACAGCGCCGT |
| | | GGAGCTGGCCGAGGTGAACGCCGAGCTGGTGGACCTGACCAGCCTGATCGGCAGCCCCG |
| | | CCTACAGAGCCGGCAGCCCCCAGAGAGAGGCCCTGGACGCCAGAATCGCCGCCCTGGCC |
| | | GCCAGACAGGAGGAGCTGGAGGGCCTGGAGGCCAGACCCAGCGGCTGGGAGTGGAGAGA |
| | | GACCGGCCAGAGATTCGGCGACTGGTGGAGAGAGCAGGACACCGCCGCCAAGAACACCT |
| | | GGCTGAGAAGCATGAACGTGAGACTGACCTTCGACGTGAGAGGCGGCCTGACCAGAACC |
| | | ATCGACTTCGGCGACCTGCAGGAGTACGAGCAGCACCTGAGACTGGGCAGCGTGGTGGA |
| | | GAGACTGCACACCGGCATGAGCTGATGAACCAGCCTCAAGAACACCCGAATGGAGTCTC |
| | | TAAGCTACATAATACCAACTTACACTTTACAAAATGTTGTCCCCCAAAATGTAGCCATT |
| | | CGTATCTGCTCCTAATAAAAAGAAAGTTTCTTCACATTCTAAAAAAAAAAAAAAAAAAA |
| | | AAAAAAAAAAAAAAAAAAAAAAAAAAAAAAAAAAAAAAAAAAAAAAAAAAAAAAAAAAA |
| | | AAtgaagagcaggcctgcatgcaagcttctagcataaccccttggggcctctaaacggg |
| | | tcttgaggggttttttgggtgtaatcatggtcatagctgtttcctgtgtgaacagcctg |
| | | gggctcaccttcggggggcctttctgcgggtgcctaatgagtgagctaactcacattaa |
| | | ttgcgttgcgctcactgcccgctttccagtcgggaaacctgtcgtgccagctgcattaa |
| | | tgaatcggccaacgcgcggggagaggcggtttgcgtattgggcgccgaagaaaggccca |
| | | cccgtgaaggtgagcctcttccgcttcctcgctcactgactcgctgcgctcggtcgttc |
| | | ggctgcggcgagcggtatcagctcactcaaaggcggtaatacggttatccacagaatca |
| | | ggggataacgcaggaaagaacatgtgagcaaaaggccagcaaaaggccaggaaccgtaa |
| | | aaaggccgcgttgctggcgtttttccataggctccgcccccctgacgagcatcacaaaa |
| | | atcgacgctcaagtcagaggtggcgaaacccgacaggactataaagataccaggcgttt |
| | | ccccctggaagctccctcgtgcgctctcctgttccgaccctgccgcttaccggatacct |
| | | gtccgcctttctcccttcgggaagcgtggcgctttctcatagctcacgctgtaggtatc |
| | | tcagttcggtgtaggtcgttcgctccaagctgggctgtgtgcacgaaccccccgttcag |
| | | cccgaccgctgcgccttatccggtaactatcgtcttgagtccaacccggtaagacacga |
| | | cttatcgccactggcagcagccactggtaacaggattagcagagcgaggtatgtaggcg |
| | | gtgctacagagttcttgaagtggtggcctaactacggctacactagaagaacagtattt |
| | | ggtatctgcgctctgctgaagccagttaccttcggaaaaagagttggtagctcttgatc |
| | | cggcaaacaaaccaccgctggtagcggtggtttttttgtttgcaagcagcagattacgc |
| | | gcagaaaaaaaggatctcaagaagatcctttgatcttttctacggggtctgacgctcag |
| | | tggaacgaaaactcacgttaagggattttggtcatgagattatcaaaaaggatcttcac |
| | | ctagatccttttaaattaaaaatgaagttttaaatcaagcccaatctgaataatgttac |
| | | aaccaattaaccaattctgattagaaaaactcatcgagcatcaaatgaaactgcaattt |
| | | attcatatcaggattatcaataccatatttttgaaaaagccgtttctgtaatgaaggag |
| | | aaaactcaccgaggcagttccataggatggcaagatcctggtatcggtctgcgattccg |
| | | actcgtccaacatcaatacaacctattaatttcccctcgtcaaaaataaggttatcaag |
| | | tgagaaatcaccatgagtgacgactgaatccggtgagaatggcaaaagtttatgcattt |
| | | ctttccagacttgttcaacaggccagccattacgctcgtcatcaaaatcactcgcatca |
| | | accaaaccgttattcattcgtgattgcgcctgagcgagacgaaatacgcgatcgctgtt |
| | | aaaaggacaattacaaacaggaatcgaatgcaaccggcgcaggaacactgccagcgcat |
| | | caacaatattttcacctgaatcaggatattcttctaatacctggaatgctgtttttccg |
| | | gggatcgcagtggtgagtaaccatgcatcatcaggagtacggataaaatgcttgatggt |
| | | cggaagaggcataaattccgtcagccagtttagtctgaccatctcatctgtaacatcat |
| | | tggcaacgctacctttgccatgtttcagaaacaactctggcgcatcgggcttcccatac |
| | | aagcgatagattgtcgcacctgattgcccgacattatcgcgagcccatttatacccata |
| | | taaatcagcatccatgttggaatttaatcgcggcctcgacgtttcccgttgaatatggc |
| | | tcataacaccccttgtattactgtttatgtaagcagacagttttattgttcatgatgat |
| | | atatttttatcttgtgcaatgtaacatcagagattttgagacacgggccagagctgca |
| 621 | PL1325 | MPKKKRKVYPYDVPDYAKDKKSDGKDSQKKGSRALWIRLSRVTDATTSPERQLESCQQL |
| | _AA | CAQRGWDVVGVAEDLDVSGAVDPFDRKRRPNLARWLAFEEQPFDVIVAYRVDRLTRSIR |
| | | HLQQLVHWAEDHKKLVVSATEAHFDTTTPFAAVVIALMGTVAQMELEAIKERNRSAAHF |
| | | NIRAGKYRGSLPPWGYLPTRVDGEWRLVPDPVQRERILEVYHRVVDNHEPLHLVAHDLN |
| | | RRGVLSPKDYFAQLQGREPQGREWSATALKRSMISEAMLGYATLNGKTVRDDDGAPLVR |
| | | AEPILTREQLEALRAELVKTSRAKPAVSTPSLLLRVLFCAVCGEPAYKFAGGGRKHPRY |
| | | RCRSMGFPKHCGNGTVAMAEWDAFCEEQVLDLLGDAERLEKVWVAGSDSAVELAEVNAE |
| | | LVDLTSLIGSPAYRAGSPQREALDARIAALAARQEELEGLEARPSGWEWRETGQRFGDW |
| | | WREQDTAAKNTWLRSMNVRLTFDVRGGLTRTIDFGDLQEYEQHLRLGSVVERLHTGMS* |
| 622 | PL1935_ | ATGCCCAAGAAGAAGAGAAAGGTGTACCCCTACGACGTGCCCGACTACGCCAAGGACAA |
| | DNA | GAAATCCGATGGCAAGGACAGCCAGAAAAAAGGCAGCAGAGCCCTGGTGGTGATCAGAC |
| | | TGAGCAGAGTGACCGACGCCACCACCAGCCCCGAGAGACAGCTGGAGAGCTGCCAGCAG |
| | | CTGTGCGCCCAGAGAGGCTGGGACGTGGTGGGCGTGGCCGAGGACCTGGACGTGAGCGG |
| | | CGCCGTGGACCCCTTCGACAGAAAGAGAAGACCCAACCTGGCCAGATGGCTGGCCTTCG |
| | | AGGAGCAGCCCTTCGACGTGATCGTGGCCTACAGAGTGGACAGACTGACCAGAAGCATC |
| | | AGACACCTGCAGCAGCTGGTGCACTGGGCCGAGGACCACAAGAAGCTGGTGGTGAGCGC |
| | | CACCGAGGCCCACTTCGACACCACCACCCCCTTCGCCGCCGTGGTGATCGCCCTGATGG |
| | | GCACCGTGGCCCAGATGGAGCTGGAGGCCATCAAGGAGAGAAACAGAAGCGCCGCCCAC |
| | | TTCAACATCAGAGCCGGCAAGTACAGAGGCAGCCTGCCCCCCTGGGGCTACCTGCCCAC |
| | | CAGAGTGGACGGCGAGTGGAGACTGGTGCCCGACCCCGTGCAGAGAGAGAGAATCCTGG |
| | | AGGTGTACCACAGAGTGGTGGACAACCACGAGCCCCTGCACCTGGTGGCCCACGACCTG |
| | | AACAGAAGAGGCGTGCTGAGCCCCAAGGACTACTTCGCCCAGCTGCAGGGCAGAGAGCC |
| | | CCAGGGCAGAGAGTGGAGCGCCACCGCCCTGAAGAGAAGCATGATCAGCGAGGCCATGC |
| | | TGGGCTACGCCACCCTGAACGGCAAGACCGTGAGAGACGACGACGGCGCCCCCCTGGTG |
| | | AGAGCCGAGCCCATCCTGACCAGAGAGCAGCTGGAGGCCCTGAGAGCCGAGCTGGTGAA |
| | | GACCAGCAGAGCCAAGCCCGCCGTGAGCACCCCCAGCCTGCTGCTGAGAGTGCTGTTCT |
| | | GCGCCGTGTGCGGCGAGCCCGCCTACAAGTTCGCCGGCGGCGGCAGAAAGCACCCCAGA |
| | | TACAGATGCAGAAGCATGGGCTTCCCCAAGCACTGCGGCAACGGCACCGTGGCCATGGC |
| | | CGAGTGGGACGCCTTCTGCGAGGAGCAGGTGCTGGACCTGCTGGGCGACGCCGAGAGAC |
| | | TGGAGAAGGTGTGGGTGGCCGGCAGCGACAGCGCCGTGGAGCTGGCCGAGGTGAACGCC |
| | | GAGCTGGTGGACCTGACCAGCCTGATCGGCAGCCCCGCCTACAGAGCCGGCAGCCCCCA |
| | | GAGAGAGGCCCTGGACGCCAGAATCGCCGCCCTGGCCGCCAGACAGGAGGAGCTGGAGG |
| | | GCCTGGAGGCCAGACCCAGCGGCTGGGAGTGGAGAGAGACCGGCCAGAGATTCGGCGAC |
| | | TGGTGGAGAGAGCAGGACACCGCCGCCAAGAACACCTGGCTGAGAAGCATGAACGTGAG |
| | | ACTGACCTTCGACGTGAGAGGCGGCCTGACCAGAACCATCGACTTCGGCGACCTGCAGG |
| | | AGTACGAGCAGCACCTGAGACTGGGCAGCGTGGTGGAGAGACTGCACACCGGCATGAGC |
| | | TGA |
| 623 | PL1383 | tcgcgcgtttcggtgatgacggtgaaaacctctgacacatgcagctcccggagacggtc |
| | | acagcttgtctgtaagcggatgccgggagcagacaagcccgtcagggcgcgtcagcggg |
| | | tgttggcgggtgtcggggctggcttaactatgcggcatcagagcagattgtactgagag |
| | | tgcaccatatgcggtgtgaaataccgcacagatgcgtaaggagaaaataccgcatcagg |
| | | cgccattcgccattcaggctgcgcaactgttgggaagggcgatcggtgcgggcctcttc |
| | | gctattacgccagctggcgaaagggggatgtgctgcaaggcgattaagttgggtaacgc |
| | | cagggttttcccagtcacgacgttgtaaaacgacggccagagaattcgagctcggtacc |
| | | TAATACGACTCACTATAGAGAATAAACTAGTATTCTTCTGGTCCCCACAGACTCAGAGA |
| | | GAACCCGCCACCATGCCCAAGAAGAAGAGAAAGGTGTACCCCTACGACGTGCCCGACTA |
| | | CGCCGGCAGCAGAGCCCTGGTGGTGATCAGACTGAGCAGAGTGACCGACGCCACCACCA |
| | | GCCCCGAGAGACAGCTGGAGAGCTGCCAGCAGCTGTGCGCCCAGAGAGGCTGGGACGTG |
| | | GTGGGCGTGGCCGAGGACCTGGACGTGAGCGGCGCCGTGGACCCCTTCGACAGAAAGAG |
| | | AAGACCCAACCTGGCCAGATGGCTGGCCTTCGAGGAGCAGCCCTTCGACGTGATCGTGG |
| | | CCTACAGAGTGGACAGACTGACCAGAAGCATCAGACACCTGCAGCAGCTGGTGCACTGG |
| | | GCCGAGGACCACAAGAAGCTGGTGGTGAGCGCCACCGAGGCCCACTTCGACACCACCAC |
| | | CCCCTTCGCCGCCGTGGTGATCGCCCTGATGGGCACCGTGGCCCAGATGGAGCTGGAGG |
| | | CCATCAAGGAGAGAAACAGAAGCGCCGCCCACTTCAACATCAGAGCCGGCAAGTACAGA |
| | | GGCAGCCTGCCCCCCTGGGGCTACCTGCCCACCAGAGTGGACGGCGAGTGGAGACTGGT |
| | | GCCCGACCCCGTGCAGAGAGAGAGAATCCTGGAGGTGTACCACAGAGTGGTGGACAACC |
| | | ACGAGCCCCTGCACCTGGTGGCCCACGACCTGAACAGAAGAGGCGTGCTGAGCCCCAAG |
| | | GACTACTTCGCCCAGCTGCAGGGCAGAGAGCCCCAGGGCAGAGAGTGGAGCGCCACCGC |
| | | CCTGAAGAGAAGCATGATCAGCGAGGCCATGCTGGGCTACGCCACCCTGAACGGCAAGA |
| | | CCGTGAGAGACGACGACGGCGCCCCCCTGGTGAGAGCCGAGCCCATCCTGACCAGAGAG |
| | | CAGCTGGAGGCCCTGAGAGCCGAGCTGGTGAAGACCAGCAGAGCCAAGCCCGCCGTGAG |
| | | CACCCCCAGCCTGCTGCTGAGAGTGCTGTTCTGCGCCGTGTGCGGCGAGCCCGCCTACA |
| | | AGTTCGCCGGCGGCGGCAGAAAGCACCCCAGATACAGATGCAGAAGCATGGGCTTCCCC |
| | | AAGCACTGCGGCAACGGCACCGTGGCCATGGCCGAGTGGGACGCCTTCTGCGAGGAGCA |
| | | GGTGCTGGACCTGCTGGGCGACGCCGAGAGACTGGAGAAGGTGTGGGTGGCCGGCAGCG |
| | | ACAGCGCCGTGGAGCTGGCCGAGGTGAACGCCGAGCTGGTGGACCTGACCAGCCTGATC |
| | | GGCAGCCCCGCCTACAGAGCCGGCAGCCCCCAGAGAGAGGCCCTGGACGCCAGAATCGC |
| | | CGCCCTGGCCGCCAGACAGGAGGAGCTGGAGGGCCTGGAGGCCAGACCCAGCGGCTGGG |
| | | AGTGGAGAGAGACCGGCCAGAGATTCGGCGACTGGTGGAGAGAGCAGGACACCGCCGCC |
| | | AAGAACACCTGGCTGAGAAGCATGAACGTGAGACTGACCTTCGACGTGAGAGGCGGCCT |
| | | GACCAGAACCATCGACTTCGGCGACCTGCAGGAGTACGAGCAGCACCTGAGACTGGGCA |
| | | GCGTGGTGGAGAGACTGCACACCGGCATGAGCTGATAACTCGAGCTGGTACTGCATGCA |
| | | CGCAATGCTAGCTGCCCCTTTCCCGTCCTGGGTACCCCGAGTCTCCCCCGACCTCGGGT |
| | | CCCAGGTATGCTCCCACCTCCACCTGCCCCACTCACCACCTCTGCTAGTTCCAGACACC |
| | | TCCCAAGCACGCAGCAATGCAGCTCAAAACGCTTAGCCTAGCCACACCCCCACGGGAAA |
| | | CAGCAGTGATTAACCTTTAGCAATAAACGAAAGTTTAACTAAGCTATACTAACCCCAGG |
| | | GTTGGTCAATTTCGTGCCAGCCACACCCTGGAGCTAGCAAAAAAAAAAAAAAAAAAAAA |
| | | AAAAAAAAAgcatatgactAAAAAAAAAAAAAAAAAAAAAAAAAAAAAAAAAAAAAAAA |
| | | AAAAAAAAAAAAAAAAAAAAAAAAAAAAAAtgaagagcaggcctgcatgcaagcttcta |
| | | gcataaccccttggggcctctaaacgggtcttgaggggttttttgggtgtaatcatggt |
| | | catagctgtttcctgtgtgaacagcctggggctcaccttcggggggcctttctgcgggt |
| | | gcctaatgagtgagctaactcacattaattgcgttgcgctcactgcccgctttccagtc |
| | | gggaaacctgtcgtgccagctgcattaatgaatcggccaacgcgcggggagaggcggtt |
| | | tgcgtattgggcgccgaagaaaggcccacccgtgaaggtgagcctcttccgcttcctcg |
| | | ctcactgactcgctgcgctcggtcgttcggctgcggcgagcggtatcagctcactcaaa |
| | | ggcggtaatacggttatccacagaatcaggggataacgcaggaaagaacatgtgagcaa |
| | | aaggccagcaaaaggccaggaaccgtaaaaaggccgcgttgctggcgtttttccatagg |
| | | ctccgcccccctgacgagcatcacaaaaatcgacgctcaagtcagaggtggcgaaaccc |
| | | gacaggactataaagataccaggcgtttccccctggaagctccctcgtgcgctctcctg |
| | | ttccgaccctgccgcttaccggatacctgtccgcctttctcccttcgggaagcgtggcg |
| | | ctttctcatagctcacgctgtaggtatctcagttcggtgtaggtcgttcgctccaagct |
| | | gggctgtgtgcacgaaccccccgttcagcccgaccgctgcgccttatccggtaactatc |
| | | gtcttgagtccaacccggtaagacacgacttatcgccactggcagcagccactggtaac |
| | | aggattagcagagcgaggtatgtaggcggtgctacagagttcttgaagtggtggcctaa |
| | | ctacggctacactagaagaacagtatttggtatctgcgctctgctgaagccagttacct |
| | | tcggaaaaagagttggtagctcttgatccggcaaacaaaccaccgctggtagcggtggt |
| | | ttttttgtttgcaagcagcagattacgcgcagaaaaaaaggatctcaagaagatccttt |
| | | gatcttttctacggggtctgacgctcagtggaacgaaaactcacgttaagggattttgg |
| | | tcatgagattatcaaaaaggatcttcacctagatccttttaaattaaaaatgaagtttt |
| | | aaatcaagcccaatctgaataatgttacaaccaattaaccaattctgattagaaaaact |
| | | catcgagcatcaaatgaaactgcaatttattcatatcaggattatcaataccatatttt |
| | | tgaaaaagccgtttctgtaatgaaggagaaaactcaccgaggcagttccataggatggc |
| | | aagatcctggtatcggtctgcgattccgactcgtccaacatcaatacaacctattaatt |
| | | tcccctcgtcaaaaataaggttatcaagtgagaaatcaccatgagtgacgactgaatcc |
| | | ggtgagaatggcaaaagtttatgcatttctttccagacttgttcaacaggccagccatt |
| | | acgctcgtcatcaaaatcactcgcatcaaccaaaccgttattcattcgtgattgcgcct |
| | | gagcgagacgaaatacgcgatcgctgttaaaaggacaattacaaacaggaatcgaatgc |
| | | aaccggcgcaggaacactgccagcgcatcaacaatattttcacctgaatcaggatattc |
| | | ttctaatacctggaatgctgtttttccggggatcgcagtggtgagtaaccatgcatcat |
| | | caggagtacggataaaatgcttgatggtcggaagaggcataaattccgtcagccagttt |
| | | agtctgaccatctcatctgtaacatcattggcaacgctacctttgccatgtttcagaaa |
| | | caactctggcgcatcgggcttcccatacaagcgatagattgtcgcacctgattgcccga |
| | | cattatcgcgagcccatttatacccatataaatcagcatccatgttggaatttaatcgc |
| | | ggcctcgacgtttcccgttgaatatggctcataacaccccttgtattactgtttatgta |
| | | agcagacagttttattgttcatgatgatatatttttatcttgtgcaatgtaacatcaga |
| | | gattttgagacacgggccagagctgca |
| 624 | PL1409 | tcgcgcgtttcggtgatgacggtgaaaacctctgacacatgcagctcccggagacggtc |
| | | acagcttgtctgtaagcggatgccgggagcagacaagcccgtcagggcgcgtcagcggg |
| | | tgttggcgggtgtcggggctggcttaactatgcggcatcagagcagattgtactgagag |
| | | tgcaccatatgcggtgtgaaataccgcacagatgcgtaaggagaaaataccgcatcagg |
| | | cgccattcgccattcaggctgcgcaactgttgggaagggcgatcggtgcgggcctcttc |
| | | gctattacgccagctggcgaaagggggatgtgctgcaaggcgattaagttgggtaacgc |
| | | cagggttttcccagtcacgacgttgtaaaacgacggccagagaattcgagctcggtacc |
| | | TAATACGACTCACTATAGAGAATAAACTAGTATTCTTCTGGTCCCCACAGACTCAGAGA |
| | | GAACCCGCCACCATGCCAAAGAAGAAGCGAAAGGTGTATCCCTACGACGTGCCCGACTA |
| | | CGCCGGTAGTCGGGCACTCGTAGTGATACGCCTTTCGCGGGTCACTGATGCAACGACGA |
| | | GTCCAGAGCGTCAGCTGGAATCCTGTCAGCAGCTATGCGCGCAGAGGGGCTGGGACGTG |
| | | GTCGGCGTGGCTGAGGATTTGGACGTCTCTGGTGCGGTCGATCCCTTCGACCGCAAGAG |
| | | ACGTCCAAACCTCGCCCGCTGGCTCGCGTTCGAGGAGCAGCCCTTCGACGTGATAGTTG |
| | | CTTACAGGGTTGACCGGCTGACGCGCTCGATTCGTCACTTGCAGCAGTTGGTCCACTGG |
| | | GCGGAGGACCACAAGAAGCTTGTGGTCTCCGCCACAGAGGCCCACTTCGACACCACGAC |
| | | GCCCTTCGCGGCAGTGGTGATCGCCTTGATGGGCACTGTAGCTCAGATGGAGCTAGAGG |
| | | CCATCAAGGAGCGGAACCGGAGCGCCGCTCACTTCAACATCCGGGCGGGCAAGTACCGA |
| | | GGGAGTCTGCCTCCCTGGGGTTACTTGCCCACCCGGGTGGATGGTGAGTGGCGCCTGGT |
| | | TCCGGACCCTGTCCAGAGGGAGCGGATCTTGGAGGTCTACCACCGTGTGGTAGACAACC |
| | | ATGAGCCGCTCCATCTGGTGGCCCACGATCTCAATCGGCGCGGAGTGCTGTCGCCCAAG |
| | | GACTACTTCGCCCAGCTCCAGGGGGGGGAGCCCCAGGGGGGGGAGTGGTCCGCGACAGC |
| | | ACTCAAGCGCTCGATGATATCGGAGGCCATGCTCGGCTACGCAACGCTTAACGGCAAGA |
| | | CTGTTCGCGATGACGATGGGGCTCCGCTCGTGAGAGCGGAGCCCATTCTCACTCGCGAG |
| | | CAGCTTGAAGCGTTGCGAGCCGAGCTGGTGAAGACATCGCGGGCTAAGCCCGCGGTGTC |
| | | TACACCATCTTTGTTGCTTCGGGTCCTCTTCTGCGCTGTATGCGGGGAGCCCGCATACA |
| | | AGTTCGCAGGAGGGGGCCGGAAGCACCCACGGTACCGTTGCCGCAGTATGGGTTTTCCC |
| | | AAGCACTGCGGCAACGGTACCGTGGCCATGGCTGAGTGGGACGCCTTCTGCGAGGAGCA |
| | | AGTGCTGGATCTGCTCGGCGACGCCGAGCGCCTGGAGAAGGTGTGGGTCGCCGGGTCAG |
| | | ATTCAGCAGTTGAACTCGCAGAGGTGAACGCTGAGCTCGTGGACCTCACCTCGCTAATC |
| | | GGGAGTCCAGCGTATCGCGCGGGGTCTCCCCAGCGCGAGGCGCTGGACGCCCGAATAGC |
| | | GGCGCTGGCCGCCCGCCAGGAGGAGCTGGAGGGCCTGGAGGCCAGGCCCTCCGGCTGGG |
| | | AGTGGCGGGAGACCGGCCAGCGCTTCGGAGATTGGTGGCGCGAGCAGGACACCGCAGCC |
| | | AAGAACACTTGGCTGCGGTCCATGAACGTGCGGCTAACCTTCGACGTGCGCGGGGGCTT |
| | | GACCCGGACCATCGACTTCGGTGACCTTCAGGAGTATGAACAGCACCTGAGGTTGGGGT |
| | | CCGTGGTCGAGCGCCTGCACACGGGGATGAGCTGATAACTCGAGCTGGTACTGCATGCA |
| | | CGCAATGCTAGCTGCCCCTTTCCCGTCCTGGGTACCCCGAGTCTCCCCCGACCTCGGGT |
| | | CCCAGGTATGCTCCCACCTCCACCTGCCCCACTCACCACCTCTGCTAGTTCCAGACACC |
| | | TCCCAAGCACGCAGCAATGCAGCTCAAAACGCTTAGCCTAGCCACACCCCCACGGGAAA |
| | | CAGCAGTGATTAACCTTTAGCAATAAACGAAAGTTTAACTAAGCTATACTAACCCCAGG |
| | | GTTGGTCAATTTCGTGCCAGCCACACCCTGGAGCTAGCAAAAAAAAAAAAAAAAAAAAA |
| | | AAAAAAAAAgcatatgactAAAAAAAAAAAAAAAAAAAAAAAAAAAAAAAAAAAAAAAA |
| | | AAAAAAAAAAAAAAAAAAAAAAAAAAAAAAtgaagagcaggcctgcatgcaagcttcta |
| | | gcataaccccttggggcctctaaacgggtcttgaggggttttttgggtgtaatcatggt |
| | | catagctgtttcctgtgtgaacagcctggggctcaccttcgggtgggcctttctgcggg |
| | | tgcctaatgagtgagctaactcacattaattgcgttgcgctcactgcccgctttccagt |
| | | cgggaaacctgtcgtgccagctgcattaatgaatcggccaacgcgcggggagaggcggt |
| | | ttgcgtattgggcgccgaagaaaggcccacccgtgaaggtgagcctcttccgcttcctc |
| | | gctcactgactcgctgcgctcggtcgttcggctgcggcgagcggtatcagctcactcaa |
| | | aggcggtaatacggttatccacagaatcaggggataacgcaggaaagaacatgtgagca |
| | | aaaggccagcaaaaggccaggaaccgtaaaaaggccgcgttgctggcgtttttccatag |
| | | gctccgcccccctgacgagcatcacaaaaatcgacgctcaagtcagaggtggcgaaacc |
| | | cgacaggactataaagataccaggcgtttccccctggaagctccctcgtgcgctctcct |
| | | gttccgaccctgccgcttaccggatacctgtccgcctttctcccttcgggaagcgtggc |
| | | gctttctcatagctcacgctgtaggtatctcagttcggtgtaggtcgttcgctccaagc |
| | | tgggctgtgtgcacgaaccccccgttcagcccgaccgctgcgccttatccggtaactat |
| | | cgtcttgagtccaacccggtaagacacgacttatcgccactggcagcagccactggtaa |
| | | caggattagcagagcgaggtatgtaggcggtgctacagagttcttgaagtggtggccta |
| | | actacggctacactagaagaacagtatttggtatctgcgctctgctgaagccagttacc |
| | | ttcggaaaaagagttggtagctcttgatccggcaaacaaaccaccgctggtagcggtgg |
| | | tttttttgtttgcaagcagcagattacgcgcagaaaaaaaggatctcaagaagatcctt |
| | | tgatcttttctacggggtctgacgctcagtggaacgaaaactcacgttaagggattttg |
| | | gtcatgagattatcaaaaaggatcttcacctagatccttttaaattaaaaatgaagttt |
| | | taaatcaagcccaatctgaataatgttacaaccaattaaccaattctgattagaaaaac |
| | | tcatcgagcatcaaatgaaactgcaatttattcatatcaggattatcaataccatattt |
| | | ttgaaaaagccgtttctgtaatgaaggagaaaactcaccgaggcagttccataggatgg |
| | | caagatcctggtatcggtctgcgattccgactcgtccaacatcaatacaacctattaat |
| | | ttcccctcgtcaaaaataaggttatcaagtgagaaatcaccatgagtgacgactgaatc |
| | | cggtgagaatggcaaaagtttatgcatttctttccagacttgttcaacaggccagccat |
| | | tacgctcgtcatcaaaatcactcgcatcaaccaaaccgttattcattcgtgattgcgcc |
| | | tgagcgagacgaaatacgcgatcgctgttaaaaggacaattacaaacaggaatcgaatg |
| | | caaccggcgcaggaacactgccagcgcatcaacaatattttcacctgaatcaggatatt |
| | | cttctaatacctggaatgctgtttttccggggatcgcagtggtgagtaaccatgcatca |
| | | tcaggagtacggataaaatgcttgatggtcggaagaggcataaattccgtcagccagtt |
| | | tagtctgaccatctcatctgtaacatcattggcaacgctacctttgccatgtttcagaa |
| | | acaactctggcgcatcgggcttcccatacaagcgatagattgtcgcacctgattgcccg |
| | | acattatcgcgagcccatttatacccatataaatcagcatccatgttggaatttaatcg |
| | | cggcctcgacgtttcccgttgaatatggctcataacaccccttgtattactgtttatgt |
| | | aagcagacagttttattgttcatgatgatatatttttatcttgtgcaatgtaacatcag |
| | | agattttgagacacgggccagagctgca |
| 625 | PL1709 | MPKKKRKVQPRFAAAGSGKDKKSDGKDSQKKGSGRALVVIRLSRVTDATTSPERQLESC |
| | _AA | QQLCAQRGWDVVGVAEDLDVSGAVDPFDRKRRPNLARWLAFEEQPFDVIVAYRVDRLTR |
| | | SIRHLQQLVHWAEDHKKLVVSATEAHFDTTTPFAAVVIALMGTVAQMELEAIKERNRSA |
| | | AHFNIRAGKYRGSLPPWGYLPTRVDGEWRLVPDPVQRERILEVYHRVVDNHEPLHLVAH |
| | | DLNRRGVLSPKDYFAQLQGREPQGREWSATALKRSMISEAMLGYATLNGKTVRDDDGAP |
| | | LVRAEPILTREQLEALRAELVKTSRAKPAVSTPSLLLRVLFCAVCGEPAYKFAGGGRKH |
| | | PRYRCRSMGFPKHCGNGTVAMAEWDAFCEEQVLDLLGDAERLEKVWVAGSDSAVELAEV |
| | | NAELVDLTSLIGSPAYRAGSPQREALDARIAALAARQEELEGLEARPSGWEWRETGQRF |
| | | GDWWREQDTAAKNTWLRSMNVRLTFDVRGGLTRTIDFGDLQEYEQHLRLGSWERLHTGM |
| | | S* |
| 626 | PL1709_ | ATGCCCAAGAAGAAGAGAAAGGTGCAACCGCGGTTCGCGGCCGCTGGCAGCGGCAAGGA |
| | DNA | CAAGAAATCCGATGGCAAGGACAGCCAGAAAAAAGGCAGCGGCAGAGCCCTGGTGGTGA |
| | | TCAGACTGAGCAGAGTGACCGACGCCACCACCAGCCCCGAGAGACAGCTGGAGAGCTGC |
| | | CAGCAGCTGTGCGCCCAGAGAGGCTGGGACGTGGTGGGCGTGGCCGAGGACCTGGACGT |
| | | GAGCGGCGCCGTGGACCCCTTCGACAGAAAGAGAAGACCCAACCTGGCCAGATGGCTGG |
| | | CCTTCGAGGAGCAGCCCTTCGACGTGATCGTGGCCTACAGAGTGGACAGACTGACCAGA |
| | | AGCATCAGACACCTGCAGCAGCTGGTGCACTGGGCCGAGGACCACAAGAAGCTGGTGGT |
| | | GAGCGCCACCGAGGCCCACTTCGACACCACCACCCCCTTCGCCGCCGTGGTGATCGCCC |
| | | TGATGGGCACCGTGGCCCAGATGGAGCTGGAGGCCATCAAGGAGAGAAACAGAAGCGCC |
| | | GCCCACTTCAACATCAGAGCCGGCAAGTACAGAGGCAGCCTGCCCCCCTGGGGCTACCT |
| | | GCCCACCAGAGTGGACGGCGAGTGGAGACTGGTGCCCGACCCCGTGCAGAGAGAGAGAA |
| | | TCCTGGAGGTGTACCACAGAGTGGTGGACAACCACGAGCCCCTGCACCTGGTGGCCCAC |
| | | GACCTGAACAGAAGAGGCGTGCTGAGCCCCAAGGACTACTTCGCCCAGCTGCAGGGCAG |
| | | AGAGCCCCAGGGCAGAGAGTGGAGCGCCACCGCCCTGAAGAGAAGCATGATCAGCGAGG |
| | | CCATGCTGGGCTACGCCACCCTGAACGGCAAGACCGTGAGAGACGACGACGGCGCCCCC |
| | | CTGGTGAGAGCCGAGCCCATCCTGACCAGAGAGCAGCTGGAGGCCCTGAGAGCCGAGCT |
| | | GGTGAAGACCAGCAGAGCCAAGCCCGCCGTGAGCACCCCCAGCCTGCTGCTGAGAGTGC |
| | | TGTTCTGCGCCGTGTGCGGCGAGCCCGCCTACAAGTTCGCCGGCGGCGGCAGAAAGCAC |
| | | CCCAGATACAGATGCAGAAGCATGGGCTTCCCCAAGCACTGCGGCAACGGCACCGTGGC |
| | | CATGGCCGAGTGGGACGCCTTCTGCGAGGAGCAGGTGCTGGACCTGCTGGGCGACGCCG |
| | | AGAGACTGGAGAAGGTGTGGGTGGCCGGCAGCGACAGCGCCGTGGAGCTGGCCGAGGTG |
| | | AACGCCGAGCTGGTGGACCTGACCAGCCTGATCGGCAGCCCCGCCTACAGAGCCGGCAG |
| | | CCCCCAGAGAGAGGCCCTGGACGCCAGAATCGCCGCCCTGGCCGCCAGACAGGAGGAGC |
| | | TGGAGGGCCTGGAGGCCAGACCCAGCGGCTGGGAGTGGAGAGAGACCGGCCAGAGATTC |
| | | GGCGACTGGTGGAGAGAGCAGGACACCGCCGCCAAGAACACCTGGCTGAGAAGCATGAA |
| | | CGTGAGACTGACCTTCGACGTGAGAGGCGGCCTGACCAGAACCATCGACTTCGGCGACC |
| | | TGCAGGAGTACGAGCAGCACCTGAGACTGGGCAGCGTGGTGGAGAGACTGCACACCGGC |
| | | ATGAGCTGA |
| 627 | PL1929 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKVGSGATNF |
| | | SLLKQAGDVEENPGPPAAKRVKLDGGSRALVVIRLSRVTDATTSPERQLESCQQLCAQR |
| | | GWDVVGVAEDLDVSGAVDPFDRKRRPNLARWLAFEEQPFDVIVAYRVDRLTRSIRHLQQ |
| | | LVHWAEDHKKLVVSATEAHFDTTTPFAAVVIALMGTVAQMELEAIKERNRSAAHFNIRA |
| | | GKYRGSLPPWGYLPTRVDGEWRLVPDPVQRERILEVYHRVVDNHEPLHLVAHDLNRRGV |
| | | LSPKDYFAQLQGREPQGREWSATALKRSMISEAMLGYATLNGKTVRDDDGAPLVRAEPI |
| | | LTREQLEALRAELVKTSRAKPAVSTPSLLLRVLFCAVCGEPAYKFAGGGRKHPRYRCRS |
| | | MGFPKHCGNGTVAMAEWDAFCEEQVLDLLGDAERLEKVWVAGSDSAVELAEVNAELVDL |
| | | TSLIGSPAYRAGSPQREALDARIAALAARQEELEGLEARPSGWEWRETGQRFGDWWREQ |
| | | DTAAKNTWLRSMNVRLTFDVRGGLTRTIDFGDLQEYEQHLRLGSWVERLHTGMSGGSPK |
| | | KKRKV* |
| 628 | PL1929_ | ATGAAGAGAACCGCCGACGGCAGCGAGTTCGAGAGCCCCAAGAAGAAGAGAAAGGTGGA |
| | DNA | CAAGAAGTACAGCATCGGCCTGGACATCGGCACCAACAGCGTGGGCTGGGCCGTGATCA |
| | | CCGACGAGTACAAGGTGCCCAGCAAGAAGTTCAAGGTGCTGGGCAACACCGACAGACAC |
| | | AGCATCAAGAAGAACCTGATCGGCGCCCTGCTGTTCGACAGCGGCGAGACCGCCGAGGC |
| | | CACCAGACTGAAGAGAACCGCCAGAAGAAGATACACCAGAAGAAAGAACAGAATCTGCT |
| | | ACCTGCAGGAGATCTTCAGCAACGAGATGGCCAAGGTGGACGACAGCTTCTTCCACAGA |
| | | CTGGAGGAGAGCTTCCTGGTGGAGGAGGACAAGAAGCACGAGAGACACCCCATCTTCGG |
| | | CAACATCGTGGACGAGGTGGCCTACCACGAGAAGTACCCCACCATCTACCACCTGAGAA |
| | | AGAAGCTGGTGGACAGCACCGACAAGGCCGACCTGAGACTGATCTACCTGGCCCTGGCC |
| | | CACATGATCAAGTTCAGAGGCCACTTCCTGATCGAGGGCGACCTGAACCCCGACAACAG |
| | | CGACGTGGACAAGCTGTTCATCCAGCTGGTGCAGACCTACAACCAGCTGTTCGAGGAGA |
| | | ACCCCATCAACGCCAGCGGCGTGGACGCCAAGGCCATCCTGAGCGCCAGACTGAGCAAG |
| | | AGCAGAAGACTGGAGAACCTGATCGCCCAGCTGCCCGGCGAGAAGAAGAACGGCCTGTT |
| | | CGGCAACCTGATCGCCCTGAGCCTGGGCCTGACCCCCAACTTCAAGAGCAACTTCGACC |
| | | TGGCCGAGGACGCCAAGCTGCAGCTGAGCAAGGACACCTACGACGACGACCTGGACAAC |
| | | CTGCTGGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCCCCCC |
| | | TGAGCGCCAGCATGATCAAGAGATACGACGAGCACCACCAGGACCTGACCCTGCTGAAG |
| | | GCCCTGGTGAGACAGCAGCTGCCCGAGAAGTACAAGGAGATCTTCTTCGACCAGAGCAA |
| | | GAACGGCTACGCCGGCTACATCGACGGCGGCGCCAGCCAGGAGGAGTTCTACAAGTTCA |
| | | TCAAGCCCATCCTGGAGAAGATGGACGGCACCGAGGAGCTGCTGGTGAAGCTGAACAGA |
| | | GAGGACCTGCTGAGAAAGCAGAGAACCTTCGACAACGGCAGCATCCCCCACCAGATCCA |
| | | CCTGGGCGAGCTGCACGCCATCCTGAGAAGACAGGAGGACTTCTACCCCTTCCTGAAGG |
| | | ACAACAGAGAGAAGATCGAGAAGATCCTGACCTTCAGAATCCCCTACTACGTGGGCCCC |
| | | CTGGCCAGAGGCAACAGCAGATTCGCCTGGATGACCAGAAAGAGCGAGGAGACCATCAC |
| | | CCCCTGGAACTTCGAGGAGGTGGTGGACAAGGGCGCCAGCGCCCAGAGCTTCATCGAGA |
| | | GAATGACCAACTTCGACAAGAACCTGCCCAACGAGAAGGTGCTGCCCAAGCACAGCCTG |
| | | CTGTACGAGTACTTCACCGTGTACAACGAGCTGACCAAGGTGAAGTACGTGACCGAGGG |
| | | CATGAGAAAGCCCGCCTTCCTGAGCGGCGAGCAGAAGAAGGCCATCGTGGACCTGCTGT |
| | | TCAAGACCAACAGAAAGGTGACCGTGAAGCAGCTGAAGGAGGACTACTTCAAGAAGATC |
| | | GAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGA |
| | | ACGAGGACATCCTGGAGGACATCGTGCTGACCCTGACCCTGTTCGAGGACAGAGAGATG |
| | | ATCGAGGAGAGACTGAAGACCTACGCCCACCTGTTCGACGACAAGGTGATGAAGCAGCT |
| | | GAAGAGAAGAAGATACACCGGCTGGGGCAGACTGAGCAGAAAGCTGATCAACGGCATCA |
| | | GAGACAAGCAGAGCGGCAAGACCATCCTGGACTTCCTGAAGAGTGACGGCTTCGCCAAC |
| | | AGAAACTTCATGCAGCTGATCCACGACGACAGCCTGACCTTCAAGGAGGACATCCAGAA |
| | | GGCCCAGGTGAGCGGCCAGGGCGACAGCCTGCACGAGCACATCGCCAACCTGGCCGGCA |
| | | GCCCCGCCATCAAGAAGGGCATCCTGCAGACCGTGAAGGTGGTGGACGAGCTGGTGAAG |
| | | GTGATGGGCAGACACAAGCCCGAGAACATCGTGATCGAGATGGCCAGAGAGAACCAGAC |
| | | CACCCAGAAGGGCCAGAAGAACAGCAGAGAGAGAATGAAGAGAATCGAGGAGGGCATCA |
| | | AGGAGCTGGGCAGCCAGATCCTGAAGGAGCACCCCGTGGAGAACACCCAGCTGCAGAAC |
| | | GAGAAGCTGTACCTGTACTACCTGCAGAACGGCAGAGACATGTACGTGGACCAGGAGCT |
| | | GGACATCAACAGACTGAGCGACTACGACGTGGACGCCATCGTGCCCCAGAGCTTCCTGA |
| | | AGGACGACAGCATCGACAACAAGGTGCTGACCAGAAGCGACAAGAACAGAGGCAAGAGC |
| | | GACAACGTGCCCAGCGAGGAGGTGGTGAAGAAGATGAAGAACTACTGGAGACAGCTGCT |
| | | GAACGCCAAGCTGATCACCCAGAGAAAGTTCGACAACCTGACCAAGGCCGAGAGAGGCG |
| | | GCCTGAGCGAGCTGGACAAGGCCGGCTTCATCAAGAGACAGCTGGTGGAGACCAGACAG |
| | | ATCACCAAGCACGTGGCCCAGATCCTGGACAGCAGAATGAACACCAAGTACGACGAGAA |
| | | CGACAAGCTGATCAGAGAGGTGAAGGTGATCACCCTGAAGAGTAAGCTGGTGAGCGACT |
| | | TCAGAAAGGACTTCCAGTTCTACAAGGTGAGAGAGATCAACAACTACCACCACGCCCAC |
| | | GACGCCTACCTGAACGCCGTGGTGGGCACCGCCCTGATCAAGAAGTACCCCAAGCTGGA |
| | | GAGCGAGTTCGTGTACGGCGACTACAAGGTGTACGACGTGAGAAAGATGATCGCCAAGA |
| | | GCGAGCAGGAGATCGGCAAGGCCACCGCCAAGTACTTCTTCTACAGCAACATCATGAAC |
| | | TTCTTCAAGACCGAGATCACCCTGGCCAACGGCGAGATCAGAAAGAGACCCCTGATCGA |
| | | GACCAACGGCGAGACCGGCGAGATCGTGTGGGACAAGGGCAGAGACTTCGCCACCGTGA |
| | | GAAAGGTGCTGAGCATGCCCCAGGTGAACATCGTGAAGAAGACCGAGGTGCAGACCGGC |
| | | GGCTTCAGCAAGGAGAGCATCCTGCCCAAGAGAAACAGCGACAAGCTGATCGCCAGAAA |
| | | GAAGGACTGGGACCCCAAGAAGTACGGCGGCTTCGACAGCCCCACCGTGGCCTACAGCG |
| | | TGCTGGTGGTGGCCAAGGTGGAGAAGGGCAAGAGCAAGAAGCTGAAGAGTGTGAAGGAG |
| | | CTGCTGGGCATCACCATCATGGAGAGAAGCAGCTTCGAGAAGAACCCCATCGACTTCCT |
| | | GGAGGCCAAGGGCTACAAGGAGGTGAAGAAGGACCTGATCATCAAGCTGCCCAAGTACA |
| | | GCCTGTTCGAGCTGGAGAACGGCAGAAAGAGAATGCTGGCCAGCGCCGGCGAGCTGCAG |
| | | AAGGGCAACGAGCTGGCCCTGCCCAGCAAGTACGTGAACTTCCTGTACCTGGCCAGCCA |
| | | CTACGAGAAGCTGAAGGGCAGCCCCGAGGACAACGAGCAGAAGCAGCTGTTCGTGGAGC |
| | | AGCACAAGCACTACCTGGACGAGATCATCGAGCAGATCAGCGAGTTCAGCAAGAGAGTG |
| | | ATCCTGGCCGACGCCAACCTGGACAAGGTGCTGAGCGCCTACAACAAGCACAGAGACAA |
| | | GCCCATCAGAGAGCAGGCCGAGAACATCATCCACCTGTTCACCCTGACCAACCTGGGCG |
| | | CCCCCGCCGCCTTCAAGTACTTCGACACCACCATCGACAGAAAGAGATACACCAGCACC |
| | | AAGGAGGTGCTGGACGCCACCCTGATCCACCAGAGCATCACCGGCCTGTACGAGACCAG |
| | | AATCGACCTGAGCCAGCTGGGCGGCGACAGCGGCGGCAGCAGCGGCGGCAGCAGCGGCA |
| | | GCGAGACCCCCGGCACCAGCGAGAGCGCCACCCCCGAGAGCAGCGGCGGCAGCAGCGGC |
| | | GGCAGCAGCACCCTGAACATCGAGGACGAGTACAGACTGCACGAGACCAGCAAGGAGCC |
| | | CGACGTGAGCCTGGGCAGCACCTGGCTGAGCGACTTCCCCCAGGCCTGGGCCGAGACCG |
| | | GCGGCATGGGCCTGGCCGTGAGACAGGCCCCCCTGATCATCCCCCTGAAGGCCACCAGC |
| | | ACCCCCGTGAGCATCAAGCAGTACCCCATGAGCCAGGAGGCCAGACTGGGCATCAAGCC |
| | | CCACATCCAGAGACTGCTGGACCAGGGCATCCTGGTGCCCTGCCAGAGCCCCTGGAACA |
| | | CCCCCCTGCTGCCCGTGAAGAAGCCCGGCACCAACGACTACAGACCCGTGCAGGACCTG |
| | | AGAGAGGTGAACAAGAGAGTGGAGGACATCCACCCCACCGTGCCCAACCCCTACAACCT |
| | | GCTGAGCGGCCTGCCCCCCAGCCACCAGTGGTACACCGTGCTGGACCTGAAGGACGCCT |
| | | TCTTCTGCCTGAGACTGCACCCCACCAGCCAGCCCCTGTTCGCCTTCGAGTGGAGAGAC |
| | | CCCGAGATGGGCATCAGCGGCCAGCTGACCTGGACCAGACTGCCCCAGGGCTTCAAGAA |
| | | CAGCCCCACCCTGTTCAACGAGGCCCTGCACAGAGACCTGGCCGACTTCAGAATCCAGC |
| | | ACCCCGACCTGATCCTGCTGCAGTACGTGGACGACCTGCTGCTGGCCGCCACCAGCGAG |
| | | CTGGACTGCCAGCAGGGCACCAGAGCCCTGCTGCAGACCCTGGGCAACCTGGGCTACAG |
| | | AGCCAGCGCCAAGAAGGCCCAGATCTGCCAGAAGCAGGTGAAGTACCTGGGCTACCTGC |
| | | TGAAGGAGGGCCAGAGATGGCTGACCGAGGCCAGAAAGGAGACCGTGATGGGCCAGCCC |
| | | ACCCCCAAGACCCCCAGACAGCTGAGAGAGTTCCTGGGCAAGGCCGGCTTCTGCAGACT |
| | | GTTCATCCCCGGCTTCGCCGAGATGGCCGCCCCCCTGTACCCCCTGACCAAGCCCGGCA |
| | | CCCTGTTCAACTGGGGCCCCGACCAGCAGAAGGCCTACCAGGAGATCAAGCAGGCCCTG |
| | | CTGACCGCCCCCGCCCTGGGCCTGCCCGACCTGACCAAGCCCTTCGAGCTGTTCGTGGA |
| | | CGAGAAGCAGGGCTACGCCAAGGGCGTGCTGACCCAGAAGCTGGGCCCCTGGAGAAGAC |
| | | CCGTGGCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATGGGCCA |
| | | GCCCCTGGTGATCCTGGCCCCCCACGCCGTGGAGGCCCTGGTGAAGCAGCCCCCCGACA |
| | | GATGGCTGAGCAACGCCAGAATGACCCACTACCAGGCCCTGCTGCTGGACACCGACAGA |
| | | GTGCAGTTCGGCCCCGTGGTGGCCCTGAACCCCGCCACCCTGCTGCCCCTGCCCGAGGA |
| | | GGGCCTGCAGCACAACTGCCTGGACATCCTGGCCGAGGCCCACGGCACCAGACCCGACC |
| | | TGACCGACCAGCCCCTGCCCGACGCCGACCACACCTGGTACACCGACGGCAGCAGCCTG |
| | | CTGCAGGAGGGCCAGAGAAAGGCCGGCGCCGCCGTGACCACCGAGACCGAGGTGATCTG |
| | | GGCCAAGGCCCTGCCCGCCGGCACCAGCGCCCAGAGAGCCGAGCTGATCGCCCTGACCC |
| | | AGGCCCTGAAGATGGCCGAGGGCAAGAAGCTGAACGTGTACACCGACAGCAGATACGCC |
| | | TTCGCCACCGCCCACATCCACGGCGAGATCTACAGAAGAAGAGGCTGGCTGACCAGCGA |
| | | GGGCAAGGAGATCAAGAACAAGGACGAGATCCTGGCCCTGCTGAAGGCCCTGTTCCTGC |
| | | CCAAGAGACTGAGCATCATCCACTGCCCCGGCCACCAGAAGGGCCACAGCGCCGAGGCC |
| | | AGAGGCAACAGAATGGCCGACCAGGCCGCCAGAAAGGCCGCCATCACCGAGACCCCCGA |
| | | CACCAGCACCCTGCTGATCGAGAACAGCAGCCCCAGCGGCGGCAGCAAGAGAACCGCCG |
| | | ACGGCAGCGAGTTCGAGCCCAAGAAGAAGAGAAAGGTGGGCAGCGGCGCCACCAACTTC |
| | | AGCCTGCTGAAGCAGGCCGGCGACGTGGAGGAGAACCCCGGCCCCcctgctgctaagag |
| | | agtgaaactggatggcggcagcAGAGCCCTGGTGGTGATCAGACTGAGCAGAGTGACCG |
| | | ACGCCACCACCAGCCCCGAGAGACAGCTGGAGAGCTGCCAGCAGCTGTGCGCCCAGAGA |
| | | GGCTGGGACGTGGTGGGCGTGGCCGAGGACCTGGACGTGAGCGGCGCCGTGGACCCCTT |
| | | CGACAGAAAGAGAAGACCCAACCTGGCCAGATGGCTGGCCTTCGAGGAGCAGCCCTTCG |
| | | ACGTGATCGTGGCCTACAGAGTGGACAGACTGACCAGAAGCATCAGACACCTGCAGCAG |
| | | CTGGTGCACTGGGCCGAGGACCACAAGAAGCTGGTGGTGAGCGCCACCGAGGCCCACTT |
| | | CGACACCACCACCCCCTTCGCCGCCGTGGTGATCGCCCTGATGGGCACCGTGGCCCAGA |
| | | TGGAGCTGGAGGCCATCAAGGAGAGAAACAGAAGCGCCGCCCACTTCAACATCAGAGCC |
| | | GGCAAGTACAGAGGCAGCCTGCCCCCCTGGGGCTACCTGCCCACCAGAGTGGACGGCGA |
| | | GTGGAGACTGGTGCCCGACCCCGTGCAGAGAGAGAGAATCCTGGAGGTGTACCACAGAG |
| | | TGGTGGACAACCACGAGCCCCTGCACCTGGTGGCCCACGACCTGAACAGAAGAGGCGTG |
| | | CTGAGCCCCAAGGACTACTTCGCCCAGCTGCAGGGCAGAGAGCCCCAGGGCAGAGAGTG |
| | | GAGCGCCACCGCCCTGAAGAGAAGCATGATCAGCGAGGCCATGCTGGGCTACGCCACCC |
| | | TGAACGGCAAGACCGTGAGAGACGACGACGGCGCCCCCCTGGTGAGAGCCGAGCCCATC |
| | | CTGACCAGAGAGCAGCTGGAGGCCCTGAGAGCCGAGCTGGTGAAGACCAGCAGAGCCAA |
| | | GCCCGCCGTGAGCACCCCCAGCCTGCTGCTGAGAGTGCTGTTCTGCGCCGTGTGCGGCG |
| | | AGCCCGCCTACAAGTTCGCCGGCGGCGGCAGAAAGCACCCCAGATACAGATGCAGAAGC |
| | | ATGGGCTTCCCCAAGCACTGCGGCAACGGCACCGTGGCCATGGCCGAGTGGGACGCCTT |
| | | CTGCGAGGAGCAGGTGCTGGACCTGCTGGGCGACGCCGAGAGACTGGAGAAGGTGTGGG |
| | | TGGCCGGCAGCGACAGCGCCGTGGAGCTGGCCGAGGTGAACGCCGAGCTGGTGGACCTG |
| | | ACCAGCCTGATCGGCAGCCCCGCCTACAGAGCCGGCAGCCCCCAGAGAGAGGCCCTGGA |
| | | CGCCAGAATCGCCGCCCTGGCCGCCAGACAGGAGGAGCTGGAGGGCCTGGAGGCCAGAC |
| | | CCAGCGGCTGGGAGTGGAGAGAGACCGGCCAGAGATTCGGCGACTGGTGGAGAGAGCAG |
| | | GACACCGCCGCCAAGAACACCTGGCTGAGAAGCATGAACGTGAGACTGACCTTCGACGT |
| | | GAGAGGCGGCCTGACCAGAACCATCGACTTCGGCGACCTGCAGGAGTACGAGCAGCACC |
| | | TGAGACTGGGCAGCGTGGTGGAGAGACTGCACACCGGCATGAGCGGCGGCAGCCCCAAG |
| | | AAGAAGAGAAAGGTGTGA |
| 629 | PL1930 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLWVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKVGSGATNF |
| | | SLLKQAGDVEENPGPPAAKRVKLDGGSKDKKSDGKDSQKKGSRALWIRLSRVTDATTSP |
| | | ERQLESCQQLCAQRGWDVVGVAEDLDVSGAVDPFDRKRRPNLARWLAFEEQPFDVIVAY |
| | | RVDRLTRSIRHLQQLVHWAEDHKKLVVSATEAHFDTTTPFAAVVIALMGTVAQMELEAI |
| | | KERNRSAAHFNIRAGKYRGSLPPWGYLPTRVDGEWRLVPDPVQRERILEVYHRVVDNHE |
| | | PLHLVAHDLNRRGVLSPKDYFAQLQGREPQGREWSATALKRSMISEAMLGYATLNGKTV |
| | | RDDDGAPLVRAEPILTREQLEALRAELVKTSRAKPAVSTPSLLLRVLFCAVCGEPAYKF |
| | | AGGGRKHPRYRCRSMGFPKHCGNGTVAMAEWDAFCEEQVLDLLGDAERLEKVWVAGSDS |
| | | AVELAEVNAELVDLTSLIGSPAYRAGSPQREALDARIAALAARQEELEGLEARPSGWEW |
| | | RETGQRFGDWWREQDTAAKNTWLRSMNVRLTFDVRGGLTRTIDFGDLQEYEQHLRLGSV |
| | | VERLHTGMSGGSPKKKRKV* |
| 630 | PL1930_ | ATGAAGAGAACCGCCGACGGCAGCGAGTTCGAGAGCCCCAAGAAGAAGAGAAAGGTGGA |
| | DNA | CAAGAAGTACAGCATCGGCCTGGACATCGGCACCAACAGCGTGGGCTGGGCCGTGATCA |
| | | CCGACGAGTACAAGGTGCCCAGCAAGAAGTTCAAGGTGCTGGGCAACACCGACAGACAC |
| | | AGCATCAAGAAGAACCTGATCGGCGCCCTGCTGTTCGACAGCGGCGAGACCGCCGAGGC |
| | | CACCAGACTGAAGAGAACCGCCAGAAGAAGATACACCAGAAGAAAGAACAGAATCTGCT |
| | | ACCTGCAGGAGATCTTCAGCAACGAGATGGCCAAGGTGGACGACAGCTTCTTCCACAGA |
| | | CTGGAGGAGAGCTTCCTGGTGGAGGAGGACAAGAAGCACGAGAGACACCCCATCTTCGG |
| | | CAACATCGTGGACGAGGTGGCCTACCACGAGAAGTACCCCACCATCTACCACCTGAGAA |
| | | AGAAGCTGGTGGACAGCACCGACAAGGCCGACCTGAGACTGATCTACCTGGCCCTGGCC |
| | | CACATGATCAAGTTCAGAGGCCACTTCCTGATCGAGGGCGACCTGAACCCCGACAACAG |
| | | CGACGTGGACAAGCTGTTCATCCAGCTGGTGCAGACCTACAACCAGCTGTTCGAGGAGA |
| | | ACCCCATCAACGCCAGCGGCGTGGACGCCAAGGCCATCCTGAGCGCCAGACTGAGCAAG |
| | | AGCAGAAGACTGGAGAACCTGATCGCCCAGCTGCCCGGCGAGAAGAAGAACGGCCTGTT |
| | | CGGCAACCTGATCGCCCTGAGCCTGGGCCTGACCCCCAACTTCAAGAGCAACTTCGACC |
| | | TGGCCGAGGACGCCAAGCTGCAGCTGAGCAAGGACACCTACGACGACGACCTGGACAAC |
| | | CTGCTGGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCCCCCC |
| | | TGAGCGCCAGCATGATCAAGAGATACGACGAGCACCACCAGGACCTGACCCTGCTGAAG |
| | | GCCCTGGTGAGACAGCAGCTGCCCGAGAAGTACAAGGAGATCTTCTTCGACCAGAGCAA |
| | | GAACGGCTACGCCGGCTACATCGACGGCGGCGCCAGCCAGGAGGAGTTCTACAAGTTCA |
| | | TCAAGCCCATCCTGGAGAAGATGGACGGCACCGAGGAGCTGCTGGTGAAGCTGAACAGA |
| | | GAGGACCTGCTGAGAAAGCAGAGAACCTTCGACAACGGCAGCATCCCCCACCAGATCCA |
| | | CCTGGGCGAGCTGCACGCCATCCTGAGAAGACAGGAGGACTTCTACCCCTTCCTGAAGG |
| | | ACAACAGAGAGAAGATCGAGAAGATCCTGACCTTCAGAATCCCCTACTACGTGGGCCCC |
| | | CTGGCCAGAGGCAACAGCAGATTCGCCTGGATGACCAGAAAGAGCGAGGAGACCATCAC |
| | | CCCCTGGAACTTCGAGGAGGTGGTGGACAAGGGCGCCAGCGCCCAGAGCTTCATCGAGA |
| | | GAATGACCAACTTCGACAAGAACCTGCCCAACGAGAAGGTGCTGCCCAAGCACAGCCTG |
| | | CTGTACGAGTACTTCACCGTGTACAACGAGCTGACCAAGGTGAAGTACGTGACCGAGGG |
| | | CATGAGAAAGCCCGCCTTCCTGAGCGGCGAGCAGAAGAAGGCCATCGTGGACCTGCTGT |
| | | TCAAGACCAACAGAAAGGTGACCGTGAAGCAGCTGAAGGAGGACTACTTCAAGAAGATC |
| | | GAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGA |
| | | ACGAGGACATCCTGGAGGACATCGTGCTGACCCTGACCCTGTTCGAGGACAGAGAGATG |
| | | ATCGAGGAGAGACTGAAGACCTACGCCCACCTGTTCGACGACAAGGTGATGAAGCAGCT |
| | | GAAGAGAAGAAGATACACCGGCTGGGGCAGACTGAGCAGAAAGCTGATCAACGGCATCA |
| | | GAGACAAGCAGAGCGGCAAGACCATCCTGGACTTCCTGAAGAGTGACGGCTTCGCCAAC |
| | | AGAAACTTCATGCAGCTGATCCACGACGACAGCCTGACCTTCAAGGAGGACATCCAGAA |
| | | GGCCCAGGTGAGCGGCCAGGGCGACAGCCTGCACGAGCACATCGCCAACCTGGCCGGCA |
| | | GCCCCGCCATCAAGAAGGGCATCCTGCAGACCGTGAAGGTGGTGGACGAGCTGGTGAAG |
| | | GTGATGGGCAGACACAAGCCCGAGAACATCGTGATCGAGATGGCCAGAGAGAACCAGAC |
| | | CACCCAGAAGGGCCAGAAGAACAGCAGAGAGAGAATGAAGAGAATCGAGGAGGGCATCA |
| | | AGGAGCTGGGCAGCCAGATCCTGAAGGAGCACCCCGTGGAGAACACCCAGCTGCAGAAC |
| | | GAGAAGCTGTACCTGTACTACCTGCAGAACGGCAGAGACATGTACGTGGACCAGGAGCT |
| | | GGACATCAACAGACTGAGCGACTACGACGTGGACGCCATCGTGCCCCAGAGCTTCCTGA |
| | | AGGACGACAGCATCGACAACAAGGTGCTGACCAGAAGCGACAAGAACAGAGGCAAGAGC |
| | | GACAACGTGCCCAGCGAGGAGGTGGTGAAGAAGATGAAGAACTACTGGAGACAGCTGCT |
| | | GAACGCCAAGCTGATCACCCAGAGAAAGTTCGACAACCTGACCAAGGCCGAGAGAGGCG |
| | | GCCTGAGCGAGCTGGACAAGGCCGGCTTCATCAAGAGACAGCTGGTGGAGACCAGACAG |
| | | ATCACCAAGCACGTGGCCCAGATCCTGGACAGCAGAATGAACACCAAGTACGACGAGAA |
| | | CGACAAGCTGATCAGAGAGGTGAAGGTGATCACCCTGAAGAGTAAGCTGGTGAGCGACT |
| | | TCAGAAAGGACTTCCAGTTCTACAAGGTGAGAGAGATCAACAACTACCACCACGCCCAC |
| | | GACGCCTACCTGAACGCCGTGGTGGGCACCGCCCTGATCAAGAAGTACCCCAAGCTGGA |
| | | GAGCGAGTTCGTGTACGGCGACTACAAGGTGTACGACGTGAGAAAGATGATCGCCAAGA |
| | | GCGAGCAGGAGATCGGCAAGGCCACCGCCAAGTACTTCTTCTACAGCAACATCATGAAC |
| | | TTCTTCAAGACCGAGATCACCCTGGCCAACGGCGAGATCAGAAAGAGACCCCTGATCGA |
| | | GACCAACGGCGAGACCGGCGAGATCGTGTGGGACAAGGGCAGAGACTTCGCCACCGTGA |
| | | GAAAGGTGCTGAGCATGCCCCAGGTGAACATCGTGAAGAAGACCGAGGTGCAGACCGGC |
| | | GGCTTCAGCAAGGAGAGCATCCTGCCCAAGAGAAACAGCGACAAGCTGATCGCCAGAAA |
| | | GAAGGACTGGGACCCCAAGAAGTACGGCGGCTTCGACAGCCCCACCGTGGCCTACAGCG |
| | | TGCTGGTGGTGGCCAAGGTGGAGAAGGGCAAGAGCAAGAAGCTGAAGAGTGTGAAGGAG |
| | | CTGCTGGGCATCACCATCATGGAGAGAAGCAGCTTCGAGAAGAACCCCATCGACTTCCT |
| | | GGAGGCCAAGGGCTACAAGGAGGTGAAGAAGGACCTGATCATCAAGCTGCCCAAGTACA |
| | | GCCTGTTCGAGCTGGAGAACGGCAGAAAGAGAATGCTGGCCAGCGCCGGCGAGCTGCAG |
| | | AAGGGCAACGAGCTGGCCCTGCCCAGCAAGTACGTGAACTTCCTGTACCTGGCCAGCCA |
| | | CTACGAGAAGCTGAAGGGCAGCCCCGAGGACAACGAGCAGAAGCAGCTGTTCGTGGAGC |
| | | AGCACAAGCACTACCTGGACGAGATCATCGAGCAGATCAGCGAGTTCAGCAAGAGAGTG |
| | | ATCCTGGCCGACGCCAACCTGGACAAGGTGCTGAGCGCCTACAACAAGCACAGAGACAA |
| | | GCCCATCAGAGAGCAGGCCGAGAACATCATCCACCTGTTCACCCTGACCAACCTGGGCG |
| | | CCCCCGCCGCCTTCAAGTACTTCGACACCACCATCGACAGAAAGAGATACACCAGCACC |
| | | AAGGAGGTGCTGGACGCCACCCTGATCCACCAGAGCATCACCGGCCTGTACGAGACCAG |
| | | AATCGACCTGAGCCAGCTGGGCGGCGACAGCGGCGGCAGCAGCGGCGGCAGCAGCGGCA |
| | | GCGAGACCCCCGGCACCAGCGAGAGCGCCACCCCCGAGAGCAGCGGCGGCAGCAGCGGC |
| | | GGCAGCAGCACCCTGAACATCGAGGACGAGTACAGACTGCACGAGACCAGCAAGGAGCC |
| | | CGACGTGAGCCTGGGCAGCACCTGGCTGAGCGACTTCCCCCAGGCCTGGGCCGAGACCG |
| | | GCGGCATGGGCCTGGCCGTGAGACAGGCCCCCCTGATCATCCCCCTGAAGGCCACCAGC |
| | | ACCCCCGTGAGCATCAAGCAGTACCCCATGAGCCAGGAGGCCAGACTGGGCATCAAGCC |
| | | CCACATCCAGAGACTGCTGGACCAGGGCATCCTGGTGCCCTGCCAGAGCCCCTGGAACA |
| | | CCCCCCTGCTGCCCGTGAAGAAGCCCGGCACCAACGACTACAGACCCGTGCAGGACCTG |
| | | AGAGAGGTGAACAAGAGAGTGGAGGACATCCACCCCACCGTGCCCAACCCCTACAACCT |
| | | GCTGAGCGGCCTGCCCCCCAGCCACCAGTGGTACACCGTGCTGGACCTGAAGGACGCCT |
| | | TCTTCTGCCTGAGACTGCACCCCACCAGCCAGCCCCTGTTCGCCTTCGAGTGGAGAGAC |
| | | CCCGAGATGGGCATCAGCGGCCAGCTGACCTGGACCAGACTGCCCCAGGGCTTCAAGAA |
| | | CAGCCCCACCCTGTTCAACGAGGCCCTGCACAGAGACCTGGCCGACTTCAGAATCCAGC |
| | | ACCCCGACCTGATCCTGCTGCAGTACGTGGACGACCTGCTGCTGGCCGCCACCAGCGAG |
| | | CTGGACTGCCAGCAGGGCACCAGAGCCCTGCTGCAGACCCTGGGCAACCTGGGCTACAG |
| | | AGCCAGCGCCAAGAAGGCCCAGATCTGCCAGAAGCAGGTGAAGTACCTGGGCTACCTGC |
| | | TGAAGGAGGGCCAGAGATGGCTGACCGAGGCCAGAAAGGAGACCGTGATGGGCCAGCCC |
| | | ACCCCCAAGACCCCCAGACAGCTGAGAGAGTTCCTGGGCAAGGCCGGCTTCTGCAGACT |
| | | GTTCATCCCCGGCTTCGCCGAGATGGCCGCCCCCCTGTACCCCCTGACCAAGCCCGGCA |
| | | CCCTGTTCAACTGGGGCCCCGACCAGCAGAAGGCCTACCAGGAGATCAAGCAGGCCCTG |
| | | CTGACCGCCCCCGCCCTGGGCCTGCCCGACCTGACCAAGCCCTTCGAGCTGTTCGTGGA |
| | | CGAGAAGCAGGGCTACGCCAAGGGCGTGCTGACCCAGAAGCTGGGCCCCTGGAGAAGAC |
| | | CCGTGGCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATGGGCCA |
| | | GCCCCTGGTGATCCTGGCCCCCCACGCCGTGGAGGCCCTGGTGAAGCAGCCCCCCGACA |
| | | GATGGCTGAGCAACGCCAGAATGACCCACTACCAGGCCCTGCTGCTGGACACCGACAGA |
| | | GTGCAGTTCGGCCCCGTGGTGGCCCTGAACCCCGCCACCCTGCTGCCCCTGCCCGAGGA |
| | | GGGCCTGCAGCACAACTGCCTGGACATCCTGGCCGAGGCCCACGGCACCAGACCCGACC |
| | | TGACCGACCAGCCCCTGCCCGACGCCGACCACACCTGGTACACCGACGGCAGCAGCCTG |
| | | CTGCAGGAGGGCCAGAGAAAGGCCGGCGCCGCCGTGACCACCGAGACCGAGGTGATCTG |
| | | GGCCAAGGCCCTGCCCGCCGGCACCAGCGCCCAGAGAGCCGAGCTGATCGCCCTGACCC |
| | | AGGCCCTGAAGATGGCCGAGGGCAAGAAGCTGAACGTGTACACCGACAGCAGATACGCC |
| | | TTCGCCACCGCCCACATCCACGGCGAGATCTACAGAAGAAGAGGCTGGCTGACCAGCGA |
| | | GGGCAAGGAGATCAAGAACAAGGACGAGATCCTGGCCCTGCTGAAGGCCCTGTTCCTGC |
| | | CCAAGAGACTGAGCATCATCCACTGCCCCGGCCACCAGAAGGGCCACAGCGCCGAGGCC |
| | | AGAGGCAACAGAATGGCCGACCAGGCCGCCAGAAAGGCCGCCATCACCGAGACCCCCGA |
| | | CACCAGCACCCTGCTGATCGAGAACAGCAGCCCCAGCGGCGGCAGCAAGAGAACCGCCG |
| | | ACGGCAGCGAGTTCGAGCCCAAGAAGAAGAGAAAGGTGGGCAGCGGCGCCACCAACTTC |
| | | AGCCTGCTGAAGCAGGCCGGCGACGTGGAGGAGAACCCCGGCCCCcctgctgctaagag |
| | | agtgaaactggatggcggcagcAAGGACAAGAAATCCGATGGCAAGGACAGCCAGAAAA |
| | | AAGGCAGCAGAGCCCTGGTGGTGATCAGACTGAGCAGAGTGACCGACGCCACCACCAGC |
| | | CCCGAGAGACAGCTGGAGAGCTGCCAGCAGCTGTGCGCCCAGAGAGGCTGGGACGTGGT |
| | | GGGCGTGGCCGAGGACCTGGACGTGAGCGGCGCCGTGGACCCCTTCGACAGAAAGAGAA |
| | | GACCCAACCTGGCCAGATGGCTGGCCTTCGAGGAGCAGCCCTTCGACGTGATCGTGGCC |
| | | TACAGAGTGGACAGACTGACCAGAAGCATCAGACACCTGCAGCAGCTGGTGCACTGGGC |
| | | CGAGGACCACAAGAAGCTGGTGGTGAGCGCCACCGAGGCCCACTTCGACACCACCACCC |
| | | CCTTCGCCGCCGTGGTGATCGCCCTGATGGGCACCGTGGCCCAGATGGAGCTGGAGGCC |
| | | ATCAAGGAGAGAAACAGAAGCGCCGCCCACTTCAACATCAGAGCCGGCAAGTACAGAGG |
| | | CAGCCTGCCCCCCTGGGGCTACCTGCCCACCAGAGTGGACGGCGAGTGGAGACTGGTGC |
| | | CCGACCCCGTGCAGAGAGAGAGAATCCTGGAGGTGTACCACAGAGTGGTGGACAACCAC |
| | | GAGCCCCTGCACCTGGTGGCCCACGACCTGAACAGAAGAGGCGTGCTGAGCCCCAAGGA |
| | | CTACTTCGCCCAGCTGCAGGGCAGAGAGCCCCAGGGCAGAGAGTGGAGCGCCACCGCCC |
| | | TGAAGAGAAGCATGATCAGCGAGGCCATGCTGGGCTACGCCACCCTGAACGGCAAGACC |
| | | GTGAGAGACGACGACGGCGCCCCCCTGGTGAGAGCCGAGCCCATCCTGACCAGAGAGCA |
| | | GCTGGAGGCCCTGAGAGCCGAGCTGGTGAAGACCAGCAGAGCCAAGCCCGCCGTGAGCA |
| | | CCCCCAGCCTGCTGCTGAGAGTGCTGTTCTGCGCCGTGTGCGGCGAGCCCGCCTACAAG |
| | | TTCGCCGGCGGCGGCAGAAAGCACCCCAGATACAGATGCAGAAGCATGGGCTTCCCCAA |
| | | GCACTGCGGCAACGGCACCGTGGCCATGGCCGAGTGGGACGCCTTCTGCGAGGAGCAGG |
| | | TGCTGGACCTGCTGGGCGACGCCGAGAGACTGGAGAAGGTGTGGGTGGCCGGCAGCGAC |
| | | AGCGCCGTGGAGCTGGCCGAGGTGAACGCCGAGCTGGTGGACCTGACCAGCCTGATCGG |
| | | CAGCCCCGCCTACAGAGCCGGCAGCCCCCAGAGAGAGGCCCTGGACGCCAGAATCGCCG |
| | | CCCTGGCCGCCAGACAGGAGGAGCTGGAGGGCCTGGAGGCCAGACCCAGCGGCTGGGAG |
| | | TGGAGAGAGACCGGCCAGAGATTCGGCGACTGGTGGAGAGAGCAGGACACCGCCGCCAA |
| | | GAACACCTGGCTGAGAAGCATGAACGTGAGACTGACCTTCGACGTGAGAGGCGGCCTGA |
| | | CCAGAACCATCGACTTCGGCGACCTGCAGGAGTACGAGCAGCACCTGAGACTGGGCAGC |
| | | GTGGTGGAGAGACTGCACACCGGCATGAGCGGCGGCAGCCCCAAGAAGAAGAGAAAGGT |
| | | GTGA |
| 631 | PL1931 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKVGGSKDKK |
| | | SDGKDSQKKGSGATNFSLLKQAGDVEENPGPPAAKRVKLDGGSKDKKSDGKDSQKKGSR |
| | | ALVVIRLSRVTDATTSPERQLESCQQLCAQRGWDVVGVAEDLDVSGAVDPFDRKRRPNL |
| | | ARWLAFEEQPFDVIVAYRVDRLTRSIRHLQQLVHWAEDHKKLVVSATEAHFDTTTPFAA |
| | | VVIALMGTVAQMELEAIKERNRSAAHFNIRAGKYRGSLPPWGYLPTRVDGEWRLVPDPV |
| | | QRERILEVYHRVVDNHEPLHLVAHDLNRRGVLSPKDYFAQLQGREPQGREWSATALKRS |
| | | MISEAMLGYATLNGKTVRDDDGAPLVRAEPILTREQLEALRAELVKTSRAKPAVSTPSL |
| | | LLRVLFCAVCGEPAYKFAGGGRKHPRYRCRSMGFPKHCGNGTVAMAEWDAFCEEQVLDL |
| | | LGDAERLEKVWVAGSDSAVELAEVNAELVDLTSLIGSPAYRAGSPQREALDARIAALAA |
| | | RQEELEGLEARPSGWEWRETGQRFGDWWREQDTAAKNTWLRSMNVRLTFDVRGGLTRTI |
| | | DFGDLQEYEQHLRLGSVVERLHTGMSGGSPKKKRKV* |
| 632 | PL1931_ | ATGAAGAGAACCGCCGACGGCAGCGAGTTCGAGAGCCCCAAGAAGAAGAGAAAGGTGGA |
| | DNA | CAAGAAGTACAGCATCGGCCTGGACATCGGCACCAACAGCGTGGGCTGGGCCGTGATCA |
| | | CCGACGAGTACAAGGTGCCCAGCAAGAAGTTCAAGGTGCTGGGCAACACCGACAGACAC |
| | | AGCATCAAGAAGAACCTGATCGGCGCCCTGCTGTTCGACAGCGGCGAGACCGCCGAGGC |
| | | CACCAGACTGAAGAGAACCGCCAGAAGAAGATACACCAGAAGAAAGAACAGAATCTGCT |
| | | ACCTGCAGGAGATCTTCAGCAACGAGATGGCCAAGGTGGACGACAGCTTCTTCCACAGA |
| | | CTGGAGGAGAGCTTCCTGGTGGAGGAGGACAAGAAGCACGAGAGACACCCCATCTTCGG |
| | | CAACATCGTGGACGAGGTGGCCTACCACGAGAAGTACCCCACCATCTACCACCTGAGAA |
| | | AGAAGCTGGTGGACAGCACCGACAAGGCCGACCTGAGACTGATCTACCTGGCCCTGGCC |
| | | CACATGATCAAGTTCAGAGGCCACTTCCTGATCGAGGGCGACCTGAACCCCGACAACAG |
| | | CGACGTGGACAAGCTGTTCATCCAGCTGGTGCAGACCTACAACCAGCTGTTCGAGGAGA |
| | | ACCCCATCAACGCCAGCGGCGTGGACGCCAAGGCCATCCTGAGCGCCAGACTGAGCAAG |
| | | AGCAGAAGACTGGAGAACCTGATCGCCCAGCTGCCCGGCGAGAAGAAGAACGGCCTGTT |
| | | CGGCAACCTGATCGCCCTGAGCCTGGGCCTGACCCCCAACTTCAAGAGCAACTTCGACC |
| | | TGGCCGAGGACGCCAAGCTGCAGCTGAGCAAGGACACCTACGACGACGACCTGGACAAC |
| | | CTGCTGGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCCCCCC |
| | | TGAGCGCCAGCATGATCAAGAGATACGACGAGCACCACCAGGACCTGACCCTGCTGAAG |
| | | GCCCTGGTGAGACAGCAGCTGCCCGAGAAGTACAAGGAGATCTTCTTCGACCAGAGCAA |
| | | GAACGGCTACGCCGGCTACATCGACGGCGGCGCCAGCCAGGAGGAGTTCTACAAGTTCA |
| | | TCAAGCCCATCCTGGAGAAGATGGACGGCACCGAGGAGCTGCTGGTGAAGCTGAACAGA |
| | | GAGGACCTGCTGAGAAAGCAGAGAACCTTCGACAACGGCAGCATCCCCCACCAGATCCA |
| | | CCTGGGCGAGCTGCACGCCATCCTGAGAAGACAGGAGGACTTCTACCCCTTCCTGAAGG |
| | | ACAACAGAGAGAAGATCGAGAAGATCCTGACCTTCAGAATCCCCTACTACGTGGGCCCC |
| | | CTGGCCAGAGGCAACAGCAGATTCGCCTGGATGACCAGAAAGAGCGAGGAGACCATCAC |
| | | CCCCTGGAACTTCGAGGAGGTGGTGGACAAGGGCGCCAGCGCCCAGAGCTTCATCGAGA |
| | | GAATGACCAACTTCGACAAGAACCTGCCCAACGAGAAGGTGCTGCCCAAGCACAGCCTG |
| | | CTGTACGAGTACTTCACCGTGTACAACGAGCTGACCAAGGTGAAGTACGTGACCGAGGG |
| | | CATGAGAAAGCCCGCCTTCCTGAGCGGCGAGCAGAAGAAGGCCATCGTGGACCTGCTGT |
| | | TCAAGACCAACAGAAAGGTGACCGTGAAGCAGCTGAAGGAGGACTACTTCAAGAAGATC |
| | | GAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGA |
| | | ACGAGGACATCCTGGAGGACATCGTGCTGACCCTGACCCTGTTCGAGGACAGAGAGATG |
| | | ATCGAGGAGAGACTGAAGACCTACGCCCACCTGTTCGACGACAAGGTGATGAAGCAGCT |
| | | GAAGAGAAGAAGATACACCGGCTGGGGCAGACTGAGCAGAAAGCTGATCAACGGCATCA |
| | | GAGACAAGCAGAGCGGCAAGACCATCCTGGACTTCCTGAAGAGTGACGGCTTCGCCAAC |
| | | AGAAACTTCATGCAGCTGATCCACGACGACAGCCTGACCTTCAAGGAGGACATCCAGAA |
| | | GGCCCAGGTGAGCGGCCAGGGCGACAGCCTGCACGAGCACATCGCCAACCTGGCCGGCA |
| | | GCCCCGCCATCAAGAAGGGCATCCTGCAGACCGTGAAGGTGGTGGACGAGCTGGTGAAG |
| | | GTGATGGGCAGACACAAGCCCGAGAACATCGTGATCGAGATGGCCAGAGAGAACCAGAC |
| | | CACCCAGAAGGGCCAGAAGAACAGCAGAGAGAGAATGAAGAGAATCGAGGAGGGCATCA |
| | | AGGAGCTGGGCAGCCAGATCCTGAAGGAGCACCCCGTGGAGAACACCCAGCTGCAGAAC |
| | | GAGAAGCTGTACCTGTACTACCTGCAGAACGGCAGAGACATGTACGTGGACCAGGAGCT |
| | | GGACATCAACAGACTGAGCGACTACGACGTGGACGCCATCGTGCCCCAGAGCTTCCTGA |
| | | AGGACGACAGCATCGACAACAAGGTGCTGACCAGAAGCGACAAGAACAGAGGCAAGAGC |
| | | GACAACGTGCCCAGCGAGGAGGTGGTGAAGAAGATGAAGAACTACTGGAGACAGCTGCT |
| | | GAACGCCAAGCTGATCACCCAGAGAAAGTTCGACAACCTGACCAAGGCCGAGAGAGGCG |
| | | GCCTGAGCGAGCTGGACAAGGCCGGCTTCATCAAGAGACAGCTGGTGGAGACCAGACAG |
| | | ATCACCAAGCACGTGGCCCAGATCCTGGACAGCAGAATGAACACCAAGTACGACGAGAA |
| | | CGACAAGCTGATCAGAGAGGTGAAGGTGATCACCCTGAAGAGTAAGCTGGTGAGCGACT |
| | | TCAGAAAGGACTTCCAGTTCTACAAGGTGAGAGAGATCAACAACTACCACCACGCCCAC |
| | | GACGCCTACCTGAACGCCGTGGTGGGCACCGCCCTGATCAAGAAGTACCCCAAGCTGGA |
| | | GAGCGAGTTCGTGTACGGCGACTACAAGGTGTACGACGTGAGAAAGATGATCGCCAAGA |
| | | GCGAGCAGGAGATCGGCAAGGCCACCGCCAAGTACTTCTTCTACAGCAACATCATGAAC |
| | | TTCTTCAAGACCGAGATCACCCTGGCCAACGGCGAGATCAGAAAGAGACCCCTGATCGA |
| | | GACCAACGGCGAGACCGGCGAGATCGTGTGGGACAAGGGCAGAGACTTCGCCACCGTGA |
| | | GAAAGGTGCTGAGCATGCCCCAGGTGAACATCGTGAAGAAGACCGAGGTGCAGACCGGC |
| | | GGCTTCAGCAAGGAGAGCATCCTGCCCAAGAGAAACAGCGACAAGCTGATCGCCAGAAA |
| | | GAAGGACTGGGACCCCAAGAAGTACGGCGGCTTCGACAGCCCCACCGTGGCCTACAGCG |
| | | TGCTGGTGGTGGCCAAGGTGGAGAAGGGCAAGAGCAAGAAGCTGAAGAGTGTGAAGGAG |
| | | CTGCTGGGCATCACCATCATGGAGAGAAGCAGCTTCGAGAAGAACCCCATCGACTTCCT |
| | | GGAGGCCAAGGGCTACAAGGAGGTGAAGAAGGACCTGATCATCAAGCTGCCCAAGTACA |
| | | GCCTGTTCGAGCTGGAGAACGGCAGAAAGAGAATGCTGGCCAGCGCCGGCGAGCTGCAG |
| | | AAGGGCAACGAGCTGGCCCTGCCCAGCAAGTACGTGAACTTCCTGTACCTGGCCAGCCA |
| | | CTACGAGAAGCTGAAGGGCAGCCCCGAGGACAACGAGCAGAAGCAGCTGTTCGTGGAGC |
| | | AGCACAAGCACTACCTGGACGAGATCATCGAGCAGATCAGCGAGTTCAGCAAGAGAGTG |
| | | ATCCTGGCCGACGCCAACCTGGACAAGGTGCTGAGCGCCTACAACAAGCACAGAGACAA |
| | | GCCCATCAGAGAGCAGGCCGAGAACATCATCCACCTGTTCACCCTGACCAACCTGGGCG |
| | | CCCCCGCCGCCTTCAAGTACTTCGACACCACCATCGACAGAAAGAGATACACCAGCACC |
| | | AAGGAGGTGCTGGACGCCACCCTGATCCACCAGAGCATCACCGGCCTGTACGAGACCAG |
| | | AATCGACCTGAGCCAGCTGGGGGGCGACAGCGGCGGCAGCAGCGGCGGCAGCAGCGGCA |
| | | GCGAGACCCCCGGCACCAGCGAGAGCGCCACCCCCGAGAGCAGCGGCGGCAGCAGCGGC |
| | | GGCAGCAGCACCCTGAACATCGAGGACGAGTACAGACTGCACGAGACCAGCAAGGAGCC |
| | | CGACGTGAGCCTGGGCAGCACCTGGCTGAGCGACTTCCCCCAGGCCTGGGCCGAGACCG |
| | | GCGGCATGGGCCTGGCCGTGAGACAGGCCCCCCTGATCATCCCCCTGAAGGCCACCAGC |
| | | ACCCCCGTGAGCATCAAGCAGTACCCCATGAGCCAGGAGGCCAGACTGGGCATCAAGCC |
| | | CCACATCCAGAGACTGCTGGACCAGGGCATCCTGGTGCCCTGCCAGAGCCCCTGGAACA |
| | | CCCCCCTGCTGCCCGTGAAGAAGCCCGGCACCAACGACTACAGACCCGTGCAGGACCTG |
| | | AGAGAGGTGAACAAGAGAGTGGAGGACATCCACCCCACCGTGCCCAACCCCTACAACCT |
| | | GCTGAGCGGCCTGCCCCCCAGCCACCAGTGGTACACCGTGCTGGACCTGAAGGACGCCT |
| | | TCTTCTGCCTGAGACTGCACCCCACCAGCCAGCCCCTGTTCGCCTTCGAGTGGAGAGAC |
| | | CCCGAGATGGGCATCAGCGGCCAGCTGACCTGGACCAGACTGCCCCAGGGCTTCAAGAA |
| | | CAGCCCCACCCTGTTCAACGAGGCCCTGCACAGAGACCTGGCCGACTTCAGAATCCAGC |
| | | ACCCCGACCTGATCCTGCTGCAGTACGTGGACGACCTGCTGCTGGCCGCCACCAGCGAG |
| | | CTGGACTGCCAGCAGGGCACCAGAGCCCTGCTGCAGACCCTGGGCAACCTGGGCTACAG |
| | | AGCCAGCGCCAAGAAGGCCCAGATCTGCCAGAAGCAGGTGAAGTACCTGGGCTACCTGC |
| | | TGAAGGAGGGCCAGAGATGGCTGACCGAGGCCAGAAAGGAGACCGTGATGGGCCAGCCC |
| | | ACCCCCAAGACCCCCAGACAGCTGAGAGAGTTCCTGGGCAAGGCCGGCTTCTGCAGACT |
| | | GTTCATCCCCGGCTTCGCCGAGATGGCCGCCCCCCTGTACCCCCTGACCAAGCCCGGCA |
| | | CCCTGTTCAACTGGGGCCCCGACCAGCAGAAGGCCTACCAGGAGATCAAGCAGGCCCTG |
| | | CTGACCGCCCCCGCCCTGGGCCTGCCCGACCTGACCAAGCCCTTCGAGCTGTTCGTGGA |
| | | CGAGAAGCAGGGCTACGCCAAGGGCGTGCTGACCCAGAAGCTGGGCCCCTGGAGAAGAC |
| | | CCGTGGCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATGGGCCA |
| | | GCCCCTGGTGATCCTGGCCCCCCACGCCGTGGAGGCCCTGGTGAAGCAGCCCCCCGACA |
| | | GATGGCTGAGCAACGCCAGAATGACCCACTACCAGGCCCTGCTGCTGGACACCGACAGA |
| | | GTGCAGTTCGGCCCCGTGGTGGCCCTGAACCCCGCCACCCTGCTGCCCCTGCCCGAGGA |
| | | GGGCCTGCAGCACAACTGCCTGGACATCCTGGCCGAGGCCCACGGCACCAGACCCGACC |
| | | TGACCGACCAGCCCCTGCCCGACGCCGACCACACCTGGTACACCGACGGCAGCAGCCTG |
| | | CTGCAGGAGGGCCAGAGAAAGGCCGGCGCCGCCGTGACCACCGAGACCGAGGTGATCTG |
| | | GGCCAAGGCCCTGCCCGCCGGCACCAGCGCCCAGAGAGCCGAGCTGATCGCCCTGACCC |
| | | AGGCCCTGAAGATGGCCGAGGGCAAGAAGCTGAACGTGTACACCGACAGCAGATACGCC |
| | | TTCGCCACCGCCCACATCCACGGCGAGATCTACAGAAGAAGAGGCTGGCTGACCAGCGA |
| | | GGGCAAGGAGATCAAGAACAAGGACGAGATCCTGGCCCTGCTGAAGGCCCTGTTCCTGC |
| | | CCAAGAGACTGAGCATCATCCACTGCCCCGGCCACCAGAAGGGCCACAGCGCCGAGGCC |
| | | AGAGGCAACAGAATGGCCGACCAGGCCGCCAGAAAGGCCGCCATCACCGAGACCCCCGA |
| | | CACCAGCACCCTGCTGATCGAGAACAGCAGCCCCAGCGGCGGCAGCAAGAGAACCGCCG |
| | | ACGGCAGCGAGTTCGAGCCCAAGAAGAAGAGAAAGGTGggcggcagcAAGGACAAAAAG |
| | | AGCGACGGCAAGGACTCGCAAAAAAAAGGCAGCGGCGCCACCAACTTCAGCCTGCTGAA |
| | | GCAGGCCGGCGACGTGGAGGAGAACCCCGGCCCCcctgctgctaagagagtgaaactgg |
| | | atggcggcagcAAGGACAAGAAATCCGATGGCAAGGACAGCCAGAAAAAAGGCAGCAGA |
| | | GCCCTGGTGGTGATCAGACTGAGCAGAGTGACCGACGCCACCACCAGCCCCGAGAGACA |
| | | GCTGGAGAGCTGCCAGCAGCTGTGCGCCCAGAGAGGCTGGGACGTGGTGGGCGTGGCCG |
| | | AGGACCTGGACGTGAGCGGCGCCGTGGACCCCTTCGACAGAAAGAGAAGACCCAACCTG |
| | | GCCAGATGGCTGGCCTTCGAGGAGCAGCCCTTCGACGTGATCGTGGCCTACAGAGTGGA |
| | | CAGACTGACCAGAAGCATCAGACACCTGCAGCAGCTGGTGCACTGGGCCGAGGACCACA |
| | | AGAAGCTGGTGGTGAGCGCCACCGAGGCCCACTTCGACACCACCACCCCCTTCGCCGCC |
| | | GTGGTGATCGCCCTGATGGGCACCGTGGCCCAGATGGAGCTGGAGGCCATCAAGGAGAG |
| | | AAACAGAAGCGCCGCCCACTTCAACATCAGAGCCGGCAAGTACAGAGGCAGCCTGCCCC |
| | | CCTGGGGCTACCTGCCCACCAGAGTGGACGGCGAGTGGAGACTGGTGCCCGACCCCGTG |
| | | CAGAGAGAGAGAATCCTGGAGGTGTACCACAGAGTGGTGGACAACCACGAGCCCCTGCA |
| | | CCTGGTGGCCCACGACCTGAACAGAAGAGGCGTGCTGAGCCCCAAGGACTACTTCGCCC |
| | | AGCTGCAGGGCAGAGAGCCCCAGGGCAGAGAGTGGAGCGCCACCGCCCTGAAGAGAAGC |
| | | ATGATCAGCGAGGCCATGCTGGGCTACGCCACCCTGAACGGCAAGACCGTGAGAGACGA |
| | | CGACGGCGCCCCCCTGGTGAGAGCCGAGCCCATCCTGACCAGAGAGCAGCTGGAGGCCC |
| | | TGAGAGCCGAGCTGGTGAAGACCAGCAGAGCCAAGCCCGCCGTGAGCACCCCCAGCCTG |
| | | CTGCTGAGAGTGCTGTTCTGCGCCGTGTGCGGCGAGCCCGCCTACAAGTTCGCCGGCGG |
| | | CGGCAGAAAGCACCCCAGATACAGATGCAGAAGCATGGGCTTCCCCAAGCACTGCGGCA |
| | | ACGGCACCGTGGCCATGGCCGAGTGGGACGCCTTCTGCGAGGAGCAGGTGCTGGACCTG |
| | | CTGGGCGACGCCGAGAGACTGGAGAAGGTGTGGGTGGCCGGCAGCGACAGCGCCGTGGA |
| | | GCTGGCCGAGGTGAACGCCGAGCTGGTGGACCTGACCAGCCTGATCGGCAGCCCCGCCT |
| | | ACAGAGCCGGCAGCCCCCAGAGAGAGGCCCTGGACGCCAGAATCGCCGCCCTGGCCGCC |
| | | AGACAGGAGGAGCTGGAGGGCCTGGAGGCCAGACCCAGCGGCTGGGAGTGGAGAGAGAC |
| | | CGGCCAGAGATTCGGCGACTGGTGGAGAGAGCAGGACACCGCCGCCAAGAACACCTGGC |
| | | TGAGAAGCATGAACGTGAGACTGACCTTCGACGTGAGAGGCGGCCTGACCAGAACCATC |
| | | GACTTCGGCGACCTGCAGGAGTACGAGCAGCACCTGAGACTGGGCAGCGTGGTGGAGAG |
| | | ACTGCACACCGGCATGAGCGGCGGCAGCCCCAAGAAGAAGAGAAAGGTGTGA |
| 633 | PL1932 | MPAAKRVKLDGGSRALWIRLSRVTDATTSPERQLESCQQLCAQRGWDVVGVAEDLDVSG |
| | _AA | AVDPFDRKRRPNLARWLAFEEQPFDVIVAYRVDRLTRSIRHLQQLVHWAEDHKKLVVSA |
| | | TEAHFDTTTPFAAVVIALMGTVAQMELEAIKERNRSAAHFNIRAGKYRGSLPPWGYLPT |
| | | RVDGEWRLVPDPVQRERILEVYHRVVDNHEPLHLVAHDLNRRGVLSPKDYFAQLQGREP |
| | | QGREWSATALKRSMISEAMLGYATLNGKTVRDDDGAPLVRAEPILTREQLEALRAELVK |
| | | TSRAKPAVSTPSLLLRVLFCAVCGEPAYKFAGGGRKHPRYRCRSMGFPKHCGNGTVAMA |
| | | EWDAFCEEQVLDLLGDAERLEKVWVAGSDSAVELAEVNAELVDLTSLIGSPAYRAGSPQ |
| | | REALDARIAALAARQEELEGLEARPSGWEWRETGQRFGDWWREQDTAAKNTWLRSMNVR |
| | | LTFDVRGGLTRTIDFGDLQEYEQHLRLGSVVERLHTGMSGGSPKKKRKVGSGATNFSLL |
| | | KQAGDVEENPGPKRTADGSEFESPKKKRKVDKKYSIGLDIGTNSVGWAVITDEYKVPSK |
| | | KFKVLGNTDRHSIKKNLIGALLFDSGETAEATRLKRTARRRYTRRKNRICYLQEIFSNE |
| | | MAKVDDSFFHRLEESFLVEEDKKHERHPIFGNIVDEVAYHEKYPTIYHLRKKLVDSTDK |
| | | ADLRLIYLALAHMIKFRGHFLIEGDLNPDNSDVDKLFIQLVQTYNQLFEENPINASGVD |
| | | AKAILSARLSKSRRLENLIAQLPGEKKNGLFGNLIALSLGLTPNFKSNFDLAEDAKLQL |
| | | SKDTYDDDLDNLLAQIGDQYADLFLAAKNLSDAILLSDILRVNTEITKAPLSASMIKRY |
| | | DEHHQDLTLLKALVRQQLPEKYKEIFFDQSKNGYAGYIDGGASQEEFYKFIKPILEKMD |
| | | GTEELLVKLNREDLLRKQRTFDNGSIPHQIHLGELHAILRRQEDFYPFLKDNREKIEKI |
| | | LTFRIPYYVGPLARGNSRFAWMTRKSEETITPWNFEEVVDKGASAQSFIERMTNFDKNL |
| | | PNEKVLPKHSLLYEYFTVYNELTKVKYVTEGMRKPAFLSGEQKKAIVDLLFKTNRKVTV |
| | | KQLKEDYFKKIECFDSVEISGVEDRFNASLGTYHDLLKIIKDKDFLDNEENEDILEDIV |
| | | LTLTLFEDREMIEERLKTYAHLFDDKVMKQLKRRRYTGWGRLSRKLINGIRDKQSGKTI |
| | | LDFLKSDGFANRNFMQLIHDDSLTFKEDIQKAQVSGQGDSLHEHIANLAGSPAIKKGIL |
| | | QTVKVVDELVKVMGRHKPENIVIEMARENQTTQKGQKNSRERMKRIEEGIKELGSQILK |
| | | EHPVENTQLQNEKLYLYYLQNGRDMYVDQELDINRLSDYDVDAIVPQSFLKDDSIDNKV |
| | | LTRSDKNRGKSDNVPSEEVVKKMKNYWRQLLNAKLITQRKFDNLTKAERGGLSELDKAG |
| | | FIKRQLVETRQITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMIAKSEQEIGKAT |
| | | AKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLWVAKVEK |
| | | GKSKKLKSVKELLGITIMERSSFEKNPIDFLEAKGYKEVKKDLIIKLPKYSLFELENGR |
| | | KRMLASAGELQKGNELALPSKYVNFLYLASHYEKLKGSPEDNEQKQLFVEQHKHYLDEI |
| | | IEQISEFSKRVILADANLDKVLSAYNKHRDKPIREQAENIIHLFTLTNLGAPAAFKYFD |
| | | TTIDRKRYTSTKEVLDATLIHQSITGLYETRIDLSQLGGDSGGSSGGSSGSETPGTSES |
| | | ATPESSGGSSGGSSTLNIEDEYRLHETSKEPDVSLGSTWLSDFPQAWAETGGMGLAVRQ |
| | | APLIIPLKATSTPVSIKQYPMSQEARLGIKPHIQRLLDQGILVPCQSPWNTPLLPVKKP |
| | | GTNDYRPVQDLREVNKRVEDIHPTVPNPYNLLSGLPPSHQWYTVLDLKDAFFCLRLHPT |
| | | SQPLFAFEWRDPEMGISGQLTWTRLPQGFKNSPTLFNEALHRDLADFRIQHPDLILLQY |
| | | VDDLLLAATSELDCQQGTRALLQTLGNLGYRASAKKAQICQKQVKYLGYLLKEGQRWLT |
| | | EARKETVMGQPTPKTPRQLREFLGKAGFCRLFIPGFAEMAAPLYPLTKPGTLFNWGPDQ |
| | | QKAYQEIKQALLTAPALGLPDLTKPFELFVDEKQGYAKGVLTQKLGPWRRPVAYLSKKL |
| | | DPVAAGWPPCLRMVAAIAVLTKDAGKLTMGQPLVILAPHAVEALVKQPPDRWLSNARMT |
| | | HYQALLLDTDRVQFGPVVALNPATLLPLPEEGLQHNCLDILAEAHGTRPDLTDQPLPDA |
| | | DHTWYTDGSSLLQEGQRKAGAAVTTETEVIWAKALPAGTSAQRAELIALTQALKMAEGK |
| | | KLNVYTDSRYAFATAHIHGEIYRRRGWLTSEGKEIKNKDEILALLKALFLPKRLSIIHC |
| | | PGHQKGHSAEARGNRMADQAARKAAITETPDTSTLLIENSSPSGGSKRTADGSEFEPKK |
| | | KRKV* |
| 634 | PL1932_ | ATGcctgctgctaagagagtgaaactggatggcggcagcAGAGCCCTGGTGGTGATCAG |
| | DNA | ACTGAGCAGAGTGACCGACGCCACCACCAGCCCCGAGAGACAGCTGGAGAGCTGCCAGC |
| | | AGCTGTGCGCCCAGAGAGGCTGGGACGTGGTGGGCGTGGCCGAGGACCTGGACGTGAGC |
| | | GGCGCCGTGGACCCCTTCGACAGAAAGAGAAGACCCAACCTGGCCAGATGGCTGGCCTT |
| | | CGAGGAGCAGCCCTTCGACGTGATCGTGGCCTACAGAGTGGACAGACTGACCAGAAGCA |
| | | TCAGACACCTGCAGCAGCTGGTGCACTGGGCCGAGGACCACAAGAAGCTGGTGGTGAGC |
| | | GCCACCGAGGCCCACTTCGACACCACCACCCCCTTCGCCGCCGTGGTGATCGCCCTGAT |
| | | GGGCACCGTGGCCCAGATGGAGCTGGAGGCCATCAAGGAGAGAAACAGAAGCGCCGCCC |
| | | ACTTCAACATCAGAGCCGGCAAGTACAGAGGCAGCCTGCCCCCCTGGGGCTACCTGCCC |
| | | ACCAGAGTGGACGGCGAGTGGAGACTGGTGCCCGACCCCGTGCAGAGAGAGAGAATCCT |
| | | GGAGGTGTACCACAGAGTGGTGGACAACCACGAGCCCCTGCACCTGGTGGCCCACGACC |
| | | TGAACAGAAGAGGCGTGCTGAGCCCCAAGGACTACTTCGCCCAGCTGCAGGGCAGAGAG |
| | | CCCCAGGGCAGAGAGTGGAGCGCCACCGCCCTGAAGAGAAGCATGATCAGCGAGGCCAT |
| | | GCTGGGCTACGCCACCCTGAACGGCAAGACCGTGAGAGACGACGACGGCGCCCCCCTGG |
| | | TGAGAGCCGAGCCCATCCTGACCAGAGAGCAGCTGGAGGCCCTGAGAGCCGAGCTGGTG |
| | | AAGACCAGCAGAGCCAAGCCCGCCGTGAGCACCCCCAGCCTGCTGCTGAGAGTGCTGTT |
| | | CTGCGCCGTGTGCGGCGAGCCCGCCTACAAGTTCGCCGGCGGCGGCAGAAAGCACCCCA |
| | | GATACAGATGCAGAAGCATGGGCTTCCCCAAGCACTGCGGCAACGGCACCGTGGCCATG |
| | | GCCGAGTGGGACGCCTTCTGCGAGGAGCAGGTGCTGGACCTGCTGGGCGACGCCGAGAG |
| | | ACTGGAGAAGGTGTGGGTGGCCGGCAGCGACAGCGCCGTGGAGCTGGCCGAGGTGAACG |
| | | CCGAGCTGGTGGACCTGACCAGCCTGATCGGCAGCCCCGCCTACAGAGCCGGCAGCCCC |
| | | CAGAGAGAGGCCCTGGACGCCAGAATCGCCGCCCTGGCCGCCAGACAGGAGGAGCTGGA |
| | | GGGCCTGGAGGCCAGACCCAGCGGCTGGGAGTGGAGAGAGACCGGCCAGAGATTCGGCG |
| | | ACTGGTGGAGAGAGCAGGACACCGCCGCCAAGAACACCTGGCTGAGAAGCATGAACGTG |
| | | AGACTGACCTTCGACGTGAGAGGCGGCCTGACCAGAACCATCGACTTCGGCGACCTGCA |
| | | GGAGTACGAGCAGCACCTGAGACTGGGCAGCGTGGTGGAGAGACTGCACACCGGCATGA |
| | | GCGGCGGCAGCCCCAAGAAGAAGAGAAAGGTGGGCAGCGGCGCCACCAACTTCAGCCTG |
| | | CTGAAGCAGGCCGGCGACGTGGAGGAGAACCCCGGCCCCAAGAGAACCGCCGACGGCAG |
| | | CGAGTTCGAGAGCCCCAAGAAGAAGAGAAAGGTGGACAAGAAGTACAGCATCGGCCTGG |
| | | ACATCGGCACCAACAGCGTGGGCTGGGCCGTGATCACCGACGAGTACAAGGTGCCCAGC |
| | | AAGAAGTTCAAGGTGCTGGGCAACACCGACAGACACAGCATCAAGAAGAACCTGATCGG |
| | | CGCCCTGCTGTTCGACAGCGGCGAGACCGCCGAGGCCACCAGACTGAAGAGAACCGCCA |
| | | GAAGAAGATACACCAGAAGAAAGAACAGAATCTGCTACCTGCAGGAGATCTTCAGCAAC |
| | | GAGATGGCCAAGGTGGACGACAGCTTCTTCCACAGACTGGAGGAGAGCTTCCTGGTGGA |
| | | GGAGGACAAGAAGCACGAGAGACACCCCATCTTCGGCAACATCGTGGACGAGGTGGCCT |
| | | ACCACGAGAAGTACCCCACCATCTACCACCTGAGAAAGAAGCTGGTGGACAGCACCGAC |
| | | AAGGCCGACCTGAGACTGATCTACCTGGCCCTGGCCCACATGATCAAGTTCAGAGGCCA |
| | | CTTCCTGATCGAGGGCGACCTGAACCCCGACAACAGCGACGTGGACAAGCTGTTCATCC |
| | | AGCTGGTGCAGACCTACAACCAGCTGTTCGAGGAGAACCCCATCAACGCCAGCGGCGTG |
| | | GACGCCAAGGCCATCCTGAGCGCCAGACTGAGCAAGAGCAGAAGACTGGAGAACCTGAT |
| | | CGCCCAGCTGCCCGGCGAGAAGAAGAACGGCCTGTTCGGCAACCTGATCGCCCTGAGCC |
| | | TGGGCCTGACCCCCAACTTCAAGAGCAACTTCGACCTGGCCGAGGACGCCAAGCTGCAG |
| | | CTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACA |
| | | TCCTGAGAGTGAACACCGAGATCACCAAGGCCCCCCTGAGCGCCAGCATGATCAAGAGA |
| | | TACGACGAGCACCACCAGGACCTGACCCTGCTGAAGGCCCTGGTGAGACAGCAGCTGCC |
| | | CGAGAAGTACAAGGAGATCTTCTTCGACCAGAGCAAGAACGGCTACGCCGGCTACATCG |
| | | ACGGCGGCGCCAGCCAGGAGGAGTTCTACAAGTTCATCAAGCCCATCCTGGAGAAGATG |
| | | GACGGCACCGAGGAGCTGCTGGTGAAGCTGAACAGAGAGGACCTGCTGAGAAAGCAGAG |
| | | AACCTTCGACAACGGCAGCATCCCCCACCAGATCCACCTGGGCGAGCTGCACGCCATCC |
| | | TGAGAAGACAGGAGGACTTCTACCCCTTCCTGAAGGACAACAGAGAGAAGATCGAGAAG |
| | | ATCCTGACCTTCAGAATCCCCTACTACGTGGGCCCCCTGGCCAGAGGCAACAGCAGATT |
| | | CGCCTGGATGACCAGAAAGAGCGAGGAGACCATCACCCCCTGGAACTTCGAGGAGGTGG |
| | | TGGACAAGGGCGCCAGCGCCCAGAGCTTCATCGAGAGAATGACCAACTTCGACAAGAAC |
| | | CTGCCCAACGAGAAGGTGCTGCCCAAGCACAGCCTGCTGTACGAGTACTTCACCGTGTA |
| | | CAACGAGCTGACCAAGGTGAAGTACGTGACCGAGGGCATGAGAAAGCCCGCCTTCCTGA |
| | | GCGGCGAGCAGAAGAAGGCCATCGTGGACCTGCTGTTCAAGACCAACAGAAAGGTGACC |
| | | GTGAAGCAGCTGAAGGAGGACTACTTCAAGAAGATCGAGTGCTTCGACAGCGTGGAGAT |
| | | CAGCGGCGTGGAGGACAGATTCAACGCCAGCCTGGGCACCTACCACGACCTGCTGAAGA |
| | | TCATCAAGGACAAGGACTTCCTGGACAACGAGGAGAACGAGGACATCCTGGAGGACATC |
| | | GTGCTGACCCTGACCCTGTTCGAGGACAGAGAGATGATCGAGGAGAGACTGAAGACCTA |
| | | CGCCCACCTGTTCGACGACAAGGTGATGAAGCAGCTGAAGAGAAGAAGATACACCGGCT |
| | | GGGGCAGACTGAGCAGAAAGCTGATCAACGGCATCAGAGACAAGCAGAGCGGCAAGACC |
| | | ATCCTGGACTTCCTGAAGAGTGACGGCTTCGCCAACAGAAACTTCATGCAGCTGATCCA |
| | | CGACGACAGCCTGACCTTCAAGGAGGACATCCAGAAGGCCCAGGTGAGCGGCCAGGGCG |
| | | ACAGCCTGCACGAGCACATCGCCAACCTGGCCGGCAGCCCCGCCATCAAGAAGGGCATC |
| | | CTGCAGACCGTGAAGGTGGTGGACGAGCTGGTGAAGGTGATGGGCAGACACAAGCCCGA |
| | | GAACATCGTGATCGAGATGGCCAGAGAGAACCAGACCACCCAGAAGGGCCAGAAGAACA |
| | | GCAGAGAGAGAATGAAGAGAATCGAGGAGGGCATCAAGGAGCTGGGCAGCCAGATCCTG |
| | | AAGGAGCACCCCGTGGAGAACACCCAGCTGCAGAACGAGAAGCTGTACCTGTACTACCT |
| | | GCAGAACGGCAGAGACATGTACGTGGACCAGGAGCTGGACATCAACAGACTGAGCGACT |
| | | ACGACGTGGACGCCATCGTGCCCCAGAGCTTCCTGAAGGACGACAGCATCGACAACAAG |
| | | GTGCTGACCAGAAGCGACAAGAACAGAGGCAAGAGCGACAACGTGCCCAGCGAGGAGGT |
| | | GGTGAAGAAGATGAAGAACTACTGGAGACAGCTGCTGAACGCCAAGCTGATCACCCAGA |
| | | GAAAGTTCGACAACCTGACCAAGGCCGAGAGAGGCGGCCTGAGCGAGCTGGACAAGGCC |
| | | GGCTTCATCAAGAGACAGCTGGTGGAGACCAGACAGATCACCAAGCACGTGGCCCAGAT |
| | | CCTGGACAGCAGAATGAACACCAAGTACGACGAGAACGACAAGCTGATCAGAGAGGTGA |
| | | AGGTGATCACCCTGAAGAGTAAGCTGGTGAGCGACTTCAGAAAGGACTTCCAGTTCTAC |
| | | AAGGTGAGAGAGATCAACAACTACCACCACGCCCACGACGCCTACCTGAACGCCGTGGT |
| | | GGGCACCGCCCTGATCAAGAAGTACCCCAAGCTGGAGAGCGAGTTCGTGTACGGCGACT |
| | | ACAAGGTGTACGACGTGAGAAAGATGATCGCCAAGAGCGAGCAGGAGATCGGCAAGGCC |
| | | ACCGCCAAGTACTTCTTCTACAGCAACATCATGAACTTCTTCAAGACCGAGATCACCCT |
| | | GGCCAACGGCGAGATCAGAAAGAGACCCCTGATCGAGACCAACGGCGAGACCGGCGAGA |
| | | TCGTGTGGGACAAGGGCAGAGACTTCGCCACCGTGAGAAAGGTGCTGAGCATGCCCCAG |
| | | GTGAACATCGTGAAGAAGACCGAGGTGCAGACCGGCGGCTTCAGCAAGGAGAGCATCCT |
| | | GCCCAAGAGAAACAGCGACAAGCTGATCGCCAGAAAGAAGGACTGGGACCCCAAGAAGT |
| | | ACGGCGGCTTCGACAGCCCCACCGTGGCCTACAGCGTGCTGGTGGTGGCCAAGGTGGAG |
| | | AAGGGCAAGAGCAAGAAGCTGAAGAGTGTGAAGGAGCTGCTGGGCATCACCATCATGGA |
| | | GAGAAGCAGCTTCGAGAAGAACCCCATCGACTTCCTGGAGGCCAAGGGCTACAAGGAGG |
| | | TGAAGAAGGACCTGATCATCAAGCTGCCCAAGTACAGCCTGTTCGAGCTGGAGAACGGC |
| | | AGAAAGAGAATGCTGGCCAGCGCCGGCGAGCTGCAGAAGGGCAACGAGCTGGCCCTGCC |
| | | CAGCAAGTACGTGAACTTCCTGTACCTGGCCAGCCACTACGAGAAGCTGAAGGGCAGCC |
| | | CCGAGGACAACGAGCAGAAGCAGCTGTTCGTGGAGCAGCACAAGCACTACCTGGACGAG |
| | | ATCATCGAGCAGATCAGCGAGTTCAGCAAGAGAGTGATCCTGGCCGACGCCAACCTGGA |
| | | CAAGGTGCTGAGCGCCTACAACAAGCACAGAGACAAGCCCATCAGAGAGCAGGCCGAGA |
| | | ACATCATCCACCTGTTCACCCTGACCAACCTGGGCGCCCCCGCCGCCTTCAAGTACTTC |
| | | GACACCACCATCGACAGAAAGAGATACACCAGCACCAAGGAGGTGCTGGACGCCACCCT |
| | | GATCCACCAGAGCATCACCGGCCTGTACGAGACCAGAATCGACCTGAGCCAGCTGGGCG |
| | | GCGACAGCGGCGGCAGCAGCGGCGGCAGCAGCGGCAGCGAGACCCCCGGCACCAGCGAG |
| | | AGCGCCACCCCCGAGAGCAGCGGCGGCAGCAGCGGCGGCAGCAGCACCCTGAACATCGA |
| | | GGACGAGTACAGACTGCACGAGACCAGCAAGGAGCCCGACGTGAGCCTGGGCAGCACCT |
| | | GGCTGAGCGACTTCCCCCAGGCCTGGGCCGAGACCGGCGGCATGGGCCTGGCCGTGAGA |
| | | CAGGCCCCCCTGATCATCCCCCTGAAGGCCACCAGCACCCCCGTGAGCATCAAGCAGTA |
| | | CCCCATGAGCCAGGAGGCCAGACTGGGCATCAAGCCCCACATCCAGAGACTGCTGGACC |
| | | AGGGCATCCTGGTGCCCTGCCAGAGCCCCTGGAACACCCCCCTGCTGCCCGTGAAGAAG |
| | | CCCGGCACCAACGACTACAGACCCGTGCAGGACCTGAGAGAGGTGAACAAGAGAGTGGA |
| | | GGACATCCACCCCACCGTGCCCAACCCCTACAACCTGCTGAGCGGCCTGCCCCCCAGCC |
| | | ACCAGTGGTACACCGTGCTGGACCTGAAGGACGCCTTCTTCTGCCTGAGACTGCACCCC |
| | | ACCAGCCAGCCCCTGTTCGCCTTCGAGTGGAGAGACCCCGAGATGGGCATCAGCGGCCA |
| | | GCTGACCTGGACCAGACTGCCCCAGGGCTTCAAGAACAGCCCCACCCTGTTCAACGAGG |
| | | CCCTGCACAGAGACCTGGCCGACTTCAGAATCCAGCACCCCGACCTGATCCTGCTGCAG |
| | | TACGTGGACGACCTGCTGCTGGCCGCCACCAGCGAGCTGGACTGCCAGCAGGGCACCAG |
| | | AGCCCTGCTGCAGACCCTGGGCAACCTGGGCTACAGAGCCAGCGCCAAGAAGGCCCAGA |
| | | TCTGCCAGAAGCAGGTGAAGTACCTGGGCTACCTGCTGAAGGAGGGCCAGAGATGGCTG |
| | | ACCGAGGCCAGAAAGGAGACCGTGATGGGCCAGCCCACCCCCAAGACCCCCAGACAGCT |
| | | GAGAGAGTTCCTGGGCAAGGCCGGCTTCTGCAGACTGTTCATCCCCGGCTTCGCCGAGA |
| | | TGGCCGCCCCCCTGTACCCCCTGACCAAGCCCGGCACCCTGTTCAACTGGGGCCCCGAC |
| | | CAGCAGAAGGCCTACCAGGAGATCAAGCAGGCCCTGCTGACCGCCCCCGCCCTGGGCCT |
| | | GCCCGACCTGACCAAGCCCTTCGAGCTGTTCGTGGACGAGAAGCAGGGCTACGCCAAGG |
| | | GCGTGCTGACCCAGAAGCTGGGCCCCTGGAGAAGACCCGTGGCCTACCTGAGCAAGAAG |
| | | CTGGACCCCGTGGCCGCCGGCTGGCCCCCCTGCCTGAGAATGGTGGCCGCCATCGCCGT |
| | | GCTGACCAAGGACGCCGGCAAGCTGACCATGGGCCAGCCCCTGGTGATCCTGGCCCCCC |
| | | ACGCCGTGGAGGCCCTGGTGAAGCAGCCCCCCGACAGATGGCTGAGCAACGCCAGAATG |
| | | ACCCACTACCAGGCCCTGCTGCTGGACACCGACAGAGTGCAGTTCGGCCCCGTGGTGGC |
| | | CCTGAACCCCGCCACCCTGCTGCCCCTGCCCGAGGAGGGCCTGCAGCACAACTGCCTGG |
| | | ACATCCTGGCCGAGGCCCACGGCACCAGACCCGACCTGACCGACCAGCCCCTGCCCGAC |
| | | GCCGACCACACCTGGTACACCGACGGCAGCAGCCTGCTGCAGGAGGGCCAGAGAAAGGC |
| | | CGGCGCCGCCGTGACCACCGAGACCGAGGTGATCTGGGCCAAGGCCCTGCCCGCCGGCA |
| | | CCAGCGCCCAGAGAGCCGAGCTGATCGCCCTGACCCAGGCCCTGAAGATGGCCGAGGGC |
| | | AAGAAGCTGAACGTGTACACCGACAGCAGATACGCCTTCGCCACCGCCCACATCCACGG |
| | | CGAGATCTACAGAAGAAGAGGCTGGCTGACCAGCGAGGGCAAGGAGATCAAGAACAAGG |
| | | ACGAGATCCTGGCCCTGCTGAAGGCCCTGTTCCTGCCCAAGAGACTGAGCATCATCCAC |
| | | TGCCCCGGCCACCAGAAGGGCCACAGCGCCGAGGCCAGAGGCAACAGAATGGCCGACCA |
| | | GGCCGCCAGAAAGGCCGCCATCACCGAGACCCCCGACACCAGCACCCTGCTGATCGAGA |
| | | ACAGCAGCCCCAGCGGCGGCAGCAAGAGAACCGCCGACGGCAGCGAGTTCGAGCCCAAG |
| | | AAGAAGAGAAAGGTGTGA |
| 635 | PL1933 | MPAAKRVKLDGGSRALWVIRLSRVTDATTSPERQLESCQQLCAQRGWDVVGVAEDLDVS |
| | _AA | GAVDPFDRKRRPNLARWLAFEEQPFDVIVAYRVDRLTRSIRHLQQLVHWAEDHKKLVVS |
| | | ATEAHFDTTTPFAAVVIALMGTVAQMELEAIKERNRSAAHFNIRAGKYRGSLPPWGYLP |
| | | TRVDGEWRLVPDPVQRERILEVYHRWVDNHEPLHLVAHDLNRRGVLSPKDYFAQLQGRE |
| | | PQGREWSATALKRSMISEAMLGYATLNGKTVRDDDGAPLVRAEPILTREQLEALRAELV |
| | | KTSRAKPAVSTPSLLLRVLFCAVCGEPAYKFAGGGRKHPRYRCRSMGFPKHCGNGTVAM |
| | | AEWDAFCEEQVLDLLGDAERLEKVWVAGSDSAVELAEVNAELVDLTSLIGSPAYRAGSP |
| | | QREALDARIAALAARQEELEGLEARPSGWEWRETGQRFGDWWREQDTAAKNTWLRSMNV |
| | | RLTFDVRGGLTRTIDFGDLQEYEQHLRLGSVVERLHTGMSGGSPKKKRKVGSGATNFSL |
| | | LKQAGDVEENPGPKRTADGSEFESPKKKRKVDKKYSIGLDIGTNSVGWAVITDEYKVPS |
| | | KKFKVLGNTDRHSIKKNLIGALLFDSGETAEATRLKRTARRRYTRRKNRICYLQEIFSN |
| | | EMAKVDDSFFHRLEESFLVEEDKKHERHPIFGNIVDEVAYHEKYPTIYHLRKKLVDSTD |
| | | KADLRLIYLALAHMIKFRGHFLIEGDLNPDNSDVDKLFIQLVQTYNQLFEENPINASGV |
| | | DAKAILSARLSKSRRLENLIAQLPGEKKNGLFGNLIALSLGLTPNFKSNFDLAEDAKLQ |
| | | LSKDTYDDDLDNLLAQIGDQYADLFLAAKNLSDAILLSDILRVNTEITKAPLSASMIKR |
| | | YDEHHQDLTLLKALVRQQLPEKYKEIFFDQSKNGYAGYIDGGASQEEFYKFIKPILEKM |
| | | DGTEELLVKLNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKAIVDLLFKTNRKVT |
| | | VKQLKEDYFKKIECFDSVEISGVEDRFNASLGTYHDLLKIIKDKDFLDNEENEDILEDI |
| | | VLTLTLFEDREMIEERLKTYAHLFDDKVMKQLKRRRYTGWGRLSRKLINGIRDKQSGKT |
| | | ILDFLKSDGFANRNFMQLIHDDSLTFKEDIQKAQVSGQGDSLHEHIANLAGSPAIKKGI |
| | | LQTVKVVDELVKVMGRHKPENIVIEMARENQTTQKGQKNSRERMKRIEEGIKELGSQIL |
| | | KEHPVENTQLQNEKLYLYYLQNGRDMYVDQELDINRLSDYDVDAIVPQSFLKDDSIDNK |
| | | VLTRSDKNRGKSDNVPSEEVVKKMKNYWRQLLNAKLITQRKFDNLTKAERGGLSELDKA |
| | | GFIKRQLVETRQITKHVAQILDSRMNTKYDENDKLIREVKVITLKSKLVSDFRKDFQFY |
| | | KVREINNYHHAHDAYLNAVVGTALIKKYPKLESEFVYGDYKVYDVRKMIAKSEQEIGKA |
| | | TAKYFFYSNIMNFFKTEITLANGEIRKRPLIETNGETGEIVWDKGRDFATVRKVLSMPQ |
| | | VNIVKKTEVQTGGFSKESILPKRNSDKLIARKKDWDPKKYGGFDSPTVAYSVLVVAKVE |
| | | KGKSKKLKSVKELLGITIMERSSFEKNPIDFLEAKGYKEVKKDLIIKLPKYSLFELENG |
| | | RKRMLASAGELQKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLGAPAAFKYF |
| | | DTTIDRKRYTSTKEVLDATLIHQSITGLYETRIDLSQLGGDSGGSSGGSSGSETPGTSE |
| | | SATPESSGGSSGGSSTLNIEDEYRLHETSKEPDVSLGSTWLSDFPQAWAETGGMGLAVR |
| | | QAPLIIPLKATSTPVSIKQYPMSQEARLGIKPHIQRLLDQGILVPCQSPWNTPLLPVKK |
| | | PGTNDYRPVQDLREVNKRVEDIHPTVPNPYNLLSGLPPSHQWYTVLDLKDAFFCLRLHP |
| | | TSQPLFAFEWRDPEMGISGQLTWTRLPQGFKNSPTLFNEALHRDLADFRIQHPDLILLQ |
| | | YVDDLLLAATSELDCQQGTRALLQTLGNLGYRASAKKAQICQKQVKYLGYLLKEGQRWL |
| | | TEARKETVMGQPTPKTPRQLREFLGKAGFCRLFIPGFAEMAAPLYPLTKPGTLFNWGPD |
| | | QQKAYQEIKQALLTAPALGLPDLTKPFELFVDEKQGYAKGVLTQKLGPWRRPVAYLSKK |
| | | LDPVAAGWPPCLRMVAAIAVLTKDAGKLTMGQPLVILAPHAVEALVKQPPDRWLSNARM |
| | | THYQALLLDTDRVQFGPVVALNPATLLPLPEEGLQHNCLDILAEAHGTRPDLTDQPLPD |
| | | ADHTWYTDGSSLLQEGQRKAGAAVTTETEVIWAKALPAGTSAQRAELIALTQALKMAEG |
| | | KKLNVYTDSRYAFATAHIHGEIYRRRGWLTSEGKEIKNKDEILALLKALFLPKRLSIIH |
| | | CPGHQKGHSAEARGNRMADQAARKAAITETPDTSTLLIENSSPSGGSKRTADGSEFEPK |
| | | KKRKV* |
| 636 | PL1933_ | ATGcctgctgctaagagagtgaaactggatggcggcagcAGAGCCCTGGTGGTGATCAG |
| | DNA | ACTGAGCAGAGTGACCGACGCCACCACCAGCCCCGAGAGACAGCTGGAGAGCTGCCAGC |
| | | AGCTGTGCGCCCAGAGAGGCTGGGACGTGGTGGGCGTGGCCGAGGACCTGGACGTGAGC |
| | | GGCGCCGTGGACCCCTTCGACAGAAAGAGAAGACCCAACCTGGCCAGATGGCTGGCCTT |
| | | CGAGGAGCAGCCCTTCGACGTGATCGTGGCCTACAGAGTGGACAGACTGACCAGAAGCA |
| | | TCAGACACCTGCAGCAGCTGGTGCACTGGGCCGAGGACCACAAGAAGCTGGTGGTGAGC |
| | | GCCACCGAGGCCCACTTCGACACCACCACCCCCTTCGCCGCCGTGGTGATCGCCCTGAT |
| | | GGGCACCGTGGCCCAGATGGAGCTGGAGGCCATCAAGGAGAGAAACAGAAGCGCCGCCC |
| | | ACTTCAACATCAGAGCCGGCAAGTACAGAGGCAGCCTGCCCCCCTGGGGCTACCTGCCC |
| | | ACCAGAGTGGACGGCGAGTGGAGACTGGTGCCCGACCCCGTGCAGAGAGAGAGAATCCT |
| | | GGAGGTGTACCACAGAGTGGTGGACAACCACGAGCCCCTGCACCTGGTGGCCCACGACC |
| | | TGAACAGAAGAGGCGTGCTGAGCCCCAAGGACTACTTCGCCCAGCTGCAGGGCAGAGAG |
| | | CCCCAGGGCAGAGAGTGGAGCGCCACCGCCCTGAAGAGAAGCATGATCAGCGAGGCCAT |
| | | GCTGGGCTACGCCACCCTGAACGGCAAGACCGTGAGAGACGACGACGGCGCCCCCCTGG |
| | | TGAGAGCCGAGCCCATCCTGACCAGAGAGCAGCTGGAGGCCCTGAGAGCCGAGCTGGTG |
| | | AAGACCAGCAGAGCCAAGCCCGCCGTGAGCACCCCCAGCCTGCTGCTGAGAGTGCTGTT |
| | | CTGCGCCGTGTGCGGCGAGCCCGCCTACAAGTTCGCCGGGGGGGGCAGAAAGCACCCCA |
| | | GATACAGATGCAGAAGCATGGGCTTCCCCAAGCACTGCGGCAACGGCACCGTGGCCATG |
| | | GCCGAGTGGGACGCCTTCTGCGAGGAGCAGGTGCTGGACCTGCTGGGCGACGCCGAGAG |
| | | ACTGGAGAAGGTGTGGGTGGCCGGCAGCGACAGCGCCGTGGAGCTGGCCGAGGTGAACG |
| | | CCGAGCTGGTGGACCTGACCAGCCTGATCGGCAGCCCCGCCTACAGAGCCGGCAGCCCC |
| | | CAGAGAGAGGCCCTGGACGCCAGAATCGCCGCCCTGGCCGCCAGACAGGAGGAGCTGGA |
| | | GGGCCTGGAGGCCAGACCCAGCGGCTGGGAGTGGAGAGAGACCGGCCAGAGATTCGGCG |
| | | ACTGGTGGAGAGAGCAGGACACCGCCGCCAAGAACACCTGGCTGAGAAGCATGAACGTG |
| | | AGACTGACCTTCGACGTGAGAGGCGGCCTGACCAGAACCATCGACTTCGGCGACCTGCA |
| | | GGAGTACGAGCAGCACCTGAGACTGGGCAGCGTGGTGGAGAGACTGCACACCGGCATGA |
| | | GCGGCGGCAGCCCCAAGAAGAAGAGAAAGGTGGGCAGCGGCGCCACCAACTTCAGCCTG |
| | | CTGAAGCAGGCCGGCGACGTGGAGGAGAACCCCGGCCCCAAGAGAACCGCCGACGGCAG |
| | | CGAGTTCGAGAGCCCCAAGAAGAAGAGAAAGGTGGACAAGAAGTACAGCATCGGCCTGG |
| | | ACATCGGCACCAACAGCGTGGGCTGGGCCGTGATCACCGACGAGTACAAGGTGCCCAGC |
| | | AAGAAGTTCAAGGTGCTGGGCAACACCGACAGACACAGCATCAAGAAGAACCTGATCGG |
| | | CGCCCTGCTGTTCGACAGCGGCGAGACCGCCGAGGCCACCAGACTGAAGAGAACCGCCA |
| | | GAAGAAGATACACCAGAAGAAAGAACAGAATCTGCTACCTGCAGGAGATCTTCAGCAAC |
| | | GAGATGGCCAAGGTGGACGACAGCTTCTTCCACAGACTGGAGGAGAGCTTCCTGGTGGA |
| | | GGAGGACAAGAAGCACGAGAGACACCCCATCTTCGGCAACATCGTGGACGAGGTGGCCT |
| | | ACCACGAGAAGTACCCCACCATCTACCACCTGAGAAAGAAGCTGGTGGACAGCACCGAC |
| | | AAGGCCGACCTGAGACTGATCTACCTGGCCCTGGCCCACATGATCAAGTTCAGAGGCCA |
| | | CTTCCTGATCGAGGGCGACCTGAACCCCGACAACAGCGACGTGGACAAGCTGTTCATCC |
| | | AGCTGGTGCAGACCTACAACCAGCTGTTCGAGGAGAACCCCATCAACGCCAGCGGCGTG |
| | | GACGCCAAGGCCATCCTGAGCGCCAGACTGAGCAAGAGCAGAAGACTGGAGAACCTGAT |
| | | CGCCCAGCTGCCCGGCGAGAAGAAGAACGGCCTGTTCGGCAACCTGATCGCCCTGAGCC |
| | | TGGGCCTGACCCCCAACTTCAAGAGCAACTTCGACCTGGCCGAGGACGCCAAGCTGCAG |
| | | CTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACA |
| | | TCCTGAGAGTGAACACCGAGATCACCAAGGCCCCCCTGAGCGCCAGCATGATCAAGAGA |
| | | TACGACGAGCACCACCAGGACCTGACCCTGCTGAAGGCCCTGGTGAGACAGCAGCTGCC |
| | | CGAGAAGTACAAGGAGATCTTCTTCGACCAGAGCAAGAACGGCTACGCCGGCTACATCG |
| | | ACGGCGGCGCCAGCCAGGAGGAGTTCTACAAGTTCATCAAGCCCATCCTGGAGAAGATG |
| | | GACGGCACCGAGGAGCTGCTGGTGAAGCTGAACAGAGAGGACCTGCTGAGAAAGCAGAG |
| | | AACCTTCGACAACGGCAGCATCCCCCACCAGATCCACCTGGGCGAGCTGCACGCCATCC |
| | | TGAGAAGACAGGAGGACTTCTACCCCTTCCTGAAGGACAACAGAGAGAAGATCGAGAAG |
| | | ATCCTGACCTTCAGAATCCCCTACTACGTGGGCCCCCTGGCCAGAGGCAACAGCAGATT |
| | | CGCCTGGATGACCAGAAAGAGCGAGGAGACCATCACCCCCTGGAACTTCGAGGAGGTGG |
| | | TGGACAAGGGCGCCAGCGCCCAGAGCTTCATCGAGAGAATGACCAACTTCGACAAGAAC |
| | | CTGCCCAACGAGAAGGTGCTGCCCAAGCACAGCCTGCTGTACGAGTACTTCACCGTGTA |
| | | CAACGAGCTGACCAAGGTGAAGTACGTGACCGAGGGCATGAGAAAGCCCGCCTTCCTGA |
| | | GCGGCGAGCAGAAGAAGGCCATCGTGGACCTGCTGTTCAAGACCAACAGAAAGGTGACC |
| | | GTGAAGCAGCTGAAGGAGGACTACTTCAAGAAGATCGAGTGCTTCGACAGCGTGGAGAT |
| | | CAGCGGCGTGGAGGACAGATTCAACGCCAGCCTGGGCACCTACCACGACCTGCTGAAGA |
| | | TCATCAAGGACAAGGACTTCCTGGACAACGAGGAGAACGAGGACATCCTGGAGGACATC |
| | | GTGCTGACCCTGACCCTGTTCGAGGACAGAGAGATGATCGAGGAGAGACTGAAGACCTA |
| | | CGCCCACCTGTTCGACGACAAGGTGATGAAGCAGCTGAAGAGAAGAAGATACACCGGCT |
| | | GGGGCAGACTGAGCAGAAAGCTGATCAACGGCATCAGAGACAAGCAGAGCGGCAAGACC |
| | | ATCCTGGACTTCCTGAAGAGTGACGGCTTCGCCAACAGAAACTTCATGCAGCTGATCCA |
| | | CGACGACAGCCTGACCTTCAAGGAGGACATCCAGAAGGCCCAGGTGAGCGGCCAGGGCG |
| | | ACAGCCTGCACGAGCACATCGCCAACCTGGCCGGCAGCCCCGCCATCAAGAAGGGCATC |
| | | CTGCAGACCGTGAAGGTGGTGGACGAGCTGGTGAAGGTGATGGGCAGACACAAGCCCGA |
| | | GAACATCGTGATCGAGATGGCCAGAGAGAACCAGACCACCCAGAAGGGCCAGAAGAACA |
| | | GCAGAGAGAGAATGAAGAGAATCGAGGAGGGCATCAAGGAGCTGGGCAGCCAGATCCTG |
| | | AAGGAGCACCCCGTGGAGAACACCCAGCTGCAGAACGAGAAGCTGTACCTGTACTACCT |
| | | GCAGAACGGCAGAGACATGTACGTGGACCAGGAGCTGGACATCAACAGACTGAGCGACT |
| | | ACGACGTGGACGCCATCGTGCCCCAGAGCTTCCTGAAGGACGACAGCATCGACAACAAG |
| | | GTGCTGACCAGAAGCGACAAGAACAGAGGCAAGAGCGACAACGTGCCCAGCGAGGAGGT |
| | | GGTGAAGAAGATGAAGAACTACTGGAGACAGCTGCTGAACGCCAAGCTGATCACCCAGA |
| | | GAAAGTTCGACAACCTGACCAAGGCCGAGAGAGGCGGCCTGAGCGAGCTGGACAAGGCC |
| | | GGCTTCATCAAGAGACAGCTGGTGGAGACCAGACAGATCACCAAGCACGTGGCCCAGAT |
| | | CCTGGACAGCAGAATGAACACCAAGTACGACGAGAACGACAAGCTGATCAGAGAGGTGA |
| | | AGGTGATCACCCTGAAGAGTAAGCTGGTGAGCGACTTCAGAAAGGACTTCCAGTTCTAC |
| | | AAGGTGAGAGAGATCAACAACTACCACCACGCCCACGACGCCTACCTGAACGCCGTGGT |
| | | GGGCACCGCCCTGATCAAGAAGTACCCCAAGCTGGAGAGCGAGTTCGTGTACGGCGACT |
| | | ACAAGGTGTACGACGTGAGAAAGATGATCGCCAAGAGCGAGCAGGAGATCGGCAAGGCC |
| | | ACCGCCAAGTACTTCTTCTACAGCAACATCATGAACTTCTTCAAGACCGAGATCACCCT |
| | | GGCCAACGGCGAGATCAGAAAGAGACCCCTGATCGAGACCAACGGCGAGACCGGCGAGA |
| | | TCGTGTGGGACAAGGGCAGAGACTTCGCCACCGTGAGAAAGGTGCTGAGCATGCCCCAG |
| | | GTGAACATCGTGAAGAAGACCGAGGTGCAGACCGGCGGCTTCAGCAAGGAGAGCATCCT |
| | | GCCCAAGAGAAACAGCGACAAGCTGATCGCCAGAAAGAAGGACTGGGACCCCAAGAAGT |
| | | ACGGCGGCTTCGACAGCCCCACCGTGGCCTACAGCGTGCTGGTGGTGGCCAAGGTGGAG |
| | | AAGGGCAAGAGCAAGAAGCTGAAGAGTGTGAAGGAGCTGCTGGGCATCACCATCATGGA |
| | | GAGAAGCAGCTTCGAGAAGAACCCCATCGACTTCCTGGAGGCCAAGGGCTACAAGGAGG |
| | | TGAAGAAGGACCTGATCATCAAGCTGCCCAAGTACAGCCTGTTCGAGCTGGAGAACGGC |
| | | AGAAAGAGAATGCTGGCCAGCGCCGGCGAGCTGCAGAAGGGCAACGAGCTGGCCCTGCC |
| | | CAGCAAGTACGTGAACTTCCTGTACCTGGCCAGCCACTACGAGAAGCTGAAGGGCAGCC |
| | | CCGAGGACAACGAGCAGAAGCAGCTGTTCGTGGAGCAGCACAAGCACTACCTGGACGAG |
| | | ATCATCGAGCAGATCAGCGAGTTCAGCAAGAGAGTGATCCTGGCCGACGCCAACCTGGA |
| | | CAAGGTGCTGAGCGCCTACAACAAGCACAGAGACAAGCCCATCAGAGAGCAGGCCGAGA |
| | | ACATCATCCACCTGTTCACCCTGACCAACCTGGGCGCCCCCGCCGCCTTCAAGTACTTC |
| | | GACACCACCATCGACAGAAAGAGATACACCAGCACCAAGGAGGTGCTGGACGCCACCCT |
| | | GATCCACCAGAGCATCACCGGCCTGTACGAGACCAGAATCGACCTGAGCCAGCTGGGCG |
| | | GCGACAGCGGCGGCAGCAGCGGCGGCAGCAGCGGCAGCGAGACCCCCGGCACCAGCGAG |
| | | AGCGCCACCCCCGAGAGCAGCGGCGGCAGCAGCGGCGGCAGCAGCACCCTGAACATCGA |
| | | GGACGAGTACAGACTGCACGAGACCAGCAAGGAGCCCGACGTGAGCCTGGGCAGCACCT |
| | | GGCTGAGCGACTTCCCCCAGGCCTGGGCCGAGACCGGCGGCATGGGCCTGGCCGTGAGA |
| | | CAGGCCCCCCTGATCATCCCCCTGAAGGCCACCAGCACCCCCGTGAGCATCAAGCAGTA |
| | | CCCCATGAGCCAGGAGGCCAGACTGGGCATCAAGCCCCACATCCAGAGACTGCTGGACC |
| | | AGGGCATCCTGGTGCCCTGCCAGAGCCCCTGGAACACCCCCCTGCTGCCCGTGAAGAAG |
| | | CCCGGCACCAACGACTACAGACCCGTGCAGGACCTGAGAGAGGTGAACAAGAGAGTGGA |
| | | GGACATCCACCCCACCGTGCCCAACCCCTACAACCTGCTGAGCGGCCTGCCCCCCAGCC |
| | | ACCAGTGGTACACCGTGCTGGACCTGAAGGACGCCTTCTTCTGCCTGAGACTGCACCCC |
| | | ACCAGCCAGCCCCTGTTCGCCTTCGAGTGGAGAGACCCCGAGATGGGCATCAGCGGCCA |
| | | GCTGACCTGGACCAGACTGCCCCAGGGCTTCAAGAACAGCCCCACCCTGTTCAACGAGG |
| | | CCCTGCACAGAGACCTGGCCGACTTCAGAATCCAGCACCCCGACCTGATCCTGCTGCAG |
| | | TACGTGGACGACCTGCTGCTGGCCGCCACCAGCGAGCTGGACTGCCAGCAGGGCACCAG |
| | | AGCCCTGCTGCAGACCCTGGGCAACCTGGGCTACAGAGCCAGCGCCAAGAAGGCCCAGA |
| | | TCTGCCAGAAGCAGGTGAAGTACCTGGGCTACCTGCTGAAGGAGGGCCAGAGATGGCTG |
| | | ACCGAGGCCAGAAAGGAGACCGTGATGGGCCAGCCCACCCCCAAGACCCCCAGACAGCT |
| | | GAGAGAGTTCCTGGGCAAGGCCGGCTTCTGCAGACTGTTCATCCCCGGCTTCGCCGAGA |
| | | TGGCCGCCCCCCTGTACCCCCTGACCAAGCCCGGCACCCTGTTCAACTGGGGCCCCGAC |
| | | CAGCAGAAGGCCTACCAGGAGATCAAGCAGGCCCTGCTGACCGCCCCCGCCCTGGGCCT |
| | | GCCCGACCTGACCAAGCCCTTCGAGCTGTTCGTGGACGAGAAGCAGGGCTACGCCAAGG |
| | | GCGTGCTGACCCAGAAGCTGGGCCCCTGGAGAAGACCCGTGGCCTACCTGAGCAAGAAG |
| | | CTGGACCCCGTGGCCGCCGGCTGGCCCCCCTGCCTGAGAATGGTGGCCGCCATCGCCGT |
| | | GCTGACCAAGGACGCCGGCAAGCTGACCATGGGCCAGCCCCTGGTGATCCTGGCCCCCC |
| | | ACGCCGTGGAGGCCCTGGTGAAGCAGCCCCCCGACAGATGGCTGAGCAACGCCAGAATG |
| | | ACCCACTACCAGGCCCTGCTGCTGGACACCGACAGAGTGCAGTTCGGCCCCGTGGTGGC |
| | | CCTGAACCCCGCCACCCTGCTGCCCCTGCCCGAGGAGGGCCTGCAGCACAACTGCCTGG |
| | | ACATCCTGGCCGAGGCCCACGGCACCAGACCCGACCTGACCGACCAGCCCCTGCCCGAC |
| | | GCCGACCACACCTGGTACACCGACGGCAGCAGCCTGCTGCAGGAGGGCCAGAGAAAGGC |
| | | CGGCGCCGCCGTGACCACCGAGACCGAGGTGATCTGGGCCAAGGCCCTGCCCGCCGGCA |
| | | CCAGCGCCCAGAGAGCCGAGCTGATCGCCCTGACCCAGGCCCTGAAGATGGCCGAGGGC |
| | | AAGAAGCTGAACGTGTACACCGACAGCAGATACGCCTTCGCCACCGCCCACATCCACGG |
| | | CGAGATCTACAGAAGAAGAGGCTGGCTGACCAGCGAGGGCAAGGAGATCAAGAACAAGG |
| | | ACGAGATCCTGGCCCTGCTGAAGGCCCTGTTCCTGCCCAAGAGACTGAGCATCATCCAC |
| | | TGCCCCGGCCACCAGAAGGGCCACAGCGCCGAGGCCAGAGGCAACAGAATGGCCGACCA |
| | | GGCCGCCAGAAAGGCCGCCATCACCGAGACCCCCGACACCAGCACCCTGCTGATCGAGA |
| | | ACAGCAGCCCCAGCGGCGGCAGCAAGAGAACCGCCGACGGCAGCGAGTTCGAGCCCAAG |
| | | AAGAAGAGAAAGGTGTGA |
| 637 | PL1934 | MPAAKRVKLDGGSKDKKSDGKDSQKKGSRALWIRLSRVTDATTSPERQLESCQQLCAQR |
| | _AA | GWDVVGVAEDLDVSGAVDPFDRKRRPNLARWLAFEEQPFDVIVAYRVDRLTRSIRHLQQ |
| | | LVHWAEDHKKLVVSATEAHFDTTTPFAAVVIALMGTVAQMELEAIKERNRSAAHFNIRA |
| | | GKYRGSLPPWGYLPTRVDGEWRLVPDPVQRERILEVYHRWVDNHEPLHLVAHDLNRRGV |
| | | LSPKDYFAQLQGREPQGREWSATALKRSMISEAMLGYATLNGKTVRDDDGAPLVRAEPI |
| | | LTREQLEALRAELVKTSRAKPAVSTPSLLLRVLFCAVCGEPAYKFAGGGRKHPRYRCRS |
| | | MGFPKHCGNGTVAMAEWDAFCEEQVLDLLGDAERLEKVWVAGSDSAVELAEVNAELVDL |
| | | TSLIGSPAYRAGSPQREALDARIAALAARQEELEGLEARPSGWEWRETGQRFGDWWREQ |
| | | DTAAKNTWLRSMNVRLTFDVRGGLTRTIDFGDLQEYEQHLRLGSVVERLHTGMSGGSPK |
| | | KKRKVGSGATNFSLLKQAGDVEENPGPKRTADGSEFESPKKKRKVDKKYSIGLDIGTNS |
| | | VGWAVITDEYKVPSKKFKVLGNTDRHSIKKNLIGALLFDSGETAEATRLKRTARRRYTR |
| | | RKNRICYLQEIFSNEMAKVDDSFFHRLEESFLVEEDKKHERHPIFGNIVDEVAYHEKYP |
| | | TIYHLRKKLVDSTDKADLRLIYLALAHMIKFRGHFLIEGDLNPDNSDVDKLFIQLVQTY |
| | | NQLFEENPINASGVDAKAILSARLSKSRRLENLIAQLPGEKKNGLFGNLIALSLGLTPN |
| | | FKSNFDLAEDAKLQLSKDTYDDDLDNLLAQIGDQYADLFLAAKNLSDAILLSDILRVNT |
| | | EITKAPLSASMIKRYDEHHQDLTLLKALVRQQLPEKYKEIFFDQSKNGYAGYIDGGASQ |
| | | EEFYKFIKPILEKMDGTEELLVKLNREDLLRKQRTFDNGSIPHQIHLGELHAILRRQED |
| | | FYPFLKDNREKIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLKRRRYTGWGRLSR |
| | | KLINGIRDKQSGKTILDFLKSDGFANRNFMQLIHDDSLTFKEDIQKAQVSGQGDSLHEH |
| | | IANLAGSPAIKKGILQTVKWVDELVKVMGRHKPENIVIEMARENQTTQKGQKNSRERMK |
| | | RIEEGIKELGSQILKEHPVENTQLQNEKLYLYYLQNGRDMYVDQELDINRLSDYDVDAI |
| | | VPQSFLKDDSIDNKVLTRSDKNRGKSDNVPSEEVVKKMKNYWRQLLNAKLITQRKFDNL |
| | | TKAERGGLSELDKAGFIKRQLVETRQITKHVAQILDSRMNTKYDENDKLIREVKVITLK |
| | | SKLVSDFRKDFQFYKVREINNYHHAHDAYLNAVVGTALIKKYPKLESEFVYGDYKVYDV |
| | | RKMIAKSEQEIGKATAKYFFYSNIMNFFKTEITLANGEIRKRPLIETNGETGEIVWDKG |
| | | RDFATVRKVLSMPQVNIVKKTEVQTGGFSKESILPKRNSDKLIARKKDWDPKKYGGFDS |
| | | PTVAYSVLVVAKVEKGKSKKLKSVKELLGITIMERSSFEKNPIDFLEAKGYKEVKKDLI |
| | | IKLPKYSLFELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAENIIHLF |
| | | TLTNLGAPAAFKYFDTTIDRKRYTSTKEVLDATLIHQSITGLYETRIDLSQLGGDSGGS |
| | | SGGSSGSETPGTSESATPESSGGSSGGSSTLNIEDEYRLHETSKEPDVSLGSTWLSDFP |
| | | QAWAETGGMGLAVRQAPLIIPLKATSTPVSIKQYPMSQEARLGIKPHIQRLLDQGILVP |
| | | CQSPWNTPLLPVKKPGTNDYRPVQDLREVNKRVEDIHPTVPNPYNLLSGLPPSHQWYTV |
| | | LDLKDAFFCLRLHPTSQPLFAFEWRDPEMGISGQLTWTRLPQGFKNSPTLFNEALHRDL |
| | | ADFRIQHPDLILLQYVDDLLLAATSELDCQQGTRALLQTLGNLGYRASAKKAQICQKQV |
| | | KYLGYLLKEGQRWLTEARKETVMGQPTPKTPRQLREFLGKAGFCRLFIPGFAEMAAPLY |
| | | PLTKPGTLFNWGPDQQKAYQEIKQALLTAPALGLPDLTKPFELFVDEKQGYAKGVLTQK |
| | | LGPWRRPVAYLSKKLDPVAAGWPPCLRMVAAIAVLTKDAGKLTMGQPLVILAPHAVEAL |
| | | VKQPPDRWLSNARMTHYQALLLDTDRVQFGPVVALNPATLLPLPEEGLQHNCLDILAEA |
| | | HGTRPDLTDQPLPDADHTWYTDGSSLLQEGQRKAGAAVTTETEVIWAKALPAGTSAQRA |
| | | ELIALTQALKMAEGKKLNVYTDSRYAFATAHIHGEIYRRRGWLTSEGKEIKNKDEILAL |
| | | LKALFLPKRLSIIHCPGHQKGHSAEARGNRMADQAARKAAITETPDTSTLLIENSSPSG |
| | | GSKRTADGSEFEPKKKRKVGGSKDKKSDGKDSQKK* |
| 638 | PL1934_ | ATGcctgctgctaagagagtgaaactggatggcggcagcAAGGACAAGAAATCCGATGG |
| | DNA | CAAGGACAGCCAGAAAAAAGGCAGCAGAGCCCTGGTGGTGATCAGACTGAGCAGAGTGA |
| | | CCGACGCCACCACCAGCCCCGAGAGACAGCTGGAGAGCTGCCAGCAGCTGTGCGCCCAG |
| | | AGAGGCTGGGACGTGGTGGGCGTGGCCGAGGACCTGGACGTGAGCGGCGCCGTGGACCC |
| | | CTTCGACAGAAAGAGAAGACCCAACCTGGCCAGATGGCTGGCCTTCGAGGAGCAGCCCT |
| | | TCGACGTGATCGTGGCCTACAGAGTGGACAGACTGACCAGAAGCATCAGACACCTGCAG |
| | | CAGCTGGTGCACTGGGCCGAGGACCACAAGAAGCTGGTGGTGAGCGCCACCGAGGCCCA |
| | | CTTCGACACCACCACCCCCTTCGCCGCCGTGGTGATCGCCCTGATGGGCACCGTGGCCC |
| | | AGATGGAGCTGGAGGCCATCAAGGAGAGAAACAGAAGCGCCGCCCACTTCAACATCAGA |
| | | GCCGGCAAGTACAGAGGCAGCCTGCCCCCCTGGGGCTACCTGCCCACCAGAGTGGACGG |
| | | CGAGTGGAGACTGGTGCCCGACCCCGTGCAGAGAGAGAGAATCCTGGAGGTGTACCACA |
| | | GAGTGGTGGACAACCACGAGCCCCTGCACCTGGTGGCCCACGACCTGAACAGAAGAGGC |
| | | GTGCTGAGCCCCAAGGACTACTTCGCCCAGCTGCAGGGCAGAGAGCCCCAGGGCAGAGA |
| | | GTGGAGCGCCACCGCCCTGAAGAGAAGCATGATCAGCGAGGCCATGCTGGGCTACGCCA |
| | | CCCTGAACGGCAAGACCGTGAGAGACGACGACGGCGCCCCCCTGGTGAGAGCCGAGCCC |
| | | ATCCTGACCAGAGAGCAGCTGGAGGCCCTGAGAGCCGAGCTGGTGAAGACCAGCAGAGC |
| | | CAAGCCCGCCGTGAGCACCCCCAGCCTGCTGCTGAGAGTGCTGTTCTGCGCCGTGTGCG |
| | | GCGAGCCCGCCTACAAGTTCGCCGGCGGCGGCAGAAAGCACCCCAGATACAGATGCAGA |
| | | AGCATGGGCTTCCCCAAGCACTGCGGCAACGGCACCGTGGCCATGGCCGAGTGGGACGC |
| | | CTTCTGCGAGGAGCAGGTGCTGGACCTGCTGGGCGACGCCGAGAGACTGGAGAAGGTGT |
| | | GGGTGGCCGGCAGCGACAGCGCCGTGGAGCTGGCCGAGGTGAACGCCGAGCTGGTGGAC |
| | | CTGACCAGCCTGATCGGCAGCCCCGCCTACAGAGCCGGCAGCCCCCAGAGAGAGGCCCT |
| | | GGACGCCAGAATCGCCGCCCTGGCCGCCAGACAGGAGGAGCTGGAGGGCCTGGAGGCCA |
| | | GACCCAGCGGCTGGGAGTGGAGAGAGACCGGCCAGAGATTCGGCGACTGGTGGAGAGAG |
| | | CAGGACACCGCCGCCAAGAACACCTGGCTGAGAAGCATGAACGTGAGACTGACCTTCGA |
| | | CGTGAGAGGCGGCCTGACCAGAACCATCGACTTCGGCGACCTGCAGGAGTACGAGCAGC |
| | | ACCTGAGACTGGGCAGCGTGGTGGAGAGACTGCACACCGGCATGAGCGGCGGCAGCCCC |
| | | AAGAAGAAGAGAAAGGTGGGCAGCGGCGCCACCAACTTCAGCCTGCTGAAGCAGGCCGG |
| | | CGACGTGGAGGAGAACCCCGGCCCCAAGAGAACCGCCGACGGCAGCGAGTTCGAGAGCC |
| | | CCAAGAAGAAGAGAAAGGTGGACAAGAAGTACAGCATCGGCCTGGACATCGGCACCAAC |
| | | AGCGTGGGCTGGGCCGTGATCACCGACGAGTACAAGGTGCCCAGCAAGAAGTTCAAGGT |
| | | GCTGGGCAACACCGACAGACACAGCATCAAGAAGAACCTGATCGGCGCCCTGCTGTTCG |
| | | ACAGCGGCGAGACCGCCGAGGCCACCAGACTGAAGAGAACCGCCAGAAGAAGATACACC |
| | | AGAAGAAAGAACAGAATCTGCTACCTGCAGGAGATCTTCAGCAACGAGATGGCCAAGGT |
| | | GGACGACAGCTTCTTCCACAGACTGGAGGAGAGCTTCCTGGTGGAGGAGGACAAGAAGC |
| | | ACGAGAGACACCCCATCTTCGGCAACATCGTGGACGAGGTGGCCTACCACGAGAAGTAC |
| | | CCCACCATCTACCACCTGAGAAAGAAGCTGGTGGACAGCACCGACAAGGCCGACCTGAG |
| | | ACTGATCTACCTGGCCCTGGCCCACATGATCAAGTTCAGAGGCCACTTCCTGATCGAGG |
| | | GCGACCTGAACCCCGACAACAGCGACGTGGACAAGCTGTTCATCCAGCTGGTGCAGACC |
| | | TACAACCAGCTGTTCGAGGAGAACCCCATCAACGCCAGCGGCGTGGACGCCAAGGCCAT |
| | | CCTGAGCGCCAGACTGAGCAAGAGCAGAAGACTGGAGAACCTGATCGCCCAGCTGCCCG |
| | | GCGAGAAGAAGAACGGCCTGTTCGGCAACCTGATCGCCCTGAGCCTGGGCCTGACCCCC |
| | | AACTTCAAGAGCAACTTCGACCTGGCCGAGGACGCCAAGCTGCAGCTGAGCAAGGACAC |
| | | CTACGACGACGACCTGGACAACCTGCTGGCCCAGATCGGCGACCAGTACGCCGACCTGT |
| | | TCCTGGCCGCCAAGAACCTGAGCGACGCCATCCTGCTGAGCGACATCCTGAGAGTGAAC |
| | | ACCGAGATCACCAAGGCCCCCCTGAGCGCCAGCATGATCAAGAGATACGACGAGCACCA |
| | | CCAGGACCTGACCCTGCTGAAGGCCCTGGTGAGACAGCAGCTGCCCGAGAAGTACAAGG |
| | | AGATCTTCTTCGACCAGAGCAAGAACGGCTACGCCGGCTACATCGACGGCGGCGCCAGC |
| | | CAGGAGGAGTTCTACAAGTTCATCAAGCCCATCCTGGAGAAGATGGACGGCACCGAGGA |
| | | GCTGCTGGTGAAGCTGAACAGAGAGGACCTGCTGAGAAAGCAGAGAACCTTCGACAACG |
| | | GCAGCATCCCCCACCAGATCCACCTGGGCGAGCTGCACGCCATCCTGAGAAGACAGGAG |
| | | GACTTCTACCCCTTCCTGAAGGACAACAGAGAGAAGATCGAGAAGATCCTGACCTTCAG |
| | | AATCCCCTACTACGTGGGCCCCCTGGCCAGAGGCAACAGCAGATTCGCCTGGATGACCA |
| | | GAAAGAGCGAGGAGACCATCACCCCCTGGAACTTCGAGGAGGTGGTGGACAAGGGCGCC |
| | | AGCGCCCAGAGCTTCATCGAGAGAATGACCAACTTCGACAAGAACCTGCCCAACGAGAA |
| | | GGTGCTGCCCAAGCACAGCCTGCTGTACGAGTACTTCACCGTGTACAACGAGCTGACCA |
| | | AGGTGAAGTACGTGACCGAGGGCATGAGAAAGCCCGCCTTCCTGAGCGGCGAGCAGAAG |
| | | AAGGCCATCGTGGACCTGCTGTTCAAGACCAACAGAAAGGTGACCGTGAAGCAGCTGAA |
| | | GGAGGACTACTTCAAGAAGATCGAGTGCTTCGACAGCGTGGAGATCAGCGGCGTGGAGG |
| | | ACAGATTCAACGCCAGCCTGGGCACCTACCACGACCTGCTGAAGATCATCAAGGACAAG |
| | | GACTTCCTGGACAACGAGGAGAACGAGGACATCCTGGAGGACATCGTGCTGACCCTGAC |
| | | CCTGTTCGAGGACAGAGAGATGATCGAGGAGAGACTGAAGACCTACGCCCACCTGTTCG |
| | | ACGACAAGGTGATGAAGCAGCTGAAGAGAAGAAGATACACCGGCTGGGGCAGACTGAGC |
| | | AGAAAGCTGATCAACGGCATCAGAGACAAGCAGAGCGGCAAGACCATCCTGGACTTCCT |
| | | GAAGAGTGACGGCTTCGCCAACAGAAACTTCATGCAGCTGATCCACGACGACAGCCTGA |
| | | CCTTCAAGGAGGACATCCAGAAGGCCCAGGTGAGCGGCCAGGGCGACAGCCTGCACGAG |
| | | CACATCGCCAACCTGGCCGGCAGCCCCGCCATCAAGAAGGGCATCCTGCAGACCGTGAA |
| | | GGTGGTGGACGAGCTGGTGAAGGTGATGGGCAGACACAAGCCCGAGAACATCGTGATCG |
| | | AGATGGCCAGAGAGAACCAGACCACCCAGAAGGGCCAGAAGAACAGCAGAGAGAGAATG |
| | | AAGAGAATCGAGGAGGGCATCAAGGAGCTGGGCAGCCAGATCCTGAAGGAGCACCCCGT |
| | | GGAGAACACCCAGCTGCAGAACGAGAAGCTGTACCTGTACTACCTGCAGAACGGCAGAG |
| | | ACATGTACGTGGACCAGGAGCTGGACATCAACAGACTGAGCGACTACGACGTGGACGCC |
| | | ATCGTGCCCCAGAGCTTCCTGAAGGACGACAGCATCGACAACAAGGTGCTGACCAGAAG |
| | | CGACAAGAACAGAGGCAAGAGCGACAACGTGCCCAGCGAGGAGGTGGTGAAGAAGATGA |
| | | AGAACTACTGGAGACAGCTGCTGAACGCCAAGCTGATCACCCAGAGAAAGTTCGACAAC |
| | | CTGACCAAGGCCGAGAGAGGCGGCCTGAGCGAGCTGGACAAGGCCGGCTTCATCAAGAG |
| | | ACAGCTGGTGGAGACCAGACAGATCACCAAGCACGTGGCCCAGATCCTGGACAGCAGAA |
| | | TGAACACCAAGTACGACGAGAACGACAAGCTGATCAGAGAGGTGAAGGTGATCACCCTG |
| | | AAGAGTAAGCTGGTGAGCGACTTCAGAAAGGACTTCCAGTTCTACAAGGTGAGAGAGAT |
| | | CAACAACTACCACCACGCCCACGACGCCTACCTGAACGCCGTGGTGGGCACCGCCCTGA |
| | | TCAAGAAGTACCCCAAGCTGGAGAGCGAGTTCGTGTACGGCGACTACAAGGTGTACGAC |
| | | GTGAGAAAGATGATCGCCAAGAGCGAGCAGGAGATCGGCAAGGCCACCGCCAAGTACTT |
| | | CTTCTACAGCAACATCATGAACTTCTTCAAGACCGAGATCACCCTGGCCAACGGCGAGA |
| | | TCAGAAAGAGACCCCTGATCGAGACCAACGGCGAGACCGGCGAGATCGTGTGGGACAAG |
| | | GGCAGAGACTTCGCCACCGTGAGAAAGGTGCTGAGCATGCCCCAGGTGAACATCGTGAA |
| | | GAAGACCGAGGTGCAGACCGGCGGCTTCAGCAAGGAGAGCATCCTGCCCAAGAGAAACA |
| | | GCGACAAGCTGATCGCCAGAAAGAAGGACTGGGACCCCAAGAAGTACGGCGGCTTCGAC |
| | | AGCCCCACCGTGGCCTACAGCGTGCTGGTGGTGGCCAAGGTGGAGAAGGGCAAGAGCAA |
| | | GAAGCTGAAGAGTGTGAAGGAGCTGCTGGGCATCACCATCATGGAGAGAAGCAGCTTCG |
| | | AGAAGAACCCCATCGACTTCCTGGAGGCCAAGGGCTACAAGGAGGTGAAGAAGGACCTG |
| | | ATCATCAAGCTGCCCAAGTACAGCCTGTTCGAGCTGGAGAACGGCAGAAAGAGAATGCT |
| | | GGCCAGCGCCGGCGAGCTGCAGAAGGGCAACGAGCTGGCCCTGCCCAGCAAGTACGTGA |
| | | ACTTCCTGTACCTGGCCAGCCACTACGAGAAGCTGAAGGGCAGCCCCGAGGACAACGAG |
| | | CAGAAGCAGCTGTTCGTGGAGCAGCACAAGCACTACCTGGACGAGATCATCGAGCAGAT |
| | | CAGCGAGTTCAGCAAGAGAGTGATCCTGGCCGACGCCAACCTGGACAAGGTGCTGAGCG |
| | | CCTACAACAAGCACAGAGACAAGCCCATCAGAGAGCAGGCCGAGAACATCATCCACCTG |
| | | TTCACCCTGACCAACCTGGGCGCCCCCGCCGCCTTCAAGTACTTCGACACCACCATCGA |
| | | CAGAAAGAGATACACCAGCACCAAGGAGGTGCTGGACGCCACCCTGATCCACCAGAGCA |
| | | TCACCGGCCTGTACGAGACCAGAATCGACCTGAGCCAGCTGGGCGGCGACAGCGGCGGC |
| | | AGCAGCGGCGGCAGCAGCGGCAGCGAGACCCCCGGCACCAGCGAGAGCGCCACCCCCGA |
| | | GAGCAGCGGCGGCAGCAGCGGCGGCAGCAGCACCCTGAACATCGAGGACGAGTACAGAC |
| | | TGCACGAGACCAGCAAGGAGCCCGACGTGAGCCTGGGCAGCACCTGGCTGAGCGACTTC |
| | | CCCCAGGCCTGGGCCGAGACCGGCGGCATGGGCCTGGCCGTGAGACAGGCCCCCCTGAT |
| | | CATCCCCCTGAAGGCCACCAGCACCCCCGTGAGCATCAAGCAGTACCCCATGAGCCAGG |
| | | AGGCCAGACTGGGCATCAAGCCCCACATCCAGAGACTGCTGGACCAGGGCATCCTGGTG |
| | | CCCTGCCAGAGCCCCTGGAACACCCCCCTGCTGCCCGTGAAGAAGCCCGGCACCAACGA |
| | | CTACAGACCCGTGCAGGACCTGAGAGAGGTGAACAAGAGAGTGGAGGACATCCACCCCA |
| | | CCGTGCCCAACCCCTACAACCTGCTGAGCGGCCTGCCCCCCAGCCACCAGTGGTACACC |
| | | GTGCTGGACCTGAAGGACGCCTTCTTCTGCCTGAGACTGCACCCCACCAGCCAGCCCCT |
| | | GTTCGCCTTCGAGTGGAGAGACCCCGAGATGGGCATCAGCGGCCAGCTGACCTGGACCA |
| | | GACTGCCCCAGGGCTTCAAGAACAGCCCCACCCTGTTCAACGAGGCCCTGCACAGAGAC |
| | | CTGGCCGACTTCAGAATCCAGCACCCCGACCTGATCCTGCTGCAGTACGTGGACGACCT |
| | | GCTGCTGGCCGCCACCAGCGAGCTGGACTGCCAGCAGGGCACCAGAGCCCTGCTGCAGA |
| | | CCCTGGGCAACCTGGGCTACAGAGCCAGCGCCAAGAAGGCCCAGATCTGCCAGAAGCAG |
| | | GTGAAGTACCTGGGCTACCTGCTGAAGGAGGGCCAGAGATGGCTGACCGAGGCCAGAAA |
| | | GGAGACCGTGATGGGCCAGCCCACCCCCAAGACCCCCAGACAGCTGAGAGAGTTCCTGG |
| | | GCAAGGCCGGCTTCTGCAGACTGTTCATCCCCGGCTTCGCCGAGATGGCCGCCCCCCTG |
| | | TACCCCCTGACCAAGCCCGGCACCCTGTTCAACTGGGGCCCCGACCAGCAGAAGGCCTA |
| | | CCAGGAGATCAAGCAGGCCCTGCTGACCGCCCCCGCCCTGGGCCTGCCCGACCTGACCA |
| | | AGCCCTTCGAGCTGTTCGTGGACGAGAAGCAGGGCTACGCCAAGGGCGTGCTGACCCAG |
| | | AAGCTGGGCCCCTGGAGAAGACCCGTGGCCTACCTGAGCAAGAAGCTGGACCCCGTGGC |
| | | CGCCGGCTGGCCCCCCTGCCTGAGAATGGTGGCCGCCATCGCCGTGCTGACCAAGGACG |
| | | CCGGCAAGCTGACCATGGGCCAGCCCCTGGTGATCCTGGCCCCCCACGCCGTGGAGGCC |
| | | CTGGTGAAGCAGCCCCCCGACAGATGGCTGAGCAACGCCAGAATGACCCACTACCAGGC |
| | | CCTGCTGCTGGACACCGACAGAGTGCAGTTCGGCCCCGTGGTGGCCCTGAACCCCGCCA |
| | | CCCTGCTGCCCCTGCCCGAGGAGGGCCTGCAGCACAACTGCCTGGACATCCTGGCCGAG |
| | | GCCCACGGCACCAGACCCGACCTGACCGACCAGCCCCTGCCCGACGCCGACCACACCTG |
| | | GTACACCGACGGCAGCAGCCTGCTGCAGGAGGGCCAGAGAAAGGCCGGCGCCGCCGTGA |
| | | CCACCGAGACCGAGGTGATCTGGGCCAAGGCCCTGCCCGCCGGCACCAGCGCCCAGAGA |
| | | GCCGAGCTGATCGCCCTGACCCAGGCCCTGAAGATGGCCGAGGGCAAGAAGCTGAACGT |
| | | GTACACCGACAGCAGATACGCCTTCGCCACCGCCCACATCCACGGCGAGATCTACAGAA |
| | | GAAGAGGCTGGCTGACCAGCGAGGGCAAGGAGATCAAGAACAAGGACGAGATCCTGGCC |
| | | CTGCTGAAGGCCCTGTTCCTGCCCAAGAGACTGAGCATCATCCACTGCCCCGGCCACCA |
| | | GAAGGGCCACAGCGCCGAGGCCAGAGGCAACAGAATGGCCGACCAGGCCGCCAGAAAGG |
| | | CCGCCATCACCGAGACCCCCGACACCAGCACCCTGCTGATCGAGAACAGCAGCCCCAGC |
| | | GGCGGCAGCAAGAGAACCGCCGACGGCAGCGAGTTCGAGCCCAAGAAGAAGAGAAAGGT |
| | | GggcggcagcAAGGACAAAAAGAGCGACGGCAAGGACTCGCAAAAAAAATGA |
| 639 | PL1922 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKVGGSVSGW |
| | | RLFKKISGSGKDKKSDGKDSQKK* |
| 640 | PL1922_ | ATGAAGAGAACCGCCGACGGCAGCGAGTTCGAGAGCCCCAAGAAGAAGAGAAAGGTGGA |
| | DNA | CAAGAAGTACAGCATCGGCCTGGACATCGGCACCAACAGCGTGGGCTGGGCCGTGATCA |
| | | CCGACGAGTACAAGGTGCCCAGCAAGAAGTTCAAGGTGCTGGGCAACACCGACAGACAC |
| | | AGCATCAAGAAGAACCTGATCGGCGCCCTGCTGTTCGACAGCGGCGAGACCGCCGAGGC |
| | | CACCAGACTGAAGAGAACCGCCAGAAGAAGATACACCAGAAGAAAGAACAGAATCTGCT |
| | | ACCTGCAGGAGATCTTCAGCAACGAGATGGCCAAGGTGGACGACAGCTTCTTCCACAGA |
| | | CTGGAGGAGAGCTTCCTGGTGGAGGAGGACAAGAAGCACGAGAGACACCCCATCTTCGG |
| | | CAACATCGTGGACGAGGTGGCCTACCACGAGAAGTACCCCACCATCTACCACCTGAGAA |
| | | AGAAGCTGGTGGACAGCACCGACAAGGCCGACCTGAGACTGATCTACCTGGCCCTGGCC |
| | | CACATGATCAAGTTCAGAGGCCACTTCCTGATCGAGGGCGACCTGAACCCCGACAACAG |
| | | CGACGTGGACAAGCTGTTCATCCAGCTGGTGCAGACCTACAACCAGCTGTTCGAGGAGA |
| | | ACCCCATCAACGCCAGCGGCGTGGACGCCAAGGCCATCCTGAGCGCCAGACTGAGCAAG |
| | | AGCAGAAGACTGGAGAACCTGATCGCCCAGCTGCCCGGCGAGAAGAAGAACGGCCTGTT |
| | | CGGCAACCTGATCGCCCTGAGCCTGGGCCTGACCCCCAACTTCAAGAGCAACTTCGACC |
| | | TGGCCGAGGACGCCAAGCTGCAGCTGAGCAAGGACACCTACGACGACGACCTGGACAAC |
| | | CTGCTGGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCCCCCC |
| | | TGAGCGCCAGCATGATCAAGAGATACGACGAGCACCACCAGGACCTGACCCTGCTGAAG |
| | | GCCCTGGTGAGACAGCAGCTGCCCGAGAAGTACAAGGAGATCTTCTTCGACCAGAGCAA |
| | | GAACGGCTACGCCGGCTACATCGACGGCGGCGCCAGCCAGGAGGAGTTCTACAAGTTCA |
| | | TCAAGCCCATCCTGGAGAAGATGGACGGCACCGAGGAGCTGCTGGTGAAGCTGAACAGA |
| | | GAGGACCTGCTGAGAAAGCAGAGAACCTTCGACAACGGCAGCATCCCCCACCAGATCCA |
| | | CCTGGGCGAGCTGCACGCCATCCTGAGAAGACAGGAGGACTTCTACCCCTTCCTGAAGG |
| | | ACAACAGAGAGAAGATCGAGAAGATCCTGACCTTCAGAATCCCCTACTACGTGGGCCCC |
| | | CTGGCCAGAGGCAACAGCAGATTCGCCTGGATGACCAGAAAGAGCGAGGAGACCATCAC |
| | | CCCCTGGAACTTCGAGGAGGTGGTGGACAAGGGCGCCAGCGCCCAGAGCTTCATCGAGA |
| | | GAATGACCAACTTCGACAAGAACCTGCCCAACGAGAAGGTGCTGCCCAAGCACAGCCTG |
| | | CTGTACGAGTACTTCACCGTGTACAACGAGCTGACCAAGGTGAAGTACGTGACCGAGGG |
| | | CATGAGAAAGCCCGCCTTCCTGAGCGGCGAGCAGAAGAAGGCCATCGTGGACCTGCTGT |
| | | TCAAGACCAACAGAAAGGTGACCGTGAAGCAGCTGAAGGAGGACTACTTCAAGAAGATC |
| | | GAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGA |
| | | ACGAGGACATCCTGGAGGACATCGTGCTGACCCTGACCCTGTTCGAGGACAGAGAGATG |
| | | ATCGAGGAGAGACTGAAGACCTACGCCCACCTGTTCGACGACAAGGTGATGAAGCAGCT |
| | | GAAGAGAAGAAGATACACCGGCTGGGGCAGACTGAGCAGAAAGCTGATCAACGGCATCA |
| | | GAGACAAGCAGAGCGGCAAGACCATCCTGGACTTCCTGAAGAGTGACGGCTTCGCCAAC |
| | | AGAAACTTCATGCAGCTGATCCACGACGACAGCCTGACCTTCAAGGAGGACATCCAGAA |
| | | GGCCCAGGTGAGCGGCCAGGGCGACAGCCTGCACGAGCACATCGCCAACCTGGCCGGCA |
| | | GCCCCGCCATCAAGAAGGGCATCCTGCAGACCGTGAAGGTGGTGGACGAGCTGGTGAAG |
| | | GTGATGGGCAGACACAAGCCCGAGAACATCGTGATCGAGATGGCCAGAGAGAACCAGAC |
| | | CACCCAGAAGGGCCAGAAGAACAGCAGAGAGAGAATGAAGAGAATCGAGGAGGGCATCA |
| | | AGGAGCTGGGCAGCCAGATCCTGAAGGAGCACCCCGTGGAGAACACCCAGCTGCAGAAC |
| | | GAGAAGCTGTACCTGTACTACCTGCAGAACGGCAGAGACATGTACGTGGACCAGGAGCT |
| | | GGACATCAACAGACTGAGCGACTACGACGTGGACGCCATCGTGCCCCAGAGCTTCCTGA |
| | | AGGACGACAGCATCGACAACAAGGTGCTGACCAGAAGCGACAAGAACAGAGGCAAGAGC |
| | | GACAACGTGCCCAGCGAGGAGGTGGTGAAGAAGATGAAGAACTACTGGAGACAGCTGCT |
| | | GAACGCCAAGCTGATCACCCAGAGAAAGTTCGACAACCTGACCAAGGCCGAGAGAGGCG |
| | | GCCTGAGCGAGCTGGACAAGGCCGGCTTCATCAAGAGACAGCTGGTGGAGACCAGACAG |
| | | ATCACCAAGCACGTGGCCCAGATCCTGGACAGCAGAATGAACACCAAGTACGACGAGAA |
| | | CGACAAGCTGATCAGAGAGGTGAAGGTGATCACCCTGAAGAGTAAGCTGGTGAGCGACT |
| | | TCAGAAAGGACTTCCAGTTCTACAAGGTGAGAGAGATCAACAACTACCACCACGCCCAC |
| | | GACGCCTACCTGAACGCCGTGGTGGGCACCGCCCTGATCAAGAAGTACCCCAAGCTGGA |
| | | GAGCGAGTTCGTGTACGGCGACTACAAGGTGTACGACGTGAGAAAGATGATCGCCAAGA |
| | | GCGAGCAGGAGATCGGCAAGGCCACCGCCAAGTACTTCTTCTACAGCAACATCATGAAC |
| | | TTCTTCAAGACCGAGATCACCCTGGCCAACGGCGAGATCAGAAAGAGACCCCTGATCGA |
| | | GACCAACGGCGAGACCGGCGAGATCGTGTGGGACAAGGGCAGAGACTTCGCCACCGTGA |
| | | GAAAGGTGCTGAGCATGCCCCAGGTGAACATCGTGAAGAAGACCGAGGTGCAGACCGGC |
| | | GGCTTCAGCAAGGAGAGCATCCTGCCCAAGAGAAACAGCGACAAGCTGATCGCCAGAAA |
| | | GAAGGACTGGGACCCCAAGAAGTACGGCGGCTTCGACAGCCCCACCGTGGCCTACAGCG |
| | | TGCTGGTGGTGGCCAAGGTGGAGAAGGGCAAGAGCAAGAAGCTGAAGAGTGTGAAGGAG |
| | | CTGCTGGGCATCACCATCATGGAGAGAAGCAGCTTCGAGAAGAACCCCATCGACTTCCT |
| | | GGAGGCCAAGGGCTACAAGGAGGTGAAGAAGGACCTGATCATCAAGCTGCCCAAGTACA |
| | | GCCTGTTCGAGCTGGAGAACGGCAGAAAGAGAATGCTGGCCAGCGCCGGCGAGCTGCAG |
| | | AAGGGCAACGAGCTGGCCCTGCCCAGCAAGTACGTGAACTTCCTGTACCTGGCCAGCCA |
| | | CTACGAGAAGCTGAAGGGCAGCCCCGAGGACAACGAGCAGAAGCAGCTGTTCGTGGAGC |
| | | AGCACAAGCACTACCTGGACGAGATCATCGAGCAGATCAGCGAGTTCAGCAAGAGAGTG |
| | | ATCCTGGCCGACGCCAACCTGGACAAGGTGCTGAGCGCCTACAACAAGCACAGAGACAA |
| | | GCCCATCAGAGAGCAGGCCGAGAACATCATCCACCTGTTCACCCTGACCAACCTGGGCG |
| | | CCCCCGCCGCCTTCAAGTACTTCGACACCACCATCGACAGAAAGAGATACACCAGCACC |
| | | AAGGAGGTGCTGGACGCCACCCTGATCCACCAGAGCATCACCGGCCTGTACGAGACCAG |
| | | AATCGACCTGAGCCAGCTGGGCGGCGACAGCGGCGGCAGCAGCGGCGGCAGCAGCGGCA |
| | | GCGAGACCCCCGGCACCAGCGAGAGCGCCACCCCCGAGAGCAGCGGCGGCAGCAGCGGC |
| | | GGCAGCAGCACCCTGAACATCGAGGACGAGTACAGACTGCACGAGACCAGCAAGGAGCC |
| | | CGACGTGAGCCTGGGCAGCACCTGGCTGAGCGACTTCCCCCAGGCCTGGGCCGAGACCG |
| | | GCGGCATGGGCCTGGCCGTGAGACAGGCCCCCCTGATCATCCCCCTGAAGGCCACCAGC |
| | | ACCCCCGTGAGCATCAAGCAGTACCCCATGAGCCAGGAGGCCAGACTGGGCATCAAGCC |
| | | CCACATCCAGAGACTGCTGGACCAGGGCATCCTGGTGCCCTGCCAGAGCCCCTGGAACA |
| | | CCCCCCTGCTGCCCGTGAAGAAGCCCGGCACCAACGACTACAGACCCGTGCAGGACCTG |
| | | AGAGAGGTGAACAAGAGAGTGGAGGACATCCACCCCACCGTGCCCAACCCCTACAACCT |
| | | GCTGAGCGGCCTGCCCCCCAGCCACCAGTGGTACACCGTGCTGGACCTGAAGGACGCCT |
| | | TCTTCTGCCTGAGACTGCACCCCACCAGCCAGCCCCTGTTCGCCTTCGAGTGGAGAGAC |
| | | CCCGAGATGGGCATCAGCGGCCAGCTGACCTGGACCAGACTGCCCCAGGGCTTCAAGAA |
| | | CAGCCCCACCCTGTTCAACGAGGCCCTGCACAGAGACCTGGCCGACTTCAGAATCCAGC |
| | | ACCCCGACCTGATCCTGCTGCAGTACGTGGACGACCTGCTGCTGGCCGCCACCAGCGAG |
| | | CTGGACTGCCAGCAGGGCACCAGAGCCCTGCTGCAGACCCTGGGCAACCTGGGCTACAG |
| | | AGCCAGCGCCAAGAAGGCCCAGATCTGCCAGAAGCAGGTGAAGTACCTGGGCTACCTGC |
| | | TGAAGGAGGGCCAGAGATGGCTGACCGAGGCCAGAAAGGAGACCGTGATGGGCCAGCCC |
| | | ACCCCCAAGACCCCCAGACAGCTGAGAGAGTTCCTGGGCAAGGCCGGCTTCTGCAGACT |
| | | GTTCATCCCCGGCTTCGCCGAGATGGCCGCCCCCCTGTACCCCCTGACCAAGCCCGGCA |
| | | CCCTGTTCAACTGGGGCCCCGACCAGCAGAAGGCCTACCAGGAGATCAAGCAGGCCCTG |
| | | CTGACCGCCCCCGCCCTGGGCCTGCCCGACCTGACCAAGCCCTTCGAGCTGTTCGTGGA |
| | | CGAGAAGCAGGGCTACGCCAAGGGCGTGCTGACCCAGAAGCTGGGCCCCTGGAGAAGAC |
| | | CCGTGGCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATGGGCCA |
| | | GCCCCTGGTGATCCTGGCCCCCCACGCCGTGGAGGCCCTGGTGAAGCAGCCCCCCGACA |
| | | GATGGCTGAGCAACGCCAGAATGACCCACTACCAGGCCCTGCTGCTGGACACCGACAGA |
| | | GTGCAGTTCGGCCCCGTGGTGGCCCTGAACCCCGCCACCCTGCTGCCCCTGCCCGAGGA |
| | | GGGCCTGCAGCACAACTGCCTGGACATCCTGGCCGAGGCCCACGGCACCAGACCCGACC |
| | | TGACCGACCAGCCCCTGCCCGACGCCGACCACACCTGGTACACCGACGGCAGCAGCCTG |
| | | CTGCAGGAGGGCCAGAGAAAGGCCGGCGCCGCCGTGACCACCGAGACCGAGGTGATCTG |
| | | GGCCAAGGCCCTGCCCGCCGGCACCAGCGCCCAGAGAGCCGAGCTGATCGCCCTGACCC |
| | | AGGCCCTGAAGATGGCCGAGGGCAAGAAGCTGAACGTGTACACCGACAGCAGATACGCC |
| | | TTCGCCACCGCCCACATCCACGGCGAGATCTACAGAAGAAGAGGCTGGCTGACCAGCGA |
| | | GGGCAAGGAGATCAAGAACAAGGACGAGATCCTGGCCCTGCTGAAGGCCCTGTTCCTGC |
| | | CCAAGAGACTGAGCATCATCCACTGCCCCGGCCACCAGAAGGGCCACAGCGCCGAGGCC |
| | | AGAGGCAACAGAATGGCCGACCAGGCCGCCAGAAAGGCCGCCATCACCGAGACCCCCGA |
| | | CACCAGCACCCTGCTGATCGAGAACAGCAGCCCCAGCGGCGGCAGCAAGAGAACCGCCG |
| | | ACGGCAGCGAGTTCGAGCCCAAGAAGAAGAGAAAGGTGGGCGGCAGCGTGAGCGGCTGG |
| | | AGACTGTTCAAGAAGATCAGCGGCAGCGGCAAGGACAAGAAATCCGATGGCAAGGACAG |
| | | CCAGAAAAAATGA |
| 641 | PL1923 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLWVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKVGGSVSGW |
| | | RLFKKISGSGKDKKSDGKDSQKK* |
| 642 | PL1923_ | ATGAAGAGAACCGCCGACGGCAGCGAGTTCGAGAGCCCCAAGAAGAAGAGAAAGGTGGA |
| | DNA | CAAGAAGTACAGCATCGGCCTGGACATCGGCACCAACAGCGTGGGCTGGGCCGTGATCA |
| | | CCGACGAGTACAAGGTGCCCAGCAAGAAGTTCAAGGTGCTGGGCAACACCGACAGACAC |
| | | AGCATCAAGAAGAACCTGATCGGCGCCCTGCTGTTCGACAGCGGCGAGACCGCCGAGGC |
| | | CACCAGACTGAAGAGAACCGCCAGAAGAAGATACACCAGAAGAAAGAACAGAATCTGCT |
| | | ACCTGCAGGAGATCTTCAGCAACGAGATGGCCAAGGTGGACGACAGCTTCTTCCACAGA |
| | | CTGGAGGAGAGCTTCCTGGTGGAGGAGGACAAGAAGCACGAGAGACACCCCATCTTCGG |
| | | CAACATCGTGGACGAGGTGGCCTACCACGAGAAGTACCCCACCATCTACCACCTGAGAA |
| | | AGAAGCTGGTGGACAGCACCGACAAGGCCGACCTGAGACTGATCTACCTGGCCCTGGCC |
| | | CACATGATCAAGTTCAGAGGCCACTTCCTGATCGAGGGCGACCTGAACCCCGACAACAG |
| | | CGACGTGGACAAGCTGTTCATCCAGCTGGTGCAGACCTACAACCAGCTGTTCGAGGAGA |
| | | ACCCCATCAACGCCAGCGGCGTGGACGCCAAGGCCATCCTGAGCGCCAGACTGAGCAAG |
| | | AGCAGAAGACTGGAGAACCTGATCGCCCAGCTGCCCGGCGAGAAGAAGAACGGCCTGTT |
| | | CGGCAACCTGATCGCCCTGAGCCTGGGCCTGACCCCCAACTTCAAGAGCAACTTCGACC |
| | | TGGCCGAGGACGCCAAGCTGCAGCTGAGCAAGGACACCTACGACGACGACCTGGACAAC |
| | | CTGCTGGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCCCCCC |
| | | TGAGCGCCAGCATGATCAAGAGATACGACGAGCACCACCAGGACCTGACCCTGCTGAAG |
| | | GCCCTGGTGAGACAGCAGCTGCCCGAGAAGTACAAGGAGATCTTCTTCGACCAGAGCAA |
| | | GAACGGCTACGCCGGCTACATCGACGGCGGCGCCAGCCAGGAGGAGTTCTACAAGTTCA |
| | | TCAAGCCCATCCTGGAGAAGATGGACGGCACCGAGGAGCTGCTGGTGAAGCTGAACAGA |
| | | GAGGACCTGCTGAGAAAGCAGAGAACCTTCGACAACGGCAGCATCCCCCACCAGATCCA |
| | | CCTGGGCGAGCTGCACGCCATCCTGAGAAGACAGGAGGACTTCTACCCCTTCCTGAAGG |
| | | ACAACAGAGAGAAGATCGAGAAGATCCTGACCTTCAGAATCCCCTACTACGTGGGCCCC |
| | | CTGGCCAGAGGCAACAGCAGATTCGCCTGGATGACCAGAAAGAGCGAGGAGACCATCAC |
| | | CCCCTGGAACTTCGAGGAGGTGGTGGACAAGGGCGCCAGCGCCCAGAGCTTCATCGAGA |
| | | GAATGACCAACTTCGACAAGAACCTGCCCAACGAGAAGGTGCTGCCCAAGCACAGCCTG |
| | | CTGTACGAGTACTTCACCGTGTACAACGAGCTGACCAAGGTGAAGTACGTGACCGAGGG |
| | | CATGAGAAAGCCCGCCTTCCTGAGCGGCGAGCAGAAGAAGGCCATCGTGGACCTGCTGT |
| | | TCAAGACCAACAGAAAGGTGACCGTGAAGCAGCTGAAGGAGGACTACTTCAAGAAGATC |
| | | GAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGA |
| | | ACGAGGACATCCTGGAGGACATCGTGCTGACCCTGACCCTGTTCGAGGACAGAGAGATG |
| | | ATCGAGGAGAGACTGAAGACCTACGCCCACCTGTTCGACGACAAGGTGATGAAGCAGCT |
| | | GAAGAGAAGAAGATACACCGGCTGGGGCAGACTGAGCAGAAAGCTGATCAACGGCATCA |
| | | GAGACAAGCAGAGCGGCAAGACCATCCTGGACTTCCTGAAGAGTGACGGCTTCGCCAAC |
| | | AGAAACTTCATGCAGCTGATCCACGACGACAGCCTGACCTTCAAGGAGGACATCCAGAA |
| | | GGCCCAGGTGAGCGGCCAGGGCGACAGCCTGCACGAGCACATCGCCAACCTGGCCGGCA |
| | | GCCCCGCCATCAAGAAGGGCATCCTGCAGACCGTGAAGGTGGTGGACGAGCTGGTGAAG |
| | | GTGATGGGCAGACACAAGCCCGAGAACATCGTGATCGAGATGGCCAGAGAGAACCAGAC |
| | | CACCCAGAAGGGCCAGAAGAACAGCAGAGAGAGAATGAAGAGAATCGAGGAGGGCATCA |
| | | AGGAGCTGGGCAGCCAGATCCTGAAGGAGCACCCCGTGGAGAACACCCAGCTGCAGAAC |
| | | GAGAAGCTGTACCTGTACTACCTGCAGAACGGCAGAGACATGTACGTGGACCAGGAGCT |
| | | GGACATCAACAGACTGAGCGACTACGACGTGGACGCCATCGTGCCCCAGAGCTTCCTGA |
| | | AGGACGACAGCATCGACAACAAGGTGCTGACCAGAAGCGACAAGAACAGAGGCAAGAGC |
| | | GACAACGTGCCCAGCGAGGAGGTGGTGAAGAAGATGAAGAACTACTGGAGACAGCTGCT |
| | | GAACGCCAAGCTGATCACCCAGAGAAAGTTCGACAACCTGACCAAGGCCGAGAGAGGCG |
| | | GCCTGAGCGAGCTGGACAAGGCCGGCTTCATCAAGAGACAGCTGGTGGAGACCAGACAG |
| | | ATCACCAAGCACGTGGCCCAGATCCTGGACAGCAGAATGAACACCAAGTACGACGAGAA |
| | | CGACAAGCTGATCAGAGAGGTGAAGGTGATCACCCTGAAGAGTAAGCTGGTGAGCGACT |
| | | TCAGAAAGGACTTCCAGTTCTACAAGGTGAGAGAGATCAACAACTACCACCACGCCCAC |
| | | GACGCCTACCTGAACGCCGTGGTGGGCACCGCCCTGATCAAGAAGTACCCCAAGCTGGA |
| | | GAGCGAGTTCGTGTACGGCGACTACAAGGTGTACGACGTGAGAAAGATGATCGCCAAGA |
| | | GCGAGCAGGAGATCGGCAAGGCCACCGCCAAGTACTTCTTCTACAGCAACATCATGAAC |
| | | TTCTTCAAGACCGAGATCACCCTGGCCAACGGCGAGATCAGAAAGAGACCCCTGATCGA |
| | | GACCAACGGCGAGACCGGCGAGATCGTGTGGGACAAGGGCAGAGACTTCGCCACCGTGA |
| | | GAAAGGTGCTGAGCATGCCCCAGGTGAACATCGTGAAGAAGACCGAGGTGCAGACCGGC |
| | | GGCTTCAGCAAGGAGAGCATCCTGCCCAAGAGAAACAGCGACAAGCTGATCGCCAGAAA |
| | | GAAGGACTGGGACCCCAAGAAGTACGGCGGCTTCGACAGCCCCACCGTGGCCTACAGCG |
| | | TGCTGGTGGTGGCCAAGGTGGAGAAGGGCAAGAGCAAGAAGCTGAAGAGTGTGAAGGAG |
| | | CTGCTGGGCATCACCATCATGGAGAGAAGCAGCTTCGAGAAGAACCCCATCGACTTCCT |
| | | GGAGGCCAAGGGCTACAAGGAGGTGAAGAAGGACCTGATCATCAAGCTGCCCAAGTACA |
| | | GCCTGTTCGAGCTGGAGAACGGCAGAAAGAGAATGCTGGCCAGCGCCGGCGAGCTGCAG |
| | | AAGGGCAACGAGCTGGCCCTGCCCAGCAAGTACGTGAACTTCCTGTACCTGGCCAGCCA |
| | | CTACGAGAAGCTGAAGGGCAGCCCCGAGGACAACGAGCAGAAGCAGCTGTTCGTGGAGC |
| | | AGCACAAGCACTACCTGGACGAGATCATCGAGCAGATCAGCGAGTTCAGCAAGAGAGTG |
| | | ATCCTGGCCGACGCCAACCTGGACAAGGTGCTGAGCGCCTACAACAAGCACAGAGACAA |
| | | GCCCATCAGAGAGCAGGCCGAGAACATCATCCACCTGTTCACCCTGACCAACCTGGGCG |
| | | CCCCCGCCGCCTTCAAGTACTTCGACACCACCATCGACAGAAAGAGATACACCAGCACC |
| | | AAGGAGGTGCTGGACGCCACCCTGATCCACCAGAGCATCACCGGCCTGTACGAGACCAG |
| | | AATCGACCTGAGCCAGCTGGGGGGCGACAGCGGCGGCAGCAGCGGCGGCAGCAGCGGCA |
| | | GCGAGACCCCCGGCACCAGCGAGAGCGCCACCCCCGAGAGCAGCGGCGGCAGCAGCGGC |
| | | GGCAGCAGCACCCTGAACATCGAGGACGAGTACAGACTGCACGAGACCAGCAAGGAGCC |
| | | CGACGTGAGCCTGGGCAGCACCTGGCTGAGCGACTTCCCCCAGGCCTGGGCCGAGACCG |
| | | GCGGCATGGGCCTGGCCGTGAGACAGGCCCCCCTGATCATCCCCCTGAAGGCCACCAGC |
| | | ACCCCCGTGAGCATCAAGCAGTACCCCATGAGCCAGGAGGCCAGACTGGGCATCAAGCC |
| | | CCACATCCAGAGACTGCTGGACCAGGGCATCCTGGTGCCCTGCCAGAGCCCCTGGAACA |
| | | CCCCCCTGCTGCCCGTGAAGAAGCCCGGCACCAACGACTACAGACCCGTGCAGGACCTG |
| | | AGAGAGGTGAACAAGAGAGTGGAGGACATCCACCCCACCGTGCCCAACCCCTACAACCT |
| | | GCTGAGCGGCCTGCCCCCCAGCCACCAGTGGTACACCGTGCTGGACCTGAAGGACGCCT |
| | | TCTTCTGCCTGAGACTGCACCCCACCAGCCAGCCCCTGTTCGCCTTCGAGTGGAGAGAC |
| | | CCCGAGATGGGCATCAGCGGCCAGCTGACCTGGACCAGACTGCCCCAGGGCTTCAAGAA |
| | | CAGCCCCACCCTGTTCAACGAGGCCCTGCACAGAGACCTGGCCGACTTCAGAATCCAGC |
| | | ACCCCGACCTGATCCTGCTGCAGTACGTGGACGACCTGCTGCTGGCCGCCACCAGCGAG |
| | | CTGGACTGCCAGCAGGGCACCAGAGCCCTGCTGCAGACCCTGGGCAACCTGGGCTACAG |
| | | AGCCAGCGCCAAGAAGGCCCAGATCTGCCAGAAGCAGGTGAAGTACCTGGGCTACCTGC |
| | | TGAAGGAGGGCCAGAGATGGCTGACCGAGGCCAGAAAGGAGACCGTGATGGGCCAGCCC |
| | | ACCCCCAAGACCCCCAGACAGCTGAGAGAGTTCCTGGGCAAGGCCGGCTTCTGCAGACT |
| | | GTTCATCCCCGGCTTCGCCGAGATGGCCGCCCCCCTGTACCCCCTGACCAAGCCCGGCA |
| | | CCCTGTTCAACTGGGGCCCCGACCAGCAGAAGGCCTACCAGGAGATCAAGCAGGCCCTG |
| | | CTGACCGCCCCCGCCCTGGGCCTGCCCGACCTGACCAAGCCCTTCGAGCTGTTCGTGGA |
| | | CGAGAAGCAGGGCTACGCCAAGGGCGTGCTGACCCAGAAGCTGGGCCCCTGGAGAAGAC |
| | | CCGTGGCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATGGGCCA |
| | | GCCCCTGGTGATCCTGGCCCCCCACGCCGTGGAGGCCCTGGTGAAGCAGCCCCCCGACA |
| | | GATGGCTGAGCAACGCCAGAATGACCCACTACCAGGCCCTGCTGCTGGACACCGACAGA |
| | | GTGCAGTTCGGCCCCGTGGTGGCCCTGAACCCCGCCACCCTGCTGCCCCTGCCCGAGGA |
| | | GGGCCTGCAGCACAACTGCCTGGACATCCTGGCCGAGGCCCACGGCACCAGACCCGACC |
| | | TGACCGACCAGCCCCTGCCCGACGCCGACCACACCTGGTACACCGACGGCAGCAGCCTG |
| | | CTGCAGGAGGGCCAGAGAAAGGCCGGCGCCGCCGTGACCACCGAGACCGAGGTGATCTG |
| | | GGCCAAGGCCCTGCCCGCCGGCACCAGCGCCCAGAGAGCCGAGCTGATCGCCCTGACCC |
| | | AGGCCCTGAAGATGGCCGAGGGCAAGAAGCTGAACGTGTACACCGACAGCAGATACGCC |
| | | TTCGCCACCGCCCACATCCACGGCGAGATCTACAGAAGAAGAGGCTGGCTGACCAGCGA |
| | | GGGCAAGGAGATCAAGAACAAGGACGAGATCCTGGCCCTGCTGAAGGCCCTGTTCCTGC |
| | | CCAAGAGACTGAGCATCATCCACTGCCCCGGCCACCAGAAGGGCCACAGCGCCGAGGCC |
| | | AGAGGCAACAGAATGGCCGACCAGGCCGCCAGAAAGGCCGCCATCACCGAGACCCCCGA |
| | | CACCAGCACCCTGCTGATCGAGAACAGCAGCCCCAGCGGCGGCAGCAAGAGAACCGCCG |
| | | ACGGCAGCGAGTTCGAGCCCAAGAAGAAGAGAAAGGTGGGCGGCAGCGTGAGCGGCTGG |
| | | AGACTGTTCAAGAAGATCAGCGGCAGCGGCAAGGACAAGAAATCCGATGGCAAGGACAG |
| | | CCAGAAAAAATGA |
| 643 | PL1924 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLWVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKVGSGKDKK |
| | | SDGKDSQKK* |
| 644 | PL1924_ | ATGAAGAGAACCGCCGACGGCAGCGAGTTCGAGAGCCCCAAGAAGAAGAGAAAGGTGGA |
| | DNA | CAAGAAGTACAGCATCGGCCTGGACATCGGCACCAACAGCGTGGGCTGGGCCGTGATCA |
| | | CCGACGAGTACAAGGTGCCCAGCAAGAAGTTCAAGGTGCTGGGCAACACCGACAGACAC |
| | | AGCATCAAGAAGAACCTGATCGGCGCCCTGCTGTTCGACAGCGGCGAGACCGCCGAGGC |
| | | CACCAGACTGAAGAGAACCGCCAGAAGAAGATACACCAGAAGAAAGAACAGAATCTGCT |
| | | ACCTGCAGGAGATCTTCAGCAACGAGATGGCCAAGGTGGACGACAGCTTCTTCCACAGA |
| | | CTGGAGGAGAGCTTCCTGGTGGAGGAGGACAAGAAGCACGAGAGACACCCCATCTTCGG |
| | | CAACATCGTGGACGAGGTGGCCTACCACGAGAAGTACCCCACCATCTACCACCTGAGAA |
| | | AGAAGCTGGTGGACAGCACCGACAAGGCCGACCTGAGACTGATCTACCTGGCCCTGGCC |
| | | CACATGATCAAGTTCAGAGGCCACTTCCTGATCGAGGGCGACCTGAACCCCGACAACAG |
| | | CGACGTGGACAAGCTGTTCATCCAGCTGGTGCAGACCTACAACCAGCTGTTCGAGGAGA |
| | | ACCCCATCAACGCCAGCGGCGTGGACGCCAAGGCCATCCTGAGCGCCAGACTGAGCAAG |
| | | AGCAGAAGACTGGAGAACCTGATCGCCCAGCTGCCCGGCGAGAAGAAGAACGGCCTGTT |
| | | CGGCAACCTGATCGCCCTGAGCCTGGGCCTGACCCCCAACTTCAAGAGCAACTTCGACC |
| | | TGGCCGAGGACGCCAAGCTGCAGCTGAGCAAGGACACCTACGACGACGACCTGGACAAC |
| | | CTGCTGGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCCCCCC |
| | | TGAGCGCCAGCATGATCAAGAGATACGACGAGCACCACCAGGACCTGACCCTGCTGAAG |
| | | GCCCTGGTGAGACAGCAGCTGCCCGAGAAGTACAAGGAGATCTTCTTCGACCAGAGCAA |
| | | GAACGGCTACGCCGGCTACATCGACGGCGGCGCCAGCCAGGAGGAGTTCTACAAGTTCA |
| | | TCAAGCCCATCCTGGAGAAGATGGACGGCACCGAGGAGCTGCTGGTGAAGCTGAACAGA |
| | | GAGGACCTGCTGAGAAAGCAGAGAACCTTCGACAACGGCAGCATCCCCCACCAGATCCA |
| | | CCTGGGCGAGCTGCACGCCATCCTGAGAAGACAGGAGGACTTCTACCCCTTCCTGAAGG |
| | | ACAACAGAGAGAAGATCGAGAAGATCCTGACCTTCAGAATCCCCTACTACGTGGGCCCC |
| | | CTGGCCAGAGGCAACAGCAGATTCGCCTGGATGACCAGAAAGAGCGAGGAGACCATCAC |
| | | CCCCTGGAACTTCGAGGAGGTGGTGGACAAGGGCGCCAGCGCCCAGAGCTTCATCGAGA |
| | | GAATGACCAACTTCGACAAGAACCTGCCCAACGAGAAGGTGCTGCCCAAGCACAGCCTG |
| | | CTGTACGAGTACTTCACCGTGTACAACGAGCTGACCAAGGTGAAGTACGTGACCGAGGG |
| | | CATGAGAAAGCCCGCCTTCCTGAGCGGCGAGCAGAAGAAGGCCATCGTGGACCTGCTGT |
| | | TCAAGACCAACAGAAAGGTGACCGTGAAGCAGCTGAAGGAGGACTACTTCAAGAAGATC |
| | | GAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGA |
| | | ACGAGGACATCCTGGAGGACATCGTGCTGACCCTGACCCTGTTCGAGGACAGAGAGATG |
| | | ATCGAGGAGAGACTGAAGACCTACGCCCACCTGTTCGACGACAAGGTGATGAAGCAGCT |
| | | GAAGAGAAGAAGATACACCGGCTGGGGCAGACTGAGCAGAAAGCTGATCAACGGCATCA |
| | | GAGACAAGCAGAGCGGCAAGACCATCCTGGACTTCCTGAAGAGTGACGGCTTCGCCAAC |
| | | AGAAACTTCATGCAGCTGATCCACGACGACAGCCTGACCTTCAAGGAGGACATCCAGAA |
| | | GGCCCAGGTGAGCGGCCAGGGCGACAGCCTGCACGAGCACATCGCCAACCTGGCCGGCA |
| | | GCCCCGCCATCAAGAAGGGCATCCTGCAGACCGTGAAGGTGGTGGACGAGCTGGTGAAG |
| | | GTGATGGGCAGACACAAGCCCGAGAACATCGTGATCGAGATGGCCAGAGAGAACCAGAC |
| | | CACCCAGAAGGGCCAGAAGAACAGCAGAGAGAGAATGAAGAGAATCGAGGAGGGCATCA |
| | | AGGAGCTGGGCAGCCAGATCCTGAAGGAGCACCCCGTGGAGAACACCCAGCTGCAGAAC |
| | | GAGAAGCTGTACCTGTACTACCTGCAGAACGGCAGAGACATGTACGTGGACCAGGAGCT |
| | | GGACATCAACAGACTGAGCGACTACGACGTGGACGCCATCGTGCCCCAGAGCTTCCTGA |
| | | AGGACGACAGCATCGACAACAAGGTGCTGACCAGAAGCGACAAGAACAGAGGCAAGAGC |
| | | GACAACGTGCCCAGCGAGGAGGTGGTGAAGAAGATGAAGAACTACTGGAGACAGCTGCT |
| | | GAACGCCAAGCTGATCACCCAGAGAAAGTTCGACAACCTGACCAAGGCCGAGAGAGGCG |
| | | GCCTGAGCGAGCTGGACAAGGCCGGCTTCATCAAGAGACAGCTGGTGGAGACCAGACAG |
| | | ATCACCAAGCACGTGGCCCAGATCCTGGACAGCAGAATGAACACCAAGTACGACGAGAA |
| | | CGACAAGCTGATCAGAGAGGTGAAGGTGATCACCCTGAAGAGTAAGCTGGTGAGCGACT |
| | | TCAGAAAGGACTTCCAGTTCTACAAGGTGAGAGAGATCAACAACTACCACCACGCCCAC |
| | | GACGCCTACCTGAACGCCGTGGTGGGCACCGCCCTGATCAAGAAGTACCCCAAGCTGGA |
| | | GAGCGAGTTCGTGTACGGCGACTACAAGGTGTACGACGTGAGAAAGATGATCGCCAAGA |
| | | GCGAGCAGGAGATCGGCAAGGCCACCGCCAAGTACTTCTTCTACAGCAACATCATGAAC |
| | | TTCTTCAAGACCGAGATCACCCTGGCCAACGGCGAGATCAGAAAGAGACCCCTGATCGA |
| | | GACCAACGGCGAGACCGGCGAGATCGTGTGGGACAAGGGCAGAGACTTCGCCACCGTGA |
| | | GAAAGGTGCTGAGCATGCCCCAGGTGAACATCGTGAAGAAGACCGAGGTGCAGACCGGC |
| | | GGCTTCAGCAAGGAGAGCATCCTGCCCAAGAGAAACAGCGACAAGCTGATCGCCAGAAA |
| | | GAAGGACTGGGACCCCAAGAAGTACGGCGGCTTCGACAGCCCCACCGTGGCCTACAGCG |
| | | TGCTGGTGGTGGCCAAGGTGGAGAAGGGCAAGAGCAAGAAGCTGAAGAGTGTGAAGGAG |
| | | CTGCTGGGCATCACCATCATGGAGAGAAGCAGCTTCGAGAAGAACCCCATCGACTTCCT |
| | | GGAGGCCAAGGGCTACAAGGAGGTGAAGAAGGACCTGATCATCAAGCTGCCCAAGTACA |
| | | GCCTGTTCGAGCTGGAGAACGGCAGAAAGAGAATGCTGGCCAGCGCCGGCGAGCTGCAG |
| | | AAGGGCAACGAGCTGGCCCTGCCCAGCAAGTACGTGAACTTCCTGTACCTGGCCAGCCA |
| | | CTACGAGAAGCTGAAGGGCAGCCCCGAGGACAACGAGCAGAAGCAGCTGTTCGTGGAGC |
| | | AGCACAAGCACTACCTGGACGAGATCATCGAGCAGATCAGCGAGTTCAGCAAGAGAGTG |
| | | ATCCTGGCCGACGCCAACCTGGACAAGGTGCTGAGCGCCTACAACAAGCACAGAGACAA |
| | | GCCCATCAGAGAGCAGGCCGAGAACATCATCCACCTGTTCACCCTGACCAACCTGGGCG |
| | | CCCCCGCCGCCTTCAAGTACTTCGACACCACCATCGACAGAAAGAGATACACCAGCACC |
| | | AAGGAGGTGCTGGACGCCACCCTGATCCACCAGAGCATCACCGGCCTGTACGAGACCAG |
| | | AATCGACCTGAGCCAGCTGGGGGGCGACAGCGGCGGCAGCAGCGGCGGCAGCAGCGGCA |
| | | GCGAGACCCCCGGCACCAGCGAGAGCGCCACCCCCGAGAGCAGCGGCGGCAGCAGCGGC |
| | | GGCAGCAGCACCCTGAACATCGAGGACGAGTACAGACTGCACGAGACCAGCAAGGAGCC |
| | | CGACGTGAGCCTGGGCAGCACCTGGCTGAGCGACTTCCCCCAGGCCTGGGCCGAGACCG |
| | | GCGGCATGGGCCTGGCCGTGAGACAGGCCCCCCTGATCATCCCCCTGAAGGCCACCAGC |
| | | ACCCCCGTGAGCATCAAGCAGTACCCCATGAGCCAGGAGGCCAGACTGGGCATCAAGCC |
| | | CCACATCCAGAGACTGCTGGACCAGGGCATCCTGGTGCCCTGCCAGAGCCCCTGGAACA |
| | | CCCCCCTGCTGCCCGTGAAGAAGCCCGGCACCAACGACTACAGACCCGTGCAGGACCTG |
| | | AGAGAGGTGAACAAGAGAGTGGAGGACATCCACCCCACCGTGCCCAACCCCTACAACCT |
| | | GCTGAGCGGCCTGCCCCCCAGCCACCAGTGGTACACCGTGCTGGACCTGAAGGACGCCT |
| | | TCTTCTGCCTGAGACTGCACCCCACCAGCCAGCCCCTGTTCGCCTTCGAGTGGAGAGAC |
| | | CCCGAGATGGGCATCAGCGGCCAGCTGACCTGGACCAGACTGCCCCAGGGCTTCAAGAA |
| | | CAGCCCCACCCTGTTCAACGAGGCCCTGCACAGAGACCTGGCCGACTTCAGAATCCAGC |
| | | ACCCCGACCTGATCCTGCTGCAGTACGTGGACGACCTGCTGCTGGCCGCCACCAGCGAG |
| | | CTGGACTGCCAGCAGGGCACCAGAGCCCTGCTGCAGACCCTGGGCAACCTGGGCTACAG |
| | | AGCCAGCGCCAAGAAGGCCCAGATCTGCCAGAAGCAGGTGAAGTACCTGGGCTACCTGC |
| | | TGAAGGAGGGCCAGAGATGGCTGACCGAGGCCAGAAAGGAGACCGTGATGGGCCAGCCC |
| | | ACCCCCAAGACCCCCAGACAGCTGAGAGAGTTCCTGGGCAAGGCCGGCTTCTGCAGACT |
| | | GTTCATCCCCGGCTTCGCCGAGATGGCCGCCCCCCTGTACCCCCTGACCAAGCCCGGCA |
| | | CCCTGTTCAACTGGGGCCCCGACCAGCAGAAGGCCTACCAGGAGATCAAGCAGGCCCTG |
| | | CTGACCGCCCCCGCCCTGGGCCTGCCCGACCTGACCAAGCCCTTCGAGCTGTTCGTGGA |
| | | CGAGAAGCAGGGCTACGCCAAGGGCGTGCTGACCCAGAAGCTGGGCCCCTGGAGAAGAC |
| | | CCGTGGCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATGGGCCA |
| | | GCCCCTGGTGATCCTGGCCCCCCACGCCGTGGAGGCCCTGGTGAAGCAGCCCCCCGACA |
| | | GATGGCTGAGCAACGCCAGAATGACCCACTACCAGGCCCTGCTGCTGGACACCGACAGA |
| | | GTGCAGTTCGGCCCCGTGGTGGCCCTGAACCCCGCCACCCTGCTGCCCCTGCCCGAGGA |
| | | GGGCCTGCAGCACAACTGCCTGGACATCCTGGCCGAGGCCCACGGCACCAGACCCGACC |
| | | TGACCGACCAGCCCCTGCCCGACGCCGACCACACCTGGTACACCGACGGCAGCAGCCTG |
| | | CTGCAGGAGGGCCAGAGAAAGGCCGGCGCCGCCGTGACCACCGAGACCGAGGTGATCTG |
| | | GGCCAAGGCCCTGCCCGCCGGCACCAGCGCCCAGAGAGCCGAGCTGATCGCCCTGACCC |
| | | AGGCCCTGAAGATGGCCGAGGGCAAGAAGCTGAACGTGTACACCGACAGCAGATACGCC |
| | | TTCGCCACCGCCCACATCCACGGCGAGATCTACAGAAGAAGAGGCTGGCTGACCAGCGA |
| | | GGGCAAGGAGATCAAGAACAAGGACGAGATCCTGGCCCTGCTGAAGGCCCTGTTCCTGC |
| | | CCAAGAGACTGAGCATCATCCACTGCCCCGGCCACCAGAAGGGCCACAGCGCCGAGGCC |
| | | AGAGGCAACAGAATGGCCGACCAGGCCGCCAGAAAGGCCGCCATCACCGAGACCCCCGA |
| | | CACCAGCACCCTGCTGATCGAGAACAGCAGCCCCAGCGGCGGCAGCAAGAGAACCGCCG |
| | | ACGGCAGCGAGTTCGAGCCCAAGAAGAAGAGAAAGGTGGGCAGCGGCAAGGACAAGAAA |
| | | TCCGATGGCAAGGACAGCCAGAAAAAATGA |
| 645 | PL1925 | MKRTADGSEFESPKKKRKVDKKYSIGLDIGTNSVGWAVITDEYKVPSKKFKVLGNTDRH |
| | _AA | SIKKNLIGALLFDSGETAEATRLKRTARRRYTRRKNRICYLQEIFSNEMAKVDDSFFHR |
| | | LEESFLVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTFRIPYYVGP |
| | | LARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGILQTVKVVDELVK |
| | | VMGRHKPENIVIEMARENQTTQKGQKNSRERMKRIEEGIKELGSQILKEHPVENTQLQN |
| | | EKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKTEVQTG |
| | | GFSKESILPKRNSDKLIARKKDWDPKKYGGFDSPTVAYSVLWVAKVEKGKSKKLKSVKE |
| | | LLGITIMERSSFEKNPIDFLEAKGYKEVKKDLIIKLPKYSLFELENGRKRMLASAGELQ |
| | | KGNELALPSKYVNFLYLASHYEKLKGSPEDNEQKQLFVEQHKHYLDEIIEQISEFSKRV |
| | | ILADANLDKVLSAYNKHRDKPIREQAENIIHLFTLTNLGAPAAFKYFDTTIDRKRYTST |
| | | KEVLDATLIHQSITGLYETRIDLSQLGGDSGGSSGGSSGSETPGTSESATPESSGGSSG |
| | | GSSTLNIEDEYRLHETSKEPDVSLGSTWLSDFPQAWAETGGMGLAVRQAPLIIPLKATS |
| | | TPVSIKQYPMSQEARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHPTSQPLFAFEWRD |
| | | PEMGISGQLTWTRLPQGFKNSPTLFNEALHRDLADFRIQHPDLILLQYVDDLLLAATSE |
| | | LDCQQGTRALLQTLGNLGYRASAKKAQICQKQVKYLGYLLKEGQRWLTEARKETVMGQP |
| | | TPKTPRQLREFLGKAGFCRLFIPGFAEMAAPLYPLTKPGTLFNWGPDQQKAYQEIKQAL |
| | | LTAPALGLPDLTKPFELFVDEKQGYAKGVLTQKLGPWRRPVAYLSKKLDPVAAGWPPCL |
| | | RMVAAIAVLTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTWYTDGSSL |
| | | LQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEA |
| | | RGNRMADQAARKAAITETPDTSTLLIENSSPSGGSKRTADGSEFEPKKKRKVGSGKDKK |
| | | SDGKDSQKK* |
| 646 | PL1925_ | ATGAAGAGAACCGCCGACGGCAGCGAGTTCGAGAGCCCCAAGAAGAAGAGAAAGGTGGA |
| | DNA | CAAGAAGTACAGCATCGGCCTGGACATCGGCACCAACAGCGTGGGCTGGGCCGTGATCA |
| | | CCGACGAGTACAAGGTGCCCAGCAAGAAGTTCAAGGTGCTGGGCAACACCGACAGACAC |
| | | AGCATCAAGAAGAACCTGATCGGCGCCCTGCTGTTCGACAGCGGCGAGACCGCCGAGGC |
| | | CACCAGACTGAAGAGAACCGCCAGAAGAAGATACACCAGAAGAAAGAACAGAATCTGCT |
| | | ACCTGCAGGAGATCTTCAGCAACGAGATGGCCAAGGTGGACGACAGCTTCTTCCACAGA |
| | | CTGGAGGAGAGCTTCCTGGTGGAGGAGGACAAGAAGCACGAGAGACACCCCATCTTCGG |
| | | CAACATCGTGGACGAGGTGGCCTACCACGAGAAGTACCCCACCATCTACCACCTGAGAA |
| | | AGAAGCTGGTGGACAGCACCGACAAGGCCGACCTGAGACTGATCTACCTGGCCCTGGCC |
| | | CACATGATCAAGTTCAGAGGCCACTTCCTGATCGAGGGCGACCTGAACCCCGACAACAG |
| | | CGACGTGGACAAGCTGTTCATCCAGCTGGTGCAGACCTACAACCAGCTGTTCGAGGAGA |
| | | ACCCCATCAACGCCAGCGGCGTGGACGCCAAGGCCATCCTGAGCGCCAGACTGAGCAAG |
| | | AGCAGAAGACTGGAGAACCTGATCGCCCAGCTGCCCGGCGAGAAGAAGAACGGCCTGTT |
| | | CGGCAACCTGATCGCCCTGAGCCTGGGCCTGACCCCCAACTTCAAGAGCAACTTCGACC |
| | | TGGCCGAGGACGCCAAGCTGCAGCTGAGCAAGGACACCTACGACGACGACCTGGACAAC |
| | | CTGCTGGCCCAGATCGGCGACCAGTACGCCGACCTGTTCCTGGCCGCCAAGAACCTGAG |
| | | CGACGCCATCCTGCTGAGCGACATCCTGAGAGTGAACACCGAGATCACCAAGGCCCCCC |
| | | TGAGCGCCAGCATGATCAAGAGATACGACGAGCACCACCAGGACCTGACCCTGCTGAAG |
| | | GCCCTGGTGAGACAGCAGCTGCCCGAGAAGTACAAGGAGATCTTCTTCGACCAGAGCAA |
| | | GAACGGCTACGCCGGCTACATCGACGGCGGCGCCAGCCAGGAGGAGTTCTACAAGTTCA |
| | | TCAAGCCCATCCTGGAGAAGATGGACGGCACCGAGGAGCTGCTGGTGAAGCTGAACAGA |
| | | GAGGACCTGCTGAGAAAGCAGAGAACCTTCGACAACGGCAGCATCCCCCACCAGATCCA |
| | | CCTGGGCGAGCTGCACGCCATCCTGAGAAGACAGGAGGACTTCTACCCCTTCCTGAAGG |
| | | ACAACAGAGAGAAGATCGAGAAGATCCTGACCTTCAGAATCCCCTACTACGTGGGCCCC |
| | | CTGGCCAGAGGCAACAGCAGATTCGCCTGGATGACCAGAAAGAGCGAGGAGACCATCAC |
| | | CCCCTGGAACTTCGAGGAGGTGGTGGACAAGGGCGCCAGCGCCCAGAGCTTCATCGAGA |
| | | GAATGACCAACTTCGACAAGAACCTGCCCAACGAGAAGGTGCTGCCCAAGCACAGCCTG |
| | | CTGTACGAGTACTTCACCGTGTACAACGAGCTGACCAAGGTGAAGTACGTGACCGAGGG |
| | | CATGAGAAAGCCCGCCTTCCTGAGCGGCGAGCAGAAGAAGGCCATCGTGGACCTGCTGT |
| | | TCAAGACCAACAGAAAGGTGACCGTGAAGCAGCTGAAGGAGGACTACTTCAAGAAGATC |
| | | GAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGA |
| | | ACGAGGACATCCTGGAGGACATCGTGCTGACCCTGACCCTGTTCGAGGACAGAGAGATG |
| | | ATCGAGGAGAGACTGAAGACCTACGCCCACCTGTTCGACGACAAGGTGATGAAGCAGCT |
| | | GAAGAGAAGAAGATACACCGGCTGGGGCAGACTGAGCAGAAAGCTGATCAACGGCATCA |
| | | GAGACAAGCAGAGCGGCAAGACCATCCTGGACTTCCTGAAGAGTGACGGCTTCGCCAAC |
| | | AGAAACTTCATGCAGCTGATCCACGACGACAGCCTGACCTTCAAGGAGGACATCCAGAA |
| | | GGCCCAGGTGAGCGGCCAGGGCGACAGCCTGCACGAGCACATCGCCAACCTGGCCGGCA |
| | | GCCCCGCCATCAAGAAGGGCATCCTGCAGACCGTGAAGGTGGTGGACGAGCTGGTGAAG |
| | | GTGATGGGCAGACACAAGCCCGAGAACATCGTGATCGAGATGGCCAGAGAGAACCAGAC |
| | | CACCCAGAAGGGCCAGAAGAACAGCAGAGAGAGAATGAAGAGAATCGAGGAGGGCATCA |
| | | AGGAGCTGGGCAGCCAGATCCTGAAGGAGCACCCCGTGGAGAACACCCAGCTGCAGAAC |
| | | GAGAAGCTGTACCTGTACTACCTGCAGAACGGCAGAGACATGTACGTGGACCAGGAGCT |
| | | GGACATCAACAGACTGAGCGACTACGACGTGGACGCCATCGTGCCCCAGAGCTTCCTGA |
| | | AGGACGACAGCATCGACAACAAGGTGCTGACCAGAAGCGACAAGAACAGAGGCAAGAGC |
| | | GACAACGTGCCCAGCGAGGAGGTGGTGAAGAAGATGAAGAACTACTGGAGACAGCTGCT |
| | | GAACGCCAAGCTGATCACCCAGAGAAAGTTCGACAACCTGACCAAGGCCGAGAGAGGCG |
| | | GCCTGAGCGAGCTGGACAAGGCCGGCTTCATCAAGAGACAGCTGGTGGAGACCAGACAG |
| | | ATCACCAAGCACGTGGCCCAGATCCTGGACAGCAGAATGAACACCAAGTACGACGAGAA |
| | | CGACAAGCTGATCAGAGAGGTGAAGGTGATCACCCTGAAGAGTAAGCTGGTGAGCGACT |
| | | TCAGAAAGGACTTCCAGTTCTACAAGGTGAGAGAGATCAACAACTACCACCACGCCCAC |
| | | GACGCCTACCTGAACGCCGTGGTGGGCACCGCCCTGATCAAGAAGTACCCCAAGCTGGA |
| | | GAGCGAGTTCGTGTACGGCGACTACAAGGTGTACGACGTGAGAAAGATGATCGCCAAGA |
| | | GCGAGCAGGAGATCGGCAAGGCCACCGCCAAGTACTTCTTCTACAGCAACATCATGAAC |
| | | TTCTTCAAGACCGAGATCACCCTGGCCAACGGCGAGATCAGAAAGAGACCCCTGATCGA |
| | | GACCAACGGCGAGACCGGCGAGATCGTGTGGGACAAGGGCAGAGACTTCGCCACCGTGA |
| | | GAAAGGTGCTGAGCATGCCCCAGGTGAACATCGTGAAGAAGACCGAGGTGCAGACCGGC |
| | | GGCTTCAGCAAGGAGAGCATCCTGCCCAAGAGAAACAGCGACAAGCTGATCGCCAGAAA |
| | | GAAGGACTGGGACCCCAAGAAGTACGGCGGCTTCGACAGCCCCACCGTGGCCTACAGCG |
| | | TGCTGGTGGTGGCCAAGGTGGAGAAGGGCAAGAGCAAGAAGCTGAAGAGTGTGAAGGAG |
| | | CTGCTGGGCATCACCATCATGGAGAGAAGCAGCTTCGAGAAGAACCCCATCGACTTCCT |
| | | GGAGGCCAAGGGCTACAAGGAGGTGAAGAAGGACCTGATCATCAAGCTGCCCAAGTACA |
| | | GCCTGTTCGAGCTGGAGAACGGCAGAAAGAGAATGCTGGCCAGCGCCGGCGAGCTGCAG |
| | | AAGGGCAACGAGCTGGCCCTGCCCAGCAAGTACGTGAACTTCCTGTACCTGGCCAGCCA |
| | | CTACGAGAAGCTGAAGGGCAGCCCCGAGGACAACGAGCAGAAGCAGCTGTTCGTGGAGC |
| | | AGCACAAGCACTACCTGGACGAGATCATCGAGCAGATCAGCGAGTTCAGCAAGAGAGTG |
| | | ATCCTGGCCGACGCCAACCTGGACAAGGTGCTGAGCGCCTACAACAAGCACAGAGACAA |
| | | GCCCATCAGAGAGCAGGCCGAGAACATCATCCACCTGTTCACCCTGACCAACCTGGGCG |
| | | CCCCCGCCGCCTTCAAGTACTTCGACACCACCATCGACAGAAAGAGATACACCAGCACC |
| | | AAGGAGGTGCTGGACGCCACCCTGATCCACCAGAGCATCACCGGCCTGTACGAGACCAG |
| | | AATCGACCTGAGCCAGCTGGGGGGCGACAGCGGCGGCAGCAGCGGCGGCAGCAGCGGCA |
| | | GCGAGACCCCCGGCACCAGCGAGAGCGCCACCCCCGAGAGCAGCGGCGGCAGCAGCGGC |
| | | GGCAGCAGCACCCTGAACATCGAGGACGAGTACAGACTGCACGAGACCAGCAAGGAGCC |
| | | CGACGTGAGCCTGGGCAGCACCTGGCTGAGCGACTTCCCCCAGGCCTGGGCCGAGACCG |
| | | GCGGCATGGGCCTGGCCGTGAGACAGGCCCCCCTGATCATCCCCCTGAAGGCCACCAGC |
| | | ACCCCCGTGAGCATCAAGCAGTACCCCATGAGCCAGGAGGCCAGACTGGGCATCAAGCC |
| | | CCACATCCAGAGACTGCTGGACCAGGGCATCCTGGTGCCCTGCCAGAGCCCCTGGAACA |
| | | CCCCCCTGCTGCCCGTGAAGAAGCCCGGCACCAACGACTACAGACCCGTGCAGGACCTG |
| | | AGAGAGGTGAACAAGAGAGTGGAGGACATCCACCCCACCGTGCCCAACCCCTACAACCT |
| | | GCTGAGCGGCCTGCCCCCCAGCCACCAGTGGTACACCGTGCTGGACCTGAAGGACGCCT |
| | | TCTTCTGCCTGAGACTGCACCCCACCAGCCAGCCCCTGTTCGCCTTCGAGTGGAGAGAC |
| | | CCCGAGATGGGCATCAGCGGCCAGCTGACCTGGACCAGACTGCCCCAGGGCTTCAAGAA |
| | | CAGCCCCACCCTGTTCAACGAGGCCCTGCACAGAGACCTGGCCGACTTCAGAATCCAGC |
| | | ACCCCGACCTGATCCTGCTGCAGTACGTGGACGACCTGCTGCTGGCCGCCACCAGCGAG |
| | | CTGGACTGCCAGCAGGGCACCAGAGCCCTGCTGCAGACCCTGGGCAACCTGGGCTACAG |
| | | AGCCAGCGCCAAGAAGGCCCAGATCTGCCAGAAGCAGGTGAAGTACCTGGGCTACCTGC |
| | | TGAAGGAGGGCCAGAGATGGCTGACCGAGGCCAGAAAGGAGACCGTGATGGGCCAGCCC |
| | | ACCCCCAAGACCCCCAGACAGCTGAGAGAGTTCCTGGGCAAGGCCGGCTTCTGCAGACT |
| | | GTTCATCCCCGGCTTCGCCGAGATGGCCGCCCCCCTGTACCCCCTGACCAAGCCCGGCA |
| | | CCCTGTTCAACTGGGGCCCCGACCAGCAGAAGGCCTACCAGGAGATCAAGCAGGCCCTG |
| | | CTGACCGCCCCCGCCCTGGGCCTGCCCGACCTGACCAAGCCCTTCGAGCTGTTCGTGGA |
| | | CGAGAAGCAGGGCTACGCCAAGGGCGTGCTGACCCAGAAGCTGGGCCCCTGGAGAAGAC |
| | | CCGTGGCCTACCTGAGCAAGAAGCTGGACCCCGTGGCCGCCGGCTGGCCCCCCTGCCTG |
| | | AGAATGGTGGCCGCCATCGCCGTGCTGACCAAGGACGCCGGCAAGCTGACCATGGGCCA |
| | | GCCCCTGGTGATCCTGGCCCCCCACGCCGTGGAGGCCCTGGTGAAGCAGCCCCCCGACA |
| | | GATGGCTGAGCAACGCCAGAATGACCCACTACCAGGCCCTGCTGCTGGACACCGACAGA |
| | | GTGCAGTTCGGCCCCGTGGTGGCCCTGAACCCCGCCACCCTGCTGCCCCTGCCCGAGGA |
| | | GGGCCTGCAGCACAACTGCCTGGACATCCTGGCCGAGGCCCACGGCACCAGACCCGACC |
| | | TGACCGACCAGCCCCTGCCCGACGCCGACCACACCTGGTACACCGACGGCAGCAGCCTG |
| | | CTGCAGGAGGGCCAGAGAAAGGCCGGCGCCGCCGTGACCACCGAGACCGAGGTGATCTG |
| | | GGCCAAGGCCCTGCCCGCCGGCACCAGCGCCCAGAGAGCCGAGCTGATCGCCCTGACCC |
| | | AGGCCCTGAAGATGGCCGAGGGCAAGAAGCTGAACGTGTACACCGACAGCAGATACGCC |
| | | TTCGCCACCGCCCACATCCACGGCGAGATCTACAGAAGAAGAGGCTGGCTGACCAGCGA |
| | | GGGCAAGGAGATCAAGAACAAGGACGAGATCCTGGCCCTGCTGAAGGCCCTGTTCCTGC |
| | | CCAAGAGACTGAGCATCATCCACTGCCCCGGCCACCAGAAGGGCCACAGCGCCGAGGCC |
| | | AGAGGCAACAGAATGGCCGACCAGGCCGCCAGAAAGGCCGCCATCACCGAGACCCCCGA |
| | | CACCAGCACCCTGCTGATCGAGAACAGCAGCCCCAGCGGCGGCAGCAAGAGAACCGCCG |
| | | ACGGCAGCGAGTTCGAGCCCAAGAAGAAGAGAAAGGTGGGCAGCGGCAAGGACAAGAAA |
| | | TCCGATGGCAAGGACAGCCAGAAAAAATGA |
| 647 | PL1926 | AAAAAAAAAAAAAAAAAAAAAAAAAAAAAAAAAAAAAAAAAAtgaagagcaggcctgca |
| | | tgcaagcttctagcataaccccttggggcctctaaacgggtcttgaggggttttttggg |
| | | tgtaatcatggtcatagctgtttcctgtgtgaacagcctggggctcaccttcggggggc |
| | | ctttctgcgggtgcctaatgagtgagctaactcacattaattgcgttgcgctcactgcc |
| | | cgctttccagtcgggaaacctgtcgtgccagctgcattaatgaatcggccaacgcgcgg |
| | | ggagaggcggtttgcgtattgggcgccgaagaaaggcccacccgtgaaggtgagcctct |
| | | tccgcttcctcgctcactgactcgctgcgctcggtcgttcggctgcggcgagcggtatc |
| | | agctcactcaaaggcggtaatacggttatccacagaatcaggggataacgcaggaaaga |
| | | acatgtgagcaaaaggccagcaaaaggccaggaaccgtaaaaaggccgcgttgctggcg |
| | | tttttccataggctccgcccccctgacgagcatcacaaaaatcgacgctcaagtcagag |
| | | gtggcgaaacccgacaggactataaagataccaggcgtttccccctggaagctccctcg |
| | | tgcgctctcctgttccgaccctgccgcttaccggatacctgtccgcctttctcccttcg |
| | | ggaagcgtggcgctttctcatagctcacgctgtaggtatctcagttcggtgtaggtcgt |
| | | tcgctccaagctgggctgtgtgcacgaaccccccgttcagcccgaccgctgcgccttat |
| | | ccggtaactatcgtcttgagtccaacccggtaagacacgacttatcgccactggcagca |
| | | gccactggtaacaggattagcagagcgaggtatgtaggcggtgctacagagttcttgaa |
| | | gtggtggcctaactacggctacactagaagaacagtatttggtatctgcgctctgctga |
| | | agccagttaccttcggaaaaagagttggtagctcttgatccggcaaacaaaccaccgct |
| | | ggtagcggtggtttttttgtttgcaagcagcagattacgcgcagaaaaaaaggatctca |
| | | agaagatcctttgatcttttctacggggtctgacgctcagtggaacgaaaactcacgtt |
| | | aagggattttggtcatgagattatcaaaaaggatcttcacctagatccttttaaattaa |
| | | aaatgaagttttaaatcaagcccaatctgaataatgttacaaccaattaaccaattctg |
| | | attagaaaaactcatcgagcatcaaatgaaactgcaatttattcatatcaggattatca |
| | | ataccatatttttgaaaaagccgtttctgtaatgaaggagaaaactcaccgaggcagtt |
| | | ccataggatggcaagatcctggtatcggtctgcgattccgactcgtccaacatcaatac |
| | | aacctattaatttcccctcgtcaaaaataaggttatcaagtgagaaatcaccatgagtg |
| | | acgactgaatccggtgagaatggcaaaagtttatgcatttctttccagacttgttcaac |
| | | aggccagccattacgctcgtcatcaaaatcactcgcatcaaccaaaccgttattcattc |
| | | gtgattgcgcctgagcgagacgaaatacgcgatcgctgttaaaaggacaattacaaaca |
| | | ggaatcgaatgcaaccggcgcaggaacactgccagcgcatcaacaatattttcacctga |
| | | atcaggatattcttctaatacctggaatgctgtttttccggggatcgcagtggtgagta |
| | | accatgcatcatcaggagtacggataaaatgcttgatggtcggaagaggcataaattcc |
| | | gtcagccagtttagtctgaccatctcatctgtaacatcattggcaacgctacctttgcc |
| | | atgtttcagaaacaactctggcgcatcgggcttcccatacaagcgatagattgtcgcac |
| | | ctgattgcccgacattatcgcgagcccatttatacccatataaatcagcatccatgttg |
| | | gaatttaatcgcggcctcgacgtttcccgttgaatatggctcataacaccccttgtatt |
| | | actgtttatgtaagcagacagttttattgttcatgatgatatatttttatcttgtgcaa |
| | | tgtaacatcagagattttgagacacgggccagagctgcatcgcgcgtttcggtgatgac |
| | | ggtgaaaacctctgacacatgcagctcccggagacggtcacagcttgtctgtaagcgga |
| | | tgccgggagcagacaagcccgtcagggcgcgtcagcgggtgttggcgggtgtcggggct |
| | | ggcttaactatgcggcatcagagcagattgtactgagagtgcaccatatgcggtgtgaa |
| | | ataccgcacagatgcgtaaggagaaaataccgcatcaggcgccattcgccattcaggct |
| | | gcgcaactgttgggaagggcgatcggtgcgggcctcttcgctattacgccagctggcga |
| | | aagggggatgtgctgcaaggcgattaagttgggtaacgccagggttttcccagtcacga |
| | | cgttgtaaaacgacggccagagaattcgagctcggtaccTAATACGACTCACTATAaGG |
| | | AAGCTCAGAATAAACGCTCAACTTTGGCCGGATCTGCCACCATGAAGAGAACCGCCGAC |
| | | GGCAGCGAGTTCGAGAGCCCCAAGAAGAAGAGAAAGGTGCTGAACATCGAGGACGAGTA |
| | | CAGACTGCACGAGACCAGCAAGGAGCCCGACGTGAGCCTGGGCAGCACCTGGCTGAGCG |
| | | ACTTCCCCCAGGCCTGGGCCGAGACCGGCGGCATGGGCCTGGCCGTGAGACAGGCCCCC |
| | | CTGATCATCCCCCTGAAGGCCACCAGCACCCCCGTGAGCATCAAGCAGTACCCCATGAG |
| | | CCAGGAGGCCAGACTGGGCATCAAGCCCCACATCCAGAGACTGCTGGACCAGGGCATCC |
| | | TGGTGCCCTGCCAGAGCCCCTGGAACACCCCCCTGCTGCCCGTGAAGAAGCCCGGCACC |
| | | AACGACTACAGACCCGTGCAGGACCTGAGAGAGGTGAACAAGAGAGTGGAGGACATCCA |
| | | CCCCACCGTGCCCAACCCCTACAACCTGCTGAGCGGCCTGCCCCCCAGCCACCAGTGGT |
| | | ACACCGTGCTGGACCTGAAGGACGCCTTCTTCTGCCTGAGACTGCACCCCACCAGCCAG |
| | | CCCCTGTTCGCCTTCGAGTGGAGAGACCCCGAGATGGGCATCAGCGGCCAGCTGACCTG |
| | | GACCAGACTGCCCCAGGGCTTCAAGAACAGCCCCACCCTGTTCAACGAGGCCCTGCACA |
| | | GAGACCTGGCCGACTTCAGAATCCAGCACCCCGACCTGATCCTGCTGCAGTACGTGGAC |
| | | GACCTGCTGCTGGCCGCCACCAGCGAGCTGGACTGCCAGCAGGGCACCAGAGCCCTGCT |
| | | GCAGACCCTGGGCAACCTGGGCTACAGAGCCAGCGCCAAGAAGGCCCAGATCTGCCAGA |
| | | AGCAGGTGAAGTACCTGGGCTACCTGCTGAAGGAGGGCCAGAGATGGCTGACCGAGGCC |
| | | AGAAAGGAGACCGTGATGGGCCAGCCCACCCCCAAGACCCCCAGACAGCTGAGAGAGTT |
| | | CCTGGGCAAGGCCGGCTTCTGCAGACTGTTCATCCCCGGCTTCGCCGAGATGGCCGCCC |
| | | CCCTGTACCCCCTGACCAAGCCCGGCACCCTGTTCAACTGGGGCCCCGACCAGCAGAAG |
| | | GCCTACCAGGAGATCAAGCAGGCCCTGCTGACCGCCCCCGCCCTGGGCCTGCCCGACCT |
| | | GACCAAGCCCTTCGAGCTGTTCGTGGACGAGAAGCAGGGCTACGCCAAGGGCGTGCTGA |
| | | CCCAGAAGCTGGGCCCCTGGAGAAGACCCGTGGCCTACCTGAGCAAGAAGCTGGACCCC |
| | | GTGGCCGCCGGCTGGCCCCCCTGCCTGAGAATGGTGGCCGCCATCGCCGTGCTGACCAA |
| | | GGACGCCGGCAAGCTGACCATGGGCCAGCCCCTGGTGATCCTGGCCCCCCACGCCGTGG |
| | | AGGCCCTGGTGAAGCAGCCCCCCGACAGATGGCTGAGCAACGCCAGAATGACCCACTAC |
| | | CAGGCCCTGCTGCTGGACACCGACAGAGTGCAGTTCGGCCCCGTGGTGGCCCTGAACCC |
| | | CGCCACCCTGCTGCCCCTGCCCGAGGAGGGCCTGCAGCACAACTGCCTGGACATCCTGG |
| | | CCGAGGCCCACGGCACCAGACCCGACCTGACCGACCAGCCCCTGCCCGACGCCGACCAC |
| | | ACCTGGTACACCGACGGCAGCAGCCTGCTGCAGGAGGGCCAGAGAAAGGCCGGCGCCGC |
| | | CGTGACCACCGAGACCGAGGTGATCTGGGCCAAGGCCCTGCCCGCCGGCACCAGCGCCC |
| | | AGAGAGCCGAGCTGATCGCCCTGACCCAGGCCCTGAAGATGGCCGAGGGCAAGAAGCTG |
| | | AACGTGTACACCGACAGCAGATACGCCTTCGCCACCGCCCACATCCACGGCGAGATCTA |
| | | CAGAAGAAGAGGCTGGCTGACCAGCGAGGGCAAGGAGATCAAGAACAAGGACGAGATCC |
| | | TGGCCCTGCTGAAGGCCCTGTTCCTGCCCAAGAGACTGAGCATCATCCACTGCCCCGGC |
| | | CACCAGAAGGGCCACAGCGCCGAGGCCAGAGGCAACAGAATGGCCGACCAGGCCGCCAG |
| | | AAAGGCCGCCATCACCGAGACCCCCGACACCAGCACCCTGCTGATCGAGAACAGCAGCC |
| | | CCAGCGGCGGCAGCAAGAGAACCGCCGACGGCAGCGAGTTCGAGCCCAAGAAGAAGAGA |
| | | AAGGTGGGCAGCGGCAAGGACAAGAAATCCGATGGCAAGGACAGCCAGAAAAAATAGTG |
| | | Aacatggggggtctgcggacggcttcggcccacccgcgacaagaatgccgtcatctgtc |
| | | ctcattacccgtattccttcccttcccccgcaaccaccacgcttactcgcgcacgtgtt |
| | | gagtggcacgtgcgttgtccaaacagctacacccacacccttcggggcgggtttgtccc |
| | | gccctcgggttcctcgcggaacccccccctccctctctctctttctatccgccctcact |
| | | tcccataactacagtgctttggtaggtgagcaccctgaccccccgcggaagctgctaac |
| | | gtggcaactgtggggatccaggcaggttatcaaaggcacccggtctttccgccttcagg |
| | | agtatctctgccggtgaattccggtagggctctgcttggtgccaacctcccccaaatgc |
| | | gcgctgcgggagtgcActtccccaactcatcttagtaacctctcatgtgtgtgcttggt |
| | | cagcatatctgaggcgacgttccgctgtcccagaccagtccagcaatggacgggccagt |
| | | gtgcgtagtcgctttccggttttccggcgcatgtttggcgaaacgctgaggtaaggttg |
| | | gtgtgcccaacgcccgtaatttggtgatacctcaagaccacccaggaatgccagggagg |
| | | taccccacttcggtgggatctgaccctgggctaattgtctacggtggttcttcttgctt |
| | | ccacttctcttttttctggcatgAAGAGAACCGCCGACGGCAGCGAGTTCGAGAGCCCC |
| | | AAGAAGAAGAGAAAGGTGGACAAGAAGTACAGCATCGGCCTGGACATCGGCACCAACAG |
| | | CGTGGGCTGGGCCGTGATCACCGACGAGTACAAGGTGCCCAGCAAGAAGTTCAAGGTGC |
| | | TGGGCAACACCGACAGACACAGCATCAAGAAGAACCTGATCGGCGCCCTGCTGTTCGAC |
| | | AGCGGCGAGACCGCCGAGGCCACCAGACTGAAGAGAACCGCCAGAAGAAGATACACCAG |
| | | AAGAAAGAACAGAATCTGCTACCTGCAGGAGATCTTCAGCAACGAGATGGCCAAGGTGG |
| | | ACGACAGCTTCTTCCACAGACTGGAGGAGAGCTTCCTGGTGGAGGAGGACAAGAAGCAC |
| | | GAGAGACACCCCATCTTCGGCAACATCGTGGACGAGGTGGCCTACCACGAGAAGTACCC |
| | | CACCATCTACCACCTGAGAAAGAAGCTGGTGGACAGCACCGACAAGGCCGACCTGAGAC |
| | | TGATCTACCTGGCCCTGGCCCACATGATCAAGTTCAGAGGCCACTTCCTGATCGAGGGC |
| | | GACCTGAACCCCGACAACAGCGACGTGGACAAGCTGTTCATCCAGCTGGTGCAGACCTA |
| | | CAACCAGCTGTTCGAGGAGAACCCCATCAACGCCAGCGGCGTGGACGCCAAGGCCATCC |
| | | TGAGCGCCAGACTGAGCAAGAGCAGAAGACTGGAGAACCTGATCGCCCAGCTGCCCGGC |
| | | GAGAAGAAGAACGGCCTGTTCGGCAACCTGATCGCCCTGAGCCTGGGCCTGACCCCCAA |
| | | CTTCAAGAGCAACTTCGACCTGGCCGAGGACGCCAAGCTGCAGCTGAGCAAGGACACCT |
| | | ACGACGACGACCTGGACAACCTGCTGGCCCAGATCGGCGACCAGTACGCCGACCTGTTC |
| | | CTGGCCGCCAAGAACCTGAGCGACGCCATCCTGCTGAGCGACATCCTGAGAGTGAACAC |
| | | CGAGATCACCAAGGCCCCCCTGAGCGCCAGCATGATCAAGAGATACGACGAGCACCACC |
| | | AGGACCTGACCCTGCTGAAGGCCCTGGTGAGACAGCAGCTGCCCGAGAAGTACAAGGAG |
| | | ATCTTCTTCGACCAGAGCAAGAACGGCTACGCCGGCTACATCGACGGCGGCGCCAGCCA |
| | | GGAGGAGTTCTACAAGTTCATCAAGCCCATCCTGGAGAAGATGGACGGCACCGAGGAGC |
| | | TGCTGGTGAAGCTGAACAGAGAGGACCTGCTGAGAAAGCAGAGAACCTTCGACAACGGC |
| | | AGCATCCCCCACCAGATCCACCTGGGCGAGCTGCACGCCATCCTGAGAAGACAGGAGGA |
| | | CTTCTACCCCTTCCTGAAGGACAACAGAGAGAAGATCGAGAAGATCCTGACCTTCAGAA |
| | | TCCCCTACTACGTGGGCCCCCTGGCCAGAGGCAACAGCAGATTCGCCTGGATGACCAGA |
| | | AAGAGCGAGGAGACCATCACCCCCTGGAACTTCGAGGAGGTGGTGGACAAGGGCGCCAG |
| | | CGCCCAGAGCTTCATCGAGAGAATGACCAACTTCGACAAGAACCTGCCCAACGAGAAGG |
| | | TGCTGCCCAAGCACAGCCTGCTGTACGAGTACTTCACCGTGTACAACGAGCTGACCAAG |
| | | GTGAAGTACGTGACCGAGGGCATGAGAAAGCCCGCCTTCCTGAGCGGCGAGCAGAAGAA |
| | | GGCCATCGTGGACCTGCTGTTCAAGACCAACAGAAAGGTGACCGTGAAGCAGCTGAAGG |
| | | AGGACTACTTCAAGAAGATCGAGTGCTTCGACAGCGTGGAGATCAGCGGCGTGGAGGAC |
| | | AGATTCAACGCCAGCCTGGGCACCTACCACGACCTGCTGAAGATCATCAAGGACAAGGA |
| | | CTTCCTGGACAACGAGGAGAACGAGGACATCCTGGAGGACATCGTGCTGACCCTGACCC |
| | | TGTTCGAGGACAGAGAGATGATCGAGGAGAGACTGAAGACCTACGCCCACCTGTTCGAC |
| | | GACAAGGTGATGAAGCAGCTGAAGAGAAGAAGATACACCGGCTGGGGCAGACTGAGCAG |
| | | AAAGCTGATCAACGGCATCAGAGACAAGCAGAGCGGCAAGACCATCCTGGACTTCCTGA |
| | | AGAGTGACGGCTTCGCCAACAGAAACTTCATGCAGCTGATCCACGACGACAGCCTGACC |
| | | TTCAAGGAGGACATCCAGAAGGCCCAGGTGAGCGGCCAGGGCGACAGCCTGCACGAGCA |
| | | CATCGCCAACCTGGCCGGCAGCCCCGCCATCAAGAAGGGCATCCTGCAGACCGTGAAGG |
| | | TGGTGGACGAGCTGGTGAAGGTGATGGGCAGACACAAGCCCGAGAACATCGTGATCGAG |
| | | ATGGCCAGAGAGAACCAGACCACCCAGAAGGGCCAGAAGAACAGCAGAGAGAGAATGAA |
| | | GAGAATCGAGGAGGGCATCAAGGAGCTGGGCAGCCAGATCCTGAAGGAGCACCCCGTGG |
| | | AGAACACCCAGCTGCAGAACGAGAAGCTGTACCTGTACTACCTGCAGAACGGCAGAGAC |
| | | ATGTACGTGGACCAGGAGCTGGACATCAACAGACTGAGCGACTACGACGTGGACGCCAT |
| | | CGTGCCCCAGAGCTTCCTGAAGGACGACAGCATCGACAACAAGGTGCTGACCAGAAGCG |
| | | ACAAGAACAGAGGCAAGAGCGACAACGTGCCCAGCGAGGAGGTGGTGAAGAAGATGAAG |
| | | AACTACTGGAGACAGCTGCTGAACGCCAAGCTGATCACCCAGAGAAAGTTCGACAACCT |
| | | GACCAAGGCCGAGAGAGGCGGCCTGAGCGAGCTGGACAAGGCCGGCTTCATCAAGAGAC |
| | | AGCTGGTGGAGACCAGACAGATCACCAAGCACGTGGCCCAGATCCTGGACAGCAGAATG |
| | | AACACCAAGTACGACGAGAACGACAAGCTGATCAGAGAGGTGAAGGTGATCACCCTGAA |
| | | GAGTAAGCTGGTGAGCGACTTCAGAAAGGACTTCCAGTTCTACAAGGTGAGAGAGATCA |
| | | ACAACTACCACCACGCCCACGACGCCTACCTGAACGCCGTGGTGGGCACCGCCCTGATC |
| | | AAGAAGTACCCCAAGCTGGAGAGCGAGTTCGTGTACGGCGACTACAAGGTGTACGACGT |
| | | GAGAAAGATGATCGCCAAGAGCGAGCAGGAGATCGGCAAGGCCACCGCCAAGTACTTCT |
| | | TCTACAGCAACATCATGAACTTCTTCAAGACCGAGATCACCCTGGCCAACGGCGAGATC |
| | | AGAAAGAGACCCCTGATCGAGACCAACGGCGAGACCGGCGAGATCGTGTGGGACAAGGG |
| | | CAGAGACTTCGCCACCGTGAGAAAGGTGCTGAGCATGCCCCAGGTGAACATCGTGAAGA |
| | | AGACCGAGGTGCAGACCGGCGGCTTCAGCAAGGAGAGCATCCTGCCCAAGAGAAACAGC |
| | | GACAAGCTGATCGCCAGAAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGA |
| | | AGCTGAAGAGTGTGAAGGAGCTGCTGGGCATCACCATCATGGAGAGAAGCAGCTTCGAG |
| | | AAGAACCCCATCGACTTCCTGGAGGCCAAGGGCTACAAGGAGGTGAAGAAGGACCTGAT |
| | | CATCAAGCTGCCCAAGTACAGCCTGTTCGAGCTGGAGAACGGCAGAAAGAGAATGCTGG |
| | | CCAGCGCCGGCGAGCTGCAGAAGGGCAACGAGCTGGCCCTGCCCAGCAAGTACGTGAAC |
| | | TTCCTGTACCTGGCCAGCCACTACGAGAAGCTGAAGGGCAGCCCCGAGGACAACGAGCA |
| | | GAAGCAGCTGTTCGTGGAGCAGCACAAGCACTACCTGGACGAGATCATCGAGCAGATCA |
| | | GCGAGTTCAGCAAGAGAGTGATCCTGGCCGACGCCAACCTGGACAAGGTGCTGAGCGCC |
| | | TACAACAAGCACAGAGACAAGCCCATCAGAGAGCAGGCCGAGAACATCATCCACCTGTT |
| | | CACCCTGACCAACCTGGGCGCCCCCGCCGCCTTCAAGTACTTCGACACCACCATCGACA |
| | | GAAAGAGATACACCAGCACCAAGGAGGTGCTGGACGCCACCCTGATCCACCAGAGCATC |
| | | ACCGGCCTGTACGAGACCAGAATCGACCTGAGCCAGCTGGGGGGCGACGGAGGAGGAAG |
| | | CCCGAAGAAGAAGAGAAAGGTCTAGTGAACCAGCCTCAAGAACACCCGAATGGAGTCTC |
| | | TAAGCTACATAATACCAACTTACACTTTACAAAATGTTGTCCCCCAAAATGTAGCCATT |
| | | CGTATCTGCTCCTAATAAAAAGAAAGTTTCTTCACATTCTAAAAAAAAAAAAAAAAAAA |
| | | AAAAAAAAAAAAAAAAAAA |
| 648 | PL1927 | AAAAAAAAAAAAAAAAAAAAAAAAAAAAAAAAAAAAAAAAAAAtgaagagcaggcctgc |
| | | atgcaagcttctagcataaccccttggggcctctaaacgggtcttgaggggttttttgg |
| | | gtgtaatcatggtcatagctgtttcctgtgtgaacagcctggggctcaccttcgggggg |
| | | cctttctgcgggtgcctaatgagtgagctaactcacattaattgcgttgcgctcactgc |
| | | ccgctttccagtcgggaaacctgtcgtgccagctgcattaatgaatcggccaacgcgcg |
| | | gggagaggcggtttgcgtattgggcgccgaagaaaggcccacccgtgaaggtgagcctc |
| | | ttccgcttcctcgctcactgactcgctgcgctcggtcgttcggctgcggcgagcggtat |
| | | cagctcactcaaaggcggtaatacggttatccacagaatcaggggataacgcaggaaag |
| | | aacatgtgagcaaaaggccagcaaaaggccaggaaccgtaaaaaggccgcgttgctggc |
| | | gtttttccataggctccgcccccctgacgagcatcacaaaaatcgacgctcaagtcaga |
| | | ggtggcgaaacccgacaggactataaagataccaggcgtttccccctggaagctccctc |
| | | gtgcgctctcctgttccgaccctgccgcttaccggatacctgtccgcctttctcccttc |
| | | gggaagcgtggcgctttctcatagctcacgctgtaggtatctcagttcggtgtaggtcg |
| | | ttcgctccaagctgggctgtgtgcacgaaccccccgttcagcccgaccgctgcgcctta |
| | | tccggtaactatcgtcttgagtccaacccggtaagacacgacttatcgccactggcagc |
| | | agccactggtaacaggattagcagagcgaggtatgtaggcggtgctacagagttcttga |
| | | agtggtggcctaactacggctacactagaagaacagtatttggtatctgcgctctgctg |
| | | aagccagttaccttcggaaaaagagttggtagctcttgatccggcaaacaaaccaccgc |
| | | tggtagcggtggtttttttgtttgcaagcagcagattacgcgcagaaaaaaaggatctc |
| | | aagaagatcctttgatcttttctacggggtctgacgctcagtggaacgaaaactcacgt |
| | | taagggattttggtcatgagattatcaaaaaggatcttcacctagatccttttaaatta |
| | | aaaatgaagttttaaatcaagcccaatctgaataatgttacaaccaattaaccaattct |
| | | gattagaaaaactcatcgagcatcaaatgaaactgcaatttattcatatcaggattatc |
| | | aataccatatttttgaaaaagccgtttctgtaatgaaggagaaaactcaccgaggcagt |
| | | tccataggatggcaagatcctggtatcggtctgcgattccgactcgtccaacatcaata |
| | | caacctattaatttcccctcgtcaaaaataaggttatcaagtgagaaatcaccatgagt |
| | | gacgactgaatccggtgagaatggcaaaagtttatgcatttctttccagacttgttcaa |
| | | caggccagccattacgctcgtcatcaaaatcactcgcatcaaccaaaccgttattcatt |
| | | cgtgattgcgcctgagcgagacgaaatacgcgatcgctgttaaaaggacaattacaaac |
| | | aggaatcgaatgcaaccggcgcaggaacactgccagcgcatcaacaatattttcacctg |
| | | aatcaggatattcttctaatacctggaatgctgtttttccggggatcgcagtggtgagt |
| | | aaccatgcatcatcaggagtacggataaaatgcttgatggtcggaagaggcataaattc |
| | | cgtcagccagtttagtctgaccatctcatctgtaacatcattggcaacgctacctttgc |
| | | catgtttcagaaacaactctggcgcatcgggcttcccatacaagcgatagattgtcgca |
| | | cctgattgcccgacattatcgcgagcccatttatacccatataaatcagcatccatgtt |
| | | ggaatttaatcgcggcctcgacgtttcccgttgaatatggctcataacaccccttgtat |
| | | tactgtttatgtaagcagacagttttattgttcatgatgatatatttttatcttgtgca |
| | | atgtaacatcagagattttgagacacgggccagagctgcatcgcgcgtttcggtgatga |
| | | cggtgaaaacctctgacacatgcagctcccggagacggtcacagcttgtctgtaagcgg |
| | | atgccgggagcagacaagcccgtcagggcgcgtcagcgggtgttggcgggtgtcggggc |
| | | tggcttaactatgcggcatcagagcagattgtactgagagtgcaccatatgcggtgtga |
| | | aataccgcacagatgcgtaaggagaaaataccgcatcaggcgccattcgccattcaggc |
| | | tgcgcaactgttgggaagggcgatcggtgcgggcctcttcgctattacgccagctggcg |
| | | aaagggggatgtgctgcaaggcgattaagttgggtaacgccagggttttcccagtcacg |
| | | acgttgtaaaacgacggccagagaattcgagctcggtaccTAATACGACTCACTATAaG |
| | | GAAGCTCAGAATAAACGCTCAACTTTGGCCGGATCTGCCACCATGAAGAGAACCGCCGA |
| | | CGGCAGCGAGTTCGAGAGCCCCAAGAAGAAGAGAAAGGTGCTGAACATCGAGGACGAGT |
| | | ACAGACTGCACGAGACCAGCAAGGAGCCCGACGTGAGCCTGGGCAGCACCTGGCTGAGC |
| | | GACTTCCCCCAGGCCTGGGCCGAGACCGGCGGCATGGGCCTGGCCGTGAGACAGGCCCC |
| | | CCTGATCATCCCCCTGAAGGCCACCAGCACCCCCGTGAGCATCAAGCAGTACCCCATGA |
| | | GCCAGGAGGCCAGACTGGGCATCAAGCCCCACATCCAGAGACTGCTGGACCAGGGCATC |
| | | CTGGTGCCCTGCCAGAGCCCCTGGAACACCCCCCTGCTGCCCGTGAAGAAGCCCGGCAC |
| | | CAACGACTACAGACCCGTGCAGGACCTGAGAGAGGTGAACAAGAGAGTGGAGGACATCC |
| | | ACCCCACCGTGCCCAACCCCTACAACCTGCTGAGCGGCCTGCCCCCCAGCCACCAGTGG |
| | | TACACCGTGCTGGACCTGAAGGACGCCTTCTTCTGCCTGAGACTGCACCCCACCAGCCA |
| | | GCCCCTGTTCGCCTTCGAGTGGAGAGACCCCGAGATGGGCATCAGCGGCCAGCTGACCT |
| | | GGACCAGACTGCCCCAGGGCTTCAAGAACAGCCCCACCCTGTTCAACGAGGCCCTGCAC |
| | | AGAGACCTGGCCGACTTCAGAATCCAGCACCCCGACCTGATCCTGCTGCAGTACGTGGA |
| | | CGACCTGCTGCTGGCCGCCACCAGCGAGCTGGACTGCCAGCAGGGCACCAGAGCCCTGC |
| | | TGCAGACCCTGGGCAACCTGGGCTACAGAGCCAGCGCCAAGAAGGCCCAGATCTGCCAG |
| | | AAGCAGGTGAAGTACCTGGGCTACCTGCTGAAGGAGGGCCAGAGATGGCTGACCGAGGC |
| | | CAGAAAGGAGACCGTGATGGGCCAGCCCACCCCCAAGACCCCCAGACAGCTGAGAGAGT |
| | | TCCTGGGCAAGGCCGGCTTCTGCAGACTGTTCATCCCCGGCTTCGCCGAGATGGCCGCC |
| | | CCCCTGTACCCCCTGACCAAGCCCGGCACCCTGTTCAACTGGGGCCCCGACCAGCAGAA |
| | | GGCCTACCAGGAGATCAAGCAGGCCCTGCTGACCGCCCCCGCCCTGGGCCTGCCCGACC |
| | | TGACCAAGCCCTTCGAGCTGTTCGTGGACGAGAAGCAGGGCTACGCCAAGGGCGTGCTG |
| | | ACCCAGAAGCTGGGCCCCTGGAGAAGACCCGTGGCCTACCTGAGCAAGAAGCTGGACCC |
| | | CGTGGCCGCCGGCTGGCCCCCCTGCCTGAGAATGGTGGCCGCCATCGCCGTGCTGACCA |
| | | AGGACGCCGGCAAGCTGACCATGGGCCAGCCCCTGGTGATCCTGGCCCCCCACGCCGTG |
| | | GAGGCCCTGGTGAAGCAGCCCCCCGACAGATGGCTGAGCAACGCCAGAATGACCCACTA |
| | | CCAGGCCCTGCTGCTGGACACCGACAGAGTGCAGTTCGGCCCCGTGGTGGCCCTGAACC |
| | | CCGCCACCCTGCTGCCCCTGCCCGAGGAGGGCCTGCAGCACAACTGCCTGGACATCCTG |
| | | GCCGAGGCCCACGGCACCAGACCCGACCTGACCGACCAGCCCCTGCCCGACGCCGACCA |
| | | CACCTGGTACACCGACGGCAGCAGCCTGCTGCAGGAGGGCCAGAGAAAGGCCGGCGCCG |
| | | CCGTGACCACCGAGACCGAGGTGATCTGGGCCAAGGCCCTGCCCGCCGGCACCAGCGCC |
| | | CAGAGAGCCGAGCTGATCGCCCTGACCCAGGCCCTGAAGATGGCCGAGGGCAAGAAGCT |
| | | GAACGTGTACACCGACAGCAGATACGCCTTCGCCACCGCCCACATCCACGGCGAGATCT |
| | | ACAGAAGAAGAGGCTGGCTGACCAGCGAGGGCAAGGAGATCAAGAACAAGGACGAGATC |
| | | CTGGCCCTGCTGAAGGCCCTGTTCCTGCCCAAGAGACTGAGCATCATCCACTGCCCCGG |
| | | CCACCAGAAGGGCCACAGCGCCGAGGCCAGAGGCAACAGAATGGCCGACCAGGCCGCCA |
| | | GAAAGGCCGCCATCACCGAGACCCCCGACACCAGCACCCTGCTGATCGAGAACAGCAGC |
| | | CCCAGCGGCGGCAGCAAGAGAACCGCCGACGGCAGCGAGTTCGAGCCCAAGAAGAAGAG |
| | | AAAGGTGGGCAGCGGCAAGGACAAGAAATCCGATGGCAAGGACAGCCAGAAAAAATAGT |
| | | GAACCTAGGCGTAGCGGCCGCAAATTCCGCCCCTCTCCCTCCCCCCCCCCTAACGTTAC |
| | | TGGCCGAAGCCGCTTGGAATAAGGCCGGTGTGCGTTTGTCTATATGTTATTTTCCACCA |
| | | TATTGCCGTCTTTTGGCAATGTGAGGGCCCGGAAACCTGGCCCTGTCTTCTTGACGAGC |
| | | ATTCCTAGGGGTCTTTCCCCTCTCGCCAAAGGAATGCAAGGTCTGTTGAATGTCGTGAA |
| | | GGAAGCAGTTCCTCTGGAAGCTTCTTGAAGACAAACAACGTCTGTAGCGACCCTTTGCA |
| | | GGCAGCGGAACCCCCCACCTGGCGACAGGTGCCTCTGCGGCCAAAAGCCACGTGTATAA |
| | | GATACACCTGCAAAGGCGGCACAACCCCAGTGCCACGTTGTGAGTTGGATAGTTGTGGA |
| | | AAGAGTCAAATGGCTCTCCTCAAGCGTATTCAACAAGGGGCTGAAGGATGCCCAGAAGG |
| | | TACCCCATTGTATGGGATCTGATCTGGGGCCTCGGTGCACATGCTTTACATGTGTTTAG |
| | | TCGAGGTTAAAAAAACGTCTAGGCCCCCCGAACCACGGGGACGTGGTTTTCCTTTGAAA |
| | | AACACGATGATAATATGAAGAGAACCGCCGACGGCAGCGAGTTCGAGAGCCCCAAGAAG |
| | | AAGAGAAAGGTGGACAAGAAGTACAGCATCGGCCTGGACATCGGCACCAACAGCGTGGG |
| | | CTGGGCCGTGATCACCGACGAGTACAAGGTGCCCAGCAAGAAGTTCAAGGTGCTGGGCA |
| | | ACACCGACAGACACAGCATCAAGAAGAACCTGATCGGCGCCCTGCTGTTCGACAGCGGC |
| | | GAGACCGCCGAGGCCACCAGACTGAAGAGAACCGCCAGAAGAAGATACACCAGAAGAAA |
| | | GAACAGAATCTGCTACCTGCAGGAGATCTTCAGCAACGAGATGGCCAAGGTGGACGACA |
| | | GCTTCTTCCACAGACTGGAGGAGAGCTTCCTGGTGGAGGAGGACAAGAAGCACGAGAGA |
| | | CACCCCATCTTCGGCAACATCGTGGACGAGGTGGCCTACCACGAGAAGTACCCCACCAT |
| | | CTACCACCTGAGAAAGAAGCTGGTGGACAGCACCGACAAGGCCGACCTGAGACTGATCT |
| | | ACCTGGCCCTGGCCCACATGATCAAGTTCAGAGGCCACTTCCTGATCGAGGGCGACCTG |
| | | AACCCCGACAACAGCGACGTGGACAAGCTGTTCATCCAGCTGGTGCAGACCTACAACCA |
| | | GCTGTTCGAGGAGAACCCCATCAACGCCAGCGGCGTGGACGCCAAGGCCATCCTGAGCG |
| | | CCAGACTGAGCAAGAGCAGAAGACTGGAGAACCTGATCGCCCAGCTGCCCGGCGAGAAG |
| | | AAGAACGGCCTGTTCGGCAACCTGATCGCCCTGAGCCTGGGCCTGACCCCCAACTTCAA |
| | | GAGCAACTTCGACCTGGCCGAGGACGCCAAGCTGCAGCTGAGCAAGGACACCTACGACG |
| | | ACGACCTGGACAACCTGCTGGCCCAGATCGGCGACCAGTACGCCGACCTGTTCCTGGCC |
| | | GCCAAGAACCTGAGCGACGCCATCCTGCTGAGCGACATCCTGAGAGTGAACACCGAGAT |
| | | CACCAAGGCCCCCCTGAGCGCCAGCATGATCAAGAGATACGACGAGCACCACCAGGACC |
| | | TGACCCTGCTGAAGGCCCTGGTGAGACAGCAGCTGCCCGAGAAGTACAAGGAGATCTTC |
| | | TTCGACCAGAGCAAGAACGGCTACGCCGGCTACATCGACGGCGGCGCCAGCCAGGAGGA |
| | | GTTCTACAAGTTCATCAAGCCCATCCTGGAGAAGATGGACGGCACCGAGGAGCTGCTGG |
| | | TGAAGCTGAACAGAGAGGACCTGCTGAGAAAGCAGAGAACCTTCGACAACGGCAGCATC |
| | | CCCCACCAGATCCACCTGGGCGAGCTGCACGCCATCCTGAGAAGACAGGAGGACTTCTA |
| | | CCCCTTCCTGAAGGACAACAGAGAGAAGATCGAGAAGATCCTGACCTTCAGAATCCCCT |
| | | ACTACGTGGGCCCCCTGGCCAGAGGCAACAGCAGATTCGCCTGGATGACCAGAAAGAGC |
| | | GAGGAGACCATCACCCCCTGGAACTTCGAGGAGGTGGTGGACAAGGGCGCCAGCGCCCA |
| | | GAGCTTCATCGAGAGAATGACCAACTTCGACAAGAACCTGCCCAACGAGAAGGTGCTGC |
| | | CCAAGCACAGCCTGCTGTACGAGTACTTCACCGTGTACAACGAGCTGACCAAGGTGAAG |
| | | TACGTGACCGAGGGCATGAGAAAGCCCGCCTTCCTGAGCGGCGAGCAGAAGAAGGCCAT |
| | | CGTGGACCTGCTGTTCAAGACCAACAGAAAGGTGACCGTGAAGCAGCTGAAGGAGGACT |
| | | ACTTCAAGAAGATCGAGTGCTTCGACAGCGTGGAGATCAGCGGCGTGGAGGACAGATTC |
| | | AACGCCAGCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACCCTGTTCG |
| | | AGGACAGAGAGATGATCGAGGAGAGACTGAAGACCTACGCCCACCTGTTCGACGACAAG |
| | | GTGATGAAGCAGCTGAAGAGAAGAAGATACACCGGCTGGGGCAGACTGAGCAGAAAGCT |
| | | GATCAACGGCATCAGAGACAAGCAGAGCGGCAAGACCATCCTGGACTTCCTGAAGAGTG |
| | | ACGGCTTCGCCAACAGAAACTTCATGCAGCTGATCCACGACGACAGCCTGACCTTCAAG |
| | | GAGGACATCCAGAAGGCCCAGGTGAGCGGCCAGGGCGACAGCCTGCACGAGCACATCGC |
| | | CAACCTGGCCGGCAGCCCCGCCATCAAGAAGGGCATCCTGCAGACCGTGAAGGTGGTGG |
| | | ACGAGCTGGTGAAGGTGATGGGCAGACACAAGCCCGAGAACATCGTGATCGAGATGGCC |
| | | AGAGAGAACCAGACCACCCAGAAGGGCCAGAAGAACAGCAGAGAGAGAATGAAGAGAAT |
| | | CGAGGAGGGCATCAAGGAGCTGGGCAGCCAGATCCTGAAGGAGCACCCCGTGGAGAACA |
| | | CCCAGCTGCAGAACGAGAAGCTGTACCTGTACTACCTGCAGAACGGCAGAGACATGTAC |
| | | GTGGACCAGGAGCTGGACATCAACAGACTGAGCGACTACGACGTGGACGCCATCGTGCC |
| | | CCAGAGCTTCCTGAAGGACGACAGCATCGACAACAAGGTGCTGACCAGAAGCGACAAGA |
| | | ACAGAGGCAAGAGCGACAACGTGCCCAGCGAGGAGGTGGTGAAGAAGATGAAGAACTAC |
| | | TGGAGACAGCTGCTGAACGCCAAGCTGATCACCCAGAGAAAGTTCGACAACCTGACCAA |
| | | GGCCGAGAGAGGCGGCCTGAGCGAGCTGGACAAGGCCGGCTTCATCAAGAGACAGCTGG |
| | | TGGAGACCAGACAGATCACCAAGCACGTGGCCCAGATCCTGGACAGCAGAATGAACACC |
| | | AAGTACGACGAGAACGACAAGCTGATCAGAGAGGTGAAGGTGATCACCCTGAAGAGTAA |
| | | GCTGGTGAGCGACTTCAGAAAGGACTTCCAGTTCTACAAGGTGAGAGAGATCAACAACT |
| | | ACCACCACGCCCACGACGCCTACCTGAACGCCGTGGTGGGCACCGCCCTGATCAAGAAG |
| | | TACCCCAAGCTGGAGAGCGAGTTCGTGTACGGCGACTACAAGGTGTACGACGTGAGAAA |
| | | GATGATCGCCAAGAGCGAGCAGGAGATCGGCAAGGCCACCGCCAAGTACTTCTTCTACA |
| | | GCAACATCATGAACTTCTTCAAGACCGAGATCACCCTGGCCAACGGCGAGATCAGAAAG |
| | | AGACCCCTGATCGAGACCAACGGCGAGACCGGCGAGATCGTGTGGGACAAGGGCAGAGA |
| | | CTTCGCCACCGTGAGAAAGGTGCTGAGCATGCCCCAGGTGAACATCGTGAAGAAGACCG |
| | | AGGTGCAGACCGGCGGCTTCAGCAAGGAGAGCATCCTGCCCAAGAGAAACAGCGACAAG |
| | | CTGATCGCCAGAAAGAAGGACTGGGACCCCAAGAAGTACGGCGGCTTCGACAGCCCCAC |
| | | CGTGGCCTACAGCGTGCTGGTGGTGGCCAAGGTGGAGAAGGGCAAGAGCAAGAAGCTGA |
| | | AGAGTGTGAAGGAGCTGCTGGGCATCACCATCATGGAGAGAAGCAGCTTCGAGAAGAAC |
| | | CCCATCGACTTCCTGGAGGCCAAGGGCTACAAGGAGGTGAAGAAGGACCTGATCATCAA |
| | | GCTGCCCAAGTACAGCCTGTTCGAGCTGGAGAACGGCAGAAAGAGAATGCTGGCCAGCG |
| | | CCGGCGAGCTGCAGAAGGGCAACGAGCTGGCCCTGCCCAGCAAGTACGTGAACTTCCTG |
| | | TACCTGGCCAGCCACTACGAGAAGCTGAAGGGCAGCCCCGAGGACAACGAGCAGAAGCA |
| | | GCTGTTCGTGGAGCAGCACAAGCACTACCTGGACGAGATCATCGAGCAGATCAGCGAGT |
| | | TCAGCAAGAGAGTGATCCTGGCCGACGCCAACCTGGACAAGGTGCTGAGCGCCTACAAC |
| | | AAGCACAGAGACAAGCCCATCAGAGAGCAGGCCGAGAACATCATCCACCTGTTCACCCT |
| | | GACCAACCTGGGCGCCCCCGCCGCCTTCAAGTACTTCGACACCACCATCGACAGAAAGA |
| | | GATACACCAGCACCAAGGAGGTGCTGGACGCCACCCTGATCCACCAGAGCATCACCGGC |
| | | CTGTACGAGACCAGAATCGACCTGAGCCAGCTGGGCGGCGACGGAGGAGGAAGCCCGAA |
| | | GAAGAAGAGAAAGGTCTAGTGAACCAGCCTCAAGAACACCCGAATGGAGTCTCTAAGCT |
| | | ACATAATACCAACTTACACTTTACAAAATGTTGTCCCCCAAAATGTAGCCATTCGTATC |
| | | TGCTCCTAATAAAAAGAAAGTTTCTTCACATTCTAAAAAAAAAAAAAAAAAAAAAAAAA |
| | | AAAAAAAAAAAA |
| 649 | PL1948 | MPAAKRVKLDYPYDVPDYAKDKKSDGKDSQKKGSRALWIRLSRVTDATTSPERQLESCQ |
| | _AA | QLCAQRGWDVVGVAEDLDVSGAVDPFDRKRRPNLARWLAFEEQPFDVIVAYRVDRLTRS |
| | | IRHLQQLVHWAEDHKKLVVSATEAHFDTTTPFAAVVIALMGTVAQMELEAIKERNRSAA |
| | | HFNIRAGKYRGSLPPWGYLPTRVDGEWRLVPDPVQRERILEVYHRVVDNHEPLHLVAHD |
| | | LNRRGVLSPKDYFAQLQGREPQGREWSATALKRSMISEAMLGYATLNGKTVRDDDGAPL |
| | | VRAEPILTREQLEALRAELVKTSRAKPAVSTPSLLLRVLFCAVCGEPAYKFAGGGRKHP |
| | | RYRCRSMGFPKHCGNGTVAMAEWDAFCEEQVLDLLGDAERLEKVWVAGSDSAVELAEVN |
| | | AELVDLTSLIGSPAYRAGSPQREALDARIAALAARQEELEGLEARPSGWEWRETGQRFG |
| | | DWWREQDTAAKNTWLRSMNVRLTFDVRGGLTRTIDFGDLQEYEQHLRLGSVVERLHTGM |
| | | SGSGPKKKRKV* |
| 650 | PL1948_ | ATGcctgctgctaagagagtgaaactggatTACCCCTACGACGTGCCCGACTACGCCAA |
| | DNA | GGACAAGAAATCCGATGGCAAGGACAGCCAGAAAAAAGGCAGCAGAGCCCTGGTGGTGA |
| | | TCAGACTGAGCAGAGTGACCGACGCCACCACCAGCCCCGAGAGACAGCTGGAGAGCTGC |
| | | CAGCAGCTGTGCGCCCAGAGAGGCTGGGACGTGGTGGGCGTGGCCGAGGACCTGGACGT |
| | | GAGCGGCGCCGTGGACCCCTTCGACAGAAAGAGAAGACCCAACCTGGCCAGATGGCTGG |
| | | CCTTCGAGGAGCAGCCCTTCGACGTGATCGTGGCCTACAGAGTGGACAGACTGACCAGA |
| | | AGCATCAGACACCTGCAGCAGCTGGTGCACTGGGCCGAGGACCACAAGAAGCTGGTGGT |
| | | GAGCGCCACCGAGGCCCACTTCGACACCACCACCCCCTTCGCCGCCGTGGTGATCGCCC |
| | | TGATGGGCACCGTGGCCCAGATGGAGCTGGAGGCCATCAAGGAGAGAAACAGAAGCGCC |
| | | GCCCACTTCAACATCAGAGCCGGCAAGTACAGAGGCAGCCTGCCCCCCTGGGGCTACCT |
| | | GCCCACCAGAGTGGACGGCGAGTGGAGACTGGTGCCCGACCCCGTGCAGAGAGAGAGAA |
| | | TCCTGGAGGTGTACCACAGAGTGGTGGACAACCACGAGCCCCTGCACCTGGTGGCCCAC |
| | | GACCTGAACAGAAGAGGCGTGCTGAGCCCCAAGGACTACTTCGCCCAGCTGCAGGGCAG |
| | | AGAGCCCCAGGGCAGAGAGTGGAGCGCCACCGCCCTGAAGAGAAGCATGATCAGCGAGG |
| | | CCATGCTGGGCTACGCCACCCTGAACGGCAAGACCGTGAGAGACGACGACGGCGCCCCC |
| | | CTGGTGAGAGCCGAGCCCATCCTGACCAGAGAGCAGCTGGAGGCCCTGAGAGCCGAGCT |
| | | GGTGAAGACCAGCAGAGCCAAGCCCGCCGTGAGCACCCCCAGCCTGCTGCTGAGAGTGC |
| | | TGTTCTGCGCCGTGTGCGGCGAGCCCGCCTACAAGTTCGCCGGCGGCGGCAGAAAGCAC |
| | | CCCAGATACAGATGCAGAAGCATGGGCTTCCCCAAGCACTGCGGCAACGGCACCGTGGC |
| | | CATGGCCGAGTGGGACGCCTTCTGCGAGGAGCAGGTGCTGGACCTGCTGGGCGACGCCG |
| | | AGAGACTGGAGAAGGTGTGGGTGGCCGGCAGCGACAGCGCCGTGGAGCTGGCCGAGGTG |
| | | AACGCCGAGCTGGTGGACCTGACCAGCCTGATCGGCAGCCCCGCCTACAGAGCCGGCAG |
| | | CCCCCAGAGAGAGGCCCTGGACGCCAGAATCGCCGCCCTGGCCGCCAGACAGGAGGAGC |
| | | TGGAGGGCCTGGAGGCCAGACCCAGCGGCTGGGAGTGGAGAGAGACCGGCCAGAGATTC |
| | | GGCGACTGGTGGAGAGAGCAGGACACCGCCGCCAAGAACACCTGGCTGAGAAGCATGAA |
| | | CGTGAGACTGACCTTCGACGTGAGAGGCGGCCTGACCAGAACCATCGACTTCGGCGACC |
| | | TGCAGGAGTACGAGCAGCACCTGAGACTGGGCAGCGTGGTGGAGAGACTGCACACCGGC |
| | | ATGAGCGGCAGCGGCCCCAAGAAGAAGAGAAAGGTGTGA |
| 651 | PL1949 | MPAAKRVKLDKDKKSDGKDSQKKGSRALVVIRLSRVTDATTSPERQLESCQQLCAQRGW |
| | _AA | DVVGVAEDLDVSGAVDPFDRKRRPNLARWLAFEEQPFDVIVAYRVDRLTRSIRHLQQLV |
| | | HWAEDHKKLVVSATEAHFDTTTPFAAVVIALMGTVAQMELEAIKERNRSAAHFNIRAGK |
| | | YRGSLPPWGYLPTRVDGEWRLVPDPVQRERILEVYHRVVDNHEPLHLVAHDLNRRGVLS |
| | | PKDYFAQLQGREPQGREWSATALKRSMISEAMLGYATLNGKTVRDDDGAPLVRAEPILT |
| | | REQLEALRAELVKTSRAKPAVSTPSLLLRVLFCAVCGEPAYKFAGGGRKHPRYRCRSMG |
| | | FPKHCGNGTVAMAEWDAFCEEQVLDLLGDAERLEKVWVAGSDSAVELAEVNAELVDLTS |
| | | LIGSPAYRAGSPQREALDARIAALAARQEELEGLEARPSGWEWRETGQRFGDWWREQDT |
| | | AAKNTWLRSMNVRLTFDVRGGLTRTIDFGDLQEYEQHLRLGSWVERLHTGMSGSGPKKK |
| | | RKV* |
| 652 | PL1949_ | ATGcctgctgctaagagagtgaaactggatAAGGACAAGAAATCCGATGGCAAGGACAG |
| | DNA | CCAGAAAAAAGGCAGCAGAGCCCTGGTGGTGATCAGACTGAGCAGAGTGACCGACGCCA |
| | | CCACCAGCCCCGAGAGACAGCTGGAGAGCTGCCAGCAGCTGTGCGCCCAGAGAGGCTGG |
| | | GACGTGGTGGGCGTGGCCGAGGACCTGGACGTGAGCGGCGCCGTGGACCCCTTCGACAG |
| | | AAAGAGAAGACCCAACCTGGCCAGATGGCTGGCCTTCGAGGAGCAGCCCTTCGACGTGA |
| | | TCGTGGCCTACAGAGTGGACAGACTGACCAGAAGCATCAGACACCTGCAGCAGCTGGTG |
| | | CACTGGGCCGAGGACCACAAGAAGCTGGTGGTGAGCGCCACCGAGGCCCACTTCGACAC |
| | | CACCACCCCCTTCGCCGCCGTGGTGATCGCCCTGATGGGCACCGTGGCCCAGATGGAGC |
| | | TGGAGGCCATCAAGGAGAGAAACAGAAGCGCCGCCCACTTCAACATCAGAGCCGGCAAG |
| | | TACAGAGGCAGCCTGCCCCCCTGGGGCTACCTGCCCACCAGAGTGGACGGCGAGTGGAG |
| | | ACTGGTGCCCGACCCCGTGCAGAGAGAGAGAATCCTGGAGGTGTACCACAGAGTGGTGG |
| | | ACAACCACGAGCCCCTGCACCTGGTGGCCCACGACCTGAACAGAAGAGGCGTGCTGAGC |
| | | CCCAAGGACTACTTCGCCCAGCTGCAGGGCAGAGAGCCCCAGGGCAGAGAGTGGAGCGC |
| | | CACCGCCCTGAAGAGAAGCATGATCAGCGAGGCCATGCTGGGCTACGCCACCCTGAACG |
| | | GCAAGACCGTGAGAGACGACGACGGCGCCCCCCTGGTGAGAGCCGAGCCCATCCTGACC |
| | | AGAGAGCAGCTGGAGGCCCTGAGAGCCGAGCTGGTGAAGACCAGCAGAGCCAAGCCCGC |
| | | CGTGAGCACCCCCAGCCTGCTGCTGAGAGTGCTGTTCTGCGCCGTGTGCGGCGAGCCCG |
| | | CCTACAAGTTCGCCGGCGGCGGCAGAAAGCACCCCAGATACAGATGCAGAAGCATGGGC |
| | | TTCCCCAAGCACTGCGGCAACGGCACCGTGGCCATGGCCGAGTGGGACGCCTTCTGCGA |
| | | GGAGCAGGTGCTGGACCTGCTGGGCGACGCCGAGAGACTGGAGAAGGTGTGGGTGGCCG |
| | | GCAGCGACAGCGCCGTGGAGCTGGCCGAGGTGAACGCCGAGCTGGTGGACCTGACCAGC |
| | | CTGATCGGCAGCCCCGCCTACAGAGCCGGCAGCCCCCAGAGAGAGGCCCTGGACGCCAG |
| | | AATCGCCGCCCTGGCCGCCAGACAGGAGGAGCTGGAGGGCCTGGAGGCCAGACCCAGCG |
| | | GCTGGGAGTGGAGAGAGACCGGCCAGAGATTCGGCGACTGGTGGAGAGAGCAGGACACC |
| | | GCCGCCAAGAACACCTGGCTGAGAAGCATGAACGTGAGACTGACCTTCGACGTGAGAGG |
| | | CGGCCTGACCAGAACCATCGACTTCGGCGACCTGCAGGAGTACGAGCAGCACCTGAGAC |
| | | TGGGCAGCGTGGTGGAGAGACTGCACACCGGCATGAGCGGCAGCGGCCCCAAGAAGAAG |
| | | AGAAAGGTGTGA |
| 653 | PL1950 | MPAAKRVKLDKDKKSDGKDSQKKGSRALVVIRLSRVTDATTSPERQLESCQQLCAQRGW |
| | _AA | DVVGVAEDLDVSGAVDPFDRKRRPNLARWLAFEEQPFDVIVAYRVDRLTRSIRHLQQLV |
| | | HWAEDHKKLVVSATEAHFDTTTPFAAVVIALMGTVAQMELEAIKERNRSAAHFNIRAGK |
| | | YRGSLPPWGYLPTRVDGEWRLVPDPVQRERILEVYHRVVDNHEPLHLVAHDLNRRGVLS |
| | | PKDYFAQLQGREPQGREWSATALKRSMISEAMLGYATLNGKTVRDDDGAPLVRAEPILT |
| | | REQLEALRAELVKTSRAKPAVSTPSLLLRVLFCAVCGEPAYKFAGGGRKHPRYRCRSMG |
| | | FPKHCGNGTVAMAEWDAFCEEQVLDLLGDAERLEKVWVAGSDSAVELAEVNAELVDLTS |
| | | LIGSPAYRAGSPQREALDARIAALAARQEELEGLEARPSGWEWRETGQRFGDWWREQDT |
| | | AAKNTWLRSMNVRLTFDVRGGLTRTIDFGDLQEYEQHLRLGSVVERLHTGMS* |
| 654 | PL1950_ | ATGcctgctgctaagagagtgaaactggatAAGGACAAGAAATCCGATGGCAAGGACAG |
| | DNA | CCAGAAAAAAGGCAGCAGAGCCCTGGTGGTGATCAGACTGAGCAGAGTGACCGACGCCA |
| | | CCACCAGCCCCGAGAGACAGCTGGAGAGCTGCCAGCAGCTGTGCGCCCAGAGAGGCTGG |
| | | GACGTGGTGGGCGTGGCCGAGGACCTGGACGTGAGCGGCGCCGTGGACCCCTTCGACAG |
| | | AAAGAGAAGACCCAACCTGGCCAGATGGCTGGCCTTCGAGGAGCAGCCCTTCGACGTGA |
| | | TCGTGGCCTACAGAGTGGACAGACTGACCAGAAGCATCAGACACCTGCAGCAGCTGGTG |
| | | CACTGGGCCGAGGACCACAAGAAGCTGGTGGTGAGCGCCACCGAGGCCCACTTCGACAC |
| | | CACCACCCCCTTCGCCGCCGTGGTGATCGCCCTGATGGGCACCGTGGCCCAGATGGAGC |
| | | TGGAGGCCATCAAGGAGAGAAACAGAAGCGCCGCCCACTTCAACATCAGAGCCGGCAAG |
| | | TACAGAGGCAGCCTGCCCCCCTGGGGCTACCTGCCCACCAGAGTGGACGGCGAGTGGAG |
| | | ACTGGTGCCCGACCCCGTGCAGAGAGAGAGAATCCTGGAGGTGTACCACAGAGTGGTGG |
| | | ACAACCACGAGCCCCTGCACCTGGTGGCCCACGACCTGAACAGAAGAGGCGTGCTGAGC |
| | | CCCAAGGACTACTTCGCCCAGCTGCAGGGCAGAGAGCCCCAGGGCAGAGAGTGGAGCGC |
| | | CACCGCCCTGAAGAGAAGCATGATCAGCGAGGCCATGCTGGGCTACGCCACCCTGAACG |
| | | GCAAGACCGTGAGAGACGACGACGGCGCCCCCCTGGTGAGAGCCGAGCCCATCCTGACC |
| | | AGAGAGCAGCTGGAGGCCCTGAGAGCCGAGCTGGTGAAGACCAGCAGAGCCAAGCCCGC |
| | | CGTGAGCACCCCCAGCCTGCTGCTGAGAGTGCTGTTCTGCGCCGTGTGCGGCGAGCCCG |
| | | CCTACAAGTTCGCCGGCGGCGGCAGAAAGCACCCCAGATACAGATGCAGAAGCATGGGC |
| | | TTCCCCAAGCACTGCGGCAACGGCACCGTGGCCATGGCCGAGTGGGACGCCTTCTGCGA |
| | | GGAGCAGGTGCTGGACCTGCTGGGCGACGCCGAGAGACTGGAGAAGGTGTGGGTGGCCG |
| | | GCAGCGACAGCGCCGTGGAGCTGGCCGAGGTGAACGCCGAGCTGGTGGACCTGACCAGC |
| | | CTGATCGGCAGCCCCGCCTACAGAGCCGGCAGCCCCCAGAGAGAGGCCCTGGACGCCAG |
| | | AATCGCCGCCCTGGCCGCCAGACAGGAGGAGCTGGAGGGCCTGGAGGCCAGACCCAGCG |
| | | GCTGGGAGTGGAGAGAGACCGGCCAGAGATTCGGCGACTGGTGGAGAGAGCAGGACACC |
| | | GCCGCCAAGAACACCTGGCTGAGAAGCATGAACGTGAGACTGACCTTCGACGTGAGAGG |
| | | CGGCCTGACCAGAACCATCGACTTCGGCGACCTGCAGGAGTACGAGCAGCACCTGAGAC |
| | | TGGGCAGCGTGGTGGAGAGACTGCACACCGGCATGAGCTGA |
| 655 | PL1951 | MPAAKRVKLDGSRALVVIRLSRVTDATTSPERQLESCQQLCAQRGWDVVGVAEDLDVSG |
| | _AA | AVDPFDRKRRPNLARWLAFEEQPFDVIVAYRVDRLTRSIRHLQQLVHWAEDHKKLVVSA |
| | | TEAHFDTTTPFAAVVIALMGTVAQMELEAIKERNRSAAHFNIRAGKYRGSLPPWGYLPT |
| | | RVDGEWRLVPDPVQRERILEVYHRVVDNHEPLHLVAHDLNRRGVLSPKDYFAQLQGREP |
| | | QGREWSATALKRSMISEAMLGYATLNGKTVRDDDGAPLVRAEPILTREQLEALRAELVK |
| | | TSRAKPAVSTPSLLLRVLFCAVCGEPAYKFAGGGRKHPRYRCRSMGFPKHCGNGTVAMA |
| | | EWDAFCEEQVLDLLGDAERLEKVWVAGSDSAVELAEVNAELVDLTSLIGSPAYRAGSPQ |
| | | REALDARIAALAARQEELEGLEARPSGWEWRETGQRFGDWWREQDTAAKNTWLRSMNVR |
| | | LTFDVRGGLTRTIDFGDLQEYEQHLRLGSVVERLHTGMS* |
| 656 | PL1951_ | ATGcctgctgctaagagagtgaaactggatGGCAGCAGAGCCCTGGTGGTGATCAGACT |
| | DNA | GAGCAGAGTGACCGACGCCACCACCAGCCCCGAGAGACAGCTGGAGAGCTGCCAGCAGC |
| | | TGTGCGCCCAGAGAGGCTGGGACGTGGTGGGCGTGGCCGAGGACCTGGACGTGAGCGGC |
| | | GCCGTGGACCCCTTCGACAGAAAGAGAAGACCCAACCTGGCCAGATGGCTGGCCTTCGA |
| | | GGAGCAGCCCTTCGACGTGATCGTGGCCTACAGAGTGGACAGACTGACCAGAAGCATCA |
| | | GACACCTGCAGCAGCTGGTGCACTGGGCCGAGGACCACAAGAAGCTGGTGGTGAGCGCC |
| | | ACCGAGGCCCACTTCGACACCACCACCCCCTTCGCCGCCGTGGTGATCGCCCTGATGGG |
| | | CACCGTGGCCCAGATGGAGCTGGAGGCCATCAAGGAGAGAAACAGAAGCGCCGCCCACT |
| | | TCAACATCAGAGCCGGCAAGTACAGAGGCAGCCTGCCCCCCTGGGGCTACCTGCCCACC |
| | | AGAGTGGACGGCGAGTGGAGACTGGTGCCCGACCCCGTGCAGAGAGAGAGAATCCTGGA |
| | | GGTGTACCACAGAGTGGTGGACAACCACGAGCCCCTGCACCTGGTGGCCCACGACCTGA |
| | | ACAGAAGAGGCGTGCTGAGCCCCAAGGACTACTTCGCCCAGCTGCAGGGCAGAGAGCCC |
| | | CAGGGCAGAGAGTGGAGCGCCACCGCCCTGAAGAGAAGCATGATCAGCGAGGCCATGCT |
| | | GGGCTACGCCACCCTGAACGGCAAGACCGTGAGAGACGACGACGGCGCCCCCCTGGTGA |
| | | GAGCCGAGCCCATCCTGACCAGAGAGCAGCTGGAGGCCCTGAGAGCCGAGCTGGTGAAG |
| | | ACCAGCAGAGCCAAGCCCGCCGTGAGCACCCCCAGCCTGCTGCTGAGAGTGCTGTTCTG |
| | | CGCCGTGTGCGGCGAGCCCGCCTACAAGTTCGCCGGCGGCGGCAGAAAGCACCCCAGAT |
| | | ACAGATGCAGAAGCATGGGCTTCCCCAAGCACTGCGGCAACGGCACCGTGGCCATGGCC |
| | | GAGTGGGACGCCTTCTGCGAGGAGCAGGTGCTGGACCTGCTGGGCGACGCCGAGAGACT |
| | | GGAGAAGGTGTGGGTGGCCGGCAGCGACAGCGCCGTGGAGCTGGCCGAGGTGAACGCCG |
| | | AGCTGGTGGACCTGACCAGCCTGATCGGCAGCCCCGCCTACAGAGCCGGCAGCCCCCAG |
| | | AGAGAGGCCCTGGACGCCAGAATCGCCGCCCTGGCCGCCAGACAGGAGGAGCTGGAGGG |
| | | CCTGGAGGCCAGACCCAGCGGCTGGGAGTGGAGAGAGACCGGCCAGAGATTCGGCGACT |
| | | GGTGGAGAGAGCAGGACACCGCCGCCAAGAACACCTGGCTGAGAAGCATGAACGTGAGA |
| | | CTGACCTTCGACGTGAGAGGCGGCCTGACCAGAACCATCGACTTCGGCGACCTGCAGGA |
| | | GTACGAGCAGCACCTGAGACTGGGCAGCGTGGTGGAGAGACTGCACACCGGCATGAGCT |
| | | GA |
| 657 | PL2087 | MPKKKRKVYPYDVPDYAKDGKKDKKEEDKKGSRALVVIRLSRVTDATTSPERQLESCQQ |
| | _AA | LCAQRGWDVVGVAEDLDVSGAVDPFDRKRRPNLARWLAFEEQPFDVIVAYRVDRLTRSI |
| | | RHLQQLVHWAEDHKKLVVSATEAHFDTTTPFAAVVIALMGTVAQMELEAIKERNRSAAH |
| | | FNIRAGKYRGSLPPWGYLPTRVDGEWRLVPDPVQRERILEVYHRVVDNHEPLHLVAHDL |
| | | NRRGVLSPKDYFAQLQGREPQGREWSATALKRSMISEAMLGYATLNGKTVRDDDGAPLV |
| | | RAEPILTREQLEALRAELVKTSRAKPAVSTPSLLLRVLFCAVCGEPAYKFAGGGRKHPR |
| | | YRCRSMGFPKHCGNGTVAMAEWDAFCEEQVLDLLGDAERLEKVWVAGSDSAVELAEVNA |
| | | ELVDLTSLIGSPAYRAGSPQREALDARIAALAARQEELEGLEARPSGWEWRETGQRFGD |
| | | WWREQDTAAKNTWLRSMNVRLTFDVRGGLTRTIDFGDLQEYEQHLRLGSVVERLHTGMS |
| | | * |
| 658 | PL2087_ | ATGCCCAAGAAGAAGAGAAAGGTGTACCCCTACGACGTGCCCGACTACGCCAAGGACGG |
| | DNA | CAAGAAGGACAAGAAGGAGGAGGACAAGAAGGGCAGCAGAGCCCTGGTGGTGATCAGAC |
| | | TGAGCAGAGTGACCGACGCCACCACCAGCCCCGAGAGACAGCTGGAGAGCTGCCAGCAG |
| | | CTGTGCGCCCAGAGAGGCTGGGACGTGGTGGGCGTGGCCGAGGACCTGGACGTGAGCGG |
| | | CGCCGTGGACCCCTTCGACAGAAAGAGAAGACCCAACCTGGCCAGATGGCTGGCCTTCG |
| | | AGGAGCAGCCCTTCGACGTGATCGTGGCCTACAGAGTGGACAGACTGACCAGAAGCATC |
| | | AGACACCTGCAGCAGCTGGTGCACTGGGCCGAGGACCACAAGAAGCTGGTGGTGAGCGC |
| | | CACCGAGGCCCACTTCGACACCACCACCCCCTTCGCCGCCGTGGTGATCGCCCTGATGG |
| | | GCACCGTGGCCCAGATGGAGCTGGAGGCCATCAAGGAGAGAAACAGAAGCGCCGCCCAC |
| | | TTCAACATCAGAGCCGGCAAGTACAGAGGCAGCCTGCCCCCCTGGGGCTACCTGCCCAC |
| | | CAGAGTGGACGGCGAGTGGAGACTGGTGCCCGACCCCGTGCAGAGAGAGAGAATCCTGG |
| | | AGGTGTACCACAGAGTGGTGGACAACCACGAGCCCCTGCACCTGGTGGCCCACGACCTG |
| | | AACAGAAGAGGCGTGCTGAGCCCCAAGGACTACTTCGCCCAGCTGCAGGGCAGAGAGCC |
| | | CCAGGGCAGAGAGTGGAGCGCCACCGCCCTGAAGAGAAGCATGATCAGCGAGGCCATGC |
| | | TGGGCTACGCCACCCTGAACGGCAAGACCGTGAGAGACGACGACGGCGCCCCCCTGGTG |
| | | AGAGCCGAGCCCATCCTGACCAGAGAGCAGCTGGAGGCCCTGAGAGCCGAGCTGGTGAA |
| | | GACCAGCAGAGCCAAGCCCGCCGTGAGCACCCCCAGCCTGCTGCTGAGAGTGCTGTTCT |
| | | GCGCCGTGTGCGGCGAGCCCGCCTACAAGTTCGCCGGCGGCGGCAGAAAGCACCCCAGA |
| | | TACAGATGCAGAAGCATGGGCTTCCCCAAGCACTGCGGCAACGGCACCGTGGCCATGGC |
| | | CGAGTGGGACGCCTTCTGCGAGGAGCAGGTGCTGGACCTGCTGGGCGACGCCGAGAGAC |
| | | TGGAGAAGGTGTGGGTGGCCGGCAGCGACAGCGCCGTGGAGCTGGCCGAGGTGAACGCC |
| | | GAGCTGGTGGACCTGACCAGCCTGATCGGCAGCCCCGCCTACAGAGCCGGCAGCCCCCA |
| | | GAGAGAGGCCCTGGACGCCAGAATCGCCGCCCTGGCCGCCAGACAGGAGGAGCTGGAGG |
| | | GCCTGGAGGCCAGACCCAGCGGCTGGGAGTGGAGAGAGACCGGCCAGAGATTCGGCGAC |
| | | TGGTGGAGAGAGCAGGACACCGCCGCCAAGAACACCTGGCTGAGAAGCATGAACGTGAG |
| | | ACTGACCTTCGACGTGAGAGGCGGCCTGACCAGAACCATCGACTTCGGCGACCTGCAGG |
| | | AGTACGAGCAGCACCTGAGACTGGGCAGCGTGGTGGAGAGACTGCACACCGGCATGAGC |
| | | TGA |
| 659 | PL2088 | MPKKKRKVYPYDVPDYAKDGKKDKKEEDKKGSRALVVIRLSRVTDATTSPERQLESCQQ |
| | _AA | LCAQRGWDVVGVAEDLDVSGAVDPFDRKRRPNLARWLAFEEQPFDVIVAYRVDRLTRSI |
| | | RHLQQLVHWAEDHKKLVVSATEAHFDTTTPFAAVVIALMGTVAQMELEAIKERNRSAAH |
| | | FNIRAGKYRGSLPPWGYLPTRVDGEWRLVPDPVQRERILEVYHRVVDNHEPLHLVAHDL |
| | | NRRGVLSPKDYFAQLQGREPQGREWSATALKRSMISEAMLGYATLNGKTVRDDDGAPLV |
| | | RAEPILTREQLEALRAELVKTSRAKPAVSTPSLLLRVLFCAVCGEPAYKFAGGGRKHPR |
| | | YRCRSMGFPKHCGNGTVAMAEWDAFCEEQVLDLLGDAERLEKVWVAGSDSAVELAEVNA |
| | | ELVDLTSLIGSPAYRAGSPQREALDARIAALAARQEELEGLEARPSGWEWRETGQRFGD |
| | | WWREQDTAAKNTWLRSMNVRLTFDVRGGLTRTIDFGDLQEYEQHLRLGSVVERLHTGMS |
| | | GSGKDGKKDKKEEDKK* |
| 660 | PL2088_ | ATGCCCAAGAAGAAGAGAAAGGTGTACCCCTACGACGTGCCCGACTACGCCAAGGACGG |
| | DNA | CAAGAAGGACAAGAAGGAGGAGGACAAGAAGGGCAGCAGAGCCCTGGTGGTGATCAGAC |
| | | TGAGCAGAGTGACCGACGCCACCACCAGCCCCGAGAGACAGCTGGAGAGCTGCCAGCAG |
| | | CTGTGCGCCCAGAGAGGCTGGGACGTGGTGGGCGTGGCCGAGGACCTGGACGTGAGCGG |
| | | CGCCGTGGACCCCTTCGACAGAAAGAGAAGACCCAACCTGGCCAGATGGCTGGCCTTCG |
| | | AGGAGCAGCCCTTCGACGTGATCGTGGCCTACAGAGTGGACAGACTGACCAGAAGCATC |
| | | AGACACCTGCAGCAGCTGGTGCACTGGGCCGAGGACCACAAGAAGCTGGTGGTGAGCGC |
| | | CACCGAGGCCCACTTCGACACCACCACCCCCTTCGCCGCCGTGGTGATCGCCCTGATGG |
| | | GCACCGTGGCCCAGATGGAGCTGGAGGCCATCAAGGAGAGAAACAGAAGCGCCGCCCAC |
| | | TTCAACATCAGAGCCGGCAAGTACAGAGGCAGCCTGCCCCCCTGGGGCTACCTGCCCAC |
| | | CAGAGTGGACGGCGAGTGGAGACTGGTGCCCGACCCCGTGCAGAGAGAGAGAATCCTGG |
| | | AGGTGTACCACAGAGTGGTGGACAACCACGAGCCCCTGCACCTGGTGGCCCACGACCTG |
| | | AACAGAAGAGGCGTGCTGAGCCCCAAGGACTACTTCGCCCAGCTGCAGGGCAGAGAGCC |
| | | CCAGGGCAGAGAGTGGAGCGCCACCGCCCTGAAGAGAAGCATGATCAGCGAGGCCATGC |
| | | TGGGCTACGCCACCCTGAACGGCAAGACCGTGAGAGACGACGACGGCGCCCCCCTGGTG |
| | | AGAGCCGAGCCCATCCTGACCAGAGAGCAGCTGGAGGCCCTGAGAGCCGAGCTGGTGAA |
| | | GACCAGCAGAGCCAAGCCCGCCGTGAGCACCCCCAGCCTGCTGCTGAGAGTGCTGTTCT |
| | | GCGCCGTGTGCGGCGAGCCCGCCTACAAGTTCGCCGGCGGCGGCAGAAAGCACCCCAGA |
| | | TACAGATGCAGAAGCATGGGCTTCCCCAAGCACTGCGGCAACGGCACCGTGGCCATGGC |
| | | CGAGTGGGACGCCTTCTGCGAGGAGCAGGTGCTGGACCTGCTGGGCGACGCCGAGAGAC |
| | | TGGAGAAGGTGTGGGTGGCCGGCAGCGACAGCGCCGTGGAGCTGGCCGAGGTGAACGCC |
| | | GAGCTGGTGGACCTGACCAGCCTGATCGGCAGCCCCGCCTACAGAGCCGGCAGCCCCCA |
| | | GAGAGAGGCCCTGGACGCCAGAATCGCCGCCCTGGCCGCCAGACAGGAGGAGCTGGAGG |
| | | GCCTGGAGGCCAGACCCAGCGGCTGGGAGTGGAGAGAGACCGGCCAGAGATTCGGCGAC |
| | | TGGTGGAGAGAGCAGGACACCGCCGCCAAGAACACCTGGCTGAGAAGCATGAACGTGAG |
| | | ACTGACCTTCGACGTGAGAGGCGGCCTGACCAGAACCATCGACTTCGGCGACCTGCAGG |
| | | AGTACGAGCAGCACCTGAGACTGGGCAGCGTGGTGGAGAGACTGCACACCGGCATGAGC |
| | | GGCAGCGGCAAGGACGGCAAGAAGGACAAGAAGGAGGAGGACAAGAAGTGA |
| 661 | PL2089 | MPKKKRKVYPYDVPDYAQPRFAAAGSGKDGKKDKKEEDKKGSRALVVIRLSRVTDATTS |
| | _AA | PERQLESCQQLCAQRGWDVVGVAEDLDVSGAVDPFDRKRRPNLARWLAFEEQPFDVIVA |
| | | YRVDRLTRSIRHLQQLVHWAEDHKKLVVSATEAHFDTTTPFAAVVIALMGTVAQMELEA |
| | | IKERNRSAAHFNIRAGKYRGSLPPWGYLPTRVDGEWRLVPDPVQRERILEVYHRVVDNH |
| | | EPLHLVAHDLNRRGVLSPKDYFAQLQGREPQGREWSATALKRSMISEAMLGYATLNGKT |
| | | VRDDDGAPLVRAEPILTREQLEALRAELVKTSRAKPAVSTPSLLLRVLFCAVCGEPAYK |
| | | FAGGGRKHPRYRCRSMGFPKHCGNGTVAMAEWDAFCEEQVLDLLGDAERLEKVWVAGSD |
| | | SAVELAEVNAELVDLTSLIGSPAYRAGSPQREALDARIAALAARQEELEGLEARPSGWE |
| | | WRETGQRFGDWWREQDTAAKNTWLRSMNVRLTFDVRGGLTRTIDFGDLQEYEQHLRLGS |
| | | WVERLHTGMS* |
| 662 | PL2089_ | ATGCCCAAGAAGAAGAGAAAGGTGTACCCCTACGACGTGCCCGACTACGCCCAACCGCG |
| | DNA | GTTCGCGGCCGCTGGCAGCGGCAAGGACGGCAAGAAGGACAAGAAGGAGGAGGACAAGA |
| | | AGGGCAGCAGAGCCCTGGTGGTGATCAGACTGAGCAGAGTGACCGACGCCACCACCAGC |
| | | CCCGAGAGACAGCTGGAGAGCTGCCAGCAGCTGTGCGCCCAGAGAGGCTGGGACGTGGT |
| | | GGGCGTGGCCGAGGACCTGGACGTGAGCGGCGCCGTGGACCCCTTCGACAGAAAGAGAA |
| | | GACCCAACCTGGCCAGATGGCTGGCCTTCGAGGAGCAGCCCTTCGACGTGATCGTGGCC |
| | | TACAGAGTGGACAGACTGACCAGAAGCATCAGACACCTGCAGCAGCTGGTGCACTGGGC |
| | | CGAGGACCACAAGAAGCTGGTGGTGAGCGCCACCGAGGCCCACTTCGACACCACCACCC |
| | | CCTTCGCCGCCGTGGTGATCGCCCTGATGGGCACCGTGGCCCAGATGGAGCTGGAGGCC |
| | | ATCAAGGAGAGAAACAGAAGCGCCGCCCACTTCAACATCAGAGCCGGCAAGTACAGAGG |
| | | CAGCCTGCCCCCCTGGGGCTACCTGCCCACCAGAGTGGACGGCGAGTGGAGACTGGTGC |
| | | CCGACCCCGTGCAGAGAGAGAGAATCCTGGAGGTGTACCACAGAGTGGTGGACAACCAC |
| | | GAGCCCCTGCACCTGGTGGCCCACGACCTGAACAGAAGAGGCGTGCTGAGCCCCAAGGA |
| | | CTACTTCGCCCAGCTGCAGGGCAGAGAGCCCCAGGGCAGAGAGTGGAGCGCCACCGCCC |
| | | TGAAGAGAAGCATGATCAGCGAGGCCATGCTGGGCTACGCCACCCTGAACGGCAAGACC |
| | | GTGAGAGACGACGACGGCGCCCCCCTGGTGAGAGCCGAGCCCATCCTGACCAGAGAGCA |
| | | GCTGGAGGCCCTGAGAGCCGAGCTGGTGAAGACCAGCAGAGCCAAGCCCGCCGTGAGCA |
| | | CCCCCAGCCTGCTGCTGAGAGTGCTGTTCTGCGCCGTGTGCGGCGAGCCCGCCTACAAG |
| | | TTCGCCGGCGGCGGCAGAAAGCACCCCAGATACAGATGCAGAAGCATGGGCTTCCCCAA |
| | | GCACTGCGGCAACGGCACCGTGGCCATGGCCGAGTGGGACGCCTTCTGCGAGGAGCAGG |
| | | TGCTGGACCTGCTGGGCGACGCCGAGAGACTGGAGAAGGTGTGGGTGGCCGGCAGCGAC |
| | | AGCGCCGTGGAGCTGGCCGAGGTGAACGCCGAGCTGGTGGACCTGACCAGCCTGATCGG |
| | | CAGCCCCGCCTACAGAGCCGGCAGCCCCCAGAGAGAGGCCCTGGACGCCAGAATCGCCG |
| | | CCCTGGCCGCCAGACAGGAGGAGCTGGAGGGCCTGGAGGCCAGACCCAGCGGCTGGGAG |
| | | TGGAGAGAGACCGGCCAGAGATTCGGCGACTGGTGGAGAGAGCAGGACACCGCCGCCAA |
| | | GAACACCTGGCTGAGAAGCATGAACGTGAGACTGACCTTCGACGTGAGAGGCGGCCTGA |
| | | CCAGAACCATCGACTTCGGCGACCTGCAGGAGTACGAGCAGCACCTGAGACTGGGCAGC |
| | | GTGGTGGAGAGACTGCACACCGGCATGAGCTGA |
| 663 | PL2090 | MPKKKRKVYPYDVPDYAQPRFAAAGSGKDGKKDKKEEDKKGSRALVVIRLSRVTDATTS |
| | _AA | PERQLESCQQLCAQRGWDVVGVAEDLDVSGAVDPFDRKRRPNLARWLAFEEQPFDVIVA |
| | | YRVDRLTRSIRHLQQLVHWAEDHKKLVVSATEAHFDTTTPFAAVVIALMGTVAQMELEA |
| | | IKERNRSAAHFNIRAGKYRGSLPPWGYLPTRVDGEWRLVPDPVQRERILEVYHRVVDNH |
| | | EPLHLVAHDLNRRGVLSPKDYFAQLQGREPQGREWSATALKRSMISEAMLGYATLNGKT |
| | | VRDDDGAPLVRAEPILTREQLEALRAELVKTSRAKPAVSTPSLLLRVLFCAVCGEPAYK |
| | | FAGGGRKHPRYRCRSMGFPKHCGNGTVAMAEWDAFCEEQVLDLLGDAERLEKVWVAGSD |
| | | SAVELAEVNAELVDLTSLIGSPAYRAGSPQREALDARIAALAARQEELEGLEARPSGWE |
| | | WRETGQRFGDWWREQDTAAKNTWLRSMNVRLTFDVRGGLTRTIDFGDLQEYEQHLRLGS |
| | | WVERLHTGMSGSGKDGKKDKKEEDKK* |
| 664 | PL2090_ | ATGCCCAAGAAGAAGAGAAAGGTGTACCCCTACGACGTGCCCGACTACGCCCAACCGCG |
| | DNA | GTTCGCGGCCGCTGGCAGCGGCAAGGACGGCAAGAAGGACAAGAAGGAGGAGGACAAGA |
| | | AGGGCAGCAGAGCCCTGGTGGTGATCAGACTGAGCAGAGTGACCGACGCCACCACCAGC |
| | | CCCGAGAGACAGCTGGAGAGCTGCCAGCAGCTGTGCGCCCAGAGAGGCTGGGACGTGGT |
| | | GGGCGTGGCCGAGGACCTGGACGTGAGCGGCGCCGTGGACCCCTTCGACAGAAAGAGAA |
| | | GACCCAACCTGGCCAGATGGCTGGCCTTCGAGGAGCAGCCCTTCGACGTGATCGTGGCC |
| | | TACAGAGTGGACAGACTGACCAGAAGCATCAGACACCTGCAGCAGCTGGTGCACTGGGC |
| | | CGAGGACCACAAGAAGCTGGTGGTGAGCGCCACCGAGGCCCACTTCGACACCACCACCC |
| | | CCTTCGCCGCCGTGGTGATCGCCCTGATGGGCACCGTGGCCCAGATGGAGCTGGAGGCC |
| | | ATCAAGGAGAGAAACAGAAGCGCCGCCCACTTCAACATCAGAGCCGGCAAGTACAGAGG |
| | | CAGCCTGCCCCCCTGGGGCTACCTGCCCACCAGAGTGGACGGCGAGTGGAGACTGGTGC |
| | | CCGACCCCGTGCAGAGAGAGAGAATCCTGGAGGTGTACCACAGAGTGGTGGACAACCAC |
| | | GAGCCCCTGCACCTGGTGGCCCACGACCTGAACAGAAGAGGCGTGCTGAGCCCCAAGGA |
| | | CTACTTCGCCCAGCTGCAGGGCAGAGAGCCCCAGGGCAGAGAGTGGAGCGCCACCGCCC |
| | | TGAAGAGAAGCATGATCAGCGAGGCCATGCTGGGCTACGCCACCCTGAACGGCAAGACC |
| | | GTGAGAGACGACGACGGCGCCCCCCTGGTGAGAGCCGAGCCCATCCTGACCAGAGAGCA |
| | | GCTGGAGGCCCTGAGAGCCGAGCTGGTGAAGACCAGCAGAGCCAAGCCCGCCGTGAGCA |
| | | CCCCCAGCCTGCTGCTGAGAGTGCTGTTCTGCGCCGTGTGCGGCGAGCCCGCCTACAAG |
| | | TTCGCCGGCGGCGGCAGAAAGCACCCCAGATACAGATGCAGAAGCATGGGCTTCCCCAA |
| | | GCACTGCGGCAACGGCACCGTGGCCATGGCCGAGTGGGACGCCTTCTGCGAGGAGCAGG |
| | | TGCTGGACCTGCTGGGCGACGCCGAGAGACTGGAGAAGGTGTGGGTGGCCGGCAGCGAC |
| | | AGCGCCGTGGAGCTGGCCGAGGTGAACGCCGAGCTGGTGGACCTGACCAGCCTGATCGG |
| | | CAGCCCCGCCTACAGAGCCGGCAGCCCCCAGAGAGAGGCCCTGGACGCCAGAATCGCCG |
| | | CCCTGGCCGCCAGACAGGAGGAGCTGGAGGGCCTGGAGGCCAGACCCAGCGGCTGGGAG |
| | | TGGAGAGAGACCGGCCAGAGATTCGGCGACTGGTGGAGAGAGCAGGACACCGCCGCCAA |
| | | GAACACCTGGCTGAGAAGCATGAACGTGAGACTGACCTTCGACGTGAGAGGCGGCCTGA |
| | | CCAGAACCATCGACTTCGGCGACCTGCAGGAGTACGAGCAGCACCTGAGACTGGGCAGC |
| | | GTGGTGGAGAGACTGCACACCGGCATGAGCGGCAGCGGCAAGGACGGCAAGAAGGACAA |
| | | GAAGGAGGAGGACAAGAAGTGA |
| 665 | PL2091 | MPKKKRKVYPYDVPDYAKDGKKDKKEEDKKGSGQPRFAAAGSRALVVIRLSRVTDATTS |
| | _AA | PERQLESCQQLCAQRGWDVVGVAEDLDVSGAVDPFDRKRRPNLARWLAFEEQPFDVIVA |
| | | YRVDRLTRSIRHLQQLVHWAEDHKKLVVSATEAHFDTTTPFAAVVIALMGTVAQMELEA |
| | | IKERNRSAAHFNIRAGKYRGSLPPWGYLPTRVDGEWRLVPDPVQRERILEVYHRVVDNH |
| | | EPLHLVAHDLNRRGVLSPKDYFAQLQGREPQGREWSATALKRSMISEAMLGYATLNGKT |
| | | VRDDDGAPLVRAEPILTREQLEALRAELVKTSRAKPAVSTPSLLLRVLFCAVCGEPAYK |
| | | FAGGGRKHPRYRCRSMGFPKHCGNGTVAMAEWDAFCEEQVLDLLGDAERLEKVWVAGSD |
| | | SAVELAEVNAELVDLTSLIGSPAYRAGSPQREALDARIAALAARQEELEGLEARPSGWE |
| | | WRETGQRFGDWWREQDTAAKNTWLRSMNVRLTFDVRGGLTRTIDFGDLQEYEQHLRLGS |
| | | VVERLHTGMS* |
| 666 | PL2091_ | ATGCCCAAGAAGAAGAGAAAGGTGTACCCCTACGACGTGCCCGACTACGCCAAGGACGG |
| | DNA | CAAGAAGGACAAGAAGGAGGAGGACAAGAAGGGCAGCGGCCAACCGCGGTTCGCGGCCG |
| | | CTGGCAGCAGAGCCCTGGTGGTGATCAGACTGAGCAGAGTGACCGACGCCACCACCAGC |
| | | CCCGAGAGACAGCTGGAGAGCTGCCAGCAGCTGTGCGCCCAGAGAGGCTGGGACGTGGT |
| | | GGGCGTGGCCGAGGACCTGGACGTGAGCGGCGCCGTGGACCCCTTCGACAGAAAGAGAA |
| | | GACCCAACCTGGCCAGATGGCTGGCCTTCGAGGAGCAGCCCTTCGACGTGATCGTGGCC |
| | | TACAGAGTGGACAGACTGACCAGAAGCATCAGACACCTGCAGCAGCTGGTGCACTGGGC |
| | | CGAGGACCACAAGAAGCTGGTGGTGAGCGCCACCGAGGCCCACTTCGACACCACCACCC |
| | | CCTTCGCCGCCGTGGTGATCGCCCTGATGGGCACCGTGGCCCAGATGGAGCTGGAGGCC |
| | | ATCAAGGAGAGAAACAGAAGCGCCGCCCACTTCAACATCAGAGCCGGCAAGTACAGAGG |
| | | CAGCCTGCCCCCCTGGGGCTACCTGCCCACCAGAGTGGACGGCGAGTGGAGACTGGTGC |
| | | CCGACCCCGTGCAGAGAGAGAGAATCCTGGAGGTGTACCACAGAGTGGTGGACAACCAC |
| | | GAGCCCCTGCACCTGGTGGCCCACGACCTGAACAGAAGAGGCGTGCTGAGCCCCAAGGA |
| | | CTACTTCGCCCAGCTGCAGGGCAGAGAGCCCCAGGGCAGAGAGTGGAGCGCCACCGCCC |
| | | TGAAGAGAAGCATGATCAGCGAGGCCATGCTGGGCTACGCCACCCTGAACGGCAAGACC |
| | | GTGAGAGACGACGACGGCGCCCCCCTGGTGAGAGCCGAGCCCATCCTGACCAGAGAGCA |
| | | GCTGGAGGCCCTGAGAGCCGAGCTGGTGAAGACCAGCAGAGCCAAGCCCGCCGTGAGCA |
| | | CCCCCAGCCTGCTGCTGAGAGTGCTGTTCTGCGCCGTGTGCGGCGAGCCCGCCTACAAG |
| | | TTCGCCGGCGGCGGCAGAAAGCACCCCAGATACAGATGCAGAAGCATGGGCTTCCCCAA |
| | | GCACTGCGGCAACGGCACCGTGGCCATGGCCGAGTGGGACGCCTTCTGCGAGGAGCAGG |
| | | TGCTGGACCTGCTGGGCGACGCCGAGAGACTGGAGAAGGTGTGGGTGGCCGGCAGCGAC |
| | | AGCGCCGTGGAGCTGGCCGAGGTGAACGCCGAGCTGGTGGACCTGACCAGCCTGATCGG |
| | | CAGCCCCGCCTACAGAGCCGGCAGCCCCCAGAGAGAGGCCCTGGACGCCAGAATCGCCG |
| | | CCCTGGCCGCCAGACAGGAGGAGCTGGAGGGCCTGGAGGCCAGACCCAGCGGCTGGGAG |
| | | TGGAGAGAGACCGGCCAGAGATTCGGCGACTGGTGGAGAGAGCAGGACACCGCCGCCAA |
| | | GAACACCTGGCTGAGAAGCATGAACGTGAGACTGACCTTCGACGTGAGAGGCGGCCTGA |
| | | CCAGAACCATCGACTTCGGCGACCTGCAGGAGTACGAGCAGCACCTGAGACTGGGCAGC |
| | | GTGGTGGAGAGACTGCACACCGGCATGAGCTGA |
| 667 | PL2092 | MPKKKRKVYPYDVPDYAKDGKKDKKEEDKKGSGQPRFAAAGSRALWIRLSRVTDATTSP |
| | _AA | ERQLESCQQLCAQRGWDVVGVAEDLDVSGAVDPFDRKRRPNLARWLAFEEQPFDVIVAY |
| | | RVDRLTRSIRHLQQLVHWAEDHKKLVVSATEAHFDTTTPFAAVVIALMGTVAQMELEAI |
| | | KERNRSAAHFNIRAGKYRGSLPPWGYLPTRVDGEWRLVPDPVQRERILEVYHRVVDNHE |
| | | PLHLVAHDLNRRGVLSPKDYFAQLQGREPQGREWSATALKRSMISEAMLGYATLNGKTV |
| | | RDDDGAPLVRAEPILTREQLEALRAELVKTSRAKPAVSTPSLLLRVLFCAVCGEPAYKF |
| | | AGGGRKHPRYRCRSMGFPKHCGNGTVAMAEWDAFCEEQVLDLLGDAERLEKVWVAGSDS |
| | | AVELAEVNAELVDLTSLIGSPAYRAGSPQREALDARIAALAARQEELEGLEARPSGWEW |
| | | RETGQRFGDWWREQDTAAKNTWLRSMNVRLTFDVRGGLTRTIDFGDLQEYEQHLRLGSW |
| | | VERLHTGMSGSGKDGKKDKKEEDKK* |
| 668 | PL2092_ | ATGCCCAAGAAGAAGAGAAAGGTGTACCCCTACGACGTGCCCGACTACGCCAAGGACGG |
| | DNA | CAAGAAGGACAAGAAGGAGGAGGACAAGAAGGGCAGCGGCCAACCGCGGTTCGCGGCCG |
| | | CTGGCAGCAGAGCCCTGGTGGTGATCAGACTGAGCAGAGTGACCGACGCCACCACCAGC |
| | | CCCGAGAGACAGCTGGAGAGCTGCCAGCAGCTGTGCGCCCAGAGAGGCTGGGACGTGGT |
| | | GGGCGTGGCCGAGGACCTGGACGTGAGCGGCGCCGTGGACCCCTTCGACAGAAAGAGAA |
| | | GACCCAACCTGGCCAGATGGCTGGCCTTCGAGGAGCAGCCCTTCGACGTGATCGTGGCC |
| | | TACAGAGTGGACAGACTGACCAGAAGCATCAGACACCTGCAGCAGCTGGTGCACTGGGC |
| | | CGAGGACCACAAGAAGCTGGTGGTGAGCGCCACCGAGGCCCACTTCGACACCACCACCC |
| | | CCTTCGCCGCCGTGGTGATCGCCCTGATGGGCACCGTGGCCCAGATGGAGCTGGAGGCC |
| | | ATCAAGGAGAGAAACAGAAGCGCCGCCCACTTCAACATCAGAGCCGGCAAGTACAGAGG |
| | | CAGCCTGCCCCCCTGGGGCTACCTGCCCACCAGAGTGGACGGCGAGTGGAGACTGGTGC |
| | | CCGACCCCGTGCAGAGAGAGAGAATCCTGGAGGTGTACCACAGAGTGGTGGACAACCAC |
| | | GAGCCCCTGCACCTGGTGGCCCACGACCTGAACAGAAGAGGCGTGCTGAGCCCCAAGGA |
| | | CTACTTCGCCCAGCTGCAGGGCAGAGAGCCCCAGGGCAGAGAGTGGAGCGCCACCGCCC |
| | | TGAAGAGAAGCATGATCAGCGAGGCCATGCTGGGCTACGCCACCCTGAACGGCAAGACC |
| | | GTGAGAGACGACGACGGCGCCCCCCTGGTGAGAGCCGAGCCCATCCTGACCAGAGAGCA |
| | | GCTGGAGGCCCTGAGAGCCGAGCTGGTGAAGACCAGCAGAGCCAAGCCCGCCGTGAGCA |
| | | CCCCCAGCCTGCTGCTGAGAGTGCTGTTCTGCGCCGTGTGCGGCGAGCCCGCCTACAAG |
| | | TTCGCCGGCGGCGGCAGAAAGCACCCCAGATACAGATGCAGAAGCATGGGCTTCCCCAA |
| | | GCACTGCGGCAACGGCACCGTGGCCATGGCCGAGTGGGACGCCTTCTGCGAGGAGCAGG |
| | | TGCTGGACCTGCTGGGCGACGCCGAGAGACTGGAGAAGGTGTGGGTGGCCGGCAGCGAC |
| | | AGCGCCGTGGAGCTGGCCGAGGTGAACGCCGAGCTGGTGGACCTGACCAGCCTGATCGG |
| | | CAGCCCCGCCTACAGAGCCGGCAGCCCCCAGAGAGAGGCCCTGGACGCCAGAATCGCCG |
| | | CCCTGGCCGCCAGACAGGAGGAGCTGGAGGGCCTGGAGGCCAGACCCAGCGGCTGGGAG |
| | | TGGAGAGAGACCGGCCAGAGATTCGGCGACTGGTGGAGAGAGCAGGACACCGCCGCCAA |
| | | GAACACCTGGCTGAGAAGCATGAACGTGAGACTGACCTTCGACGTGAGAGGCGGCCTGA |
| | | CCAGAACCATCGACTTCGGCGACCTGCAGGAGTACGAGCAGCACCTGAGACTGGGCAGC |
| | | GTGGTGGAGAGACTGCACACCGGCATGAGCGGCAGCGGCAAGGACGGCAAGAAGGACAA |
| | | GAAGGAGGAGGACAAGAAGTGA |
| 669 | PL2093 | MPKKKRKVYPYDVPDYAKDGKKDKKEEDKKKDGKKDKKEEDKKGSRALVVIRLSRVTDA |
| | _AA | TTSPERQLESCQQLCAQRGWDVVGVAEDLDVSGAVDPFDRKRRPNLARWLAFEEQPFDV |
| | | IVAYRVDRLTRSIRHLQQLVHWAEDHKKLVVSATEAHFDTTTPFAAVVIALMGTVAQME |
| | | LEAIKERNRSAAHFNIRAGKYRGSLPPWGYLPTRVDGEWRLVPDPVQRERILEVYHRWD |
| | | NHEPLHLVAHDLNRRGVLSPKDYFAQLQGREPQGREWSATALKRSMISEAMLGYATLNG |
| | | KTVRDDDGAPLVRAEPILTREQLEALRAELVKTSRAKPAVSTPSLLLRVLFCAVCGEPA |
| | | YKFAGGGRKHPRYRCRSMGFPKHCGNGTVAMAEWDAFCEEQVLDLLGDAERLEKVWVAG |
| | | SDSAVELAEVNAELVDLTSLIGSPAYRAGSPQREALDARIAALAARQEELEGLEARPSG |
| | | WEWRETGQRFGDWWREQDTAAKNTWLRSMNVRLTFDVRGGLTRTIDFGDLQEYEQHLRL |
| | | GSVVERLHTGMS* |
| 670 | PL2093_ | ATGCCCAAGAAGAAGAGAAAGGTGTACCCCTACGACGTGCCCGACTACGCCAAGGACGG |
| | DNA | CAAGAAGGACAAGAAGGAGGAGGACAAGAAGAAGGACGGCAAGAAGGACAAGAAGGAGG |
| | | AGGACAAGAAGGGCAGCAGAGCCCTGGTGGTGATCAGACTGAGCAGAGTGACCGACGCC |
| | | ACCACCAGCCCCGAGAGACAGCTGGAGAGCTGCCAGCAGCTGTGCGCCCAGAGAGGCTG |
| | | GGACGTGGTGGGCGTGGCCGAGGACCTGGACGTGAGCGGCGCCGTGGACCCCTTCGACA |
| | | GAAAGAGAAGACCCAACCTGGCCAGATGGCTGGCCTTCGAGGAGCAGCCCTTCGACGTG |
| | | ATCGTGGCCTACAGAGTGGACAGACTGACCAGAAGCATCAGACACCTGCAGCAGCTGGT |
| | | GCACTGGGCCGAGGACCACAAGAAGCTGGTGGTGAGCGCCACCGAGGCCCACTTCGACA |
| | | CCACCACCCCCTTCGCCGCCGTGGTGATCGCCCTGATGGGCACCGTGGCCCAGATGGAG |
| | | CTGGAGGCCATCAAGGAGAGAAACAGAAGCGCCGCCCACTTCAACATCAGAGCCGGCAA |
| | | GTACAGAGGCAGCCTGCCCCCCTGGGGCTACCTGCCCACCAGAGTGGACGGCGAGTGGA |
| | | GACTGGTGCCCGACCCCGTGCAGAGAGAGAGAATCCTGGAGGTGTACCACAGAGTGGTG |
| | | GACAACCACGAGCCCCTGCACCTGGTGGCCCACGACCTGAACAGAAGAGGCGTGCTGAG |
| | | CCCCAAGGACTACTTCGCCCAGCTGCAGGGCAGAGAGCCCCAGGGCAGAGAGTGGAGCG |
| | | CCACCGCCCTGAAGAGAAGCATGATCAGCGAGGCCATGCTGGGCTACGCCACCCTGAAC |
| | | GGCAAGACCGTGAGAGACGACGACGGCGCCCCCCTGGTGAGAGCCGAGCCCATCCTGAC |
| | | CAGAGAGCAGCTGGAGGCCCTGAGAGCCGAGCTGGTGAAGACCAGCAGAGCCAAGCCCG |
| | | CCGTGAGCACCCCCAGCCTGCTGCTGAGAGTGCTGTTCTGCGCCGTGTGCGGCGAGCCC |
| | | GCCTACAAGTTCGCCGGCGGCGGCAGAAAGCACCCCAGATACAGATGCAGAAGCATGGG |
| | | CTTCCCCAAGCACTGCGGCAACGGCACCGTGGCCATGGCCGAGTGGGACGCCTTCTGCG |
| | | AGGAGCAGGTGCTGGACCTGCTGGGCGACGCCGAGAGACTGGAGAAGGTGTGGGTGGCC |
| | | GGCAGCGACAGCGCCGTGGAGCTGGCCGAGGTGAACGCCGAGCTGGTGGACCTGACCAG |
| | | CCTGATCGGCAGCCCCGCCTACAGAGCCGGCAGCCCCCAGAGAGAGGCCCTGGACGCCA |
| | | GAATCGCCGCCCTGGCCGCCAGACAGGAGGAGCTGGAGGGCCTGGAGGCCAGACCCAGC |
| | | GGCTGGGAGTGGAGAGAGACCGGCCAGAGATTCGGCGACTGGTGGAGAGAGCAGGACAC |
| | | CGCCGCCAAGAACACCTGGCTGAGAAGCATGAACGTGAGACTGACCTTCGACGTGAGAG |
| | | GCGGCCTGACCAGAACCATCGACTTCGGCGACCTGCAGGAGTACGAGCAGCACCTGAGA |
| | | CTGGGCAGCGTGGTGGAGAGACTGCACACCGGCATGAGCTGA |
| 671 | PL2094 | MPKKKRKVYPYDVPDYAKDGKKDKKEEDKKKDGKKDKKEEDKKGSRALVVIRLSRVTDA |
| | _AA | TTSPERQLESCQQLCAQRGWDVVGVAEDLDVSGAVDPFDRKRRPNLARWLAFEEQPFDV |
| | | IVAYRVDRLTRSIRHLQQLVHWAEDHKKLVVSATEAHFDTTTPFAAVVIALMGTVAQME |
| | | LEAIKERNRSAAHFNIRAGKYRGSLPPWGYLPTRVDGEWRLVPDPVQRERILEVYHRWD |
| | | NHEPLHLVAHDLNRRGVLSPKDYFAQLQGREPQGREWSATALKRSMISEAMLGYATLNG |
| | | KTVRDDDGAPLVRAEPILTREQLEALRAELVKTSRAKPAVSTPSLLLRVLFCAVCGEPA |
| | | YKFAGGGRKHPRYRCRSMGFPKHCGNGTVAMAEWDAFCEEQVLDLLGDAERLEKVWVAG |
| | | SDSAVELAEVNAELVDLTSLIGSPAYRAGSPQREALDARIAALAARQEELEGLEARPSG |
| | | WEWRETGQRFGDWWREQDTAAKNTWLRSMNVRLTFDVRGGLTRTIDFGDLQEYEQHLRL |
| | | GSVVERLHTGMSGSGKDGKKDKKEEDKK* |
| 672 | PL2094_ | ATGCCCAAGAAGAAGAGAAAGGTGTACCCCTACGACGTGCCCGACTACGCCAAGGACGG |
| | DNA | CAAGAAGGACAAGAAGGAGGAGGACAAGAAGAAGGACGGCAAGAAGGACAAGAAGGAGG |
| | | AGGACAAGAAGGGCAGCAGAGCCCTGGTGGTGATCAGACTGAGCAGAGTGACCGACGCC |
| | | ACCACCAGCCCCGAGAGACAGCTGGAGAGCTGCCAGCAGCTGTGCGCCCAGAGAGGCTG |
| | | GGACGTGGTGGGCGTGGCCGAGGACCTGGACGTGAGCGGCGCCGTGGACCCCTTCGACA |
| | | GAAAGAGAAGACCCAACCTGGCCAGATGGCTGGCCTTCGAGGAGCAGCCCTTCGACGTG |
| | | ATCGTGGCCTACAGAGTGGACAGACTGACCAGAAGCATCAGACACCTGCAGCAGCTGGT |
| | | GCACTGGGCCGAGGACCACAAGAAGCTGGTGGTGAGCGCCACCGAGGCCCACTTCGACA |
| | | CCACCACCCCCTTCGCCGCCGTGGTGATCGCCCTGATGGGCACCGTGGCCCAGATGGAG |
| | | CTGGAGGCCATCAAGGAGAGAAACAGAAGCGCCGCCCACTTCAACATCAGAGCCGGCAA |
| | | GTACAGAGGCAGCCTGCCCCCCTGGGGCTACCTGCCCACCAGAGTGGACGGCGAGTGGA |
| | | GACTGGTGCCCGACCCCGTGCAGAGAGAGAGAATCCTGGAGGTGTACCACAGAGTGGTG |
| | | GACAACCACGAGCCCCTGCACCTGGTGGCCCACGACCTGAACAGAAGAGGCGTGCTGAG |
| | | CCCCAAGGACTACTTCGCCCAGCTGCAGGGCAGAGAGCCCCAGGGCAGAGAGTGGAGCG |
| | | CCACCGCCCTGAAGAGAAGCATGATCAGCGAGGCCATGCTGGGCTACGCCACCCTGAAC |
| | | GGCAAGACCGTGAGAGACGACGACGGCGCCCCCCTGGTGAGAGCCGAGCCCATCCTGAC |
| | | CAGAGAGCAGCTGGAGGCCCTGAGAGCCGAGCTGGTGAAGACCAGCAGAGCCAAGCCCG |
| | | CCGTGAGCACCCCCAGCCTGCTGCTGAGAGTGCTGTTCTGCGCCGTGTGCGGCGAGCCC |
| | | GCCTACAAGTTCGCCGGCGGCGGCAGAAAGCACCCCAGATACAGATGCAGAAGCATGGG |
| | | CTTCCCCAAGCACTGCGGCAACGGCACCGTGGCCATGGCCGAGTGGGACGCCTTCTGCG |
| | | AGGAGCAGGTGCTGGACCTGCTGGGCGACGCCGAGAGACTGGAGAAGGTGTGGGTGGCC |
| | | GGCAGCGACAGCGCCGTGGAGCTGGCCGAGGTGAACGCCGAGCTGGTGGACCTGACCAG |
| | | CCTGATCGGCAGCCCCGCCTACAGAGCCGGCAGCCCCCAGAGAGAGGCCCTGGACGCCA |
| | | GAATCGCCGCCCTGGCCGCCAGACAGGAGGAGCTGGAGGGCCTGGAGGCCAGACCCAGC |
| | | GGCTGGGAGTGGAGAGAGACCGGCCAGAGATTCGGCGACTGGTGGAGAGAGCAGGACAC |
| | | CGCCGCCAAGAACACCTGGCTGAGAAGCATGAACGTGAGACTGACCTTCGACGTGAGAG |
| | | GCGGCCTGACCAGAACCATCGACTTCGGCGACCTGCAGGAGTACGAGCAGCACCTGAGA |
| | | CTGGGCAGCGTGGTGGAGAGACTGCACACCGGCATGAGCGGCAGCGGCAAGGACGGCAA |
| | | GAAGGACAAGAAGGAGGAGGACAAGAAGTGA |
| 673 | PL2095 | MPKKKRKVYPYDVPDYAKDGKKDKKEEDKKKDGKKDKKEEDKKGSRALVVIRLSRVTDA |
| | _AA | TTSPERQLESCQQLCAQRGWDVVGVAEDLDVSGAVDPFDRKRRPNLARWLAFEEQPFDV |
| | | IVAYRVDRLTRSIRHLQQLVHWAEDHKKLVVSATEAHFDTTTPFAAVVIALMGTVAQME |
| | | LEAIKERNRSAAHFNIRAGKYRGSLPPWGYLPTRVDGEWRLVPDPVQRERILEVYHRWD |
| | | NHEPLHLVAHDLNRRGVLSPKDYFAQLQGREPQGREWSATALKRSMISEAMLGYATLNG |
| | | KTVRDDDGAPLVRAEPILTREQLEALRAELVKTSRAKPAVSTPSLLLRVLFCAVCGEPA |
| | | YKFAGGGRKHPRYRCRSMGFPKHCGNGTVAMAEWDAFCEEQVLDLLGDAERLEKVWVAG |
| | | SDSAVELAEVNAELVDLTSLIGSPAYRAGSPQREALDARIAALAARQEELEGLEARPSG |
| | | WEWRETGQRFGDWWREQDTAAKNTWLRSMNVRLTFDVRGGLTRTIDFGDLQEYEQHLRL |
| | | GSVVERLHTGMSGSGKDGKKDKKEEDKKKDGKKDKKEEDKK* |
| 674 | PL2095_ | ATGCCCAAGAAGAAGAGAAAGGTGTACCCCTACGACGTGCCCGACTACGCCAAGGACGG |
| | DNA | CAAGAAGGACAAGAAGGAGGAGGACAAGAAGAAGGACGGCAAGAAGGACAAGAAGGAGG |
| | | AGGACAAGAAGGGCAGCAGAGCCCTGGTGGTGATCAGACTGAGCAGAGTGACCGACGCC |
| | | ACCACCAGCCCCGAGAGACAGCTGGAGAGCTGCCAGCAGCTGTGCGCCCAGAGAGGCTG |
| | | GGACGTGGTGGGCGTGGCCGAGGACCTGGACGTGAGCGGCGCCGTGGACCCCTTCGACA |
| | | GAAAGAGAAGACCCAACCTGGCCAGATGGCTGGCCTTCGAGGAGCAGCCCTTCGACGTG |
| | | ATCGTGGCCTACAGAGTGGACAGACTGACCAGAAGCATCAGACACCTGCAGCAGCTGGT |
| | | GCACTGGGCCGAGGACCACAAGAAGCTGGTGGTGAGCGCCACCGAGGCCCACTTCGACA |
| | | CCACCACCCCCTTCGCCGCCGTGGTGATCGCCCTGATGGGCACCGTGGCCCAGATGGAG |
| | | CTGGAGGCCATCAAGGAGAGAAACAGAAGCGCCGCCCACTTCAACATCAGAGCCGGCAA |
| | | GTACAGAGGCAGCCTGCCCCCCTGGGGCTACCTGCCCACCAGAGTGGACGGCGAGTGGA |
| | | GACTGGTGCCCGACCCCGTGCAGAGAGAGAGAATCCTGGAGGTGTACCACAGAGTGGTG |
| | | GACAACCACGAGCCCCTGCACCTGGTGGCCCACGACCTGAACAGAAGAGGCGTGCTGAG |
| | | CCCCAAGGACTACTTCGCCCAGCTGCAGGGCAGAGAGCCCCAGGGCAGAGAGTGGAGCG |
| | | CCACCGCCCTGAAGAGAAGCATGATCAGCGAGGCCATGCTGGGCTACGCCACCCTGAAC |
| | | GGCAAGACCGTGAGAGACGACGACGGCGCCCCCCTGGTGAGAGCCGAGCCCATCCTGAC |
| | | CAGAGAGCAGCTGGAGGCCCTGAGAGCCGAGCTGGTGAAGACCAGCAGAGCCAAGCCCG |
| | | CCGTGAGCACCCCCAGCCTGCTGCTGAGAGTGCTGTTCTGCGCCGTGTGCGGCGAGCCC |
| | | GCCTACAAGTTCGCCGGCGGCGGCAGAAAGCACCCCAGATACAGATGCAGAAGCATGGG |
| | | CTTCCCCAAGCACTGCGGCAACGGCACCGTGGCCATGGCCGAGTGGGACGCCTTCTGCG |
| | | AGGAGCAGGTGCTGGACCTGCTGGGCGACGCCGAGAGACTGGAGAAGGTGTGGGTGGCC |
| | | GGCAGCGACAGCGCCGTGGAGCTGGCCGAGGTGAACGCCGAGCTGGTGGACCTGACCAG |
| | | CCTGATCGGCAGCCCCGCCTACAGAGCCGGCAGCCCCCAGAGAGAGGCCCTGGACGCCA |
| | | GAATCGCCGCCCTGGCCGCCAGACAGGAGGAGCTGGAGGGCCTGGAGGCCAGACCCAGC |
| | | GGCTGGGAGTGGAGAGAGACCGGCCAGAGATTCGGCGACTGGTGGAGAGAGCAGGACAC |
| | | CGCCGCCAAGAACACCTGGCTGAGAAGCATGAACGTGAGACTGACCTTCGACGTGAGAG |
| | | GCGGCCTGACCAGAACCATCGACTTCGGCGACCTGCAGGAGTACGAGCAGCACCTGAGA |
| | | CTGGGCAGCGTGGTGGAGAGACTGCACACCGGCATGAGCGGCAGCGGCAAGGACGGCAA |
| | | GAAGGACAAGAAGGAGGAGGACAAGAAGAAGGACGGCAAGAAGGACAAGAAGGAGGAGG |
| | | ACAAGAAGTGA |
| 675 | PL2096 | MPKKKRKVKDGKKDKKEEDKKKDGKKDKKEEDKKGSRALVVIRLSRVTDATTSPERQLE |
| | _AA | SCQQLCAQRGWDWVGVAEDLDVSGAVDPFDRKRRPNLARWLAFEEQPFDVIVAYRVDRL |
| | | TRSIRHLQQLVHWAEDHKKLVVSATEAHFDTTTPFAAVVIALMGTVAQMELEAIKERNR |
| | | SAAHFNIRAGKYRGSLPPWGYLPTRVDGEWRLVPDPVQRERILEVYHRVVDNHEPLHLV |
| | | AHDLNRRGVLSPKDYFAQLQGREPQGREWSATALKRSMISEAMLGYATLNGKTVRDDDG |
| | | APLVRAEPILTREQLEALRAELVKTSRAKPAVSTPSLLLRVLFCAVCGEPAYKFAGGGR |
| | | KHPRYRCRSMGFPKHCGNGTVAMAEWDAFCEEQVLDLLGDAERLEKVWVAGSDSAVELA |
| | | EVNAELVDLTSLIGSPAYRAGSPQREALDARIAALAARQEELEGLEARPSGWEWRETGQ |
| | | RFGDWWREQDTAAKNTWLRSMNVRLTFDVRGGLTRTIDFGDLQEYEQHLRLGSVVERLH |
| | | TGMSGSGKDGKKDKKEEDKKKDGKKDKKEEDKK* |
| 676 | PL2096_ | ATGCCCAAGAAGAAGAGAAAGGTGAAGGACGGCAAGAAGGACAAGAAGGAGGAGGACAA |
| | DNA | GAAGAAGGACGGCAAGAAGGACAAGAAGGAGGAGGACAAGAAGGGCAGCAGAGCCCTGG |
| | | TGGTGATCAGACTGAGCAGAGTGACCGACGCCACCACCAGCCCCGAGAGACAGCTGGAG |
| | | AGCTGCCAGCAGCTGTGCGCCCAGAGAGGCTGGGACGTGGTGGGCGTGGCCGAGGACCT |
| | | GGACGTGAGCGGCGCCGTGGACCCCTTCGACAGAAAGAGAAGACCCAACCTGGCCAGAT |
| | | GGCTGGCCTTCGAGGAGCAGCCCTTCGACGTGATCGTGGCCTACAGAGTGGACAGACTG |
| | | ACCAGAAGCATCAGACACCTGCAGCAGCTGGTGCACTGGGCCGAGGACCACAAGAAGCT |
| | | GGTGGTGAGCGCCACCGAGGCCCACTTCGACACCACCACCCCCTTCGCCGCCGTGGTGA |
| | | TCGCCCTGATGGGCACCGTGGCCCAGATGGAGCTGGAGGCCATCAAGGAGAGAAACAGA |
| | | AGCGCCGCCCACTTCAACATCAGAGCCGGCAAGTACAGAGGCAGCCTGCCCCCCTGGGG |
| | | CTACCTGCCCACCAGAGTGGACGGCGAGTGGAGACTGGTGCCCGACCCCGTGCAGAGAG |
| | | AGAGAATCCTGGAGGTGTACCACAGAGTGGTGGACAACCACGAGCCCCTGCACCTGGTG |
| | | GCCCACGACCTGAACAGAAGAGGCGTGCTGAGCCCCAAGGACTACTTCGCCCAGCTGCA |
| | | GGGCAGAGAGCCCCAGGGCAGAGAGTGGAGCGCCACCGCCCTGAAGAGAAGCATGATCA |
| | | GCGAGGCCATGCTGGGCTACGCCACCCTGAACGGCAAGACCGTGAGAGACGACGACGGC |
| | | GCCCCCCTGGTGAGAGCCGAGCCCATCCTGACCAGAGAGCAGCTGGAGGCCCTGAGAGC |
| | | CGAGCTGGTGAAGACCAGCAGAGCCAAGCCCGCCGTGAGCACCCCCAGCCTGCTGCTGA |
| | | GAGTGCTGTTCTGCGCCGTGTGCGGCGAGCCCGCCTACAAGTTCGCCGGCGGCGGCAGA |
| | | AAGCACCCCAGATACAGATGCAGAAGCATGGGCTTCCCCAAGCACTGCGGCAACGGCAC |
| | | CGTGGCCATGGCCGAGTGGGACGCCTTCTGCGAGGAGCAGGTGCTGGACCTGCTGGGCG |
| | | ACGCCGAGAGACTGGAGAAGGTGTGGGTGGCCGGCAGCGACAGCGCCGTGGAGCTGGCC |
| | | GAGGTGAACGCCGAGCTGGTGGACCTGACCAGCCTGATCGGCAGCCCCGCCTACAGAGC |
| | | CGGCAGCCCCCAGAGAGAGGCCCTGGACGCCAGAATCGCCGCCCTGGCCGCCAGACAGG |
| | | AGGAGCTGGAGGGCCTGGAGGCCAGACCCAGCGGCTGGGAGTGGAGAGAGACCGGCCAG |
| | | AGATTCGGCGACTGGTGGAGAGAGCAGGACACCGCCGCCAAGAACACCTGGCTGAGAAG |
| | | CATGAACGTGAGACTGACCTTCGACGTGAGAGGCGGCCTGACCAGAACCATCGACTTCG |
| | | GCGACCTGCAGGAGTACGAGCAGCACCTGAGACTGGGCAGCGTGGTGGAGAGACTGCAC |
| | | ACCGGCATGAGCGGCAGCGGCAAGGACGGCAAGAAGGACAAGAAGGAGGAGGACAAGAA |
| | | GAAGGACGGCAAGAAGGACAAGAAGGAGGAGGACAAGAAGTGA |

### 9. EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

It is an object of the invention not to encompass within the invention any previously known product, process of making the product, or method of using the product such that Applicant reserves the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. 112(a)) or the EPO (Article 83 of the EPC), such that Applicant reserves the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise. It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

While the invention has been particularly shown and described with reference to a preferred embodiment and various alternate embodiments, it is understood by persons skilled in the relevant art that various changes in form and details can be made therein without departing from the spirit and scope of the invention.

1. An engineered large serine integrase (eLSR) comprising one or more substitutions that substantially maintain or enhance integration activity at a pair of cognate integration recognition sites, and substantially decrease off-target integration activity at a pair of off-target integration recognition sites, when compared to a corresponding large serine integrase without said one or more substitutions (cLSR); optionally, said one or more substitutions are in a zinc ribbon domain (ZD) of the cLSR.

2. The eLSR of claim 1, wherein said cLSR comprises an amino acid sequence that is at least 80% identical to any one of: (a) SEQ ID NOs: 378-393; (b) SEQ ID NOs: 85-158 of WO2023/177424; and (c) SEQ ID NOs: 1-16 and 163-1162 and 3166-3175 of WO2023/070031.

3.-38. (canceled)

39. The eLSR of claim 1, wherein the eLSR is linked to a gene editor polypeptide.

40.-44. (canceled)

45. A polynucleotide comprising a nucleic acid sequence encoding the eLSR of claim 1.

46. The polynucleotide of claim 45, wherein the nucleic acid sequence encoding the eLSR and/or the fusion is codon optimized (e.g., codon-optimized for expression in a mammalian cell, such as a human cell).

47.-49. (canceled)

50. A vector comprising the polynucleotide of claim 45.

51. A host cell comprising the vector of claim 50.

52. A fusion protein, comprising:
(a) a DNA binding domain, optionally comprising a nickase activity;
(b) a reverse transcriptase; and
(c) an eLSR of claim 1,
wherein at least any two of elements (a), (b), or (c) are linked via at least a first C-terminal linker.

53. The fusion protein of claim 52, wherein the C-terminal linker comprises a sequence in Table 3.

54. A polynucleotide comprising a nucleic acid sequence encoding the fusion protein of claim 52.

55. A vector comprising the polynucleotide of claim 54.

56. A host cell comprising the vector of claim 55.

57. A system for site-specifically integrating a donor polynucleotide template into a mammalian cell genome at a target DNA sequence, comprising:
(1) an attachment site containing gRNA (atgRNA) comprising at least a portion of an at least first integration recognition site;
(2) a gene editor polypeptide comprising a DNA binding nickase domain linked to a reverse transcriptase domain capable of incorporating the integration recognition site into the target DNA sequence;
(3) an eLSR of claim 1; and
(4) a donor polynucleotide template linked to a sequence that is an integration cognate of the integration recognition site present in the atgRNA,
whereby the gene editor polypeptide site-specifically integrates the integration recognition site into the target DNA sequence, and,
whereby the eLSR integrates the donor polynucleotide template into the target DNA sequence at the integration recognition site.

58. The system of claim 57, wherein the first atgRNA comprises:
(i) a domain that is capable of guiding the gene editor polypeptide to the target DNA sequence; and
(ii) a reverse transcriptase (RT) template that comprises at least a portion of an at least first integration recognition site,
whereby the at least portion of the at least first integration recognition site is integrated into the genome of the cell at the target sequence.

59.-61. (canceled)

62. A method for site-specifically integrating a donor polynucleotide template into a mammalian cell genome at a target DNA sequence, comprising:
(1) incorporating an integration recognition site into the genome by delivering into the cell:
i) an attachment site-containing guide RNA (atgRNA) comprising at least a portion of an at least first integration recognition site; and
ii) a gene editor polypeptide or polynucleotide encoding the gene editor polypeptide, wherein the gene editor polypeptide comprises a DNA binding nickase domain linked to a reverse transcriptase domain, and is capable of incorporating the integration recognition site into the target DNA sequence; and
iii) optionally, a nicking gRNA; and
(2) integrating the donor polynucleotide template into the genome by delivering into the cell:
a) an eLSR of any one of claim 1; and
b) a donor polynucleotide template, wherein the donor polynucleotide template is linked to a sequence that is an integration cognate of the integration recognition site present in the atgRNA, and wherein the donor polynucleotide template is integrated into the genome at the incorporated genomic integration recognition site by the eLSR.

63. The method of claim 62, wherein the atgRNA, the gene editor polypeptide or polynucleotide encoding the gene editor polypeptide, the optional nicking gRNA, the eLSR, and the donor polynucleotide template are introduced into the cell concurrently.

64.-70. (canceled)

71. A cell comprising the fusion protein of claim 52.

72. The cell of claim 71, wherein the cell is a pleiopluripotent cell (such as an induced pluripotent stem cell) or a hematopoietic cell differentiated from a pleiopluripotent cell.

73.-75. (canceled)
